(19) United States
(12) Patent Application Publication
(10) Pub. No.: US 2026/0365653 A1
(43) Pub. Date: Nov. 19, 2026

(54) IRREVERSIBLE MUTEGFR INHIBITORS

(71) Applicants: Bayer Aktiengesellschaft, Leverkusen (DE); The Broad Institute, Inc., Cambridge, MA (US); Dana-Farber Cancer Institute, Inc., Boston, MA (US)

(72) Inventors: Stephan Siegel, Berlin (DE); Simon Anthony Herbert, Berlin (DE); Carsten Schmeck, Mülheim (DE); Franziska Siegel, Berlin (DE); Jeremie Xavier G. Mortier, Berlin (DE); Stefanie Zimmermann, Düsseldorf (DE); Hans Briem, Bremen (DE); Benjamin Bader, Berlin (DE); Ulf Bömer, Glienicke (DE); Jens Schröder, Weimar (DE); Douglas Orsi, Cambridge, MA (US); Heidi Greulich, Cambridge, MA (US); Alexandra Gould, Cambridge, MA (US); Matthew Meyerson, Boston, MA (US); Alexander Konstantin Christian Ulrich, Berlin (DE); Roman Hillig, Berlin (DE); Daniel Korr, Berlin (DE); Christian Lechner, Berlin (DE); Laura Evans, Somerville, MA (US)

(21) Appl. No.: 18/932,983

(22) PCT Filed: May 3, 2023
(86) PCT No.: PCT/EP2023/061672

§ 371 Date: Oct. 31, 2024

Related U.S. Application Data
(60) Provisional application No. 63/338,419, filed on May 4, 2022.

(51) Int. Cl. C07D 487/04; A61K 31/4162; A61K 45/06; C07D 519/00; C07K 16/28

(52) U.S. Cl. CPC C07D 487/04; A61K 31/4162; A61K 45/06; C07D 519/00; C07K 16/2863

(57) ABSTRACT

Compounds of formula (I) processes for their production and their use as pharmaceuticals.

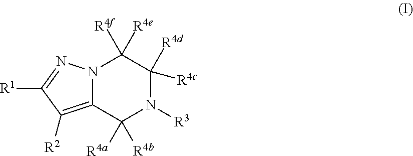

### STATEMENT OF RIGHTS TO INVENTIONS MADE UNDER FEDERALLY SPONSORED RESEARCH

This invention was made with government support under Grant Nos. R01CA116020 and 5P01CA154303 awarded by the National Institutes of Health. The government has certain rights in the invention.

### FIELD OF APPLICATION OF THE INVENTION

The present invention covers 6,7-dihydropyrazolo[1,5-a]pyrazin derivatives of formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular cancer, as a sole agent or in combination with other active ingredients.

### BACKGROUND OF THE INVENTION

The present invention covers 6,7-dihydropyrazolo[1,5-a]pyrazin derivatives of formula (I) which inhibit EGFR.

The Epidermal Growth Factor Receptor (EGFR or EGF-receptor) receptor tyrosine kinase family consists of 4 members: EGFR (Erbb1, Her1), ERBB2 (Her2), ERBB3 (Her3), and ERBB4 (Her4). EGFR mediates activation of MAPK and PI3K signaling pathways and thereby regulates cell proliferation, differentiation, migration and survival (Pao et al., 2010). EGFR gene amplification, overexpression, and mutations are frequently observed in various cancer indications and are associated with a poor prognosis (Gridelli et al., 2015).

In lung adenocarcinoma, mutations of EGFR are prevalent in approximately 15% of Western patients and up to 50% of East Asian patients (Paez et al., 2004). These mutations typically occur in one of four exons, exons 18-21, in the kinase domain of EGFR (Paez et al., 2004). The most common activating mutations in EGFR are a point mutation in exon 21, substituting an arginine for a leucine (L858R), and a small in-frame deletion in exon 19 that removes four amino acids (del 19/del746-750) (Pao et al., 2010). The FDA-approved inhibitors gefitinib, erlotinib, and afatinib, targeting mutations in exons 18, 19, and 21 of EGFR, are effective in patients, but the response is often not durable (Mok et al., 2009; Sequist et al., 2013). Resistance frequently occurs in these patients in response to acquisition of a second mutation, T790M (Pao et al., 2005). Second generation inhibitors, e.g. afatinib, irreversibly target this mutation, but are still potent inhibitors of wild-type EGFR, leading to dose-limiting toxicity and lack of efficacy in patients.

Several irreversible EGFR inhibitors are published in CN 110857292, IN 201821027709, CN 110698461, WO 2020001351, WO 2020001350, WO 2019233459, CN 110407852, CN 110357863, WO 2019070167, WO20061470. WO2019/081486 describes 4H-Pyrrolo[3,2-c]pyridine-4-one derivatives.

A third-generation irreversible inhibitor, osimertinib, that maximizes activity towards T790M while minimizing activity towards wild-type EGFR, is effective in T790M mutant patients and is currently the standard treatment for T790M positive patients (Mok et al., 2017). Osimertinib is also approved as a front-line therapy for patients with mutations of EGFR exons 19 or 21 (Soria et al., 2018).

By contrast, and with the exception of A763_Y764insFQEA, small in-frame insertions of EGFR exon20 are resistant to the classical EGFR inhibitors at doses achievable in lung cancer patients and comprise an unmet medical need (Yasuda et. al., 2013).

Patients with EGFR exon20 insertions, such as V769_D770insASV, D770_N771insSVD, D770_N771insNPG, N771_P772insH, H773_V774insH, H773_V774insNPH, V774_C775insHV show particular low response rates to EGFR-targeted therapies, resulting in significantly reduced progression-free survival as well as overall survival (Chen et al., 2016). This has been shown for the first-generation inhibitors erlotinib and gefitinib as well as for the second-generation inhibitor afatinib (Chen et al., 2016; Yang et al., 2015). The same resistance profile has been observed for exon20 insertion mutations in ERBB2 (e.g. ERBB2 A775_G776insYVMA with the highest prevalence), another member of the EGF-receptor family (Arcila et al., 2012) and some of the uncommon EGFR mutations like L681Q (Chiu et al., 2015).

The standard of care for EGFR exon20 insertion patients is currently chemotherapy. However, amivantamab and mobocertinib received accelerated approval for 2nd line treatment post chemotherapy recently, and several other inhibitors are currently in clinical trials for the treatment of EGFR exon20 insertion mutation positive lung cancer patients (Friedlaender et al., 2022).

About 40% of patients with advanced EGFR mutant NSCLC develop brain metastases over the course of their disease (Rangachari et al., 2015). The 1^{st} and 2nd generation EGFR inhibitors show only limited brain permeability. The 3^{rd} generation EGFR inhibitor Osimertinib shows clearly improved CNS activity and is currently the preferred treatment option for patients with classical activating EGFR mutations and brain metastasis (Reungwetwattana et al., 2018).

However, Osimertinib has only limited activity on EGFR exon20 insertion mutations. Furthermore, the recently approved bispecific antibody amivantamab and also mobocertinib show only limited blood-brain-barrier permeability. So there still remains a high unmet medical need especially for lung cancer patients carrying EGFR exon20 insertion mutations and brain metastasis.

In summary, mutant EGFR is a promising drug target for cancer therapy. In particular, patients with primary resistance to approved anti-EGFR therapies, due to EGFR exon20 insertions and with brain metastases, have only few treatment options to date and there is a great need for novel alternative and/or improved therapeutics to provide these patients with an efficacious, well-tolerable therapy. Therefore, potent inhibitors of mutant EGFR, particularly of mutant EGFR with exon20 insertion mutations that show improved permeability of the blood-brain-barrier and CNS activity, represent valuable compounds that should complement therapeutic options either as single agents or in combination with other drugs.

### SUMMARY OF THE INVENTION

The invention provides compounds that inhibit a mutant EGFR; specifically, an EGFR comprising one or more exon 20 insertion mutations, an L858R mutation, or a small in-frame deletion of exon 19, in the presence or absence of a T790M mutation and show brain permeability.

It has now been found that the compounds of the present invention have surprising and advantageous properties.

In particular, said compounds of the present invention have surprisingly been found to effectively inhibit mutant EGFR with exon 20 insertion mutations, particularly those harboring a D770_N771ins SVD exon 20 insertion. Furthermore it has been found that these compounds additionally show high cellular potency in EGFR V769_D770insASV, D770_N771insSVD, D770_N771insNPG, N771_P772insH, or H773_V774insNPH exon 20 insertion harboring BA/F3 cell lines.

Surprisingly, the here described compounds retain high cellular activity in BA/F3 cell lines harboring D770_N771insSVD and the T790M mutation.

In addition, the here described compounds potently inhibit proliferation of BA/F3 cell lines carrying EGFR activating mutations with or without T790M acquired resistance mutations (EGFR E746_A750del, L858R, E746_A750del T790M, L858R T790M).

Based on the described properties the here described compounds can therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses mediated by mutant EGFR with exon 20 insertion mutations, a L858R mutation, or a small in-frame deletion of exon 19 (e.g. EGFR E746_A750del) in the presence or absence of a T790M mutation and/or reduce (or block) proliferation in cells harboring EGFR with exon 20 insertion mutations, a L858R mutation, or a small in-frame deletion of exon 19 (e.g. EGFR E746_A750del) in the presence or absence of a T790M mutation, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

### DESCRIPTION OF THE INVENTION

In accordance with a first aspect, the invention relates to compounds of formula (I),

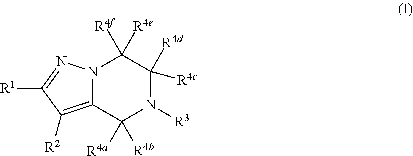

in which:
R¹ represents a group selected from the group:

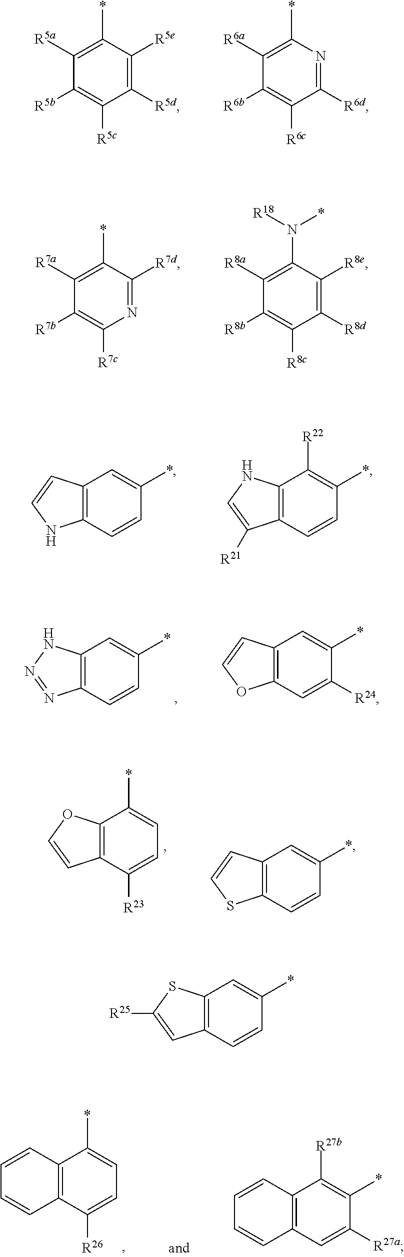

R² represents a group selected from the group:

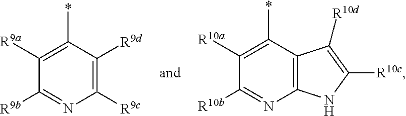

R³ represents —(CO)—R¹¹, —(CO)—C═C—R¹², —(SO₂)—CH═CH₂, or oxirane-2-carbonyl;
R^{4a} represents a hydrogen atom, fluoro, or methyl;
R^{4b} represents a hydrogen atom, fluoro, or methyl;
R^{4c} represents a hydrogen atom, fluoro, or methyl;
R^{4d} represents a hydrogen atom, fluoro, or methyl;
R^{4e} represents a hydrogen atom, fluoro, or methyl;
R^{4f} represents a hydrogen atom, fluoro, methyl, C₁-C₂-hydroxyalkyl, —(CO)—NR¹⁹R²⁰, or —CH₂—(CO)—NR¹⁹R²⁰;
R^{5a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, hydroxy, cyano, fluoro, chloro, or bromo;
R^{5b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, trifluoromethylsulfanyl, cyano, fluoro, chloro, or bromo;
R^{5d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5e} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{6c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₆-alkyl, (C₁-C₃-alkyl)₂amino-C₁-C₃-alkyl, cyano, fluoro, chloro, or bromo;
R^{6d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₆-alkyl, (C₁-C₆-alkyl)₂amino-C₁-C₃-alkyl, or cyano;
R^{7b} represents a hydrogen atom, amino, C₁-C₆-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{7c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{8a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₆-alkyl, (C₁-C₃-alkyl)₂amino-C₁-C₃-alkyl, cyano, fluoro, chloro, or bromo;
R^{8d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8e} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{9a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, C₃-C₃-cycloalkyl, cyano, fluoro, chloro, or bromo;
R^{9b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{9c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, C₁-C₃-alkyl-amino, amino-C₁-C₃-alkyl, C₁-C₃-alkylamino-C₁-C₃-alkyl, (C₁-C₃-alkyl)₂amino-C₁-C₃-alkyl, cyano, anilino, pyridin-2-yl-amino, or C₃-C₆-cycloalkylformamido;
R^{8d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{10a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{10b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{10c} represents a hydrogen atom or methyl;
R^{10d} represents a hydrogen atom, fluoro, or methyl;
R¹¹ represents —CH₂—C≡CH, —C(R¹⁴)—R¹⁵, or

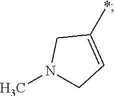

R¹² represents a hydrogen atom, methyl, —CH₂—NR¹⁹R²⁰, or a group selected from the group:

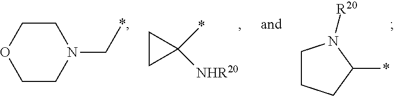

R¹⁴ represents ═CHR¹⁶;
R¹⁵ represents a hydrogen atom, C₁-C₃-alkyl, fluoro, dimethylamino-methyl, or morpholino-methyl;
R¹⁶ represents a hydrogen atom, methyl, chloro, C₁-C₃-haloalkyl, C₁-C₃-hydroxyalkyl, C₁-C₃-alkoxy-C₁-C₃-alkyl, —CH₂—NR^{17a}—CR^{17b}R^{17c}R^{17d}, or a group selected from the group:

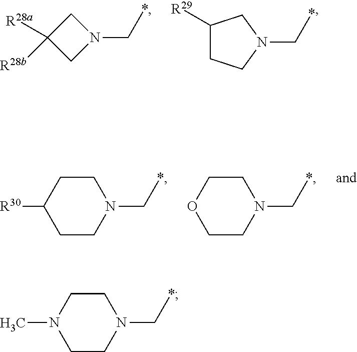

R^{17a} represents a hydrogen atom, methyl, or ethyl;
R^{17b} represents a hydrogen atom or methyl;
R^{17c} represents a hydrogen atom or methyl;
R^{17d} represents a hydrogen atom, methyl, hydroxymethyl, or methoxymethyl;
R¹⁸ represents a hydrogen atom or methyl;
R¹⁹ represents a hydrogen atom or methyl;
R²⁰ represents a hydrogen atom, C₁-C₄-alkyl, or tert-butoxycarbonyl;
R²¹ represents a hydrogen atom or methyl;
R²² represents a hydrogen atom or fluoro;
R²³ represents a hydrogen atom or fluoro;
R²⁴ represents a hydrogen atom or fluoro;
R²⁵ represents a hydrogen atom, fluoro, or chloro;
R²⁶ represents a hydrogen atom or fluoro;
R^{27a} represents a hydrogen atom or fluoro;
R^{27b} represents a hydrogen atom or fluoro;
R^{28a} represents a hydrogen atom or fluoro;
R^{28b} represents a hydrogen atom or fluoro;
R²⁹ represents a hydrogen atom or methoxy;
R³⁰ represents a hydrogen atom or methoxy;
wherein * indicates the point of attachment of said group with the rest of the molecule;
or an N-oxide, a salt, a tautomer, a rotamer, or a stereoisomer of said compound, or a salt of said N-oxide, tautomer, rotamer, or stereoisomer.

In a second aspect, the invention relates to compounds of formula (I) as described supra, wherein:
R¹ represents a group selected from the group:

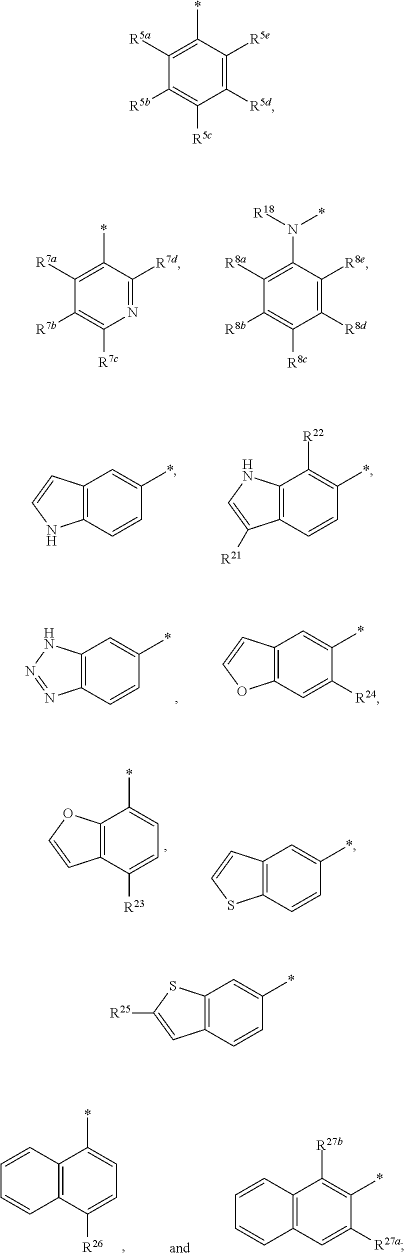

R² represents a group selected from the group:

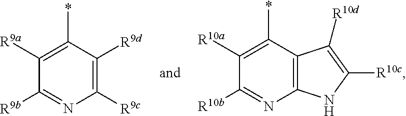

R³ represents —(CO)—R¹¹, —(CO)—C═C—R¹², —(SO₂)—CH═CH₂, or oxirane-2-carbonyl;
R^{4a} represents a hydrogen atom, or methyl;
R^{4b} represents a hydrogen atom;
R^{4c} represents a hydrogen atom, or methyl;
R^{4d} represents a hydrogen atom;
R^{4e} represents a hydrogen atom, or methyl;
R^{4f} represents a hydrogen atom, methyl, C₁-C₂-hydroxyalkyl, —(CO)—NR¹⁹R²⁰, or —CH₂—(CO)—NR¹⁹R²⁰.
R^{5a} represents a hydrogen atom, methyl, hydroxymethyl, methoxy, hydroxy, fluoro, or chloro;
R^{5b} represents a hydrogen atom, C₁-C₃-alkyl, trifluoromethyl, hydroxymethyl, methoxy, cyano, fluoro, or chloro;
R⁵⁰ represents a hydrogen atom, C₁-C₂-alkyl, ethinyl, C₁-haloalkyl, C₁-C₂-alkoxy, C₁-haloalkoxy, trifluoromethylsulfanyl, cyano, fluoro, or chloro;
R^{5d} represents a hydrogen atom, or chloro;
R^{5e} represents a hydrogen atom;
R^{7a} represents a hydrogen atom;
R^{7b} represents a hydrogen atom;
R^{7c} represents trifluoromethyl;
R^{7d} represents a hydrogen atom;
R^{8a} represents a hydrogen atom, or fluoro;
R^{8b} represents a hydrogen atom, or fluoro;
R^{8c} represents a hydrogen atom, methoxy, trifluoromethyl, fluoro, or chloro;
R^{8d} represents a hydrogen atom;
R^{8e} represents a hydrogen atom;
R^{9a} represents a hydrogen atom;
R^{9b} represents a hydrogen atom;
R^{9c} represents a hydrogen atom, amino, methoxy, methyl-amino, anilino, or cyclopropylformamido;
R^{9d} represents a hydrogen atom, methyl, methoxy, or cyclopropyl;
R^{10a} represents a hydrogen atom, or fluoro;
R^{10b} represents a hydrogen atom;
R^{10c} represents a hydrogen atom or methyl;
R^{10d} represents a hydrogen atom, fluoro, or methyl;
R¹¹ represents —C(R¹⁴)—R¹⁵, or

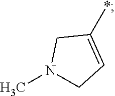

R¹² represents a hydrogen atom, methyl, —CH₂—NR¹⁹R²⁰, or a group selected from the group:

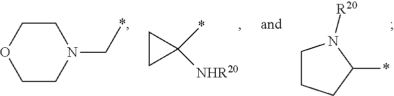

R¹⁴ represents ═CHR¹⁶;
R¹⁵ represents a hydrogen atom, methyl, fluoro, dimethylamino-methyl, or morpholino-methyl;
R¹⁶ represents a hydrogen atom, methyl, chloro, C₁-haloalkyl, hydroxymethyl, methoxymethyl, —CH₂—NR^{17a}—CR^{17b}R¹⁷CR^{17d}, or a group selected from the group:

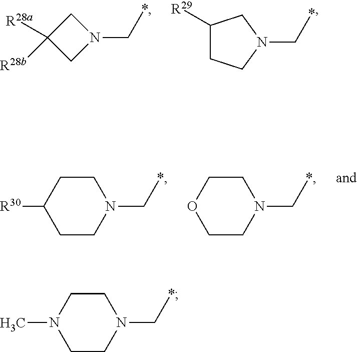

R^{17a} represents a hydrogen atom, methyl, or ethyl;
R^{17b} represents a hydrogen atom or methyl;
R^{17c} represents a hydrogen atom or methyl;
R^{17d} represents a hydrogen atom, methyl, hydroxymethyl, or methoxymethyl;
R¹⁸ represents a hydrogen atom;
R¹⁹ represents a hydrogen atom or methyl;
R²⁰ represents a hydrogen atom, methyl, or tert-butoxycarbonyl;
R²¹ represents a hydrogen atom or methyl;
R²² represents a hydrogen atom or fluoro;
R²³ represents a hydrogen atom or fluoro;
R²⁴ represents a hydrogen atom or fluoro;
R²⁵ represents a hydrogen atom, fluoro, or chloro;
R²⁶ represents a hydrogen atom or fluoro;
R^{27a} represents a hydrogen atom or fluoro;
R^{27b} represents a hydrogen atom or fluoro;
R^{28a} represents a hydrogen atom or fluoro;
R²⁸⁶ represents a hydrogen atom or fluoro;
R²⁹ represents a hydrogen atom or methoxy;
R³⁰ represents a hydrogen atom or methoxy;
wherein * indicates the point of attachment of said group with the rest of the molecule;
or an N-oxide, a salt, a tautomer, a rotamer, or a stereoisomer of said compound, or a salt of said N-oxide, tautomer, rotamer, or stereoisomer.

In a third aspect, the invention relates to compounds of formula (I) as described supra, wherein:
R¹ represents a group selected from the group:

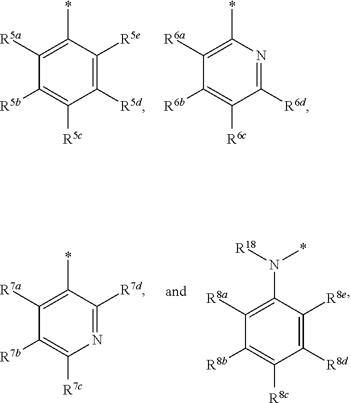

wherein * indicates the point of attachment of said group with the rest of the molecule;
R² represents a group selected from the group:

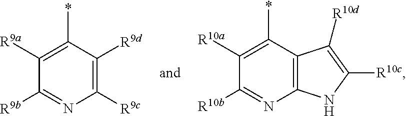

wherein * indicates the point of attachment of said group with the rest of the molecule;
R³ represents —(CO)—R¹¹, —(CO)—C═C—R¹², —(SO₂)—CH═CH₂, or oxirane-2-carbonyl;
R^{4a} represents a hydrogen atom, fluoro, or methyl;
R^{4b} represents a hydrogen atom, fluoro, or methyl;
R^{4c} represents a hydrogen atom, fluoro, or methyl;
R^{4d} represents a hydrogen atom, fluoro, or methyl;
R^{4e} represents a hydrogen atom, fluoro, or methyl;
R^{4f} represents a hydrogen atom, fluoro, methyl, C₁-C₃-hydroxyalkyl, —(CO)—NR¹⁹R²⁰, or —CH₂—(CO)—NR¹⁹R²⁰;
R^{5a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁—C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5e} represents a hydrogen atom, amino, C₁-C₃-alkyl, C₂-C₂-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{6c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{7c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁—C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{8a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{9a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{9b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁—C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{9c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, anilino, pyridin-2-ylamino, or C₃-C₆-cycloalkylformamido;
R^{9d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{10a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{10b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{10c} represents a hydrogen atom or methyl;
R^{10d} represents a hydrogen atom or methyl;
R¹¹ represents —CH₂—C═CH, or —C(R¹⁴)—R¹⁵;
R¹² represents a hydrogen atom or methyl;
R¹⁴ represents ═CH₂, ═CH—CH₃, or ═CH—CH₂N(CH₃)—CHR¹⁶R¹⁷;
R¹⁵ represents a hydrogen atom, C₁-C₃-alkyl, or fluoro;
R¹⁶ represents a hydrogen atom or methyl;
R¹⁷ represents a hydrogen atom or methyl;
R¹⁸ represents a hydrogen atom or methyl;
R¹⁹ represents a hydrogen atom or methyl;
R²⁰ represents a hydrogen atom or methyl;
or an N-oxide, a salt, a tautomer, a rotamer, or a stereoisomer of said compound, or a salt of said N-oxide, tautomer, rotamer, or stereoisomer.

In a fourth aspect, the invention relates to compounds of formula (I) as described supra, wherein:
R¹ represents a group selected from the group:

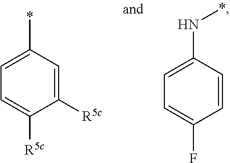

wherein * indicates the point of attachment of said group with the rest of the molecule;
R² represents a group selected from the group:

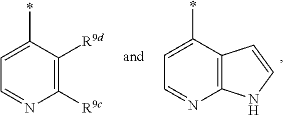

wherein * indicates the point of attachment of said group with the rest of the molecule;
R³ represents —(CO)—R¹¹, —(CO)—C═C—R¹², —(SO₂)—CH═CH₂, or oxirane-2-carbonyl;
R^{5c} represents a hydrogen atom, fluoro, or chloro;
R^{5d} represents a hydrogen atom, methyl, fluoro, or methoxy;
R^{9c} represents a hydrogen atom, amino, methoxy, anilino, or cyclopropylformamido;
R^{9d} represents a hydrogen atom or methoxy;
R¹¹ represents —C(R¹⁴)—R¹⁵;
R¹² represents a hydrogen atom or methyl;
R¹⁴ represents ═CH₂, ═CH—CH₃, or ═CH—CH₂—N(CH₃)—CHR¹⁶R¹⁷;
R¹⁵ represents a hydrogen atom, methyl, or fluoro;
R¹⁶ represents a hydrogen atom or methyl;
R¹⁷ represents a hydrogen atom or methyl;
or an N-oxide, a salt, a tautomer, a rotamer, or a stereoisomer of said compound, or a salt of said N-oxide, tautomer, rotamer, or stereoisomer.

In a fifth aspect, the invention relates to compounds of formula (I) as described supra, which is selected from the group consisting of:
1-[2-(4-fluorophenyl)-3-(pyridin-4-yl)-6,7-dihydropyrazolo[1,5-a]pyrazin-5(4H)-yl]prop-2-en-1-one,
1-[2-(4-chloro-3-methylphenyl)-3-(pyridin-4-yl)-6,7-dihydropyrazolo[1,5-a]pyrazin-5(4H)-yl]prop-2-en-1-one,
1-[2-(4-chlorophenyl)-3-(pyridin-4-yl)-6,7-dihydropyrazolo[1,5-a]pyrazin-5(4H)-yl]prop-2-en-1-one,
1-[2-(3-fluorophenyl)-3-(pyridin-4-yl)-6,7-dihydropyrazolo[1,5-a]pyrazin-5(4H)-yl]prop-2-en-1-one,
1-[2-(3-methoxyphenyl)-3-(pyridin-4-yl)-6,7-dihydropyrazolo[1,5-a]pyrazin-5(4H)-yl]prop-2-en-1-one,
N-{4-[2-(4-fluorophenyl)-5-{(2E)-4-[methyl(propan-2-yl)amino]but-2-enoyl}-4,5,6,7-tetrahydropyrazolo[1,5-a]pyrazin-3-yl]pyridin-2-yl}cyclopropanecarboxamide,
N-{4-[2-(4-fluorophenyl)-5-(prop-2-enoyl)-4,5,6,7-tetrahydropyrazolo[1,5-a]pyrazin-3-yl]pyridin-2-yl}cyclopropanecarboxamide,
N-(4-{5-[(2E)-4-(dimethylamino)but-2-enoyl]-2-(4-fluorophenyl)-4,5,6,7-tetrahydropyrazolo[1,5-a]pyrazin-3-yl}pyridin-2-yl)cyclopropanecarboxamide,
N-{4-[2-(4-fluorophenyl)-5-(oxirane-2-carbonyl)-4,5,6,7-tetrahydropyrazolo[1,5-a]pyrazin-3-yl]pyridin-2-yl}cyclopropanecarboxamide,
1-[3-(2-aminopyridin-4-yl)-2-(4-fluorophenyl)-6,7-dihydropyrazolo[1,5-a]pyrazin-5(4H)-yl]prop-2-en-1-one,
1-[2-(4-fluorophenyl)-3-(2-methoxypyridin-4-yl)-6,7-dihydropyrazolo[1,5-a]pyrazin-5(4H)-yl]prop-2-en-1-one,
1-[2-(4-fluorophenyl)-3-(3-methoxypyridin-4-yl)-6,7-dihydropyrazolo[1,5-a]pyrazin-5(4H)-yl]prop-2-en-1-one,
1-[2-(4-fluorophenyl)-3-(pyridin-4-yl)-6,7-dihydropyrazolo[1,5-a]pyrazin-5(4H)-yl]prop-2-yn-1-one,
5-(ethenesulfonyl)-2-(4-fluorophenyl)-3-(pyridin-4-yl)-4,5,6,7-tetrahydropyrazolo[1,5-a]pyrazine,
(2E)-4-(dimethylamino)-1-[2-(4-fluorophenyl)-3-(pyridin-4-yl)-6,7-dihydropyrazolo[1,5-a]pyrazin-5(4H)-yl]but-2-en-1-one,
1-[2-(4-fluorophenyl)-3-(pyridin-4-yl)-6,7-dihydropyrazolo[1,5-a]pyrazin-5(4H)-yl]but-2-yn-1-one,
2-fluoro-1-[2-(4-fluorophenyl)-3-(pyridin-4-yl)-6,7-dihydropyrazolo[1,5-a]pyrazin-5(4H)-yl]prop-2-en-1-one,
(2E)-1-[2-(4-fluorophenyl)-3-(pyridin-4-yl)-6,7-dihydropyrazolo[1,5-a]pyrazin-5(4H)-yl]-4-[methyl(propan-2-yl)amino]but-2-en-1-one,
1-[2-(4-fluoroanilino)-3-(pyridin-4-yl)-6,7-dihydropyrazolo[1,5-a]pyrazin-5(4H)-yl]prop-2-en-1-one,
1-[3-(2-anilinopyridin-4-yl)-2-(4-fluorophenyl)-6,7-dihydropyrazolo[1,5-a]pyrazin-5(4H)-yl]prop-2-en-1-one,
(2E)-1-[3-(2-anilinopyridin-4-yl)-2-(4-fluorophenyl)-6,7-dihydropyrazolo[1,5-a]pyrazin-5(4H)-yl]-4-(dimethylamino)but-2-en-1-one,
1-[2-(4-fluorophenyl)-3-(1H-pyrrolo[2,3-b]pyridin-4-yl)-6,7-dihydropyrazolo[1,5-a]pyrazin-5(4H)-yl]prop-2-en-1-one,
(2E)-1-[2-(4-fluorophenyl)-3-(pyridin-4-yl)-6,7-dihydropyrazolo[1,5-a]pyrazin-5(4H)-yl]but-2-en-1-one,
1-[2-(4-fluorophenyl)-3-(pyridin-4-yl)-6,7-dihydropyrazolo[1,5-a]pyrazin-5(4H)-yl]-2-methylprop-2-en-1-one,
(2E)-1-[2-(4-chloro-3-fluorophenyl)-3-(pyridin-4-yl)-6,7-dihydropyrazolo[1,5-a]pyrazin-5(4H)-yl]-4-(dimethylamino)but-2-en-1-one
(2E)-1-[2-(4-chloro-2-fluorophenyl)-3-(pyridin-4-yl)-6,7-dihydropyrazolo[1,5-a]pyrazin-5(4H)-yl]-4-(dimethylamino)but-2-en-1-one
(2E)-1-[2-(3-chloro-4-methoxyphenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(2-methoxyphenyl)-3-(pyridin-4-yl)-6,7-dihydropyrazolo[1,5-a]pyrazin-5(4H)-yl]but-2-en-1-one
(2E)-1-[2-(3,4-dichlorophenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(4-methoxyphenyl)-3-(pyridin-4-yl)-6,7-dihydropyrazolo[1,5-a]pyrazin-5(4H)-yl]but-2-en-1-one
(2E)-4-(dimethylamino)-1-[2-(3-fluoro-4-methoxyphenyl)-3-(pyridin-4-yl)-6,7-dihydropyrazolo[1,5-a]pyrazin-5(4H)-yl]but-2-en-1-one
formic acid-(2E)-1-[2-(4-chloro-3-ethylphenyl)-3-(pyridin-4-yl)-6,7-dihydropyrazolo[1,5-a]pyrazin-5(4H)-yl]-4-(dimethylamino)but-2-en-1-one
4-{5-[(2E)-4-(dimethylamino)but-2-enoyl]-3-(pyridin-4-yl)-4,5,6,7-tetrahydropyrazolo[1,5-a]pyrazin-2-yl}benzonitrile
(2E)-4-(dimethylamino)-1-[3-(pyridin-4-yl)-2-[4-(trifluoromethyl)phenyl]-6,7-dihydropyrazolo[1,5-a]pyrazin-5(4H)-yl]but-2-en-1-one
(2E)-1-{2-[4-chloro-2-(hydroxymethyl)phenyl]-3-(pyridin-4-yl)-6,7-dihydropyrazolo[1,5-a]pyrazin-5(4H)-yl}-4-(dimethylamino)but-2-en-1-one
1-[2-(4-chloro-3-fluorophenyl)-3-(pyridin-4-yl)-6,7-dihydropyrazolo[1,5-a]pyrazin-5(4H)-yl]prop-2-en-1-one
1-[2-(4-chloro-2-fluorophenyl)-3-(pyridin-4-yl)-6,7-dihydropyrazolo[1,5-a]pyrazin-5(4H)-yl]prop-2-en-1-one
1-[2-(3-chloro-4-methoxyphenyl)-3-(pyridin-4-yl)-6,7-dihydropyrazolo[1,5-a]pyrazin-5(4H)-yl]prop-2-en-1-one
1-[2-(2-methoxyphenyl)-3-(pyridin-4-yl)-6,7-dihydropyrazolo[1,5-a]pyrazin-5(4H)-yl]prop-2-en-1-one
1-[2-(3,4-dichlorophenyl)-3-(pyridin-4-yl)-6,7-dihydropyrazolo[1,5-a]pyrazin-5(4H)-yl]prop-2-en-1-one
1-[2-(4-methoxyphenyl)-3-(pyridin-4-yl)-6,7-dihydropyrazolo[1,5-a]pyrazin-5(4H)-yl]prop-2-en-1-one
1-[2-(3-fluoro-4-methoxyphenyl)-3-(pyridin-4-yl)-6,7-dihydropyrazolo[1,5-a]pyrazin-5(4H)-yl]prop-2-en-1-one
4-[5-acryloyl-3-(pyridin-4-yl)-4,5,6,7-tetrahydropyrazolo[1,5-a]pyrazin-2-yl]benzonitrile
1-[3-(pyridin-4-yl)-2-[4-(trifluoromethyl)phenyl]-6,7-dihydropyrazolo[1,5-a]pyrazin-5(4H)-yl]prop-2-en-1-one
3-[3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
1-[2-(3-fluoro-4-methoxyphenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(4-methoxyphenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-[2-(4-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(4-chloro-2-fluorophenyl)-3-(3-methylpyridin-4-yl)-6,7-dihydropyrazolo[1,5-a]pyrazin-5(4H)-yl]prop-2-en-1-one
1-[2-(4-chloro-2-fluorophenyl)-3-(5-fluoro-1H-pyrrolo[2,3-b]pyridin-4-yl)-6,7-dihydropyrazolo[1,5-a]pyrazin-5(4H)-yl]prop-2-en-1-one
1-[2-(4-chloro-2-fluorophenyl)-3-(3-fluoro-1H-pyrrolo[2,3-b]pyridin-4-yl)-6,7-dihydropyrazolo[1,5-a]pyrazin-5(4H)-yl]prop-2-en-1-one
1-[2-(4-chloro-2-fluorophenyl)-3-[2-(methylamino)pyridin-4-yl]-6,7-dihydropyrazolo[1,5-a]pyrazin-5(4H)-yl]prop-2-en-1-one
1-[3-(2-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-[3-(3-cyclopropylpyridin-4-yl)-2-[4-(trifluoromethyl)phenyl]-6,7-dihydropyrazolo[1,5-a]pyrazin-5(4H)-yl]prop-2-en-1-one
1-[3-(2,3-dimethyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
(2E)-1-[2-(4-chloro-2-fluorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one
(2E)-4-(dimethylamino)-1-[3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
(2E)-4-(dimethylamino)-1-[2-(3-fluoro-4-methoxyphenyl)-3-(3-methyl-1H-pyrrolo[2,3-b]pyridin-4-yl)-6,7-dihydropyrazolo[1,5-a]pyrazin-5(4H)-yl]but-2-en-1-one
(2E)-4-(dimethylamino)-1-[2-(4-methoxyphenyl)-3-(3-methyl-1H-pyrrolo[2,3-b]pyridin-4-yl)-6,7-dihydropyrazolo[1,5-a]pyrazin-5(4H)-yl]but-2-en-1-one
3-{5-[(2E)-4-(dimethylamino)but-2-enoyl]-3-(3-methyl-1H-pyrrolo[2,3-b]pyridin-4-yl)-4,5,6,7-tetrahydropyrazolo[1,5-a]pyrazin-2-yl}benzonitrile
2-[(dimethylamino)methyl]-1-[2-(4-fluorophenyl)-3-(pyridin-4-yl)-6,7-dihydropyrazolo[1,5-a]pyrazin-5(4H)-yl]prop-2-en-1-one
(2E)-1-[2-(4-fluorophenyl)-3-(pyridin-4-yl)-6,7-dihydropyrazolo[1,5-a]pyrazin-5(4H)-yl]-4-(morpholin-4-yl) but-2-en-1-one
1-[2-(4-fluorophenyl)-3-(pyridin-4-yl)-6,7-dihydropyrazolo[1,5-a]pyrazin-5(4H)-yl]-2-[(morpholin-4-yl)methyl]prop-2-en-1-one
4-(dimethylamino)-1-[2-(4-fluorophenyl)-3-(pyridin-4-yl)-6,7-dihydropyrazolo[1,5-a]pyrazin-5(4H)-yl]but-2-yn-1-one
(2E)-1-[2-(4-fluorophenyl)-3-(pyridin-4-yl)-6,7-dihydropyrazolo[1,5-a]pyrazin-5(4H)-yl]-4-methoxybut-2-en-1-one
(2E)-4,4,4-trifluoro-1-[2-(4-fluorophenyl)-3-(pyridin-4-yl)-6,7-dihydropyrazolo[1,5-a]pyrazin-5(4H)-yl]but-2-en-1-one
1-[2-(4-fluorophenyl)-3-(pyridin-4-yl)-6,7-dihydropyrazolo[1,5-a]pyrazin-5(4H)-yl]-4-(morpholin-4-yl) but-2-yn-1-one
(2E)-4-(diethylamino)-1-[2-(4-fluorophenyl)-3-(pyridin-4-yl)-6,7-dihydropyrazolo[1,5-a]pyrazin-5(4H)-yl]but-2-en-1-one
(2E)-1-[2-(4-fluorophenyl)-3-(pyridin-4-yl)-6,7-dihydropyrazolo[1,5-a]pyrazin-5(4H)-yl]-4-hydroxybut-2-en-1-one
(2E)-4-[ethyl(methyl)amino]-1-[2-(4-fluorophenyl)-3-(pyridin-4-yl)-6,7-dihydropyrazolo[1,5-a]pyrazin-5(4H)-yl]but-2-en-1-one
1-[2-(4-chloro-2-fluorophenyl)-3-(pyridin-4-yl)-6,7-dihydropyrazolo[1,5-a]pyrazin-5(4H)-yl]-4-(dimethylamino)but-2-yn-1-one
(2E)-1-[2-(4-chloro-2-fluorophenyl)-3-(pyridin-4-yl)-6,7-dihydropyrazolo[1,5-a]pyrazin-5(4H)-yl]-4-fluorobut-2-en-1-one
(2E)-1-[2-(4-chloro-2-fluorophenyl)-3-(pyridin-4-yl)-6,7-dihydropyrazolo[1,5-a]pyrazin-5(4H)-yl]-4,4-difluorobut-2-en-1-one
(2E)-3-chloro-1-[2-(4-chloro-2-fluorophenyl)-3-(pyridin-4-yl)-6,7-dihydropyrazolo[1,5-a]pyrazin-5(4H)-yl]prop-2-en-1-one
[2-(4-chloro-2-fluorophenyl)-3-(pyridin-4-yl)-6,7-dihydropyrazolo[1,5-a]pyrazin-5(4H)-yl](1-methyl-2,5-dihydro-1H-pyrrol-3-yl) methanone
(2E)-4-(azetidin-1-yl)-1-[2-(4-chloro-2-fluorophenyl)-3-(pyridin-4-yl)-6,7-dihydropyrazolo[1,5-a]pyrazin-5(4H)-yl]but-2-en-1-one
(2E)-4-fluoro-1-[2-(4-fluorophenyl)-3-(pyridin-4-yl)-6,7-dihydropyrazolo[1,5-a]pyrazin-5(4H)-yl]but-2-en-1-one
(2E)-1-[2-(4-fluorophenyl)-3-(pyridin-4-yl)-6,7-dihydropyrazolo[1,5-a]pyrazin-5(4H)-yl]-4-(pyrrolidin-1-yl) but-2-en-1-one
(2E)-1-[2-(4-fluorophenyl)-3-(pyridin-4-yl)-6,7-dihydropyrazolo[1,5-a]pyrazin-5(4H)-yl]-4-(methylamino)but-2-en-1-one
(2E)-4-(ethylamino)-1-[2-(4-fluorophenyl)-3-(pyridin-4-yl)-6,7-dihydropyrazolo[1,5-a]pyrazin-5(4H)-yl]but-2-en-1-one
(2E)-1-[2-(4-fluorophenyl)-3-(pyridin-4-yl)-6,7-dihydropyrazolo[1,5-a]pyrazin-5(4H)-yl]-4-(4-methylpiperazin-1-yl) but-2-en-1-one
(2E)-1-[2-(4-fluorophenyl)-3-(pyridin-4-yl)-6,7-dihydropyrazolo[1,5-a]pyrazin-5(4H)-yl]-4-[(3S)-3-methoxypyrrolidin-1-yl]but-2-en-1-one
(2E)-4-(3,3-difluoroazetidin-1-yl)-1-[2-(4-fluorophenyl)-3-(pyridin-4-yl)-6,7-dihydropyrazolo[1,5-a]pyrazin-5(4H)-yl]but-2-en-1-one
1-[2-(4-chloro-2-fluorophenyl)-3-(pyridin-4-yl)-6,7-dihydropyrazolo[1,5-a]pyrazin-5(4H)-yl]-4-(morpholin-4-yl) but-2-yn-1-one
tert-butyl(2RS)-2-(3-[2-(4-fluorophenyl)-3-(pyridin-4-yl)-6,7-dihydropyrazolo[1,5-a]pyrazin-5(4H)-yl]-3-oxoprop-1-yn-1-yl}pyrrolidine-1-carboxylate
1-[2-(4-fluorophenyl)-3-(pyridin-4-yl)-6,7-dihydropyrazolo[1,5-a]pyrazin-5(4H)-yl]-3-[(2RS)-pyrrolidin-2-yl]prop-2-yn-1-one
3-(1-aminocyclopropyl)-1-[2-(4-fluorophenyl)-3-(pyridin-4-yl)-6,7-dihydropyrazolo[1,5-a]pyrazin-5(4H)-yl]prop-2-yn-1-one tert-butyl {4-[2-(4-fluorophenyl)-3-(pyridin-4-yl)-6,7-dihydropyrazolo[1,5-a]pyrazin-5(4H)-yl]-4-oxobut-2-yn-1-yl}carbamate
4-amino-1-[2-(4-fluorophenyl)-3-(pyridin-4-yl)-6,7-dihydropyrazolo[1,5-a]pyrazin-5(4H)-yl]but-2-yn-1-one
1-[2-(4-chloro-2-fluoroanilino)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(3-fluoro-4-methoxyanilino)-3-(3-methyl-1H-pyrrolo[2,3-b]pyridin-4-yl)-6,7-dihydropyrazolo[1,5-a]pyrazin-5(4H)-yl]prop-2-en-1-one
1-[3-(3-methyl-1H-pyrrolo[2,3-b]pyridin-4-yl)-2-[4-(trifluoromethyl) anilino]-6,7-dihydropyrazolo[1,5-a]pyrazin-5(4H)-yl]prop-2-en-1-one
1-[2-(4-chloro-2-fluoroanilino)-3-(pyridin-4-yl)-6,7-dihydropyrazolo[1,5-a]pyrazin-5(4H)-yl]prop-2-en-1-one
1-[2-(3-fluoro-4-methoxyanilino)-3-(pyridin-4-yl)-6,7-dihydropyrazolo[1,5-a]pyrazin-5(4H)-yl]prop-2-en-1-one
1-[3-(pyridin-4-yl)-2-[4-(trifluoromethyl) anilino]-6,7-dihydropyrazolo[1,5-a]pyrazin-5(4H)-yl]prop-2-en-1-one
(2E)-4-(dimethylamino)-1-[2-(4-fluorophenyl)-3-(1H-pyrrolo[2,3-b]pyridin-4-yl)-6,7-dihydropyrazolo[1,5-a]pyrazin-5(4H)-yl]but-2-en-1-one
(2E)-4-(dimethylamino)-1-[2-(2-fluoro-4-methylphenyl)-3-(1H-pyrrolo[2,3-b]pyridin-4-yl)-6,7-dihydropyrazolo[1,5-a]pyrazin-5(4H)-yl]but-2-en-1-one
3-[(6R)-5-[(2E)-4-(dimethylamino)but-2-enoyl]-6-methyl-3-(pyridin-4-yl)-4,5,6,7-tetrahydropyrazolo[1,5-a]pyrazin-2-yl]benzonitrile
3-[(6S)-5-[(2E)-4-(dimethylamino)but-2-enoyl]-6-methyl-3-(pyridin-4-yl)-4,5,6,7-tetrahydropyrazolo[1,5-a]pyrazin-2-yl]benzonitrile
1-[2-(2-fluoro-4-methylphenyl)-3-(1H-pyrrolo[2,3-b]pyridin-4-yl)-6,7-dihydropyrazolo[1,5-a]pyrazin-5(4H)-yl]prop-2-en-1-one
(2E)-1-[2-(4-chloro-2-fluorophenyl)-3-(1H-pyrrolo[2,3-b]pyridin-4-yl)-6,7-dihydropyrazolo[1,5-a]pyrazin-5(4H)-yl]-4-(dimethylamino)but-2-en-1-one
1-[2-(4-chloro-2-fluorophenyl)-3-(1H-pyrrolo[2,3-b]pyridin-4-yl)-6,7-dihydropyrazolo[1,5-a]pyrazin-5(4H)-yl]prop-2-en-1-one
1-[2-(4-fluoro-1-benzofuran-7-yl)-3-(pyridin-4-yl)-6,7-dihydropyrazolo[1,5-a]pyrazin-5(4H)-yl]prop-2-en-1-one
1-[2-(4-fluoronaphthalen-1-yl)-3-(pyridin-4-yl)-6,7-dihydropyrazolo[1,5-a]pyrazin-5(4H)-yl]prop-2-en-1-one
1-[2-(1-benzofuran-7-yl)-3-(pyridin-4-yl)-6,7-dihydropyrazolo[1,5-a]pyrazin-5(4H)-yl]prop-2-en-1-one
1-[2-(3-methyl-1H-indol-6-yl)-3-(pyridin-4-yl)-6,7-dihydropyrazolo[1,5-a]pyrazin-5(4H)-yl]prop-2-en-1-one
1-[2-(3-fluoronaphthalen-2-yl)-3-(pyridin-4-yl)-6,7-dihydropyrazolo[1,5-a]pyrazin-5(4H)-yl]prop-2-en-1-one
1-[2-(6-fluoro-1-benzofuran-5-yl)-3-(pyridin-4-yl)-6,7-dihydropyrazolo[1,5-a]pyrazin-5(4H)-yl]prop-2-en-1-one
1-[2-(1-fluoronaphthalen-2-yl)-3-(pyridin-4-yl)-6,7-dihydropyrazolo[1,5-a]pyrazin-5(4H)-yl]prop-2-en-1-one
1-[2-(2-hydroxy-4-methylphenyl)-3-(pyridin-4-yl)-6,7-dihydropyrazolo[1,5-a]pyrazin-5(4H)-yl]prop-2-en-1-one
1-[2-(2-fluoro-4-methylphenyl)-3-(pyridin-4-yl)-6,7-dihydropyrazolo[1,5-a]pyrazin-5(4H)-yl]prop-2-en-1-one
1-[2-(7-fluoro-1H-indol-6-yl)-3-(pyridin-4-yl)-6,7-dihydropyrazolo[1,5-a]pyrazin-5(4H)-yl]prop-2-en-1-one
1-[2-(4-chloro-2-methylphenyl)-3-(pyridin-4-yl)-6,7-dihydropyrazolo[1,5-a]pyrazin-5(4H)-yl]prop-2-en-1-one
1-[2-(2,4-dichlorophenyl)-3-(pyridin-4-yl)-6,7-dihydropyrazolo[1,5-a]pyrazin-5(4H)-yl]prop-2-en-1-one
1-[2-(4-chloro-2-methoxyphenyl)-3-(pyridin-4-yl)-6,7-dihydropyrazolo[1,5-a]pyrazin-5(4H)-yl]prop-2-en-1-one
2-chloro-5-[5-(prop-2-enoyl)-3-(pyridin-4-yl)-4,5,6,7-tetrahydropyrazolo[1,5-a]pyrazin-2-yl]benzonitrile
2-fluoro-5-[5-(prop-2-enoyl)-3-(pyridin-4-yl)-4,5,6,7-tetrahydropyrazolo[1,5-a]pyrazin-2-yl]benzonitrile
1-[(6R)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one
2-fluoro-3-[5-(prop-2-enoyl)-3-(pyridin-4-yl)-4,5,6,7-tetrahydropyrazolo[1,5-a]pyrazin-2-yl]benzonitrile
1-{2-[3-(hydroxymethyl)phenyl]-3-(pyridin-4-yl)-6,7-dihydropyrazolo[1,5-a]pyrazin-5(4H)-yl}prop-2-en-1-one
1-[(6S)-2-(4-chlorophenyl)-6-methyl-3-(pyridin-4-yl)-6,7-dihydropyrazolo[1,5-a]pyrazin-5(4H)-yl]prop-2-en-1-one
1-[(6R)-2-(4-chlorophenyl)-6-methyl-3-(pyridin-4-yl)-6,7-dihydropyrazolo[1,5-a]pyrazin-5(4H)-yl]prop-2-en-1-one
1-[(6S)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one
3-[(6R)-6-methyl-5-(prop-2-enoyl)-3-(pyridin-4-yl)-4,5,6,7-tetrahydropyrazolo[1,5-a]pyrazin-2-yl]benzonitrile
3-[(6S)-6-methyl-5-(prop-2-enoyl)-3-(pyridin-4-yl)-4,5,6,7-tetrahydropyrazolo[1,5-a]pyrazin-2-yl]benzonitrile
1-{2-[4-(difluoromethoxy)phenyl]-3-(pyridin-4-yl)-6,7-dihydropyrazolo[1,5-a]pyrazin-5(4H)-yl}prop-2-en-1-one
1-[3-(pyridin-4-yl)-2-[4-(trifluoromethoxy)phenyl]-6,7-dihydropyrazolo[1,5-a]pyrazin-5(4H)-yl]prop-2-en-1-one
1-[2-(4-ethoxyphenyl)-3-(pyridin-4-yl)-6,7-dihydropyrazolo[1,5-a]pyrazin-5(4H)-yl]prop-2-en-1-one
1-{2-[4-(difluoromethyl)phenyl]-3-(pyridin-4-yl)-6,7-dihydropyrazolo[1,5-a]pyrazin-5(4H)-yl}prop-2-en-1-one
1-[2-(5-chloro-2-fluorophenyl)-3-(pyridin-4-yl)-6,7-dihydropyrazolo[1,5-a]pyrazin-5(4H)-yl]prop-2-en-1-one
1-[2-(3-chlorophenyl)-3-(pyridin-4-yl)-6,7-dihydropyrazolo[1,5-a]pyrazin-5(4H)-yl]prop-2-en-1-one
1-{2-[3-fluoro-4-(trifluoromethyl)phenyl]-3-(pyridin-4-yl)-6,7-dihydropyrazolo[1,5-a]pyrazin-5(4H)-yl}prop-2-en-1-one
1-{2-[2-fluoro-4-(trifluoromethyl)phenyl]-3-(pyridin-4-yl)-6,7-dihydropyrazolo[1,5-a]pyrazin-5(4H)-yl}prop-2-en-1-one
1-[2-(2-fluorophenyl)-3-(pyridin-4-yl)-6,7-dihydropyrazolo[1,5-a]pyrazin-5(4H)-yl]prop-2-en-1-one
1-[2-(2-chlorophenyl)-3-(pyridin-4-yl)-6,7-dihydropyrazolo[1,5-a]pyrazin-5(4H)-yl]prop-2-en-1-one
1-[2-(4-methylphenyl)-3-(pyridin-4-yl)-6,7-dihydropyrazolo[1,5-a]pyrazin-5(4H)-yl]prop-2-en-1-one
1-[2-(3-methylphenyl)-3-(pyridin-4-yl)-6,7-dihydropyrazolo[1,5-a]pyrazin-5(4H)-yl]prop-2-en-1-one
1-[2-(4-ethylphenyl)-3-(pyridin-4-yl)-6,7-dihydropyrazolo[1,5-a]pyrazin-5(4H)-yl]prop-2-en-1-one
3-[5-(prop-2-enoyl)-3-(pyridin-4-yl)-4,5,6,7-tetrahydropyrazolo[1,5-a]pyrazin-2-yl]benzonitrile
1-[2-(2-fluoro-4-methoxyphenyl)-3-(pyridin-4-yl)-6,7-dihydropyrazolo[1,5-a]pyrazin-5(4H)-yl]prop-2-en-1-one
1-[3-(pyridin-4-yl)-2-{4-[(trifluoromethyl)sulfanyl]phenyl}-6,7-dihydropyrazolo[1,5-a]pyrazin-5(4H)-yl]prop-2-en-1-one
(2E)-1-{2-[4-(difluoromethoxy)phenyl]-3-(pyridin-4-yl)-6,7-dihydropyrazolo[1,5-a]pyrazin-5(4H)-yl}-4-(dimethylamino)but-2-en-1-one
(2E)-4-(dimethylamino)-1-[3-(pyridin-4-yl)-2-[4-(trifluoromethoxy)phenyl]-6,7-dihydropyrazolo[1,5-a]pyrazin-5(4H)-yl]but-2-en-1-one
(2E)-4-(dimethylamino)-1-[2-(4-ethynylphenyl)-3-(pyridin-4-yl)-6,7-dihydropyrazolo[1,5-a]pyrazin-5(4H)-yl]but-2-en-1-one
(2E)-4-(dimethylamino)-1-[2-(4-ethoxyphenyl)-3-(pyridin-4-yl)-6,7-dihydropyrazolo[1,5-a]pyrazin-5(4H)-yl]but-2-en-1-one
(2E)-1-{2-[4-(difluoromethyl)phenyl]-3-(pyridin-4-yl)-6,7-dihydropyrazolo[1,5-a]pyrazin-5(4H)-yl}-4-(dimethylamino)but-2-en-1-one
(2E)-1-[2-(5-chloro-2-fluorophenyl)-3-(pyridin-4-yl)-6,7-dihydropyrazolo[1,5-a]pyrazin-5(4H)-yl]-4-(dimethylamino)but-2-en-1-one
(2E)-1-[2-(3-chlorophenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-{2-[3-fluoro-4-(trifluoromethyl)phenyl]-3-(pyridin-4-yl)-6,7-dihydropyrazolo[1,5-a]pyrazin-5(4H)-yl}but-2-en-1-one
(2E)-4-(dimethylamino)-1-(2-[2-fluoro-4-(trifluoromethyl)phenyl]-3-(pyridin-4-yl)-6,7-dihydropyrazolo[1,5-a]pyrazin-5(4H)-yl}but-2-en-1-one
(2E)-4-(dimethylamino)-1-[2-(2-fluorophenyl)-3-(pyridin-4-yl)-6,7-dihydropyrazolo[1,5-a]pyrazin-5(4H)-yl]but-2-en-1-one
(2E)-1-[2-(2-chlorophenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(3-methoxyphenyl)-3-(pyridin-4-yl)-6,7-dihydropyrazolo[1,5-a]pyrazin-5(4H)-yl]but-2-en-1-one
(2E)-4-(dimethylamino)-1-[2-(4-methylphenyl)-3-(pyridin-4-yl)-6,7-dihydropyrazolo[1,5-a]pyrazin-5(4H)-yl]but-2-en-1-one
(2E)-4-(dimethylamino)-1-[2-(3-methylphenyl)-3-(pyridin-4-yl)-6,7-dihydropyrazolo[1,5-a]pyrazin-5(4H)-yl]but-2-en-1-one
(2E)-4-(dimethylamino)-1-[2-(4-ethylphenyl)-3-(pyridin-4-yl)-6,7-dihydropyrazolo[1,5-a]pyrazin-5(4H)-yl]but-2-en-1-one
3-{5-[(2E)-4-(dimethylamino)but-2-enoyl]-3-(pyridin-4-yl)-4,5,6,7-tetrahydropyrazolo[1,5-a]pyrazin-2-yl}benzonitrile
(2E)-4-(dimethylamino)-1-[2-(2-fluoro-4-methoxyphenyl)-3-(pyridin-4-yl)-6,7-dihydropyrazolo[1,5-a]pyrazin-5(4H)-yl]but-2-en-1-one
(2E)-4-(dimethylamino)-1-[3-(pyridin-4-yl)-2-{4-[(trifluoromethyl) sulfanyl]phenyl}-6,7-dihydropyrazolo[1,5-a]pyrazin-5(4H)-yl]but-2-en-1-one
(2E)-4-(azetidin-1-yl)-1-[2-(4-chlorophenyl)-3-(pyridin-4-yl)-6,7-dihydropyrazolo[1,5-a]pyrazin-5(4H)-yl]but-2-en-1-one
(2E)-1-[2-(4-chlorophenyl)-3-(pyridin-4-yl)-6,7-dihydropyrazolo[1,5-a]pyrazin-5(4H)-yl]-4-(piperidin-1-yl) but-2-en-1-one
(2E)-1-[2-(4-chlorophenyl)-3-(pyridin-4-yl)-6,7-dihydropyrazolo[1,5-a]pyrazin-5(4H)-yl]-4-(4-methylpiperazin-1-yl) but-2-en-1-one
(E)-1-(2-(4-chlorophenyl)-3-(pyridin-4-yl)-6,7-dihydropyrazolo[1,5-a]pyrazin-5(4H)-yl)-4-morpholinobut-2-en-1-one
(2E)-1-[2-(4-chlorophenyl)-3-(pyridin-4-yl)-6,7-dihydropyrazolo[1,5-a]pyrazin-5(4H)-yl]-4-[isopropyl(methyl)amino]but-2-en-1-one
(2E)-4-(tert-butylamino)-1-[2-(4-chlorophenyl)-3-(pyridin-4-yl)-6,7-dihydropyrazolo[1,5-a]pyrazin-5(4H)-yl]but-2-en-1-one
(2E)-1-[2-(4-chlorophenyl)-3-(pyridin-4-yl)-6,7-dihydropyrazolo[1,5-a]pyrazin-5(4H)-yl]-4-(4-methoxypiperidin-1-yl) but-2-en-1-one
(2E)-1-[2-(4-chlorophenyl)-3-(pyridin-4-yl)-6,7-dihydropyrazolo[1,5-a]pyrazin-5(4H)-yl]-4-[(2-methoxyethyl)(methyl)amino]but-2-en-1-one
(2E)-1-[2-(4-chlorophenyl)-3-(pyridin-4-yl)-6,7-dihydropyrazolo[1,5-a]pyrazin-5(4H)-yl]-4-[(2-hydroxyethyl)(methyl)amino]but-2-en-1-one
(2E)-1-[2-(4-chlorophenyl)-3-(pyridin-4-yl)-6,7-dihydropyrazolo[1,5-a]pyrazin-5(4H)-yl]-4-(pyrrolidin-1-yl) but-2-en-1-one
(2E)-1-[2-(4-chlorophenyl)-3-(pyridin-4-yl)-6,7-dihydropyrazolo[1,5-a]pyrazin-5(4H)-yl]-4-(3-fluoroazetidin-1-yl) but-2-en-1-one
1-[2-(4-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-(2-(2-fluoro-4-(trifluoromethyl)phenyl)-3-(3-methyl-1H-pyrrolo[2,3-b]pyridin-4-yl)-6,7-dihydropyrazolo[1,5-a]pyrazin-5(4H)-yl) prop-2-en-1-one
1-[2-(4-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl)]prop-2-en-1-one
1-[2-(3-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) prop-2-en-1-one
1-[2-(2-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]prop-2-en-1-one
4-[3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
(2E)-1-[2-(4-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one
(2E)-1-[2-(3-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one
(E)-4-(dimethylamino)-1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) but-2-en-1-one
(2E)-1-[2-(1-benzofuran-7-yl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one formic acid
(2E)-4-(dimethylamino)-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]but-2-en-1-one
1-[3-(pyridin-4-yl)-2-[6-(trifluoromethyl)pyridin-3-yl]-6,7-dihydropyrazolo[1,5-a]pyrazin-5(4H)-yl]prop-2-en-1-one
1-[2-phenyl-3-(pyridin-4-yl)-6,7-dihydropyrazolo[1,5-a]pyrazin-5(4H)-yl]prop-2-en-1-one
1-[2-(4-chloro-3-ethylphenyl)-3-(pyridin-4-yl)-6,7-dihydropyrazolo[1,5-a]pyrazin-5(4H)-yl]prop-2-en-1-one
1-{2-[4-chloro-2-(hydroxymethyl)phenyl]-3-(pyridin-4-yl)-6,7-dihydropyrazolo[1,5-a]pyrazin-5(4H)-yl}prop-2-en-1-one
1-[2-(1-benzothiophen-5-yl)-3-(pyridin-4-yl)-6,7-dihydropyrazolo[1,5-a]pyrazin-5(4H)-yl]prop-2-en-1-one
1-[2-(1H-indol-5-yl)-3-(pyridin-4-yl)-6,7-dihydropyrazolo[1,5-a]pyrazin-5(4H)-yl]prop-2-en-1-one
1-[2-(1-benzofuran-5-yl)-3-(pyridin-4-yl)-6,7-dihydropyrazolo[1,5-a]pyrazin-5(4H)-yl]prop-2-en-1-one
1-[2-(1H-indol-6-yl)-3-(pyridin-4-yl)-6,7-dihydropyrazolo[1,5-a]pyrazin-5(4H)-yl]prop-2-en-1-one
(2E)-1-[2-(1-benzothiophen-5-yl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(1H-indol-5-yl)-3-(pyridin-4-yl)-6,7-dihydropyrazolo[1,5-a]pyrazin-5(4H)-yl]but-2-en-1-one
(2E)-1-[2-(1H-benzotriazol-6-yl)-3-(pyridin-4-yl)-6,7-dihydropyrazolo[1,5-a]pyrazin-5(4H)-yl]-4-(dimethylamino)but-2-en-1-one
(2E)-1-[2-(1-benzofuran-5-yl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(1H-indol-6-yl)-3-(pyridin-4-yl)-6,7-dihydropyrazolo[1,5-a]pyrazin-5(4H)-yl]but-2-en-1-one rac-(R,E)-4-(dimethylamino)-1-(2-(4-fluorophenyl)-7-(hydroxymethyl)-3-(pyridin-4-yl)-6,7-dihydropyrazolo[1,5-a]pyrazin-5(4H)-yl) but-2-en-1-one
(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazine-7-carboxamide
1-[(7RS)-2-(4-fluorophenyl)-7-(hydroxymethyl)-3-(pyridin-4-yl)-6,7-dihydropyrazolo[1,5-a]pyrazin-5(4H)-yl]prop-2-en-1-one
(7RS)-5-acryloyl-2-(4-fluorophenyl)-3-(pyridin-4-yl)-4,5,6,7-tetrahydropyrazolo[1,5-a]pyrazine-7-carboxamide
5-acryloyl-2-(4-fluorophenyl)-N-methyl-3-(pyridin-4-yl)-4,5,6,7-tetrahydropyrazolo[1,5-a]pyrazine-7-carboxamide
5-acryloyl-2-(4-fluorophenyl)-N,N-dimethyl-3-(pyridin-4-yl)-4,5,6,7-tetrahydropyrazolo[1,5-a]pyrazine-7-carboxamide
1-[2-(4-fluorophenyl)-7,7-dimethyl-3-(pyridin-4-yl)-6,7-dihydropyrazolo[1,5-a]pyrazin-5(4H)-yl]prop-2-en-1-one
1-[2-(4-chloro-3-methylphenyl)-7,7-dimethyl-3-(pyridin-4-yl)-6,7-dihydropyrazolo[1,5-a]pyrazin-5(4H)-yl]prop-2-en-1-one
1-[2-(4-chlorophenyl)-7,7-dimethyl-3-(pyridin-4-yl)-6,7-dihydropyrazolo[1,5-a]pyrazin-5(4H)-yl]prop-2-en-1-one
1-[2-(4-fluorophenyl)-6,6-dimethyl-3-(pyridin-4-yl)-6,7-dihydropyrazolo[1,5-a]pyrazin-5(4H)-yl]prop-2-en-1-one
1-[2-(4-chlorophenyl)-6,6-dimethyl-3-(pyridin-4-yl)-6,7-dihydropyrazolo[1,5-a]pyrazin-5(4H)-yl]prop-2-en-1-one
1-[2-(4-chloro-3-methylphenyl)-6,6-dimethyl-3-(pyridin-4-yl)-6,7-dihydropyrazolo[1,5-a]pyrazin-5(4H)-yl]prop-2-en-1-one
(2E)-4-(dimethylamino)-1-[2-(4-fluorophenyl)-7,7-dimethyl-3-(pyridin-4-yl)-6,7-dihydropyrazolo[1,5-a]pyrazin-5(4H)-yl]but-2-en-1-one
(2E)-1-[2-(4-chloro-3-methylphenyl)-7,7-dimethyl-3-(pyridin-4-yl)-6,7-dihydropyrazolo[1,5-a]pyrazin-5(4H)-yl]-4-(dimethylamino)but-2-en-1-one
1-[(2E)-4-(dimethylamino)but-2-enoyl]-3′-phenyl-2′-(pyridin-4-yl)-5′,6′-dihydrospiro[piperidine-4,7′-pyrrolo[3,2-c]pyridin]-4′ (1′H)-one
(2E)-4-(dimethylamino)-1-[2-(4-fluorophenyl)-6,6-dimethyl-3-(pyridin-4-yl)-6,7-dihydropyrazolo[1,5-a]pyrazin-5(4H)-yl]but-2-en-1-one
(2E)-1-[2-(4-chlorophenyl)-6,6-dimethyl-3-(pyridin-4-yl)-6,7-dihydropyrazolo[1,5-a]pyrazin-5(4H)-yl]-4-(dimethylamino)but-2-en-1-one
(2E)-1-[2-(4-chloro-3-methylphenyl)-6,6-dimethyl-3-(pyridin-4-yl)-6,7-dihydropyrazolo[1,5-a]pyrazin-5(4H)-yl]-4-(dimethylamino)but-2-en-1-one
rac-(R,E)-4-(dimethylamino)-1-(2-(4-fluorophenyl)-7-(2-hydroxyethyl)-3-(pyridin-4-yl)-6,7-dihydropyrazolo[1,5-a]pyrazin-5(4H)-yl) but-2-en-1-one
2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]acetamide
2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N-methylacetamide
2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N,N-dimethylacetamide
1-(2-(4-fluorophenyl)-7-(2-hydroxyethyl)-3-(pyridin-4-yl)-6,7-dihydropyrazolo[1,5-a]pyrazin-5(4H)-yl) prop-2-en-1-one
2-(5-acryloyl-2-(4-fluorophenyl)-3-(pyridin-4-yl)-4,5,6,7-tetrahydropyrazolo[1,5-a]pyrazin-7-yl) acetamide
2-(5-acryloyl-2-(4-fluorophenyl)-3-(pyridin-4-yl)-4,5,6,7-tetrahydropyrazolo[1,5-a]pyrazin-7-yl)-N-methylacetamide
2-(5-acryloyl-2-(4-fluorophenyl)-3-(pyridin-4-yl)-4,5,6,7-tetrahydropyrazolo[1,5-a]pyrazin-7-yl)-N,N-dimethylacetamide
1-(2-(4-chloro-2-fluorophenyl)-6-methyl-3-(pyridin-4-yl)-6,7-dihydropyrazolo[1,5-a]pyrazin-5(4H)-yl) prop-2-en-1-one
1-[(6RS)-2-(4-methoxyphenyl)-6-methyl-3-(pyridin-4-yl)-6,7-dihydropyrazolo[1,5-a]pyrazin-5(4H)-yl]prop-2-en-1-one
1-[(RS)-2-(2-fluoro-1-benzothiophen-6-yl)-6-methyl-3-(pyridin-4-yl)-6,7-dihydropyrazolo[1,5-a]pyrazin-5(4H)-yl]prop-2-en-1-one
1-[(6RS)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one
1-[(RS)-2-(2-fluoro-4-methoxyphenyl)-6-methyl-3-(pyridin-4-yl)-6,7-dihydropyrazolo[1,5-a]pyrazin-5(4H)-yl]prop-2-en-1-one
1-[(RS)-6-methyl-2-(naphthalen-2-yl)-3-(pyridin-4-yl)-6,7-dihydropyrazolo[1,5-a]pyrazin-5(4H)-yl]prop-2-en-1-one
1-[(RS)-2-(2-chloro-1-benzothiophen-6-yl)-6-methyl-3-(pyridin-4-yl)-6,7-dihydropyrazolo[1,5-a]pyrazin-5(4H)-yl]prop-2-en-1-one
1-[(6RS)-2-(1-benzothiophen-6-yl)-6-methyl-3-(pyridin-4-yl)-6,7-dihydropyrazolo[1,5-a]pyrazin-5(4H)-yl]prop-2-en-1-one
(2E)-1-[(RS)-2-(4-chloro-2-fluorophenyl)-6-methyl-3-(pyridin-4-yl)-6,7-dihydropyrazolo[1,5-a]pyrazin-5(4H)-yl]-4-(dimethylamino)but-2-en-1-one
(2E)-1-[(RS)-2-(1-benzothiophen-6-yl)-6-methyl-3-(pyridin-4-yl)-6,7-dihydropyrazolo[1,5-a]pyrazin-5(4H)-yl]-4-(dimethylamino)but-2-en-1-one
1-[(RS)-2-(4-chloro-2-fluorophenyl)-6-methyl-3-(pyridin-4-yl)-6,7-dihydropyrazolo[1,5-a]pyrazin-5(4H)-yl]-2-fluoroprop-2-en-1-one
1-[(RS)-2-(1-benzothiophen-6-yl)-6-methyl-3-(pyridin-4-yl)-6,7-dihydropyrazolo[1,5-a]pyrazin-5(4H)-yl]-2-fluoroprop-2-en-1-one
1-[(RS)-2-(4-fluorophenyl)-4-methyl-3-(pyridin-4-yl)-6,7-dihydropyrazolo[1,5-a]pyrazin-5(4H)-yl]prop-2-en-1-one
1-[(RS)-2-(4-chloro-2-fluorophenyl)-4-methyl-3-(pyridin-4-yl)-6,7-dihydropyrazolo[1,5-a]pyrazin-5(4H)-yl]prop-2-en-1-one
1-[(RS)-2-(4-methoxyphenyl)-4-methyl-3-(pyridin-4-yl)-6,7-dihydropyrazolo[1,5-a]pyrazin-5(4H)-yl]prop-2-en-1-one
1-[(RS)-2-(1-benzothiophen-5-yl)-4-methyl-3-(pyridin-4-yl)-6,7-dihydropyrazolo[1,5-a]pyrazin-5(4H)-yl]prop-2-en-1-one
1-[(RS)-2-(1H-indol-6-yl)-4-methyl-3-(pyridin-4-yl)-6,7-dihydropyrazolo[1,5-a]pyrazin-5(4H)-yl]prop-2-en-1-one
(2E)-1-[(RS)-2-(4-chloro-2-fluorophenyl)-4-methyl-3-(pyridin-4-yl)-6,7-dihydropyrazolo[1,5-a]pyrazin-5(4H)-yl]-4-(dimethylamino)but-2-en-1-one
(2E)-1-[(6S)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one
(2E)-1-[(6R)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one
1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one
1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one
1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one
1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one
4-(dimethylamino)-1-[4-methyl-3-(1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one
(2E)-4-(dimethylamino)-1-[(6S)-6-methyl-3-(3-methyl-1H-pyrrolo[2,3-b]pyridin-4-yl)-2~ [4-(trifluoromethyl)phenyl]-6,7-dihydropyrazolo[1,5-a]pyrazin-5(4H)-yl]but-2-en-1-one
4-(dimethylamino)-1-[2-(4-fluorophenyl)-4-methyl-3-(1H-pyrrolo[2,3-b]pyridin-4-yl)-6,7-dihydropyrazolo[1,5-a]pyrazin-5(4H)-yl]but-2-en-1-one
(E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one
(2E)-4-(dimethylamino)-1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one
1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one
1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one
1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one
1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one
1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one
(E)-4-(dimethylamino)-1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one
(E)-4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one
(2E)-4-(dimethylamino)-1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
(E)-4-(dimethylamino)-1-(2-(4-fluorophenyl)-4-methyl-3-(1H-pyrrolo[2,3-b]pyridin-4-yl)-6,7-dihydropyrazolo[1,5-a]pyrazin-5(4H)-yl) but-2-en-1-one
(E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one
(2E)-4-(dimethylamino)-1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one
1-[(6S)-2-(4-chloro-2-fluorophenyl)-6-methyl-3-(pyridin-4-yl)-6,7-dihydropyrazolo[1,5-a]pyrazin-5(4H)-yl]prop-2-en-1-one
1-[(6R)-2-(4-chloro-2-fluorophenyl)-6-methyl-3-(pyridin-4-yl)-6,7-dihydropyrazolo[1,5-a]pyrazin-5(4H)-yl]prop-2-en-1-one
1-[2-anilino-3-(pyridin-4-yl)-6,7-dihydropyrazolo[1,5-a]pyrazin-5(4H)-yl]prop-2-en-1-one
(2E)-1-[2-anilino-3-(pyridin-4-yl)-6,7-dihydropyrazolo[1,5-a]pyrazin-5(4H)-yl]-4-(dimethylamino)but-2-en-1-one
1-[2-(4-methoxyphenyl)-7-methyl-3-(pyridin-4-yl)-6,7-dihydropyrazolo[1,5-a]pyrazin-5(4H)-yl]prop-2-en-1-one
1-[(6S)-2-(1-benzothiophen-5-yl)-6-methyl-3-(pyridin-4-yl)-6,7-dihydropyrazolo[1,5-a]pyrazin-5(4H)-yl]prop-2-en-1-one
1-[(6R)-2-(1-benzothiophen-5-yl)-6-methyl-3-(pyridin-4-yl)-6,7-dihydropyrazolo[1,5-a]pyrazin-5(4H)-yl]prop-2-en-1-one
1-[2-(4-chloro-2-fluorophenyl)-4-methyl-3-(pyridin-4-yl)-6,7-dihydropyrazolo[1,5-a]pyrazin-5(4H)-yl]prop-2-en-1-one
1-[(7RS)-2-(1-benzothiophen-5-yl)-7-methyl-3-(pyridin-4-yl)-6,7-dihydropyrazolo[1,5-a]pyrazin-5(4H)-yl]prop-2-en-1-one
1-[(7RS)-2-(4-chlorophenyl)-7-methyl-3-(pyridin-4-yl)-6,7-dihydropyrazolo[1,5-a]pyrazin-5(4H)-yl]prop-2-en-1-one
1-[2-(4-chloro-2-fluorophenyl)-7-methyl-3-(pyridin-4-yl)-6,7-dihydropyrazolo[1,5-a]pyrazin-5(4H)-yl]prop-2-en-1-one
3-[(6S)-6-methyl-3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
3-[(6R)-6-methyl-3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
(2E)-1-[(2-(4-chloro-2-fluorophenyl)-4-methyl-3-(pyridin-4-yl)-6,7-dihydropyrazolo[1,5-a]pyrazin-5(4H)-yl]-4-(dimethylamino)but-2-en-1-one
(2E)-1-[2-(4-chloro-2-fluorophenyl)-7-methyl-3-(pyridin-4-yl)-6,7-dihydropyrazolo[1,5-a]pyrazin-5(4H)-yl]-4-(dimethylamino)but-2-en-1-one
3-[(6S)-5-[(2E)-4-(dimethylamino)but-2-enoyl]-6-methyl-3-(3-methyl-1H-pyrrolo[2,3-b]pyridin-4-yl)-4,5,6,7-tetrahydropyrazolo[1,5-a]pyrazin-2-yl]benzonitrile
3-[(6R)-5-[(2E)-4-(dimethylamino)but-2-enoyl]-6-methyl-3-(3-methyl-1H-pyrrolo[2,3-b]pyridin-4-yl)-4,5,6,7-tetrahydropyrazolo[1,5-a]pyrazin-2-yl]benzonitrile
1-[(6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one
1-[(6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one or an N-oxide, a salt, a tautomer, a rotamer, or a stereoisomer of said compound, or a salt of said N-oxide, tautomer, rotamer, or stereoisomer.

A further aspect of the invention relates to compounds of formula (I), which are present as their salts, such as pharmaceutically acceptable salts.

It is to be understood that the present invention relates to any sub-combination within any embodiment or aspect of the present invention of compounds of formula (I), supra.

More particularly still, the present invention covers compounds of formula (I) which are disclosed in the Example section of this text, infra.

In accordance with another aspect, the present invention covers methods of preparing compounds of the present invention, said methods comprising the steps as described in the Experimental Section herein.

Another embodiment of the invention are compounds according as disclosed in the Claims section or disclosed analogs of the exemplified compounds and subcombinations thereof.

### Definitions

It is to be understood that embodiments disclosed herein are not meant to be understood as individual embodiments which would not relate to one another. Features discussed with one embodiment or aspect of the invention are meant to be disclosed also in connection with other embodiments or aspects of the invention shown herein. If, in one case, a specific feature is not disclosed with one embodiment or aspect of the invention, but with another, the skilled person would understand that does not necessarily mean that said feature is not meant to be disclosed with said other embodiment or aspect of the invention. The skilled person would understand that it is the gist of this application to disclose said feature also for the other embodiment or aspect of the invention, but that just for purposes of clarity and to keep the length of this specification manageable. For example, it is to be understood that all aspects, embodiments, pharmaceutical compositions, combinations, uses and/or methods of the present invention defined herein for the compounds of formula (I) also relate to more specific embodiments of the compounds of formula (I), such as, but not limited to, the compounds of formula (Ia) and vice-versa, for example.

It is further to be understood that the content of the documents referred to herein is incorporated by reference in their entirety, namely when e.g. a method is discussed details of which are described in said document. This approach serves to keep the length of this specification manageable.

The term “comprising” when used in the specification includes “consisting of”.

If it is referred to “as mentioned above” or “mentioned above”, “supra” within the description it is referred to any of the disclosures made within the specification in any of the preceding pages.

If it is referred to “as mentioned herein”, “described herein”, “provided herein,” or “as mentioned in the present text,” or “stated herein” within the description it is referred to any of the disclosures made within the specification in any of the preceding or subsequent pages.

By “subject” is meant a mammal, including, but not limited to, a human or non-human mammal, such as a bovine, equine, canine, ovine, or feline.

“Suitable” within the sense of the invention means chemically possible to be made by methods within the knowledge of a skilled person.

The terms as mentioned in the present text may have the following meanings:

The term “C₃-C₆-cycloalkyl” means a saturated, monovalent, monocyclic hydrocarbon ring which contains 3, 4, 5, or 6 carbon atoms. Said C₃-C₆-cycloalkyl group is for example, a cyclopropyl, cyclobutyl, cyclopentyl, or cyclohexyl group.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₅, C₅-C₆, particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

As used herein, the term “one or more”, e.g. in the definition of the substituents of the compounds of the formulae of the present invention, is understood as meaning “one, two, three, four, five, etc, particularly one, two, three or four, more particularly one, two or three, even more particularly one or two”.

The compounds of formula (I) may exist as isotopic variants. The invention therefore includes one or more isotopic variant(s) of the compounds of formula (I), particularly deuterium-containing compounds of formula (I).

The term “isotopic variant” of a compound or a reagent is defined as a compound exhibiting an unnatural proportion of one or more of the isotopes that constitute such a compound.

The term “isotopic variant of the compound of formula (I)” is defined as a compound of formula (I) exhibiting an unnatural proportion of one or more of the isotopes that constitute such a compound.

The expression “unnatural proportion” is to be understood as meaning a proportion of such isotope which is higher than its natural abundance. The natural abundances of isotopes to be applied in this context are described in “Isotopic Compositions of the Elements 1997”, Pure Appl. Chem., 70 (1), 217-235, 1998. Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of formula (I) in one embodiment contain deuterium (“deuterium-containing compounds of formula (I)”). Isotopic variants of the compounds of formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of formula (I). These isotopic variants of the compounds of formula (I) are useful for in vivo imaging applications. Deuterium-containing and ¹³C-containing compounds of formula (I) can be used in mass spectrometry analyses (H. J. Leis et al., Curr. Org. Chem., 1998, 2, 131) in the context of preclinical or clinical studies.

Isotopic variants of the compounds of formula (I) can generally be prepared by methods known to a person skilled in the art, such as those described in the schemes and/or examples herein, by substituting a reagent for an isotopic variant of said reagent, in one embodiment for a deuterium-containing reagent. Depending on the desired sites of deuteration, in some cases deuterium from D2O can be incorporated either directly into the compounds or into reagents that are useful for synthesizing such compounds (Esaki et al., Tetrahedron, 2006, 62, 10954; Esaki et al., Chem. Eur. J., 2007, 13, 4052). Deuterium gas is also a useful reagent for incorporating deuterium into molecules. Catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron, 2011, 52, 3865) is a rapid route for incorporation of deuterium. Metal catalysts (i.e. Pd, Pt, and Rh) in the presence of deuterium gas can be used to directly exchange deuterium for hydrogen in functional groups containing hydrocarbons (J. G. Atkinson et al., U.S. Pat. No. 3,966,781). A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16 (4), 683-688, 1993; J. Atzrodt et al., Angew. Chem., Int. Ed. 2007, 46, 7744; K. Matoishi et al., J. Chem. Soc, Chem. Commun. 2000, 1519-1520; K. Kassahun et al., WO2012/112363.

The term “deuterium-containing compound of formula (I)” is defined as a compound of formula (I), in which one or more hydrogen atom(s) is/are replaced by one or more deuterium atom(s) and in which the abundance of deuterium at each deuterated position of the compound of formula (I) is higher than the natural abundance of deuterium, which is about 0.015%. Particularly, in a deuterium-containing compound of formula (I) the abundance of deuterium at each deuterated position of the compound of formula (I) is higher than 10%, 20%, 30%, 40%, 50%, 60%, 70% or 80%, in one embodiment higher than 90%, 95%, 96% or 97%, in other embodiments higher than 98% or 99% at said position(s). It is understood that the abundance of deuterium at each deuterated position is independent of the abundance of deuterium at other deuterated position(s).

The selective incorporation of one or more deuterium atom(s) into a compound of formula (I) may alter the physicochemical properties (such as for example acidity [A. Streitwieser et al., J. Am. Chem. Soc., 1963, 85, 2759; C. L. Perrin, et al., J. Am. Chem. Soc., 2007, 129, 4490], basicity [C. L. Perrin, et al., J. Am. Chem. Soc., 2003, 125, 15008; C. L. Perrin in Advances in Physical Organic Chemistry, 44, 144; C. L. Perrin et al., J. Am. Chem. Soc., 2005, 127, 9641], lipophilicity [B. Testa et al., Int. J. Pharm., 1984, 19 (3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (D. J. Kushner et al., Can. J. Physiol. Pharmacol., 1999, 77, 79; A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Uetrecht et al., Chemical Research in Toxicology, 2008, 21, 9, 1862; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. Indiplon (A. J. Morales et al., Abstract 285, The 15th North American Meeting of the International Society of Xenobiotics, San Diego, CA, Oct. 12-16, 2008), ML-337 (C. J. Wenthur et al., J. Med. Chem., 2013, 56, 5208), and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch. Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of formula (I) may have multiple potential sites of attack for metabolism. To optimize the above-described effects on physicochemical properties and metabolic profile, deuterium-containing compounds of formula (I) having a certain pattern of one or more deuterium-hydrogen exchange(s) can be selected. Particularly, the deuterium atom(s) of deuterium-containing compound(s) of formula (I) is/are attached to a carbon atom and/or is/are located at those positions of the compound of formula (I), which are sites of attack for metabolizing enzymes such as e.g. cytochrome P450.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or(S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations (including enantiomers and diastereomers), are included within the scope of the present invention.

Typically, compounds of the present disclosure are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

In certain compounds of the present invention, optionally asymmetry may be present due to restricted rotation about a given bond, for example, the central bond adjoining two substituted aromatic rings of the specified compounds. Particularly, dependent on the sterical demands of the substituents in position R¹, R^{4a}, R^{4b}, R^{10a}, and R^{10d}, compounds of the present invention can exist as atropisomers, as shown in FIG. 1. Atropisomers represent a subclass of conformers which arise from restricted rotation around a single bond. The conformers (called atropisomers) can be isolated as separated species (IUPAC Gold book, http://goldbook.jupac.org/A00511.html; Pure and Appl. Chem., 2009, 68, 2193-2222). This induced chirality belongs to the axial type of chirality. Hence, compounds featuring said atropisomerism and an additional asymmetric centre can exist as diasteromeric mixtures as described supra.

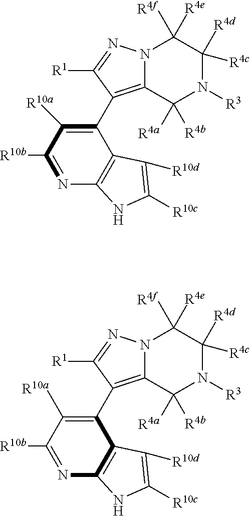

FIG. 1: Atropisomerism examples of compounds of general formula (I)

Several related compounds with the related type of atropisomerism have been already described as observed in the literature (G. Bringmann et al., Chem. Rev., 2011, 111, 563-639). For examples of atropisomers in drug discovery, see the mini-review from J. Clayden, S. R. Laplante et al.: Angew. Chem. Int. Ed. 2009, 48, 6398-6401 and also: S. R. LaPlante, P. J. Edwards et al., J. Med. Chem. 2011, 54, 7005-7022.

Further, the compounds of the present invention may optionally contain one or more asymmetric centres, depending upon the location and nature of the various substituents desired. It is possible that one or more asymmetric carbon atoms are present in the (R) or(S) configuration, which can result in racemic mixtures in the case of a single asymmetric centre, and in diastereomeric mixtures in the case of multiple elements of asymmetry, such as axial chirality and asymmetric centres.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art, examples of which can be found in the experimental section. If the atropisomers were separated, said atropisomers are being referred to as “atrop 1” (for atropisomer 1) and “atrop 2” (for atropisomer 2), subsequent to the respective compound name. Names without any such indication but still naming a compound showing atropisomerism is to be understood to include both atropisomers which were not separated.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

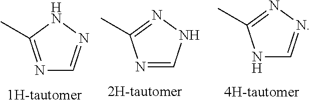

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-). mono-, sesqui-, di-, tri-, tetra-, penta-etc, solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and jodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

The salts include water-insoluble and, particularly, water-soluble salts.

Furthermore, derivatives of the compounds of formula (I) and the salts thereof which are converted into a compound of formula (I) or a salt thereof in a biological system (bioprecursors or pro-drugs) are covered by the invention. Said biological system is e.g. a mammalian organism, particularly a human subject. The bioprecursor is, for example, converted into the compound of formula (I) or a salt thereof by metabolic processes.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₃ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl, 1,3-dioxolen-2-onylmethyl esters, e.g. 5-methyl-1,3-dioxolen-2-onylmethyl, and C₁-C₃-alkoxycarbonyloxyethyl esters, e.g. 1-methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorphs, in any ratio.

In the context of the properties of the compounds of the present invention the term “pharmacokinetic profile” means one single parameter or a combination thereof including permeability, bioavailability, exposure, and pharmacodynamic parameters such as duration, or magnitude of pharmacological effect, as measured in a suitable experiment. Compounds with improved pharmacokinetic profiles can, for example, be used in lower doses to achieve the same effect, may achieve a longer duration of action, or a may achieve a combination of both effects.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered. Any such combination of a compound of formula (I) of the present invention with an anti-cancer agent as defined below is an embodiment of the invention.

The term “(chemotherapeutic) anti-cancer agents” relates to any agent that reduces the survival or proliferation of a cancer cell, and includes but is not limited to 131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, poziotinib, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, streptozocin, sunitinib, talaporfin, tamibarotene, tamoxifen, tapentadol, stanozolol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

By “Epidermal Growth Factor Receptor (EGFR) Polypeptide” is meant a polypeptide having at least about 95% amino acid sequence identity to the sequence provided at UniProt Accession No. P00533-1 or a fragment thereof. In some embodiments, the EGFR fragment binds an EFGR ligand and/or has kinase activity. Mutant EGFR polypeptides include those having an insertion between, for example, amino acids V769 and D770 or between D770 and N771. In other embodiments, the amino acid sequence identity is 96, 97, 98, 99, or 100% to UniProt Accession No. P00533-1.

An exemplary full length sequence of human EGFR, which indicates V769, D770, and N771 in bold, is provided at UniProt Accession No. P00533-1, which is reproduced below:

```
                10 20 30 40
MRPSGTAGAA LLALLAALCP ASRALEEKKV CQGTSNKLTQ
                50 60 70 80
LGTFEDHFLS LQRMFNNCEV VIGNLEITYV QRNYDLSFLK
                90 100 110 120
TIQEVAGYVL IALNTVERIP LENLQIIRGN MYYENSYALA
              130 140 150 160
VLSNYDANKT GLKELPMRNL QEILHGAVRF SNNPALCNVE
              170 180 190 200
SIQWRDIVSS DFLSNMSMDF QNHLGSCQKC DPSCPNGSCW
              210 220 230 240
GAGEENCQKL TKIICAQQCS GRCRGKSPSD CCHNQCAAGC
              250 260 270 280
TGPRESDCLV CRKFRDEATC KDTCPPLMLY NPTTYQMDVN
              290 300 310 320
PEGKYSFGAT CVKKCPRNYV VTDHGSCVRA CGADSYEMEE
              330 340 350 360
DGVRKCKKCE GPCRKVCNGI GIGEFKDSLS INATNIKHFK
              370 380 390 400
NCTSISGDLH ILPVAFRGDS FTHTPPLDPQ ELDILKTVKE
              410 420 430 440
ITGFLLIQAW PENRTDLHAF ENLEIIRGRT KQHGQFSLAV
              450 460 470 480
VSLNITSLGL RSLKEISDGD VIISGNKNIC YANTINWKKL
              490 500 510 520
FGTSGQKTKI ISNRGENSCK ATGQVCHALC SPEGCWGPEP
              530 540 550 560
RDCVSCRNVS RGRECVDKCN LLEGEPREFV ENSECIQCHP
              570 580 590 600
ECLPQAMNIT CTGRGPDNCI QCAHYIDGPH CVKTCPAGVM
              610 620 630 640
GENNTLVWKY ADAGHVCHLC HPNCTYGCTG PGLEGCPTNG
              650 660 670 680
PKIPSIATGM VGALLLLLVV ALGIGLFMRR RHIVRKRTLR
              690 700 710 720
RLLQERELVE PLTPSGEAPN QALLRILKET EFKKIKVLGS
              730 740 750 760
GAFGTVYKGL WIPEGEKVKI PVAIKELREA TSPKANKEIL
              770 780 790 800
DEAYVMASVD NPHVCRLIGI CLTSTVQLIT QLMPFGCLLD
              810 820 830 840
YVREHKDNIG SQYLLNWCVQ IAKGMNYLED RRLVHRDLAA
              850 860 870 880
RNVLVKTPQH VKITDFGLAK LLGAEEKEYH AEGGKVPIKW
              890 900 910 920
MALESILHRI YTHQSDVWSY GVTVWELMTF GSKPYDGIPA
              930 940 950 960
SEISSILEKG ERLPQPPICT IDVYMIMVKC WMIDADSRPK
              970 980 990 1000
FRELIIEFSK MARDPQRYLV IQGDERMHLP SPTDSNFYRA
            1010 1020 1030 1040
LMDEEDMDDV VDADEYLIPQ QGFFSSPSTS RTPLLSSLSA
            1050 1060 1070 1080
TSNNSTVACI DRNGLQSCPI KEDSFLQRYS SDPTGALTED
            1090 1100 1110 1120
SIDDTFLPVP EYINQSVPKR PAGSVQNPVY HNQPLNPAPS
            1130 1140 1150 1160
RDPHYQDPHS TAVGNPEYLN TVQPTCVNST FDSPAHWAQK
            1170 1180 1190 1200
GSHQISLDNP DYQQDFFPKE AKPNGIFKGS TAENAEYLRV
            1210
APQSSEFIGA
```

By “Epidermal Growth Factor Receptor (EGFR) Polynucleotide” is meant a nucleic acid molecule encoding an EGFR polypeptide or fragment thereof. An exemplary polynucleotide encoding EGFR is provided at NCBI Reference Sequence: NM_001346897.1, which is reproduced below:

| | |
|---|---|
| 1 | gtccgggcag cccccggcgc agcgcggccg cagcagcctc cgccccccgc acggtgtgag |
| 61 | cgcccgacgc ggccgaggcg gccggagtcc cgagctagcc ccggcggccg ccgccgccca |
| 121 | gaccggacga caggccacct cgtcggcgtc cgcccgagtc cccgcctcgc cgccaacgcc |
| 181 | acaaccaccg cgcacggccc cctgactccg tccagtattg atcgggagag ccggagcgag |
| 241 | ctcttcgggg agcagcgatg cgaccctccg ggacggccgg ggcagcgctc ctggcgctgc |
| 301 | tggctgcgct ctgcccggcg agtcgggctc tggaggaaaa gaaagtttgc caaggcacga |
| 361 | gtaacaagct cacgcagttg ggcacttttg aagatcattt tctcagcctc cagaggatgt |
| 421 | tcaataactg tgaggtggtc cttgggaatt tggaaattac ctatgtgcag aggaattatg |
| 481 | atctttcctt cttaaagacc atccaggagg tggctggtta tgtcctcatt gccctcaaca |
| 541 | cagtggagcg aattcctttg gaaaacctgc agatcatcag aggaaatatg tactacgaaa |
| 601 | attcctatgc cttagcagtc ttatctaact atgatgcaaa taaaaccgga ctgaaggagc |
| 661 | tgcccatgag aaatttacag ggccaaaagt gtgatccaag ctgtcccaat gggagctgct |
| 721 | ggggtgcagg agaggagaac tgccagaaac tgaccaaaat catctgtgcc cagcagtgct |
| 781 | ccgggcgctg ccgtggcaag tcccccagtg actgctgcca caaccagtgt gctgcaggct |
| 841 | gcacaggccc ccgggagagc gactgcctgg tctgccgcaa attccgagac gaagccacgt |
| 901 | gcaaggacac ctgcccccca ctcatgctct acaaccccac cacgtaccag atggatgtga |
| 961 | accccgaggg caaatacagc tttggtgcca cctgcgtgaa gaagtgtccc cgtaattatg |
| 1021 | tggtgacaga tcacggctcg tgcgtccgag cctgtggggc cgacagctat gagatggagg |
| 1081 | aagacggcgt ccgcaagtgt aagaagtgcg aagggccttg ccgcaaagtg tgtaacggaa |
| 1141 | taggtattgg tgaatttaaa gactcactct ccataaatgc tacgaatatt aaacacttca |
| 1201 | aaaactgcac ctccatcagt ggcgatctcc acatcctgcc ggtggcattt aggggtgact |
| 1261 | ccttcacaca tactcctcct ctggatccac aggaactgga tattctgaaa accgtaaagg |
| 1321 | aaatcacagg gtttttgctg attcaggctt ggcctgaaaa caggacggac ctccatgcct |
| 1381 | ttgagaacct agaaatcata cgcggcagga ccaagcaaca tggtcagttt tctcttgcag |
| 1441 | tcgtcagcct gaacataaca tccttgggat tacgctccct caaggagata agtgatggag |
| 1501 | atgtgataat ttcaggaaac aaaaatttgt gctatgcaaa tacaataaac tggaaaaaac |
| 1561 | tgtttgggac ctccggtcag aaaaccaaaa ttataagcaa cagaggtgaa aacagctgca |
| 1621 | aggccacagg ccaggtctgc catgccttgt gctcccccga gggctgctgg ggcccggagc |
| 1681 | ccagggactg cgtctcttgc cggaatgtca gccgaggcag ggaatgcgtg gacaagtgca |
| 1741 | accttctgga gggtgagcca agggagtttg tggagaactc tgagtgcata cagtgccacc |
| 1801 | cagagtgcct gcctcaggcc atgaacatca cctgcacagg acggggacca gacaactgta |
| 1861 | tccagtgtgc ccactacatt gacggccccc actgcgtcaa gacctgcccg gcaggagtca |
| 1921 | tgggagaaaa caacaccctg gtctggaagt acgcagacgc cggccatgtg tgccacctgt |
| 1981 | gccatccaaa ctgcacctac ggatgcactg ggccaggtct tgaaggctgt ccaacgaatg |
| 2041 | ggcctaagat cccgtccatc gccactggga tggtgggggc cctcctcttg ctgctggtgg |
| 2101 | tggccctggg gatcggcctc ttcatgcgaa ggcgccacat cgttcggaag cgcacgctgc |
| 2161 | ggaggctgct gcaggagagg gagcttgtgg agcctcttac acccagtgga gaagctccca |
| 2221 | accaagctct cttgaggatc ttgaaggaaa ctgaattcaa aaagatcaaa gtgctgggct |
| 2281 | ccggtgcgtt cggcacggtg tataagggac tctggatccc agaaggtgag aaagttaaaa |
| 2341 | ttcccgtcgc tatcaaggaa ttaagagaag caacatctcc gaaagccaac aaggaaatcc |
| 2401 | tcgatgaagc ctacgtgatg gccagcgtgg acaaccccca cgtgtgccgc ctgctgggca |
| 2461 | tctgcctcac ctccaccgtg cagctcatca cgcagctcat gcccttcggc tgcctcctgg |
| 2521 | actatgtccg ggaacacaaa gacaatattg gctcccagta cctgctcaac tggtgtgtgc |
| 2581 | agatcgcaaa gggcatgaac tacttggagg accgtcgctt ggtgcaccgc gacctggcag |
| 2641 | ccaggaacgt actggtgaaa acaccgcagc atgtcaagat cacagatttt gggctggcca |
| 2701 | aactgctggg tgcggaagag aaagaatacc atgcagaagg aggcaaagtg cctatcaagt |
| 2761 | ggatggcatt ggaatcaatt ttacacagaa tctataccca ccagagtgat gtctggagct |
| 2821 | acggggtgac tgtttgggag ttgatgacct ttggatccaa gccatatgac ggaatccctg |
| 2881 | ccagcgagat ctcctccatc ctggagaaag gagaacgcct ccctcagcca cccatatgta |
| 2941 | ccatcgatgt ctacatgatc atggtcaagt gctggatgat agacgcagat agtcgcccaa |
| 3001 | agttccgtga gttgatcatc gaattctcca aaatggcccg agacccccag cgctaccttg |
| 3061 | tcattcaggg ggatgaaaga atgcatttgc caagtcctac agactccaac ttctaccgtg |
| 3121 | ccctgatgga tgaagaagac atggacgacg tggtggatgc cgacgagtac ctcatcccac |
| 3181 | agcagggctt cttcagcagc ccctccacgt cacggactcc cctcctgagc tctctgagtg |
| 3241 | caaccagcaa caattccacc gtggcttgca ttgatagaaa tgggctgcaa agctgtccca |
| 3301 | tcaaggaaga cagcttcttg cagcgataca gctcagaccc cacaggcgcc ttgactgagg |
| 3361 | acagcataga cgacaccttc ctcccagtgc ctggtgagtg gcttgtctgg aaacagtcct |
| 3421 | gctcctcaac ctcctcgacc cactcagcag cagccagtct ccagtgtcca agccaggtgc |
| 3481 | tccctccagc atctccagag ggggaaacag tggcagattt gcagacacag tgaagggcgt |
| 3541 | aaggagcaga taaacacatg accgagcctg cacaagctct ttgttgtgtc tggttgtttg |
| 3601 | ctgtacctct gttgtaagaa tgaatctgca aaatttctag cttatgaagc aaatcacgga |
| 3661 | catacacatc tgtgtgtgtg agtgttcatg atgtgtgtac atctgtgtat gtgtgtgtgt |
| 3721 | gtatgtgtgt gtttgtgaca gatttgatcc ctgttctctc tgctggctct atcttgacct |
| 3781 | gtgaaacgta tatttaacta attaaatatt agttaatatt aataaatttt aagctttatc |
| 3841 | cagaaaaaaa aaaaaaaaa |

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The intermediates used for the synthesis of the compounds of claims 1-4 as described below, as well as their use for the synthesis of the compounds of claims 1-4, are one further aspect of the present invention. Preferred intermediates are the Intermediate Examples as disclosed below.

### General Procedures

The compounds according to the invention can be prepared according to the following schemes 1-6.

The schemes and procedures described below illustrate synthetic routes to the compounds of formula (I) of the invention and are not intended to be limiting. It is obvious to the person skilled in the art that the order of transformations as exemplified in the schemes can be modified in various ways. The order of transformations exemplified in the schemes is therefore not intended to be limiting. In addition, interconversion of any of the substituents R¹, R², R³, R⁴ (e.g., R^{4a}, R^{4b}, R^{4c}, R^{4d}, R^{4e}, R^{4f}), R⁵ (e.g., R^{5a}, R^{5b}, R^{5c}, R^{5d}, R^{5e}), R⁶ (e.g., R^{6a}, R^{6b}, R^{6c}, R^{6d}), R⁷ (e.g., R^{7a}, R^{7b}, R^{7c}, R^{7d}), R⁸ (e.g., R^{8a}, R^{8b}, R^{8c}, R^{8d}, R^{8e}, R⁹ (e.g., R^{9a}, R^{9b}, R^{9c}, R^{9d}, R^{9e}), and PG can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art. Specific examples are described in the subsequent paragraphs.

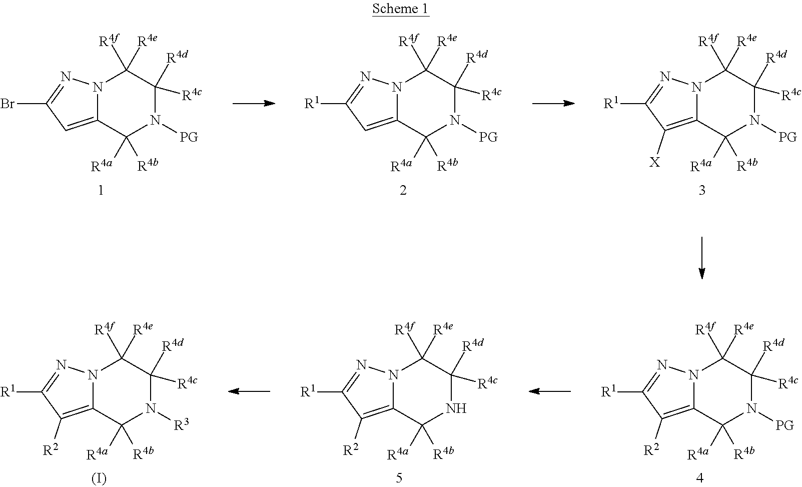

Scheme 1: Route for the preparation of compounds of formula (I), wherein R¹, R², R³, R^{4a}, R^{4b}, R^{4c}, R^{4d}, R^{4e}, and R^{4f} have the meaning as given for formula (I) and PG can be hydrogen or optionally a suitable protecting group, e.g. tert-butoxycarbonyl (Boc), and LG, is a suitable leaving group such as a halide or sulfonate as known to one skilled in the art. Compounds of the formula 1 can be converted to compounds of the formula 2 by reacting for example suitable boronic acids in a Suzuki-type reaction. 2 can be converted to compounds of the formula 3 by halogenation reactions e.g. brominations, with reagents such as N-Bromsuccinimid (NBS) in solvents such as DMF or acetonitrile in a temperature range from −30° C. to the boiling point of the respective solvent. Compounds of formula 3 can be converted to compounds of the type 4 with suitable boronic acids in a Suzuki-type reaction. Compounds of type 5 can be prepared from compounds of the formula 4 by deprotection reactions known to those skilled in the art. Compounds of the formula 5 can be converted to compounds of the formula (I) using various methods. Depending on the nature of R³ corresponding acid chlorides, sulfonylchlorides or other electrophiles can be used under basic conditions in a suitable solvent. If carboxylic acids are employed the corresponding reaction can be facilitated by the use coupling reagents such as HATU, EDC-HOBt or T3P or T4P. Additionally, following the introduction of a substituent at position R³, further synthetic manipulations could be carried out at this position. For instance, elimination of hydrogen chloride, could be used to form an alkene, or transformations such as alkylation's may be undertaken to introduce additional substituents such as alkylamines. Furthermore, the sequence shown here may be altered, for instance formula type 1, could be halogenated, affording a halogenated intermediate, which through a sequence of selective cross coupling reactions could also be used to generate compounds of type 4, as obvious to one skilled in the art.

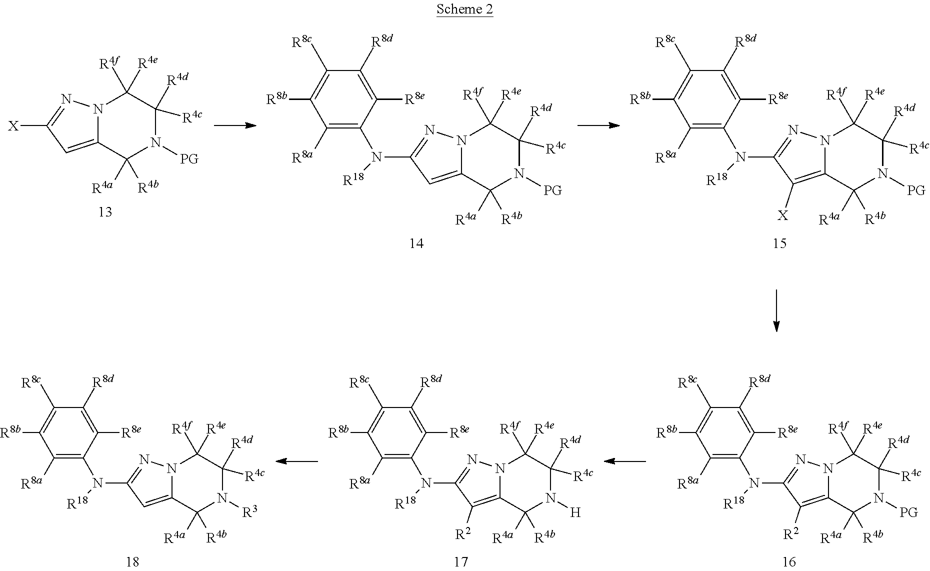

Scheme 2: Route for the preparation of compounds of formula (18), wherein R², R³, R^{4a}, R^{4b}, R^{4c}, R^{4d}, R^{4e}, R^{4f}, R^{5a}, R^{5b}, R^{8c}, R^{8d}, R^{8e}, and R¹⁶ have the meaning as given for formula (I) and PG can be hydrogen or optionally a suitable protecting group, e.g. tert-butoxycarbonyl (Boc). X represents a halogen, preferably Cl, Br or I. Compounds of the formula 13 can be converted to compounds of the formula 14 using various methods which are known to those skilled in the art. These transformations include Buchwald-Hartwig- or Ullmann-reactions with suitable anilins. Compounds of the formula 14 can be converted to compounds of the formula 15 by reactions to introduce halogen known to a person skilled in the art. Introduction of Br is preferred by using N-Bromosuccinimide (NBS) in solvents such as DMF or acetonitrile in a temperature range from −30° C. to the boiling point of the respective solvent. Compounds of the formular 15 can be converted to compounds of the formula 16 by reacting for example suitable boronic acids in a Suzuki-type reaction. In cases in which PG represents a protecting group such as tert-butoxycarbonyl compounds of the formula 17 can be prepared from compounds of the formula 16 by deprotection reactions known to those skilled in the art (or as generally described in the chemical literature such as in Greens Protecting Group in Organic Chemistry). Compounds of the formula 17 can be converted to compounds of the formula 18 using various methods. Depending on the nature of R³ corresponding acid chlorides, sulfonylchlorides or other electrophiles can be used under basic conditions in a suitable solvent. If carboxylic acids are employed the corresponding reaction can be facilitated by the use coupling reagents such as HATU, EDC-HOBt or T3P. Additionally, following the introduction of a substituent at position R³, further synthetic manipulations could be carried out at this position. For instance, elimination of hydrogen chloride, could be used to form an alkene, or transformations such as alkylation's may be undertaken to introduce additional substituents such as alkylamines.

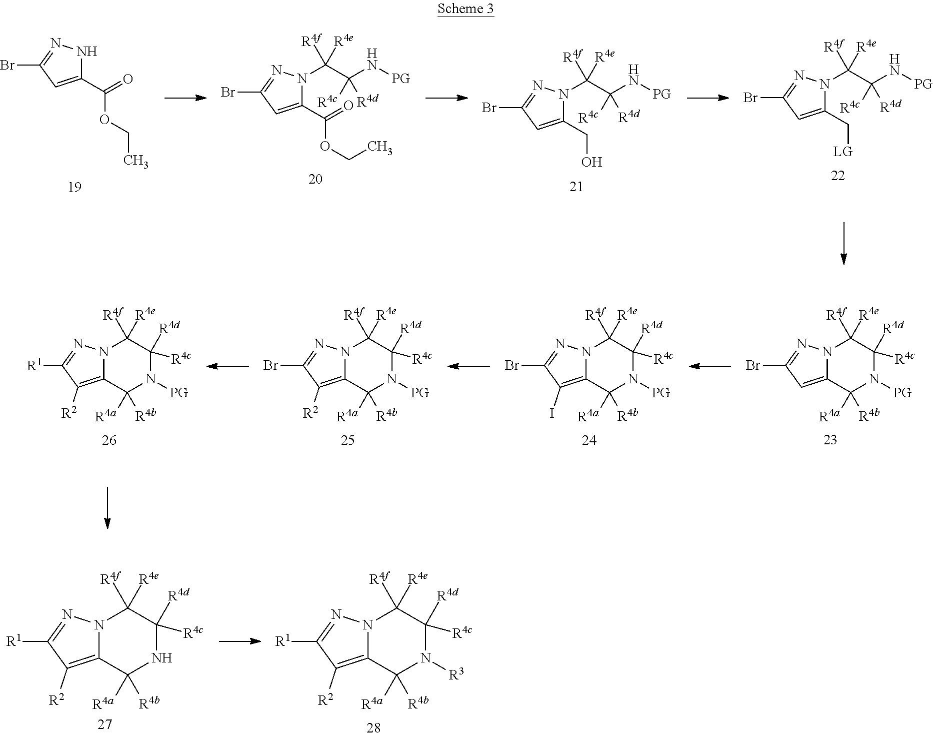

Scheme 3: Route for the preparation of compounds of formula 28, wherein R^{4a} and R^{4b} are defined as hydrogen atoms, and R¹, R², R³, R^{4c}, R^{4d}, R^{4e}, and R^{4f} have the meaning as given for formula (I) and PG can be hydrogen or optionally a suitable protecting group, e.g. tert-butoxycarbonyl (Boc), and LG, is a suitable leaving group such as a halide or sulfonate as known to one skilled in the art. Compounds of the formula 19 can be converted to compounds of the formula 20 using various methods include alkylation with various electrophiles such as halides, or with groups such as mesyl or tosyl, or by direct reaction of alcohols in the presence of triphenylphosphine with azo compounds (Mitsonubu type reactions). 20 can be converted to compounds of the formula 21 by reduction, with reagents such as sodium borohydride. 21 can be converted to compounds of the type 22 by reaction with for instance mesyl chloride, to convert the corresponding alcohol into a suitable leaving group (LG). 22 can be converted to compounds of the type 23 by treatment with a suitable base such as sodium hydride. 23 can be converted to compounds of the type 24 with reagents such as N-iodosuccinimide in a suitable solvent such as DCE, DMF or MeCN in a temperature range from −30° C. to the boiling point of the respective solvent. Compounds of the formular 24 can be converted to compounds of the formula 25 by reacting for example suitable boronic acids in a Suzuki-type reaction. 25 can be converted to compounds like 26 by reacting for example suitable boronic acids in a Suzuki-type reaction. In cases in which PG represents a protecting group such as tert-butoxycarbonyl, compounds of the formula 27 can be prepared from compounds of the formula 26 by deprotection reactions known to those skilled in the art (or as generally described in the chemical literature such as in Greens Protecting Group in Organic Chemistry). Compounds of the formula 27 can be converted to compounds of the formula 28 using various methods. Depending on the nature of R³ corresponding acid chlorides, sulfonylchlorides or other electrophiles can be used under basic conditions in a suitable solvent. If carboxylic acids are employed the corresponding reaction can be facilitated by the use coupling reagents such as HATU, EDC-HOBt or T3P. Additional transformations on the groups R^{4a}, R^{4b}, R^{4c}, R^{4d}, R^{4e} and R^{4f} may be undertaken at suitable stages in the sequence described above, for instance cleavage of protecting groups, reductions or oxidations, and further manipulations such as amide formation, as known to one skilled in the art. Furthermore, the sequence shown here may be altered, for instance formula type 23, could be subjected to an arylation reaction replacing the here depicted bromide, and then subsequently halogenated, and duly subjected to a second arylation reaction, affording compounds of formula type 26, as obvious to one skilled in the art.

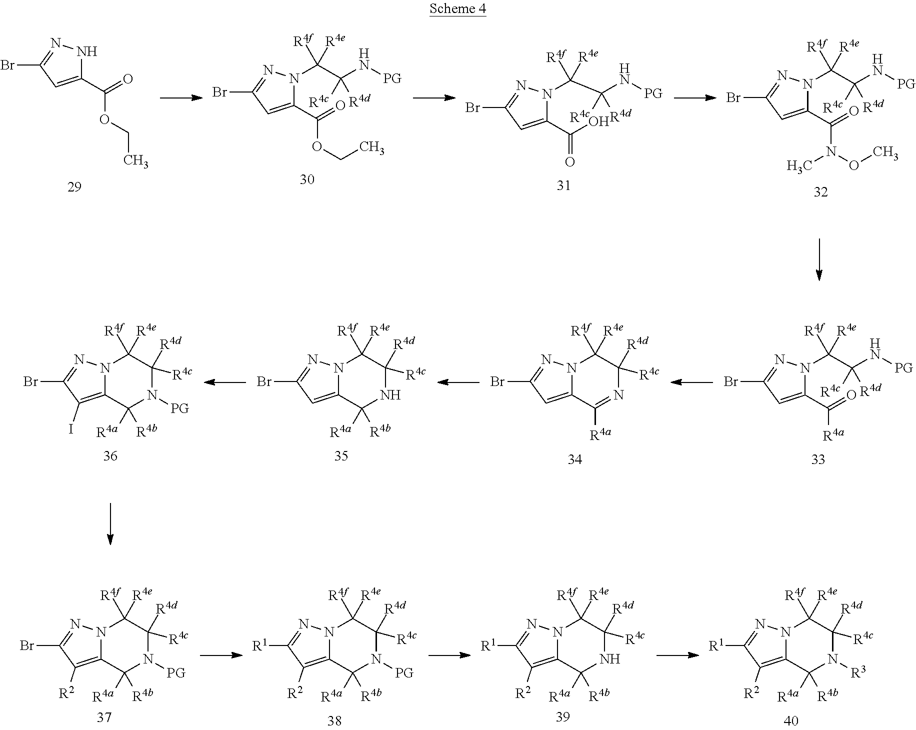

Scheme 4: Route for the preparation of compounds of formula (I), wherein atom and R¹, R², R³, R^{4a}, R^{4b}, R^{4c}, R^{4d}, R^{4e}, and R^{4f} have the meaning as given for formula (I) and PG can be hydrogen or optionally a suitable protecting group, e.g. tert-butoxycarbonyl (Boc). A compound of type 29 (here described as a ethyl ester, but not limited to specifically this ester), could be converted to formula 30 using various methods include alkylation with various electrophiles such as halides, or with groups such as mesyl or tosyl, or by direct reaction of alcohols in the presence of triphenylphosphine with azo compounds (Mitsonubu type reactions). Base or acid catalyzed hydrolysis of the ester produces compounds of formula type 31. Formation of a so called Weinreb-amide by reaction of the corresponding acid with the corresponding amine, facilitated by an amide coupling reagent such as HATU, generates formula 32, which when reacted with a suitable organometallic reagent, for instance a Grignard or alkyl lithium species, as known to one skilled in the art, can be used to generate formula type 33. Deprotection generates an imine of formula 34, which in turn may be reduced with a suitable hydride source such as sodium cyanoborohydride, or subjected to further transformations, as known to one skilled in the art to generate formula type 35. Analogously to Scheme 3, 35 can be converted to compounds of the type 40 by the described transformations.

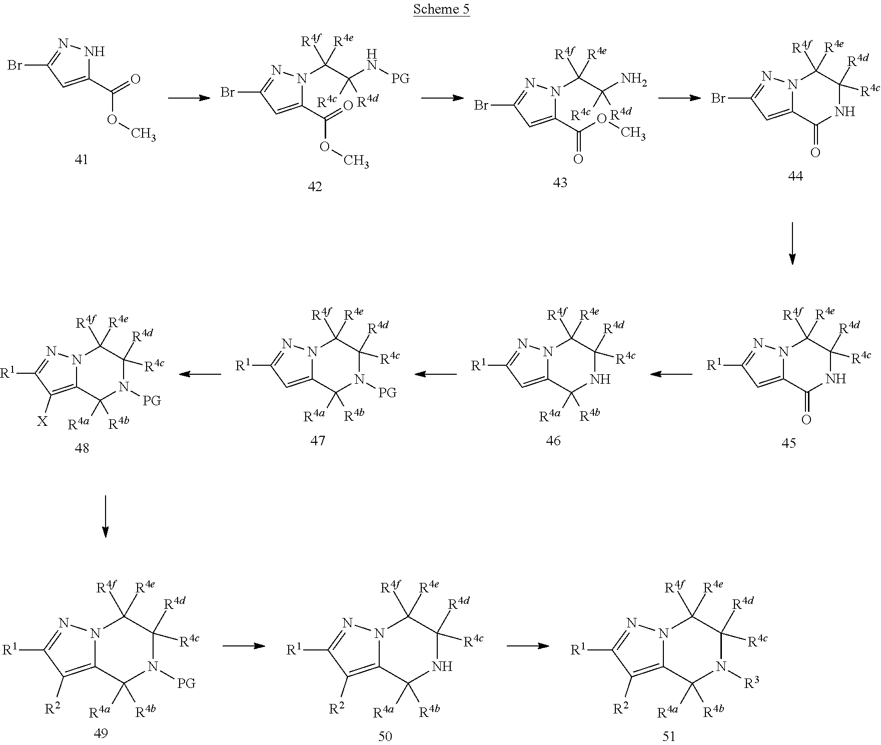

Scheme 5: Route for the preparation of compounds of formula (I), wherein R^{4a}, R^{4b} are hydrogen atoms, and R¹, R², R³, R^{4c}, R^{4d}, R^{4e}, and R^{4f} have the meaning as given for formula (I) and PG can be hydrogen or optionally a suitable protecting group, e.g. tert-butoxycarbonyl (Boc). A compound of type 41 (here described as a methyl ester, but not limited to specifically this ester), could be converted to formula 42 using various methods include alkylation with various electrophiles such as halides, or with groups such as mesyl or tosyl, or by direct reaction of alcohols in the presence of triphenylphosphine with azo compounds (Mitsonubu type reactions). Removal of the protecting group (PG), by for instance treatment with hydrochloric acid (when the protecting group is BOC), yields a compound of formula 43, which can be cyclized to formula 44, by for instance treatment with a base such as triethylamine. Conversion of formula 44 to formula 45 can be accomplished by reaction with for instance a boronic acid in the presence of a suitable catalyst such as palladium ligated by a ligand such as DPPF. Reduction of the lactam with a suitable reagent, such as but not limited to borane dimethyl sulfide, yields formula 46, which can, but not must be suitably protected, as for instance a carbamate, yielding formula type 47. Analogously to Scheme 1, 47 can be converted to compounds of the type 51 by the described transformations.

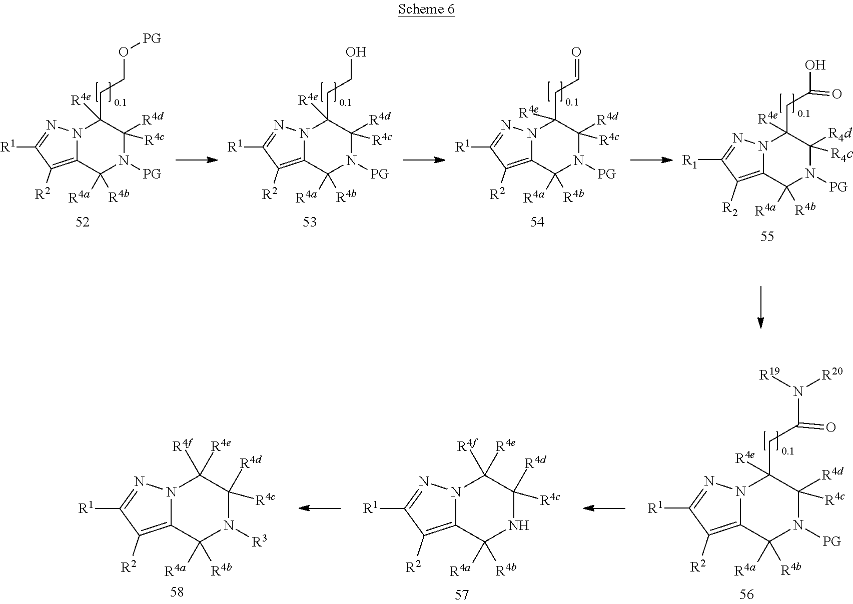

Scheme 6: Route for the preparation of compounds of formula (I), wherein R¹, R², R³, R^{4a}, R^{4b}, R^{4c}, R^{4d}, R^{4e}, and R^{4f} have the meaning as given for formula (I) and PG can be hydrogen or optionally a suitable protecting group, e.g. tert-butoxycarbonyl (Boc) or benzyl, as preferable as known to one skilled in the art. Compounds of formula type 52 may be prepared by the approaches described in Schemes 1-5, as obvious to one skilled in the art. Deprotection of, for instance a benzyl protecting group, using for instance dihydrogen with a suitable catalyst such as palladium, in a suitable solvent, can be performed, yielding compounds of type 53. Oxidation of the corresponding alcohol, using for instance a suitable oxidant, such as for instance Dess-Martin periodane, can be used to produce formula type 54, which can be further oxidized, with for instance sodium chlorite, to the corresponding acid of formula 55. Amide coupling, using for instance HATU and a suitable amine, can be used to produce formula type 56. Deprotection, yielding formula 57, and further transformations, analogously to Schemes 1-5 can be used to produce compounds of formula type 58. Additionally, the order of the sequence could be altered, with for instance formula type 53, could be deprotected, and directly used to produce compounds of formula 58, as known to one skilled in the art.

It is known to the person skilled in the art that, if there are a number of reactive centers on a starting or intermediate compound, it may be necessary to block one or more reactive centers temporarily by protective groups in order to allow a reaction to proceed specifically at the desired reaction center.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC may be applied. The compounds of the present invention which possess a sufficiently basic or acidic functionality, may result as a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of the compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, free acid, solvate, inclusion complex) of a compound of the present invention as isolated and described herein is not necessarily the only form in which said compound can be applied to a biological assay in order to quantify the specific biological activity.

Salts of the compounds of formula (I) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the example section.

Pure diastereomers and pure enantiomers of the compounds and salts according to the invention can be obtained e.g. by asymmetric synthesis, by using chiral starting compounds in synthesis or by splitting up enantiomeric and diasteriomeric mixtures obtained in synthesis.

Enantiomeric and diastereomeric mixtures can be split up into the pure enantiomers and pure diastereomers by methods known to the person skilled in the art. In one embodiment, diastereomeric mixtures are separated by crystallization, in particular fractional crystallization, or chromatography. Enantiomeric mixtures can be separated e.g. by forming diastereomers with a chiral auxillary agent, resolving the diastereomers obtained and removing the chiral auxillary agent. As chiral auxillary agents, for example, chiral acids can be used to separate enantiomeric bases such as e.g. mandelic acid and chiral bases can be used to separate enantiomeric acids by formation of diastereomeric salts. Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxillary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

Compounds featuring atropisomerism and an additional asymmetric centre can exist as diasteromeric mixtures and can be split up into the pure enantiomers and pure diastereomers as described supra.

One preferred aspect of the invention is the process for the preparation of the compounds of claims 1-4 according to the examples as well as the intermediates used for their preparation.

Optionally, compounds of the formula (I) can be converted into their salts, or, optionally, salts of the compounds of the formula (I) can be converted into the free compounds. Corresponding processes are customary for the skilled person.

### Commercial Utility

As mentioned supra, the compounds of the present invention have surprisingly been found to effectively inhibit mutant EGFR in a cell (e.g., a cancer cell) contacted with the compound, thereby inducing cell death (e.g., apoptosis) and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by mutant EGFR, such as, for example, benign and malignant neoplasia, more specifically haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof, especially haematological tumours, solid tumours, and/or metastases of breast, bladder, bone, brain, central and peripheral nervous system, cervix, colon, endocrine glands (e.g., thyroid and adrenal cortex), endocrine tumours, endometrium, esophagus, gastrointestinal tumours, germ cells, kidney, liver, lung, larynx and hypopharynx, mesothelioma, ovary, pancreas, prostate, rectum, renal, small intestine, soft tissue, stomach, skin, testis, ureter, vagina and vulva as well as malignant neoplasias including primary tumours in said organs and corresponding secondary tumours in distant organs (“tumour metastases”). Haematological tumours can, e.g., be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site, as well as AIDS related malignancies.

A further aspect of the invention is the use of the compounds according to formula (I) for the treatment of lung cancer, particularly lung cancer harboring mutant EGFR with exon 20 insertion mutations, more particularly lung cancer harboring V769_770ins ASV and/or D770_N771ins SVD exon 20 insertions, and/or metastases thereof, comprising administering an effective amount of a compound of formula (I).

A further aspect of the invention is the use of the compounds according to formula (1) for the treatment of lung cancer, particularly lung cancer harboring a mutant EGFR with in-frame deletions in exon 19 (such as EGFR E746_A750del) or point mutations in exon 21 (e.g. L858R), and/or metastases thereof.

A further aspect of the invention is the use of the compounds according to formula (I) for the treatment of lung cancer, particularly lung cancer harboring a mutant EGFR with a D770_N771insSVD C797S, E746_A750del C797S, or L858R C797S acquired resistance mutation, and/or metastases thereof.

A further aspect of the invention is the use of the compounds according to formula (I) for the treatment of lung cancer, particularly lung cancer harboring a mutant ERBB2 with exon 20 insertion mutations (such as ERBB2 A775_G776insYVMA), and/or metastases thereof. In accordance with an aspect of the present invention therefore the invention relates to a compound of formula I, or an N-oxide, a salt, a tautomer or a stereoisomer of said compound, or a salt of said N-oxide, tautomer or stereoisomer particularly a pharmaceutically acceptable salt thereof, or a mixture of same, as described and defined herein, for use in the treatment or prophylaxis of a disease, especially for use in the treatment of a disease.

Another particular aspect of the present invention is therefore the use of a compound of formula I, described supra, or a stereoisomer, a tautomer, an N-oxide, a hydrate, a solvate, or a salt thereof, particularly a pharmaceutically acceptable salt thereof, or a mixture of same, for the prophylaxis or treatment of hyperproliferative disorders or disorders responsive to induction of cell death, i.e., apoptosis.

By “hyperproliferative disease” is meant a disease, such as cancer, associated with inappropriately high levels of cell division, inappropriately low levels of apoptosis, or both. The term “inappropriate” within the context of the present invention, in particular in the context of “inappropriate cellular immune responses, or inappropriate cellular inflammatory responses”, as used herein, is to be understood as generally meaning a response, which is less than, or greater than normal, and which is associated with, responsible for, or results in, the pathology of said diseases.

In particular embodiments, the use is in the treatment or prophylaxis of diseases, especially the treatment, wherein the diseases are haematological tumours, solid tumours and/or metastases thereof.

Another aspect is the use of a compound of formula (I) for the prophylaxis and/or treatment of lung cancer, particularly lung cancer harboring mutant EGFR with exon 20 insertion mutations, more particularly lung cancer harboring V769_770ins ASV and/or

D770_N771ins SVD exon 20 insertions, and/or metastases thereof, especially preferred for the treatment thereof.

Another aspect of the present invention is the use of a compound of formula (I) or a stereoisomer, a tautomer, an N-oxide, a hydrate, a solvate, or a salt thereof, particularly a pharmaceutically acceptable salt thereof, or a mixture of same, as described herein, in the manufacture of a medicament for the treatment or prophylaxis of a disease, wherein such disease is a hyperproliferative disorder or a disorder responsive to induction of cell death e.g., apoptosis. In an embodiment the disease is a haematological tumour, a solid tumour and/or metastases thereof. In another embodiment the disease is lung cancer, particularly lung cancer harboring mutant EGFR with exon 20 insertion mutations, more particularly lung cancer harboring V769_770ins ASV and/or D770_N771ins SVD exon 20 insertions, and/or metastases thereof.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce cell death e.g. apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, inverted sinonasal papilloma, inverted sinonasal papilloma-associated sinonasal squamous cell carcinoma, Merkel cell skin cancer, and non-melanoma skin cancer.

Head-and-neck cancers include, but are not limited to laryngeal, hypopharyngeal, nasopharyngeal, oropharyngeal cancer, inverted sinonasal papilloma, inverted sinonasal papilloma-associated sinonasal squamous cell carcinoma, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

The present invention relates to a method of treating cancer in a subject, the method comprising administering to the subject an effective amount of a compound of formula (I) as defined herein.

The present invention relates to a method of treating cancer in a subject, wherein the cancer is or has acquired resistance to an anti-EGF receptor therapy, the method comprising administering to the subject an effective amount of a compound of formula (I) as defined herein.

The present invention relates to a method of enhancing the efficacy of an anti-EGF-receptor therapy, the method comprising administering to the subject an anti-EGF receptor therapy in combination with a a compound of formula (I) as defined herein.

In a further embodiment, the present invention relates to a method of treating cancer in a subject, wherein the cancer is selected from the group consisting of leukemia, myelodysplastic syndrome, malignant lymphoma, head and neck tumours, tumours of the thorax, gastrointestinal tumours, endocrine tumours, mammary and other gynaecological tumours, urological tumours, skin tumours, and sarcomas, the method comprising administering to the subject an effective amount of a compound of formula (I) as defined herein.

In a further embodiment, the present invention relates to a method of treating cancer in a subject, wherein the cancer is selected from the group consisting of inverted sinonasal papilloma or inverted sinonasal papilloma associated sinanonasal squamous cell carcinoma, the method comprising administering to the subject an effective amount of a compound of formula (I) as defined herein.

In a further embodiment, the present invention relates to a method of treating cancer in a subject, wherein the tumour of the thorax is non-small cell lung cancer, the method comprising administering to the subject an effective amount of a compound of formula (I) as defined herein.

In a further embodiment, the present invention relates to a method of treating cancer in a subject, wherein the cancer is lung cancer, particularly lung cancer harboring a mutant EGFR with in-frame deletions in exon 19 (such as EGFR E746_A750del) or point mutations in exon 21 (e.g. L858R), and/or metastases thereof, the method comprising administering to the subject an effective amount of a compound of formula (I) as defined herein.

In a further embodiment, the present invention relates to a a method of treating cancer in a subject, wherein the cancer is lung cancer, particularly lung cancer harboring a mutant EGFR with a D770_N771insSVD C797S, E746_A750del C797S, or L858R C797S acquired resistance mutation, and/or metastases thereof, the method comprising administering to the subject an effective amount of a compound of formula (I) as defined herein.

In a further embodiment, the present invention relates to a a method of treating cancer in a subject, wherein the cancer is lung cancer, particularly lung cancer harboring a mutant ERBB2 with exon 20 insertion mutations (such as ERBB2 A775_G776insYVMA), and/or metastases thereof, the method comprising administering to the subject an effective amount of a compound of formula (I) as defined herein.

The present disclosure is also related to method of selecting a patient for cancer treatment with a compound of formula (I) comprising detecting the presence of a mutation in exon 20 of the gene encoding the EGF-receptor in a biological sample of the subject, thereby determining that the patient should be treated with said compound. In some embodiments, the EGFR comprises aD770_N771insSVD C797S, E746_A750del C797S, or L858R C797S acquired resistance mutation, and/or metastases thereof. In some embodiments, the method of selecting a patient for cancer treatment with a compound of formula (I) may comprise detecting the presence of in-frame deletions in exon 19 or point mutations in exon 21 of the gene encoding EGF-receptor in a biological sample of the subject, thereby determining that the patient should be treated with said compound. For example, the in-frame deletion in exon 19 may be EGFR E746_A750del or the point mutation in exon 21 may be L858R. In some embodiments, the method of selecting a patient for cancer treatment with a compound of formula (I) may comprise detecting the presence of a mutation in exon 20 of the gene encoding ERBB2 in a biological sample of the subject, thereby determining that the patient should be treated with said compound. In some embodiments, the ERBB2 comprises an ERBB2 A775 or_G776insYVMA insertion mutation, and/or metastases thereof. Furthermore, methods of treating a patient with cancer may comprise administering to the subject a compound of formula (I) (e.g., in combination with anti-EGF receptor therapy), wherein the subject is selected for therapy by detecting the presence of a mutation in EGFR in a biological sample of the subject. In some embodiments, the method may comprise obtaining a biological sample from a subject and detecting a mutation in exon 19, 20, or 21 of the gene encoding EGF-receptor in the biological sample obtained from the subject. Detection of the presence of a mutation in exon 20 is within the skill of one of the art.

In embodiments, the disclosure provides a method of treating a selected subject, the method comprising administering to the selected subject a compound described herein, wherein the subject is selected by detecting a mutant EGFR comprising an in-frame deletion in exon 19 (e.g., EGFR E746_A750del) or a point mutations in exon 21 (e.g. L858R).

In some embodiments, the detection of a mutation (e.g., in an EGFR or a mutation in exon 20 of the gene encoding EGFR) may be performed by sequencing (e.g., Sanger, Next Generation Sequencing) or a method selected from the group consisting of immunoblotting, mass spectrometry, immunoprecipitation quantitative PCR, Northern Blot, microarray, enzyme-linked immunosorbent assay (ELISA), in situ hybridization, and combinations thereof.

### Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

### Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death e.g. apoptosis of such cell types.

In various embodiments, the diseases of said method are haematological tumours, solid tumour and/or metastases thereof.

The compounds of the present invention can be used in particular in therapy and prevention i.e. prophylaxis, especially in therapy of tumour growth and metastases, especially in solid tumours of all indications and stages with or without pre-treatment of the tumour growth.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition, disorder, or disease.

Therefore, the present invention includes pharmaceutical compositions that are comprised of a pharmaceutically acceptable carrier or auxiliary and a pharmaceutically effective amount of a compound, or salt thereof, of the present invention.

Another aspect of the invention is a pharmaceutical composition comprising a pharmaceutically effective amount of a compound of formula (I) and a pharmaceutically acceptable auxiliary for the treatment of a disease mentioned supra, especially for the treatment of haematological tumours, solid tumours and/or metastases thereof.

A pharmaceutically acceptable carrier or auxiliary may be a carrier that is non-toxic and innocuous to a patient at concentrations consistent with effective activity of the active ingredient so that any side effects ascribable to the carrier do not vitiate the beneficial effects of the active ingredient. Carriers and auxiliaries are all kinds of additives assisting to the composition to be suitable for administration.

A pharmaceutically effective amount of compound may be that amount which produces a result or exerts the intended influence on the particular condition being treated.

The compounds of the present invention can be administered with pharmaceutically-acceptable carriers or auxiliaries well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing auxiliaries, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration, such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above.

Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in, for example, a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene) s or ethylene oxide or propylene oxide copolymers; and amphoteric detergents, for example, alkyl-beta-aminopropionates, and 2-alkylimidazoline quaternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) in one embodiment of from about 12 to about 17. The quantity of surfactant in such formulation in one embodiment ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for administration, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52 (5), 238-311; Strickley, R. G “Parenteral Formulations of Small Molecule Therapeutics Marketed in the United States (1999)-Part-1” PDA Journal of Pharmaceutical Science & Technology 1999, 53 (6), 324-349; and Nema, S. et al., “Excipients and Their Use in Injectable Products” PDA Journal of Pharmaceutical Science & Technology 1997, 51 (4), 166-171.

Commonly used pharmaceutical ingredients that can be used as appropriate to formulate the composition for its intended route of administration include:
acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃);
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate);
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid);
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate);
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile i.v. solution: A 5 mg/ml solution of the desired compound of this invention can be made using sterile, injectable water, and the pH is adjusted if necessary. The solution is diluted for administration to 1-2 mg/ml with sterile 5% dextrose and is administered as an i.v. infusion over about 60 minutes.

Lyophilised powder for i.v. administration: A sterile preparation can be prepared with (i) 100-1000 mg of the desired compound of this invention as a lyophilised powder, (ii) 32-327 mg/ml sodium citrate, and (ill) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.

Intramuscular suspension: The following solution or suspension can be prepared, for intramuscular injection:
50 mg/ml of the desired, water-insoluble compound of this invention
5 mg/ml sodium carboxymethylcellulose
4 mg/ml TWEEN 80
9 mg/ml sodium chloride
9 mg/ml benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and in particular embodiments from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will in other embodiments be from 0.01 to 200 mg/kg of total body weight. The average daily rectal dosage regimen will in particular embodiments be from 0.01 to 200 mg/kg of total body weight. The average daily vaginal dosage regimen will in other embodiments be from 0.01 to 200 mg/kg of total body weight. The average daily topical dosage regimen will in still other embodiments be from 0.1 to 200 mg administered between one to four times daily. The transdermal concentration will in other embodiments be that required to maintain a daily dose of from 0.01 to 200 mg/kg. The average daily inhalation dosage regimen will in other embodiments be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Combination Therapies

The compounds of this invention can be administered as the sole pharmaceutical agent or in combination with one or more other pharmaceutical agents where the combination causes no unacceptable adverse effects. Those combined pharmaceutical agents can be other agents having antiproliferative effects such as for example for the treatment of haematological tumours, solid tumours and/or metastases thereof and/or agents for the treatment of undesired side effects. The present invention relates also to such combinations.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, especially (chemotherapeutic) anti-cancer agents as defined supra. The combination can be a non-fixed combination or a fixed-dose combination as the case may be.

Methods of testing for a particular pharmacological or pharmaceutical property are well known to persons skilled in the art.

The example testing experiments described herein serve to illustrate the present invention and the invention is not limited to the examples given.

As will be appreciated by persons skilled in the art, the invention is not limited to the particular embodiments described herein, but covers all modifications of said embodiments that are within the spirit and scope of the invention as defined by the appended claims.

The following examples illustrate the invention in greater detail, without restricting it. Further compounds according to the invention, of which the preparation is not explicitly described, can be prepared in an analogous way.

The compounds, which are mentioned in the examples and the salts thereof represent preferred embodiments of the invention as well as a claim covering all subcombinations of the residues of the compound of formula (I) as disclosed by the specific examples.

The term “according to” within the experimental section is used in the sense that the procedure referred to is to be used “analogously to”.

### EXPERIMENTAL SECTION

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations

| Abbreviation | Meaning |
|---|---|
| CAN | acetonitrile |
| AcOH | Acetic acid |
| aq. | aqueous |
| Br | broad signal (NMR) |
| D | doublet (NMR) |
| DAD | Diode Array Detector |
| DIPEA | N,N-diisopropylethylamine |
| DCM | Dichloromethane |
| Dd | doublet of doublet (NMR) |
| DMF | N,N-dimethylformamide |
| DMSO | dimethylsulfoxide |
| EDC•HCl | N-(3-Dimethylaminopropyl)-N′-ethylcarbodiimide |
| | hydrochloride salt |
| ESI | electrospray (ES) ionization |
| ethyl acetate | Ethyl acetate |
| EtOH | Ethanol |
| h, hr (hrs) | hour(s) |
| HATU | (1-[Bis(dimethylamino)methylene]-1H-1,2,3-triazolo[4,5- |
| | b]pyridinium 3-oxide hexafluorophosphate |
| HCl | hydrogen chloride, hydrochloric acid |
| HPLC | high performance liquid chromatography |
| LC-MS | liquid chromatography-mass spectrometry |
| M | multiplet (NMR) |
| MeCN | Acetonitrile |
| MeOH | Methanol |
| Min | minute(s) |
| MS | mass spectrometry |
| NMR | Nuclear Magnetic Resonance spectroscopy: chemical |
| | shifts (δ) are given in ppm. The chemical shifts |
| | were corrected by setting the DMSO signal to 2.50 |
| | ppm using unless otherwise stated. |
| Q | quartet (NMR) |
| Rt or rt | room temperature |
| Rₜ | retention time |
| S | singulet (NMR) |
| sat. | saturated |
| T | triplet (NMR) |
| Td | triplet of doublet (NMR) |
| TEA | triethylamine |
| TFA | Trifluoroacetic acid |
| THF | tetrahydrofuran |
| T3P | propanephosphonic acid anhydride |
| Δ | chemical shift |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be removed by trituration using a suitable solvent. In some cases, the compounds may be purified by chromatography, particularly flash column chromatography, using for example prepacked silica gel cartridges, e.g. Biotage SNAP cartridges KP-Sil® or KP—NH® in combination with a Biotage autopurifier system (SP4® or Isolera Four®) and eluents such as gradients of hexane/ethyl acetate or DCM/methanol. In flash column chromatography, unmodified (“regular”) silica gel may be used as well as aminophase functionalized silica gel. If reference is made to flash column chromatography or to flash chromatography in the experimental section without specification of a stationary phase, regular silica gel was used.

In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### Analytical LC-MS Methods:

### Method 1

Instrument: Agilent 1290 UPLCMS 6230 TOF; column: BEH C 18 1.7 μm, 50×2.1 mm; Eluent A: water+0.05% formic acid (99%); Eluent B: acetonitrile+0.05% formic acid (99%); gradient: 0-1.7 2-90% B, 1.7-2.0 90% B; flow 1.2 ml/min; temperature: 60° C.; DAD scan: 190-400 nm.

### Method 2:

Instrument: Waters Acquity UPLCMS SingleQuad; Column: Acquity UPLC BEH C18 1.7 um, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Method 3:

Instrument: Waters Acquity UPLCMS SingleQuad; Column: Acquity UPLC BEH C18 1.7 um, 50×2.1 mm; eluent A: water+0.1 vol % formic acid (99%), eluent B: MeCN; gradient: 0-1.6 min 1-99% B, 1.6-2.0 min 99% B; flow 0.8 ml/min; temperature: 60° C.; DAD scan: 210-400 nm.

### Method 4:

System MS: Thermo Scientific FT-MS; System UHPLC+: Thermo Scientific UltiMate 3000; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm/Optimum Integration Path 210-300 nm

### Method 9:

System MS: Thermo Scientific FT-MS; System UHPLC+: Thermo Scientific Vanquish; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm Method 10:

Instrument: Knauer P2.1L, Knauer UV detector Azura UVD 2.1S, Prepcon 5 software. Column: Chromatorex C18 10 μm, 125 mm×30 mm; eluent A: water, eluent B: acetonitrile; gradient: 0-5 min 20% B; 5-18 min 20%-90% B, 18-25 min 90% B; column temperature: rt; flow rate: 50 mL/min; UV detection: 210 nm.

### Method 11:

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 ml/min; temperature: 50° C.; PDA: 220 nm & 254 nm.

### Method 12:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-0.8 min, 5-95% B, 0.8-1.2 min 95% B; flow 1.5 ml/min; temperature: 40° C.; PDA: 220 nm & 254 nm.

### Method 13:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-1.2 min, 0-60% B, 1.2-1.6 min, 60% B; flow 1.0 ml/min; temperature: 40° C.; PDA: 220 nm & 254 nm.

### Method 14:

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min 0-60% B, 0.8-1.2 min 60% B; flow 1.5 ml/min; temperature: 50° C.; PDA: 220 nm & 254 nm.

### Method 15:

Instrument: Agilent 1100\G1956A SingleQuad; Column: Kinetex@ 5 μm EVO C18 30*2.1 mm; eluent A: water+0.0375 vol % trifluoroacetic acid, eluent B: acetonitrile+0.01875 vol % trifluoroacetic acid; gradient: 0-0.8 min 0-60% B, 0.8-1.2 min 60% B; flow 1.5 ml/min; temperature: 50° C.; PDA: 220 nm & 254 nm.

### Method 16:

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 ml/min; temperature: 50° C.; PDA: 220 nm & 254 nm.

### Method 17:

Instrument: Agilent 1100\G1956A SingleQuad; Column: Kinetex@ 5 μm EVO C18 30*2.1 mm; eluent A: water+0.0375 vol % trifluoroacetic acid, eluent B: acetonitrile+0.01875 vol % trifluoroacetic acid; gradient: 0-0.8 min 5-95% B, 0.8-1.2 min 95% B; flow 1.5 ml/min; temperature: 50° C.; PDA: 220 nm & 254 nm.

### Method 18:

Instrument: Agilent 1200\G6110A SingleQuad; Column: XBridge C18 2.1*50 mm, 5 μm; eluent A: water+0.025 vol % ammonium hydroxide, eluent B: acetonitrile; gradient: 0-1.2 min 10-80% B, 1.2-1.6 min 80% B; flow 1.2 ml/min; temperature: 40° C.; DAD: 220 nm & 254 nm.

### Method 19:

Instrument: Agilent 1200\G6110A SingleQuad; Column: XBridge C18 2.1*50 mm, 5 μm; eluent A: water+0.025 vol % ammonium hydroxide, eluent B: acetonitrile; gradient: 0-1.2 min 0-60% B, 1.2-1.6 min 60% B; flow 1.0 ml/min; temperature: 40° C.; DAD: 220 nm & 254 nm.

### Method 20:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-0.8 min, 5-95% B, 0.8-1.2 min 95% B; flow 1.5 ml/min; temperature: 40° C.; PDA: 220 nm & 254 nm.

### Method 21:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-1.2 min, 0-60% B, 1.2-1.6 min, 60% B; flow 1.0 ml/min; temperature: 40° C.; PDA: 220 nm & 254 nm.

### Method 22:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-0.8 min, 5-95% B, 0.8-1.2 min, 95% B; flow 1.5 ml/min; temperature: 40° C.; PDA: 220 nm & 254 nm.

### Method 23:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-0.8 min, 0-60% B, 0.8-1.2 min, 60% B; flow 1.5 ml/min; temperature: 40° C.; PDA: 220 nm & 254 nm.

### Method 24:

Instrument: Waters Acquity UPLCMS SingleQuad; Colum: Acquity UPLC BEH C18 1.7 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm

### Preparative LC-MS Methods:

### Method 5:

Instrument: Knauer P2.1L, Knauer UV detector Azura UVD 2.1S, Prepcon 5 software. Column: Chromatorex C18 10 μm, 125 mm×30 mm; eluent A: water, eluent B: acetonitrile; gradient: 0-5 min 20% B; 5-18 min 20%-90% B, 18-25 min 90% B; column temperature: rt; flow rate: 50 mL/min; UV detection: 210 nm.

### Method 7:

Instrument: Waters Autopurification MS SingleQuad; Column: Waters XBrigde C18 5μ 100×30 mm; eluent A: water+0.2 vol. % aqueous ammonia (32%), eluent B: acetonitrile; gradient: 0-5.5 min. 5-100% B; flow 70 ml/min; temperature: 25° C.; DAD scan: 210-400 nm

### Method 8:

Instrument: Waters Autopurification MS SingleQuad; Column: Waters XBrigde C18 5μ 50×50 mm; eluent A: water+0.1 vol % formic acid, eluent B: methanol; gradient: 0-0.50 min. 20% B; flow 50 to 100 ml/min, 0.50-8.00 min. 20-60% B; flow 100 ml/min, temperature: 25° C.; DAD scan: 210-400 nm

### Method 9:

Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: given for intermediates and examples, rate 150 mL/min, temperature 25° C.; UV 220 nm

### Method 10:

Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: 0.1% ammonia in water; Eluent B: acetonitrile; gradient: given for intermediates and examples, rate 150 mL/min, temperature 25° C.; UV 220 nm

### NMR Spectra:

The multiplicities of proton signals in 1H NMR spectra given in the following paragraphs reflect the observed signal form and do not take into account any higher-order signal phenomena. As a rule, the chemical shift data refers to the center of the signal in question. In the case of wide multiplets, a range is specified. Signals hidden by solvent or water were either assigned tentatively or are not listed. Strongly broadened signals—e.g. caused by rapid rotation of molecular moieties or by interchanging protons—have also been assigned tentatively (often referred to as a broad multiplet or broad singlet) or are not shown.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

### Syntheses of Intermediate Compounds

### Intermediate 1

### tert-butyl 3-bromo-2-(4-fluorophenyl)-6,7-dihydropyrazolo[1,5-a]pyrazine-5(4H)-carboxylate

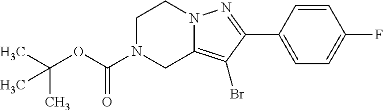 [SMILES: CC(C)(C)OC(=O)N1CCn2nc(-c3ccc(F)cc3)c(Br)c2C1]

tert-Butyl 2-(4-fluorophenyl)-6,7-dihydropyrazolo[1,5-a]pyrazine-5(4H)-carboxylate (1.00 g, 3.15 mmol; CAS-RN: [1773507-35-9]) was added to a reaction vessel along with DCM (15 ml), it was cooled to 0° C. and 1-bromopyrrolidine-2,5-dione (561 mg, 3.15 mmol; CAS-RN: [128-08-5]) was added. The mixture was stirred at rt over the weekend. Additional 1-bromopyrrolidine-2,5-dione (110 mg) was added and the mixture stirred for an additional 2 hours. Saturated NaHCO₃ solution (aqueous, 20 mL) and water (5 mL) were added, the layers separated and the aqueous layer extracted twice with DCM (total 2×20 mL), the combined organic layers were dried over Na₂SO, filtered and concentrated under reduced pressure yielding the title compound (1.4 g, 113% yield, containing some minor impurities).

LC-MS (Method 1): Rₜ=1.46 min; MS (ESIpos): m/z=396.1 [M+H]⁺

1H NMR (400 MHz, DMSO-d6) δ ppm 1.45 (s, 9H) 3.86 (m, 2H) 4.15 (m, 2H) 4.52 (s, 2H) 7.31 (m, 2H) 7.84 (m, 2H)

### Intermediate 2

### tert-butyl 2-(4-fluorophenyl)-3-(pyridin-4-yl)-6,7-dihydropyrazolo[1,5-a]pyrazine-5(4H)-carboxylate

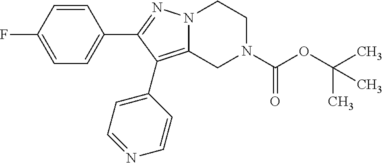 [SMILES: CC(C)(C)OC(=O)N1CCn2nc(-c3ccc(F)cc3)c(-c3ccncc3)c2C1]

tert-butyl 3-bromo-2-(4-fluorophenyl)-6,7-dihydropyrazolo[1,5-a]pyrazine-5(4H)-carboxylate (738 mg, 1.86 mmol, Intermediate 1), 4-(4,4,5,5-tetramethyl-1,3,2-dioxaborolan-2-yl)pyridine (458 mg, 2.23 mmol, CAS-RN: [181219-01-2]), Pd(dppf)Cl₂ DCM complex (126 mg, 155 μmol; CAS-RN: [95464-05-4]) and potassium carbonate (772 mg, 5.59 mmol; CAS-RN: [584-08-7]) were added to a microwave reaction vessel, the reaction vessel flushed with nitrogen and sealed. Degassed THF (20 ml) and water (2.5 ml) were added and the mixture heated at 110° C. for 1 hour in a microwave. ethyl acetate (100 ml) was added, and the organic phase was then washed with NaHCO₃ solution (aqueous, saturated), dried over sodium sulfate, and concentrated under reduced pressure. The residue was dissolved residue in ca 5 ml ethyl acetate and filtered through a 2 g silica column, and washed with additional ethyl acetate (5 ml), and the combined fractions concentrated under reduced pressure yielding the title compound (800 mg, 90% purity, 98% yield).

LC-MS (Method 3): Rₜ=0.95 min; MS (ESIpos): m/z=396.1 [M+H]⁺

¹H NMR (400 MHz, DMSO-d6) δ ppm 1.41 (s, 9H) 3.89 (m, 2H) 4.21 (m, 2H) 4.66 (s, 2H) 7.14 (d, J=5.00 Hz, 2H) 7.19 (m, J=8.19 Hz, 2H) 7.37 (m, 2H) 8.53 (m, 2H)

### Intermediate 3

### 2-(4-fluorophenyl)-3-(pyridin-4-yl)-4,5,6,7-tetrahydropyrazolo[1,5-a]pyrazine

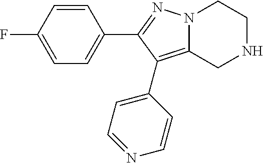 [SMILES: Fc1ccc(-c2nn3c(c2-c2ccncc2)CNCC3)cc1]

tert-Butyl 2-(4-fluorophenyl)-3-(pyridin-4-yl)-6,7-dihydropyrazolo[1,5-a]pyrazine-5(4H)-carboxylate (512 mg, 1.30 mmol, Intermediate 2) was added to a reaction vessel, followed by DCM (3.8 ml) and then the dropwise addition of trifluoroacetic acid (1.5 ml, 19 mmol; CAS-RN: [76-05-1]). The mixture was stirred at rt for 1.5 hours, under nitrogen. The mixture was concentrated under reduced pressure. DCM (50 ml) was added and the mixture stirred with NaHCO₃(saturated, aqueous, 30 ml), the layers separated, the aqueous layer was extracted with DCM (30 ml), the combined layers dried over sodium sulfate and concentrated under reduced pressure. Further purification by silica gel column chromatography (Biotage Sfär Silica HC D, 25 g), Eluent: DCM/Ethanol from 100/0 to 0/100 over 20 min, with a flowrate of 60 mL/min fractions yielded the title compound (372 mg, 97% yield).

LC-MS (Method 3): Rₜ=0.50 min; MS (ESIpos): m/z=295.1 [M+H]⁺

¹H NMR (400 MHz, DMSO-d6) δ ppm 3.22-3.32 (m, 2H) 4.09 (s, 2H) 4.15 (m, 2H) 7.11 (dd, 2H) 7.14-7.24 (m, 2H) 7.37 (m, 2H) 8.50 (dd, 2H)

### Intermediate 4

### trifluoroacetic acid-2-(4-fluorophenyl)-3-(pyridin-4-yl)-4,5,6,7-tetrahydropyrazolo[1,5-a]pyrazine (1/1)

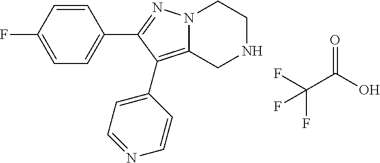

tert-butyl 2-(4-fluorophenyl)-3-(pyridin-4-yl)-6,7-dihydropyrazolo[1,5-a]pyrazine-5(4H)-carboxylate (550 mg, 1.39 mmol) was added to a reaction vessel, followed by DCM (11 ml) and then the dropwise addition of trifluoroacetic acid (1.1 ml, 14 mmol; CAS-RN: [76-05-1]). The mixture was stirred at 40° C. for 4 hours. The reaction mixture was concentrated under reduced pressure to yield the title compound which was used without further purification in the next reaction.

LC-MS (Method 1): Rₜ=0.32 min; MS (ESIpos): m/z=295.1 [M+H]⁺

1H-NMR (400 MHz, DMSO-d6) delta [ppm]: 0.068 (0.73), 1.066 (13.41), 1.107 (1.61), 1.156 (3.07), 1.232 (0.68), 1.256 (0.48), 1.427 (0.43), 1.532 (16.00), 2.518 (8.70), 2.523 (5.60), 2.562 (0.65), 2.728 (1.11), 2.888 (1.36), 3.739 (1.86), 3.754 (3.24), 3.769 (1.91), 4.337 (0.93), 4.352 (1.29), 4.366 (0.91), 4.442 (2.56), 4.457 (4.16), 4.471 (3.61), 4.610 (7.08), 4.964 (1.02), 5.758 (1.08), 6.692 (1.76), 7.224 (3.75), 7.246 (8.07), 7.268 (4.52), 7.400 (3.61), 7.405 (1.74), 7.414 (4.08), 7.423 (3.66), 7.436 (6.40), 7.450 (3.95), 7.537 (0.43), 7.547 (1.03), 7.565 (1.16), 7.573 (1.01), 7.595 (1.38), 7.612 (1.16), 7.622 (1.41), 7.641 (0.83), 7.803 (0.86), 7.816 (0.95), 7.825 (0.93), 7.839 (0.82), 8.711 (5.05), 8.727 (4.77), 9.613 (0.51).

Intermediate 5

### tert-butyl 2-(4-chloro-3-methylphenyl)-6,7-dihydropyrazolo[1,5-a]pyrazine-5(4H)-carboxylate

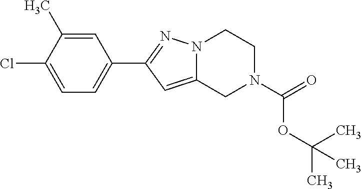 [SMILES: Cc1cc(-c2cc3n(n2)CCN(C(=O)OC(C)(C)C)C3)ccc1Cl]

tert-butyl 2-bromo-6,7-dihydropyrazolo[1,5-a]pyrazine-5(4H)-carboxylate (160 mg, 529 μmol, CAS-RN: [1250998-21-0]) and 2-(4-chloro-3-methylphenyl)-4,4,5,5-tetramethyl-1,3,2-dioxaborolane (174 mg, 688 μmol) were added to DMF (4.2 ml) and then a solution of potassium carbonate (790 μl, 2.0 M, 1.6 mmol; CAS-RN: [584-08-7]) was added in a microwave reaction vessel. The reaction vessel was flushed with nitrogen and tetrakis(triphenylphosphin) palladium (0) (61.2 mg, 52.9 μmol; CAS-RN: [14221-01-3]) was added. The sealed vessel was heated at 110° C. for 30 min in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-20%) yielding the title compound 126 mg (93% purity, 64% yield).

LC-MS (Method 1): Rₜ=1.4 min; MS (ESIpos): m/z=348.1 [M+H]⁺

¹H-NMR (400 MHz, DMSO-d6) δ [ppm]: 1.441 (16.00), 2.359 (3.58), 3.834 (0.64), 3.848 (0.44), 4.117 (0.52), 4.132 (0.76), 4.145 (0.43), 4.623 (0.81), 6.596 (1.32), 7.402 (0.79), 7.423 (0.97), 7.753 (0.62), 7.757 (0.58).

### Intermediate 6

### tert-butyl 3-bromo-2-(4-chloro-3-methylphenyl)-6,7-dihydropyrazolo[1,5-a]pyrazine-5(4H)-carboxylate

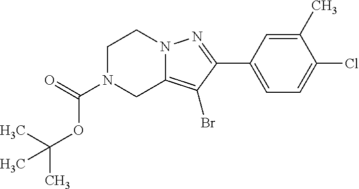 [SMILES: Cc1cc(-c2nn3c(c2Br)CN(C(=O)OC(C)(C)C)CC3)ccc1Cl]

tert-butyl 2-(4-chloro-3-methylphenyl)-6,7-dihydropyrazolo[1,5-a]pyrazine-5(4H)-carboxylate (125 mg, 359 μmol, Intermediate 5) was added to a reaction vessel along with DCM (5 ml), it was cooled to 0° C. and 1-bromopyrrolidine-2,5-dione (67.2 mg, 377 μmol) was added. The mixture was stirred at Rₜ for 1 hour. Saturated NaHCO₃ solution and water were added, the layers separated and the aqueous layer extracted twice with DCM, the combined organic layers were dried over a water-repellent filter and concentrated under reduced pressure yielding the title compound (176 mg, 100% yield, 87% purity).

LC-MS (Method 1): Rₜ=1.6 min; MS (ESIpos): m/z=427.9 [M+H]⁺

¹H-NMR (400 MHz, DMSO-d6) δ [ppm]: 1.451 (16.00), 1.461 (1.79), 2.382 (4.33), 2.518 (1.41), 2.523 (0.97), 2.563 (2.94), 3.856 (0.69), 3.870 (0.46), 4.144 (0.57), 4.158 (0.85), 4.171 (0.48), 4.519 (1.42), 5.759 (4.36), 7.500 (0.87), 7.520 (1.09), 7.657 (0.52), 7.663 (0.53), 7.683 (0.41), 7.780 (0.78), 7.784 (0.70).

### Intermediate 7

### tert-butyl 2-(4-chloro-3-methylphenyl)-3-(pyridin-4-yl)-6,7-dihydropyrazolo[1,5-a]pyrazine-5(4H)-carboxylate

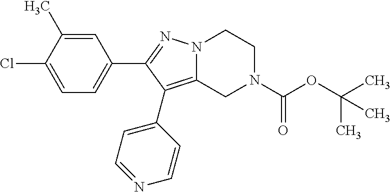 [SMILES: Cc1cc(-c2nn3c(c2-c2ccncc2)CN(C(=O)OC(C)(C)C)CC3)ccc1Cl]

tert-butyl 3-bromo-2-(4-chloro-3-methylphenyl)-6,7-dihydropyrazolo[1,5-a]pyrazine-5(4H)-carboxylate (170 mg, 398 μmol, Intermediate 6) were added to a mixture of THF (4.2 ml) and water (530 μl) in a microwave reaction vessel. The reaction vessel flushed with nitrogen and 4-(4,4,5,5-tetramethyl-1,3,2-dioxaborolan-2-yl)pyridine (114 mg, 558 μmol), potassium carbonate (165 mg, 1.20 mmol; CAS-RN: [584-08-7]) and dichloro[1,1′-bis(dialkyl/diarylphosphino) ferrocene]palladium (II) (9.76 mg, 12.0 μmol; CAS-RN: [95464-05-4]) were added. The sealed vessel was heated at 110° C. for 1 hour in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-50%) yielding the title compound 102 mg (90% purity, 54% yield).

LC-MS (Method 1): Rₜ=1.0 min; MS (ESIpos): m/z=425.1 [M+H]⁺

¹H-NMR (400 MHz, DMSO-d6) δ [ppm]: 1.066 (0.84), 1.084 (0.53), 1.154 (2.45), 1.172 (5.31), 1.190 (2.63), 1.259 (0.72), 1.414 (15.06), 1.463 (0.84), 1.987 (8.81), 2.291 (16.00), 2.322 (1.44), 2.326 (1.05), 2.332 (0.72), 2.518 (4.92), 2.522 (3.39), 2.562 (0.49), 2.664 (0.68), 2.668 (0.90), 2.673 (0.67), 3.878 (1.47), 3.891 (2.64), 3.905 (1.78), 3.999 (0.59), 4.017 (1.74), 4.035 (1.70), 4.053 (0.57), 4.199 (1.97), 4.213 (3.09), 4.226 (1.75), 4.662 (5.12), 7.062 (1.01), 7.082 (1.15), 7.149 (6.68), 7.153 (3.90), 7.160 (3.97), 7.164 (6.82), 7.181 (0.42), 7.354 (3.90), 7.374 (3.37), 7.416 (2.76), 7.420 (2.68), 8.536 (6.51), 8.540 (3.84), 8.547 (3.92), 8.551 (6.40), 8.559 (0.44), 8.563 (0.51).

### Intermediate 8

### trifluoroacetic acid-2-(4-chloro-3-methylphenyl)-3-(pyridin-4-yl)-4,5,6,7-tetrahydropyrazolo[1,5-a]pyrazine (1/1)

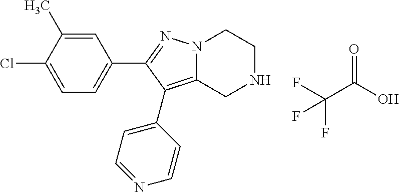

tert-butyl 2-(4-chloro-3-methylphenyl)-3-(pyridin-4-yl)-6,7-dihydropyrazolo[1,5-a]pyrazine-5(4H)-carboxylate (100 mg, 235 μmol, Intermediate 7) was added to a reaction vessel, followed by DCM (1.8 ml) and then the dropwise addition of trifluoroacetic acid (180 μl, 2.4 mmol; CAS-RN: [76-05-1]). The mixture was stirred at Rₜ for 2 hours. After the addition of further 150 μl TFA and stirring for an additional 1 hour the mixture was concentrated under reduced pressure to yield the title compound which was used without further purification in the next reaction.

LC-MS (Method 2): Rₜ=1.01 min; MS (ESIpos): m/z=325.2 [M+H]⁺

### Intermediate 9

### tert-butyl 2-(4-chlorophenyl)-6,7-dihydropyrazolo[1,5-a]pyrazine-5(4H)-carboxylate

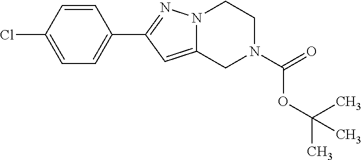 [SMILES: CC(C)(C)OC(=O)N1CCn2nc(-c3ccc(Cl)cc3)cc2C1]

tert-butyl 2-bromo-6,7-dihydropyrazolo[1,5-a]pyrazine-5(4H)-carboxylate (160 mg, 529 μmol, CAS-RN: [1250998-21-0]) and 2-(4-chlorophenyl)-4,4,5,5-tetramethyl-1,3,2-dioxaborolane (164 mg, 688 μmol) were added to DMF (4.2 ml) and then a solution of potassium carbonate (790 μl, 2.0 M, 1.6 mmol; CAS-RN: [584-08-7]) was added in a microwave reaction vessel. The reaction vessel was flushed with nitrogen and tetrakis(triphenylphosphin) palladium (0): (61.2 mg, 52.9 μmol; CAS-RN: [14221-01-3]) was added. The sealed vessel was heated at 110° C. for 30 min in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-20%) yielding the title compound 132 mg (90% purity, 67% yield).

LC-MS (Method 1): Rₜ=1.3 min; MS (ESIpos): m/z=334.1 [M+H]⁺

¹H-NMR (400 MHz, DMSO-d6) δ [ppm]: 1.441 (16.00), 1.448 (0.99), 2.518 (0.58), 2.523 (0.41), 3.833 (0.64), 3.847 (0.45), 4.123 (0.53), 4.137 (0.75), 4.151 (0.43), 4.625 (0.77), 6.615 (1.40), 7.436 (1.42), 7.440 (0.43), 7.452 (0.50), 7.457 (1.71), 7.764 (1.73), 7.769 (0.51), 7.781 (0.47), 7.786 (1.42), 8.173 (0.53).

### Intermediate 10

### tert-butyl 3-bromo-2-(4-chlorophenyl)-6,7-dihydropyrazolo[1,5-a]pyrazine-5(4H)-carboxylate

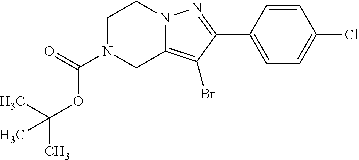 [SMILES: CC(C)(C)OC(=O)N1CCn2nc(-c3ccc(Cl)cc3)c(Br)c2C1]

tert-butyl 2-(4-chlorophenyl)-6,7-dihydropyrazolo[1,5-a]pyrazine-5(4H)-carboxylate (130 mg, 389 μmol, Intermediate 9) was added to a reaction vessel along with DCM (2 ml), it was cooled to 0° C. and 1-bromopyrrolidine-2,5-dione (72.8 mg, 409 μmol) was added. The mixture was stirred at rt for 1 hour. Saturated NaHCO₃ solution and water were added, the layers separated and the aqueous layer extracted twice with DCM, the combined organic layers were dried over a water-repellent filter and concentrated under reduced pressure yielding the title compound (177 mg, 99% yield, 90% purity).

LC-MS (Method 1): Rₜ=1.5 min; MS (ESIpos): m/z=413.9 [M+H]⁺

¹H-NMR (400 MHz, DMSO-d6) δ [ppm]: 1.451 (16.00), 2.518 (0.50), 2.563 (2.94), 3.843 (0.40), 3.856 (0.70), 3.870 (0.48), 4.149 (0.59), 4.163 (0.84), 4.176 (0.49), 4.522 (1.43), 5.759 (3.29), 7.528 (1.78), 7.533 (0.57), 7.545 (0.66), 7.550 (2.19), 7.827 (2.18), 7.832 (0.63), 7.844 (0.58), 7.849 (1.68).

### Intermediate 11

### tert-butyl 2-(4-chlorophenyl)-3-(pyridin-4-yl)-6,7-dihydropyrazolo[1,5-a]pyrazine-5(4H)-carboxylate

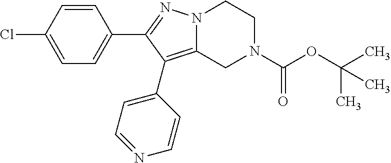 [SMILES: CC(C)(C)OC(=O)N1CCn2nc(-c3ccc(Cl)cc3)c(-c3ccncc3)c2C1]

tert-butyl 3-bromo-2-(4-chlorophenyl)-6,7-dihydropyrazolo[1,5-a]pyrazine-5(4H)-carboxylate (170 mg, 412 μmol, Intermediate 10) were added to a mixture of THF (4.3 ml) and water (500 μl) in a microwave reaction vessel. The reaction vessel flushed with nitrogen and 4-(4,4,5,5-tetramethyl-1,3,2-dioxaborolan-2-yl)pyridine (118 mg, 577 μmol), potassium carbonate (171 mg, 1.24 mmol; CAS-RN: [584-08-7]) and dichloro[1,1′-bis(dialkyl/diarylphosphino) ferrocene]palladium (II) (27.9 mg, 34.2 μmol; CAS-RN: [95464-05-4]) were added. The sealed vessel was heated at 110° C. for 1 hour in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-80%) yielding the title compound 96.4 mg (90% purity, 51% yield).

LC-MS (Method 1): Rₜ=0.9 min; MS (ESIpos): m/z=411.1 [M+H]⁺

¹H-NMR (400 MHz, DMSO-d6) δ [ppm]: 1.066 (16.00), 1.414 (3.36), 3.892 (0.60), 3.905 (0.40), 3.940 (2.85), 4.205 (0.45), 4.219 (0.69), 4.662 (1.16), 7.151 (1.48), 7.155 (0.91), 7.162 (0.92), 7.167 (1.48), 7.343 (0.72), 7.364 (1.29), 7.412 (2.03), 7.417 (0.51), 7.433 (1.08), 8.541 (1.60), 8.545 (0.88), 8.552 (0.89), 8.557 (1.50).

### Intermediate 12

### trifluoroacetic acid-2-(4-chlorophenyl)-3-(pyridin-4-yl)-4,5,6,7-tetrahydropyrazolo[1,5-a]pyrazine (1/1)

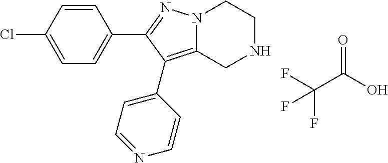

tert-butyl 2-(4-chlorophenyl)-3-(pyridin-4-yl)-6,7-dihydropyrazolo[1,5-a]pyrazine-5(4H)-carboxylate (93.0 mg, 226 μmol, Intermediate 11) was added to a reaction vessel, followed by DCM (1.7 ml) and then the dropwise addition of trifluoroacetic acid (170 μl, 2.3 mmol; CAS-RN: [76-05-1]). The mixture was stirred at rt for 2 hours. After the addition of further 150 μl TFA and stirring for an additional 1 hour the mixture was concentrated under reduced pressure to yield the title compound which was used without further purification in the next reaction.

LC-MS (Method 2): Rₜ=0.94 min; MS (ESIpos): m/z=311.2 [M+H]⁺

### Intermediate 13

### tert-butyl 2-(3-fluorophenyl)-6,7-dihydropyrazolo[1,5-a]pyrazine-5(4H)-carboxylate

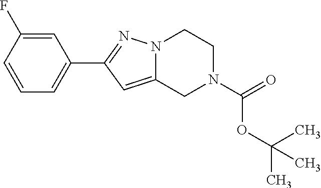 [SMILES: CC(C)(C)OC(=O)N1CCn2nc(-c3cccc(F)c3)cc2C1]

tert-butyl 2-bromo-6,7-dihydropyrazolo[1,5-a]pyrazine-5(4H)-carboxylate (200 mg, 662 μmol, CAS-RN: [1250998-21-0]) and 2-(3-fluorophenyl)-4,4,5,5-tetramethyl-1,3,2-dioxaborolane (191 mg, 860 μmol) were added to DMF (8.4 ml) and then a solution of potassium carbonate (990 μl, 2.0 M, 2.0 mmol; CAS-RN: [584-08-7]) was added in a microwave reaction vessel. The reaction vessel was flushed with nitrogen and tetrakis(triphenylphosphin) palladium (0) (76.5 mg, 66.2 μmol; CAS-RN: [14221-01-3]) was added. The sealed vessel was heated at 110° C. for 30 min in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-16%) yielding the title compound 195 mg (95% purity, 88% yield).

LC-MS (Method 1): Rₜ=1.3 min; MS (ESIpos): m/z=318.1 [M+H]⁺

¹H-NMR (400 MHz, DMSO-d6) δ [ppm]: 1.066 (0.67), 1.430 (0.45), 1.442 (16.00), 2.518 (0.41), 3.837 (0.66), 3.852 (0.45), 4.133 (0.56), 4.147 (0.79), 4.160 (0.46), 4.629 (0.82), 6.656 (1.38), 7.426 (0.41), 7.442 (0.41), 7.595 (0.40), 7.597 (0.53), 7.618 (0.41).

### Intermediate 14

### tert-butyl 3-bromo-2-(3-fluorophenyl)-6,7-dihydropyrazolo[1,5-a]pyrazine-5(4H)-carboxylate

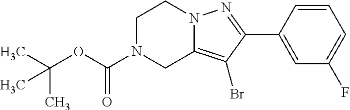 [SMILES: CC(C)(C)OC(=O)N1CCn2nc(-c3cccc(F)c3)c(Br)c2C1]

tert-butyl 2-(3-fluorophenyl)-6,7-dihydropyrazolo[1,5-a]pyrazine-5(4H)-carboxylate (190 mg, 599 μmol, Intermediate 13) was added to a reaction vessel along with DCM (2 ml), it was cooled to 0° C. and 1-bromopyrrolidine-2,5-dione (72.8 mg, 409 μmol) was added. The mixture was stirred at rt for 2 hour. Saturated NaHCO₃ solution and water were added, the layers separated and the aqueous layer extracted twice with DCM, the combined organic layers were dried over a water-repellent filter and concentrated under reduced pressure yielding the title compound (306 mg, 99% yield, 77% purity).

LC-MS (Method 1): Rₜ=1.4 min; MS (ESIpos): m/z=396 [M+H]⁺

¹H-NMR (400 MHz, DMSO-d6) δ [ppm]: 1.065 (1.22), 1.154 (0.66), 1.172 (1.37), 1.190 (0.70), 1.443 (1.59), 1.453 (16.00), 1.987 (2.42), 2.518 (0.60), 2.523 (0.42), 2.563 (11.27), 3.847 (0.44), 3.861 (0.72), 3.874 (0.49), 4.017 (0.55), 4.035 (0.55), 4.158 (0.64), 4.172 (0.89), 4.186 (0.51), 4.528 (1.46), 7.517 (0.50), 7.533 (0.50), 7.554 (0.47), 7.677 (0.52), 7.679 (0.65), 7.683 (0.48), 7.697 (0.41), 7.700 (0.51).

### Intermediate 15

### tert-butyl 2-(3-fluorophenyl)-3-(pyridin-4-yl)-6,7-dihydropyrazolo[1,5-a]pyrazine-5(4H)-carboxylate

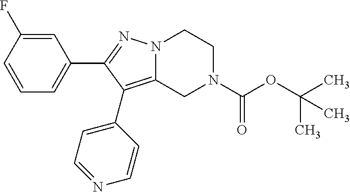 [SMILES: CC(C)(C)OC(=O)N1CCn2nc(-c3cccc(F)c3)c(-c3ccncc3)c2C1]

tert-butyl 3-bromo-2-(3-fluorophenyl)-6,7-dihydropyrazolo[1,5-a]pyrazine-5(4H)-carboxylate (250 mg, 77% purity, 486 μmol, Intermediate 14) were added to a mixture of THF (4.5 ml) and water (450 μl) in a microwave reaction vessel. The reaction vessel flushed with nitrogen and 4-(4,4,5,5-tetramethyl-1,3,2-dioxaborolan-2-yl)pyridine (130 mg, 632 μmol), potassium carbonate (201 mg, 1.46 mmol; CAS-RN: [584-08-7]) and dichloro[1,1′-bis(dialkyl/diarylphosphino)ferrocene]palladium (II) (32.9 mg, 40.3 μmol; CAS-RN: [95464-05-4]) were added. The sealed vessel was heated at 110° C. for 1 hour in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-90%) yielding the title compound 176 mg (85% purity, 78% yield).

LC-MS (Method 1): Rₜ=0.9 min; MS (ESIpos): m/z=395.1 [M+H]⁺

¹H-NMR (400 MHz, DMSO-d6) δ [ppm]: 1.066 (16.00), 1.154 (0.63), 1.172 (1.33), 1.190 (0.67), 1.416 (1.21), 1.987 (2.32), 3.938 (2.35), 4.017 (0.46), 4.034 (0.44), 4.658 (0.42), 7.168 (0.52), 7.183 (0.42), 8.551 (0.44), 8.567 (0.41).

### Intermediate 16

### trifluoroacetic acid-2-(3-fluorophenyl)-3-(pyridin-4-yl)-4,5,6,7-tetrahydropyrazolo[1,5-a]pyrazine (1/1)

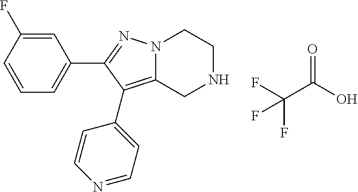

tert-butyl 2-(3-fluorophenyl)-3-(pyridin-4-yl)-6,7-dihydropyrazolo[1,5-a]pyrazine-5(4H)-carboxylate (175 mg, 444 μmol, Intermediate 15) was added to a reaction vessel, followed by DCM (3.4 ml) and then the dropwise addition of trifluoroacetic acid (340 μl, 4.4 mmol; CAS-RN: [76-05-1]). The mixture was stirred at rt for 3 hours. The solution was concentrated under reduced pressure to yield the title compound which was used without further purification in the next reaction.

LC-MS (Method 2): Rₜ=0.85 min; MS (ESIpos): m/z=295.3 [M+H]⁺

### Intermediate 17

### tert-butyl 2-(3-methoxyphenyl)-6,7-dihydropyrazolo[1,5-a]pyrazine-5(4H)-carboxylate

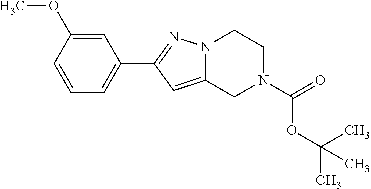 [SMILES: COc1cccc(-c2cc3n(n2)CCN(C(=O)OC(C)(C)C)C3)c1]

tert-butyl 2-bromo-6,7-dihydropyrazolo[1,5-a]pyrazine-5(4H)-carboxylate (250 mg, 827 μmol; CAS-RN: [1250998-21-0]) and 2-(3-methoxyphenyl)-4,4,5,5-tetramethyl-1,3,2-dioxaborolane (252 mg, 1.08 mmol) were added to DMF (10 ml) and then a solution of potassium carbonate (1.2 ml, 2.0 M, 2.5 mmol; CAS-RN: [584-08-7]) was added in a microwave reaction vessel. The reaction vessel was flushed with nitrogen and tetrakis(triphenylphosphin) palladium (0) (95.6 mg, 82.7 μmol; CAS-RN: [14221-01-3]) was added. The sealed vessel was heated at 110° C. for 30 min in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-25%) yielding the title compound 220 mg (90% purity, 73% yield).

LC-MS (Method 1): Rₜ=1.2 min; MS (ESIpos): m/z=330.1 [M+H]⁺

¹H-NMR (400 MHz, DMSO-d6) δ [ppm]: 1.066 (16.00), 1.156 (2.29), 1.426 (2.20), 1.430 (0.92), 1.442 (12.90), 2.518 (0.54), 2.729 (1.37), 2.888 (1.54), 3.677 (0.67), 3.784 (5.90), 3.833 (0.53), 3.938 (3.00), 4.123 (0.43), 4.138 (0.62), 4.623 (0.61), 6.593 (1.12), 7.295 (1.05), 7.301 (0.47), 7.303 (0.45), 7.314 (0.62), 7.317 (0.59), 7.321 (0.75), 7.324 (0.42).

### Intermediate 18

### tert-butyl 3-bromo-2-(3-methoxyphenyl)-6,7-dihydropyrazolo[1,5-a]pyrazine-5(4H)-carboxylate

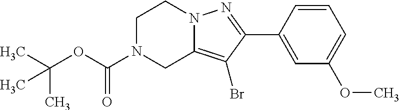 [SMILES: COc1cccc(-c2nn3c(c2Br)CN(C(=O)OC(C)(C)C)CC3)c1]

tert-butyl 2-(3-methoxyphenyl)-6,7-dihydropyrazolo[1,5-a]pyrazine-5(4H)-carboxylate (213 mg, 90% purity, 582 μmol, Intermediate 17) was added to a reaction vessel along with DCM (2 ml), it was cooled to 0° C. and 1-bromopyrrolidine-2,5-dione (104 mg, 582 μmol) was added. The mixture was stirred at rt for 2 hour. Saturated NaHCO₃ solution and water were added, the layers separated and the aqueous layer extracted twice with DCM, the combined organic layers were dried over a water-repellent filter and concentrated under reduced pressure yielding the title compound 290 mg (59% purity, 72% yield).

LC-MS (Method 1): Rₜ=1.4 min; MS (ESIpos): m/z=410 [M+H]⁺

¹H-NMR (400 MHz, DMSO-d6) δ [ppm]: 1.066 (7.46), 1.154 (0.75), 1.172 (1.39), 1.190 (0.71), 1.425 (3.34), 1.429 (1.32), 1.443 (3.22), 1.453 (16.00), 1.987 (2.51), 2.518 (0.40), 2.563 (9.14), 3.765 (0.89), 3.784 (1.53), 3.792 (7.79), 3.843 (0.50), 3.856 (0.78), 3.870 (0.51), 3.939 (1.14), 4.017 (0.57), 4.035 (0.58), 4.148 (0.68), 4.162 (0.92), 4.174 (0.51), 4.521 (1.44), 6.959 (0.44), 6.972 (0.41), 6.977 (0.43), 6.978 (0.48), 6.983 (0.40), 7.336 (0.69), 7.339 (0.81), 7.343 (0.67), 7.344 (0.64), 7.375 (0.97), 7.393 (1.45), 7.397 (1.19), 7.400 (0.71).

### Intermediate 19

### tert-butyl 2-(3-methoxyphenyl)-3-(pyridin-4-yl)-6,7-dihydropyrazolo[1,5-a]pyrazine-5(4H)-carboxylate

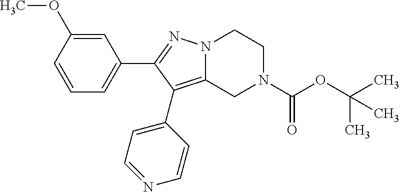 [SMILES: COc1cccc(-c2nn3c(c2-c2ccncc2)CN(C(=O)OC(C)(C)C)CC3)c1]

tert-butyl 3-bromo-2-(3-methoxyphenyl)-6,7-dihydropyrazolo[1,5-a]pyrazine-5(4H)-carboxylate (210 mg, 514 μmol, Intermediate 18) were added to a mixture of THF (5.4 ml) and water (540 μl) in a microwave reaction vessel. The reaction vessel flushed with nitrogen and 4-(4,4,5,5-tetramethyl-1,3,2-dioxaborolan-2-yl)pyridine (137 mg, 669 μmol), potassium carbonate (213 mg, 1.54 mmol; CAS-RN: [584-08-7]) and dichloro[1,1′-bis(dialkyl/diarylphosphino) ferrocene]palladium (II) (34.9 mg, 42.7 μmol; CAS-RN: [95464-05-4]) were added. The sealed vessel was heated at 110° C. for 1 hour in a microwave. ethyl acetate and water was added. After extraction 2× with ethyl acetate the combined organic phases were dried with an water-repellent filter and concentrated under reduced pressure. The residue was purified by silica gel column chromatography (eluent: ethyl acetate/hexane 0-90%) yielding the title compound 129 mg (85% purity, 52% yield).

LC-MS (Method 1): Rₜ=0.8 min; MS (ESIpos): m/z=407.1 [M+H]⁺

¹H-NMR (400 MHz, DMSO-d6) δ [ppm]: 1.154 (4.11), 1.172 (9.37), 1.190 (4.86), 1.416 (4.35), 1.435 (0.94), 1.987 (16.00), 2.518 (0.72), 2.523 (0.50), 3.667 (8.47), 3.878 (0.45), 3.891 (0.79), 3.905 (0.51), 3.999 (1.06), 4.017 (3.13), 4.035 (3.20), 4.053 (1.05), 4.203 (0.60), 4.217 (0.92), 4.230 (0.51), 4.655 (1.51), 5.758 (7.65), 6.875 (0.54), 6.879 (0.58), 6.882 (0.40), 6.886 (1.04), 6.889 (1.25), 6.892 (1.09), 6.896 (1.58), 6.902 (1.04), 6.906 (0.56), 7.155 (1.87), 7.159 (1.04), 7.166 (1.05), 7.170 (1.88), 7.226 (0.53), 7.246 (0.62), 7.250 (0.48), 7.268 (0.43), 7.544 (0.43), 7.547 (0.57), 7.549 (0.55), 7.555 (0.48), 7.558 (0.41), 7.565 (0.68), 7.566 (0.52), 7.572 (0.57), 7.574 (0.47), 7.591 (0.44), 7.596 (0.83), 7.613 (0.70), 7.615 (0.62), 7.622 (0.75), 7.625 (0.87), 7.630 (0.44), 7.642 (0.47), 7.645 (0.45), 8.531 (1.92), 8.535 (1.04), 8.542 (1.06), 8.546 (1.83).

### Intermediate 20

### trifluoroacetic acid-2-(3-methoxyphenyl)-3-(pyridin-4-yl)-4,5,6,7-tetrahydropyrazolo[1,5-a]pyrazine (1/1)

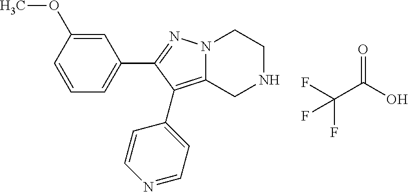

tert-butyl 2-(3-methoxyphenyl)-3-(pyridin-4-yl)-6,7-dihydropyrazolo[1,5-a]pyrazine-5(4H)-carboxylate (130 mg, 320 μmol, Intermediate 19) was added to a reaction vessel, followed by DCM (2.5 ml) and then the dropwise addition of trifluoroacetic acid (250 μl, 3.2 mmol; CAS-RN: [76-05-1]). The mixture was stirred at rt for 3 hours. The solution was concentrated under reduced pressure to yield the title compound which was used without further purification in the next reaction.

LC-MS (Method 2): Rₜ=0.82 min; MS (ESIpos): m/z=307.2 [M+H]⁺

### Intermediate 21

### tert-butyl 3-{2-[(cyclopropanecarbonyl)amino]pyridin-4-yl}-2-(4-fluorophenyl)-6,7-dihydropyrazolo[1,5-a]pyrazine-5(4H)-carboxylate

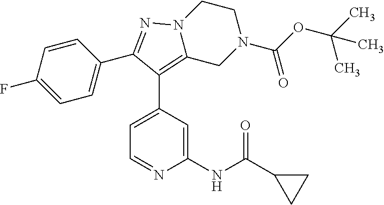 [SMILES: CC(C)(C)OC(=O)N1CCn2nc(-c3ccc(F)cc3)c(-c3ccnc(NC(=O)C4CC4)c3)c2C1]

tert-Butyl 3-bromo-2-(4-fluorophenyl)-6,7-dihydropyrazolo[1,5-a]pyrazine-5(4H)-carboxylate (100 mg, 252 μmol, Intermediate 1), N-[4-(4,4,5,5-tetramethyl-1,3,2-dioxaborolan-2-yl)pyridin-2-yl]cyclopropanecarboxamide (80.0 mg, 278 μmol, CAS-RN: [1286230-87-2]), THF (2.7 ml), potassium carbonate (105 mg, 757 μmol; CAS-RN: [584-08-7]) and water (330 μl) were added to a microwave vessel. After degassing the mixture, Pd(dppf)Cl₂ DCM complex (17.1 mg, 20.9 μmol; CAS-RN: [95464-05-4]) was added and the vessel was sealed under nitrogen. The mixture was stirred at 110° C. for 1 h in a microwave. ethyl acetate (25 ml) was added, and the mixture was washed with water, NaHCO₃ solution and saturated sodium chloride solution (each 20 mL), the organic phase was dried over sodium sulfate, filtered and concentrated under reduced pressure yielding the crude title compound (118 mg).

LC-MS (Method 3): Rₜ=1.23 min; MS (ESIpos): m/z=478.3 [M+H]⁺

### Intermediate 22

### N-{4-[2-(4-fluorophenyl)-4,5,6,7-tetrahydropyrazolo[1,5-a]pyrazin-3-yl]pyridin-2-yl}cyclopropanecarboxamide

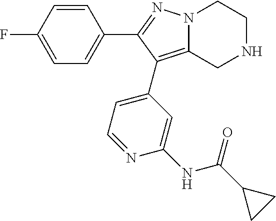 [SMILES: O=C(Nc1cc(-c2c(-c3ccc(F)cc3)nn3c2CNCC3)ccn1)C1CC1]

To a solution of tert-butyl 3-{2-[(cyclopropanecarbonyl)amino]pyridin-4-yl}-2-(4-fluorophenyl)-6,7-dihydropyrazolo[1,5-a]pyrazine-5(4H)-carboxylate (118 mg, 247 μmol, Intermediate 21) in DCM (2.1 ml) was added TFA (620 μl, 4.0 M, 2.5 mmol; CAS-RN: [76-05-1]) under nitrogen. The mixture was stirred at rt for 3 h. The precipitate was filtered yielding the crude title compound as the TFA salt. (93.3 mg). The free base can be obtained by basic aqueous workup with extraction into ethyl acetate.

LC-MS (Method 3): Rₜ=0.70 min; MS (ESIpos): m/z=378.2 [M+H]⁺

### Intermediate 23

### tert-butyl 3-{2-[(tert-butoxycarbonyl)amino]pyridin-4-yl}-2-(4-fluorophenyl)-6,7-dihydropyrazolo[1,5-a]pyrazine-5(4H)-carboxylate

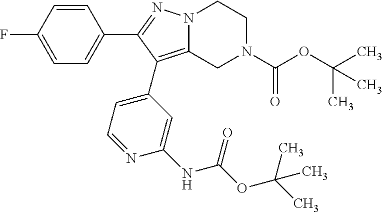 [SMILES: CC(C)(C)OC(=O)Nc1cc(-c2c(-c3ccc(F)cc3)nn3c2CN(C(=O)OC(C)(C)C)CC3)ccn1]

tert-butyl 3-bromo-2-(4-fluorophenyl)-6,7-dihydropyrazolo[1,5-a]pyrazine-5(4H)-carboxylate (110 mg, 278 μmol, Intermediate 1), tert-butyl [4-(4,4,5,5-tetramethyl-1,3,2-dioxaborolan-2-yl)pyridin-2-yl]carbamate (107 mg, 333 μmol, CAS-RN: [1095708-32-9]), (18.8 mg, 23.0 μmol; CAS-RN: [95464-05-4]) and Pd(dppf)Cl₂ DCM complex (115 mg, 833 μmol; CAS-RN: [584-08-7]) were added to a microwave reaction vessel, the vessel was flushed with nitrogen and sealed. Degassed THF (2.9 ml) and water (370 μl) were added and the mixture heated at 110° C. for 1 hour in a microwave. ethyl acetate (100 ml) was added, and the mixture washed with NaHCO₃ solution (aqueous, saturated), the organic phase dried over sodium sulfate, filtered, and concentrated under reduced pressure. The residue was dissolved in ethyl acetate (ca 5 ml) and filtered through a 2 g silica column, and the column washed with an additional ethyl acetate (5 ml). The combined organic phases were concentrated under reduced pressure yielding the crude title compound (140 mg).

LC-MS (Method 3): Rₜ=1.41 min; MS (ESIpos): m/z=510 [M+H]⁺

### Intermediate 24

### 4-[2-(4-fluorophenyl)-4,5,6,7-tetrahydropyrazolo[1,5-a]pyrazin-3-yl]pyridin-2-amine

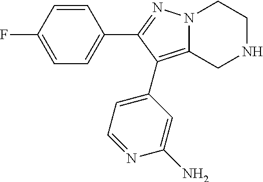 [SMILES: Nc1cc(-c2c(-c3ccc(F)cc3)nn3c2CNCC3)ccn1]

tert-Butyl 3-{2-[(tert-butoxycarbonyl)amino]pyridin-4-yl}-2-(4-fluorophenyl)-6,7-dihydropyrazolo[1,5-a]pyrazine-5(4H)-carboxylate (135 mg, 265 μmol, Intermediate 23) was added to a reaction vessel, followed by DCM (5.0 ml) and then the dropwise addition of trifluoroacetic acid (310 μl, 4.0 mmol; CAS-RN: [76-05-1]) and the mixture stirred at rt overnight. Additional trifluoroacetic acid (100 μl) was added and the mixture was stirred for a further 2 hours. DCM (50 ml) was added followed by NaHCO₃(saturated, aqueous 30 ml), the layer were separated, and the aqueous layer extracted with DCM (30 ml), the combined organic layers dried over sodium sulfate and concentrated under reduced pressure yielding the crude title compound (70.0 mg).

LC-MS (Method 3): Rₜ=0.48 min; MS (ESIpos): m/z=309 [M+H]⁺

### Intermediate 25

### tert-butyl 2-(4-fluorophenyl)-3-(2-methoxypyridin-4-yl)-6,7-dihydropyrazolo[1,5-a]pyrazine-5(4H)-carboxylate

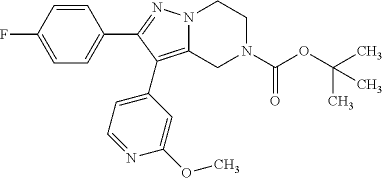 [SMILES: COc1cc(-c2c(-c3ccc(F)cc3)nn3c2CN(C(=O)OC(C)(C)C)CC3)ccn1]

tert-butyl 3-bromo-2-(4-fluorophenyl)-6,7-dihydropyrazolo[1,5-a]pyrazine-5(4H)-carboxylate (100 mg, 252 μmol, Intermediate 1), (2-methoxypyridin-4-yl) boronic acid (42.5 mg, 278 μmol, CAS-RN: [762262-09-9]), potassium carbonate (105 mg, 757 μmol; CAS-RN: [584-08-7]) and degassed THF (2.7 ml) and water (330 μl) were added to a microwave vessel. Pd(dppf)Cl₂ DCM complex (17.1 mg, 20.9 μmol; CAS-RN: [95464-05-4]) was added and the vessel was sealed under nitrogen. The mixture was stirred at 110° C. for 1 h in a microwave reactor. ethyl acetate (25 ml) was added, and the mixture was washed with water, NaHCO₃ solution and saturated sodium chloride solution (each 20 mL), the organic phase was dried over sodium sulfate, filtered and concentrated under reduced pressure, yielding the crude title compound (126 mg).

LC-MS (Method 3): Rₜ=1.38 min; MS (ESIpos): m/z=425.3 [M+H]⁺

### Intermediate 26

### 2-(4-fluorophenyl)-3-(2-methoxypyridin-4-yl)-4,5,6,7-tetrahydropyrazolo[1,5-a]pyrazine

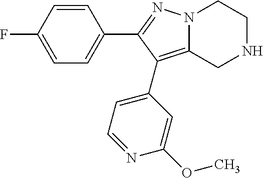 [SMILES: COc1cc(-c2c(-c3ccc(F)cc3)nn3c2CNCC3)ccn1]

To a solution of tert-butyl 2-(4-fluorophenyl)-3-(2-methoxypyridin-4-yl)-6,7-dihydropyrazolo[1,5-a]pyrazine-5(4H)-carboxylate (101 mg, 239 μmol, Intermediate 25) in DCM (2.0 ml) and added TFA (280 μl, 3.6 mmol; CAS-RN: [76-05-1]) under nitrogen. The mixture was stirred at rt for 3 h. The mixture was then diluted with DCM (15 ml) and sodium carbonate (aqueous, saturated, 15 ml), the layers separated and the aqueous layer extracted with DCM (20 ml). The combined organic layers were dried over sodium sulfate, filtered and concentrated under reduced pressure, yielding the crude title compound (98.7 mg).

LC-MS (Method 3): Rₜ=0.71 min; MS (ESIpos): m/z=325.1 [M+H]⁺

### Intermediate 27

### tert-butyl 2-(4-fluorophenyl)-3-(3-methoxypyridin-4-yl)-6,7-dihydropyrazolo[1,5-a]pyrazine-5(4H)-carboxylate

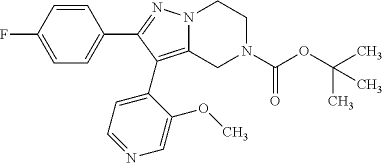 [SMILES: COc1cnccc1-c1c(-c2ccc(F)cc2)nn2c1CN(C(=O)OC(C)(C)C)CC2]

tert-butyl 3-bromo-2-(4-fluorophenyl)-6,7-dihydropyrazolo[1,5-a]pyrazine-5(4H)-carboxylate (100 mg, 252 μmol, Intermediate 1), 3-methoxy-4-(4,4,5,5-tetramethyl-1,3,2-dioxaborolan-2-yl)pyridine (65.3 mg, 278 μmol, CAS-RN: [1243312-43-7]), were added to a reaction vessel, followed by THF (2.7 ml), (105 mg, 757 μmol; CAS-RN: [584-08-7]) and water (330 μl). After degassing the mixture Pd(dppf)Cl₂ DCM complex (17.1 mg, 20.9 μmol; CAS-RN: [95464-05-4]) was added and the vessel was sealed under nitrogen. The mixture was stirred at 110° C. for 1 h in a microwave. Added ethyl acetate (25 ml), then washed with water, NaHCO₃ solution and saturated sodium chloride solution each 20 mL, dried organic phase over sodium sulfate, concentrated under reduced pressure yielding the crude title compound (146 mg).

LC-MS (Method 3): Rₜ=1.04 min; MS (ESIpos): m/z=425.3 [M+H]⁺

### Intermediate 28

### 2-(4-fluorophenyl)-3-(3-methoxypyridin-4-yl)-4,5,6,7-tetrahydropyrazolo[1,5-a]pyrazine

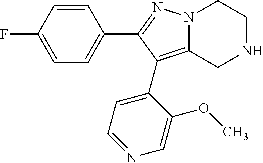 [SMILES: COc1cnccc1-c1c(-c2ccc(F)cc2)nn2c1CNCC2]

To a mixture of tert-butyl 2-(4-fluorophenyl)-3-(3-methoxypyridin-4-yl)-6,7-dihydropyrazolo[1,5-a]pyrazine-5(4H)-carboxylate (101 mg, 239 μmol, Intermediate 27) in DCM (2.0 ml) was added TFA (280 μl, 3.6 mmol; CAS-RN: [76-05-1]) under nitrogen. The mixture was stirred at rt for 3 h. The mixture was diluted with DCM (15 mL) and sat. sodium carbonate (15 mL) was added, the layers separated and the aqueous phase was extracted with DCM (2×20 ml), the combined organic phases dried over sodium sulfate, filtered and concentrated under reduced pressure yielding the crude title compound (95.0 mg).

LC-MS (Method 3): Rₜ=0.55 min; MS (ESIpos): m/z=325.2 [M+H]⁺

### Intermediate 29

### tert-butyl 2-(4-fluoroanilino)-6,7-dihydropyrazolo[1,5-a]pyrazine-5(4H)-carboxylate

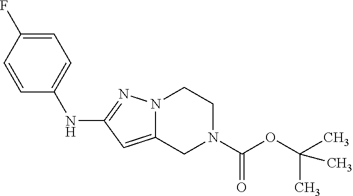 [SMILES: CC(C)(C)OC(=O)N1CCn2nc(Nc3ccc(F)cc3)cc2C1]

To a solution of tert-butyl 2-amino-6,7-dihydropyrazolo[1,5-a]pyrazine-5(4H)-carboxylate (228 mg, 957 μmol, CAS-RN: [1209487-56-8]) in 2 ml of 1,4-dioxane 1-bromo-4-fluorobenzene (335 mg, 1.91 mmol) was added. The resulting mixture was purged with argon for 5 min. Potassium carbonate (264 mg, 1.91 mmol; CAS-RN: [584-08-7]), Tris-(dibenzylidenacetone) dipalladium (35.0 mg, 38.3 μmol) and Xphos (18.2 mg, 38.3 μmol; CAS-RN: [564483-18-7]) were added and the mixture was heated to 100° C. for 18 h. After cooling to rt, 2 ml of water were added. The mixture was diluted with 5 ml of ethyl acetate and filtered through a pad of celite. The layers were separated and the aqueous layer was extracted with ethyl acetate. The combined organic layers were dried over sodium sulfate, filtered and the solvent was evaporated under reduced pressure. The residue was purified by silica gel column chromatography (eluent: ethyl acetate/hexane 0-90%) yielding 135 mg (99% purity, 42% yield) of the title compound.

LC-MS (Method 4): Rₜ=1.91 min; MS (ESIpos): m/z=333.2 [M+H]⁺

¹H-NMR (600 MHz, DMSO-d6) δ ppm 1.42 (s, 9H) 3.74-3.85 (m, 2H) 3.90-3.99 (m, 2H) 4.53 (br s, 2H) 5.65 (s, 1H) 6.96-7.06 (m, 2H) 7.31-7.40 (m, 2H) 8.45 (s, 1H).

### Intermediate 30

### tert-butyl 3-bromo-2-(4-fluoroanilino)-6,7-dihydropyrazolo[1,5-a]pyrazine-5(4H)-carboxylate

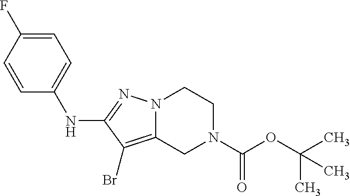 [SMILES: CC(C)(C)OC(=O)N1CCn2nc(Nc3ccc(F)cc3)c(Br)c2C1]

To a solution of tert-butyl 2-(4-fluoroanilino)-6,7-dihydropyrazolo[1,5-a]pyrazine-5(4H)-carboxylate (135 mg, 406 μmol, Intermediate 29) in 2 ml DCM 1-bromopyrrolidine-2,5-dione (72.3 mg, 406 μmol; CAS-RN: [128-08-5]) was added at 0° C. in one portion. The ice bath was removed and the stirred mixture was allowed to warm to rt. After 30 min NaHCO₃(saturated, aqueous, 1 ml) and DCM (5 ml) were added. The mixture was dried with an water-repellent filter and concentrated under reduced pressure. The residue was purified by silica gel column chromatography (eluent: ethyl acetate/hexane 0-90%) yielding 108 mg (94% purity, 61% yield) of the title compound.

LC-MS (Method 4): Rₜ=2.22 min; MS (ESIpos): m/z=411 [M+H]⁺

¹H NMR (600 MHz, DMSO-d₆) δ ppm 1.44 (s, 9H) 3.78-3.86 (m, 2H) 3.95-4.03 (m, 2H) 4.45 (br s, 2H) 6.97-7.06 (m, 2H) 7.39-7.47 (m, 2H) 7.95 (s, 1H)

### Intermediate 31

### tert-butyl 2-(4-fluoroanilino)-3-(pyridin-4-yl)-6,7-dihydropyrazolo[1,5-a]pyrazine-5(4H)-carboxylate

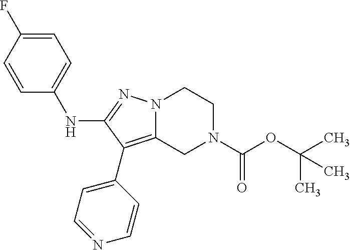 [SMILES: CC(C)(C)OC(=O)N1CCn2nc(Nc3ccc(F)cc3)c(-c3ccncc3)c2C1]

reaction vessel, tert-butyl 3-bromo-2-(4-fluoroanilino)-6,7-In a microwave dihydropyrazolo[1,5-a]pyrazine-5(4H)-carboxylate (78.0 mg, 190 μmol, Intermediate 30) und pyridin-4-ylboronic acid (25.6 mg, 209 μmol) were dissolved in 1,4-dioxane (2 ml) and were flushed with Argon. With stirring, PdCl2(PCy3)2 (14.0 mg, 19.0 μmol; CAS-RN: [29934-17-6]) and aqueous sodium carbonate solution (460 μl, 2.0 M, 910 μmol) were added, the vial was sealed and the mixture was heated at 150° C. for 30 min in a microwave. After cooling to rt, ethyl acetate and water was added and the mixture was filtered through a pad of celite. The layers were separated and the aqueous layer was extracted with ethyl acetate. The combined organic layers were dried over sodium sulfate, filtered and the solvent was evaporated under reduced pressure. The residue was purified by reverse phase preparative HPLC (Method 5) yielding 17.0 mg (97% purity, 21% yield) of the desired product.

LC-MS (Method 4): Rₜ=1.36 min; MS (ESIneg): m/z=408 [M−H]⁻

¹H NMR (600 MHz, DMSO-d₆) δ ppm 1.42 (br s, 9H) 3.83-3.91 (m, 2H) 4.01-4.11 (m, 2H) 4.69 (br s, 2H) 6.91-7.02 (m, 2H) 7.17-7.26 (m, 2H) 7.31-7.38 (m, 2H) 8.02 (s, 1H) 8.51-8.57 (m, 2H)

### Intermediate 32

### N-(4-fluorophenyl)-3-(pyridin-4-yl)-4,5,6,7-tetrahydropyrazolo[1,5-a]pyrazin-2-amine

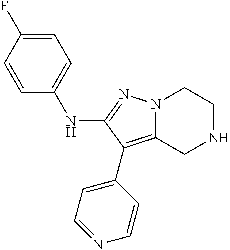 [SMILES: Fc1ccc(Nc2nn3c(c2-c2ccncc2)CNCC3)cc1]

To a solution of tert-butyl 2-(4-fluoroanilino)-3-(pyridin-4-yl)-6,7-dihydropyrazolo[1,5-a]pyrazine-5(4H)-carboxylate (16.0 mg, 39.1 μmol, Intermediate 31) in DCM (4 ml) (30 μl, 390 μmol) TFA was added. The mixture was stirred at rt for 18 h. An additional amount of (30 μl, 390 μmol) TFA was added and stirring at rt was continued for 24 h. DCM (5 ml) was added followed by NaHCO₃(saturated, aqueous 1 ml). The mixture was dried with an water-repellent filter and concentrated under reduced pressure. The crude product 6.30 mg (73% purity, 38% yield) was used without further purification.

LC-MS (Method 4): Rₜ=0.45 min; MS (ESIpos): m/z=310 [M+H]⁺

¹H NMR (600 MHz, DMSO-d6) δ ppm 3.11-3.19 (m, 2H) 3.90-3.94 (m, 2H) 3.98 (br s, 2H) 6.90-7.03 (m, 2H) 7.13-7.23 (m, 2H) 7.28-7.37 (m, 2H) 7.94 (s, 1H) 8.45-8.53 (m, 2H)

### Intermediate 33

### tert-butyl 3-(2-anilinopyridin-4-yl)-2-(4-fluorophenyl)-6,7-dihydropyrazolo[1,5-a]pyrazine-5(4H)-carboxylate

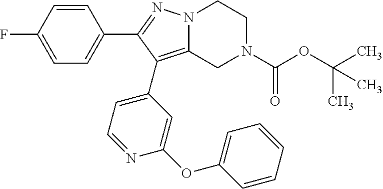 [SMILES: CC(C)(C)OC(=O)N1CCn2nc(-c3ccc(F)cc3)c(-c3ccnc(Oc4ccccc4)c3)c2C1]

tert-butyl 3-bromo-2-(4-fluorophenyl)-6,7-dihydropyrazolo[1,5-a]pyrazine-5(4H)-carboxylate (200 mg, 505 μmol, Intermediate 1) were added to a mixture of THF (5.3 ml) and water (690 μl) in a microwave reaction vessel. The reaction vessel flushed with nitrogen and N-phenyl-4-(4,4,5,5-tetramethyl-1,3,2-dioxaborolan-2-yl)pyridin-2-amine (164 mg, 555 μmol), potassium carbonate (209 mg, 1.51 mmol; CAS-RN: [584-08-7]) and dichloro[1,1′-bis(dialkyl/diarylphosphino) ferrocene]palladium (II) (34.2 mg, 41.9 μmol; CAS-RN: [95464-05-4]) were added. The sealed vessel was heated at 110° C. for 1 hour in a microwave. ethyl acetate and water was added and the mixture neutralized using 1M acid acid. After extraction 2× with ethyl acetate the combined organic phases were dried with an water-repellent filter and concentrated under reduced pressure. The residue was purified by silica gel column chromatography (eluent: hexane/ethyl acetate 0-20%) yielding the title compound 109 mg (93% purity, 41% yield).

LC-MS (Method 1): Rₜ=1.06 min; MS (ESIpos): m/z=486.1 [M+H]⁺

¹H-NMR (400 MHz, DMSO-d6) δ [ppm]: 1.066 (16.00), 1.154 (1.07), 1.172 (2.00), 1.190 (0.94), 1.423 (1.73), 1.988 (3.68), 2.518 (0.96), 2.523 (0.64), 3.939 (2.57), 4.017 (0.73), 4.035 (0.73), 4.651 (0.96), 7.194 (0.48), 7.197 (0.52), 7.215 (0.69), 7.220 (0.92), 7.234 (0.46), 7.242 (0.48).

### Intermediate 34

### trifluoroacetic acid-4-[2-(4-fluorophenyl)-4,5,6,7-tetrahydropyrazolo[1,5-a]pyrazin-3-yl]-N-phenylpyridin-2-amine (1/1)

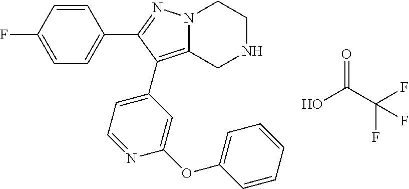 [SMILES: Fc1ccc(-c2nn3c(c2-c2ccnc(Oc4ccccc4)c2)CNCC3)cc1 ; O=C(O)C(F)(F)F]

tert-butyl 3-(2-anilinopyridin-4-yl)-2-(4-fluorophenyl)-6,7-dihydropyrazolo[1,5-a]pyrazine-5(4H)-carboxylate (105 mg, 216 μmol, Intermediate 33) was added to a reaction vessel, followed by DCM (2.0 ml) and then the dropwise addition of trifluoroacetic acid (170 μl, 2.2 mmol; CAS-RN: [76-05-1]). The mixture was stirred at rt for 5 hours. The solution was concentrated under reduced pressure to yield 84 mg (95% purity, 74% yield) of the title crude product which was used without further purification in the next reaction.

LC-MS (Method 2): Rₜ=1.10 min; MS (ESIpos): m/z=386.5 [M+H]⁺

### Intermediate 35

### tert-butyl 2-(4-fluorophenyl)-3-(1H-pyrrolo[2,3-b]pyridin-4-yl)-6,7-dihydropyrazolo[1,5-a]pyrazine-5(4H)-carboxylate

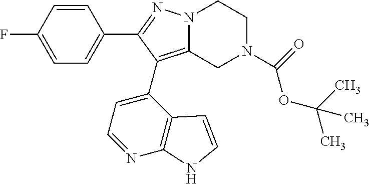 [SMILES: CC(C)(C)OC(=O)N1CCn2nc(-c3ccc(F)cc3)c(-c3ccnc4[nH]ccc34)c2C1]

tert-butyl 3-bromo-2-(4-fluorophenyl)-6,7-dihydropyrazolo[1,5-a]pyrazine-5(4H)-carboxylate (148 mg, 373 μmol, Intermediate 1) were added to a mixture of THF (3.9 ml) and water (510 μl) in a microwave reaction vessel. The reaction vessel flushed with nitrogen and 1H-pyrrolo[2,3-b]pyridin-4-ylboronic acid (96.8 mg, 598 μmol), potassium carbonate (155 mg, 1.12 mmol; CAS-RN: [584-08-7]) and dichloro[1,1′-bis(dialkyl/diarylphosphino)-ferrocene]palladium (II) (25.3 mg, 31 μmol; CAS-RN: [95464-05-4]) were added. The sealed vessel was heated at 110° C. for 1 hour in a microwave. ethyl acetate and water was added and the mixture neutralized using 1M acid acid. After extraction 2× with ethyl acetate the combined organic phases were dried with an water-repellent filter and concentrated under reduced pressure. The residue was purified by silica gel column chromatography (eluent: hexane/ethyl acetate 0-100%) yielding the title compound 45 mg (28% yield).

LC-MS (Method 2): Rₜ=1.22 min; MS (ESIpos): m/z=435 [M+H]⁺

### Intermediate 36

### trifluoroacetic acid-2-(4-fluorophenyl)-3-(1H-pyrrolo[2,3-b]pyridin-4-yl)-4,5,6,7-tetrahydropyrazolo[1,5-a]pyrazine (1/1)

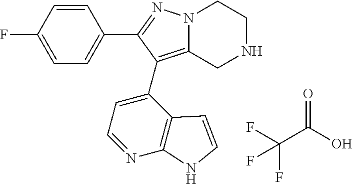

tert-butyl 2-(4-fluorophenyl)-3-(1H-pyrrolo[2,3-b]pyridin-4-yl)-6,7-dihydropyrazolo[1,5-a]-pyrazine-5(4H)-carboxylate (85.0 mg, 196 μmol, Intermediate 35) was added to a reaction vessel, followed by DCM (2.0 ml) and then the dropwise addition of trifluoroacetic acid (150 μl, 2.0 mmol; CAS-RN: [76-05-1]). The mixture was stirred at rt for 3 hours. The solution was concentrated under reduced pressure to yield 65.0 mg (90% purity, 67% yield) of the title crude product which was used without further purification in the next reaction.

LC-MS (Method 2): Rₜ=0.85 min; MS (ESIpos): m/z=334.3 [M+H]⁺

### Intermediate 37

### ethyl 3-bromo-1-{2-[(tert-butoxycarbonyl)amino]ethyl}-1H-pyrazole-5-carboxylate

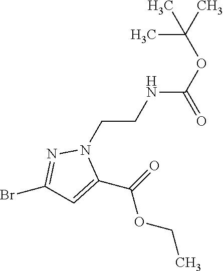 [SMILES: CCOC(=O)c1cc(Br)nn1CCNC(=O)OC(C)(C)C]

To a solution of ethyl 5-bromo-1H-pyrazole-3-carboxylate (10.0 g, 45.7 mmol CAS-RN: [1392208-46-6), tert-butyl(2-hydroxyethyl) carbamate (7.36 g, 45.7 mmol) and triphenylphosphine (23.9 g, 91.3 mmol) in tetrahydrofuran (80 ml) was added diisopropyl azodicarboxylate (18.5 g, 91.3 mmol) at 20° C. The resulting mixture was stirred at 20° C. for 10 h. The reaction solution was concentrated in vacuo to give ethyl 3-bromo-1-{2-[(tert-butoxycarbonyl)amino]ethyl}-1H-pyrazole-5-carboxylate (16.0 g, 44.2 mmol, 97% yield) as a yellow oil.

LC-MS (Method 11): Rₜ=0.842 min; MS (ESIpos): m/z=262.0 [(M−100)+H]⁺, 306.0 [(M−55)+H]⁺.

### Intermediate 38

### tert-butyl(2-(3-bromo-5-(hydroxymethyl)-1H-pyrazol-1-yl)ethyl) carbamate

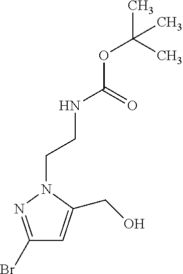 [SMILES: CC(C)(C)OC(=O)NCCn1nc(Br)cc1CO]

To a solution of ethyl 3-bromo-1-{2-[(tert-butoxycarbonyl)amino]ethyl}-1H-pyrazole-5-carboxylate (16.0 g, 44.2 mmol, Intermediate 37) in ethanol (400 ml) was added sodium tetrahydroborate (5.01 g, 133 mmol) in batches under 0° C. After the addition, the reaction mixture was allowed to warm to 20° C. and stirred for 12 hours. The reaction mixture was quenched with slowly adding water, and then concentrated under vacuo to give a crude. The crude was triturated with ethanol, filtered and the filtrate was concentrated to give a residue. The residue was diluted with water and extracted with ethyl acetate. The combined organic layers were washed with brine, dried over anhydrous sodium sulfate, filtered and concentrated in vacuo to give tert-butyl(2-(3-bromo-5-(hydroxymethyl)-1H-pyrazol-1-yl)ethyl) carbamate (14.0 g, 43.7 mmol, 99% yield) as a yellow solid.

LC-MS (Method 11): Rₜ=0.503 min; MS (ESIpos): m/z=320.2 [M+H]⁺.

### Intermediate 39

### ethyl 1-(2-aminoethyl)-3-bromo-1H-pyrazole-5-carboxylate

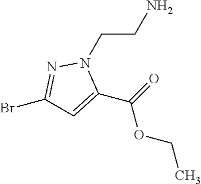

To a solution of ethyl 3-bromo-1-{2-[(tert-butoxycarbonyl)amino]ethyl}-1H-pyrazole-5-carboxylate (13.0 g, 35.9 mmol, Intermediate 37) in ethyl acetate (43 ml) was added hydrochloric acid (4 M in ethyl acetate, 27 ml, 110 mmol) at 20° C. The mixture was stirred at 20° C. for 1 hour. The reaction mixture was filtered and the solid cake was collected to give a crude product. The crude product was diluted with water and the resulting mixture was filtered. The filtrate was basified with saturated sodium bicarbonate aqueous solution and then extracted with ethyl acetate. The combined organic layers were washed with brine, dried over anhydrous sodium sulfate, filtered and concentrated in vacuo to give the title compound as a white solid (8.6 g, 91% yield).

### Intermediate 40

### 2-bromo-6,7-dihydropyrazolo[1,5-a]pyrazin-4 (5H)-one

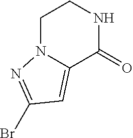 [SMILES: O=C1NCCn2nc(Br)cc21]

To a solution of ethyl 1-(2-aminoethyl)-3-bromo-1H-pyrazole-5-carboxylate (8.60 g, 32.8 mmol, Intermediate 39) in methanol (150 ml) was added triethylamine (13.7 ml, 98.4 mmol) at 20° C. The mixture was stirred at 50° C. for 12 hours. The reaction mixture was concentrated in vacuo to give 2-bromo-6,7-dihydropyrazolo[1,5-a]pyrazin-4 (5H)-one (7 g, 32.4 mmol, 99% yield) as a white solid.

LC-MS (Method 11): Rₜ=0.224 min; MS (ESIpos): m/z=216.0 [M+H]⁺

### Intermediate 41

### 2-(4-chloro-3-fluorophenyl)-6,7-dihydropyrazolo[1,5-a]pyrazin-4 (5H)-one

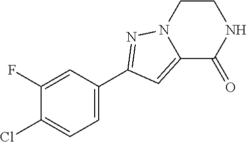 [SMILES: O=C1NCCn2nc(-c3ccc(Cl)c(F)c3)cc21]

To a mixture of 2-bromo-6,7-dihydropyrazolo[1,5-a]pyrazin-4 (5H)-one (450 mg, 2.08 mmol, Intermediate 40) and (4-chloro-3-fluorophenyl) boronic acid (710 mg, 4.07 mmol) in 1,4-dioxane (10 ml) and water (3 ml) were added dichloro[1,1-bis(diphenylphosphino) ferrocene]palladium (II) (270 mg, 0.370 mmol) and sodium carbonate (784 mg, 7.40 mmol) at 20° C. The resulting mixture was stirred at 90° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give 2-(4-chloro-3-fluorophenyl)-6,7-dihydropyrazolo[1,5-a]pyrazin-4 (5H)-one (950 mg, 3.57 mmol, 96% yield) as a brown solid.

LC-MS (Method 11): Rₜ=0.856 min; MS (ESIpos): m/z=266.0 [M+H]⁺

### Intermediate 42

### 2-(4-chloro-3-fluorophenyl)-4,5,6,7-tetrahydropyrazolo[1,5-a]pyrazine

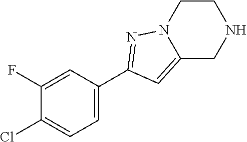 [SMILES: Fc1cc(-c2cc3n(n2)CCNC3)ccc1Cl]

To a solution of 2-(4-chloro-3-fluorophenyl)-6,7-dihydropyrazolo[1,5-a]pyrazin-4 (5H)-one (950 mg, 3.57 mmol, Intermediate 41) in tetrahydrofuran (20 ml) was added borane dimethyl sulfide complex (10 ml, 10 M in tetrahydrofuran) at 20° C. and the resulting solution was stirred at 65° C. for 12 hours. The reaction was quenched with methanol and the resulting solution was concentrated in vacuo to give 2-(4-chloro-3-fluorophenyl)-4,5,6,7-tetrahydropyrazolo[1,5-a]pyrazine (850 mg, 3.37 mmol, 94% yield) as a black oil.

LC-MS (Method 11): Rₜ=0.657 min; MS (ESIpos): m/z=252.0 [M+H]⁺.

### Intermediate 43

### tert-butyl 2-(4-chloro-3-fluorophenyl)-6,7-dihydropyrazolo[1,5-a]pyrazine-5(4H)-carboxylate

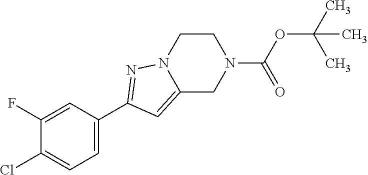 [SMILES: CC(C)(C)OC(=O)N1CCn2nc(-c3ccc(Cl)c(F)c3)cc2C1]

To a solution of 2-(4-chloro-3-fluorophenyl)-4,5,6,7-tetrahydropyrazolo[1,5-a]pyrazine (850 mg, 3.37 mmol, Intermediate 42) in dichloromethane (10 ml) were added trimethylamine (1.4 ml, 10.1 mmol), 4-(dimethylamino)pyridine (41.0 mg, 0.337 mmol) and di-tert-butyl dicarbonate (1.5 ml, 6.75 mmol) at 20° C. The resulting mixture was stirred at 20° C. for 4 hours. The reaction mixture was poured into water and extracted with ethyl acetate. The combined organic layers were washed with brine, dried over anhydrous sodium sulfate, filtered and concentrated in vacuo to give tert-butyl 2-(4-chloro-3-fluorophenyl)-6,7-dihydropyrazolo[1,5-a]pyrazine-5(4H)-carboxylate (1000 mg, 2.84 mmol, 84% yield) as a brown solid.

LC-MS (Method 11): Rₜ=1.029 min; MS (ESIpos): m/z=352.1 [M+H]⁺

### Intermediate 44

### tert-butyl 2-(4-chloro-3-fluorophenyl)-3-iodo-6,7-dihydropyrazolo[1,5-a]pyrazine-5(4H)-carboxylate

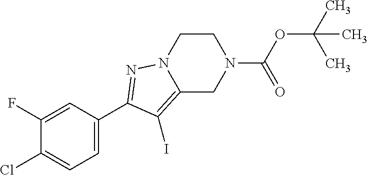 [SMILES: CC(C)(C)OC(=O)N1CCn2nc(-c3ccc(Cl)c(F)c3)c(I)c2C1]

To a solution of tert-butyl 2-(4-chloro-3-fluorophenyl)-6,7-dihydropyrazolo[1,5-a]pyrazine-5(4H)-carboxylate (100 mg, 2.84 mmol, Intermediate 43) in dichloromethane (10 ml) was added N-iodosuccinimide (703 mg, 3.12 mmol) at 20° C. The reaction mixture was stirred at 43° C. for 3 hours. The reaction mixture was poured into sodium sulfite aqueous solution and extracted with ethyl acetate. The combined organic layers were washed with brine, dried over anhydrous sodium sulfate, filtered and concentrated in vacuo to give tert-butyl 2-(4-chloro-3-fluorophenyl)-3-iodo-6,7-dihydropyrazolo[1,5-a]pyrazine-5(4H)-carboxylate (1.10 g, 2.30 mmol, 81% yield) as a black solid.

LC-MS (Method 11): Rₜ=1.106 min; MS (ESIpos): m/z=478.0 [M+H]⁺.

### Intermediate 45

### tert-butyl 2-(4-chloro-3-fluorophenyl)-3-(pyridin-4-yl)-6,7-dihydropyrazolo[1,5-a]pyrazine-5(4H)-carboxylate

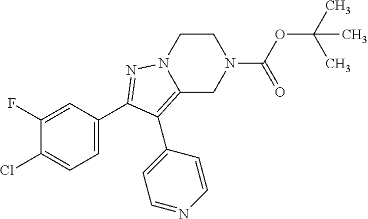 [SMILES: CC(C)(C)OC(=O)N1CCn2nc(-c3ccc(Cl)c(F)c3)c(-c3ccncc3)c2C1]

To a mixture of tert-butyl 2-(4-chloro-3-fluorophenyl)-3-iodo-6,7-dihydropyrazolo[1,5-a]pyrazine-5(4H)-carboxylate (1100 mg, 2.30 mmol, Intermediate 44) and pyridin-4-ylboronic acid (283 mg, 2.30 mmol) in 1,4-dioxane (50 ml) and water (10 ml) were added dichloro[1,1-bis(diphenylphosphino) ferrocene]palladium (II) (153 mg, 0.209 mmol) and sodium carbonate (444 mg, 4.18 mmol) at 20° C. The resulting mixture was stirred at 80° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a crude product. The crude product was purified by column chromatography on silica gel (petroleum ether:ethyl acetate=1:1) to give tert-butyl 2-(4-chloro-3-fluorophenyl)-3-(pyridin-4-yl)-6,7-dihydropyrazolo[1,5-a]pyrazine-5(4H)-carboxylate (511 mg, 1.19 mmol, 56% yield) as a gray solid.

LC-MS (Method 11): Rₜ=0.857 min; MS (ESIpos): m/z=429.1 [M+H]⁺

### Intermediate 46

### 2-(4-chloro-3-fluorophenyl)-3-(pyridin-4-yl)-4,5,6,7-tetrahydropyrazolo[1,5-a]pyrazine hydrochloride (1:1)

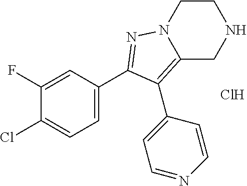 [SMILES: Fc1cc(-c2nn3c(c2-c2ccncc2)CNCC3)ccc1Cl ; Cl]

To a solution of tert-butyl 2-(4-chloro-3-fluorophenyl)-3-(pyridin-4-yl)-6,7-dihydropyrazolo[1,5-a]pyrazine-5(4H)-carboxylate (511 mg, 1.19 mmol, Intermediate 45) in ethyl acetate (10 ml) was added hydrochloric acid (1.4 ml, 4 M in ethyl acetate) at 20° C. The resulting mixture was stirred at 20° C. for 2 hours. The reaction mixture was filtered and the solid cake was collected to give 2-(4-chloro-3-fluorophenyl)-3-(pyridin-4-yl)-4,5,6,7-tetrahydropyrazolo[1,5-a]pyrazine hydrochloride (1:1) (390 mg, 1.06 mmol, 89% yield) as a yellow solid.

LC-MS (Method 11): Rₜ=0.495 min; MS (ESIpos): m/z=329.1 [M+H]⁺

### Intermediate 47

### 2-(4-chloro-2-fluorophenyl)-6,7-dihydropyrazolo[1,5-a]pyrazin-4 (5H)-one

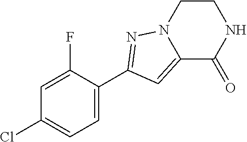 [SMILES: O=C1NCCn2nc(-c3ccc(Cl)cc3F)cc21]

To a solution of 2-bromo-6,7-dihydropyrazolo[1,5-a]pyrazin-4 (5H)-one (600 mg, 2.78 mmol, Intermediate 40) and (4-chloro-2-fluorophenyl) boronic acid (533 mg, 3.06 mmol) in 1,4-dioxane (10 ml) and water (2 ml) were added sodium carbonate (589 mg, 5.55 mmol) and (1, 1′-bis(diphenylphosphino) ferrocene) palladium (II) dichloride (203 mg, 278 μmol) at 20° C. The mixture was degassed with nitrogen and stirred at 90° C. for 16 hours. The reaction mixture was poured into water and extracted with ethyl acetate. Combined extracts were dried over anhydrous sodium sulfate, filtered and the filtrate was concentrated under vacuum to give 2-(4-chloro-2-fluorophenyl)-6,7-dihydropyrazolo[1,5-a]pyrazin-4 (5H)-one (500 mg, 1.88 mmol, 68% yield) as a brown solid.

LC-MS (Method 11): Rₜ=0.752 min; MS (ESIpos): m/z=266.0 [M+H]⁺

### Intermediate 48

### 2-(4-chloro-2-fluorophenyl)-4,5,6,7-tetrahydropyrazolo[1,5-a]pyrazine

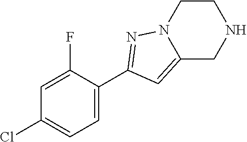 [SMILES: Fc1cc(Cl)ccc1-c1cc2n(n1)CCNC2]

To a solution of 2-(4-chloro-2-fluorophenyl)-6,7-dihydropyrazolo[1,5-a]pyrazin-4 (5H)-one (500 mg, 1.88 mmol, Intermediate 47) in tetrahydrofuran (20 ml) was added borane tetrahydrofuran complex (11 ml, 11 mmol, 1.0 M in tetrahydrofuran) dropwise via syringe at 0° C. under nitrogen atmosphere. The mixture was stirred at 65° C. for 12 hours. The reaction mixture was quenched with methanol and the mixture was concentrated under vacuum to give 2-(4-chloro-2-fluorophenyl)-4,5,6,7-tetrahydropyrazolo[1,5-a]pyrazine (470 mg, 1.87 mmol, 99% yield) as a black gum.

LC-MS (Method 11): Rₜ=0.808 min; MS (ESIpos): m/z=252.1 [M+H]⁺

### Intermediate 49

### tert-butyl 2-(4-chloro-2-fluorophenyl)-6,7-dihydropyrazolo[1,5-a]pyrazine-5(4H)-carboxylate

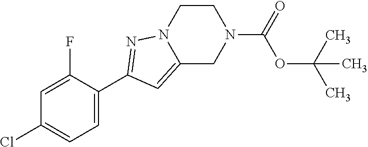 [SMILES: CC(C)(C)OC(=O)N1CCn2nc(-c3ccc(Cl)cc3F)cc2C1]

To a solution of 2-(4-chloro-2-fluorophenyl)-4,5,6,7-tetrahydropyrazolo[1,5-a]pyrazine (470 mg, 1.87 mmol, Intermediate 48) in dichloromethane (20 ml) were added di-tert-butyl dicarbonate (22.8 mg, 187 μmol), triethylamine (780 μl, 5.6 mmol) and 4-(dimethylamino)pyridine (22.8 mg, 187 μmol) at 20° C. The mixture was stirred at 20° C. for 2 hours. The reaction mixture was concentrated under vacuum to give a crude product and the crude product product was purified by silica gel column chromatography (petroleum ether:ethyl aceate=1:0 to 3:1) to give tert-butyl 2-(4-chloro-2-fluorophenyl)-6,7-dihydropyrazolo[1,5-a]pyrazine-5(4H)-carboxylate (500 mg, 1.42 mmol, 76% yield) as a yellow solid.

LC-MS (Method 11): Rₜ=0.929 min; MS (ESIpos): m/z=352.1 [M+H]⁺

### Intermediate 50

### tert-butyl 2-(4-chloro-2-fluorophenyl)-3-iodo-6,7-dihydropyrazolo[1,5-a]pyrazine-5(4H)-carboxylate

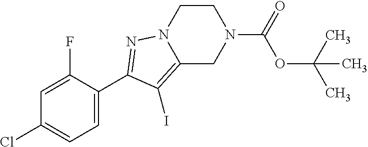 [SMILES: CC(C)(C)OC(=O)N1CCn2nc(-c3ccc(Cl)cc3F)c(I)c2C1]

To a solution of tert-butyl 2-(4-chloro-2-fluorophenyl)-6,7-dihydropyrazolo[1,5-a]pyrazine-5(4H)-carboxylate (500 mg, 1.42 mmol, Intermediate 49) in dichloromethane (10 ml) and methanol (2.5 ml) was added 1-iodopyrrolidine-2,5-dione (640 mg, 2.84 mmol) in partions at 20° C. and the reaction mixture was stirred at 43° C. for 16 hours. The reaction mixture was quenched with saturated sodium sulfite aqueous solution and the resulting mixture was extracted with ethyl acetate. Combined extracts were dried over anhydrous sodium sulfate, filtered and the filtrate was concentrated under vacuum to give tert-butyl 2-(4-chloro-2-fluorophenyl)-3-iodo-6,7-dihydropyrazolo[1,5-a]pyrazine-5(4H)-carboxylate (500 mg, 1.05 mmol, 74% yield) as a yellow oil.

LC-MS (Method 11): Rₜ=1.105 min; MS (ESIpos): m/z=478.1 [M+H]⁺

### Intermediate 51

### tert-butyl 2-(4-chloro-2-fluorophenyl)-3-(pyridin-4-yl)-6,7-dihydropyrazolo[1,5-a]pyrazine-5(4H)-carboxylate

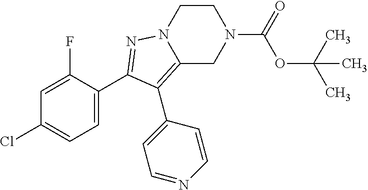 [SMILES: CC(C)(C)OC(=O)N1CCn2nc(-c3ccc(Cl)cc3F)c(-c3ccncc3)c2C1]

To a solution of tert-butyl 2-(4-chloro-2-fluorophenyl)-3-iodo-6,7-dihydropyrazolo[1,5-a]pyrazine-5(4H)-carboxylate (480 mg, 1.00 mmol, Intermediate 50) and pyridin-4-ylboronic acid (247 mg, 2.01 mmol) in 1,4-dioxane (9 ml) and water (2 ml) were added sodium carbonate (213 mg, 2.01 mmol) and (1,1′-bis(diphenylphosphino) ferrocene) palladium (II) dichloride (60.7 mg, 100 μmol) at 20° C. The mixture was degassed with nitrogen and stirred at 80° C. for 16 hours. The reaction mixture was concentrated under vacuum to give a crude product product and the crude product was purified by silica gel column chromatography (petroleum ether:ethyl aceate=1:0 to 1:1) to give tert-butyl 2-(4-chloro-2-fluorophenyl)-3-(pyridin-4-yl)-6,7-dihydropyrazolo[1,5-a]pyrazine-5(4H)-carboxylate (270 mg, 630 μmol, 63% yield) as a yellow oil.

LC-MS (Method 11): Rₜ=0.841 min; MS (ESIpos): m/z=429.2 [M+H]⁺.

### Intermediate 52

### 2-(4-chloro-2-fluorophenyl)-3-(pyridin-4-yl)-4,5,6,7-tetrahydropyrazolo[1,5-a]pyrazine hydrogen chloride

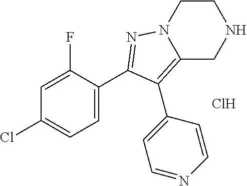 [SMILES: Fc1cc(Cl)ccc1-c1nn2c(c1-c1ccncc1)CNCC2 ; Cl]

To a solution of hydrochloric acid (459 mg, 12.6 mmol) in ethyl aceate (10 ml) was added tert-butyl 2-(4-chloro-2-fluorophenyl)-3-(pyridin-4-yl)-6,7-dihydropyrazolo[1,5-a]pyrazine-5(4H)-carboxylate (270 mg, 630 μmol, Intermediate 51) at 20° C. and the reaction mixture was stirred at 20° C. for 1 hour. The reaction mixture was concentrated under vacuum to give 2-(4-chloro-2-fluorophenyl)-3-(pyridin-4-yl)-4,5,6,7-tetrahydropyrazolo[1,5-a]pyrazine hydrogen chloride (220 mg, 602 μmol, 96% yield) as a yellow solid.

LC-MS (Method 14): Rₜ=0.641 min; MS (ESIpos): m/z=329.1 [M+H]⁺

### Intermediate 53

### 2-(3-chloro-4-methoxyphenyl)-6,7-dihydropyrazolo[1,5-a]pyrazin-4 (5H)-one

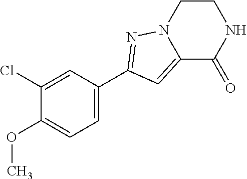 [SMILES: COc1ccc(-c2cc3n(n2)CCNC3=O)cc1Cl]

To a mixture of 2-bromo-6,7-dihydropyrazolo[1,5-a]pyrazin-4 (5H)-one (800 mg, 3.70 mmol, Intermediate 40) and (3-chloro-4-methoxyphenyl) boronic acid (759 mg, 4.07 mmol) in 1,4-dioxane (10 ml) and water (3 ml) were added dichloro[1,1-bis(diphenylphosphino)-ferrocene]palladium (II) (270 mg, 0.370 mmol) and sodium carbonate (784 mg, 7.40 mmol) at 20° C. The mixture was stirred at 90° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give 2-(3-chloro-4-methoxyphenyl)-6,7-dihydropyrazolo[1,5-a]pyrazin-4 (5H)-one (1.00 g, 3.60 mmol, 97% yield) as a gray solid.

LC-MS (Method 11): Rₜ=0.819 min; MS (ESIpos): m/z=278.0 [M+H]⁺

### Intermediate 54

### 2-(3-chloro-4-methoxyphenyl)-4,5,6,7-tetrahydropyrazolo[1,5-a]pyrazine

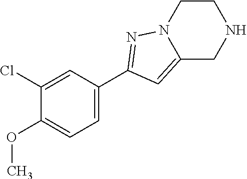 [SMILES: COc1ccc(-c2cc3n(n2)CCNC3)cc1Cl]

To a solution of 2-(3-chloro-4-methoxyphenyl)-6,7-dihydropyrazolo[1,5-a]pyrazin-4 (5H)-one (1000 mg, 3.60 mmol, Intermediate 53) in tetrahydrofuran (20 ml) was added borane dimethyl sulfide complex (10 ml, 10 M in tetrahydrofuran) at 20° C. and the resulting solution was stirred at 65° C. for 12 hours. The reaction was quenched with methanol and the resulting solution was concentrated in vacuo to give 2-(3-chloro-4-methoxyphenyl)-4,5,6,7-tetrahydropyrazolo[1,5-a]pyrazine (900 mg, 3.41 mmol, 94% yield) as a black oil.

LC-MS (Method 11): Rₜ=0.565 min; MS (ESIpos): m/z=264.0 [M+H]⁺

### Intermediate 55

### tert-butyl 2-(3-chloro-4-methoxyphenyl)-6,7-dihydropyrazolo[1,5-a]pyrazine-5(4H)-carboxylate

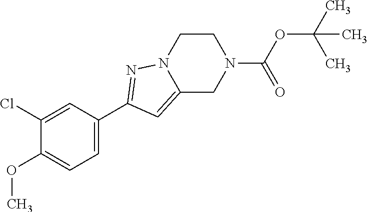 [SMILES: COc1ccc(-c2cc3n(n2)CCN(C(=O)OC(C)(C)C)C3)cc1Cl]

To a solution of 2-(4-chloro-3-fluorophenyl)-4,5,6,7-tetrahydropyrazolo[1,5-a]pyrazine (900 mg, 3.41 mmol, Intermediate 54) in dichloromethane (10 ml) were added trimethylamine (1.4 ml, 10.2 mmol), 4-(dimethylamino)pyridine (42.0 mg, 0.341 mmol) and di-tert-butyl dicarbonate (1.5 ml, 6.82 mmol) at 20° C. The resulting mixture was stirred at 20° C. for 2 hours. The reaction mixture was poured into water and extracted with ethyl acetate. The combined organic layers were washed with brine, dried over anhydrous sodium sulfate, filtered and concentrated in vacuo to give tert-butyl 2-(3-chloro-4-methoxyphenyl)-6,7-dihydropyrazolo[1,5-a]pyrazine-5(4H)-carboxylate (1.10 g, 3.02 mmol, 88% yield) as a black oil.

LC-MS (Method 11): Rₜ=0.992 min; MS (ESIpos): m/z=364.1 [M+H]⁺

### Intermediate 56

### tert-butyl 2-(3-chloro-4-methoxyphenyl)-3-iodo-6,7-dihydropyrazolo[1,5-a]pyrazine-5(4H)-carboxylate

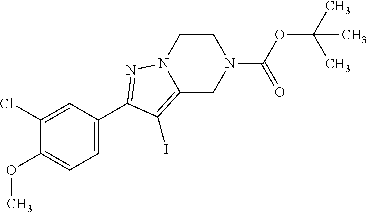 [SMILES: COc1ccc(-c2nn3c(c2I)CN(C(=O)OC(C)(C)C)CC3)cc1Cl]

To a solution of tert-butyl 2-(3-chloro-4-methoxyphenyl)-6,7-dihydropyrazolo[1,5-a]pyrazine-5(4H)-carboxylate (1100 mg, 3.02 mmol, Intermediate 55) in dichloromethane (10 ml) was added N-iodosuccinimide (1360 mg, 6.04 mmol) at 20° C. The reaction mixture was stirred at 20° C. for 12 hours. The reaction mixture was poured into sodium sulfite aqueous solution and extracted with ethyl acetate. The combined organic layers were washed with brine, dried over anhydrous sodium sulfate, filtered and concentrated in vacuo to give tert-butyl 2-(3-chloro-4-methoxyphenyl)-3-iodo-6,7-dihydropyrazolo[1,5-a]pyrazine-5(4H)-carboxylate (1.20 g, 2.45 mmol, 81% yield) as a black gum.

LC-MS (Method 11): Rₜ=1.064 min; MS (ESIpos): m/z=490.0 [M+H]⁺

### Intermediate 57

### tert-butyl 2-(3-chloro-4-methoxyphenyl)-3-(pyridin-4-yl)-6,7-dihydropyrazolo[1,5-a]pyrazine-5(4H)-carboxylate

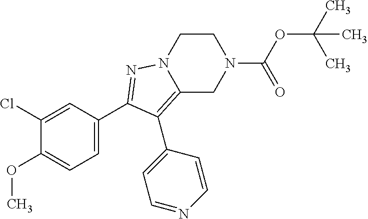 [SMILES: COc1ccc(-c2nn3c(c2-c2ccncc2)CN(C(=O)OC(C)(C)C)CC3)cc1Cl]

To a mixture of tert-butyl 2-(3-chloro-4-methoxyphenyl)-3-iodo-6,7-dihydropyrazolo[1,5-a]pyrazine-5(4H)-carboxylate (1.10 g, 2.24 mmol, Intermediate 56) and pyridin-4-ylboronic acid (304 mg, 2.47 mmol) in 1,4-dioxane (66 ml) and water (22 ml) were added dichloro[1,1-bis(diphenylphosphino) ferrocene]palladium (II) (164 mg, 0.224 mmol) and sodium carbonate (476 mg, 4.47 mmol) at 20° C. The resulting mixture was stirred at 80° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a crude product. The crude product was purified by column chromatography on silica gel (petroleum ether:ethyl acetate=1:1) to give tert-butyl 2-(3-chloro-4-methoxyphenyl)-3-(pyridin-4-yl)-6,7-dihydropyrazolo[1,5-a]pyrazine-5(4H)-carboxylate (780 mg, 1.76 mmol, 78% yield) as a brown solid.

LC-MS (Method 11): Rₜ=0.857 min; MS (ESIpos): m/z=429.1 [M+H]⁺

### Intermediate 58

### 2-(3-chloro-4-methoxyphenyl)-3-(pyridin-4-yl)-4,5,6,7-tetrahydropyrazolo[1,5-a]pyrazine hydrochloride (1:1)

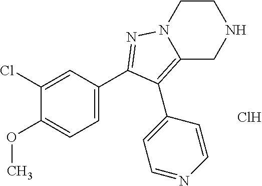 [SMILES: COc1ccc(-c2nn3c(c2-c2ccncc2)CNCC3)cc1Cl ; Cl]

To a solution of tert-butyl 2-(3-chloro-4-methoxyphenyl)-3-(pyridin-4-yl)-6,7-dihydropyrazolo[1,5-a]pyrazine-5(4H)-carboxylate (780 mg, 1.76 mmol, Intermediate 57) in ethyl acetate (55 ml) was added hydrochloric acid (2.2 ml, 4 M in ethyl acetate) at 20° C. The resulting mixture was stirred at 20° C. for 1 hour. The reaction mixture was filtered and the solid cake was collected to give 2-(3-chloro-4-methoxyphenyl)-3-(pyridin-4-yl)-4,5,6,7-tetrahydropyrazolo[1,5-a]pyrazine hydrochloride (1:1) (650 mg, 1.72 mmol, 97% yield) as a yellow solid.

LC-MS (Method 11): Rₜ=0.840 min; MS (ESIpos): m/z=341.1 [M+H]⁺

### Intermediate 59

### 2-(2-methoxyphenyl)-6,7-dihydropyrazolo[1,5-a]pyrazin-4 (5H)-one

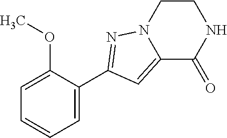 [SMILES: COc1ccccc1-c1cc2n(n1)CCNC2=O]

To a solution of 2-bromo-6,7-dihydropyrazolo[1,5-a]pyrazin-4 (5H)-one (600 mg, 2.78 mmol, Intermediate 40) and (2-methoxyphenyl) boronic acid (464 mg, 3.06 mmol) in 1,4-dioxane (10 ml) and water (2 ml) were added sodium carbonate (589 mg, 5.55 mmol) and (1,1′-bis(diphenylphosphino) ferrocene) palladium (II) dichloride (203 mg, 278 μmol) at 20° C. The mixture was degassed with nitrogen and then stirred at 90° C. for 16 hours. The reaction mixture was poured into water and extracted with ethyl acetate. Combined extracts were dried over anhydrous sodium sulfate and concentrated under vacuum to give 2-(2-methoxyphenyl)-6,7-dihydropyrazolo[1,5-a]pyrazin-4 (5H)-one (600 mg, 2.47 mmol, 89% yield) as a brown solid.

LC-MS (Method 16): Rₜ=0.680 min; MS (ESIpos): m/z=244.1 [M+H]⁺.

### Intermediate 60

### 2-(2-methoxyphenyl)-4,5,6,7-tetrahydropyrazolo[1,5-a]pyrazine

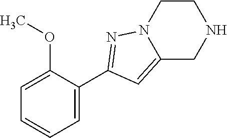 [SMILES: COc1ccccc1-c1cc2n(n1)CCNC2]

To a solution of 2-(2-methoxyphenyl)-6,7-dihydropyrazolo[1,5-a]pyrazin-4 (5H)-one (600 mg, 2.47 mmol, Intermediate 59) in tetrahydrofuran (30 ml) was added dropwise borane tetrahydrofuran complex (15 ml, 1.0 M, 15 mmol) via syringe at 0° C. under a nitrogen atmosphere. The mixture was then stirred at 65° C. for 12 hours. The reaction was quenched with methanol and the resulting mixture was concentrated under vacuum to give 2-(2-methoxyphenyl)-4,5,6,7-tetrahydropyrazolo[1,5-a]pyrazine (565 mg, 2.46 mmol, 100% yield) as a black gum.

LC-MS (Method 11): Rₜ=0.736 min; MS (ESIpos): m/z=230.2 [M+H]⁺

### Intermediate 61

### tert-butyl 2-(2-methoxyphenyl)-6,7-dihydropyrazolo[1,5-a]pyrazine-5(4H)-carboxylate

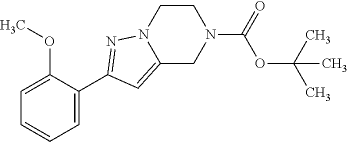 [SMILES: COc1ccccc1-c1cc2n(n1)CCN(C(=O)OC(C)(C)C)C2]

To a solution of 2-(2-methoxyphenyl)-4,5,6,7-tetrahydropyrazolo[1,5-a]pyrazine (565 mg, 2.46 mmol, Intermediate 60) in dichloromethane (20 ml) were added di-tert-butyl dicarbonate (1.08 g, 4.93 mmol), triethylamine (1.0 ml, 7.4 mmol) and 4-(Dimethylamino)pyridine (30.1 mg, 246 μmol) at 20° C. The resulting mixture was stirred at 20° C. for 2 hours. The reaction mixture was concentrated under vacuum to give a crude product and the crude product product was purified by silica gel column chromatography (petroleum ether:ethyl aceate=1:0 to 3:1) to give tert-butyl 2-(2-methoxyphenyl)-6,7-dihydropyrazolo[1,5-a]pyrazine-5(4H)-carboxylate (700 mg, 2.13 mmol, 86% yield) as a yellow oil.

LC-MS (Method 11): Rₜ=0.846 min; MS (ESIpos): m/z=330.2 [M+H]⁺

### Intermediate 62

### tert-butyl 3-iodo-2-(2-methoxyphenyl)-6,7-dihydropyrazolo[1,5-a]pyrazine-5(4H)-carboxylate

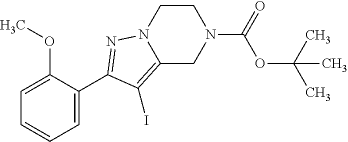 [SMILES: COc1ccccc1-c1nn2c(c1I)CN(C(=O)OC(C)(C)C)CC2]

To a solution of tert-butyl 2-(2-methoxyphenyl)-6,7-dihydropyrazolo[1,5-a]pyrazine-5(4H)-carboxylate (700 mg, 2.13 mmol, Intermediate 61) in dichloromethane (10 ml) and methanol (2.5 ml) was added 1-iodopyrrolidine-2,5-dione (956 mg, 4.25 mmol) in portions at 20° C. and the reaction mixture was stirred at 43° C. for 16 hours. The reaction was quenched with saturated sodium sulfite aqueous solution and the resulting mixture was extracted with ethyl acetate. Combined extracts were dried over anhydrous sodium sulfate and concentrated under vacuum to give tert-butyl 3-iodo-2-(2-methoxyphenyl)-6,7-dihydropyrazolo[1,5-a]pyrazine-5(4H)-carboxylate (800 mg, 1.76 mmol, 83% yield) as a yellow oil.

LC-MS (Method 11): Rₜ=1.021 min; MS (ESIpos): m/z=456.1 [M+H]⁺

### Intermediate 63

### tert-butyl 2-(2-methoxyphenyl)-3-(pyridin-4-yl)-6,7-dihydropyrazolo[1,5-a]pyrazine-5(4H)-carboxylate

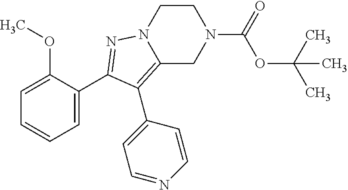 [SMILES: COc1ccccc1-c1nn2c(c1-c1ccncc1)CN(C(=O)OC(C)(C)C)CC2]

To a solution of tert-butyl 3-iodo-2-(2-methoxyphenyl)-6,7-dihydropyrazolo[1,5-a]pyrazine-5(4H)-carboxylate (780 mg, 1.71 mmol, Intermediate 62) and pyridin-4-ylboronic acid (421 mg, 3.43 mmol) in 1,4-dioxane (8 ml) and water (2 ml) were added sodium carbonate (363 mg, 3.43 mmol) and (1,1′-bis(diphenylphosphino) ferrocene) palladium (II) dichloride (125 mg, 171 μmol) at 20° C. The mixture was degassed with nitrogen and stirred at 80° C. for 16 hours. The reaction mixture was concentrated under vacuum to give a crude product and the crude product was purified by silica gel column chromatography (petroleum ether:ethyl aceate=1:0 to 0:1) to give tert-butyl 2-(2-methoxyphenyl)-3-(pyridin-4-yl)-6,7-dihydropyrazolo[1,5-a]pyrazine-5(4H)-carboxylate (300 mg, 738 μmol, 43% yield) as a yellow oil.

LC-MS (Method 11): Rₜ=0.810 min; MS (ESIpos): m/z=407.3 [M+H]⁺.

### Intermediate 64

### 2-(2-methoxyphenyl)-3-(pyridin-4-yl)-4,5,6,7-tetrahydropyrazolo[1,5-a]pyrazine hydrogen chloride

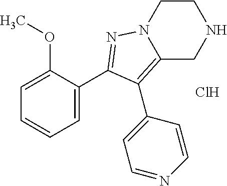 [SMILES: COc1ccccc1-c1nn2c(c1-c1ccncc1)CNCC2 ; Cl]

To a solution of hydrochloric acid (538 mg, 14.8 mmol) in ethyl aceate (10 ml) was added tert-butyl 2-(2-methoxyphenyl)-3-(pyridin-4-yl)-6,7-dihydropyrazolo[1,5-a]pyrazine-5(4H)-carboxylate (300 mg, 738 μmol, Intermediate 63) at 20° C. and the reaction mixture was stirred at 20° C. for 1 hour. The insolubles were collected by filtration to give 2-(2-methoxyphenyl)-3-(pyridin-4-yl)-4,5,6,7-tetrahydropyrazolo[1,5-a]pyrazine hydrogen chloride (240 mg, 700 μmol, 95% yield) as a brown solid.

LC-MS (Method 14): Rₜ=0.126 min; MS (ESIpos): m/z=307.1 [M+H]⁺.

### Intermediate 65

### N-[(3-bromo-1-{2-[(tert-butoxycarbonyl)amino]ethyl}-1H-pyrazol-5-yl)methyl]-N,N-diethylethanaminium methanesulfonate

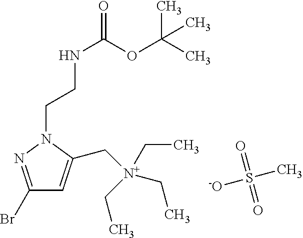

To a solution of tert-butyl {2-[3-bromo-5-(hydroxymethyl)-1H-pyrazol-1-yl]ethyl}carbamate (14.0 g, 43.7 mmol, Intermediate 38) and methanesulfonic anhydride (17.4 g, 99.9 mmol) in dichloromethane (160 ml) was added triethyl amine (14 ml, 99.9 mmol) at 20° C. The resulting mixture was stirred at 20° C. for 16 hours. The reaction mixture was concentrated in vacuo to give N-[(3-bromo-1-{2-[(tert-butoxycarbonyl)amino]ethyl}-1H-pyrazol-5-yl)methyl]-N,N-diethylethanaminium methanesulfonate (24.0 g, 48.1 mmol, 96% yield) as a pale brown oil.

LC-MS (Method 11): Rₜ=0.710 min; MS (ESIpos): m/z=403.2 [M+H]⁺.

### Intermediate 66

### tert-butyl 2-bromo-6,7-dihydropyrazolo[1,5-a]pyrazine-5(4H)-carboxylate

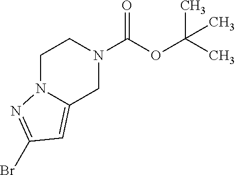 [SMILES: CC(C)(C)OC(=O)N1CCn2nc(Br)cc2C1]

To a mixture of N-[(3-bromo-1-{2-[(tert-butoxycarbonyl)amino]ethyl}-1H-pyrazol-5-yl)methyl]-N,N-diethylethanaminium methanesulfonate (24.0 g, 48.1 mmol, Intermediate 65) in tetrahydrofuran (80 ml) and N,N-dimethylformamide (80 ml) was added a solution of sodium hydride (3.8 g, 60% purity, 96.1 mmol) in tetrahydrofuran (20 ml) at 0° C. The resulting mixture was stirred at 20° C. for 2 hours. The reaction was quenched with water and extracted with ethyl acetate. The combined organic layers was washed with brine, dried over anhydrous sodium sulfate, filtered and concentrated in vacuo to give tert-butyl 2-bromo-6,7-dihydropyrazolo[1,5-a]pyrazine-5(4H)-carboxylate (14 g, 46.3 mmol, 96% yield) as a brown oil.

LC-MS (Method 11): Rₜ=0.910 min; MS (ESIpos): m/z=302.0 [M+H]⁺.

### Intermediate 67

### tert-butyl 2-bromo-3-iodo-6,7-dihydropyrazolo[1,5-a]pyrazine-5(4H)-carboxylate

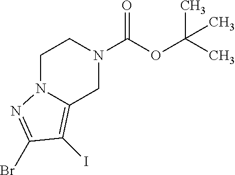 [SMILES: CC(C)(C)OC(=O)N1CCn2nc(Br)c(I)c2C1]

To a solution of tert-butyl 2-bromo-6,7-dihydropyrazolo[1,5-a]pyrazine-5(4H)-carboxylate (14 g, 46.3 mmol, Intermediate 66) in dichloromethane (100 ml) and methanol (50 ml) was added 1-iodopyrrolidine-2,5-dione (15.6 g, 69.5 mmol) at 20° C. The resulting mixture was stirred at 43° C. for 12 hour. The reaction mixture was poured into saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=10:1) to give tert-butyl 2-bromo-3-iodo-6,7-dihydropyrazolo[1,5-a]pyrazine-5(4H)-carboxylate (10.5 g, 24.5 mmol, 53% yield) as a yellow oil.

LC-MS (Method 11): Rₜ=0.684 min; MS (ESIpos): m/z=428.0 [M+H]⁺

¹H-NMR (400 MHz, DMSO-d6) delta [ppm]: 1.090 (0.49), 1.107 (0.47), 1.133 (0.45), 1.155 (0.59), 1.173 (0.97), 1.191 (0.97), 1.219 (0.69), 1.235 (0.62), 1.907 (0.45), 1.987 (0.49), 2.564 (3.35), 2.670 (0.43), 2.751 (0.44), 3.058 (4.69), 3.071 (8.34), 3.085 (5.15), 3.686 (16.00), 3.751 (0.56), 3.914 (4.88), 3.928 (8.30), 3.941 (4.61).

### Intermediate 68

### tert-butyl 2-bromo-3-(pyridin-4-yl)-6,7-dihydropyrazolo[1,5-a]pyrazine-5(4H)-carboxylate

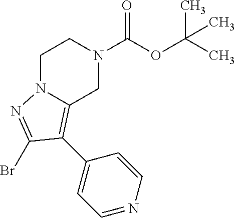 [SMILES: CC(C)(C)OC(=O)N1CCn2nc(Br)c(-c3ccncc3)c2C1]

To a mixture of tert-butyl 2-bromo-3-iodo-6,7-dihydropyrazolo[1,5-a]pyrazine-5(4H)-carboxylate (8.50 g, 19.9 mmol, Intermediate 67) and pyridin-4-ylboronic acid (2.44 g, 19.9 mmol) in 1,4-dioxane (85 ml) and water (26 ml) were added dichloro[1,1-bis(diphenylphosphino) ferrocene]palladium (II) (1.45 g, 1.99 mmol) and sodium carbonate (4.21 g, 39.7 mmol) at 20° C. The mixture was stirred at 80° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 1:1) to give tert-butyl 2-bromo-3-(pyridin-4-yl)-6,7-dihydropyrazolo[1,5-a]pyrazine-5(4H)-carboxylate (5.8 g, 15.3 mmol, 77% yield) as a yellow solid.

LC-MS (Method 11): Rₜ=0.460 min; MS (ESIpos): m/z=379.1 [M+H]⁺.

¹H NMR (400 MHz, DMSO-d6) δ [ppm]=8.81-8.52 (m, 2H), 7.56-7.23 (m, 2H), 4.70 (s, 2H), 4.16 (t, J=5.4 Hz, 2H), 3.86 (t, J=5.4 Hz, 2H), 1.41 (s, 9H).

### Intermediate 69

### tert-butyl 2-(3,4-dichlorophenyl)-3-(pyridin-4-yl)-6,7-dihydropyrazolo[1,5-a]pyrazine-5(4H)-carboxylate

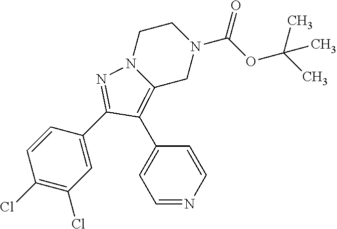 [SMILES: CC(C)(C)OC(=O)N1CCn2nc(-c3ccc(Cl)c(Cl)c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (300 mg, 0.791 mmol, Intermediate 68) and (3,4-dichlorophenyl) boronic acid (196 mg, 1.02 mml) in 1,4-dioxane (8 ml) and water (2 ml) were added sodium carbonate (167 mg, 1.58 mmol) and dichloro[1,1-bis(diphenylphosphino) ferrocene]palladium (II) (57.8 mg, 0.0791 mmol) at 20° C. The mixture was degassed with nitrogen and then stirred at 90° C. for 16 hours. The reaction solution was poured into water and extracted with ethyl acetate. The combined organic layers were washed with brine, dried over anhydrous sodium sulfate, filtered and the filtrate was concentrated in vacuo to give tert-butyl 2-(3,4-dichlorophenyl)-3-(pyridin-4-yl)-6,7-dihydropyrazolo[1,5-a]pyrazine-5(4H)-carboxylate (270 mg, 0.606 mmol, 76% yield) as a yellow solid.

LC-MS (Method 11): Rₜ=0.565 min; MS (ESIpos): m/z=445.1 [M+H]⁺.

### Intermediate 70

### 2-(3,4-dichlorophenyl)-3-(pyridin-4-yl)-4,5,6,7-tetrahydropyrazolo[1,5-a]pyrazine

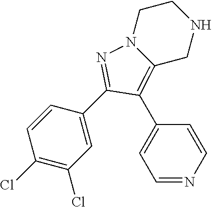 [SMILES: Clc1ccc(-c2nn3c(c2-c2ccncc2)CNCC3)cc1Cl]

To a solution of tert-butyl 2-(3,4-dichlorophenyl)-3-(pyridin-4-yl)-6,7-dihydropyrazolo[1,5-a]pyrazine-5(4H)-carboxylate (272 mg, 0.610 mmol, Intermediate 69) in ethyl acetate (3 ml) was added hydrochloric acid (5 ml, 4 M in ethyl acetate) at 20° C. The mixture was stirred at 20° C. for 1 hour. The reaction mixture was filtered and the solid cake was collected to give 2-(3,4-dichlorophenyl)-3-(pyridin-4-yl)-4,5,6,7-tetrahydropyrazolo[1,5-a]pyrazine (200 mg, 0.579 mmol, 94% yield) as a brown solid.

LC-MS (Method 11): Rₜ=0.416 min; MS (ESIpos): m/z=345.0 [M+H]⁺

### Intermediate 71

### 2-(4-methoxyphenyl)-6,7-dihydropyrazolo[1,5-a]pyrazin-4 (5H)-one

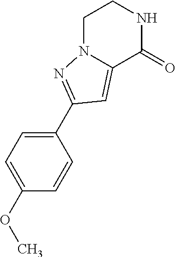 [SMILES: COc1ccc(-c2cc3n(n2)CCNC3=O)cc1]

To a mixture of 2-bromo-6,7-dihydropyrazolo[1,5-a]pyrazin-4 (5H)-one (450 mg, 2.08 mmol, Intermediate 40) and (4-methoxyphenyl) boronic acid (348 mg, 2.29 mmol) in 1,4-dioxane (14 ml) and water 14 ml) were added dichloro[1,1-bis(diphenylphosphino) ferrocene]palladium (II) (152 mg, 0.208 mmol) and sodium carbonate (442 mg, 4.17 mmol) at 20° C. The resulting mixture was stirred at 90° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=0:1) to give 2-(4-methoxyphenyl)-6,7-dihydropyrazolo[1,5-a]pyrazin-4 (5H)-one (390 mg, 1.60 mmol, 77% yield) as a white solid.

LC-MS (Method 12): Rₜ=0.675 min; MS (ESIpos): m/z=244.0 [M+H]⁺.

### Intermediate 72

### 2-(4-methoxyphenyl)-4,5,6,7-tetrahydropyrazolo[1,5-a]pyrazine

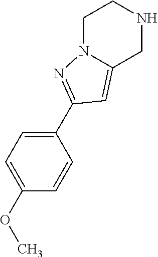 [SMILES: COc1ccc(-c2cc3n(n2)CCNC3)cc1]

To a solution of 2-(4-methoxyphenyl)-6,7-dihydropyrazolo[1,5-a]pyrazin-4 (5H)-one (390 mg, 1.60 mmol, Intermediate 71) in tetrahydrofuran (10 ml) was added borane dimethyl sulfide complex (0.5 ml, 10 M in tetrahydrofuran) at 20° C. and the resulting solution was stirred at 60° C. for 16 hours. The reaction was quenched with methanol and the resulting solution was concentrated in vacuo to give 2-(4-methoxyphenyl)-4,5,6,7-tetrahydropyrazolo[1,5-a]pyrazine (350 mg, 1.53 mmol, 95% yield) as a white solid.

LC-MS (Method 12): Rₜ=0.723 min; MS (ESIpos): m/z=230.0 [M+H]⁺

### Intermediate 73

### tert-butyl 2-(4-methoxyphenyl)-6,7-dihydropyrazolo[1,5-a]pyrazine-5(4H)-carboxylate

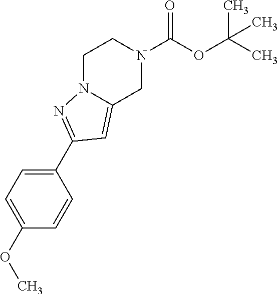 [SMILES: COc1ccc(-c2cc3n(n2)CCN(C(=O)OC(C)(C)C)C3)cc1]

To a solution of 2-(4-methoxyphenyl)-4,5,6,7-tetrahydropyrazolo[1,5-a]pyrazine (350 mg, 1.53 mmol, Intermediate 72) in dichloromethane (5 ml) were added trimethylamine (0.4 ml, 3.05 mmol), 4-(dimethylamino)pyridine (18.6 mg, 0.153 mmol) and di-tert-butyl dicarbonate (0.7 ml, 3.05 mmol) at 20° C. The resulting mixture was stirred at 20° C. for 4 hours. The reaction mixture was poured into water and extracted with ethyl acetate. The combined organic layers were washed with brine, dried over anhydrous sodium sulfate, filtered and concentrated in vacuo to give tert-butyl 2-(4-methoxyphenyl)-6,7-dihydropyrazolo[1,5-a]pyrazine-5(4H)-carboxylate (450 mg, 1.37 mmol, 89% yield) as a pale yellow oil.

LC-MS (Method 12): Rₜ=0.947 min; MS (ESIpos): m/z=330.3 [M+H]⁺.

### Intermediate 74

### tert-butyl 3-iodo-2-(4-methoxyphenyl)-6,7-dihydropyrazolo[1,5-a]pyrazine-5(4H)-carboxylate

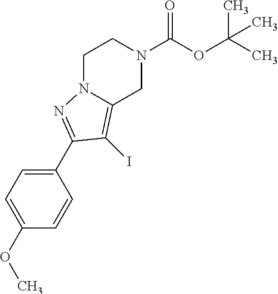 [SMILES: COc1ccc(-c2nn3c(c2I)CN(C(=O)OC(C)(C)C)CC3)cc1]

To a solution of tert-butyl 2-(4-methoxyphenyl)-6,7-dihydropyrazolo[1,5-a]pyrazine-5(4H)-carboxylate (450 mg, 1.37 mmol, Intermediate 73) in dichloromethane (5 ml) was added N-iodosuccinimide (922 mg, 4.10 mmol) at 20° C. The reaction mixture was stirred at 20° C. for 2 hours. The reaction mixture was poured into sodium sulfite aqueous solution and extracted with ethyl acetate. The combined organic layers were washed with brine, dried over anhydrous sodium sulfate, filtered and concentrated in vacuo to give tert-butyl 3-iodo-2-(4-methoxyphenyl)-6,7-dihydropyrazolo[1,5-a]pyrazine-5(4H)-carboxylate (450 mg, 0.988 mmol, 72% yield) as a pale yellow solid.

LC-MS (Method 12): Rₜ=1.019 min; MS (ESIpos): m/z=456.2 [M+H]⁺

### Intermediate 75

### tert-butyl 2-(4-methoxyphenyl)-3-(pyridin-4-yl)-6,7-dihydropyrazolo[1,5-a]pyrazine-5(4H)-carboxylate

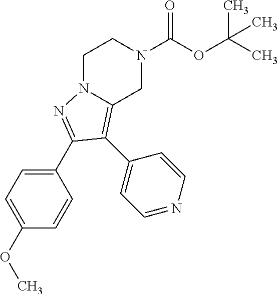 [SMILES: COc1ccc(-c2nn3c(c2-c2ccncc2)CN(C(=O)OC(C)(C)C)CC3)cc1]

To a mixture of tert-butyl 3-iodo-2-(4-methoxyphenyl)-6,7-dihydropyrazolo[1,5-a]pyrazine-5(4H)-carboxylate (350 mg, 0.769 mmol, Intermediate 74) and pyridin-4-ylboronic acid (113 mg, 0.922 mmol) in 1,4-dioxane (14 ml) and water (4 ml) were added dichloro[1,1-bis(diphenylphosphino) ferrocene]palladium (II) (56.2 mg, 0.0769 mmol) and sodium carbonate (163 mg, 1.54 mmol) at 20° C. The resulting mixture was stirred at 80° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a crude product.

The crude product was purified by column chromatography on silica gel (petroleum ether:ethyl acetate=2:1 to 0:1) to give tert-butyl 2-(4-methoxyphenyl)-3-(pyridin-4-yl)-6,7-dihydropyrazolo[1,5-a]pyrazine-5(4H)-carboxylate (160 mg, 0.394 mmol, 51% yield) as a brown solid.

LC-MS (Method 12): Rₜ=0.717 min; MS (ESIpos): m/z=407.3 [M+H]⁺

### Intermediate 76

### 2-(4-methoxyphenyl)-3-(pyridin-4-yl)-4,5,6,7-tetrahydropyrazolo[1,5-a]pyrazine hydrochloride (1:1)

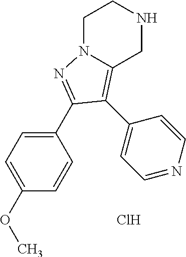 [SMILES: COc1ccc(-c2nn3c(c2-c2ccncc2)CNCC3)cc1 ; Cl]

To a solution of tert-butyl 2-(4-methoxyphenyl)-3-(pyridin-4-yl)-6,7-dihydropyrazolo[1,5-a]pyrazine-5(4H)-carboxylate (160 mg, 0.394 mmol, Intermediate 75) in ethyl acetate (9 ml) was added hydrochloric acid (0.5 ml, 4 M in ethyl acetate) at 20° C. The resulting mixture was stirred at 20° C. for 2 hours. The reaction mixture was filtered and the solid cake was collected to give 2-(4-methoxyphenyl)-3-(pyridin-4-yl)-4,5,6,7-tetrahydropyrazolo[1,5-a]pyrazine hydrochloride (1:1) (120 mg, 0.350 mmol, 89% yield) as a yellow solid.

### Intermediate 77

### 2-(3-fluoro-4-methoxyphenyl)-6,7-dihydropyrazolo[1,5-a]pyrazin-4 (5H)-one

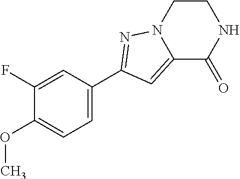 [SMILES: COc1ccc(-c2cc3n(n2)CCNC3=O)cc1F]

To a mixture of 2-bromo-6,7-dihydropyrazolo[1,5-a]pyrazin-4 (5H)-one (600 mg, 2.78 mmol, Intermediate 40) and (3-fluoro-4-methoxyphenyl) boronic acid (520 mg, 3.06 mmol) in 1,4-dioxane (6 ml) and water (2 ml) were added dichloro[1,1-bis(diphenylphosphino) ferrocene]palladium (II) (203 mg, 0.278 mmol) and sodium carbonate (589 mg, 5.55 mmol). The resulting mixture was stirred at 90° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=0:1) to give 2-(3-fluoro-4-methoxyphenyl)-6,7-dihydropyrazolo[1,5-a]pyrazin-4 (5H)-one (120 mg, 0.459 mmol, 17% yield) as a white solid.

LC-MS (Method 11): Rₜ=0.801 min; MS (ESIpos): m/z=262.1 [M+H]⁺.

### Intermediate 78

### 2-(3-fluoro-4-methoxyphenyl)-4,5,6,7-tetrahydropyrazolo[1,5-a]pyrazine

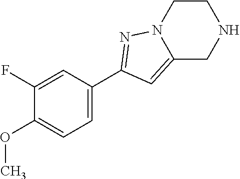 [SMILES: COc1ccc(-c2cc3n(n2)CCNC3)cc1F]

To a solution of 2-(3-fluoro-4-methoxyphenyl)-6,7-dihydropyrazolo[1,5-a]pyrazin-4 (5H)-one (120 mg, 0.459 mmol, Intermediate 77) in tetrahydrofuran (5 ml) was added borane tetrahydrofuran complex (1.4 ml, 1 M in tetrahydrofuran) at 20° C. The mixture was stirred at 65° C. for 12 hours. The reaction was quenched with methanol and the resulting mixture was concentrated in vacuo to give 2-(3-fluoro-4-methoxyphenyl)-4,5,6,7-tetrahydropyrazolo[1,5-a]pyrazine (110 mg, 0.445 mmol, 97% yield) as a black oil.

LC-MS (Method 11): Rₜ=0.837 min; MS (ESIpos): m/z=248.1 [M+H]⁺.

### Intermediate 79

### tert-butyl 2-(3-fluoro-4-methoxyphenyl)-6,7-dihydropyrazolo[1,5-a]pyrazine-5(4H)-carboxylate

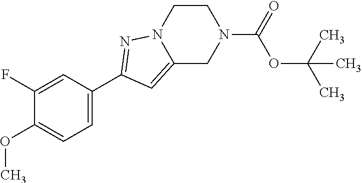 [SMILES: COc1ccc(-c2cc3n(n2)CCN(C(=O)OC(C)(C)C)C3)cc1F]

To a solution of 2-(3-fluoro-4-methoxyphenyl)-4,5,6,7-tetrahydropyrazolo[1,5-a]pyrazine (110 mg, 0.445 mmol, Intermediate 78) and di-tert-butyl dicarbonate (194 mg, 0.900 mmol) in dichloromethane (5 ml) were added trimethylamine (0.2 ml) and 4-(dimethylamino)pyridine (5.44 mg, 0.0445 mmol) at 20° C. The resulting mixture was stirred at 20° C. for 1 hour. The reaction mixture was poured into water and extracted with ethyl acetate. The combined organic layers were washed with brine, dried over anhydrous sodium sulfate, filtered and concentrated in vacuo to give tert-butyl 2-(3-fluoro-4-methoxyphenyl)-6,7-dihydropyrazolo[1,5-a]pyrazine-5(4H)-carboxylate (130 mg, 0.374 mmol, 84% yield) as a pale brown solid.

LC-MS (Method 11): Rₜ=0.878 min; MS (ESIpos): m/z=348.2 [M+H]⁺

### Intermediate 80

### tert-butyl 2-(3-fluoro-4-methoxyphenyl)-3-iodo-6,7-dihydropyrazolo[1,5-a]pyrazine-5(4H)-carboxylate

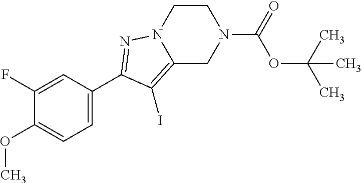 [SMILES: COc1ccc(-c2nn3c(c2I)CN(C(=O)OC(C)(C)C)CC3)cc1F]

To a solution of tert-butyl 2-(3-fluoro-4-methoxyphenyl)-6,7-dihydropyrazolo[1,5-a]pyrazine-5(4H)-carboxylate (130 mg, 0.374 mmol, Intermediate 79) in dichloromethane (10 ml) and methanol (3 ml) was added N-iodosuccinimide (92.0 mg, 0.412 mmol) at 20° C. The mixture was stirred at 43° C. for 12 hours. The reaction mixture was poured into saturated sodium sulfite aqueous solution and extracted with ethyl acetate. The combined organic layers were washed with brine, dried over anhydrous sodium sulfate, filtered and concentrated in vacuo to give tert-butyl 2-(3-fluoro-4-methoxyphenyl)-3-iodo-6,7-dihydropyrazolo[1,5-a]pyrazine-5(4H)-carboxylate (150 mg, 0.317 mmol, 85% yield) as a brown solid.

LC-MS (Method 11): Rₜ=1.034 min; MS (ESIpos): m/z=474.2 [M+H]⁺

### Intermediate 81

### tert-butyl 2-(3-fluoro-4-methoxyphenyl)-3-(pyridin-4-yl)-6,7-dihydropyrazolo[1,5-a]pyrazine-5(4H)-carboxylate

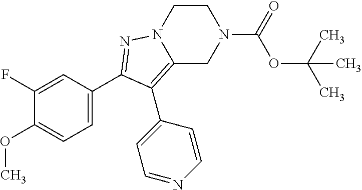 [SMILES: COc1ccc(-c2nn3c(c2-c2ccncc2)CN(C(=O)OC(C)(C)C)CC3)cc1F]

To a solution of tert-butyl 2-bromo-3-(pyridin-4-yl)-6,7-dihydropyrazolo[1,5-a]pyrazine-5(4H)-carboxylate (150 mg, 0.317 mmol, Intermediate 80) and (3-fluoro-4-methoxyphenyl) boronic acid (46.7 mg, 0.380 mmol) in 1,4-dioxane (6 ml) and water (2 ml) were added sodium carbonate (67.2 mg, 0.634 mmol) and dichloro[1,1-bis(diphenylphosphino) ferrocene]palladium (II) (23.2 mg, 0.0317 mmol) at 20° C. The mixture was degassed with nitrogen and then stirred at 90° C. for 16 hours. The reaction mixture was poured into water, extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=1:1) to give tert-butyl 2-(3-fluoro-4-methoxyphenyl)-3-(pyridin-4-yl)-6,7-dihydropyrazolo[1,5-a]pyrazine-5(4H)-carboxylate (100 mg, 0.236 mmol, 74% yield) as a yellow solid.

LC-MS (Method 11): Rₜ=0.819 min; MS (ESIpos): m/z=425.3 [M+H]⁺

### Intermediate 82

### 2-(3-fluoro-4-methoxyphenyl)-3-(pyridin-4-yl)-4,5,6,7-tetrahydropyrazolo[1,5-a]pyrazine hydrochloride (1:1)

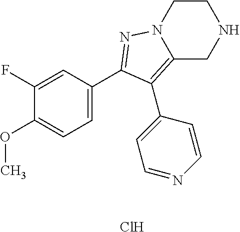 [SMILES: COc1ccc(-c2nn3c(c2-c2ccncc2)CNCC3)cc1F ; Cl]

To a solution of tert-butyl 2-(3-fluoro-4-methoxyphenyl)-3-(pyridin-4-yl)-6,7-dihydropyrazolo[1,5-a]pyrazine-5(4H)-carboxylate (100 mg, 0.236 mmol, Intermediate 81) in ethyl acetate (5 ml) was added hydrochloric acid (0.3 ml, 4 M in ethyl acetate) at 20° C. The mixture was stirred at 20° C. for 1 hour. The reaction mixture was filtered and the solid cake was collected to give 2-(3-fluoro-4-methoxyphenyl)-3-(pyridin-4-yl)-4,5,6,7-tetrahydropyrazolo[1,5-a]pyrazine hydrochloride (1:1) (60 mg, 0.166 mmol, 71% yield) as a yellow solid.

### Intermediate 83

### tert-butyl 2-(4-cyanophenyl)-3-(pyridin-4-yl)-6,7-dihydropyrazolo[1,5-a]pyrazine-5(4H)-carboxylate

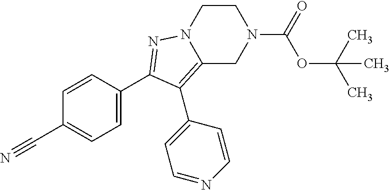 [SMILES: CC(C)(C)OC(=O)N1CCn2nc(-c3ccc(C#N)cc3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.00 mmol, Intermediate 68) in 1,4-dioxane (4.0 ml) and water (1 ml) were added (4-cyanophenyl) boronic acid (177 mg, 1.20 mmol), sodium carbonate (212 mg, 2.00 mmol) and dichloro[1,1-bis(diphenylphosphino) ferrocene]palladium (II) (73 mg, 0.10 mmol) at 25° C. The reaction mixture was stirred at 90° C. for 18 hours under nitrogen atmosphere. The mixture was filtered through a pad of celite and the filtrate was concentrated in vacuo to give a residue. The residue was purified by column chromatography on silica gel (100~200 mesh, petroleum ether:ethyl acetate=5:1) to give tert-butyl 2-(4-cyanophenyl)-3-(pyridin-4-yl)-6,7-dihydropyrazolo[1,5-a]pyrazine-5(4H)-carboxylate (310 mg, 87% purity, 54% yield) as a yellow solid.

LC-MS (Method 13): Rₜ=0.524 min; MS (ESIpos): m/z=402.3 [M+H]⁺.

### Intermediate 84

### 4-[3-(pyridin-4-yl)-4,5,6,7-tetrahydropyrazolo[1,5-a]pyrazin-2-yl]benzonitrile-hydrogen chloride (1/1)

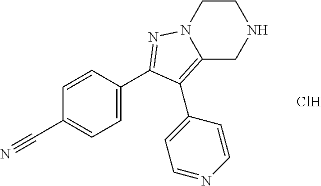 [SMILES: N#Cc1ccc(-c2nn3c(c2-c2ccncc2)CNCC3)cc1 ; Cl]

The reaction mixture of tert-butyl 2-(4-cyanophenyl)-3-(pyridin-4-yl)-6,7-dihydropyrazolo[1,5-a]pyrazine-5(4H)-carboxylate (350 mg, 845 μmol, Intermediate 83) in ethyl acetate (10 ml) was added hydrochloric acid (5 ml, 4 M in ethyl acetate) at 25° C. The mixture was stirred at 25° C. for 2 hours. The reaction mixture was concentrated in vacuo to give 2-ethyl-4-[3-(pyridin-4-yl)-4,5,6,7-tetrahydropyrazolo[1,5-a]pyrazin-2-yl]benzonitrile hydrochloride (1:1) (380 mg, 78% purity, 96% yield) as a yellow solid.

LC-MS (Method 13): Rₜ=0.246 min; MS (ESIpos): m/z=302.2 [M+H]⁺

### Intermediate 85

### 2-[4-(trifluoromethyl)phenyl]-6,7-dihydropyrazolo[1,5-a]pyrazin-4 (5H)-one

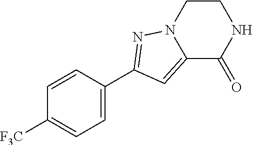 [SMILES: O=C1NCCn2nc(-c3ccc(C(F)(F)F)cc3)cc21]

To a mixture of 2-bromo-6,7-dihydropyrazolo[1,5-a]pyrazin-4 (5H)-one (600 mg, 2.78 mmol, Intermediate 40) and [4-(trifluoromethyl)phenyl]boronic acid (580 mg, 3.06 mmol) in 1,4-dioxane (6 ml) and water (2 ml) were added dichloro[1,1-bis(diphenylphosphino) ferrocene]palladium (II) (203 mg, 0.278 mmol) and sodium carbonate (589 mg, 5.55 mmol) at 20° C. The resulting mixture was stirred at 90° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=0:1) to give 2-[4-(trifluoromethyl)phenyl]-6,7-dihydropyrazolo[1,5-a]pyrazin-4 (5H)-one (220 mg, 0.782 mmol, 28% yield) as a white solid.

LC-MS (Method 11): Rₜ=0.866 min; MS (ESIpos): m/z=282.1 [M+H]⁺

### Intermediate 86

### 2-[4-(trifluoromethyl)phenyl]-4,5,6,7-tetrahydropyrazolo[1,5-a]pyrazine

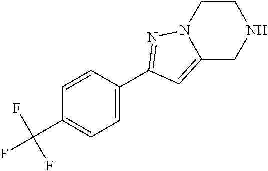 [SMILES: FC(F)(F)c1ccc(-c2cc3n(n2)CCNC3)cc1]

To a solution of 2-[4-(trifluoromethyl)phenyl]-6,7-dihydropyrazolo[1,5-a]pyrazin-4 (5H)-one (220 mg, 0.782 mmol, Intermediate 85) in tetrahydrofuran (3 ml) was added borane tetrahydrofuran complex (2 ml, 1 M in tetrahydrofuran) at 20° C. The mixture was stirred at 65° C. for 12 hours. The reaction was quenched with methanol and the resulting mixture was concentrated in vacuo to give 2-[4-(trifluoromethyl)phenyl]-4,5,6,7-tetrahydropyrazolo[1,5-a]pyrazine (200 mg, 0.748 mmol, 96% yield) as a black oil.

LC-MS (Method 11): Rₜ=0.906 min; MS (ESIpos): m/z=268.1 [M+H]⁺.

### Intermediate 87

### tert-butyl 2-[4-(trifluoromethyl)phenyl]-6,7-dihydropyrazolo[1,5-a]pyrazine-5(4H)-carboxylate

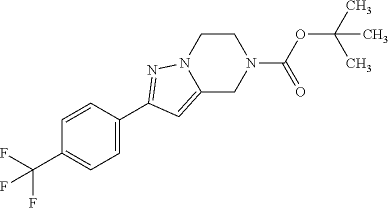 [SMILES: CC(C)(C)OC(=O)N1CCn2nc(-c3ccc(C(F)(F)F)cc3)cc2C1]

To a solution of 2-[4-(trifluoromethyl)phenyl]-4,5,6,7-tetrahydropyrazolo[1,5-a]pyrazine (200 mg, 0.748 mmol, Intermediate 86) and di-tert-butyl dicarbonate (0.344 ml, 1.50 mmol) in dichloromethane (5 ml) were added triethylamine (0.2 ml, 1.50 mmol) and 4-(dimethylamino)pyridine (9.14 mg, 0.0748 mmol) at 20° C. The resulting mixture was stirred at 20° C. for 1 hour. The reaction mixture was poured into water and extracted with ethyl acetate. The combined organic layers were washed with brine, dried over anhydrous sodium sulfate, filtered and concentrated in vacuo to give tert-butyl 2-[4-(trifluoromethyl)phenyl]-6,7-dihydropyrazolo[1,5-a]pyrazine-5(4H)-carboxylate (250 mg, 0.681 mmol, 91% yield) as a pale brown solid.

LC-MS (Method 11): Rₜ=0.936 min; MS (ESIpos): m/z=368.2 [M+H]⁺.

### Intermediate 88

### tert-butyl 3-iodo-2-[4-(trifluoromethyl)phenyl]-6,7-dihydropyrazolo[1,5-a]pyrazine-5(4H)-carboxylate

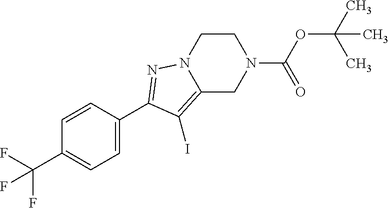 [SMILES: CC(C)(C)OC(=O)N1CCn2nc(-c3ccc(C(F)(F)F)cc3)c(I)c2C1]

To a solution of tert-butyl 2-[4-(trifluoromethyl)phenyl]-6,7-dihydropyrazolo[1,5-a]pyrazine-5(4H)-carboxylate (250 mg, 0.681 mmol, Intermediate 87) in dichloromethane (5 ml) and methanol (5 ml) was added N-iodosuccinimide (322 mg, 1.43 mmol) at 20° C. The mixture was stirred at 43° C. for 12 hours. The reaction mixture was poured into saturated sodium sulfite aqueous solution and extracted with ethyl acetate. The combined organic layers were washed with brine, dried over anhydrous sodium sulfate, filtered and concentrated in vacuo to give tert-butyl 3-iodo-2-[4-(trifluoromethyl)phenyl]-6,7-dihydropyrazolo[1,5-a]pyrazine-5(4H)-carboxylate (300 mg, 0.608 mmol, 89% yield) as a brown solid.

LC-MS (Method 11): Rₜ=1.105 min; MS (ESIpos): m/z=494.2 [M+H]⁺

### Intermediate 89

### tert-butyl 3-(pyridin-4-yl)-2-[4-(trifluoromethyl)phenyl]-6,7-dihydropyrazolo[1,5-a]pyrazine-5(4H)-carboxylate

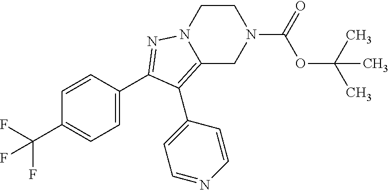 [SMILES: CC(C)(C)OC(=O)N1CCn2nc(-c3ccc(C(F)(F)F)cc3)c(-c3ccncc3)c2C1]

To a solution of tert-butyl 3-iodo-2-[4-(trifluoromethyl)phenyl]-6,7-dihydropyrazolo[1,5-a]pyrazine-5(4H)-carboxylate (300 mg, 0.608 mmol, Intermediate 88) and pyridin-4-ylboronic acid (89.7 mg, 0.730 mmol) in 1,4-dioxane (6 ml) and water (3 ml) were added dichloro[1,1-bis(diphenylphosphino) ferrocene]palladium (II) (44.5 mg, 0.0608 mmol) and sodium carbonate (129 mg, 1.22 mmol) at 20° C. The resulting mixture was stirred at 90° C. for 12 hours under nitrogen atmosphere. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=3:1) to give tert-butyl 3-(pyridin-4-yl)-2-[4-(trifluoromethyl)phenyl]-6,7-dihydropyrazolo[1,5-a]pyrazine-5(4H)-carboxylate (150 mg, 0.337 mmol, 55% yield) as a yellow gum.

LC-MS (Method 11): Rₜ=0.848 min; MS (ESIpos): m/z=445.3 [M+H]⁺

### Intermediate 90

### 3-(pyridin-4-yl)-2-[4-(trifluoromethyl)phenyl]-4,5,6,7-tetrahydropyrazolo[1,5-a]pyrazine hydrochloride (1:1)

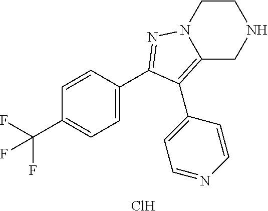 [SMILES: FC(F)(F)c1ccc(-c2nn3c(c2-c2ccncc2)CNCC3)cc1 ; Cl]

To a solution of tert-butyl 3-(pyridin-4-yl)-2-[4-(trifluoromethyl)phenyl]-6,7-dihydropyrazolo[1,5-a]pyrazine-5(4H)-carboxylate (150 mg, 0.337 mmol, Intermediate 89) in ethyl acetate (5 ml) was added hydrochloric acid (0.4 ml, 4 M in ethyl acetate) at 20° C. The mixture was stirred at 20° C. for 1 hour. The reaction mixture was concentrated in vacuo to give 3-(pyridin-4-yl)-2-[4-(trifluoromethyl)phenyl]-4,5,6,7-tetrahydropyrazolo[1,5-a]pyrazine hydrochloride (1:1) (100 mg, 0.263 mmol, 78% yield) as a gray solid.

### Intermediate 91

### tert-butyl 2-[4-chloro-2-(hydroxymethyl)phenyl]-3-(pyridin-4-yl)-6,7-dihydropyrazolo[1,5-a]pyrazine-5(4H)-carboxylate

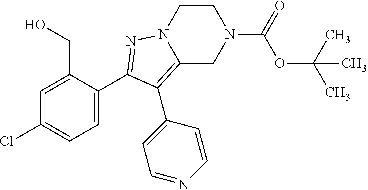 [SMILES: CC(C)(C)OC(=O)N1CCn2nc(-c3ccc(Cl)cc3CO)c(-c3ccncc3)c2C1]

To a solution of tert-butyl 2-bromo-3-(pyridin-4-yl)-6,7-dihydropyrazolo[1,5-a]pyrazine-5(4H)-carboxylate (450 mg, 1.13 mmol, Intermediate 68) in 1,4-dioxane (5.0 ml) and water (1.0 ml) were added [4-chloro-2-(hydroxymethyl)phenyl]boronic acid (252 mg, 1.35 mmol), sodium carbonate (239 mg, 2.25 mmol) and dichloro[1,1-bis(diphenylphosphino) ferrocene]palladium (II) (82.5 mg, 113 μmol) at 25° C. The reaction mixture was stirred at 90° C. for 18 hours under nitrogen atmosphere. The mixture was filtered through a pad of celite and the filtrate was concentrated in vacuo to give a residue. The residue was purified by column chromatography on silica gel (100~200 mesh, petroleum ether:ethyl acetate=5:1) to give tert-butyl 2-[4-chloro-2-(hydroxymethyl)phenyl]-3-(pyridin-4-yl)-6,7-dihydropyrazolo[1,5-a]pyrazine-5(4H)-carboxylate (500 mg, 92% purity, 92% yield) as a yellow solid.

LC-MS (Method 11): Rₜ=0.469 min; MS (ESIpos): m/z=441.1 [M+H]⁺

### Intermediate 92

### {5-chloro-2-[3-(pyridin-4-yl)-4,5,6,7-tetrahydropyrazolo[1,5-a]pyrazin-2-yl]phenyl}methanol

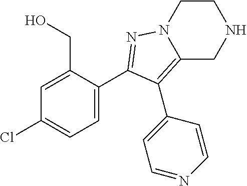 [SMILES: OCc1cc(Cl)ccc1-c1nn2c(c1-c1ccncc1)CNCC2]

To a solution of tert-butyl 2-[4-chloro-2-(hydroxymethyl)phenyl]-3-(pyridin-4-yl)-6,7-dihydropyrazolo[1,5-a]pyrazine-5(4H)-carboxylate (460 mg, 991 μmol, Intermediate 91) in water (5) and dioxane (5 ml) was added sulfuric acid (1 ml) at 20° C. The mixture was stirred at 90° C. for 18 hours. pH of the reaction mixture was adjusted with saturated sodium carbonate aqueous solution to pH=7-8, then the residue was extracted with ethyl acetate. The combined organic phase was washed with brine, dried over anhydrous sodium sulfate, filtered and concentrated in vacuo to give {5-chloro-2-[3-(pyridin-4-yl)-4,5,6,7-tetrahydropyrazolo[1,5-a]pyrazin-2-yl]phenyl}methanol (300 mg, 89% purity, 79% yield) as a pale yellow solid.

LC-MS (Method 11): Rₜ=0.209 min; MS (ESIpos): m/z=341.1 [M+H]⁺

### Intermediate 93

### tert-butyl 3-(2-chloropyridin-4-yl)-2-(4-fluorophenyl)-6,7-dihydropyrazolo[1,5-a]pyrazine-5(4H)-carboxylate

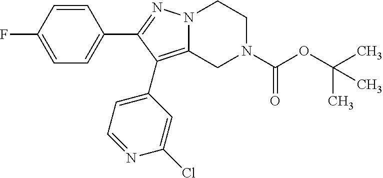 [SMILES: CC(C)(C)OC(=O)N1CCn2nc(-c3ccc(F)cc3)c(-c3ccnc(Cl)c3)c2C1]

To a mixture of THF (21 ml) and water (2.6 ml) was added tert-butyl 3-bromo-2-(4-fluorophenyl)-6,7-dihydropyrazolo[1,5-a]pyrazine-5(4H)-carboxylate (780 mg, 1.97 mmol, Intermediate 1) and then (2-chloropyridin-4-yl) boronic acid (341 mg, 2.17 mmol). After degassing of the solution with argon, potassium carbonate (816 mg, 5.91 mmol) and PdCl2(dppf)·CH₂Cl₂ (133 mg, 163 μmol; CAS-RN: [95464-05-4]) was added under a flow of argon and the mixture was heated under stirring at 110° C. for 1 hour under nitrogen atmosphere in the microwave. The reaction mixture was poured into water and extracted 3× with ethyl acetate. The combined organic layers were filtered with a hydrophobic filter and concentrated in vacuo to give a crude product. The crude product was purified by column chromatography on silica gel to yield 410 mg (97% purity, 47% yield) of the desired product.

LC-MS (Method 1): Rₜ=1.35 min; MS (ESIpos): m/z=429 [M+H]⁺.

¹H-NMR (400 MHz, DMSO-d6) δ [ppm]: 1.172 (0.58), 1.424 (16.00), 1.987 (1.07), 2.518 (1.91), 2.523 (1.28), 3.869 (1.10), 3.882 (1.93), 3.896 (1.25), 4.204 (1.45), 4.218 (2.23), 4.231 (1.23), 4.701 (4.06), 7.126 (1.53), 7.130 (1.56), 7.139 (1.54), 7.143 (1.60), 7.198 (2.12), 7.204 (0.76), 7.215 (0.97), 7.221 (4.53), 7.226 (0.93), 7.238 (0.87), 7.243 (2.78), 7.254 (3.32), 7.256 (3.03), 7.365 (2.04), 7.370 (0.96), 7.379 (2.28), 7.387 (1.87), 7.395 (0.75), 7.401 (1.56), 8.359 (2.31), 8.371 (2.23).

### Intermediate 94

### tert-butyl 2-bromo-3-(3-methyl-1H-pyrrolo[2,3-b]pyridin-4-yl)-6,7-dihydropyrazolo[1,5-a]pyrazine-5(4H)-carboxylate

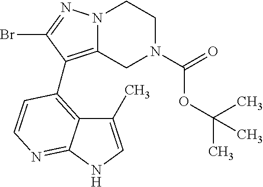 [SMILES: Cc1c[nH]c2nccc(-c3c(Br)nn4c3CN(C(=O)OC(C)(C)C)CC4)c12]

A solution of tert-butyl 2-bromo-3-iodo-6,7-dihydropyrazolo[1,5-a]pyrazine-5(4H)-carboxylate (1.66 g, 99% purity, 3.83 mmol, Intermediate 67) and (3-methyl-1H-pyrrolo[2,3-b]pyridin-4-yl) boronic acid (675 mg, 3.83 mmol, Intermediate 245) in 1,4-dioxane (17 ml) and water (1.7 ml) was degassed with argon for 15 min, sodium carbonate (1.02 g, 9.59 mmol) and PdCl2(dppf) (281 mg, 383 μmol; CAS-RN: [72287-26-4]) were added, degassed further for 5 min and stirred at 80° C. over night. The mixture was quenched with water and extracted twice with ethyl acetate. The organic phase was washed with brine, dried using a hydrophobic filter and concentrated in vacuo. The residue was purified by column chromatography to yield 686 mg (99% purity, 41% yield) of the desired product.

LC-MS (Method 1): Rₜ=1.01 min; MS (ESIpos): m/z=434 [M+H]⁺

¹H-NMR (400 MHz, DMSO-d6) δ [ppm]: 1.065 (0.66), 1.154 (4.08), 1.171 (8.64), 1.189 (4.30), 1.373 (7.07), 1.425 (1.28), 1.891 (15.56), 1.893 (16.00), 1.987 (15.06), 2.518 (1.72), 2.522 (1.08), 3.941 (0.52), 3.998 (1.15), 4.016 (3.16), 4.034 (3.01), 4.052 (1.02), 4.172 (1.87), 4.185 (3.42), 4.198 (1.74), 4.346 (2.11), 6.809 (5.49), 6.821 (5.54), 7.264 (2.78), 8.200 (6.27), 8.212 (5.82), 11.502 (2.17).

### Intermediate 95

### tert-butyl 2-bromo-3-(3-methylpyridin-4-yl)-6,7-dihydropyrazolo[1,5-a]pyrazine-5(4H)-carboxylate

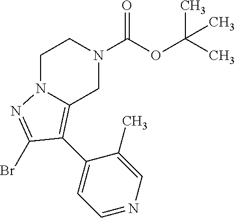 [SMILES: Cc1cnccc1-c1c(Br)nn2c1CN(C(=O)OC(C)(C)C)CC2]

A solution of 2-bromo-3-iodo-6,7-dihydropyrazolo[1,5-a]pyrazine-5(4H)-carboxylate (200 mg, 467 μmol, Intermediate 67) and (3-methylpyridin-4-yl) boronic acid (64.0 mg, 467 μmol) in 1,4-dioxane (2 ml) and water (0.5 ml) was degassed with argon for 15 min, sodium carbonate (99.0 mg, 934 μmol) and PdCl2(dppf) (34.2 mg, 46.7 μmol; CAS-RN: [72287-26-4]) were added, degassed further for 5 min and stirred at 80° C. for 16 h. The mixture was quenched with water and extracted twice with ethyl acetate. The organic phase was washed with brine, dried using a hydrophobic filter and concentrated in vacuo. The residue was purified by column chromatography to yield 64.3 mg (95% purity, 33% yield) of the desired product.

LC-MS (Method 1): Rₜ=0.78 min; MS (ESIpos): m/z=329.9 [M+H]⁺

¹H-NMR (400 MHz, DMSO-d6) δ [ppm]: 1.154 (1.43), 1.172 (2.90), 1.190 (1.45), 1.332 (1.16), 1.396 (14.65), 1.444 (0.84), 1.988 (5.33), 2.166 (16.00), 2.518 (4.91), 2.523 (2.99), 3.842 (1.31), 3.855 (1.75), 3.999 (0.41), 4.017 (1.23), 4.035 (1.18), 4.053 (0.40), 4.151 (2.08), 4.165 (3.74), 4.178 (1.72), 4.344 (0.71), 4.431 (2.52), 4.472 (1.07), 7.177 (2.90), 7.190 (2.98), 8.443 (2.92), 8.455 (2.88), 8.559 (4.62).

### Intermediate 96

### tert-butyl 2-bromo-3-(5-fluoro-1H-pyrrolo[2,3-b]pyridin-4-yl)-6,7-dihydropyrazolo[1,5-a]pyrazine-5(4H)-carboxylate

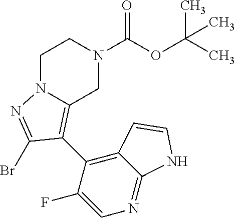 [SMILES: CC(C)(C)OC(=O)N1CCn2nc(Br)c(-c3c(F)cnc4[nH]ccc34)c2C1]

A solution of 2-bromo-3-iodo-6,7-dihydropyrazolo[1,5-a]pyrazine-5(4H)-carboxylate (200 mg, 467 μmol, Intermediate 67) and 5-fluoro-4-(4,4,5,5-tetramethyl-1,3,2-dioxaborolan-2-yl)-1H-pyrrolo[2,3-b]pyridine (122 mg, 467 μmol) in 1,4-dioxane (2 ml) and water (0.6 ml) was degassed with argon for 15 min, sodium carbonate (99.0 mg, 934 μmol) and PdCl2(dppf) (34.2 mg, 46.7 μmol; CAS-RN: [72287-26-4]) were added, degassed further for 5 min and stirred at 80° C. for 13 h. The mixture was quenched with water and extracted twice with ethyl acetate. The organic phase was washed with brine, dried using a hydrophobic filter and concentrated in vacuo. The residue was purified by column chromatography to yield 204 mg (70% purity, 70% yield) of the desired product.

LC-MS (Method 1): Rₜ=1.04 min; MS (ESIpos): m/z=437.9 [M+H]⁺

¹H-NMR (400 MHz, DMSO-d6) δ [ppm]: 1.065 (15.47), 1.155 (3.15), 1.392 (1.28), 1.425 (3.52), 1.434 (4.48), 2.518 (0.72), 2.523 (0.44), 3.565 (16.00), 3.939 (2.64), 4.404 (0.57), 7.632 (0.42).

### Intermediate 97

### tert-butyl 2-bromo-3-(3-fluoro-1H-pyrrolo[2,3-b]pyridin-4-yl)-6,7-dihydropyrazolo[1,5-a]pyrazine-5(4H)-carboxylate

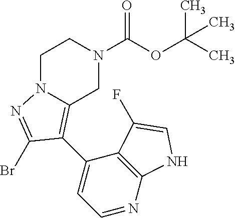 [SMILES: CC(C)(C)OC(=O)N1CCn2nc(Br)c(-c3ccnc4[nH]cc(F)c34)c2C1]

A solution of 2-bromo-3-iodo-6,7-dihydropyrazolo[1,5-a]pyrazine-5(4H)-carboxylate (200 mg, 467 μmol, Intermediate 67) and 3-fluoro-4-(4,4,5,5-tetramethyl-1,3,2-dioxaborolan-2-yl)-1H-pyrrolo[2,3-b]pyridine (122 mg, 467 μmol) in 1,4-dioxane (2 ml) and water (0.6 ml) was degassed with argon for 15 min, sodium carbonate (99.0 mg, 934 μmol) and PdCl2(dppf) (34.2 mg, 46.7 μmol; CAS-RN: [72287-26-4]) were added, degassed further for 5 min and stirred at 80° C. for 13 h. The mixture was quenched with water and extracted twice with ethyl acetate. The organic phase was washed with brine, dried using a hydrophobic filter and concentrated in vacuo. The residue was purified by column chromatography to yield 204 mg (75% purity, 75% yield) of the desired product.

LC-MS (Method 1): Rₜ=1.03 min; MS (ESIpos): m/z=435.9 [M+H]⁺

¹H-NMR (400 MHz, DMSO-d6) delta [ppm]: 1.064 (16.00), 1.155 (4.12), 1.332 (0.20), 1.391 (1.91), 1.425 (4.46), 1.434 (3.08), 2.522 (0.73), 3.564 (9.88), 3.772 (0.20), 3.786 (0.34), 3.799 (0.26), 3.941 (2.61), 4.043 (0.19), 4.057 (0.26), 4.070 (0.18), 4.091 (0.20), 4.190 (0.33), 4.203 (0.22), 4.404 (0.25), 4.469 (0.17), 4.491 (0.18), 4.559 (0.30), 6.279 (0.37), 6.986 (0.47), 6.998 (0.47), 7.529 (0.37), 7.943 (0.30), 8.312 (0.36), 8.324 (0.34), 11.693 (0.18).

### Intermediate 98

### tert-butyl 2-bromo-3-[2-(methylamino)pyridin-4-yl]-6,7-dihydropyrazolo[1,5-a]pyrazine-5(4H)-carboxylate

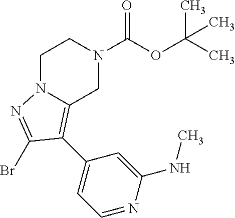 [SMILES: CNc1cc(-c2c(Br)nn3c2CN(C(=O)OC(C)(C)C)CC3)ccn1]

A solution of 2-bromo-3-iodo-6,7-dihydropyrazolo[1,5-a]pyrazine-5(4H)-carboxylate (200 mg, 467 μmol, Intermediate 67) and N-methyl-4-(4,4,5,5-tetramethyl-1,3,2-dioxaborolan-2-yl)pyridin-2-amine (109 mg, 467 μmol; CAS-RN: [1350913-084]) in 1,4-dioxane (2 ml) and water (0.6 ml) was degassed with argon for 15 min, sodium carbonate (99.0 mg, 934 μmol) and PdCl2(dppf) (34.2 mg, 46.7 μmol; CAS-RN: [72287-26-4]) were added, degassed further for 5 min and stirred at 80° C. for 13 h. The mixture was quenched with water and extracted twice with ethyl acetate. The organic phase was washed with brine, dried using a hydrophobic filter and concentrated in vacuo. The residue was purified by column chromatography to yield 284 mg (67% purity, 100% yield) of the desired product.

LC-MS (Method 1): Rₜ=0.72 min; MS (ESIpos): m/z=409.9 [M+H]⁺.

¹H-NMR (400 MHz, DMSO-d6) δ [ppm]: 1.064 (16.00), 1.154 (2.64), 1.408 (4.61), 1.424 (1.24), 1.434 (2.35), 2.770 (1.35), 2.782 (1.35), 3.564 (6.32), 3.843 (0.49), 3.939 (2.74), 4.129 (0.59), 4.638 (0.84), 6.438 (0.63), 8.014 (0.45), 8.028 (0.44).

### Intermediate 99

### tert-butyl 3-(2-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate

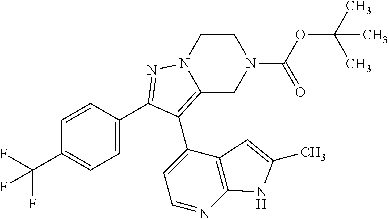 [SMILES: Cc1cc2c(-c3c(-c4ccc(C(F)(F)F)cc4)nn4c3CN(C(=O)OC(C)(C)C)CC4)ccnc2[nH]1]

A solution of tert-butyl 3-iodo-2-[4-(trifluoromethyl)phenyl]-6,7-dihydropyrazolo[1,5-a]pyrazine-5(4H)-carboxylate (150 mg, 304 μmol, Intermediate 88) and (2-methyl-1H-pyrrolo[2,3-b]pyridin-4-yl) boronic acid (53.5 mg, 304 μmol; CAS-RN: [1014614-07-3]) in 1,4-dioxane (2 ml) and water (0.6 ml) was degassed with argon for 15 min, sodium carbonate (64.5 mg, 608 μmol) and PdCl2(dppf) (22.3 mg, 30.4 μmol; CAS-RN: [72287-26-4]) were added, degassed further for 5 min and stirred at 80° C. for 16 h. The mixture was quenched with water and extracted twice with ethyl acetate. The organic phase was washed with brine, dried using a hydrophobic filter and concentrated in vacuo. The residue was purified by column chromatography to yield 90.5 mg (98% purity, 59% yield) of the desired product.

LC-MS (Method 1): Rₜ=1.25 min; MS (ESIpos): m/z=498 [M+H]⁺. 1H-NMR (400 MHz, DMSO-d6) δ[ppm]: 1.154 (4.41), 1.172 (9.50), 1.190 (4.74), 1.396 (5.13), 1.988 (16.00), 2.313 (5.23), 2.327 (0.58), 2.518 (1.12), 2.523 (0.75), 2.669 (0.41), 3.920 (0.69), 3.999 (1.18), 4.017 (3.49), 4.034 (3.35), 4.053 (1.07), 4.263 (0.84), 4.277 (1.40), 4.290 (0.71), 4.466 (0.43), 6.767 (0.74), 6.779 (0.74), 7.505 (0.87), 7.525 (1.24), 7.596 (2.36), 7.617 (1.48), 8.077 (1.59), 8.089 (1.60), 11.609 (0.77).

### Intermediate 100

### tert-butyl 3-(3-cyclopropylpyridin-4-yl)-2-[4-(trifluoromethyl)phenyl]-6,7-dihydropyrazolo[1,5-a]pyrazine-5(4H)-carboxylate

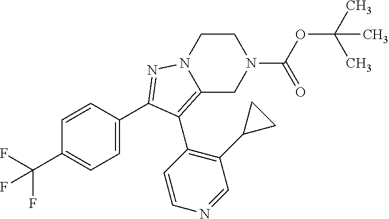 [SMILES: CC(C)(C)OC(=O)N1CCn2nc(-c3ccc(C(F)(F)F)cc3)c(-c3ccncc3C3CC3)c2C1]

A solution of 3-iodo-2-[4-(trifluoromethyl)phenyl]-6,7-dihydropyrazolo[1,5-a]pyrazine-5(4H)-carboxylate (150 mg, 304 μmol, Intermediate 88) and 3-cyclopropyl-4-(4,4,5,5-tetramethyl-1,3,2-dioxaborolan-2-yl)pyridine (74.5 mg, 304 μmol; CAS-RN: [2096333-32-1]) in 1,4-dioxane (2 ml) and water (0.6 ml) was degassed with argon for 15 min, sodium carbonate (64.5 mg, 608 μmol) and PdCl2(dppf) (22.3 mg, 30.4 μmol; CAS-RN: [72287-26-4]) were added, degassed further for 5 min and stirred at 80° C. for 16 h. The mixture was quenched with water and extracted twice with ethyl acetate. The organic phase was washed with brine, dried using a hydrophobic filter and concentrated in vacuo. The residue was purified by column chromatography to yield 127 mg (98% purity, 84% yield).

LC-MS (Method 1): Rₜ=1.19 min; MS (ESIpos): m/z=485 [M+H]⁺. 1H-NMR (400 MHz, DMSO-d6) δ [ppm]: 0.744 (0.47), 0.756 (0.41), 0.836 (0.44), 1.065 (15.68), 1.154 (4.04), 1.172 (8.08), 1.190 (4.15), 1.411 (7.45), 1.987 (16.00), 2.523 (0.76), 3.939 (1.34), 3.999 (1.35), 4.017 (3.76), 4.035 (3.70), 4.053 (1.21), 4.258 (0.86), 4.272 (1.57), 4.284 (0.87), 4.458 (0.74), 7.169 (1.12), 7.181 (1.12), 7.499 (1.57), 7.520 (1.92), 7.650 (2.14), 7.671 (1.62), 8.209 (1.81), 8.396 (1.58), 8.408 (1.51).

### Intermediate 101

### tert-butyl 2-(4-chloro-2-fluoroanilino)-6,7-dihydropyrazolo[1,5-a]pyrazine-5(4H)-carboxylate

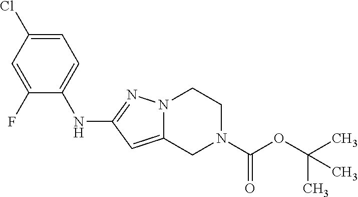 [SMILES: CC(C)(C)OC(=O)N1CCn2nc(Nc3ccc(Cl)cc3F)cc2C1]

Under argon tert-butyl 2-amino-6,7-dihydropyrazolo[1,5-a]pyrazine-5(4H)-carboxylate (500 mg, 2.10 mmol; CAS-RN: [1209487-56-8]) was dissolved in 1,4-dioxane (5 ml), 1-bromo-4-chloro-2-fluorobenzene (879 mg, 4.20 mmol; CAS-RN: [1996-29-8]) was added and the mixture was degassed with argon for 5 min. Potassiumcarbonate (580 mg, 4.20 mmol), XPhos (40.0 mg, 83.9 μmol; CAS-RN: [564483-18-7]) and Tris-(dibenzylidenacetone) dipalladium (76.9 mg, 83.9 μmol; CAS-RN: [51364-51-3]) were added and heated to 100° C. over night. Water was added to the reaction mixture, extracted twice into ethyl acetate, dried using a hydrophobic filter and evaporated in vacuo. The crude product was purified on 25 g silica (eluent: hexane/ethyl acetate 0-40%) to yield 649 mg (97% purity, 82% yield) of the desired product.

LC-MS (Method 1): Rₜ=1.36 min; MS (ESIpos): m/z=367 [M+H]⁺.

¹H-NMR (400 MHz, DMSO-d6) δ [ppm]: 1.433 (16.00), 3.798 (0.69), 3.811 (0.46), 3.963 (0.57), 3.977 (0.83), 3.989 (0.41), 4.537 (0.79), 5.810 (1.66), 7.286 (0.60), 7.292 (0.56), 7.315 (0.57), 7.321 (0.56), 8.124 (0.49), 8.147 (0.69), 8.170 (0.46), 8.455 (0.61), 8.461 (0.59).

### Intermediate 102

### tert-butyl 3-bromo-2-(4-chloro-2-fluoroanilino)-6,7-dihydropyrazolo[1,5-a]pyrazine-5(4H)-carboxylate

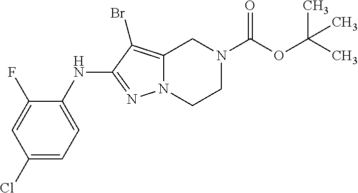 [SMILES: CC(C)(C)OC(=O)N1CCn2nc(Nc3ccc(Cl)cc3F)c(Br)c2C1]

Under argon tert-butyl 2-(4-chloro-2-fluoroanilino)-6,7-dihydropyrazolo[1,5-a]pyrazine-5(4H)-carboxylate (645 mg, 1.76 mmol, Intermediate 101) was dissolved in DCM (8.7 ml), 1-bromopyrrolidine-2,5-dione (313 mg, 1.76 mmol) was added and the mixture was allowed to stir at RT for 2 h. Water and sodiumhydrogencarbonate solution were added and the mixture was extracted twice into DCM, dried using a hydrophobic filter and then the solvents were removed in vacuo. The crude material was purified on 10 g silica (eluent: hexane/ethyl acetate 0-40%) to yield 636 mg (98% purity, 80% yield).

LC-MS (Method 1): Rₜ=1.56 min; MS (ESIpos): m/z=447 [M+H]⁺.

¹H-NMR (400 MHz, DMSO-d6) δ [ppm]: 1.445 (16.00), 3.828 (0.67), 3.841 (0.45), 4.003 (0.59), 4.017 (0.89), 4.031 (0.42), 4.462 (1.37), 7.113 (0.43), 7.285 (0.51), 7.307 (0.73), 7.324 (0.70), 7.331 (0.94), 7.353 (0.64), 7.359 (0.61), 7.658 (0.67), 7.662 (0.65).

### Intermediate 103

### tert-butyl 2-(4-chloro-2-fluoroanilino)-3-(3-methyl-1H-pyrrolo[2,3-b]pyridin-4-yl)-6,7-dihydropyrazolo[1,5-a]pyrazine-5(4H)-carboxylate

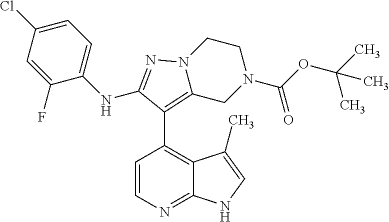 [SMILES: Cc1c[nH]c2nccc(-c3c(Nc4ccc(Cl)cc4F)nn4c3CN(C(=O)OC(C)(C)C)CC4)c12]

tert-butyl 3-bromo-2-(4-chloro-2-fluoroanilino)-6,7-dihydropyrazolo[1,5-a]pyrazine-5(4H)-carboxylate (120 mg, 269 μmol, Intermediate 102) and 3-methyl-4-(4,4,5,5-tetramethyl-1,3,2-dioxaborolan-2-yl)-1H-pyrrolo[2,3-b]pyridine (76.4 mg, 296 μmol; CAS-RN: [2304634-64-6]) were dissolved in 1,4-dioxane (2.6) and degassed with argon for 5 min. sodiumcarbonate (650 μl, 2.0 M, 1.3 mmol) and PdCl2(PCy3)2 (19.9 mg, 26.9 μmol; CAS-RN: [29934-17-6]) as catalyst were added, degassed again with argon for 5 min and heated in the microwave oven to 150° C. for 20 min. The reaction was quenched with water, extracted twice into ethyl acetate, dried using a hydrophobic filter and purified via reversed phase-HPLC (acidic conditions) to yield 56.0 mg (92% purity, 39% yield) of the desired product.

LC-MS (Method 1): Rₜ=1.29 min; MS (ESIpos): m/z=497 [M+H]⁺.

¹H-NMR (400 MHz, DMSO-d6) δ [ppm]: 1.212 (0.45), 1.235 (0.53), 1.385 (6.85), 1.433 (16.00), 1.784 (0.44), 1.948 (10.86), 2.518 (4.13), 2.523 (2.76), 2.678 (0.46), 3.784 (0.53), 3.797 (0.83), 3.811 (0.60), 3.962 (0.79), 3.976 (1.05), 3.990 (0.67), 4.092 (0.90), 4.108 (1.77), 4.120 (2.20), 4.132 (0.93), 4.346 (1.43), 4.536 (0.75), 5.809 (1.59), 6.839 (4.78), 6.851 (4.72), 7.048 (1.39), 7.053 (1.51), 7.069 (1.48), 7.075 (1.65), 7.187 (2.19), 7.193 (2.18), 7.212 (2.96), 7.218 (2.91), 7.231 (2.44), 7.240 (3.08), 7.247 (2.72), 7.286 (0.59), 7.293 (0.55), 7.315 (0.55), 7.322 (0.53), 7.666 (0.74), 8.122 (0.50), 8.146 (0.77), 8.155 (5.42), 8.167 (5.32), 8.453 (0.58), 8.459 (0.56), 11.424 (2.23), 11.430 (2.17).

### Intermediate 104

### tert-butyl 2-(3-fluoro-4-methoxyanilino)-6,7-dihydropyrazolo[1,5-a]pyrazine-5(4H)-carboxylate

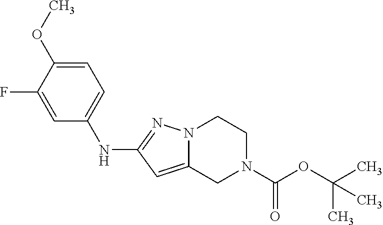 [SMILES: COc1ccc(Nc2cc3n(n2)CCN(C(=O)OC(C)(C)C)C3)cc1F]

Under argon tert-butyl 2-amino-6,7-dihydropyrazolo[1,5-a]pyrazine-5(4H)-carboxylate (500 mg. 2.10 mmol; CAS-RN: [1209487-56-8]) was dissolved in 1,4-dioxane (5 ml), 4-bromo-2-fluoro-1-methoxybenzene (860 mg, 4.20 mmol; CAS-RN: [2357-52-0]) was added and the mixture was degassed with argon for 5 min. Potassiumcarbonate (580 mg, 4.20 mmol; CAS-RN: [584-08-7]), XPhos (40.0 mg, 83.9 μmol; CAS-RN: [564483-18-7]) and Pd2(dba)3 (76.9 mg, 83.9 μmol; CAS-RN: [51364-51-3]) were added and heated to 100° C. for 16 h. Water was added to the reaction mixture, extracted twice into ethyl acetate, dried using a hydrophobic filter and evaporated in vacuo. The crude product was purified on 10 g silica (eluent: hexane/EtoAc 0-45%) to yield 670 mg (90% purity, 79% yield) of the desired product.

LC-MS (Method 1): Rₜ=1.17 min; MS (ESIpos): m/z=363 [M+H]⁺.

¹H-NMR (400 MHz, DMSO-d6) δ [ppm]: 1.154 (1.53), 1.172 (3.14), 1.190 (1.49), 1.418 (0.74), 1.432 (16.00), 1.447 (0.56), 1.988 (5.26), 3.736 (7.28), 3.789 (0.65), 3.803 (0.46), 3.938 (0.56), 3.951 (0.79), 4.017 (1.09), 4.035 (1.04), 4.523 (0.79), 5.628 (1.58), 6.980 (1.07), 6.983 (1.40), 6.989 (0.53), 6.999 (0.63), 7.362 (0.47), 8.456 (0.90).

### Intermediate 105

### tert-butyl 3-bromo-2-(3-fluoro-4-methoxyanilino)-6,7-dihydropyrazolo[1,5-a]pyrazine-5(4H)-carboxylate

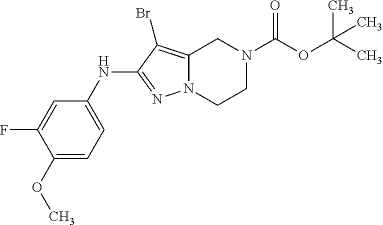 [SMILES: COc1ccc(Nc2nn3c(c2Br)CN(C(=O)OC(C)(C)C)CC3)cc1F]

Under argon tert-butyl 2-(3-fluoro-4-methoxyanilino)-6,7-dihydropyrazolo[1,5-a]pyrazine-5(4H)-carboxylate (670 mg, 1.85 mmol, Intermediate 104) was dissolved in DCM (9 ml), 1-bromopyrrolidine-2,5-dione (329 mg, 1.85 mmol) was added and the mixture was allowed to stir at RT for 2 h. Water and sodiumhydrogencarbonate solution were added and the mixture was extracted twice into DCM, dried using a hydrophobic filter and then the solvents were removed in vacuo. The crude material was purified on 10 g silica (eluent: hexane/ethyl acetate 0-40%) to yield 325 mg (98% purity, 39% yield).

LC-MS (Method 1): Rₜ=1.34 min; MS (ESIpos): m/z=441 [M+H]⁺.

¹H-NMR (400 MHz, DMSO-d6) δ [ppm]: 1.443 (16.00), 2.518 (0.44), 3.747 (7.72), 3.815 (0.69), 3.828 (0.47), 3.979 (0.59), 3.993 (0.86), 4.007 (0.43), 4.438 (1.32), 6.986 (0.43), 7.009 (0.67), 7.034 (0.53), 7.167 (0.41), 7.961 (1.11).

### Intermediate 106

### tert-butyl 2-(3-fluoro-4-methoxyanilino)-3-(3-methyl-1H-pyrrolo[2,3-b]pyridin-4-yl)-6,7-dihydropyrazolo[1,5-a]pyrazine-5(4H)-carboxylate

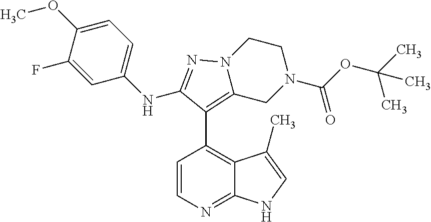 [SMILES: COc1ccc(Nc2nn3c(c2-c2ccnc4[nH]cc(C)c24)CN(C(=O)OC(C)(C)C)CC3)cc1F]

3-bromo-2-(3-fluoro-4-methoxyanilino)-6,7-dihydropyrazolo[1,5-a]pyrazine-5(4H)-carboxylate (120 mg, 272 μmol, Intermediate 105) and 3-methyl-4-(4,4,5,5-tetramethyl-1,3,2-dioxaborolan-2-yl)-1H-pyrrolo[2,3-b]pyridine (77.2 mg, 299 μmol; CAS-RN: [2304634-64-6]) were dissolved in 1,4-dioxane (2.6 ml) and degassed with argon for 5 min. sodiumcarbonate (650 μl, 2.0 M, 1.3 mmol) and PdCl2(PCy3)2 (20.1 mg, 27.2 μmol; CAS-RN: [29934-17-6]) as catalyst were added, degassed again with argon for 5 min and heated in the microwave oven to 150° C. for 20 min. The reaction was quenched with water, extracted twice into ethyl acetate, dried using a hydrophobic filter and purified via reversed phase-HPLC (acidic conditions) to yield 50.0 mg (99% purity, 37% yield) of the desired product.

LC-MS (Method 1): Rₜ=1.1 min; MS (ESIpos): m/z=493 [M+H]⁺. 1H-NMR (400 MHz, DMSO-d6) δ [ppm]: 1.377 (3.38), 1.916 (5.94), 2.518 (2.09), 2.523 (1.38), 3.716 (16.00), 4.070 (1.02), 4.082 (1.30), 4.304 (0.87), 6.812 (2.89), 6.824 (2.80), 6.904 (0.95), 6.927 (1.57), 6.951 (1.18), 7.089 (0.93), 7.093 (0.94), 7.096 (0.83), 7.112 (0.70), 7.115 (0.71), 7.118 (0.62), 7.194 (1.25), 7.389 (0.86), 7.395 (0.80), 7.426 (0.83), 7.432 (0.80), 7.640 (2.31), 8.169 (2.88), 8.181 (2.65), 11.367 (1.28), 11.372 (1.24).

### Intermediate 107

### tert-butyl 2-[4-(trifluoromethyl) anilino]-6,7-dihydropyrazolo[1,5-a]pyrazine-5(4H)-carboxylate

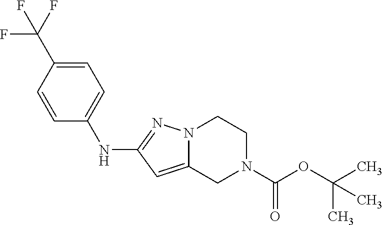 [SMILES: CC(C)(C)OC(=O)N1CCn2nc(Nc3ccc(C(F)(F)F)cc3)cc2C1]

Under argon tert-butyl 2-amino-6,7-dihydropyrazolo[1,5-a]pyrazine-5(4H)-carboxylate (500 mg, 2.10 mmol; CAS-RN: [1209487-56-8]) was dissolved in 1,4-dioxane (5 ml), 1-bromo-4-(trifluoromethyl)benzene (944 mg, 4.20 mmol; CAS-RN: [402-43-7]) was added and the mixture was degassed with argon for 5 min. Potassium carbonate (580 mg, 4.20 mmol), XPhos (40.0 mg, 83.9 μmol; CAS-RN: [564483-18-7]) and Tris-(dibenzylidenacetone) dipalladium (76.9 mg, 83.9 μmol; CAS-RN: [51364-51-3]) were added and heated to 100° C. for 16 h. Water was added to the reaction mixture, extracted twice into ethyl acetate, dried using a hydrophobic filter and evaporated in vacuo. The crude product was purified on 10 g silica (eluent: hexane/ethyl acetate 0-40%) to yield 410 mg (98% purity, 50% yield) of the desired product

LC-MS (Method 1): Rₜ=1.35 min; MS (ESIpos): m/z=383 [M+H]⁺.

¹H-NMR (400 MHz, DMSO-d6) δ [ppm]: 1.154 (0.80), 1.172 (1.75), 1.190 (0.85), 1.437 (16.00), 1.988 (2.57), 3.808 (0.64), 3.822 (0.43), 3.978 (0.54), 3.992 (0.78), 4.017 (0.61), 4.034 (0.57), 4.554 (0.79), 5.760 (1.00), 5.763 (1.61), 7.485 (2.19), 7.490 (2.33), 9.049 (1.07).

### Intermediate 108

### tert-butyl 3-bromo-2-[4-(trifluoromethyl) anilino]-6,7-dihydropyrazolo[1,5-a]pyrazine-5(4H)-carboxylate

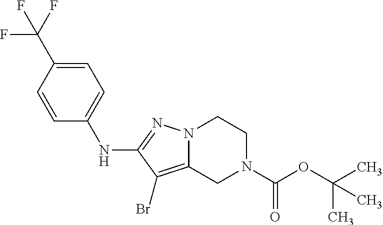 [SMILES: CC(C)(C)OC(=O)N1CCn2nc(Nc3ccc(C(F)(F)F)cc3)c(Br)c2C1]

Under argon tert-butyl 2-[4-(trifluoromethyl) anilino]-6,7-dihydropyrazolo[1,5-a]pyrazine-5(4H)-carboxylate (450 mg, 1.18 mmol, Intermediate 107) was dissolved in DCM (5.7 ml), 1-bromopyrrolidine-2,5-dione (209 mg, 1.18 mmol) was added and the mixture was allowed to stir at RT for 90 min. Water and aqueous sodiumhydrogencarbonate solution were added, extracted twice into DCM, dried using a hydrophobic filter and the solvents were removed in vacuo. The crude material was purified on 10 g silica (eluent: hexane/ethyl acetate 0-20%) to yield 424 mg (98% purity, 77% yield) of the desired product.

LC-MS (Method 1): Rₜ=1.48 min; MS (ESIpos): m/z=461 [M+H]⁺

¹H-NMR (400 MHz, DMSO-d6) δ [ppm]: 1.035 (1.32), 1.052 (2.42), 1.070 (1.40), 1.448 (16.00), 2.518 (0.42), 3.836 (0.69), 3.850 (0.47), 4.025 (0.60), 4.039 (0.87), 4.051 (0.45), 4.469 (1.40), 7.436 (0.70), 7.459 (1.32), 7.498 (1.44), 7.521 (0.71), 8.551 (1.28).

### Intermediate 109

### tert-butyl 3-(3-methyl-1H-pyrrolo[2,3-b]pyridin-4-yl)-2-[4-(trifluoromethyl) anilino]-6,7-dihydropyrazolo[1,5-a]pyrazine-5(4H)-carboxylate

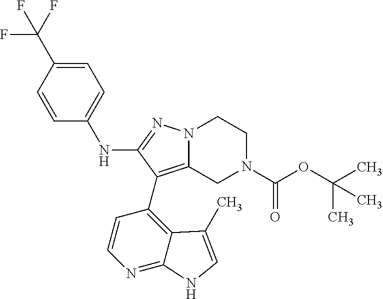 [SMILES: Cc1c[nH]c2nccc(-c3c(Nc4ccc(C(F)(F)F)cc4)nn4c3CN(C(=O)OC(C)(C)C)CC4)c12]

Under argon tert-butyl 3-bromo-2-[4-(trifluoromethyl) anilino]-6,7-dihydropyrazolo[1,5-a]pyrazine-5(4H)-carboxylate (134 mg, 290 μmol, Intermediate 108) was dissolved in 1,4-dioxane (5 ml), 3-methyl-4-(4,4,5,5-tetramethyl-1,3,2-dioxaborolan-2-yl)-1H-pyrrolo[2,3-b]pyridine (150 mg, 581 μmol; CAS-RN: [2304634-64-6]) was added and the mixture was degassed with argon for 5 min. Potassium carbonate (80.3 mg, 581 μmol), XPhos (5.53 mg, 11.6 μmol; CAS-RN: [564483-18-7]) and Tris-(dibenzylidenacetone) dipalladium (10.6 mg, 11.6 μmol; CAS-RN: [51364-51-3]) were added and heated to 150° C. for 20 min. Water was added to the reaction mixture, extracted twice into ethyl acetate, dried using a hydrophobic filter and evaporated in vacuo. The crude product was purified on reversed phase HPLC (acidic conditions) to yield 42.0 mg (82% purity, 23% yield) of the desired product.

LC-MS (Method 1): Rₜ=1.29 min; MS (ESIpos): m/z=513 [M+H]⁺.

¹H-NMR (400 MHz, DMSO-d6) δ [ppm]: 1.381 (2.27), 1.437 (16.00), 1.906 (3.45), 2.518 (1.82), 2.523 (1.12), 3.795 (0.47), 3.808 (0.82), 3.822 (0.58), 3.977 (0.73), 3.992 (1.01), 4.005 (0.54), 4.124 (0.88), 4.328 (0.56), 4.553 (0.92), 5.762 (1.61), 6.826 (1.97), 6.838 (1.97), 7.199 (0.81), 7.412 (1.03), 7.435 (2.39), 7.466 (1.99), 7.484 (2.92), 7.490 (3.12), 8.171 (1.83), 8.183 (1.72), 8.281 (1.57), 9.049 (1.14), 11.387 (0.84).

### Intermediate 110

### tert-butyl 2-(4-chloro-2-fluoroanilino)-3-(pyridin-4-yl)-6,7-dihydropyrazolo[1,5-a]pyrazine-5(4H)-carboxylate

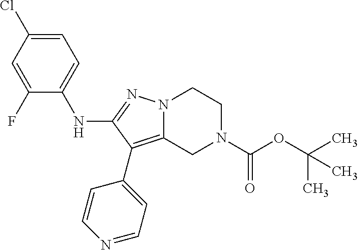 [SMILES: CC(C)(C)OC(=O)N1CCn2nc(Nc3ccc(Cl)cc3F)c(-c3ccncc3)c2C1]

tert-butyl 3-bromo-2-(4-chloro-2-fluoroanilino)-6,7-dihydropyrazolo[1,5-a]pyrazine-5(4H)-carboxylate (132 mg, 296 μmol, Intermediate 102) and pyridin-4-ylboronic acid (40.0 mg, 326 μmol; CAS-RN: [1692-15-5]) were dissolved in 1,4-dioxane (2.8 ml), 2 mol/l aqueous sodiumcarbonate solution was added and degassed with argon for 5 min. PdCl2(PCy3)2 (21.9 mg, 29.6 μmol; CAS-RN: [29934-17-6]) was added, degassed again with argon for 5 min and heated in the microwave oven to 150° C. for 20 min. The reaction was quenched with water, extracted twice into ethyl acetate, dried using a hydrophobic filter and evaporated in vacuo. The residue was dissolved in DMSO and purified via reversed phase HPLC (basic conditions) to yield 70.0 mg (98% purity, 52% yield) of the desired product.

LC-MS (Method 1): Rₜ=0.97 min; MS (ESIpos): m/z=444 [M+H]⁺.

¹H-NMR (400 MHz, DMSO-d6) δ [ppm]: 1.418 (16.00), 2.518 (1.31), 2.523 (0.88), 3.311 (0.41), 3.863 (1.12), 3.877 (2.14), 3.890 (1.44), 4.068 (1.68), 4.082 (2.49), 4.095 (1.27), 4.733 (3.59), 6.993 (1.00), 6.995 (0.96), 6.998 (1.05), 7.001 (0.91), 7.015 (1.30), 7.017 (1.25), 7.020 (1.41), 7.023 (1.23), 7.133 (1.22), 7.155 (1.87), 7.178 (0.91), 7.286 (1.97), 7.292 (1.86), 7.314 (1.94), 7.321 (1.82), 7.348 (4.76), 7.352 (2.74), 7.359 (2.87), 7.363 (4.59), 7.913 (1.89), 7.917 (1.86), 8.525 (5.34), 8.528 (2.84), 8.536 (2.93), 8.540 (4.86).

### Intermediate 111

### tert-butyl 2-(3-fluoro-4-methoxyanilino)-3-(pyridin-4-yl)-6,7-dihydropyrazolo[1,5-a]pyrazine-5(4H)-carboxylate

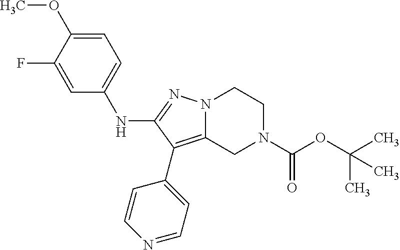 [SMILES: COc1ccc(Nc2nn3c(c2-c2ccncc2)CN(C(=O)OC(C)(C)C)CC3)cc1F]

Tert-butyl 3-bromo-2-(3-fluoro-4-methoxyanilino)-6,7-dihydropyrazolo[1,5-a]pyrazine-5(4H)-carboxylate (192 mg, 435 μmol, Intermediate 105) and pyridin-4-ylboronic acid (58.8 mg, 479 μmol; CAS-RN: [1692-15-5]) were dissolved in 1,4-dioxane (4.1 ml), 2 mol/l aqueous sodiumcarbonate solution was added and degassed with argon for 5 min. PdCl2(PCy3)2 (32.1 mg, 43.5 μmol; CAS-RN: [29934-17-6]) was added, degassed again with argon for 5 min and heated in the microwave oven to 150° C. for 20 min. The reaction was quenched with water, extracted twice into ethyl acetate, dried using a hydrophobic filter and evaporated in vacuo. The residue was dissolved in DMSO and purified via reversed phase HPLC (basic conditions) to yield 110 mg (94% purity, 54% yield) of the desired product.

LC-MS (Method 1): Rₜ=0.84 min; MS (ESIpos): m/z=440 [M+H]⁺.

¹H-NMR (400 MHz, DMSO-d6) δ [ppm]: 1.413 (11.71), 1.432 (7.27), 2.523 (0.49), 3.730 (16.00), 3.736 (3.23), 3.849 (0.92), 3.863 (1.76), 3.876 (1.18), 4.047 (1.36), 4.061 (2.03), 4.073 (1.01), 4.681 (2.88), 5.628 (0.67), 6.961 (1.83), 6.966 (1.38), 6.972 (2.55), 6.976 (2.88), 7.169 (0.79), 7.207 (0.84), 7.332 (3.39), 7.336 (2.15), 7.343 (2.14), 7.347 (3.45), 8.012 (1.66), 8.455 (0.43), 8.544 (3.74), 8.548 (2.22), 8.555 (2.16), 8.559 (3.55).

### Intermediate 112

### tert-butyl 3-(pyridin-4-yl)-2-[4-(trifluoromethyl) anilino]-6,7-dihydropyrazolo[1,5-a]pyrazine-5(4H)-carboxylate

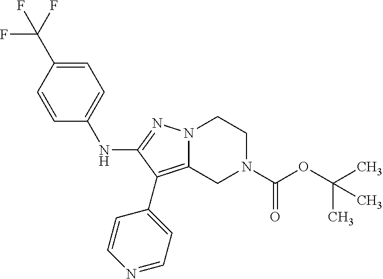 [SMILES: CC(C)(C)OC(=O)N1CCn2nc(Nc3ccc(C(F)(F)F)cc3)c(-c3ccncc3)c2C1]

tert-butyl 3-bromo-2-[4-(trifluoromethyl) anilino]-6,7-dihydropyrazolo[1,5-a]pyrazine-5(4H)-carboxylate (134 mg, 290 μmol, Intermediate 108) and pyridin-4-ylboronic acid (39.3 mg, 320 μmol; CAS-RN: [1692-15-5]) were dissolved in 1,4-dioxane (2.8 ml), 2 mol/l aqueous sodiumcarbonate solution was added and degassed with argon for 5 min. PdCl2(PCy3)2 (21.4 mg, 29.0 μmol; CAS-RN: [29934-17-6]) as catalyst was added, degassed again with argon for 5 min and heated in the microwave oven to 150° C. for 20 min. The reaction was quenched with water, extracted twice into ethyl acetate, dried using a hydrophobic filter and evaporated in vacuo. The residue was dissolved in DMSO and purified via reversed phase

HPLC (basic conditions) to yield 72.0 mg (98% purity, 53% yield) of the desired product.

LC-MS (Method 1): Rₜ=1.0 min; MS (ESIpos): m/z=460 [M+H]⁺.

¹H-NMR (400 MHz, DMSO-d6) δ [ppm]: 1.420 (16.00), 2.472 (0.43), 2.477 (0.54), 2.518 (1.05), 2.522 (0.53), 3.873 (1.26), 3.886 (2.36), 3.900 (1.56), 4.093 (1.83), 4.107 (2.76), 4.120 (1.42), 4.724 (4.10), 7.237 (2.96), 7.259 (3.47), 7.337 (4.87), 7.341 (2.91), 7.348 (2.97), 7.352 (4.82), 7.444 (4.01), 7.465 (3.25), 8.547 (4.93), 8.551 (2.92), 8.558 (2.87), 8.562 (4.66), 8.618 (2.76).

The following intermediates were prepared analogous to the preparation of Intermediate 69 starting from the corresponding intermediates described in the table.

| | Structure |
|---|---|
| | IUPAC-Name |
| | LC-MS (method): Retention time; Mass found |
| Intermediate | ¹H-NMR |
| 113 | 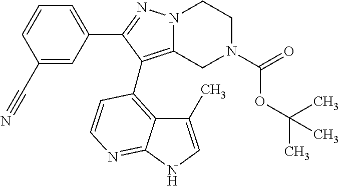 [SMILES: Cc1c[nH]c2nccc(-c3c(-c4cccc(C#N)c4)nn4c3CN(C(=O)OC(C)(C)C)CC4)c12] |
| | tert-butyl 2-(3-cyanophenyl)-3-(3-methyl-1H-pyrrolo[2,3-b]pyridin-4-yl)-6,7- |
| | dihydropyrazolo[1,5-a]pyrazine-5(4H)-carboxylate |
| | Produced from tert-butyl 2-bromo-3-(3-methyl-1H-pyrrolo[2,3-b]pyridin-4-yl)- |
| | 6,7-dihydropyrazolo[1,5-a]pyrazine-5(4H)-carboxylate (Intermediate 94) and (3- |
| | cyanophenyl)boronic acid. |
| | LC-MS (Method 1): Rₜ = 1.1 min; m/z = 455 [M + H]⁺ |
| | ¹H-NMR (400 MHz, DMSO-d6) δ [ppm]: 1.232 (0.57), 1.380 (16.00), 1.425 |
| | (4.97), 1.429 (4.58), 1.457 (1.50), 1.718 (13.40), 1.891 (0.78), 2.003 (0.56), |
| | 2.318 (1.26), 2.337 (1.23), 2.518 (12.19), 2.523 (8.11), 2.678 (1.31), 3.849 |
| | (1.17), 3.951 (1.45), 4.209 (1.80), 4.213 (1.80), 4.267 (5.61), 4.279 (9.69), |
| | 4.292 (5.69), 4.350 (6.91), 4.391 (1.61), 4.491 (1.57), 4.496 (1.54), 4.541 |
| | (0.41), 6.823 (0.43), 6.889 (10.13), 6.900 (9.98), 7.220 (6.29), 7.384 (5.69), |
| | 7.404 (12.58), 7.424 (7.94), 7.469 (0.78), 7.480 (1.52), 7.490 (1.35), 7.498 |
| | (1.51), 7.506 (1.45), 7.524 (1.77), 7.550 (8.77), 7.554 (10.35), 7.557 (7.62), |
| | 7.571 (6.43), 7.575 (7.62), 7.578 (5.75), 7.627 (0.69), 7.640 (6.03), 7.644 |
| | (8.90), 7.647 (6.86), 7.659 (5.07), 7.663 (8.20), 7.666 (6.20), 7.685 (9.46), |
| | 7.688 (13.68), 7.692 (7.00), 8.232 (6.64), 8.243 (6.32), 11.509 (6.67), 11.514 |
| | (6.61). |
| 114 | 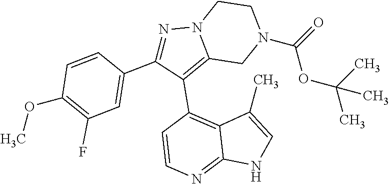 [SMILES: COc1ccc(-c2nn3c(c2-c2ccnc4[nH]cc(C)c24)CN(C(=O)OC(C)(C)C)CC3)cc1F] |
| | tert-butyl 2-(3-fluoro-4-methoxyphenyl)-3-(3-methyl-1H-pyrrolo[2,3- |
| | b]pyridin-4-yl)-6,7-dihydropyrazolo[1,5-a]pyrazine-5(4H)-carboxylate |
| | Produced from tert-butyl 2-bromo-3-(3-methyl-1H-pyrrolo[2,3-b]pyridin-4-yl)- |
| | 6,7-dihydropyrazolo[1,5-a]pyrazine-5(4H)-carboxylate (Intermediate 94) and |
| | (3-fluoro-4-methoxyphenyl)boronic acid. |
| | LC-MS (Method 1): Rₜ = 1.12 min; m/z = 478 [M + H]⁺ |
| | ¹H-NMR (400 MHz, DMSO-d6) δ [ppm]: 1.154 (0.51), 1.172 (1.01), 1.190 |
| | (0.50), 1.376 (1.20), 1.424 (0.47), 1.729 (1.27), 1.988 (1.82), 2.518 (1.31), |
| | 2.523 (0.84), 3.735 (6.76), 3.865 (1.33), 4.218 (0.45), 4.231 (0.78), 4.244 |
| | (0.45), 4.317 (0.63), 5.760 (16.00), 6.858 (1.16), 6.870 (1.16), 6.967 (0.72), |
| | 6.988 (0.73), 6.994 (1.08), 6.999 (0.91), 7.115 (0.57), 7.120 (0.51), 7.148 |
| | (0.50), 7.152 (0.47), 7.200 (0.50), 8.209 (1.14), 8.221 (1.08), 11.460 (0.56), |
| | 11.466 (0.55). |
| 115 | 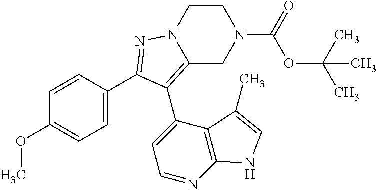 [SMILES: COc1ccc(-c2nn3c(c2-c2ccnc4[nH]cc(C)c24)CN(C(=O)OC(C)(C)C)CC3)cc1] |
| | tert-butyl 2-(4-methoxyphenyl)-3-(3-methyl-1H-pyrrolo[2,3-b]pyridin-4-yl)- |
| | 6,7-dihydropyrazolo[1,5-a]pyrazine-5(4H)-carboxylate |
| | Produced from tert-butyl 2-bromo-3-(3-methyl-1H-pyrrolo[2,3-b]pyridin-4-yl)- |
| | 6,7-dihydropyrazolo[1,5-a]pyrazine-5(4H)-carboxylate (Intermediate 94) and 4- |
| | methoxyphenylboronic acid. |
| | LC-MS (Method 1): Rₜ = 1.06 min; m/z = 460 [M + H]⁺ |
| | ¹H-NMR (400 MHz, DMSO-d6) δ [ppm]: 1.154 (0.61), 1.172 (1.31), 1.190 (0.69), |
| | 1.378 (2.15), 1.423 (0.77), 1.736 (2.24), 1.988 (2.38), 2.518 (0.80), 2.523 (0.51), |
| | 3.653 (16.00), 4.017 (0.55), 4.034 (0.53), 4.207 (0.85), 4.220 (1.48), 4.233 |
| | (0.85), 4.303 (1.11), 5.760 (3.42), 6.732 (3.43), 6.737 (1.00), 6.749 (1.12), 6.754 |
| | (3.54), 6.831 (1.83), 6.843 (1.83), 7.176 (0.92), 7.234 (0.44), 7.241 (3.80), 7.247 |
| | (1.08), 7.258 (1.04), 7.264 (3.15), 8.186 (2.14), 8.198 (1.97), 11.423 (1.11), |
| | 11.428 (1.07). |
| 116 | 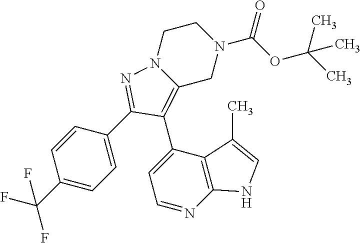 [SMILES: Cc1c[nH]c2nccc(-c3c(-c4ccc(C(F)(F)F)cc4)nn4c3CN(C(=O)OC(C)(C)C)CC4)c12] |
| | tert-butyl 3-(3-methyl-1H-pyrrolo[2,3-b]pyridin-4-yl)-2-[4- |
| | (trifluoromethyl)phenyl]-6,7-dihydropyrazolo[1,5-a]pyrazine-5(4H)- |
| | carboxylate |
| | Produced from tert-butyl 2-bromo-3-(3-methyl-1H-pyrrolo[2,3-b]pyridin-4-yl)- |
| | 6,7-dihydropyrazolo[1,5-a]pyrazine-5(4H)-carboxylate (Intermediate 94) and |
| | [4-(trifluoromethyl)phenyl]boronic acid |
| | LC-MS (Method 1): Rₜ = 1.29 min; m/z = 498 [M + H]⁺ |
| | ¹H-NMR (400 MHz, DMSO-d6) δ [ppm]: 1.066 (0.51), 1.154 (1.69), 1.171 (3.38), |
| | 1.189 (1.64), 1.232 (0.49), 1.259 (0.42), 1.381 (4.81), 1.428 (2.15), 1.691 (0.50), |
| | 1.725 (4.12), 1.987 (5.75), 2.518 (2.21), 2.523 (1.52), 3.942 (0.51), 3.999 (0.65), |
| | 4.017 (1.40), 4.034 (1.32), 4.052 (0.44), 4.273 (1.88), 4.286 (3.16), 4.298 (1.87), |
| | 4.334 (2.15), 4.374 (0.50), 5.760 (16.00), 6.872 (3.39), 6.884 (3.29), 7.209 |
| | (1.92), 7.523 (2.67), 7.544 (6.78), 7.565 (7.54), 7.587 (2.53), 8.217 (4.30), 8.228 |
| | (4.04), 11.494 (2.35), 11.499 (2.23). |
| 117 | 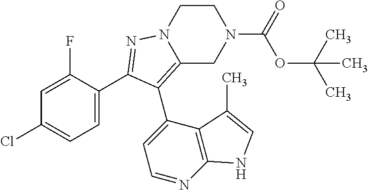 [SMILES: Cc1c[nH]c2nccc(-c3c(-c4ccc(Cl)cc4F)nn4c3CN(C(=O)OC(C)(C)C)CC4)c12] |
| | tert-butyl 2-(4-chloro-2-fluorophenyl)-3-(3-methyl-1H-pyrrolo[2,3- |
| | b]pyridin-4-yl)-6,7-dihydropyrazolo[1,5-a]pyrazine-5(4H)-carboxylate |
| | Produced from tert-butyl 2-bromo-3-(3-methyl-1H-pyrrolo[2,3-b]pyridin-4-yl)- |
| | 6,7-dihydropyrazolo[1,5-a]pyrazine-5(4H)-carboxylate (Intermediate 94) and (4- |
| | chloro-2-fluorophenyl)boronic acid. |
| | LC-MS (Method 1): Rₜ = 1.18 min; m/z = 482 [M + H]⁺ |
| | ¹H-NMR (400 MHz, DMSO-d6) δ [ppm]: 1.382 (13.61), 1.821 (8.11), 2.318 |
| | (1.21), 2.518 (16.00), 2.523 (10.90), 2.660 (1.26), 3.769 (0.60), 4.007 (0.74), |
| | 4.088 (1.87), 4.270 (6.38), 4.302 (3.81), 4.399 (0.84), 6.692 (2.73), 7.189 (5.92), |
| | 7.194 (4.88), 7.210 (5.22), 7.215 (5.15), 7.306 (6.81), 7.311 (6.20), 7.324 (5.79), |
| | 7.331 (7.12), 7.336 (6.65), 7.344 (8.11), 7.364 (4.32), 7.482 (0.77), 7.497 (0.76), |
| | 7.524 (0.81), 7.552 (0.93), 7.568 (0.68), 7.628 (0.44), 8.105 (3.57), 8.116 (3.35), |
| | 11.400 (5.08). |
| 118 | 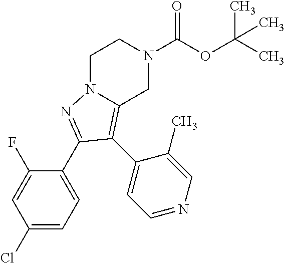 [SMILES: Cc1cnccc1-c1c(-c2ccc(Cl)cc2F)nn2c1CN(C(=O)OC(C)(C)C)CC2] |
| | tert-butyl 2-(4-chloro-2-fluorophenyl)-3-(3-methylpyridin-4-yl)-6,7- |
| | dihydropyrazolo[1,5-a]pyrazine-5(4H)-carboxylate |
| | Produced from tert-butyl 2-bromo-3-(3-methylpyridin-4-yl)-6,7- |
| | dihydropyrazolo[1,5-a]pyrazine-5(4H)-carboxylate (Intermediate 95) and (4- |
| | chloro-2-fluorophenyl)boronic acid. |
| | LC-MS (Method 3): Rₜ = 1.12 min; m/z = 444 |
| 119 | 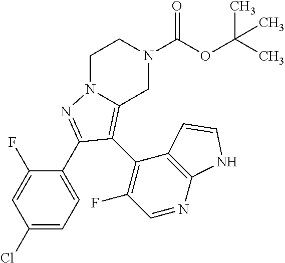 [SMILES: CC(C)(C)OC(=O)N1CCn2nc(-c3ccc(Cl)cc3F)c(-c3c(F)cnc4[nH]ccc34)c2C1] |
| | tert-butyl 2-(4-chloro-2-fluorophenyl)-3-(5-fluoro-1H-pyrrolo[2,3-b]pyridin- |
| | 4-yl)-6,7-dihydropyrazolo[1,5-a]pyrazine-5(4H)-carboxylate |
| | Produced from tert-butyl 2-bromo-3-(5-fluoro-1H-pyrrolo[2,3-b]pyridin-4-yl)-6,7- |
| | dihydropyrazolo[1,5-a]pyrazine-5(4H)-carboxylate (Intermediate 96) and (4- |
| | chloro-2-fluorophenyl)boronic acid |
| | LC-MS (Method 1): Rₜ = 1.25 min; m/z = 486 [M + H]⁺ |
| | ¹H-NMR (400 MHz, DMSO-d6) δ [ppm]: 0.776 (0.47), 0.794 (1.14), 0.813 |
| | (0.71), 0.819 (0.49), 0.836 (0.68), 0.844 (0.64), 0.852 (0.62), 0.862 (0.82), |
| | 1.006 (0.97), 1.034 (0.75), 1.065 (1.26), 1.084 (2.13), 1.154 (4.36), 1.172 |
| | (8.50), 1.190 (4.19), 1.237 (1.11), 1.259 (3.41), 1.406 (13.54), 1.440 (1.18), |
| | 1.987 (16.00), 2.084 (7.33), 2.518 (6.04), 2.523 (4.17), 3.940 (0.84), 3.966 |
| | (0.98), 3.999 (1.54), 4.017 (3.85), 4.035 (3.74), 4.053 (1.21), 4.194 (0.81), |
| | 4.208 (1.44), 4.222 (0.71), 4.286 (1.34), 4.299 (2.35), 4.312 (1.19), 4.360 |
| | (0.59), 4.402 (0.92), 4.463 (0.62), 4.504 (1.45), 5.759 (0.72), 5.979 (1.25), |
| | 6.307 (1.05), 6.311 (1.13), 6.315 (1.11), 6.320 (1.02), 7.258 (1.48), 7.263 |
| | (1.88), 7.283 (6.09), 7.306 (2.13), 7.312 (1.67), 7.435 (0.77), 7.448 (2.19), |
| | 7.455 (3.74), 7.462 (2.11), 7.475 (0.68), 7.624 (0.95), 7.631 (1.29), 7.639 |
| | (0.90), 8.193 (3.79), 8.199 (3.59), 8.298 (1.73), 8.304 (1.69), 10.515 (0.44), |
| | 10.636 (0.40), 10.852 (1.32), 11.822 (1.76), 11.979 (0.68). |
| 120 | 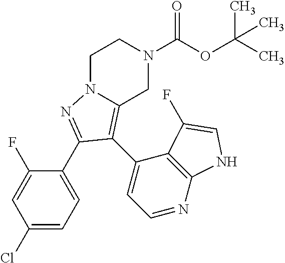 [SMILES: CC(C)(C)OC(=O)N1CCn2nc(-c3ccc(Cl)cc3F)c(-c3ccnc4[nH]cc(F)c34)c2C1] |
| | tert-butyl 2-(4-chloro-2-fluorophenyl)-3-(3-fluoro-1H-pyrrolo[2,3-b]pyridin- |
| | 4-yl)-6,7-dihydropyrazolo[1,5-a]pyrazine-5(4H)-carboxylate |
| | Produced from tert-butyl 2-bromo-3-(3-fluoro-1H-pyrrolo[2,3-b]pyridin-4-yl)-6,7- |
| | dihydropyrazolo[1,5-a]pyrazine-5(4H)-carboxylate (Intermediate 97) and (4- |
| | chloro-2-fluorophenyl)boronic acid |
| | LC-MS (Method 1): Rₜ = 1.22 min; m/z = 486 |
| 121 | 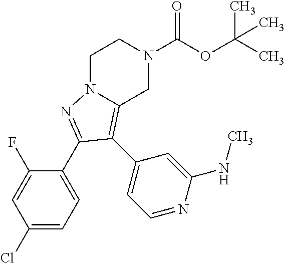 [SMILES: CNc1cc(-c2c(-c3ccc(Cl)cc3F)nn3c2CN(C(=O)OC(C)(C)C)CC3)ccn1] |
| | tert-butyl 2-(4-chloro-2-fluorophenyl)-3-[2-(methylamino)pyridin-4-yl]-6,7- |
| | dihydropyrazolo[1,5-a]pyrazine-5(4H)-carboxylate |
| | Produced from tert-butyl 2-bromo-3-[2-(methylamino)pyridin-4-yl]-6,7- |
| | dihydropyrazolo[1,5-a]pyrazine-5(4H)-carboxylate (Intermediate 98) and (4- |
| | chloro-2-fluorophenyl)boronic acid |
| | LC-MS (Method 1): Rₜ = 0.90 min; m/z = 558 [M + H]⁺ |
| | ¹H-NMR (400 MHz, DMSO-d6) δ [ppm]: 1.427 (15.83), 1.434 (16.00), 2.327 |
| | (1.20), 2.670 (4.81), 2.680 (3.94), 3.801 (0.85), 3.901 (1.87), 4.080 (0.68), |
| | 4.092 (1.03), 4.212 (2.06), 4.469 (1.56), 4.702 (2.55), 6.105 (1.34), 6.137 |
| | (1.04), 6.147 (1.00), 6.454 (0.48), 7.348 (0.95), 7.368 (1.32), 7.445 (1.96), |
| | 7.466 (1.98), 7.485 (0.86), 7.870 (1.44), 7.883 (1.40). |
| 12 123 | 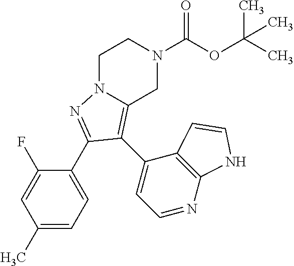 [SMILES: Cc1ccc(-c2nn3c(c2-c2ccnc4[nH]ccc24)CN(C(=O)OC(C)(C)C)CC3)c(F)c1] |
| | tert-butyl 2-(2-fluoro-4-methylphenyl)-3-(1H-pyrrolo[2,3-b]pyridin-4-yl)- |
| | 6,7-dihydropyrazolo[1,5-a]pyrazine-5(4H)-carboxylate |
| | LC-MS (Method 3): Rt = 1.13 min; MS (ESIpos): m/z = 448 [M + H]⁺ |
| | Produced from tert-butyl 2-bromo-3-(1H-pyrrolo[2,3-b]pyridin-4-yl)-6,7- |
| | dihydropyrazolo[1,5-a]pyrazine-5(4H)-carboxylate (Intermediate 151) and |
| | (2-fluoro-4-methylphenyl)boronic acid |
| 124 | 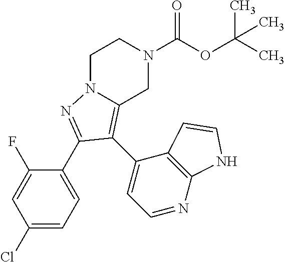 [SMILES: CC(C)(C)OC(=O)N1CCn2nc(-c3ccc(Cl)cc3F)c(-c3ccnc4[nH]ccc34)c2C1] |
| | tert-butyl 2-(4-chloro-2-fluorophenyl)-3-(1H-pyrrolo[2,3-b]pyridin-4-yl)- |
| | 6,7-dihydropyrazolo[1,5-a]pyrazine-5(4H)-carboxylate |
| | Produced from tert-butyl 2-bromo-3-(1H-pyrrolo[2,3-b]pyridin-4-yl)-6,7- |
| | dihydropyrazolo[1,5-a]pyrazine-5(4H)-carboxylate (Intermediate 151) and (4- |
| | chloro-2-fluorophenyl)boronic acid |
| | LC-MS (Method 3): Rt = 1.21 min; MS (ESIpos): m/z = 468 [M + H]⁺ |
| 125 | 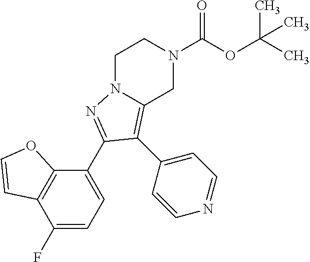 [SMILES: CC(C)(C)OC(=O)N1CCn2nc(-c3ccc(F)c4ccoc34)c(-c3ccncc3)c2C1] |
| | tert-butyl 2-(4-fluoro-1-benzofuran-7-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and 4-fluoro-7-(4,4,5,5- |
| | tetramethyl-1,3,2-dioxaborolan-2-yl)-1-benzofuran. |
| | LC-MS (Method 2): Rt = 1.24 min; MS (ESIpos): m/z = 435 [M + H]⁺ |
| | ¹H-NMR (400 MHz, DMSO-d6) δ [ppm]: 1.154 (0.97), 1.172 (1.90), 1.189 |
| | (0.91), 1.291 (0.90), 1.432 (4.81), 1.481 (1.02), 1.987 (3.20), 2.518 (0.68), |
| | 2.523 (0.44), 2.563 (0.51), 3.933 (0.63), 3.947 (0.41), 4.017 (0.67), 4.035 |
| | (0.69), 4.250 (0.51), 4.265 (0.75), 4.278 (0.44), 4.792 (1.01), 5.759 (16.00), |
| | 6.992 (1.43), 6.996 (0.92), 7.003 (0.91), 7.007 (1.51), 7.031 (0.43), 7.037 |
| | (0.45), 7.056 (2.12), 7.061 (2.14), 7.132 (0.56), 7.153 (0.93), 7.156 (0.61), |
| | 7.176 (0.76), 7.206 (0.43), 7.287 (0.48), 7.300 (0.48), 7.308 (0.40), 7.830 |
| | (1.11), 7.835 (1.36), 8.219 (0.62), 8.400 (1.53), 8.404 (1.15), 8.411 (0.97), |
| | 8.415 (1.60), 8.421 (0.68). |
| 126 | 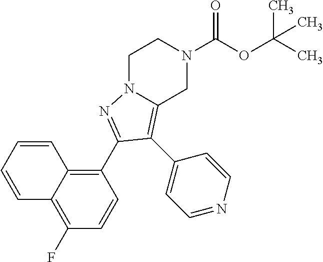 [SMILES: CC(C)(C)OC(=O)N1CCn2nc(-c3ccc(F)c4ccccc34)c(-c3ccncc3)c2C1] |
| | tert-butyl 2-(4-fluoronaphthalen-1-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and 2-(4-fluoro-1-naphthyl)- |
| | 4,4,5,5-tetramethyl-1,3,2-dioxaborolane. |
| | LC-MS (Method 2): Rt = 1.33 min; MS (ESIpos): m/z = 445 [M + H]⁺ |
| | ¹H-NMR (400 MHz, DMSO-d6) δ [ppm]: 1.066 (4.24), 1.154 (3.59), 1.172 |
| | (7.69), 1.189 (3.89), 1.405 (6.20), 1.447 (15.40), 1.491 (0.51), 1.987 (13.78), |
| | 2.518 (1.20), 2.523 (0.79), 3.565 (0.77), 3.841 (0.46), 3.854 (0.81), 3.868 |
| | (0.54), 3.942 (0.77), 3.953 (1.42), 3.966 (2.48), 3.980 (1.62), 3.999 (1.02), |
| | 4.016 (2.82), 4.035 (2.80), 4.053 (0.93), 4.145 (0.67), 4.159 (0.98), 4.172 |
| | (0.54), 4.281 (1.80), 4.294 (2.78), 4.308 (1.53), 4.698 (1.68), 4.851 (4.72), |
| | 5.759 (16.00), 6.903 (6.01), 6.907 (3.32), 6.915 (3.48), 6.919 (5.89), 7.346 |
| | (0.71), 7.365 (2.78), 7.377 (2.06), 7.390 (3.76), 7.423 (1.93), 7.427 (1.16), |
| | 7.434 (1.16), 7.438 (1.98), 7.473 (0.98), 7.476 (1.01), 7.490 (1.38), 7.494 |
| | (1.89), 7.498 (1.22), 7.512 (1.43), 7.515 (1.37), 7.595 (1.29), 7.598 (1.30), |
| | 7.615 (1.91), 7.618 (1.48), 7.633 (1.07), 7.636 (1.01), 7.816 (1.52), 7.838 |
| | (1.31), 8.093 (2.16), 8.114 (1.97), 8.328 (6.11), 8.332 (3.51), 8.339 (3.27), |
| | 8.343 (5.88), 8.639 (1.98), 8.644 (1.08), 8.651 (1.11), 8.655 (1.89). |
| 127 | 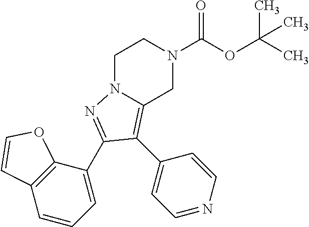 [SMILES: CC(C)(C)OC(=O)N1CCn2nc(-c3cccc4ccoc34)c(-c3ccncc3)c2C1] |
| | tert-butyl 2-(1-benzofuran-7-yl)-3-(pyridin-4-yl)-6,7-dihydropyrazolo[1,5- |
| | a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and |
| | 7-(4,4,5,5-tetramethyl-1,3,2-dioxaborolan-2-yl)-1-benzofuran. |
| | LC-MS (Method 2): Rt = 1.21 min; MS (ESIpos): m/z = 417 [M + H]⁺ |
| | ¹H-NMR (400 MHz, DMSO-d6) δ [ppm]: 1.066 (1.48), 1.154 (0.87), 1.172 |
| | (1.74), 1.190 (0.89), 1.406 (5.23), 1.435 (16.00), 1.483 (0.46), 1.988 (2.95), |
| | 2.518 (2.07), 2.523 (1.34), 3.854 (0.63), 3.868 (0.40), 3.924 (1.53), 3.937 |
| | (2.69), 3.951 (1.73), 4.017 (0.66), 4.035 (0.62), 4.146 (0.48), 4.160 (0.75), |
| | 4.255 (1.98), 4.269 (3.04), 4.283 (1.64), 4.698 (1.26), 4.797 (4.19), 5.760 |
| | (14.34), 6.931 (6.29), 6.936 (6.38), 6.984 (6.33), 6.988 (3.77), 6.995 (3.88), |
| | 6.999 (6.28), 7.279 (5.67), 7.291 (5.47), 7.423 (1.37), 7.427 (0.86), 7.434 |
| | (0.88), 7.439 (1.41), 7.662 (0.46), 7.671 (2.68), 7.683 (3.96), 7.694 (2.26), |
| | 7.748 (5.06), 7.753 (4.83), 8.382 (6.65), 8.387 (3.74), 8.394 (3.77), 8.398 |
| | (6.11), 8.640 (1.47), 8.644 (0.82), 8.651 (0.83), 8.655 (1.39). |
| 128 | 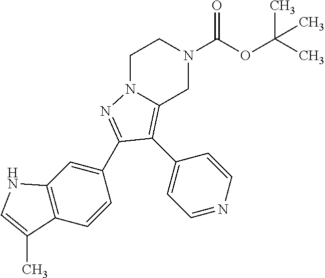 [SMILES: Cc1c[nH]c2cc(-c3nn4c(c3-c3ccncc3)CN(C(=O)OC(C)(C)C)CC4)ccc12] |
| | tert-butyl 2-(3-methyl-3H-indol-6-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and 3-methyl-6-(4,4,5,5- |
| | tetramethyl-1,3,2-dioxaborolan-2-yl)-3H-indole. |
| | LC-MS (Method 2): Rt = 1.22 min; MS (ESIneg): m/z = 428 [M − H]⁻ |
| | ¹H-NMR (400 MHz, DMSO-d6) δ [ppm]: 1.066 (0.96), 1.154 (4.18), 1.172 |
| | (8.99), 1.190 (4.61), 1.407 (2.72), 1.423 (6.76), 1.988 (16.00), 2.236 (6.22), |
| | 2.238 (6.55), 2.518 (0.52), 3.891 (0.68), 3.904 (1.20), 3.917 (0.77), 3.999 |
| | (1.22), 4.017 (3.67), 4.035 (3.57), 4.053 (1.13), 4.201 (0.91), 4.215 (1.40), |
| | 4.229 (0.76), 4.684 (2.17), 4.698 (0.68), 5.759 (3.78), 6.994 (0.74), 6.997 |
| | (0.74), 7.015 (0.79), 7.018 (0.80), 7.105 (1.09), 7.107 (1.37), 7.110 (1.35), |
| | 7.113 (1.05), 7.136 (2.79), 7.140 (1.71), 7.147 (1.72), 7.151 (2.80), 7.299 |
| | (1.64), 7.410 (1.47), 7.423 (0.56), 7.430 (1.38), 7.438 (0.52), 8.489 (2.82), |
| | 8.493 (1.61), 8.500 (1.54), 8.505 (2.63), 8.640 (0.45), 8.655 (0.44), 10.747 |
| | (0.97), 10.752 (0.96). |
| 129 | 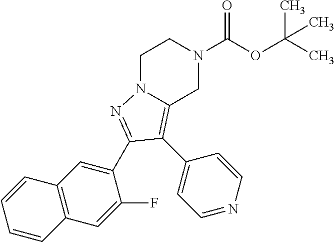 [SMILES: CC(C)(C)OC(=O)N1CCn2nc(-c3cc4ccccc4cc3F)c(-c3ccncc3)c2C1] |
| | tert-butyl 2-(3-fluoronaphthalen-2-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (3-fluoro-2- |
| | naphthyl)boronic acid. |
| | LC-MS (Method 2): Rt = 1.33 min; MS (ESIpos): m/z = 445 [M + H]⁺ |
| | ¹H-NMR (400 MHz, DMSO-d6) δ [ppm]: 1.066 (4.61), 1.154 (3.79), 1.172 |
| | (7.85), 1.189 (3.91), 1.406 (2.21), 1.436 (16.00), 1.486 (0.47), 1.987 (13.75), |
| | 2.518 (1.00), 2.523 (0.66), 3.565 (0.80), 3.931 (1.55), 3.942 (3.06), 3.958 |
| | (1.75), 3.999 (0.97), 4.017 (2.90), 4.035 (2.93), 4.053 (0.96), 4.269 (2.01), |
| | 4.283 (3.12), 4.296 (1.70), 4.806 (4.35), 5.759 (11.17), 7.049 (6.80), 7.053 |
| | (3.87), 7.060 (3.99), 7.064 (6.52), 7.423 (0.44), 7.438 (0.44), 7.511 (0.88), |
| | 7.514 (0.93), 7.531 (2.09), 7.548 (1.68), 7.551 (1.55), 7.573 (1.44), 7.591 |
| | (1.91), 7.608 (0.92), 7.697 (2.57), 7.724 (2.56), 7.910 (2.26), 7.930 (2.07), |
| | 8.011 (2.13), 8.032 (1.92), 8.135 (2.85), 8.154 (2.81), 8.447 (6.79), 8.451 |
| | (3.95), 8.458 (3.92), 8.463 (6.41), 8.640 (0.44), 8.655 (0.43). |
| 130 | 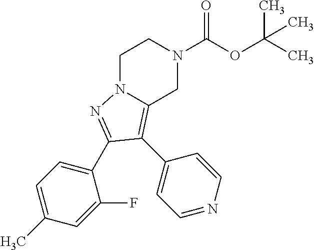 [SMILES: Cc1ccc(-c2nn3c(c2-c2ccncc2)CN(C(=O)OC(C)(C)C)CC3)c(F)c1] |
| | tert-butyl 2-(2-fluoro-4-methylphenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (2-fluoro-4- |
| | methylphenyl)boronic acid. |
| | LC-MS (Method 2): Rt = 1.18 min; MS (ESIpos): m/z = 409 [M + H]⁺ |
| 131 | 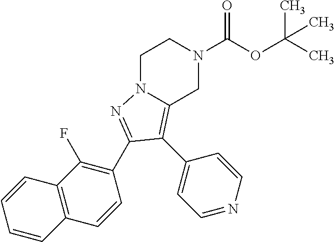 [SMILES: CC(C)(C)OC(=O)N1CCn2nc(-c3ccc4ccccc4c3F)c(-c3ccncc3)c2C1] |
| | tert-butyl 2-(1-fluoronaphthalen-2-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (1-fluoro-2- |
| | naphthyl)boronic acid |
| | LC-MS (Method 2): Rt = 1.31 min; MS (ESIpos): m/z = 445 [M + H]⁺ |
| | 1H-NMR (400 MHz, DMSO-d6) delta [ppm]: 1.171 (0.25), 1.311 (0.19), 1.406 |
| | (0.30), 1.434 (1.71), 1.485 (0.18), 1.987 (0.41), 2.518 (0.37), 2.523 (0.25), |
| | 3.943 (0.26), 3.957 (0.17), 4.269 (0.20), 4.283 (0.31), 4.296 (0.18), 4.801 |
| | (0.44), 5.758 (16.00), 7.067 (0.67), 7.071 (0.38), 7.078 (0.39), 7.082 (0.64), |
| | 7.562 (0.18), 7.565 (0.19), 7.583 (0.17), 7.604 (0.22), 7.608 (0.22), 7.625 |
| | (0.38), 7.630 (0.36), 7.646 (0.23), 7.649 (0.25), 7.831 (0.35), 7.853 (0.31), |
| | 7.925 (0.22), 7.945 (0.19), 8.028 (0.21), 8.047 (0.21), 8.445 (0.73), 8.450 |
| | (0.51), 8.457 (0.40), 8.461 (0.73), 8.465 (0.17). |
| 132 | 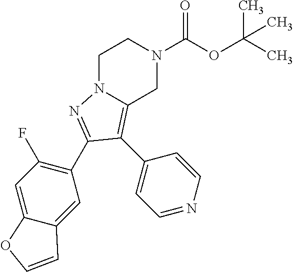 [SMILES: CC(C)(C)OC(=O)N1CCn2nc(-c3cc4ccoc4cc3F)c(-c3ccncc3)c2C1] |
| | tert-butyl 2-(6-fluoro-1-benzofuran-5-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and 6-fluoro-5-(4,4,5,5- |
| | tetramethyl-1,3,2-dioxaborolan-2-yl)-1-benzofuran |
| | LC-MS (Method 2): Rt = 1.22 min; MS (ESIpos): m/z = 435 [M + H]⁺ |
| | 1H-NMR (400 MHz, DMSO-d6) delta [ppm]: 0.864 (0.02), 0.907 (0.05), 1.026 |
| | (0.02), 1.065 (16.00), 1.153 (0.14), 1.171 (0.25), 1.189 (0.12), 1.220 (0.06), |
| | 1.230 (0.04), 1.309 (0.05), 1.316 (0.06), 1.429 (0.71), 1.478 (0.11), 1.905 |
| | (0.02), 1.986 (0.47), 2.518 (0.15), 2.523 (0.10), 3.565 (0.03), 3.915 (0.06), |
| | 3.929 (0.12), 3.943 (2.79), 3.998 (0.04), 4.016 (0.11), 4.034 (0.11), 4.052 |
| | (0.04), 4.159 (0.02), 4.235 (0.08), 4.249 (0.13), 4.262 (0.07), 4.698 (0.01), |
| | 4.786 (0.17), 5.529 (0.02), 5.758 (2.91), 5.982 (0.01), 7.013 (0.17), 7.015 |
| | (0.19), 7.019 (0.21), 7.021 (0.37), 7.026 (0.16), 7.033 (0.15), 7.037 (0.24), |
| | 7.239 (0.04), 7.254 (0.03), 7.423 (0.02), 7.438 (0.01), 7.565 (0.12), 7.589 |
| | (0.11), 7.732 (0.04), 7.738 (0.16), 7.750 (0.05), 7.756 (0.15), 8.060 (0.28), |
| | 8.066 (0.27), 8.088 (0.02), 8.442 (0.24), 8.445 (0.15), 8.453 (0.16), 8.457 |
| | (0.23), 8.463 (0.06), 8.639 (0.01), 8.654 (0.02). |
| 133 | 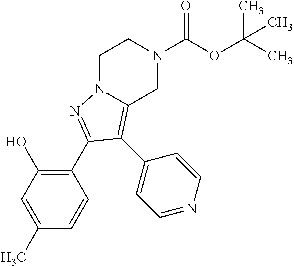 [SMILES: Cc1ccc(-c2nn3c(c2-c2ccncc2)CN(C(=O)OC(C)(C)C)CC3)c(O)c1] |
| | tert-butyl 2-(2-hydroxy-4-methylphenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (2-hydroxy-4- |
| | methylphenyl)boronic acid |
| | LC-MS (Method 2): Rt = 1.25 min; MS (ESIpos): m/z = 407 [M + H]⁺ |
| 134 | 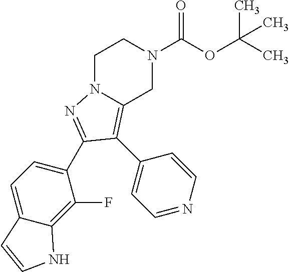 [SMILES: CC(C)(C)OC(=O)N1CCn2nc(-c3ccc4cc[nH]c4c3F)c(-c3ccncc3)c2C1] |
| | tert-butyl 2-(7-fluoro-1H-indol-6-yl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and 7-fluoro-6-(4,4,5,5- |
| | tetramethyl-1,3,2-dioxaborolan-2-yl)-1H-indole |
| | LC-MS (Method 2): Rt = 1.15 min; MS (ESIpos): m/z = 434 [M + H]⁺ |
| | 1H-NMR (400 MHz, DMSO-d6) delta [ppm]: 0.907 (0.05), 1.065 (16.00), |
| | 1.154 (0.91), 1.171 (1.94), 1.189 (0.99), 1.220 (0.06), 1.230 (0.07), 1.258 |
| | (0.07), 1.318 (0.23), 1.430 (0.96), 1.480 (0.16), 1.898 (0.04), 1.986 (3.48), |
| | 2.523 (0.08), 3.397 (0.03), 3.928 (0.15), 3.945 (2.19), 3.998 (0.28), 4.016 |
| | (0.82), 4.034 (0.80), 4.052 (0.25), 4.088 (0.08), 4.232 (0.12), 4.247 (0.17), |
| | 4.259 (0.10), 4.779 (0.22), 6.532 (0.16), 6.537 (0.15), 6.544 (0.08), 7.023 |
| | (0.10), 7.039 (0.13), 7.046 (0.37), 7.050 (0.23), 7.058 (0.26), 7.062 (0.36), |
| | 7.201 (0.03), 7.263 (0.07), 7.278 (0.06), 7.397 (0.39), 7.405 (0.28), 7.411 |
| | (0.19), 7.416 (0.24), 8.432 (0.34), 8.437 (0.28), 8.444 (0.21), 8.447 (0.36), |
| | 8.452 (0.13), 8.527 (0.03), 9.783 (0.03), 11.580 (0.15). |
| 135 | 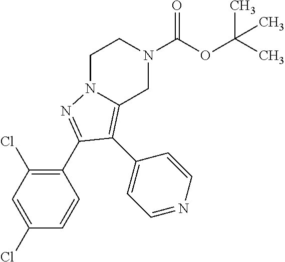 [SMILES: CC(C)(C)OC(=O)N1CCn2nc(-c3ccc(Cl)cc3Cl)c(-c3ccncc3)c2C1] |
| | tert-butyl 2-(2,4-dichlorophenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (2,4- |
| | dichlorophenyl)boronic acid |
| | LC-MS (Method 2): Rt = 1.33 min; MS (ESIpos): m/z = 445 [M + H]⁺ |
| | ¹H-NMR (400 MHz, DMSO-d6) δ [ppm]: 1.154 (4.47), 1.172 (9.29), 1.189 |
| | (4.56), 1.231 (0.50), 1.425 (15.03), 1.474 (2.35), 1.987 (16.00), 2.518 (2.10), |
| | 2.523 (1.32), 3.367 (0.69), 3.374 (0.44), 3.911 (1.27), 3.924 (2.30), 3.937 |
| | (1.52), 3.999 (1.17), 4.016 (3.50), 4.034 (3.40), 4.052 (1.08), 4.224 (1.77), |
| | 4.238 (2.68), 4.251 (1.51), 4.797 (4.28), 6.966 (5.57), 6.970 (3.19), 6.977 |
| | (3.32), 6.981 (5.42), 7.427 (0.52), 7.476 (1.74), 7.497 (4.64), 7.515 (4.02), |
| | 7.520 (4.00), 7.536 (1.38), 7.541 (1.64), 7.676 (4.23), 7.681 (4.06), 8.462 |
| | (5.57), 8.465 (3.16), 8.473 (3.32), 8.477 (5.23). |
| 136 | 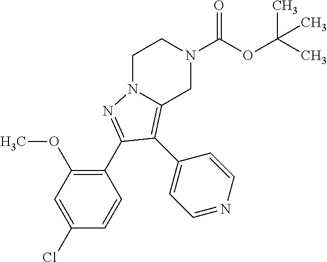 [SMILES: COc1cc(Cl)ccc1-c1nn2c(c1-c1ccncc1)CN(C(=O)OC(C)(C)C)CC2] |
| | tert-butyl 2-(4-chloro-2-methoxyphenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (4-chloro-2- |
| | methoxyphenyl)boronic acid |
| | LC-MS (Method 2): Rt = 1.26 min; MS (ESIpos): m/z = 441 [M + H]⁺ |
| | ¹H-NMR (400 MHz, DMSO-d6) δ [ppm]: 1.154 (3.71), 1.172 (7.73), 1.190 |
| | (3.81), 1.233 (0.45), 1.422 (13.51), 1.470 (1.84), 1.988 (13.56), 2.518 (6.40), |
| | 2.523 (3.99), 3.235 (0.68), 3.280 (16.00), 3.823 (0.40), 3.889 (1.20), 3.902 |
| | (2.16), 3.915 (1.43), 3.999 (1.05), 4.017 (3.07), 4.035 (3.05), 4.053 (0.97), |
| | 4.184 (1.64), 4.198 (2.55), 4.212 (1.37), 4.752 (3.15), 5.759 (13.69), 6.972 |
| | (4.97), 6.975 (2.91), 6.983 (2.99), 6.987 (5.02), 7.055 (2.87), 7.060 (3.95), |
| | 7.074 (2.66), 7.079 (1.72), 7.094 (2.67), 7.100 (2.08), 7.363 (3.51), 7.383 |
| | (3.09), 8.448 (5.01), 8.452 (2.98), 8.460 (3.03), 8.463 (4.83), 8.546 (0.40). |
| 137 | 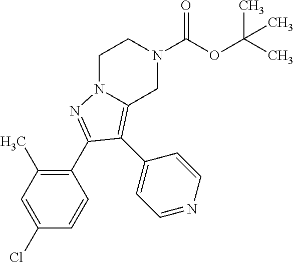 [SMILES: Cc1cc(Cl)ccc1-c1nn2c(c1-c1ccncc1)CN(C(=O)OC(C)(C)C)CC2] |
| | tert-butyl 2-(4-chloro-2-methylphenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (4-chloro-2- |
| | methylphenyl)boronic acid |
| | LC-MS (Method 2): Rt = 1.33 min; MS (ESIpos): m/z = 425 [M + H]⁺ |
| | ¹H-NMR (400 MHz, DMSO-d6) δ [ppm]: 1.154 (0.82), 1.172 (1.76), 1.190 |
| | (0.88), 1.406 (2.33), 1.427 (9.35), 1.475 (0.64), 1.988 (3.42), 2.000 (7.86), |
| | 2.523 (1.72), 3.904 (0.79), 3.917 (1.40), 3.930 (0.91), 4.017 (0.72), 4.035 |
| | (0.69), 4.208 (1.06), 4.222 (1.68), 4.236 (0.88), 4.698 (0.45), 4.788 (2.67), |
| | 5.759 (16.00), 6.956 (3.06), 6.960 (1.86), 6.968 (1.85), 6.972 (3.09), 7.168 |
| | (1.15). 7.188 (1.68), 7.265 (0.99), 7.270 (1.05), 7.286 (0.64), 7.291 (0.73), |
| | 7.360 (1.65), 7.365 (1.45), 7.423 (0.45), 7.438 (0.50), 8.448 (3.11), 8.451 |
| | (1.86), 8.459 (1.82), 8.463 (2.97), 8.640 (0.46), 8.655 (0.43). |
| 138 | 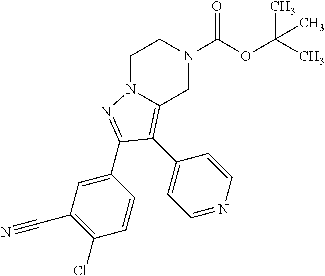 [SMILES: CC(C)(C)OC(=O)N1CCn2nc(-c3ccc(Cl)c(C#N)c3)c(-c3ccncc3)c2C1] |
| | tert-butyl 2-(4-chloro-3-cyanophenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (4-chloro-3- |
| | cyanophenyl)boronic acid |
| | LC-MS (Method 2): Rt = 1.26 min; MS (ESIpos): m/z = 436 [M + H]⁺ |
| | ¹H-NMR (400 MHz, DMSO-d6) δ [ppm]: 1.154 (4.44), 1.172 (9.10), 1.190 |
| | (4.43), 1.232 (0.60), 1.415 (8.19), 1.435 (3.04), 1.465 (1.01), 1.988 (16.00), |
| | 2.518 (3.25), 2.523 (2.16), 3.885 (0.73), 3.899 (1.28), 3.913 (0.85), 3.999 |
| | (1.16), 4.017 (3.51), 4.035 (3.39), 4.053 (1.09), 4.159 (0.44), 4.227 (1.00), |
| | 4.241 (1.53), 4.255 (0.90), 4.674 (2.63), 4.698 (0.49), 7.188 (3.11), 7.192 |
| | (1.83), 7.199 (1.87), 7.203 (3.12), 7.423 (0.54), 7.438 (0.51), 7.593 (0.76), |
| | 7.598 (0.74), 7.614 (0.87), 7.619 (0.91), 7.729 (2.48), 7.750 (1.82), 7.880 |
| | (0.40), 7.886 (1.69), 7.891 (1.64), 8.572 (3.24), 8.575 (1.78), 8.582 (1.79), |
| | 8.587 (3.08), 8.640 (0.53), 8.655 (0.51). |
| 139 | 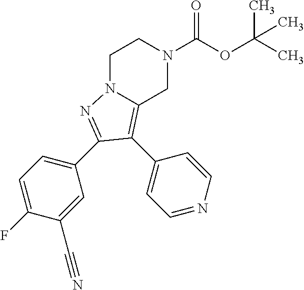 [SMILES: CC(C)(C)OC(=O)N1CCn2nc(-c3ccc(F)c(C#N)c3)c(-c3ccncc3)c2C1] |
| | tert-butyl 2-(3-cyano-4-fluorophenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (3-cyano-4- |
| | fluorophenyl)boronic acid |
| | LC-MS (Method 2): Rt = 1.20 min; MS (ESIpos): m/z = 420 [M + H]⁺ |
| 140 | 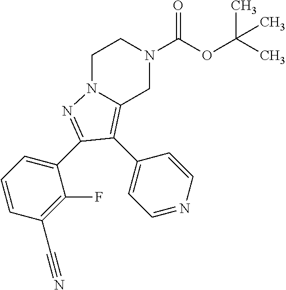 [SMILES: CC(C)(C)OC(=O)N1CCn2nc(-c3cccc(C#N)c3F)c(-c3ccncc3)c2C1] |
| | tert-butyl 2-(3-cyano-2-fluorophenyl)-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7-dihydropyrazolo[1,5- |
| | a]pyrazine-5(4H)-carboxylate (Intermediate 68) and (3-cyano-2- |
| | fluorophenyl)boronic acid |
| | LC-MS (Method 2): Rt = 1.15 min; MS (ESIpos): m/z = 420 [M + H]⁺ |
| 141 | 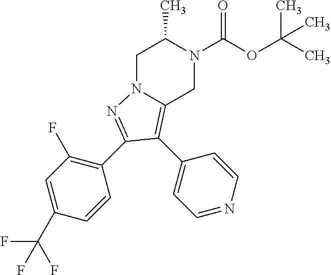 [SMILES: C[C@H]1Cn2nc(-c3ccc(C(F)(F)F)cc3F)c(-c3ccncc3)c2CN1C(=O)OC(C)(C)C] |
| | tert-butyl (6S)-2-[2-fluoro-4-(trifluoromethyl)phenyl]-6-methyl-3-(pyridin- |
| | 4-yl)-6,7-dihydropyrazolo[1,5-a]pyrazine-5(4H)-carboxylate |
| | Produced from tert-butyl (6S)-2-bromo-6-methyl-3-(pyridin-4-yl)-6,7- |
| | dihydropyrazolo[1,5-a]pyrazine-5(4H)-carboxylate (Intermediate 498) and |
| | [2-fluoro-4-(trifluoromethyl)phenyl]boronic acid |
| | LC-MS (Method 2): Rt = 1.38 min; MS (ESIpos): m/z = 477 [M + H]⁺ |
| 142 | 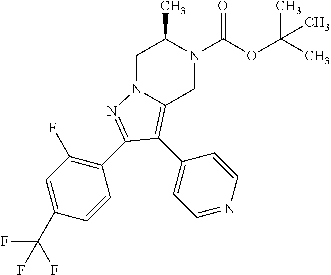 [SMILES: C[C@@H]1Cn2nc(-c3ccc(C(F)(F)F)cc3F)c(-c3ccncc3)c2CN1C(=O)OC(C)(C)C] |
| | tert-butyl (6R)-2-[2-fluoro-4-(trifluoromethyl)phenyl]-6-methyl-3-(pyridin- |
| | 4-yl)-6,7-dihydropyrazolo[1,5-a]pyrazine-5(4H)-carboxylate |
| | Produced from tert-butyl (6R)-2-bromo-6-methyl-3-(pyridin-4-yl)-6,7- |
| | dihydropyrazolo[1,5-a]pyrazine-5(4H)-carboxylate (Intermediate 501) |
| | and [2-fluoro-4-(trifluoromethyl)phenyl]boronic acid |
| | LC-MS (Method 2): Rt = 1.38 min; MS (ESIpos): m/z = 477 [M + H]⁺ |
| 143 | 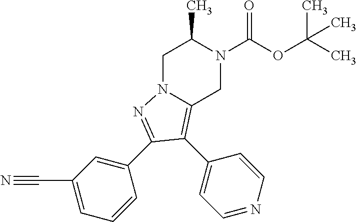 [SMILES: C[C@@H]1Cn2nc(-c3cccc(C#N)c3)c(-c3ccncc3)c2CN1C(=O)OC(C)(C)C] |
| | tert-butyl (6R)-2-(3-cyanophenyl)-6-methyl-3-(pyridin-4-yl)-6,7- |
| | dihydropyrazolo[1,5-a]pyrazine-5(4H)-carboxylate |
| | Produced from tert-butyl (6R)-2-bromo-6-methyl-3-(pyridin-4-yl)-6,7- |
| | dihydropyrazolo[1,5-a]pyrazine-5(4H)-carboxylate (Intermediate 501) |
| | and (3-cyanophenyl)boronic acid |
| | LC-MS (Method 3): Rt = 0.95 min; MS (ESIpos): m/z = 416 [M + H]⁺ |
| | 1H NMR (400 MHz, DMSO-d6, 22 ºC): δ = 8.54-8.59 (m, 2H), 7.81 (dt, |
| | J = 7.6, 1.5 Hz, 1H), 7.77 (t, J = 1.5 Hz, 1H), 7.62-7.66 (m, 1H), 7.53- |
| | 7.59 (m, 1H), 7.21-7.25 (m, 1H), 7.25 (br s, 1H), 4.81 (d, J = 17.2 Hz, |
| | 1H), 4.49 (br d, J = 17.0 Hz, 1H), 4.48 (br s, 1H), 4.27-4.34 (m, 1H), |
| | 4.17-4.23 (m, 1H), 1.42 (s, 9H), 1.18 ppm (d, J = 7.1 Hz, 2H) |
| 144 | 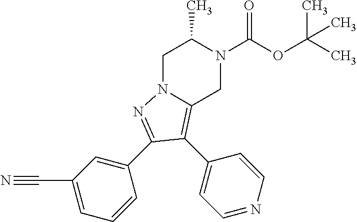 [SMILES: C[C@H]1Cn2nc(-c3cccc(C#N)c3)c(-c3ccncc3)c2CN1C(=O)OC(C)(C)C] |
| | tert-butyl (6S)-2-(3-cyanophenyl)-6-methyl-3-(pyridin-4-yl)-6,7- |
| | dihydropyrazolo[1,5-a]pyrazine-5(4H)-carboxylate |
| | Produced from tert-butyl (6S)-2-bromo-6-methyl-3-(pyridin-4-yl)-6,7- |
| | dihydropyrazolo[1,5-a]pyrazine-5(4H)-carboxylate (Intermediate 498) |
| | and (3-cyanophenyl)boronic acid |
| | LC-MS (Method 3): Rt = 0.95 min; MS (ESIpos): m/z = 416 [M + H]⁺ |
| | 1H NMR (400 MHz, DMSO-d6, 22 ºC): δ = 8.54-8.59 (m, 2H), 7.81 (dt, |
| | J = 7.6, 1.5 Hz, 1H), 7.77 (t, J = 1.5 Hz, 1H), 7.62-7.67 (m, 1H), 7.54- |
| | 7.59 (m, 1H), 7.21-7.25 (m, 2H), 4.81 (d, J = 17.2 Hz, 1H), 4.74 (br d, |
| | J = 3.8 Hz, 1H), 4.44-4.55 (m, 1H), 4.26-4.34 (m, 1H), 4.17-4.24 (m, |
| | 1H), 1.42 (s, 9H), 1.20 ppm (d, J = 6.6 Hz, 3H) |
| 145 | 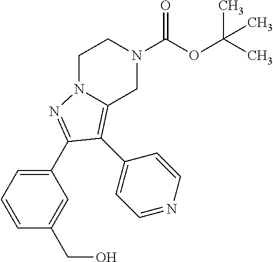 [SMILES: CC(C)(C)OC(=O)N1CCn2nc(-c3cccc(CO)c3)c(-c3ccncc3)c2C1] |
| | tert-butyl 2-[3-(hydroxymethyl)phenyl]-3-(pyridin-4-yl)-6,7- |
| | dihydropyrazolo[1,5-a]pyrazine-5(4H)-carboxylate |
| | Produced from tert-butyl 2-bromo-3-(pyridin-4-yl)-6,7- |
| | dihydropyrazolo[1,5-a]pyrazine-5(4H)-carboxylate (Intermediate |
| | 68) and [3-(hydroxymethyl)phenyl]boronic acid |
| | LC-MS (Method 3): Rt = 0.82 min; MS (ESIpos): m/z = 408 [M + H]⁺ |

The following intermediates were prepared analogous to the preparation of Intermediate 4 starting from the corresponding Boc-protected intermediates mentioned in the table below by reacting with trifluoroacetic acid.

| Inter- | Structure | |
|---|---|---|
| mediate | Name | Analytical Data |
| 146 | 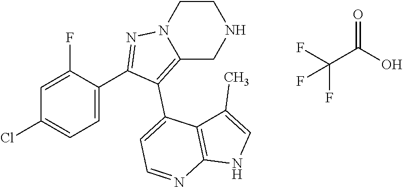 [SMILES: Cc1c[nH]c2nccc(-c3c(-c4ccc(Cl)cc4F)nn4c3CNCC4)c12 ; O=C(O)C(F)(F)F] | Prepared from tert-butyl 2-(4-chloro-2- fluorophenyl)-3-(3-methyl-1H-pyrrolo[2,3- b]pyridin-4-yl)-6,7-dihydropyrazolo[1,5- a]pyrazine-5(4H)-carboxylate (Intermediate 117) LC-MS (Method 2): Rt = 0.95 min; m/z = 382.3 (M + H)+ |
| | trifluoroacetic acid-2-(4-chloro-2- | |
| | fluorophenyl)-3-(3-methyl-1H- | |
| | pyrrolo[2,3-b]pyridin-4-yl)-4,5,6,7- | |
| | tetrahydropyrazolo[1,5-a]pyrazine | |
| | (1/1) | |
| 147 | 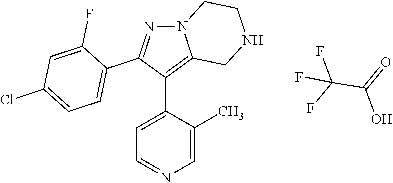 [SMILES: Cc1cnccc1-c1c(-c2ccc(Cl)cc2F)nn2c1CNCC2 ; O=C(O)C(F)(F)F] | Prepared from tert-butyl 2-(4-chloro-2- fluorophenyl)-3-(3-methylpyridin-4-yl)- 6,7-dihydropyrazolo[1,5-a]pyrazine- 5(4H)-carboxylate (Intermediate 118) LC-MS (Method 2): Rt = 0.95 min; m/z = 343.3 (M + H)+ |
| | trifluoroacetic acid-2-(4-chloro-2- | |
| | fluorophenyl)-3-(3-methylpyridin-4-yl)- | |
| | 4,5,6,7-tetrahydropyrazolo[1,5- | |
| | a]pyrazine (1/1) | |
| 148 | 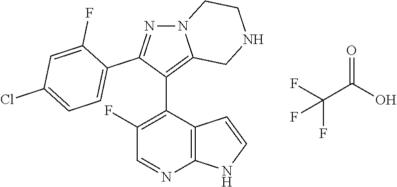 [SMILES: Fc1cc(Cl)ccc1-c1nn2c(c1-c1c(F)cnc3[nH]ccc13)CNCC2 ; O=C(O)C(F)(F)F] | Produced from tert-butyl 2-(4-chloro-2- fluorophenyl)-3-(5-fluoro-1H-pyrrolo[2,3- bjpyridin-4-yl)-6,7-dihydropyrazolo[1,5- a]pyrazine-5(4H)-carboxylate (Intermediate 119) LC-MS (Method 2): Rt = 0.96 min; m/z = 386.3 (M + H)+ |
| | trifluoroacetic acid-2-(4-chloro-2- | |
| | fluorophenyl)-3-(5-fluoro-1H- | |
| | pyrrolo[2,3-b]pyridin-4-yl)-4,5,6,7- | |
| | tetrahydropyrazolo[1,5-a]pyrazine | |
| | (1/1) | |
| 149 | 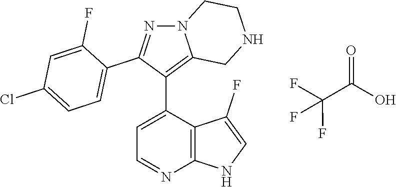 [SMILES: Fc1cc(Cl)ccc1-c1nn2c(c1-c1ccnc3[nH]cc(F)c13)CNCC2 ; O=C(O)C(F)(F)F] | Produced from tert-butyl 2-(4-chloro-2- fluorophenyl)-3-(3-fluoro-1H-pyrrolo[2,3- b]pyridin-4-yl)-6,7-dihydropyrazolo[1,5- a]pyrazine-5(4H)-carboxylate (Intermediate 120) LC-MS (Method 2): Rt = 0.93 min; m/z = 386.3 (M + H)+ |
| | trifluoroacetic acid-2-(4-chloro-2- | |
| | fluorophenyl)-3-(3-fluoro-1H- | |
| | pyrrolo[2,3-b]pyridin-4-yl)-4,5,6,7- | |
| | tetrahydropyrazolo[1,5-a]pyrazine | |
| | (1/1) | |
| 150 | 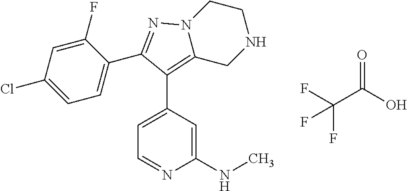 [SMILES: CNc1cc(-c2c(-c3ccc(Cl)cc3F)nn3c2CNCC3)ccn1 ; O=C(O)C(F)(F)F] | Produced from tert-butyl 2-(4-chloro-2- fluorophenyl)-3-[2-(methylamino)pyridin- 4-yl]-6,7-dihydropyrazolo[1,5-a]pyrazine- 5(4H)-carboxylate (Intermediate 121) LC-MS (Method 2): Rt = 0.93 min; m/z = 358.3 (M + H)+ |
| | trifluoroacetic acid-4-[2-(4-chloro-2- | |
| | fluorophenyl)-4,5,6,7- | |
| | tetrahydropyrazolo[1,5-a]pyrazin-3-yl]- | |
| | N-methylpyridin-2-amine (1/1) | |

Intermediate 151

### 3-[3-(3-methyl-1H-pyrrolo[2,3-b]pyridin-4-yl)-4,5,6,7-tetrahydropyrazolo[1,5-a]pyrazin-2-yl]benzonitrile-hydrogen chloride (1/1)

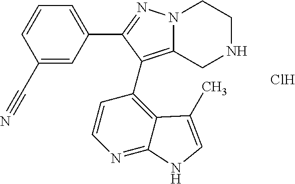 [SMILES: Cc1c[nH]c2nccc(-c3c(-c4cccc(C#N)c4)nn4c3CNCC4)c12 ; Cl]

To a solution of tert-butyl 2-(3-cyanophenyl)-3-(3-methyl-1H-pyrrolo[2,3-b]pyridin-4-yl)-6,7-dihydropyrazolo[1,5-a]pyrazine-5(4H)-carboxylate (31.4 mg, 69.0 μmol, Intermediate 113) in dichloromethane (740 μl) was added hydrochloric acid (260 μl, 4.0 M in dioxane, 1.0 mmol) at 20° C. The resulting mixture was stirred at 20° C. for 2 hours. The mixture was evaporated in vacuum and used in the next step without further purification.

LC-MS (Method 2): Rₜ=0.86 min; MS (ESIpos): m/z=355.3 [M+H]⁺.

The following intermediates were prepared analogous to the preparation of Intermediate 151 starting from the corresponding Boc-protected intermediates by reacting with hydrochloric acid.

| Inter- | Structure | |
|---|---|---|
| mediate | Name | Analytical Data |
| 152 | 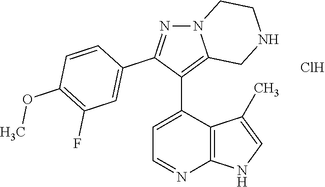 [SMILES: COc1ccc(-c2nn3c(c2-c2ccnc4[nH]cc(C)c24)CNCC3)cc1F ; Cl] | Prepared from tert-butyl 2-(3-fluoro-4- methoxyphenyl)-3-(3-methyl-1H- pyrrolo[2,3-b]pyridin-4-yl)-6,7- dihydropyrazolo[1,5-a]pyrazine-5(4H)- carboxylate (Intermediate 114) LC-MS (Method 2): Rt = 0.88 min; m/z = 378.3 (M + H)+ |
| | 2-(3-fluoro-4-methoxyphenyl)-3-(3- | |
| | methyl-1H-pyrrolo[2,3-b]pyridin-4-yl)- | |
| | 4,5,6,7-tetrahydropyrazolo[1,5- | |
| | a]pyrazine-hydrogen chloride (1/1) | |
| 153 | 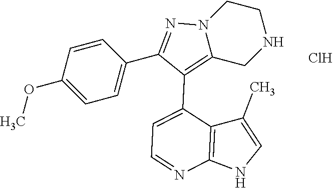 [SMILES: COc1ccc(-c2nn3c(c2-c2ccnc4[nH]cc(C)c24)CNCC3)cc1 ; Cl] | Prepared from tert-butyl 2-(4- methoxyphenyl)-3-(3-methyl-1H- pyrrolo[2,3-b]pyridin-4-yl)-6,7- dihydropyrazolo[1,5-a]pyrazine-5(4H)- carboxylate (Intermediate 115) LC-MS (Method 2): Rt = 0.85 min; m/z = 360.3 (M + H)+ |
| | 2-(4-methoxyphenyl)-3-(3-methyl-1H- | |
| | pyrrolo[2,3-b]pyridin-4-yl)-4,5,6,7- | |
| | tetrahydropyrazolo[1,5-a]pyrazine- | |
| | hydrogen chloride (1/1) | |
| 154 | 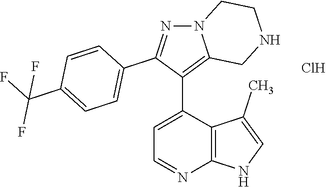 [SMILES: Cc1c[nH]c2nccc(-c3c(-c4ccc(C(F)(F)F)cc4)nn4c3CNCC4)c12 ; Cl] | Produced from tert-butyl 3-(3-methyl-1H- pyrrolo[2,3-b]pyridin-4-yl)-2-[4- (trifluoromethyl)phenyl]-6,7- dihydropyrazolo[1,5-a]pyrazine-5(4H)- carboxylate (Intermediate 116) LC-MS (Method 2): Rt = 1.02 min; m/z = 398.4 (M + H)+ |
| | 3-(3-methyl-1H-pyrrolo[2,3-b]pyridin- | |
| | 4-yl)-2-[4-(trifluoromethyl)phenyl]- | |
| | 4,5,6,7-tetrahydropyrazolo[1,5- | |
| | a]pyrazine-hydrogen chloride (1/1) | |
| 155 | 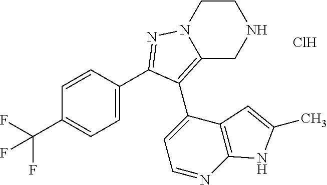 [SMILES: Cc1cc2c(-c3c(-c4ccc(C(F)(F)F)cc4)nn4c3CNCC4)ccnc2[nH]1 ; Cl] | Produced from tert-butyl 3-(2-methyl-1H- pyrrolo[2,3-b]pyridin-4-yl)-2-[4- (trifluoromethyl)phenyl]-6,7- dihydropyrazolo[1,5-a]pyrazine-5(4H)- carboxylate (Intermediate 99) LC-MS (Method 2): Rt = 1.05 min; m/z = 398.3 (M + H)+ |
| | 3-(2-methyl-1H-pyrrolo[2,3-b]pyridin- | |
| | 4-yl)-2-[4-(trifluoromethyl)phenyl]- | |
| | 4,5,6,7-tetrahydropyrazolo[1,5- | |
| | a]pyrazine-hydrogen chloride (1/1) | |
| 156 | 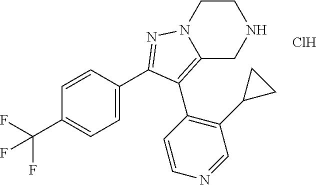 [SMILES: FC(F)(F)c1ccc(-c2nn3c(c2-c2ccncc2C2CC2)CNCC3)cc1 ; Cl] | Produced from tert-butyl 3-(3- cyclopropylpyridin-4-yl)-2-[4- (trifluoromethyl)phenyl]-6,7- dihydropyrazolo[1,5-a]pyrazine-5(4H)- carboxylate (Intermediate 100) LC-MS (Method 2): Rt = 1.09 min; m/z = 385.3 (M + H)+ |
| | 3-(3-cyclopropylpyridin-4-yl)-2-[4- | |
| | (trifluoromethyl)phenyl]-4,5,6,7- | |
| | tetrahydropyrazolo[1,5-a]pyrazine- | |
| | hydrogen chloride (1/1) | |
| 157 | 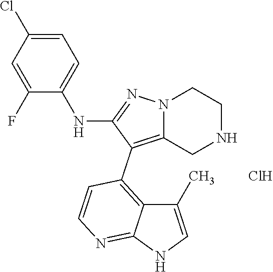 [SMILES: Cc1c[nH]c2nccc(-c3c(Nc4ccc(Cl)cc4F)nn4c3CNCC4)c12 ; Cl] | Produced from tert-butyl 2-(4-chloro-2- fluoroanilino)-3-(3-methyl-1H-pyrrolo[2,3- b]pyridin-4-yl)-6,7-dihydropyrazolo[1,5- a]pyrazine-5(4H)-carboxylate (Intermediate 103) LC-MS (Method 2): Rt = 1.08 min; m/z = 397.2 (M + H)+ |
| | N-(4-chloro-2-fluorophenyl)-3-(3- | |
| | methyl-1H-pyrrolo[2,3-b]pyridin-4-yl)- | |
| | 4,5,6,7-tetrahydropyrazolo[1,5- | |
| | alpyrazin-2-amine-hydrogen chloride | |
| | (1/1) | |
| 158 | 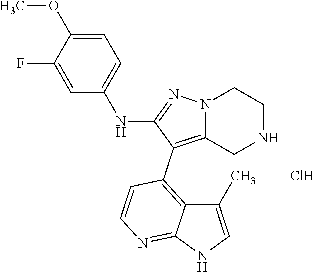 [SMILES: COc1ccc(Nc2nn3c(c2-c2ccnc4[nH]cc(C)c24)CNCC3)cc1F ; Cl] | Produced from tert-butyl 2-(3-fluoro-4- methoxyanilino)-3-(3-methyl-1H- pyrrolo[2,3-b]pyridin-4-yl)-6,7- dihydropyrazolo[1,5-a]pyrazine-5(4H)- carboxylate (Intermediate 106) LC-MS (Method 2): Rt = 0.92 min; m/z = 393.3 (M + H)+ |
| | N-(3-fluoro-4-methoxyphenyl)-3-(3- | |
| | methyl-1H-pyrrolo[2,3-b]pyridin-4-yl)- | |
| | 4,5,6,7-tetrahydropyrazolo[1,5- | |
| | a]pyrazin-2-amine-hydrogen chloride | |
| | (1/1) | |
| 159 | 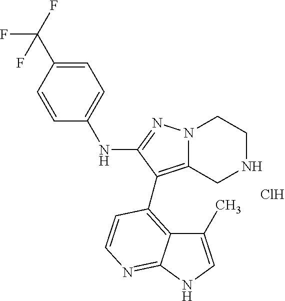 [SMILES: Cc1c[nH]c2nccc(-c3c(Nc4ccc(C(F)(F)F)cc4)nn4c3CNCC4)c12 ; Cl] | Produced from tert-butyl 3-(3-methyl-1H- pyrrolo[2,3-b]pyridin-4-yl)-2-[4- (trifluoromethyl)anilino]-6,7- dihydropyrazolo[1,5-a]pyrazine-5(4H)- carboxylate (Intermediate 109) LC-MS (Method 2): Rt = 1.06 min; m/z = 414.3 (M + H)+ |
| | 3-(3-methyl-1H-pyrrolo[2,3-b]pyridin- | |
| | 4-yl)-N-[4-(trifluoromethyl)phenyl]- | |
| | 4,5,6,7-tetrahydropyrazolo[1,5- | |
| | alpyrazin-2-amine-hydrogen chloride | |
| | (1/1) | |
| 160 | 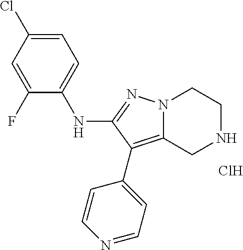 [SMILES: Fc1cc(Cl)ccc1Nc1nn2c(c1-c1ccncc1)CNCC2 ; Cl] | Produced from tert-butyl 2-(4-chloro-2- fluoroanilino)-3-(pyridin-4-yl)-6,7- dihydropyrazolo[1,5-a]pyrazine-5(4H)- carboxylate (Intermediate 110) LC-MS (Method 2): Rt = 0.97 min; m/z = 344.2 (M + H)+ |
| | N-(4-chloro-2-fluorophenyl)-3-(pyridin- | |
| | 4-yl)-4,5,6,7-tetrahydropyrazolo[1,5- | |
| | a]pyrazin-2-amine-hydrogen chloride | |
| | (1/1) | |
| 161 | 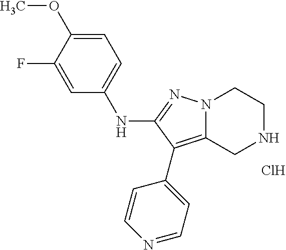 [SMILES: COc1ccc(Nc2nn3c(c2-c2ccncc2)CNCC3)cc1F ; Cl] | Produced from tert-butyl 2-(3-fluoro-4- methoxyanilino)-3-(pyridin-4-yl)-6,7- dihydropyrazolo[1,5-a]pyrazine-5(4H)- carboxylate (Intermediate 111) LC-MS (Method 2): Rt = 0.82 min; m/z = 340.3 (M + H)+ |
| | N-(3-fluoro-4-methoxyphenyl)-3- | |
| | (pyridin-4-yl)-4,5,6,7- | |
| | tetrahydropyrazolo[1,5-a]pyrazin-2- | |
| | amine-hydrogen chloride (1/1) | |
| 162 | 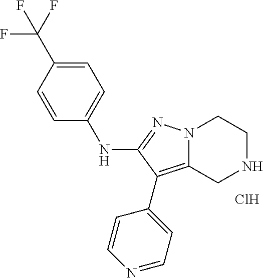 [SMILES: FC(F)(F)c1ccc(Nc2nn3c(c2-c2ccncc2)CNCC3)cc1 ; Cl] | Produced from tert-butyl 3-(pyridin-4-yl)- 2-[4-(trifluoromethyl)anilino]-6,7- dihydropyrazolo[1,5-a]pyrazine-5(4H)- carboxylate (Intermediate 112) LC-MS (Method 2): Rt = 0.99 min; m/z = 360.3 (M + H)+ |
| | 3-(pyridin-4-yl)-N-[4- | |
| | (trifluoromethyl)phenyl]-4,5,6,7- | |
| | tetrahydropyrazolo[1,5-a]pyrazin-2- | |
| | amine-hydrogen chloride (1/1) | |
| 163 | 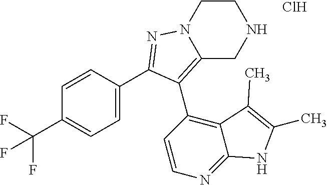 [SMILES: Cc1[nH]c2nccc(-c3c(-c4ccc(C(F)(F)F)cc4)nn4c3CNCC4)c2c1C ; Cl] | Produced from tert-butyl 3-(2,3-dimethyl- 1H-pyrrolo[2,3-b]pyridin-4-yl)-2-[4- (trifluoromethyl)phenyl]-6,7- dihydropyrazolo[1,5-a]pyrazine-5(4H)- carboxylate (Intermediate 122) LC-MS (Method 3): Rt = 1.08 min; m/z = 412.3 (M + H)+ |
| | 3-(2,3-dimethyl-1H-pyrrolo[2,3- | |
| | b]pyridin-4-yl)-2-[4- | |
| | (trifluoromethyl)phenyl]-4,5,6,7- | |
| | tetrahydropyrazolo[1,5-a]pyrazine- | |
| | hydrogen chloride (1/1) | |

The following intermediates were prepared analogous to the preparation of Intermediate 151 starting from the corresponding Boc-protected intermediates by reacting with hydrochloric acid.

| | | LC-MS method |
|---|---|---|
| | Structure | Retention time |
| Intermediate | IUPAC-Name | Mass found |
| 164 | 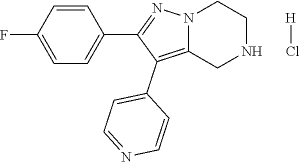 | Produced from tert-butyl 2-(4-fluorophenyl)-3- (pyridin-4-yl)-6,7- dihydropyrazolo[1,5- a]pyrazine-5(4H)- carboxylate (Intermediate 2) Method 3 Rₜ = 0.50 min m/z = 295 [M + H]⁺ |
| | 2-(4-fluorophenyl)-3-(pyridin-4-yl)-4,5,6,7- | |
| | tetrahydropyrazolo[1,5-a]pyrazine-hydrogen | |
| | chloride (1) | |
| 165 | 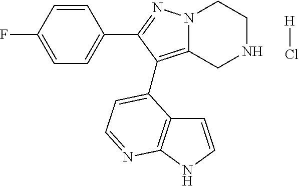 | Produced from tert-butyl 2-(4-fluorophenyl)-3-(1H- pyrrolo[2,3-b]pyridin-4-yl)- 6,7-dihydropyrazolo[1,5- a]pyrazine-5(4H)- carboxylate (Intermediate 35) Method 3. Rₜ = 0.63 min m/z = 334 [M + H]⁺ |
| | 2-(4-fluorophenyl)-3-(1H-pyrrolo[2,3-b]pyridin-4- | |
| | yl)-4,5,6,7-tetrahydropyrazolo[1,5- | |
| | a]pyrazine-hydrogen chloride (1) | |
| 166 | 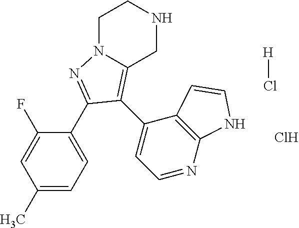 | Produced from tert-butyl 2-(2-fluoro-4- methylphenyl)-3-(1H- pyrrolo[2,3-b]pyridin-4-yl)- 6,7-dihydropyrazolo[1,5- a]pyrazine-5(4H)- carboxylate (Intermediate 123) Method 2 Rₜ = 0.80 min m/z = 348 [M + H]⁺ |
| | 2-(2-fluoro-4-methylphenyl)-3-(1H-pyrrolo[2,3- | |
| | b]pyridin-4-yl)-4,5,6,7-tetrahydropyrazolo[1,5- | |
| | a]pyrazine-hydrogen chloride (1/2) | |
| 167 | 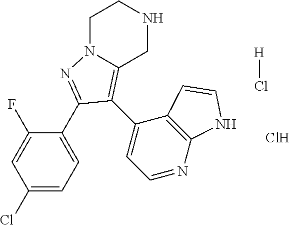 | Produced from tert-butyl 2-(4-chloro-2- fluorophenyl)-3-(1H- pyrrolo[2,3-b]pyridin-4-yl)- 6,7-dihydropyrazolo[1,5- a]pyrazine-5(4H)- carboxylate (Intermediate 124) Method 2 Rₜ = 0.85 min m/z = 368 [M + H]⁺ |
| | 2-(4-chloro-2-fluorophenyl)-3-(1H-pyrrolo[2,3- | |
| | b]pyridin-4-yl)-4,5,6,7-tetrahydropyrazolo[1,5- | |
| | a]pyrazine-hydrogen chloride (1/2) | |
| 168 | 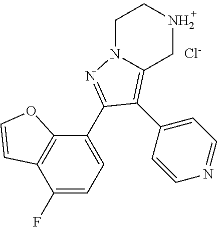 [SMILES: Fc1ccc(-c2nn3c(c2-c2ccncc2)CNCC3)c2occc12 ; [Cl-]] | Produced from tert-butyl 2-(4-fluoro-1-benzofuran- 7-yl)-3-(pyridin-4-yl)-6,7- dihydropyrazolo[1,5- a]pyrazine-5(4H)- carboxylate (Intermediate 125) Method 2 Rₜ = 0.89 min m/z = 335 [M + H]⁺ |
| | 2-(4-fluoro-1-benzofuran-7-yl)-3-(pyridin-4-yl)- | |
| | 4,5,6,7-tetrahydropyrazolo[1,5-a]pyrazin-5-ium | |
| | chloride | |
| 169 | 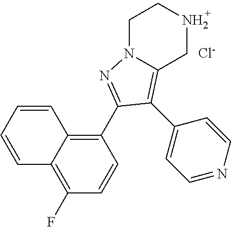 [SMILES: Fc1ccc(-c2nn3c(c2-c2ccncc2)CNCC3)c2ccccc12 ; [Cl-]] | Produced from tert-butyl 2-(4-fluoro-1-naphthyl)-3- (pyridin-4-yl)-6,7- dihydropyrazolo[1,5- a]pyrazine-5(4H)- carboxylate (Intermediate 126) Method 2 Rₜ = 0.95 min m/z = 346 [M + H]⁺ |
| | 2-(4-fluoronaphthalen-1-yl)-3-(pyridin-4-yl)- | |
| | 4,5,6,7-tetrahydropyrazolo[1,5-a]pyrazin-5-ium | |
| | chloride | |
| 170 | 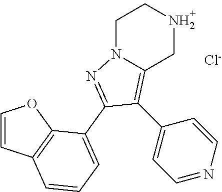 [SMILES: c1cc(-c2nn3c(c2-c2ccncc2)CNCC3)c2occc2c1 ; [Cl-]] | Produced from tert-butyl 2-(1-benzofuran-7-yl)-3- (pyridin-4-yl)-6,7- dihydropyrazolo[1,5- a]pyrazine-5(4H)- carboxylate (Intermediate 127) Method 2 Rₜ = 0.82 min m/z = 318 [M + H]⁺ |
| | 2-(1-benzofuran-7-yl)-3-(pyridin-4-yl)-4,5,6,7- | |
| | tetrahydropyrazolo[1,5-a]pyrazin-5-ium chloride | |
| 171 | 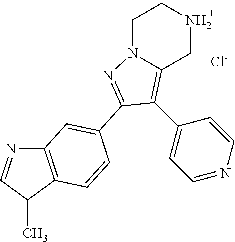 [SMILES: CC1C=Nc2cc(-c3nn4c(c3-c3ccncc3)CNCC4)ccc21 ; [Cl-]] | Produced from tert-butyl 2-(3-methyl-3H-indol-6- yl)-3-(pyridin-4-yl)-6,7- dihydropyrazolo[1,5- a]pyrazine-5(4H)- carboxylate (Intermediate 128) Method 2 Rₜ = 0.87 min m/z = 331 [M + H]⁺ |
| | 2-(3-methyl-3H-indol-6-yl)-3-(pyridin-4-yl)-4,5,6,7- | |
| | tetrahydropyrazolo[1,5-a]pyrazin-5-ium chloride | |
| 172 | 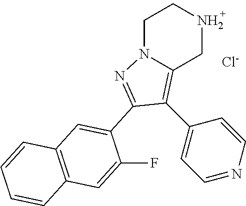 [SMILES: Fc1cc2ccccc2cc1-c1nn2c(c1-c1ccncc1)CNCC2 ; [Cl-]] | Produced from tert-butyl 2-(3-fluoro-2-naphthyl)-3- (pyridin-4-yl)-6,7- dihydropyrazolo[1,5- ajpyrazine-5(4H)- carboxylate (Intermediate 129) Method 2 Rₜ = 0.96 min m/z = 346 [M + H]⁺ |
| | 2-(3-fluoronaphthalen-2-yl)-3-(pyridin-4-yl)- | |
| | 4,5,6,7-tetrahydropyrazolo[1,5-a]pyrazin-5-ium | |
| | chloride | |
| 173 | 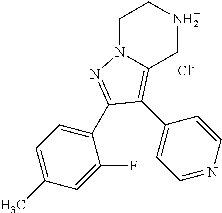 [SMILES: Cc1ccc(-c2nn3c(c2-c2ccncc2)CNCC3)c(F)c1 ; [Cl-]] | Produced from tert-butyl 2-(2-fluoro-4- methylphenyl)-3-(pyridin- 4-yl)-6,7- dihydropyrazolo[1,5- a]pyrazine-5(4H)- carboxylate (Intermediate 130) Method 2 Rₜ = 0.95 min m/z = 310 [M + H]⁺ |
| | 2-(2-fluoro-4-methylphenyl)-3-(pyridin-4-yl)- | |
| | 4,5,6,7-tetrahydropyrazolo[1,5-a]pyrazin-5-ium | |
| | chloride | |
| 174 | 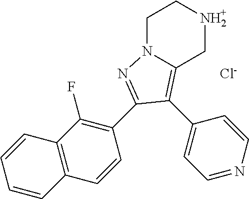 [SMILES: Fc1c(-c2nn3c(c2-c2ccncc2)CNCC3)ccc2ccccc12 ; [Cl-]] | Produced from tert-butyl 2-(1-fluoro-2-naphthyl)-3- (pyridin-4-yl)-6,7- dihydropyrazolo[1,5- a]pyrazine-5(4H)- carboxylate (Intermediate 131) Method 2 Rₜ = 0.97 min m/z = 346 [M + H]⁺ |
| | 2-(1-fluoronaphthalen-2-yl)-3-(pyridin-4-yl)- | |
| | 4,5,6,7-tetrahydropyrazolo[1,5-a]pyrazin-5-ium | |
| | chloride | |
| 175 | 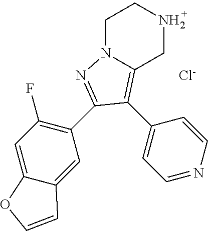 [SMILES: Fc1cc2occc2cc1-c1nn2c(c1-c1ccncc1)CNCC2 ; [Cl-]] | Produced from tert-butyl 2-(6-fluoro-1-benzofuran- 5-yl)-3-(pyridin-4-yl)-6,7- dihydropyrazolo[1,5- a]pyrazine-5(4H)- carboxylate (Intermediate 132) Method 2 Rₜ = 0.86 min m/z = 336 [M + H]⁺ |
| | 2-(6-fluoro-1-benzofuran-5-yl)-3-(pyridin-4-yl)- | |
| | 4,5,6,7-tetrahydropyrazolo[1,5-a]pyrazin-5-ium | |
| | chloride | |
| 176 | 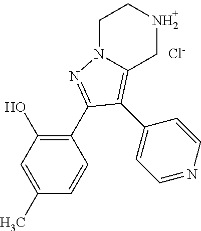 [SMILES: Cc1ccc(-c2nn3c(c2-c2ccncc2)CNCC3)c(O)c1 ; [Cl-]] | Produced from tert-butyl 2-(2-hydroxy-4- methylphenyl)-3-(pyridin- 4-yl)-6,7- dihydropyrazolo[1,5- a]pyrazine-5(4H)- carboxylate (Intermediate 133) Method 2 Rₜ = 0.86 min m/z = 308 [M + H]⁺ |
| | 2-(2-hydroxy-4-methylphenyl)-3-(pyridin-4-yl)- | |
| | 4,5,6,7-tetrahydropyrazolo[1,5-a]pyrazin-5-ium | |
| | chloride | |
| 177 | 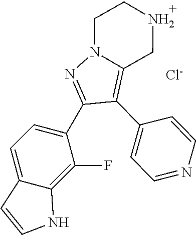 [SMILES: Fc1c(-c2nn3c(c2-c2ccncc2)CNCC3)ccc2cc[nH]c12 ; [Cl-]] | Produced from tert-butyl 2-(7-fluoro-1H-indol-6-yl)- 3-(pyridin-4-yl)-6,7- dihydropyrazolo[1,5- a]pyrazine-5(4H)- carboxylate (Intermediate 134) Method 2 Rₜ = 0.81 min m/z = 335 [M + H]⁺ |
| | 2-(7-fluoro-1H-indol-6-yl)-3-(pyridin-4-yl)-4,5,6,7- | |
| | tetrahydropyrazolo[1,5-a]pyrazin-5-ium chloride | |
| 178 | 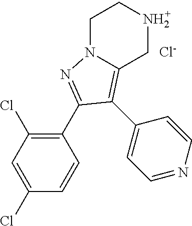 [SMILES: Clc1ccc(-c2nn3c(c2-c2ccncc2)CNCC3)c(Cl)c1 ; [Cl-]] | Produced from tert-butyl 2-(2,4-dichlorophenyl)-3- (pyridin-4-yl)-6,7- dihydropyrazolo[1,5- a]pyrazine-5(4H)- carboxylate (Intermediate 135) Method 2 Rₜ = 0.96 min m/z = 346 [M + H]⁺ |
| | 2-(2,4-dichlorophenyl)-3-(pyridin-4-yl)-4,5,6,7- | |
| | tetrahydropyrazolo[1,5-a]pyrazin-5-ium chloride | |
| 179 | 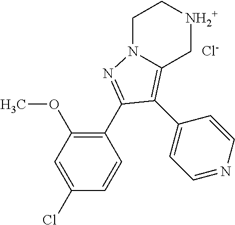 [SMILES: COc1cc(Cl)ccc1-c1nn2c(c1-c1ccncc1)C[NH2+]CC2 ; [Cl-]] | Produced from tert-butyl 2-(4-chloro-2- methoxyphenyl)-3- (pyridin-4-yl)-6,7- dihydropyrazolo[1,5- a]pyrazine-5(4H)- carboxylate (Intermediate 136) Method 2 Rₜ = 0.89 min m/z = 342 [M + H]⁺ |
| | 2-(4-chloro-2-methoxyphenyl)-3-(pyridin-4-yl)- | |
| | 4,5,6,7-tetrahydropyrazolo[1,5-a]pyrazin-5-ium | |
| | chloride | |
| 180 | 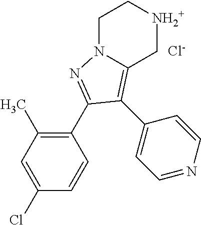 [SMILES: Cc1cc(Cl)ccc1-c1nn2c(c1-c1ccncc1)C[NH2+]CC2 ; [Cl-]] | Produced from tert-butyl 2-(4-chloro-2- methylphenyl)-3-(pyridin- 4-yl)-6,7- dihydropyrazolo[1,5- a]pyrazine-5(4H)- carboxylate (Intermediate 137) Method 2 Rₜ = 0.96 min m/z = 326 [M + H]⁺ |
| | 2-(4-chloro-2-methylphenyl)-3-(pyridin-4-yl)- | |
| | 4,5,6,7-tetrahydropyrazolo[1,5-a]pyrazin-5-ium | |
| | chloride | |
| 181 | 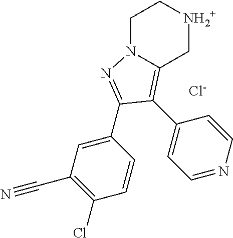 [SMILES: N#Cc1cc(-c2nn3c(c2-c2ccncc2)C[NH2+]CC3)ccc1Cl ; [Cl-]] | Produced from tert-butyl 2-(4-chloro-3- cyanophenyl)-3-(pyridin- 4-yl)-6,7- dihydropyrazolo[1,5- a]pyrazine-5(4H)- carboxylate (Intermediate 138) Method 2 Rₜ = 0.90 min m/z = 337 [M + H]⁺ |
| | 2-(4-chloro-3-cyanophenyl)-3-(pyridin-4-yl)- | |
| | 4,5,6,7-tetrahydropyrazolo[1,5-a]pyrazin-5-ium | |
| | chloride | |
| 182 | 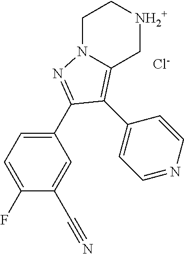 [SMILES: N#Cc1cc(-c2nn3c(c2-c2ccncc2)C[NH2+]CC3)ccc1F ; [Cl-]] | Produced from tert-butyl 2-(3-cyano-4- fluorophenyl)-3-(pyridin- 4-yl)-6,7- dihydropyrazolo[1,5- a]pyrazine-5(4H)- carboxylate (Intermediate 139) Method 2 Rₜ = 0.84 min m/z = 321 [M + H]⁺ |
| | 2-(3-cyano-4-fluorophenyl)-3-(pyridin-4-yl)- | |
| | 4,5,6,7-tetrahydropyrazolo[1,5-a]pyrazin-5-ium | |
| | chloride | |
| 183 | 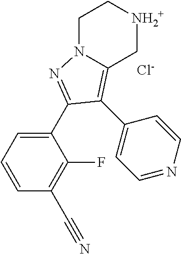 [SMILES: N#Cc1cccc(-c2nn3c(c2-c2ccncc2)C[NH2+]CC3)c1F ; [Cl-]] | Produced from tert-butyl 2-(3-cyano-2- fluorophenyl)-3-(pyridin- 4-yl)-6,7- dihydropyrazolo[1,5- a]pyrazine-5(4H)- carboxylate (Intermediate 140) Method 2 Rₜ = 0.78 min m/z = 321 [M + H]⁺ |
| | 2-(3-cyano-2-fluorophenyl)-3-(pyridin-4-yl)- | |
| | 4,5,6,7-tetrahydropyrazolo[1,5-a]pyrazin-5-ium | |
| | chloride | |
| 184 | 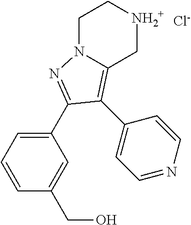 [SMILES: OCc1cccc(-c2nn3c(c2-c2ccncc2)C[NH2+]CC3)c1 ; [Cl-]] | Produced from tert-butyl 2-[3- (hydroxymethyl)phenyl]- 3-(pyridin-4-yl)-6,7- dihydropyrazolo[1,5- a]pyrazine-5(4H)- carboxylate (Intermediate 145) Method 3 Rₜ = 0.46 min m/z = 307 [M + H]⁺ |
| | 2-[3-(hydroxymethyl)phenyl]-3-(pyridin-4-yl)- | |
| | 4,5,6,7-tetrahydropyrazolo[1,5-a]pyrazin-5-ium | |
| | chloride | |
| 185 | 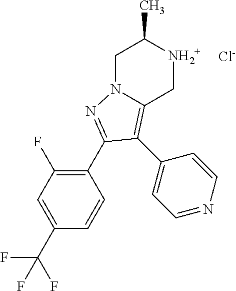 [SMILES: C[C@@H]1Cn2nc(-c3ccc(C(F)(F)F)cc3F)c(-c3ccncc3)c2C[NH2+]1 ; [Cl-]] | Produced from tert-butyl (6R)-2-[2-fluoro-4- (trifluoromethyl)phenyl]-6- methyl-3-(pyridin-4-yl)- 6,7-dihydropyrazolo[1,5- a]pyrazine-5(4H)- carboxylate (Intermediate 142) Method 2 Rₜ = 1.06 min m/z = 378 [M + H]⁺ |
| | (6R)-2-[2-fluoro-4-(trifluoromethyl)phenyl]-6- | |
| | methyl-3-(pyridin-4-yl)-4,5,6,7- | |
| | tetrahydropyrazolo[1,5-a]pyrazin-5-ium chloride | |
| 186 | 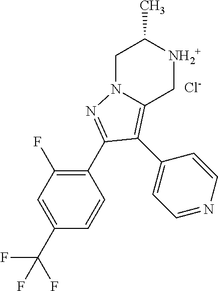 [SMILES: C[C@H]1Cn2nc(-c3ccc(C(F)(F)F)cc3F)c(-c3ccncc3)c2C[NH2+]1 ; [Cl-]] | Produced from tert-butyl (6S)-2-[2-fluoro-4- (trifluoromethyl)phenyl]- 6-methyl-3-(pyridin-4- yl)-6,7- dihydropyrazolo[1,5- a]pyrazine-5(4H)- carboxylate (Intermediate 141) Method 2 Rₜ = 1.06 min m/z = 378 [M + H]⁺ |
| | (6S)-2-[2-fluoro-4-(trifluoromethyl)phenyl]-6- | |
| | methyl-3-(pyridin-4-yl)-4,5,6,7- | |
| | tetrahydropyrazolo[1,5-a]pyrazin-5-ium chloride | |
| 187 | 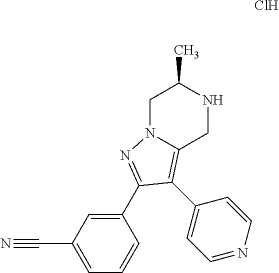 [SMILES: Cl ; C[C@@H]1Cn2nc(-c3cccc(C#N)c3)c(-c3ccncc3)c2CN1] | Produced from tert- butyl (6R)-2-(3- cyanophenyl)-6- methyl-3-(pyridin-4- yl)-6,7- dihydropyrazolo[1,5- a]pyrazine-5(4H)- carboxylate (Intermediate 143) Method 2 Rₜ = 0.85 min m/z = 316 [M + H]⁺ |
| | 3-[(6R)-6-methyl-3-(pyridin-4-yl)-4,5,6,7- | |
| | tetrahydropyrazolo[1,5-a]pyrazin-2-yl]benzonitrile- | |
| | hydrogen chloride (1/1) | |
| 188 | 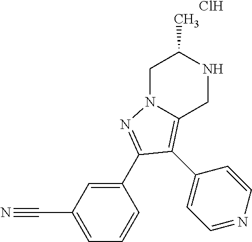 [SMILES: Cl ; C[C@H]1Cn2nc(-c3cccc(C#N)c3)c(-c3ccncc3)c2CN1] | Produced from tert- butyl (6S)-2-(3- cyanophenyl)-6- methyl-3-(pyridin-4- yl)-6,7- dihydropyrazolo[1,5- a]pyrazine-5(4H)- carboxylate (Intermediate 144) Method 2 Rₜ = 0.85 min m/z = 316 [M + H]⁺ |
| | 3-[(6S)-6-methyl-3-(pyridin-4-yl)-4,5,6,7- | |
| | tetrahydropyrazolo[1,5-a]pyrazin-2-yl]benzonitrile- | |
| | hydrogen chloride (1/1) | |

The following intermediates were prepared analogously to the synthesis of Intermediate 45.

| | Structure |
|---|---|
| | IUPAC-Name |
| | LC-MS (method): Retention time; Mass found |
| Example | ¹H-NMR |
| @ExampleNo | 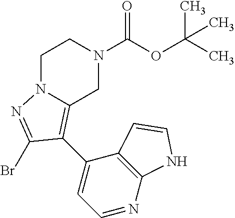 [SMILES: CC(C)(C)OC(=O)N1CCn2nc(Br)c(-c3ccnc4[nH]ccc34)c2C1] |
| | tert-butyl 2-bromo-3-(1H-pyrrolo[2,3-b]pyridin-4-yl)-6,7- |
| | dihydropyrazolo[1,5-a]pyrazine-5(4H)-carboxylate |
| | Produced from tert-butyl 2-bromo-3-iodo-6,7-dihydropyrazolo[1,5- |
| | alpyrazine-5(4H)-carboxylate (Intermediate 67) |
| | LC-MS (Method 3): Rₜ = 1.04 min; MS (ESIpos): m/z = 419 [M + H]⁺ |

Intermediate 189

### (2E)-4-bromo-1-[2-(4-fluorophenyl)-3-(pyridin-4-yl)-6,7-dihydropyrazolo[1,5-a]pyrazin-5(4H)-yl]but-2-en-1-one

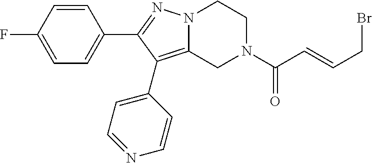 [SMILES: O=C(/C=C/CBr)N1CCn2nc(-c3ccc(F)cc3)c(-c3ccncc3)c2C1]

To a solution of 2-(4-fluorophenyl)-3-(pyridin-4-yl)-4,5,6,7-tetrahydropyrazolo[1,5-a]pyrazine (170 mg, 578 μmol, Intermediate 3) in dry DMF (8.2 ml) was added (2E)-4-bromobut-2-enoic acid (114 mg, 693 μmol; CAS-RN: [13991-36-1]), then N,N-diisopropylethylamine (600 μl, 3.5 mmol) followed by 2,4,6-tripropyl-1,3,5,2 lambda5,4lambda5,6lambda5-trioxatriphosphinane-2,4,6-trione (solution in DMF, 510 μl, 50% purity, 870 μmol) and the mixture was stirred at rt for 15 min. The reaction mixture was used for next reactions without further purification.

LC-MS (Method 2): Rₜ=1.07 min; MS (ESIpos): m/z=443.2 [M+H]⁺

### Intermediate 190

### tert-butyl 2-[4-(difluoromethoxy)phenyl]-3-(pyridin-4-yl)-6,7-dihydropyrazolo[1,5-a]pyrazine-5(4H)-carboxylate

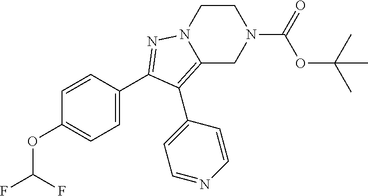 [SMILES: CC(C)(C)OC(=O)N1CCn2nc(-c3ccc(OC(F)F)cc3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and [4-(difluoromethoxy)phenyl]boronic acid (297 mg, 1.58 mmol) in 1,4-dioxane (8.0 ml) and water (2.0 ml) were added sodium carbonate (223 mg, 2.10 mmol) and (1,1′-bis(diphenylphosphino) ferrocene) palladium (II) dichloride (77.1 mg, 0.105 mmol) at 20° C. The mixture was degassed with nitrogen and stirred at 90° C. for 16 hours. The reaction mixture was poured into water and extracted with ethyl acetate. The combined organic layers were washed with brine, dried over anhydrous sodium sulfate, filtered and the filtrate was concentrated in vacuo to give tert-butyl 2-[4-(difluoromethoxy)phenyl]-3-(pyridin-4-yl)-6,7-dihydropyrazolo[1,5-a]pyrazine-5(4H)-carboxylate (400 mg, 0.904 mmol, 85% yield) as a yellow oil.

LC-MS (Method 20): Rₜ=0.671 min; MS (ESIpos): m/z=442.1 [M+H]⁺

### Intermediate 191

### 2-(4-(difluoromethoxy)phenyl)-3-(pyridin-4-yl)-4,5,6,7-tetrahydropyrazolo[1,5-a]pyrazine hydrochloride (1:1)

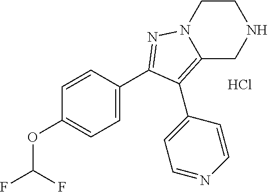 [SMILES: FC(F)Oc1ccc(-c2nn3c(c2-c2ccncc2)CNCC3)cc1 ; Cl]

To a solution of tert-butyl 2-[4-(difluoromethoxy)phenyl]-3-(pyridin-4-yl)-6,7-dihydropyrazolo[1,5-a]pyrazine-5(4H)-carboxylate (400 mg, 0.904 mmol, Intermediate 190) in ethyl acetate (8 ml) was added hydrochloric acid (10 ml, 4M in ethyl acetate) at 20° C. The mixture was stirred at 20° C. for 1 hour. The reaction mixture was filtered and washed with ethyl acetate, the solid cake was collected to give 2-(4-(difluoromethoxy)phenyl)-3-(pyridin-4-yl)-4,5,6,7-tetrahydropyrazolo[1,5-a]pyrazine hydrochloride (1:1) (300 mg, 0.791 mmol, 87% yield) as a brown solid.

LC-MS (Method 16): Rₜ=0.391 min; MS (ESIpos): m/z=342.1 [M+H]⁺.

### Intermediate 192

### tert-butyl 3-(pyridin-4-yl)-2-[4-(trifluoromethoxy)phenyl]-6,7-dihydropyrazolo[1,5-a]pyrazine-5(4H)-carboxylate

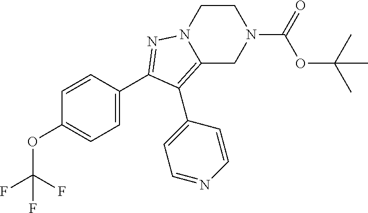 [SMILES: CC(C)(C)OC(=O)N1CCn2nc(-c3ccc(OC(F)(F)F)cc3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and [4-(trifluoromethoxy)phenyl]boronic acid (325 mg, 1.58 mmol) in 1,4-dioxane (8.0 ml) and water (2.0 ml) were added (1,1′-bis(diphenylphosphino) ferrocene) palladium (II) dichloride (77.1 mg, 0.105 mmol) and sodium carbonate (223 mg, 2.10 mmol) at 20° C. The reaction mixture was degassed with nitrogen and stirred at 90° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl acetate=1:0 to 3:1) to give tert-butyl 3-(pyridin-4-yl)-2-[4-(trifluoromethoxy)phenyl]-6,7-dihydropyrazolo[1,5-a]pyrazine-5(4H)-carboxylate (360 mg, 0.781 mmol, 74% yield) as a yellow oil.

LC-MS (Method 16): Rₜ=0.555 min; MS (ESIpos): m/z=460.1 [M+H]⁺.

### Intermediate 193

### 3-(pyridin-4-yl)-2-[4-(trifluoromethoxy)phenyl]-4,5,6,7-tetrahydropyrazolo[1,5-a]pyrazine hydrochloride (1:1)

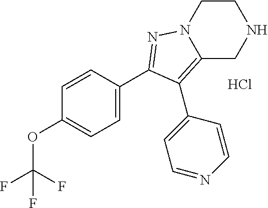 [SMILES: FC(F)(F)Oc1ccc(-c2nn3c(c2-c2ccncc2)CNCC3)cc1 ; Cl]

To a solution of tert-butyl 3-(pyridin-4-yl)-2-[4-(trifluoromethoxy)phenyl]-6,7-dihydropyrazolo[1,5-a]pyrazine-5(4H)-carboxylate (360 mg, 0.781 mmol, Intermediate 192) in ethyl acetate (5.0 ml) was added hydrochloric acid (3.0 ml, 4M in ethyl acetate) at 20° C. The mixture was stirred at 20° C. for 1 hour. The reaction mixture was filtered and the solid cake was collected to give 3-(pyridin-4-yl)-2-[4-(trifluoromethoxy)phenyl]-4,5,6,7-tetrahydropyrazolo[1,5-a]pyrazine hydrochloride (1:1) (220 mg, 0.554 mmol, 70% yield) as a brown solid.

LC-MS (Method 16): Rₜ=0.435 min; MS (ESIpos): m/z=360.1 [M+H]⁺.

### Intermediate 194

### tert-butyl 2-(4-ethoxyphenyl)-3-(pyridin-4-yl)-6,7-dihydropyrazolo[1,5-a]pyrazine-5(4H)-carboxylate

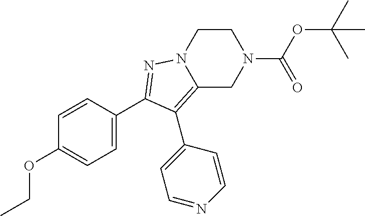 [SMILES: CCOc1ccc(-c2nn3c(c2-c2ccncc2)CN(C(=O)OC(C)(C)C)CC3)cc1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and (4-ethoxyphenyl) boronic acid (262 mg, 1.58 mmol) in 1,4-dioxane (8.0 ml) and water (2.0 ml) were added sodium carbonate (223 mg, 2.10 mmol) and (1,1′-bis(diphenylphosphino) ferrocene) palladium (II) dichloride (77.1 mg, 0.105 mmol) at 20° C. The mixture was degassed with nitrogen and stirred at 90° C. for 16 hours. The reaction solution was poured into water and extracted with ethyl acetate. The combined organic layers were washed with brine, dried over anhydrous sodium sulfate, filtered and the filtrate was concentrated in vacuo to give tert-butyl 2-(4-ethoxyphenyl)-3-(pyridin-4-yl)-6,7-dihydropyrazolo[1,5-a]pyrazine-5(4H)-carboxylate (400 mg, 0.951 mmol, 90% yield) as a yellow oil.

LC-MS (Method 20): Rₜ=0.675 min; MS (ESIpos): m/z=420.2 [M+H]⁺.

### Intermediate 195

### 2-(4-ethoxyphenyl)-3-(pyridin-4-yl)-4,5,6,7-tetrahydropyrazolo[1,5-a]pyrazine hydrochloride (1:1)

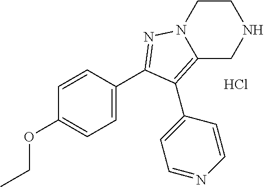 [SMILES: CCOc1ccc(-c2nn3c(c2-c2ccncc2)CNCC3)cc1 ; Cl]

To a solution of tert-butyl 2-(4-ethoxyphenyl)-3-(pyridin-4-yl)-6,7-dihydropyrazolo[1,5-a]pyrazine-5(4H)-carboxylate (400 mg, 0.951 mmol, Intermediate 194) in ethyl acetate (8.0 ml) was added hydrochloric acid (10.0 ml, 4M in ethyl acetate) at 20° C. The mixture was stirred at 20° C. for 1 hour. The reaction mixture was filtered and washed with ethyl acetate. The solid cake was collected to give 2-(4-ethoxyphenyl)-3-(pyridin-4-yl)-4,5,6,7-tetrahydropyrazolo[1,5-a]pyrazine hydrochloride (1:1) (300 mg, 0.840 mmol, 88% yield) as a brown solid.

LC-MS (Method 16): Rₜ=0.385 min; MS (ESIpos): m/z=320.1 [M+H]⁺.

### Intermediate 196

### tert-butyl 2-[4-(difluoromethyl)phenyl]-3-(pyridin-4-yl)-6,7-dihydropyrazolo[1,5-a]pyrazine-5(4H)-carboxylate

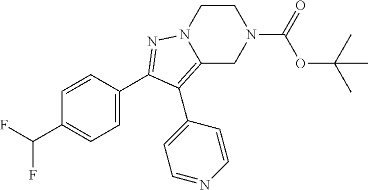 [SMILES: CC(C)(C)OC(=O)N1CCn2nc(-c3ccc(C(F)F)cc3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 1.05 mmol, Intermediate 68) and mg, [4-(difluoromethyl)phenyl]boronic acid (272 mg, 1.58 mmol) in 1,4-dioxane (8 ml) and water (2 ml) were added (1,1′-bis(diphenylphosphino) ferrocene) palladium (II) dichloride (77.2 mg, 0.105 mmol) and sodium carbonate (224 mg, 2.11 mmol) at 20° C. The mixture was degassed with nitrogen and stirred at 90° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl acetate=1:0 to 3:1) to give tert-butyl 2-[4-(difluoromethyl)phenyl]-3-(pyridin-4-yl)-6,7-dihydropyrazolo[1,5-a]pyrazine-5(4H)-carboxylate (310 mg, 0.726 mmol, 68% yield) as a yellow oil.

LC-MS (Method 16): Rₜ=0.694 min; MS (ESIpos): m/z=426.1 [M+H]⁺.

### Intermediate 197

### 2-[4-(difluoromethyl)phenyl]-3-(pyridin-4-yl)-4,5,6,7-tetrahydropyrazolo[1,5-a]pyrazine hydrochloride (1:1)

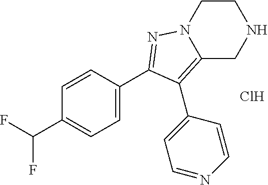 [SMILES: FC(F)c1ccc(-c2nn3c(c2-c2ccncc2)CNCC3)cc1 ; Cl]

To a solution of tert-butyl 2-[4-(difluoromethyl)phenyl]-3-(pyridin-4-yl)-6,7-dihydropyrazolo[1,5-a]pyrazine-5(4H)-carboxylate (310 mg, 0.727 mmol, Intermediate 196) in ethyl acetate (5.0 ml) was added hydrochloric acid (3.0 ml, 4M in ethyl acetate) at 20° C. The mixture was stirred at 20° C. for 1 hour. The reaction mixture was concentrated in vacuo to give 2-[4-(difluoromethyl)phenyl]-3-(pyridin-4-yl)-4,5,6,7-tetrahydropyrazolo[1,5-a]pyrazine hydrochloride (1:1) (220 mg, 0.606 mmol, 83% yield) as a yellow oil.

LC-MS (Method 16): Rₜ=0.395 min; MS (ESIpos): m/z=326.1 [M+H]⁺,

### Intermediate 198

### tert-butyl 2-(5-chloro-2-fluorophenyl)-3-(pyridin-4-yl)-6,7-dihydropyrazolo[1,5-a]pyrazine-5(4H)-carboxylate

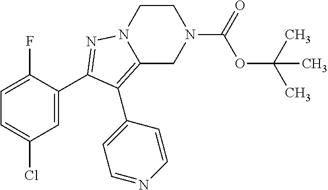 [SMILES: CC(C)(C)OC(=O)N1CCn2nc(-c3cc(Cl)ccc3F)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and (5-chloro-2-fluorophenyl) boronic acid (276 mg, 1.58 mmol) in 1,4-dioxane (8.0 ml) and water (2.0 ml) were added (1,1′-bis(diphenylphosphino) ferrocene) palladium (II) dichloride (77.2 mg, 0.105 mmol) and sodium carbonate (224 mg, 2.11 mmol) at 20° C. The mixture was degassed with nitrogen and stirred at 90° C. for 16 hours. The reaction solution was poured into water and extracted with ethyl acetate. The combined organic layers were washed with birne, dried over anhydrous sodium sulfate, filtered and the filtrate was concentrated under vacuo to give a residue. The residue was purified by silica gel column chromatography (petroleum ether:ethyl acetate=1:0 to 3:1) to give tert-butyl 2-(5-chloro-2-fluorophenyl)-3-(pyridin-4-yl)-6,7-dihydropyrazolo[1,5-a]pyrazine-5(4H)-carboxylate (400 mg, 0.932 mmol, 88% yield) as a yellow oil.

LC-MS (Method 16): Rₜ=0.497 min; MS (ESIpos): m/z=428.1 [M+H]⁺.

### Intermediate 199

### 2-(5-chloro-2-fluorophenyl)-3-(pyridin-4-yl)-4,5,6,7-tetrahydropyrazolo[1,5-a]pyrazine hydrochloride (1:1)

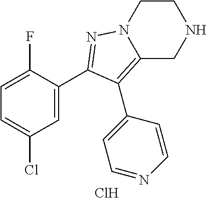 [SMILES: Fc1ccc(Cl)cc1-c1nn2c(c1-c1ccncc1)CNCC2 ; Cl]

To a solution of tert-butyl 2-(5-chloro-2-fluorophenyl)-3-(pyridin-4-yl)-6,7-dihydropyrazolo[1,5-a]pyrazine-5(4H)-carboxylate (400 mg, 0.933 mmol, Intermediate 198) in ethyl acetate (10 ml) was added hydrochloric acid (8.0 ml, 4M in ethyl acetate) at 20° C. The mixture was stirred at 20° C. for 1 hour. The reaction mixture was filtered and the solid cake was collected to give 2-(5-chloro-2-fluorophenyl)-3-(pyridin-4-yl)-4,5,6,7-tetrahydropyrazolo[1,5-a]pyrazine hydrochloride (1:1) (300 mg, 0.821 mmol, 88% yield) as a brown oil.

LC-MS (Method 16): Rₜ=0.399 min; MS (ESIpos): m/z=328.0 [M+H]⁺.

### Intermediate 200

### tert-butyl 2-(3-chlorophenyl)-3-(pyridin-4-yl)-6,7-dihydropyrazolo[1,5-a]pyrazine-5(4H)-carboxylate

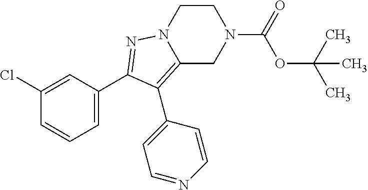 [SMILES: CC(C)(C)OC(=O)N1CCn2nc(-c3cccc(Cl)c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and (3-chlorophenyl) boronic acid (247 mg, 1.58 mmol) in 1,4-dioxane (8.0 ml) and water (2.0 ml) were added (1,1′-bis(diphenylphosphino) ferrocene) palladium (II) dichloride (77.2 mg, 0.105 mmol) and sodium carbonate (224 mg, 2.11 mmol) at 20° C. The mixture was degassed with nitrogen and stirred at 90° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl acetate=1:0 to 3:1) to give tert-butyl 2-(3-chlorophenyl)-3-(pyridin-4-yl)-6,7-dihydropyrazolo[1,5-a]pyrazine-5(4H)-carboxylate (320 mg, 0.778 mmol, 73% yield) as a yellow oil.

LC-MS (Method 16): Rₜ=0.502 min; MS (ESIpos): m/z=410.1 [M+H]⁺.

### Intermediate 201

### 2-(3-chlorophenyl)-3-(pyridin-4-yl)-4,5,6,7-tetrahydropyrazolo[1,5-a]pyrazine hydrochloride (1:1)

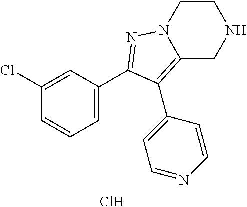 [SMILES: Clc1cccc(-c2nn3c(c2-c2ccncc2)CNCC3)c1 ; Cl]

To a solution of tert-butyl 2-(3-chlorophenyl)-3-(pyridin-4-yl)-6,7-dihydropyrazolo[1,5-a]pyrazine-5(4H)-carboxylate (320 mg, 0.779 mmol, Intermediate 200) in ethyl acetate (5.0 ml) was added hydrochloric acid (3.0 ml, 4M in ethyl acetate) at 20° C. The mixture was stirred at 20° C. for 1 hour. The reaction mixture was concentrated in vacuo to give 2-(3-chlorophenyl)-3-(pyridin-4-yl)-4,5,6,7-tetrahydropyrazolo[1,5-a]pyrazine hydrochloride (1:1) (200 mg, 0.575 mmol, 73% yield) as a brown oil.

LC-MS (Method 16): Rₜ=0.380 min; MS (ESIpos): m/z=310.1 [M+H]⁺.

### Intermediate 202

### tert-butyl 2-[3-fluoro-4-(trifluoromethyl)phenyl]-3-(pyridin-4-yl)-6,7-dihydropyrazolo[1,5-a]pyrazine-5(4H)-carboxylate

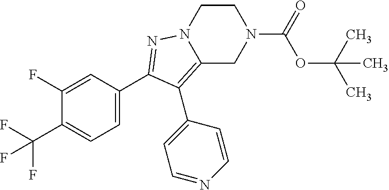 [SMILES: CC(C)(C)OC(=O)N1CCn2nc(-c3ccc(C(F)(F)F)c(F)c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and [3-fluoro-4-(trifluoromethyl)phenyl]boronic acid (329 mg, 1.58 mmol) in 1,4-dioxane (8.0 ml) and water (2.0 ml) were added (1,1′-bis(diphenylphosphino) ferrocene) palladium (II) dichloride (77.2 mg, 0.105 mmol) and sodium carbonate (224 mg, 2.11 mmol) at 20° C. The mixture was degassed with nitrogen and stirred at 90° C. for 16 hours. The reaction mixture was poured into water and washed with ethyl acetate. The combined organic layers were dried over anhydrous sodium sulfate, filtered and the filtrate was concentrated in vacuo to give a residue. The residue was purified by silica gel column chromatography (petroleum ether:ethyl acetate=0:1 to 3:1) to give tert-butyl 2-[3-fluoro-4-(trifluoromethyl)phenyl]-3-(pyridin-4-yl)-6,7-dihydropyrazolo[1,5-a]pyrazine-5(4H)-carboxylate (320 mg, 0.691 mmol, 65% yield) as yellow oil.

LC-MS (Method 16): Rₜ=0.817 min; MS (ESIpos): m/z=462.1 [M+H]⁺.

### Intermediate 203

### 2-[3-fluoro-4-(trifluoromethyl)phenyl]-3-(pyridin-4-yl)-4,5,6,7-tetrahydropyrazolo[1,5-a]pyrazine hydrochloride (1:1)

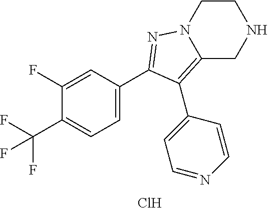 [SMILES: Fc1cc(-c2nn3c(c2-c2ccncc2)CNCC3)ccc1C(F)(F)F ; Cl]

To a solution of tert-butyl 2-[3-fluoro-4-(trifluoromethyl)phenyl]-3-(pyridin-4-yl)-6,7-dihydropyrazolo[1,5-a]pyrazine-5(4H)-carboxylate (320 mg, 0.692 mmol, Intermediate 202 in ethyl acetate (3.0 ml) was added hydrochloric acid (5.0 ml, 4M in ethyl acetate) at 20° C. The mixture was stirred at 20° C. for 2 hours. The reaction mixture was filtered and the solid cake was collected to give 2-[3-fluoro-4-(trifluoromethyl)phenyl]-3-(pyridin-4-yl)-4,5,6,7-tetrahydropyrazolo[1,5-a]pyrazine hydrochloride (1:1) (200 mg, 0.501 mmol, 72% yield) as a yellow solid.

LC-MS (Method 16): Rₜ=0.655 min; MS (ESIpos): m/z=362.1 [M+H]⁺.

### Intermediate 204

### tert-butyl 2-[2-fluoro-4-(trifluoromethyl)phenyl]-3-(pyridin-4-yl)-6,7-dihydropyrazolo[1,5-a]pyrazine-5(4H)-carboxylate

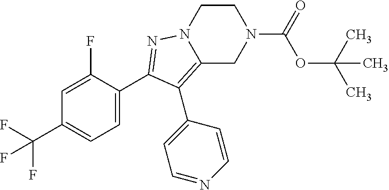 [SMILES: CC(C)(C)OC(=O)N1CCn2nc(-c3ccc(C(F)(F)F)cc3F)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and [2-fluoro-4-(trifluoromethyl)phenyl]boronic acid (329 mg, 1.58 mmol) in 1,4-dioxane (8.0 ml) and water (2.0 ml) were added (1,1′-bis(diphenylphosphino) ferrocene) palladium (II) dichloride (77.2 mg, 0.105 mmol) and sodium carbonate (224 mg, 2.11 mmol) at 20° C. The mixture was degassed with nitrogen and stirred at 90° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl acetate=0:1 to 3:1) to give tert-butyl 2-[2-fluoro-4-(trifluoromethyl)phenyl]-3-(pyridin-4-yl)-6,7-dihydropyrazolo[1,5-a]pyrazine-5(4H)-carboxylate (390 mg, 0.843 mmol, 79% yield) as a yellow oil.

LC-MS (Method 16): Rₜ=0.554 min; MS (ESIpos): m/z=462.1 [M+H]⁺.

### Intermediate 205

### 2-[2-fluoro-4-(trifluoromethyl)phenyl]-3-(pyridin-4-yl)-4,5,6,7-tetrahydropyrazolo[1,5-a]pyrazine hydrochloride (1:1)

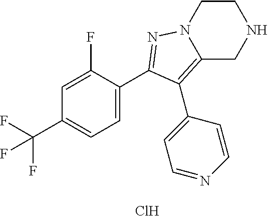 [SMILES: Fc1cc(C(F)(F)F)ccc1-c1nn2c(c1-c1ccncc1)CNCC2 ; Cl]

To a solution of tert-butyl 2-[2-fluoro-4-(trifluoromethyl)phenyl]-3-(pyridin-4-yl)-6,7-dihydropyrazolo[1,5-a]pyrazine-5(4H)-carboxylate (390 mg, 0.843 mmol, Intermediate 204) in ethyl acetate (2.8 ml) was added hydrochloric acid (4.6 ml, 4M in ethyl acetate) at 20° C. The mixture was stirred at 20° C. for 2 hours. The reaction mixture was filtered and washed with ethyl acetate. The solid cake was collected to give 2-[2-fluoro-4-(trifluoromethyl)phenyl]-3-(pyridin-4-yl)-4,5,6,7-tetrahydropyrazolo[1,5-a]pyrazine hydrochloride (1:1) (300 mg, 0.752 mmol, 89% yield) as a yellow solid.

LC-MS (Method 16): Rₜ=0.469 min; MS (ESIpos): m/z=362.1 [M+H]⁺

### Intermediate 206

### tert-butyl 2-(2-fluorophenyl)-3-(pyridin-4-yl)-6,7-dihydropyrazolo[1,5-a]pyrazine-5(4H)-carboxylate

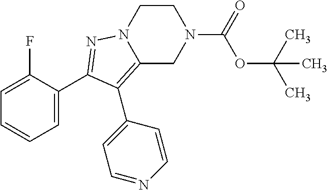 [SMILES: CC(C)(C)OC(=O)N1CCn2nc(-c3ccccc3F)c(-c3ccncc3)c2C1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and (2-fluorophenyl) boronic acid (166 mg, 1.19 mmol) in 1,4-dioxane (6 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 20° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(2-fluorophenyl)-3-(pyridin-4-yl)-6,7-dihydropyrazolo[1,5-a]pyrazine-5(4H)-carboxylate (300 mg, 761 μmol, 96% yield) as a brown oil.

LC-MS (Method 16): Rₜ=0.822 min; MS (ESIpos): m/z=395.2 [M+H]⁺.

### Intermediate 207

### 2-(2-fluorophenyl)-3-(pyridin-4-yl)-4,5,6,7-tetrahydropyrazolo[1,5-a]pyrazine hydrogen chloride (1:1)

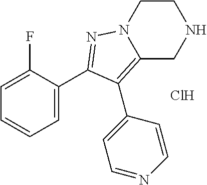 [SMILES: Fc1ccccc1-c1nn2c(c1-c1ccncc1)CNCC2 ; Cl]

To a solution of tert-butyl 2-(2-fluorophenyl)-3-(pyridin-4-yl)-6,7-dihydropyrazolo[1,5-a]pyrazine-5(4H)-carboxylate (300 mg, 761 μmol, Intermediate 206) in 1,4-dioxane (10 ml) was added hydrochloric acid (555 mg, 15.2 mmol, 4M in 1,4-dioxane) at 20° C. The mixture was stirred at 20° C. for 1 hour. The reaction mixture was concentrated under vacuum to give 2-(2-fluorophenyl)-3-(pyridin-4-yl)-4,5,6,7-tetrahydropyrazolo[1,5-a]pyrazine hydrogen chloride (1:1) (250 mg, 758 μmol, 99% yield) as a yellow oil.

### Intermediate 208

### tert-butyl 2-(2-chlorophenyl)-3-(pyridin-4-yl)-6,7-dihydropyrazolo[1,5-a]pyrazine-5(4H)-carboxylate

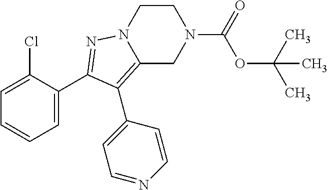 [SMILES: CC(C)(C)OC(=O)N1CCn2nc(-c3ccccc3Cl)c(-c3ccncc3)c2C1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and (2-chlorophenyl) boronic acid (148 mg, 949 μmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C., the reaction mixture was degassed with nitrogen, and then stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(2-chlorophenyl)-3-(pyridin-4-yl)-6,7-dihydropyrazolo[1,5-a]pyrazine-5(4H)-carboxylate (300 mg, 730 μmol, 92% yield) as a yellow oil.

LC-MS (Method 16): Rₜ=0.832 min; MS (ESIpos): m/z=411.2 [M+H]⁺.

### Intermediate 209

### 2-(2-chlorophenyl)-3-(pyridin-4-yl)-4,5,6,7-tetrahydropyrazolo[1,5-a]pyrazine hydrogen chloride (1:1)

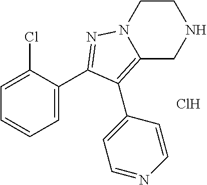 [SMILES: Clc1ccccc1-c1nn2c(c1-c1ccncc1)CNCC2 ; Cl]

To a solution of tert-butyl 2-(2-chlorophenyl)-3-(pyridin-4-yl)-6,7-dihydropyrazolo[1,5-a]pyrazine-5(4H)-carboxylate (300 mg, 730 μmol, Intermediate 208) in ethyl acetate (10 ml) was added hydrochloric acid (532 mg, 14.6 mmol, 4M in ethyl acetate) at 25° C. The mixture was stirred at 25° C. for 1 hour. The reaction mixture was concentrated under vacuum to give 2-(2-chlorophenyl)-3-(pyridin-4-yl)-4,5,6,7-tetrahydropyrazolo[1,5-a]pyrazine hydrogen chloride (1:1) (200 mg, 576 μmol, 79% yield) as a brown solid.

LC-MS (Method 14): Rₜ=0.300 min; MS (ESIpos): m/z=311.1 [M+H]⁺.

### Intermediate 210

### tert-butyl 2-(3-methoxyphenyl)-3-(pyridin-4-yl)-6,7-dihydropyrazolo[1,5-a]pyrazine-5(4H)-carboxylate

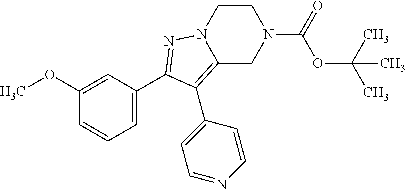 [SMILES: COc1cccc(-c2nn3c(c2-c2ccncc2)CN(C(=O)OC(C)(C)C)CC3)c1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and (3-methoxyphenyl) boronic acid (240 mg, 1.58 mmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 0:1) to give tert-butyl 2-(3-methoxyphenyl)-3-(pyridin-4-yl)-6,7-dihydropyrazolo[1,5-a]pyrazine-5(4H)-carboxylate (300 mg, 791 μmol, 93% yield) as a brown oil.

LC-MS (Method 16): Rₜ=0.821 min; MS (ESIpos): m/z=407.2 [M+H]⁺.

### Intermediate 211

### 2-(3-methoxyphenyl)-3-(pyridin-4-yl)-4,5,6,7-tetrahydropyrazolo[1,5-a]pyrazine hydrogen chloride (1:1)

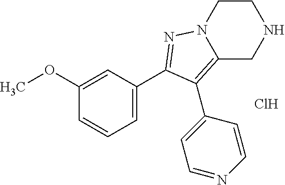 [SMILES: COc1cccc(-c2nn3c(c2-c2ccncc2)CNCC3)c1 ; Cl]

To a solution of tert-butyl 2-(3-methoxyphenyl)-3-(pyridin-4-yl)-6,7-dihydropyrazolo[1,5-a]pyrazine-5(4H)-carboxylate (300 mg, 738 μmol, Intermediate 210) in ethyl acetate (5 ml) was added hydrochloric acid (5.0 ml, 20.0 mmol, 4.0 M in ethyl acetate) at 25° C. The mixture was stirred at 25° C. for 1 hour. The reaction mixture was concentrated under vacuum to give 2-(3-methoxyphenyl)-3-(pyridin-4-yl)-4,5,6,7-tetrahydropyrazolo[1,5-a]pyrazine hydrogen chloride (1:1) (200 mg, 584 μmol, 79% yield) as a yellow solid.

LC-MS (Method 14): Rₜ=0.237 min; MS (ESIpos): m/z=307.1 [M+H]⁺.

### Intermediate 212

### tert-butyl 2-(4-methylphenyl)-3-(pyridin-4-yl)-6,7-dihydropyrazolo[1,5-a]pyrazine-5(4H)-carboxylate

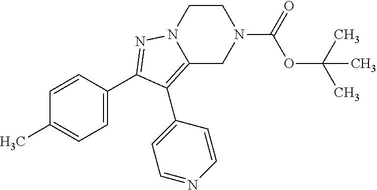 [SMILES: Cc1ccc(-c2nn3c(c2-c2ccncc2)CN(C(=O)OC(C)(C)C)CC3)cc1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and (4-methylphenyl) boronic acid (215 mg, 1.58 mmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 0:1) to give tert-butyl 2-(4-methylphenyl)-3-(pyridin-4-yl)-6,7-dihydropyrazolo[1,5-a]pyrazine-5(4H)-carboxylate (280 mg, 717 μmol, 91% yield) as a brown oil.

LC-MS (Method 16): Rₜ=0.835 min; MS (ESIpos): m/z=391.2 [M+H]⁺.

### Intermediate 213

### 2-(4-methylphenyl)-3-(pyridin-4-yl)-4,5,6,7-tetrahydropyrazolo[1,5-a]pyrazine hydrogen chloride (1:1)

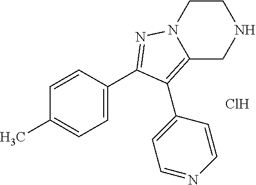 [SMILES: Cc1ccc(-c2nn3c(c2-c2ccncc2)CNCC3)cc1 ; Cl]

To a solution of tert-butyl 2-(4-methylphenyl)-3-(pyridin-4-yl)-6,7-dihydropyrazolo[1,5-a]pyrazine-5(4H)-carboxylate (280 mg, 717 μmol, Intermediate 212) in ethyl acetate (5 ml) was added hydrochloric acid (4.7 ml, 19.0 mmol, 4.0 M in ethyl acetate) at 25° C. The mixture was stirred at 25° C. for 1 hour. The reaction mixture was concentrated under vacuum to give 2-(4-methylphenyl)-3-(pyridin-4-yl)-4,5,6,7-tetrahydropyrazolo[1,5-a]pyrazine hydrogen chloride (1:1) (210 mg, 643 μmol, 90% yield) as a yellow solid.

LC-MS (Method 20): Rₜ=0.481 min; MS (ESIpos): m/z=291.4 [M+H]⁺.

### Intermediate 214

### tert-butyl 2-(3-methylphenyl)-3-(pyridin-4-yl)-6,7-dihydropyrazolo[1,5-a]pyrazine-5(4H)-carboxylate

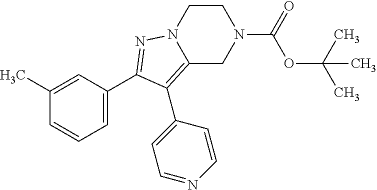 [SMILES: Cc1cccc(-c2nn3c(c2-c2ccncc2)CN(C(=O)OC(C)(C)C)CC3)c1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and 3-methylphenyl) boronic acid (215 mg, 1.58 mmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 0:1) to give tert-butyl 2-(3-methylphenyl)-3-(pyridin-4-yl)-6,7-dihydropyrazolo[1,5-a]pyrazine-5(4H)-carboxylate (290 mg, 743 μmol, 94% yield) as a brown oil.

LC-MS (Method 16): Rₜ=0.847 min; MS (ESIpos): m/z=391.2 [M+H]⁺.

### Intermediate 215

### 2-(3-methylphenyl)-3-(pyridin-4-yl)-4,5,6,7-tetrahydropyrazolo[1,5-a]pyrazine hydrogen chloride (1:1)

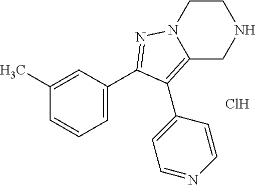 [SMILES: Cc1cccc(-c2nn3c(c2-c2ccncc2)CNCC3)c1 ; Cl]

To a solution of tert-butyl 2-(3-methylphenyl)-3-(pyridin-4-yl)-6,7-dihydropyrazolo[1,5-a]pyrazine-5(4H)-carboxylate (290 mg, 743 μmol, Intermediate 214) in ethyl acetate (5 ml) was added hydrochloric acid (5.0 ml, 20.0 mmol, 4.0 M in ethyl acetate) at 25° C. The mixture was stirred at 25° C. for 1 hour. The reaction mixture was concentrated under vacuum to give 2-(3-methylphenyl)-3-(pyridin-4-yl)-4,5,6,7-tetrahydropyrazolo[1,5-a]pyrazine hydrogen chloride (1:1) (220 mg, 673 μmol, 91% yield) as a yellow solid.

LC-MS (Method 20): Rₜ=0.483 min; MS (ESIpos): m/z=291.4 [M+H]⁺.

### Intermediate 216

### tert-butyl 2-(4-ethylphenyl)-3-(pyridin-4-yl)-6,7-dihydropyrazolo[1,5-a]pyrazine-5(4H)-carboxylate

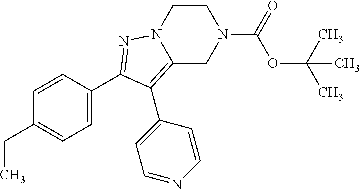 [SMILES: CCc1ccc(-c2nn3c(c2-c2ccncc2)CN(C(=O)OC(C)(C)C)CC3)cc1]

To a solution of tert-butyl 2-bromo-3-(pyridin-4-yl)-6,7-dihydropyrazolo[1,5-a]pyrazine-5(4H)-carboxylate (500 mg, 1.32 mmol, Intermediate 68) and (4-ethylphenyl) boronic acid (297 mg, 1.98 mmol) in 1,4-dioxane (10.0 ml) and water (3.0 ml) were added (1,1′-bis(diphenylphosphino) ferrocene) palladium (II) dichloride (96.5 mg, 0.132 mmol) and sodium carbonate (279 mg, 2.64 mmol) at 20° C. The mixture was stirred at 90° C. for 16 hours under nitrogen atmosphere. The reaction mixture was poured into water and extracted with ethyl acetate. The organic phase was concentrated in vacuo to give a crude product. The crude was purified by silica gel column chromatography (petroleum ether:ethyl acetate=50:1 to 1:1) to give tert-butyl 2-(4-ethylphenyl)-3-(pyridin-4-yl)-6,7-dihydropyrazolo[1,5-a]pyrazine-5(4H)-carboxylate (320 mg, 791 μmol, 60% yield) as a yellow solid.

LC-MS (Method 16): Rₜ=0.827 min; MS (ESIpos): m/z=405.3 [M+H]⁺.

### Intermediate 217

### 2-(4-ethylphenyl)-3-(pyridin-4-yl)-4,5,6,7-tetrahydropyrazolo[1,5-a]pyrazine hydrochloride (1:1)

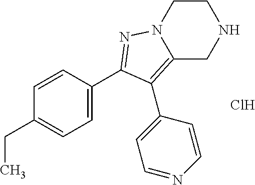 [SMILES: CCc1ccc(-c2nn3c(c2-c2ccncc2)CNCC3)cc1 ; Cl]

To a solution of tert-butyl 2-(4-ethylphenyl)-3-(pyridin-4-yl)-6,7-dihydropyrazolo[1,5-a]pyrazine-5(4H)-carboxylate (320 mg, 791 μmol, Intermediate 216) in ethyl acetate (5.0 ml) was added hydrochloric acid (2.0 ml, 4 M in ethyl acetate) at 25° C. The mixture was stirred at 25° C. for 0.5 hour. The reaction mixture was concentrated in vacuo to give 2-(4-ethylphenyl)-3-(pyridin-4-yl)-4,5,6,7-tetrahydropyrazolo[1,5-a]pyrazine hydrochloride (1:1) (260 mg, 763 mmol, 96% yield) as a brown solid.

### Intermediate 218

### tert-butyl 2-(3-cyanophenyl)-3-(pyridin-4-yl)-6,7-dihydropyrazolo[1,5-a]pyrazine-5(4H)-carboxylate

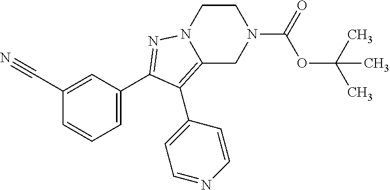 [SMILES: CC(C)(C)OC(=O)N1CCn2nc(-c3cccc(C#N)c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and (3-cyanophenyl) boronic acid (232 mg, 1.58 mmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(3-cyanophenyl)-3-(pyridin-4-yl)-6,7-dihydropyrazolo[1,5-a]pyrazine-5(4H)-carboxylate (160 mg, 399 μmol, 50% yield) as a yellow oil.

LC-MS (Method 16): Rₜ=0.798 min; MS (ESIpos): m/z=402.2 [M+H]⁺.

### Intermediate 219

### 3-[3-(pyridin-4-yl)-4,5,6,7-tetrahydropyrazolo[1,5-a]pyrazin-2-yl]benzonitrile hydrogen chloride (1:1)

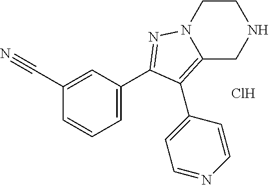 [SMILES: N#Cc1cccc(-c2nn3c(c2-c2ccncc2)CNCC3)c1 ; Cl]

To a solution of tert-butyl 2-(3-cyanophenyl)-3-(pyridin-4-yl)-6,7-dihydropyrazolo[1,5-a]pyrazine-5(4H)-carboxylate (160 mg, 399 μmol, Intermediate 218) in ethyl acetate (5 ml) was added hydrochloric acid (4.0 ml, 16 mmol, 4.0 M in ethyl acetate) at 25° C. The mixture was stirred at 25° C. for 1 hour. The reaction mixture was concentrated under vacuum to give 3-[3-(pyridin-4-yl)-4,5,6,7-tetrahydropyrazolo[1,5-a]pyrazin-2-yl]benzonitrile hydrogen chloride (1:1) (130 mg, 385 μmol, 97% yield) as a yellow solid.

LC-MS (Method 20): Rₜ=0.795 min; MS (ESIpos): m/z=302.1 [M+H]⁺.

### Intermediate 220

### tert-butyl 2-(2-fluoro-4-methoxyphenyl)-3-(pyridin-4-yl)-6,7-dihydropyrazolo[1,5-a]pyrazine-5(4H)-carboxylate

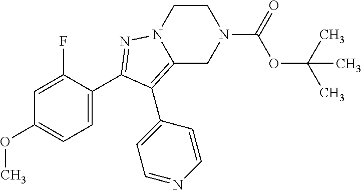 [SMILES: COc1ccc(-c2nn3c(c2-c2ccncc2)CN(C(=O)OC(C)(C)C)CC3)c(F)c1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate and (2-fluoro-4-methoxyphenyl) boronic acid (269 mg, 1.58 mmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(2-fluoro-4-methoxyphenyl)-3-(pyridin-4-yl)-6,7-dihydropyrazolo[1,5-a]pyrazine-5(4H)-carboxylate (310 mg, 730 μmol, 92% yield) as a yellow oil.

LC-MS (Method 16): Rₜ=0.809 min; MS (ESIpos): m/z=425.2 [M+H]⁺

### Intermediate 221

### 2-(2-fluoro-4-methoxyphenyl)-3-(pyridin-4-yl)-4,5,6,7-tetrahydropyrazolo[1,5-a]pyrazine hydrogen chloride (1:1)

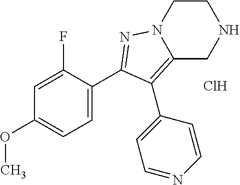 [SMILES: COc1ccc(-c2nn3c(c2-c2ccncc2)CNCC3)c(F)c1 ; Cl]

To a solution of tert-butyl 2-(2-fluoro-4-methoxyphenyl)-3-(pyridin-4-yl)-6,7-dihydropyrazolo[1,5-a]pyrazine-5(4H)-carboxylate (310 mg, 730 μmol, Intermediate 220) in ethyl acetate (5 ml) was added hydrochloric acid (5.0 ml, 20 mmol, 4.0 M in ethyl acetate) at 25° C. The mixture was stirred at 25° C. for 1 hour. The reaction mixture was concentrated under vacuum to give 2-(2-fluoro-4-methoxyphenyl)-3-(pyridin-4-yl)-4,5,6,7-tetrahydropyrazolo[1,5-a]pyrazine hydrogen chloride (1:1) (240 mg, 666 μmol, 91% yield) as a yellow solid.

LC-MS (Method 20): Rₜ=0.809 min; MS (ESIpos): m/z=325.1 [M+H]⁺.

### Intermediate 222

### tert-butyl 3-(pyridin-4-yl)-2-(4-((trifluoromethyl)thio)phenyl)-6,7-dihydropyrazolo[1,5-a]pyrazine-5(4H)-carboxylate

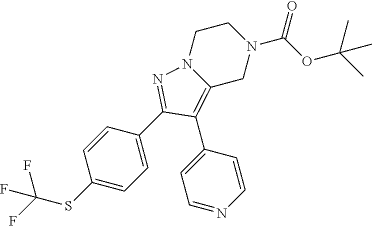 [SMILES: CC(C)(C)OC(=O)N1CCn2nc(-c3ccc(SC(F)(F)F)cc3)c(-c3ccncc3)c2C1]

To a solution of tert-butyl 2-bromo-3-(pyridin-4-yl)-6,7-dihydropyrazolo[1,5-a]pyrazine-5(4H)-carboxylate (500 mg, 13.2 mmol Intermediate 68) and 4,4,5,5-tetramethyl-2-(4-((trifluoromethyl)thio)phenyl)-1,3,2-dioxaborolane (802 mg, 26.4 mmol) in 1,4-dioxane (4 ml) and water (1 ml) were added sodium carbonate (350 mg, 33.0 mmol) and (1,1′-bis(diphenylphosphino) ferrocene) palladium (II) dichloride (96.0 mg, 1.31 mmol) at 25° C. The mixture was stirred at 100° C. for 16 hours under nitrogen atmosphere. The reaction mixture was concentrated under reduced pressure to give a residue. The residue was purified by column chromatography on silica gel (100~200 mesh, petreoleum ether:ethyl acetate=10:1 to 1:1) to give tert-butyl 3-(pyridin-4-yl)-2-(4-((trifluoromethyl)thio)phenyl)-6,7-dihydropyrazolo[1,5-a]pyrazine-5(4H)-carboxylate (350 mg, 94% purity, 52% yield) as a yellow oil.

LC-MS (Method 16): Rₜ=0.548 min; MS (ESIpos): m/z=477.1 [M+H]⁺.

Intermediate 223 3-(pyridin-4-yl)-2-(4-((trifluoromethyl)thio)phenyl)-4,5,6,7-tetrahydropyrazolo[1,5-a]pyrazine hydrochloride (1:1)

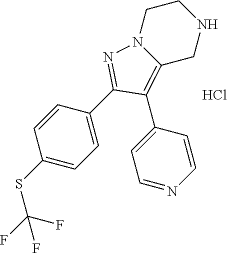 [SMILES: FC(F)(F)Sc1ccc(-c2nn3c(c2-c2ccncc2)CNCC3)cc1 ; Cl]

The solution of tert-butyl 3-(pyridin-4-yl)-2-(4-((trifluoromethyl)thio)phenyl)-6,7-dihydropyrazolo[1,5-a]pyrazine-5(4H)-carboxylate (350 mg, 94% purity, 0.690 mmol Intermediate 222) in hydrochloric acid (4 ml, 4M in ethyl acetate) was stirred at 25° C. for 2 hours. The mixture was concentrated under reduced pressure to give 3-(pyridin-4-yl)-2-(4-((trifluoromethyl)thio)phenyl)-4,5,6,7-tetrahydropyrazolo[1,5-a]pyrazine hydrochloride (1:1) (300 mg, 82% purity, 86% yield) as a colorless oil which was used in the next step without purification.

LC-MS (Method 15): Rₜ=0.821 min; MS (ESIpos): m/z=377.1 [M+H]⁺

### Intermediate 224

### tert-butyl 2-(4-ethynylphenyl)-3-(pyridin-4-yl)-6,7-dihydropyrazolo[1,5-a]pyrazine-5(4H)-carboxylate

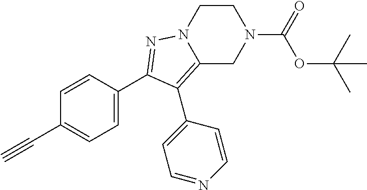 [SMILES: C#Cc1ccc(-c2nn3c(c2-c2ccncc2)CN(C(=O)OC(C)(C)C)CC3)cc1]

To a solution of tert-butyl 2-bromo-3-(pyridin-4-yl)-6,7-dihydropyrazolo[1,5-a]pyrazine-5(4H)-carboxylate (200 mg, 0.527 mmol, Intermediate 68) and trimethyl {[4-(4,4,5,5-tetramethyl-1,3,2-dioxaborolan-2-yl)phenyl]ethynyl}silane (237 mg, 0.791 mmol) in 1,4-dioxane (4 ml) and ml) were added water (1 (1,1′-bis(diphenylphosphino) ferrocene) palladium (II) dichloride (38.5 mg, 0.0527 mmol) and sodium carbonate (111 mg, 1.05 mmol) at 20° C. The mixture was degassed with nitrogen and stirred at 90° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative TLC (petroleum ether:ethyl acetate=1:0 to 0:1) to give tert-butyl 2-(4-ethynylphenyl)-3-(pyridin-4-yl)-6,7-dihydropyrazolo[1,5-a]pyrazine-5(4H)-carboxylate (140 mg, 0.349 mmol, 66% yield) as a yellow oil.

LC-MS (Method 16): Rₜ=0.808 min; MS (ESIpos): m/z=400.1 [M+H]⁺.

### Intermediate 225

### 2-(4-ethynylphenyl)-3-(pyridin-4-yl)-4,5,6,7-tetrahydropyrazolo[1,5-a]pyrazine trifluoroacetate (1:1)

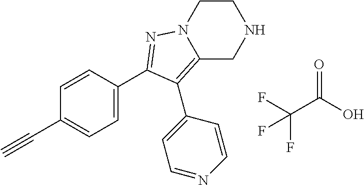

To a solution of tert-butyl 2-(4-ethynylphenyl)-3-(pyridin-4-yl)-6,7-dihydropyrazolo[1,5-a]pyrazine-5(4H)-carboxylate (140 mg, 0.149 mmol, Intermediate 224) in dichloromethane (5 ml) was added trifluoroacetic acid (3 ml) at 20° C. The mixture was stirred at 20° C. for 1 hour. The reaction mixture was concentrated in vacuo to give 2-(4-ethynylphenyl)-3-(pyridin-4-yl)-4,5,6,7-tetrahydropyrazolo[1,5-a]pyrazine trifluoroacetate (1:1) (90.0 mg, 0.217 mmol, 62% yield) as a brown oil.

LC-MS (Method 16): Rₜ=0.446 min; MS (ESIpos): m/z=300.1 [M+H]⁺.

### Intermediate 226

### methyl(2E)-4-(azetidin-1-yl) but-2-enoate

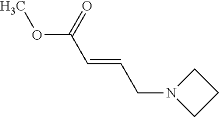

To a solution of methyl(2E)-4-bromobut-2-enoate (1.00 g, 5.59 mmol) and azetidine (0.638 g. 11.2 mmol) in tetrahydrofuran (15 ml) was added triethylamine (1.13 g, 11.2 mmol) at 20° C. The mixture was stirred at 20° C. for 15 hours. The reaction mixture was filtered and the filtrate was concentrated under reduced pressure to give a residue. The residue was purified by silica gel column chromatography (petroleum ether:ethyl acetate=1:1 to 1:2) to give methyl(2E)-4-(azetidin-1-yl) but-2-enoate (0.450 g, 2.90 mmol, 52% yield) as a yellow oil. 1H NMR (400 MHz, DMSO-d₆), δ [ppm]=6.73 (d, J=15.6, 5.2 Hz, 1H), 5.91 (d, J=15.6, 1.6 Hz, 1H), 3.64 (s, 3H), 3.15-3.09 (m, 6H), 1.99 (s, 2H).

### Intermediate 227

### (2E)-4-(azetidin-1-yl) but-2-enoic acid

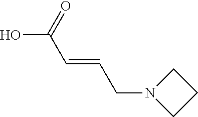

To a solution of methyl(2E)-4-(azetidin-1-yl) but-2-enoate (450 mg, 2.90 mmol, Intermediate 2) in a mixed solvent of tetrahydrofuran (10 ml) and water (5 ml) was added lithium hydroxide (174 mg, 7.25 mmol) at 20° C. The mixture was stirred at 50° C. for 15 hours. pH of the reaction mixture was adjusted to 6 with hydrochloric acid (1 M in water) and the mixture was concentrated under reduced pressure to give (2E)-4-(azetidin-1-yl) but-2-enoic acid (400 mg, 2.83 mmol, 98% yield).

¹H NMR (400 MHz, DMSO-d₆) δ [ppm]=6.45-6.31 (m, 1H), 5.78 (d, J=15.6 Hz, 1H), 3.17-3.00 (m, 6H), 2.06-1.89 (m, 2H).

### Intermediate 228

### methyl(2E)-4-(piperidin-1-yl) but-2-enoate

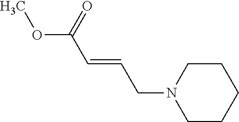

To a solution of methyl(2E)-4-bromobut-2-enoate (2.00 g, 11.2 mmol) and piperidine (1.90 g, 22.3 mmol) in tetrahydrofuran (30 ml) was added trimethylamine (2.26 g, 22.3 mmol) at 20° C. The mixture was stirred at 20° C. for 15 hours. The reaction mixture was filtered and the filtrate was concentrated under reduced pressure to give methyl(2E)-4-(piperidin-1-yl) but-2-enoate (1.99 g, 10.8 mmol, 97% yield) as a yellow oil.

¹H NMR (400 MHz, DMSO-d₆), δ [ppm]=6.82 (td, J=15.6, 6.0 Hz, 1H), 5.98 (td, J=15.6, 1.6 Hz, 1H), 3.65 (s, 3H), 3.05 (dd, J=6.0, 1.6 Hz, 2H), 2.30 (br s, 4H), 1.52-1.44 (m, 4H), 1.40-1.30 (m, 2H).

### Intermediate 229

### (2E)-4-(piperidin-1-yl) but-2-enoic acid

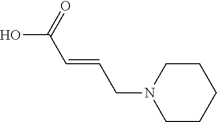 [SMILES: O=C(O)/C=C/CN1CCCCC1]

To a solution of methyl(2E)-4-(piperidin-1-yl) but-2-enoate (300 mg, 1.64 mmol, Intermediate 228) in a mixed solvent of methanol (6 ml) and water (3 ml) was added lithium hydroxide (98.0 mg, 4.09 mmol) at 20° C. The mixture was stirred at 50° C. for 15 hours. pH of the reaction mixture was adjusted to 6 with hydrochloric acid (1 M in water). The mixture was concentrated under reduced pressure to give (2E)-4-(piperidin-1-yl) but-2-enoic acid (270 mg, 1.60 mmol, 97% yield) as a white solid.

¹H NMR (400 MHz, DMSO-d6) δ [ppm]=6.72 (td, J=15.6, 6.4 Hz, 1H), 5.94 (d, J=15.6 Hz, 1H), 3.19 (d, J=6.0 Hz, 2H), 2.46 (br s, 4H), 1.58-1.30 (m, 6H).

### Intermediate 230

### methyl(2E)-4-(4-methylpiperazin-1-yl) but-2-enoate

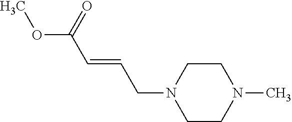

To a solution of methyl(2E)-4-bromobut-2-enoate (1.00 g, 5.59 mmol) and 1-methylpiperazine (1.12 g, 11.2 mmol) in tetrahydrofuran (15 ml) was added triethylamine (1.13 g, 11.2 mmol) at 20° C. The mixture was stirred at 20° C. for 15 hours. The reaction mixture was filtered and the filtrate was concentrated under reduced pressure to give methyl (2E)-4-(4-methylpiperazin-1-yl) but-2-enoate (1.00 g, 5.04 mmol, 90% yield) as a yellow oil.

LC-MS (Method 20): Rₜ=0.602 min; MS (ESIpos): m/z=199.2 [M+H]⁺

### Intermediate 231

### (2E)-4-(4-methylpiperazin-1-yl) but-2-enoic acid

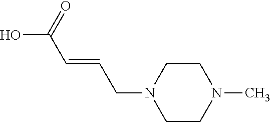

To a solution of methyl(2E)-4-(4-methylpiperazin-1-yl) but-2-enoate (500 mg, 2.52 mmol, Intermediate 230) in a mixed solvent of methanol (10 ml) and water (2.5 ml) was added sodium hydroxide (303 mg, 7.57 mmol) at 20° C. The mixture was stirred at 20° C. for 15 hours. pH of reaction mixture was adjusted to 6 with hydrochloric acid (1M in water) and the mixture was lyophilized to give a crude product. The crude product was triturated with methanol to give (2E)-4-(4-methylpiperazin-1-yl) but-2-enoic acid (400 mg, 2.17 mmol, 86% yield) as a white solid.

LC-MS (Method 16): Rₜ=0.130 min; MS (ESIpos): m/z=185.1 [M+H]⁺.

### Intermediate 232

### tert-butyl 2-(4-chlorophenyl)-3-(pyridin-4-yl)-6,7-dihydropyrazolo[1,5-a]pyrazine-5(4H)-carboxylate

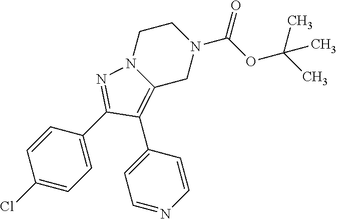 [SMILES: CC(C)(C)OC(=O)N1CCn2nc(-c3ccc(Cl)cc3)c(-c3ccncc3)c2C1]

To a solution of tert-butyl 2-bromo-3-(pyridin-4-yl)-6,7-dihydropyrazolo[1,5-a]pyrazine-5(4H)-carboxylate (3.00 g, 7.91 mmol, Intermediate 68) and (4-chlorophenyl) boronic acid (2.47 g, 15.8 mmol) in 1,4-dioxane (48 ml) and water (12 ml) were added sodium carbonate (1.68 g, 15.8 g) and [1,1-Bis(diphenylphosphino) ferrocene]palladium (II) chloride (0.579 g, 0.791 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 1:1) to give a crude product. The crude product was triturated with a mixed solvent of petroleum ether and ethyl aceate (1:1, v/v) to give tert-butyl 2-(4-chlorophenyl)-3-(pyridin-4-yl)-6,7-dihydropyrazolo[1,5-a]pyrazine-5(4H)-carboxylate (2.00 g, 4.87 mmol, 62% yield) as a white solid.

LC-MS (Method 16): Rₜ=0.504 min; MS (ESIpos): m/z=411.1 [M+H]⁺.

### Intermediate 233

### 2-(4-chlorophenyl)-3-(pyridin-4-yl)-4,5,6,7-tetrahydropyrazolo[1,5-a]pyrazine hydrochloride (1:1)

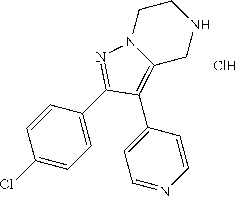 [SMILES: Clc1ccc(-c2nn3c(c2-c2ccncc2)CNCC3)cc1 ; Cl]

To a solution of tert-butyl 2-(4-chlorophenyl)-3-(pyridin-4-yl)-6,7-dihydropyrazolo[1,5-a]pyrazine-5(4H)-carboxylate (2.00 g, 4.87 mmol, Intermediate 232) in ethyl acetate (18 ml) was added hydrochloric acid (4M in ethyl acetate, 12 ml) at 20° C. The mixture was stirred at 20° C. for 2 hours. The reaction mixture was filtered and the solid cake was collected to give 2-(4-chlorophenyl)-3-(pyridin-4-yl)-4,5,6,7-tetrahydropyrazolo[1,5-a]pyrazine hydrochloride (1:1) (1.80 g, 5.18 mmol, 107% yield) as a white solid.

LC-MS (Method 16): Rₜ=0.435 min; MS (ESIpos): m/z=311.1 [(M−36)+H]⁺.

### Intermediate 234

### methyl(2E)-4-[isopropyl(methyl)amino]but-2-enoate

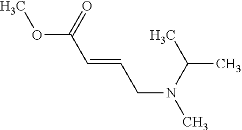

To a solution of methyl(2E)-4-bromobut-2-enoate (1.00 g, 5.59 mmol) and N-methylpropan-2-amine (0.817 g, 11.2 mmol) in tetrahydrofuran (20 ml) was added triethylamine (1.13 g, 11.2 mmol) at 20° C. The mixture was stirred at 20° C. for 3 hours. The reaction mixture was filtered and the filtrate was concentrated under reduced pressure to give methyl(2E)-4-[isopropyl(methyl)amino]but-2-enoate (700 mg, 4.09 mmol, 73% yield) as a yellow oil.

¹H NMR (400 MHz, DMSO-d6), δ [ppm]=6.82 (td, J=15.6, 5.6 Hz, 1H), 6.00 (td, J=15.6, 1.6 Hz, 1H), 3.65 (s, 3H), 3.13 (dd, J=5.6, 1.6 Hz, 2H), 2.76 (td, J=13.2, 6.4 Hz, 1H), 2.08 (s, 3H), 0.94 (d, J=6.8 Hz, 6H).

### Intermediate 235

### (2E)-4-[isopropyl(methyl)amino]but-2-enoic acid

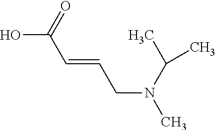

To a solution of methyl(2E)-4-[isopropyl(methyl)amino]but-2-enoate (400 mg, 2.34 mmol, Intermediate 234) in methanol (3 ml) and water (3 ml) was added sodium hydroxide (280 mg, 7.01 mmol) at 20° C. The mixture was stirred at 50° C. for 15 hours. pH of the reaction mixture was adjusted to 6 with hydrochloric acid (1 M in water) and the resulting mixture was concentrated under reduced pressure to give (2E)-4-[isopropyl(methyl)amino]but-2-enoic acid (350 mg, 2.23 mmol, 95% yield) as a white solid.

¹H NMR (400 MHz, DMSO-d₆), δ [ppm]=6.74 (td, J=15.6, 6.0, Hz 1H), 5.94 (d, J=15.6 Hz, 1H), 3.77 (td, J=12.4, 6.0, Hz 1H), 3.21 (d, J=6.0 Hz, 2H), 2.91-2.79 (m, 1H), 2.14 (s, 3H), 0.98 (d, J=6.4 Hz, 6H).

### Intermediate 236

### methyl(2E)-4-(tert-butylamino)but-2-enoate

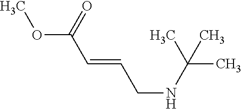

To a solution of methyl(2E)-4-bromobut-2-enoate (3.00 g, 16.8 mmol) and 2-methylpropan-2-amine (2.45 g, 33.5 mmol) in tetrahydrofuran (50 ml) was added triethylamine (3.39 g, 33.5 mmol) at 20° C. and the mixture was stirred at 20° C. for 15 hours. The reaction mixture was filtered and the filtrate was concentrated under reduced pressure to give methyl(2E)-4-(tert-butylamino)but-2-enoate (1.80 g, 10.5 mmol, 63% yield) as a yellow oil.

¹H NMR (400 MHz, DMSO-d6), δ [ppm]=6.94 (td, J=15.6, 5.2 Hz, 1H), 6.00 (td, J=15.6, 1.6 Hz, 1H), 3.64 (s, 3H), 3.29 (dd, J=5.2, 1.6 Hz, 2H), 1.02 (s, 9H).

### Intermediate 237

### (2E)-4-(tert-butylamino)but-2-enoic acid

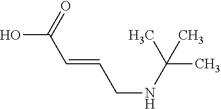

To a solution of methyl(2E)-4-(tert-butylamino)but-2-enoate (500 mg, 2.92 mmol, Intermediate 236) in a mixed solvent of tetrahydrofuran (12 ml) and water (3 ml) was added lithium hydroxide monohydrate (245 mg, 5.84 mmol) at 20° C. and the mixture was stirred at 20° C. for 15 hours. pH of reaction mixture was adjusted to 5~6 with hydrochloric acid (1M in water) and the resulting mixture was concentrated under reduced pressure to give a residue. To the residue was added ethanol and the resulting mixture was filtered. The filtrate was concentrated under vacuum to give a crude product. The crude product was triturated with a mixed solvent of propan-2-ol and ethyl acetate (6 ml, 1:5, v/v) to give (2E)-4-(tert-butylamino)but-2-enoic acid (140 mg, 0.890 mmol, 31% yield) as a white solid.

LC-MS (Method 15): Rₜ=0.129 min; MS (ESIpos): m/z=158.1 [M+H]⁺.

### Intermediate 238

### (2E)-4-[(2-hydroxyethyl)(methyl)amino]but-2-enoate

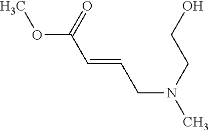

To a solution of methyl(2E)-4-bromobut-2-enoate (2.00 g, 11.2 mmol) and 2-(methylamino) ethanol (0.923 mg, 12.3 mmol) in tetrahydrofuran (40 ml) was added trimethylamine (1.13 g, 11.2 mmol) at 20° C. and the mixture stirred at 20° C. for 15 hours. The reaction mixture was filtered and the filtrate was concentrated under reduced pressure to give methyl(2E)-4-[(2-hydroxyethyl)(methyl)amino]but-2-enoate (1.90 g, 11.0 mmol, 98% yield) as a yellow oil.

¹H NMR (400 MHz, DMSO-d6), δ [ppm]=6.84 (td, J=15.6, 6.0 Hz, 1H), 6.09-5.95 (m, 1H), 4.41 (t, J=6.0 Hz, 1H), 3.66 (s, 3H), 3.50-3.40 (m, 2H), 3.16 (dd, J=6.0, 1.2 Hz, 2H), 2.40 (t, J=6.4 Hz, 2H), 2.17 (s, 3H).

### Intermediate 239

### N-(2-{[tert-butyl(dimethyl) silyl]oxy}ethyl)-N-methylpropan-1-amine

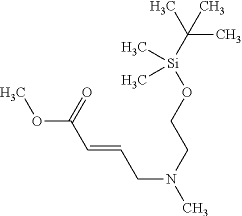 [SMILES: COC(=O)/C=C/CN(C)CCO[Si](C)(C)C(C)(C)C]

To a solution of methyl(2E)-4-[(2-hydroxyethyl)(methyl)amino]but-2-enoate (500 mg, 2.89 mmol, Intermediate 238) in dichloromethane (10 ml) was added 1H-imidazole (295 mg, 4.33 mmol), followed by addition of tert-butyl(chloro) dimethylsilane (653 mg, 4.33 mmol) at 0° C. The mixture was stirred at 20° C. for 3 hours. The reaction mixture was filtered and filtrate was concentrated under reduced pressure to give a residue. The residue was purified by silica gel column chromatography (petroleum ether:ethyl acetate=1:1 to ethyl acetate:methanol=4:1) to give N-(2-{[tert-butyl(dimethyl) silyl]oxy}ethyl)-N-methylpropan-1-amine (350 mg, 1.21 mmol, 42% yield) as a yellow oil.

LC-MS (Method 16): Rₜ=0.740 min; MS (ESIpos): m/z=288.2 [M+H]⁺.

### Intermediate 240

### (2E)-4-[(2-hydroxyethyl)(methyl)amino]but-2-enoic acid

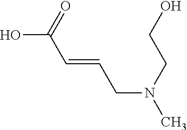

To a solution of methyl(2E)-4-[(2-{[tert-butyl(dimethyl) silyl]oxy}ethyl)(methyl)amino]but-2-enoate (350 mg, 1.22 mmol, Intermediate 239) in a mixed solvent of tetrahydrofuran (4 ml) and water (1 ml) was added lithium hydroxide monohydrate (102 mg, 2.43 mmol) at 20° C. and the mixture was stirred at 20° C. for 15 hours. pH of reaction mixture was adjusted to 6 with hydrochloric acid (1M in water) and the mixture was concentrated under reduced pressure to give (2E)-4-[(2-hydroxyethyl)(methyl)amino]but-2-enoic acid (300 mg, 1.88 mmol, 154% yield) as a yellow gum.

LC-MS (Method 16): Rₜ=0.100 min; MS (ESIpos): m/z=160.1 [M+H]⁺

### Intermediate 241

### methyl(2E)-4-(pyrrolidin-1-yl) but-2-enoate

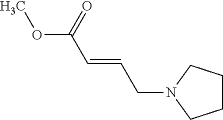

To a solution of methyl(2E)-4-bromobut-2-enoate (5.00 g, 27.9 mmol) and pyrrolidine (2.98 g. 41.9 mmol) in tetrahydrofuran (50 ml) was added triethylamine (2.83 g, 27.9 mmol) at 20° C. The mixture was stirred at 20° C. for 15 hours. The reaction mixture was concentrated under reduced pressure to give a residue. The residue was purified by silica gel column chromatography (petroleum ether:ethyl acetate=1:1 to dichloromethane:methanol=10:1) to give methyl(2E)-4-(pyrrolidin-1-yl) but-2-enoate (1.80 g, 10.6 mmol, 38% yield) as a yellow oil.

LC-MS (Method 20): Rₜ=0.771 min; MS (ESIpos): m/z=170.2 [M+H]⁺.

### Intermediate 242

### (2E)-4-(pyrrolidin-1-yl) but-2-enoic acid

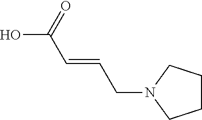 [SMILES: O=C(O)/C=C/CN1CCCC1]

To a solution of methyl(2E)-4-(pyrrolidin-1-yl) but-2-enoate (500 mg, 2.95 mmol, Intermediate 241) in a mixed solvent of tetrahydrofuran (10 ml) and water (5 ml) was added lithium hydroxide monohydrate (310 mg, 7.39 mmol) at 20° C. and the mixture was stirred at 20° C. for 15 hours. pH of the reaction mixture was adjusted to 6 with hydrochloric acid (1M in water) and the resulting solution was lyophilized to give a crude product. The crude product was triturated with ethanol to give (2E)-4-(pyrrolidin-1-yl) but-2-enoic acid (300 mg, 1.93 mmol, 65% yield) as a yellow solid.

¹H NMR (400 MHz, DMSO-d6), δ [ppm]=6.83 (td, J=15.6, 7.2 Hz, 1H), 6.19 (d, J=15.6 Hz, 1H), 3.94 (d, J=6.8 Hz, 2H), 3.16 (s, 4H), 1.90 (s, 4H).

### Intermediate 243

### methyl(2E)-4-(3-fluoroazetidin-1-yl) but-2-enoate

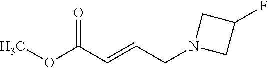

To a solution of methyl(2E)-4-bromobut-2-enoate (1.00 g, 5.59 mmol) and 3-fluoroazetidine (0.839 g, 11.2 mmol) in tetrahydrofuran (15 ml) was added N,N-diisopropylethylamine (1.44 g, 11.2 mmol) at 20° C. and the mixture was stirred at 20° C. for 15 hours. The reaction mixture was concentrated under reduced pressure to give a residue. The residue was purified by silica gel column chromatography (petroleum ether:ethyl acetate=1:0 to 2:1) to give methyl(2E)-4-(3-fluoroazetidin-1-yl) but-2-enoate (950 mg, 5.48 mmol, 98% yield) as a colorless oil.

LC-MS (Method 20): Rₜ=0.641 min; MS (ESIpos): m/z=174.2 [M+H]⁺

### Intermediate 244

### (2E)-4-(3-fluoroazetidin-1-yl) but-2-enoic acid

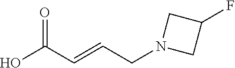 [SMILES: O=C(O)/C=C/CN1CC(F)C1]

To a solution of methyl(2E)-4-(3-fluoroazetidin-1-yl) but-2-enoate (500 mg, 2.68 mmol, Intermediate 243) in a mixed solvent of tetrahydrofuran (5 ml) and water (2 ml) was added lithium hydroxide monohydrate (242 mg, 5.77 mmol) at 20° C. and the mixture was stirred at 20° C. for 15 hours. pH of the reaction mixture was adjusted to 6 with hydrochloric acid (1 M in water) and the resulting solution was lyophilized to give a crude product. The crude product was triturated with ethanol to give (2E)-4-(3-fluoroazetidin-1-yl) but-2-enoic acid (300 mg, 0.942 mmol, 32% yield) as a yellow solid.

LC-MS (Method 16): Rₜ=0.100 min; MS (ESIpos): m/z=160.1 [M+H]⁺

### Intermediate 245

### (3-methyl-1H-pyrrolo[2,3-b]pyridin-4-yl) boronic acid

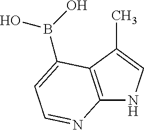

To a solution of 4-bromo-3-methyl-1H-pyrrolo[2,3-b]pyridine (19.0 g, 90.0 mmol) and 4,4,4′,4′,5,5,5′,5′-octamethyl-2,2′-bi-1,3,2-dioxaborolane (34.3 g, 135 mmol) in 1,4-dioxane (500 ml) were added (1,1′-bis(diphenylphosphino) ferrocene) palladium (II) dichloride (6.59 g, 9.00 mmol) and potassium acetate (13.3 g, 135 mmol) at 20° C. The mixture was stirred at 90° C. for 16 hours under nitrogen atmosphere. The reaction mixture was filtered through a pad of celite and the filtrate was concentrated in vacuo to give a residue. The residue was purified by prep-MPLC ([Instrument: GX-A; Column: Waters Xbridge 550*50 mm*5 μm; eluent A: water (0.2% FA), eluent B: acetonitrile; gradient: 0-30 min 5-30% B; flow 100 ml/min; temperature: RT; Detector: UV 220/254 nm]) to give (3-methyl-1H-pyrrolo[2,3-b]pyridin-4-yl) boronic acid (1.50 g, 8.52 mmol, 9% yield) as a brown solid.

LC-MS (Method 20): R₁=0.891 min; MS (ESIpos): m/z=176.8 [M+H]⁺.

### Intermediate 246

### tert-butyl 2-bromo-3-(3-methyl-1H-pyrrolo[2,3-b]pyridin-4-yl)-6,7-dihydropyrazolo[1,5-a]pyrazine-5(4H)-carboxylate

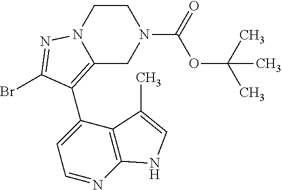 [SMILES: Cc1c[nH]c2nccc(-c3c(Br)nn4c3CN(C(=O)OC(C)(C)C)CC4)c12]

To a solution of tert-butyl 2-bromo-3-iodo-6,7-dihydropyrazolo[1,5-a]pyrazine-5(4H)-carboxylate (400 mg, 0.934 mmol, Intermediate 67) and 3-methyl-1H-pyrrolo[2,3-b]pyridin-4-yl) boronic acid (247 mg, 1.40 mmol, Intermediate 2) in 1,4-dioxane (8.0 ml) and water (2.0 ml) were added sodium carbonate (198 mg, 1.87 mmol) and (1,1′-bis(diphenylphosphino) ferrocene) palladium (II) dichloride (68.4 mg, 0.0934 mmol) at 80° C. The mixture was stirred at 80° C. for 16 hours under nitrogen atmosphere. The reaction mixture was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-bromo-3-(3-methyl-1H-pyrrolo[2,3-b]pyridin-4-yl)-6,7-dihydropyrazolo[1,5-a]pyrazine-5(4H)-carboxylate (90.0 mg, 0.208 mmol, 22% yield) as a yellow oil.

LC-MS (Method 16): Rₜ=0.508 min; MS (ESIpos): m/z=434.0 [M+H]⁺.

### Intermediate 247

### tert-butyl 2-(4-fluorophenyl)-3-(3-methyl-1H-pyrrolo[2,3-b]pyridin-4-yl)-6,7-dihydropyrazolo[1,5-a]pyrazine-5(4H)-carboxylate

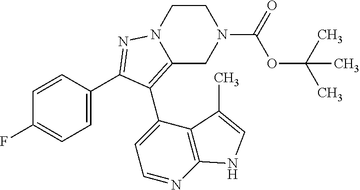 [SMILES: Cc1c[nH]c2nccc(-c3c(-c4ccc(F)cc4)nn4c3CN(C(=O)OC(C)(C)C)CC4)c12]

To a solution of tert-butyl 2-bromo-3-(3-methyl-1H-pyrrolo[2,3-b]pyridin-4-yl)-6,7-dihydropyrazolo[1,5-a]pyrazine-5(4H)-carboxylate (90.0 mg, 0.208 mmol, Intermediate 246) and (4-fluorophenyl) boronic acid (43.7 mg, 0.312 mmol) in 1,4-dioxane (4 ml) and water (1 ml) were added sodium carbonate (44.1 mg, 0.416 mmol) and (1,1′-bis(diphenylphosphino) ferrocene) palladium (II) dichloride (15.2 mg, 0.0208 mmol) at 20° C. The mixture was stirred at 90° C. for 16 hours under nitrogen atmosphere. The reaction mixture was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(4-fluorophenyl)-3-(3-methyl-1H-pyrrolo[2,3-b]pyridin-4-yl)-6,7-dihydropyrazolo[1,5-a]pyrazine-5(4H)-carboxylate (80.0 mg, 0.178 mmol, 86% yield) as a yellow oil.

LC-MS (Method 16): Rₜ=0.877 min; MS (ESIpos): m/z=448.2 [M+H]⁺.

### Intermediate 248

### 2-(4-fluorophenyl)-3-(3-methyl-1H-pyrrolo[2,3-b]pyridin-4-yl)-4,5,6,7-tetrahydropyrazolo[1,5-a]pyrazine hydrochloride (1:1)

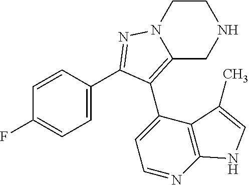 [SMILES: Cc1c[nH]c2nccc(-c3c(-c4ccc(F)cc4)nn4c3CNCC4)c12]

To a solution of tert-butyl 2-(4-fluorophenyl)-3-(3-methyl-1H-pyrrolo[2,3-b]pyridin-4-yl)-6,7-dihydropyrazolo[1,5-a]pyrazine-5(4H)-carboxylate (80.0 mg, 0.179 mmol, Intermediate 247) in ethyl acetate (5.0 ml) was added hydrochloric acid (4M in ethyl acetate, 6.0 ml) at 20° C. The mixture was stirred at 20° C. for 2 hours. The reaction mixture was filtered and the solid cake was collected to give 2-(4-fluorophenyl)-3-(3-methyl-1H-pyrrolo[2,3-b]pyridin-4-yl)-4,5,6,7-tetrahydropyrazolo[1,5-a]pyrazine hydrochloride (1:1) (45.0 mg, 0.117 mmol, 65% yield) as a brown solid.

LC-MS (Method 16): Rₜ=0.469 min; MS (ESIpos): m/z=348.2 [M+H]⁺.

### Intermediate 249

### tert-butyl 2-(2-fluoro-4-(trifluoromethyl)phenyl)-3-(3-methyl-1-((2-(trimethylsilyl) ethoxy)methyl)-1H-pyrrolo[2,3-b]pyridin-4-yl)-6,7-dihydropyrazolo[1,5-a]pyrazine-5(4H)-carboxylate

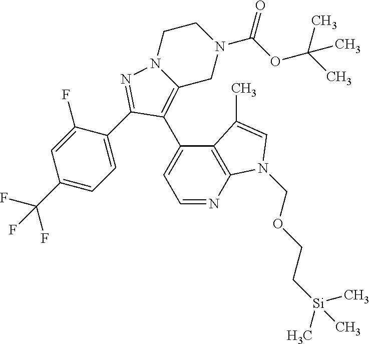 [SMILES: Cc1cn(COCC[Si](C)(C)C)c2nccc(-c3c(-c4ccc(C(F)(F)F)cc4F)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (300 mg, 0.533 mmol, Intermediate 255) and [2-fluoro-4-(trifluoromethyl)phenyl]boronic acid (222 mg, 1.07 mmol) in 1,4-dioxane (12 ml) and water (3.0 ml) were added sodium carbonate (113 mg, 1.07 mmol) and (1,1′-bis(diphenylphosphino) ferrocene) palladium (II) dichloride (78.0 mg, 0.107 mmol) at 20° C. The reaction mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(2-fluoro-4-(trifluoromethyl)phenyl)-3-(3-methyl-1-((2-(trimethylsilyl) ethoxy)methyl)-1H-pyrrolo[2,3-b]pyridin-4-yl)-6,7-dihydropyrazolo[1,5-a]pyrazine-5(4H)-carboxylate (200 mg, 0.310 mmol, 58% yield) as a yellow oil.

LC-MS (Method 16): Rₜ=0.762 min; MS (ESIpos): m/z=646.3 [M+H]⁺.

### Intermediate 250

### (4-(2-(2-fluoro-4-(trifluoromethyl)phenyl)-4,5,6,7-tetrahydropyrazolo[1,5-a]pyrazin-3-yl)-3-methyl-1H-pyrrolo[2,3-b]pyridin-1-yl) methanol

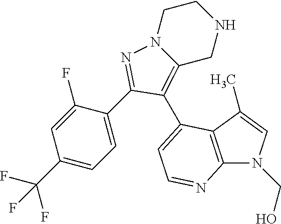 [SMILES: Cc1cn(CO)c2nccc(-c3c(-c4ccc(C(F)(F)F)cc4F)nn4c3CNCC4)c12]

To a solution of tert-butyl 2-[2-fluoro-4-(trifluoromethyl)phenyl]-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (200 mg, 0.310 mmol, Intermediate 249) in dichloromethane (9.0 ml) was added trifluoroacetic acid (4.5 ml) at 20° C. The mixture was stirred at 20° C. for 2 hours. The reaction mixture was concentrated in vacuo to give (4-(2-(2-fluoro-4-(trifluoromethyl)phenyl)-4,5,6,7-tetrahydropyrazolo[1,5-a]pyrazin-3-yl)-3-methyl-1H-pyrrolo[2,3-b]pyridin-1-yl) methanol (130 mg, 0.292 mmol, 94% yield) as a yellow oil.

LC-MS (Method 16): Rₜ=0.456 min; MS (ESIpos): m/z=446.2 [M+H]⁺.

### Intermediate 251

### 2-(2-fluoro-4-(trifluoromethyl)phenyl)-3-(3-methyl-1H-pyrrolo[2,3-b]pyridin-4-yl)-4,5,6,7-tetrahydropyrazolo[1,5-a]pyrazine

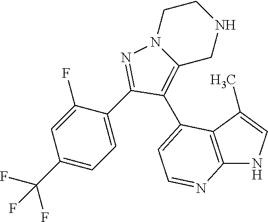 [SMILES: Cc1c[nH]c2nccc(-c3c(-c4ccc(C(F)(F)F)cc4F)nn4c3CNCC4)c12]

To a solution of (4-{2-[2-fluoro-4-(trifluoromethyl)phenyl]-4,5,6,7-tetrahydropyrazolo[1,5-a]pyrazin-3-yl}-3-methyl-1H-pyrrolo[2,3-b]pyridin-1-yl) methanol (130 mg, 0.292 mmol, Intermediate 250) in tetrahydrofuran (8.0 ml) was added sodium hydroxide (2 M in water, 4.0 ml) at 20° C. The mixture was stirred at 20° C. for 4 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 150*40 mm*15 μm; mobile phase: water (TFA)-ACN; B %: 12%-42%, 10 min) to give 2-(2-fluoro-4-(trifluoromethyl)phenyl)-3-(3-methyl-1H-pyrrolo[2,3-b]pyridin-4-yl)-4,5,6,7-tetrahydropyrazolo[1,5-a]pyrazine (50.0 mg, 0.120 mmol, 41% yield) as a white solid.

LC-MS (Method 16): Rₜ=0.486 min; MS (ESIpos): m/z=416.2 [M+H]⁺.

### Intermediate 252

### 3-chloro-1-(2-(2-fluoro-4-(trifluoromethyl)phenyl)-3-(3-methyl-1H-pyrrolo[2,3-b]pyridin-4-yl)-6,7-dihydropyrazolo[1,5-a]pyrazin-5(4H)-yl) propan-1-one

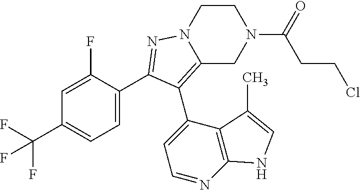 [SMILES: Cc1c[nH]c2nccc(-c3c(-c4ccc(C(F)(F)F)cc4F)nn4c3CN(C(=O)CCCl)CC4)c12]

To a solution of 2-[2-fluoro-4-(trifluoromethyl)phenyl]-3-(3-methyl-1H-pyrrolo[2,3-b]pyridin-4-yl)-4,5,6,7-tetrahydropyrazolo[1,5-a]pyrazine (40.0 mg, 0.0963 mmol, Intermediate 251) and 3-chloropropanoyl chloride (12.2 mg, 0.0963 mmol) in dichloromethane (12 ml) was added trimethylamine (0.027 ml, 0.193 mmol) at −20° C. The mixture was stirred at −20° C. for 1 hour. The reaction mixture was concentrated in vacuo to give 3-chloro-1-(2-(2-fluoro-4-(trifluoromethyl)phenyl)-3-(3-methyl-1H-pyrrolo[2,3-b]pyridin-4-yl)-6,7-dihydropyrazolo[1,5-a]pyrazin-5(4H)-yl) propan-1-one (45.0 mg, 0.0890 mmol, 92% yield) as a yellow solid.

LC-MS (Method 16): Rₜ=0.825 min; MS (ESIpos): m/z=470.2 [(M−36)+H]⁺.

### Intermediate 253

### 4-bromo-3-methyl-1-{[2-(trimethylsilyl) ethoxy]methyl}-1H-pyrrolo[2,3-b]pyridine

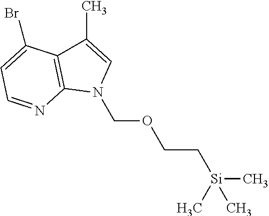 [SMILES: Cc1cn(COCC[Si](C)(C)C)c2nccc(Br)c12]

To a solution of 4-bromo-3-methyl-1H-pyrrolo[2,3-b]pyridine (10.0 g, 47.4 mmol) in tetrahydrofuran (250 ml) was added sodium hydride (2.27 g, 56.9 mmol) at 0° C. After stirring at 0° C. for 0.5 hour, to the mixture above was added [2-(chloromethoxy)ethyl](trimethyl) silane (9.48 g, 56.9 mmol) and the resulting mixture was stirred at 20° C. for 16 hours. The reaction was quenched with water and the resulting mixture was extracted with ethyl acetate. The combined organic phase was concentrated in vacuo to give a residue. The residue was purified by column chromatography on silica gel (petroleum ether:ethyl acetate=100:1 to 10:1) to give 4-bromo-3-methyl-1-{[2-(trimethylsilyl) ethoxy]methyl}-1H-pyrrolo[2,3-b]pyridine (9.25 g, 27.1 mmol, 57% yield) as a colorless oil.

LC-MS (Method 16): R₁=1.141 min; MS (ESIpos): m/z=343.1 [M+H]⁺.

### Intermediate 254

### 3-methyl-4-(4,4,5,5-tetramethyl-1,3,2-dioxaborolan-2-yl)-1-{[2-(trimethylsilyl) ethoxy]methyl}-1H-pyrrolo[2,3-b]pyridine

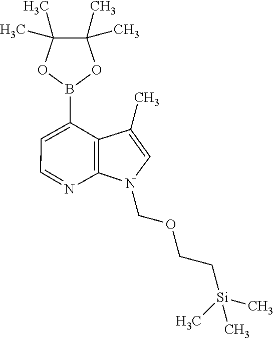

To a solution of 4-bromo-3-methyl-1-{[2-(trimethylsilyl) ethoxy]methyl}-1H-pyrrolo[2,3-b]pyridine (9.20 g, 27.0 mmol, Intermediate 253) and 4,4,4′,4′,5,5,5′,5′-octamethyl-2,2′-bi-1,3,2-dioxaborolane (10.3 g, 40.4 mmol) in 1,4-dioxane (220 ml) were added potassium acetate (3.97 g, 40.4 mmol) and (1,1′-bis(diphenylphosphino) ferrocene) palladium (II) dichloride (1.97 g, 2.70 mmol) at 20° C. The mixture was stirred at 90° C. for 16 hours under nitrogen atmosphere. The reaction mixture was poured into water and extracted with ethyl acetate. The combined organic phase was concentrated in vacuo to give a residue. The residue was purified by column chromatography on silica gel (petroleum ether:ethyl acetate=100:1 to 10:1) to give 3-methyl-4-(4,4,5,5-tetramethyl-1,3,2-dioxaborolan-2-yl)-1-{[2-(trimethylsilyl) ethoxy]methyl)-1H-pyrrolo[2,3-b]pyridine (10.0 g, 25.7 mmol, 96% yield) as a light green oil.

LC-MS (Method 16): Rₜ=1.137 min; MS (ESIpos): m/z=389.3 [M+H]⁺.

### Intermediate 255

### tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

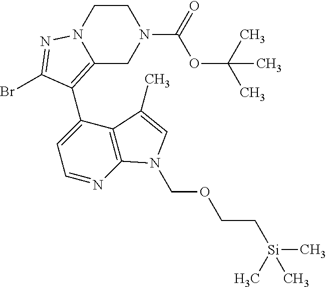 [SMILES: Cc1cn(COCC[Si](C)(C)C)c2nccc(-c3c(Br)nn4c3CN(C(=O)OC(C)(C)C)CC4)c12]

To a solution of 2-bromo-3-iodo-6,7-dihydropyrazolo[1,5-a]pyrazine-5(4H)-carboxylate (1.50 g, 3.50 mmol, Intermediate 67) and 3-methyl-4-(4,4,5,5-tetramethyl-1,3,2-dioxaborolan-2-yl)-1-{[2-(trimethylsilyl) ethoxy]methyl}-1H-pyrrolo[2,3-b]pyridine (1.50 g, 3.85 mmol, Intermediate 254) in 1,4-dioxane (60 ml) and water (15 ml) were added sodium carbonate (0.743 g, 7.00 mmol) and (1,1′-bis(diphenylphosphino) ferrocene) palladium (II) dichloride (0.256 g, 0.350 mmol) at 20° C. The mixture was stirred at 60° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (1.20 g, 2.13 mmol, 61% yield) as a yellow oil.

LC-MS (Method 16): Rₜ=1.062 min; MS (ESIpos): m/z=564.6 [M+H]⁺.

### Intermediate 256

### tert-butyl 2-(4-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

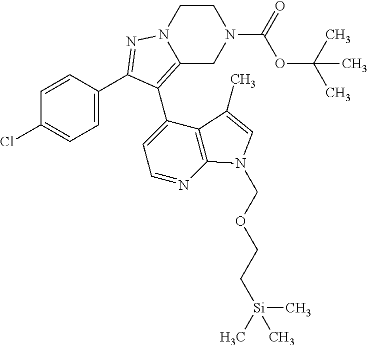 [SMILES: Cc1cn(COCC[Si](C)(C)C)c2nccc(-c3c(-c4ccc(Cl)cc4)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (400 mg, 0.711 mmol, Intermediate 255) and (4-chlorophenyl) boronic acid (222 mg, 1.42 mmol) in 1,4-dioxane (8.0 ml) and water (2.0 ml) were added sodium carbonate (151 mg, 1.42 mmol) and (1,1′-bis(diphenylphosphino) ferrocene) palladium (II) dichloride (104 mg, 0.142 mmol) at 90° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(4-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (350 mg, 0.589 mmol, 83% yield) as a yellow oil.

LC-MS (Method 16): Rₜ=0.732 min; MS (ESIpos): m/z=594.2 [M+H]⁺.

### Intermediate 257

### 2-(4-chlorophenyl)-3-(3-methyl-1H-pyrrolo[2,3-b]pyridin-4-yl)-4,5,6,7-tetrahydropyrazolo[1,5-a]pyrazine trifluoroacetate (1:1)

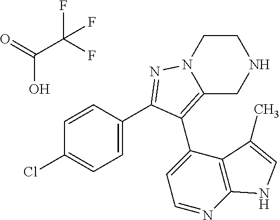

The solution of tert-butyl 2-(4-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (350 mg, 0.589 mmol, Intermediate 256) in dichloromethane (12 ml) was added trifluoroacetic acid (6 ml) at 20° C. The mixture was stirred at 20° C. for 12 hours. The reaction solution was poured into water and extracted with ethyl acetate. The combined organic layers were washed with brine, dried over anhydrous sodium sulfate, filtered and the filtrate was concentrated in vacuo to give 2-(4-chlorophenyl)-3-(3-methyl-1H-pyrrolo[2,3-b]pyridin-4-yl)-4,5,6,7-tetrahydropyrazolo[1,5-a]pyrazine trifluoroacetate (1/1) (200 mg, 0.419 mmol, 71% yield) as a yellow solid.

LC-MS (Method 16): Rₜ=0.478 min; MS (ESIpos): m/z=364.1 [M+H]⁺.

### Intermediate 258

### tert-butyl 2-(3-chloro-2-fluorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

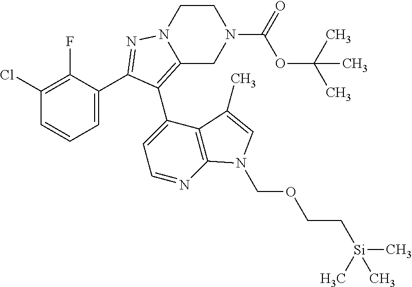 [SMILES: Cc1cn(COCC[Si](C)(C)C)c2nccc(-c3c(-c4cccc(Cl)c4F)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (300 mg, 0.533 mmol, Intermediate 255) and (3-chloro-2-fluorophenyl) boronic acid (372 mg, 2.13 mmol) in a mixed solvent of 1,4-dioxane (12 ml) and water (3 ml) were added sodium carbonate (147 mg, 1.067 mmol) and 1,1-bis(diphenylphosphino) ferrocene-palladium (II) dichloride (39.0 mg, 0.0533 mmol) at 20° C. The mixture was stirred at 100° C. for 16 hours under nitrogen atmosphere. The reaction mixture was concentrated under reduced pressure to give a residue. The residue was purified by silica gel column chromatography (petroleum ether:ethyl acetate=5:1 to 1:1) to give tert-butyl 2-(3-chloro-2-fluorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (230 mg, 0.376 mmol, 70% yield) as a yellow oil.

LC-MS (Method 16): Rₜ=0.734 min; MS (ESIpos): m/z=612.3 [M+H]⁺.

### Intermediate 259

### {4-[2-(3-chloro-2-fluorophenyl)-4,5,6,7-tetrahydropyrazolo[1,5-a]pyrazin-3-yl]-3-methyl-1H-pyrrolo[2,3-b]pyridin-1-yl}methanol trifluoroacetate (1/1)

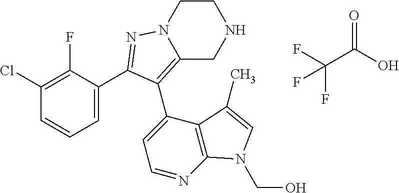 [SMILES: Cc1cn(CO)c2nccc(-c3c(-c4cccc(Cl)c4F)nn4c3CNCC4)c12 ; O=C(O)C(F)(F)F]

To a solution of tert-butyl 2-(3-chloro-2-fluorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (230 mg, 0.376 mmol, Intermediate 258) in dichloromethane (8 ml) was added trifluoroacetic acid (4 ml, 51.9 mmol) at 25° C. The mixture was stirred at 25° C. for 4 hours. The reaction mixture was concentrated under reduced pressure to give {4-[2-(3-chloro-2-fluorophenyl)-4,5,6,7-tetrahydropyrazolo[1,5-a]pyrazin-3-yl]-3-methyl-1H-pyrrolo[2,3-b]pyridin-1-yl}methanol (300 mg, 0.728 mmol, 194% yield) as a yellow oil.

LC-MS (Method 16): Rₜ=0.427 min; MS (ESIpos): m/z=412.1 [M+H]⁺.

### Intermediate 260

### 2-(3-chloro-2-fluorophenyl)-3-(3-methyl-1H-pyrrolo[2,3-b]pyridin-4-yl)-4,5,6,7-tetrahydropyrazolo[1,5-a]pyrazine

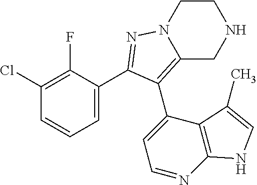 [SMILES: Cc1c[nH]c2nccc(-c3c(-c4cccc(Cl)c4F)nn4c3CNCC4)c12]

To a solution of {4-[2-(3-chloro-2-fluorophenyl)-4,5,6,7-tetrahydropyrazolo[1,5-a]pyrazin-3-yl]-3-methyl-1H-pyrrolo[2,3-b]pyridin-1-yl}methanol (300 mg, 0.728 mmol, Intermediate 259) in tetrahydrofuran (5 ml) was added sodium hydroxide (2 M in water, 5 ml) at 20° C. The mixture was stirred at 20° C. for 4 hours. The reaction mixture was poured into water and extracted with ethyl acetate. The combined organic layers were washed with brine, dried over anhydrous sodium sulfate, filtered and the filtrate was concentrated under reduced pressure to give a residue. The residue was purified by preparative HPLC [Instrument: Gilson-281; Column: Phenomenex luna C18 150*25 mm*10 μm; eluent A: water (0.225% formic acid), eluent B: acetonitrile; gradient: 0-10 min 8-38% B; flow 25 ml/min; temperature: RT; Detector: UV 220/254 nm] to give 2-(3-chloro-2-fluorophenyl)-3-(3-methyl-1H-pyrrolo[2,3-b]pyridin-4-yl)-4,5,6,7-tetrahydropyrazolo[1,5-a]pyrazine (90 mg, 0.235 mmol, 32% yield) as a white solid.

LC-MS (Method 16): Rₜ=0.417 min; MS (ESIpos): m/z=382.1 [M+H]⁺.

### Intermediate 261

### 3-chloro-1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl]propan-1-one

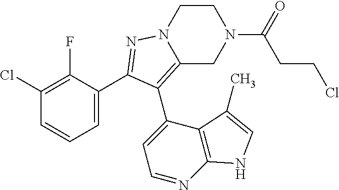 [SMILES: Cc1c[nH]c2nccc(-c3c(-c4cccc(Cl)c4F)nn4c3CN(C(=O)CCCl)CC4)c12]

To a solution of 2-(3-chloro-2-fluorophenyl)-3-(3-methyl-1H-pyrrolo[2,3-b]pyridin-4-yl)-4,5,6,7-tetrahydropyrazolo[1,5-a]pyrazine (85.0 mmol, 0.223 mmol, Intermediate 260) and trimethylamine (45.1 mg, 0.445 mmol) in dichloromethane (15 ml) was added 3-chloropropanoyl chloride (31.1 mg, 0.245 mmol) at −10° C. The mixture was stirred at 25° C. for 12 hours. The reaction mixture was concentrated under reduced pressure to give 3-chloro-1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl]propan-1-one (150 mg, 0.318 mmol, 143% yield) as a yellow solid.

LC-MS (Method 16): Rₜ=0.714 min; MS (ESIpos): m/z=472.0 [M+H]⁺.

### Intermediate 262

### tert-butyl 2-(3-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

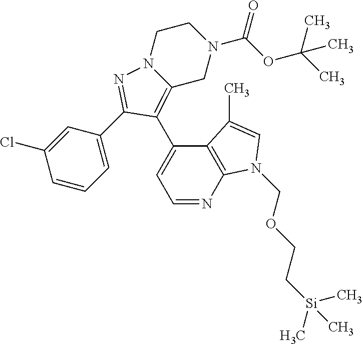 [SMILES: Cc1cn(COCC[Si](C)(C)C)c2nccc(-c3c(-c4cccc(Cl)c4)nn4c3CN(C(=O)OC(C)(C)C)CC4)c12]

To a solution of tert-butyl 2-bromo-3-(3-methyl-1-{[2-(trimethylsily) ethoxy]methyl}-1H-pyrrolo[2,3-b]pyridin-4-yl)-6,7-dihydropyrazolo[1,5-a]pyrazine-5(4H)-carboxylate (400 mg, 0.711 mmol, Intermediate 255) and (3-chlorophenyl) boronic acid (222 mg, 1.42 mmol) in 1,4-dioxane (8.0 ml) and water (2.0 ml) were added sodium carbonate (151 mg, 1.42 mmol) and (1,1′-bis(diphenylphosphino) ferrocene) palladium (II) dichloride (52.0 mg, 0.0711 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(3-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (370 mg, 0.623 mmol, 88% yield) as a yellow oil.

LC-MS (Method 16): Rₜ=0.785 min; MS (ESIpos): m/z=594.4 [M+H]⁺.

### Intermediate 263

### 2-(3-chlorophenyl)-3-(3-methyl-1H-pyrrolo[2,3-b]pyridin-4-yl)-4,5,6,7-tetrahydropyrazolo[1,5-a]pyrazine trifluoroacetate (1/1)

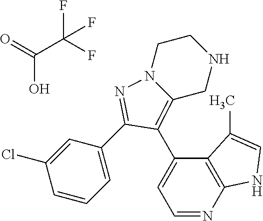

The solution of tert-butyl 2-(3-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (320 mg, 0.539 mmol, Intermediate 262) in dichloromethane (3.0 ml) was added trifluoroacetic acid (1.0 ml) at 20° C. The mixture was stirred at 20° C. for 12 hours. The reaction solution was poured into water and extracted with ethyl acetate. The combined organic layers were washed with brine, dried over anhydrous sodium sulfate, filtered and the filtrate was concentrated in vacuo to give 2-(3-chlorophenyl)-3-(3-methyl-1H-pyrrolo[2,3-b]pyridin-4-yl)-4,5,6,7-tetrahydropyrazolo[1,5-a]pyrazine trifluoroacetate (1/1) (180 mg, 0.377 mmol, 70% yield) as a white solid.

LC-MS (Method 16): Rₜ=0.719 min; MS (ESIpos): m/z=364.1 [M+H]⁺.

### Intermediate 264

### tert-butyl 3-(3-methyl-1-((2-(trimethylsilyl) ethoxy)methyl)-1H-pyrrolo[2,3-b]pyridin-4-yl)-2-(3-(trifluoromethyl)phenyl)-6,7-dihydropyrazolo[1,5-a]pyrazine-5(4H)-carboxylate

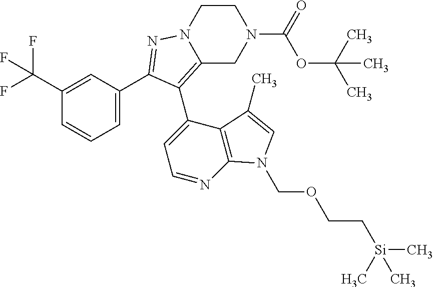 [SMILES: Cc1cn(COCC[Si](C)(C)C)c2nccc(-c3c(-c4cccc(C(F)(F)F)c4)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (400 mg, 0.711 mmol, Intermediate 255) and [3-(trifluoromethyl)phenyl]boronic acid (270 mg, 1.42 mmol) in 1,4-dioxane (8.0 ml) and water (2.0 ml) were added sodium carbonate (151 mg, 1.42 mmol) and (1,1′-bis(diphenylphosphino) ferrocene) palladium (II) dichloride (52.0 mg, 0.0711 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 3-(3-methyl-1-((2-(trimethylsilyl) ethoxy)methyl)-1H-pyrrolo[2,3-b]pyridin-4-yl)-2-(3-(trifluoromethyl)phenyl)-6,7-dihydropyrazolo[1,5-a]pyrazine-5(4H)-carboxylate (300 mg, 0.478 mmol, 67% yield) as a yellow oil.

LC-MS (Method 16): R₁=0.767 min; MS (ESIpos): m/z=628.4 [M+H]⁺.

### Intermediate 265

### (3-methyl-4-{2-[3-(trifluoromethyl)phenyl]-4,5,6,7-tetrahydropyrazolo[1,5-a]pyrazin-3-yl}-1H-pyrrolo[2,3-b]pyridin-1-yl) methanol

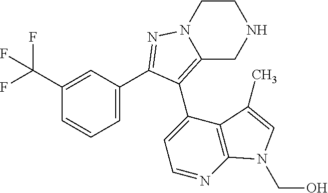 [SMILES: Cc1cn(CO)c2nccc(-c3c(-c4cccc(C(F)(F)F)c4)nn4c3CNCC4)c12]

To a solution of tert-butyl 3-(3-methyl-1-((2-(trimethylsilyl) ethoxy)methyl)-1H-pyrrolo[2,3-b]pyridin-4-yl)-2-(3-(trifluoromethyl)phenyl)-6,7-dihydropyrazolo[1,5-a]pyrazine-5(4H)-carboxylate (300 mg, 0.478 mmol, Intermediate 264) in dichloromethane (8.0 ml) was added trifluoroacetic acid (4.0 ml, 51.9 mmol) at 20° C. The mixture was stirred at 20° C. for 2 hours. The reaction mixture was concentrated in vacuo to give (3-methyl-4-{2-[3-(trifluoromethyl)phenyl]-4,5,6,7-tetrahydropyrazolo[1,5-a]pyrazin-3-yl}-1H-pyrrolo[2,3-b]pyridin-1-yl) methanol (200 mg, 0.468 mmol, 98% yield) as a yellow oil.

LC-MS (Method 16): Rₜ=0.677 min; MS (ESIpos): m/z=428.1 [M+H]⁺.

### Intermediate 266

### 3-(3-methyl-1H-pyrrolo[2,3-b]pyridin-4-yl)-2-(3-(trifluoromethyl)phenyl)-4,5,6,7-tetrahydropyrazolo[1,5-a]pyrazine

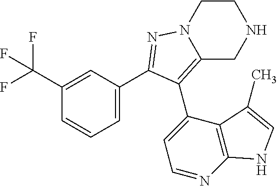 [SMILES: Cc1c[nH]c2nccc(-c3c(-c4cccc(C(F)(F)F)c4)nn4c3CNCC4)c12]

To a solution of (3-methyl-4-{2-[3-(trifluoromethyl)phenyl]-4,5,6,7-tetrahydropyrazolo[1,5-a]pyrazin-3-yl}-1H-pyrrolo[2,3-b]pyridin-1-yl) methanol (200 mg, 0.467 mmol, Intermediate 265) in tetrahydrofuran (6.4 ml) was added sodium hydroxide (2 M in water, 3.2 ml) at 20° C. The mixture was stirred at 20° C. for 4 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 150*40 mm*15 μm; mobile phase: water (TFA)-ACN; B %: 10%-40%, 15 min) to give 3-(3-methyl-1H-pyrrolo[2,3-b]pyridin-4-yl)-2-(3-(trifluoromethyl)phenyl)-4,5,6,7-tetrahydropyrazolo[1,5-a]pyrazine (60.0 mg, 0.151 mmol, 32% yield) as a white solid.

LC-MS (Method 16): Rₜ=0.509 min; MS (ESIpos): m/z=398.2 [M+H]⁺.

### Intermediate 267

### 3-chloro-1-[3-(3-methyl-1H-pyrrolo[2,3-b]pyridin-4-yl)-2-[3-(trifluoromethyl)phenyl]-6,7-dihydropyrazolo[1,5-a]pyrazin-5(4H)-yl]propan-1-one

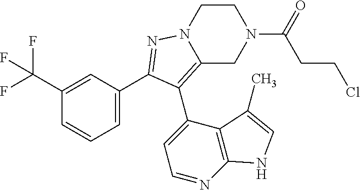 [SMILES: Cc1c[nH]c2nccc(-c3c(-c4cccc(C(F)(F)F)c4)nn4c3CN(C(=O)CCCl)CC4)c12]

To a solution of 3-(3-methyl-1H-pyrrolo[2,3-b]pyridin-4-yl)-2-(3-(trifluoromethyl)phenyl)-4,5,6,7-tetrahydropyrazolo[1,5-a]pyrazine (50.0 mg, 0.126 mmol, Intermediate 266) and 3-chloropropanoyl chloride (19.2 mg, 0.151 mmol) in dichloromethane (5.0 ml) was added trimethylamine (0.035 ml, 0.252 mmol) at −20° C. The mixture was stirred at −20° C. for 1 hour. The reaction mixture was concentrated in vacuo to give 3-chloro-1-[3-(3-methyl-1H-pyrrolo[2,3-b]pyridin-4-yl)-2-[3-(trifluoromethyl)phenyl]-6,7-dihydropyrazolo[1,5-a]pyrazin-5(4H)-yl]propan-1-one (60.0 mg, 0.123 mmol, 98% yield) as a yellow solid.

LC-MS (Method 16): Rₜ=0.837 min; MS (ESIpos): m/z=452.2 [(M−36)+H]⁺.

### Intermediate 268

### tert-butyl 2-(2-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

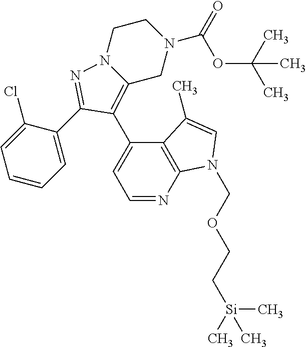 [SMILES: Cc1cn(COCC[Si](C)(C)C)c2nccc(-c3c(-c4ccccc4Cl)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (400 mg, 0.711 mmol, Intermediate 255) and (2-chlorophenyl) boronic acid (222 mg, 1.42 mmol) in 1,4-dioxane (8.0 ml) and water (2.0 ml) were added sodium carbonate (151 mg, 1.42 mmol) and (1,1′-bis(diphenylphosphino) ferrocene) palladium (II) dichloride (104 mg, 0.142 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(2-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (390 mg, 0.656 mmol, 92% yield) as a yellow oil.

LC-MS (Method 16): Rₜ=0.745 min; MS (ESIpos): m/z=594.2 [M+H]⁺.

### Intermediate 269

### 2-(2-chlorophenyl)-3-(3-methyl-1-{[2-(trimethylsilyl) ethoxy]methyl}-1H-pyrrolo[2,3-b]pyridin-4-yl)-4,5,6,7-tetrahydropyrazolo[1,5-a]pyrazine hydrogen chloride (1/1)

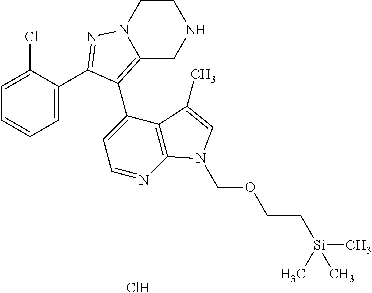 [SMILES: Cc1cn(COCC[Si](C)(C)C)c2nccc(-c3c(-c4ccccc4Cl)nn4c3CNCC4)c12 ; Cl]

To a solution of tert-butyl 2-(2-chlorophenyl)-3-(3-methyl-1-{[2-(trimethylsilyl)ethoxy]methyl}-1H-pyrrolo[2,3-b]pyridin-4-yl)-6,7-dihydropyrazolo[1,5-a]pyrazine-5(4H)-carboxylate (240 mg, 0.404 mmol, Intermediate 268) in ethyl acetate (8.0 ml) was added hydrochloric acid (4M in ethyl acetate, 0.5 ml) at 20° C. The mixture was stirred at 20° C. for 2 hours. The reaction mixture was concentrated in vacuo to give a residue. The residue was purified by trituration with ethyl acetate. The resulting suspension was filtered and the solid cake was collected to give 2-(2-chlorophenyl)-3-(3-methyl-1-{[2-(trimethylsilyl) ethoxy]methyl}-1H-pyrrolo[2,3-b]pyridin-4-yl)-4,5,6,7-tetrahydropyrazolo[1,5-a]pyrazine hydrogen chloride (1/1) (180 mg, 0.339 mmol, 84% yield) as a white solid.

LC-MS (Method 16): Rₜ=0.570 min; MS (ESIpos): m/z=494.2 [M+H]⁺.

### Intermediate 270

### 1-[2-(2-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazin-5(4H)-yl]prop-2-en-1-one

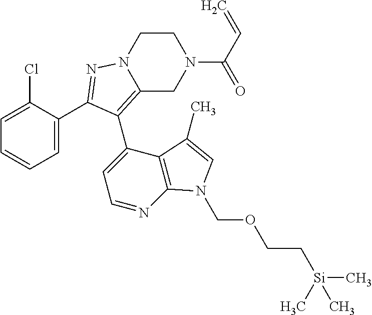 [SMILES: C=CC(=O)N1CCn2nc(-c3ccccc3Cl)c(-c3ccnc4c3c(C)cn4COCC[Si](C)(C)C)c2C1]

To a solution of 2-(2-chlorophenyl)-3-(3-methyl-1-{[2-(trimethylsilyl) ethoxy]methyl}-1H-pyrrolo[2,3-b]pyridin-4-yl)-4,5,6,7-tetrahydropyrazolo[1,5-a]pyrazine hydrogen chloride (1/1) (260 mg, 0.490 mmol, Intermediate 269) and prop-2-enoyl chloride (53.2 mg, 0.588 mmol) in dichloromethane (3.9 ml) was added trimethylamine (0.14 ml, 0.980 mmol) at 0° C. The mixture was stirred at 0° C. for 1 hour. The reaction mixture was concentrated in vacuo to give 1-[2-(2-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazin-5(4H)-yl]prop-2-en-1-one (180 mg, 0.328 mmol, 67% yield) as a yellow solid.

LC-MS (Method 16): Rₜ=0.985 min; MS (ESIpos): m/z=548.2 [M+H]⁺.

### Intermediate 271

### tert-butyl 2-(5-chloro-2-fluorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

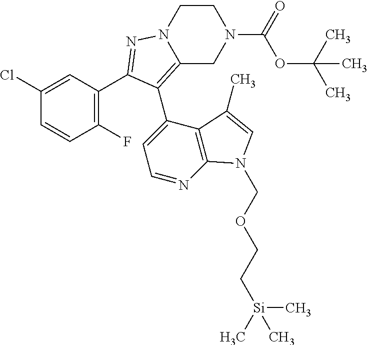 [SMILES: Cc1cn(COCC[Si](C)(C)C)c2nccc(-c3c(-c4cc(Cl)ccc4F)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (400 mg, 0.711 mmol, Intermediate 255) and (5-chloro-2-fluorophenyl) boronic acid (248 mg, 1.42 mmol) in 1,4-dioxane (16 ml) and water (4.0 ml) were added sodium carbonate (151 mg, 1.42 mmol) and (1,1′-bis(diphenylphosphino) ferrocene) palladium (II) dichloride (104 mg, 0.142 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(5-chloro-2-fluorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (350 mg, 0.572 mmol, 80% yield) as a yellow oil.

LC-MS (Method 16): Rₜ=0.749 min; MS (ESIpos): m/z=612.3 [M+H]⁺.

### Intermediate 272

### {4-[2-(5-chloro-2-fluorophenyl)-4,5,6,7-tetrahydropyrazolo[1,5-a]pyrazin-3-yl]-3-methyl-1H-pyrrolo[2,3-b]pyridin-1-yl}methanol

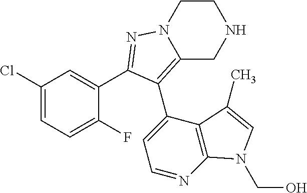 [SMILES: Cc1cn(CO)c2nccc(-c3c(-c4cc(Cl)ccc4F)nn4c3CNCC4)c12]

To a solution of 2-(5-chloro-2-fluorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (350 mg, 0.572 mmol, Intermediate 271) in dichloromethane (6.0 ml) was added trifluoroacetic acid (3.0 ml) at 20° C. The mixture was stirred at 20° C. for 2 hours. The reaction mixture was concentrated in vacuo to give {4-[2-(5-chloro-2-fluorophenyl)-4,5,6,7-tetrahydropyrazolo[1,5-a]pyrazin-3-yl]-3-methyl-1H-pyrrolo[2,3-b]pyridin-1-yl}methanol (220 mg, 0.534 mmol, 93% yield) as a brown oil.

LC-MS (Method 16): Rₜ=0.480 min; MS (ESIpos): m/z=412.2 [M+H]⁺.

### Intermediate 273

### 2-(5-chloro-2-fluorophenyl)-3-(3-methyl-1H-pyrrolo[2,3-b]pyridin-4-yl)-4,5,6,7-tetrahydropyrazolo[1,5-a]pyrazine

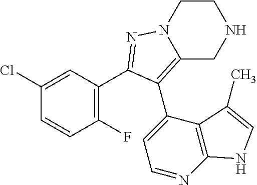 [SMILES: Cc1c[nH]c2nccc(-c3c(-c4cc(Cl)ccc4F)nn4c3CNCC4)c12]

To a solution of {4-[2-(5-chloro-2-fluorophenyl)-4,5,6,7-tetrahydropyrazolo[1,5-a]pyrazin-3-yl]-3-methyl-1H-pyrrolo[2,3-b]pyridin-1-yl}methanol (220 mg, 0.534 mmol, Intermediate 272) in tetrahydrofuran (8.0 ml) was added sodium hydroxide (2 M in water, 4.0 ml) at 20° C. The mixture was stirred at 20° C. for 4 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Phenomenex luna C18 150*25 mm*10 μm; mobile phase: water (TFA)-ACN; B %: 8%-28%, 10 min) to give 2-(5-chloro-2-fluorophenyl)-3-(3-methyl-1H-pyrrolo[2,3-b]pyridin-4-yl)-4,5,6,7-tetrahydropyrazolo[1,5-a]pyrazine (100 mg, 0.262 mmol, 49% yield) as a white solid.

LC-MS (Method 16): Rₜ=0.607 min; MS (ESIpos): m/z=382.1 [M+H]⁺.

### Intermediate 274

### 3-chloro-1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]propan-1-one

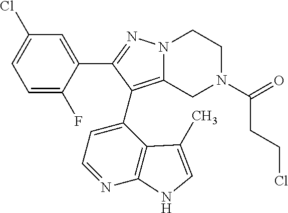 [SMILES: Cc1c[nH]c2nccc(-c3c(-c4cc(Cl)ccc4F)nn4c3CN(C(=O)CCCl)CC4)c12]

To a solution of 2-(5-chloro-2-fluorophenyl)-3-(3-methyl-1H-pyrrolo[2,3-b]pyridin-4-yl)-4,5,6,7-tetrahydropyrazolo[1,5-a]pyrazine (80.0 mg, 0.210 mmol, Intermediate 273) and 3-chloropropanoyl chloride (26.6 mg, 0.210 mmol) in dichloromethane (6.0 ml) was added trimethylamine (0.058 ml, 0.419 mmol) at −20° C. The mixture was stirred at −20° C. for 1 hour. The reaction mixture was concentrated in vacuo to give 3-chloro-1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]propan-1-one (90.0 mg, 0.191 mmol, 91% yield) as a yellow solid.

LC-MS (Method 16): Rₜ=0.482 min; MS (ESIpos): m/z=436.2 [M+H]⁺.

### Intermediate 275

### 6-bromo-2-fluoro-1-benzothiophene

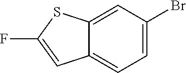 [SMILES: Fc1cc2ccc(Br)cc2s1]

To a solution of 6-bromo-1-benzothiophene (2.00 g, 9.39 mmol) in tetrahydrofuran (25 ml) was added lithium diisopropyl amide (2 M in tetrahydrofuran, 11.7 ml, 23.5 mmol) at −70° C. under nitrogen atmosphere. The mixture was stirred at −70° C. for 1 hour. To the solution above was added dropwise a solution of N-fluoro-N-(phenylsulfonyl)benzenesulfonamide (2.96 g, 9.39 mmol) in tetrahydrofuran (5 ml) at −65° C. and the resulting mixture was stirred at 25° C. for 12 hours. The reaction was quenched with water and the resulting solution was extracted with ethyl acetate. The combined organic layers were washed with brine, dried over sodium sulphate, filtered, the filtrate was concentrated under reduced pressure to give a residue. The residue was purified by silica gel column chromatography (petroleum ether:ethyl acetate=1:0) to give 6-bromo-2-fluoro-1-benzothiophene (730 mg, 3.16 mmol, 34% yield) as a white solid.

¹H NMR (CDC) 3), δ [ppm]=7.80 (s, 1H), 7.54-7.41 (m, 2H), 6.69 (d, J=2.4 Hz, 1H).

### Intermediate 276

### 2-(2-fluoro-1-benzothiophen-6-yl)-4,4,5,5-tetramethyl-1,3,2-dioxaborolane

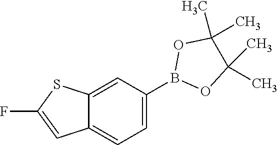 [SMILES: CC1(C)OB(c2ccc3cc(F)sc3c2)OC1(C)C]

To a solution of 6-bromo-2-fluoro-1-benzothiophene (500 mg, 2.16 mmol, Intermediate 275) and 4,4,4′,4′,5,5,5′,5′-octamethyl-2,2′-bi-1,3,2-dioxaborolane (1.65 g, 6.49 mmol) in 1,4-dioxane (10 ml) were added (1,1′-bis(diphenylphosphino) ferrocene) palladium (II) dichloride (158 mg, 0.216 mmol) and potassium acetate (449 mg, 3.25 mmol) at 20° C. The mixture was stirred at 90° C. for 16 hours under nitrogen atmosphere. The reaction mixture was poured into water and extracted with ethyl acetate. The combined organic layers were washed with brine, dried over anhydrous sodium sulphate, filtered and the filtrate was concentrated under reduced pressure to give a residue. The residue was purified by silica gel column chromatography (petroleum ether:ethyl acetate=1:0 to 3:1) to give 2-(2-fluoro-1-benzothiophen-6-yl)-4,4,5,5-tetramethyl-1,3,2-dioxaborolane (1.00 g, 1.80 mmol, 50% purity, 83% yield) as a yellow oil.

¹H NMR (400 MHz, CDCl₃), δ [ppm]=8.14 (s, 1H), 7.76 (d, J=8.0 Hz, 1H), 7.62 (d, J=8.0 Hz, 1H), 6.71 (d, J=2.0 Hz, 1H), 1.37 (s, 12H).

### Intermediate 277

### tert-butyl 2-(2-fluorobenzo[b]thiophen-6-yl)-3-(3-methyl-1-((2-(trimethylsilyl) ethoxy)methyl)-1H-pyrrolo[2,3-b]pyridin-4-yl)-6,7-dihydropyrazolo[1,5-a]pyrazine-5(4H)-carboxylate

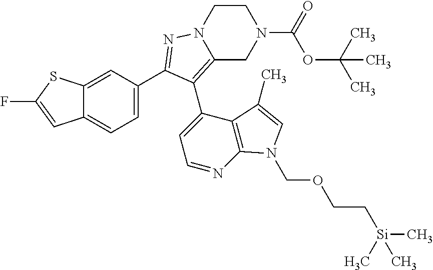 [SMILES: Cc1cn(COCC[Si](C)(C)C)c2nccc(-c3c(-c4ccc5cc(F)sc5c4)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (600 mg, 1.07 mmol, Intermediate 255) and 2-(2-fluoro-1-benzothiophen-6-yl)-4,4,5,5-tetramethyl-1,3,2-dioxaborolane (593 mg, 2.13 mmol, Intermediate 276) in 1,4-dioxane (24 ml) and water (6.0 ml) were added sodium carbonate (227 mg, 2.13 mmol) and (1,1′-bis(diphenylphosphino) ferrocene) palladium (II) dichloride (78.0 mg, 0.107 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(2-fluorobenzo[b]thiophen-6-yl)-3-(3-methyl-1-((2-(trimethylsilyl) ethoxy)methyl)-1H-pyrrolo[2,3-b]pyridin-4-yl)-6,7-dihydropyrazolo[1,5-a]pyrazine-5(4H)-carboxylate (600 mg, 0.947 mmol, 89% yield) as a yellow oil.

LC-MS (Method 16): Rₜ=0.730 min; MS (ESIpos): m/z=634.2 [M+H]⁺

### Intermediate 278

### {4-[2-(2-fluoro-1-benzothiophen-6-yl)-4,5,6,7-tetrahydropyrazolo[1,5-a]pyrazin-3-yl]-3-methyl-1H-pyrrolo[2,3-b]pyridin-1-yl}methanol

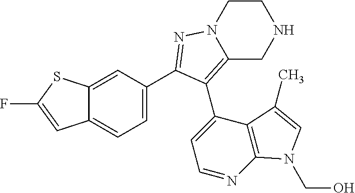 [SMILES: Cc1cn(CO)c2nccc(-c3c(-c4ccc5cc(F)sc5c4)nn4c3CNCC4)c12]

To a solution of tert-butyl 2-(2-fluoro-1-benzothiophen-6-yl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (600 mg, 0.947 mmol, Intermediate 277) in dichloromethane (10 ml) was added trifluoroacetic acid (5.0 ml, 64.9 mmol) at 20° C. The mixture was stirred at 20° C. for 2 hours. The reaction mixture was concentrated in vacuo to give {4-[2-(2-fluoro-1-benzothiophen-6-yl)-4,5,6,7-tetrahydropyrazolo[1,5-a]pyrazin-3-yl]-3-methyl-1H-pyrrolo[2,3-b]pyridin-1-yl}methanol (400 mg, 0.923 mmol, 97% yield) as a yellow oil.

LC-MS (Method 16): Rₜ=0.443 min; MS (ESIpos): m/z=434.1 [M+H]⁺.

### Intermediate 279

### 2-(2-fluoro-1-benzothiophen-6-yl)-3-(3-methyl-1H-pyrrolo[2,3-b]pyridin-4-yl)-4,5,6,7-tetrahydropyrazolo[1,5-a]pyrazine

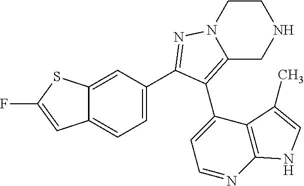 [SMILES: Cc1c[nH]c2nccc(-c3c(-c4ccc5cc(F)sc5c4)nn4c3CNCC4)c12]

To a solution of {4-[2-(2-fluoro-1-benzothiophen-6-yl)-4,5,6,7-tetrahydropyrazolo[1,5-a]pyrazin-3-yl]-3-methyl-1H-pyrrolo[2,3-b]pyridin-1-yl}methanol (400 mg, 0.923 mmol, Intermediate 278) in tetrahydrofuran (10 ml) was added sodium hydroxide (2 M in water, 5.0 ml) at 20° C. The mixture was stirred at 20° C. for 4 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 150*40 mm*15 μm; mobile phase: water (TFA)-ACN; B %: 10%-40%, 15 min) to give 2-(2-fluoro-1-benzothiophen-6-yl)-3-(3-methyl-1H-pyrrolo[2,3-b]pyridin-4-yl)-4,5,6,7-tetrahydropyrazolo[1,5-a]pyrazine (75.0 mg, 0.186 mmol, 20% yield) as a white solid.

LC-MS (Method 16): Rₜ=0.755 min; MS (ESIpos): m/z=404.2 [M+H]⁺.

### Intermediate 280

### 3-chloro-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]propan-1-one

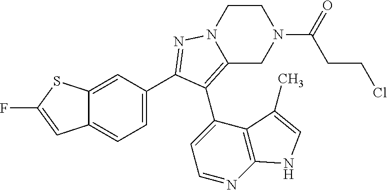 [SMILES: Cc1c[nH]c2nccc(-c3c(-c4ccc5cc(F)sc5c4)nn4c3CN(C(=O)CCCl)CC4)c12]

To a solution of 2-(2-fluoro-1-benzothiophen-6-yl)-3-(3-methyl-1H-pyrrolo[2,3-b]pyridin-4-yl)-4,5,6,7-tetrahydropyrazolo[1,5-a]pyrazine (30.0 mg, 0.0743 mmol, Intermediate 279) in dichloromethane (6.0 ml) were added 3-chloropropanoyl chloride (11.3 mg, 0.0892 mmol) and trimethylamine (0.021 ml, 0.149 mmol) at −20° C. The mixture was stirred at −20° C. for 1 hour. The reaction mixture was concentrated in vacuo to give 3-chloro-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]propan-1-one (30 mg, 0.0607 mmol, 82% yield) as a yellow solid.

LC-MS (Method 16): Rₜ=0.842 min; MS (ESIpos): m/z=458.2 [(M−36)+H]⁺.

### Intermediate 281

### tert-butyl 2-(4-cyanophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

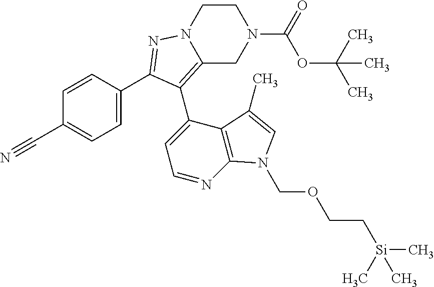 [SMILES: Cc1cn(COCC[Si](C)(C)C)c2nccc(-c3c(-c4ccc(C#N)cc4)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (200 mg, 0.356 mmol, Intermediate 255) and (4-cyanophenyl) boronic acid (104 mg, 0.711 mmol) in 1,4-dioxane (8.0 ml) and water (2.0 ml) were added sodium carbonate (75.4 mg, 0.711 mmol) and (1,1′-bis(diphenylphosphino) ferrocene) palladium (II) dichloride (52.0 mg, 0.0711 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 3:1) to give tert-butyl 2-(4-cyanophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (170 mg, 0.291 mmol, 82% yield) as a yellow oil.

LC-MS (Method 16): Rₜ=0.743 min; MS (ESIpos): m/z=585.3 [M+H]⁺.

### Intermediate 282

### 4-[3-(3-methyl-1H-pyrrolo[2,3-b]pyridin-4-yl)-4,5,6,7-tetrahydropyrazolo[1,5-a]pyrazin-2-yl]benzonitrile

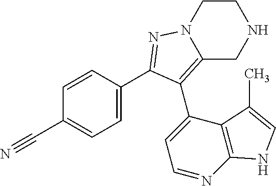 [SMILES: Cc1c[nH]c2nccc(-c3c(-c4ccc(C#N)cc4)nn4c3CNCC4)c12]

To a solution of tert-butyl 2-(4-cyanophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (150 mg, 0.256 mmol, Intermediate 281) in dichloromethane (2.9 ml) was added trifluoroacetic acid (8.8 ml) at 20° C. The mixture was stirred at 20° C. for 2 hours. The reaction mixture was concentrated in vacuo to give a residue. To this residue was added tetrahydrofuran and sodium hydroxide solution (1 M in water, 0.5 ml), and the resulting mixture was stirred at 20° C. for 2 hours. The mixture was poured into water and extracted with ethyl acetate. The combined organic layers were washed with brine, dried over anhydrous sodium sulfate, filtered and the filtrate was concentrated in vacuo to give 4-[3-(3-methyl-1H-pyrrolo[2,3-b]pyridin-4-yl)-4,5,6,7-tetrahydropyrazolo[1,5-a]pyrazin-2-yl]benzonitrile (80.0 mg, 0.226 mmol, 88% yield) as a white solid.

LC-MS (Method 16): Rₜ=0.403 min; MS (ESIpos): m/z=335.2 [M+H]⁺.

### Intermediate 283

### 4-[5-(3-chloropropanoyl)-3-(3-methyl-1H-pyrrolo[2,3-b]pyridin-4-yl)-4,5,6,7-tetrahydropyrazolo[1,5-a]pyrazin-2-yl]benzonitrile

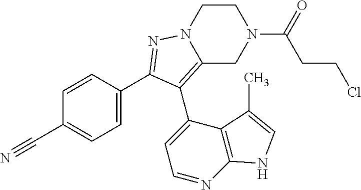 [SMILES: Cc1c[nH]c2nccc(-c3c(-c4ccc(C#N)cc4)nn4c3CN(C(=O)CCCl)CC4)c12]

To a solution of 4-[3-(3-methyl-1H-pyrrolo[2,3-b]pyridin-4-yl)-4,5,6,7-tetrahydropyrazolo[1,5-a]pyrazin-2-yl]benzonitrile (20.0 mg, 0.0564 mmol, Intermediate 282) and 3-chloropropanoyl chloride (8.60 mg, 0.0677 mmol) in dichloromethane (6.0 ml) was added trimethylamine (0.016 ml, 0.113 mmol) at 0° C. The mixture was stirred at 0° C. for 1 hour. The reaction mixture was concentrated in vacuo to give 4-[5-(3-chloropropanoyl)-3-(3-methyl-1H-pyrrolo[2,3-b]pyridin-4-yl)-4,5,6,7-tetrahydropyrazolo[1,5-a]pyrazin-2-yl]benzonitrile (20.0 mg, 0.0400 mmol, 80% yield) as a yellow solid.

LC-MS (Method 16): Rₜ=0.460 min; MS (ESIpos): m/z=409.2 [M+H]⁺.

### Intermediate 284

### tert-butyl 2-(1-benzofuran-7-yl)-3-(3-methyl-1-{[2-(trimethylsilyl) ethoxy]methyl}-1H-pyrrolo [2,3-b]pyridin-4-yl)-6,7-dihydropyrazolo[1,5-a]pyrazine-5(4H)-carboxylate

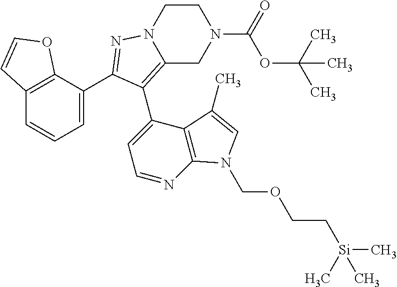 [SMILES: Cc1cn(COCC[Si](C)(C)C)c2nccc(-c3c(-c4cccc5ccoc45)nn4c3CN(C(=O)OC(C)(C)C)CC4)c12]

To a solution of tert-butyl 2-bromo-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (300 mg, 0.533 mmol, Intermediate 255) and 1-benzofuran-7-ylboronic acid (345 mg, 2.13 mmol) in a mixed solvent of 1,4-dioxane (5 ml) and water (1 ml) were added cesium carbonate (434 mg, 1.33 mmol) and 1,1-bis(diphenylphosphino) ferrocene-palladium (II) dichloride (39.0 mg, 0.0533 mmol) at 20° C. The mixture was stirred at 90° C. for 16 hours under nitrogen atmosphere. The reaction mixture was concentrated under reduced pressure to give a residue. The residue was purified by silica gel column chromatography (petroleum ether:ethyl acetate=5:1 to 1:1) to give tert-butyl 2-(1-benzofuran-7-yl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (360 mg, 0.600 mmol, 94% yield) as a yellow oil.

LC-MS (Method 16): Rₜ=1.029 min; MS (ESIpos): m/z=600.3 [M+H]⁺.

### Intermediate 285

### {4-[2-(1-benzofuran-7-yl)-4,5,6,7-tetrahydropyrazolo[1,5-a]pyrazin-3-yl]-3-methyl-1H-pyrrolo[2,3-b]pyridin-1-yl}methanol trifluoroacetate (1/1)

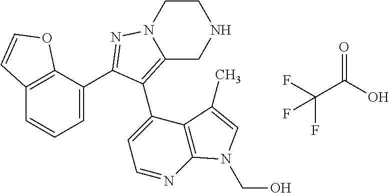 [SMILES: Cc1cn(CO)c2nccc(-c3c(-c4cccc5ccoc45)nn4c3CNCC4)c12 ; O=C(O)C(F)(F)F]

To a solution of tert-butyl 2-(1-benzofuran-7-yl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (360 mg, 0.600 mmol, Intermediate 284) in dichloromethane (8 ml) was added trifluoroacetic acid (4 ml) at 25° C. The mixture was stirred at 25° C. for 4 hours. The reaction mixture was concentrated under reduced pressure to give {4-[2-(1-benzofuran-7-yl)-4,5,6,7-tetrahydropyrazolo[1,5-a]pyrazin-3-yl]-3-methyl-1H-pyrrolo[2,3-b]pyridin-1-yl}methanol (500 mg, 1.25 mmol, 208% yield) as a yellow oil.

LC-MS (Method 16): Rₜ=0.466 min; MS (ESIpos): m/z=400.2 [M+H]⁺.

### Intermediate 286

### 2-(1-benzofuran-7-yl)-3-(3-methyl-1H-pyrrolo[2,3-b]pyridin-4-yl)-4,5,6,7-tetrahydropyrazolo[1,5-a]pyrazine

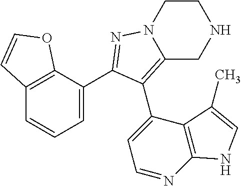 [SMILES: Cc1c[nH]c2nccc(-c3c(-c4cccc5ccoc45)nn4c3CNCC4)c12]

To a solution of {4-[2-(1-benzofuran-7-yl)-4,5,6,7-tetrahydropyrazolo[1,5-a]pyrazin-3-yl]-3-methyl-1H-pyrrolo[2,3-b]pyridin-1-yl}methanol (500 mg, 1.25 mmol, Intermediate 285) in tetrahydrofuran (5 ml) was added sodium hydroxide (2 M solution in water, 5 ml) at 20° C. The mixture was stirred at 20° C. for 4 hours. The reaction mixture was poured into water and extracted with ethyl acetate. The combined organic layers were washed with brine, dried over anhydrous sodium sulfate, filtered and the filtrate was concentrated under reduced pressure to give a residue. The residue was purified by silica gel column chromatography (dichloridemethane:methanol=1:0 to 10:1) to give 2-(1-benzofuran-7-yl)-3-(3-methyl-1H-pyrrolo[2,3-b]pyridin-4-yl)-4,5,6,7-tetrahydropyrazolo[1,5-a]pyrazine (200 mg, 0.541 mmol, 43% yield) as a white solid.

### Intermediate 287

### tert-butyl 3-(pyridin-4-yl)-2-[6-(trifluoromethyl)pyridin-3-yl]-6,7-dihydropyrazolo[1,5-a]pyrazine-5(4H)-carboxylate

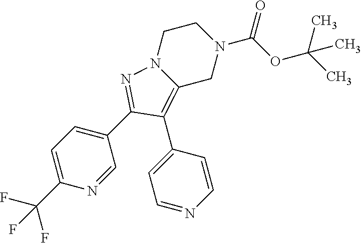 [SMILES: CC(C)(C)OC(=O)N1CCn2nc(-c3ccc(C(F)(F)F)nc3)c(-c3ccncc3)c2C1]

tert-butyl 2-bromo-3-(pyridin-4-yl)-6,7-dihydropyrazolo[1,5-a]pyrazine-5(4H)-carboxylate (115 mg, 303 μmol, Intermediate 68), was dissolved in 1,4-dioxane (1.3 ml) and degassed with nitrogen. 5-(4,4,5,5-tetramethyl-1,3,2-dioxaborolan-2-yl)-2-(trifluoromethyl)pyridine (82.8 mg, 303 μmol), sodium carbonate (64.3 mg, 606 μmol; CAS-RN: [497-19-8]), tert-butyl 2-bromo-3-(pyridin-4-yl)-6,7-dihydropyrazolo[1,5-a]pyrazine-5(4H)-carboxylate (115 mg, 303 μmol), PdCl2(dppf) (22.2 mg, 30.3 μmol; CAS-RN: [72287-26-4]) and degassed water (390 μl) were added, and the vessel was sealed under nitrogen and the mixture was stirred at 110° C. overnight. A solution of sodium carbonate (sat. aqueous) was added, and the mixture extracted with ethyl acetate, the organic phase dried over sodium sulfate and concentrated under reduced pressure. The crude product was dissolved in a small amount of ethyl acetate, and flushed through a 2 g silica column, with washing with additional ethyl acetate until the product had eluted. The fractions were combined and concentrated under reduced pressure yielding the crude title compound. (150 mg, 70% product by UV)

LC-MS (Method 2): Rₜ=1.46 min; MS (ESIpos): m/z=446 [M+H]⁺

¹H-NMR (400 MHz, DMSO-d6) δ [ppm]: 1.065 (16.00), 1.172 (0.65), 1.419 (2.18), 1.987 (1.30), 3.941 (2.75), 4.282 (0.42), 4.688 (0.71), 7.227 (0.78), 7.231 (0.48), 7.238 (0.48), 7.242 (0.79), 7.917 (0.49), 8.584 (0.78), 8.588 (0.47), 8.596 (0.45), 8.599 (0.75).

### Intermediate 288

### tert-butyl 2-phenyl-3-(pyridin-4-yl)-6,7-dihydropyrazolo[1,5-a]pyrazine-5(4H)-carboxylate

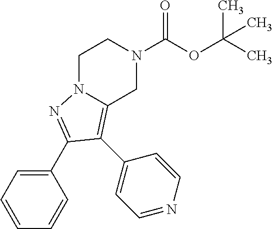 [SMILES: CC(C)(C)OC(=O)N1CCn2nc(-c3ccccc3)c(-c3ccncc3)c2C1]

Analogously to Intermediate 287, tert-butyl 2-phenyl-3-(pyridin-4-yl)-6,7-dihydropyrazolo[1,5-a]pyrazine-5(4H)-carboxylate was produced from tert-butyl 2-bromo-3-(pyridin-4-yl)-6,7-dihydropyrazolo[1,5-a]pyrazine-5(4H)-carboxylate (Intermediate 68) and phenylboronic acid.

LC-MS (Method 2): Rₜ=1.20 min; MS (ESIpos): m/z=377 [M+H]⁺

¹H-NMR (400 MHz, DMSO-d6) δ [ppm]: 1.418 (11.22), 1.444 (2.20), 2.518 (2.61), 2.523 (1.58), 3.319 (0.48), 3.883 (1.21), 3.896 (2.09), 3.910 (1.34), 4.204 (1.61), 4.218 (2.49), 4.232 (1.32), 4.670 (3.82), 5.760 (1.93), 7.135 (4.54), 7.138 (2.71), 7.145 (2.70), 7.150 (4.60), 7.304 (0.42), 7.321 (1.08), 7.343 (16.00), 7.367 (0.65), 7.386 (0.52), 7.450 (0.42), 7.469 (0.77), 7.487 (0.48), 7.653 (0.65), 7.670 (0.62), 7.674 (0.48), 7.765 (0.49), 7.768 (0.51), 7.785 (0.49), 8.032 (1.56), 8.519 (4.20), 8.523 (2.47), 8.530 (2.49), 8.534 (4.05), 9.328 (0.40).

### Intermediate 289

### 3-(pyridin-4-yl)-2-[6-(trifluoromethyl)pyridin-3-yl]-4,5,6,7-tetrahydropyrazolo[1,5-a]pyrazin-5-ium chloride

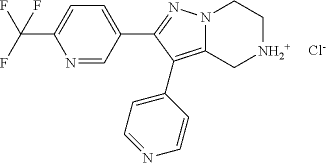 [SMILES: FC(F)(F)c1ccc(-c2nn3c(c2-c2ccncc2)C[NH2+]CC3)cn1 ; [Cl-]]

tert-butyl 3-(pyridin-4-yl)-2-[6-(trifluoromethyl)pyridin-3-yl]-6,7-dihydropyrazolo[1,5-a]pyrazine-5(4H)-carboxylate (150 mg, 337 μmol, Intermediate 287) was dissolved in dichloromethane (10 ml) under an atmosphere of nitrogen. hydrochloric acid (4 M solution in dioxane, 1.7 ml, 4.0 M, 6.7 mmol) was added and the mixture was stirred overnight. The mother liquid was decanted, the the sticky solid dried under vacuum and used without further purification yielding the crude title compound.

LC-MS (Method 2): Rₜ=0.88 min; MS (ESIpos): m/z=346 [M]⁺

### Intermediate 290

### 2-phenyl-3-(pyridin-4-yl)-4,5,6,7-tetrahydropyrazolo[1,5-a]pyrazin-5-ium chloride

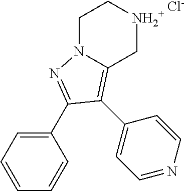 [SMILES: c1ccc(-c2nn3c(c2-c2ccncc2)C[NH2+]CC3)cc1 ; [Cl-]]

Analogously to Intermediate 289, 2-phenyl-3-(pyridin-4-yl)-4,5,6,7-tetrahydropyrazolo[1,5-a]pyrazin-5-ium chloride was produced from tert-butyl 2-phenyl-3-(pyridin-4-yl)-6,7-dihydropyrazolo[1,5-a]pyrazine-5(4H)-carboxylate (Intermediate 288).

LC-MS (Method 2): Rₜ=0.82 min; MS (ESIpos): m/z=277 [M]⁺

### Intermediate 291

### tert-butyl 2-(4-chloro-3-ethylphenyl)-3-(pyridin-4-yl)-6,7-dihydropyrazolo[1,5-a]pyrazine-5(4H)-carboxylate

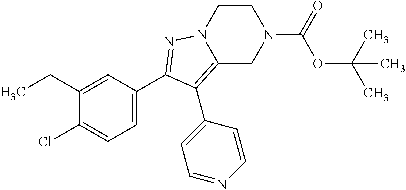 [SMILES: CCc1cc(-c2nn3c(c2-c2ccncc2)CN(C(=O)OC(C)(C)C)CC3)ccc1Cl]

To a solution of tert-butyl 2-bromo-3-(pyridin-4-yl)-6,7-dihydropyrazolo[1,5-a]pyrazine-5(4H)-carboxylate (450 mg, 1.19 mmol, Intermediate 68) in 1,4-dioxane (4.5 ml) and water (1 ml) were added (4-chloro-3-ethylphenyl) boronic acid (263 mg, 1.42 mmol), sodium carbonate (252 mg, 2.37 mmol) and dichloro[1,1-bis(diphenylphosphino) ferrocene]palladium (II) (86.8 mg, 0.12 mmol) at 25° C. The mixture was stirred at 90° C. for 18 hours under nitrogen atmosphere. The mixture was filtered through a pad of celite and the filtrate was concentrated in vacuo. The residue was purified by column chromatography on silica gel (100~200 mesh, petroleum ether:ethyl acetate=5:1) to give tert-butyl 2-(4-chloro-3-ethylphenyl)-3-(pyridin-4-yl)-6,7-dihydropyrazolo[1,5-a]pyrazine-5(4H)-carboxylate (400 mg, 98% purity, 75% yield) as a yellow solid.

LC-MS (Method 16): Rₜ=0.547 min; MS (ESIpos): m/z=439.1 [M+H]⁺

¹H NMR (400 MHz, DMSO-d6) δ [ppm]=7.01 (s, 1H), 4.49 (t, J=6.8 Hz, 2H), 4.01 (q, J=7.2 Hz, 2H), 3.84 (s, 3H), 2.28 (t, J=7.2 Hz, 2H), 2.05-1.96 (m, 2H), 1.65 (t, J=7.2 Hz, 2H).

### Intermediate 292

### 2-(4-chloro-3-ethylphenyl)-3-(pyridin-4-yl)-4,5,6,7-tetrahydropyrazolo[1,5-a]pyrazine

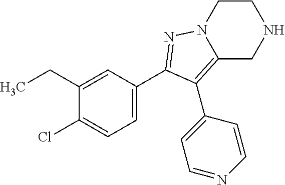 [SMILES: CCc1cc(-c2nn3c(c2-c2ccncc2)CNCC3)ccc1Cl]

To a solution of tert-butyl 2-(4-chloro-3-ethylphenyl)-3-(pyridin-4-yl)-6,7-dihydropyrazolo[1,5-a]pyrazine-5(4H)-carboxylate (390 mg, 871 μmol, Intermediate 292) in ethyl acetate (5.0 ml) was added hydrochloric acid (5 ml, 4M in ethyl acetate) at 25° C. The mixture was stirred at 25° C. for 3 hours. The reaction mixture was concentrated in vacuo to give 2-(4-chloro-3-ethylphenyl)-3-(pyridin-4-yl)-4,5,6,7-tetrahydropyrazolo[1,5-a]pyrazine (300 mg, 94% purity, 86% yield) as a pale yellow solid.

LC-MS (Method 16): Rₜ=0.508 min; MS (ESIpos): m/z=339.1 [M+H]⁺.

### Intermediate 293

### tert-butyl 2-[4-chloro-2-(hydroxymethyl)phenyl]-3-(pyridin-4-yl)-6,7-dihydropyrazolo[1,5-a]pyrazine-5(4H)-carboxylate

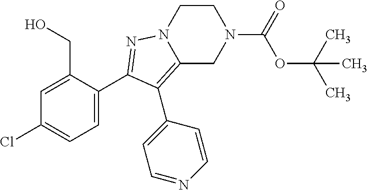 [SMILES: CC(C)(C)OC(=O)N1CCn2nc(-c3ccc(Cl)cc3CO)c(-c3ccncc3)c2C1]

To a solution of tert-butyl 2-bromo-3-(pyridin-4-yl)-6,7-dihydropyrazolo[1,5-a]pyrazine-5(4H)-carboxylate (450 mg, 1.13 mmol, Intermediate 68) in 1,4-dioxane (5.0 ml) and water (1.0 ml) were added [4-chloro-2-(hydroxymethyl)phenyl]boronic acid (252 mg, 1.35 mmol), sodium carbonate (239 mg, 2.25 mmol) and dichloro[1,1-bis(diphenylphosphino) ferrocene]palladium (II) (82.5 mg, 113 μmol) at 25° C. The reaction mixture was stirred at 90° C. for 18 hours under nitrogen atmosphere. The mixture was filtered through a pad of celite and the filtrate was concentrated in vacuo to give a residue. The residue was purified by column chromatography on silica gel (100~200 mesh, =petroleum ether:ethyl acetate 5) to give tert-butyl 2-[4-chloro-2-(hydroxymethyl)phenyl]-3-(pyridin-4-yl)-6,7-dihydropyrazolo[1,5-a]pyrazine-5(4H)-carboxylate (500 mg, 92% purity, 92% yield) as a yellow solid.

LC-MS (Method 16): Rₜ=0.469 min; MS (ESIpos): m/z=441.1 [M+H]⁺.

### Intermediate 294

### {5-chloro-2-[3-(pyridin-4-yl)-4,5,6,7-tetrahydropyrazolo[1,5-a]pyrazin-2-yl]phenyl}methanol

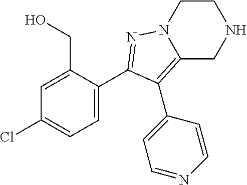 [SMILES: OCc1cc(Cl)ccc1-c1nn2c(c1-c1ccncc1)CNCC2]

To a solution of tert-butyl 2-[4-chloro-2-(hydroxymethyl)phenyl]-3-(pyridin-4-yl)-6,7-dihydropyrazolo[1,5-a]pyrazine-5(4H)-carboxylate (460 mg, 991 μmol, Intermediate 293) in water (5) and dioxane (5 ml) was added sulfuric acid (1 ml) at 20° C. The mixture was stirred at 90° C. for 18 hours. pH of the reaction mixture was adjusted with saturated sodium carbonate aqueous solution to pH=7-8, then the residue was extracted with ethyl acetate. The combined organic phase was washed with brine, dried over anhydrous sodium sulfate, filtered and concentrated in vacuo to give {5-chloro-2-[3-(pyridin-4-yl)-4,5,6,7-tetrahydropyrazolo[1,5-a]pyrazin-2-yl]phenyl}methanol (300 mg, 89% purity, 79% yield) as a pale yellow solid.

LC-MS (Method 16): Rₜ=0.209 min; MS (ESIpos): m/z=341.1 [M+H]⁺.

### Intermediate 295

### tert-butyl 2-(1-benzothiophen-5-yl)-3-(pyridin-4-yl)-6,7-dihydropyrazolo[1,5-a]pyrazine-5(4H)-carboxylate

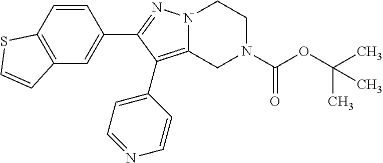 [SMILES: CC(C)(C)OC(=O)N1CCn2nc(-c3ccc4sccc4c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (300 mg, 791 μmol, Intermediate 68) and 2-(1-benzothiophen-5-yl)-4,4,5,5-tetramethyl-1,3,2-dioxaborolane (309 mg, 1.19 mmol) in 1,4-dioxane (5 ml) and water (1 ml) were added (1,1′-bis(diphenylphosphino) ferrocene) palladium (II) dichloride (57.9 mg, 79.1 μmol) and sodium carbonate (168 mg, 1.58 mmol) at 25° C. The mixture was degassed with nitrogen and stirred at 90° C. for 16 hours. The reaction mixture was concentrated under vacuum to give a crude product and the crude product was purified by silica gel column chromatography (petroleum ether:ethyl acetate=1:0 to 1:1) to give tert-butyl 2-(1-benzothiophen-5-yl)-3-(pyridin-4-yl)-6,7-dihydropyrazolo[1,5-a]pyrazine-5(4H)-carboxylate (260 mg, 601 μmol, 76% yield) as a yellow solid.

LC-MS (Method 16): Rₜ=0.849 min; MS (ESIpos): m/z=433.2 [M+H]⁺.

### Intermediate 296

### 2-(1-benzothiophen-5-yl)-3-(pyridin-4-yl)-4,5,6,7-tetrahydropyrazolo[1,5-a]pyrazine hydrogen chloride

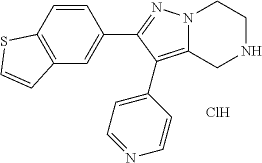 [SMILES: c1cc(-c2c(-c3ccc4sccc4c3)nn3c2CNCC3)ccn1 ; Cl]

To a solution of tert-butyl 2-(1-benzothiophen-5-yl)-3-(pyridin-4-yl)-6,7-dihydropyrazolo[1,5-a]pyrazine-5(4H)-carboxylate (260 mg, 601 μmol, Intermediate 209) in ethyl acetate (10 ml) was added hydrochloric acid (3 ml, 4M in ethyl acetate) at 20° C. and the mixture was stirred at 20° C. for 1 hour. The reaction mixture was concentrated under vacuum to give 2-(1-benzothiophen-5-yl)-3-(pyridin-4-yl)-4,5,6,7-tetrahydropyrazolo[1,5-a]pyrazine hydrogen chloride (220 mg, 596 μmol, 99% yield) as a yellow solid.

LC-MS (Method 15): Rₜ=0.675 min; MS (ESIpos): m/z=333.1 [M+H]⁺

### Intermediate 297

### 2-(4-chloro-3-fluorophenyl)-6,7-dihydropyrazolo[1,5-a]pyrazin-4 (5H)-one

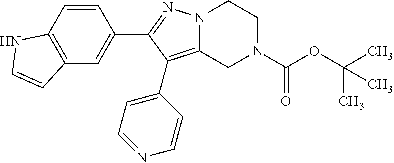 [SMILES: CC(C)(C)OC(=O)N1CCn2nc(-c3ccc4[nH]ccc4c3)c(-c3ccncc3)c2C1]

To a solution of 2-bromo-3-(pyridin-4-yl)-6,7-dihydropyrazolo[1,5-a]pyrazine-5(4H)-carboxylate (400 mg, 1.05 mmol, Intermediate 68) and 1H-indol-5-ylboronic acid (255 mg, 1.58 mmol) in 1,4-dioxane (8.0 ml) and water (2.0 ml) were added sodium carbonate (224 mg, 2.11 mmol) and dichloro[1,1-bis(diphenylphosphino) ferrocene]palladium (II) (77.2 mg, 0.105 mmol) at 20° C. The mixture was degassed with nitrogen and stirred at 90° C. for 16 hours. The reaction solution was poured into water and extracted with ethyl acetate. The combined organic layers were dried over anhydrous sodium sulfate, filtered and the filtrate was concentrated in vacuo to give tert-butyl 2-(1H-indol-5-yl)-3-(pyridin-4-yl)-6,7-dihydropyrazolo[1,5-a]pyrazine-5(4H)-carboxylate (400 mg, 0.962 mmol, 91% yield) as a brown solid.

LC-MS (Method 16): Rₜ=0.710 min; MS (ESIpos): m/z=415.2 [M+H]⁺

### Intermediate 298

### 2-(1H-indol-5-yl)-3-(pyridin-4-yl)-4,5,6,7-tetrahydropyrazolo[1,5-a]pyrazine hydrochloride (1:1)

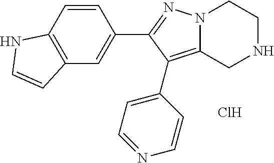 [SMILES: c1cc(-c2c(-c3ccc4[nH]ccc4c3)nn3c2CNCC3)ccn1 ; Cl]

To a solution of 2-(1H-indol-5-yl)-3-(pyridin-4-yl)-6,7-dihydropyrazolo[1,5-a]pyrazine-5(4H)-carboxylate (400 mg, 0.963 mmol, Intermediate 297) in ethyl acetate (15 ml) was added hydrochloric acid (10 ml, 4 M in ethyl acetate) at 20° C. The reaction mixture was stirred at 20° C. for 1 hour. The reaction solution was filtered and washed with ethyl acetate, the solid cake was collected and dried under vacuum to give 2-(1H-indol-5-yl)-3-(pyridin-4-yl)-4,5,6,7-tetrahydropyrazolo[1,5-a]pyrazine hydrochloride (1:1) (300 mg, 0.852 mmol, 88% yield) as a brown oil.

LC-MS (Method 16): R₁=0.123 min; MS (ESIpos): m/z=315.1 [M+H]⁺.

### Intermediate 299

### tert-butyl 2-(1-benzofuran-5-yl)-3-(pyridin-4-yl)-6,7-dihydropyrazolo[1,5-a]pyrazine-5(4H)-carboxylate

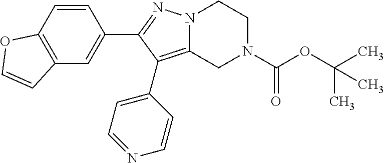 [SMILES: CC(C)(C)OC(=O)N1CCn2nc(-c3ccc4occc4c3)c(-c3ccncc3)c2C1]

To a solution of 2-bromo-3-(pyridin-4-yl)-6,7-dihydropyrazolo[1,5-a]pyrazine-5(4H)-carboxylate (400 mg, 1.05 mmol, Intermediate 68) and 1H-indol-5-ylboronic acid (255 mg, 1.58 mmol) in 1,4-dioxane (8.0 ml) and water (2.0 ml) were added sodium carbonate (224 mg, 2.11 mmol) and dichloro[1,1-bis(diphenylphosphino) ferrocene]palladium (II) (154 mg, 0.211 mmol) at 20° C. The mixture was degassed with nitrogen and stirred at 90° C. for 16 hours. The reaction solution was poured into water and extracted with ethyl acetate. The combined organic layers were dried over anhydrous sodium sulfate, filtered and the filtrate was concentrated in vacuo to give tert-butyl 2-(1-benzofuran-5-yl)-3-(pyridin-4-yl)-6,7-dihydropyrazolo[1,5-a]pyrazine-5(4H)-carboxylate (400 mg, 0.960 mmol, 91% yield) as a brown solid.

LC-MS (Method 14): Rₜ=0.628 min; MS (ESIpos): m/z=416.1 [M+H]⁺

### Intermediate 300

### 2-(1-benzofuran-5-yl)-3-(pyridin-4-yl)-4,5,6,7-tetrahydropyrazolo[1,5-a]pyrazine hydrochloride (1:1)

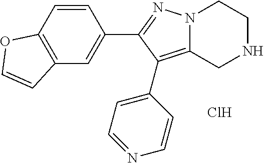 [SMILES: c1cc(-c2c(-c3ccc4occc4c3)nn3c2CNCC3)ccn1 ; Cl]

To a solution of tert-butyl 2-(1-benzofuran-5-yl)-3-(pyridin-4-yl)-6,7-dihydropyrazolo[1,5-a]pyrazine-5(4H)-carboxylate (400 mg, 0.960 mmol, Intermediate 299) in ethyl acetate (10 ml) was added hydrochloric acid (4.0 ml, 4M in ethyl acetate) at 20° C. The reaction mixture was stirred at 20° C. for 1 hour. The reaction mixture was filtered and washed with ethyl acetate. The solid cake was collected and dried under vacuum to give 2-(1-benzofuran-5-yl)-3-(pyridin-4-yl)-4,5,6,7-tetrahydropyrazolo[1,5-a]pyrazine hydrochloride (1:1) (300 mg, 0.850 mmol, 88% yield) as a brown solid.

LC-MS (Method 14): Rₜ=0.500 min; MS (ESIpos): m/z=316.1 [M+H]⁺.

### Intermediate 301

### tert-butyl 2-(1H-indol-6-yl)-3-(pyridin-4-yl)-6,7-dihydropyrazolo[1,5-a]pyrazine-5(4H)-carboxylate

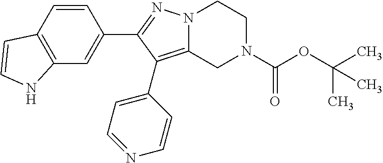 [SMILES: CC(C)(C)OC(=O)N1CCn2nc(-c3ccc4cc[nH]c4c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.05 mmol, Intermediate 68) and 1H-indol-6-ylboronic acid (255 mg, 1.58 mmol) in 1,4-dioxane (8.0 mL) and water (8.0 mL) were added dichloro[1,1-bis(diphenylphosphino) ferrocene]palladium (II) (77.2 mg, 0.105 mmol) and sodium carbonate (224 mg, 2.11 mmol) at 20° C. The mixture was degassed with nitrogen and stirred at 90° C. for 16 hours. The reaction mixture was poured into water and extracted with ethyl acetate. The combined organic layers were dried over anhydrous sodium sulfate, filtered and the filtrate was concentrated in vacuo to give tert-butyl 2-(1H-indol-6-yl)-3-(pyridin-4-yl)-6,7-dihydropyrazolo[1,5-a]pyrazine-5(4H)-carboxylate (400 mg, 0.962 mmol, 91% yield) as a yellow oil.

LC-MS (Method 16): Rₜ=0.781 min; MS (ESIpos): m/z=415.2 [M+H]+

### Intermediate 302

### 2-(1H-indol-6-yl)-3-(pyridin-4-yl)-4,5,6,7-tetrahydropyrazolo[1,5-a]pyrazine hydrochloride (1:1)

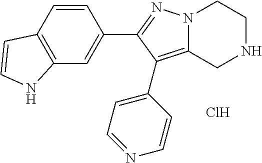 [SMILES: c1cc(-c2c(-c3ccc4cc[nH]c4c3)nn3c2CNCC3)ccn1 ; Cl]

To a solution of tert-butyl 2-(1H-indol-6-yl)-3-(pyridin-4-yl)-6,7-dihydropyrazolo[1,5-a]pyrazine-5(4H)-carboxylate (400 mg, 0.963 mmol, Intermediate 301) in ethyl acetate (5 ml) was added hydrochloric acid (5.0 ml, 4M in ethyl acetate) at 20° C. The mixture was stirred at 20° C. for 1 hour. The reaction mixture was filtered and washed with ethyl acetate. The solid cake was collected and dried under vacuum to give 2-(1H-indol-6-yl)-3-(pyridin-4-yl)-4,5,6,7-tetrahydropyrazolo[1,5-a]pyrazine hydrochloride (1:1) (300 mg, 0.852 mmol, 88% yield) as a brown solid.

LC-MS (Method 16): Rₜ=0.359 min; MS (ESIpos): m/z=315.1 [M+H]⁺

### Intermediate 303

### 6-bromo-1-{[2-(trimethylsilyl) ethoxy]methyl}-1H-benzotriazole

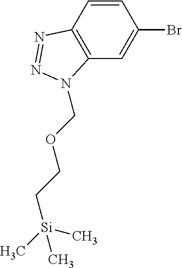 [SMILES: C[Si](C)(C)CCOCn1nnc2ccc(Br)cc21]

To a solution of 6-bromo-1H-benzotriazole (1.00 g, 5.05 mmol) in tetrahydrofuran (10 ml) was added sodium hydride (303 mg, 7.57 mmol, 60% purity) at 0° C. in portions and the mixture was stirred at 25° C. for 1 hour. To this mixture was added trimethylsilylethoxymethoxy chloride (1.38 g, 7.57 mmol) at 20° C. and the resulting mixture was stirred at 20° C. for 1 hour. The reaction mixture was poured into water and extracted with ethyl acetate. Combined extracts were dried over anhydrous sodium sulfate, filtered and the filtrate was concentrated under vacuum to give 6-bromo-1-{[2-(trimethylsilyl) ethoxy]methyl}-1H-benzotriazole (1.00 g, 3.05 mmol, 60% yield) as a brown liquid.

LC-MS (Method 16): Rₜ=1.066 min; MS (ESIpos): m/z=300.1 [M+2+H]⁺.

### Intermediate 304

### 6-(4,4,5,5-tetramethyl-1,3,2-dioxaborolan-2-yl)-1-{[2-(trimethylsilyl) ethoxy]methyl}-1H-benzotriazole

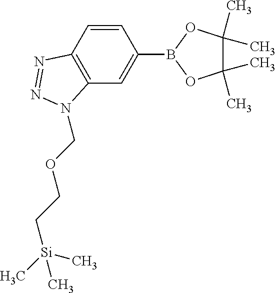

To a solution of 6-bromo-1-{[2-(trimethylsilyl) ethoxy]methyl}-1H-benzotriazole (1.00 g, 3.05 mmol, Intermediate 303) and 4,4,4′,4′,5,5,5′,5′-octamethyl-2,2′-bi-1,3,2-dioxaborolane (1.55 g, 6.09 mmol) in 1,4-dioxane (20 ml) were added (1,1′-bis(diphenylphosphino) ferrocene) palladium (II) dichloride (223 mg, 305 μmol) and potassium acetate (598 mg, 6.09 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=10:1) to give 6-(4,4,5,5-tetramethyl-1,3,2-dioxaborolan-2-yl)-1-{[2-(trimethylsilyl) ethoxy]methyl}-1H-benzotriazole (900 mg, 2.40 mmol, 79% yield) as a yellow oil.

LC-MS (Method 16): Rₜ=1.118 min; MS (ESIpos): m/z=376.2 [M+H]⁺.

### Intermediate 305

### tert-butyl 3-(pyridin-4-yl)-2-(1-{[2-(trimethylsilyl) ethoxy]methyl}-1H-benzotriazol-6-yl)-6,7-dihydropyrazolo[1,5-a]pyrazine-5(4H)-carboxylate

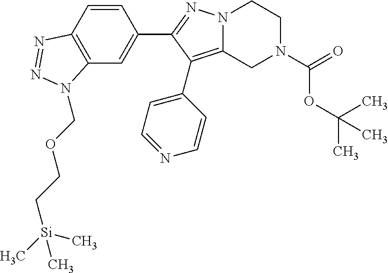 [SMILES: CC(C)(C)OC(=O)N1CCn2nc(-c3ccc4nnn(COCC[Si](C)(C)C)c4c3)c(-c3ccncc3)c2C1]

To a solution of tert-butyl 2-bromo-3-(pyridin-4-yl)-6,7-dihydropyrazolo[1,5-a]pyrazine-5(4H)-carboxylate (400 mg, 1.06 mmol, Intermediate 68) and 6-(4,4,5,5-tetramethyl-1,3,2-dioxaborolan-2-yl)-1-{[2-(trimethylsilyl) ethoxy]methyl}-1H-benzotriazole (396 mg, 1.06 mmol, Intermediate 304) in 1,4-dioxane (10 ml) and water (2 ml) were added (1,1′-bis(diphenylphosphino) ferrocene) palladium (II) dichloride (77.2 mg, 106 μmol) and sodium carbonate (224 mg, 2.11 mmol) at 25° C. The mixture was degassed with nitrogen and then stirred at 90° C. for 16 hours. The reaction mixture was concentrated under vacuum to give a crude product. The crude product was purified by silica gel column chromatography (petroleum ether:ethyl acetate=1:0 to 0:1) to give tert-butyl 3-(pyridin-4-yl)-2-(1-{[2-(trimethylsilyl) ethoxy]methyl}-1H-benzotriazol-6-yl)-6,7-dihydropyrazolo[1,5-a]pyrazine-5(4H)-carboxylate (420 mg, 767 μmol, 73% yield) as a brown oil.

LC-MS (Method 16): R₁=0.913 min; MS (ESIpos): m/z=548.3 [M+H]⁺.

### Intermediate 306

### 6-[3-(pyridin-4-yl)-4,5,6,7-tetrahydropyrazolo[1,5-a]pyrazin-2-yl]-1H-benzotriazole hydrogen chloride

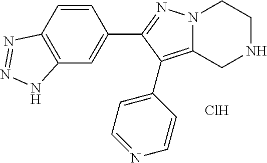 [SMILES: c1cc(-c2c(-c3ccc4nn[nH]c4c3)nn3c2CNCC3)ccn1 ; Cl]

To a solution of tert-butyl 3-(pyridin-4-yl)-2-(1-{[2-(trimethylsilyl) ethoxy]methyl}-1H-benzotriazol-6-yl)-6,7-dihydropyrazolo[1,5-a]pyrazine-5(4H)-carboxylate (400 mg, 730 μmol, Intermediate 305) in ethyl acetate (6 ml) was added hydrochloric acid (18 ml, 72.0 mmol, 4.0 M in ethyl acetate) at 25° C. and the mixture was stirred at 25° C. for 1 hour. The precipitate was collected by filtration, washed with ethyl acetate and dried under vacuum to give 6-[3-(pyridin-4-yl)-4,5,6,7-tetrahydropyrazolo[1,5-a]pyrazin-2-yl]-1H-benzotriazole hydrogen chloride (200 mg, 565 μmol, 77% yield) as a yellow solid.

LC-MS (Method 20): Rₜ=0.264 min; MS (ESIpos): m/z=318.1 [M+H]⁺.

### Intermediate 307

### (2RS)-1-amino-3-(benzyloxy) propan-2-ol

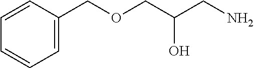 [SMILES: NCC(O)COCc1ccccc1]

The mixture of 2-[(benzyloxy)methyl]oxirane (50.0 g, 0.304 mmol) and ammonium hydroxide (381 g, 3.04 mol) was stirred at 20° C. for 16 hours. The reaction mixture was filtered and the filtrate was concentrated in vacuo to give (2RS)-1-amino-3-(benzyloxy) propan-2-ol (18.0 g, 0.099 mol, 33% yield) as a white solid.

LC-MS (Method 16): Rₜ=0.368 min; MS (ESIpos): m/z=182.3 [M+H]⁺.

### Intermediate 308

### tert-butyl [(2RS)-3-(benzyloxy)-2-hydroxypropyl]carbamate

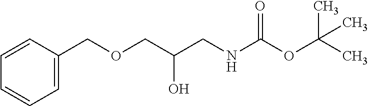 [SMILES: CC(C)(C)OC(=O)NCC(O)COCc1ccccc1]

To a solution of (2RS)-1-amino-3-(benzyloxy) propan-2-ol (18.0 g, 0.099 mol) and triethylamine (15.1 g, 0.149 mol) in dichloromethane (180 ml) was added di-tert-butyl dicarbonate (28.2 g, 0.129 mol) at 20° C. The mixture was stirred at 20° C. for 3 hours. The reaction mixture was concentrated in vacuo to give a residue and the residue was purified by column chromatography on silica gel (petroleum ether:ethyl acetate=20:1 to 2:1) to give tert-butyl [(2RS)-3-(benzyloxy)-2-hydroxypropyl]carbamate (22.0 g, 0.0782 mol, 79% yield) as a colorless oil.

LC-MS (Method 16): Rₜ=0.742 min; MS (ESIpos): m/z=182.1 [(M−100)+H]⁺

### Intermediate 309

### ethyl 1-{(2RS)-1-(benzyloxy)-3-[(tert-butoxycarbonyl)amino]propan-2-yl}-3-bromo-1H-pyrazole-5-carboxylate

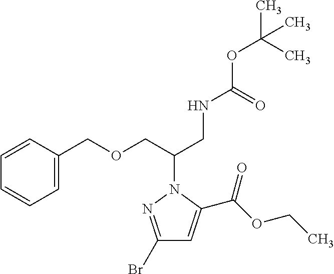 [SMILES: CCOC(=O)c1cc(Br)nn1C(CNC(=O)OC(C)(C)C)COCc1ccccc1]

To a solution of tert-butyl [(2RS)-3-(benzyloxy)-2-hydroxypropyl]carbamate (22.0 g, 0.0782 mol, Intermediate 308) and ethyl 5-bromo-1H-pyrazole-3-carboxylate (17.1 g, 0.0782 mol) in tetrahydrofuran (660 ml) were added diisopropyl azodicarboxylate (31.6 g, 0.156 mol) and triphenylphosphine (41.0 g, 0.156 mol) at 20° C. The mixture was stirred at 20° C. for 16 hours. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1) to give ethyl 1-{(2RS)-1-(benzyloxy)-3-[(tert-butoxycarbonyl)amino]propan-2-yl}-3-bromo-1H-pyrazole-5-carboxylate (27.0 g, 0.0560 mol, 72% yield) as a colourless oil.

LC-MS (Method 16): Rₜ=0.939 min; MS (ESIpos): m/z=426.1 [(M−55)+H]⁺.

### Intermediate 310

### ethyl 1-[(2RS)-1-amino-3-(benzyloxy) propan-2-yl]-3-bromo-1H-pyrazole-5-carboxylate hydrochloride (1:1)

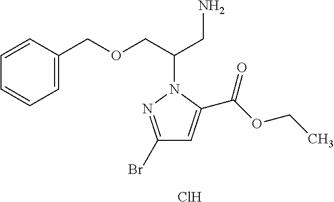 [SMILES: CCOC(=O)c1cc(Br)nn1C(CN)COCc1ccccc1 ; Cl]

To a solution of ethyl 1-{(2RS)-1-(benzyloxy)-3-[(tert-butoxycarbonyl)amino]propan-2-yl}-3-bromo-1H-pyrazole-5-carboxylate (27.0 g, 0.0560 mol, Intermediate 309) in ethyl acetate (162 ml) was added hydrochloric acid (4M in ethyl acetate, 28 ml, 0.112 mol) at 20° C. The mixture was stirred at 20° C. for 5 h. The reaction mixture was filtered and the solid cake was collected to give ethyl 1-[(2RS)-1-amino-3-(benzyloxy) propan-2-yl]-3-bromo-1H-pyrazole-5-carboxylate hydrochloride (1:1) (22.0 g, 0.0525 mol, 94% yield) as a white solid.

LC-MS (Method 16): Rₜ=0.793 min; MS (ESIpos): m/z=368.1 [(M−36)+H]⁺

### Intermediate 311

### (7RS)-7-[(benzyloxy)methyl]-2-bromo-6,7-dihydropyrazolo[1,5-a]pyrazin-4 (5H)-one

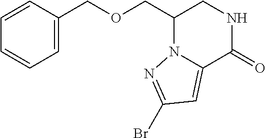 [SMILES: O=C1NCC(COCc2ccccc2)n2nc(Br)cc21]

To a solution of ethyl 1-[(2RS)-1-amino-3-(benzyloxy) propan-2-yl]-3-bromo-1H-pyrazole-5-carboxylate hydrochloride (1:1) (22.0 g, 0.0525 mol, Intermediate 310) in methanol (88 ml) was added triethylamine (15 ml, 0.105 mol) at 20° C. The mixture was stirred at 50° C. for 5 hours. The reaction mixture was concentrated in vacuo to give (7RS)-7-[(benzyloxy)methyl]-2-bromo-6,7-dihydropyrazolo[1,5-a]pyrazin-4 (5H)-one (12.5 g, 0.0372 mol, 71% yield) as a white solid.

LC-MS (Method 16): Rₜ=0.878 min; MS (ESIpos): m/z=336.1 [M+H]⁺.

### Intermediate 312

### (7RS)-7-[(benzyloxy)methyl]-2-(4-fluorophenyl)-6,7-dihydropyrazolo[1,5-a]pyrazin-4 (5H)-one

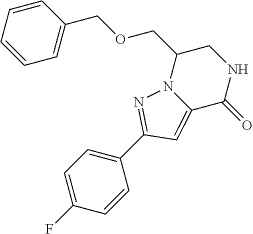 [SMILES: O=C1NCC(COCc2ccccc2)n2nc(-c3ccc(F)cc3)cc21]

To a solution of (7RS)-7-[(benzyloxy)methyl]-2-bromo-6,7-dihydropyrazolo[1,5-a]pyrazin-4 (5H)-one (12.5 g, 0.0372 mol, Intermediate 311) and (4-fluorophenyl) boronic acid (5.46 g, 0.0390 mol) in 1,4-dioxane (300 ml) and water (100 ml) were added [1,1-Bis(diphenylphosphino) ferrocene]dichloropalladium (II) (2.72 g, 0.0372 mol) and sodium carbonate (7.88 g, 0.0744 mol) at 20° C. The mixture was stirred at 90° C. for 16 hours. The reaction mixture was poured into water and extracted with ethyl acetate. The combined organic layers were concentrated under vacuo to give a residue. The residue was triturated with a mixed solvent of petroleum ether and ethyl acetate (3:1, 200 ml) to give (7RS)-7-[(benzyloxy)methyl]-2-(4-fluorophenyl)-6,7-dihydropyrazolo[1,5-a]pyrazin-4 (5H)-one (9.50 g, 0.0270 mol, 73% yield) as a yellow solid.

LC-MS (Method 16): Rₜ=0.945 min; MS (ESIpos): m/z=352.1 [M+H]⁺.

### Intermediate 313

### (7RS)-7-[(benzyloxy)methyl]-2-(4-fluorophenyl)-4,5,6,7-tetrahydropyrazolo[1,5-a]pyrazine

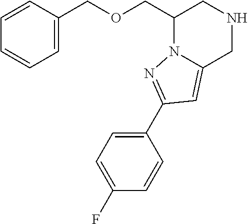 [SMILES: Fc1ccc(-c2cc3n(n2)C(COCc2ccccc2)CNC3)cc1]

To a solution of (7RS)-7-[(benzyloxy)methyl]-2-(4-fluorophenyl)-6,7-dihydropyrazolo[1,5-a]pyrazin-4 (5H)-one (9.50 g, 27.0 mmol, Intermediate 312) in tetrahydrofuran (290 ml) was added lithium aluminum hydride (1M in tetrahydrofuran, 135 ml, 135 mmol) at 0° C. The mixture was stirred at 60° C. for 4 hours. The reaction was quenched with water and 10% sodium hydroxide aqueous solution, and the resulting solution was extracted with ethyl acetate. The combined organic layers were washed with brine, dried over anhydrous sodium sulfate, filtered and the filtrate was concentrated in vacuo to give a residue. The residue was purified by column chromatography on silica gel (petroleum ether:ethyl acetate=30:1 to 3:1) to give (7RS)-7-[(benzyloxy)methyl]-2-(4-fluorophenyl)-4,5,6,7-tetrahydropyrazolo[1,5-a]pyrazine (3.60 g, 10.7 mmol, 39% yield) as a white solid.

LC-MS (Method 16): Rₜ=0.834 min; MS (ESIpos): m/z=338.2 [M+H]⁺

### Intermediate 314

### tert-butyl (7RS)-7-((benzyloxy)methyl)-2-(4-fluorophenyl)-6,7-dihydropyrazolo[1,5-a]pyrazine-5(4H)-carboxylate

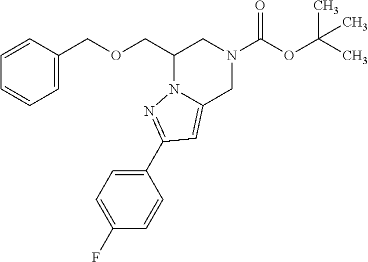 [SMILES: CC(C)(C)OC(=O)N1Cc2cc(-c3ccc(F)cc3)nn2C(COCc2ccccc2)C1]

The solution of (7RS)-7-[(benzyloxy)methyl]-2-(4-fluorophenyl)-4,5,6,7-tetrahydropyrazolo[1,5-a]pyrazine (3.60 g, 10.7 mmol, Intermediate 313) in dichloromethane (49 ml) were added triethylamine (3 ml, 21.3 mmol) and di-tert-butyl dicarbonate (2.79 g, 12.8 mmol) at 20° C. The mixture was stirred at 20° C. for 4 hours. The reaction mixture was concentrated under vacuum to give tert-butyl (7RS)-7-((benzyloxy)methyl)-2-(4-fluorophenyl)-6,7-dihydropyrazolo[1,5-a]pyrazine-5(4H)-carboxylate (3.30 g, 7.54 mmol, 71% yield) as a yellow solid.

LC-MS (Method 16): Rₜ=1.107 min; MS (ESIpos): m/z=438.2 [M+H]⁺

### Intermediate 315

### tert-butyl (7RS)-7-((benzyloxy)methyl)-3-bromo-2-(4-fluorophenyl)-6,7-dihydropyrazolo[1,5-a]pyrazine-5(4H)-carboxylate

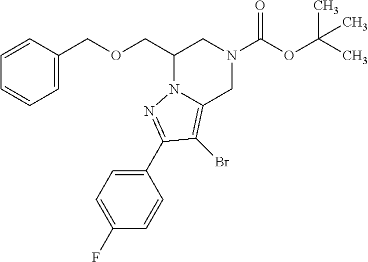 [SMILES: CC(C)(C)OC(=O)N1Cc2c(Br)c(-c3ccc(F)cc3)nn2C(COCc2ccccc2)C1]

To a solution of tert-butyl (7RS)-7-((benzyloxy)methyl)-2-(4-fluorophenyl)-6,7-dihydropyrazolo[1,5-a]pyrazine-5(4H)-carboxylate (3.30 g, 7.54 mmol, Intermediate 314) in N,N-dimethylformamide (36 ml) was added 1-bromopyrrolidine-2,5-dione (1.61 g, 9.05 mmol) at 20° C. The mixture was stirred at 20° C. for 1 hour. The reaction mixture was poured into water and extracted with ethyl acetate. The combined organic layers were washed with brine, dried over anhydrous sodium sulfate, filtered and the filtrate was concentrated in vacuo to give tert-butyl (7RS)-7-((benzyloxy)methyl)-3-bromo-2-(4-fluorophenyl)-6,7-dihydropyrazolo[1,5-a]pyrazine-5(4H)-carboxylate (3.80 g, 7.36 mmol, 98% yield) as a brown solid.

LC-MS (Method 16): Rₜ=1.152 min; MS (ESIpos): m/z=518.2 [M+H]⁺.

### Intermediate 316

### tert-butyl (7RS)-7-[(benzyloxy)methyl]-2-(4-fluorophenyl)-3-(pyridin-4-yl)-6,7-dihydropyrazolo[1,5-a]pyrazine-5(4H)-carboxylate

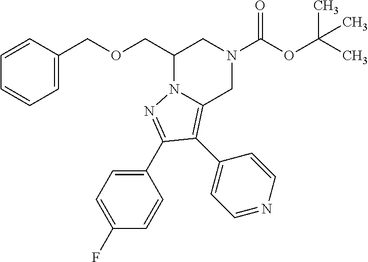 [SMILES: CC(C)(C)OC(=O)N1Cc2c(-c3ccncc3)c(-c3ccc(F)cc3)nn2C(COCc2ccccc2)C1]

To a solution of tert-butyl (7RS)-7-((benzyloxy)methyl)-3-bromo-2-(4-fluorophenyl)-6,7-dihydropyrazolo[1,5-a]pyrazine-5(4H)-carboxylate (3.80 g, 7.36 mmol, Intermediate 315) and pyridin-4-ylboronic acid (1.81 g, 14.7 mmol) in 1,4-dioxane (82 ml) and water (20 ml) were added sodium carbonate and [1,1-bis(diphenylphosphino) ferrocene]dichloropalladium (II) (0.538 g, 0.736 mmol) at 20° C. The mixture was stirred at 80° C. for 16 hours under nitrogen atmosphere. The reaction mixture was poured into water and extracted with ethyl acetate. The combined organic layers were washed with brined, dried over anhydrous sodium sulfate, filtered and concentrated in vacuo to give a crude product. The crude product was purified by column chromatography on silica gel (petroleum ether:ethyl acetate=20:1 to 2:1) to give tert-butyl (7RS)-7-[(benzyloxy)methyl]-2-(4-fluorophenyl)-3-(pyridin-4-yl)-6,7-dihydropyrazolo[1,5-a]pyrazine-5(4H)-carboxylate (3.10 g, 6.02 mmol, 82% yield) as a yellow solid.

LC-MS (Method 16): Rₜ=0.556 min; MS (ESIpos): m/z=515.3 [M+H]⁺

### Intermediate 317

### tert-butyl (7RS)-2-(4-fluorophenyl)-7-(hydroxymethyl)-3-(pyridin-4-yl)-6,7-dihydropyrazolo[1,5-a]pyrazine-5(4H)-carboxylate

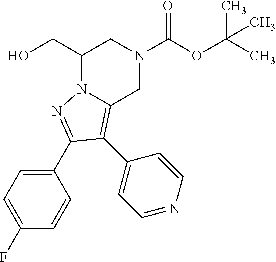 [SMILES: CC(C)(C)OC(=O)N1Cc2c(-c3ccncc3)c(-c3ccc(F)cc3)nn2C(CO)C1]

To a solution of tert-butyl (7RS)-7-[(benzyloxy)methyl]-2-(4-fluorophenyl)-3-(pyridin-4-yl)-6,7-dihydropyrazolo[1,5-a]pyrazine-5(4H)-carboxylate (3.10 g, 6.02 mmol, Intermediate 316) in methanol (34 ml) were added palladium/carbon (0.0641 g, 0.602 mmol) and palladium (II) hydroxide (0.0846 g, 0.602 mmol) at 20° C. The mixture was stirred at 25° C. for 16 hours. The reaction mixture was filtered through a pad of celite and the filtrate was concentrated in vacuo to give tert-butyl (7RS)-2-(4-fluorophenyl)-7-(hydroxymethyl)-3-(pyridin-4-yl)-6,7-dihydropyrazolo[1,5-a]pyrazine-5(4H)-carboxylate (2.00 g, 4.71 mmol, 78% yield) as a yellow oil.

LC-MS (Method 16): Rₜ=0.767 min; MS (ESIpos): m/z=425.2 [M+H]⁺.

### Intermediate 318

### [(7RS)-2-(4-fluorophenyl)-3-(pyridin-4-yl)-4,5,6,7-tetrahydropyrazolo[1,5-a]pyrazin-7-yl]methanol hydrochloride (1:1)

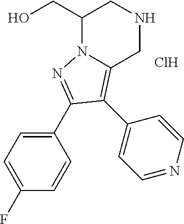 [SMILES: OCC1CNCc2c(-c3ccncc3)c(-c3ccc(F)cc3)nn21 ; Cl]

To a solution of tert-butyl (7RS)-2-(4-fluorophenyl)-7-(hydroxymethyl)-3-(pyridin-4-yl)-6,7-dihydropyrazolo[1,5-a]pyrazine-5(4H)-carboxylate (500 mg, 1.18 mmol, Intermediate 317) in ethyl acetate (1.7 ml) was added hydrochloric acid (4M in ethyl acetate, 10 ml, 1.77 mmol) at 20° C. The mixture was stirred at 20° C. for 1 hour. The reaction mixture was filtered and the solid cake was collected to give [(7RS)-2-(4-fluorophenyl)-3-(pyridin-4-yl)-4,5,6,7-tetrahydropyrazolo[1,5-a]pyrazin-7-yl]methanol hydrochloride (1:1) (380 mg, 1.05 mmol, 89% yield) as a brown solid.

LC-MS (Method 16): Rₜ=0.125 min; MS (ESIpos): m/z=325.1 [M+H]⁺.

### Intermediate 319

### tert-butyl (7RS)-2-(4-fluorophenyl)-7-formyl-3-(pyridin-4-yl)-6,7-dihydropyrazolo[1,5-a]pyrazine-5(4H)-carboxylate

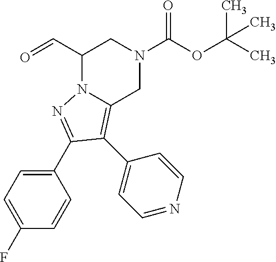 [SMILES: CC(C)(C)OC(=O)N1Cc2c(-c3ccncc3)c(-c3ccc(F)cc3)nn2C(C=O)C1]

To a solution of tert-butyl (7RS)-2-(4-fluorophenyl)-7-(hydroxymethyl)-3-(pyridin-4-yl)-6,7-dihydropyrazolo[1,5-a]pyrazine-5(4H)-carboxylate (2.00 g, 4.71 mmol, Intermediate 317) in dichloromethane (20 ml) was added 1,1,1-triacetoxy-1lambda5,2-benziodoxol-3 (1H)-one (6.00 g, 14.1 mmol) at 25° C. The mixture was stirred at 25° C. for 4 hours. The reaction was quenched with sodium hydrogencarbonate aqueous solution, the resulting mixture was filtered and the filtrate was extracted with ethyl acetate. The combined organic phase was dried over anhydrous sodium sulfate, filtered and concentrated in vacuo to give tert-butyl (7RS)-2-(4-fluorophenyl)-7-formyl-3-(pyridin-4-yl)-6,7-dihydropyrazolo[1,5-a]pyrazine-5(4H)-carboxylate (1.90 g, 4.50 mmol, 95% yield) as a brown oil.

### Intermediate 320

### (7RS)-5-(tert-butoxycarbonyl)-2-(4-fluorophenyl)-3-(pyridin-4-yl)-4,5,6,7-tetrahydropyrazolo[1,5-a]pyrazine-7-carboxylic acid

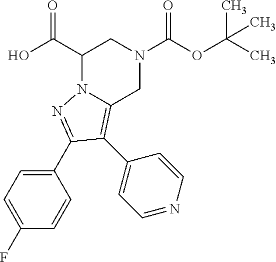 [SMILES: CC(C)(C)OC(=O)N1Cc2c(-c3ccncc3)c(-c3ccc(F)cc3)nn2C(C(=O)O)C1]

To a solution of tert-butyl (7RS)-2-(4-fluorophenyl)-7-formyl-3-(pyridin-4-yl)-6,7-dihydropyrazolo[1,5-a]pyrazine-5(4H)-carboxylate (1.90 g, 4.50 mmol, Intermediate 319) in tert-butanol (114 ml) and water (38 ml) were added potassium dihydrogen phosphate (0.612 g, 4.50 mmol) and sodium chlorite (0.569 g, 6.30 mmol) at 25° C. The mixture was stirred at 25° C. for 1 hour. The reaction mixture was poured into water and extracted with ethyl acetate. The combined organic phase was concentrated in vacuo to give (7RS)-5-(tert-butoxycarbonyl)-2-(4-fluorophenyl)-3-(pyridin-4-yl)-4,5,6,7-tetrahydropyrazolo[1,5-a]pyrazine-7-carboxylic acid (1.70 g, 3.88 mmol, 86% yield) as a brown oil.

LC-MS (Method 20): Rₜ=0.687 min; MS (ESIpos): m/z=437.2 [M−H]:

### Intermediate 321

### tert-butyl (7RS)-7-carbamoyl-2-(4-fluorophenyl)-3-(pyridin-4-yl)-6,7-dihydropyrazolo[1,5-a]pyrazine-5(4H)-carboxylate

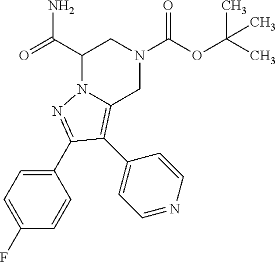 [SMILES: CC(C)(C)OC(=O)N1Cc2c(-c3ccncc3)c(-c3ccc(F)cc3)nn2C(C(N)=O)C1]

To a solution of (7RS)-5-(tert-butoxycarbonyl)-2-(4-fluorophenyl)-3-(pyridin-4-yl)-4,5,6,7-tetrahydropyrazolo[1,5-a]pyrazine-7-carboxylic acid (300 mg, 0.684 mmol, Intermediate 320) and ammonium chloride (73.9 mg, 1.37 mmol) in tetrahydrofuran (8 ml) were added O-(7-azabenzotriazol-1-yl)-N,N,N′,N′-tetramethyluronium hexafluorophosphate (520 mg, 1.37 mmol) and N,N-diisopropylethylamine (265 mg, 2.05 mmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give tert-butyl (7RS)-7-carbamoyl-2-(4-fluorophenyl)-3-(pyridin-4-yl)-6,7-dihydropyrazolo[1,5-a]pyrazine-5(4H)-carboxylate (180 mg, 0.411 mmol, 60% yield) as a brown solid.

LC-MS (Method 20): Rₜ=0.877 min; MS (ESIpos): m/z=436.1 [M−H]:

### Intermediate 322

### (7RS)-2-(4-fluorophenyl)-3-(pyridin-4-yl)-4,5,6,7-tetrahydropyrazolo[1,5-a]pyrazine-7-carboxamide hydrogen chloride (1/1)

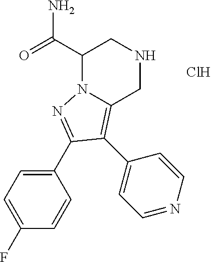 [SMILES: NC(=O)C1CNCc2c(-c3ccncc3)c(-c3ccc(F)cc3)nn21 ; Cl]

To a solution of tert-butyl (7RS)-7-carbamoyl-2-(4-fluorophenyl)-3-(pyridin-4-yl)-6,7-dihydropyrazolo[1,5-a]pyrazine-5(4H)-carboxylate (180 mg, 0.411 mmol, Intermediate 321) in ethyl acetate (12 ml) was added hydrochloric acid (4M in ethyl acetate, 1 ml, 4.11 mmol) at 25° C. The mixture was stirred at 25° C. for 1 hour. The reaction mixture was filtered and the solid cake was collected to give (7RS)-2-(4-fluorophenyl)-3-(pyridin-4-yl)-4,5,6,7-tetrahydropyrazolo[1,5-a]pyrazine-7-carboxamide hydrogen chloride (1/1) (110 mg, 0.294 mmol, 72% yield) as a yellow solid.

LC-MS (Method 20): Rₜ=0.769 min; MS (ESIpos): m/z=338.1 [M+H]⁺.

### Intermediate 323

### tert-butyl (7RS)-2-(4-fluorophenyl)-7-(methylcarbamoyl)-3-(pyridin-4-yl)-6,7-dihydropyrazolo[1,5-a]pyrazine-5(4H)-carboxylate

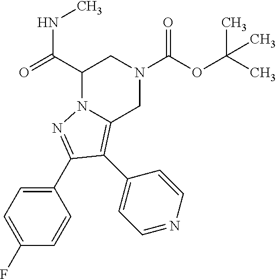 [SMILES: CNC(=O)C1CN(C(=O)OC(C)(C)C)Cc2c(-c3ccncc3)c(-c3ccc(F)cc3)nn21]

To a solution of (7RS)-5-(tert-butoxycarbonyl)-2-(4-fluorophenyl)-3-(pyridin-4-yl)-4,5,6,7-tetrahydropyrazolo[1,5-a]pyrazine-7-carboxylic acid (600 mg, 1.38 mmol, Intermediate 320) and methanamine hydrochloride (1:1) (111 mg, 1.64 mmol) in N,N-dimethylformamide were added 1-[bis(dimethylamino)methylene]-1H-1,2,3-triazolo[4,5-b]pyridinium-3-oxide hexafluorophosphate (1.04 g, 2.74 mmol) and N,N-diisopropylethylamine (531 mg, 4.11 mmol) at 25° C. The mixture was stirred at 25° C. for 16 hours. The reaction mixture was concentrated in vacuo to give a residue. The residue was purified by preparative TLC (ethyl acetate: NH₃·H₂O=100:1) to give tert-butyl (7RS)-2-(4-fluorophenyl)-7-(methylcarbamoyl)-3-(pyridin-4-yl)-6,7-dihydropyrazolo[1,5-a]pyrazine-5(4H)-carboxylate (210 mg, 0.465 mmol, 34% yield) as a yellow solid.

LC-MS (Method 16): Rₜ=0.908 min; MS (ESIpos): m/z=452.2 [M+H]⁺.

### Intermediate 324

### (7RS)-2-(4-fluorophenyl)-N-methyl-3-(pyridin-4-yl)-4,5,6,7-tetrahydropyrazolo[1,5-a]pyrazine-7-carboxamide hydrochloride (1:1)

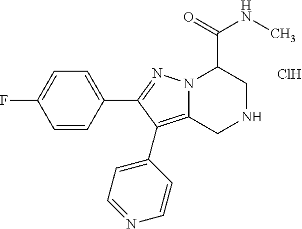 [SMILES: CNC(=O)C1CNCc2c(-c3ccncc3)c(-c3ccc(F)cc3)nn21 ; Cl]

To a solution of tert-butyl (7RS)-2-(4-fluorophenyl)-7-(methylcarbamoyl)-3-(pyridin-4-yl)-6,7-dihydropyrazolo[1,5-a]pyrazine-5(4H)-carboxylate (210 mg, 465 μmol, Intermediate 323) in ethyl acetate (10 ml) was added hydrochloric acid (4M in ethyl acetate, 2 ml, 9.30 mmol) at 25° C. The mixture was stirred at 25° C. for 1 hour. The reaction mixture was filtered and the solid cake was collected to give (7RS)-2-(4-fluorophenyl)-N-methyl-3-(pyridin-4-yl)-4,5,6,7-tetrahydropyrazolo[1,5-a]pyrazine-7-carboxamide hydrochloride (1:1) (120 mg, 309 μmol, 67% yield) as a light yellow solid.

### Intermediate 325

### tert-butyl (7RS)-7-(dimethylcarbamoyl)-2-(4-fluorophenyl)-3-(pyridin-4-yl)-6,7-dihydropyrazolo[1,5-a]pyrazine-5(4H)-carboxylate

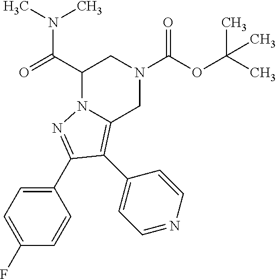 [SMILES: CN(C)C(=O)C1CN(C(=O)OC(C)(C)C)Cc2c(-c3ccncc3)c(-c3ccc(F)cc3)nn21]

To a solution of (7RS)-5-(tert-butoxycarbonyl)-2-(4-fluorophenyl)-3-(pyridin-4-yl)-4,5,6,7-tetrahydropyrazolo[1,5-a]pyrazine-7-carboxylic acid (700 mg, 1.60 mmol, Intermediate 320) and N-methylmethanamine hydrochloride (1:1) (156 mg, 1.92 mmol) in N,N-dimethylformamide were added 1-[bis(dimethylamino)methylene]-1H-1,2,3-triazolo[4,5-b]pyridinium-3-oxide hexafluorophosphate (1.21 3.19 g, mmol) and N,N-diisopropylethylamine (619 mg, 4.79 mmol) at 25° C. The mixture was stirred at 25° C. for 3 hours. The reaction mixture was concentrated in vacuo to give a residue. The residue was purified by preparative TLC (ethyl acetate: NH₃·H₂O=100:1) to give tert-butyl (7RS)-7-(dimethylcarbamoyl)-2-(4-fluorophenyl)-3-(pyridin-4-yl)-6,7-dihydropyrazolo[1,5-a]pyrazine-5(4H)-carboxylate (320 mg, 0.687 mmol, 43% yield) as a yellow solid.

LC-MS (Method 17): Rₜ=0.946 min; MS (ESIpos): m/z=466.2 [M+H]⁺.

### Intermediate 326

### (7RS)-2-(4-fluorophenyl)-N,N-dimethyl-3-(pyridin-4-yl)-4,5,6,7-tetrahydropyrazolo[1,5-a]pyrazine-7-carboxamide hydrochloride (1:1)

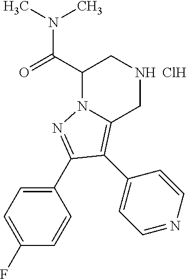 [SMILES: CN(C)C(=O)C1CNCc2c(-c3ccncc3)c(-c3ccc(F)cc3)nn21 ; Cl]

To a solution of tert-butyl (7RS)-7-(dimethylcarbamoyl)-2-(4-fluorophenyl)-3-(pyridin-4-yl)-6,7-dihydropyrazolo[1,5-a]pyrazine-5(4H)-carboxylate (320 mg, 0.687 mmol, Intermediate 325) in ethyl acetate was added hydrochloric acid (4M in ethy acetate, 3 ml, 13.7 mmol) at 25° C. The mixture was stirred at 25° C. for 1 hour. The mixture was filtered and the solid cake was collected to give (7RS)-2-(4-fluorophenyl)-N,N-dimethyl-3-(pyridin-4-yl)-4,5,6,7-tetrahydropyrazolo[1,5-a]pyrazine-7-carboxamide hydrochloride (1:1) (220 mg, 0.547 mmol, 80% yield) as a light yellow solid.

LC-MS (Method 17): Rₜ=0.820 min; MS (ESIpos): m/z=366.2 [M+H]⁺.

### Intermediate 327

### methyl 3-bromo-1-{1-[(tert-butoxycarbonyl)amino]-2-methylpropan-2-yl}-1H-pyrazole-5-carboxylate

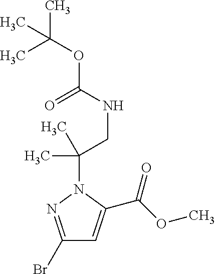 [SMILES: COC(=O)c1cc(Br)nn1C(C)(C)CNC(=O)OC(C)(C)C]

To a solution of methyl 3-bromo-1H-pyrazole-5-carboxylate (3.00 g, 14.6 mmol) and tert-butyl(2-hydroxy-2-methylpropyl) carbamate (3.05 g, 16.1 mmol) in tetrahydrofuran (36 ml) were added triphenylphosphine (7.68 g, 29.3 mmol) and diisopropyl azodicarboxylate (5.92 g, 29.3 mmol) at 20° C. The mixture was stirred at 20° C. for 10 hours. The reaction mixture was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=10:1) to give methyl 3-bromo-1-{1-[(tert-butoxycarbonyl)amino]-2-methylpropan-2-yl}-1H-pyrazole-5-carboxylate (2.80 g, 7.44 mmol, 51% yield) as a white solid.

LC-MS (Method 16): Rₜ=0.645 min; MS (ESIpos): m/z=320.0 [M−56]⁺.

### Intermediate 328

### methyl 1-(1-amino-2-methylpropan-2-yl)-3-bromo-1H-pyrazole-5-carboxylate hydrogen chloride (1/1)

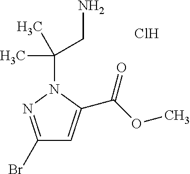

To a solution of methyl 3-bromo-1-{1-[(tert-butoxycarbonyl)amino]-2-methylpropan-2-yl}-1H-pyrazole-5-carboxylate (1.80 g, 4.78 mmol, Intermediate 326) in ethyl acetate (18 ml) was added hydrochloric acid (4 M in ethyl acetate) at 20° C. The mixture was stirred at 20° C. for 2 hours. The reaction mixture was filtered and the solid cake was collected by filtration to give methyl 1-(1-amino-2-methylpropan-2-yl)-3-bromo-1H-pyrazole-5-carboxylate hydrogen chloride (1/1) (1.3 g, 4.16 mmol, 87% yield) as a white solid.

LC-MS (Method 16): Rₜ=0.555 min; MS (ESIpos): m/z=306.3 [(M−36)+H]⁺.

### Intermediate 329

### 2-bromo-7,7-dimethyl-6,7-dihydropyrazolo[1,5-a]pyrazin-4 (5H)-one

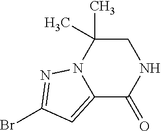

To a solution of methyl 1-(1-amino-2-methylpropan-2-yl)-3-bromo-1H-pyrazole-5-carboxylate hydrochloride (1:1) (1.3 g, 4.16 mmol, Intermediate 328) in methanol (10 ml) was added triethylamine (1.26 g, 12.5 mmol) at 20° C. The mixture was stirred at 50° C. for 12 hours. The reaction mixture was concentrated in vacuo to give 2-bromo-7,7-dimethyl-6,7-dihydropyrazolo[1,5-a]pyrazin-4 (5H)-one (1.00 g, 4.10 mmol, 99% yield) as a white solid.

LC-MS (Method 16): Rₜ=0.503 min; MS (ESIpos): m/z=243.9 [M+H]⁺.

### Intermediate 330

### 2-(4-fluorophenyl)-7,7-dimethyl-6,7-dihydropyrazolo[1,5-a]pyrazin-4 (5H)-one

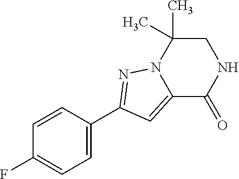 [SMILES: CC1(C)CNC(=O)c2cc(-c3ccc(F)cc3)nn21]

To a solution of 2-bromo-7,7-dimethyl-6,7-dihydropyrazolo[1,5-a]pyrazin-4 (5H)-one (500 mg, 2.05 mmol, Intermediate 329) in 1,4-dioxane (5.0 ml) and water (1.7 ml) were added (4-fluorophenyl) boronic acid (344 mg, 2.46 mmol), [1,1′-bis(diphenylphosphino) ferrocene]palladium (II) dichloride (150 mg, 205 μmol) and sodium carbonate (435 mg, 4.10 mmol) at 20° C. The mixture was stirred under nitrogen atmosphere at 90° C. for 16 hours. The reaction mixture was filtered though a pad of celite and the filtrate was concentrated in vacuo to give a residue. The residue was poured into water and extracted with ethyl acetate. The organic phase was washed with brine, dried over anhydrous sodium sulfate, filtered and concentrated in vacuo. The resulting residue was purified by column chromatography on silica gel (100~200 mesh, 75% ethyl acetate in petroleum ether) to give 2-(4-fluorophenyl)-7,7-dimethyl-6,7-dihydropyrazolo[1,5-a]pyrazin-4 (5H)-one (550 mg, 91% purity, 94% yield) as a yellow solid.

LC-MS (Method 16): Rₜ=0.560 min; MS (ESIpos): m/z=260.3 [M+H]⁺.

### Intermediate 331

### 2-(4-fluorophenyl)-7,7-dimethyl-4,5,6,7-tetrahydropyrazolo[1,5-a]pyrazine

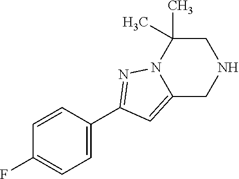 [SMILES: CC1(C)CNCc2cc(-c3ccc(F)cc3)nn21]

To a solution of 2-(4-fluorophenyl)-7,7-dimethyl-6,7-dihydropyrazolo[1,5-a]pyrazin-4 (5H)-one (550 mg, 1.93 mmol, Intermediate 330) in tetrahydrofuran (10 ml) was added borane tetrahydrofuran complex (20 ml, 1 M in tetrahydrofuran) at 0° C. The mixture was stirred at 50° C. for 12 hours. The reaction was quenched with methanol and the resulting solution was concentrated in vacuo to give 2-(4-fluorophenyl)-7,7-dimethyl-4,5,6,7-tetrahydropyrazolo[1,5-a]pyrazine (750 mg, 60% purity, 95% yield) as a pale yellow solid.

LC-MS (Method 16): Rₜ=0.607 min; MS (ESIpos): m/z=246.2 [M+H]⁺.

### Intermediate 332

### tert-butyl 2-(4-fluorophenyl)-7,7-dimethyl-6,7-dihydropyrazolo[1,5-a]pyrazine-5(4H)-carboxylate

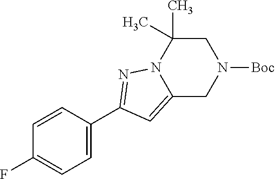 [SMILES: CC(C)(C)OC(=O)N1Cc2cc(-c3ccc(F)cc3)nn2C(C)(C)C1]

To a solution of 2-(4-fluorophenyl)-7,7-dimethyl-4,5,6,7-tetrahydropyrazolo[1,5-a]pyrazine (750 mg, 1.83 mmol, Intermediate 331) in dichloromethane (15 ml) were added di-tert-butyl dicarbonate (630 μl, 2.8 mmol) and triethylamine (510 μl, 3.7 mmol) at 25° C. The mixture was stirred at 25° C. for 16 hours. The mixture was poured into water and extracted with ethyl acetate. The organic phase was washed with brine, dried over anhydrous sodium sulfate, filtered and concentrated in vacuo to give a residue. The residue was purified by column chromatography on silica gel (100~200 mesh, 15% ethyl acetate in petroleum ether) to give tert-butyl 2-(4-fluorophenyl)-7,7-dimethyl-6,7-dihydropyrazolo[1,5-a]pyrazine-5(4H)-carboxylate (600 mg, 95% purity, 90% yield) as a pale yellow solid.

LC-MS (Method 16): Rₜ=0.719 min; MS (ESIpos): m/z=346.4 [M+H]⁺.

### Intermediate 333

### tert-butyl 2-(4-fluorophenyl)-3-iodo-7,7-dimethyl-6,7-dihydropyrazolo[1,5-a]pyrazine-5(4H)-carboxylate

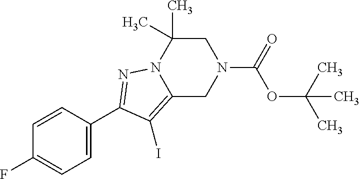 [SMILES: CC(C)(C)OC(=O)N1Cc2c(I)c(-c3ccc(F)cc3)nn2C(C)(C)C1]

To a solution of tert-butyl 2-(4-fluorophenyl)-7,7-dimethyl-6,7-dihydropyrazolo[1,5-a]pyrazine-5(4H)-carboxylate (300 mg, 825 μmol, Intermediate 332) in dichloromethane (10 ml) was added N-iodosuccinimide (371 mg, 1.65 mmol]) at 25° C. The mixture was stirred at 25° C. for 1 hours. The reaction mixture was poured into saturated sodium thiosulfate aqueous solution and extracted with ethyl acetate. The organic phase was washed with brine, dried over anhydrous sodium sulfate, filtered and concentrated in vacuo to give a residue. The residue was purified by column chromatography on silica gel (100~200 mesh, 7% ethyl acetate in petroleum ether) to give tert-butyl 2-(4-fluorophenyl)-3-iodo-7,7-dimethyl-6,7-dihydropyrazolo[1,5-a]pyrazine-5(4H)-carboxylate (740 mg, 97% purity) as a colorless oil.

LC-MS (Method 16): Rₜ=0.769 min; MS (ESIpos): m/z=472.3 [M+H]⁺.

### Intermediate 334

### tert-butyl 2-(4-fluorophenyl)-7,7-dimethyl-3-(pyridin-4-yl)-6,7-dihydropyrazolo[1,5-a]pyrazine-5(4H)-carboxylate

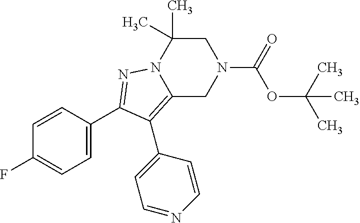 [SMILES: CC(C)(C)OC(=O)N1Cc2c(-c3ccncc3)c(-c3ccc(F)cc3)nn2C(C)(C)C1]

To a solution of tert-butyl 2-(4-fluorophenyl)-3-iodo-7,7-dimethyl-6,7-dihydropyrazolo[1,5-a]pyrazine-5(4H)-carboxylate (740 mg, 1.52 mmol, Intermediate 333) in 1,4-dioxane (9.0 ml) and water (3.0 ml) were added pyridin-4-ylboronic acid (243 mg, 1.98 mmol), sodium carbonate (323 mg, 3.05 mmol) and [1,1′-bis(diphenylphosphino) ferrocene]palladium (II) dichloride dichloromethane complex (144 mg, 152 μmol) at 20° C. The mixture was stirred at 90° C. for 6 hours under nitrogen atmosphere. The reaction mixture was filtered through a pad of celite and the filtrate was concentrated in vacuo to give a residue. The residue was purified by column chromatography on silica gel (100~200 mesh, 47% ethyl acetate in petroleum ether) to give tert-butyl 2-(4-fluorophenyl)-7,7-dimethyl-3-(pyridin-4-yl)-6,7-dihydropyrazolo[1,5-a]pyrazine-5(4H)-carboxylate (540 mg, 95% purity, 80% yield) as a pale yellow solid.

LC-MS (Method 16): Rₜ=0.547 min; MS (ESIpos): m/z=423.4 [M+H]⁺.

### Intermediate 335

### 2-(4-fluorophenyl)-7,7-dimethyl-3-(pyridin-4-yl)-4,5,6,7-tetrahydropyrazolo[1,5-a]pyrazine hydrogen chloride (1/1)

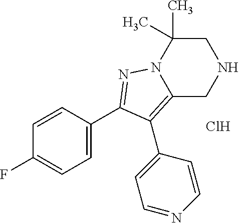 [SMILES: CC1(C)CNCc2c(-c3ccncc3)c(-c3ccc(F)cc3)nn21 ; Cl]

To a solution of tert-butyl 2-(4-fluorophenyl)-7,7-dimethyl-3-(pyridin-4-yl)-6,7-dihydropyrazolo[1,5-a]pyrazine-5(4H)-carboxylate (270 mg, 607 μmol, Intermediate 334) in dioxane (5 ml) was added hydrochloric acid (5 ml, 4 M in dioxane) at 25° C. The mixture was stirred at 25° C. for 1 hours. The reaction mixture was concentrated in vacuo to give 2-(4-fluorophenyl)-7,7-dimethyl-3-(pyridin-4-yl)-4,5,6,7-tetrahydropyrazolo[1,5-a]pyrazine hydrochloride (1:1) (200 mg, 95% purity, 87% yield) as a yellow solid.

LC-MS (Method 16): Rₜ=0.409 min; MS (ESIpos): m/z=323.4 [M+H]⁺.

### Intermediate 336

### tert-butyl 2-(4-chloro-3-methylphenyl)-7,7-dimethyl-3-(pyridin-4-yl)-6,7-dihydropyrazolo[1,5-a]pyrazine-5(4H)-carboxylate

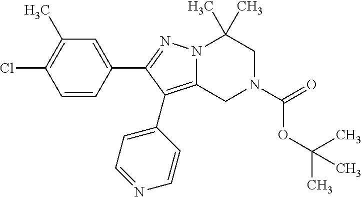 [SMILES: Cc1cc(-c2nn3c(c2-c2ccncc2)CN(C(=O)OC(C)(C)C)CC3(C)C)ccc1Cl]

To a solution of tert-butyl 2-bromo-7,7-dimethyl-3-(pyridin-4-yl)-6,7-dihydropyrazolo[1,5-a]pyrazine-5(4H)-carboxylate (120 mg, 274 μmol, Intermediate 342) in 1,4-dioxane (12 ml) and water (4.0 ml) were added (4-chloro-3-methylphenyl) boronic acid (46.7 mg, 274 μmol), [1,1′-bis(diphenylphosphino) ferrocene]palladium (II) dichloride (20.0 mg, 27.4 μmol) and sodium carbonate (58.1 mg, 548 μmol) at 20° C. The mixture was stirred at 90° C. for 16 hours under nitrogen atmosphere. The reaction mixture was filtered though a pad of celite and the filtrate was concentrated in vacuo to give a residue. The residue was poured into water and extracted with ethyl acetate. The organic phase was washed with brine, dried over anhydrous sodium sulfate, filtered and concentrated in vacuo to give a residue. The residue was purified by column chromatography on silica gel (100~200 mesh, 45% ethyl acetate in petroleum ether) to give the title compound (100 mg, 80% purity, 64% yield) as a yellow oil.

LC-MS (Method 16): Rₜ=0.585 min; MS (ESIpos): m/z=453.1 [M+H]⁺.

### Intermediate 337

### 2-(4-chloro-3-methylphenyl)-7,7-dimethyl-3-(pyridin-4-yl)-4,5,6,7-tetrahydropyrazolo[1,5-a]pyrazine

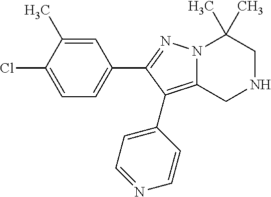 [SMILES: Cc1cc(-c2nn3c(c2-c2ccncc2)CNCC3(C)C)ccc1Cl]

To a solution of tert-butyl 2-(4-chloro-3-methylphenyl)-7,7-dimethyl-3-(pyridin-4-yl)-6,7-dihydropyrazolo[1,5-a]pyrazine-5(4H)-carboxylate (98.0 mg, 173 μmol, Intermediate 336) in ethyl acetate (4 ml) was added hydrochloric acid (2 ml, 4M in ethyl acetate) at 25° C. The mixture was stirred at 25° C. for 1 hour. The reaction mixture was concentrated in vacuo to give 2-(4-chloro-3-methylphenyl)-7,7-dimethyl-3-(pyridin-4-yl)-4,5,6,7-tetrahydropyrazolo[1,5-a]pyrazine (75.0 mg, 80% purity, 98% yield) as a yellow solid.

LC-MS (Method 16): Rₜ=0.463 min; MS (ESIpos): m/z=353.1 [M+H]⁺.

### Intermediate 338

### tert-butyl {2-[3-bromo-5-(hydroxymethyl)-1H-pyrazol-1-yl]-2-methylpropyl}carbamate

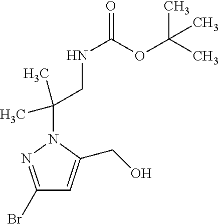 [SMILES: CC(C)(C)OC(=O)NCC(C)(C)n1nc(Br)cc1CO]

To a solution of methyl 3-bromo-1-{1-[(tert-butoxycarbonyl)amino]-2-methylpropan-2-yl}-1H-pyrazole-5-carboxylate (2.80 g, 7.44 mmol, Intermediate 326) in ethanol (56 ml) was added sodium tetrahydroborate (0.845 g, 22.3 mmol) in portions at 0° C. After the addition, the reaction mixture was allowed to warm to 25° C. and stirred for 16 hours. The reaction was quenched with saturated ammonium chloride aqueous solution and the resulting mixture was extracted with ethyl acetate. The organic phase was washed with brine, dried over anhydrous sodium sulfate, filtered and concentrated to give a crude product. The crude product was purified by silica gel column chromatography (petroleum ether:ethyl acetate=10:1 to 1:1) to give tert-butyl {2-[3-bromo-5-(hydroxymethyl)-1H-pyrazol-1-yl]-2-methylpropyl}carbamate (2.50 g, 7.18 mmol, 96% yield) as a yellow oil.

LC-MS (Method 16): Rₜ=0.550 min; MS (ESIpos): m/z=348.0 [M+H]⁺.

### Intermediate 339

### N-[(3-bromo-1-{1-[(tert-butoxycarbonyl)amino]-2-methylpropan-2-yl}-1H-pyrazol-5-yl)methyl]-N,N-diethylethanaminium methanesulfonate

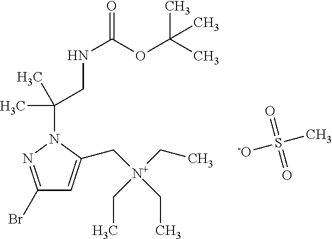

To a solution of tert-butyl {2-[3-bromo-5-(hydroxymethyl)-1H-pyrazol-1-yl]-2-methylpropyl}carbamate (3.00 g, 8.61 mmol, Intermediate 338) and triethylamine (1.74 g, 17.2 mmol) in dichloromethane (38 ml) was added methanesulfonic anhydride (3.00 g, 17.3 mmol) at 0° C. The mixture was stirred at 20° C. for 24 hours. The reaction mixture was concentrated in vacuo to give N-[(3-bromo-1-{1-[(tert-butoxycarbonyl)amino]-2-methylpropan-2-yl}-1H-pyrazol-5-yl)methyl]-N,N-diethylethanaminium methanesulfonate (4.00 g, 7.58 mmol, 88% yield) as a pale yellow solid.

### Intermediate 340

### tert-butyl 2-bromo-7,7-dimethyl-6,7-dihydropyrazolo[1,5-a]pyrazine-5(4H)-carboxylate

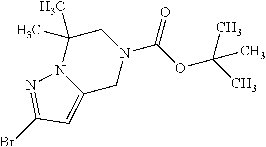 [SMILES: CC(C)(C)OC(=O)N1Cc2cc(Br)nn2C(C)(C)C1]

To a mixture of N-[(3-bromo-1-{1-[(tert-butoxycarbonyl)amino]-2-methylpropan-2-yl}-1H-pyrazol-5-yl)methyl]-N,N-diethylethanaminium methanesulfonate (4.00 g, 7.58 mmol, Intermediate 339) in tetrahydrofuran (28 ml) and N,N-dimethylformamide (57 ml) was added sodium hydride (0.607 g, 15.2 mmol, 60% purity) at 0° C. The mixture was stirred at 20° C. for 16 hours. The reaction mixture was poured into water and extracted with ethyl acetate. The combined organic layers were washed with brine, dried over anhydrous sodium sulfate, filtered and concentrated in vacuo to give tert-butyl 2-bromo-7,7-dimethyl-6,7-dihydropyrazolo[1,5-a]pyrazine-5(4H)-carboxylate (2 g, 6.06 mmol, 80% yield) as a brown oil.

### Intermediate 341

### tert-butyl 2-bromo-3-iodo-7,7-dimethyl-6,7-dihydropyrazolo[1,5-a]pyrazine-5(4H)-carboxylate

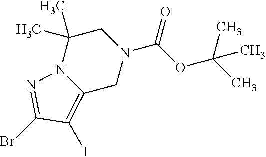 [SMILES: CC(C)(C)OC(=O)N1Cc2c(I)c(Br)nn2C(C)(C)C1]

To a solution of tert-butyl 2-bromo-7,7-dimethyl-6,7-dihydropyrazolo[1,5-a]pyrazine-5(4H)-carboxylate (2.00 g, 6.06 mmol, Intermediate 340) in dichloromethane (10 ml) and methanol (10 ml) was added 1-iodopyrrolidine-2,5-dione (2.04 g, 9.08 mmol) at 20° C. The mixture was stirred at 43° C. for 16 hours. The reaction mixture was concentrated in vacuo to give a residue and the residue was dissolved in ethyl acetate. The resulting solution was poured into saturated sodium sulfite aqueous solution. The organic layers were washed with brine, dried over anhydrous sodium sulfate, filtered and concentrated in vacuo to give a crude product. The crude product was purified by column chromatography on silica gel (petroleum ether:ethyl acetate=10:1) to give tert-butyl 2-bromo-3-iodo-7,7-dimethyl-6,7-dihydropyrazolo[1,5-a]pyrazine-5(4H)-carboxylate (0.800 g, 1.75 mmol, 29% yield) as a white solid.

LC-MS (Method 16): Rₜ=0.718 min; MS (ESIpos): m/z=455.9 [M+H]⁺.

### Intermediate 342

### tert-butyl 2-bromo-7,7-dimethyl-3-(pyridin-4-yl)-6,7-dihydropyrazolo[1,5-a]pyrazine-5(4H)-carboxylate

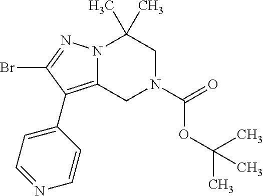 [SMILES: CC(C)(C)OC(=O)N1Cc2c(-c3ccncc3)c(Br)nn2C(C)(C)C1]

To a solution of tert-butyl 2-bromo-3-iodo-7,7-dimethyl-6,7-dihydropyrazolo[1,5-a]pyrazine-5(4H)-carboxylate (400 mg, 877 μmol, Intermediate 341) in 1,4-dioxane (6.0 ml) and water (2.0 ml) were added pyridin-4-ylboronic acid (108 mg, 877 μmol), [1,1′-bis(diphenylphosphino) ferrocene]palladium (II) dichloride (64.2 mg, 87.7 μmol) and sodium carbonate (186 mg, 1.75 mmol) at 20° C. The mixture was stirred at 80° C. for 6 hours under nitrogen atmosphere. The reaction mixture was filtered though a pad of celite and the filtrate was concentrated in vacuo to give a residue. The residue was poured into water and extracted with ethyl acetate. The organic phase was washed with brine, dried over anhydrous sodium sulfate, filtered and concentrated in vacuo to give a residue. The residue was purified by column chromatography on silica gel (100~200 mesh, 30% ethyl acetate in petroleum ether) to give tert-butyl 2-bromo-7,7-dimethyl-3-(pyridin-4-yl)-6,7-dihydropyrazolo[1,5-a]pyrazine-5(4H)-carboxylate (220 mg, 98% purity, 60% yield) as a yellow oil.

LC-MS (Method 16): Rₜ=0.450 min; MS (ESIpos): m/z=407.1 [M+H]⁺.

### Intermediate 343

### tert-butyl 2-(4-chlorophenyl)-7,7-dimethyl-3-(pyridin-4-yl)-6,7-dihydropyrazolo[1,5-a]pyrazine-5(4H)-carboxylate

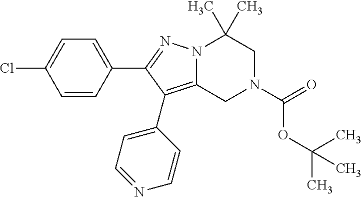 [SMILES: CC(C)(C)OC(=O)N1Cc2c(-c3ccncc3)c(-c3ccc(Cl)cc3)nn2C(C)(C)C1]

To a solution of tert-butyl 2-bromo-7,7-dimethyl-3-(pyridin-4-yl)-6,7-dihydropyrazolo[1,5-a]pyrazine-5(4H)-carboxylate (175 mg, 421 μmol, Intermediate 342) in 1,4-dioxane (3.0 ml) and water (1.0 ml) were added (4-chlorophenyl) boronic acid (65.8 mg, 421 μmol), sodium carbonate (89.3 mg, 842 μmol) and [1,1′-bis(diphenylphosphino) ferrocene]palladium (II) dichloride (30.8 mg, 42.1 μmol) at 25° C. The reaction mixture was stirred at 80° C. for 6 hours under nitrogen atmosphere. The reaction mixture was filtered though a pad of celite and the filtrate was concentrated in vacuo to give a residue. The residue was poured into water and extracted with ethyl acetate. The organic phase was washed with brine, dried over anhydrous sodium sulfate, filtered and concentrated in vacuo to give a residue. The residue was purified by column chromatography on silica gel (100~200 mesh, 45% ethyl acetate in petroleum ether) to give tert-butyl 2-(4-chlorophenyl)-7,7-dimethyl-3-(pyridin-4-yl)-6,7-dihydropyrazolo[1,5-a]pyrazine-5(4H)-carboxylate (150 mg, 78% purity, 63% yield) as a yellow oil.

LC-MS (Method 16): Rₜ=0.582 min; MS (ESIpos): m/z=439.3 [M+H]⁺.

### Intermediate 344

### 2-(4-chlorophenyl)-7,7-dimethyl-3-(pyridin-4-yl)-4,5,6,7-tetrahydropyrazolo[1,5-a]pyrazine-hydrogen chloride (1/1)

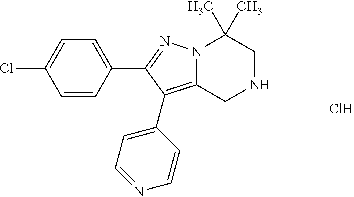 [SMILES: CC1(C)CNCc2c(-c3ccncc3)c(-c3ccc(Cl)cc3)nn21 ; Cl]

To a solution of tert-butyl 2-(4-chlorophenyl)-7,7-dimethyl-3-(pyridin-4-yl)-6,7-dihydropyrazolo[1,5-a]pyrazine-5(4H)-carboxylate (140 mg, 249 μmol, Intermediate 343) in ethyl acetate (5 ml) was added hydrochloric acid (1 ml, 4 M in ethyl acetate) at 25° C. The mixture was stirred at 25° C. for 18 hours. The reaction mixture was concentrated in vacuo to give 2-(4-chlorophenyl)-7,7-dimethyl-3-(pyridin-4-yl)-4,5,6,7-tetrahydropyrazolo[1,5-a]pyrazine hydrochloride (1:1) (115 mg, 78% purity, 96% yield) as a pale yellow solid.

LC-MS (Method 16): Rₜ=0.422 min; MS (ESIpos): m/z=339.1 [M+H]⁺.

### Intermediate 345

### tert-butyl 4,4-dimethyl-2-oxo-1,2lambda⁴, 3-oxathiazolidine-3-carboxylate

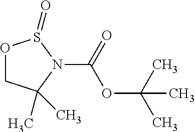

To a solution of thionyl chloride (16 ml, 220 mmol) in acetonitrile (150 ml) was added a solution of tert-butyl (1-hydroxy-2-methylpropan-2-yl) carbamate (17.0 g, 89.8 mmol) in acetonitrile (50 ml) at −40° C. under stirring. After stirring at −40° C. for 5 minutes, pyridine (25 ml, 314 mmol) was added and the resulting mixture was stirred at −40° C. for 0.5 hour before warmed to 0° C. over 0.5 hour. To the reaction mixture was added ethyl acetate. The mixture was washed with hydrogen chloride aqueous solution (0.5 M) and saturated sodium bicarbonate solution, dried over anhydrous sodium sulfate and concentrated under vacuum to give tert-butyl 4,4-dimethyl-2-oxo-1,2lambda⁴,3-oxathiazolidine-3-carboxylate (20.0 g, 85.0 mmol, 95% yield) as a yellow liquid.

¹H NMR (400 MHz, CHLOROFORM-d) δ [ppm]=4.82 (d, J=8.8 Hz, 1H), 4.34 (d, J=8.8 Hz, 1H), 1.60 (s, 3H), 1.52 (s, 9H), 1.41 (s, 3H).

### Intermediate 346

### tert-butyl 4,4-dimethyl-2,2-dioxo-1,2lambda⁶,3-oxathiazolidine-3-carboxylate

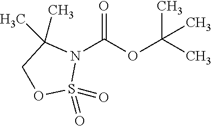 [SMILES: CC(C)(C)OC(=O)N1C(C)(C)COS1(=O)=O]

To a solution of tert-butyl 4,4-dimethyl-2-oxo-1,2lambda⁴,3-oxathiazolidine-3-carboxylate (20.0 g, 85.0 mmol, Intermediate 345) in acetonitrile (100 ml), water (100 ml) and dichloromethane (100 ml) was added sodium periodate (24.3 g, 127 mmol) and ruthenium (III) chloride (5.29 g, 25.5 mol) at 0° C. The mixture was stirred at 25° C. for 1 hour. The reaction mixture was poured into water and extracted with ethyl acetate. The combined extracts were washed with brine, dried over anhydrous sodium sulfate, filtered and concentrated under vacuum to give tert-butyl 4,4-dimethyl-2,2-dioxo-1,2lambda⁶,3-oxathiazolidine-3-carboxylate (21.0 g, 83.6 mmol, 98% yield) as a white solid.

¹H NMR (400 MHz, CHLOROFORM-d) δ [ppm]=4.23 (s, 2H), 1.58 (s, 6H), 1.55 (s, 9H).

### Intermediate 347

### methyl 3-bromo-1-{2-[(tert-butoxycarbonyl)amino]-2-methylpropyl}-1H-pyrazole-5-carboxylate

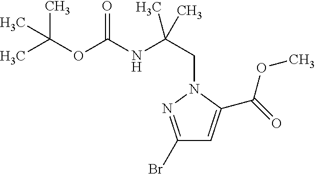 [SMILES: COC(=O)c1cc(Br)nn1CC(C)(C)NC(=O)OC(C)(C)C]

To a solution of methyl 3-bromo-1H-pyrazole-5-carboxylate (13.2 g, 64.3 mmol) and tert-butyl 4,4-dimethyl-2,2-dioxo-1,2lambda⁶,3-oxathiazolidine-3-carboxylate (21.0 g, 83.6 mmol, Intermediate 346) in N,N-dimethylformamide (600 ml) was added cesium carbonate (41.9 g, 129 mmol) at 25° C. and the mixture was stirred at 80° C. for 16 hours. The reaction mixture was poured into water and extracted with ethyl acetate. The combined extracts were concentrated under vacuum to give a residue and the residue was purified by silica gel column chromatography (petroleum ether:ethyl acetate=1:0) to give methyl 3-bromo-1-{2-[(tert-butoxycarbonyl)amino]-2-methylpropyl}-1H-pyrazole-5-carboxylate (14.0 g, 37.2 mmol, 58% yield) as a grey oil.

LC-MS (Method 16): R₁=0.977 min; MS (ESIpos): m/z=276.1 [M−100+H]⁺.

### Intermediate 348

### tert-butyl {1-[3-bromo-5-(hydroxymethyl)-1H-pyrazol-1-yl]-2-methylpropan-2-yl}carbamate

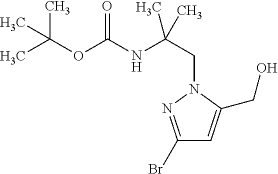 [SMILES: CC(C)(Cn1nc(Br)cc1CO)NC(=O)OC(C)(C)C]

To a solution of methyl 3-bromo-1-{2-[(tert-butoxycarbonyl)amino]-2-methylpropyl}-1H-pyrazole-5-carboxylate (5.00 g, 13.3 mmol, Intermediate 347) in ethanol (80 ml) was added sodium tetrahydroborate (1.52 g, 40.1 mmol) in portions at 0° C. After completion of the addition, the mixture was allowed to warm to 25° C. and stirred for 16 hours. The reaction was quenched with water and then concentrated under vacuum to give a residue. The residue was diluted with water and ethyl acetate. Organic phase was washed with brine and concentrated under vacuum to give tert-butyl {1-[3-bromo-5-(hydroxymethyl)-1H-pyrazol-1-yl]-2-methylpropan-2-yl}carbamate (4.5 g, 12.9 mmol, 97% yield) as a white solid.

LC-MS (Method 16): Rₜ=0.861 min; MS (ESIpos): m/z=348.1 [M+H]⁺.

### Intermediate 349

### N-[(3-bromo-1-{2-[(tert-butoxycarbonyl)amino]-2-methylpropyl}-1H-pyrazol-5-yl)methyl]-N,N-diethylethanaminium methanesulfonate

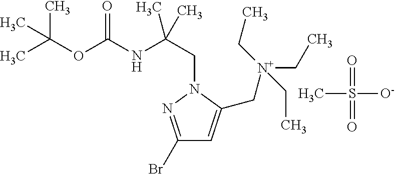

To a solution of tert-butyl {1-[3-bromo-5-(hydroxymethyl)-1H-pyrazol-1-yl]-2-methylpropan-2-yl}carbamate (4.00 g, 11.5 mmol, Intermediate 348) and trimethylamine (9.6 ml, 69.0 mmol) in dichloromethane (80 ml) was added methanesulfonic anhydride (4.00 g, 23.0 mmol) at 0° C. and the mixture was stirred at 25° C. for 24 hours. The reaction mixture was concentrated under vacuum to give N-[(3-bromo-1-{2-[(tert-butoxycarbonyl)amino]-2-methylpropyl}-1H-pyrazol-5-yl)methyl]-N,N-diethylethanaminium methanesulfonate (6.00 g, 11.4 mmol, 99% yield) as a brown oil.

LC-MS (Method 16): Rₜ=0.691 min; MS (ESIpos): m/z=431.2 [M−95]⁺.

### Intermediate 350

### tert-butyl 2-bromo-6,6-dimethyl-6,7-dihydropyrazolo[1,5-a]pyrazine-5(4H)-carboxylate

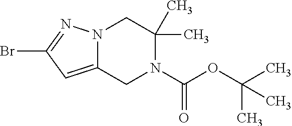 [SMILES: CC(C)(C)OC(=O)N1Cc2cc(Br)nn2CC1(C)C]

To a solution of N-[(3-bromo-1-{2-[(tert-butoxycarbonyl)amino]-2-methylpropyl}-1H-pyrazol-5-yl)methyl]-N,N-diethylethanaminium methanesulfonate (6.00 g, 11.4 mmol, Intermediate 349) in tetrahydrofuran (30 ml) and N,N-dimethylformamide (60 ml) was added sodium hydride (1.82 g, 60% purity, 45.5 mmol) at 25° C. and the mixture was stirred at 25° C. for 16 hours. The reaction was quenched with water and the resulting mixture was extracted with ethyl acetate. The combined extracts were washed brine, dried over anhydrous sodium sulfate and concentrated under vacuum to give tert-butyl 2-bromo-6,6-dimethyl-6,7-dihydropyrazolo[1,5-a]pyrazine-5(4H)-carboxylate (3.60 g, 10.9 mmol, 96% yield) as a yellow solid.

LC-MS (Method 16): Rₜ=0.936 min; MS (ESIpos): m/z=330.1 [M+1]⁺.

### Intermediate 351

### tert-butyl 2-bromo-3-iodo-6,6-dimethyl-6,7-dihydropyrazolo[1,5-a]pyrazine-5(4H)-carboxylate

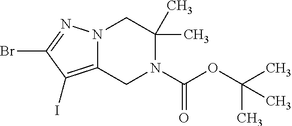 [SMILES: CC(C)(C)OC(=O)N1Cc2c(I)c(Br)nn2CC1(C)C]

To a solution of tert-butyl 2-bromo-6,6-dimethyl-6,7-dihydropyrazolo[1,5-a]pyrazine-5(4H)-carboxylate (3.60 g, 10.9 mmol, Intermediate 350) in dichloromethane (36 ml) and methanol (36 ml) was added N-iodosuccinimide (3.68 g, 16.4 mmol) in portions at 20° C. After completion of the addition, the mixture was stirred at 45° C. for 12 hours. The reaction was quenched with saturated sodium sulfite solution and the resulting mixture was extracted with ethyl acetate. The combined extracts were concentrated under vacuum to give a residue and the residue was purified by silica gel column chromatography (petroleum ether:ethyl acetate=1:0 to 9:1) to give tert-butyl 2-bromo-3-iodo-6,6-dimethyl-6,7-dihydropyrazolo[1,5-a]pyrazine-5(4H)-carboxylate (2.40 g, 5.26 mmol, 48% yield) as a white solid.

LC-MS (Method 16): Rₜ=1.004 min; MS (ESIpos): m/z=456.0 [M+1]⁺.

### Intermediate 352

### tert-butyl 2-bromo-6,6-dimethyl-3-phenyl-6,7-dihydropyrazolo[1,5-a]pyrazine-5(4H)-carboxylate

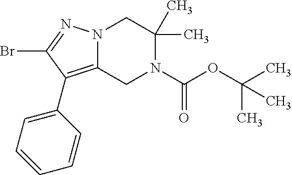 [SMILES: CC(C)(C)OC(=O)N1Cc2c(-c3ccccc3)c(Br)nn2CC1(C)C]

To a solution of tert-butyl 2-bromo-3-iodo-6,6-dimethyl-6,7-dihydropyrazolo[1,5-a]pyrazine-5(4H)-carboxylate (2.20 g, 4.82 mmol, Intermediate 351) and pyridin-4-ylboronic acid (474 mg, 3.86 mmol) in 1,4-dioxane (30 ml) and water (6 ml) were added (1,1′-bis(diphenylphosphino) ferrocene) palladium (II) dichloride (353 mg, 482 μmol) and sodium carbonate (1.02 g, 9.65 mmol) at 20° C. The mixture was degassed with nitrogen and then stirred at 80° C. for 16 hours. The reaction mixture was poured into water and extracted with ethyl acetate. The combined extracts were washed with brine and concentrated under vacuum to give a residue. The residue was purified by silica gel column chromatography (petroleum ether:ethyl acetate=1:0 to 1:1) to give tert-butyl 2-bromo-6,6-dimethyl-3-phenyl-6,7-dihydropyrazolo[1,5-a]pyrazine-5(4H)-carboxylate (1.30 g, 3.20 mmol, 66% yield) as a yellow solid.

LC-MS (Method 16): Rₜ=0.782 min; MS (ESIpos): m/z=407.1 [M+1]⁺

### Intermediate 353

### tert-butyl 2-(4-fluorophenyl)-6,6-dimethyl-3-(pyridin-4-yl)-6,7-dihydropyrazolo[1,5-a]pyrazine-5(4H)-carboxylate

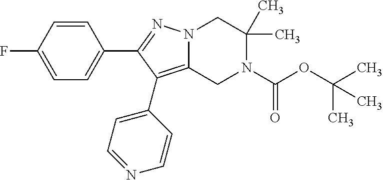 [SMILES: CC(C)(C)OC(=O)N1Cc2c(-c3ccncc3)c(-c3ccc(F)cc3)nn2CC1(C)C]

To a solution of tert-butyl 2-bromo-6,6-dimethyl-3-(pyridin-4-yl)-6,7-dihydropyrazolo[1,5-a]pyrazine-5(4H)-carboxylate (300 mg, 737 μmol, Intermediate 352) and (4-fluorophenyl) boronic acid (206 mg, 1.47 mmol) in 1,4-dioxane (6 ml) and water (1 ml) were added (1,1′-bis(diphenylphosphino) ferrocene) palladium (II) dichloride (53.9 mg, 73.7 μmol) and sodium carbonate (156 mg, 1.47 mmol) at 25° C. The mixture was degassed with nitrogen and stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(4-fluorophenyl)-6,6-dimethyl-3-(pyridin-4-yl)-6,7-dihydropyrazolo[1,5-a]pyrazine-5(4H)-carboxylate (280 mg, 663 μmol, 90% yield) as a yellow oil.

LC-MS (Method 16): Rₜ=0.829 min; MS (ESIpos): m/z=423.3 [M+1]⁺

### Intermediate 354

### 2-(4-fluorophenyl)-6,6-dimethyl-3-(pyridin-4-yl)-4,5,6,7-tetrahydropyrazolo[1,5-a]pyrazine hydrogen chloride (1:1)

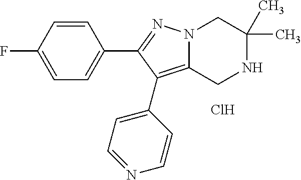 [SMILES: CC1(C)Cn2nc(-c3ccc(F)cc3)c(-c3ccncc3)c2CN1 ; Cl]

To a solution of tert-butyl 2-(4-fluorophenyl)-6,6-dimethyl-3-(pyridin-4-yl)-6,7-dihydropyrazolo[1,5-a]pyrazine-5(4H)-carboxylate (280 mg, 663 μmol, Intermediate 353) in ethyl aceate (1 ml) was added hydrochloric acid (4.0 M in ethyl acetate, 14 ml, 56.0 mmol) at 25° C. and the mixture was stirred at 25° C. for 1 hour. The reaction mixture was concentrated under vacuum to give 2-(4-fluorophenyl)-6,6-dimethyl-3-(pyridin-4-yl)-4,5,6,7-tetrahydropyrazolo[1,5-a]pyrazine hydrogen chloride (1:1) (220 mg, 613 μmol, 93% yield) as a brown solid.

LC-MS (Method 20): Rₜ=0.863 min; MS (ESIpos): m/z=323.1 [M+1]⁺

### Intermediate 355

### tert-butyl 2-(4-chlorophenyl)-6,6-dimethyl-3-(pyridin-4-yl)-6,7-dihydropyrazolo[1,5-a]pyrazine-5(4H)-carboxylate

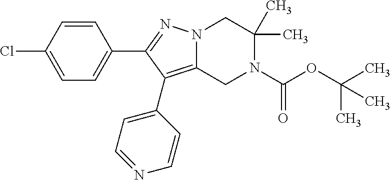 [SMILES: CC(C)(C)OC(=O)N1Cc2c(-c3ccncc3)c(-c3ccc(Cl)cc3)nn2CC1(C)C]

To a solution of tert-butyl 2-bromo-6,6-dimethyl-3-(pyridin-4-yl)-6,7-dihydropyrazolo[1,5-a]pyrazine-5(4H)-carboxylate (300 mg, 737 μmol, Intermediate 353) and (4-chlorophenyl) boronic acid (346 mg, 2.21 mmol) in water (1 ml) and 1,4-dioxane (5 ml) were added (1,1′-bis(diphenylphosphino) ferrocene) palladium (II) dichloride (53.9 mg, 73.7 μmol) and sodium carbonate (156 mg, 1.47 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(4-chlorophenyl)-6,6-dimethyl-3-(pyridin-4-yl)-6,7-dihydropyrazolo[1,5-a]pyrazine-5(4H)-carboxylate (290 mg, 661 μmol, 90% yield) as a yellow oil.

LC-MS (Method 16): Rₜ=0.877 min; MS (ESIpos): m/z=439.2 [M+1]⁺

### Intermediate 356

### 2-(4-chlorophenyl)-6,6-dimethyl-3-(pyridin-4-yl)-4,5,6,7-tetrahydropyrazolo[1,5-a]pyrazine hydrogen chloride (1:1)

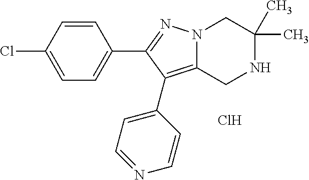 [SMILES: CC1(C)Cn2nc(-c3ccc(Cl)cc3)c(-c3ccncc3)c2CN1 ; Cl]

To a solution of tert-butyl 2-(4-chlorophenyl)-6,6-dimethyl-3-(pyridin-4-yl)-6,7-dihydropyrazolo[1,5-a]pyrazine-5(4H)-carboxylate (290 mg, 661 μmol, Intermediate 355) in ethyl aceate (1 ml) was added hydrochloric acid (4.0 M in ethyl acetate, 10 ml, 40.0 mmol) at 25° C. and the mixture was stirred at 25° C. for 1 hour. The precipitate was collected by filtration to give 2-(4-chlorophenyl)-6,6-dimethyl-3-(pyridin-4-yl)-4,5,6,7-tetrahydropyrazolo[1,5-a]pyrazine hydrogen chloride (1:1) (220 mg, 586 μmol, 89% yield) as a white solid.

LC-MS (Method 20): Rₜ=0.893 min; MS (ESIpos): m/z=339.1 [M+1]⁺

### Intermediate 357

### tert-butyl 2-(4-chloro-3-methylphenyl)-6,6-dimethyl-3-(pyridin-4-yl)-6,7-dihydropyrazolo[1,5-a]pyrazine-5(4H)-carboxylate

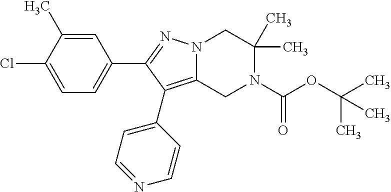 [SMILES: Cc1cc(-c2nn3c(c2-c2ccncc2)CN(C(=O)OC(C)(C)C)C(C)(C)C3)ccc1Cl]

To a solution of tert-butyl 2-bromo-6,6-dimethyl-3-(pyridin-4-yl)-6,7-dihydropyrazolo[1,5-a]pyrazine-5(4H)-carboxylate (300 mg, 737 μmol, Intermediate 353) and (4-chloro-3-methylphenyl) boronic acid (377 mg, 2.21 mmol) in water (1 ml) and 1,4-dioxane (5 ml) were added (1,1′-bis(diphenylphosphino) ferrocene) palladium (II) dichloride (53.9 mg, 73.7 μmol) and sodium carbonate (156 mg, 1.47 mmol) at 25° C. The mixture was degassed with nitrogen and then stirred at 90° C. for 16 hours. The reaction mixture was concentrated under vacuum to give a residue and the residue was purified by silica gel column chromatography (petroleum ether:ethyl acetate=1:0 to 1:1) to give tert-butyl 2-(4-chloro-3-methylphenyl)-6,6-dimethyl-3-(pyridin-4-yl)-6,7-dihydropyrazolo[1,5-a]pyrazine-5(4H)-carboxylate (300 mg, 662 μmol, 90% yield) as a yellow oil.

LC-MS (Method 16): Rₜ=0.900 min; MS (ESIpos): m/z=453.2 [M+1]⁺

### Intermediate 358

### 2-(4-chloro-3-methylphenyl)-6,6-dimethyl-3-(pyridin-4-yl)-4,5,6,7-tetrahydropyrazolo[1,5-a]pyrazine hydrogen chloride (1:1)

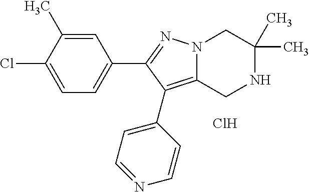 [SMILES: Cc1cc(-c2nn3c(c2-c2ccncc2)CNC(C)(C)C3)ccc1Cl ; Cl]

To a solution of tert-butyl 2-(4-chloro-3-methylphenyl)-6,6-dimethyl-3-(pyridin-4-yl)-6,7-dihydropyrazolo[1,5-a]pyrazine-5(4H)-carboxylate (300 mg, 662 μmol, Intermediate 357) in ethyl aceate (1 ml) was added hydrochloric acid (4.0 M in ethyl acetate, 10 ml, 40.0 mmol) at 25° C. and the reaction mixture was stirred at 25° C. for 1 hour. The precipitate was collected by filtration to give 2-(4-chloro-3-methylphenyl)-6,6-dimethyl-3-(pyridin-4-yl)-4,5,6,7-tetrahydropyrazolo[1,5-a]pyrazine hydrogen chloride (1:1) (230 mg, 590 μmol, 89% yield) as a white solid.

LC-MS (Method 20): Rₜ=0.921 min; MS (ESIpos): m/z=353.1 [M+1]⁺

### Intermediate 359

### (2RS)-1-amino-4-(benzyloxy) butan-2-ol

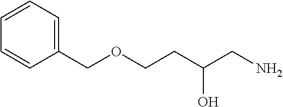 [SMILES: NCC(O)CCOCc1ccccc1]

The solution of (2RS)-2-[2-(benzyloxy)ethyl]oxirane (10.0 g, 56.1 mmol) in ammonium hydroxide (500 ml, 28-30 wt. % solution in water) and methanol (20 ml) was stirred at 25° C. for 6 hours. The reaction mixture was extracted with ethyl acetate. The combined organic phase was dried over anhydrous sodium sulfate, filtered and concentrated under reduced pressure to give (2RS)-1-amino-4-(benzyloxy) butan-2-ol (10.0 g, 75% purity, 68% yield) as a colorless oil.

LC-MS (Method 16): Rₜ=0.253 min; MS (ESIpos): m/z=196.1 [M+H]⁺.

### Intermediate 360

### tert-butyl [(2RS)-4-(benzyloxy)-2-hydroxybutyl]carbamate

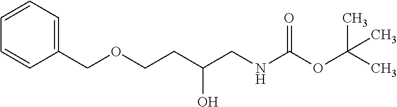 [SMILES: CC(C)(C)OC(=O)NCC(O)CCOCc1ccccc1]

To a solution of (2RS)-1-amino-4-(benzyloxy) butan-2-ol (10.0 g, 75% purity, 38.4 mmol, Intermediate 359) and di-tert-butyl dicarbonate (8.8 ml, 38 mmol) in dichloromethane (75 ml) was added triethylamine (5.4 ml, 38.0 mmol) at 25° C. The mixture was stirred at 25° C. for 1 hour. The reaction mixture was concentrated under reduced pressure to give a residue.

The residue was purified by column chromatography on silica gel (100~200 mesh, petroleum ether:ethyl acetate=10:1 to 3:1) to give tert-butyl [(2RS)-4-(benzyloxy)-2-hydroxybutyl]carbamate (11.6 g, 72% purity, 74% yield) as a colorless oil.

LC-MS (Method 16): Rₜ=0.848 min; MS (ESIpos): m/z=196.1 [M+H]⁺

### Intermediate 361

### ethyl 1-{(2RS)-4-(benzyloxy)-1-[(tert-butoxycarbonyl)amino]butan-2-yl}-3-bromo-1H-pyrazole-5-carboxylate

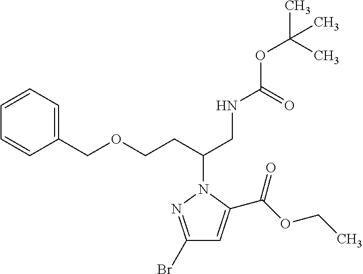 [SMILES: CCOC(=O)c1cc(Br)nn1C(CCOCc1ccccc1)CNC(=O)OC(C)(C)C]

To a solution of tert-butyl [(2RS)-4-(benzyloxy)-2-hydroxybutyl]carbamate (12.6 g, 72% purity, 30.7 mmol, Intermediate 360), ethyl 5-bromo-1H-pyrazole-3-carboxylate (6.73 g, 30.7 mmol) and triphenylphosphine (16.1 g, 61.4 mmol) in tetrahydrofuran (120 ml) was added dropwise diethyl azodicarboxylate (9.7 ml, 61 mmol) at 0° C. The mixture was stirred at 25° C. for 2 hours. The reaction mixture was concentrated under reduced pressure to give a residue. The residue was purified by column chromatography on silica gel (100~200 mesh, petroleum ether:ethyl acetate=10:1 to 1:1) to give ethyl 1-{(2RS)-4-(benzyloxy)-1-[(tert-butoxycarbonyl)amino]butan-2-yl}-3-bromo-1H-pyrazole-5-carboxylate (15.7 g, 83% purity, 85% yield) as a colorless oil.

LC-MS (Method 16): Rₜ=1.043 min; MS (ESIpos): m/z=396.0 [M+H]⁺.

¹H NMR (400 MHz, DMSO-d₆) δ [ppm]=7.36-7.24 (m, 6H), 6.79 (s, 1H), 5.78-5.55 (m, 1H), 4.98-4.65 (m, 1H), 4.38 (d, J=4.8 Hz, 2H), 4.26 (t, J=7.2 Hz, 1H), 3.63-3.53 (m, 2H), 3.49-3.41 (m, 1H), 3.27-3.16 (m, 1H), 2.34-2.21 (m, 1H), 2.1 (dd, J=13.2, 7.2 Hz, 1H), 1.41 (s, 9H), 1.32 (t, J=7.2 Hz, 3H).

### Intermediate 362

### (7RS)-7-[2-(benzyloxy)ethyl]-2-bromo-6,7-dihydropyrazolo[1,5-a]pyrazin-4 (5H)-one

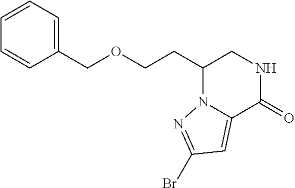 [SMILES: O=C1NCC(CCOCc2ccccc2)n2nc(Br)cc21]

To a solution of ethyl 1-{(2RS)-4-(benzyloxy)-1-[(tert-butoxycarbonyl)amino]butan-2-yl}-3-bromo-1H-pyrazole-5-carboxylate (16.0 g, 83% purity, 26.8 mmol, Intermediate 361) in dichloromethane (150 ml) was added trifluoroacetic acid (50 ml, 650 mmol) at 25° C. The mixture was stirred at 25° C. for 2 hours. The mixture was concentrated under reduced pressure to give a residue. The residue was dissolved in 1,4-dioxane (30 ml) and pH of the resulting solution was adjusted to 9 with saturated sodium bicarbonate aqueous solution. The resulting mixture was stirred at 25° C. for 16 hours. The reaction mixture was extracted with ethyl acetate and the organic phase was washed with brine, dried over anhydrous sodium sulfate, filtered and concentrated under reduced pressure to give (7RS)-7-[2-(benzyloxy)ethyl]-2-bromo-6,7-dihydropyrazolo[1,5-a]pyrazin-4 (5H)-one (11.7 g, 62% purity, 77% yield) as a white solid.

LC-MS (Method 16): Rₜ=0.861 min; MS (ESIpos): m/z=352.0 [M+H]⁺

¹H NMR (400 MHz, DMSO-d₆) δ [ppm]=7.33-7.15 (m, 6H), 6.77 (s, 1H), 4.58-4.50 (m, 1H), 4.50-4.40 (m, 2H), 3.84-3.75 (m, 1H), 3.61-3.48 (m, 3H), 2.38-2.26 (m, 1H), 2.07-1.98 (m, 1H)

### Intermediate 363

### (7RS)-7-[2-(benzyloxy)ethyl]-2-(4-fluorophenyl)-6,7-dihydropyrazolo[1,5-a]pyrazin-4 (5H)-one

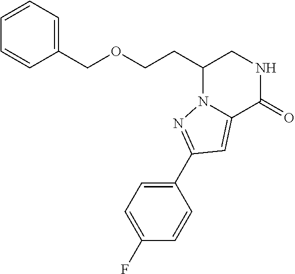 [SMILES: O=C1NCC(CCOCc2ccccc2)n2nc(-c3ccc(F)cc3)cc21]

To a solution of (7S)-7-[2-(benzyloxy)ethyl]-2-bromo-6,7-dihydropyrazolo[1,5-a]pyrazin-4 (5H)-one (3.10 g, 8.85 mmol, Intermediate 362) and (4-fluorophenyl) boronic acid (1.36 g, 9.74 mmol) in 1,4-dioxane (31 ml) and water (9.3 ml) were added sodium carbonate (3.06 g, 22.1 mmol) and dichloro[1,1-bis(diphenylphosphino) ferrocene]palladium (II) (648 mg, 885 μmol) at 25° C. The mixture was stirred at 100° C. for 16 hours under nitrogen atmosphere. The reaction mixture was concentrated under reduced pressure to give a residue. The residue was purified by column chromatography on silica gel (100~200 mesh, petroleum ether:ethyl acetate=10:1 to 1:1) to give (7RS)-7-[2-(benzyloxy)ethyl]-2-(4-fluorophenyl)-6,7-dihydropyrazolo[1,5-a]pyrazin-4 (5H)-one (3.00 g, 90% purity, 83% yield) as a colorless oil.

LC-MS (Method 16): Rₜ=0.910 min; MS (ESIpos): m/z=366.1 [M+H]⁺.

¹H NMR (400 MHz, DMSO-d6) δ [ppm]=7.83-7.74 (m, 2H), 7.40-7.35 (m, 4H), 7.33 (dd, J=5.2, 3.6 Hz, 1H), 7.15-7.08 (m, 3H), 6.24 (s, 1H), 4.73-4.65 (m, 1H), 4.61-4.51 (m, 2H), 4.00-3.90 (m, 1H), 3.75-3.63 (m, 3H), 2.51-2.40 (m, 1H), 2.22-2.11 (m, 1H).

### Intermediate 364

### (7RS)-7-[2-(benzyloxy)ethyl]-2-(4-fluorophenyl)-4,5,6,7-tetrahydropyrazolo[1,5-a]pyrazine

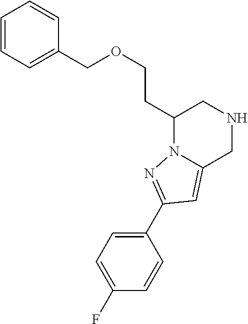 [SMILES: Fc1ccc(-c2cc3n(n2)C(CCOCc2ccccc2)CNC3)cc1]

To a solution of (7RS)-7-[2-(benzyloxy)ethyl]-2-(4-fluorophenyl)-6,7-dihydropyrazolo[1,5-a]pyrazin-4 (5H)-one (3.00 g, 90% purity, 7.39 mmol, Intermediate 363) in tetrahydrofuran (50 ml) was added lithium aluminum hydride (1.36 g, 36.9 mmol) at 0° C. The mixture was stirred at 60° C. for 16 hours. The reaction was quenched with 15% of sodium hydroxide aqueous solution. The resulting mixture was filtered and washed with methanol. The filtrate was concentrated under reduced pressure to give (7RS)-7-[2-(benzyloxy)ethyl]-2-(4-fluorophenyl)-4,5,6,7-tetrahydropyrazolo[1,5-a]pyrazine (3.20 g, 70% purity, 86% yield) as a white solid.

LC-MS (Method 16): Rₜ=0.794 min; MS (ESIpos): m/z=352.1 [M+H]⁺.

### Intermediate 365

### tert-butyl (7RS)-7-[2-(benzyloxy)ethyl]-2-(4-fluorophenyl)-6,7-dihydropyrazolo[1,5-a]pyrazine-5(4H)-carboxylate

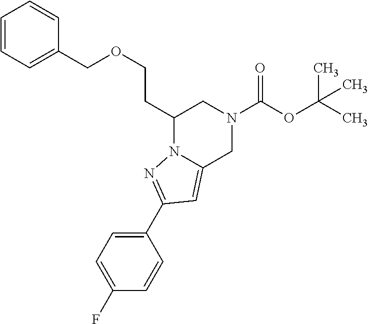 [SMILES: CC(C)(C)OC(=O)N1Cc2cc(-c3ccc(F)cc3)nn2C(CCOCc2ccccc2)C1]

To a solution of (7RS)-7-[2-(benzyloxy)ethyl]-2-(4-fluorophenyl)-4,5,6,7-tetrahydropyrazolo[1,5-a]pyrazine (4.25 g, 12.1 mmol, Intermediate 364) in dichloromethane (50 ml) were added di-tert-butyl dicarbonate (2.8 ml, 12 mmol) and triethylamine (1.7 ml, 12 mmol) at 25° C. The mixture was stirred at 25° C. for 6 hours. The mixture was concentrated under reduced pressure to give a residue. The residue was purified by column chromatography on silica gel (100~200 mesh, petroleum ether:ethyl acetate=10:1 to 3:1) to give tert-butyl (7RS)-7-[2-(benzyloxy)ethyl]-2-(4-fluorophenyl)-6,7-dihydropyrazolo[1,5-a]pyrazine-5(4H)-carboxylate (4.00 g, 94% purity, 69% yield) as a light yellow oil. 10

LC-MS (Method 16): Rₜ=1.070 min; MS (ESIpos): m/z=452.2 [M+H]⁺.

### Intermediate 366

### tert-butyl (7RS)-7-[2-(benzyloxy)ethyl]-2-(4-fluorophenyl)-3-iodo-6,7-dihydropyrazolo[1,5-a]pyrazine-5(4H)-carboxylate

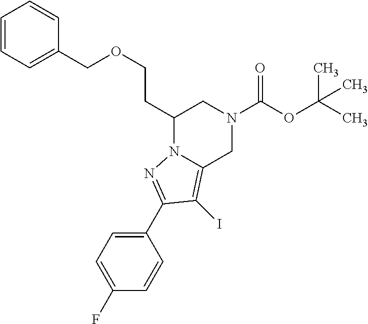 [SMILES: CC(C)(C)OC(=O)N1Cc2c(I)c(-c3ccc(F)cc3)nn2C(CCOCc2ccccc2)C1]

To a solution of tert-butyl (7RS)-7-[2-(benzyloxy)ethyl]-2-(4-fluorophenyl)-6,7-dihydropyrazolo[1,5-a]pyrazine-5(4H)-carboxylate (1.60 g, 59% purity, 2.09 mmol, Intermediate 365) in dichloromethane (20 ml) and methanol (4.0 ml) was added N-iodosuccinimide (706 mg, 3.14 mmol) at 25° C. The mixture was stirred at 40° C. for 16 hours. The reaction was quenched with saturated sodium thiosulfate aqueous solution. The resulting mixture was extracted with dichloromethane and the combined organic phase was washed with brine, dried over anhydrous sodium sulfate, filtered and concentrated under reduced pressure to give a residue. The residue was purified by column chromatography on silica gel (100~200 mesh, petroleum ether:ethyl acetate=10:1 to 3:1) to give tert-butyl (7RS)-7-[2-(benzyloxy)ethyl]-2-(4-fluorophenyl)-3-iodo-6,7-dihydropyrazolo[1,5-a]pyrazine-5(4H)-carboxylate (1.10 g, 95% purity, 87% yield) as a yellow oil.

LC-MS (Method 16): Rₜ=1.131 min; MS (ESIpos): m/z=578.1 [M+H]⁺.

¹H NMR (400 MHz, DMSO-d6) δ [ppm]=7.85-7.77 (m, 2H), 7.39-7.28 (m, 5H), 7.12 (t, J=8.8 Hz, 2H), 4.65-4.50 (m, 3H), 4.49-4.33 (m, 2H), 4.04 (dd, J=14.0, 4.4 Hz, 1H), 3.81-3.67 (m, 3H), 2.38 (dd, J=14.0, 6.4 Hz, 1H), 2.01 (dd, J=14.0, 7.6 Hz, 1H), 1.51 (s, 9H).

### Intermediate 367

### tert-butyl (7RS)-7-[2-(benzyloxy)ethyl]-2-(4-fluorophenyl)-3-(pyridin-4-yl)-6,7-dihydropyrazolo[1,5-a]pyrazine-5(4H)-carboxylate

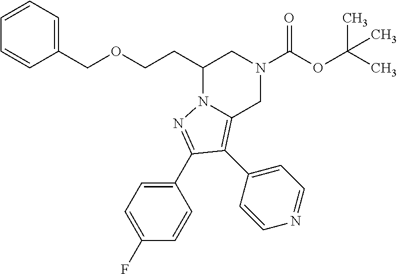 [SMILES: CC(C)(C)OC(=O)N1Cc2c(-c3ccncc3)c(-c3ccc(F)cc3)nn2C(CCOCc2ccccc2)C1]

To a solution of tert-butyl (7RS)-7-[2-(benzyloxy)ethyl]-2-(4-fluorophenyl)-3-iodo-6,7-dihydropyrazolo[1,5-a]pyrazine-5(4H)-carboxylate (4.60 g, 95% purity, 7.57 mmol, Intermediate 366) and pyridin-4-ylboronic acid (1.02 g, 8.32 mmol) in 1,4-dioxane (50 ml) and water (5.0 ml) were added sodium carbonate (2.61 g, 18.9 mmol) and dichloro[1,1-bis(diphenylphosphino) ferrocene]palladium (II) (554 mg, 757 μmol) at 25° C. The mixture was stirred at 100° C. for 16 hours under nitrogen atmosphere. The residue was concentrated under reduced pressure to give a residue. The residue was purified by column chromatography on silica gel (100~200 mesh, petroleum ether:ethyl acetate=10:1 to 1:1) to give tert-butyl (7RS)-7-[2-(benzyloxy)ethyl]-2-(4-fluorophenyl)-3-(pyridin-4-yl)-6,7-dihydropyrazolo[1,5-a]pyrazine-5(4H)-carboxylate (4.30 g, 98% purity, 105% yield) as a yellow oil.

LC-MS (Method 16): Rₜ=0.881 min; MS (ESIpos): m/z=529.3 [M+H]⁺.

### Intermediate 368

### tert-butyl (7RS)-2-(4-fluorophenyl)-7-(2-hydroxyethyl)-3-(pyridin-4-yl)-6,7-dihydropyrazolo[1,5-a]pyrazine-5(4H)-carboxylate

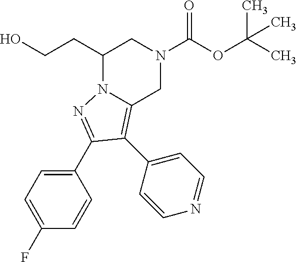 [SMILES: CC(C)(C)OC(=O)N1Cc2c(-c3ccncc3)c(-c3ccc(F)cc3)nn2C(CCO)C1]

To a solution of tert-butyl (7RS)-7-[2-(benzyloxy)ethyl]-2-(4-fluorophenyl)-3-(pyridin-4-yl)-6,7-dihydropyrazolo[1,5-a]pyrazine-5(4H)-carboxylate (1.00 g, 98% purity, 1.85 mmol, Intermediate 367) in methanol (20 ml) was added palladium on carbon (395 mg, 10% purity, 371 μmol) and palladium (II) hydroxide (104 mg, 50% purity, 371 μmol) at 25° C. The suspension was degassed with hydrogen and stirred at 60° C. for 72 hours. The mixture was filtered through a pad of celite and washed with methanol. The filtrate was concentrated under reduced pressure to give tert-butyl (7RS)-2-(4-fluorophenyl)-7-(2-hydroxyethyl)-3-(pyridin-4-yl)-6,7-dihydropyrazolo[1,5-a]pyrazine-5(4H)-carboxylate (570 mg, 94% purity, 66% yield) as a yellow solid.

LC-MS (Method 16): Rₜ=0.765 min; MS (ESIpos): m/z=493.2 [M+H]⁺

### Intermediate 369

### 2-[(7RS)-2-(4-fluorophenyl)-3-(pyridin-4-yl)-4,5,6,7-tetrahydropyrazolo[1,5-a]pyrazin-7-yl]ethan-1-ol hydrogen chloride (1/1)

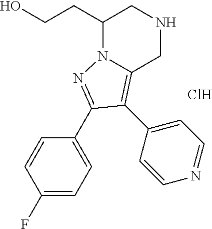 [SMILES: OCCC1CNCc2c(-c3ccncc3)c(-c3ccc(F)cc3)nn21 ; Cl]

To a solution of tert-butyl (7RS)-2-(4-fluorophenyl)-7-(2-hydroxyethyl)-3-(pyridin-4-yl)-6,7-dihydropyrazolo[1,5-a]pyrazine-5(4H)-carboxylate (300 mg, 94% purity, 643 μmol Intermediate 368) in ethyl acetate (30 ml) was added hydrochloric acid (30 ml, 4 M in ethyl acetate) at 25° C. The mixture was stirred at 25° C. for 3 hours. The reaction mixture was concentrated under reduced pressure to give 2-[(7RS)-2-(4-fluorophenyl)-3-(pyridin-4-yl)-4,5,6,7-tetrahydropyrazolo[1,5-a]pyrazin-7-yl]ethanol hydrochloride (1:1) (348 mg, 67% purity, 96% yield) as a colorless oil.

LC-MS (Method 16): Rₜ=0.645 min; MS (ESIpos): m/z=339.1 [M+H]⁺

### Intermediate 370

### tert-butyl (7RS)-2-(4-fluorophenyl)-7-(2-oxoethyl)-3-(pyridin-4-yl)-6,7-dihydropyrazolo[1,5-a]pyrazine-5(4H)-carboxylate

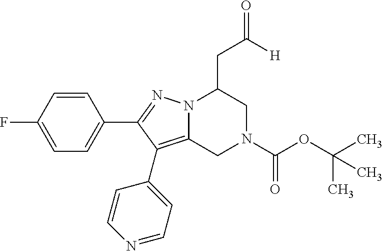 [SMILES: CC(C)(C)OC(=O)N1Cc2c(-c3ccncc3)c(-c3ccc(F)cc3)nn2C(CC=O)C1]

To a mixture of tert-butyl (7RS)-2-(4-fluorophenyl)-7-(2-hydroxyethyl)-3-(pyridin-4-yl)-6,7-dihydropyrazolo[1,5-a]pyrazine-5(4H)-carboxylate (564 mg, 88% purity, 1.13 mmol, Intermediate 368) in dichloromethane (8.0 ml) was added 1,1,1-triacetoxy-1lambda⁵,2-benziodoxol-3 (1H)-one (1.01 g, 2.38 mmol) in one portion at 25° C. The mixture was stirred at 25° C. for 1 hours. The reaction was quenched with a solution of sodium thiosulfate and the resulting mixture was extracted with ethyl acetate. The combined organic phase was washed with brine, dried over anhydrous sodium sulfate, filtered and concentrated under vacuum to give tert-butyl (RS)-2-(4-fluorophenyl)-7-(2-oxoethyl)-3-(pyridin-4-yl)-6,7-dihydropyrazolo[1,5-a]pyrazine-5(4H)-carboxylate (1.00 g, 37% purity, 75% yield) as a brown oil.

LC-MS (Method 20): Rₜ=0.978 min; MS (ESIpos): m/z=437.1 [M+H]⁺

### Intermediate 371

### [(7RS)-5-(tert-butoxycarbonyl)-2-(4-fluorophenyl)-3-(pyridin-4-yl)-4,5,6,7-tetrahydropyrazolo[1,5-a]pyrazin-7-yl]acetic acid

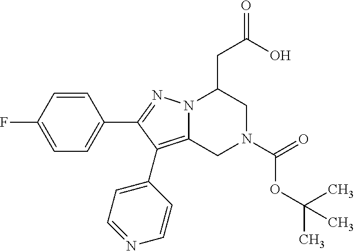 [SMILES: CC(C)(C)OC(=O)N1Cc2c(-c3ccncc3)c(-c3ccc(F)cc3)nn2C(CC(=O)O)C1]

To a solution of tert-butyl (7RS)-2-(4-fluorophenyl)-7-(2-oxoethyl)-3-(pyridin-4-yl)-6,7-dihydropyrazolo[1,5-a]pyrazine-5(4H)-carboxylate (1.00 g, 37% purity, 848 μmol, Intermediate 370 in tert-butanol (6.0 ml) were added a solution of sodium chlorite (107 mg, 1.19 mmol) in water (3.0 ml) and sodium dihydrogen phosphate (101 mg, 1.19 mmol) in portions at 0° C. The mixture was stirred at 25° C. for 1 hour. The reaction was quenched with a solution of sodium thiosulfate and the resulting mixture was extracted with ethyl acetate. The combined organic phase was washed with brine, dried over anhydrous sodium sulfate, filtered and concentrated under vacuum to give [(7RS)-5-(tert-butoxycarbonyl)-2-(4-fluorophenyl)-3-(pyridin-4-yl)-4,5,6,7-tetrahydropyrazolo[1,5-a]pyrazin-7-yl]acetic acid (60.0 mg, 62% purity, 10% yield; 230 mg, 81% purity, 49% yield) as a brown oil.

LC-MS (Method 20): Rₜ=0.716 min; MS (ESIpos): m/z=453.1 [M+H]⁺.

### Intermediate 372

### tert-butyl (7RS)-7-(2-amino-2-oxoethyl)-2-(4-fluorophenyl)-3-(pyridin-4-yl)-6,7-dihydropyrazolo[1,5-a]pyrazine-5(4H)-carboxylate

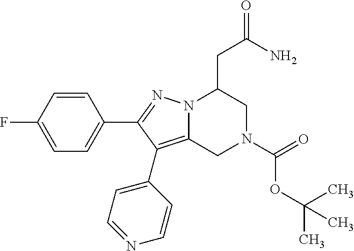 [SMILES: CC(C)(C)OC(=O)N1Cc2c(-c3ccncc3)c(-c3ccc(F)cc3)nn2C(CC(N)=O)C1]

To a solution of [(7RS)-5-(tert-butoxycarbonyl)-2-(4-fluorophenyl)-3-(pyridin-4-yl)-4,5,6,7-tetrahydropyrazolo[1,5-a]pyrazin-7-yl]acetic acid (80.0 mg, 81% purity, 143 μmol, Intermediate 371) and ammonium chloride (15.3 mg, 286 μmol) in N,N-dimethylformamide (2.0 ml) were added O-(7-azabenzotriazol-1-yl)-N,N,N′,N′-tetramethyluronium hexafluorophosphate (59.9 mg, 158 μmol) and N,N-diisopropylethylamine (50 μl, 290 μmol) at 25° C. The mixture was stirred at 25° C. for 16 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by preparative TLC (petroleum ether:ethyl acetate=0:1) to give tert-butyl (RS)-7-(2-amino-2-oxoethyl)-2-(4-fluorophenyl)-3-(pyridin-4-yl)-6,7-dihydropyrazolo[1,5-a]pyrazine-5(4H)-carboxylate (50.0 mg, 93% purity, 72% yield) as a colorless oil.

LC-MS (Method 16): Rₜ=0.723 min; MS (ESIpos): m/z=452.1 [M+H]⁺

### Intermediate 373

### 2-[(7RS)-2-(4-fluorophenyl)-3-(pyridin-4-yl)-4,5,6,7-tetrahydropyrazolo[1,5-a]pyrazin-7-yl]acetamide-hydrogen chloride (1/1)

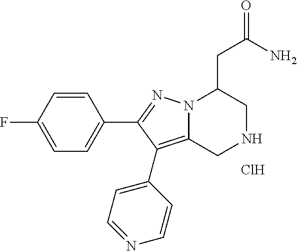 [SMILES: NC(=O)CC1CNCc2c(-c3ccncc3)c(-c3ccc(F)cc3)nn21 ; Cl]

The solution of tert-butyl (7RS)-7-(2-amino-2-oxoethyl)-2-(4-fluorophenyl)-3-(pyridin-4-yl)-6,7-dihydropyrazolo[1,5-a]pyrazine-5(4H)-carboxylate (50.0 mg, 93% purity, 103 μmol, Intermediate 372) in hydrochloric acid (1.5 ml, 4M in 1,4-dioxane) was stirred at 25° C. for 3 hours. The reaction mixture was concentrated under reduced pressure to give 2-[(7RS)-2-(4-fluorophenyl)-3-(pyridin-4-yl)-4,5,6,7-tetrahydropyrazolo[1,5-a]pyrazin-7-yl]acetamide hydrogen chloride (1/1) (35.0 mg, 53% purity, 46% yield) as a colorless oil.

LC-MS (Method 14): Rₜ=0.121 min; MS (ESIpos): m/z=352.2 [M+H]⁺.

### Intermediate 374

### tert-butyl (7RS)-2-(4-fluorophenyl)-7-[2-(methylamino)-2-oxoethyl]-3-(pyridin-4-yl)-6,7-dihydropyrazolo[1,5-a]pyrazine-5(4H)-carboxylate

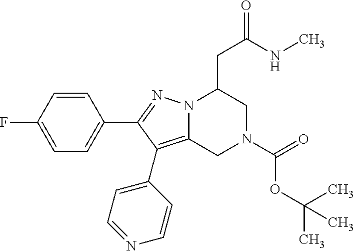 [SMILES: CNC(=O)CC1CN(C(=O)OC(C)(C)C)Cc2c(-c3ccncc3)c(-c3ccc(F)cc3)nn21]

To a solution of [(7RS)-5-(tert-butoxycarbonyl)-2-(4-fluorophenyl)-3-(pyridin-4-yl)-4,5,6,7-tetrahydropyrazolo[1,5-a]pyrazin-7-yl]acetic acid (150 mg, 331 μmol, Intermediate 371) and methylamine hydrochloride (33.6 mg, 497 μmol) in N,N-dimethylformamide (2 ml) were added O-(7-azabenzotriazol-1-yl)-N,N,N′,N′-tetramethyluronium hexafluorophosphate (315 mg, 829 μmol) and N,N-diisopropylethylamine (107 mg, 829 μmol) at 25° C. The mixture was stirred at 25° C. for 16 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by preparative TLC (petroleum ether:ethyl acetate=0:1) to give tert-butyl (7RS)-2-(4-fluorophenyl)-7-[2-(methylamino)-2-oxoethyl]-3-(pyridin-4-yl)-6,7-dihydropyrazolo[1,5-a]pyrazine-5(4H)-carboxylate (53 mg, 84% purity, 35% yield) as a colorless oil.

LC-MS (Method 15): Rₜ=0.757 min; MS (ESIpos): m/z=466.2 [M+H]⁺

### Intermediate 375

### 2-[(7RS)-2-(4-fluorophenyl)-3-(pyridin-4-yl)-4,5,6,7-tetrahydropyrazolo[1,5-a]pyrazin-7-yl]-N-methylacetamide hydrogen chloride (1/1)

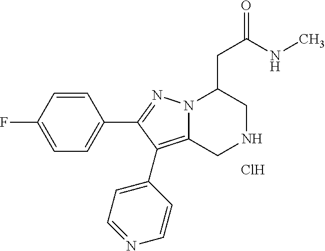 [SMILES: CNC(=O)CC1CNCc2c(-c3ccncc3)c(-c3ccc(F)cc3)nn21 ; Cl]

The solution of tert-butyl (7RS)-2-(4-fluorophenyl)-7-[2-(methylamino)-2-oxoethyl]-3-(pyridin-4-yl)-6,7-dihydropyrazolo[1,5-a]pyrazine-5(4H)-carboxylate (64.0 mg, 94% purity, 129 μmol, Intermediate 374) in hydrochloric acid (2 ml, 4M in 1,4-dioxane) was stirred at 25° C. for 3 hours. The reaction mixture was concentrated under reduced pressure to give 2-[(7RS)-2-(4-fluorophenyl)-3-(pyridin-4-yl)-4,5,6,7-tetrahydropyrazolo[1,5-a]pyrazin-7-yl]-N-methylacetamide hydrogen chloride (1/1) (53 mg, 86% purity, 98% yield) as a colorless oil.

LC-MS (Method 15): Rₜ=0.728 min; MS (ESIpos): m/z=366.2 [M+H]⁺.

### Intermediate 376

### tert-butyl (7RS)-7-[2-(dimethylamino)-2-oxoethyl]-2-(4-fluorophenyl)-3-(pyridin-4-yl)-6,7-dihydropyrazolo[1,5-a]pyrazine-5(4H)-carboxylate

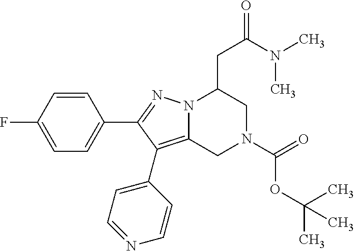 [SMILES: CN(C)C(=O)CC1CN(C(=O)OC(C)(C)C)Cc2c(-c3ccncc3)c(-c3ccc(F)cc3)nn21]

To a solution of [(7RS)-5-(tert-butoxycarbonyl)-2-(4-fluorophenyl)-3-(pyridin-4-yl)-4,5,6,7-tetrahydropyrazolo[1,5-a]pyrazin-7-yl]acetic acid (150 mg, 331 μmol Intermediate 371) and dimethylammonium chloride (40.5 mg, 497 μmol) in N,N-dimethylformamide (3 ml) were added O-(7-azabenzotriazol-1-yl)-N,N,N′,N′-tetramethyluronium hexafluorophosphate (189 mg, 497 μmol) and N,N-diisopropylethylamine (120 μl, 660 μmol) at 25° C. The mixture was stirred at 25° C. for 16 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by preparative TLC (petroleum ether:ethyl acetate=0:1) to give tert-butyl (7RS)-7-[2-(dimethylamino)-2-oxoethyl]-2-(4-fluorophenyl)-3-(pyridin-4-yl)-6,7-dihydropyrazolo[1,5-a]pyrazine-5(4H)-carboxylate (65 mg, 93% purity, 49% yield) as a colorless oil.

LC-MS (Method 14): Rₜ=0.711 min; MS (ESIpos): m/z=480.3 [M+H]⁺.

### Intermediate 377

### 2-[(7RS)-2-(4-fluorophenyl)-3-(pyridin-4-yl)-4,5,6,7-tetrahydropyrazolo[1,5-a]pyrazin-7-yl]-N,N-dimethylacetamide hydrogen chloride (1/1)

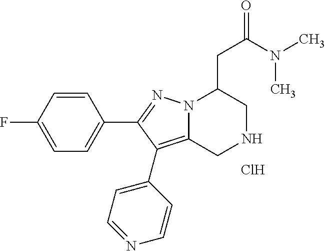 [SMILES: CN(C)C(=O)CC1CNCc2c(-c3ccncc3)c(-c3ccc(F)cc3)nn21 ; Cl]

The solution of tert-butyl (7RS)-7-[2-(dimethylamino)-2-oxoethyl]-2-(4-fluorophenyl)-3-(pyridin-4-yl)-6,7-dihydropyrazolo[1,5-a]pyrazine-5(4H)-carboxylate (65.0 mg, 136 μmol, Intermediate 376) in hydrochloric acid (2.0 ml, 4M in 1,4-dioxane) was stirred at 25° C. for 3 hours. The reaction mixture was concentrated under reduced pressure to give 2-[(7RS)-2-(4-fluorophenyl)-3-(pyridin-4-yl)-4,5,6,7-tetrahydropyrazolo[1,5-a]pyrazin-7-yl]-N,N-dimethylacetamide hydrogen chloride (1/1) (61 mg, 79% purity, 68% yield) as a colorless oil.

LC-MS (Method 14): Rₜ=0.963 min; MS (ESIpos): m/z=372.1 [M+H]⁺

### Intermediate 378

### Ethyl 3-bromo-1-{(2RS)-2-[(tert-butoxycarbonyl)amino]propyl}-1H-pyrazole-5-carboxylate

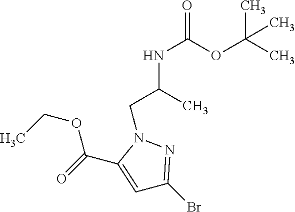 [SMILES: CCOC(=O)c1cc(Br)nn1CC(C)NC(=O)OC(C)(C)C]

To a solution of ethyl 3-bromo-1H-pyrazole-5-carboxylate (10.0 g, 45.7 mmol) in tetrahydrofuran (100 ml) were added rac-tert-butyl [(2RS)-1-hydroxypropan-2-yl]carbamate (8.00 g, 45.7 mmol), triphenylphosphine (23.9 g, 91.3 mmol) and diisopropyl azodicarboxylate (18.5 g, 91.3 mmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=5:1) to give ethyl 3-bromo-1-{(2RS)-2-[(tert-butoxycarbonyl)amino]propyl}-1H-pyrazole-5-carboxylate (17.0 g, 93% purity, 45.7 mmol, 99% yield) as a white solid.

LC-MS (Method 14): Rₜ=0.650 min; MS (ESIpos): m/z=278.0 [M+H]⁺.

¹H NMR (400 MHz, DMSO-d₆), δ [ppm]=6.82 (s, 1H), 5.11-5.01 (m, 1H), 4.67-4.54 (m, 1H), 4.51-4.41 (m, 1H), 4.39-4.30 (m, 2H), 1.36 (s, 9H), 1.31 (d, J=6.4 Hz, 1H), 1.29 (d, J=6.4 Hz, 3H), 1.15 (d, J=6.8 Hz, 3H).

### Intermediate 379

### tert-butyl (RS)-(1-(3-bromo-5-(hydroxymethyl)-1H-pyrazol-1-yl) propan-2-yl) carbamate

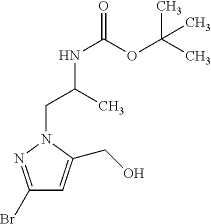 [SMILES: CC(Cn1nc(Br)cc1CO)NC(=O)OC(C)(C)C]

To a solution of rac-ethyl 3-bromo-1-{(2RS)-2-[(tert-butoxycarbonyl)amino]propyl}-1H-pyrazole-5-carboxylate (10.0 g, 93% purity, 24.7 mmol Intermediate 378) in ethanol (100 ml) were added sodium tetrahydroborate (2.81 g, 74.2 mmol) at 0° C. under nitrogen atmosphere. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give tert-butyl (RS)-(1-(3-bromo-5-(hydroxymethyl)-1H-pyrazol-1-yl) propan-2-yl) carbamate (8.75 g, 88% purity, 93% yield) as a colourless oil.

LC-MS (Method 15): Rₜ=0.782 min; MS (ESIpos): m/z=234.0 [M+H]⁺.

¹H NMR (400 MHz, DMSO-d₆), δ [ppm]=6.49 (s, 1H), 6.23 (s, 1H), 5.02-4.93 (m, 1H), 4.63 (d, J=4.8 Hz, 2H), 4.11-4.05 (m, 1H), 1.37 (s, 9H), 1.26 (d, J=6.0 Hz, 3H).

### Intermediate 380

### N-[(3-bromo-1-{(2RS)-2-[(tert-butoxycarbonyl)amino]propyl}-1H-pyrazol-5-yl)methyl]-N,N-diethylethanaminium methanesulfonate

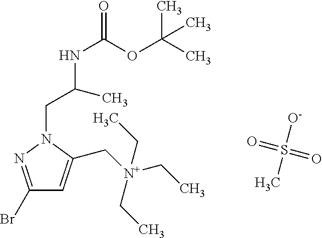

To a solution of tert-butyl (RS)-(1-(3-bromo-5-(hydroxymethyl)-1H-pyrazol-1-yl) propan-2-yl) carbamate (8.30 g, 93% purity, 23.1 mmol, Intermediate 379) in dichloromethane (83 ml) was added triethylamine (19 ml, 140 mmol) and methanesulfonic anhydride (8.05 g, 46.2 mmol) at 0° C. under nitrogen atmosphere. The mixture was stirred at 25° C. for 16 hours. The reaction mixture was concentrated in vacuo to give N-[(3-bromo-1-{(2RS)-2-[(tert-butoxycarbonyl)amino]propyl}-1H-pyrazol-5-yl)methyl]-N,N-diethylethanaminium methanesulfonate (11.0 g, 90% purity, 83% yield) as a brown oil.

LC-MS (Method 15): Rₜ=0.735 min; MS (ESIpos): m/z=419.2 [M+H]⁺.

### Intermediate 381

### tert-butyl 2-bromo-6-methyl-6,7-dihydropyrazolo[1,5-a]pyrazine-5(4H)-carboxylate

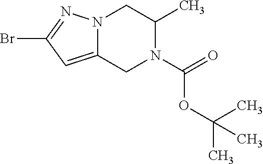 [SMILES: CC1Cn2nc(Br)cc2CN1C(=O)OC(C)(C)C]

To a solution of N-[(3-bromo-1-{(2RS)-2-[(tert-butoxycarbonyl)amino]propyl}-1H-pyrazol-5-yl)methyl]-N,N-diethylethanaminium (11.0 g, 90% purity, 23.7 mmol, Intermediate 380) in N,N-dimethylformamide (90 ml) and tetrahydrofuran (30 ml) was added sodium hydride (5.68 g, 60% purity, 142 mmol) at 0° C. under nitrogen atmosphere. The mixture was stirred at 25° C. for 16 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=5:1) to give tert-butyl (RS)-2-bromo-6-methyl-6,7-dihydropyrazolo[1,5-a]pyrazine-5(4H)-carboxylate (6.38 g, 96% purity, 82% yield) as a yellow oil.

LC-MS (Method 14): Rₜ=0.620 min; MS (ESIpos): m/z=316.1 [M+H]⁺.

¹H NMR (400 MHz, DMSO-d6), δ [ppm]=6.10 (s, 1H), 4.94 (d, J=17.2 Hz, 1H), 4.81 (s, 1H), 4.30-4.15 (m, 2H), 4.07-4.02 (m, 1H), 1.50 (s, 9H), 1.20 (d, J=7.2 Hz, 3H).

### Intermediate 382

### tert-butyl 2-bromo-3-iodo-6-methyl-6,7-dihydropyrazolo[1,5-a]pyrazine-5(4H)-carboxylate

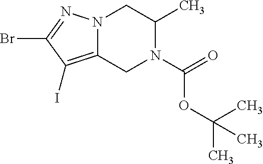 [SMILES: CC1Cn2nc(Br)c(I)c2CN1C(=O)OC(C)(C)C]

To a solution of tert-butyl 2-bromo-6-methyl-6,7-dihydropyrazolo[1,5-a]pyrazine-5(4H)-carboxylate (4.38 g, 96% purity, 13.3 mmol, Intermediate 381) in dichloromethane (15 ml) and methanol (30 ml) was added N-iodosuccinimide (5.98 g, 26.6 mmol) at 25° C. The mixture was stirred at 45° C. for 16 hours. The reaction mixture was poured into saturated sodium thiosulfate aqueous solution and extracted with ethyl acetate. The combined organic phase is washed with brine, dried on anhydrous sodium sulfate, filtered and concentrated in vacuo to give a residue. The residue was purified by column chromatography on silica gel (100~200 mesh, petroleum ether:ethyl acetate=20:1 to 10:1) to give tert-butyl 2-bromo-3-iodo-6-methyl-6,7-dihydropyrazolo[1,5-a]pyrazine-5(4H)-carboxylate (5.21 g, 94% purity, 83% yield) as a yellow oil.

LC-MS (Method 16): Rₜ=0.983 min; MS (ESIpos): m/z=444.0 [M+H]⁺.

¹H NMR (400 MHz, DMSO-d6), δ [ppm]=4.83 (d, J=16.0 Hz, 2H), 4.21 (dd, J=12.4, 4.8 Hz, 1H), 4.08-4.01 (m, 2H), 1.51 (s, 9H), 1.19 (d, J=7.2 Hz, 3H).

### Intermediate 383

### tert-butyl 2-bromo-6-methyl-3-(pyridin-4-yl)-6,7-dihydropyrazolo[1,5-a]pyrazine-5(4H)-carboxylate

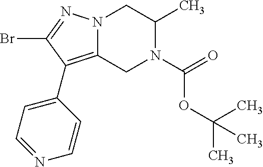 [SMILES: CC1Cn2nc(Br)c(-c3ccncc3)c2CN1C(=O)OC(C)(C)C]

To a solution of tert-butyl 2-bromo-3-iodo-6-methyl-6,7-dihydropyrazolo[1,5-a]pyrazine-5(4H)-carboxylate (4.81 g, 94% purity, 10.2 mmol, Intermediate 382) and pyridin-4-ylboronic acid (1.26 g, 10.2 mmol) in 1,4-dioxane (40 ml) and water (10 ml) were added sodium carbonate (2.71 g, 25.6 mmol) and (1,1′-bis(diphenylphosphino) ferrocene) palladium (II) dichloride (748 mg, 1.02 mmol) at 25° C. The mixture was stirred at 80° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give tert-butyl 2-bromo-6-methyl-3-(pyridin-4-yl)-6,7-dihydropyrazolo[1,5-a]pyrazine-5(4H)-carboxylate (3.50 g, 95% purity, 83% yield) as a colourless oil.

LC-MS (Method 14): Rₜ=0.473 min; MS (ESIpos): m/z=393.0 [M+H]⁺

### Intermediate 384

### tert-butyl 2-(4-chloro-2-fluorophenyl)-6-methyl-3-(pyridin-4-yl)-6,7-dihydropyrazolo[1,5-a]pyrazine-5(4H)-carboxylate

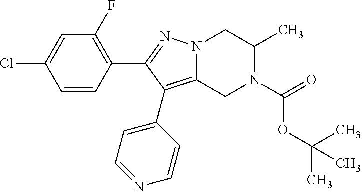 [SMILES: CC1Cn2nc(-c3ccc(Cl)cc3F)c(-c3ccncc3)c2CN1C(=O)OC(C)(C)C]

To a solution of racemic tert-butyl 2-bromo-6-methyl-3-(pyridin-4-yl)-6,7-dihydropyrazolo[1,5-a]pyrazine-5(4H)-carboxylate (210 mg, 94% purity, 502 μmol, Intermediate 383) and (4-chloro-2-fluorophenyl) boronic acid (123 mg, 703 μmol) in 1,4-dioxane (3.0 ml) and water (1.0 ml) were added sodium carbonate (106 mg, 1.00 mmol) and (1,1′-bis(diphenylphosphino) ferrocene) palladium (II) dichloride (36.7 mg, 50.2 μmol) at 25° C. The mixture was stirred at 90° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give tert-butyl 2-(4-chloro-2-fluorophenyl)-6-methyl-3-(pyridin-4-yl)-6,7-dihydropyrazolo[1,5-a]pyrazine-5(4H)-carboxylate (178 mg, 80% purity, 64% yield) as a colourless oil.

LC-MS (Method 15): Rₜ=0.813 min; MS (ESIpos): m/z=443.1 [M+H]⁺.

### Intermediate 385

### 2-(4-chloro-2-fluorophenyl)-6-methyl-3-(pyridin-4-yl)-4,5,6,7-tetrahydropyrazolo[1,5-a]pyrazine hydrogen chloride (1/1)

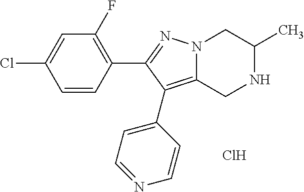 [SMILES: CC1Cn2nc(-c3ccc(Cl)cc3F)c(-c3ccncc3)c2CN1 ; Cl]

The solution of rac-tert-butyl (RS)-2-(4-chloro-2-fluorophenyl)-6-methyl-3-(pyridin-4-yl)-6,7-dihydropyrazolo[1,5-a]pyrazine-5(4H)-carboxylate (100 mg, 94% purity, 212 μmol, Intermediate 384) in hydrochloric acid (4M in dioxane, 1 ml) was stirred at 25° C. for 3 hours. The reaction mixture was concentrated under reduced pressure to give racemic 2-(4-chloro-2-fluorophenyl)-6-methyl-3-(pyridin-4-yl)-4,5,6,7-tetrahydropyrazolo[1,5-a]pyrazine hydrogen chloride (1/1) (80.0 mg, 89% purity, 88% yield) as a colorless oil.

LC-MS (Method 14): Rₜ=0.378 min; MS (ESIpos): m/z=343.1 [M+H]⁺.

### Intermediate 386

### tert-butyl 2-(4-chloro-2-fluorophenyl)-6-methyl-3-(pyridin-4-yl)-6,7-dihydropyrazolo[1,5-a]pyrazine-5-carboxylate (isomer 2)

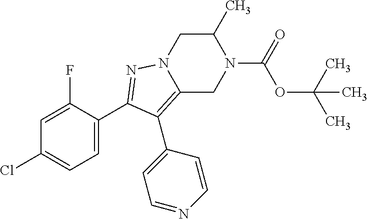 [SMILES: CC1Cn2nc(-c3ccc(Cl)cc3F)c(-c3ccncc3)c2CN1C(=O)OC(C)(C)C]

Rac-tert-butyl (RS)-2-(4-chloro-2-fluorophenyl)-6-methyl-3-(pyridin-4-yl)-6,7-dihydropyrazolo[1,5-a]pyrazine-5(4H)-carboxylate (359 mg, 94% purity, 0.761 mmol, Intermediate 384) was separated by preparative-SFC (Instrument: Waters 80Q SFC; Column: DAICEL CHIRALPAK AD-H (250 mm*30 mm, 5 μm); “Sample preparation: Add CH₃OH 20 ml into sample Instrument: Thar 80 SFC Mobile Phase: 15% MEOH (0.1% NH₃·H₂O) in Supercritical CO2 Flow Rate: 50 g/min Cycle Time: 5.5 min, total time: 60 min Single injection volume: 2 ml Back Pressure: 100 bar to keep the CO2 in Supercritical flow”; temperature: room temperature; Detector: UV 220 nm) to give (tert-butyl(2-(4-chloro-2-fluorophenyl)-6-methyl-3-(pyridin-4-yl)-6,7-dihydropyrazolo[1,5-a]pyrazine-5(4H)-carboxylate (isomer 1, first eluting, SFC retention time: 0.980 min) as a white solid (100 mg, 95% purity, 28% yield) and (tert-butyl-2-(4-chloro-2-fluorophenyl)-6-methyl-3-(pyridin-4-yl)-6,7-dihydropyrazolo[1,5-a]pyrazine-5(4H)-carboxylate (isomer 2, second eluting, SFC retention time: 1.122 min) (117 mg, 95% purity, 32% yield) as a white solid.

LC-MS (Method 14): Rₜ=0.814 minute; MS (ESIpos): m/z=443.1 [M+H]⁺.

### Intermediate 387

### 2-(4-chloro-2-fluorophenyl)-6-methyl-3-(pyridin-4-yl)-4,5,6,7-tetrahydropyrazolo[1,5-a]pyrazine hydrogen chloride (1/1) (isomer 1)

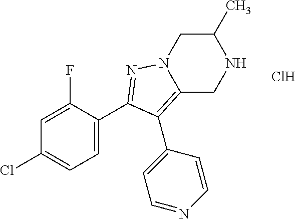 [SMILES: CC1Cn2nc(-c3ccc(Cl)cc3F)c(-c3ccncc3)c2CN1 ; Cl]

The solution of racemic tert-butyl 2-(4-chloro-2-fluorophenyl)-6-methyl-3-(pyridin-4-yl)-6,7-dihydropyrazolo[1,5-a]pyrazine-5-carboxylate (isomer 1) (100 mg, 95% purity, 214 μmol, Intermediate 386 in hydrochloric acid (4M in dioxane, 1.0 ml) was stirred at 25° C. for 3 hours. The mixture was concentrated under reduced pressure to give 2-(4-chloro-2-fluorophenyl)-6-methyl-3-(pyridin-4-yl)-4,5,6,7-tetrahydropyrazolo[1,5-a]pyrazine hydrogen chloride (1/1) (isomer 2) (80.0 mg, 86% purity, 85% yield) as a colorless oil.

LC-MS (Method 16): Rₜ=0.756 min; MS (ESIpos): m/z=343.2 [M+H]⁺.

### Intermediate 388

### 2-(4-chloro-2-fluorophenyl)-6-methyl-3-(pyridin-4-yl)-4,5,6,7-tetrahydropyrazolo[1,5-a]pyrazine hydrogen chloride (1/1) (isomer 2)

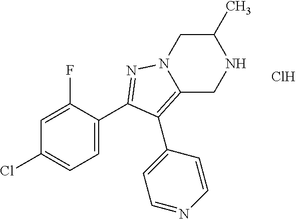 [SMILES: CC1Cn2nc(-c3ccc(Cl)cc3F)c(-c3ccncc3)c2CN1 ; Cl]

The solution of tert-butyl-2-(4-chloro-2-fluorophenyl)-6-methyl-3-(pyridin-4-yl)-6,7-dihydropyrazolo[1,5-a]pyrazine-5(4H)-carboxylate (isomer 2) (117 mg, 95% purity, 251 μmol, Intermediate 386) in hydrochloric acid (4M in dioxane, 1.0 ml) was stirred at 25° C. for 3 hours. The mixture was concentrated under reduced pressure to give 2-(4-chloro-2-fluorophenyl)-6-methyl-3-(pyridin-4-yl)-4,5,6,7-tetrahydropyrazolo[1,5-a]pyrazine hydrogen chloride (1/1) (isomer 2) (90.0 mg, 80% purity, 76% yield) as a colorless oil.

LC-MS (Method 16): Rₜ=0.757 min; MS (ESIpos): m/z=343.2 [M+H]⁺

### Intermediate 389

### rac-tert-butyl (RS)-2-(4-methoxyphenyl)-6-methyl-3-(pyridin-4-yl)-6,7-dihydropyrazolo[1,5-a]pyrazine-5(4H)-carboxylate

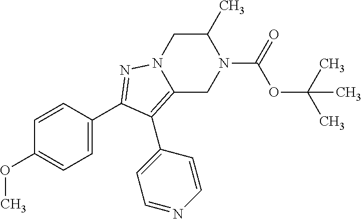 [SMILES: COc1ccc(-c2nn3c(c2-c2ccncc2)CN(C(=O)OC(C)(C)C)C(C)C3)cc1]

To a solution of rac-tert-butyl (RS)-2-bromo-6-methyl-3-(pyridin-4-yl)-6,7-dihydropyrazolo[1,5-a]pyrazine-5(4H)-carboxylate (250 mg, 95% purity, 604 μmol, Intermediate 383) and (4-methoxyphenyl) boronic acid (128 mg, 845 μmol) in 1,4-dioxane (4.0 ml) and water (1.0 ml) were added sodium carbonate (128 mg, 1.21 mmol) and (1,1′-bis(diphenylphosphino) ferrocene) palladium (II) dichloride (44.2 mg, 60.4 μmol) at 25° C. The mixture was stirred at 90° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give rac-tert-butyl (RS)-2-(4-methoxyphenyl)-6-methyl-3-(pyridin-4-yl)-6,7-dihydropyrazolo[1,5-a]pyrazine-5(4H)-carboxylate (200 mg, 65% purity, 51% yield) as a colourless oil.

LC-MS (Method 14): Rₜ=0.488 min; MS (ESIpos): m/z=421.4 [M+H]⁺.

### Intermediate 390

### 2-(4-methoxyphenyl)-6-methyl-3-(pyridin-4-yl)-4,5,6,7-tetrahydropyrazolo[1,5-a]pyrazine hydrogen chloride (1/1)

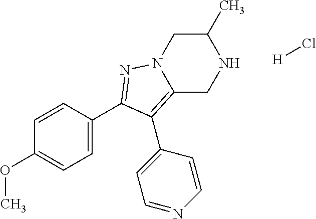 [SMILES: COc1ccc(-c2nn3c(c2-c2ccncc2)CNC(C)C3)cc1 ; Cl]

The solution of tert-butyl (RS)-2-(4-methoxyphenyl)-6-methyl-3-(pyridin-4-yl)-6,7-dihydropyrazolo[1,5-a]pyrazine-5(4H)-carboxylate (200 mg, 65% purity, 309 μmol, Intermediate 389) in hydrochloric acid (4M in dioxane, 1.0 ml) was stirred at 25° C. for 3 hours. The mixture was concentrated under reduced pressure to give racemic 2-(4-methoxyphenyl)-6-methyl-3-(pyridin-4-yl)-4,5,6,7-tetrahydropyrazolo[1,5-a]pyrazine hydrogen chloride (110 mg, 97% purity, 97% yield) as a colorless oil.

LC-MS (Method 16): Rₜ=0.702 min; MS (ESIpos): m/z=321.2 [M+H]⁺.

### Intermediate 391

### 6-bromo-2-fluoro-1-benzothiophene

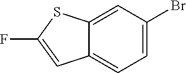 [SMILES: Fc1cc2ccc(Br)cc2s1]

To a solution of 6-bromo-1-benzothiophene (1.00 g, 4.69 mmol) in tetrahydrofuran (10 ml) was added dropwise lithium diisopropylamide (2 M in hexane and tetrahydrofuran, 4.7 ml, 9.40 mmol) at −70° C. under nitrogen atmosphere. After stirring at −70° C. for 0.5 hour, to the mixture above was added dropwise N-(benzenesulfonyl)-N-fluorobenzenesulfonamide (2.96 g, 9.39 mmol) at −70° C. and the resulting mixture was stirred at 20° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=1:0) to give 6-bromo-2-fluorobenzo[b]thiophene (300 mg, 80% purity, 22% yield) as a colorless oil.

¹H NMR (400 MHz, DMSO-d6), δ [ppm]=7.81 (d, J=0.8 Hz, 1H), 7.51-7.48 (m, 1H), 7.48-7.44 (m, 1H), 6.69 (d, J=2.4 Hz, 1H).

### Intermediate 392

### 2-(2-fluoro-1-benzothiophen-6-yl)-4,4,5,5-tetramethyl-1,3,2-dioxaborolane

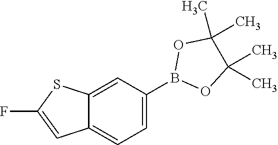 [SMILES: CC1(C)OB(c2ccc3cc(F)sc3c2)OC1(C)C]

To a solution of 6-bromo-2-fluoro-1-benzothiophene (300 mg, 80% purity, 1.04 mmol, Intermediate 391) in 1,4-dioxane (5.0 ml) were added 4,4,4′,4′,5,5,5′,5′-octamethyl-2,2′-bi-1,3,2-dioxaborolane (316 mg, 1.25 mmol), potassium acetate (204 mg, 2.08 mmol) and [1,1′-bis(diphenylphosphino) ferrocene]palladium (II) dichloride (76.0 mg, 104 μmol) at 25° C. The mixture was stirred at 90° C. for 16 hours under nitrogen atmosphere. The reaction mixture was filtered through a pad of celite and the filtrate was concentrated in vacuo to give a residue. The residue was purified by column chromatography on silica gel (100~200 mesh, petroleum ether:ethyl acetate=1:0) to give 2-(2-fluoro-1-benzothiophen-6-yl)-4,4,5,5-tetramethyl-1,3,2-dioxaborolane (170 mg, 90% purity, 53% yield) as a colorless oil.

¹H NMR (400 MHz, DMSO-d₆), δ [ppm]=8.15 (s, 1H), 7.77 (d, J=8.0 Hz, 1H), 7.62 (d, J=8.0 Hz, 1H), 6.72 (d, J=2.4 Hz, 1H), 1.37 (s, 12H).

### Intermediate 393

### rac-tert-butyl (RS)-2-(2-fluoro-1-benzothiophen-6-yl)-6-methyl-3-(pyridin-4-yl)-6,7-dihydropyrazolo[1,5-a]pyrazine-5(4H)-carboxylate

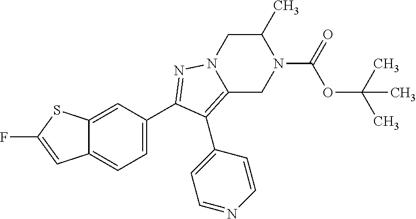 [SMILES: CC1Cn2nc(-c3ccc4cc(F)sc4c3)c(-c3ccncc3)c2CN1C(=O)OC(C)(C)C]

To a solution of 2-(2-fluoro-1-benzothiophen-6-yl)-4,4,5,5-tetramethyl-1,3,2-dioxaborolane (150 mg, 90% purity, 485 μmol, Intermediate 392) in 1,4-dioxane (3.0 ml) and water (1.0 ml) were added rac-tert-butyl (RS)-2-bromo-6-methyl-3-(pyridin-4-yl)-6,7-dihydropyrazolo[1,5-a]pyrazine-5(4H)-carboxylate (201 mg, 95% purity, 485 μmol, Intermediate 383), sodium carbonate (103 mg, 971 μmol) and [1,1′-bis(diphenylphosphino) ferrocene]palladium (II) dichloride (35.5 mg, 48.5 μmol) at 25° C. The mixture was stirred at 100° C. for 16 hours under nitrogen atmosphere. The reaction mixture was filtered through a pad of celite and the filtrate was concentrated in vacuo to give a residue. The residue was purified by column chromatography on silica gel (100~200 mesh, petroleum ether:ethyl acetate=1:1) to give rac-tert-butyl (RS)-2-(2-fluoro-1-benzothiophen-6-yl)-6-methyl-3-(pyridin-4-yl)-6,7-dihydropyrazolo[1,5-a]pyrazine-5(4H)-carboxylate (150 mg, 70% purity, 47% yield) as a colorless oil.

LC-MS (Method 16): Rₜ=0.566 min; MS (ESIpos): m/z=465.3 [M+H]⁺

### Intermediate 394

### 2-(2-fluoro-1-benzothiophen-6-yl)-6-methyl-3-(pyridin-4-yl)-4,5,6,7-tetrahydropyrazolo[1,5-a]pyrazine hydrogen chloride (1/1)

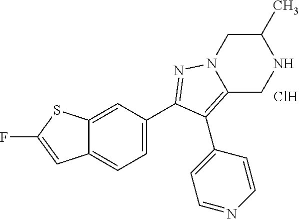 [SMILES: CC1Cn2nc(-c3ccc4cc(F)sc4c3)c(-c3ccncc3)c2CN1 ; Cl]

The solution of racemic tert-butyl (RS)-2-(2-fluoro-1-benzothiophen-6-yl)-6-methyl-3-(pyridin-4-yl)-6,7-dihydropyrazolo[1,5-a]pyrazine-5(4H)-carboxylate (150 mg, 70% purity, 226 μmol, Intermediate 393) in hydrochloric acid (4M in ethyl acetate, 3.0 ml, 12.0 mmol) was stirred at 25° C. for 3 hours. The reaction mixture was concentrated in vacuo to give 2-(2-fluoro-1-benzothiophen-6-yl)-6-methyl-3-(pyridin-4-yl)-4,5,6,7-racemic tetrahydropyrazolo[1,5-a]pyrazine hydrogen chloride (1/1) (100 mg, 65% purity, 72% yield) as a yellow solid.

LC-MS (Method 16): Rₜ=0.469 min; MS (ESIpos): m/z=365.2 [M+H]⁺.

### Intermediate 395

### tert-butyl (6RS)-2-[2-fluoro-4-(trifluoromethyl)phenyl]-6-methyl-3-(pyridin-4-yl)-6,7-dihydropyrazolo[1,5-a]pyrazine-5(4H)-carboxylate

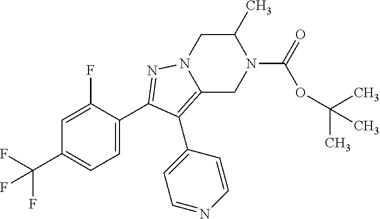 [SMILES: CC1Cn2nc(-c3ccc(C(F)(F)F)cc3F)c(-c3ccncc3)c2CN1C(=O)OC(C)(C)C]

To a solution of racemic tert-butyl (6RS)-2-bromo-6-methyl-3-(pyridin-4-yl)-6,7-dihydropyrazolo[1,5-a]pyrazine-5(4H)-carboxylate (150 mg, 95% purity, 362 μmol, Intermediate 383) and [2-fluoro-4-(trifluoromethyl)phenyl]boronic acid (151 mg, 725 μmol) in 1,4-dioxane (3.0 ml) and water (1.5 ml) were added sodium carbonate (96.0 mg, 906 μmol) and [1,1′-bis(diphenylphosphino) ferrocene]palladium (II) dichloride (53.0 mg, 72.5 μmol) at 25° C. The mixture was stirred at 90° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give racemic tert-butyl (RS)-2-(2-fluoro-4-(trifluoromethyl)phenyl)-6-methyl-3-(pyridin-4-yl)-6,7-dihydropyrazolo[1,5-a]pyrazine-5(4H)-carboxylate (209 mg, 99% purity, 120% yield) as a colourless oil.

LC-MS (Method 15): Rₜ=0.827 min; MS (ESIpos): m/z=477.1 [M+H]⁺

### Intermediate 396

### rac-(RS)-2-[2-fluoro-4-(trifluoromethyl)phenyl]-6-methyl-3-(pyridin-4-yl)-4,5,6,7-tetrahydropyrazolo[1,5-a]pyrazine hydrogen chloride (1/1)

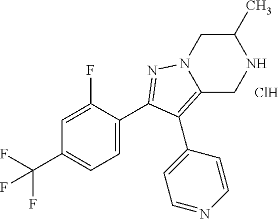 [SMILES: CC1Cn2nc(-c3ccc(C(F)(F)F)cc3F)c(-c3ccncc3)c2CN1 ; Cl]

The solution of rac-tert-butyl (6RS)-2-[2-fluoro-4-(trifluoromethyl)phenyl]-6-methyl-3-(pyridin-4-yl)-6,7-dihydropyrazolo[1,5-a]pyrazine-5(4H)-carboxylate (205 mg, 99% purity, 426 μmol, Intermediate 395) in hydrochloric acid (4M in dioxane, 2.0 ml) was stirred at 25° C. for 3 hours. The mixture was concentrated under reduced pressure to give rac-(RS)-2-(2-fluoro-4-(trifluoromethyl)phenyl)-6-methyl-3-(pyridin-4-yl)-4,5,6,7-tetrahydropyrazolo[1,5-a]pyrazine hydrogen chloride (1/1) (150 mg, 98% purity, 84% yield) as a colorless oil.

LC-MS (Method 17): Rₜ=0.619 min; MS (ESIpos): m/z=377.1 [M+H]⁺.

### Intermediate 397

### rac-tert-butyl (RS)-2-(2-fluoro-4-methoxyphenyl)-6-methyl-3-(pyridin-4-yl)-6,7-dihydropyrazolo[1,5-a]pyrazine-5(4H)-carboxylate

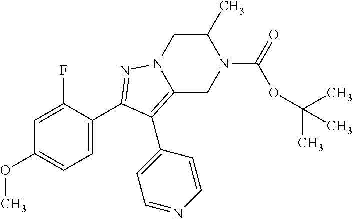 [SMILES: COc1ccc(-c2nn3c(c2-c2ccncc2)CN(C(=O)OC(C)(C)C)C(C)C3)c(F)c1]

To a solution of rac-tert-butyl (RS)-2-bromo-6-methyl-3-(pyridin-4-yl)-6,7-dihydropyrazolo[1,5-a]pyrazine-5(4H)-carboxylate (150 mg, 95% purity, 362 μmol, Intermediate 383) and (2-fluoro-4-methoxyphenyl) boronic acid (92.4 mg, 543 μmol) in 1,4-dioxane (3.0 ml) and water (1.0 ml) were added sodium carbonate (76.8 mg, 725 μmol) and [1,1′-bis(diphenylphosphino) ferrocene]palladium (II) dichloride (26.5 mg, 36.2 μmol) at 25° C. The mixture was stirred at 90° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give rac-tert-butyl (RS)-2-(2-fluoro-4-methoxyphenyl)-6-methyl-3-(pyridin-4-yl)-6,7-dihydropyrazolo[1,5-a]pyrazine-5(4H)-carboxylate (173 mg, 96% purity, 105% yield) as a colourless oil.

LC-MS (Method 16): Rₜ=0.834 min; MS (ESIpos): m/z=439.2 [M+H]⁺.

### Intermediate 398

### rac-(RS)-2-(2-fluoro-4-methoxyphenyl)-6-methyl-3-(pyridin-4-yl)-4,5,6,7-tetrahydropyrazolo[1,5-a]pyrazine hydrogen chloride (1/1)

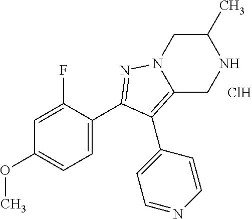 [SMILES: COc1ccc(-c2nn3c(c2-c2ccncc2)CNC(C)C3)c(F)c1 ; Cl]

The solution of rac-tert-butyl (RS)-2-(2-fluoro-4-methoxyphenyl)-6-methyl-3-(pyridin-4-yl)-6,7-dihydropyrazolo[1,5-a]pyrazine-5(4H)-carboxylate (173 mg, 96% purity, 379 μmol, Intermediate 397) in hydrochloric acid (4M in dioxane, 1.9 ml) was stirred at 25° C. for 3 hours. The mixture was concentrated under reduced pressure to give rac-(RS)-2-(2-fluoro-4-methoxyphenyl)-6-methyl-3-(pyridin-4-yl)-4,5,6,7-tetrahydropyrazolo[1,5-a]pyrazine (130 mg, 96% purity, 88% yield) as a colorless oil.

LC-MS (Method 20): Rₜ=0.827 min; MS (ESIpos): m/z=339.1 [M+H]⁺.

### Intermediate 399

### rac-tert-butyl (RS)-6-methyl-2-(naphthalen-2-yl)-3-(pyridin-4-yl)-6,7-dihydropyrazolo[1,5-a]pyrazine-5(4H)-carboxylate

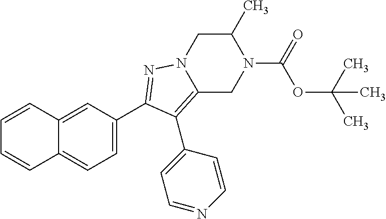 [SMILES: CC1Cn2nc(-c3ccc4ccccc4c3)c(-c3ccncc3)c2CN1C(=O)OC(C)(C)C]

To a solution of rac-tert-butyl (6RS)-2-bromo-6-methyl-3-(pyridin-4-yl)-6,7-dihydropyrazolo[1,5-a]pyrazine-5(4H)-carboxylate (150 mg, 95% purity, 362 μmol, Intermediate 383) and naphthalen-2-ylboronic acid (125 mg, 725 μmol) in 1,4-dioxane (3.0 ml) and water (1.0 ml) were added sodium carbonate (76.8 mg, 725 μmol) and [1,1-bis(diphenylphosphino) ferrocene]palladium (II) dichloride (53.0 mg, 72.5 μmol) at 25° C. The mixture was stirred at 90° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give rac-tert-butyl (RS)-6-methyl-2-(naphthalen-2-yl)-3-(pyridin-4-yl)-6,7-dihydropyrazolo[1,5-a]pyrazine-5(4H)-carboxylate (170 mg, 53% purity, 56% yield) as a colourless oil.

LC-MS (Method 16): Rₜ=0.882 min; MS (ESIpos): m/z=441.3 [M+H]⁺.

### Intermediate 400

### rac-(RS)-6-methyl-2-(naphthalen-2-yl)-3-(pyridin-4-yl)-4,5,6,7-tetrahydropyrazolo[1,5-a]pyrazine hydrogen chloride (1/1)

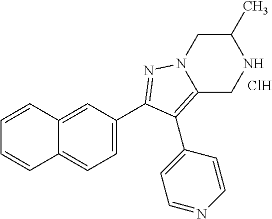 [SMILES: CC1Cn2nc(-c3ccc4ccccc4c3)c(-c3ccncc3)c2CN1 ; Cl]

The solution of rac-tert-butyl (RS)-6-methyl-2-(naphthalen-2-yl)-3-(pyridin-4-yl)-6,7-dihydropyrazolo[1,5-a]pyrazine-5(4H)-carboxylate (170 mg, 53% purity, 205 μmol, Intermediate 399) in hydrochloric acid (4M in dioxane, 2.0 ml) was stirred at 25° C. for 3 hours. The reaction mixture was concentrated under reduced pressure to give rac-(RS)-6-methyl-2-(naphthalen-2-yl)-3-(pyridin-4-yl)-4,5,6,7-tetrahydropyrazolo[1,5-a]pyrazine (80.0 mg, 92% purity, 95% yield) as a colorless oil.

LC-MS (Method 16): Rₜ=0.684 min; MS (ESIpos): m/z=341.2 [M+H]⁺.

### Intermediate 401

### 6-bromo-2-chloro-1-benzothiophene

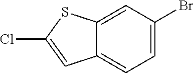 [SMILES: Clc1cc2ccc(Br)cc2s1]

To a solution of 6-bromo-1-benzothiophene (1.00 g, 4.69 mmol) in tetrahydrofuran (15 ml) was added dropwise lithium diisopropylamide (2 M in hexane and tetrahydrofuran, 2.6 ml, 5.20 mmol) at −70° C. After stirring at −70° C. for 0.5 hour under nitrogen atmosphere, to the solution above was added N-chlorosuccinimide (940 mg, 7.04 mmol) at −70° C. and the mixture was stirred at −20° C. for 16 hours. The reaction mixture was diluted with water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=1:0) to give 6-bromo-2-chloro-1-benzothiophene (220 mg, 19% yield) as a colorless oil.

¹H-NMR (400 MHz, CDCl₃), δ [ppm]=7.85 (d, J=1.6 Hz, 1H), 7.55-7.50 (m, 1H), 7.55-7.49 (m, 1H), 7.48-7.44 (m, 1H), 7.15 (s, 1H).

### Intermediate 402

### 2-(2-chloro-1-benzothiophen-6-yl)-4,4,5,5-tetramethyl-1,3,2-dioxaborolane

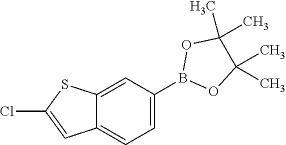 [SMILES: CC1(C)OB(c2ccc3cc(Cl)sc3c2)OC1(C)C]

To a solution of 6-bromo-2-chloro-1-benzothiophene (220 mg, 889 μmol, Intermediate 401) and 4,4,4′,4′,5,5,5′,5′-octamethyl-2,2′-bi-1,3,2-dioxaborolane (248 mg, 978 μmol) in 1,4-dioxane (5.0 ml) were added potassium acetate (174 mg, 1.78 mmol) and 1,1′-bis(diphenylphosphino) ferrocene]dichloropalladium (II) (74.0 mg, 72.5 μmol) at 25° C. The mixture was stirred at 80° C. for 16 hours under nitrogen atmosphere. The reaction mixture was filtered and the filtrate was concentrated in vacuo to give a residue. The residue was purified by column chromatography on silica gel (300~400 mesh, petroleum ether:ethyl acetate=100:1 to 10:1) to give 2-(2-chloro-1-benzothiophen-6-yl)-4,4,5,5-tetramethyl-1,3,2-dioxaborolane (290 mg, 80% purity, 89% yield) as a yellow oil.

¹H-NMR (400 MHz, CDCl₃), δ [ppm]=8.19 (s, 1H), 7.76 (d, J=8.0 Hz, 1H), 7.66 (d, J=8.0 Hz, 1H), 7.19 (s, 1H), 1.37 (s, 12H).

### Intermediate 403

### rac-tert-butyl (RS)-2-(2-chloro-1-benzothiophen-6-yl)-6-methyl-3-(pyridin-4-yl)-6,7-dihydropyrazolo[1,5-a]pyrazine-5(4H)-carboxylate

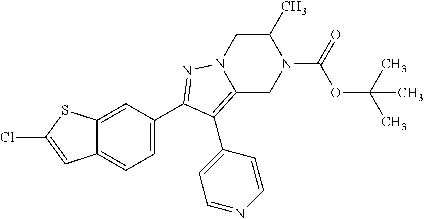 [SMILES: CC1Cn2nc(-c3ccc4cc(Cl)sc4c3)c(-c3ccncc3)c2CN1C(=O)OC(C)(C)C]

To a solution of rac-tert-butyl (6RS)-2-bromo-6-methyl-3-(pyridin-4-yl)-6,7-dihydropyrazolo[1,5-a]pyrazine-5(4H)-carboxylate (150 mg, 95% purity, 362 μmol, Intermediate 383) and 2-(2-chloro-1-benzothiophen-6-yl)-4,4,5,5-tetramethyl-1,3,2-dioxaborolane (254 mg, 80% purity, 688 μmol, Intermediate 402) in 1,4-dioxane (10 ml) and water (2.0 ml) were added sodium carbonate (76.8 mg, 725 μmol) and 1,1′-bis(diphenylphosphino) ferrocene]dichloropalladium (II) (26.5 mg, 36.2 μmol) at 25° C. The mixture was stirred at 100° C. for 16 hours under nitrogen atmosphere. The reaction mixture was filtered through a pad of celite and the filtrate was concentrated in vacuo to give a residue. The residue was purified by column chromatography on silica gel (300~400 mesh, petroleum ether:ethyl acetate=10:1 to 3:1) to give rac-tert-butyl (RS)-2-(2-chloro-1-benzothiophen-6-yl)-6-methyl-3-(pyridin-4-yl)-6,7-dihydropyrazolo[1,5-a]pyrazine-5(4H)-carboxylate (140 mg, 80% yield) as a yellow oil.

LC-MS (Method 14): Rₜ=0.904 minute; MS (ESIpos): m/z=481.2 [M+H]⁺.

### Intermediate 404

### rac-(RS)-2-(2-chloro-1-benzothiophen-6-yl)-6-methyl-3-(pyridin-4-yl)-4,5,6,7-tetrahydropyrazolo[1,5-a]pyrazine hydrogen chloride (1/1)

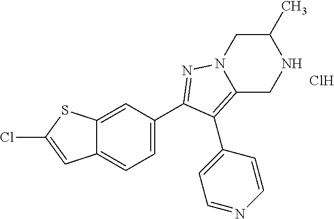 [SMILES: CC1Cn2nc(-c3ccc4cc(Cl)sc4c3)c(-c3ccncc3)c2CN1 ; Cl]

To a solution of rac-tert-butyl (6RS)-2-(2-chloro-1-benzothiophen-6-yl)-6-methyl-3-(pyridin-4-yl)-6,7-dihydropyrazolo[1,5-a]pyrazine-5(4H)-carboxylate (140 mg, 291 μmol, Intermediate 403) in 1,4-dioxane (2.0 ml) was added hydrochloric acid (4M in dioxane, 2.0 ml, 8.0 mmol) at 25° C. The mixture was stirred at 25° C. for 2 hours. The reaction mixture was concentrated in vacuo to give rac-(RS)-2-(2-chloro-1-benzothiophen-6-yl)-6-methyl-3-(pyridin-4-yl)-4,5,6,7-tetrahydropyrazolo[1,5-a]pyrazine hydrochloride (110 mg, 91% yield) as a yellow solid.

LC-MS (Method 14): Rₜ=0.737 minute; MS (ESIpos): m/z=381.1 [M+H]⁺.

### Intermediate 405

### 2-(1-benzothiophen-6-yl)-4,4,5,5-tetramethyl-1,3,2-dioxaborolane

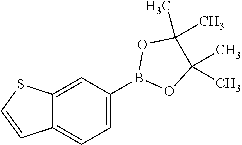 [SMILES: CC1(C)OB(c2ccc3ccsc3c2)OC1(C)C]

To a solution of 6-bromo-1-benzothiophene (1.00 g, 4.69 mmol) in 1,4-dioxane (20 ml) were added 4,4,4′,4′,5,5,5′,5′-octamethyl-2,2′-bi-1,3,2-dioxaborolane (1.79 g, 7.04 mmol), potassium acetate (921 mg, 9.39 mmol) and [1,1′-bis(diphenylphosphino) ferrocene]palladium (II) dichloride dichloromethane complex (343 mg, 469 μmol) at 25° C. The mixture was stirred at 90° C. for 16 hours under nitrogen atmosphere. The reaction mixture was filtered through a pad of celite and the filtrate was concentrated in vacuo to give a residue. The residue was purified by column chromatography on silica gel (100~200 mesh, petroleum ether:ethyl acetate=99:1) to give 2-(1-benzothiophen-6-yl)-4,4,5,5-tetramethyl-1,3,2-dioxaborolane (2.30 g, 50% purity, 94% yield) as a brown solid.

¹H NMR (400 MHz, DMSO-d6), δ [ppm]=8.38 (s, 1H), 7.86-7.81 (m, 1H), 7.80-7.73 (m, 1H), 7.53 (d, J=5.6 Hz, 1H), 7.37-7.32 (m, 1H), 1.38 (s, 12H).

### Intermediate 406

### rac-tert-butyl (RS)-2-(1-benzothiophen-6-yl)-6-methyl-3-(pyridin-4-yl)-6,7-dihydropyrazolo[1,5-a]pyrazine-5(4H)-carboxylate

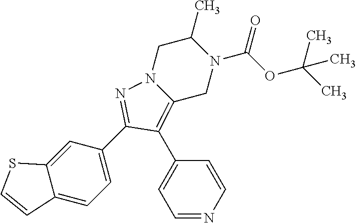 [SMILES: CC1Cn2nc(-c3ccc4ccsc4c3)c(-c3ccncc3)c2CN1C(=O)OC(C)(C)C]

To a solution of rac-tert-butyl (RS)-2-bromo-6-methyl-3-(pyridin-4-yl)-6,7-dihydropyrazolo[1,5-a]pyrazine-5(4H)-carboxylate (300 mg, 95% purity, 725 μmol, Intermediate 383) in 1,4-dioxane (4.5 ml) and water (1.5 ml) were added 2-(1-benzothiophen-6-yl)-4,4,5,5-tetramethyl-1,3,2-dioxaborolane (943 mg, 50% purity, 1.81 mmol, Intermediate 405), sodium carbonate (230 mg, 2.17 mmol) and [1,1′-Bis(diphenylphosphino) ferrocene]palladium (II) dichloride dichloromethane complex (53.0 mg, 72.5 μmol) at 25° C. The mixture was stirred at 90° C. for 16 hours under nitrogen atmosphere. The reaction mixture was filtered through a pad of celite and the filtrate was concentrated in vacuo to give a residue. The residue was purified by column chromatography on silica gel (100~200 mesh, petroleum ether:ethyl acetate=1:1) to give rac-tert-butyl (RS)-2-(1-benzothiophen-6-yl)-6-methyl-3-(pyridin-4-yl)-6,7-dihydropyrazolo[1,5-a]pyrazine-5(4H)-carboxylate (200 mg, 98% purity, 61% yield) as a yellow oil.

LC-MS (Method 16): Rₜ=0.823 min; MS (ESIpos): m/z=447.1 [M+H]⁺.

### Intermediate 407

### rac-(RS)-2-(1-benzothiophen-6-yl)-6-methyl-3-(pyridin-4-yl)-4,5,6,7-tetrahydropyrazolo[1,5-a]pyrazine hydrogen chloride (1/1)

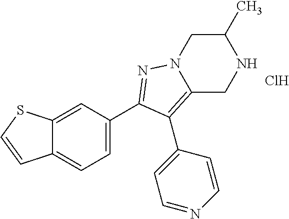 [SMILES: CC1Cn2nc(-c3ccc4ccsc4c3)c(-c3ccncc3)c2CN1 ; Cl]

The mixture of rac-tert-butyl (RS)-2-(1-benzothiophen-6-yl)-6-methyl-3-(pyridin-4-yl)-6,7-dihydropyrazolo[1,5-a]pyrazine-5(4H)-carboxylate (260 mg, 98% purity, 571 μmol, Intermediate 406) in hydrochloric acid (4M in ethyl acetate, 5.0 ml) was stirred at 25° C. for 2.5 hours. The reaction mixture was concentrated in vacuo to give rac-(RS)-2-(1-benzothiophen-6-yl)-6-methyl-3-(pyridin-4-yl)-4,5,6,7-tetrahydropyrazolo[1,5-a]pyrazine hydrochloride (1:1) (200 mg, 97% purity, 89% yield) as a yellow solid.

LC-MS (Method 16): Rₜ=0.463 min; MS (ESIpos): m/z=347.2 [M+H]⁺.

### Intermediate 408

### methyl 3-bromo-1-{2-[(tert-butoxycarbonyl)amino]ethyl}-1H-pyrazole-5-carboxylate

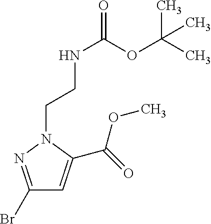 [SMILES: COC(=O)c1cc(Br)nn1CCNC(=O)OC(C)(C)C]

To a solution of methyl 3-bromo-1H-pyrazole-5-carboxylate (5.00 g, 24.4 mmol), tert-butyl (2-hydroxyethyl) carbamate (5.90 g, 36.6 mmol) and triphenylphosphine (12.8 g, 48.8 mmol) in tetrahydrofuran (50 ml) was added diisopropyl azodicarboxylate (8.6 ml, 49.0 mmol) at 0° C. After stirring at 25° C. for 4 hours, the mixture was concentrated under reduced pressure to give a residue. The residue was purified by column chromatography on silica gel (100~200 mesh, petroleum ether:ethyl acetate=1:0 to 3:1) to give methyl 3-bromo-1-{2-[(tert-butoxycarbonyl)amino]ethyl}-1H-pyrazole-5-carboxylate (6.94 g, 20.0 mmol, 97% purity, 82% yield) as a colorless oil.

LC-MS (Method 14): Rₜ=0.419 minute; MS (ESIpos): m/z=370.0 [M+Na]⁺.

### Intermediate 409

### 3-bromo-1-{2-[(tert-butoxycarbonyl)amino]ethyl}-1H-pyrazole-5-carboxylic acid

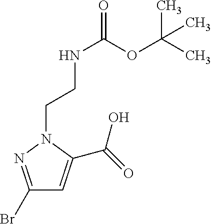 [SMILES: CC(C)(C)OC(=O)NCCn1nc(Br)cc1C(=O)O]

To a solution of methyl 3-bromo-1-{2-[(tert-butoxycarbonyl)amino]ethyl}-1H-pyrazole-5-carboxylate (6.20 g, 97% purity, 17.3 mmol, Intermediate 407) in ethanol (60 ml) and water (20 ml) was added potassium hydroxide (1.94 g, 34.5 mmol) at 25° C. and the mixture was stirred at 25° C. for 1 hour. The reaction mixture was concentrated under reduced pressure to give a residue, and pH of the residue was adjusted to 5 with saturated hydrochloride aqueous solution (1 M). The resulting solution was extracted with ethyl acetate. The organic phase was washed with brine, dried over anhydrous sodium sulfate, filtered and concentrated under reduced pressure to give 3-bromo-1-(2-((tert-butoxycarbonyl)amino)ethyl)-1H-pyrazole-5-carboxylic acid (5.20 g, 98% purity, 21.8 mmol, 88% yield) as a brown oil.

LC-MS (Method 14): Rₜ=0.368 minute; MS (ESIpos): m/z=357.8 [M+Na]⁺

### Intermediate 410

### tert-butyl(2-{3-bromo-5-[methoxy(methyl) carbamoyl]-1H-pyrazol-1-yl}ethyl) carbamate

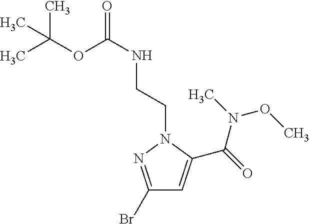 [SMILES: CON(C)C(=O)c1cc(Br)nn1CCNC(=O)OC(C)(C)C]

To a solution of-(2-aminoethyl)-3-bromo-1H-pyrazole-5-carboxylic acid (5.20 g, 98% purity, 21.8 mmol, Intermediate 409) and N-methoxymethanamine hydrogen chloride (1/1) (2.55 g. 26.1 mmol) in N, N-dimethylformamide (50 ml) were added (1-[bis(dimethylamino)methylene]-1H-1,2,3-triazolo[4,5-b]pyridinium 3-oxid hexafluorophosphate (12.4 g, 32.7 mmol) and N,N-diisopropylethylamine (7.6 ml, 44.0 mmol) at 25° C. The mixture was stirred at 25° C. for 2 hours. The mixture was diluted with water and extracted with ethyl acetate. The combined organic phase was washed with brine, dried over anhydrous sodium sulfate, filtered and concentrated under reduced pressure to give a residue. The residue above was purified by column chromatography on silica gel (100~200 mesh, petroleum ether:ethyl acetate=10:1 to 3:1) to give tert-butyl (2-(3-bromo-5-(methoxy(methyl) carbamoyl)-1H-pyrazol-1-yl)ethyl) carbamate (6.20 g, 98% purity, 74% yield) as a colorless oil.

LC-MS (Method 14): Rₜ=0.375 minute; MS (ESIpos): m/z=400.9 [M+Na]⁺.

¹H-NMR (400 MHz, CDCl₃), δ [ppm]=6.75 (s, 1H), 5.08 (s, 1H), 5.45 (t, J=5.2 Hz, 2H), 3.67 (s, 3H), 3.72-3.53 (m, 2H), 3.41 (s, 3H), 1.40 (s, 9H).

### Intermediate 411

### tert-butyl [2-(5-acetyl-3-bromo-1H-pyrazol-1-yl)ethyl]carbamate

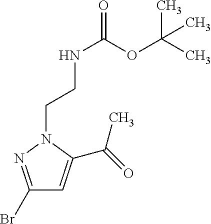 [SMILES: CC(=O)c1cc(Br)nn1CCNC(=O)OC(C)(C)C]

To a solution of tert-butyl(2-{3-bromo-5-[methoxy(methyl) carbamoyl]-1H-pyrazol-1-yl}ethyl) carbamate (5.20 g, 98% purity, 13.5 mmol, Intermediate 410) in tetrahydrofuran (100 ml) was added bromo(methyl) magnesium (3M in diethyl ether, 23 ml, 68.0 mmol) at 0° C. under nitrogen atmosphere. The mixture was stirred at 25° C. for 2 hours. The reaction was quenched with saturated ammonium chloride aqueous solution and extracted with ethyl acetate. The combined organic phase was washed with brine, dried over anhydrous sodium sulfate, filtered and concentrated under reduced pressure to give tert-butyl(2-(5-acetyl-3-bromo-1H-pyrazol-1-yl)ethyl) carbamate (4.50 g, 92% purity, 92% yield) as a colorless oil.

LC-MS (Method 14): Rₜ=0.890 minute; MS (ESIpos): m/z=277.1 [M−55]⁺.

¹H-NMR (400 MHz, CDCl₃), δ [ppm]=6.83 (s, 1H), 4.62 (t, J=5.6 Hz, 2H), 3.59-3.51 (m, 2H), 2.51 (s, 3H), 1.41 (s, 9H).

### Intermediate 412

### 2-bromo-4-methyl-6,7-dihydropyrazolo[1,5-a]pyrazine

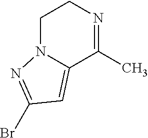

To a solution of tert-butyl [2-(5-acetyl-3-bromo-1H-pyrazol-1-yl)ethyl]carbamate (4.50 g, 92% purity, 12.5 mmol, Intermediate 411) in dichloromethane (20 ml) was added trifluoroacetic acid (10 ml, 130 mmol) and the mixture was stirred at 25° C. for 16 hours. The mixture was concentrated under reduced pressure to give a residue. pH of the residue was adjusted to 8 with saturated sodium bicarbonate aqueous solution and the resulting solution was extracted with ethyl acetate. The combined organic phase was washed with brine, dried over anhydrous sodium sulfate, filtered and concentrated under reduced pressure to give 2-bromo-4-methyl-6,7-dihydropyrazolo[1,5-a]pyrazine (2.35 g, 88% yield) as a yellow oil.

¹H-NMR (400 MHz, CDCl₃), δ [ppm]=6.43 (s, 1H), 4.15-4.09 (m, 2H), 4.04-3.97 (m, 2H), 2.32 (s, 3H).

### Intermediate 413

### rac-(RS)-2-bromo-4-methyl-4,5,6,7-tetrahydropyrazolo[1,5-a]pyrazine

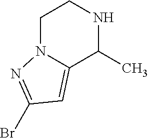

To a solution of 2-bromo-4-methyl-6,7-dihydropyrazolo[1,5-a]pyrazine (2.35 g, 11.0 mmol, Intermediate 412) in methanol (40 ml) was added sodium cyanoborohydride (1.38 g, 22.0 mmol) at 0° C. and the mixture was stirred at 25° C. for 1 hour. The reaction mixture was concentrated under reduced pressure to give a residue. The residue was diluted with water and extracted with ethyl acetate. The combined organic phase was washed with brine, dried over anhydrous sodium sulfate, filtered and concentrated in vacuo to give rac-(RS)-2-bromo-4-methyl-4,5,6,7-tetrahydropyrazolo[1,5-a]pyrazine (2.28 g, 75% purity, 72% yield) as a yellow oil.

LC-MS (Method 15): Rₜ=0.436 minute; MS (ESIpos): m/z=215.6 [M+H]⁺.

### Intermediate 414

### rac-tert-butyl (RS)-2-bromo-4-methyl-6,7-dihydropyrazolo[1,5-a]pyrazine-5(4H)-carboxylate

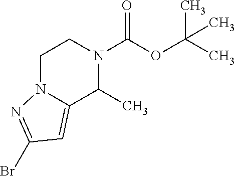 [SMILES: CC1c2cc(Br)nn2CCN1C(=O)OC(C)(C)C]

To a solution of rac-(RS)-2-bromo-4-methyl-4,5,6,7-tetrahydropyrazolo[1,5-a]pyrazine (2.28 g, 75% purity, 7.91 mmol, Intermediate 413) in dichloromethane (30 ml) were added di-tert-butyl dicarbonate (2.0 ml, 8.7 mmol) and triethylamine (1.7 ml) at 25° C. and the mixture was stirred at 25° C. for 2 hours. The reaction mixture was concentrated under reduced pressure to give a residue. The residue above was purified by column chromatography on silica gel (100~200 mesh, petroleum ether:ethyl acetate=10:1 to 3:1) to give rac-tert-butyl (RS)-2-bromo-4-methyl-6,7-dihydropyrazolo[1,5-a]pyrazine-5(4H)-carboxylate (1.72 g, 69% yield) as a colourless oil.

LC-MS (Method 15): Rₜ=1.165 minute; MS (ESIpos): m/z=318.1 [M+H]⁺.

¹H-NMR (400 MHz, CDCl₃), δ [ppm]=6.07 (s, 1H), 5.31 (s, 1H), 5.16 (s, 1H), 4.18-4.11 (m, 1H), 4.04-3.97 (m, 1H), 3.52-3.21 (m, 1H), 1.50 (s, 9H), 1.43 (d, J=7.2 Hz, 3H).

### Intermediate 415

### rac-tert-butyl (RS)-2-(4-fluorophenyl)-4-methyl-6,7-dihydropyrazolo[1,5-a]pyrazine-5(4H)-carboxylate

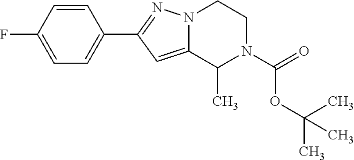 [SMILES: CC1c2cc(-c3ccc(F)cc3)nn2CCN1C(=O)OC(C)(C)C]

To a solution of rac-tert-butyl (RS)-2-bromo-4-methyl-6,7-dihydropyrazolo[1,5-a]pyrazine-5(4H)-carboxylate (330 mg, 1.05 mmol, Intermediate 414) and (4-fluorophenyl) boronic acid (186 mg, 1.33 mmol) in 1,4-dioxane (10 ml) and water (2 ml) were added 1′-bis(diphenylphosphino) ferrocene]dichloropalladium (II) (64.8 mg, 0.0890 mmol) and sodium carbonate (188 mg, 1.77 mmol) at 20° C. The mixture was stirred at 80° C. for 16 hours under nitrogen atmosphere. The mixture was filtered and the filtrate was concentrated in vacuo to give a residue. The residue was purified by column chromatography on silica gel (300~400 mesh, petroleum ether:ethyl acetate=10:1 to 5:1) to give rac-tert-butyl (RS)-2-(4-fluorophenyl)-4-methyl-6,7-dihydropyrazolo[1,5-a]pyrazine-5(4H)-carboxylate (320 mg, 92% yield) as a yellow oil.

LC-MS (Method 14): Rₜ=1.010 minute; MS (ESIpos): m/z=332.2 [M+H]⁺.

### Intermediate 416

### rac-tert-butyl (RS)-2-(4-fluoro-3-iodophenyl)-4-methyl-6,7-dihydropyrazolo[1,5-a]pyrazine-5(4H)-carboxylate

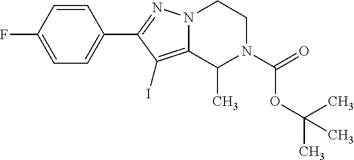 [SMILES: CC1c2c(I)c(-c3ccc(F)cc3)nn2CCN1C(=O)OC(C)(C)C]

To a solution of rac-tert-butyl (RS)-2-(4-fluorophenyl)-4-methyl-6,7-dihydropyrazolo[1,5-a]pyrazine-5(4H)-carboxylate (320 mg, 0.967 mmol, Intermediate 415) in dichloromethane (10 ml) was added N-iodosuccinimide (380 mg, 1.69 mmol) at 0° C. and the mixture was stirred at 25° C. for 16 hours. The reaction mixture was poured into saturated sodium thiosulfate solution and saturated sodium bicarbonate solution, and extracted with dichloromethane. The combined organic phase was washed with brine, dried over anhydrous sodium sulfate, filtered and concentrated in vacuo to give a residue. The residue was purified by column chromatography on silica gel (100-200 mesh, petroleum ether:ethyl acetate=10:1 to 5:1) to give rac-tert-butyl (RS)-2-(4-fluoro-3-iodophenyl)-4-methyl-6,7-dihydropyrazolo[1,5-a]pyrazine-5(4H)-carboxylate (440 mg, 99% yield) as a colorless oil.

LC-MS (Method 14): Rₜ=1.090 minute; MS (ESIpos): m/z=458.1 [M+H]⁺.

### Intermediate 417

### rac-tert-butyl (RS)-2-(4-fluorophenyl)-4-methyl-3-(pyridin-4-yl)-6,7-dihydropyrazolo[1,5-a]pyrazine-5(4H)-carboxylate

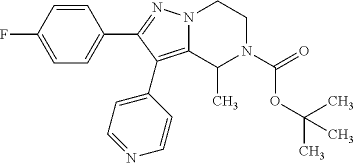 [SMILES: CC1c2c(-c3ccncc3)c(-c3ccc(F)cc3)nn2CCN1C(=O)OC(C)(C)C]

To a solution of rac-tert-butyl (RS)-2-(4-fluoro-3-iodophenyl)-4-methyl-6,7-dihydropyrazolo[1,5-a]pyrazine-5(4H)-carboxylate (170 mg, 0.372 mmol, Intermediate 416) and pyridin-4-ylboronic acid (54.8 mg, 0.446 mmol) in 1,4-dioxane (5 ml) and water (1 ml) were added sodium carbonate (78.8 mg, 0.744 mmol) and 1,1′-bis(diphenylphosphino) ferrocene]dichloropalladium (II) (27.0 mg, 0.0370 mmol) at 25° C. The mixture was stirred at 100° C. for 16 hours under nitrogen atmosphere. The reaction mixture was filtered and the filtrate was concentrated in vacuo to give a residue. The residue was purified by column chromatography on silica gel (300~400 mesh, petroleum ether:ethyl acetate=10:1 to 3:1) to give rac-tert-butyl (R)-2-bromo-4-methyl-3-(pyridin-4-yl)-6,7-dihydropyrazolo[1,5-a]pyrazine-5(4H)-carboxylate (140 mg, 92% yield) as a yellow solid.

LC-MS (Method 14): Rₜ=0.837 minute; MS (ESIpos): m/z=409.3 [M+H]⁺.

### Intermediate 418

### rac-(RS)-2-(4-fluorophenyl)-4-methyl-3-(pyridin-4-yl)-4,5,6,7-tetrahydropyrazolo[1,5-a]pyrazine hydrogen chloride (1/1)

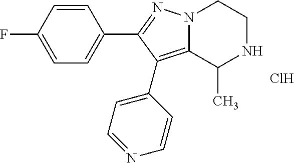 [SMILES: CC1NCCn2nc(-c3ccc(F)cc3)c(-c3ccncc3)c21 ; Cl]

To a solution of rac-tert-butyl (RS)-2-(4-fluorophenyl)-4-methyl-3-(pyridin-4-yl)-6,7-dihydropyrazolo[1,5-a]pyrazine-5(4H)-carboxylate (140 mg, 0.343 mmol, Intermediate 417) in 1,4-dioxane (2 ml) was added hydrochloric acid (4M in 1,4-dioxane, 2 ml) at 25° C. After stirring at 25° C. for 2 hours, the reaction mixture was concentrated in vacuo to give rac-(RS)-2-(4-fluorophenyl)-4-methyl-3-(pyridin-4-yl)-4,5,6,7-tetrahydropyrazolo[1,5-a]pyrazine hydrochloride (1:1) (118 mg, 100% yield) as a yellow solid.

### Intermediate 419

### rac-tert-butyl (RS)-2-bromo-3-iodo-4-methyl-6,7-dihydropyrazolo[1,5-a]pyrazine-5(4H)-carboxylate

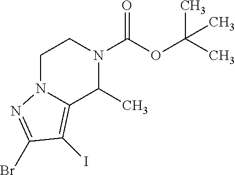 [SMILES: CC1c2c(I)c(Br)nn2CCN1C(=O)OC(C)(C)C]

To a solution of rac-tert-butyl (RS)-2-bromo-4-methyl-6,7-dihydropyrazolo[1,5-a]pyrazine-5(4H)-carboxylate (1.10 g, 3.48 mmol, Intermediate 414) in dichloromethane (30 ml) was added N-iodosuccinimide (2.35 g, 10.4 mmol) at 0° C. and the mixture was stirred at 25° C. for 16 hours. The reaction mixture was poured into saturated sodium thiosulfate solution and saturated sodium bicarbonate solution, and extracted with dichloromethane. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 3:1) to give rac-tert-butyl (RS)-2-bromo-3-iodo-4-methyl-6,7-dihydropyrazolo[1,5-a]pyrazine-5(4H)-carboxylate (1.45 g, 94% yield) as a colorless oil.

LC-MS (Method 14): R₁=0.949 minute; MS (ESIpos): m/z=441.9 [M+H]⁺.

### Intermediate 420

### rac-tert-butyl (RS)-2-bromo-4-methyl-3-(pyridin-4-yl)-6,7-dihydropyrazolo[1,5-a]pyrazine-5(4H)-carboxylate

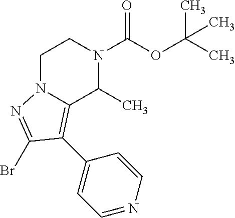 [SMILES: CC1c2c(-c3ccncc3)c(Br)nn2CCN1C(=O)OC(C)(C)C]

To a solution of tert-butyl (RS)-2-bromo-3-iodo-4-methyl-6,7-dihydropyrazolo[1,5-a]pyrazine-5(4H)-carboxylate (1.45 g, 3.28 mmol, Intermediate 419) and pyridin-4-ylboronic acid (484 mg, 3.94 mmol) in 1,4-dioxane (50 ml) and water (10 ml) were added sodium carbonate (695 mg, 6.56 mmol) and 1,1′-bis(diphenylphosphino) ferrocene]dichloropalladium (II) (240 mg, 0.328 mmol) at 25° C. The mixture was stirred at 80° C. for 16 hours under nitrogen atmosphere. The reaction mixture was filtered and the filtrate was concentrated in vacuo to give a residue. The residue was purified by column chromatography on silica gel (300~400 mesh, petroleum ether:ethyl acetate=10:1 to 3:1) to give rac-tert-butyl (RS)-2-bromo-4-methyl-3-(pyridin-4-yl)-6,7-dihydropyrazolo[1,5-a]pyrazine-5(4H)-carboxylate (1.00 g, 78% yield) as a yellow solid.

LC-MS (Method 14): Rₜ=0.793 minute; MS (ESIpos): m/z=393.1 [M+H]⁺

### Intermediate 421

### rac-tert-butyl (RS)-2-(4-chloro-2-fluorophenyl)-4-methyl-3-(pyridin-4-yl)-6,7-dihydropyrazolo[1,5-a]pyrazine-5(4H)-carboxylate

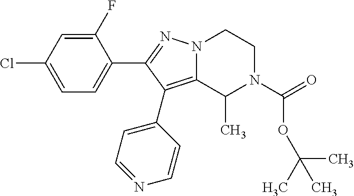 [SMILES: CC1c2c(-c3ccncc3)c(-c3ccc(Cl)cc3F)nn2CCN1C(=O)OC(C)(C)C]

To a solution of rac-tert-butyl (RS)-2-bromo-4-methyl-3-(pyridin-4-yl)-6,7-dihydropyrazolo[1,5-a]pyrazine-5(4H)-carboxylate (300 mg, 0.763 mmol, Intermediate 420) and (4-chloro-2-fluorophenyl) boronic acid (200 mg, 1.14 mmol) in 1,4-dioxane (10 ml) and water (2 ml) were added sodium carbonate (162 mg, 1.53 mmol) and 1,1′-bis(diphenylphosphino) ferrocene]dichloropalladium (II) (55.8 mg, 0.076 mmol) at 25° C. The mixture was stirred at 100° C. for 16 hours under nitrogen atmosphere. The reaction mixture was filtered and the filtrate was concentrated in vacuo to give a residue. The residue was purified by column chromatography on silica gel (300~400 mesh, petroleum ether:ethyl acetate=5:1 to 1:1) to give rac-tert-butyl (RS)-2-(4-chloro-2-fluorophenyl)-4-methyl-3-(pyridin-4-yl)-6,7-dihydropyrazolo[1,5-a]pyrazine-5(4H)-carboxylate (290 mg, 86% yield) as a yellow solid.

LC-MS (Method 14): Rₜ=0.864 minute; MS (ESIpos): m/z=443.2 [M+H]⁺.

### Intermediate 422

### rac-(RS)-2-(4-chloro-2-fluorophenyl)-4-methyl-3-(pyridin-4-yl)-4,5,6,7-tetrahydropyrazolo[1,5-a]pyrazine hydrogen chloride (1/1)

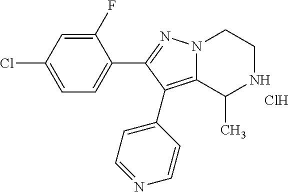 [SMILES: CC1NCCn2nc(-c3ccc(Cl)cc3F)c(-c3ccncc3)c21 ; Cl]

To a solution of rac-tert-butyl (RS)-2-(4-chloro-2-fluorophenyl)-4-methyl-3-(pyridin-4-yl)-6,7-dihydropyrazolo[1,5-a]pyrazine-5(4H)-carboxylate (190 mg, 0.429 mmol, Intermediate 421) in 1,4-dioxane (2 ml) was added hydrochloric acid (4M in dioxane, 2 ml) at 25° C. After stirring at at 25° C. for 2 hours, the reaction mixture was concentrated in vacuo to give rac-(RS)-2-(4-chloro-2-fluorophenyl)-4-methyl-3-(pyridin-4-yl)-4,5,6,7-tetrahydropyrazolo[1,5-a]pyrazine hydrochloride (1:1) (160 mg, 98% yield) as a yellow solid.

LC-MS (Method 16): Rₜ=0.865 minute; MS (ESIpos): m/z=343.1 [M+H]⁺.

### Intermediate 423

### rac-tert-butyl (RS)-2-(4-methoxyphenyl)-4-methyl-3-(pyridin-4-yl)-6,7-dihydropyrazolo[1,5-a]pyrazine-5(4H)-carboxylate

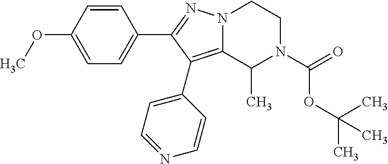 [SMILES: COc1ccc(-c2nn3c(c2-c2ccncc2)C(C)N(C(=O)OC(C)(C)C)CC3)cc1]

To a solution of rac-tert-butyl (RS)-2-bromo-4-methyl-3-(pyridin-4-yl)-6,7-dihydropyrazolo[1,5-a]pyrazine-5(4H)-carboxylate (200 mg, 0.509 mmol, Intermediate 420) and (4-methoxyphenyl) boronic acid (92.7 mg, 0.610 mmol) in 1,4-dioxane (5 ml) and water (1 ml) were added sodium carbonate (108 mg, 1.02 mmol) and 1,1′-bis(diphenylphosphino) ferrocene]dichloropalladium (II) (37.2 mg, 0.051 mmol) at 25° C. The mixture was stirred at 80° C. for 16 hours under nitrogen atmosphere. The reaction mixture was filtered and the filtrate was concentrated in vacuo to give a residue. The residue was purified by column chromatography on silica gel (300~400 mesh, petroleum ether:ethyl acetate=5:1 to 1:1) to give rac-tert-butyl (RS)-2-(4-methoxyphenyl)-4-methyl-3-(pyridin-4-yl)-6,7-dihydropyrazolo[1,5-a]pyrazine-5(4H)-carboxylate (210 mg, 98% yield) as a yellow solid.

LC-MS (Method 14): Rₜ=0.824 minute; MS (ESIpos): m/z=421.3 [M+H]⁺.

### Intermediate 424

### rac-(RS)-2-(4-methoxyphenyl)-4-methyl-3-(pyridin-4-yl)-4,5,6,7-tetrahydropyrazolo[1,5-a]pyrazine hydrogen chloride (1/1)

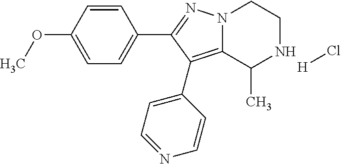

The mixture of rac-tert-butyl (RS)-2-(4-methoxyphenyl)-4-methyl-3-(pyridin-4-yl)-6,7-dihydropyrazolo[1,5-a]pyrazine-5(4H)-carboxylate (210 mg, 0.499 mmol, Intermediate 423) in hydrochloric acid (4M in dioxane, 5 ml) was stirred at 25° C. for 3 hours. The reaction mixture was concentrated in vacuo to give rac-(RS)-2-(4-methoxyphenyl)-4-methyl-3-(pyridin-4-yl)-4,5,6,7-tetrahydropyrazolo[1,5-a]pyrazine hydrogen chloride (1:1) (178 mg, 100% yield) as a white solid.

LC-MS (Method 15): Rₜ=0.669 minute; MS (ESIpos): m/z=321.2 [M+H]⁺.

### Intermediate 425

### rac-tert-butyl (RS)-2-(1-benzothiophen-5-yl)-4-methyl-3-(pyridin-4-yl)-6,7-dihydropyrazolo[1,5-a]pyrazine-5(4H)-carboxylate

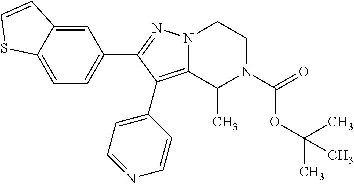 [SMILES: CC1c2c(-c3ccncc3)c(-c3ccc4sccc4c3)nn2CCN1C(=O)OC(C)(C)C]

To a solution of rac-tert-butyl (RS)-2-bromo-4-methyl-3-(pyridin-4-yl)-6,7-dihydropyrazolo[1,5-a]pyrazine-5(4H)-carboxylate (150 mg, 0.381 mmol, Intermediate 420) in 1,4-dioxane (3 ml) and water (1 ml) were added 2-(1-benzothiophen-5-yl)-4,4,5,5-tetramethyl-1,3,2-dioxaborolane (149 mg, 0.572 mmol), sodium carbonate (121 mg, 1.14 mmol) and 1,1′-bis(diphenylphosphino) ferrocene]dichloropalladium (II) (27.9 mg, 0.038 mmol) at 25° C. The mixture was stirred at 100° C. for 16 hours under nitrogen atmosphere. The mixture was filtered through a pad of celite and the filtrate was concentrated in vacuo to give a residue. The residue was purified by column chromatography on silica gel (100~200 mesh, petroleum ether:ethyl acetate=2:1) to give rac-tert-butyl (RS)-2-(1-benzothiophen-5-yl)-4-methyl-3-(pyridin-4-yl)-6,7-dihydropyrazolo[1,5-a]pyrazine-5(4H)-carboxylate (200 mg, 79% purity, 93% yield) as a brown oil.

LC-MS (Method 14): Rₜ=0.556 minute; MS (ESIpos): m/z=447.3 [M+H]⁺.

### Intermediate 426

### rac-(RS)-2-(1-benzothiophen-5-yl)-4-methyl-3-(pyridin-4-yl)-4,5,6,7-tetrahydropyrazolo[1,5-a]pyrazine hydrogen chloride (1/1)

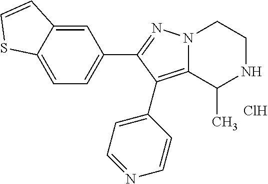 [SMILES: CC1NCCn2nc(-c3ccc4sccc4c3)c(-c3ccncc3)c21 ; Cl]

The mixture of rac-tert-butyl (RS)-2-(1-benzothiophen-5-yl)-4-methyl-3-(pyridin-4-yl)-6,7-dihydropyrazolo[1,5-a]pyrazine-5(4H)-carboxylate (200 mg, 79% purity, 0.354 mmol, Intermediate 425) in hydrochloric acid (4M in ethyl acetate, 5 ml) was stirred at 25° C. for 2.5 hours. The reaction mixture was concentrated in vacuo to give rac-(RS)-2-(1-benzothiophen-5-yl)-4-methyl-3-(pyridin-4-yl)-4,5,6,7-tetrahydropyrazolo[1,5-a]pyrazine hydrochloride (1:1) (130 mg, 84% purity, 81% yield) as a yellow solid.

LC-MS (Method 14): Rₜ=0.453 minute; MS (ESIpos): m/z=347.3 [M+H]⁺.

### Intermediate 427

### rac-tert-butyl (RS)-2-(1H-indol-6-yl)-4-methyl-3-(pyridin-4-yl)-6,7-dihydropyrazolo[1,5-a]pyrazine-5(4H)-carboxylate

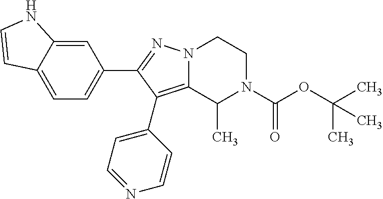 [SMILES: CC1c2c(-c3ccncc3)c(-c3ccc4cc[nH]c4c3)nn2CCN1C(=O)OC(C)(C)C]

To a solution of rac-tert-butyl (RS)-2-bromo-4-methyl-3-(pyridin-4-yl)-6,7-dihydropyrazolo[1,5-a]pyrazine-5(4H)-carboxylate (200 mg, 0.509 mmol, Intermediate 420) and 1H-indol-6-ylboronic acid (98.2 mg, 0.610 mmol) in 1,4-dioxane (10 ml) and water (2 ml) were added sodium carbonate (108 mg, 1.02 mmol) and 1,1′-bis(diphenylphosphino) ferrocene]dichloropalladium (II) (37.2 mg, 0.509 mmol) at 25° C. The mixture was stirred at 80° C. for 16 hours under nitrogen atmosphere. The reaction mixture was filtered and the filtrate was concentrated in vacuo to give a residue. The residue was purified by column chromatography on silica gel (300~400 mesh, petroleum ether:ethyl acetate=5:1 to 1:1) to rac-tert-butyl (RS)-2-(1H-indol-6-yl)-4-methyl-3-(pyridin-4-yl)-6,7-dihydropyrazolo[1,5-a]pyrazine-5(4H)-carboxylate (210 mg, 96% yield) as a yellow solid.

LC-MS (Method 14): Rₜ=0.831 minute; MS (ESIpos): m/z=430.2 [M+H]⁺.

### Intermediate 428

### rac-(RS)-2-(1H-indol-6-yl)-4-methyl-3-(pyridin-4-yl)-4,5,6,7-tetrahydropyrazolo[1,5-a]pyrazine hydrogen chloride (1/1)

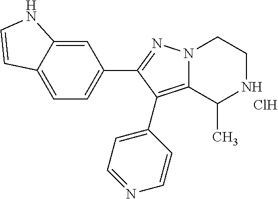 [SMILES: CC1NCCn2nc(-c3ccc4cc[nH]c4c3)c(-c3ccncc3)c21 ; Cl]

The reaction mixture of rac-tert-butyl (RS)-2-(1H-indol-6-yl)-4-methyl-3-(pyridin-4-yl)-6,7-dihydropyrazolo[1,5-a]pyrazine-5(4H)-carboxylate (190 mg, 0.442 mmol, Intermediate 427) in ethyl acetate (2 ml) was added hydrochloric acid (4M in ethyl acetate, 2 ml) at 25° C. After stirring at 25° C. for 1 hour, the reaction mixture was concentrated in vacuo to give rac-(RS)-2-(4-methoxyphenyl)-4-methyl-3-(pyridin-4-yl)-4,5,6,7-tetrahydropyrazolo[1,5-a]pyrazine hydrochloride (1:1) (160 mg, 99% yield) as a white solid.

LC-MS (Method 15): Rₜ=0.726 minute; MS (ESIpos): m/z=330.2 [M+H]⁺.

### Intermediate 429

### tert-butyl (6S)-2-(4-chlorophenyl)-6-methyl-6,7-dihydropyrazolo[1,5-a]pyrazine-5(4H)-carboxylate

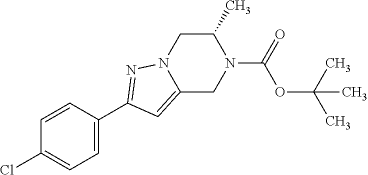 [SMILES: C[C@H]1Cn2nc(-c3ccc(Cl)cc3)cc2CN1C(=O)OC(C)(C)C]

To a solution of tert-butyl (6S)-2-bromo-6-methyl-6,7-dihydropyrazolo[1,5-a]pyrazine-5(4H)-carboxylate (3.00 g, 9.49 mmol, Intermediate 491) and (4-chlorophenyl) boronic acid (2.23 g, 14.2 mmol) in 1,4-dioxane (30 ml) and water (6 ml) were added [1,1-bis(diphenylphosphino) ferrocene]dichloropalladium (II) (694 mg, 0.949 mmol) and sodium carbonate (2.01 g, 19.0 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (6S)-2-(4-chlorophenyl)-6-methyl-6,7-dihydropyrazolo[1,5-a]pyrazine-5(4H)-carboxylate (3.00 g, 8.62 mmol, 91% yield) as a yellow oil.

LC-MS (Method 16): Rₜ=0.945 min; MS (ESIpos): m/z=348.1 [M+H]⁺

### Intermediate 430

### tert-butyl (6S)-2-(4-chlorophenyl)-3-iodo-6-methyl-6,7-dihydropyrazolo[1,5-a]pyrazine-5(4H)-carboxylate

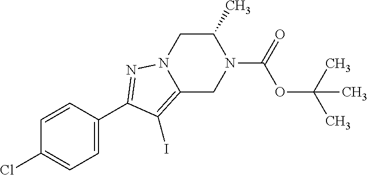 [SMILES: C[C@H]1Cn2nc(-c3ccc(Cl)cc3)c(I)c2CN1C(=O)OC(C)(C)C]

To a solution of tert-butyl (6S)-2-(4-chlorophenyl)-6-methyl-6,7-dihydropyrazolo[1,5-a]pyrazine-5(4H)-carboxylate (3.00 g, 8.62 mmol, Intermediate 429) in dichloromethane (36 ml) and methanol (18 ml) was added N-iodosuccinimide (3.88 g, 17.2 mmol) at 20° C. The mixture was stirred at 45° C. for 16 hours. The reaction solution was washed with saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:1) to give tert-butyl (6S)-2-(4-chlorophenyl)-3-iodo-6-methyl-6,7-dihydropyrazolo[1,5-a]pyrazine-5(4H)-carboxylate (4.00 g, 8.44 mmol, 98% yield) as a yellow oil.

LC-MS (Method 16): Rₜ=1.102 min; MS (ESIpos): m/z=474.0 [M+H]⁺

### Intermediate 431

### tert-butyl (6S)-2-(4-chlorophenyl)-6-methyl-3-(pyridin-4-yl)-6,7-dihydropyrazolo[1,5-a]pyrazine-5(4H)-carboxylate

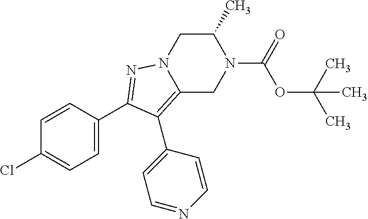 [SMILES: C[C@H]1Cn2nc(-c3ccc(Cl)cc3)c(-c3ccncc3)c2CN1C(=O)OC(C)(C)C]

To a solution of tert-butyl (6S)-2-(4-chlorophenyl)-3-iodo-6-methyl-6,7-dihydropyrazolo[1,5-a]pyrazine-5(4H)-carboxylate (4.00 g, 8.44 mmol, Intermediate 430) and pyridin-4-ylboronic acid (1.25 g. 10.1 mmol) in 1,4-dioxane (32 ml) and water (6.4 ml) were added [1,1-bis(diphenylphosphino) ferrocene]dichloropalladium (II) (618 mg, 0.844 mmol) and sodium carbonate (1.79 g, 16.9 mmol) at 20° C. The mixture was stirred at 80° C. for 16 hours under nitrogen atmosphere. The reaction solution was washed with water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (6S)-2-(4-chlorophenyl)-6-methyl-3-(pyridin-4-yl)-6,7-dihydropyrazolo[1,5-a]pyrazine-5(4H)-carboxylate (3.30 g, 7.77 mmol, 92% yield) as a yellow oil.

LC-MS (Method 16): Rₜ=0.482 min; MS (ESIpos): m/z=425.2 [M+H]⁺

### Intermediate 432

### (6S)-2-(4-chlorophenyl)-6-methyl-3-(pyridin-4-yl)-4,5,6,7-tetrahydropyrazolo[1,5-a]pyrazine hydrogen chloride (1/1)

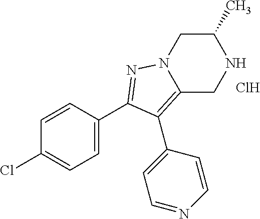 [SMILES: C[C@H]1Cn2nc(-c3ccc(Cl)cc3)c(-c3ccncc3)c2CN1 ; Cl]

A solution of tert-butyl (6S)-2-(4-chlorophenyl)-6-methyl-3-(pyridin-4-yl)-6,7-dihydropyrazolo[1,5-a]pyrazine-5(4H)-carboxylate (3.30 g, 7.77 mmol, Intermediate 431) in hydrochloric acid (4 M in ethyl acetate, 40 ml) was stirred at 20° C. for 1 hour. The reaction mixture was concentrated under reduced pressure to give (6S)-2-(4-chlorophenyl)-6-methyl-3-(pyridin-4-yl)-4,5,6,7-tetrahydropyrazolo[1,5-a]pyrazine hydrogen chloride (1/1) (2.80 g, 7.75 mmol, 100% yield) as a brown solid.

LC-MS (Method 16): Rₜ=0.409 min; MS (ESIpos): m/z=325.0 [(M−36)+H]⁺

### Intermediate 433

### tert-butyl (6R)-2-(4-chlorophenyl)-6-methyl-6,7-dihydropyrazolo[1,5-a]pyrazine-5(4H)-carboxylate

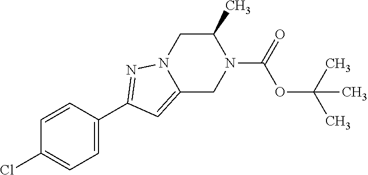 [SMILES: C[C@@H]1Cn2nc(-c3ccc(Cl)cc3)cc2CN1C(=O)OC(C)(C)C]

To a solution of tert-butyl (6R)-2-bromo-6-methyl-6,7-dihydropyrazolo[1,5-a]pyrazine-5(4H)-carboxylate (4.00 g, 12.7 mmol, Intermediate 496) and (4-chlorophenyl) boronic acid (2.97 g, 19.0 mmol) in 1,4-dioxane (30 ml) and water (6.0 ml) were added [1,1-bis(diphenylphosphino) ferrocene]dichloropalladium (II) (926 mg, 1.27 mmol) and sodium carbonate (2.68 g, 25.3 mmol) at 20° C. The mixture was stirred at 90° C. for 16 hours under nitrogen atmosphere. The reaction solution was washed with water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (6R)-2-(4-chlorophenyl)-6-methyl-6,7-dihydropyrazolo[1,5-a]pyrazine-5(4H)-carboxylate (4.00 g, 11.5 mmol, 91% yield) as a yellow oil.

LC-MS (Method 16): Rₜ=0.696 min; MS (ESIpos): m/z=348.2 [M+H]⁺

### Intermediate 434

### tert-butyl (6R)-2-(4-chlorophenyl)-3-iodo-6-methyl-6,7-dihydropyrazolo[1,5-a]pyrazine-5(4H)-carboxylate

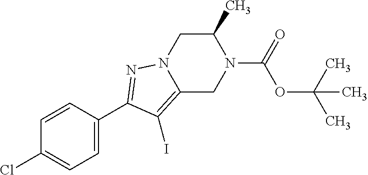 [SMILES: C[C@@H]1Cn2nc(-c3ccc(Cl)cc3)c(I)c2CN1C(=O)OC(C)(C)C]

To a solution of tert-butyl (6R)-2-(4-chlorophenyl)-6-methyl-6,7-dihydropyrazolo[1,5-a]pyrazine-5(4H)-carboxylate (4.00 g, 11.5 mmol, Intermediate 433) in dichloromethane (60 ml) and methanol (20 ml) was added 1-iodopyrrolidine-2,5-dione (5.17 g, 23.0 mmol) at 20° C. The mixture was stirred at 45° C. for 16 hours. The reaction solution was washed with saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:1) to give tert-butyl (6R)-2-(4-chlorophenyl)-3-iodo-6-methyl-6,7-dihydropyrazolo[1,5-a]pyrazine-5(4H)-carboxylate (5.20 g, 11.0 mmol, 95% yield) as a yellow oil.

LC-MS (Method 16): Rₜ=0.755 min; MS (ESIpos): m/z=474.1 [M+H]⁺

### Intermediate 435

### tert-butyl (6R)-2-(4-chlorophenyl)-6-methyl-3-(pyridin-4-yl)-6,7-dihydropyrazolo[1,5-a]pyrazine-5(4H)-carboxylate

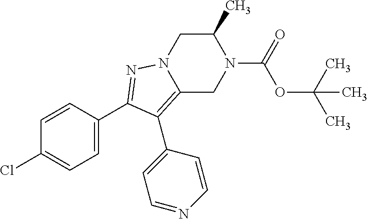 [SMILES: C[C@@H]1Cn2nc(-c3ccc(Cl)cc3)c(-c3ccncc3)c2CN1C(=O)OC(C)(C)C]

To a solution of tert-butyl (6R)-2-(4-chlorophenyl)-3-iodo-6-methyl-6,7-dihydropyrazolo[1,5-a]pyrazine-5(4H)-carboxylate (5.20 g, 11.0 mmol, Intermediate 434) and pyridin-4-ylboronic acid (2.70 g, 22.0 mmol) in 1,4-dioxane (48 ml) and water (9.6 ml) were added [1,1-bis(diphenylphosphino) ferrocene]dichloropalladium (II) (803 mg, 1.10 mmol) and sodium carbonate (2.33 g, 22.0 mmol) at 20° C. The mixture was stirred at 80° C. for 16 hours under nitrogen atmosphere. The reaction solution was washed with water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (6R)-2-(4-chlorophenyl)-6-methyl-3-(pyridin-4-yl)-6,7-dihydropyrazolo[1,5-a]pyrazine-5(4H)-carboxylate (4.20 g, 9.88 mmol, 90% yield) as a yellow oil.

LC-MS (Method 16): Rₜ=0.504 min; MS (ESIpos): m/z=425.2 [M+H]⁺.

### Intermediate 436

### (6R)-2-(4-chlorophenyl)-6-methyl-3-(pyridin-4-yl)-4,5,6,7-tetrahydropyrazolo[1,5-a]pyrazine hydrogen chloride (1/1)

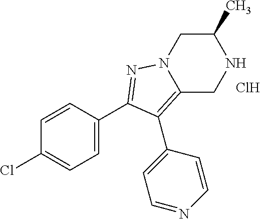 [SMILES: C[C@@H]1Cn2nc(-c3ccc(Cl)cc3)c(-c3ccncc3)c2CN1 ; Cl]

A solution of tert-butyl (6R)-2-(4-chlorophenyl)-6-methyl-3-(pyridin-4-yl)-6,7-dihydropyrazolo[1,5-a]pyrazine-5(4H)-carboxylate (4.20 g, 9.88 mmol, Intermediate 435) in hydrochloric acid (4 M in ethyl acetate, 40 ml) was stirred at 20° C. for 1 hour. The reaction mixture was concentrated under reduced pressure to give (6R)-2-(4-chlorophenyl)-6-methyl-3-(pyridin-4-yl)-4,5,6,7-tetrahydropyrazolo[1,5-a]pyrazine hydrogen chloride (1/1) (3.50 g, 9.69 mmol, 98% yield) as a brown solid.

LC-MS (Method 16): Rₜ=0.255 min; MS (ESIpos): m/z=325.1 [(M−36)+H]⁺.

### Intermediate 437

### methyl 3-bromo-1-{2-[(tert-butoxycarbonyl)amino]ethyl}-1H-pyrazole-5-carboxylate

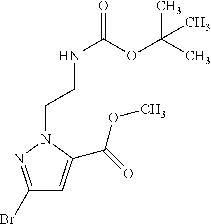 [SMILES: COC(=O)c1cc(Br)nn1CCNC(=O)OC(C)(C)C]

To a solution of methyl 3-bromo-1H-pyrazole-5-carboxylate (20.0 g, 97.6 mmol) in tetrahydrofuran (200 ml) were added triphenylphosphine (51.2 g, 127 mmol), tert-butyl(2-hydroxyethyl) carbamate (20.4 g, 127 mmol) and diisopropyl azodicarboxylate (39.5 g, 195 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and concentrated in vacuo to give a residue. The residue was purified by silica gel column chromatography (petroleum ether:ethyl acetate=1 to 3:1) to give methyl 3-bromo-1-{2-[(tert-butoxycarbonyl)amino]ethyl}-1H-pyrazole-5-carboxylate (34.0 g, 100% yield) as a white solid.

LC-MS (Method 16): Rₜ=0.912 min; MS (ESIpos): m/z=248.1 [(M−100)+H]⁺

### Intermediate 438

### 3-bromo-1-{2-[(tert-butoxycarbonyl)amino]ethyl}-1H-pyrazole-5-carboxylic acid

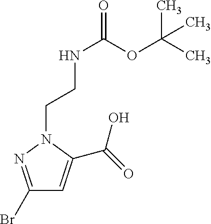 [SMILES: CC(C)(C)OC(=O)NCCn1nc(Br)cc1C(=O)O]

To a solution of methyl 3-bromo-1-{2-[(tert-butoxycarbonyl)amino]ethyl}-1H-pyrazole-5-carboxylate (38.0 g, 109 mmol, Intermediate 431) in ethanol (400 ml) was added a solution of potassium hydroxide (30.6 g, 546 mmol) in water (100 ml) at 20° C. The mixture was stirred at 20° C. for 16 hours. pH of the reaction mixture was adjusted with hydrochloric acid (1 M in water) to 6-7 and the resulting solution was extracted with ethyl acetate. The combined organic layers were washed with brine, dried over anhydrous sodium sulfate, filtered and concentrated in vacuo to give 3-bromo-1-{2-[(tert-butoxycarbonyl)amino]ethyl}-1H-pyrazole-5-carboxylic acid (33.5 g, 100 mmol, 92% yield) as a white solid.

LC-MS (Method 16): Rₜ=0.809 min; MS (ESIpos): m/z=236.0 [(M−100)+H]⁺.

### Intermediate 439

### tert-butyl(2-{3-bromo-5-[methoxy(methyl) carbamoyl]-1H-pyrazol-1-yl}ethyl) carbamate

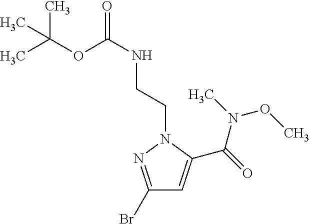 [SMILES: CON(C)C(=O)c1cc(Br)nn1CCNC(=O)OC(C)(C)C]

To a solution of 3-bromo-1-{2-[(tert-butoxycarbonyl)amino]ethyl}-1H-pyrazole-5-carboxylic acid (33.0 g, 98.8 mmol, Intermediate 438) and O-(7-azabenzotriazol-1-yl)-N,N,N,N-tetramethyl uronium hexafluorophosphate (56.3 g, 148 mmol) in N,N-dimethylformamide (500 ml) were added triethylamine (55 ml, 400 mmol) and N-methoxymethanamine hydrogen chloride (1/1) (19.3 g, 198 mmol) at 20° C. The mixture was stirred at 20° C. for 16 hours. The reaction solution was poured into sodium hydrogencarbonate aqueous solution and extracted with ethyl acetate. The combined organic layers were washed with brine, dried over anhydrous sodium sulfate, filtered and concentrated in vacuo to give tert-butyl(2-{3-bromo-5-[methoxy(methyl) carbamoyl]-1H-pyrazol-1-yl}ethyl) carbamate (26.0 g, 68.9 mmol, 70% yield) as a yellow oil.

LC-MS (Method 16): Rₜ=0.892 min; MS (ESIpos): m/z=279.1 [(M−100)+H]⁺

### Intermediate 440

### tert-butyl [2-(5-acetyl-3-bromo-1H-pyrazol-1-yl)ethyl]carbamate

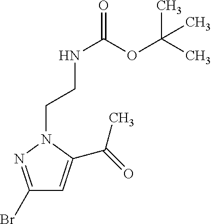 [SMILES: CC(=O)c1cc(Br)nn1CCNC(=O)OC(C)(C)C]

To a solution of tert-butyl(2-{3-bromo-5-[methoxy(methyl) carbamoyl]-1H-pyrazol-1-yl}ethyl) carbamate (26.0 g, 68.9 mmol, Intermediate 439) in tetrahydrofuran (380 ml) was added bromo(methyl) magnesium (3.0 M in tetrahydrofuran, 53 ml, 160 mmol) at −78° C. under nitrogen atmosphere. The mixture was stirred at 20° C. for 2 hours. The reaction was quenched with saturated ammonium chloride aqueous solution and extracted with ethyl acetate. The combined organic layers were washed with brine, dried over anhydrous sodium sulfate, filtered and concentrated in vacuo to give a residue. The residue was purified by silica gel column chromatography (petroleum ether:ethyl acetate=2:1 to 3:2) to give tert-butyl [2-(5-acetyl-3-bromo-1H-pyrazol-1-yl)ethyl]carbamate (20.0 g, 60.2 mmol, 87% yield) as a colorless oil.

LC-MS (Method 16): Rₜ=0.512 min; MS (ESIpos): m/z=232.0 [M+H]⁺.

### Intermediate 441

### 2-bromo-4-methyl-6,7-dihydropyrazolo[1,5-a]pyrazine

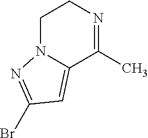

To a solution of tert-butyl [2-(5-acetyl-3-bromo-1H-pyrazol-1-yl)ethyl]carbamate (21.0 g, 63.2 mmol, Intermediate 440) in dichloromethane (150 ml) was added trifluoroacetic acid (50 ml, 649 mmol) at 20° C. The mixture was stirred at 20° C. for 16 hours. The reaction mixture was concentrated under reduced pressure to give 2-bromo-4-methyl-6,7-dihydropyrazolo[1,5-a]pyrazine (14.0 g, 65.4 mmol, 103% yield) as a brown oil.

LC-MS (Method 14): Rₜ=0.327 min; MS (ESIpos): m/z=214.0 [M+H]⁺

### Intermediate 442

### (4RS)-2-bromo-4-methyl-4,5,6,7-tetrahydropyrazolo[1,5-a]pyrazine

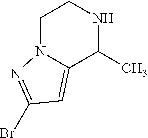

To a solution of 2-bromo-4-methyl-6,7-dihydropyrazolo[1,5-a]pyrazine (14.0 g, 65.4 mmol, Intermediate 441) in methanol (200 ml) was added sodium cyanoborohydride (20.5 g, 327 mmol) at 20° C. The mixture was stirred at 20° C. for 16 hours. The reaction mixture was poured into water and extracted with ethyl acetate. The combined organic layers were washed with brine, dried over anhydrous sodium sulfate, filtered and concentrated in vacuo to give racemic (4RS)-2-bromo-4-methyl-4,5,6,7-tetrahydropyrazolo[1,5-a]pyrazine (14.0 g, 64.8 mmol, 99% yield) as a yellow oil.

### Intermediate 443

### tert-butyl (4RS)-2-bromo-4-methyl-6,7-dihydropyrazolo[1,5-a]pyrazine-5(4H)-carboxylate

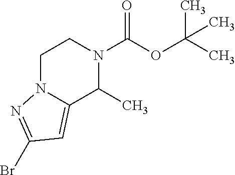 [SMILES: CC1c2cc(Br)nn2CCN1C(=O)OC(C)(C)C]

To a solution of (4RS)-2-bromo-4-methyl-4,5,6,7-tetrahydropyrazolo[1,5-a]pyrazine (14.0 g, 64.8 mmol, Intermediate 442) in dichloromethane (200 ml) were added triethylamine (27 ml, 194 mmol) and di-tert-butyl dicarbonate (30 ml, 130 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:2) to give tert-butyl (4RS)-2-bromo-4-methyl-6,7-dihydropyrazolo[1,5-a]pyrazine-5(4H)-carboxylate (10.0 g, 31.6 mmol, 49% yield) as a white oil.

LC-MS (Method 14): Rₜ=0.992 min; MS (ESIpos): m/z=318.3 [M+H]⁺.

### Intermediate 444

### tert-butyl (4RS)-4-methyl-2-[4-(trifluoromethyl)phenyl]-6,7-dihydropyrazolo[1,5-a]pyrazine-5(4H)-carboxylate

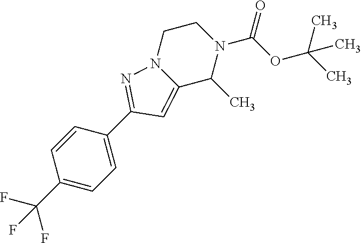 [SMILES: CC1c2cc(-c3ccc(C(F)(F)F)cc3)nn2CCN1C(=O)OC(C)(C)C]

To a solution of tert-butyl (4RS)-2-bromo-4-methyl-6,7-dihydropyrazolo[1,5-a]pyrazine-5(4H)-carboxylate (3.00 g, 9.49 mmol, Intermediate 443) and [4-(trifluoromethyl)phenyl]boronic acid (2.70 g, 14.2 mmol) in 1,4-dioxane (30 ml) and water (6.0 ml) were added [1,1-bis(diphenylphosphino) ferrocene]dichloropalladium (II) (694 mg, 0.949 mmol) and sodium carbonate (2.01 g, 19.0 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:2) to give tert-butyl (4RS)-4-methyl-2-[4-(trifluoromethyl)phenyl]-6,7-dihydropyrazolo[1,5-a]pyrazine-5(4H)-carboxylate (3.00 g, 7.87 mmol, 83% yield) as a yellow oil.

LC-MS (Method 16): Rₜ=0.689 min; MS (ESIpos): m/z=382.1 [M+H]⁺.

### Intermediate 445

### tert-butyl (4RS)-3-iodo-4-methyl-2-[4-(trifluoromethyl)phenyl]-6,7-dihydropyrazolo[1,5-a]pyrazine-5(4H)-carboxylate

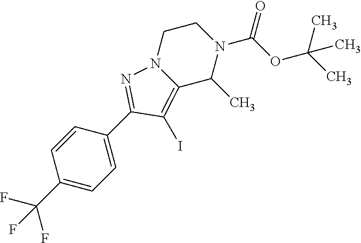 [SMILES: CC1c2c(I)c(-c3ccc(C(F)(F)F)cc3)nn2CCN1C(=O)OC(C)(C)C]

To a solution of tert-butyl (4RS)-4-methyl-2-[4-(trifluoromethyl)phenyl]-6,7-dihydropyrazolo[1,5-a]pyrazine-5(4H)-carboxylate (3.00 g, 7.87 mmol, Intermediate 444) in dichloromethane (30 ml) and methanol (15 ml) was added N-iodosuccinimide (2.65 g, 11.8 mmol) at 20° C. The mixture was stirred at 45° C. for 16 hour. The reaction solution was washed with saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=9:1 to 6:1) to give tert-butyl (4RS)-3-iodo-4-methyl-2-[4-(trifluoromethyl)phenyl]-6,7-dihydropyrazolo[1,5-a]pyrazine-5(4H)-carboxylate (4.20 g, 8.28 mmol, 105% yield) as a white oil.

LC-MS (Method 16): Rₜ=0.736 min; MS (ESIpos): m/z=508.1 [M+H]⁺

### Intermediate 446

### tert-butyl 3-iodo-4-methyl-2-[4-(trifluoromethyl)phenyl]-6,7-dihydropyrazolo[1,5-a]pyrazine-5(4H)-carboxylate (isomer 2)

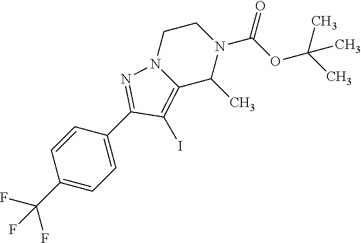 [SMILES: CC1c2c(I)c(-c3ccc(C(F)(F)F)cc3)nn2CCN1C(=O)OC(C)(C)C]

tert-butyl (4RS)-3-iodo-4-methyl-2-[4-(trifluoromethyl)phenyl]-6,7-dihydropyrazolo[1,5-a]pyrazine-5(4H)-carboxylate (4.00 g, 7.88 mmol, Intermediate 445) was separated by chiral separation. [Column: Chiralpak AD-3 50×4.6 mm I.D., 3 μm Mobile phase: Phase A for CO2, and Phase B for IPA (0.05% DEA); Gradient elution: IPA (0.05% DEA) in CO2 from 5% to 40% Flow rate: 3 mL/min; Detector: PDA; Column Temp: 35 C; Back Pressure: 100 Bar] to give tert-butyl 3-iodo-4-methyl-2-[4-(trifluoromethyl)phenyl]-6,7-dihydropyrazolo[1,5-a]pyrazine-5(4H)-carboxylate (isomer 2) (1.60 g, 3.09 mmol, 98% purity, 39% yield) as a white oil.

Optical rotation: [α]_{D}=+82.724° (c=0.724 g/100 ml,methanol)

### Intermediate 447

### tert-butyl 4-methyl-3-(1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 2)

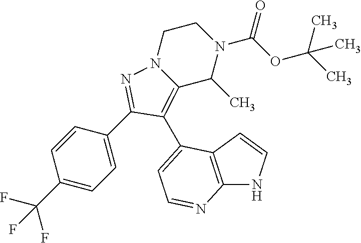 [SMILES: CC1c2c(-c3ccnc4[nH]ccc34)c(-c3ccc(C(F)(F)F)cc3)nn2CCN1C(=O)OC(C)(C)C]

To a solution of tert-butyl 3-iodo-4-methyl-2-[4-(trifluoromethyl)phenyl]-6,7-dihydropyrazolo[1,5-a]pyrazine-5(4H)-carboxylate (isomer 2) (400 mg, 0.788 mmol, Intermediate 446) and 1H-pyrrolo[2,3-b]pyridin-4-ylboronic acid (383 mg, 2.37 mmol) in 1,4-dioxane (5 mi) and water (1 ml) were added [1,1-bis(diphenylphosphino) ferrocene]dichloropalladium (II) (57.7 mg, 0.0788 mmol) and sodium carbonate (167 mg, 1.58 mmol) at 20° C. The mixture was stirred at 90° C. for 16 hours under nitrogen atmosphere. The reaction solution was washed with water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=3:2 to 1:1) to give tert-butyl 4-methyl-3-(1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 2) (260 mg, 0.523 mmol, 66% yield) as a yellow oil.

LC-MS (Method 16): Rₜ=0.592 min; MS (ESIpos): m/z=498.2 [M+H]⁺

### Intermediate 448

### 4-methyl-3-(1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2)

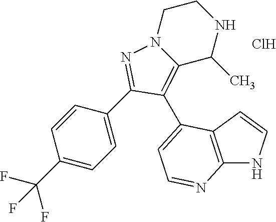 [SMILES: CC1NCCn2nc(-c3ccc(C(F)(F)F)cc3)c(-c3ccnc4[nH]ccc34)c21 ; Cl]

A solution of tert-butyl 4-methyl-3-(1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 2) (260 mg, 0.523 mmol, Intermediate 447) in hydrochloric acid (4 M in ethyl acetate, 4.0 ml) was stirred at 20° C. for 1 hours. The reaction mixture was concentrated under reduced pressure to give 4-methyl-3-(1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2) (200 mg, 0.503 mmol, 96% yield) as a brown solid.

LC-MS (Method 16): Rₜ=0.744 min; MS (ESIpos): m/z=398.1 [M+H]⁺

### Intermediate 449

### tert-butyl (6S)-6-methyl-2-(4-(trifluoromethyl)phenyl)-6,7-dihydropyrazolo[1,5-a]pyrazine-5(4H)-carboxylate

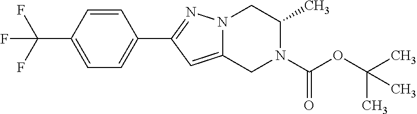 [SMILES: C[C@H]1Cn2nc(-c3ccc(C(F)(F)F)cc3)cc2CN1C(=O)OC(C)(C)C]

To a solution of tert-butyl (6S)-2-bromo-6-methyl-6,7-dihydropyrazolo[1,5-a]pyrazine-5(4H)-carboxylate (400 mg, 1.27 mmol, Intermediate 491) and [4-(trifluoromethyl)phenyl]boronic acid (360 mg, 1.90 mmol) in 1,4-dioxane (4.0 ml) and water (0.8 ml) were added sodium carbonate (268 mg, 2.53 mmol) and (1,1′-bis(diphenylphosphino) ferrocene) palladium (II) dichloride (92.6 mg, 127 μmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=3:2 to 1:1) to give tert-butyl (6S)-6-methyl-2-[4-(trifluoromethyl)phenyl]-6,7-dihydropyrazolo[1,5-a]pyrazine-5(4H)-carboxylate (500 mg, 1.31 mmol, 104% yield) as a yellow oil.

LC-MS (Method 16): Rₜ=0.691 min; MS (ESIpos): m/z=382.1 [M+H]⁺

### Intermediate 450

### tert-butyl (6S)-3-iodo-6-methyl-2-[4-(trifluoromethyl)phenyl]-6,7-dihydropyrazolo[1,5-a]pyrazine-5(4H)-carboxylate

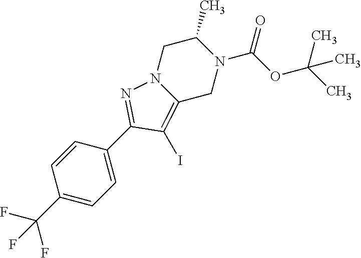 [SMILES: C[C@H]1Cn2nc(-c3ccc(C(F)(F)F)cc3)c(I)c2CN1C(=O)OC(C)(C)C]

To a solution of tert-butyl (6S)-6-methyl-2-[4-(trifluoromethyl)phenyl]-6,7-dihydropyrazolo[1,5-a]pyrazine-5(4H)-carboxylate (500 mg, 1.31 mmol, Intermediate 449) in dichloromethane (5.0 ml) and methanol (2.5 ml) was added N-iodosuccinimide (590 mg, 2.62 mmol) at 20° C. The mixture was stirred at 45° C. for 16 hours. The reaction solution was washed with saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=9:1 to 6; 1) to give tert-butyl (6S)-3-iodo-6-methyl-2-[4-(trifluoromethyl)phenyl]-6,7-dihydropyrazolo[1,5-a]pyrazine-5(4H)-carboxylate (550 mg, 1.08 mmol, 83% yield) as a white oil.

LC-MS (Method 16): Rₜ=0.677 min; MS (ESIpos): m/z=508.2 [M+H]⁺

### Intermediate 451

### tert-butyl (6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate

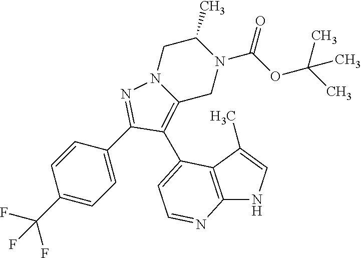 [SMILES: Cc1c[nH]c2nccc(-c3c(-c4ccc(C(F)(F)F)cc4)nn4c3CN(C(=O)OC(C)(C)C)[C@@H](C)C4)c12]

To a solution of tert-butyl (6S)-3-iodo-6-methyl-2-[4-(trifluoromethyl)phenyl]-6,7-dihydropyrazolo[1,5-a]pyrazine-5(4H)-carboxylate (400 mg, 0.788 mmol, Intermediate 450) and (3-methyl-1H-pyrrolo[2,3-b]pyridin-4-yl) boronic acid (416 mg, 2.37 mmol, Intermediate 245) in 1,4-dioxane (5.0 ml) and water (1.0 ml) were added [1,1-bis(diphenylphosphino) ferrocene]dichloropalladium (II) (57.7 mg, 0.0788 mmol) and sodium carbonate (167 mg, 1.58 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (200 mg, 0.391 mmol, 50% yield) as a yellow oil.

LC-MS (Method 16): Rₜ=0.594 min; MS (ESIpos): m/z=512.3 [M+H]⁺.

### Intermediate 452

### (6S)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1)

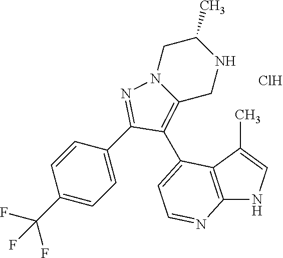 [SMILES: Cc1c[nH]c2nccc(-c3c(-c4ccc(C(F)(F)F)cc4)nn4c3CN[C@@H](C)C4)c12 ; Cl]

A solution of tert-butyl (6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (200 mg, 0.391 mmol, Intermediate 451) in hydrochloric acid (4 M in ethyl acetate, 3.0 ml) was stirred at 20° C. for 1 hour. The reaction mixture was concentrated under reduced pressure to give (6S)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (200 mg, 0.447 mmol, 114% yield) as a brown solid.

LC-MS (Method 16): Rₜ=0.759 min; MS (ESIpos): m/z=412.2 [(M−36)+H]⁺.

### Intermediate 453

### tert-butyl (4RS)-2-(4-fluorophenyl)-4-methyl-6,7-dihydropyrazolo[1,5-a]pyrazine-5(4H)-carboxylate

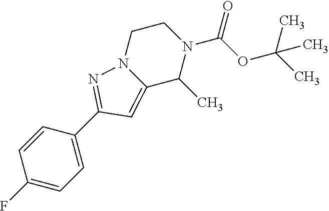 [SMILES: CC1c2cc(-c3ccc(F)cc3)nn2CCN1C(=O)OC(C)(C)C]

To a solution of (4RS)-2-bromo-4-methyl-6,7-dihydropyrazolo[1,5-a]pyrazine-5(4H)-carboxylate (3.00 g, 9.49 mmol, Intermediate 443) and (4-fluorophenyl) boronic acid (1.99 g, 14.2 mmol) in 1,4-dioxane (30 ml) and water (6 ml) were added (1,1′-bis(diphenylphosphino) ferrocene) palladium (II) dichloride (694 mg, 0.949 mmol) and sodium carbonate (2.01 g, 19.0 mmol) at 20° C. The mixture was stirred at 90° C. for 16 hours under nitrogen atmosphere. The reaction solution was washed with water and extracted with ethyl acetate. The combined organic layers were washed with brine, dried over anhydrous sodium sulfate, filtered and concentrated in vacuo to give a residue. The residue was purified by silica gel column chromatography (petroleum ether:ethyl acetate=4:1 to 3:2) to give tert-butyl (4RS)-2-(4-fluorophenyl)-4-methyl-6,7-dihydropyrazolo[1,5-a]pyrazine-5(4H)-carboxylate (2.60 g, 7.85 mmol, 83% yield) as a yellow oil.

LC-MS (Method 16): Rₜ=0.649 min; MS (ESIpos): m/z=332.1 [M+H]⁺.

### Intermediate 454

### tert-butyl (4RS)-2-(4-fluorophenyl)-3-iodo-4-methyl-6,7-dihydropyrazolo[1,5-a]pyrazine-5(4H)-carboxylate

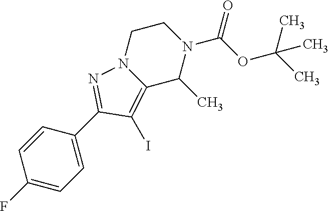 [SMILES: CC1c2c(I)c(-c3ccc(F)cc3)nn2CCN1C(=O)OC(C)(C)C]

To a solution of racemic (4RS)-2-(4-fluorophenyl)-4-methyl-6,7-dihydropyrazolo[1,5-a]pyrazine-5(4H)-carboxylate (2.60 g, 7.85 mmol, Intermediate 453) in dichloromethane (30 ml) and methanol (15 ml) was added N-iodosuccinimide (2.65 g, 11.8 mmol) at 20° C. The mixture was stirred at 45° C. for 16 hour. The reaction solution was washed with saturated sodium sulfite aqueous solution and extracted with ethyl acetate. The combined organic layers were washed with brine, dried over anhydrous sodium sulfate, filtered and concentrated in vacuo to give a residue. The residue was purified by silica gel column chromatography (petroleum ether:ethyl acetate=9:1 to 6:1) to give racemic tert-butyl (4RS)-2-(4-fluorophenyl)-3-iodo-4-methyl-6,7-dihydropyrazolo[1,5-a]pyrazine-5(4H)-carboxylate (3.80 g, 8.31 mmol, 106% yield) as a white oil.

LC-MS (Method 16): Rₜ=0.701 min; MS (ESIpos): m/z=458.1 [M+H]⁺.

### Intermediate 455

### tert-butyl 2-(4-fluorophenyl)-3-iodo-4-methyl-6,7-dihydropyrazolo[1,5-a]pyrazine-5(4H)-carboxylate (isomer 1)

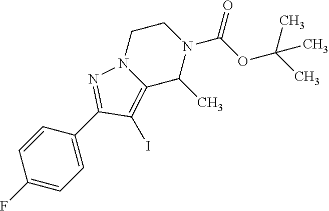 [SMILES: CC1c2c(I)c(-c3ccc(F)cc3)nn2CCN1C(=O)OC(C)(C)C]

tert-butyl (4RS)-2-(4-fluorophenyl)-3-iodo-4-methyl-6,7-dihydropyrazolo[1,5-a]pyrazine-5(4H)-carboxylate (3.80 g, 8.31 mmol, Intermediate 454) was separated by chiral separation [Column: Chiralcel OJ-3 50×4.6 mm I.D., 3 μm; Mobile phase: Phase A for CO2, and Phase B for IPA (0.05% DEA); Gradient elution: IPA (0.05% DEA) in CO2 from 5% to 40% Flow rate: 3 mL/min; Detector: PDA; Column Temp: 35 C; Back Pressure: 100 Bar] to give tert-butyl 2-(4-fluorophenyl)-3-iodo-4-methyl-6,7-dihydropyrazolo[1,5-a]pyrazine-5(4H)-carboxylate (isomer 1) (1.80 g, 3.88 mmol, 47% yield) as a yellow oil. Optical rotation: [α]_{D}=−95.133° (c=0.39 g/100 ml, methanol)

LC-MS (Method 16): Rₜ=0.701 min; MS (ESIpos): m/z=458.1 [M+H]⁺.

### Intermediate 456

### tert-butyl 2-(4-fluorophenyl)-4-methyl-3-(1H-pyrrolo[2,3-b]pyridin-4-yl)-6,7-dihydropyrazolo[1,5-a]pyrazine-5(4H)-carboxylate (isomer 1)

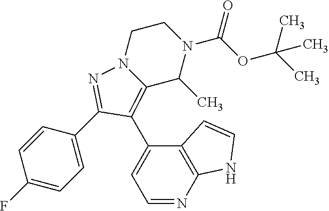 [SMILES: CC1c2c(-c3ccnc4[nH]ccc34)c(-c3ccc(F)cc3)nn2CCN1C(=O)OC(C)(C)C]

To a solution of tert-butyl 2-(4-fluorophenyl)-3-iodo-4-methyl-6,7-dihydropyrazolo[1,5-a]pyrazine-5(4H)-carboxylate (isomer 1) (400 mg, 0.875 mmol, Intermediate 455) and 1H-pyrrolo[2,3-b]pyridin-4-ylboronic acid (213 mg, 1.31 mmol) in 1,4-dioxane (8.0 ml) and water (2.0 ml) were added (1,1′-bis(diphenylphosphino) ferrocene) palladium (II) dichloride (64.0 mg, 0.0875 mmol) and sodium carbonate (185 mg, 1.75 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and the filtrate was concentrated in vacuo to give a residue. The residue was purified by silica gel column chromatography (petroleum ether:ethyl aceate=1:0 to 1:1) to give tert-butyl 2-(4-fluorophenyl)-4-methyl-3-(1H-pyrrolo[2,3-b]pyridin-4-yl)-6,7-dihydropyrazolo[1,5-a]pyrazine-5(4H)-carboxylate (isomer 1) (250 mg, 0.559 mmol, 64% yield) as a yellow oil.

LC-MS (Method 16): Rₜ=0.877 min; MS (ESIpos): m/z=448.6 [M+H]⁺.

### Intermediate 457

### 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 1)

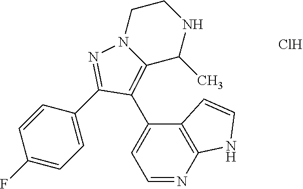 [SMILES: CC1NCCn2nc(-c3ccc(F)cc3)c(-c3ccnc4[nH]ccc34)c21 ; Cl]

To a solution of tert-butyl 2-(4-fluorophenyl)-4-methyl-3-(1H-pyrrolo[2,3-b]pyridin-4-yl)-6,7-dihydropyrazolo[1,5-a]pyrazine-5(4H)-carboxylate (isomer 1) (250 mg, 0.559 mmol, Intermediate 456) in ethyl acetate (8.0 ml) was added hydrochloric acid (4M in ethyl acetate, 8.0 ml) at 20° C. The reaction mixture was stirred at 20° C. for 2 hours. The reaction mixture was filtered and the solid cake was collected under vacuo to give 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 1) (200 mg, 0.521 mmol, 93% yield) as a brown solid.

LC-MS (Method 16): Rₜ=0.425 min; MS (ESIpos): m/z=348.1 [(M−36)+H]⁺.

### Intermediate 458

### tert-butyl 2-(4-fluorophenyl)-4-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (isomer 2)

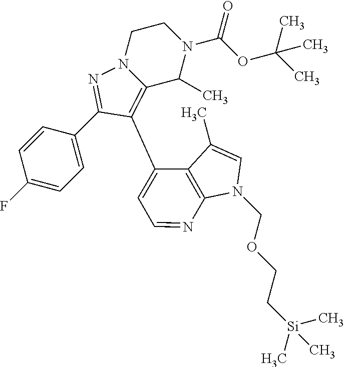 [SMILES: Cc1cn(COCC[Si](C)(C)C)c2nccc(-c3c(-c4ccc(F)cc4)nn4c3C(C)N(C(=O)OC(C)(C)C)CC4)c12]

To a solution of tert-butyl 2-(4-fluorophenyl)-3-iodo-4-methyl-6,7-dihydropyrazolo[1,5-a]pyrazine-5(4H)-carboxylate (isomer 2) (1.20 g, 2.62 mmol, Intermediate 455) and 3-methyl-4-(4,4,5,5-tetramethyl-1,3,2-dioxaborolan-2-yl)-1-{[2-(trimethylsilyl) ethoxy]methyl}-1H-pyrrolo[2,3-b]pyridine (1.12 g, 2.89 mmol, Intermediate 254) in 1,4-dioxane (24 ml) and water (4.8 ml) were added sodium carbonate (556 mg, 5.25 mmol) and [1,1-Bis(diphenylphosphino) ferrocene]dichloropalladium (II) (192 mg, 0.262 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 3:2) to give tert-butyl (4S)-2-(4-fluorophenyl)-4-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (isomer 2) (400 mg, 0.676 mmol, 26% yield) as a yellow oil.

LC-MS (Method 16): Rₜ=1.205 min; MS (ESIpos): m/z=592.3 [M+H]⁺

### Intermediate 459

### 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 2)

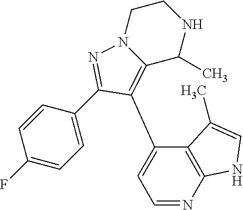 [SMILES: Cc1c[nH]c2nccc(-c3c(-c4ccc(F)cc4)nn4c3C(C)NCC4)c12]

A solution of tert-butyl 2-(4-fluorophenyl)-4-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (isomer 2) (400 mg, 0.676 mmol, Intermediate 458) in dichloromethane (3.0 ml) was added trifluoroacetic acid (3.0 ml) at 20° C. The mixture was stirred at 20° C. for 16 hours. The reaction mixture was concentrated under reduced pressure to give a residue. To this residue was added methanol (5 ml), followed by the addition of a solution of potassium carbonate (93.3 mg, 0.676 mmol) in water (5 ml). The resulting solution was stirred at 20° C. for 3 hours. The reaction solution was poured into water and extracted with ethyl acetate. The combined organic layers were washed with brine, dried over anhydrous sodium sulfate, filtered and concentrated in vacuo to give 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 2) (200 mg, 0.553 mmol, 82% yield) as a yellow oil.

LC-MS (Method 16): Rₜ=0.712 min; MS (ESIpos): m/z=362.2 [M+H]⁺

### Intermediate 460

### tert-butyl (6S)-2-(4-fluorophenyl)-6-methyl-6,7-dihydropyrazolo[1,5-a]pyrazine-5(4H)-carboxylate

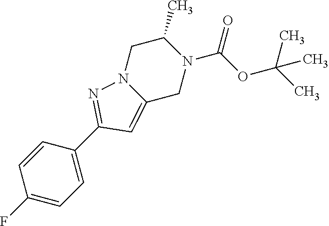 [SMILES: C[C@H]1Cn2nc(-c3ccc(F)cc3)cc2CN1C(=O)OC(C)(C)C]

To a solution of tert-butyl (6S)-2-bromo-6-methyl-6,7-dihydropyrazolo[1,5-a]pyrazine-5(4H)-carboxylate (300 mg, 0.949 mmol, Intermediate 491) and (4-fluorophenyl) boronic acid (199 mg, 1.42 mmol) in 1,4-dioxane (5.0 ml) and water (1.0 ml) were added [1,1-bis(diphenylphosphino) ferrocene]dichloropalladium (II) (69.4 mg, 0.0949 mmol) and sodium carbonate (201 mg, 1.90 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (6S)-2-(4-fluorophenyl)-6-methyl-6,7-dihydropyrazolo[1,5-a]pyrazine-5(4H)-carboxylate (310 mg, 0.935 mmol, 99% yield) as a yellow oil.

### Intermediate 461

### tert-butyl (6S)-2-(4-fluorophenyl)-3-iodo-6-methyl-6,7-dihydropyrazolo[1,5-a]pyrazine-5(4H)-carboxylate

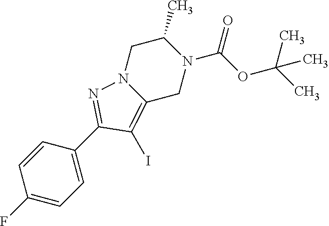 [SMILES: C[C@H]1Cn2nc(-c3ccc(F)cc3)c(I)c2CN1C(=O)OC(C)(C)C]

A solution of tert-butyl (6S)-2-(4-fluorophenyl)-6-methyl-6,7-dihydropyrazolo[1,5-a]pyrazine-5(4H)-carboxylate (300 mg, 0.905 μmol, Intermediate 460) and 1-iodopyrrolidine-2,5-dione (407 mg, 1.81 mmol) in dichloromethane (4.0 ml) and methanol (2.0 ml) were stirred at 20° C. for 16 hours. The reaction mixture was washed with saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:1) to give tert-butyl (6S)-2-(4-fluorophenyl)-3-iodo-6-methyl-6,7-dihydropyrazolo[1,5-a]pyrazine-5(4H)-carboxylate (320 mg, 0.700 mmol, 77% yield) as a yellow oil.

LC-MS (Method 16): Rₜ=1.048 min; MS (ESIpos): m/z=458.0 [M+H]⁺

### Intermediate 462

### tert-butyl (6S)-2-(4-fluorophenyl)-6-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

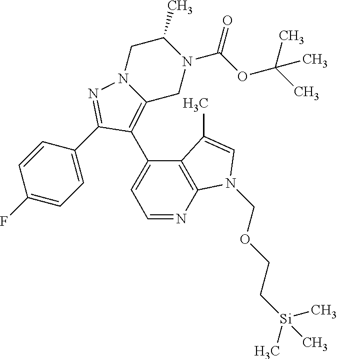 [SMILES: Cc1cn(COCC[Si](C)(C)C)c2nccc(-c3c(-c4ccc(F)cc4)nn4c3CN(C(=O)OC(C)(C)C)[C@@H](C)C4)c12]

To a solution of tert-butyl (6S)-2-(4-fluorophenyl)-3-iodo-6-methyl-6,7-dihydropyrazolo[1,5-a]pyrazine-5(4H)-carboxylate (320 mg, 0.700 mmol, Intermediate 461) and 3-methyl-4-(4,4,5,5-tetramethyl-1,3-dioxolan-2-yl)-1-{[2-(trimethylsilyl) ethoxy]methyl}-1H-pyrrolo[2,3-b]pyridine (547 mg, 1.40 mmol, Intermediate 254) in 1,4-dioxane (3.0 ml) and water (0.5 ml) were added [1,1-bis(diphenylphosphino) ferrocene]dichloropalladium (II) (51.2 mg, 0.0700 mmol) and sodium carbonate (148 mg, 1.40 mmol) at 20° C. The mixture was stirred at 80° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 3:2) to give tert-butyl (6S)-2-(4-fluorophenyl)-6-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (120 mg, 0.203 mmol, 29% yield) as a yellow oil.

LC-MS (Method 16): Rₜ=0.745 min; MS (ESIpos): m/z=592.3 [M+H]⁺

### Intermediate 463

### (6S)-2-(4-fluorophenyl)-6-methyl-3-(3-methyl-1H-pyrrolo[2,3-b]pyridin-4-yl)-4,5,6,7-tetrahydropyrazolo[1,5-a]pyrazine

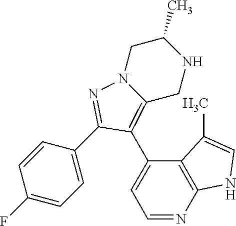 [SMILES: Cc1c[nH]c2nccc(-c3c(-c4ccc(F)cc4)nn4c3CN[C@@H](C)C4)c12]

A solution of tert-butyl (6S)-2-(4-fluorophenyl)-6-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (120 mg, 0.203 mmol, Intermediate 462) in trifluoroacetic acid (1.0 ml) and dichloromethane (3.0 ml) was stirred at 25° C. for 16 hours. The reaction mixture was concentrated under reduced pressure to give a residue. The residue was dissolved in methanol (5.0 ml), followed by the addition of potassium carbonate (56.0 mg, 0.406 mmol) in water (5.0 ml) at 25° C. The resulting mixture was stirred at 25° C. for 16 hours. The reaction mixture was poured into water and extracted with ethyl acetate. The combined organic layers were washed with brine, dried over anhydrous sodium sulfate, filtered and concentrated in vacuo to give (6S)-2-(4-fluorophenyl)-6-methyl-3-(3-methyl-1H-pyrrolo[2,3-b]pyridin-4-yl)-4,5,6,7-tetrahydropyrazolo[1,5-a]pyrazine (80 mg, 0.221 mmol, 109% yield) as a pink oil.

LC-MS (Method 16): Rₜ=0.677 min; MS (ESIpos): m/z=362.2 [M+H]⁺

### Intermediate 464

### tert-butyl 4-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 2)

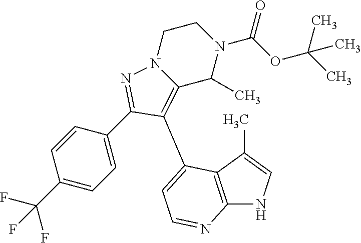 [SMILES: Cc1c[nH]c2nccc(-c3c(-c4ccc(C(F)(F)F)cc4)nn4c3C(C)N(C(=O)OC(C)(C)C)CC4)c12]

To a solution of tert-butyl 3-iodo-4-methyl-2-[4-(trifluoromethyl)phenyl]-6,7-dihydropyrazolo[1,5-a]pyrazine-5(4H)-carboxylate (isomer 2) (750 mg, 1.48 mmol, Intermediate 446) and (3-methyl-1H-pyrrolo[2,3-b]pyridin-4-yl) boronic acid (1.56 g, 8.87 mmol, Intermediate 245) in 1,4-dioxane (15 ml) and water (3 ml) were added sodium carbonate (313 mg 2.96 mmol) and [1,1-bis(diphenylphosphino) ferrocene]dichloropalladium (II) (108 mg, 0.148 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:2) to give tert-butyl 4-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 2) (100 mg, 0.195 mmol, 13% yield) as a yellow solid.

LC-MS (Method 16): Rₜ=0.827 min; MS (ESIpos): m/z=512.3 [M+H]⁺

### Intermediate 465

### 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2)

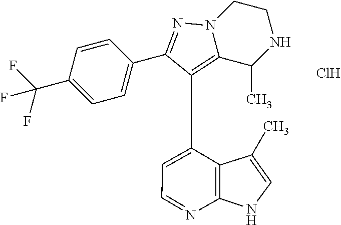 [SMILES: Cc1c[nH]c2nccc(-c3c(-c4ccc(C(F)(F)F)cc4)nn4c3C(C)NCC4)c12 ; Cl]

A solution of tert-butyl 4-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 2) (100 mg, 0.195 mmol, Intermediate 464) in hydrochloric acid (4 M in ethyl acetate, 4.0 ml) was stirred at 20° C. for 1 hour. The reaction mixture was concentrated under reduced pressure to give 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2) (80 mg, 0.179 mmol, 91% yield) as a brown solid.

LC-MS (Method 16): Rₜ=0.654 min; MS (ESIpos): m/z=412.3 [(M−36)+H]⁺

### Intermediate 466

### tert-butyl (6S)-2-bromo-3-iodo-6-methyl-6,7-dihydropyrazolo[1,5-a]pyrazine-5(4H)-carboxylate

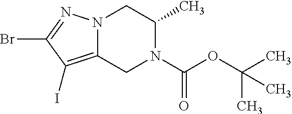 [SMILES: C[C@H]1Cn2nc(Br)c(I)c2CN1C(=O)OC(C)(C)C]

To a solution of tert-butyl (6S)-2-bromo-6-methyl-6,7-dihydropyrazolo[1,5-a]pyrazine-5(4H)-carboxylate (1.40 g, 4.43 mmol, Intermediate 491) in dichloromethane (10 ml) and methanol (5 ml) was added 1-iodopyrrolidine-2,5-dione (1.99 g, 8.86 mmol) at 20° C. The mixture was stirred at 45° C. for 16 hours. The reaction mixture was poured into saturated sodium sulfite aqueous solution and extracted with ethyl acetate. The combined organic phase was washed with brine, dried over anhydrous sodium sulfate, filtered and concentrated in vacuo to give a residue. The residue was purified by column chromatography on silica gel (petroleum ether:ethyl acetate=2:1 to 3:2) to give tert-butyl (6S)-2-bromo-3-iodo-6-methyl-6,7-dihydropyrazolo[1,5-a]pyrazine-5(4H)-carboxylate (1.90 g, 4.30 mmol, 97% yield) as a white solid.

LC-MS (Method 16): Rₜ=0.917 min; MS (ESIpos): m/z=441.9 [M+H]⁺.

### Intermediate 467

### tert-butyl (6S)-2-bromo-6-methyl-3-(3-methyl-1H-pyrrolo[2,3-b]pyridin-4-yl)-6,7-dihydropyrazolo[1,5-a]pyrazine-5(4H)-carboxylate

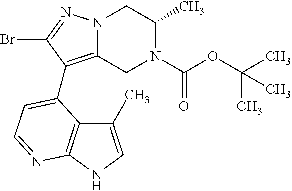 [SMILES: Cc1c[nH]c2nccc(-c3c(Br)nn4c3CN(C(=O)OC(C)(C)C)[C@@H](C)C4)c12]

To a solution of tert-butyl (6S)-2-bromo-3-iodo-6-methyl-6,7-dihydropyrazolo[1,5-a]pyrazine-5(4H)-carboxylate (500 mg, 1.13 mmol, Intermediate 466) and (3-methyl-1H-pyrrolo[2,3-b]pyridin-4-yl) boronic acid (498 mg, 2.83 mmol, Intermediate 245) in 1,4-dioxane (5.0 ml) and water (1.0 ml) were added [1,1-bis(diphenylphosphino) ferrocene]dichloropalladium (II) (82.8 mg, 0.113 mmol) and sodium carbonate (240 mg, 2.26 mmol) at 20° C. The mixture was stirred at 80° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=3:2 to 1:1) to give tert-butyl (6S)-2-bromo-6-methyl-3-(3-methyl-1H-pyrrolo[2,3-b]pyridin-4-yl)-6,7-dihydropyrazolo[1,5-a]pyrazine-5(4H)-carboxylate (320 mg, 0.717 mmol, 63% yield) as a yellow oil.

LC-MS (Method 16): Rₜ=0.532 min; MS (ESIpos): m/z=448.1 [M+H]⁺.

### Intermediate 468

### tert-butyl (6S)-2-(4-fluorophenyl)-6-methyl-3-(3-methyl-1H-pyrrolo[2,3-b]pyridin-4-yl)-6,7-dihydropyrazolo[1,5-a]pyrazine-5(4H)-carboxylate

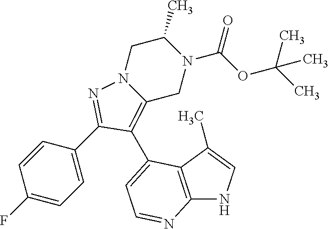 [SMILES: Cc1c[nH]c2nccc(-c3c(-c4ccc(F)cc4)nn4c3CN(C(=O)OC(C)(C)C)[C@@H](C)C4)c12]

To a solution of tert-butyl (6S)-2-bromo-6-methyl-3-(3-methyl-1H-pyrrolo[2,3-b]pyridin-4-yl)-6,7-dihydropyrazolo[1,5-a]pyrazine-5(4H)-carboxylate (150 mg, 0.336 mmol, Intermediate 467) and (4-fluorophenyl) boronic acid (235 mg, 1.68 mmol) in 1,4-dioxane (15 ml) and water (3 ml) were added [1,1-bis(diphenylphosphino) ferrocene]dichloropalladium (II) (24.6 mg, 0.0336 mmol) and sodium carbonate (71.2 mg, 0.672 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by flash reversed-phase MPLC (MeCN/H2O, 0.5% formic acid, 45%~55%) to give tert-butyl (6S)-2-(4-fluorophenyl)-6-methyl-3-(3-methyl-1H-pyrrolo[2,3-b]pyridin-4-yl)-6,7-dihydropyrazolo[1,5-a]pyrazine-5(4H)-carboxylate (40.0 mg, 0.0867 mmol, 26% yield) as a brown solid.

LC-MS (Method 16): Rₜ=0.507 min; MS (ESIpos): m/z=462.3 [M+H]⁺

### Intermediate 469

### (6S)-2-(4-fluorophenyl)-6-methyl-3-(3-methyl-1H-pyrrolo[2,3-b]pyridin-4-yl)-4,5,6,7-tetrahydropyrazolo[1,5-a]pyrazine hydrochloride (1:1)

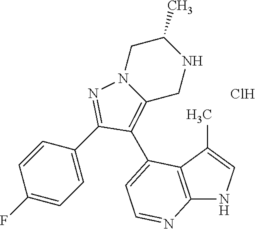 [SMILES: Cc1c[nH]c2nccc(-c3c(-c4ccc(F)cc4)nn4c3CN[C@@H](C)C4)c12 ; Cl]

A solution of tert-butyl (6S)-2-(4-fluorophenyl)-6-methyl-3-(3-methyl-1H-pyrrolo[2,3-b]pyridin-4-yl)-6,7-dihydropyrazolo[1,5-a]pyrazine-5(4H)-carboxylate (40.0 mg, 0.0867 mmol, Intermediate 468) in hydrochloric acid (4 M in ethyl acetate, 3.0 ml) was stirred at 20° C. for 1 hour. The reaction mixture was concentrated under reduced pressure to give (6S)-2-(4-fluorophenyl)-6-methyl-3-(3-methyl-1H-pyrrolo[2,3-b]pyridin-4-yl)-4,5,6,7-tetrahydropyrazolo[1,5-a]pyrazine hydrogen chloride (1/1) (40 mg, 0.101 mmol, 116% yield) as a brown solid.

LC-MS (Method 16): Rₜ=0.423 min; MS (ESIpos): m/z=362.1 [(M−36)+H]⁺.

### Intermediate 470

### tert-butyl-4-methyl-3-(1H-pyrrolo[2,3-b]pyridin-4-yl)-2-(4-(trifluoromethyl)phenyl)-6,7-dihydropyrazolo[1,5-a]pyrazine-5(4H)-carboxylate (isomer 2)

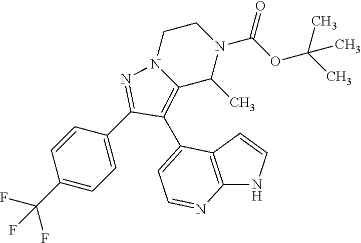 [SMILES: CC1c2c(-c3ccnc4[nH]ccc34)c(-c3ccc(C(F)(F)F)cc3)nn2CCN1C(=O)OC(C)(C)C]

To a solution of tert-butyl-3-iodo-4-methyl-2-[4-(trifluoromethyl)phenyl]-6,7-dihydropyrazolo[1,5-a]pyrazine-5(4H)-carboxylate (isomer 2) (800 mg, 1.58 mmol, Intermediate 446) and 1H-pyrrolo[2,3-b]pyridin-4-ylboronic acid (511 mg, 3.15 mmol) in 1,4-dioxane (10 ml) and water (2 ml) were added sodium carbonate (334 mg, 3.15 mmol) and [1,1-bis(diphenylphosphino) ferrocene]dichloropalladium (II) (115 mg, 0.158 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 1:1) to give tert-butyl-4-methyl-3-(1H-pyrrolo[2,3-b]pyridin-4-yl)-2-(4-(trifluoromethyl)phenyl)-6,7-dihydropyrazolo[1,5-a]pyrazine-5(4H)-carboxylate (isomer 2) (500 mg, 1.00 mmol, 64% yield) as a yellow oil.

LC-MS (Method 16): Rₜ=0.842 min; MS (ESIpos): m/z=498.2 [M+H]⁺

### Intermediate 471

### 4-methyl-3-(1H-pyrrolo[2,3-b]pyridin-4-yl)-2-(4-(trifluoromethyl)phenyl)-4,5,6,7-tetrahydropyrazolo[1,5-a]pyrazine hydrogen chloride (1/1) (isomer 2)

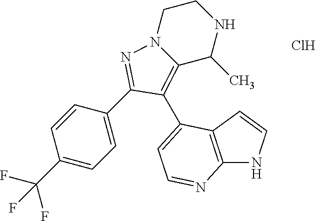 [SMILES: CC1NCCn2nc(-c3ccc(C(F)(F)F)cc3)c(-c3ccnc4[nH]ccc34)c21 ; Cl]

A solution of tert-butyl-4-methyl-3-(1H-pyrrolo[2,3-b]pyridin-4-yl)-2-(4-(trifluoromethyl)phenyl)-6,7-dihydropyrazolo[1,5-a]pyrazine-5(4H)-carboxylate (isomer 2) (500 mg, 1.00 mmol, Intermediate 470) in hydrochloric acid (4 M in ethyl acetate, 5.0 ml) was stirred at 20° C. for 1 hour. The reaction mixture was concentrated under reduced pressure to give 4-methyl-3-(1H-pyrrolo[2,3-b]pyridin-4-yl)-2-(4-(trifluoromethyl)phenyl)-4,5,6,7-tetrahydropyrazolo[1,5-a]pyrazine hydrogen chloride (1/1) (isomer 2) (420 mg, 0.968 mmol, 96% yield) as a brown solid.

LC-MS (Method 16): Rₜ=0.492 min; MS (ESIpos): m/z=398.2 [M+H]⁺

### Intermediate 472

### tert-butyl-3-iodo-4-methyl-2-[4-(trifluoromethyl)phenyl]-6,7-dihydropyrazolo[1,5-a]pyrazine-5(4H)-carboxylate (isomer 1)

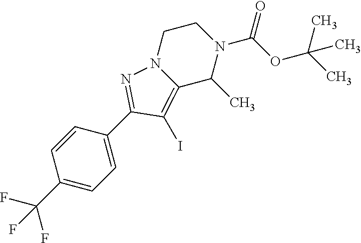 [SMILES: CC1c2c(I)c(-c3ccc(C(F)(F)F)cc3)nn2CCN1C(=O)OC(C)(C)C]

Tert-butyl (4RS)-3-iodo-4-methyl-2-[4-(trifluoromethyl)phenyl]-6,7-dihydropyrazolo[1,5-a]pyrazine-5(4H)-carboxylate (4.00 g, 7.88 mmol, Intermediate 445) was separated by chiral separation [Column: Chiralpak AD-3 50×4.6 mm I.D., 3 μm Mobile phase: Phase A for CO2, and Phase B for IPA (0.05% DEA); Gradient elution: IPA (0.05% DEA) in CO2 from 5% to 40% Flow rate: 3 mL/min; Detector: PDA; Column Temp: 35 C; Back Pressure: 100 Bar] to give tert-butyl-3-iodo-4-methyl-2-[4-(trifluoromethyl)phenyl]-6,7-dihydropyrazolo[1,5-a]pyrazine-5(4H)-carboxylate (isomer 1) (2.20 g, 4.21 mmol, 97% purity, 53% yield) as a white solid.
ee %=93.8%
Optical rotation: [α]_{D}=−76.306° (c=0.815 g/100 ml, methanol)

### Intermediate 473

### tert-butyl-4-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 1)

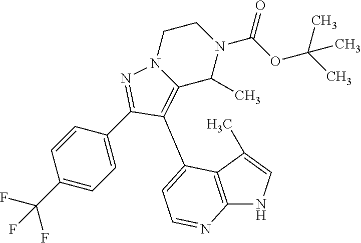 [SMILES: Cc1c[nH]c2nccc(-c3c(-c4ccc(C(F)(F)F)cc4)nn4c3C(C)N(C(=O)OC(C)(C)C)CC4)c12]

To a solution of tert-butyl-3-iodo-4-methyl-2-[4-(trifluoromethyl)phenyl]-6,7-dihydropyrazolo[1,5-a]pyrazine-5(4H)-carboxylate (isomer 1) (500 mg, 0.986 mmol, Intermediate 472) and (3-methyl-1H-pyrrolo[2,3-b]pyridin-4-yl) boronic acid (347 mg, 1.97 mmol, Intermediate 245) in 1,4-dioxane (5.0 ml) and water (1.0 ml) were added sodium carbonate (209 mmol) mg, 1.97 and [1,1-bis(diphenylphosphino) ferrocene]dichloropalladium (II) (72.1 mg, 0.0986 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 1:1) to give tert-butyl-4-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 1) (120 mg, 0.235 mmol, 24% yield) as a yellow oil.

LC-MS (Method 16): Rₜ=0.651 min; MS (ESIpos): m/z=512.4 [M+H]⁺

### Intermediate 474

### 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 1)

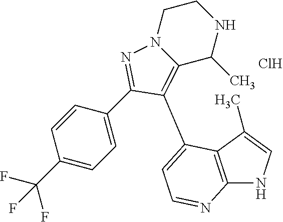 [SMILES: Cc1c[nH]c2nccc(-c3c(-c4ccc(C(F)(F)F)cc4)nn4c3C(C)NCC4)c12 ; Cl]

A solution of tert-butyl-4-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (isomer 1) (120 mg, 0.235 mmol, Intermediate 473) in hydrochloric acid (4 M in ethyl acetate, 3.0 ml) was stirred at 20° C. for 1 hour. The reaction mixture was concentrated under reduced pressure to give 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 1) (100 mg, 0.223 mmol, 95% yield) as a brown solid.

LC-MS (Method 16): Rₜ=0.454 min; MS (ESIpos): m/z=412.2 [M+H]⁺.

### Intermediate 475

### tert-butyl (6R)-6-methyl-2-[4-(trifluoromethyl)phenyl]-6,7-dihydropyrazolo[1,5-a]pyrazine-5(4H)-carboxylate

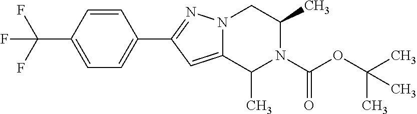 [SMILES: CC1c2cc(-c3ccc(C(F)(F)F)cc3)nn2C[C@@H](C)N1C(=O)OC(C)(C)C]

To a solution of tert-butyl (6R)-2-bromo-6-methyl-6,7-dihydropyrazolo[1,5-a]pyrazine-5(4H)-carboxylate (500 mg, 1.58 mmol, Intermediate 496) and [4-(trifluoromethyl)phenyl]boronic acid (450 mg, 2.37 mmol) in 1,4-dioxane (10 ml) and water (2 ml) were added sodium carbonate (335 mg, 3.16 mmol) and (1,1′-bis(diphenylphosphino) ferrocene) palladium (II) dichloride (116 mg, 158 μmol) at 20° C. and the mixture was stirred at 90° C. for 16 hours under nitrogen atmosphere. The reaction mixture was concentrated under vacuum to give a residue and the residue was purified by silica gel column chromatography (petroleum ether:ethyl acetate=1:0 to 4:1) to give tert-butyl (6R)-6-methyl-2-[4-(trifluoromethyl)phenyl]-6,7-dihydropyrazolo[1,5-a]pyrazine-5(4H)-carboxylate (550 mg, 1.44 mmol, 91% yield) as a white solid.

LC-MS (Method 16): Rₜ=0.960 min; MS (ESIpos): m/z=382.2 [M+H]⁺.

### Intermediate 476

### tert-butyl (6R)-3-iodo-6-methyl-2-[4-(trifluoromethyl)phenyl]-6,7-dihydropyrazolo[1,5-a]pyrazine-5(4H)-carboxylate

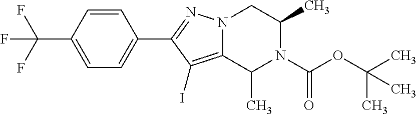 [SMILES: CC1c2c(I)c(-c3ccc(C(F)(F)F)cc3)nn2C[C@@H](C)N1C(=O)OC(C)(C)C]

To a solution of tert-butyl (6R)-6-methyl-2-[4-(trifluoromethyl)phenyl]-6,7-dihydropyrazolo[1,5-a]pyrazine-5(4H)-carboxylate (450 mg, 1.18 mmol, Intermediate 475) in dichloromethane (18 ml) and methanol (9 ml) was added N-iodosuccinimide (531 mg, 2.36 mmol) in portions at 20° C. and the reaction mixture was stirred at 40° C. for 16 hours. The reaction mixture was washed with saturated sodium sulfite aqueous solution and extracted with ethyl acetate. The combined organic layers were dried over anhydrous sodium sulfate, filtered and concentrated under vacuum to give tert-butyl (6R)-3-iodo-6-methyl-2-[4-(trifluoromethyl)phenyl]-6,7-dihydropyrazolo[1,5-a]pyrazine-5(4H)-carboxylate (500 mg, 0.986 mmol, 84% yield) as a white solid.

LC-MS (Method 16): Rₜ=1.114 min; MS (ESIpos): m/z=508.1 [M+H]⁺.

### Intermediate 477

### tert-butyl (6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate

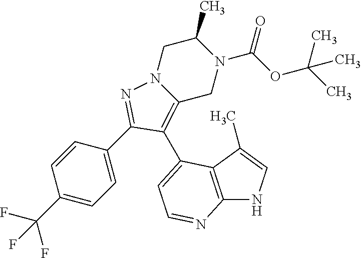 [SMILES: Cc1c[nH]c2nccc(-c3c(-c4ccc(C(F)(F)F)cc4)nn4c3CN(C(=O)OC(C)(C)C)[C@H](C)C4)c12]

To a solution of tert-butyl (6R)-3-iodo-6-methyl-2-[4-(trifluoromethyl)phenyl]-6,7-dihydropyrazolo[1,5-a]pyrazine-5(4H)-carboxylate (600 mg, 1.18 mmol, Intermediate 476) and (3-methyl-1H-pyrrolo[2,3-b]pyridin-4-yl) boronic acid (520 mg, 2.96 mmol, Intermediate 245) in 1,4-dioxane (9 ml) and water (2 ml) were added sodium carbonate (251 mg, 2.37 mmol) and (1,1′-bis(diphenylphosphino) ferrocene) palladium (II) dichloride (71.4 mg, 0.118 mmol) at 20° C. and the mixture was stirred at 80° C. for 16 hours under nitrogen atmosphere. The reaction mixture was concentrated under vacuum to give a residue and the residue was purified by silica gel column chromatography (petroleum ether:ethyl acetate=0:1 to 1:1) to give tert-butyl (6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (230 mg, 0.450 mmol, 38% yield) as a white solid.

LC-MS (Method 16): Rₜ=0.939 min; MS (ESIpos): m/z=512.3 [M+H]⁺.

### Intermediate 478

### (6R)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1)

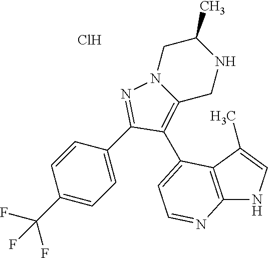 [SMILES: Cc1c[nH]c2nccc(-c3c(-c4ccc(C(F)(F)F)cc4)nn4c3CN[C@H](C)C4)c12 ; Cl]

To a solution of tert-butyl (6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazine-5(4H)-carboxylate (230 mg, 0.450 mmol, Intermediate 477) in ethyl acetate (2 ml) was added hydrochloric acid (4 M in ethyl acetate, 10 ml, 40.0 mmol) at 20° C. and the mixture was stirred at 20° C. for 1 hour. The reaction mixture was concentrated under vacuum to give (6R)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (200 mg, 0.447 mmol, 99% yield) as a yellow oil.

LC-MS (Method 14): Rₜ=0.923 min; MS (ESIpos): m/z=412.2 [M+H]⁺.

### Intermediate 479

### tert-butyl 2-(4-fluorophenyl)-3-iodo-4-methyl-6,7-dihydropyrazolo[1,5-a]pyrazine-5(4H)-carboxylate (isomer 2)

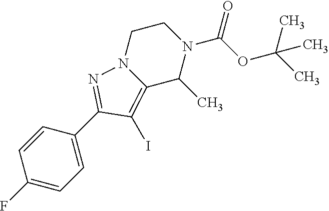 [SMILES: CC1c2c(I)c(-c3ccc(F)cc3)nn2CCN1C(=O)OC(C)(C)C]

The tert-butyl (4RS)-2-(4-fluorophenyl)-3-iodo-4-methyl-6,7-dihydropyrazolo[1,5-a]pyrazine-5(4H)-carboxylate (3.80 g, 8.31 mmol, Intermediate 454) was separated by chiral separation [Column: Chiralcel OJ-3 50×4.6 mm I.D., 3 μm; Mobile phase: Phase A for CO2, and Phase B for IPA (0.05% DEA); Gradient elution: IPA (0.05% DEA) in CO2 from 5% to 40% Flow rate: 3 mL/min; Detector: PDA; Column Temp: 35 C; Back Pressure: 100 Bar] to give tert-butyl 2-(4-fluorophenyl)-3-iodo-4-methyl-6,7-dihydropyrazolo[1,5-a]pyrazine-5(4H)-carboxylate (isomer 2) (1.70 g, 3.62 mmol, 97% purity, 44% yield) as a white solid.
ee %=100%
Optical rotation: [α]_{D}=+96.065° (c=0.364 g/100 ml, methanol)

### Intermediate 480

### tert-butyl 2-(4-fluorophenyl)-4-methyl-3-(1H-pyrrolo[2,3-b]pyridin-4-yl)-6,7-dihydropyrazolo[1,5-a]pyrazine-5(4H)-carboxylate (isomer 2)

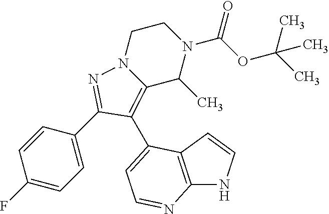 [SMILES: CC1c2c(-c3ccnc4[nH]ccc34)c(-c3ccc(F)cc3)nn2CCN1C(=O)OC(C)(C)C]

To a solution of tert-butyl 2-(4-fluorophenyl)-3-iodo-4-methyl-6,7-dihydropyrazolo[1,5-a]pyrazine-5(4H)-carboxylate (isomer 2) (600 mg, 1.31 mmol, Intermediate 479) and 1H-pyrrolo[2,3-b]pyridin-4-ylboronic acid (425 mg, 2.62 mmol) in 1,4-dioxane (5.0 ml) and water (1.0 ml) were added sodium carbonate (278 mg, 2.62 mmol) and [1,1-bis(diphenylphosphino) ferrocene]dichloropalladium (II) (96.0 mg, 0.131 mmol) at 20° C. The mixture was stirred at 80° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 3:2) to give tert-butyl 2-(4-fluorophenyl)-4-methyl-3-(1H-pyrrolo[2,3-b]pyridin-4-yl)-6,7-dihydropyrazolo[1,5-a]pyrazine-5(4H)-carboxylate (isomer 2) (400 mg, 0.894 mmol, 68% yield) as a yellow oil.

LC-MS (Method 16): Rₜ=0.891 min; MS (ESIpos): m/z=448.2 [M+H]⁺.

### Intermediate 481

### 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 2)

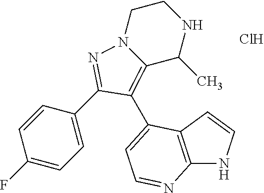 [SMILES: CC1NCCn2nc(-c3ccc(F)cc3)c(-c3ccnc4[nH]ccc34)c21 ; Cl]

A solution of tert-butyl 2-(4-fluorophenyl)-4-methyl-3-(1H-pyrrolo[2,3-b]pyridin-4-yl)-6,7-dihydropyrazolo[1,5-a]pyrazine-5(4H)-carboxylate (isomer 2) (400 mg, 0.894 mmol, Intermediate 480) in hydrochloric acid (4.0 M in ethyl acetate, 10 ml, 40.0 mmol) was stirred at 20° C. for 1 hour. The reaction mixture was concentrated under reduced pressure to give 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 2) (350 mg, 0.912 mmol, 102% yield) as a brown solid.

LC-MS (Method 16): Rₜ=0.389 min; MS (ESIpos): m/z=348.1 [(M−36)+H]⁺

### Intermediate 482

### tert-butyl-2-(4-fluorophenyl)-4-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (isomer 1)

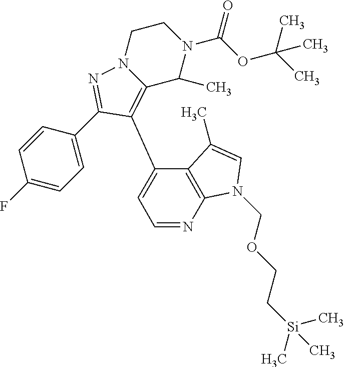 [SMILES: Cc1cn(COCC[Si](C)(C)C)c2nccc(-c3c(-c4ccc(F)cc4)nn4c3C(C)N(C(=O)OC(C)(C)C)CC4)c12]

To a solution of tert-butyl-2-(4-fluorophenyl)-3-iodo-4-methyl-6,7-dihydropyrazolo[1,5-a]pyrazine-5(4H)-carboxylate (isomer 2) (1.00 g, 2.19 mmol, Intermediate 479) and 3-methyl-4-(4,4,5,5-tetramethyl-1,3,2-dioxaborolan-2-yl)-1-{[2-(trimethylsilyl) ethoxy]methyl}-1H-pyrrolo[2,3-b]pyridine (1.27 g, 3.28 mmol, Intermediate 254) in 1,4-dioxane (20 ml) and water (4.0 ml) were added sodium carbonate (464 mg, 4.37 mmol) and [1,1-bis(diphenylphosphino) ferrocene]dichloropalladium (II) (160 mg, 0.219 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 1:1) to give tert-butyl-2-(4-fluorophenyl)-4-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (isomer 1) (500 mg, 0.845 mmol, 39% yield) as a yellow oil.

LC-MS (Method 16): Rₜ=1.159 min; MS (ESIpos): m/z=592.4 [M+H]⁺

### Intermediate 483

### 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 1)

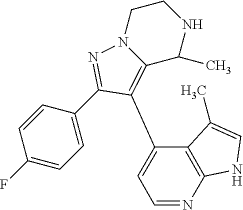 [SMILES: Cc1c[nH]c2nccc(-c3c(-c4ccc(F)cc4)nn4c3C(C)NCC4)c12]

To a solution of tert-butyl-2-(4-fluorophenyl)-4-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (isomer 2) (600 mg, 1.01 mmol, Intermediate 482) in dichloromethane (6.0 ml) was added trifluoroacetic acid (2.0 ml, 26.0 mmol) at 20° C. The mixture was stirred at 20° C. for 16 hours. The reaction mixture was concentrated under reduced pressure to give a residue. The residue was dissolved in methanol (5 ml), followed by addition of potassium carbonate (279 mg, 2.02 mmol) in water (5 ml) and the resulting mixture was stirred at 20° C. for 3 hours. The reaction solution was poured into water and extracted with ethyl acetate. The combined organic layers were washed with brine, dried over anhydrous sodium sulfate, filtered and concentrated in vacuo to give the title compound (200 mg, 0.553 mmol, 55% yield) as a brown solid.

LC-MS (Method 16): Rₜ=0.803 min; MS (ESIpos): m/z=362.2 [M+H]⁺.

### Intermediate 484

### tert-butyl (6R)-2-(4-fluorophenyl)-6-methyl-6,7-dihydropyrazolo[1,5-a]pyrazine-5(4H)-carboxylate

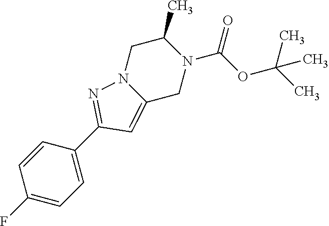 [SMILES: C[C@@H]1Cn2nc(-c3ccc(F)cc3)cc2CN1C(=O)OC(C)(C)C]

To a solution of tert-butyl (6R)-2-bromo-6-methyl-6,7-dihydropyrazolo[1,5-a]pyrazine-5(4H)-carboxylate (500 mg, 1.58 mmol, Intermediate 496) and (4-fluorophenyl) boronic acid (332 mg, 2.37 mmol) in 1,4-dioxane (10 ml) and water (2 ml) were added sodium carbonate (335 mg, 3.16 mmol) and (1,1′-bis(diphenylphosphino) ferrocene) palladium (II) dichloride (116 mg, 0.158 mmol at 20° C. and the mixture was stirred at 90° C. for 16 hours under nitrogen atmosphere. The reaction mixture was concentrated under vacuum to give a residue and the residue was purified by silica gel column chromatography (petroleum ether:ethyl acetate=1:0 to 4:1) to give tert-butyl (6R)-2-(4-fluorophenyl)-6-methyl-6,7-dihydropyrazolo[1,5-a]pyrazine-5(4H)-carboxylate (500 mg, 1.51 mmol, 95% yield) as a white solid.

LC-MS (Method 16): Rₜ=1.005 min; MS (ESIpos): m/z=332.2 [M+H]⁺.

### Intermediate 485

### tert-butyl (6R)-2-(4-fluorophenyl)-3-iodo-6-methyl-6,7-dihydropyrazolo[1,5-a]pyrazine-5(4H)-carboxylate

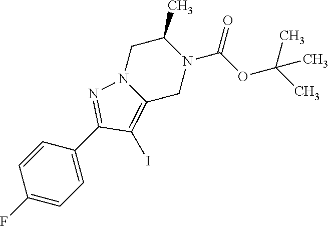 [SMILES: C[C@@H]1Cn2nc(-c3ccc(F)cc3)c(I)c2CN1C(=O)OC(C)(C)C]

To a solution of tert-butyl (6R)-2-(4-fluorophenyl)-6-methyl-6,7-dihydropyrazolo[1,5-a]pyrazine-5(4H)-carboxylate (500 mg, 1.51 mmol, Intermediate 484) in dichloromethane (10 ml) and methanol (5 ml) was added N-iodosuccinimide (509 mg, 2.26 mmol) in portions at 20° C. and the mixture was stirred at 45° C. for 16 hours. The reaction mixture was washed with saturated sodium sulfite aqueous solution and extracted with ethyl acetate. The combined organic layers were dried over anhydrous sodium sulfate, filtered and concentrated under vacuum to give tert-butyl (6R)-2-(4-fluorophenyl)-3-iodo-6-methyl-6,7-dihydropyrazolo[1,5-a]pyrazine-5(4H)-carboxylate (600 mg, 1.31 mmol, 87% yield) as a white solid.

LC-MS (Method 16): Rₜ=1.093 min; MS (ESIpos): m/z=458.1 [M+H]⁺

### Intermediate 486

### tert-butyl (6R)-2-(4-fluorophenyl)-6-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate

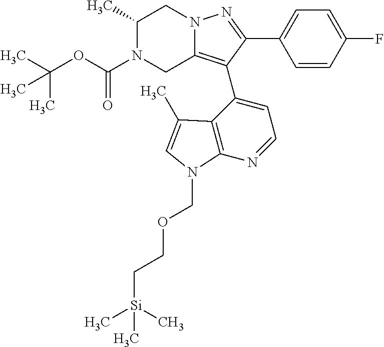 [SMILES: Cc1cn(COCC[Si](C)(C)C)c2nccc(-c3c(-c4ccc(F)cc4)nn4c3CN(C(=O)OC(C)(C)C)[C@H](C)C4)c12]

To a solution of tert-butyl (6R)-2-(4-fluorophenyl)-3-iodo-6-methyl-6,7-dihydropyrazolo[1,5-a]pyrazine-5(4H)-carboxylate (550 mg, 1.20 mmol, Intermediate 485) and 3-methyl-4-(4,4,5,5-tetramethyl-1,3,2-dioxaborolan-2-yl)-1-{[2-(trimethylsilyl) ethoxy]methyl}-1H-pyrrolo[2,3-b]pyridine (1.17 g, 3.01 mmol, Intermediate 254) in 1,4-dioxane (22 ml) and water (5 ml) were added sodium carbonate (255 mg, 2.41 mmol) and (1,1′-bis(diphenylphosphino) ferrocene) palladium (II) dichloride (88.0 mg, 0.120 mmol) at 20° C. and the mixture was stirred at 90° C. for 16 hours under nitrogen atmosphere. The reaction mixture was concentrated under vacuum to give a residue and the residue was purified by silica gel column chromatography (petroleum ether:ethyl acetate=1:0 to 3:1) to give tert-butyl (6R)-2-(4-fluorophenyl)-6-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (700 mg, 1.18 mmol, 98% yield) as a brown oil.

LC-MS (Method 16): Rₜ=1.177 min; MS (ESIpos): m/z=592.4 [M+H]⁺.

### Intermediate 487

### (6R)-2-(4-fluorophenyl)-6-methyl-3-(3-methyl-1H-pyrrolo[2,3-b]pyridin-4-yl)-4,5,6,7-tetrahydropyrazolo[1,5-a]pyrazine

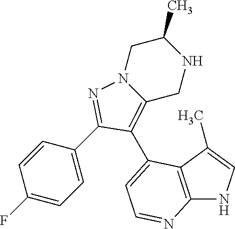 [SMILES: Cc1c[nH]c2nccc(-c3c(-c4ccc(F)cc4)nn4c3CN[C@H](C)C4)c12]

A solution of tert-butyl (6R)-2-(4-fluorophenyl)-6-methyl-3-(3-methyl-1-{[2-(trimethylsilyl) ethoxy]methyl}-1H-pyrrolo[2,3-b]pyridin-4-yl)-6,7-dihydropyrazolo[1,5-a]pyrazine-5(4H)-carboxylate (600 mg, 1.01 mmol, Intermediate 486) in dichloromethane (9 ml) and frifluoroacetic acid (3 ml, 38.9 mmol) was stirred at 20° C. for 16 hours. The reaction mixture was concentrated under vacuum to give a residue. The residue was dissolved in ethyl acetate, the resulting solution was washed with saturated sodium carbonate aqueous solution and concentrated under vacuum to give a residue. The residue was dissolved in methanol (5 ml), followed by addition of potassium carbonate (279 mg, 2.02 mmol) in water (5 ml) at 20° C. The suspension was stirred at 20° C. for 16 hours. The suspension was poured into water and the resulting mixture was extracted with ethyl acetate. Combined extracts were dried over anhydrous sodium sulfate, filtered and concentrated under vacuum to give (6R)-2-(4-fluorophenyl)-6-methyl-3-(3-methyl-1H-pyrrolo[2,3-b]pyridin-4-yl)-4,5,6,7-tetrahydropyrazolo[1,5-a]pyrazine (300 mg, 0.830 mmol, 82% yield) as a brown oil.

LC-MS (Method 16): Rₜ=0.821 min; MS (ESIpos): m/z=362.2 [M+H]⁺.

### Intermediate 488

### methyl 3-bromo-1-{(2S)-2-[(tert-butoxycarbonyl)amino]propyl}-1H-pyrazole-5-carboxylate

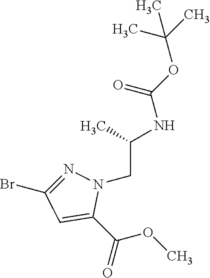 [SMILES: COC(=O)c1cc(Br)nn1C[C@H](C)NC(=O)OC(C)(C)C]

To a solution of methyl 3-bromo-1H-pyrazole-5-carboxylate (20.0 g, 97.6 mmol) in tetrahydrofuran (200 ml) were added tert-butyl [(2S)-1-hydroxypropan-2-yl]carbamate (17.1 g, 97.6 mmol), triphenylphosphine (51.2 g, 195 mmol) and diisopropyl azodicarboxylate (39.5 g, 195 mmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=5:1) to give methyl 3-bromo-1-{(2S)-2-[(tert-butoxycarbonyl)amino]propyl}-1H-pyrazole-5-carboxylate (42.0 g, 80% purity, 95% yield) as a pink crystal.

LC-MS (Method 14): Rₜ=0.877 min; MS (ESIpos): m/z=262.0 [(M−100)+H]⁺.

¹H NMR (400 MHz, DMSO-d₆), δ [ppm]=6.79 (s, 1H), 5.08-5.00 (m, 1H), 4.75 (d, J=6.8 Hz, 1H), 4.64-4.39 (m, 2H), 3.87 (s, 3H), 1.34 (s, 9H), 1.14 (d, J=6.8 Hz, 3H).

### Intermediate 489

### tert-butyl {(2S)-1-[3-bromo-5-(hydroxymethyl)-1H-pyrazol-1-yl]propan-2-yl}carbamate

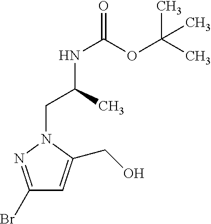 [SMILES: C[C@@H](Cn1nc(Br)cc1CO)NC(=O)OC(C)(C)C]

To a solution of methyl 3-bromo-1-{(2S)-2-[(tert-butoxycarbonyl)amino]propyl}-1H-pyrazole-5-carboxylate (42.0 g, 80% purity, 92.8 mmol Intermediate 487) in ethanol (420 ml) were added sodium tetrahydroborate (10.5 g, 278 mmol) at 0° C. under nitrogen atmosphere. The mixture was stirred at 25° C. for 16 hours. The reaction was quenched with water and the resulting mixture was extracted with ethyl acetate. The combined organic phase was washed with brine, dried over anhydrous sodium sulfate, filtered and concentrated in vacuo to give tert-butyl {(2S)-1-[3-bromo-5-(hydroxymethyl)-1H-pyrazol-1-yl]propan-2-yl}carbamate (33.4 g, 91% purity, 98% yield) as a colourless oil.

LC-MS (Method 14): Rₜ=0.779 min; MS (ESIpos): m/z=236.0 [(M−100)+H]⁺

¹H NMR (400 MHz, DMSO-d₆), δ [ppm]=6.24 (s, 1H), 5.12-4.94 (m, 1H), 4.70-4.59 (m, 2H), 4.12-4.06 (m, 1H), 1.38 (s, 9H), 1.24 (d, J=6.0 Hz, 3H).

### Intermediate 490

### N-[(3-bromo-1-{(2S)-2-[(tert-butoxycarbonyl)amino]propyl}-1H-pyrazol-5-yl)methyl]-N,N-diethylethanaminium methanesulfonate

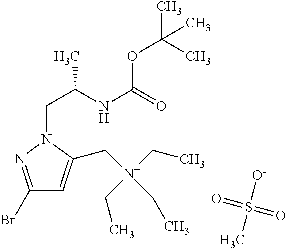

To a solution of tert-butyl {(2S)-1-[3-bromo-5-(hydroxymethyl)-1H-pyrazol-1-yl]propan-2-yl}carbamate (22.4 g, 91% purity, 61.0 mmol Intermediate 489) in dichloromethane (220 ml) was added triethylamine (51 ml, 370 mmol) and methanesulfonic anhydride (21.3 g, 122 mmol) at 0° C. under nitrogen atmosphere. The mixture was stirred at 25° C. for 16 hours. The reaction mixture was concentrated in vacuo to give N-[(3-bromo-1-{(2S)-2-[(tert-butoxycarbonyl)amino]propyl}-1H-pyrazol-5-yl)methyl]-N,N-diethylethanaminium methanesulfonate (31.0 g, 50.1 mmol, 82% yield) as a brown oil.

LC-MS (Method 14): Rₜ=0.702 min; MS (ESIpos): m/z=419.1 [(M−95)+H]⁺.

### Intermediate 491

### tert-butyl(S)-2-bromo-6-methyl-6,7-dihydropyrazolo[1,5-a]pyrazine-5(4H)-carboxylate

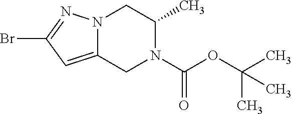 [SMILES: C[C@H]1Cn2nc(Br)cc2CN1C(=O)OC(C)(C)C]

To a solution of N-[(3-bromo-1-{(2S)-2-[(tert-butoxycarbonyl)amino]propyl}-1H-pyrazol-5-yl)methyl]-N,N-diethylethanaminium methanesulfonate (31.0 g, 83% purity, 50.1 mmol, Intermediate 490) in N,N-dimethylformamide (250 ml) and tetrahydrofuran (50 ml) was added sodium hydride (16.0 g, 60% purity, 401 mmol) at 0° C. under nitrogen atmosphere. The mixture was stirred at 25° C. for 16 hours. The reaction was quenched with water and the resulting solution was extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=5:1) to give methyl tert-butyl(S)-2-bromo-6-methyl-6,7-dihydropyrazolo[1,5-a]pyrazine-5(4H)-carboxylate (20.0 g, 95% purity, 120% yield) as a white solid.

LC-MS (Method 15): Rₜ=0.951 min; MS (ESIpos): m/z=318.0 [M+H]⁺.

¹H NMR (400 MHz, DMSO-d6), δ [ppm]=6.09 (s, 1H), 4.92 (d, J=17.2 Hz, 1H), 4.79 (s, 1H), 4.30-4.15 (m, 2H), 4.05-4.00 (m, 1H), 1.48 (s, 9H), 1.18 (d, J=6.8 Hz, 3H).

Optical rotation: [α]_{D}=−42.8° (c=1.0706 g/100 ml, methanol)

### Intermediate 492

### tert-butyl (6S)-2-bromo-3-iodo-6-methyl-6,7-dihydropyrazolo[1,5-a]pyrazine-5(4H)-carboxylate

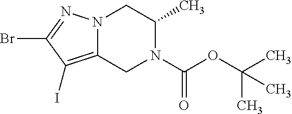 [SMILES: C[C@H]1Cn2nc(Br)c(I)c2CN1C(=O)OC(C)(C)C]

To a solution of tert-butyl (6S)-2-bromo-6-methyl-6,7-dihydropyrazolo[1,5-a]pyrazine-5(4H)-carboxylate (10.0 g, 95% purity, 30.0 mmol, Intermediate 491) in dichloromethane (30 ml) and methanol (70 ml) was added N-iodosuccinimide (13.5 g, 60.1 mmol) at 25° C. The mixture was stirred at 45° C. for 16 hours. The reaction mixture was poured into saturated sodium thiosulfate aqueous solution and extracted with ethyl acetate. The combined organic phase was washed with birne, dried over anhydrous sodium sulfate, filtered and concentrated in vacuo to give a residue. The residue was purified by column chromatography on silica gel (100~200 mesh, petroleum ether:ethyl acetate=20:1 to 10:1) to give methyl tert-butyl(S)-2-bromo-3-iodo-6-methyl-6,7-dihydropyrazolo[1,5-a]pyrazine-5(4H)-carboxylate (12.8 g, 89% purity, 54% yield) as a white solid.

LC-MS (Method 14): Rₜ=0.945 min; MS (ESIpos): m/z=441.9 [M+H]⁺.

¹H NMR (400 MHz, DMSO-d6), δ [ppm]=4.63 (d, J=17.2 Hz, 2H), 4.22-4.14 (m, 1H), 4.12 (d, J=8.0 Hz, 1H), 4.07-4.02 (m, 1H), 1.44 (s, 9H), 1.07 (d, J=6.8 Hz, 3H).

Optical rotation: [α]_{D}=−72.8° ° (c=0.8380 g/100 ml, methanol)

### Intermediate 493

### methyl 3-bromo-1-{(2R)-2-[(tert-butoxycarbonyl)amino]propyl}-1H-pyrazole-5-carboxylate

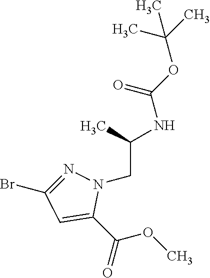 [SMILES: COC(=O)c1cc(Br)nn1C[C@@H](C)NC(=O)OC(C)(C)C]

To a solution of methyl 3-bromo-1H-pyrazole-5-carboxylate (20.0 g, 97.6 mmol) in tetrahydrofuran (300 ml) were added tert-butyl [(2R)-1-hydroxypropan-2-yl]carbamate (17.1 g. 97.6 mmol), triphenylphosphine (51.2 g, 195 mmol) and diisopropyl azodicarboxylate (39.5 g, 195 mmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=5:1) to give methyl 3-bromo-1-{(2R)-2-[(tert-butoxycarbonyl)amino]propyl}-1H-pyrazole-5-carboxylate (37.0 g, 95% purity, 99% yield) as a pink crystal.

LC-MS (Method 16): Rₜ=0.905 min; MS (ESIpos): m/z=264.0 [M+H]⁺.

¹H NMR (400 MHz, DMSO-d₆), δ [ppm]=6.82 (s, 1H), 5.14-5.03 (m, 1H), 4.80-4.65 (m, 1H), 4.64-4.37 (m, 2H), 3.90 (s, 3H), 1.36 (s, 9H), 1.17 (d, J=6.8 Hz, 3H).

### Intermediate 494

### tert-butyl {(2R)-1-[3-bromo-5-(hydroxymethyl)-1H-pyrazol-1-yl]propan-2-yl}carbamate

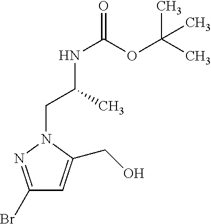 [SMILES: C[C@H](Cn1nc(Br)cc1CO)NC(=O)OC(C)(C)C]

To a solution of methyl 3-bromo-1-{(2R)-2-[(tert-butoxycarbonyl)amino]propyl}-1H-pyrazole-5-carboxylate (37.0 g, 95% purity, 97.0 mmol, in ethanol (380 ml) were added sodium tetrahydroborate (11.0 g, 291 mmol) at 0° C. under nitrogen atmosphere. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give tert-butyl {(2R)-1-[3-bromo-5-(hydroxymethyl)-1H-pyrazol-1-yl]propan-2-yl}carbamate (31.0 g, 94% purity, 90% yield) as a colourless oil.

LC-MS (Method 14): Rₜ=0.792 min; MS (ESIpos): m/z=236.0 [M+H]⁺.

¹H NMR (400 MHz, DMSO-d6), δ [ppm]=6.24 (s, 1H), 5.10-4.90 (m, 1H), 4.64 (d, J=6.4 Hz, 2H), 4.12-4.08 (m, 1H), 1.38 (s, 9H), 1.27 (d, J=6.4 Hz, 3H).

### Intermediate 495

### N-[(3-bromo-1-{(2R)-2-[(tert-butoxycarbonyl)amino]propyl}-1H-pyrazol-5-yl)methyl]-N,N-diethylethanaminium methanesulfonate

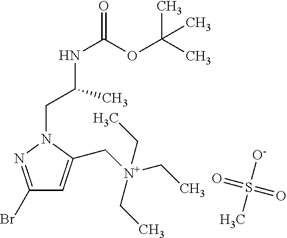

To a solution of tert-butyl {(2R)-1-[3-bromo-5-(hydroxymethyl)-1H-pyrazol-1-yl]propan-2-yl}carbamate (21.0 g, 94% purity, 59.1 mmol, Intermediate 494) in dichloromethane (210 ml) were added triethylamine (49 ml, 350 mmol) and methanesulfonic anhydride (20.6 g, 118 mmol) at 0° C. The mixture was stirred at 25° C. for 16 hours. The reaction mixture was concentrated in vacuo to give (R)—N-((3-bromo-1-(2-((tert-butoxycarbonyl)amino) propyl)-1H-pyrazol-5-yl)methyl)-N,N-diethylethanaminium (31.0 g, 85% purity, 87% yield) as a brown oil.

LC-MS (Method 14): Rₜ=0.611 min; MS (ESIpos): m/z=419.1 [M+H]⁺.

### Intermediate 496

### tert-butyl (6R)-2-bromo-6-methyl-6,7-dihydropyrazolo[1,5-a]pyrazine-5(4H)-carboxylate

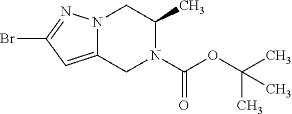 [SMILES: C[C@@H]1Cn2nc(Br)cc2CN1C(=O)OC(C)(C)C]

To a solution of N-[(3-bromo-1-{(2R)-2-[(tert-butoxycarbonyl)amino]propyl}-1H-pyrazol-5-yl)methyl]-N,N-diethylethanaminium methanesulfonate (14.0 g, 84% purity, 22.9 mmol, Intermediate 495) in N,N-dimethylformamide (120 ml) and tetrahydrofuran (30 ml) was added sodium hydride (7.33 g, 60% purity, 183 mmol) at 0° C. under nitrogen atmosphere. The mixture was stirred at 25° C. for 16 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=5:1) to give tert-butyl (6R)-2-bromo-6-methyl-6,7-dihydropyrazolo[1,5-a]pyrazine-5(4H)-carboxylate (19.0 g, 92% purity, 107% yield) as a white solid.

LC-MS (Method 14): Rₜ=0.878 min; MS (ESIpos): m/z=316.0 [M+H]⁺.

¹H NMR (400 MHz, DMSO-da), δ [ppm]=6.10 (s, 1H), 4.94 (d, J=17.2 Hz, 1H), 4.81 (s, 1H), 4.30-4.15 (m, 2H), 4.09-4.01 (m, 1H), 1.50 (s, 9H), 1.20 (d, J=7.2 Hz, 3H).

Optical rotation: [α]_{D}=+42.0° (c=0.6334 g/100 ml, methanol)

### Intermediate 497

### tert-butyl (6R)-2-bromo-3-iodo-6-methyl-6,7-dihydropyrazolo[1,5-a]pyrazine-5(4H)-carboxylate

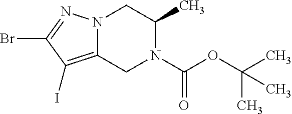 [SMILES: C[C@@H]1Cn2nc(Br)c(I)c2CN1C(=O)OC(C)(C)C]

To a solution of tert-butyl (6R)-2-bromo-6-methyl-6,7-dihydropyrazolo[1,5-a]pyrazine-5(4H)-carboxylate (10.0 g, 92% purity, 29.1 mmol, Intermediate 496) in dichloromethane (30 ml) and methanol (70 ml) was added N-iodosuccinimide (13.1 g, 58.2 mmol) at 25° C. The mixture was stirred at 45° C. for 16 hours. The reaction mixture was poured into saturated sodium thiosulfate aqueous solution and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=20:1 to 10:

1) to give tert-butyl (6R)-2-bromo-3-iodo-6-methyl-6,7-dihydropyrazolo[1,5-a]pyrazine-5(4H)-carboxylate (12.6 g, 97% purity, 60% yield) as a white solid.

LC-MS (Method 14): Rₜ=0.949 min; MS (ESIpos): m/z=441.9 [M+H]⁺.

¹H NMR (400 MHz, DMSO-d₆), δ [ppm]=4.63 (d, J=17.6 Hz, 2H), 4.21-4.15 (m, 1H), 4.12 (d, J=17.6 Hz, 1H), 4.07-4.02 (m, 1H), 1.44 (s, 9H), 1.07 (d, J=6.8 Hz, 3H).

Optical rotation: [α]_{D}=+73.9° (c=0.4058 g/100 ml, methanol)

### Intermediate 498

### tert-butyl (6S)-2-bromo-6-methyl-3-(pyridin-4-yl)-6,7-dihydropyrazolo[1,5-a]pyrazine-5(4H)-carboxylate

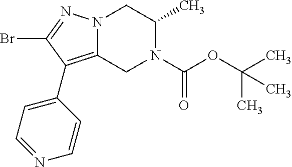 [SMILES: C[C@H]1Cn2nc(Br)c(-c3ccncc3)c2CN1C(=O)OC(C)(C)C]

To a solution of tert-butyl (6S)-2-bromo-3-iodo-6-methyl-6,7-dihydropyrazolo[1,5-a]pyrazine-5(4H)-carboxylate (1.00 g, 89% purity, 2.01 mmol, Intermediate 492) and pyridin-4-ylboronic acid (247 mg, 2.01 mmol) in 1,4-dioxane (10 ml) and water (1.0 ml) were added sodium carbonate (533 5.03 mmol) mg, and [1,1-bis(diphenylphosphino) ferrocene]palladium (II) chloride (147 mg, 0.201 mmol) at 25° C. The mixture was stirred at 80° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give tert-butyl (6S)-2-bromo-6-methyl-3-(pyridin-4-yl)-6,7-dihydropyrazolo[1,5-a]pyrazine-5(4H)-carboxylate (690 mg, 89% purity, 78% yield) as a colourless oil.

LC-MS (Method 17): Rₜ=0.462 min; MS (ESIpos): m/z=395.0 [M+H]⁺.

### Intermediate 499

### tert-butyl (6S)-2-(4-chloro-2-fluorophenyl)-6-methyl-3-(pyridin-4-yl)-6,7-dihydropyrazolo[1,5-a]pyrazine-5(4H)-carboxylate

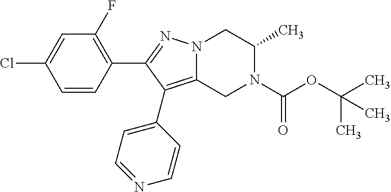 [SMILES: C[C@H]1Cn2nc(-c3ccc(Cl)cc3F)c(-c3ccncc3)c2CN1C(=O)OC(C)(C)C]

To a solution of tert-butyl (6S)-2-bromo-6-methyl-3-(pyridin-4-yl)-6,7-dihydropyrazolo[1,5-a]pyrazine-5(4H)-carboxylate (300 mg, 89% purity, 0.679 mmol, Intermediate 498) and (4-chloro-2-fluorophenyl) boronic acid (178 mg, 1.02 mmol) in 1,4-dioxane (4.0 ml) and water (1.0 ml) were added sodium carbonate (144 mg, 1.36 mmol) and [1,1-bis(diphenylphosphino) ferrocene]palladium (II) chloride (49.7 mg, 0.0679 mmol) at 25° C. The mixture was stirred at 90° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give tert-butyl (6S)-2-(4-chloro-2-fluorophenyl)-6-methyl-3-(pyridin-4-yl)-6,7-dihydropyrazolo[1,5-a]pyrazine-5(4H)-carboxylate (250 mg, 78% purity, 65% yield) as a colourless oil.

LC-MS (Method 17): Rₜ=0.522 min; MS (ESIpos): m/z=443.1 [M+H]⁺.

### Intermediate 500

### (6S)-2-(4-chloro-2-fluorophenyl)-6-methyl-3-(pyridin-4-yl)-4,5,6,7-tetrahydropyrazolo[1,5-a]pyrazine hydrogen chloride (1/1)

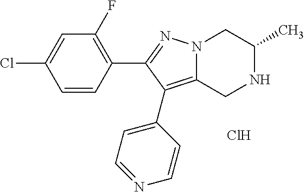 [SMILES: C[C@H]1Cn2nc(-c3ccc(Cl)cc3F)c(-c3ccncc3)c2CN1 ; Cl]

The solution of tert-butyl (6S)-2-(4-chloro-2-fluorophenyl)-6-methyl-3-(pyridin-4-yl)-6,7-dihydropyrazolo[1,5-a]pyrazine-5(4H)-carboxylate (250 mg, 78% purity, 0.440 mmol, Intermediate 499) in hydrochloric acid (4.0 M in dioxane, 3.0 ml, 12.0 mmol) was stirred at 25° C. for 3 hours. The reaction mixture was concentrated under reduced pressure to give (6S)-2-(4-chloro-2-fluorophenyl)-6-methyl-3-(pyridin-4-yl)-4,5,6,7-tetrahydropyrazolo[1,5-a]pyrazine hydrogen chloride (1/1) (200 mg, 76% purity, 91% yield) as a colorless oil.

LC-MS (Method 14): Rₜ=0.242 min; MS (ESIpos): m/z=343.0 [(M−36)+H]⁺.

### Intermediate 501

### tert-butyl (6R)-2-bromo-6-methyl-3-(pyridin-4-yl)-6,7-dihydropyrazolo[1,5-a]pyrazine-5(4H)-carboxylate

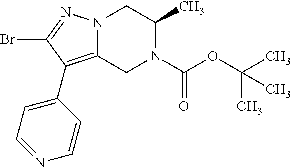 [SMILES: C[C@@H]1Cn2nc(Br)c(-c3ccncc3)c2CN1C(=O)OC(C)(C)C]

To a solution of tert-butyl (6R)-2-bromo-3-iodo-6-methyl-6,7-dihydropyrazolo[1,5-a]pyrazine-5(4H)-carboxylate (1.00 g, 97% purity, 2.19 mmol, Intermediate 497) and pyridin-4-ylboronic acid (270 mg, 2.19 mmol) in 1,4-dioxane (10 ml) and water (1.0 ml) were added sodium carbonate (581 mg, 5.49 mmol) and [1,1-bis(diphenylphosphino) ferrocene]palladium (II) chloride (161 mg, 0.219 mmol) at 25° C. The mixture was stirred at 80° C. for 16 hours under nitrogen atmosphere. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petroleum ether:ethyl acetate=10:1 to 1:1) to give tert-butyl (6R)-2-bromo-6-methyl-3-(pyridin-4-yl)-6,7-dihydropyrazolo[1,5-a]pyrazine-5(4H)-carboxylate (650 mg, 89% purity, 67% yield) as a colourless oil.

LC-MS (Method 17): Rₜ=0.483 min; MS (ESIpos): m/z=393.0 [M+H]⁺.

### Intermediate 502

### tert-butyl (6R)-2-(4-chloro-2-fluorophenyl)-6-methyl-3-(pyridin-4-yl)-6,7-dihydropyrazolo[1,5-a]pyrazine-5(4H)-carboxylate

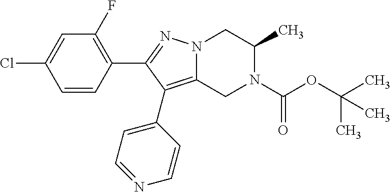 [SMILES: C[C@@H]1Cn2nc(-c3ccc(Cl)cc3F)c(-c3ccncc3)c2CN1C(=O)OC(C)(C)C]

To a solution of tert-butyl (6R)-2-bromo-6-methyl-3-(pyridin-4-yl)-6,7-dihydropyrazolo[1,5-a]pyrazine-5(4H)-carboxylate (300 mg, 89% purity, 0.679 mmol, Intermediate 501) and (4-chloro-2-fluorophenyl) boronic acid (178 mg, 1.02 mmol) in 1,4-dioxane (4.0 ml) and water (1.0 ml) was added sodium carbonate (144 mg, 1.36 mmol) and [1,1-bis(diphenylphosphino) ferrocene]palladium (II) chloride (49.7 mg, 0.0679 mmol) at 25° C. The mixture was stirred at 90° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (100~200 mesh, petreoleum ether:ethyl acetate=10:1 to 1:1) to give tert-butyl (6R)-2-(4-chloro-2-fluorophenyl)-6-methyl-3-(pyridin-4-yl)-6,7-dihydropyrazolo[1,5-a]pyrazine-5(4H)-carboxylate (269 mg, 80% purity, 72% yield) as a colourless oil.

LC-MS (Method 17): Rₜ=0.525 min; MS (ESIpos): m/z=443.1 [M+H]⁺.

### Intermediate 503

### (6R)-2-(4-chloro-2-fluorophenyl)-6-methyl-3-(pyridin-4-yl)-4,5,6,7-tetrahydropyrazolo[1,5-a]pyrazine hydrogen chloride (1/1)

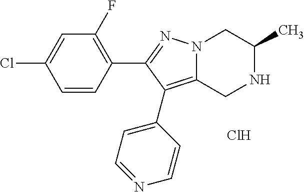 [SMILES: C[C@@H]1Cn2nc(-c3ccc(Cl)cc3F)c(-c3ccncc3)c2CN1 ; Cl]

The solution of tert-butyl (6R)-2-(4-chloro-2-fluorophenyl)-6-methyl-3-(pyridin-4-yl)-6,7-dihydropyrazolo[1,5-a]pyrazine-5(4H)-carboxylate (267 mg, 80% purity, 0.482 mmol, Intermediate 502) in hydrochloric acid (4.0 M in dioxane, 3.0 ml, 12.0 mmol) was stirred at 25° C. for 3 hours. The reaction mixture was concentrated under reduced pressure to give (6R)-2-(4-chloro-2-fluorophenyl)-6-methyl-3-(pyridin-4-yl)-4,5,6,7-tetrahydropyrazolo[1,5-a]pyrazine hydrogen chloride (1/1) (200 mg, 81% purity, 89% yield) as a colorless oil.

LC-MS (Method 14): Rₜ=0.259 min; MS (ESIpos): m/z=343.0 [(M−36)+H]⁺.

### Intermediate 504

### tert-butyl 2-anilino-6,7-dihydropyrazolo[1,5-a]pyrazine-5(4H)-carboxylate

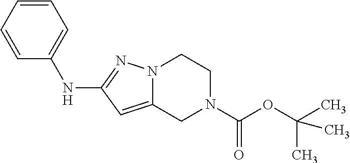 [SMILES: CC(C)(C)OC(=O)N1CCn2nc(Nc3ccccc3)cc2C1]

To a solution of tert-butyl 2-amino-6,7-dihydropyrazolo[1,5-a]pyrazine-5(4H)-carboxylate (500 mg, 2.10 mmol) in 2 ml of 1,4-dioxane, bromobenzene (440 μl, 4.2 mmol; CAS-RN: [108-86-1]) was added. The resulting mixture was purged with argon for 5 min. Potassium carbonate (580 mg, 4.20 mmol), tris-(dibenzylidenacetone) dipalladium (76.9 mg, 83.9 μmol) and Xphos (40.0 mg, 83.9 μmol; CAS-RN: [564483-18-7]) were added and the mixture was heated to 100° C. for 18 h. After cooling to rt, 2 ml of water were added. The mixture was diluted with 5 ml of ethyl acetate and filtered through a pad of celite. The layers were separated and the aqueous layer was extracted with ethyl acetate. The combined organic layers were dried over sodium sulfate, filtered and the solvent was evaporated under reduced pressure. The residue was purified by silica gel column chromatography (eluent: ethyl acetate/cyclohexane 1:1) yielding 549 mg (93% purity, 77% yield) of the title compound.

LC-MS (Method 10): Rₜ=1.87 min; MS (ESIpos): m/z=315 [M+H]⁺

¹H-NMR (600 MHz, DMSO-d6) δ [ppm]: 8.42 (s, 1H), 7.36-7.31 (m, 2H), 7.19-7.13 (m, 2H), 6.72-6.66 (m, 1H), 5.69 (s, 1H), 4.58-4.48 (m, 2H), 3.99-3.92 (m, 2H), 3.84-3.77 (m, 2H), 1.44 (s, 9H)

### Intermediate 505

### tert-butyl 2-anilino-3-bromo-6,7-dihydropyrazolo[1,5-a]pyrazine-5(4H)-carboxylate

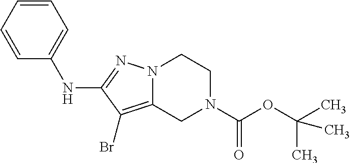 [SMILES: CC(C)(C)OC(=O)N1CCn2nc(Nc3ccccc3)c(Br)c2C1]

To a solution of tert-butyl 2-anilino-6,7-dihydropyrazolo[1,5-a]pyrazine-5(4H)-carboxylate (549 mg, 1.75 mmol, Intermediate 29) in 5 ml DCM, 1-bromopyrrolidine-2,5-dione (311 mg, 1.75 mmol) was added at 0° C. in one portion. The ice bath was removed and the stirred mixture was allowed to warm to rt. Stirring at rt was continued for 18 h. 1-bromopyrrolidine-2,5-dione (100 mg, 0.56 mmol) and stirring was continued for 1 h. NaHCO₃(saturated, aqueous, 1 ml) and DCM (5 ml) were added. The mixture was dried with an water-repellent filter and concentrated under reduced pressure. The residue was purified by silica gel column chromatography (eluent: ethyl acetate/cyclohexane 1:2) yielding 93.0 mg (100% purity, 14% yield) of the title compound.

LC-MS (Method 10): Rₜ=2.19 min; MS (ESIpos): m/z=393 [M+H]⁺1H-NMR (600 MHz, DMSO-d6) δ [ppm]: 7.90 (s, 1H), 7.38-7.34 (m, 2H), 7.20-7.14 (m, 2H), 6.77-6.72 (m, 1H), 4.48-4.43 (m, 2H), 4.03-3.98 (m, 2H), 3.85-3.81 (m, 2H), 1.45 (s, 9H) Intermediate 506

### tert-butyl 2-anilino-3-(pyridin-4-yl)-6,7-dihydropyrazolo[1,5-a]pyrazine-5(4H)-carboxylate

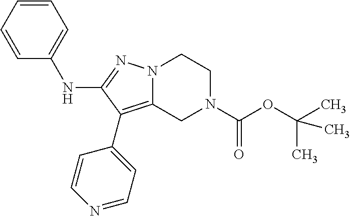 [SMILES: CC(C)(C)OC(=O)N1CCn2nc(Nc3ccccc3)c(-c3ccncc3)c2C1]

In a microwave reaction vessel, tert-butyl 2-anilino-3-bromo-6,7-dihydropyrazolo[1,5-a]pyrazine-5(4H)-carboxylate (93.0 mg, 236 μmol, Intermediate 30) und pyridin-4-ylboronic acid (32.0 mg, 260 μmol) were dissolved in 1,4-dioxane (2 ml) and were flushed with Argon. With stirring, PdCl2(PCy3)2 (14.0 mg, 19.0 μmol; CAS-RN: [29934-17-6]) and aqueous sodium carbonate solution (570 μl, 2.0 M, 1.1 mmol) were added, the vial was sealed and the mixture was heated at 150° C. for 20 min in a microwave oven. After cooling to rt, ethyl acetate and water were added and the mixture was filtered through a pad of celite. The layers were separated and the aqueous layer was extracted with ethyl acetate. The combined organic layers were dried over sodium sulfate, filtered and the solvent was evaporated under reduced pressure. The residue was purified by reverse phase preparative HPLC (Method 5) yielding 62.0 mg (99% purity, 66% yield) of the desired product.

LC-MS (Method 9): Rₜ=1.40 min; MS (ESIpos): m/z=392 [M+H]⁺

¹H-NMR (600 MHz, DMSO-d6) δ [ppm]: 1.418 (16.00), 2.515 (1.32), 2.518 (1.21), 2.521 (0.99), 3.163 (1.24), 3.172 (1.27), 3.866 (1.90), 3.875 (3.38), 3.884 (2.23), 4.061 (2.82), 4.071 (4.36), 4.080 (2.42), 4.088 (0.48), 4.706 (4.56), 6.680 (1.29), 6.691 (2.52), 6.703 (1.42), 7.098 (2.04), 7.112 (5.21), 7.124 (5.30), 7.133 (5.68), 7.146 (2.20), 7.345 (3.32), 7.353 (3.40), 7.987 (2.99), 8.532 (1.91).

### Intermediate 507

### N-phenyl-3-(pyridin-4-yl)-4,5,6,7-tetrahydropyrazolo[1,5-a]pyrazin-2-amine hydrogen chloride (1/2)

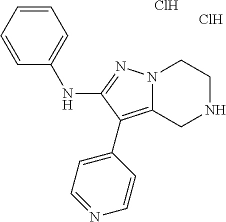 [SMILES: Cl ; c1ccc(Nc2nn3c(c2-c2ccncc2)CNCC3)cc1 ; Cl]

To a solution of tert-butyl 2-anilino-3-(pyridin-4-yl)-6,7-dihydropyrazolo[1,5-a]pyrazine-5(4H)-carboxylate (60.0 mg, 153 μmol, Intermediate 30) in DCM (2 ml) (30 μl, 390 μmol) TFA was added. The mixture was stirred at rt for 18 h. An additional amount of (30 μl, 390 μmol), hydrogen chloride (380 μl, 4.0 M in 1,4.dioxane, 1.5 mmol) was added and stirring at rt was continued for 18 h. The solvent was evaporated and the residue was dried in vacuo to give 62.0 mg (84% purity, 93% yield) of the desired product that was used without further purification.

LC-MS (Method 10): Rₜ=0.34 min; MS (ESIpos): m/z=292 [M+H]⁺

¹H-NMR (600 MHz, DMSO-d6) δ [ppm]: 10.34 (s br, 2H), 8.84-8.78 (m, 2H), 8.52-8.46 (m, 1H), 7.97-7.90 (m, 2H), 7.28-7.15 (m, 4H), 6.83-6.77 (m, 1H), 4.63 (s, 2H), 4.40-4.32 (m, 2H), 3.73-3.65 (m, 3H)

### Intermediate 508

### ethyl 3-bromo-1-{1-[(tert-butoxycarbonyl)amino]propan-2-yl}-1H-pyrazole-5-carboxylate

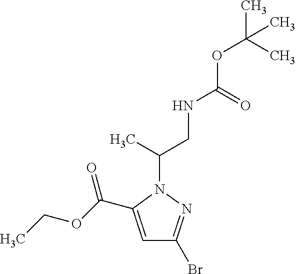 [SMILES: CCOC(=O)c1cc(Br)nn1C(C)CNC(=O)OC(C)(C)C]

tert-butyl(2-hydroxypropyl) carbamate (1.00 g, 5.71 mmol), ethyl 3-bromo-1H-pyrazole-5-carboxylate (1.38 g, 6.28 mmol) and triphenylphosphane (2.25 g, 8.56 mmol; CAS-RN: [603-35-0]) was added to a reaction vessel and THF (15 ml) was added. The vessel was sealed under nitrogen and cooled to 0° C., then dipropan-2-yl (E)-diazene-1,2-dicarboxylate (1.7 ml, 8.6 mmol; CAS-RN: [2446-83-5]) was added and the mixture was warmed to RT and stirred at RT for 2 h. The mixture was concentrated under reduced pressure, then isolute was added and the solvent was removed under reduced pressure. Purification by silica gel column chromatography, (Biotage Sfär Silica HC 25 g column) Eluent: Hexan/EE 5% 2 CV, 5-25% 13 CV Flowrate: 80 mL/min, yielding the title compound (1.17 g, 55% yield) LC-MS (Method 3): Rₜ=1.38 min; MS (ESIpos): m/z=377 [M+H]⁺

¹H-NMR (400 MHz, DMSO-d6) δ [ppm]: 1.276 (1.83), 1.293 (3.59), 1.317 (16.00), 1.355 (3.75), 1.372 (3.77), 1.402 (0.54), 2.518 (1.13), 2.523 (0.80), 3.323 (0.46), 3.355 (0.75), 4.251 (0.61), 4.269 (1.99), 4.287 (2.00), 4.304 (0.64), 6.888 (0.46), 6.932 (1.90).

### Intermediate 509

### tert-butyl {2-[3-bromo-5-(hydroxymethyl)-1H-pyrazol-1-yl]propyl}carbamate

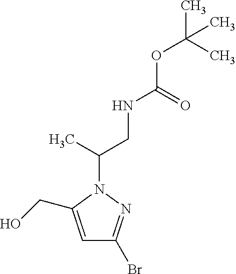 [SMILES: CC(CNC(=O)OC(C)(C)C)n1nc(Br)cc1CO]

Racemic ethyl 3-bromo-1-{1-[(tert-butoxycarbonyl)amino]propan-2-yl}-1H-pyrazole-5-carboxylate (1.17 g, 3.11 mmol, Intermediate 508) was dissolved in ethanol (15 ml) and cooled with an ice bath. Sodium tetrahydroborate (707 mg, 18.7 mmol; CAS-RN: [16940-66-2]) was added and the mixture allowed to warm to RT and stirred over night. Water (20 mL) was cautiously added, and the mixture extracted twice with EtOAc (50 mL) and the organic phase washed with sat. NaCl-solution (20 mL). The organic layer was dried with Na₂SO₄ and concentrated under reduced pressure yielding the crude title compound (1.13 g, 108% yield) which was used in the subsequent step without further purification.

LC-MS (Method 3): Rₜ=1.01 min; MS (ESIpos): m/z=336 [M+H]⁺

### Intermediate 510

### (3-bromo-1-{1-[(tert-butoxycarbonyl)amino]propan-2-yl}-1H-pyrazol-5-yl)methyl methanesulfonate

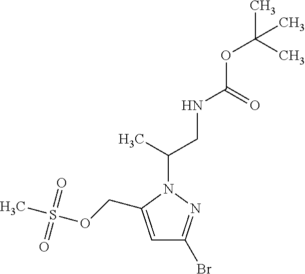 [SMILES: CC(CNC(=O)OC(C)(C)C)n1nc(Br)cc1COS(C)(=O)=O]

Racemic tert-butyl {2-[3-bromo-5-(hydroxymethyl)-1H-pyrazol-1-yl]propyl}carbamate (1.13 g, 3.38 mmol, Intermediate 509) and triethylamine (1.0 ml, 7.4 mmol; CAS-RN: [121-44-8]) were added to THF (10 ml) and the mixture cooled to 0° C. Under nitrogen methanesulfonylchloride (290 μl, 3.7 mmol; CAS-RN: [124-63-0]) was added, and the mixture was stirred for 4 h. An additional quantity of methanesulfonylchloride (1.7 mmol) was added and the mixture stirred for an additional 72 hours at RT. The mixture was poured into NaHCO₃-solution (50% saturated), and extracted twice with EtOAc (50 mL), the organic phase was washed with sat. NaCl-solution (50 mL) and dried over sodium sulfate, and concentrated under reduced pressure yielding the crude title compound (1.18 g, 84% yield) which was used in the next step without further purification.

LC-MS (Method 3): Rₜ=1.12 min; MS (ESIpos): m/z=412 [M+H]⁺

### Intermediate 511

### tert-butyl (7RS)-2-bromo-7-methyl-6,7-dihydropyrazolo[1,5-a]pyrazine-5(4H)-carboxylate

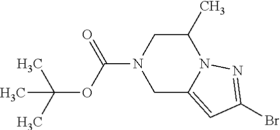 [SMILES: CC1CN(C(=O)OC(C)(C)C)Cc2cc(Br)nn21]

Racemic (3-bromo-1-{1-[(tert-butoxycarbonyl)amino]propan-2-yl}-1H-pyrazol-5-yl)methyl methanesulfonate (1.18 g, 2.85 mmol, Intermediate 510) was dissolved in DMF (10 ml). Under nitrogen, the solution was cooled to 0° C. and sodium hydride (228 mg, 60% purity in mineral oil, 5.70 mmol; CAS-RN: [7646-69-7]) was slowly added and the mixture was stirred overnight at RT. Ice was cautiously added, after it melted the solution was further diluted with water, and the mixture extracted twice with MTBE (50 mL) and the organic phase dried over sodium sulfate, and finally concentrated under reduced pressure yielding the crude title compound (1.05 g, 117% yield, contains mineral oil)

LC-MS (Method 3): Rₜ=1.26 min; MS (ESIpos): m/z=316 [M+H]⁺

### Intermediate 512

### tert-butyl (7RS)-2-bromo-3-iodo-7-methyl-6,7-dihydropyrazolo[1,5-a]pyrazine-5(4H)-carboxylate

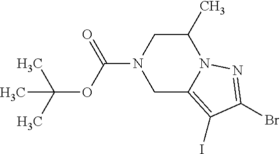 [SMILES: CC1CN(C(=O)OC(C)(C)C)Cc2c(I)c(Br)nn21]

Racemic tert-butyl (7RS)-2-bromo-7-methyl-6,7-dihydropyrazolo[1,5-a]pyrazine-5(4H)-carboxylate (1.05 g, 3.33 mmol, Intermediate 511) and N-lodsuccinimide (750 mg, 3.33 mmol; CAS-RN: [516-12-1]) were dissolved in acetonitrile (10 ml) and stirred at RT over night. The mixture was diluted with water (10 mL), sodium thiosulfate solution (sat. aqueous) was added, and the mixture extracted twice with EtOAc (50 mL), dried over sodium sulfate, and concentrated under reduced pressure yielding the crude title compound as a racemate (1.53 g, 104% yield)

LC-MS (Method 3): Rₜ=1.42 min; MS (ESIpos): m/z=442 [M+H]⁺

### Intermediate 513

### tert-butyl (7RS)-2-bromo-7-methyl-3-(pyridin-4-yl)-6,7-dihydropyrazolo[1,5-a]pyrazine-5(4H)-carboxylate

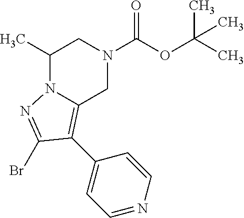 [SMILES: CC1CN(C(=O)OC(C)(C)C)Cc2c(-c3ccncc3)c(Br)nn21]

Racemic tert-butyl (7RS)-2-bromo-3-iodo-7-methyl-6,7-dihydropyrazolo[1,5-a]pyrazine-5(4H)-carboxylate (1.53 g, 3.46 mmol, Intermediate 512), 4-(4,4,5,5-tetramethyl-1,3,2-dioxaborolan-2-yl)pyridine (781 mg, 3.81 mmol), THF (10 ml), potassium carbonate (1.43 g. 10.4 mmol; CAS-RN: [584-08-7]) and water (3.0 ml) were added to a microwave vessel. After degassing the mixture PdCl₂·DPPF·DCM (141 mg, 173 μmol; CAS-RN: [95464-05-4]) was added and the vessel was sealed under nitrogen. The mixture was stirred at 85° C. for 2 h in a microwave. EtOAc (50 ml) was added, and the mixture then washed with sat. NaHCO₃ solution and then sat. NaCl solution (each 35 mL), the organic phase dried over Na₂SO₄, and concentrated under reduced pressure, yielding the racemic crude title material (1.43 g, 105% yield).

LC-MS (Method 3): Rₜ=0.94 min; MS (ESIpos): m/z=394 [M+H]⁺

### Intermediate 514

### tert-butyl-2-bromo-7-methyl-3-(pyridin-4-yl)-6,7-dihydropyrazolo[1,5-a]pyrazine-5(4H)-carboxylate (isomer 1)

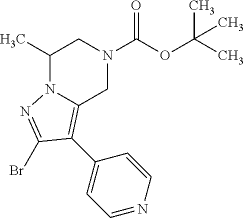 [SMILES: CC1CN(C(=O)OC(C)(C)C)Cc2c(-c3ccncc3)c(Br)nn21]

Racemic crude tert-butyl (7RS)-2-bromo-7-methyl-3-(pyridin-4-yl)-6,7-dihydropyrazolo[1,5-a]pyrazine-5(4H)-carboxylate (1.53 g, 3.89 mmol, Intermediate 513), was purified by reverse phase preparative HPLC chromatography (XBridge C18 5 μm 100×30 mm column), using a gradient of water (containing 1% NH₃ solution) and acetonitrile, with the product containing fractions being identified by LCMS. The fractions were pooled, concentrated under reduced pressure (yielding 423 mg, of the racemic compound), which was duely dissolved in MeCN (25 ml), and filtered. The two enantiomers were separated via chiral SFC chromatography, Chiralpak OZ—H 250×25 column, solvent system CO2 70%/EtOH 30%, with injections of 0.1 ml (~1.6 mg), with a flow rate of 120 ml/min, total run time of 5.6 min, detection at 210 nm, Backpressure of 80 bar and oventemp: 34° C., yielding isomer 1 (123 mg) and isomer 2 (157 mg).

Analytical chiral HPLC: Column: Daicel Chiralpak OZ-3 3 μm, 100×4.6 mm, Temp.: 40° C.,

Eluent: 70% CO2: 30% EtOH, Flow rate: 3.0 mL/min, Run time: 10 min, UV: 210 nm, Inj.: 5 μl

SFC-Pressure: 130 bar, SFC—BPR Temp.: 60° C. Rf Isomer 1=1.957 minutes.

### Intermediate 515

### tert-butyl-2-bromo-7-methyl-3-(pyridin-4-yl)-6,7-dihydropyrazolo[1,5-a]pyrazine-5(4H)-carboxylate (isomer 2)

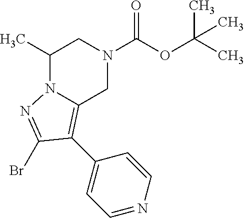 [SMILES: CC1CN(C(=O)OC(C)(C)C)Cc2c(-c3ccncc3)c(Br)nn21]

Second enantiomer from separation of tert-butyl (7RS)-2-bromo-7-methyl-3-(pyridin-4-yl)-6,7-dihydropyrazolo[1,5-a]pyrazine-5(4H)-carboxylate (Intermediate 513).

Analytical chiral HPLC: Column: Daicel Chiralpak OZ-3 3 μm, 100×4.6 mm, Temp.: 40° C., Eluent: 70% CO2: 30% EtOH, Flow rate: 3.0 mL/min, Run time: 10 min, UV: 210 nm, Inj.: 5 μl

SFC-Pressure: 130 bar, SFC—BPR Temp.: 60° C. Rf Isomer 2=2.501 minutes.

### Intermediate 516

### tert-butyl)-2-(4-methoxyphenyl)-7-methyl-3-(pyridin-4-yl)-6,7-dihydropyrazolo[1,5-a]pyrazine-5(4H)-carboxylate (isomer 2)

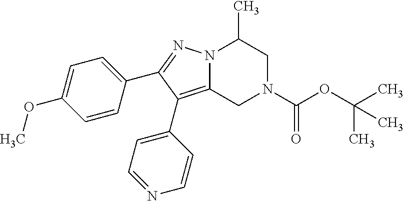 [SMILES: COc1ccc(-c2nn3c(c2-c2ccncc2)CN(C(=O)OC(C)(C)C)CC3C)cc1]

Under argon, tert-butyl-2-bromo-7-methyl-3-(pyridin-4-yl)-6,7-dihydropyrazolo[1,5-a]pyrazine-5(4H)-carboxylate (isomer 2) (157 mg, 399 μmol, Intermediate 515) was dissolved in dioxane (4 ml), (4-methoxyphenyl) boronic acid (72.8 mg, 479 μmol) and water (0.4 ml) were added and the mixture was degassed with argon for 15 min. Sodiumcarbonate (84.6 mg, 798 μmol; CAS-RN: [497-19-8]) and PdCl2(dppf) (32.6 mg, 39.9 μmol; CAS-RN: [95464-05-4]) were added, and the mixture degassed further for 5 min, sealed and stirred at 80° C. over night. The mixture was diluted with water, extracted twice into EtoAc, dried using a hydrophobic filter, evaporated under reduced pressure and purified via RP-HPLC yielding the title compound (117 mg (99% purity, 69% yield)

LC-MS (Method 1): Rₜ=0.93 min; MS (ESIpos): m/z=421 [M+H]⁺

¹H-NMR (400 MHz, DMSO-d6) δ [ppm]: 1.412 (7.28), 1.458 (4.43), 1.475 (4.37), 2.518 (0.95), 2.523 (0.60), 3.752 (16.00), 3.907 (0.64), 3.917 (0.77), 3.941 (0.59), 3.951 (0.52), 4.399 (0.55), 4.410 (0.54), 4.634 (0.51), 4.692 (1.67), 4.734 (0.75), 6.901 (3.15), 6.906 (0.95), 6.918 (1.11), 6.923 (3.47), 7.128 (3.13), 7.131 (1.87), 7.138 (1.87), 7.143 (3.10), 7.252 (2.19), 7.275 (1.85), 8.511 (2.99), 8.515 (1.74), 8.523 (1.78), 8.527 (2.78).

### Intermediate 517

### tert-butyl)-2-(4-methoxyphenyl)-7-methyl-3-(pyridin-4-yl)-6,7-dihydropyrazolo[1,5-a]pyrazine-5(4H)-carboxylate (isomer 1)

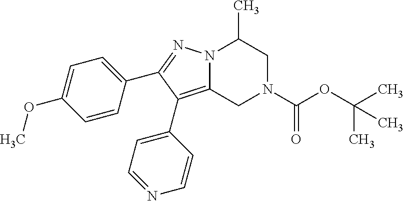 [SMILES: COc1ccc(-c2nn3c(c2-c2ccncc2)CN(C(=O)OC(C)(C)C)CC3C)cc1]

Under argon, tert-butyl-2-bromo-7-methyl-3-(pyridin-4-yl)-6,7-dihydropyrazolo[1,5-a]pyrazine-5(4H)-carboxylate (isomer 1) (123 mg, 399 μmol, Intermediate 514) produced analogously to the protocol used to produce Intermediate 516, yielding the title compound (86 mg (99% purity, 65% yield)

LC-MS (Method 1): Rₜ=0.92 min; MS (ESIpos): m/z=421 [M+H]⁺

¹H-NMR (400 MHz, DMSO-d6) δ [ppm]: 1.412 (6.82), 1.458 (4.23), 1.475 (4.17), 2.518 (0.96), 2.522 (0.62), 3.752 (16.00), 3.906 (0.60), 3.917 (0.73), 3.941 (0.56), 3.951 (0.50), 4.399 (0.52), 4.410 (0.51), 4.634 (0.47), 4.692 (1.58), 4.734 (0.71), 6.901 (3.07), 6.906 (0.94), 6.918 (1.06), 6.923 (3.47), 7.127 (3.05), 7.131 (1.87), 7.138 (1.87), 7.143 (3.02), 7.252 (2.08), 7.274 (1.77), 8.511 (3.10), 8.515 (1.78), 8.523 (1.71), 8.527 (2.90).

### Intermediate 518

### 2-(4-methoxyphenyl)-7-methyl-3-(pyridin-4-yl)-4,5,6,7-tetrahydropyrazolo[1,5-a]pyrazine-hydrogen chloride (1/1) (isomer 2)

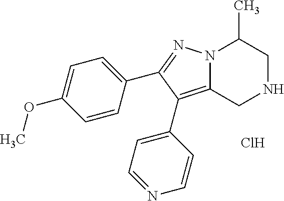 [SMILES: COc1ccc(-c2nn3c(c2-c2ccncc2)CNCC3C)cc1 ; Cl]

Under argon, tert-butyl-2-(4-methoxyphenyl)-7-methyl-3-(pyridin-4-yl)-6,7-dihydropyrazolo[1,5-a]pyrazine-5(4H)-carboxylate (116 mg, 276 μmol, Intermediate 516) was dissolved in DCM (3.0 ml), hydrochloric acid (1.0 ml, 4.0 M, 4.1 mmol in dioxane; CAS-RN: [7647-01-0]) was added and the reaction mixture was stirred at RT for 2 h. The mixture was evaporated in vacuum and used in the next step without further purification.

LC-MS (Method 3): Rₜ=0.91 min; MS (ESIpos): m/z=321 [M+H]⁺

### Intermediate 519

### 2-(4-methoxyphenyl)-7-methyl-3-(pyridin-4-yl)-4,5,6,7-tetrahydropyrazolo[1,5-a]pyrazine-hydrogen chloride (1/1) (isomer 1)

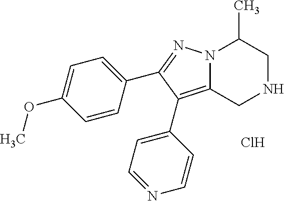 [SMILES: COc1ccc(-c2nn3c(c2-c2ccncc2)CNCC3C)cc1 ; Cl]

Under argon, tert-butyl-2-(4-methoxyphenyl)-7-methyl-3-(pyridin-4-yl)-6,7-dihydropyrazolo[1,5-a]pyrazine-5(4H)-carboxylate (isomer 1) (85.0 mg, 202 μmol, Intermediate 517) was dissolved in DCM (2.2 ml), hydrochloric acid (760 μl, 4.0 M in dioxane, 3.0 mmol; CAS-RN: [7647-01-0]) was added and the reaction mixture was stirred at RT for 2 h. The mixture was evaporated in vacuum and used in the next step without further purification.

LC-MS (Method 3): Rₜ=0.91 min; MS (ESIpos): m/z=321 [M+H]⁺

### Intermediate 520

### methyl 3-bromo-1-{(2R)-2-[(tert-butoxycarbonyl)amino]propyl}-1H-pyrazole-5-carboxylate

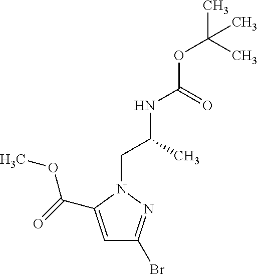 [SMILES: COC(=O)c1cc(Br)nn1C[C@@H](C)NC(=O)OC(C)(C)C]

To a solution of methyl 3-bromo-1H-pyrazole-5-carboxylate (5.00 g, 24.4 mmol) and tert-butyl [(2R)-1-hydroxypropan-2-yl]carbamate (5.13 g, 29.3 mmol) in tetrahydrofuran (200 ml) were added triphenylphosphine (12.8 g, 48.8 mmol) and diisopropyl azodicarboxylate (9.86 g, 48.8 mmol) at 20° C. The mixture was stirred at 20° C. for 16 hours. The reaction mixture was poured into water and extracted with ethyl acetate. The combined organic phase was concentrated in vacuo to give a crude product. The crude product was purified by flash column on silica gel (petroleum ether:ethyl acetate=100:1 to 4:1) to give methyl 3-bromo-1-{(2R)-2-[(tert-butoxycarbonyl)amino]propyl}-1H-pyrazole-5-carboxylate (8.80 g, 24.3 mmol, 99% yield) as a yellow oil.

LC-MS (Method 11): Rₜ=0.555 min; MS (ESIpos): m/z=305.9 [M−56]⁺.

### Intermediate 521

### 3-bromo-1-{(2R)-2-[(tert-butoxycarbonyl)amino]propyl}-1H-pyrazole-5-carboxylic acid

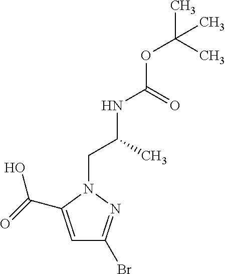 [SMILES: C[C@H](Cn1nc(Br)cc1C(=O)O)NC(=O)OC(C)(C)C]

To a solution of methyl 3-bromo-1-{(2R)-2-[(tert-butoxycarbonyl)amino]propyl}-1H-pyrazole-5-carboxylate (8.80 g, 24.3 mmol, Intermediate 508) in ethanol (50 ml) was added a solution of potassium hydroxide (6.82 g, 121 mmol) in water (100 ml) at 20° C. The mixture was stirred at 20° C. for 16 hours. pH of the reaction mixture was adjusted to 4~6 with hydrochloric acid (1M in water). The resulting suspension was filtered and the solid cake was collected to give 3-bromo-1-{(2R)-2-[(tert-butoxycarbonyl)amino]propyl}-1H-pyrazole-5-carboxylic acid (8.10 g, 23.3 mmol, 96% yield) as an off-white solid.

LC-MS (Method 12): Rₜ=0.503 min; MS (ESIpos): m/z=247.9 [M−100]⁺.

### Intermediate 522

### tert-butyl [(2R)-1-{3-bromo-5-[methoxy(methyl) carbamoyl]-1H-pyrazol-1-yl}propan-2-yl]carbamate

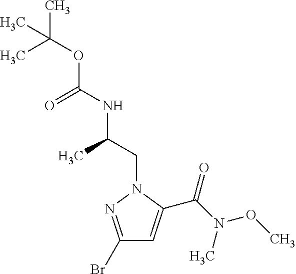 [SMILES: CON(C)C(=O)c1cc(Br)nn1C[C@@H](C)NC(=O)OC(C)(C)C]

To a solution of 3-bromo-1-{(2R)-2-[(tert-butoxycarbonyl)amino]propyl}-1H-pyrazole-5-carboxylic acid (8.10 g, 23.3 mmol, Intermediate 521) and N,O-dimethylhydroxylamine hydrochloride (1:1) (3.40 g, 34.9 mmol) in N,N-dimethylformamide (120 ml) were added O-(7-azabenzotriazol-1-yl)-N,N,N,N-tetramethyl uronium hexafluorophosphate (13.3 g, 34.9 mmol) and N,N-diisopropylethylamine (9.02 g, 69.8 mmol) at 20° C. The mixture was stirred at 20° C. for 16 hours. The reaction mixture was poured into water and extracted with ethyl acetate. The combined organic phase was washed with brine, dried over anhydrous sodium sulfate, filtered and concentrated in vacuo to give a crude product. The crude product was purified by column chromatography on silica gel (petroleum ether:ethyl acetate=50:1 to 3:2) to give tert-butyl [(2R)-1-{3-bromo-5-[methoxy(methyl) carbamoyl]-1H-pyrazol-1-yl}propan-2-yl]carbamate (7.80 g, 19.9 mmol, 86% yield) as a white solid.

LC-MS (Method 11): Rₜ=0.809 min; MS (ESIpos): m/z=393.0 [M+H]⁺.

### Intermediate 523

### tert-butyl [(2R)-1-(5-acetyl-3-bromo-1H-pyrazol-1-yl) propan-2-yl]carbamate

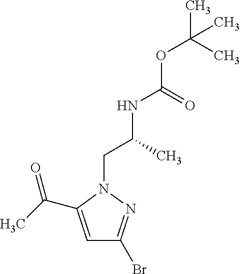 [SMILES: CC(=O)c1cc(Br)nn1C[C@@H](C)NC(=O)OC(C)(C)C]

To a solution of tert-butyl [(2R)-1-{3-bromo-5-[methoxy(methyl) carbamoyl]-1H-pyrazol-1-yl}propan-2-yl]carbamate (7.80 g, 19.9 mmol, Intermediate 522) in tetrahydrofuran (150 ml) was slowly added bromide(methyl) magnesium (10 ml, 30.0 mmol, 3.0 M in tetrahydrofuran) at −78° C. under nitrogen atmosphere. After the completion of addition, the reaction mixture was allowed to stir at 20° C. for 4 hours. The reaction was quenched with saturated ammonium chloride aqueous solution and the resulting mixture was extracted with ethyl acetate. The combined organic phase was washed with birne, dried over anhydrous sodium sulfate, filtered and concentrated in vacuo to give a crude product. The crude product was purified by column chromatography on silica gel (petroleum ether:ethyl acetate=100:1 to 4:1) to give tert-butyl [(2R)-1-(5-acetyl-3-bromo-1H-pyrazol-1-yl) propan-2-yl]carbamate (4.80 g, 13.9 mmol, 70% yield) as an off-white solid.

LC-MS (Method 11): Rₜ=0.903 min; MS (ESIpos): m/z=346.1 [M+H]⁺.

### Intermediate 524

### (6R)-2-bromo-4,6-dimethyl-6,7-dihydropyrazolo[1,5-a]pyrazine

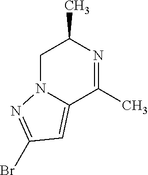

To a solution of tert-butyl [(2R)-1-(5-acetyl-3-bromo-1H-pyrazol-1-yl) propan-2-yl]carbamate (4.80 g, 13.9 mmol, Intermediate 523) in dichloromethane (60 ml) was added trifluoroacetic acid (29.6 g, 260 mmol) at 20° C. and the mixture was stirred at 20° C. for 16 hours. The reaction mixture was concentrated in vacuo to give (6R)-2-bromo-4,6-dimethyl-6,7-dihydropyrazolo[1,5-a]pyrazine (3.10 g, 13.6 mmol, 98% yield) as a brown solid.

LC-MS (Method 11): Rₜ=0.279 min; MS (ESIpos): m/z=230.0 [M+H]⁺.

### Intermediate 525

### (4RS,6R)-2-bromo-4,6-dimethyl-4,5,6,7-tetrahydropyrazolo[1,5-a]pyrazine

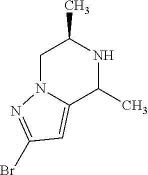

To a solution of (6R)-2-bromo-4,6-dimethyl-6,7-dihydropyrazolo[1,5-a]pyrazine (3.10 g, 13.6 mmol, Intermediate 524) in methanol (50 ml) were added sodium cyanoborohydride (1.71 g, 27.2 mmol) and acetic acid (0.816 g, 13.6 mmol) at 0° C. The mixture was stirred at 20° C. for 2 hours. The reaction mixture was concentrated in vacuo to give (4RS,6R)-2-bromo-4,6-dimethyl-4,5,6,7-tetrahydropyrazolo[1,5-a]pyrazine (3.10 g, 13.5 mmol, 99% yield) as an off-white solid.

LC-MS (Method 11): Rₜ=0.172 min; MS (ESIpos): m/z=232.1 [M+H]⁺.

### Intermediate 526

### tert-butyl (4RS,6R)-2-bromo-4,6-dimethyl-6,7-dihydropyrazolo[1,5-a]pyrazine-5(4H)-carboxylate

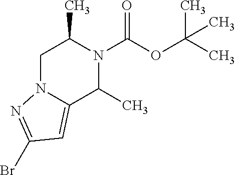 [SMILES: CC1c2cc(Br)nn2C[C@@H](C)N1C(=O)OC(C)(C)C]

To a solution of (4RS,6R)-2-bromo-4,6-dimethyl-4,5,6,7-tetrahydropyrazolo[1,5-a]pyrazine (3.10 g, 13.5 mmol, Intermediate 525) in dichloromethane (60 ml) were added triethylamine (4.09 g, 40.4 mmol) and di-tert-butyl carbonate (4.69 g, 26.9 mmol) at 20° C. The mixture was stirred at 20° C. for 16 hours. The reaction mixture was poured into water and extracted with ethyl acetate. The combined organic phase was concentrated in vacuo to give a crude product, which was purified by column chromatography on silica gel (petroleum ether:ethyl acetate=100:1 to 5:1) to give tert-butyl (4RS,6R)-2-bromo-4,6-dimethyl-6,7-dihydropyrazolo[1,5-a]pyrazine-5(4H)-carboxylate (4.20 g, 12.7 mmol, 94% yield) as a colorless oil.

LC-MS (Method 11): Rₜ=1.192 min; MS (ESIpos): m/z=332.1 [M+H]⁺.

### Intermediate 527

### tert-butyl (4RS,6R)-2-(4-chloro-2-fluorophenyl)-4,6-dimethyl-6,7-dihydropyrazolo[1,5-a]pyrazine-5(4H)-carboxylate

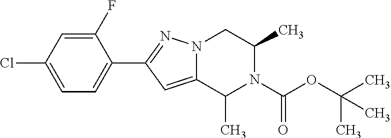 [SMILES: CC1c2cc(-c3ccc(Cl)cc3F)nn2C[C@@H](C)N1C(=O)OC(C)(C)C]

To a solution of tert-butyl (4RS,6R)-2-bromo-4,6-dimethyl-6,7-dihydropyrazolo[1,5-a]pyrazine-5(4H)-carboxylate (1.00 g, 3.03 mmol, Intermediate 526) and (4-chloro-2-fluorophenyl) boronic acid (0.581 g, 3.33 mmol) in 1,4-dioxane (30 ml) and water (10 ml) were added sodium carbonate (0.481 g, 4.54 mmol) and (1,1′-bis(diphenylphosphino) ferrocene) palladium (II) dichloride (0.222 g, 0.303 mmol) at 80° C. The mixture was stirred at 80° C. for 16 hours under nitrogen atmosphere. The reaction mixture was poured into water and extracted with ethyl acetate. The combined organic phase was concentrated to give a crude product, which was purified by column chromatography on silica gel (petroleum ether:ethyl acetate=100:1 to 1:1) to give tert-butyl (4RS,6R)-2-(4-chloro-2-fluorophenyl)-4,6-dimethyl-6,7-dihydropyrazolo[1,5-a]pyrazine-5(4H)-carboxylate (1.00 g, 2.63 mmol, 87% yield) as a yellow oil.

LC-MS (Method 11): Rₜ=0.724 min; MS (ESIpos): m/z=380.1 [M+H]⁺.

### Intermediate 528

### tert-butyl (4RS,6R)-2-(4-chloro-2-fluorophenyl)-3-iodo-4,6-dimethyl-6,7-dihydropyrazolo[1,5-a]pyrazine-5(4H)-carboxylate

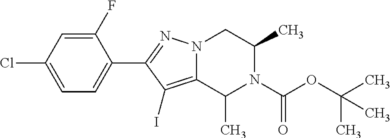 [SMILES: CC1c2c(I)c(-c3ccc(Cl)cc3F)nn2C[C@@H](C)N1C(=O)OC(C)(C)C]

To a solution of tert-butyl (4RS,6R)-2-(4-chloro-2-fluorophenyl)-4,6-dimethyl-6,7-dihydropyrazolo[1,5-a]pyrazine-5(4H)-carboxylate (1.00 g, 2.63 mmol, Intermediate 527) in N,N-dimethylformamide (30 ml) was added 1-iodopyrrolidine-2,5-dione (1.18 g, 5.27 mmol) at 20° C. The mixture was stirred at 45° C. for 16 hours. The reaction mixture was poured into water and extracted with ethyl acetate. The combined organic phase was concentrated in vacuo to give a crude product, which was purified by column chromatography on silica gel (petreoleum ether:ethyl acetate=100:1 to 3:1) to give tert-butyl (4RS,6R)-2-(4-chloro-2-fluorophenyl)-3-iodo-4,6-dimethyl-6,7-dihydropyrazolo[1,5-a]pyrazine-5(4H)-carboxylate (1.20 g, 2.37 mmol, 90% yield) as a yellow solid.

LC-MS (Method 11): Rₜ=0.731 min; MS (ESIpos): m/z=506.1 [M+H]⁺.

### Intermediate 529

### tert-butyl (4RS,6R)-2-(4-chloro-2-fluorophenyl)-4,6-dimethyl-3-(pyridin-4-yl)-6,7-dihydropyrazolo[1,5-a]pyrazine-5(4H)-carboxylate

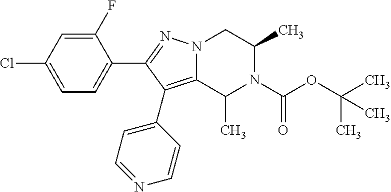 [SMILES: CC1c2c(-c3ccncc3)c(-c3ccc(Cl)cc3F)nn2C[C@@H](C)N1C(=O)OC(C)(C)C]

To a solution of tert-butyl (4RS,6R)-2-(4-chloro-2-fluorophenyl)-3-iodo-4,6-dimethyl-6,7-dihydropyrazolo[1,5-a]pyrazine-5(4H)-carboxylate (1.20 g, 2.37 mmol, Intermediate 528) and pyridin-4-ylboronic acid (0.350 g, 2.85 mmol) in 1,4-dioxane (30 ml) and water (8 ml) were added sodium carbonate (0.503 g, 4.75 mmol) and (1,1′-bis(diphenylphosphino) ferrocene) palladium (II) dichloride (0.174 g, 0.237 mmol) at 20° C. The mixture was stirred at 80° C. for 16 hours under nitrogen atmosphere. The reaction mixture was poured into water and extracted with ethyl acetate. The combined organic phase was concentrated in vacuo to give a crude product, which was purified by column chromatography on silica gel (petroleum ether:ethyl acetate=100:1 to 3:1) to give tert-butyl (4RS,6R)-2-(4-chloro-2-fluorophenyl)-4,6-dimethyl-3-(pyridin-4-yl)-6,7-dihydropyrazolo[1,5-a]pyrazine-5(4H)-carboxylate (0.800 g, 1.75 mmol, 74% yield) as a yellow solid.

LC-MS (Method 11): Rₜ=0.851 min; MS (ESIpos): m/z=457.2 [M+H]⁺.

Intermediate 530 (4RS,6R)-2-(4-chloro-2-fluorophenyl)-4,6-dimethyl-3-(pyridin-4-yl)-4,5,6,7-tetrahydropyrazolo[1,5-a]pyrazine hydrogen chloride (1/1)

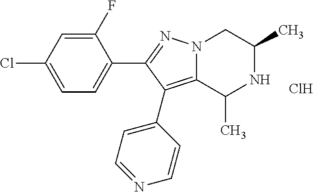 [SMILES: CC1N[C@H](C)Cn2nc(-c3ccc(Cl)cc3F)c(-c3ccncc3)c21 ; Cl]

To a solution of tert-butyl (4RS,6R)-2-(4-chloro-2-fluorophenyl)-4,6-dimethyl-3-(pyridin-4-yl)-6,7-dihydropyrazolo[1,5-a]pyrazine-5(4H)-carboxylate (800 mg, 1.75 mmol, Intermediate 529) in ethyl acetate (30 ml) was added hydrochloric acid (30 ml, 120 mmol, 4 M in ethyl acetate) at 20° C. The mixture was stirred at 20° C. for 1 hour. The reaction mixture was concentrated in vacuo to give (4RS,6R)-2-(4-chloro-2-fluorophenyl)-4,6-dimethyl-3-(pyridin-4-yl)-4,5,6,7-tetrahydropyrazolo[1,5-a]pyrazine hydrogen chloride (1/1) (700 mg, 1.78 mmol, 100% yield) as an off-white solid.

LC-MS (Method 11): R₁=0.464 min; MS (ESIpos): m/z=357.1 [(M−36)+H]⁺.

### Intermediate 531

### 1-[(4RS,6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one

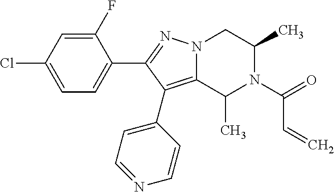 [SMILES: C=CC(=O)N1C(C)c2c(-c3ccncc3)c(-c3ccc(Cl)cc3F)nn2C[C@H]1C]

To a solution of (4RS,6R)-2-(4-chloro-2-fluorophenyl)-4,6-dimethyl-3-(pyridin-4-yl)-4,5,6,7-tetrahydropyrazolo[1,5-a]pyrazine hydrogen chloride (1/1) (400 mg, 1.02 mmol, Intermediate 530) in dichloromethane (50 ml) were added triethylamine (309 mg, 3.05 mmol) and acryloyl chloride (92.1 mg, 1.02 mmol) at −10° C. The mixture was stirred at −10° C. for 1 hour. The reaction mixture was concentrated in vacuo to give a crude product, which was purified by preparative HPLC [Instrument: GX-A; Column: Waters Xbridge 150*25 mm*10 μm; eluent A: water (0.2% FA), eluent B: acetonitrile; gradient: 0-9 min 24-42% B; flow 25 ml/min; temperature: RT; Detector: UV 220/254 nm] to give 1-[(4RS,6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (210 mg, 0.511 mmol, 50% yield) as a white solid.

LC-MS (Method 11): Rₜ=0.459 min; MS (ESIpos): m/z=411.1 [M+H]⁺

### Intermediate 532

### methyl 3-bromo-1-{(2S)-2-[(tert-butoxycarbonyl)amino]propyl}-1H-pyrazole-5-carboxylate

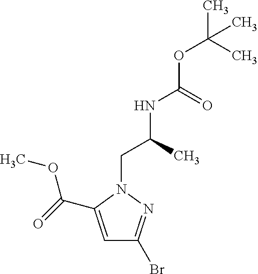 [SMILES: COC(=O)c1cc(Br)nn1C[C@H](C)NC(=O)OC(C)(C)C]

To a solution of methyl 3-bromo-1H-pyrazole-5-carboxylate (5.00 g, 24.4 mmol) and tert-butyl [(2S)-1-hydroxypropan-2-yl]carbamate (5.13 g, 29.3 mmol) in tetrahydrofuran (200 ml) were added triphenylphosphine (12.79 g, 48.8 mmol) and diisopropyl azodicarboxylate (9.86 g, 48.8 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 3:2) to give methyl 3-bromo-1-{(2S)-2-[(tert-butoxycarbonyl)amino]propyl}-1H-pyrazole-5-carboxylate (8.80 g, 24.3 mmol, 100% yield) as a yellow oil.

LC-MS (Method 11): Rₜ=0.766 min; MS (ESIpos): m/z=305.9 [M−56]⁺.

### Intermediate 533

### 3-bromo-1-{(2S)-2-[(tert-butoxycarbonyl)amino]propyl}-1H-pyrazole-5-carboxylic acid

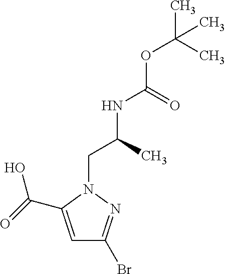 [SMILES: C[C@@H](Cn1nc(Br)cc1C(=O)O)NC(=O)OC(C)(C)C]

To a solution of methyl 3-bromo-1-{(2S)-2-[(tert-butoxycarbonyl)amino]propyl}-1H-pyrazole-5-carboxylate (8.80 g, 24.3 mmol, Intermediate 532) in ethanol (72 ml) was added a solution of potassium hydroxide (6.82 g, 121 mmol) in water (100 ml) at 20° C. The mixture was stirred at 20° C. for 16 hours. The organic solvent was removed under reduced pressure, pH of the residue was adjusted to 4~6 with hydrochloric acid (1 M in water) and the resulting mixture was extracted with ethyl acetate. The combined organic layers were washed with brine, dried over anhydrous sodium sulfate, filtered and concentrated in vacuo to give 3-bromo-1-{(2S)-2-[(tert-butoxycarbonyl)amino]propyl}-1H-pyrazole-5-carboxylic acid (8.40 g, 24.1 mmol, 99% yield) as a white solid.

LC-MS (Method 11): Rₜ=0.486 min; MS (ESIpos): m/z=248.1 [M−100]⁺.

### Intermediate 534

### tert-butyl [(2S)-1-{3-bromo-5-[methoxy(methyl) carbamoyl]-1H-pyrazol-1-yl}propan-2-yl]carbamate

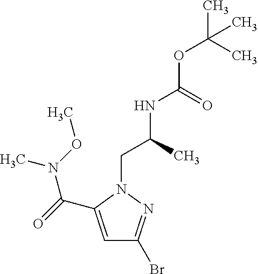 [SMILES: CON(C)C(=O)c1cc(Br)nn1C[C@H](C)NC(=O)OC(C)(C)C]

To a solution of 3-bromo-1-{(2S)-2-[(tert-butoxycarbonyl)amino]propyl}-1H-pyrazole-5-carboxylic acid (8.40 g, 24.1 mmol, Intermediate 533) and N,O-dimethylhydroxylamine hydrochloride (1:1) (3.53 g, 36.2 mmol) in N,N-dimethylformamide (200 ml) were added O-(7-azabenzotriazol-1-yl)-N,N,N,N-tetramethyl uronium hexafluorophosphate (13.8 g, 36.2 mmol) and N,N-diisopropylethylamine (13 ml, 72.0 mmol) at 20° C. The mixture was stirred at 20° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl [(2S)-1-{3-bromo-5-[methoxy(methyl) carbamoyl]-1H-pyrazol-1-yl}propan-2-yl]carbamate (9.40 g, 24.0 mmol, 99% yield) as a yellow oil.

LC-MS (Method 11): Rₜ=0.869 min; MS (ESIpos): m/z=293.0 [M−100]⁺.

### Intermediate 535

### tert-butyl [(2S)-1-(5-acetyl-3-bromo-1H-pyrazol-1-yl) propan-2-yl]carbamate

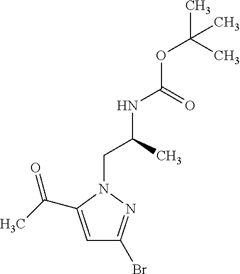 [SMILES: CC(=O)c1cc(Br)nn1C[C@H](C)NC(=O)OC(C)(C)C]

To a solution of tert-butyl [(2S)-1-{3-bromo-5-[methoxy(methyl) carbamoyl]-1H-pyrazol-1-yl}propan-2-yl]carbamate (9.40 g, 24.0 mmol, Intermediate 534) in tetrahydrofuran (100 ml) was slowly added bromido(methyl)magnesium (20 ml, 60.0 mmol, 3.0 M in tetrahydrofuran) at −78° C. under nitrogen atmosphere. After the addition completed, the reaction mixture was allowed to stir at 20° C. for 4 hours. The reaction was quenched with saturated ammonium chloride aqueous solution and the resulting mixture was extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=2:1 to 3:2) to give tert-butyl [(2S)-1-(5-acetyl-3-bromo-1H-pyrazol-1-yl) propan-2-yl]carbamate (6.15 g, 18.5 mmol, 74% yield) as a colorless oil.

LC-MS (Method 11): Rₜ=0.775 min; MS (ESIpos): m/z=246.3 [M−100]⁺.

### Intermediate 536

### (6S)-2-bromo-4,6-dimethyl-6,7-dihydropyrazolo[1,5-a]pyrazine

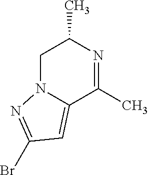

To a solution of tert-butyl [(2S)-1-(3-bromo-5-formyl-1H-pyrazol-1-yl) propan-2-yl]carbamate (6.15 g, 18.5 mmol, Intermediate 535) in dichloromethane (60 ml) was added trifluoroacetic acid (20 ml, 260 mmol) at 20° C. The reaction mixture was stirred at 20° C. for 16 hours. The reaction mixture was concentrated in vacuo to give (6S)-2-bromo-4,6-dimethyl-6,7-dihydropyrazolo[1,5-a]pyrazine (4.20 g, 18.4 mmol, 99% yield) as a yellow oil.

LC-MS (Method 11): Rₜ=0.156 min; MS (ESIpos): m/z=228.0 [M+H]⁺

### Intermediate 537

### (4RS,6S)-2-bromo-4,6-dimethyl-4,5,6,7-tetrahydropyrazolo[1,5-a]pyrazine

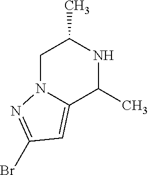

To a solution of (6S)-2-bromo-4,6-dimethyl-6,7-dihydropyrazolo[1,5-a]pyrazine (4.30 g, 18.9 mmol, Intermediate 536) in methanol (100 ml) was added sodium cyanoborohydride (5.92 g, 94.3 mmol) at 20° C. The mixture was stirred at 20° C. for 1 hour. The reaction mixture was concentrated under reduced pressure to give a residue. To the residue was added water, and the resulting mixture was extracted with ethyl acetate. The combined organic layers were washed with brine, dried over anhydrous sodium sulfate, filtered and concentrated in vacuo to give (4RS,6S)-2-bromo-4,6-dimethyl-4,5,6,7-tetrahydropyrazolo[1,5-a]pyrazine (4.30 g, 18.7 mmol, 99% yield) as a yellow oil.

LC-MS (Method 14): Rₜ=0.196 min; MS (ESIpos): m/z=232.1 [M+H]⁺.

### Intermediate 538

### tert-butyl (4RS,6S)-2-bromo-4,6-dimethyl-6,7-dihydropyrazolo[1,5-a]pyrazine-5(4H)-carboxylate

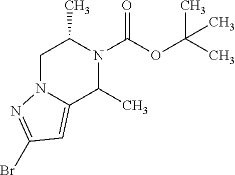 [SMILES: CC1c2cc(Br)nn2C[C@H](C)N1C(=O)OC(C)(C)C]

To a solution of (4RS,6S)-2-bromo-4,6-dimethyl-4,5,6,7-tetrahydropyrazolo[1,5-a]pyrazine (4.30 g, 18.7 mmol, Intermediate 537) in dichloromethane (150 ml) were added triethylamine (7.8 ml, 56.0 mmol) and di-tert-butyl carbonate (8.6 ml, 37.0 mmol) at 20° C.

The mixture was stirred at 20° C. for 16 hours. The reaction mixture was poured into water and extracted with ethyl acetate. The combined organic phase was concentrated in vacuo to give a residue. The residue was purified by column chromatography on silica gel (petroleum ether:ethyl acetate=9:1 to 4:1) to give tert-butyl (4RS,6S)-2-bromo-4,6-dimethyl-6,7-dihydropyrazolo[1,5-a]pyrazine-5(4H)-carboxylate (5.00 g, 18.7 mmol, 81% yield) as a yellow oil.

Optical rotation=+61.271

LC-MS (Method 14): Rₜ=1.149 min; MS (ESIpos): m/z=332.1 [M+H]⁺.

### Intermediate 539

### tert-butyl (4RS,6S)-2-(4-chloro-2-fluorophenyl)-4,6-dimethyl-6,7-dihydropyrazolo[1,5-a]pyrazine-5(4H)-carboxylate

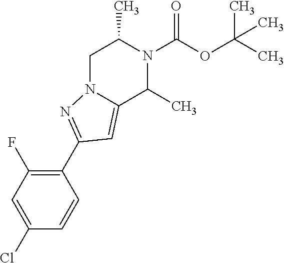 [SMILES: CC1c2cc(-c3ccc(Cl)cc3F)nn2C[C@H](C)N1C(=O)OC(C)(C)C]

To a solution of tert-butyl (4RS,6S)-2-bromo-4,6-dimethyl-6,7-dihydropyrazolo[1,5-a]pyrazine-5(4H)-carboxylate (1.00 g, 3.03 mmol, Intermediate 538) and (4-chloro-2-fluorophenyl) boronic acid (792 mg, 4.54 mmol) in 1,4-dioxane (10 ml) and water (2 ml) were added sodium carbonate (642 mg, 6.05 mmol) and (1,1′-bis(diphenylphosphino)ferrocene)palladium (II) dichloride (221 mg, 0.303 mmol) at 80° C.

The mixture was stirred at 80° C. for 16 hours under nitrogen atmosphere. The reaction mixture was poured into water and extracted with ethyl acetate. The combined organic phase was concentrated in vacuo to give a residue. The residue was purified by column chromatography on silica gel (petroleum ether:ethyl acetate=4:1 to 2:1) to give tert-butyl (4RS,6S)-2-(4-chloro-2-fluorophenyl)-4,6-dimethyl-6,7-dihydropyrazolo[1,5-a]pyrazine-5(4H)-carboxylate (700 mg, 1.84 mmol, 60% yield) as a yellow oil.

LC-MS (Method 11): Rₜ=0.974 min; MS (ESIpos): m/z=380.1 [M+H]⁺.

### Intermediate 540

### tert-butyl (4RS,6S)-2-(4-chloro-2-fluorophenyl)-3-iodo-4,6-dimethyl-6,7-dihydropyrazolo[1,5-a]pyrazine-5(4H)-carboxylate

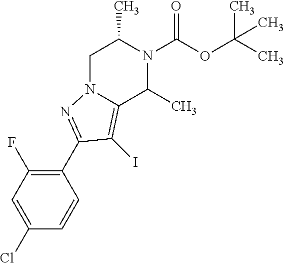 [SMILES: CC1c2c(I)c(-c3ccc(Cl)cc3F)nn2C[C@H](C)N1C(=O)OC(C)(C)C]

To a solution of tert-butyl (4RS,6S)-2-(4-chloro-2-fluorophenyl)-4,6-dimethyl-6,7-dihydropyrazolo[1,5-a]pyrazine-5(4H)-carboxylate (700 mg, 1.84 mmol, Intermediate 539) in dichloromethane (10 ml) and methanol (5 ml) was added 1-iodopyrrolidine-2,5-dione (829 mg, 3.69 mmol) at 20° C. The mixture was stirred at 45° C. for 16 hours. The reaction mixture was washed with saturated sodium sulfite aqueous solution and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=9:1 to 6:1) to give tert-butyl (4RS,6S)-2-(4-chloro-2-fluorophenyl)-3-iodo-4,6-dimethyl-6,7-dihydropyrazolo[1,5-a]pyrazine-5(4H)-carboxylate (830 mg, 1.64 mmol, 89% yield) as a yellow solid.

LC-MS (Method 11): Rₜ=1.015 min; MS (ESIpos): m/z=506.0 [M+H]⁺.

### Intermediate 541

### tert-butyl (4RS,6S)-2-(4-chloro-2-fluorophenyl)-4,6-dimethyl-3-(pyridin-4-yl)-6,7-dihydropyrazolo[1,5-a]pyrazine-5(4H)-carboxylate

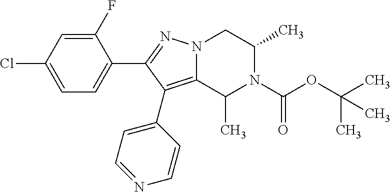 [SMILES: CC1c2c(-c3ccncc3)c(-c3ccc(Cl)cc3F)nn2C[C@H](C)N1C(=O)OC(C)(C)C]

To a solution of tert-butyl (4RS,6S)-2-(4-chloro-2-fluorophenyl)-3-iodo-4,6-dimethyl-6,7-dihydropyrazolo[1,5-a]pyrazine-5(4H)-carboxylate (830 mg, 1.64 mmol, Intermediate 540) and pyridin-4-ylboronic acid (262 mg, 2.13 mmol) in 1,4-dioxane (15 ml) and water (3 ml) mg, mmol) and (1,1-were added sodium carbonate (348 3.28 bis(diphenylphosphino) ferrocene) palladium (II) dichloride (120 mg, 0.164 mmol) at 20° C. The mixture was stirred at 90° C. for 16 hours under nitrogen atmosphere. The reaction solution was poured into water and extracted with ethyl acetate. The combined organic layers were washed with brine, dried over anhydrous sodium sulfate, filtered and concentrated in vacuo to give a residue. The residue was purified by column chromatography on silica gel (petroleum ether:ethyl acetate=4:1 to 2:1) to give tert-butyl (4RS,6S)-2-(4-chloro-2-fluorophenyl)-4,6-dimethyl-3-(pyridin-4-yl)-6,7-dihydropyrazolo[1,5-a]pyrazine-5(4H)-carboxylate (700 mg, 1.53 mmol, 93% yield) as a yellow oil.

LC-MS (Method 11): Rₜ=0.873 min; MS (ESIpos): m/z=457.2 [M+H]⁺.

### Intermediate 542

### (4RS,6S)-2-(4-chloro-2-fluorophenyl)-4,6-dimethyl-3-(pyridin-4-yl)-4,5,6,7-tetrahydropyrazolo[1,5-a]pyrazine hydrogen chloride (1/1)

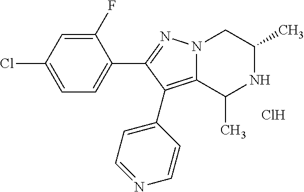 [SMILES: CC1N[C@@H](C)Cn2nc(-c3ccc(Cl)cc3F)c(-c3ccncc3)c21 ; Cl]

A solution of tert-butyl (4RS,6S)-2-(4-chloro-2-fluorophenyl)-4,6-dimethyl-3-(pyridin-4-yl)-6,7-dihydropyrazolo[1,5-a]pyrazine-5(4H)-carboxylate (700 mg, 1.53 mmol, Intermediate 541) in hydrochloric acid (15 ml, 4 M in ethyl acetate) was stirred at 20° C. for 1 hour. The reaction mixture was concentrated in vacuo to give (4RS,6S)-2-(4-chloro-2-fluorophenyl)-4,6-dimethyl-3-(pyridin-4-yl)-4,5,6,7-tetrahydropyrazolo[1,5-a]pyrazine hydrogen chloride (1/1) (500 mg, 1.27 mmol, 83% yield) as a yellow solid.

LC-MS (Method 11): Rₜ=0.645 min; MS (ESIpos): m/z=357.1 [(M−36)+H]⁺.

### Intermediate 543

### 1-[(4RS,6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one

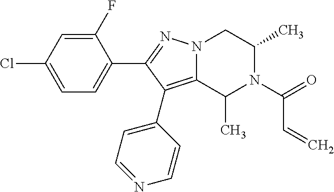 [SMILES: C=CC(=O)N1C(C)c2c(-c3ccncc3)c(-c3ccc(Cl)cc3F)nn2C[C@@H]1C]

To a solution of (4RS,6S)-2-(4-chloro-2-fluorophenyl)-4,6-dimethyl-3-(pyridin-4-yl)-4,5,6,7-tetrahydropyrazolo[1,5-a]pyrazine hydrogen chloride (1/1) (450 mg, 1.14 mmol, Intermediate 542) in dichloromethane (27 ml) were added triethylamine (347 mg, 3.40 mmol) and acryloyl chloride (104 mg, 1.14 mmol) at −10° C. The mixture was stirred at −10° C. for 1 hour. The reaction mixture was concentrated under reduced pressure to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 16%-46% B; flow 25 ml/min; temperature: RT; Detector: UV 220/254 nm] to give 1-[(4RS,6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (230 mg, 0.559 mmol, 49% yield) as a white solid.

LC-MS (Method 11): R₁=0.679 min; MS (ESIpos): m/z=411.1 [M+H]⁺.

### SYNTHESES OF EXAMPLES

### Example 1

### 1-[2-(4-fluorophenyl)-3-(pyridin-4-yl)-6,7-dihydropyrazolo[1,5-a]pyrazin-5(4H)-yl]prop-2-en-1-one

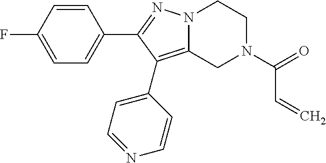 [SMILES: C=CC(=O)N1CCn2nc(-c3ccc(F)cc3)c(-c3ccncc3)c2C1]

To a solution of 2-(4-fluorophenyl)-3-(pyridin-4-yl)-4,5,6,7-tetrahydropyrazolo[1,5-a]pyrazine (50.0 mg, 170 μmol, Intermediate 3) in dry DMF (2.1 ml) was added prop-2-enoic acid (18.4 mg, 255 μmol), then N,N-diisopropylethylamine (180 μl, 1.0 mmol) followed by 2,4,6-tripropyl-1,3,5,2lambda5,4lambda5,6lambda5-trioxatriphosphinane-2,4,6-trione (solution in DMF, 162 mg, 50% purity, 255 μmol) and the mixture was stirred at rt for 25 min. The reaction mixture was treated with 100 μl water and the reactions mixture was purified by preparative HPLC (acidic conditions) to give 24 mg of the title compound (37% yield).

LC-MS (method 1): Rₜ=0.56 min; MS (ESIpos): m/z=349.1 [M+H]⁺

¹H-NMR (400 MHz, CHLOROFORM-d) δ [ppm]: 0.841 (1.21), 0.859 (1.52), 0.868 (1.41), 0.886 (2.22), 0.894 (1.51), 0.903 (1.44), 0.913 (1.84), 0.930 (0.86), 0.941 (1.38), 0.960 (2.79), 0.978 (1.39), 1.110 (0.47), 1.260 (16.00), 1.325 (1.20), 1.332 (1.27), 1.344 (1.28), 1.360 (0.92), 1.404 (0.73), 1.422 (0.69), 1.434 (0.64), 1.453 (0.68), 1.471 (0.80), 1.492 (0.63), 1.511 (0.41), 1.725 (0.68), 1.741 (0.63), 1.756 (0.46), 2.181 (1.32), 4.124 (1.29), 4.235 (0.99), 4.262 (1.14), 4.271 (1.10), 4.276 (1.06), 4.285 (0.92), 4.341 (4.10), 4.354 (5.53), 4.917 (1.79), 5.308 (0.48), 5.821 (1.66), 5.845 (1.70), 6.378 (3.57), 6.383 (3.74), 6.420 (5.81), 6.425 (5.75), 6.616 (0.58), 6.998 (6.40), 7.003 (2.46), 7.020 (12.81), 7.037 (2.74), 7.042 (7.21), 7.049 (1.16), 7.081 (6.68), 7.094 (6.76), 7.366 (6.88), 7.372 (3.22), 7.380 (7.48), 7.388 (6.92), 7.397 (3.01), 7.402 (6.08), 8.105 (4.26), 8.585 (4.37).

### Example 2

### 1-[2-(4-chloro-3-methylphenyl)-3-(pyridin-4-yl)-6,7-dihydropyrazolo[1,5-a]pyrazin-5(4H)-yl]prop-2-en-1-one

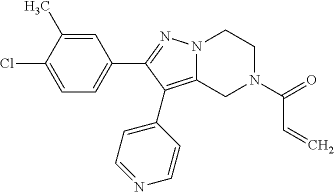 [SMILES: C=CC(=O)N1CCn2nc(-c3ccc(Cl)c(C)c3)c(-c3ccncc3)c2C1]

To a solution of 2-(4-chloro-3-methylphenyl)-3-(pyridin-4-yl)-4,5,6,7-tetrahydropyrazolo[1,5-a]pyrazine (76.0 mg, 234 μmol, Intermediate 8) in dry DMF (2.9 ml) was added prop-2-enoic acid (25.3 mg, 351 μmol), then N,N-diisopropylethylamine (240 μl, 1.4 mmol; CAS-RN: [7087-68-5]) followed by 2,4,6-tripropyl-1,3,5,2lambda⁵,4lambda⁵,6lambda⁵-trioxatriphosphinane-2,4,6-trione (solution in DMF, 223 mg, 50% purity, 351 μmol) and the mixture was stirred at rt for 25 min. The reaction mixture was treated with 200 μl water and the reactions mixture was purified by preparative HPLC (acidic conditions) to give 29 mg of the title compound (31% yield).

LC-MS (method 1): Rₜ=0.70 min; MS (ESIpos): m/z=379 [M+H]⁺

¹H-NMR (400 MHz, DMSO-d6) δ [ppm]: 2.292 (16.00), 2.323 (1.35), 2.327 (0.83), 2.331 (0.53), 2.518 (2.44), 2.523 (1.72), 2.665 (0.49), 2.669 (0.67), 2.673 (0.46), 4.084 (0.52), 4.156 (1.25), 4.234 (0.60), 4.283 (1.32), 4.835 (2.02), 4.962 (0.63), 5.772 (0.63), 5.798 (0.57), 6.157 (0.91), 6.199 (1.00), 6.924 (0.43), 6.950 (0.42), 7.068 (0.92), 7.086 (0.88), 7.181 (2.47), 7.357 (3.07), 7.377 (2.73), 7.418 (2.01), 8.550 (2.64), 8.560 (2.23).

### Example 3

### 1-[2-(4-chlorophenyl)-3-(pyridin-4-yl)-6,7-dihydropyrazolo[1,5-a]pyrazin-5(4H)-yl]prop-2-en-1-one

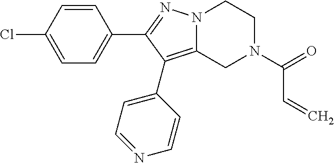 [SMILES: C=CC(=O)N1CCn2nc(-c3ccc(Cl)cc3)c(-c3ccncc3)c2C1]

In analogy to example 1 2-(4-chlorophenyl)-3-(pyridin-4-yl)-4,5,6,7-tetrahydropyrazolo[1,5-a]pyrazine (70.2 mg, 226 μmol, Intermediate 12) was used to prepare 33 mg of the title compound (38% yield) after purification by preparative HPLC (basic conditions).

LC-MS (Method 1): Rₜ=0.7 min; MS (ESIpos): m/z=365 [M+H]⁺

¹H-NMR (400 MHz, DMSO-d6) δ [ppm]: 2.074 (1.20), 2.331 (1.04), 2.337 (0.47), 2.518 (5.51), 2.523 (3.82), 2.673 (1.04), 2.678 (0.45), 4.086 (1.08), 4.157 (2.65), 4.241 (1.23), 4.289 (2.80), 4.835 (4.43), 4.964 (1.35), 5.772 (1.34), 5.797 (1.21), 6.157 (1.96), 6.199 (2.16), 6.923 (0.89), 6.951 (0.87), 6.965 (0.81), 6.991 (0.63), 7.182 (5.34), 7.348 (3.81), 7.368 (5.64), 7.415 (16.00), 7.420 (4.41), 7.431 (3.45), 7.436 (8.11), 8.554 (5.78), 8.565 (4.67).

### Example 4

### 1-[2-(3-fluorophenyl)-3-(pyridin-4-yl)-6,7-dihydropyrazolo[1,5-a]pyrazin-5(4H)-yl]prop-2-en-1-one

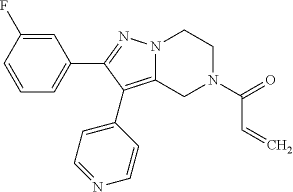 [SMILES: C=CC(=O)N1CCn2nc(-c3cccc(F)c3)c(-c3ccncc3)c2C1]

Using an analogous method as described for example 1, 2-(3-fluorophenyl)-3-(pyridin-4-yl)-4,5,6,7-tetrahydropyrazolo[1,5-a]pyrazine (131 mg, 444 μmol, Intermediate 16) as the starting material, 45 mg of the title compound (28% yield) were prepared after purification by preparative HPLC (basic conditions).

LC-MS (Method 1): Rₜ=0.6 min; MS (ESIpos): m/z=349.1 [M+H]⁺

¹H-NMR (400 MHz, DMSO-d6) δ [ppm]: 1.232 (0.63), 2.518 (13.63), 2.523 (8.59), 2.660 (1.02), 4.087 (2.47), 4.161 (5.92), 4.297 (6.31), 4.674 (0.47), 4.830 (9.79), 4.961 (3.00), 5.773 (3.05), 5.799 (2.80), 6.159 (4.36), 6.201 (4.85), 6.556 (0.41), 6.925 (1.97), 6.952 (1.95), 6.967 (1.78), 6.992 (1.45), 7.139 (8.25), 7.150 (11.42), 7.153 (11.73), 7.157 (11.37), 7.173 (9.26), 7.182 (10.67), 7.196 (15.82), 7.200 (16.00), 7.354 (1.23), 7.364 (5.21), 7.369 (1.96), 7.379 (6.20), 7.383 (7.64), 7.399 (8.43), 7.404 (4.62), 7.419 (3.75), 8.564 (13.20), 8.632 (0.86), 8.647 (0.72), 9.653 (1.32).

### Example 5

### 1-[2-(3-methoxyphenyl)-3-(pyridin-4-yl)-6,7-dihydropyrazolo[1,5-a]pyrazin-5(4H)-yl]prop-2-en-1-one

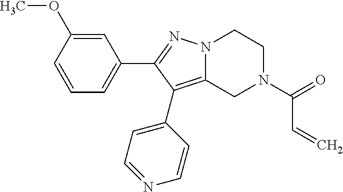 [SMILES: C=CC(=O)N1CCn2nc(-c3cccc(OC)c3)c(-c3ccncc3)c2C1]

Using an analogous method as described for example 1, 2-(3-methoxyphenyl)-3-(pyridin-4-yl)-4,5,6,7-tetrahydropyrazolo[1,5-a]pyrazine (98.0 mg, 320 μmol, Intermediate 20) as the starting material, 35 mg of the title compound (30% yield) were prepared after purification by preparative HPLC (basic conditions).

LC-MS (Method 1): Rₜ=0.5 min; MS (ESIpos): m/z=361.1 [M+H]⁺

¹H-NMR (400 MHz, DMSO-d6) δ [ppm]: 2.518 (1.73), 2.523 (1.15), 3.668 (16.00), 4.156 (0.74), 4.287 (0.79), 4.827 (1.24), 6.158 (0.52), 6.200 (0.58), 6.889 (2.67), 6.905 (2.14), 6.910 (1.10), 7.185 (1.49), 7.230 (0.99), 7.250 (1.30), 7.271 (0.64), 8.544 (1.63), 8.554 (1.33).

### Example 6

### N-{4-[2-(4-fluorophenyl)-5-{(2E)-4-[methyl(propan-2-yl)amino]but-2-enoyl}-4,5,6,7-tetrahydropyrazolo[1,5-a]pyrazin-3-yl]pyridin-2-yl}cyclopropanecarboxamide

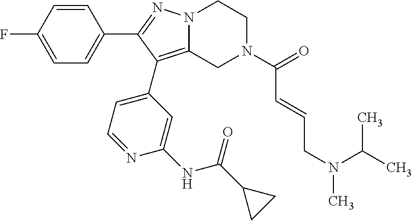 [SMILES: CC(C)N(C)C/C=C/C(=O)N1CCn2nc(-c3ccc(F)cc3)c(-c3ccnc(NC(=O)C4CC4)c3)c2C1]

Using an analogous method as described for example 1, N-{4-[2-(4-fluorophenyl)-4,5,6,7-tetrahydropyrazolo[1,5-a]pyrazin-3-yl]pyridin-2-yl}cyclopropanecarboxamide (100 mg, 265 μmol, Intermediate 22) and (2E)-4-[methyl(propan-2-yl)amino]but-2-enoic acid (62.5 mg, 397 μmol) as the starting materials, 8 mg of the title compound (5% yield) were prepared after purification by preparative HPLC (basic conditions).

LC-MS (Method 1): Rₜ=0.7 min; MS (ESIpos): m/z=517.1 [M+H]⁺

¹H-NMR (400 MHz, DMSO-d6) δ [ppm]: 0.788 (8.36), 0.920 (5.17), 0.938 (14.91), 0.955 (16.00), 1.023 (2.33), 1.040 (2.31), 1.112 (1.71), 1.128 (1.64), 1.232 (0.52), 1.991 (1.92), 2.006 (2.51), 2.035 (3.06), 2.095 (12.38), 2.332 (1.74), 2.518 (12.54), 2.523 (7.79), 2.673 (1.86), 2.712 (2.09), 2.750 (1.38), 2.767 (1.64), 2.783 (1.30), 2.833 (2.27), 3.154 (4.39), 3.168 (3.45), 4.073 (1.33), 4.140 (2.21), 4.254 (2.43), 4.768 (3.00), 4.795 (1.75), 4.829 (1.45), 4.834 (1.53), 4.840 (1.52), 4.844 (1.50), 4.907 (1.42), 6.055 (0.52), 6.068 (0.84), 6.095 (0.63), 6.102 (0.57), 6.107 (0.90), 6.617 (2.10), 6.628 (2.31), 6.669 (1.57), 6.753 (3.07), 6.881 (0.78), 6.920 (0.74), 7.162 (4.75), 7.184 (10.06), 7.207 (5.57), 7.375 (6.18), 7.381 (3.19), 7.389 (7.07), 7.397 (6.27), 7.406 (2.58), 7.411 (5.16), 7.547 (0.45), 7.565 (0.51), 7.573 (0.48), 7.595 (0.64), 7.613 (0.56), 7.625 (0.64), 7.989 (2.53), 8.234 (2.57), 10.858 (4.55)

### Example 7

### N-{4-[2-(4-fluorophenyl)-5-(prop-2-enoyl)-4,5,6,7-tetrahydropyrazolo[1,5-a]pyrazin-3-yl]pyridin-2-yl}cyclopropanecarboxamide

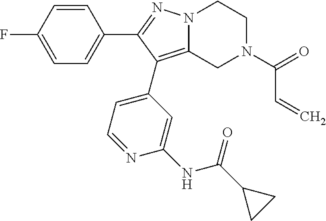 [SMILES: C=CC(=O)N1CCn2nc(-c3ccc(F)cc3)c(-c3ccnc(NC(=O)C4CC4)c3)c2C1]

Using an analogous method as described for example 1, N-{4-[2-(4-fluorophenyl)-4,5,6,7-tetrahydropyrazolo[1,5-a]pyrazin-3-yl]pyridin-2-yl}cyclopropanecarboxamide (30.0 mg, 79.5 μmol, Intermediate 22) and prop-2-enoic acid (8.59 mg, 119 μmol) as the starting materials, 22 mg of the title compound (58% yield) were prepared after purification by preparative HPLC (basic conditions).

LC-MS (Method 1): Rₜ=0.86 min; MS (ESIpos): m/z=432.1 [M+H]⁺

¹H-NMR (400 MHz, CHLOROFORM-d) δ [ppm]: 2.492 (5.55), 2.496 (11.71), 2.501 (16.00), 2.505 (11.61), 2.509 (5.48), 2.518 (0.51), 3.332 (9.04).

### Example 8

### N-(4-{5-[(2E)-4-(dimethylamino)but-2-enoyl]-2-(4-fluorophenyl)-4,5,6,7-tetrahydropyrazolo[1,5-a]pyrazin-3-yl}pyridin-2-yl)cyclopropanecarboxamide

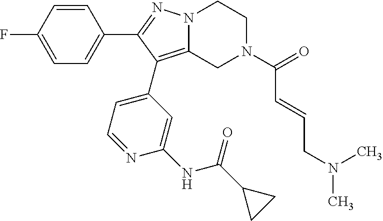 [SMILES: CN(C)C/C=C/C(=O)N1CCn2nc(-c3ccc(F)cc3)c(-c3ccnc(NC(=O)C4CC4)c3)c2C1]

N-{4-[2-(4-fluorophenyl)-4,5,6,7-tetrahydropyrazolo[1,5-a]pyrazin-3-yl]pyridin-2-yl}cyclopropanecarboxamide (43.2 mg, 114 μmol, Intermediate 22) was dissolved in DMF (2.0 ml), then 2,4,6-Tripropyl-1,3,5,2,4,6-trioxatriphosphorinane-2,4,6-trioxide solution (100 μl, 50% purity in DMF, 170 μmol), N,N-diisopropylethylamine (120 μl, 690 μmol; CAS-RN: [7087-68-5]) and (2E)-4-(dimethylamino)but-2-enoic acid (17.7 mg, 137 μmol) were added, and the mixture was stirred at rt over night. Water (0.1 ml) was added the mixture was purified by reverse phase preparative HPLC (acidic) yielding the title compound (21 mg, 95% purity, 34% yield)

LC-MS (Method 3): Rₜ=0.77 min; MS (ESIpos): m/z=489.4 [M+H]⁺

¹H-NMR (400 MHz, DMSO-d6) δ [ppm]: 0.784 (8.94), 0.915 (13.46), 0.932 (10.87), 0.951 (3.43), 0.982 (1.36), 0.999 (1.47), 1.026 (1.34), 1.044 (1.39), 1.107 (16.00), 1.231 (1.25), 1.474 (12.65), 2.005 (2.30), 2.323 (3.43), 2.327 (4.25), 2.331 (3.67), 2.522 (11.67), 2.665 (2.53), 2.669 (3.26), 2.673 (2.48), 3.703 (5.89), 4.023 (6.24), 4.089 (2.90), 4.215 (2.87), 4.784 (3.69), 4.920 (1.61), 6.673 (1.04), 6.760 (4.13), 6.772 (4.10), 7.163 (5.60), 7.185 (11.82), 7.207 (6.56), 7.371 (5.01), 7.385 (6.14), 7.406 (3.97), 7.991 (4.58), 8.088 (0.65), 8.239 (4.00), 8.252 (3.58), 10.866 (4.98).

### Example 9

### N-{4-[2-(4-fluorophenyl)-5-(oxirane-2-carbonyl)-4,5,6,7-tetrahydropyrazolo[1,5-a]pyrazin-3-yl]pyridin-2-yl}cyclopropanecarboxamide

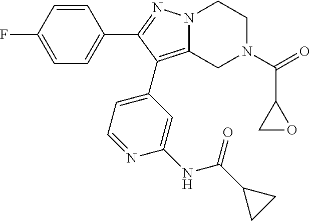 [SMILES: O=C(Nc1cc(-c2c(-c3ccc(F)cc3)nn3c2CN(C(=O)C2CO2)CC3)ccn1)C1CC1]

N-{4-[2-(4-fluorophenyl)-4,5,6,7-tetrahydropyrazolo[1,5-a]pyrazin-3-yl]pyridin-2-yl}cyclopropanecarboxamide (46.5 mg, 123 μmol, Intermediate 22) was dissolved in DMF (2 ml), then N,N-diisopropylethylamine (130 μl, 740 μmol; CAS-RN: [7087-68-5]), 2,4,6-Tripropyl-1,3,5,2,4,6-trioxatriphosphorinane-2,4,6-trioxide solution (50% purity in DMF, 118 mg, 185 μmol) and potassium oxirane-2-carboxylate (18.7 mg, 148 μmol, CAS-RN: [51877-54-4]) were added, and the mixture stirred for 4 h at rt. Water (0.1 ml) was added and the mixture purified by purified by reverse phase preparative HPLC (acidic), yielding the title compound (11.5 mg, 90% purity, 19% yield).

LC-MS (Method 3): Rₜ=0.89 min; MS (ESIpos): m/z=448 [M+H]⁺

¹H-NMR (400 MHz, DMSO-d6) δ [ppm]: 0.772 (16.00), 0.779 (9.41), 0.791 (9.80), 0.822 (0.80), 0.840 (1.02), 0.848 (0.80), 0.865 (0.78), 0.885 (0.67), 0.903 (1.26), 0.915 (1.16), 0.932 (0.83), 1.026 (0.41), 1.230 (1.88), 1.299 (0.51), 1.351 (0.73), 1.418 (1.05), 1.905 (2.27), 1.993 (2.69), 2.140 (0.79), 2.193 (2.68), 2.336 (1.33), 2.518 (15.92), 2.523 (11.91), 2.679 (1.33), 2.767 (0.80), 2.782 (1.06), 2.812 (2.69), 2.819 (2.63), 2.829 (3.47), 2.834 (2.97), 2.845 (0.98), 2.888 (1.04), 2.900 (1.04), 2.914 (0.74), 2.945 (3.27), 2.956 (3.62), 2.961 (3.90), 2.971 (2.65), 3.930 (1.41), 3.965 (0.60), 3.990 (1.11), 4.008 (1.15), 4.031 (4.23), 4.038 (5.03), 4.041 (4.87), 4.048 (4.17), 4.146 (1.89), 4.159 (1.99), 4.173 (1.30), 4.204 (3.27), 4.218 (3.99), 4.228 (3.90), 4.262 (1.79), 4.274 (1.57), 4.328 (3.50), 4.340 (2.99), 4.734 (9.33), 4.745 (4.69), 4.926 (0.55), 4.969 (1.55), 5.002 (1.73), 5.020 (0.93), 5.043 (0.54), 6.596 (5.13), 6.751 (3.10), 6.754 (3.17), 6.763 (3.21), 6.767 (3.10), 6.827 (1.18), 6.839 (1.23), 7.125 (1.46), 7.161 (7.06), 7.183 (14.56), 7.201 (5.05), 7.206 (8.71), 7.223 (4.23), 7.245 (2.19), 7.375 (5.92), 7.389 (7.37), 7.397 (5.60), 7.411 (4.36), 7.480 (0.68), 7.499 (0.47), 7.564 (0.74), 7.775 (1.21), 7.797 (1.78), 7.811 (1.78), 7.921 (0.74), 7.924 (0.85), 7.942 (0.81), 7.971 (7.56), 8.087 (1.48), 8.234 (4.69), 8.247 (5.17), 10.789 (0.42), 10.849 (5.00).

### Example 10

### 1-[3-(2-aminopyridin-4-yl)-2-(4-fluorophenyl)-6,7-dihydropyrazolo[1,5-a]pyrazin-5(4H)-yl]prop-2-en-1-one

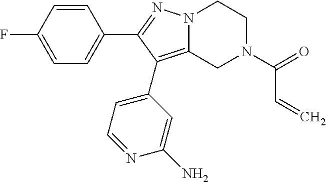 [SMILES: C=CC(=O)N1CCn2nc(-c3ccc(F)cc3)c(-c3ccnc(N)c3)c2C1]

4-[2-(4-fluorophenyl)-4,5,6,7-tetrahydropyrazolo[1,5-a]pyrazin-3-yl]pyridin-2-amine (70.0 mg, 226 μmol, Intermediate 24) was added to a reaction vessel, followed by DMF (2.8 ml), prop-2-enoic acid (20 μl, 290 μmol, CAS-RN: [79-10-7]) N,N-diisopropylethylamine (240 μl, 1.4 mmol; CAS-RN: [7087-68-5]) and lastly 2,4,6-Tripropyl-1,3,5,2,4,6-trioxatriphosphorinane-2,4,6-trioxide solution (200 μl, 50% purity in DMF, 340 μmol) was added dropwise under nitrogen. The mixture was stirred for 15 minutes at rt, followed by the addition of water (10 ml), and the mixture was extracted with EtOAc (60 ml). The organic phase was washed with NaHCO: solution, followed by saturated NH₄Cl solution, dried over Na₂SO₄, filtered, concentrated under reduced pressure and purified by preparative reverse phase HPLC yielding the title compound (12 mg, 90% purity, 13% yield)

LC-MS (Method 3): Rₜ=0.69 min; MS (ESIpos): m/z=364 [M+H]⁺

¹H-NMR (400 MHz, DMSO-d6) δ [ppm]: 2.318 (0.87), 2.518 (10.09), 2.523 (7.09), 2.660 (0.90), 2.888 (0.48), 4.079 (1.19), 4.148 (3.55), 4.254 (3.74), 4.761 (5.89), 4.853 (1.52), 5.768 (1.92), 5.794 (1.73), 5.946 (5.32), 6.162 (1.95), 6.204 (2.20), 6.249 (12.80), 6.286 (0.83), 6.920 (1.05), 6.946 (1.10), 6.961 (1.05), 6.988 (0.84), 7.156 (0.81), 7.163 (7.72), 7.169 (2.66), 7.180 (3.45), 7.185 (16.00), 7.191 (3.42), 7.202 (2.90), 7.208 (9.06), 7.215 (1.11), 7.403 (4.76), 7.418 (5.91), 7.424 (5.27), 7.438 (3.85), 7.862 (7.97), 7.876 (7.50), 8.158 (6.48).

### Example 11

### 1-[2-(4-fluorophenyl)-3-(2-methoxypyridin-4-yl)-6,7-dihydropyrazolo[1,5-a]pyrazin-5(4H)-yl]prop-2-en-1-one

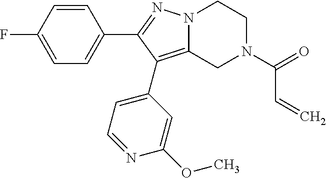 [SMILES: C=CC(=O)N1CCn2nc(-c3ccc(F)cc3)c(-c3ccnc(OC)c3)c2C1]

2-(4-fluorophenyl)-3-(2-methoxypyridin-4-yl)-4,5,6,7-tetrahydropyrazolo[1,5-a]pyrazine (98.7 mg, 304 μmol, Intermediate 26) was dissolved in DMF (4.0 ml), then N,N-diisopropylethylamine (320 μl, 1.8 mmol; CAS-RN: [7087-68-5]), 2,4,6-Tripropyl-1,3,5,2,4,6-trioxatriphosphorinane-2,4,6-trioxide solution (50% purity in DMF, 145 mg, 456 μmol) and prop-2-enoic acid (31 μl, 460 μmol; CAS-RN: [79-10-7]) were added, and the mixture stirred over night at rt. A few drops of water were added, the mixture was concentrated under reduced pressure and purified by reverse phase preparative HPLC (acidic), yielding the title compound (13.2 mg, 95% purity, 11% yield)

LC-MS (Method 3): Rₜ=1.08 min; MS (ESIpos): m/z=379.2 [M+H]⁺

¹H-NMR (400 MHz, METHANOL-d4) δ [ppm]: 3.872 (0.68), 3.892 (16.00), 3.961 (3.32), 4.200 (0.80), 4.213 (1.73), 4.226 (1.46), 4.317 (0.62), 4.328 (0.62), 4.923 (1.57), 4.962 (0.44), 6.623 (1.80), 6.625 (1.84), 6.737 (1.67), 6.741 (1.53), 6.750 (1.68), 6.754 (1.59), 7.066 (1.27), 7.071 (0.45), 7.083 (0.77), 7.088 (2.71), 7.105 (0.89), 7.110 (1.62), 7.395 (0.94), 7.409 (1.13), 7.416 (0.99), 7.430 (0.77), 8.062 (1.66), 8.064 (1.74), 8.078 (1.68).

### Example 12

### 1-[2-(4-fluorophenyl)-3-(3-methoxypyridin-4-yl)-6,7-dihydropyrazolo[1,5-a]pyrazin-5(4H)-yl]prop-2-en-1-one

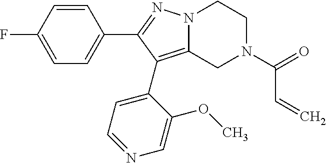 [SMILES: C=CC(=O)N1CCn2nc(-c3ccc(F)cc3)c(-c3ccncc3OC)c2C1]

2-(4-fluorophenyl)-3-(3-methoxypyridin-4-yl)-4,5,6,7-tetrahydropyrazolo[1,5-a]pyrazine (95.0 mg, 293 μmol, Intermediate 28) was dissolved in DMF (4.0 ml), then N,N-diisopropylethylamine (310 μl, 1.8 mmol; CAS-RN: [7087-68-5]), 2,4,6-Tripropyl-1,3,5,2,4,6-trioxatriphosphorinane-2,4,6-trioxide solution (260 μl, 50% purity in DMF, 440 μmol) and prop-2-enoic acid (30 μl, 440 μmol; CAS-RN: [79-10-7]) were added, and the mixture stirred over night at rt. A few drops of water were added and the mixture was concentration under reduced pressure followed by reverse phase preparative HPLC (acidic), yielding the title compound (4.90 mg, 79% purity, 3% yield).

LC-MS (Method 3): Rₜ=0.76 min; MS (ESIpos): m/z=379 [M+H]⁺

### Example 13

### 1-[2-(4-fluorophenyl)-3-(pyridin-4-yl)-6,7-dihydropyrazolo[1,5-a]pyrazin-5(4H)-yl]prop-2-yn-1-one

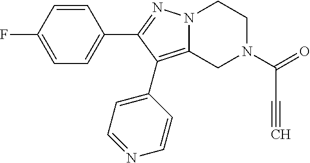 [SMILES: C#CC(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (50.0 mg, 170 μmol, Intermediate 3) was added to a reaction vessel, followed by DMF (2.1 ml), prop-2-ynoic acid (16 μl, 250 μmol, CAS-RN: [471-25-0]) N,N-diisopropylethylamine (180 μl, 1.0 mmol; CAS-RN: [7087-68-5]) and lastly 2,4,6-Tripropyl-1,3,5,2,4,6-trioxatriphosphorinane-2,4,6-trioxide solution (150 μl, 50% purity in DMF, 250 μmol) was added dropwise under nitrogen. The reaction was stirred for 15 minutes at rt, then water (10 ml) was added, and the mixture extracted with EtOAc (60 ml), the organic phase dried over Na₂SO₄, filtered and concentrated under reduced pressure. Purification by reverse phase preparative HPLC yielded the title compound (6.00 mg, 95% purity, 10% yield) LC-MS (Method 3): Rₜ=0.70 min; MS (ESIpos): m/z=347 [M+H]⁺

¹H-NMR (400 MHz, DMSO-d6) δ [ppm]: 1.027 (0.62), 1.042 (0.64), 1.052 (0.40), 2.318 (1.29), 2.518 (16.00), 2.523 (10.64), 2.678 (1.26), 4.067 (0.76), 4.081 (1.50), 4.095 (1.10), 4.226 (1.09), 4.241 (1.76), 4.259 (2.75), 4.273 (2.22), 4.331 (2.15), 4.344 (2.60), 4.357 (1.11), 4.667 (7.10), 4.747 (13.32), 4.810 (7.93), 5.056 (4.01), 7.149 (6.12), 7.154 (3.56), 7.161 (4.19), 7.164 (7.34), 7.174 (2.28), 7.178 (6.83), 7.184 (1.57), 7.195 (1.81), 7.201 (9.48), 7.207 (1.68), 7.218 (1.56), 7.223 (5.18), 7.231 (0.57), 7.353 (1.91), 7.359 (3.74), 7.367 (2.38), 7.372 (4.32), 7.375 (3.00), 7.381 (3.15), 7.389 (2.32), 7.394 (2.59), 8.538 (5.93), 8.542 (3.32), 8.553 (6.71), 8.563 (1.77), 8.567 (2.88).

### Example 14

### 5-(ethenesulfonyl)-2-(4-fluorophenyl)-3-(pyridin-4-yl)-4,5,6,7-tetrahydropyrazolo[1,5-a]pyrazine

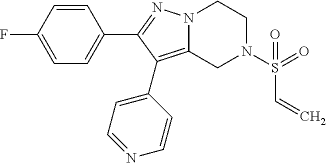 [SMILES: C=CS(=O)(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (60.0 mg, 204 μmol, Intermediate 3) was added to a reaction vessel, followed by DMF (1.5 ml), triethylamine (99 μl, 710 μmol; CAS-RN: [121-44-8]) and then the dropwise addition of 2-chloroethane-1-sulfonyl chloride (21 μl, 200 μmol, CAS-RN: [1622-32-8]) and the mixture stirred overnight under nitrogen. Water (1 ml) was added, followed by a NaHCO; solution (saturated. aqueous. 10 ml) and the mixture extracted with EtOAc (60 ml). The organic phase was dried over Na₂SO₄, and concentrated under reduced pressure. Purification by preparative HPLC (acidic) yielded the title compound (20.0 mg, 95% purity, 24% yield).

LC-MS (Method 3): Rₜ=0.76 min; MS (ESIpos): m/z=385 [M+H]⁺

¹H-NMR (400 MHz, DMSO-d6) δ [ppm]: 2.331 (2.81), 2.336 (1.29), 2.518 (16.00), 2.523 (10.50), 2.673 (2.78), 3.719 (2.87), 3.733 (4.61), 3.747 (3.03), 4.296 (3.07), 4.311 (4.79), 4.323 (2.80), 4.489 (13.02), 6.210 (6.76), 6.216 (6.59), 6.235 (7.15), 6.257 (7.14), 6.956 (4.12), 6.980 (4.01), 6.997 (4.24), 7.022 (3.38), 7.138 (10.31), 7.143 (6.18), 7.150 (6.20), 7.153 (10.34), 7.174 (4.60), 7.180 (1.66), 7.191 (1.98), 7.197 (9.89), 7.202 (1.95), 7.214 (1.71), 7.219 (5.89), 7.227 (0.69), 7.346 (0.77), 7.354 (5.61), 7.360 (2.20), 7.368 (6.05), 7.376 (5.14), 7.384 (1.84), 7.390 (4.45), 8.539 (10.02), 8.543 (5.82), 8.550 (5.71), 8.555 (9.36).

### Example 15

### (2E)-4-(dimethylamino)-1-[2-(4-fluorophenyl)-3-(pyridin-4-yl)-6,7-dihydropyrazolo[1,5-a]pyrazin-5(4H)-yl]but-2-en-1-one

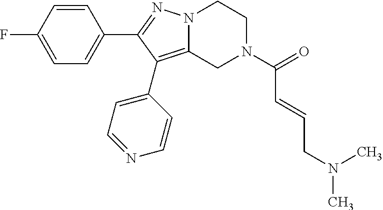 [SMILES: CN(C)C/C=C/C(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (50.0 mg, 170 μmol, Intermediate 3) was added to a reaction vessel, followed by DMF (2.1 ml), (2E)-4-(dimethylamino)but-2-enoic acid-hydrogen chloride (1/1) (42.2 mg, 255 μmol, CAS-RN: [848133-35-7]) N,N-diisopropylethylamine (180 μl, 1.0 mmol; CAS-RN: [7087-68-5]) and lastly 2,4,6-Tripropyl-1,3,5,2,4,6-trioxatriphosphorinane-2,4,6-trioxide solution (150 μl, 50% purity in DMF, 250 μmol) was added dropwise under nitrogen. The mixture was stirred for 15 minutes at rt, then added into water (10 ml), and the aqueous phase extracted with EtOAc (60 ml), the organic phase dried over Na₂SO₄, and concentrated under reduced pressure. Purification by preparative HPLC (acidic) yielded the title compound (15.0 mg, 95% purity, 21% yield)

LC-MS (Method 3): Rₜ=0.56 min; MS (ESIpos): m/z=406 [M+H]⁺

¹H-NMR (400 MHz, DMSO-d6) δ [ppm]: 2.094 (4.48), 2.154 (16.00), 2.331 (2.16), 2.336 (1.02), 2.518 (12.82), 2.523 (8.54), 2.673 (2.11), 2.678 (0.99), 3.048 (3.84), 3.060 (3.44), 4.080 (1.23), 4.138 (3.03), 4.274 (3.27), 4.823 (4.38), 4.948 (1.26), 6.645 (1.99), 6.683 (1.68), 6.742 (1.69), 6.779 (0.73), 7.155 (6.16), 7.165 (7.22), 7.174 (9.08), 7.196 (14.01), 7.214 (2.79), 7.219 (7.93), 7.353 (4.12), 7.367 (5.18), 8.173 (0.47), 8.539 (8.44), 8.553 (7.92).

### Example 16

### 1-[2-(4-fluorophenyl)-3-(pyridin-4-yl)-6,7-dihydropyrazolo[1,5-a]pyrazin-5(4H)-yl]but-2-yn-1-one

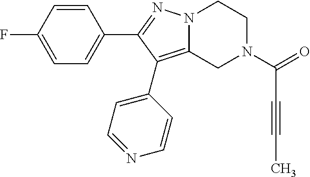 [SMILES: CC#CC(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (50.0 mg, 170 μmol, Intermediate 3) was added to a reaction vessel, followed by DMF (2.1 ml), but-2-ynoic acid (21.4 mg, 255 μmol, CAS-RN: [590-93-2]) N,N-diisopropylethylamine (180 μl, 1.0 mmol; CAS-RN: [7087-68-5]) and lastly 2,4,6-Tripropyl-1,3,5,2,4,6-trioxatriphosphorinane-2,4,6-trioxide solution (150 μl, 50% purity in DMF, 250 μmol) was added dropwise under nitrogen. The mixture was stirred for 15 minutes at rt, then added to water (10 ml), and the mixture extracted with EtOAc (60 ml), the organic layer dried over Na₂SO₄, filtered and concentrated under reduced pressure. Purification by preparative HPLC (acidic) yielded the title compound (18.0 mg, 95% purity, 28% yield).

LC-MS (Method 3): Rₜ=0.75 min; MS (ESIpos): m/z=361 [M+H]⁺

¹H-NMR (400 MHz, DMSO-d6) δ [ppm]: 1.984 (7.30), 2.083 (16.00), 2.327 (1.38), 2.331 (0.96), 2.337 (0.43), 2.518 (5.63), 2.523 (3.80), 2.669 (1.38), 2.673 (0.94), 2.678 (0.42), 4.044 (0.47), 4.058 (0.91), 4.072 (0.63), 4.204 (0.67), 4.219 (0.97), 4.233 (1.09), 4.248 (1.89), 4.262 (1.66), 4.304 (1.70), 4.318 (1.86), 4.330 (0.73), 4.792 (5.84), 5.024 (2.41), 7.147 (4.22), 7.151 (2.52), 7.158 (2.60), 7.162 (4.78), 7.168 (1.37), 7.177 (3.60), 7.183 (1.17), 7.195 (1.32), 7.200 (6.00), 7.205 (1.17), 7.217 (1.10), 7.222 (3.52), 7.350 (1.24), 7.358 (2.55), 7.363 (2.07), 7.372 (3.52), 7.381 (2.50), 7.386 (1.11), 7.389 (0.93), 7.395 (1.88), 8.534 (3.48), 8.538 (2.17), 8.545 (2.19), 8.549 (3.37), 8.557 (1.62), 8.560 (0.99), 8.568 (0.92), 8.572 (1.38).

### Example 17

### 2-fluoro-1-[2-(4-fluorophenyl)-3-(pyridin-4-yl)-6,7-dihydropyrazolo[1,5-a]pyrazin-5(4H)-yl]prop-2-en-1-one

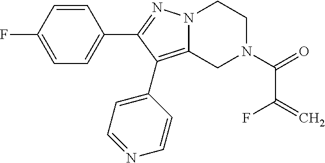 [SMILES: C=C(F)C(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (50.0 mg, 170 μmol, Intermediate 3) was added to a reaction vessel, followed by DMF (2.1 ml), 2-fluoroprop-2-enoic acid (19 μl, 250 μmol, CAS-RN: [430-99-9]), N,N-diisopropylethylamine (180 μl, 1.0 mmol; CAS-RN: [7087-68-5]) and lastly 2,4,6-Tripropyl-1,3,5,2,4,6-trioxatriphosphorinane-2,4,6-trioxide solution (150 μl, 50% purity, 250 μmol) was added dropwise under nitrogen. The mixture was stirred for 15 minutes at rt, then water (10 ml) was added, and the mixture extracted with EtOAc (60 ml), the organic layer dried over Na₂SO₄, filtered and concentrated under reduced pressure. Purification by preparative HPLC (acidic) yielded the title compound (10.0 mg, 95% purity, 15% yield).

LC-MS (Method 3): Rₜ=0.75 min; MS (ESIpos): m/z=367 [M+H]⁺

¹H-NMR (400 MHz, DMSO-d6) δ [ppm]: 2.332 (2.51), 2.336 (1.11), 2.518 (16.00), 2.523 (10.46), 2.678 (1.10), 4.086 (1.25), 4.098 (2.33), 4.112 (1.56), 4.316 (1.40), 4.846 (0.92), 5.297 (1.03), 5.307 (1.28), 5.373 (0.73), 5.425 (2.76), 7.154 (4.56), 7.158 (2.89), 7.165 (2.89), 7.169 (4.71), 7.179 (2.51), 7.184 (1.00), 7.201 (5.47), 7.218 (0.94), 7.223 (3.10), 7.354 (2.54), 7.360 (1.14), 7.368 (2.83), 7.376 (2.39), 7.385 (0.94), 7.390 (2.01), 8.544 (4.26), 8.548 (2.75), 8.555 (2.68), 8.560 (4.08).

### Example 18

### (2E)-1-[2-(4-fluorophenyl)-3-(pyridin-4-yl)-6,7-dihydropyrazolo[1,5-a]pyrazin-5(4H)-yl]-4-[methyl(propan-2-yl)amino]but-2-en-1-one

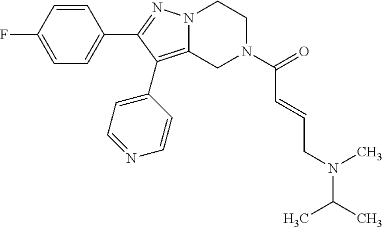 [SMILES: CC(C)N(C)C/C=C/C(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (50.0 mg, 170 μmol, Intermediate 3) was added to a reaction vessel, followed by DMF (2.1 ml), (2E)-4-[methyl(propan-2-yl)amino]but-2-enoic acid (40.1 mg, 255 μmol, CAS-RN: [1130553-97-7]), N,N-diisopropylethylamine (180 μl, 1.0 mmol; CAS-RN: [7087-68-5]) and lastly 2,4,6-Tripropyl-1,3,5,2,4,6-trioxatriphosphorinane-2,4,6-trioxide solution, (150 μl, 50% purity in DMF, 250 μmol, CAS-RN: [68957-94-8]) was added dropwise under nitrogen. The mixture was stirred for 15 minutes at rt, then water (10 ml) was added, and the mixture extracted with EtOAc (60 ml). The organic phase was dried over Na₂SO₄, filtered and concentrated under reduced pressure. Purification by preparative HPLC (acidic) yielded the title compound (4.00 mg, 90% purity, 5% yield)

LC-MS (Method 3): Rₜ=0.58 min; MS (ESIpos): m/z=434 [M+H]⁺

¹H-NMR (400 MHz, DMSO-d6) δ [ppm]: 0.910 (4.75), 0.954 (11.34), 2.043 (2.38), 2.111 (7.55), 2.318 (1.05), 2.518 (13.84), 2.523 (9.05), 2.660 (1.07), 2.786 (1.40), 3.167 (4.06), 4.133 (3.25), 4.274 (3.48), 4.819 (4.30), 4.933 (1.40), 6.636 (2.19), 6.676 (1.52), 6.753 (1.53), 6.791 (0.79), 7.154 (7.53), 7.166 (8.67), 7.174 (9.95), 7.197 (16.00), 7.219 (8.98), 7.352 (4.87), 7.367 (6.29), 7.387 (3.82), 8.155 (1.53), 8.539 (8.58), 8.552 (8.26).

### Example 19

### 1-[2-(4-fluoroanilino)-3-(pyridin-4-yl)-6,7-dihydropyrazolo[1,5-a]pyrazin-5(4H)-yl]prop-2-en-1-one

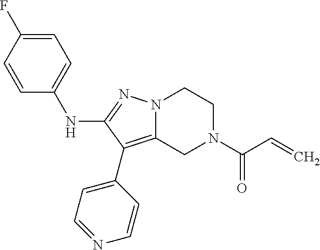 [SMILES: C=CC(=O)N1CCn2nc(Nc3ccc(F)cc3)c(-c3ccncc3)c2C1]

To a stirred solution of N-(4-fluorophenyl)-3-(pyridin-4-yl)-4,5,6,7-tetrahydropyrazolo[1,5-a]pyrazin-2-amine (23.0 mg, 74.3 μmol) in MeCN (1 ml) HATU (33.9 mg, 89.2 μmol), triethylamine (31 μl, 220 μmol) and prop-2-enoic acid (5.6 μl, 82 μmol) were added at rt. After 2 h, water (1 ml) was added and the mixture was extracted with EtOAc twice. The combined organic layers were dried over sodium sulfate, filtered and concentrated under reduced pressure. The crude product was purified by silica gel column chromatography (eluent: DCM/MeOH 10:1) yielding 8.80 mg (93% purity, 30% yield) of the title compound.

LC-MS (Method 4): Rₜ=0.89 min; MS (ESIpos): m/z=364 [M+H]⁺

¹H NMR (500 MHz, DMSO-d₆) δ ppm 3.93-4.29 (m, 4H) 4.83-5.06 (m, 2H) 5.67-5.90 (m, 1H) 6.08-6.25 (m, 1H) 6.78-7.10 (m, 3H) 7.15-7.26 (m, 2H) 7.29-7.44 (m, 2H) 7.92-8.12 (m, 1H) 8.45-8.63 (m, 2H)

### Example 20

### 1-[3-(2-anilinopyridin-4-yl)-2-(4-fluorophenyl)-6,7-dihydropyrazolo[1,5-a]pyrazin-5(4H)-yl]prop-2-en-1-one

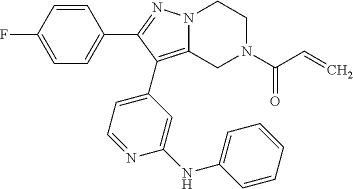 [SMILES: C=CC(=O)N1CCn2nc(-c3ccc(F)cc3)c(-c3ccnc(Nc4ccccc4)c3)c2C1]

trifluoroacetic acid-4-[2-(4-fluorophenyl)-4,5,6,7-tetrahydropyrazolo[1,5-a]pyrazin-3-yl]-N-phenylpyridin-2-amine (1/1) (42.0 mg, 84.1 μmol, Intermediate 34) was dissolved in 1 ml DMF, then 2,4,6-Tripropyl-1,3,5,2,4,6-trioxatriphosphorinane-2,4,6-trioxide solution in DMF (80.3 mg, 50% purity, 126 μmol), N,N-diisopropylethylamine (88 μl, 500 μmol; CAS-RN: [7087-68-5]) and prop-2-enoic acid (9.09 mg, 126 μmol) were added and the mixture stirred for 25 min at rt. Water (0.1 ml) was added and the mixture purified by reverse phase preparative HPLC (acidic), yielded the title compound 23.0 mg (98% purity, 61% yield).

LC-MS (Method 1): Rₜ=0.80 min; MS (ESIpos): m/z=440.2 [M+H]⁺

¹H-NMR (400 MHz, DMSO-d6) δ [ppm]: 0.910 (0.53), 0.922 (1.03), 0.927 (1.53), 0.937 (1.61), 0.940 (1.89), 0.946 (1.57), 0.954 (1.35), 0.962 (1.20), 1.462 (0.66), 1.484 (1.01), 1.493 (0.94), 1.533 (0.58), 2.331 (1.16), 2.336 (0.52), 2.518 (6.86), 2.523 (4.52), 2.673 (1.14), 2.678 (0.53), 4.099 (0.99), 4.168 (2.64), 4.277 (2.78), 4.830 (4.21), 4.929 (1.10), 5.775 (1.37), 5.803 (1.22), 6.169 (1.69), 6.210 (1.86), 6.540 (1.71), 6.587 (0.63), 6.626 (4.55), 6.856 (2.69), 6.874 (5.48), 6.893 (3.23), 6.930 (0.83), 6.958 (0.85), 6.972 (0.77), 6.998 (0.63), 7.200 (10.33), 7.222 (16.00), 7.239 (7.68), 7.245 (7.32), 7.425 (3.67), 7.439 (4.56), 7.460 (2.82), 7.561 (7.30), 7.581 (6.50), 8.125 (4.15), 8.134 (2.16), 8.139 (4.04), 9.012 (0.59), 9.062 (1.90).

### Example 21

### (2E)-1-[3-(2-anilinopyridin-4-yl)-2-(4-fluorophenyl)-6,7-dihydropyrazolo[1,5-a]pyrazin-5(4H)-yl]-4-(dimethylamino)but-2-en-1-one

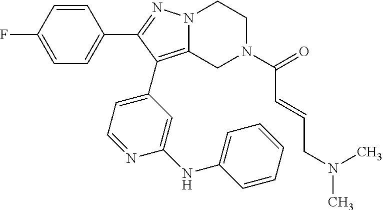 [SMILES: CN(C)C/C=C/C(=O)N1CCn2nc(-c3ccc(F)cc3)c(-c3ccnc(Nc4ccccc4)c3)c2C1]

Trifluoroacetic acid-4-[2-(4-fluorophenyl)-4,5,6,7-tetrahydropyrazolo[1,5-a]pyrazin-3-yl]-N-phenylpyridin-2-amine (1/1) (42.0 mg, 84.1 μmol, Intermediate 34) was dissolved in 1 ml DMF, then 2,4,6-Tripropyl-1,3,5,2,4,6-trioxatriphosphorinane-2,4,6-trioxide solution in DMF (80.3 mg, 50% purity, 126 μmol), N,N-diisopropylethylamine (88 μl, 500 μmol; CAS-RN: [7087-68-5]) and (2E)-4-(dimethylamino)but-2-enoic acid (16.3 mg, 126 μmol; CAS-RN: [98548-82-4]) were added and the mixture stirred for 25 min at rt. Water (0.1 ml) was added and the mixture purified by reverse phase preparative HPLC (basic), yielded the title compound 22.0 mg (98% purity, 52% yield).

LC-MS (Method 1): Rₜ=0.60 min; MS (ESIpos): m/z=497.2 [M+H]⁺

¹H-NMR (400 MHz, DMSO-d6) δ [ppm]: 2.084 (3.50), 2.090 (3.53), 2.155 (14.75), 2.327 (1.95), 2.331 (1.41), 2.336 (0.66), 2.518 (9.20), 2.523 (5.92), 2.669 (1.91), 2.673 (1.36), 2.678 (0.62), 3.048 (3.54), 3.062 (3.13), 4.148 (2.84), 4.268 (3.04), 4.812 (4.00), 4.907 (0.97), 6.534 (1.81), 6.618 (5.09), 6.694 (1.53), 6.748 (1.60), 6.785 (0.69), 6.849 (3.04), 6.867 (6.44), 6.886 (3.67), 7.195 (8.12), 7.198 (9.37), 7.203 (3.63), 7.216 (13.00), 7.221 (16.00), 7.235 (7.69), 7.243 (7.99), 7.422 (4.70), 7.436 (5.73), 7.443 (5.14), 7.458 (3.76), 7.562 (7.45), 7.582 (6.51), 8.127 (6.90), 8.141 (6.41), 8.993 (0.76), 9.036 (2.44).

### Example 22

### 1-[2-(4-fluorophenyl)-3-(1H-pyrrolo[2,3-b]pyridin-4-yl)-6,7-dihydropyrazolo[1,5-a]pyrazin-5(4H)-yl]prop-2-en-1-one

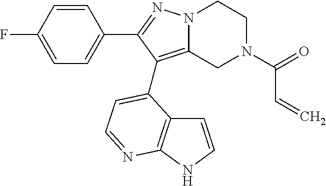 [SMILES: C=CC(=O)N1CCn2nc(-c3ccc(F)cc3)c(-c3ccnc4[nH]ccc34)c2C1]

Trifluoroacetic acid-2-(4-fluorophenyl)-3-(1H-pyrrolo[2,3-b]pyridin-4-yl)-4,5,6,7-tetrahydropyrazolo[1,5-a]pyrazine (1/1) (65.0 mg, 145 μmol, Intermediate 36) was dissolved in 1.8 ml DMF, then 2,4,6-Tripropyl-1,3,5,2,4,6-trioxatriphosphorinane-2,4,6-trioxide solution in DMF (130 μl, 50% purity, 220 μmol), N,N-diisopropylethylamine (150 μl, 870 μmol; CAS-RN: [7087-68-5]) and prop-2-enoic acid (15.7 mg, 218 μmol) were added and the mixture stirred for 25 min at rt. Water (0.1 ml) was added and the mixture purified by reverse phase preparative HPLC (acidic), yielded the title compound 31 mg (98% purity, 54% yield).

LC-MS (Method 1): Rₜ=0.77 min; MS (ESIpos): m/z=388.2 [M+H]⁺

¹H-NMR (400 MHz, DMSO-d6) δ [ppm]: 2.336 (0.53), 2.518 (6.93), 2.523 (4.72), 2.673 (1.17), 2.678 (0.53), 4.111 (1.45), 4.184 (4.04), 4.286 (1.69), 4.323 (4.65), 4.677 (7.29), 4.783 (1.61), 5.672 (0.58), 5.756 (1.99), 5.783 (1.98), 5.971 (4.16), 6.090 (0.49), 6.138 (2.30), 6.180 (2.14), 6.687 (0.42), 6.877 (3.01), 6.888 (3.26), 6.915 (1.80), 6.941 (2.20), 6.956 (1.99), 6.968 (0.89), 6.983 (1.15), 7.042 (4.66), 7.064 (9.48), 7.086 (5.19), 7.100 (0.66), 7.308 (4.91), 7.322 (5.62), 7.341 (3.26), 7.355 (1.20), 7.382 (4.48), 8.215 (16.00), 8.227 (15.30), 8.243 (0.45), 11.725 (3.11).

### Example 23

### (2E)-1-[2-(4-fluorophenyl)-3-(pyridin-4-yl)-6,7-dihydropyrazolo[1,5-a]pyrazin-5(4H)-yl]but-2-en-1-one

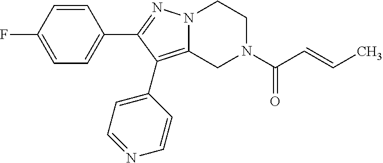 [SMILES: C/C=C/C(=O)N1CCn2nc(-c3ccc(F)cc3)c(-c3ccncc3)c2C1]

Using an analogous method as described for example 1, trifluoroacetic acid-2-(4-fluorophenyl)-3-(pyridin-4-yl)-4,5,6,7-tetrahydropyrazolo[1,5-a]pyrazine (1/1) (85.0 mg, 50% purity, 104 μmol, Intermediate 4) and (2E)-but-2-enoic acid (13.4 mg, 156 μmol) as the starting materials, 5.40 mg (90% purity, 13% yield) were prepared after purification by preparative HPLC (acidic conditions).

LC-MS (Method 1): Rₜ=0.62 min; MS (ESIpos): m/z=363.2 [M+H]⁺1H-NMR (400 MHz, DMSO-d6) δ [ppm]: 0.908 (0.97), 0.926 (3.02), 0.944 (3.31), 0.962 (1.96), 0.977 (0.63), 1.239 (0.74), 1.255 (0.64), 1.465 (0.75), 1.484 (1.08), 1.492 (0.87), 1.515 (0.88), 1.531 (0.90), 1.560 (0.56), 1.575 (0.52), 1.603 (0.54), 1.619 (0.74), 1.641 (0.69), 1.857 (3.17), 2.332 (2.34), 2.518 (16.00), 2.522 (9.65), 2.673 (2.36), 4.140 (1.22), 4.254 (d1.60), 4.818 (1.33), 6.685 (0.56), 6.719 (0.61), 6.735 (1.95), 6.751 (1.84), 6.772 (1.01), 6.789 (0.99), 6.947 (0.65), 7.075 (0.70), 7.175 (5.43), 7.197 (7.00), 7.220 (3.66), 7.352 (1.97), 7.366 (2.52), 7.386 (1.51), 8.545 (2.96), 8.557 (2.85).

### Example 24

### 1-[2-(4-fluorophenyl)-3-(pyridin-4-yl)-6,7-dihydropyrazolo[1,5-a]pyrazin-5(4H)-yl]-2-methylprop-2-en-1-one

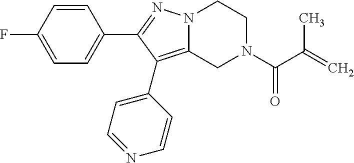 [SMILES: C=C(C)C(=O)N1CCn2nc(-c3ccc(F)cc3)c(-c3ccncc3)c2C1]

Using an analogous method as described for example 1, trifluoroacetic acid-2-(4-fluorophenyl)-3-(pyridin-4-yl)-4,5,6,7-tetrahydropyrazolo[1,5-a]pyrazine (1/1) (85.0 mg, 50% purity, 104 μmol, Intermediate 4) and 2-methylprop-2-enoic acid (13.4 mg, 156 μmol) as the starting materials, 6.00 mg (90% purity, 14% yield) were prepared after purification by preparative HPLC (acidic conditions).

LC-MS (Method 1): Rₜ=0.62 min; MS (ESIpos): m/z=363.2 [M+H]⁺

¹H-NMR (400 MHz, DMSO-d6) δ [ppm]: 0.908 (1.56), 0.927 (4.11), 0.930 (4.20), 0.939 (3.09), 0.952 (5.42), 0.958 (2.76), 0.963 (2.50), 0.970 (2.72), 1.041 (0.73), 1.049 (0.54), 1.059 (0.88), 1.066 (0.61), 1.088 (0.70), 1.106 (0.83), 1.467 (1.33), 1.484 (1.97), 1.492 (1.52), 1.504 (1.45), 1.516 (1.58), 1.533 (1.74), 1.565 (1.02), 1.580 (0.94), 1.600 (0.76), 1.608 (0.76), 1.625 (0.98), 1.645 (0.91), 1.661 (0.74), 1.681 (0.87), 1.702 (0.96), 1.723 (0.72), 1.776 (0.49), 1.796 (0.55), 1.822 (0.63), 1.838 (1.27), 1.892 (12.16), 2.323 (3.11), 2.327 (4.16), 2.332 (3.12), 2.518 (15.22), 2.522 (8.34), 2.665 (2.42), 2.669 (3.48), 2.673 (2.43), 3.663 (1.05), 3.692 (0.89), 4.035 (4.23), 4.048 (7.67), 4.062 (5.09), 4.275 (5.32), 4.817 (8.79), 5.164 (2.62), 5.299 (5.59), 6.946 (0.48), 7.074 (0.51), 7.181 (13.57), 7.196 (9.31), 7.203 (16.00), 7.225 (7.98), 7.359 (6.95), 7.373 (7.91), 7.381 (6.65), 7.395 (5.36), 8.559 (6.51), 8.571 (6.26).

### Example 25

### (2E)-1-[2-(4-chloro-3-fluorophenyl)-3-(pyridin-4-yl)-6,7-dihydropyrazolo[1,5-a]pyrazin-5(4H)-yl]-4-(dimethylamino)but-2-en-1-one

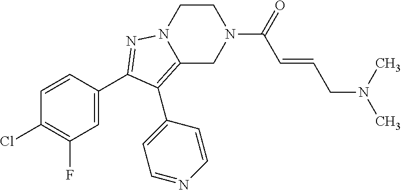 [SMILES: CN(C)C/C=C/C(=O)N1CCn2nc(-c3ccc(Cl)c(F)c3)c(-c3ccncc3)c2C1]

To a solution of 2-(4-chloro-3-fluorophenyl)-3-(pyridin-4-yl)-4,5,6,7-tetrahydropyrazolo[1,5-a]pyrazine (100 mg, 0.273 mmol, Intermediate 46) and (2E)-4-(dimethylamino)but-2-enoic acid hydrochloride (1:1) (68.0 mg, 0.410 mmol) in N,N-dimethylformamide (9.0 ml) were added 1-[bis(dimethylamino)methylene]-1H-1,2,3-triazolo[4,5-b]pyridinium-3-oxide hexafluorophosphate (156 mg, 0.410 mmol) and N,N-diisopropylethylamine (106 mg, 0.821 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 30%-60%, 9 min) to give (2E)-1-[2-(4-chloro-3-fluorophenyl)-3-(pyridin-4-yl)-6,7-dihydropyrazolo[1,5-a]pyrazin-5(4H)-yl]-4-(dimethylamino)but-2-en-1-one (33.5 mg, 0.0706 mmol, 25% yield) as a white solid.

LC-MS (Method 11): Rₜ=0.431 min; MS (ESIpos): m/z=439.1 [M+H]⁺.

¹H NMR (400 MHz, DMSO-d₆) δ=8.58 (d, J=5.6 Hz, 2H), 7.57 (t, J=8.4 Hz, 1H), 7.34 (d, J=9.6 Hz, 1H), 7.24-7.12 (m, 3H), 6.78-6.60 (m, 2H), 5.00-4.76 (m, 2H), 4.29 (br s, 2H), 4.14 (br s, 2H), 3.04 (br s, 2H), 2.22-2.08 (m, 6H).

### Example 26

### (2E)-1-[2-(4-chloro-2-fluorophenyl)-3-(pyridin-4-yl)-6,7-dihydropyrazolo[1,5-a]pyrazin-5(4H)-yl]-4-(dimethylamino)but-2-en-1-one

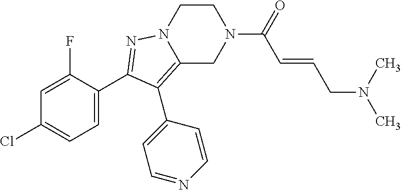 [SMILES: CN(C)C/C=C/C(=O)N1CCn2nc(-c3ccc(Cl)cc3F)c(-c3ccncc3)c2C1]

To a solution of (2E)-4-(dimethylamino)but-2-enoic acid (35.4 mg, 274 μmol) in dichloromethane (5 ml) were added N,N-diisopropylethylamine (190 μl, 1.1 mmol) and 1-[bis(dimethylamino)methylene]-1H-1,2,3-triazolo[4,5-b]pyridinium 3-oxid hexafluorophosphate (156 mg, 411 μmol) at 20° C. After stirring at 20° C. for 0.1 hour, to this solution was added 2-(4-chloro-2-fluorophenyl)-3-(pyridin-4-yl)-4,5,6,7-tetrahydropyrazolo[1,5-a]pyrazine hydrogen chloride (100 mg, 274 μmol, Intermediate 52) and the resulting mixture was stirred at 20° C. for 2 hours. The reaction mixture was concentrated under vacuum to give a crude product and the crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 31-61% B; flow 25 ml/min; temperature: RT; Detector: UV 220/254 nm] to give (2E)-1-[2-(4-chloro-2-fluorophenyl)-3-(pyridin-4-yl)-6,7-dihydropyrazolo[1,5-a]pyrazin-5(4H)-yl]-4-(dimethylamino)but-2-en-1-one (5.00 mg, 10.6 μmol, 93% purity, 4% yield) as a brown solid.

LC-MS (Method 12): Rₜ=0.908 min; MS (ESIpos): m/z=440.1 [M+H]⁺.

¹H NMR (400 MHz, DMSO-d₆) δ [ppm]=8.51 (d, J=4.4 Hz, 2H), 7.59-7.32 (m, 3H), 7.08 (s, 2H), 6.83-6.52 (m, 2H), 5.14-4.77 (m, 2H), 4.38-4.06 (m, 4H), 3.12-3.00 (m, 2H), 2.23-1.99 (m, 6H).

### Example 27

### (2E)-1-[2-(3-chloro-4-methoxyphenyl)-3-(pyridin-4-yl)-6,7-dihydropyrazolo[1,5-a]pyrazin-5(4H)-yl]-4-(dimethylamino)but-2-en-1-one

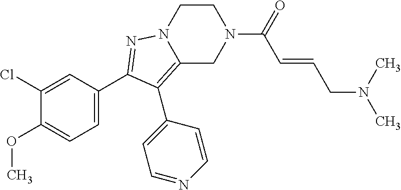 [SMILES: COc1ccc(-c2nn3c(c2-c2ccncc2)CN(C(=O)/C=C/CN(C)C)CC3)cc1Cl]

To a solution of 2-(3-chloro-4-methoxyphenyl)-3-(pyridin-4-yl)-4,5,6,7-tetrahydropyrazolo[1,5-a]pyrazine hydrogen chloride (100 mg, 0.265 mmol, Intermediate 58) and (2E)-4-(dimethylamino)but-2-enoic acid hydrogen chloride (1/1) (65.8 mg, 0.397 mmol) in N,N-dimethylformamide (9.0 ml) were added 1-[bis(dimethylamino)methylene]-1H-1,2,3-triazolo[4,5-b]pyridinium-3-oxide hexafluorophosphate (151 mg, 0.397 mmol) and N,N-diisopropylethylamine (102 mg, 0.795 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 25%-55%, 9 min) to give (2E)-1-[2-(3-chloro-4-methoxyphenyl)-3-(pyridin-4-yl)-6,7-dihydropyrazolo[1,5-a]pyrazin-5(4H)-yl]-4-(dimethylamino)but-2-en-1-one (27.5 mg, 0.0570 mmol, 21% yield) as a white solid.

LC-MS (Method 11): Rₜ=0.414 min; MS (ESIpos): m/z=451.1 [M+H]⁺.

¹H NMR (400 MHz, DMSO-d6) δ=8.57 (d, J=5.6 Hz, 2H), 7.39 (br s, 1H), 7.22-7.11 (m, 4H), 6.85-6.57 (m, 2H), 4.98-4.73 (m, 2H), 4.34-4.20 (m, 2H), 4.18-4.06 (m, 2H), 3.93-3.80 (m, 4H), 3.17-2.99 (m, 2H), 2.23-2.08 (m, 7H).

### Example 28

### (2E)-4-(dimethylamino)-1-[2-(2-methoxyphenyl)-3-(pyridin-4-yl)-6,7-dihydropyrazolo[1,5-a]pyrazin-5(4H)-yl]but-2-en-1-one

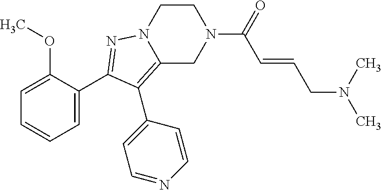 [SMILES: COc1ccccc1-c1nn2c(c1-c1ccncc1)CN(C(=O)/C=C/CN(C)C)CC2]

To a solution of (2E)-4-(dimethylamino)but-2-enoic acid (41.4 mg, 321 μmol) in dichloromethane (5 ml) were added N,N-diisopropylethylamine (220 μl, 1.3 mmol) and 1-[Bis(dimethylamino)methylene]-1H-1,2,3-triazolo[4,5-b]pyridinium 3-oxid hexafluorophosphate (183 mg, 481 μmol) at 20° C. After stirring at 20° C. for 0.1 hour, to this solution was added 2-(2-methoxyphenyl)-3-(pyridin-4-yl)-4,5,6,7-tetrahydropyrazolo[1,5-a]pyrazine hydrogen chloride (110 mg, 321 μmol, Intermediate 64) and the resulting mixture was stirred at 20° C. for 2 hours. The reaction mixture was concentrated under vacuum to give a crude product and the crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-32 min 0-50% B; flow 25 ml/min; temperature: RT; Detector: UV 220/254 nm] to give (2E)-4-(dimethylamino)-1-[2-(2-methoxyphenyl)-3-(pyridin-4-yl)-6,7-dihydropyrazolo[1,5-a]pyrazin-5(4H)-yl]but-2-en-1-one (5.00 mg, 11.6 μmol, 97% purity, 4% yield) as a white solid.

LC-MS (Method 12): Rₜ=0.852 min; MS (ESIpos): m/z=418.1 [M+H]⁺.

¹H NMR (400 MHz, DMSO-d6) δ [ppm]=8.43 (d, J=4.8 Hz, 2H), 7.48-7.26 (m, 2H), 7.10-6.92 (m, 4H), 6.84-6.61 (m, 2H), 5.13-4.81 (m, 2H), 4.38-3.94 (m, 4H), 3.27 (s, 3H), 3.15-2.94 (m, 2H), 2.25-2.07 (m, 6H).

### Example 29

### (2E)-1-[2-(3,4-dichlorophenyl)-3-(pyridin-4-yl)-6,7-dihydropyrazolo[1,5-a]pyrazin-5(4H)-yl]-4-(dimethylamino)but-2-en-1-one

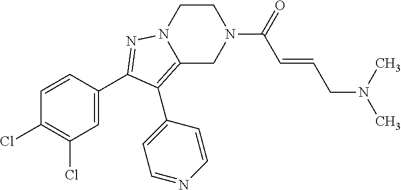 [SMILES: CN(C)C/C=C/C(=O)N1CCn2nc(-c3ccc(Cl)c(Cl)c3)c(-c3ccncc3)c2C1]

To a solution of 2-(3,4-dichlorophenyl)-3-(pyridin-4-yl)-4,5,6,7-tetrahydropyrazolo[1,5-a]pyrazine (70.0 mg, 0.202 mmol, Intermediate 70) and 2E)-4-(dimethylamino)but-2-enoic acid (39.2 mg, 0.304 mmol) in N,N-dimethylformamide (13 ml) were added 1-[bis(dimethylamino)methylene]-1H-1,2,3-triazolo[4,5-b]pyridinium-3-oxide hexafluorophosphate (115 mg, 0.304 mmol) and N,N-diisopropylethylamine (78.6 mg, 0.608 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Unisil 3-100 C18 Ultra 150*50 mm*3 μm; mobile phase: water (FA)-ACN; B %: 0%-30%, 7 min) to give (2E)-1-[2-(3,4-dichlorophenyl)-3-(pyridin-4-yl)-6,7-dihydropyrazolo[1,5-a]pyrazin-5(4H)-yl]-4-(dimethylamino)but-2-en-1-one (19.1 mg, 0.0383 mmol, 19% yield) as a white solid.

LC-MS (Method 11): Rₜ=0.445 min; MS (ESIpos): m/z=455.1 [M+H]⁺.

¹H NMR (400 MHz, DMSO-d6) δ=8.58 (d, J=4.8 Hz, 2H), 8.19 (s, 1H), 7.68-7.49 (m, 2H), 7.33-7.06 (m, 3H), 6.81-6.60 (m, 1H), 6.55-6.53 (m, 1H), 6.84-6.46 (m, 1H), 5.04-4.66 (m, 2H), 4.38-4.18 (m, 2H), 4.16-3.97 (m, 2H), 3.11-2.99 (m, 2H), 2.22-2.00 (m, 1H), 2.20-1.99 (m, 5H).

### Example 30

### (2E)-4-(dimethylamino)-1-[2-(4-methoxyphenyl)-3-(pyridin-4-yl)-6,7-dihydropyrazolo[1,5-a]pyrazin-5(4H)-yl]but-2-en-1-one

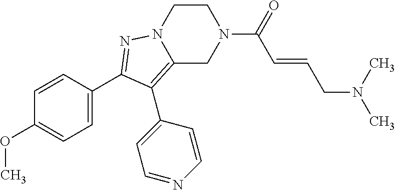 [SMILES: COc1ccc(-c2nn3c(c2-c2ccncc2)CN(C(=O)/C=C/CN(C)C)CC3)cc1]

To a solution of 2-(4-methoxyphenyl)-3-(pyridin-4-yl)-4,5,6,7-tetrahydropyrazolo[1,5-a]pyrazine hydrogen chloride (130 mg, 0.379 mmol, Intermediate 76) and (2E)-4-(dimethylamino)but-2-enoic acid-hydrogen chloride (1/1) (94.2 mg, 0.568 mmol) in N,N-dimethylformamide (5.8 ml) were added 1-[bis(dimethylamino)methylene]-1H-1,2,3-triazolo[4,5-b]pyridinium-3-oxide hexafluorophosphate (216 mg, 0.568 mmol) and N,N-diisopropylethylamine (98.0 mg, 0.758 mmol) at 25° C. The mixture was stirred at 25° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Unisil 3-100 C18 Ultra 150*50 mm*3 μm; mobile phase: water (FA)-ACN; B %: 1%-19%, 7 min) to give (2E)-4-(dimethylamino)-1-[2-(4-methoxyphenyl)-3-(pyridin-4-yl)-6,7-dihydropyrazolo[1,5-a]pyrazin-5(4H)-yl]but-2-en-1-one (13.1 mg, 0.0275 mmol, 7% yield) as a white solid.

LC-MS (Method 11): Rₜ=0.438 min; MS (ESIpos): m/z=471.2 [M+H]⁺.

¹H NMR (400 MHz, DMSO-d6) δ [ppm]=8.53 (d, J=4.8 Hz, 2H), 8.20 (s, 1H), 7.27 (d, J=7.6 Hz, 2H), 7.16 (d, J=3.6 Hz, 2H), 6.96-6.85 (m, 2H), 6.80-6.58 (m, 2H), 4.95-4.79 (m, 2H), 4.25 (br s, 2H), 4.17-4.07 (m, 2H), 3.78-3.74 (m, 3H), 3.06 (br s, 2H), 2.29-2.01 (m, 6H).

### Example 31

### (2E)-4-(dimethylamino)-1-[2-(3-fluoro-4-methoxyphenyl)-3-(pyridin-4-yl)-6,7-dihydropyrazolo[1,5-a]pyrazin-5(4H)-yl]but-2-en-1-one

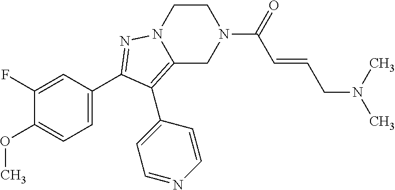 [SMILES: COc1ccc(-c2nn3c(c2-c2ccncc2)CN(C(=O)/C=C/CN(C)C)CC3)cc1F]

To a solution of 2-(3-fluoro-4-methoxyphenyl)-3-(pyridin-4-yl)-4,5,6,7-tetrahydropyrazolo[1,5-a]pyrazine hydrogen chloride (140 mg, 0.388 mmol, Intermediate 82) and (2E)-4-(dimethylamino)but-2-enoic acid·hydrogen chloride (1/1) (96.3 mg, 0.582 mmol) in N,N-dimethylformamide (6.2 ml) were added 1-[bis(dimethylamino)methylene]-1H-1,2,3-triazolo[4,5-b]pyridinium-3-oxide hexafluorophosphate (221 mg, 0.582 mmol) and N,N-diisopropylethylamine (100 mg, 0.776 mmol) at 25° C. The mixture was stirred at 25° C. for 16 hours. The reaction solution was poured into water, extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge C18 150*50 mm*10 μm; mobile phase: water (NH4HCO3)-ACN; B %: 22%-52%, 10 min) to give (2E)-4-(dimethylamino)-1-[2-(3-fluoro-4-methoxyphenyl)-3-(pyridin-4-yl)-6,7-dihydropyrazolo[1,5-a]pyrazin-5(4H)-yl]but-2-en-1-one (8.4 mg, 0.0173 mmol, 90% purity, 4% yield) as a white solid.

LC-MS (Method 11): Rₜ=0.447 min; MS (ESIpos): m/z=435.2 [M+H]⁺

¹H NMR (400 MHz, DMSO-d6) δ [ppm]=8.56 (d, J=5.2 Hz, 2H), 7.22-7.16 (m, 2H), 7.13 (d, J=8.8 Hz, 1H), 7.10-7.03 (m, 1H), 6.82-6.57 (m, 2H), 4.98-4.65 (m, 2H), 4.26 (br s, 2H), 4.18-4.02 (m, 2H), 3.83 (s, 3H), 3.05 (d, J=5.2 Hz, 2H), 2.27-1.95 (m, 6H).

### Example 32

### formic acid-(2E)-1-[2-(4-chloro-3-ethylphenyl)-3-(pyridin-4-yl)-6,7-dihydropyrazolo[1,5-a]pyrazin-5(4H)-yl]-4-(dimethylamino)but-2-en-1-one (1:1)

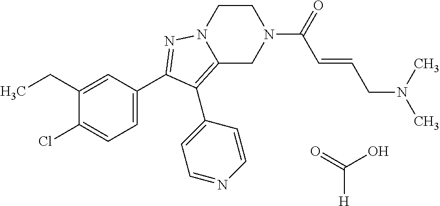 [SMILES: CCc1cc(-c2nn3c(c2-c2ccncc2)CN(C(=O)/C=C/CN(C)C)CC3)ccc1Cl ; O=CO]

To a solution of 2-(4-chloro-3-ethylphenyl)-3-(pyridin-4-yl)-4,5,6,7-tetrahydropyrazolo[1,5-a]pyrazine (120 mg, 94% purity, Intermediate 292) in N,N-dimethylformamide (2.0 ml) were added (2E)-4-(dimethylamino)but-2-enoic acid (47.3 mg, 366 μmol), O-(7-azabenzotriazol-1-yl)-N,N,N′,N′-tetramethyluronium hexafluorophosphate (190 mg, 499 μmol) and N,N-diisopropylethylamine (230 μl, 1.30 mmol) at 25° C. The reaction mixture was stirred at 25° C. for 16 hours. The reaction mixture was poured into water and extracted with ethyl acetate. The organic phase was washed with brine, dried over anhydrous sodium sulfate, filtered and concentrated in vacuo to give a residue. The residue was purified by preparative-HPLC (Instrument: Gilson-281; Column: Phenomenex C18 75*30 mm*3 μm; eluent A: 0.1% formic acid in water, eluent B: acetonitrile; gradient: 0-9 min 5-35% B; flow 25 ml/min; temperature: room temperature; Detector: UV 220/254 nm) to give formic acid-(2E)-1-[2-(4-chloro-3-ethylphenyl)-3-(pyridin-4-yl)-6,7-dihydropyrazolo[1,5-a]pyrazin-5(4H)-yl]-4-(dimethylamino)but-2-en-1-one (1:1) (25.0 mg, 95% purity, 14% yield) as a pale yellow solid.

LC-MS (Method 13): Rₜ=0.676 min; MS (ESIpos): m/z=450.1 [M+H]⁺.

¹H NMR (400 MHz, DMSO-d₆) δ [ppm]=8.56 (d, J=3.6 Hz, 2H), 7.44 (d, J=6.8 Hz, 1H), 7.35 (s, 1H), 7.18 (s, 3H), 6.93-6.64 (m, 2H), 5.07-4.66 (m, H), 4.35-4.22 (m, 2H), 4.18-4.06 (m, 2H), 3.29 (s, 1H), 2.63 (t, J=7.6 Hz, 2H), 2.42-2.23 (m, 6H), 1.05 (t, J=7.6 Hz, 3H).

### Example 33

### 4-{5-[(2E)-4-(dimethylamino)but-2-enoyl]-3-(pyridin-4-yl)-4,5,6,7-tetrahydropyrazolo[1,5-a]pyrazin-2-yl}benzonitrile

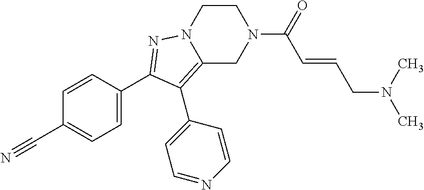 [SMILES: CN(C)C/C=C/C(=O)N1CCn2nc(-c3ccc(C#N)cc3)c(-c3ccncc3)c2C1]

To a solution of 4-[3-(pyridin-4-yl)-4,5,6,7-tetrahydropyrazolo[1,5-a]pyrazin-2-yl]benzonitrile hydrochloride (110 mg, 75% purity, Intermediate 84) in N,N-dimethylformamide (2 ml) were added (2E)-4-(dimethylamino)but-2-enoic acid (38.9 mg, 301 μmol), O-(7-azabenzotriazol-1-yl)-N,N,N′,N′-tetramethyluronium hexafluorophosphate (156 mg, 411 μmol) and N,N-diisopropylethylamine (190 μl, 1.1 mmol) at 25° C. The reaction mixture was stirred at 25° C. for 16 hours. The reaction mixture was poured into water and extracted with ethyl acetate. The organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 23-53% B; flow 25 ml/min; temperature: room temperature; Detector: UV 220/254 nm) to give 4-{5-[(2E)-4-(dimethylamino)but-2-enoyl]-3-(pyridin-4-yl)-4,5,6,7-tetrahydropyrazolo[1,5-a]pyrazin-2-yl}benzonitrile (6.50 mg, 97% purity, 6% yield) as a white solid.

LC-MS (Method 11): Rₜ=1.009 min; MS (ESIpos): m/z=413.2 [M+H]⁺.

¹H NMR (400 MHz, DMSO-d₆) δ [ppm]=8.58 (d, J=4.8 Hz, 2H), 7.82 (d, J=8.4 Hz, 2H), 7.53 (d, J=16.0 Hz, 2H), 7.20 (d, J=4.4 Hz, 2H), 6.84-6.50 (m, 2H), 5.07-4.66 (m, 2H), 4.40-4.22 (m, 2H), 4.19-4.06 (m, 2H), 3.14-2.99 (m, 2H), 2.21-2.03 (m, 6H).

### Example 34

### (2E)-4-(dimethylamino)-1-[3-(pyridin-4-yl)-2-[4-(trifluoromethyl)phenyl]-6,7-dihydropyrazolo[1,5-a]pyrazin-5(4H)-yl]but-2-en-1-one

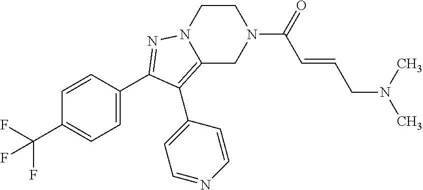 [SMILES: CN(C)C/C=C/C(=O)N1CCn2nc(-c3ccc(C(F)(F)F)cc3)c(-c3ccncc3)c2C1]

To a solution of (2E)-4-(dimethylamino)but-2-enoic acid hydrogen chloride (52.2 mg, 315 μmol) in dichloromethane (10 ml) were added N,N-diisopropylethylamine (150 μl, 840 μmol) and 1-[Bis(dimethylamino)methylene]-1H-1,2,3-triazolo[4,5-b]pyridinium 3-oxid hexafluorophosphate (120 mg, 315 μmol) at 20° C. The mixture was stirred at 20° C. for 10 minutes. Then 3-(pyridin-4-yl)-2-[4-(trifluoromethyl)phenyl]-4,5,6,7-tetrahydropyrazolo[1,5-a]pyrazine hydrogen chloride (80.0 mg, 210 μmol, Intermediate 90) was added and the resulting mixture was stirred at 20° C. for 2 hours. The reaction mixture was concentrated under vacuum to give a crude product and the crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 32-62% B; flow 25 ml/min; temperature: RT; Detector: UV 220/254 nm] to give (2E)-4-(dimethylamino)-1-[3-(pyridin-4-yl)-2-[4-(trifluoromethyl)phenyl]-6,7-dihydropyrazolo[1,5-a]pyrazin-5(4H)-yl]but-2-en-1-one (29.5 mg, 62.8 μmol, 97% purity, 30% yield) as a brown solid.

LC-MS (Method 12): Rₜ=0.913 min; MS (ESIpos): m/z=456.1 [M+H]⁺.

¹H NMR (400 MHz, DMSO-d6) δ [ppm]=8.58 (d, J=5.6 Hz, 2H), 7.72 (d, J=8.4 Hz, 2H), 7.57 (d, J=7.6 Hz, 2H), 7.20 (d, J=5.2 Hz, 2H), 6.85-6.55 (m, 2H), 5.02-4.68 (m, 2H), 4.39-4.04 (m, 4H), 3.11-3.00 (m, 2H), 2.20-2.06 (m, 6H).

### Example 35

### (2E)-1-{2-[4-chloro-2-(hydroxymethyl)phenyl]-3-(pyridin-4-yl)-6,7-dihydropyrazolo[1,5-a]pyrazin-5(4H)-yl}-4-(dimethylamino)but-2-en-1-one

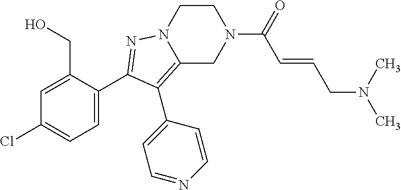 [SMILES: CN(C)C/C=C/C(=O)N1CCn2nc(-c3ccc(Cl)cc3CO)c(-c3ccncc3)c2C1]

To a solution of {5-chloro-2-[3-(pyridin-4-yl)-4,5,6,7-tetrahydropyrazolo[1,5-a]pyrazin-2-yl]phenyl}methanol (140 mg, 89% purity, Intermediate 92) in N,N-dimethylformamide (2.0 ml) were added (2E)-4-(dimethylamino)but-2-enoic acid (51.9 mg, 402 μmol), O-(7-azabenzotriazol-1-yl)-N,N,N′,N′-tetramethyluronium hexafluorophosphate (209 mg, 548 μmol) and N,N-diisopropylethylamine (190 μl, 1.1 mmol) at 25° C. The mixture was stirred at 25° C. for 16 hours. The reaction mixture was poured into water and extracted with ethyl acetate. The organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 20-50% B; flow 25 ml/min; temperature: room temperature; Detector: UV 220/254 nm) to give (2E)-1-{2-[4-chloro-2-(hydroxymethyl)phenyl]-3-(pyridin-4-yl)-6,7-dihydropyrazolo[1,5-a]pyrazin-5(4H)-yl}-4-(dimethylamino)but-2-en-1-one (58 mg, 96% purity, 34% yield) as a white solid.

LC-MS (Method 13): Rₜ=0.827 min; MS (ESIpos): m/z=452.0 [M+H]⁺.

¹H NMR (400 MHz, DMSO-d6) δ [ppm]=8.48 (d, J=5.2 Hz, 2H), 7.57 (d, J=2.0 Hz, 1H), 7.30 (dd, J=2.4 Hz, 2H), 7.09 (d, J=8.4 Hz, 1H), 7.04-6.98 (m, 2H), 6.81-6.64 (m, 2H), 5.22 (t, J=6.0 Hz, 1H), 5.10-4.88 (m, 2H), 4.34-4.27 (m, 3H), 4.26-4.03 (m, 3H), 3.10-2.98 (m, 2H), 2.22-2.07 (m, 6H).

### Example 36

### 1-[2-(4-chloro-3-fluorophenyl)-3-(pyridin-4-yl)-6,7-dihydropyrazolo[1,5-a]pyrazin-5(4H)-yl]prop-2-en-1-one

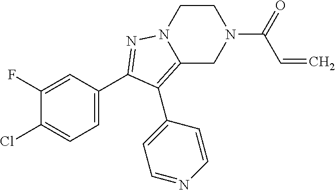 [SMILES: C=CC(=O)N1CCn2nc(-c3ccc(Cl)c(F)c3)c(-c3ccncc3)c2C1]

To a mixture of 2-(4-chloro-3-fluorophenyl)-3-(pyridin-4-yl)-4,5,6,7-tetrahydropyrazolo[1,5-a]pyrazine hydrochloride (1:1) (390 mg, 1.06 mmol, Intermediate 46) and acryloyl chloride (24.7 mg, 0.273 mmol) in dichloromethane (13.0 mL) was added triethylamine (0.1 ml, 0.821 mmol). The resulting mixture was stirred at 20° C. for 1 hour. The reaction mixture was concentrated in vacuo to give a residue. The residue was purified by preparative HPLC (column: Phenomenex C18 75*30 mm*3 μm; mobile phase: water (FA)-ACN; B %: 10%-40%, 7 min) to give 1-[2-(4-chloro-3-fluorophenyl)-3-(pyridin-4-yl)-6,7-dihydropyrazolo[1,5-a]pyrazin-5(4H)-yl]prop-2-en-1-one (17.4 mg, 0.0423 mmol, 15% yield) as a white solid.

LC-MS (Method 11): Rₜ=0.495 min; MS (ESIpos): m/z=382.1 [M+H]⁺.

¹H NMR (400 MHz, DMSO-d6) δ [ppm]=8.58 (d, J=4.4 Hz, 2H), 7.57 (t, J=8.4 Hz, 1H), 7.33 (d, J=10.4 Hz, 1H), 7.21 (br s, 3H), 7.15 (d, J=8.0 Hz, 1H), 7.03-6.82 (m, 1H), 6.17 (d, J=16.4 Hz, 1H), 5.78 (d, J=9.2 Hz, 1H), 4.95 (br s, 1H), 4.82 (br s, 2H), 4.29 (br s, 3H), 4.17-4.05 (m, 2H).

### Example 37

### 1-[2-(4-chloro-2-fluorophenyl)-3-(pyridin-4-yl)-6,7-dihydropyrazolo[1,5-a]pyrazin-5(4H)-yl]prop-2-en-1-one

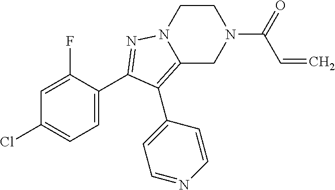 [SMILES: C=CC(=O)N1CCn2nc(-c3ccc(Cl)cc3F)c(-c3ccncc3)c2C1]

To a solution of 2-(4-chloro-2-fluorophenyl)-3-(pyridin-4-yl)-4,5,6,7-tetrahydropyrazolo[1,5-a]pyrazine hydrogen chloride (100 mg, 274 μmol, Intermediate 52) in dichloromethane (5 ml) were added triethylamine (110 μl, 820 μmol) and acryloyl chloride (24.8 mg, 274 μmol) at 20° C., then the mixture was stirred at 20° C. for 1 hour. The reaction mixture was concentrated under vacuum to give a crude product and the crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 27-55% B; flow 25 ml/min; temperature: RT; Detector: UV 220/254 nm] to give 1-[2-(4-chloro-2-fluorophenyl)-3-(pyridin-4-yl)-6,7-dihydropyrazolo[1,5-a]pyrazin-5(4H)-yl]prop-2-en-1-one (35.0 mg, 85.9 μmol, 94% purity, 31% yield) as a white solid.

LC-MS (Method 12): Rₜ=0.871 min; MS (ESIpos): m/z=383.0 [M+H]⁺.

¹H NMR (400 MHz, DMSO-d₆) δ [ppm]=8.51 (d, J=4.0 Hz, 2H), 7.63-7.30 (m, 3H), 7.08 (s, 2H), 7.02-6.80 (m, 1H), 6.19 (d, J=16.8 Hz, 1H), 5.79 (d, J=10.8 Hz, 1H), 5.15-4.84 (m, 2H), 4.40-4.00 (m, 4H)

### Example 38

### 1-[2-(3-chloro-4-methoxyphenyl)-3-(pyridin-4-yl)-6,7-dihydropyrazolo[1,5-a]pyrazin-5(4H)-yl]prop-2-en-1-one

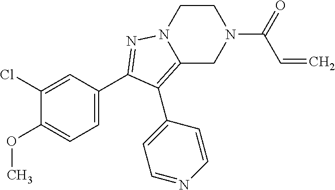 [SMILES: C=CC(=O)N1CCn2nc(-c3ccc(OC)c(Cl)c3)c(-c3ccncc3)c2C1]

To a mixture of 2-(3-chloro-4-methoxyphenyl)-3-(pyridin-4-yl)-4,5,6,7-tetrahydropyrazolo[1,5-a]pyrazine hydrochloride (1:1) (650 mg, 1.72 mmol, Intermediate 58) and acryloyl chloride (23.9 mg, 0.265 mmol) in dichloromethane (13.5 mL) was added triethylamine (0.1 ml, 0.795 mmol) at 20° C. The resulting mixture was stirred at 20° C. for 1 hour. The reaction mixture was concentrated in vacuo to give a residue. The residue was purified by preparative HPLC (column: Phenomenex C18 75*30 mm*3 μm; mobile phase: water (FA)-ACN; B %: 6%-36%, 7 min) to give 1-[2-(3-chloro-4-methoxyphenyl)-3-(pyridin-4-yl)-6,7-dihydropyrazolo[1,5-a]pyrazin-5(4H)-yl]prop-2-en-1-one (5.60 mg, 0.0139 mmol, 5% yield) as a white solid.

LC-MS (Method 11): Rₜ=0.486 min; MS (ESIpos): m/z=395.1 [M+H]⁺.

¹H NMR (400 MHz, DMSO-d₆) δ [ppm]=8.57 (d, J=3.6 Hz, 1H), 7.40 (br s, 1H), 7.28-7.18 (m, 3H), 7.17-7.11 (m, 1H), 7.03-6.80 (m, 1H), 6.18 (d, J=16.8 Hz, 1H), 5.84-5.68 (m, 1H), 5.01-4.76 (m, 2H), 4.37-4.20 (m, 2H), 4.19-4.04 (m, 2H), 3.86 (s, 3H).

### Example 39

### 1-[2-(2-methoxyphenyl)-3-(pyridin-4-yl)-6,7-dihydropyrazolo[1,5-a]pyrazin-5(4H)-yl]prop-2-en-1-one

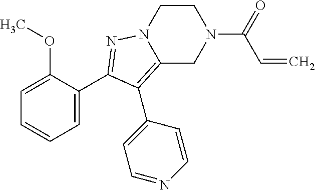 [SMILES: C=CC(=O)N1CCn2nc(-c3ccccc3OC)c(-c3ccncc3)c2C1]

To a solution of 2-(2-methoxyphenyl)-3-(pyridin-4-yl)-4,5,6,7-tetrahydropyrazolo[1,5-a]pyrazine hydrogen chloride (110 mg, 321 μmol, Intermediate 64) in dichloromethane (5 ml) were added N,N-Diisopropylethylamine (170 μl, 960 μmol) and acryloyl chloride (29.0 mg, 321 μmol) at 20° C., then the mixture was stirred at 20° C. for 1 hour. The reaction mixture was concentrated under vacuum to give a crude product and the crude product was purified by preparative HPLC [Instrument: ACSWH-GX-G; Column: Unisil 3-100 C18 Ultra 150*50 mm*3 μm, eluent A: water (0.2% formic acid), eluent B: acetonitrile; gradient: 0-7 min 1-29% B; flow 25 ml/min; temperature: RT; Detector: UV 220/254 nm] to give 1-[2-(2-methoxyphenyl)-3-(pyridin-4-yl)-6,7-dihydropyrazolo[1,5-a]pyrazin-5(4H)-yl]prop-2-en-1-one (32.3 mg, 87.8 μmol, 98% purity, 27% yield) as a white solid.

LC-MS (Method 12): Rₜ=0.819 min; MS (ESIpos): m/z=361.0 [M+H]⁺.

¹H NMR (400 MHz, DMSO-d₆) δ [ppm]=8.51 (d, J=4.4 Hz, 2H), 7.59-7.32 (m, 3H), 7.08 (s, 2H), 6.83-6.52 (m, 2H), 5.14-4.77 (m, 2H), 4.38-4.06 (m, 4H), 3.12-3.00 (m, 2H), 2.23-1.99 (m, 6H).

### Example 40

### 1-[2-(3,4-dichlorophenyl)-3-(pyridin-4-yl)-6,7-dihydropyrazolo[1,5-a]pyrazin-5(4H)-yl]prop-2-en-1-one

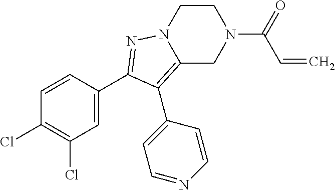 [SMILES: C=CC(=O)N1CCn2nc(-c3ccc(Cl)c(Cl)c3)c(-c3ccncc3)c2C1]

To a mixture of 2-(3,4-dichlorophenyl)-3-(pyridin-4-yl)-4,5,6,7-tetrahydropyrazolo[1,5-a]pyrazine (80.0 mg, 0.231 mmol, Intermediate 70) and acryloyl chloride (23.0 mg, 0.254 mmol) in dichloromethane (1.0 ml) was added triethylamine (1 ml, 7.17 mmol) at 20° C. The resulting mixture was stirred at 20° C. for 2 hours. The reaction mixture was concentrated in vacuo to give a residue. The residue was purified by preparative HPLC (column: Phenomenex luna C18 150*25 mm*10 μm; mobile phase: water (FA)-ACN; B %: 19%-49%, 10 min) to give 1-[2-(3-chloro-4-methoxyphenyl)-3-(pyridin-4-yl)-6,7-dihydropyrazolo[1,5-a]pyrazin-5(4H)-yl]prop-2-en-1-one (13.4 mg, 0.0324 mmol, 97% purity, 13% yield) as a white solid.

LC-MS (Method 11): Rₜ=0.523 min; MS (ESIpos): m/z=399.0 [M+H]⁺.

¹H NMR (400 MHz, DMSO-d₆) δ [ppm]=8.58 (br s, 2H), 7.72-7.44 (m, 2H), 7.36-7.14 (m, 3H), 6.92-6.91 (m, 1H), 7.0-6.74 (m, 1H), 6.24-6.11 (m, 1H), 5.86-5.62 (m, 1H), 5.04-4.66 (m, 2H), 4.34-4.05 (m, 4H).

### Example 41

### 1-[2-(4-methoxyphenyl)-3-(pyridin-4-yl)-6,7-dihydropyrazolo[1,5-a]pyrazin-5(4H)-yl]prop-2-en-1-one

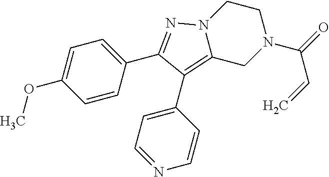 [SMILES: C=CC(=O)N1CCn2nc(-c3ccc(OC)cc3)c(-c3ccncc3)c2C1]

To a mixture of 2-(4-methoxyphenyl)-3-(pyridin-4-yl)-4,5,6,7-tetrahydropyrazolo[1,5-a]pyrazine hydrochloride (1:1) (120 mg, 0.350 mmol, Intermediate 76) and acryloyl chloride (63.4 mg, 0.700 mmol) in dichloromethane was added triethylamine (0.1 ml, 0.700 mmol) at 20° C. The resulting mixture was stirred at 20° C. for 1 hour. The reaction mixture was concentrated in vacuo to give a residue. The residue was purified by preparative HPLC (column: Waters Xbridge 150*25 mm*5 μm; mobile phase: water (NH4HCO3)-ACN; B %: 24%-54%, 8 min) to give 1-[2-(4-methoxyphenyl)-3-(pyridin-4-yl)-6,7-dihydropyrazolo[1,5-a]pyrazin-5(4H)-yl]prop-2-en-1-one (19.4 mg, 0.0520 mmol, 15% yield) as a gray solid.

LC-MS (Method 11): Rₜ=0.836 min; MS (ESIpos): m/z=361.0 [M+H]⁺.

¹H NMR (400 MHz, DMSO-da) δ [ppm]=8.53 (br s, 2H), 7.27 (d, J=8.0 Hz, 2H), 7.16 (d, J=8.0 Hz, 2H), 6.91 (d, J=8.8 Hz, 3H), 6.18 (d, J=16.8 Hz, 1H), 5.87-5.64 (m, 1H), 5.04-4.69 (m, 2H), 4.42-3.98 (m, 5H), 3.75 (s, 3H).

### Example 42

### 1-[2-(3-fluoro-4-methoxyphenyl)-3-(pyridin-4-yl)-6,7-dihydropyrazolo[1,5-a]pyrazin-5(4H)-yl]prop-2-en-1-one

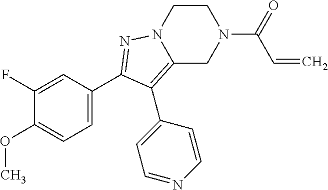 [SMILES: C=CC(=O)N1CCn2nc(-c3ccc(OC)c(F)c3)c(-c3ccncc3)c2C1]

To solution of 2-(3-fluoro-4-methoxyphenyl)-3-(pyridin-4-yl)-4,5,6,7-tetrahydropyrazolo[1,5-a]pyrazine hydrochloride (1:1) (60 mg, 0.166 mmol, Intermediate 82) and acryloyl chloride (45.2 mg, 0.499 mmol) in dichloromethane (4.5 ml) was added triethylamine (50.5 mg, 0.499 mmol) at 20° C. The mixture was stirred at 20° C. for 1 hour. The reaction mixture was poured into water and extracted with ethyl acetate. The combined organic layers were washed with brine, dried over anhydrous sodium sulfate, filtered and concentrated in vacuo to give a residue. The residue was purified by preparative HPLC (column: Waters Xbridge 150*25 mm*5 μm; mobile phase: water (ammonia hydroxide v/v)-ACN; B %: 22%-52%, 9 min) to give 1-[2-(3-fluoro-4-methoxyphenyl)-3-(pyridin-4-yl)-6,7-dihydropyrazolo[1,5-a]pyrazin-5(4H)-yl]prop-2-en-1-one (5.50 mg, 0.0143 mmol, 9% yield) as a pale yellow solid.

LC-MS (Method 11): Rₜ=0.837 min; MS (ESIpos): m/z=379.0 [M+H]⁺.

¹H NMR (400 MHz, DMSO-d6) δ [ppm]=8.56 (d, J=4.8 Hz, 2H), 7.29-7.02 (m, 5H), 7.01-6.79 (m, 1H), 6.17 (d, J=17.2 Hz, 1H), 5.95-5.58 (m, 1H), 5.00-4.68 (m, 2H), 4.35-3.97 (m, 4H), 3.84 (s, 3H).

### Example 43

### 4-[5-acryloyl-3-(pyridin-4-yl)-4,5,6,7-tetrahydropyrazolo[1,5-a]pyrazin-2-yl]benzonitrile

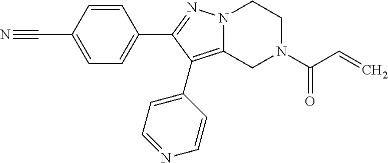 [SMILES: C=CC(=O)N1CCn2nc(-c3ccc(C#N)cc3)c(-c3ccncc3)c2C1]

To a solution of 4-[3-(pyridin-4-yl)-4,5,6,7-tetrahydropyrazolo[1,5-a]pyrazin-2-yl]benzonitrile hydrochloride (100 mg, 296 μmol, Intermediate 84) in dichloromethane (5 ml) were added N,N-diisopropylethylamine (115 mg, 888 μmol) and acryloyl chloride (26.8 mg, 296 μmol) at 25° C. and the mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 20-50% B; flow 25 ml/min; temperature: RT; Detector: UV 220/254 nm] to give 4-[5-acryloyl-3-(pyridin-4-yl)-4,5,6,7-tetrahydropyrazolo[1,5-a]pyrazin-2-yl]benzonitrile (46.2 mg, 127 μmol, 98% purity, 43% yield) as a white solid.

LC-MS (Method 12): Rₜ=0.787 min; MS (ESIpos): m/z=356.2 [M+H]⁺.

¹H NMR (400 MHz, DMSO-d₆) δ [ppm]=8.58 (d, J=4.4 Hz, 2H), 7.82 (d, J=8.4 Hz, 2H), 7.53 (d, J=7.6 Hz, 2H), 7.21 (s, 2H), 7.05-6.74 (m, 1H), 6.18 (d, J=17.2 Hz, 1H), 5.90-5.58 (m, 1H), 5.07-4.71 (m, 2H), 4.45-3.94 (m, 4H).

### Example 44

### 1-[3-(pyridin-4-yl)-2-[4-(trifluoromethyl)phenyl]-6,7-dihydropyrazolo[1,5-a]pyrazin-5(4H)-yl]prop-2-en-1-one

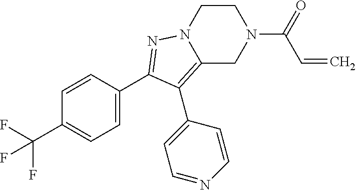 [SMILES: C=CC(=O)N1CCn2nc(-c3ccc(C(F)(F)F)cc3)c(-c3ccncc3)c2C1]

To a solution of 3-(pyridin-4-yl)-2-[4-(trifluoromethyl)phenyl]-4,5,6,7-tetrahydropyrazolo[1,5-a]pyrazine hydrochloride (1:1) (60.0 mg, 0.158 mmol, Intermediate 90) and acryloyl chloride (42.8 mg, 0.473 mmol) in dichloromethane (5 ml) was added triethylamine (0.06 ml) at 20° C. The mixture was stirred at 20° C. for 1 hour. The reaction mixture was poured into water and extracted with ethyl acetate. The combined organic layers were washed with brine, dried over anhydrous sodium sulfate, filtered and concentrated in vacuo to give a residue. The residue was purified by preparative HPLC (column: Phenomenex luna C18 150*25 mm*10 μm; mobile phase: water (FA)-ACN; B %: 10%-40%, 2 min) to give 1-[3-(pyridin-4-yl)-2-[4-(trifluoromethyl)phenyl]-6,7-dihydropyrazolo[1,5-a]pyrazin-5(4H)-yl]prop-2-en-1-one (3 mg, 0.00740 mmol, 5% yield) as a white solid.

LC-MS (Method 11): Rₜ=0.804 min; MS (ESIpos): m/z=399.2 [M+H]⁺.

¹H NMR (400 MHz, DMSO-d₆) δ [ppm]=8.58 (d, J=4.4 Hz, 2H), 7.87-7.67 (m, 3H), 7.58 (d, J=7.2 Hz, 2H), 7.21 (br s, 2H), 7.05-6.79 (m, 1H), 6.18 (d, J=16.4 Hz, 1H), 5.94-5.60 (m, 1H), 5.13-4.72 (m, 2H), 4.48-3.93 (m, 4H).

The following examples were prepared analogous to the preparation of example 3 starting from the corresponding intermediates by reacting with acrylic acid.

| Example | Structure | |
|---|---|---|
| No | Name | Analytical Data |
| 45 | 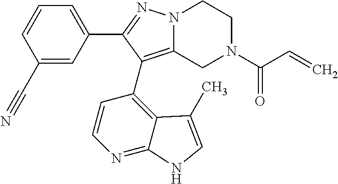 [SMILES: C=CC(=O)N1CCn2nc(-c3cccc(C#N)c3)c(-c3ccnc4[nH]cc(C)c34)c2C1] | ¹H-NMR (400 MHz, DMSO-d6) δ [ppm]: 1.231 (1.20), 1.698 (16.00), 2.144 (0.43), 2.518 (3.44), 2.523 (2.29), 4.098 (2.43), 4.113 (2.04), 4.134 (2.15), 4.147 (1.29), 4.191 (1.48), 4.227 (0.75), 4.307 (1.27), 4.361 (3.97), 4.509 (7.90), 4.714 (0.51), 5.652 (0.45), 5.677 (0.51), 5.767 (1.64), 5.794 (1.65), 6.098 (0.43), 6.151 (1.80), 6.193 (1.72), 6.899 (2.65), 6.911 (2.93), 6.925 (1.71), 6.953 (1.89), 6.968 (1.42), 6.994 (0.95), 7.207 (4.72), 7.382 (2.43), 7.402 (5.42), 7.421 (3.45), 7.551 (4.02), |
| | | 7.571 (3.05), 7.642 (5.04), 7.661 (4.63), |
| | 3-[3-(3-methyl-1H-pyrrolo[2,3- | 7.695 (3.68), 8.227 (0.44), 8.243 (13.58), |
| | b]pyridin-4-yl)-5-(prop-2-enoyl)- | 8.255 (12.82), 11.523 (2.92). |
| | 4,5,6,7-tetrahydropyrazolo[1,5- | LC-MS (Method 1): Rt = 0.80 min; m/z |
| | alpyrazin-2-yl]benzonitrile | 409 (M + H)+ |
| | Produced from 3-[3-(3-methyl-1H- | |
| | pyrrolo[2,3-b]pyridin-4-yl)-4,5,6,7- | |
| | tetrahydropyrazolo[1,5-a]pyrazin-2- | |
| | ylbenzonitrile hydrochloride (1:1) | |
| | (Intermediate 151) | |
| 46 | 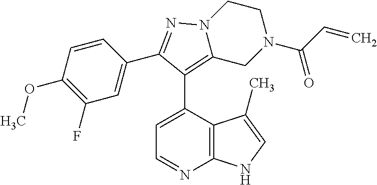 [SMILES: C=CC(=O)N1CCn2nc(-c3ccc(OC)c(F)c3)c(-c3ccnc4[nH]cc(C)c34)c2C1] | ¹H-NMR (400 MHz, DMSO-d6) δ [ppm]: 1.712 (4.96), 2.518 (1.30), 2.523 (0.86), 3.735 (16.00), 4.081 (0.57), 4.096 (0.50), 4.117 (0.61), 4.166 (0.41), 4.311 (1.04), 4.471 (1.31), 5.758 (0.44), 5.786 (0.44), 6.144 (0.47), 6.187 (0.47), 6.868 (0.70), 6.880 (0.77), 6.919 (0.52), 6.944 (0.75), 6.966 (1.20), 6.987 (1.64), 6.994 (1.37), 7.124 (0.68), 7.156 (0.60), 7.188 (1.35), 8.224 (3.59), 8.236 (3.37), 11.474 (0.79). LC-MS (Method 1): Rt = 0.82 min; m/z = 432 (M + H)+ |
| | 1-[2-(3-fluoro-4-methoxyphenyl)-3-(3- | |
| | methyl-1H-pyrrolo[2,3-b]pyridin-4-yl)- | |
| | 6,7-dihydropyrazolo[1,5-a]pyrazin- | |
| | 5(4H)-yl]prop-2-en-1-one | |
| | Produced from 2-(3-fluoro-4- | |
| | methoxyphenyl)-3-(3-methyl-1H- | |
| | pyrrolo[2,3-b]pyridin-4-yl)-4,5,6,7- | |
| | tetrahydropyrazolo[1,5-a]pyrazine | |
| | hydrochloride (1:1) (Intermediate 152) | |
| 47 | 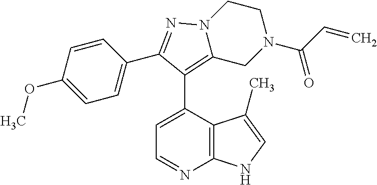 [SMILES: C=CC(=O)N1CCn2nc(-c3ccc(OC)cc3)c(-c3ccnc4[nH]cc(C)c34)c2C1] | ¹H-NMR (400 MHz, DMSO-d6) δ [ppm]: 1.718 (4.47), 2.518 (1.45), 2.523 (0.95), 3.652 (16.00), 3.673 (0.46), 3.678 (0.51), 4.077 (0.52), 4.092 (0.48), 4.113 (0.57), 4.300 (0.98), 4.459 (1.71), 5.755 (0.40), 5.782 (0.41), 6.142 (0.45), 6.184 (0.44), 6.731 (1.85), 6.753 (1.87), 6.843 (0.66), 6.855 (0.69), 7.163 (1.22), 7.246 (1.54), 7.266 (1.14), 8.202 (3.20), 8.214 (2.96), 11.433 (0.73). LC-MS (Method 1): Rt = 0.78 min; m/z = 414 (M + H)+ |
| | 1-[2-(4-methoxyphenyl)-3-(3-methyl- | |
| | 1H-pyrrolo[2,3-b]pyridin-4-yl)-6,7- | |
| | dihydropyrazolo[1,5-a]pyrazin-5(4H)- | |
| | yl]prop-2-en-1-one | |
| | Produced from 2-(4-methoxyphenyl)- | |
| | 3-(3-methyl-1H-pyrrolo[2,3-b]pyridin- | |
| | 4-yl)-4,5,6,7-tetrahydropyrazolo[1,5- | |
| | a]pyrazine hydrochloride (1:1) | |
| | (Intermediate 153) | |
| 48 | 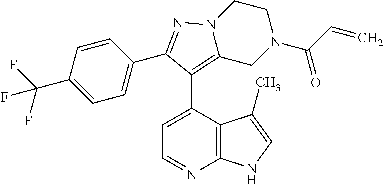 [SMILES: C=CC(=O)N1CCn2nc(-c3ccc(C(F)(F)F)cc3)c(-c3ccnc4[nH]cc(C)c34)c2C1] | ¹H-NMR (400 MHz, DMSO-d6) δ [ppm]: 1.656 (0.51), 1.706 (16.00), 2.074 (0.54), 2.518 (4.31), 2.523 (2.76), 4.101 (2.18), 4.115 (1.85), 4.136 (2.19), 4.149 (1.33), 4.192 (1.48), 4.227 (0.75), 4.311 (1.27), 4.367 (3.79), 4.493 (7.60), 4.694 (0.51), 5.673 (0.48), 5.765 (1.60), 5.792 (1.62), 6.150 (1.81), 6.192 (1.73), 6.865 (0.41), 6.886 (2.56), 6.896 (2.78), 6.926 (1.71), 6.952 (1.48), 6.968 (1.18), 6.994 (0.95), 7.196 (4.51), 7.527 (4.28), 7.548 (7.60), 7.565 (10.16), 7.585 (3.29), 8.136 (0.80), |
| | | 8.233 (12.84), 8.245 (12.23), 11.505 |
| | 1-[3-(3-methyl-1H-pyrrolo[2,3- | (2.84). |
| | b]pyridin-4-yl)-2-[4- | LC-MS (Method 1): Rt = 1.02 min; m/z |
| | (trifluoromethyl)phenyl]-6,7- | 452 (M + H)+ |
| | dihydropyrazolo[1,5-a]pyrazin-5(4H)- | |
| | yl]prop-2-en-1-one | |
| | Produced from 3-(3-methyl-1H- | |
| | pyrrolo[2,3-b]pyridin-4-yl)-2-[4- | |
| | (trifluoromethyl)phenyl]-4,5,6,7- | |
| | tetrahydropyrazolo[1,5-a]pyrazine | |
| | hydrochloride (1:1) (Intermediate 154) | |
| 49 | 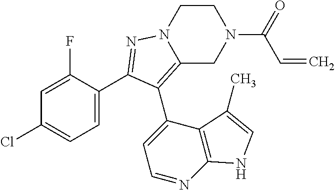 [SMILES: C=CC(=O)N1CCn2nc(-c3ccc(Cl)cc3F)c(-c3ccnc4[nH]cc(C)c34)c2C1] | ¹H-NMR (400 MHz, DMSO-d6) δ [ppm]: 0.931 (0.43), 0.950 (1.01), 0.969 (0.53), 1.232 (1.10), 1.795 (16.00), 2.075 (0.77), 2.518 (11.96), 2.523 (7.90), 4.101 (2.88), 4.155 (0.94), 4.172 (0.60), 4.202 (1.70), 4.238 (1.09), 4.350 (4.39), 4.421 (1.68), 4.464 (2.79), 4.565 (3.48), 4.606 (2.07), 4.677 (0.64), 5.675 (0.62), 5.765 (2.08), 5.792 (2.06), 6.100 (0.53), 6.151 (2.29), 6.193 (2.18), 6.661 (0.53), 6.711 (3.24), 6.723 (3.32), 6.776 (0.91), 6.833 (0.54), 6.855 (0.51), 6.924 (1.41), 6.951 (1.49), |
| | | 6.966 (1.38), 6.993 (1.16), 7.162 (5.75), |
| | 1-[2-(4-chloro-2-fluorophenyl)-3-(3- | 7.188 (3.34), 7.207 (3.79), 7.307 (6.21), |
| | methyl-1H-pyrrolo[2,3-b]pyridin-4-yl)- | 7.311 (6.08), 7.331 (6.92), 7.336 (7.12), |
| | 6,7-dihydropyrazolo[1,5-a]pyrazin- | 7.362 (1.38), 7.800 (0.48), 7.822 (0.46), |
| | 5(4H)-yl]prop-2-en-1-one | 8.117 (10.15), 8.130 (9.49), 11.410 (3.81). |
| | Produced from 2-(4-chloro-2- | LC-MS (Method 1): Rt = 0.88 min; m/z = |
| | fluorophenyl)-3-(3-methyl-1H- | 436 (M + H)+ |
| | pyrrolo[2,3-b]pyridin-4-yl)-4,5,6,7- | |
| | tetrahydropyrazolo[1,5-a]pyrazine | |
| | trifluoroacetic acid (Intermediate 146) | |
| 50 | 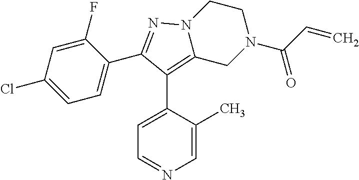 [SMILES: C=CC(=O)N1CCn2nc(-c3ccc(Cl)cc3F)c(-c3ccncc3C)c2C1] | ¹H-NMR (400 MHz, DMSO-d6) δ [ppm]: 1.903 (16.00), 2.337 (0.44), 2.518 (3.81), 2.523 (2.47), 2.678 (0.44), 4.108 (0.72), 4.155 (0.65), 4.194 (0.59), 4.276 (0.63), 4.330 (1.62), 4.607 (0.68), 4.669 (0.77), 5.772 (0.70), 5.799 (0.68), 6.162 (0.87), 6.202 (0.78), 6.924 (0.52), 6.949 (0.49), 6.965 (0.45), 7.089 (1.06), 7.100 (1.25), 7.287 (1.42), 7.291 (1.45), 7.307 (2.08), 7.312 (2.15), 7.379 (2.94), 7.384 (2.46), 7.404 (2.45), 7.409 (2.07), 8.363 (1.80), |
| | | 8.375 (1.50), 8.436 (2.45). |
| | 1-[2-(4-chloro-2-fluorophenyl)-3-(3- | LC-MS (Method 1): Rt = 0.73 min; m/z = |
| | methylpyridin-4-yl)-6,7- | 397 (M + H)+ |
| | dihydropyrazolo[1,5-a]pyrazin-5(4H)- | |
| | yl]prop-2-en-1-one | |
| | Produced from 2-(4-chloro-2- | |
| | fluorophenyl)-3-(3-methylpyridin-4-yl)- | |
| | 4,5,6,7-tetrahydropyrazolo[1,5- | |
| | a]pyrazine trifluoroacetate (1:1) | |
| | (Intermediate 147) | |
| 51 | 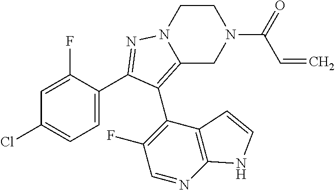 [SMILES: C=CC(=O)N1CCn2nc(-c3ccc(Cl)cc3F)c(-c3c(F)cnc4[nH]ccc34)c2C1] | ¹H-NMR (400 MHz, DMSO-d6) δ [ppm]: 1.232 (0.68), 2.075 (8.89), 2.518 (16.00), 2.523 (10.33), 2.660 (1.21), 4.217 (3.40), 4.374 (3.79), 4.611 (1.22), 4.653 (2.45), 4.728 (2.67), 4.772 (1.87), 4.814 (0.86), 5.682 (0.59), 5.768 (1.79), 5.795 (1.73), 5.975 (3.53), 6.150 (2.24), 6.192 (1.93), 6.925 (1.18), 6.953 (1.22), 6.967 (1.15), 6.994 (0.97), 7.267 (4.44), 7.287 (10.09), 7.309 (3.56), 7.446 (5.12), 7.463 (6.06), 7.483 (2.16), 7.645 (0.48), 8.207 (10.61), 8.213 (9.96), 8.313 (0.49), 8.319 (0.61), |
| | | 11.830 (2.74). |
| | 1-[2-(4-chloro-2-fluorophenyl)-3-(5- | LC-MS (Method 1): Rt = 0.99 min; m/z = |
| | fluoro-1H-pyrrolo[2,3-b]pyridin-4-yl)- | 440 (M + H)+ |
| | 6,7-dihydropyrazolo[1,5-a]pyrazin- | |
| | 5(4H)-yl]prop-2-en-1-one | |
| | Produced from 2-(4-chloro-2- | |
| | fluorophenyl)-3-(5-fluoro-1H- | |
| | pyrrolo[2,3-b]pyridin-4-yl)-4,5,6,7- | |
| | tetrahydropyrazolo[1,5-a]pyrazine | |
| | trifluoroacetate (1:1) (Intermediate | |
| | 148) | |
| 52 | 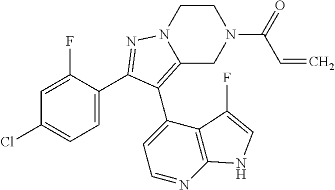 [SMILES: C=CC(=O)N1CCn2nc(-c3ccc(Cl)cc3F)c(-c3ccnc4[nH]cc(F)c34)c2C1] | ¹H-NMR (400 MHz, DMSO-d6) δ [ppm]: 1.232 (0.41), 2.075 (13.20), 2.518 (16.00), 2.523 (10.16), 4.026 (0.79), 4.135 (1.74), 4.222 (2.96), 4.258 (1.97), 4.305 (2.54), 4.350 (4.89), 4.703 (0.53), 4.753 (9.32), 4.827 (0.97), 4.934 (0.96), 4.972 (0.53), 5.694 (0.90), 5.767 (2.52), 5.792 (2.46), 6.148 (2.93), 6.189 (2.98), 6.841 (3.74), 6.852 (4.08), 6.891 (1.50), 6.922 (1.72), 6.948 (1.78), 6.963 (1.63), 6.991 (1.33), 7.219 (4.89), 7.240 (6.17), 7.283 (6.40), 7.288 (5.77), 7.309 (6.85), 7.313 (6.65), |
| | | 7.331 (8.84), 7.359 (3.43), 7.378 (4.19), |
| | 1-[2-(4-chloro-2-fluorophenyl)-3-(3- | 7.399 (1.78), 8.224 (10.16), 8.236 (9.62), |
| | fluoro-1H-pyrolo[2,3-b]pyridin-4-yl)- | 11.534 (4.38). |
| | 6,7-dihydropyrazolo[1,5-a]pyrazin- | LC-MS (Method 1): Rt = 0.95 min; m/z = |
| | 5(4H)-yl]prop-2-en-1-one | 440 (M + H)+ |
| | Produced from 2-(4-chloro-2- | |
| | fluorophenyl)-3-(3-fluoro-1H- | |
| | pyrrolo[2,3-b]pyridin-4-yl)-4,5,6,7- | |
| | tetrahydropyrazolo[1,5-a]pyrazine | |
| | trifluoroacetate (1:1) (Intermediate | |
| | 149) | |
| 53 | 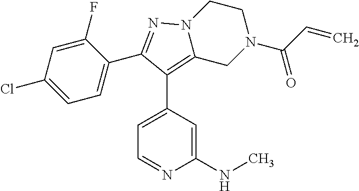 [SMILES: C=CC(=O)N1CCn2nc(-c3ccc(Cl)cc3F)c(-c3ccnc(NC)c3)c2C1] | ¹H-NMR (400 MHz, DMSO-d6) δ [ppm]: 1.232 (0.82), 2.318 (0.60), 2.323 (1.35), 2.327 (1.88), 2.331 (1.31), 2.337 (0.57), 2.518 (5.48), 2.523 (3.56), 2.665 (2.53), 2.673 (16.00), 2.685 (14.64), 4.095 (0.83), 4.169 (2.31), 4.282 (2.38), 4.869 (3.73), 4.969 (0.96), 5.774 (1.24), 5.800 (1.12), 6.117 (7.13), 6.159 (2.27), 6.209 (1.57), 6.459 (1.18), 6.926 (0.74), 6.952 (0.76), 6.967 (0.67), 6.994 (0.55), 7.344 (3.15), 7.350 (3.23), 7.365 (4.29), 7.370 (4.59), 7.445 (7.16), 7.451 (4.21), 7.466 (5.08), |
| | | 7.470 (4.70), 7.476 (3.98), 7.486 (2.14), |
| | 1-[2-(4-chloro-2-fluorophenyl)-3-[2- | 7.881 (4.77), 7.894 (4.60). |
| | (methylamino)pyridin-4-yl]-6,7- | LC-MS (Method 1): Rt = 0.67 min; m/z = |
| | dihydropyrazolo[1,5-a]pyrazin-5(4H)- | 412 (M + H)+ |
| | yl]prop-2-en-1-one | |
| | Produced from 4-[2-(4-chloro-2- | |
| | fluorophenyl)-4,5,6,7- | |
| | tetrahydropyrazolo[1,5-a]pyrazin-3-yl]- | |
| | N-methylpyridin-2-amine | |
| | trifluoroacetate (1:1) (Intermediate | |
| | 150) | |
| 54 | 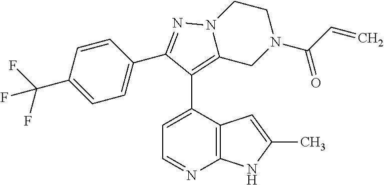 [SMILES: C=CC(=O)N1CCn2nc(-c3ccc(C(F)(F)F)cc3)c(-c3ccnc4[nH]c(C)cc34)c2C1] | 1H-NMR (400 MHz, DMSO-d6) delta [ppm]: 0.905 (0.42), 1.232 (0.92), 2.084 (1.68), 2.133 (0.46), 2.309 (16.00), 2.323 (3.42), 2.327 (4.10), 2.332 (2.77), 2.337 (1.23), 2.518 (10.54), 2.523 (7.28), 2.660 (1.17), 2.665 (2.63), 2.669 (3.70), 2.674 (2.52), 2.679 (1.12), 3.290 (0.53), 3.298 (0.73), 4.123 (0.85), 4.188 (2.45), 4.298 (0.98), 4.353 (2.69), 4.632 (4.11), 4.737 (1.00), 5.775 (3.52), 6.138 (1.31), 6.179 (1.28), 6.767 (0.47), 6.783 (1.85), 6.795 (1.90), 6.833 (0.53), 6.917 (0.77), 6.943 |
| | | (0.82), 6.958 (0.77), 6.984 (0.64), 7.328 |
| | 1-[3-(2-methyl-1H-pyrrolo[2,3- | (0.88), 7.341 (0.78), 7.516 (3.84), 7.537 |
| | b]pyridin-4-yl)-2-[4- | (4.32), 7.599 (7.37), 7.620 (4.39), 8.075 |
| | (trifluoromethyl)phenyl]-6,7- | (0.63), 8.088 (1.93), 8.093 (9.11), 8.105 |
| | dihydropyrazolo[1,5-a]pyrazin-5(4H)- | (8.39), 11.612 (2.24). |
| | yl]prop-2-en-1-one | LC-MS (Method 1): Rt = 0.98 min; m/z = |
| | Produced from 3-(2-methyl-1H- | 452 (M + H)+ |
| | pyrrolo[2,3-b]pyridin-4-yl)-2-[4- | |
| | (trifluoromethyl)phenyl]-4,5,6,7- | |
| | tetrahydropyrazolo[1,5-a]pyrazine | |
| | hydrochloride (1:1) (Intermediate 155) | |
| 55 | 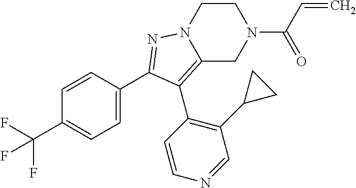 [SMILES: C=CC(=O)N1CCn2nc(-c3ccc(C(F)(F)F)cc3)c(-c3ccncc3C3CC3)c2C1] | ¹H-NMR (400 MHz, DMSO-d6) δ [ppm]: 0.508 (2.47), 0.523 (2.39), 0.544 (2.05), 0.754 (2.20), 0.765 (3.58), 0.780 (3.19), 0.803 (1.82), 0.836 (1.47), 1.527 (2.55), 2.518 (7.32), 2.523 (4.54), 4.152 (2.05), 4.191 (1.79), 4.228 (0.78), 4.288 (1.66), 4.334 (3.59), 4.344 (4.38), 4.580 (0.58), 4.634 (7.05), 4.704 (0.90), 4.771 (0.75), 5.730 (0.71), 5.775 (1.87), 5.801 (1.80), 6.125 (0.62), 6.166 (2.31), 6.206 (1.94), 6.796 (0.43), 6.929 (1.15), 6.955 (1.25), 6.971 (1.17), 6.996 (0.97), 7.175 (2.58), |
| | | 7.186 (2.86), 7.222 (0.99), 7.505 (6.38), |
| | 1-[3-(3-cyclopropylpyridin-4-yl)-2-[4- | 7.525 (5.92), 7.654 (10.04), 7.675 (7.65), |
| | (trifluoromethyl)phenyl]-6,7- | 8.209 (16.00), 8.407 (14.77), 8.419 |
| | dihydropyrazolo[1,5-a]pyrazin-5(4H)- | (13.37). |
| | yl]prop-2-en-1-one | LC-MS (Method 1): Rt = 0.91 min; m/z = |
| | Produced from 3-(3- | 439 (M + H)+ |
| | cyclopropylpyridin-4-yl)-2-[4- | |
| | (trifluoromethyl)phenyl]-4,5,6,7- | |
| | tetrahydropyrazolo[1,5-a]pyrazine | |
| | hydrochloride (1:1) (Intermediate 156) | |
| 56 | 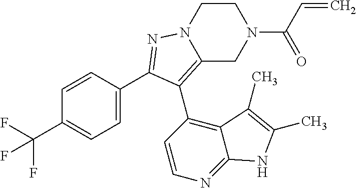 [SMILES: C=CC(=O)N1CCn2nc(-c3ccc(C(F)(F)F)cc3)c(-c3ccnc4[nH]c(C)c(C)c34)c2C1] | ¹H-NMR (400 MHz, DMSO-d6) δ [ppm]: 1.632 (11.67), 2.075 (1.71), 2.249 (16.00), 2.518 (7.05), 2.523 (4.78), 3.284 (0.42), 3.301 (0.73), 3.378 (0.93), 3.382 (0.63), 4.131 (1.62), 4.178 (1.29), 4.362 (2.98), 4.462 (6.11), 4.818 (0.51), 5.769 (1.62), 5.775 (1.16), 5.795 (1.51), 5.801 (0.96), 6.147 (1.47), 6.179 (1.06), 6.185 (1.55), 6.221 (0.68), 6.226 (0.64), 6.797 (2.04), 6.809 (2.20), 6.848 (0.61), 6.922 (0.97), 6.949 (1.04), 6.964 (0.97), 6.990 (0.83), 7.540 (3.19), 7.560 (8.08), 7.570 (10.14), |
| | | 7.591 (2.49), 7.742 (0.97), 7.763 (1.16), |
| | 1-[3-(2,3-dimethyl-1H-pyrrolo[2,3- | 7.981 (0.62), 7.999 (0.55), 8.112 (9.10), |
| | b]pyridin-4-yl)-2-[4- | 8.124 (8.37), 11.438 (2.93). |
| | (trifluoromethyl)phenyl]-6,7- | LC-MS (Method 1): Rt = 1.01 min; m/z = |
| | dihydropyrazolo[1,5-a]pyrazin-5(4H)- | 466 (M + H)+ |
| | yl]prop-2-en-1-one | |
| | Produced from 3-(2,3-dimethyl-1H- | |
| | pyrrolo[2,3-b]pyridin-4-yl)-2-[4- | |
| | (trifluoromethyl)phenyl]-4,5,6,7- | |
| | tetrahydropyrazolo[1,5-a]pyrazine | |
| | hydrochloride (1:1) (Intermediate 163) | |

The following examples were prepared analogous to the preparation of Example 25 starting from the corresponding intermediates by reacting with (2E)-4-(dimethylamino)but-2-enoic acid.

| | Structure | |
|---|---|---|
| Example | Name | Analytical Data |
| 57 | 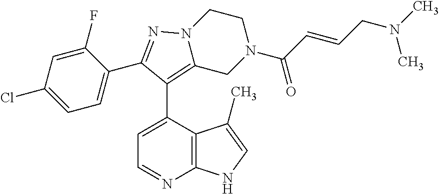 [SMILES: Cc1c[nH]c2nccc(-c3c(-c4ccc(Cl)cc4F)nn4c3CN(C(=O)/C=C/CN(C)C)CC4)c12] | ¹H-NMR (400 MHz, DMSO-d6) δ [ppm]: 1.232 (1.34), 1.790 (11.91), 2.056 (4.10), 2.158 (16.00), 2.518 (7.12), 2.523 (4.57), 2.953 (1.06), 3.053 (3.08), 3.590 (1.81), 3.681 (0.45), 3.701 (0.44), 4.100 (1.06), 4.179 (1.54), 4.289 (1.25), 4.344 (2.89), 4.405 (1.07), 4.448 (1.65), 4.545 (3.40), 4.586 (2.70), 4.629 (0.52), 4.643 (1.01), 6.437 (0.45), 6.545 (0.43), 6.635 (1.00), 6.674 (1.34), 6.721 (2.63), 6.745 (2.57), 6.782 (0.93), 7.158 (4.45), 7.184 (2.90), 7.208 (3.70), 7.305 (6.01), 7.310 (6.02), |
| | | 7.330 (6.89), 7.335 (7.50), 7.356 (1.27), |
| | (2E)-1-[2-(4-chloro-2-fluorophenyl)-3- | 8.113 (12.57), 8.125 (11.79), 11.402 |
| | (3-methyl-1H-pyrrolo[2,3-b]pyridin-4- | (3.99). |
| | yl)-6,7-dihydropyrazolo[1,5-a]pyrazin- | LC-MS (Method 1): Rt = 0.68 min; m/z = |
| | 5(4H)-yl]-4-(dimethylamino)but-2-en- | 493 (M + H)+ |
| | 1-one | |
| | Produced from 2-(4-chloro-2- | |
| | fluorophenyl)-3-(3-methyl-1H- | |
| | pyrrolo[2,3-b]pyridin-4-yl)-4,5,6,7- | |
| | tetrahydropyrazolo[1,5-a]pyrazine | |
| | hydrochloride (1:1) (Intermediate 146) | |
| 58 | 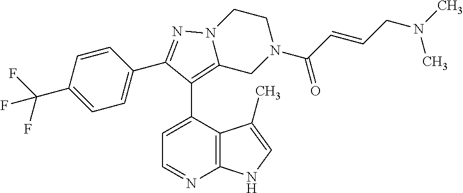 [SMILES: Cc1c[nH]c2nccc(-c3c(-c4ccc(C(F)(F)F)cc4)nn4c3CN(C(=O)/C=C/CN(C)C)CC4)c12] | ¹H-NMR (400 MHz, DMSO-d6) δ [ppm]: 1.700 (15.97), 2.052 (4.18), 2.158 (16.00), 2.518 (7.78), 2.523 (5.00), 2.951 (1.06), 3.053 (3.08), 4.115 (1.52), 4.168 (1.42), 4.304 (1.26), 4.362 (3.14), 4.477 (5.96), 4.648 (0.49), 6.438 (0.47), 6.635 (0.88), 6.673 (1.26), 6.746 (1.72), 6.784 (0.86), 6.894 (2.73), 7.192 (4.60), 7.523 (3.58), 7.543 (7.05), 7.563 (11.73), 7.585 (4.13), 8.146 (0.42), 8.229 (10.00), 8.241 (9.24), 11.499 (3.39). LC-MS (Method 1): Rt = 0.78 min; m/z = |
| | | 509 (M + H)+ |
| | (2E)-4-(dimethylamino)-1-[3-(3- | |
| | methyl-1H-pyrrolo[2,3-b]pyridin-4-yl)- | |
| | 2-[4-(trifluoromethyl)phenyl]-6,7- | |
| | dihydropyrazolo[1,5-a]pyrazin-5(4H)- | |
| | yl]but-2-en-1-one | |
| | Produced from 3-(3-methyl-1H- | |
| | pyrrolo[2,3-b]pyridin-4-yl)-2-[4- | |
| | (trifluoromethyl)phenyl]-4,5,6,7- | |
| | tetrahydropyrazolo[1,5-a]pyrazine | |
| | hydrochloride (1:1) (Intermediate 154) | |
| 59 | 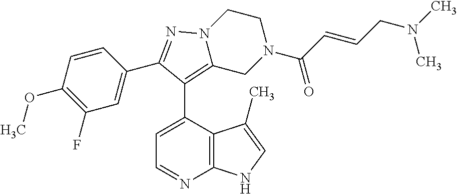 | ¹H-NMR (400 MHz, DMSO-d6) δ [ppm]: 1.707 (4.21), 2.040 (1.12), 2.147 (4.29), 2.518 (1.23), 2.523 (0.81), 3.038 (0.81), 3.051 (0.75), 3.734 (16.00), 4.095 (0.40), 4.306 (0.76), 4.454 (1.07), 6.735 (0.43), 6.867 (0.58), 6.878 (0.72), 6.943 (0.46), 6.965 (1.19), 6.985 (1.40), 6.992 (1.45), 7.120 (0.50), 7.153 (0.47), 7.184 (1.22), 8.220 (2.41), 8.232 (2.27), 11.468 (0.85). LC-MS (Method 1): Rt = 0.63 min; m/z = 489 (M + H)+ |
| | (2E)-4-(dimethylamino)-1-[2-(3-fluoro- | |
| | 4-methoxyphenyl)-3-(3-methyl-1H- | |
| | pyrrolo[2,3-b]pyridin-4-yl)-6,7- | |
| | dihydropyrazolo[1,5-a]pyrazin-5(4H)- | |
| | yl]but-2-en-1-one | |
| | Produced from 2-(3-fluoro-4- | |
| | methoxyphenyl)-3-(3-methyl-1H- | |
| | pyrrolo[2,3-b]pyridin-4-yl)-4,5,6,7- | |
| | tetrahydropyrazolo[1,5-a]pyrazine | |
| | hydrochloride (1:1) (Intermediate 152) | |
| 60 | 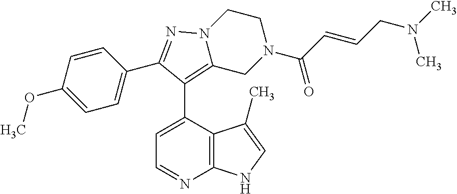 | 1H-NMR (400 MHz, DMSO-d6) delta [ppm]: 1.108 (0.19), 1.713 (3.88), 2.038 (1.09), 2.147 (4.18), 2.518 (0.89), 2.523 (0.59), 2.934 (0.27), 3.038 (0.79), 3.051 (0.73), 3.652 (16.00), 4.092 (0.39), 4.139 (0.36), 4.239 (0.30), 4.295 (0.75), 4.442 (1.38), 6.626 (0.20), 6.666 (0.31), 6.730 (2.41), 6.751 (2.30), 6.842 (0.57), 6.853 (0.69), 7.159 (1.16), 7.243 (1.38), 7.263 (1.13), 8.198 (2.23), 8.210 (2.09), 11.427 (0.86). LC-MS (Method 1): Rt = 0.59 min; m/z = |
| | | 471 (M + H)+ |
| | (2E)-4-(dimethylamino)-1-[2-(4- | |
| | methoxyphenyl)-3-(3-methyl-1H- | |
| | pyrrolo[2,3-b]pyridin-4-yl)-6,7- | |
| | dihydropyrazolo[1,5-a]pyrazin-5(4H)- | |
| | yl]but-2-en-1-one | |
| | Produced from 2-(4-methoxyphenyl)- | |
| | 3-(3-methyl-1H-pyrrolo[2,3-b]pyridin- | |
| | 4-yl)-4,5,6,7-tetrahydropyrazolo[1,5- | |
| | a]pyrazine hydrochloride (1:1) | |
| | (Intermediate 153) | |
| 61 | 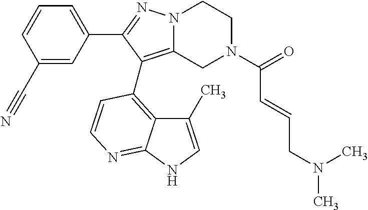 [SMILES: Cc1c[nH]c2nccc(-c3c(-c4cccc(C#N)c4)nn4c3CN(C(=O)/C=C/CN(C)C)CC4)c12] | ¹H-NMR (400 MHz, DMSO-d6) δ [ppm]: 1.232 (0.46), 1.693 (14.32), 2.047 (4.28), 2.152 (16.00), 2.523 (8.72), 2.947 (1.09), 3.046 (3.14), 4.116 (1.40), 4.170 (1.47), 4.355 (3.02), 4.492 (5.13), 4.667 (0.49), 6.446 (0.49), 6.548 (0.64), 6.635 (0.91), 6.674 (1.24), 6.745 (1.64), 6.910 (2.59), 7.204 (4.41), 7.382 (2.64), 7.402 (5.88), 7.421 (3.73), 7.549 (4.56), 7.569 (3.57), 7.642 (4.87), 7.661 (5.04), 7.693 (2.91), 8.239 (8.47), 8.251 (7.95), 11.516 (3.08). LC-MS (Method 1): Rt = 0.59 min; m/z = 466 (M + H)+ |
| | 3-{5-[(2E)-4-(dimethylamino)but-2- | |
| | enoyl]-3-(3-methyl-1H-pyrrolo[2,3- | |
| | b]pyridin-4-yl)-4,5,6,7- | |
| | tetrahydropyrazolo[1,5-a]pyrazin-2- | |
| | yl}benzonitrile | |
| | Produced from 3-[3-(3-methyl-1H- | |
| | pyrrolo[2,3-b]pyridin-4-yl)-4,5,6,7- | |
| | tetrahydropyrazolo[1,5-a]pyrazin-2- | |
| | yl]benzonitrile hydrochloride (1:1) | |
| | (Intermediate 151) | |

### Example 62

### 2-[(dimethylamino)methyl]-1-[2-(4-fluorophenyl)-3-(pyridin-4-yl)-6,7-dihydropyrazolo[1,5-a]pyrazin-5(4H)-yl]prop-2-en-1-one

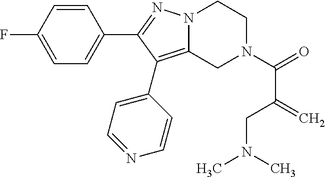 [SMILES: C=C(CN(C)C)C(=O)N1CCn2nc(-c3ccc(F)cc3)c(-c3ccncc3)c2C1]

2-(4-fluorophenyl)-3-(pyridin-4-yl)-4,5,6,7-tetrahydropyrazolo[1,5-a]pyrazine (50.0 mg, 170 μmol, Intermediate 3) was dissolved in DMF (2.1 ml), then 2-[(dimethylamino)methyl]prop-2-enoic acid (26.3 mg, 204 μmol; CAS-RN: [5415-98-5]), then N,N-diisopropylethylamine (180 μl, 1.0 mmol; CAS-RN: [7087-68-5]) and then 2,4,6-Tripropyl-1,3,5,2,4,6-trioxatriphosphorinane-2,4,6-trioxide solution ((162 mg, 50% purity in DMF, 255 μmol), 170 μmol) were added, and the mixture was stirred at rt for 1 h. Water (0.1 ml) was added the mixture was purified by reverse phase preparative HPLC (acidic conditions) yielding the title compound 18.9 mg (90% purity, 25% yield).

LC-MS (Method 1): Rₜ=0.60 min; MS (ESIpos): m/z=406.2 [M+H]⁺

¹H-NMR (500 MHz, DMSO-d6) δ [ppm]: 2.128 (1.90), 2.514 (0.42), 3.058 (1.03), 3.074 (16.00), 4.087 (0.70), 4.099 (0.51), 4.250 (0.44), 4.262 (0.62), 4.815 (1.75), 5.312 (0.44), 5.418 (0.42), 7.139 (1.28), 7.142 (0.77), 7.148 (0.56), 7.151 (0.99), 7.156 (1.10), 7.175 (0.59), 7.390 (0.64), 7.401 (0.62), 7.408 (0.56), 7.418 (0.50), 8.532 (0.96), 8.535 (0.56), 8.541 (0.58), 8.544 (0.93).

### Example 63

### (2E)-1-[2-(4-fluorophenyl)-3-(pyridin-4-yl)-6,7-dihydropyrazolo[1,5-a]pyrazin-5(4H)-yl]-4-(morpholin-4-yl) but-2-en-1-one

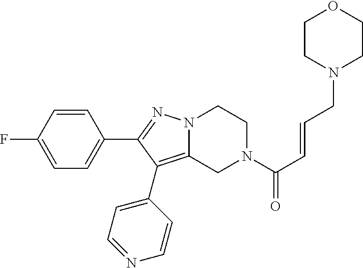 [SMILES: O=C(/C=C/CN1CCOCC1)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine (56.0 mg, 190 μmol, Intermediate 3) and (2E)-4-(morpholin-4-yl) but-2-enoic acid-hydrogen chloride (1/1) (59.3 mg, 285 μmol; CAS-RN: [1807940-64-2]) as the starting materials, 40.4 mg (98% purity, 46% yield) of the desired product were prepared after purification by preparative HPLC (basic conditions).

LC-MS (Method 1): Rₜ=0.4 min; MS (ESIpos): m/z=448.2 [M+H]⁺

¹H-NMR (400 MHz, DMSO-d6) δ [ppm]: 2.318 (3.11), 2.323 (3.81), 2.327 (4.38), 2.332 (3.88), 2.337 (3.28), 2.365 (5.50), 2.518 (8.38), 2.523 (5.86), 2.660 (0.58), 2.665 (1.38), 2.669 (1.89), 2.673 (1.33), 2.678 (0.59), 3.112 (3.43), 3.521 (2.71), 3.575 (6.60), 4.080 (1.32), 4.136 (3.05), 4.278 (3.19), 4.821 (4.28), 4.943 (1.37), 6.642 (2.31), 6.675 (1.60), 6.767 (1.50), 6.805 (0.83), 7.164 (6.93), 7.174 (10.01), 7.180 (4.30), 7.192 (4.02), 7.197 (16.00), 7.202 (3.72), 7.214 (3.03), 7.219 (9.28), 7.226 (1.17), 7.352 (4.45), 7.366 (5.65), 7.386 (3.36), 8.539 (11.49), 8.554 (10.91).

### Example 64

### 1-[2-(4-fluorophenyl)-3-(pyridin-4-yl)-6,7-dihydropyrazolo[1,5-a]pyrazin-5(4H)-yl]-2-[(morpholin-4-yl)methyl]prop-2-en-1-one

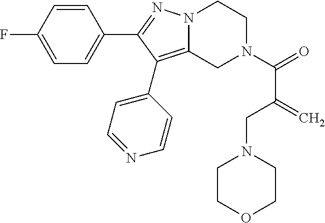 [SMILES: C=C(CN1CCOCC1)C(=O)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine (56.0 mg, 190 μmol, Intermediate 3) and 2-[(morpholin-4-yl)methyl]prop-2-enoic acid (34.9 mg, 204 μmol; CAS-RN: [4432-44-4]) as the starting materials, 11.4 mg (86% purity, 13% yield) of the desired product were prepared after purification by preparative HPLC (basic conditions).

LC-MS (Method 1): Rₜ=0.42 min; MS (ESIpos): m/z=448.2 [M+H]⁺

¹H-NMR (400 MHz, DMSO-d6) δ [ppm]: 2.085 (0.51), 2.146 (1.39), 2.323 (2.23), 2.327 (2.91), 2.332 (2.30), 2.336 (1.49), 2.393 (2.74), 2.518 (10.71), 2.523 (7.26), 2.563 (0.59), 2.660 (0.78), 2.665 (1.66), 2.669 (2.23), 2.673 (1.59), 2.679 (0.75), 3.110 (2.00), 3.257 (1.06), 3.375 (1.30), 3.461 (1.35), 3.547 (2.90), 3.743 (0.92), 4.069 (3.99), 4.169 (0.65), 4.181 (0.56), 4.266 (3.08), 4.834 (5.13), 5.240 (0.67), 5.400 (1.23), 5.464 (1.14), 5.479 (1.06), 7.090 (1.54), 7.094 (1.33), 7.101 (1.40), 7.105 (1.71), 7.159 (2.57), 7.164 (2.47), 7.176 (2.16), 7.183 (7.77), 7.188 (3.60), 7.200 (4.13), 7.206 (16.00), 7.211 (3.59), 7.222 (3.79), 7.228 (9.23), 7.235 (1.26), 7.241 (0.75), 7.361 (4.25), 7.374 (5.10), 7.395 (3.36), 7.790 (0.48), 8.497 (0.79), 8.513 (0.97), 8.535 (6.20), 8.548 (5.98), 8.576 (0.49).

### Example 65

### 4-(dimethylamino)-1-[2-(4-fluorophenyl)-3-(pyridin-4-yl)-6,7-dihydropyrazolo[1,5-a]pyrazin-5(4H)-yl]but-2-yn-1-one

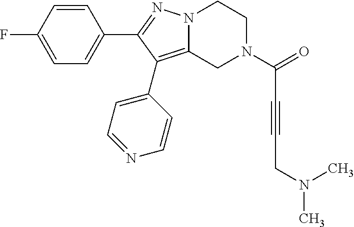 [SMILES: CN(C)CC#CC(=O)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine (56.0 mg, 190 μmol, Intermediate 3) and 4-(dimethylamino)but-2-ynoic acid (25.9 mg, 204 μmol) as the starting materials, 22.0 mg (98% purity, 31% yield) of the desired product were prepared after purification by preparative HPLC (basic conditions).

LC-MS (Method 1): Rₜ=0.40 min; MS (ESIpos): m/z=404.1 [M+H]⁺

¹H-NMR (400 MHz, DMSO-d6) δ [ppm]: 1.942 (14.36), 2.239 (16.00), 2.518 (1.73), 2.523 (1.18), 3.435 (4.20), 3.526 (4.25), 4.080 (0.54), 4.093 (1.11), 4.108 (0.78), 4.226 (0.79), 4.241 (1.12), 4.255 (0.63), 4.274 (1.00), 4.287 (0.76), 4.340 (0.73), 4.353 (0.99), 4.366 (0.41), 4.816 (2.94), 5.119 (2.90), 7.153 (2.64), 7.156 (3.27), 7.160 (1.72), 7.164 (1.80), 7.168 (3.47), 7.171 (2.85), 7.179 (1.34), 7.181 (1.48), 7.186 (0.52), 7.202 (2.88), 7.203 (2.95), 7.219 (0.58), 7.224 (1.74), 7.226 (1.52), 7.359 (1.29), 7.365 (0.55), 7.373 (1.60), 7.376 (1.85), 7.381 (1.69), 7.389 (1.78), 7.395 (1.67), 7.398 (1.27), 7.407 (0.44), 7.412 (1.00), 8.526 (2.46), 8.530 (1.35), 8.540 (3.53), 8.544 (1.79), 8.551 (1.42), 8.555 (2.27).

### Example 66

### (2E)-1-[2-(4-fluorophenyl)-3-(pyridin-4-yl)-6,7-dihydropyrazolo[1,5-a]pyrazin-5(4H)-yl]-4-methoxybut-2-en-1-one

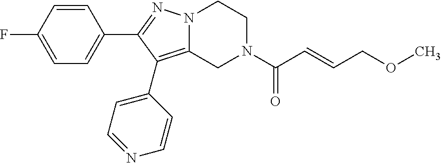 [SMILES: COC/C=C/C(=O)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (60.0 mg, 181 μmol, Intermediate 164) and (2E)-4-methoxybut-2-enoic acid (31.6 mg, 272 μmol; CAS-RN: [75933-65-2]) as the starting materials, 43.0 mg (98% purity, 59% yield) of the desired product were prepared after purification by preparative HPLC (basic conditions).

LC-MS (Method 1): Rₜ=0.60 min; MS (ESIpos): m/z=393 [M+H]⁺

¹H-NMR (400 MHz, DMSO-d6) delta [ppm]: 2.318 (0.60), 2.518 (6.63), 2.523 (5.04), 2.660 (0.60), 2.679 (0.58), 3.238 (1.97), 3.309 (8.97), 4.084 (6.92), 4.128 (3.52), 4.275 (3.47), 4.828 (4.71), 4.943 (1.14), 6.644 (0.46), 6.699 (0.84), 6.708 (1.02), 6.744 (6.89), 7.167 (7.13), 7.175 (10.05), 7.180 (4.37), 7.192 (4.00), 7.197 (16.00), 7.203 (3.50), 7.214 (2.99), 7.219 (8.68), 7.227 (1.07), 7.355 (4.07), 7.368 (5.02), 8.540 (15.14), 8.544 (9.28), 8.551 (9.27), 8.555 (14.38).

### Example 67

### (2E)-4,4,4-trifluoro-1-[2-(4-fluorophenyl)-3-(pyridin-4-yl)-6,7-dihydropyrazolo[1,5-a]pyrazin-5(4H)-yl]but-2-en-1-one

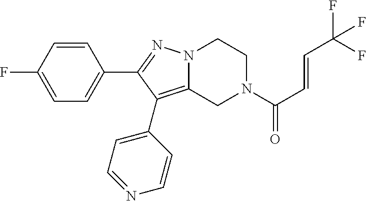 [SMILES: O=C(/C=C/C(F)(F)F)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (60.0 mg, 181 μmol, Intermediate 164) and (2E)-4,4,4-trifluorobut-2-enoic acid (38.1 mg, 272 μmol; CAS-RN: [71027-02-6]) as the starting materials, 48.5 mg (98% purity, 63% yield) of the desired product were prepared after purification by preparative HPLC (basic conditions).

LC-MS (Method 1): Rₜ=0.74 min; MS (ESIpos): m/z=417 [M+H]⁺

¹H-NMR (400 MHz, DMSO-d6) delta [ppm]: 2.518 (6.03), 2.523 (4.41), 3.321 (0.69), 4.108 (2.24), 4.121 (1.78), 4.153 (3.24), 4.166 (6.29), 4.180 (4.26), 4.239 (1.62), 4.252 (2.30), 4.310 (4.49), 4.324 (6.58), 4.336 (3.18), 4.862 (16.00), 5.023 (4.95), 6.802 (1.00), 6.808 (0.77), 6.820 (2.61), 6.838 (2.74), 6.859 (3.20), 6.876 (2.75), 6.893 (0.74), 7.157 (11.29), 7.161 (7.81), 7.168 (8.58), 7.172 (14.80), 7.180 (9.17), 7.185 (5.08), 7.190 (4.60), 7.197 (6.65), 7.202 (13.28), 7.219 (4.36), 7.225 (7.24), 7.332 (1.87), 7.346 (2.47), 7.353 (3.16), 7.361 (7.14), 7.366 (4.26), 7.374 (7.38), 7.383 (5.76), 7.391 (2.49), 7.397 (4.68), 7.444 (1.01), 7.483 (1.00), 7.500 (2.94), 7.505 (2.97), 7.539 (2.64), 7.544 (2.66), 8.533 (3.37), 8.548 (15.08), 8.552 (8.57), 8.560 (7.78), 8.563 (11.07).

### Example 68

### 1-[2-(4-fluorophenyl)-3-(pyridin-4-yl)-6,7-dihydropyrazolo[1,5-a]pyrazin-5(4H)-yl]-4-(morpholin-4-yl) but-2-yn-1-one

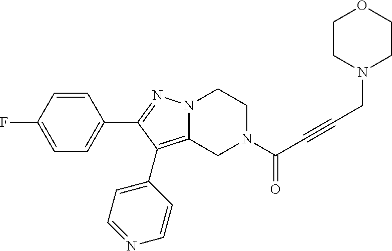 [SMILES: O=C(C#CCN1CCOCC1)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (60.0 mg, 181 μmol, Intermediate 3) and 4-(morpholin-4-yl) but-2-ynoic acid (46.0 mg, 272 μmol; CAS-RN: [38346-95-1]) as the starting materials, 27.0 mg (98% purity, 33% yield) of the desired product were prepared after purification by preparative HPLC (basic conditions).

LC-MS (Method 1): Rₜ=0.51 min; MS (ESIpos): m/z=446 [M+H]⁺

¹H-NMR (400 MHz, DMSO-d6) δ [ppm]: 2.256 (3.22), 2.267 (4.52), 2.278 (3.49), 2.318 (0.56), 2.518 (6.69), 2.523 (4.70), 3.417 (3.80), 3.429 (4.96), 3.440 (3.77), 3.502 (12.93), 3.591 (16.00), 3.598 (5.05), 3.610 (5.91), 3.622 (4.42), 4.072 (1.34), 4.085 (2.72), 4.100 (1.92), 4.226 (1.93), 4.241 (2.91), 4.253 (2.33), 4.263 (3.11), 4.277 (2.54), 4.337 (2.49), 4.351 (2.98), 4.364 (1.28), 4.815 (8.69), 5.087 (7.25), 7.152 (8.69), 7.155 (8.81), 7.159 (4.71), 7.164 (5.24), 7.167 (10.12), 7.170 (7.36), 7.181 (5.02), 7.185 (1.86), 7.197 (2.30), 7.202 (10.99), 7.208 (2.18), 7.219 (1.95), 7.225 (6.37), 7.232 (0.69), 7.352 (0.96), 7.359 (7.28), 7.365 (2.82), 7.373 (7.83), 7.381 (6.91), 7.390 (2.32), 7.395 (5.92), 7.403 (0.56), 8.535 (6.84), 8.540 (11.90), 8.544 (5.28), 8.547 (4.48), 8.551 (10.53), 8.555 (7.89).

### Example 69

### (2E)-4-(diethylamino)-1-[2-(4-fluorophenyl)-3-(pyridin-4-yl)-6,7-dihydropyrazolo[1,5-a]pyrazin-5(4H)-yl]but-2-en-1-one

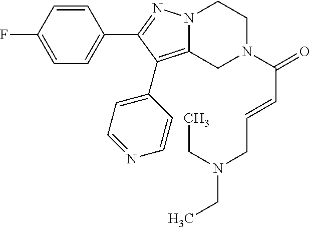 [SMILES: CCN(CC)C/C=C/C(=O)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (60.0 mg, 181 μmol, Intermediate 164) and (2E)-4-(diethylamino)but-2-enoic acid (42.8 mg, 272 μmol) as the starting materials, 49.3 mg (98% purity, 61% yield) of the desired product were prepared after purification by preparative HPLC (basic conditions).

LC-MS (Method 1): Rₜ=0.43 min; MS (ESIpos): m/z=434.1 [M+H]⁺

¹H-NMR (400 MHz, DMSO-d6) delta [ppm]: 0.879 (3.87), 0.948 (6.72), 0.964 (9.48), 2.392 (2.52), 2.449 (5.41), 2.465 (5.01), 2.518 (7.40), 2.523 (5.20), 3.161 (1.73), 3.207 (3.46), 4.071 (1.46), 4.135 (3.04), 4.273 (3.22), 4.820 (4.31), 4.932 (1.56), 6.640 (1.48), 6.715 (1.44), 6.767 (1.73), 6.807 (0.69), 7.153 (7.03), 7.165 (8.05), 7.174 (9.77), 7.197 (16.00), 7.214 (3.08), 7.219 (9.25), 7.352 (4.68), 7.367 (5.94), 7.386 (3.50), 8.539 (7.71), 8.550 (6.96).

### Example 70

### (2E)-1-[2-(4-fluorophenyl)-3-(pyridin-4-yl)-6,7-dihydropyrazolo[1,5-a]pyrazin-5(4H)-yl]-4-hydroxybut-2-en-1-one

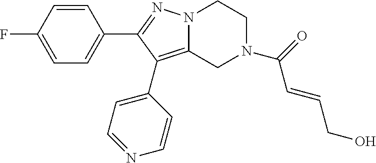 [SMILES: O=C(/C=C/CO)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (60.0 mg, 181 μmol, Intermediate 164) and (2E)-4-hydroxybut-2-enoic acid (27.8 mg, 272 μmol; CAS-RN: [24587-49-3]) as the starting materials, 40.0 mg (98% purity, 57% yield) of the desired product were prepared after purification by preparative HPLC (basic conditions).

LC-MS (Method 1): Rₜ=0.51 min; MS (ESIpos): m/z=379 [M+H]⁺

¹H-NMR (400 MHz, DMSO-d6) δ [ppm]: 2.518 (6.60), 2.523 (4.40), 4.143 (5.54), 4.272 (3.41), 4.830 (4.03), 4.938 (1.12), 5.083 (1.50), 6.570 (0.53), 6.698 (0.82), 6.734 (1.38), 6.793 (3.61), 6.803 (7.87), 6.812 (3.27), 6.831 (1.79), 6.840 (4.43), 6.850 (1.98), 7.158 (6.71), 7.168 (7.99), 7.176 (10.46), 7.198 (15.52), 7.215 (3.06), 7.220 (8.67), 7.355 (4.33), 7.369 (5.53), 7.388 (3.21), 8.541 (16.00), 8.545 (9.96), 8.552 (9.62), 8.556 (14.92).

### Example 71

### (2E)-4-[ethyl(methyl)amino]-1-[2-(4-fluorophenyl)-3-(pyridin-4-yl)-6,7-dihydropyrazolo[1,5-a]pyrazin-5(4H)-yl]but-2-en-1-one

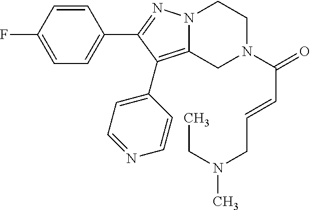 [SMILES: CCN(C)C/C=C/C(=O)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (50.0 mg, 151 μmol, Intermediate 164) and 4-[ethyl(methyl)amino]but-2-enoic acid-hydrogen chloride (1/1) (40.7 mg, 227 μmol) as the starting materials, 40.0 mg (97% purity, 61% yield) of the desired product were prepared after purification by preparative HPLC (basic conditions).

LC-MS (Method 1): Rₜ=0.41 min; MS (ESIpos): m/z=420.5 [M+H]⁺

¹H-NMR (400 MHz, DMSO-d6) δ [ppm]: 0.955 (0.40), 0.974 (0.81), 0.991 (0.96), 1.012 (0.45), 1.154 (4.26), 1.172 (9.31), 1.190 (4.76), 1.231 (0.65), 1.987 (16.00), 2.073 (0.54), 2.119 (0.80), 2.135 (1.48), 2.318 (0.45), 2.322 (0.72), 2.327 (0.94), 2.332 (0.81), 2.352 (0.58), 2.366 (0.46), 2.518 (2.89), 2.523 (1.93), 2.665 (0.48), 2.669 (0.68), 2.673 (0.49), 3.118 (0.75), 3.131 (0.62), 3.999 (1.28), 4.017 (3.82), 4.034 (3.80), 4.052 (1.37), 4.136 (0.56), 4.274 (0.61), 4.821 (0.81), 7.164 (1.48), 7.174 (1.71), 7.186 (0.49), 7.197 (2.72), 7.214 (0.56), 7.219 (1.55), 7.353 (0.87), 7.367 (1.08), 7.383 (0.62), 8.539 (1.55), 8.552 (1.42)

### Example 72

### 1-[2-(4-chloro-2-fluorophenyl)-3-(pyridin-4-yl)-6,7-dihydropyrazolo[1,5-a]pyrazin-5(4H)-yl]-4-(dimethylamino)but-2-yn-1-one

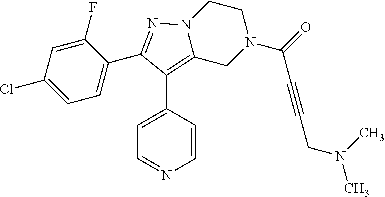 [SMILES: CN(C)CC#CC(=O)N1CCn2nc(-c3ccc(Cl)cc3F)c(-c3ccncc3)c2C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and 4-(dimethylamino)but-2-ynoic acid (20.9 mg, 164 μmol) as the starting materials, 15.7 mg (95% purity, 25% yield) of the desired product were prepared after purification by preparative HPLC (basic conditions).

LC-MS (Method 1): Rₜ=0.54 min; MS (ESIpos): m/z=438 [M+H]⁺

¹H-NMR (400 MHz, DMSO-d6) δ [ppm]: 1.954 (14.67), 2.241 (16.00), 2.523 (0.86), 3.439 (4.35), 3.528 (4.46), 4.099 (0.67), 4.112 (1.22), 4.126 (0.84), 4.255 (0.84), 4.270 (1.29), 4.282 (1.04), 4.292 (1.29), 4.306 (0.96), 4.370 (0.94), 4.384 (1.15), 4.396 (0.51), 4.906 (3.17), 5.206 (3.03), 7.069 (4.35), 7.073 (2.66), 7.080 (2.73), 7.084 (4.38), 7.383 (1.05), 7.404 (1.41), 7.452 (1.63), 7.457 (1.41), 7.478 (1.65), 7.483 (1.44), 7.502 (0.90), 7.522 (1.57), 7.545 (1.40), 7.566 (0.60), 8.488 (2.24), 8.492 (1.36), 8.501 (3.39), 8.512 (1.46), 8.516 (2.19).

### Example 73

### (2E)-1-[2-(4-chloro-2-fluorophenyl)-3-(pyridin-4-yl)-6,7-dihydropyrazolo[1,5-a]pyrazin-5(4H)-yl]-4-fluorobut-2-en-1-one

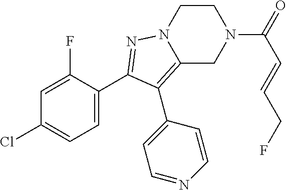 [SMILES: O=C(/C=C/CF)N1CCn2nc(-c3ccc(Cl)cc3F)c(-c3ccncc3)c2C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and (2E)-4-fluorobut-2-enoic acid (17.1 mg, 164 μmol; CAS-RN: [37759-72-1]) as the starting materials, 23.5 mg (96% purity, 40% yield) of the desired product were prepared after purification by preparative HPLC (basic conditions).

LC-MS (Method 1): Rₜ=0.74 min; MS (ESIpos): m/z=414.9 [M+H]⁺

¹H-NMR (400 MHz, DMSO-d6) δ [ppm]: 0.938 (0.84), 0.954 (0.77), 1.232 (0.54), 2.518 (9.31), 2.523 (5.68), 4.107 (2.17), 4.175 (5.68), 4.266 (2.37), 4.316 (5.97), 4.930 (9.09), 5.066 (3.96), 5.090 (4.68), 5.208 (5.07), 6.765 (0.99), 6.801 (4.46), 6.850 (7.97), 6.887 (0.89), 7.084 (11.37), 7.375 (7.24), 7.380 (7.62), 7.396 (9.49), 7.401 (10.47), 7.448 (7.91), 7.453 (7.08), 7.474 (7.86), 7.478 (7.23), 7.498 (4.56), 7.518 (7.14), 7.538 (3.20), 8.504 (16.00), 8.518 (14.80).

### Example 74

### (2E)-1-[2-(4-chloro-2-fluorophenyl)-3-(pyridin-4-yl)-6,7-dihydropyrazolo[1,5-a]pyrazin-5(4H)-yl]-4,4-difluorobut-2-en-1-one

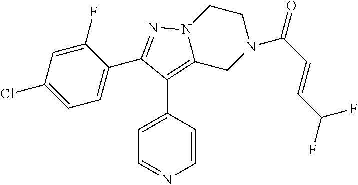 [SMILES: O=C(/C=C/C(F)F)N1CCn2nc(-c3ccc(Cl)cc3F)c(-c3ccncc3)c2C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and (2E)-4,4-difluorobut-2-enoic acid (20.1 mg, 164 μmol; CAS-RN: [37759-73-2]) as the starting materials, 26.5 mg (98% purity, 44% yield) of the desired product were prepared after purification by preparative HPLC (basic conditions).

LC-MS (Method 1): Rₜ=0.79 min; MS (ESIpos): m/z=432.9 [M+H]⁺

¹H-NMR (400 MHz, DMSO-d6) δ [ppm]: 0.935 (1.75), 0.951 (1.68), 1.406 (0.58), 2.518 (5.84), 2.523 (3.77), 4.119 (3.45), 4.132 (2.91), 4.155 (4.30), 4.167 (7.40), 4.181 (4.73), 4.272 (3.38), 4.336 (5.13), 4.349 (7.47), 4.362 (3.74), 4.942 (16.00), 5.081 (6.41), 6.442 (0.55), 6.455 (0.67), 6.476 (1.34), 6.489 (1.48), 6.578 (0.89), 6.591 (2.77), 6.604 (2.00), 6.612 (2.77), 6.622 (8.20), 6.642 (2.23), 6.654 (3.48), 6.667 (2.10), 6.676 (1.08), 6.689 (1.16), 6.718 (0.64), 6.733 (0.57), 6.752 (1.44), 6.765 (1.18), 7.070 (10.44), 7.086 (12.92), 7.104 (4.36), 7.199 (1.37), 7.232 (2.96), 7.242 (3.40), 7.275 (2.36), 7.287 (1.84), 7.377 (5.83), 7.381 (5.47), 7.398 (7.85), 7.402 (7.50), 7.452 (6.39), 7.477 (6.58), 7.481 (5.54), 7.499 (4.68), 7.519 (6.60), 7.540 (2.78), 8.508 (15.41), 8.523 (11.11).

### Example 75

### (2E)-3-chloro-1-[2-(4-chloro-2-fluorophenyl)-3-(pyridin-4-yl)-6,7-dihydropyrazolo[1,5-a]pyrazin-5(4H)-yl]prop-2-en-1-one

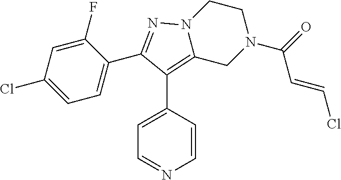 [SMILES: O=C(/C=C/Cl)N1CCn2nc(-c3ccc(Cl)cc3F)c(-c3ccncc3)c2C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and (2E)-3-chloroprop-2-enoic acid (17.5 mg, 164 μmol; CAS-RN: [2345-61-1]) as the starting materials, 23.1 mg (98% purity, 40% yield) of the desired product were prepared after purification by preparative HPLC (basic conditions).

LC-MS (Method 1): Rₜ=0.79 min; MS (ESIpos): m/z=416.9 [M+H]⁺

¹H-NMR (400 MHz, DMSO-d6) δ [ppm]: 2.518 (3.07), 2.523 (2.04), 4.084 (0.89), 4.174 (2.20), 4.258 (1.01), 4.316 (2.35), 4.913 (3.35), 5.077 (1.03), 7.077 (3.54), 7.257 (6.48), 7.289 (12.51), 7.356 (16.00), 7.374 (3.51), 7.379 (3.72), 7.388 (8.15), 7.395 (4.69), 7.400 (5.02), 7.448 (4.43), 7.453 (3.82), 7.472 (4.29), 7.478 (3.95), 7.494 (2.32), 7.514 (3.63), 7.535 (1.63), 8.500 (8.13), 8.515 (7.76).

### Example 76

### [2-(4-chloro-2-fluorophenyl)-3-(pyridin-4-yl)-6,7-dihydropyrazolo[1,5-a]pyrazin-5(4H)-yl](1-methyl-2,5-dihydro-1H-pyrrol-3-yl) methanone

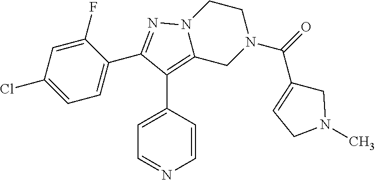 [SMILES: CN1CC=C(C(=O)N2CCn3nc(-c4ccc(Cl)cc4F)c(-c4ccncc4)c3C2)C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and 1-methyl-2,5-dihydro-1H-pyrrole-3-carboxylic acid (20.9 mg, 164 μmol) as the starting materials, 16.5 mg (90% purity, 25% yield) of the desired product were prepared after purification by preparative HPLC (basic conditions).

LC-MS (Method 1): Rₜ=0.50 min; MS (ESIpos): m/z=438 [M+H]⁺

¹H-NMR (400 MHz, DMSO-d6) δ [ppm]: 1.232 (0.63), 2.323 (2.05), 2.327 (2.88), 2.332 (2.52), 2.354 (13.26), 2.518 (6.60), 2.523 (4.38), 2.660 (0.71), 2.665 (1.62), 2.669 (2.21), 2.673 (1.53), 3.375 (0.55), 3.521 (4.86), 3.549 (5.72), 3.634 (1.07), 4.006 (0.62), 4.090 (3.72), 4.104 (6.88), 4.117 (4.64), 4.197 (0.41), 4.211 (0.47), 4.324 (4.17), 4.798 (1.38), 4.909 (3.18), 4.940 (1.88), 6.212 (4.72), 7.060 (12.95), 7.064 (8.27), 7.071 (8.16), 7.075 (14.12), 7.086 (1.00), 7.091 (1.39), 7.095 (0.98), 7.100 (0.56), 7.107 (0.55), 7.110 (0.88), 7.376 (5.23), 7.382 (5.25), 7.397 (6.79), 7.402 (7.09), 7.451 (6.48), 7.456 (5.61), 7.476 (6.37), 7.482 (5.89), 7.496 (6.55), 7.502 (0.98), 7.516 (10.15), 7.537 (4.67), 8.249 (1.13), 8.257 (0.85), 8.492 (1.66), 8.500 (16.00), 8.504 (9.93), 8.511 (9.41), 8.515 (14.96), 10.214 (0.65).

### Example 77

### (2E)-4-(azetidin-1-yl)-1-[2-(4-chloro-2-fluorophenyl)-3-(pyridin-4-yl)-6,7-dihydropyrazolo[1,5-a]pyrazin-5(4H)-yl]but-2-en-1-one

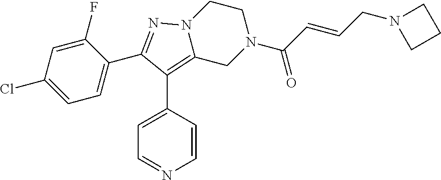 [SMILES: O=C(/C=C/CN1CCC1)N1CCn2nc(-c3ccc(Cl)cc3F)c(-c3ccncc3)c2C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and (2E)-4-(azetidin-1-yl) but-2-enoic acid (23.2 mg, 164 μmol) as the starting materials, 13.2 mg (95% purity, 20% yield) of the desired product were prepared after purification by preparative HPLC (basic conditions).

LC-MS (Method 1): Rₜ=0.52 min; MS (ESIpos): m/z=452 [M+H]⁺

¹H-NMR (400 MHz, DMSO-d6) δ [ppm]: 1.232 (0.56), 1.986 (3.23), 2.518 (10.24), 2.523 (6.05), 3.041 (2.67), 3.143 (12.07), 3.287 (0.56), 3.294 (0.83), 3.300 (0.82), 3.380 (2.21), 3.385 (1.98), 4.144 (4.04), 4.295 (4.28), 4.897 (5.46), 5.011 (1.62), 6.549 (2.06), 6.598 (2.10), 6.644 (2.41), 6.681 (1.05), 7.075 (7.71), 7.194 (0.48), 7.209 (0.54), 7.373 (5.95), 7.378 (6.31), 7.394 (7.79), 7.399 (8.70), 7.446 (7.36), 7.450 (6.55), 7.471 (7.19), 7.476 (6.74), 7.497 (7.72), 7.517 (11.69), 7.537 (5.55), 8.408 (0.48), 8.498 (16.00), 8.502 (11.39), 8.510 (10.69), 8.513 (15.63), 8.535 (0.66), 8.539 (0.92), 8.550 (0.53).

### Example 78

### (2E)-4-fluoro-1-[2-(4-fluorophenyl)-3-(pyridin-4-yl)-6,7-dihydropyrazolo[1,5-a]pyrazin-5(4H)-yl]but-2-en-1-one

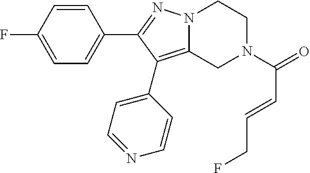 [SMILES: O=C(/C=C/CF)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (60.0 mg, 181 μmol, Intermediate 164) and (2E)-4-fluorobut-2-enoic acid (28.3 mg, 272 μmol; CAS-RN: [37759-72-1]) as the starting materials, 40.0 mg (98% purity, 57% yield) of the desired product were prepared after purification by preparative HPLC (basic conditions).

LC-MS (Method 1): Rₜ=0.61 min; MS (ESIpos): m/z=381 [M+H]⁺

¹H-NMR (400 MHz, DMSO-d6) δ [ppm]: 2.518 (6.92), 2.523 (4.54), 4.096 (1.57), 4.155 (4.18), 4.287 (4.42), 4.842 (6.84), 4.974 (1.79), 5.087 (3.04), 5.206 (3.76), 6.772 (1.94), 6.799 (2.00), 6.844 (5.71), 6.881 (0.63), 7.169 (9.32), 7.176 (11.46), 7.198 (16.00), 7.221 (8.92), 7.358 (5.16), 7.372 (5.83), 8.542 (11.56), 8.556 (10.63).

### Example 79

### (2E)-1-[2-(4-fluorophenyl)-3-(pyridin-4-yl)-6,7-dihydropyrazolo[1,5-a]pyrazin-5(4H)-yl]-4-(pyrrolidin-1-yl) but-2-en-1-one

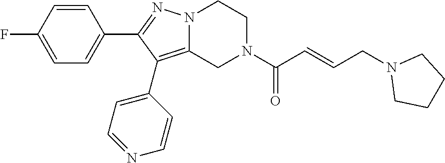 [SMILES: O=C(/C=C/CN1CCCC1)N1CCn2nc(-c3ccc(F)cc3)c(-c3ccncc3)c2C1]

An aliquot of the reaction mixture of crude (2E)-4-bromo-1-[2-(4-fluorophenyl)-3-(pyridin-4-yl)-6,7-dihydropyrazolo[1,5-a]pyrazin-5(4H)-yl]but-2-en-1-one (20.0 mg, 45.3 μmol in DMF, Intermediate 189) was treated with pyrrolidine (34 μl, 680 μmol) and the reaction mixture was stirred at RT for 30 min. Water (100 μl) was added and the reaction mixture was directly purified by reversed phase HPLC (basic conditions) to yield 5.00 mg (95% purity, 24% yield) of the desired product.

LC-MS (Method 1): Rₜ=0.41 min; MS (ESIpos): m/z=432.1 [M+H]⁺

¹H-NMR (400 MHz, DMSO-d6) δ [ppm]: 1.230 (0.86), 1.638 (3.16), 1.687 (7.55), 2.323 (1.25), 2.327 (1.71), 2.332 (1.58), 2.363 (2.63), 2.446 (6.96), 2.518 (4.62), 2.523 (3.14), 2.665 (0.85), 2.669 (1.19), 2.674 (0.84), 3.166 (1.76), 3.214 (4.45), 4.074 (1.53), 4.133 (3.46), 4.273 (3.68), 4.820 (4.89), 4.939 (1.52), 6.626 (0.99), 6.739 (3.16), 7.154 (7.82), 7.166 (9.16), 7.174 (10.31), 7.196 (16.00), 7.219 (9.08), 7.353 (5.33), 7.367 (6.67), 7.387 (4.00), 8.538 (9.21), 8.551 (8.52).

### Example 80

### (2E)-1-[2-(4-fluorophenyl)-3-(pyridin-4-yl)-6,7-dihydropyrazolo[1,5-a]pyrazin-5(4H)-yl]-4-(methylamino)but-2-en-1-one

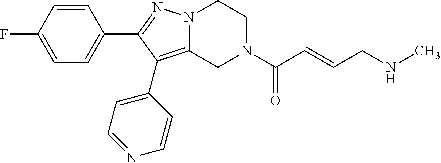 [SMILES: CNC/C=C/C(=O)N1CCn2nc(-c3ccc(F)cc3)c(-c3ccncc3)c2C1]

An aliquot of the reaction mixture of crude (2E)-4-bromo-1-[2-(4-fluorophenyl)-3-(pyridin-4-yl)-6,7-dihydropyrazolo[1,5-a]pyrazin-5(4H)-yl]but-2-en-1-one (60.0 mg, 136 μmol in DMF, Intermediate 189) was treated with methanamine (200 μl, 2.0 M, 410 μmol) and the reaction mixture was stirred at RT for 30 min. Water (100 μl) was added and the reaction mixture was directly purified by reversed phase HPLC (basic conditions) to yield 3 mg (90% purity, 5% yield) of the desired product.

LC-MS (Method 1): Rₜ=0.38 min; MS (ESIpos): m/z=392 [M+H]⁺

¹H-NMR (400 MHz, DMSO-d6) δ [ppm]: 1.231 (1.02), 2.095 (1.21), 2.150 (3.26), 2.216 (1.78), 2.284 (5.67), 2.337 (1.10), 2.518 (10.85), 2.523 (7.39), 2.624 (5.57), 2.635 (5.54), 2.679 (1.15), 3.100 (1.46), 3.145 (1.51), 3.279 (5.46), 3.504 (0.62), 4.012 (0.51), 4.080 (1.55), 4.142 (3.49), 4.271 (3.96), 4.710 (0.42), 4.755 (0.80), 4.824 (4.86), 4.951 (1.27), 6.007 (0.53), 6.046 (0.54), 6.575 (0.70), 6.666 (1.02), 6.722 (4.68), 7.111 (0.44), 7.154 (7.66), 7.167 (9.03), 7.175 (10.54), 7.197 (16.00), 7.220 (8.88), 7.244 (0.42), 7.357 (5.30), 7.368 (6.41), 7.938 (0.71), 8.088 (0.52), 8.327 (0.74), 8.502 (0.54), 8.508 (0.53), 8.517 (0.81), 8.539 (12.12), 8.554 (10.98).

### Example 81

### (2E)-4-(ethylamino)-1-[2-(4-fluorophenyl)-3-(pyridin-4-yl)-6,7-dihydropyrazolo[1,5-a]pyrazin-5(4H)-yl]but-2-en-1-one

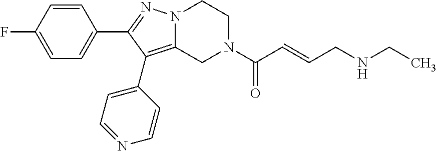 [SMILES: CCNC/C=C/C(=O)N1CCn2nc(-c3ccc(F)cc3)c(-c3ccncc3)c2C1]

An aliquot of the reaction mixture of crude (2E)-4-bromo-1-[2-(4-fluorophenyl)-3-(pyridin-4-yl)-6,7-dihydropyrazolo[1,5-a]pyrazin-5(4H)-yl]but-2-en-1-one (63.8 mg, 145 μmol in DMF, Intermediate 189) was treated with ethanamine (19.5 mg, 434 μmol) and the reaction mixture was stirred at RT for 30 min. Water (100 μl) was added and the reaction mixture was directly purified by reversed phase HPLC (basic conditions) to yield 3 mg (90% purity, 5% yield) of the desired product.

LC-MS (Method 1): Rₜ=0.40 min; MS (ESIpos): m/z=406 [M+H]⁺

¹H-NMR (400 MHz, DMSO-d6) δ [ppm]: 0.851 (0.47), 0.867 (0.55), 0.885 (0.50), 0.904 (0.85), 0.923 (0.87), 1.000 (4.15), 1.018 (7.21), 1.023 (5.16), 1.036 (4.89), 1.160 (0.58), 1.176 (0.59), 1.232 (1.26), 2.518 (13.98), 2.523 (10.16), 2.678 (1.02), 2.962 (0.51), 2.977 (0.78), 2.989 (0.59), 3.012 (0.53), 3.080 (0.43), 3.098 (0.93), 3.112 (0.98), 3.116 (1.07), 3.131 (0.99), 3.148 (0.59), 3.744 (1.60), 4.141 (3.11), 4.208 (1.73), 4.221 (1.80), 4.272 (3.44), 4.823 (4.15), 4.946 (1.09), 6.065 (0.58), 6.104 (0.56), 6.549 (0.56), 6.588 (0.79), 6.602 (0.64), 6.729 (4.76), 7.098 (1.61), 7.102 (1.14), 7.109 (1.11), 7.113 (1.74), 7.165 (8.03), 7.176 (10.15), 7.181 (4.97), 7.187 (3.87), 7.192 (4.81), 7.198 (16.00), 7.203 (4.29), 7.209 (2.26), 7.215 (3.38), 7.220 (8.90), 7.354 (4.81), 7.370 (6.18), 7.384 (3.78), 8.089 (0.87), 8.295 (0.49), 8.493 (1.84), 8.496 (1.00), 8.504 (1.03), 8.508 (1.88), 8.538 (10.90), 8.553 (10.32).

### Example 82

### (2E)-1-[2-(4-fluorophenyl)-3-(pyridin-4-yl)-6,7-dihydropyrazolo[1,5-a]pyrazin-5(4H)-yl]-4-(4-methylpiperazin-1-yl) but-2-en-1-one

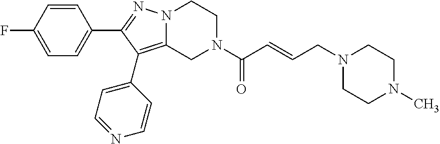 [SMILES: CN1CCN(C/C=C/C(=O)N2CCn3nc(-c4ccc(F)cc4)c(-c4ccncc4)c3C2)CC1]

An aliquot of the reaction mixture of crude (2E)-4-bromo-1-[2-(4-fluorophenyl)-3-(pyridin-4-yl)-6,7-dihydropyrazolo[1,5-a]pyrazin-5(4H)-yl]but-2-en-1-one (63.8 mg, 145 μmol in DMF, Intermediate 189) was treated with 1-methylpiperazine (43.4 mg, 434 μmol) and the reaction mixture was stirred at RT for 30 min. Water (100 μl) was added and the reaction mixture was directly purified by reversed phase HPLC (basic conditions) to yield 3 mg (90% purity, 5% yield) of the desired product.

LC-MS (Method 1): Rₜ=0.41 min; MS (ESIpos): m/z=461.1 [M+H]⁺

¹H-NMR (400 MHz, DMSO-d6) δ [ppm]: 1.229 (0.53), 2.139 (15.60), 2.323 (4.65), 2.327 (4.86), 2.331 (4.60), 2.518 (4.51), 2.523 (3.06), 2.665 (0.73), 2.669 (0.92), 2.673 (0.69), 3.097 (3.56), 4.130 (3.82), 4.274 (3.65), 4.819 (4.44), 4.939 (1.64), 6.633 (2.74), 6.670 (1.60), 6.747 (1.62), 6.785 (0.83), 6.794 (0.98), 6.804 (1.35), 6.813 (0.57), 6.842 (0.67), 7.165 (8.28), 7.173 (11.05), 7.196 (16.00), 7.213 (3.42), 7.218 (9.15), 7.352 (4.97), 7.366 (6.40), 7.386 (3.80), 8.538 (10.76), 8.540 (10.67), 8.552 (10.85).

### Example 83

### (2E)-1-[2-(4-fluorophenyl)-3-(pyridin-4-yl)-6,7-dihydropyrazolo[1,5-a]pyrazin-5(4H)-yl]-4-[(3S)-3-methoxypyrrolidin-1-yl]but-2-en-1-one

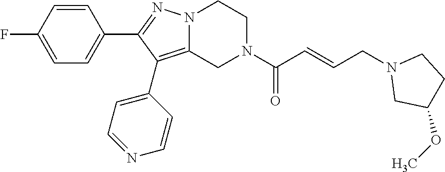 [SMILES: CO[C@H]1CCN(C/C=C/C(=O)N2CCn3nc(-c4ccc(F)cc4)c(-c4ccncc4)c3C2)C1]

An aliquot of the reaction mixture of crude (2E)-4-bromo-1-[2-(4-fluorophenyl)-3-(pyridin-4-yl)-6,7-dihydropyrazolo[1,5-a]pyrazin-5(4H)-yl]but-2-en-1-one (63.8 mg, 145 μmol in DMF, Intermediate 189) was treated with (3S)-3-methoxypyrrolidine-hydrogen chloride (1/1) (59.7 mg, 434 μmol) and the reaction mixture was stirred at RT for 30 min. Water (100 μl) was added and the reaction mixture was directly purified by reversed phase HPLC (basic conditions) to yield 2.3 mg (97% purity, 3% yield) of the desired product.

LC-MS (Method 1): Rₜ=0.48 min; MS (ESIpos): m/z=462 [M+H]⁺

¹H-NMR (400 MHz, DMSO-d6) δ [ppm]: 1.171 (0.45), 1.231 (0.96), 1.636 (1.16), 1.904 (1.03), 1.942 (1.17), 1.959 (1.22), 1.986 (1.43), 2.073 (0.66), 2.083 (6.49), 2.153 (0.65), 2.323 (1.38), 2.327 (1.78), 2.331 (1.45), 2.437 (2.86), 2.518 (4.44), 2.523 (3.04), 2.568 (1.24), 2.665 (1.61), 2.669 (2.17), 2.673 (1.97), 2.689 (1.27), 3.158 (11.42), 3.203 (4.36), 3.215 (4.43), 3.861 (1.55), 4.034 (0.52), 4.080 (1.60), 4.133 (3.36), 4.275 (3.51), 4.820 (4.67), 4.883 (0.54), 4.941 (1.45), 5.756 (0.70), 6.648 (1.51), 6.701 (1.82), 6.737 (1.97), 6.774 (0.67), 7.154 (7.28), 7.165 (8.61), 7.174 (10.44), 7.196 (16.00), 7.213 (3.37), 7.218 (9.12), 7.353 (4.91), 7.367 (6.20), 7.386 (3.68), 8.201 (0.52), 8.538 (9.08), 8.550 (8.65).

### Example 84

### (2E)-4-(3,3-difluoroazetidin-1-yl)-1-[2-(4-fluorophenyl)-3-(pyridin-4-yl)-6,7-dihydropyrazolo[1,5-a]pyrazin-5(4H)-yl]but-2-en-1-one

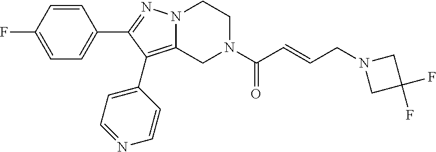 [SMILES: O=C(/C=C/CN1CC(F)(F)C1)N1CCn2nc(-c3ccc(F)cc3)c(-c3ccncc3)c2C1]

An aliquot of the reaction mixture of crude (2E)-4-bromo-1-[2-(4-fluorophenyl)-3-(pyridin-4-yl)-6,7-dihydropyrazolo[1,5-a]pyrazin-5(4H)-yl]but-2-en-1-one (63.8 mg, 145 μmol in DMF, Intermediate 189) was treated with 3,3-difluoroazetidine-hydrogen chloride (1/1) (56.2 mg, 434 μmol) and the reaction mixture was stirred at RT for 30 min. Water (100 μl) was added and the reaction mixture was directly purified by reversed phase HPLC (basic conditions) to yield 2.3 mg 6.10 mg (98% purity, 9% yield) of the desired product.

LC-MS (Method 1): Rₜ=0.58 min; MS (ESIpos): m/z=454 [M+H]⁺

¹H-NMR (400 MHz, DMSO-d6) δ [ppm]: 1.154 (0.68), 1.172 (1.42), 1.190 (0.69), 1.232 (0.59), 1.988 (2.32), 2.084 (1.62), 2.518 (9.44), 2.523 (6.17), 3.359 (4.43), 3.546 (1.74), 3.566 (1.89), 3.623 (3.58), 3.654 (6.06), 3.686 (3.04), 4.017 (0.58), 4.035 (0.77), 4.068 (1.19), 4.142 (3.28), 4.223 (1.32), 4.283 (3.44), 4.818 (5.22), 4.937 (1.29), 6.596 (1.83), 6.634 (1.74), 6.684 (2.03), 6.724 (0.87), 7.161 (5.56), 7.176 (9.29), 7.181 (4.70), 7.198 (16.00), 7.215 (3.08), 7.221 (8.59), 7.354 (4.57), 7.369 (5.75), 7.389 (3.30), 8.538 (8.34), 8.552 (7.34).

### Example 85

### 1-[2-(4-chloro-2-fluorophenyl)-3-(pyridin-4-yl)-6,7-dihydropyrazolo[1,5-a]pyrazin-5(4H)-yl]-4-(morpholin-4-yl) but-2-yn-1-one

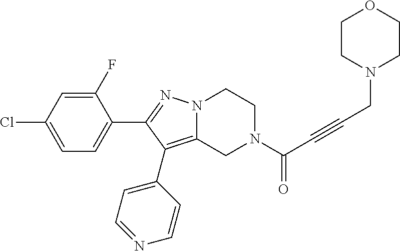 [SMILES: O=C(C#CCN1CCOCC1)N1CCn2nc(-c3ccc(Cl)cc3F)c(-c3ccncc3)c2C1]

Using an analogous method as described for Example 62, 2-(4-chloro-2-fluorophenyl)-3-(pyridin-4-yl)-4,5,6,7-tetrahydropyrazolo[1,5-a]pyrazine-hydrogen chloride (1/1) (50.0 mg, 137 μmol, Intermediate 52) and 4-(morpholin-4-yl) but-2-ynoic acid (27.8 mg, 164 μmol; CAS-RN: [38346-95-1]) as the starting materials, 29.5 mg (99% purity, 44% yield) of the desired product were prepared after purification by preparative HPLC (basic conditions).

LC-MS (Method 1): Rₜ=0.64 min; MS (ESIpos): m/z=480 [M+H]⁺

¹H-NMR (400 MHz, DMSO-d6) δ [ppm]: 2.263 (3.26), 2.274 (4.63), 2.285 (3.55), 2.518 (4.46), 2.523 (2.51), 3.398 (3.87), 3.410 (5.09), 3.421 (3.85), 3.506 (12.59), 3.594 (16.00), 3.599 (5.50), 3.611 (5.99), 3.623 (4.44), 4.091 (1.34), 4.104 (2.71), 4.118 (1.87), 4.255 (1.96), 4.270 (3.98), 4.282 (4.21), 4.295 (2.47), 4.368 (2.39), 4.382 (3.03), 4.394 (1.36), 4.905 (8.36), 5.177 (7.12), 7.069 (10.43), 7.073 (6.37), 7.080 (6.38), 7.084 (10.65), 7.384 (2.61), 7.405 (3.60), 7.452 (4.52), 7.457 (3.87), 7.478 (4.32), 7.483 (3.94), 7.502 (2.47), 7.513 (2.23), 7.522 (3.92), 7.533 (3.42), 7.543 (1.86), 7.554 (1.58), 8.499 (7.32), 8.501 (9.52), 8.505 (4.99), 8.513 (8.22), 8.516 (8.05).

### Example 86

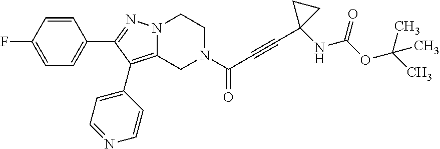 [SMILES: CC(C)(C)OC(=O)NC1(C#CC(=O)N2CCn3nc(-c4ccc(F)cc4)c(-c4ccncc4)c3C2)CC1]

Using an analogous method as described for Example 62 2-(4-fluorophenyl)-3-(pyridin-4-yl)-4,5,6,7-tetrahydropyrazolo[1,5-a]pyrazine-hydrogen chloride (1/1) (50.0 mg, 151 μmol, Intermediate 164) and 3-[1-(tert-butoxycarbonyl) pyrrolidin-2-yl]prop-2-ynoic acid (54.2 mg, 227 μmol; CAS-RN: [1823864-13-6]) as the starting materials, 44.0 mg (99% purity, 56% yield) of the desired product were prepared after purification by preparative HPLC (basic conditions).

LC-MS (Method 1): Rₜ=0.86 min; MS (ESIpos): m/z=502 [M+H]⁺

¹H-NMR (400 MHz, DMSO-d6) delta [ppm]: 1.016 (0.72), 1.024 (1.23), 1.029 (0.92), 1.049 (1.26), 1.099 (0.53), 1.113 (1.46), 1.120 (1.49), 1.131 (0.73), 1.275 (0.93), 1.285 (2.00), 1.293 (1.89), 1.309 (16.00), 1.333 (0.47), 1.346 (0.97), 1.353 (0.91), 1.366 (0.50), 1.411 (13.82), 1.457 (15.06), 2.037 (0.46), 2.050 (0.90), 2.057 (0.91), 2.070 (0.42), 2.518 (2.96), 2.522 (1.86), 4.036 (0.40), 4.050 (0.80), 4.064 (0.56), 4.179 (0.63), 4.193 (1.45), 4.199 (1.38), 4.206 (1.40), 4.227 (0.47), 4.279 (0.91), 4.292 (1.18), 4.786 (3.70), 4.953 (2.05), 6.518 (1.71), 7.143 (3.65), 7.147 (2.65), 7.154 (2.56), 7.159 (4.07), 7.177 (1.60), 7.183 (1.31), 7.188 (0.43), 7.193 (0.70), 7.199 (3.73), 7.205 (2.48), 7.216 (0.61), 7.221 (2.15), 7.227 (1.17), 7.348 (0.38), 7.356 (2.88), 7.362 (1.13), 7.370 (3.12), 7.378 (2.73), 7.386 (0.93), 7.392 (2.32), 7.797 (0.38), 8.531 (3.26), 8.535 (1.95), 8.542 (1.94), 8.546 (3.30), 8.549 (2.01), 8.554 (1.04), 8.561 (0.95), 8.565 (1.47).

### Example 87

### tert-butyl(2RS)-2-(3-[2-(4-fluorophenyl)-3-(pyridin-4-yl)-6,7-dihydropyrazolo[1,5-a]pyrazin-5(4H)-yl]-3-oxoprop-1-yn-1-yl) pyrrolidine-1-carboxylate

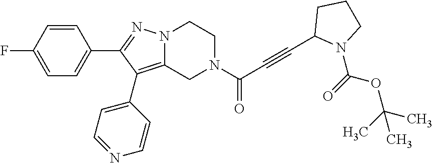 [SMILES: CC(C)(C)OC(=O)N1CCCC1C#CC(=O)N1CCn2nc(-c3ccc(F)cc3)c(-c3ccncc3)c2C1]

Using an analogous method as described for Example 62 2-(4-fluorophenyl)-3-(pyridin-4-yl)-4,5,6,7-tetrahydropyrazolo[1,5-a]pyrazine-hydrogen chloride (1/1) (50.0 mg, 151 μmol, Intermediate 164) and 3-{1-[(tert-butoxycarbonyl)amino]cyclopropyl}prop-2-ynoic acid (51.1 mg, 227 μmol, CAS 1823864-13-6) as the starting materials, 53.5 mg (95% purity, 67% yield) of the desired product were prepared after purification by preparative HPLC (basic conditions).

LC-MS (Method 1): Rₜ=0.94 min; MS (ESIpos): m/z=516.1 [M+H]⁺

¹H-NMR (400 MHz, DMSO-d6) delta [ppm]: 1.317 (2.73), 1.339 (2.77), 1.431 (16.00), 1.735 (0.30), 1.742 (0.28), 1.759 (0.30), 1.766 (0.33), 1.774 (0.29), 1.782 (0.23), 1.916 (0.44), 2.023 (0.32), 2.188 (0.17), 2.518 (1.65), 2.522 (1.04), 3.110 (0.32), 3.121 (0.44), 3.129 (0.35), 3.139 (0.52), 3.159 (0.23), 3.252 (0.27), 3.273 (0.33), 3.293 (0.16), 3.354 (0.32), 4.070 (0.37), 4.085 (0.53), 4.097 (0.31), 4.212 (0.76), 4.226 (1.32), 4.320 (0.52), 4.631 (0.35), 4.802 (1.81), 5.013 (0.94), 7.147 (2.77), 7.151 (1.76), 7.158 (1.86), 7.162 (2.76), 7.178 (1.78), 7.180 (1.66), 7.195 (0.81), 7.200 (3.39), 7.202 (2.68), 7.218 (0.58), 7.223 (1.91), 7.225 (1.40), 7.349 (0.36), 7.357 (2.60), 7.362 (1.02), 7.371 (2.84), 7.379 (2.39), 7.387 (0.81), 7.393 (2.06), 7.400 (0.20), 8.535 (2.88), 8.539 (1.75), 8.542 (2.45), 8.546 (2.60), 8.550 (2.90), 8.553 (1.39), 8.558 (2.00).

### Example 88

### 1-[2-(4-fluorophenyl)-3-(pyridin-4-yl)-6,7-dihydropyrazolo[1,5-a]pyrazin-5(4H)-yl]-3-[(2RS)-pyrrolidin-2-yl]prop-2-yn-1-one

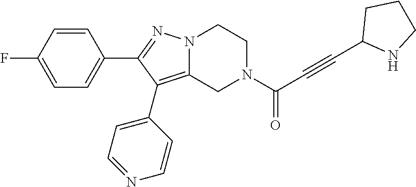 [SMILES: O=C(C#CC1CCCN1)N1CCn2nc(-c3ccc(F)cc3)c(-c3ccncc3)c2C1]

To a solution of tert-butyl(2R)-2-{3-[2-(4-fluorophenyl)-3-(pyridin-4-yl)-6,7-dihydropyrazolo[1,5-a]pyrazin-5(4H)-yl]-3-oxoprop-1-yn-1-yl}pyrrolidine-1-carboxylate (41.2 mg, 79.9 μmol, Example 87) in dichloromethane (2 ml) was added hydrochloric acid (200 μl, 4.0 M, 800 μmol in dioxane) at 0° C. The resulting mixture was stirred at RT for 1 hour. To the reaction mixture was added dichloromethane and sat. sodium hydrogencarbonate solution was added until slight basic pH. After adding some water the mixture was extracted twice with dichloromethane and the organic phase filtered through a hydrophobic filter. 9.50 mg (95% purity, 27% yield) of the desired product were prepared after purification by reversed phase preparative HPLC (basic conditions).

LC-MS (Method 1): Rₜ=0.43 min; MS (ESIpos): m/z=416 [M+H]⁺.

¹H-NMR (400 MHz, DMSO-d6) δ [ppm]: 1.232 (0.57), 1.352 (0.41), 1.461 (0.50), 1.476 (0.60), 1.489 (0.67), 1.544 (0.63), 1.565 (0.64), 1.581 (0.75), 1.600 (0.65), 1.616 (0.64), 1.749 (1.17), 1.767 (1.93), 1.784 (2.09), 1.801 (1.94), 1.847 (1.34), 2.087 (1.08), 2.105 (1.02), 2.518 (16.00), 2.523 (10.74), 2.825 (0.74), 2.865 (0.46), 2.883 (0.74), 3.008 (1.95), 3.024 (3.14), 3.038 (1.74), 3.891 (0.71), 3.988 (2.14), 4.001 (3.60), 4.014 (2.51), 4.057 (1.58), 4.078 (1.75), 4.094 (2.20), 4.111 (1.59), 4.205 (1.99), 4.218 (3.05), 4.232 (1.77), 4.285 (3.06), 4.298 (4.18), 4.311 (2.23), 4.343 (0.67), 4.742 (4.77), 4.785 (0.86), 4.813 (2.02), 4.839 (9.55), 5.039 (1.00), 5.819 (0.41), 6.697 (2.37), 6.828 (3.83), 7.129 (4.27), 7.133 (2.96), 7.141 (3.11), 7.144 (4.76), 7.150 (2.69), 7.154 (2.69), 7.162 (9.45), 7.166 (6.86), 7.174 (7.82), 7.177 (10.16), 7.183 (6.24), 7.188 (3.14), 7.197 (7.23), 7.205 (10.20), 7.219 (3.85), 7.228 (5.67), 7.365 (7.02), 7.379 (8.14), 7.386 (6.21), 7.396 (3.23), 7.401 (4.75), 8.523 (4.38), 8.527 (3.22), 8.539 (6.94), 8.543 (11.42), 8.547 (6.61), 8.555 (7.34), 8.558 (8.97).

### Example 89

### 3-(1-aminocyclopropyl)-1-[2-(4-fluorophenyl)-3-(pyridin-4-yl)-6,7-dihydropyrazolo[1,5-a]pyrazin-5(4H)-yl]prop-2-yn-1-one

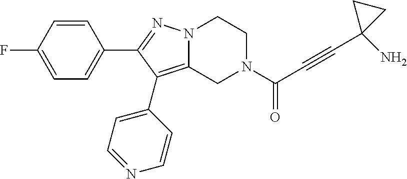 [SMILES: NC1(C#CC(=O)N2CCn3nc(-c4ccc(F)cc4)c(-c4ccncc4)c3C2)CC1]

Using an analogous method as described for Example 88, tert-butyl (1-{3-[2-(4-fluorophenyl)-3-(pyridin-4-yl)-6,7-dihydropyrazolo[1,5-a]pyrazin-5(4H)-yl]-3-oxoprop-1-yn-1-yl}cyclopropyl) carbamate (50.8 mg, 101 μmol, Example 86) was used as the starting material. 25.0 mg (97% purity, 60% yield) of the desired product were prepared after purification by preparative HPLC (basic conditions).

LC-MS (Method 1): Rₜ=0.43 min; MS (ESIpos): m/z=402 [M+H]⁺

¹H-NMR (400 MHz, DMSO-d6) δ [ppm]: 0.749 (0.54), 0.762 (1.65), 0.769 (2.35), 0.777 (1.80), 0.790 (0.75), 0.826 (1.18), 0.835 (2.20), 0.842 (1.87), 0.856 (3.33), 0.862 (2.89), 0.872 (1.29), 0.909 (1.97), 0.921 (3.16), 0.928 (3.66), 0.937 (1.67), 1.044 (1.77), 1.052 (3.84), 1.060 (3.01), 1.071 (1.22), 1.085 (1.29), 1.095 (3.05), 1.102 (2.83), 1.113 (1.02), 1.233 (0.46), 2.318 (1.32), 2.518 (16.00), 2.523 (10.96), 2.678 (1.32), 4.023 (1.12), 4.036 (2.18), 4.050 (1.73), 4.071 (1.03), 4.091 (0.83), 4.211 (2.53), 4.222 (3.01), 4.235 (1.99), 4.264 (1.49), 4.278 (2.28), 4.294 (2.48), 4.308 (2.15), 4.771 (1.91), 4.789 (6.19), 4.833 (5.12), 4.964 (3.19), 6.543 (0.40), 6.650 (1.54), 6.759 (4.62), 7.136 (1.37), 7.145 (5.72), 7.150 (4.37), 7.157 (3.89), 7.161 (8.48), 7.167 (4.33), 7.172 (4.44), 7.179 (6.00), 7.183 (5.17), 7.202 (6.77), 7.204 (5.49), 7.207 (4.68), 7.224 (4.02), 7.230 (2.08), 7.360 (3.40), 7.367 (3.51), 7.374 (5.01), 7.381 (5.40), 7.389 (3.96), 7.396 (3.58), 7.403 (2.16), 7.410 (1.33), 8.533 (8.70), 8.537 (5.68), 8.540 (5.76), 8.544 (7.69), 8.548 (8.37), 8.555 (3.85).

### Example 90

### tert-butyl {4-[2-(4-fluorophenyl)-3-(pyridin-4-yl)-6,7-dihydropyrazolo[1,5-a]pyrazin-5(4H)-yl]-4-oxobut-2-yn-1-yl}carbamate

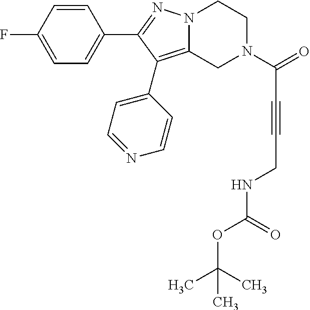 [SMILES: CC(C)(C)OC(=O)NCC#CC(=O)N1CCn2nc(-c3ccc(F)cc3)c(-c3ccncc3)c2C1]

Using an analogous method as described for Example 62, 2-(4-fluorophenyl)-3-(pyridin-4-yl)-4,5,6,7-tetrahydropyrazolo[1,5-a]pyrazine-hydrogen chloride (1/1) (100 mg, 302 μmol, Intermediate 164) and 4-[(tert-butoxycarbonyl)amino]but-2-ynoic acid (72.3 mg, 363 μmol; CAS-RN: [168762-94-5]) as the starting materials, 131 mg (99% purity, 90% yield) of the desired product were prepared after purification by preparative HPLC (acidic conditions).

LC-MS (Method 1): Rₜ=0.81 min; MS (ESIpos): m/z=476 [M+H]⁺

¹H-NMR (400 MHz, DMSO-d6) δ [ppm]: 1.341 (4.00), 1.388 (1.38), 1.409 (16.00), 2.518 (1.54), 2.523 (1.07), 3.916 (0.44), 3.979 (1.03), 3.993 (1.00), 4.232 (0.48), 4.244 (0.53), 4.254 (0.84), 4.268 (0.77), 4.304 (0.73), 4.317 (0.77), 4.804 (2.40), 5.038 (1.10), 7.155 (1.77), 7.159 (1.26), 7.166 (1.46), 7.170 (2.03), 7.179 (1.90), 7.184 (0.57), 7.195 (0.61), 7.201 (3.18), 7.207 (0.58), 7.218 (0.53), 7.224 (1.72), 7.338 (0.49), 7.352 (0.66), 7.359 (1.47), 7.364 (0.53), 7.373 (1.50), 7.381 (1.14), 7.390 (0.41), 7.395 (0.99), 8.538 (1.12), 8.553 (1.49), 8.568 (0.50).

### Example 91

### 4-amino-1-[2-(4-fluorophenyl)-3-(pyridin-4-yl)-6,7-dihydropyrazolo[1,5-a]pyrazin-5(4H)-yl]but-2-yn-1-one

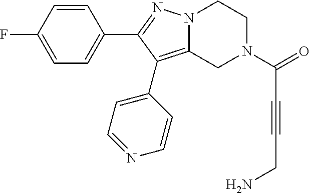 [SMILES: NCC#CC(=O)N1CCn2nc(-c3ccc(F)cc3)c(-c3ccncc3)c2C1]

Using an analogous method as described for Example 88, tert-butyl {4-[2-(4-fluorophenyl)-3-(pyridin-4-yl)-6,7-dihydropyrazolo[1,5-a]pyrazin-5(4H)-yl]-4-oxobut-2-yn-1-yl}carbamate (124 mg, 261 μmol, Example 90) was used as the starting material. 20.0 mg (90% purity, 18% yield) of the desired product were prepared after purification by preparative HPLC (basic conditions).

LC-MS (Method 1): Rₜ=0.81 min; MS (ESIpos): m/z=376 [M+H]⁺

¹H-NMR (400 MHz, DMSO-d6) δ [ppm]: 0.866 (0.50), 1.231 (0.98), 1.341 (3.10), 1.409 (11.52), 2.518 (16.00), 2.523 (10.03), 2.692 (0.73), 3.394 (1.30), 3.930 (0.75), 3.979 (1.43), 3.993 (1.46), 4.056 (1.10), 4.230 (1.61), 4.254 (1.80), 4.266 (1.73), 4.304 (1.50), 4.802 (2.43), 5.037 (0.97), 6.547 (0.51), 7.148 (4.56), 7.152 (4.21), 7.159 (4.72), 7.163 (5.40), 7.177 (5.32), 7.200 (6.18), 7.222 (3.24), 7.358 (3.64), 7.372 (3.54), 7.380 (2.96), 7.394 (2.05), 7.490 (0.44), 8.534 (3.64), 8.538 (2.97), 8.545 (2.84), 8.549 (3.65), 8.565 (2.00).

### Example 92

### 1-[2-(4-chloro-2-fluoroanilino)-3-(3-methyl-1H-pyrrolo[2,3-b]pyridin-4-yl)-6,7-dihydropyrazolo[1,5-a]pyrazin-5(4H)-yl]prop-2-en-1-one

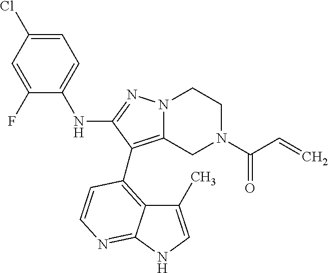 [SMILES: C=CC(=O)N1CCn2nc(Nc3ccc(Cl)cc3F)c(-c3ccnc4[nH]cc(C)c34)c2C1]

To a stirred solution of N-(4-chloro-2-fluorophenyl)-3-(3-methyl-1H-pyrrolo[2,3-b]pyridin-4-yl)-4,5,6,7-tetrahydropyrazolo[1,5-a]pyrazin-2-amine-hydrogen chloride (1/1) (67.6 mg, 156 μmol, Intermediate 157) in DMF (1.9 ml), N,N-diisopropylethylamine (160 μl, 940 μmol), prop-2-enoic acid (13.5 mg, 187 μmol; CAS-RN: [79-10-7]) finally 2,4,6-tripropyl-1,3,5,2lambda5,4lambda5,6lambda5-trioxatriphosphinane-2,4,6-trione (140 μl, 50% purity in DMF, 230 μmol; CAS-RN: [68957-94-8]) were added at rt. After stirring for 30 min at RT, water was added and the mixture was extracted with ethyl acetate twice. The combined organic layers were concentrated under reduced pressure and the residue was purified by reversed phase column chromatography (Biotage Sfär C18 (A30 μm), basic conditions) yielding 36.6 mg (98% purity, 51% yield) of the title compound.

LC-MS (Method 1): Rₜ=0.99 min; MS (ESIpos): m/z=451 [M+H]⁺

¹H-NMR (400 MHz, DMSO-d6) δ [ppm]: 1.937 (16.00), 2.075 (1.39), 2.518 (4.31), 2.523 (3.02), 4.052 (1.14), 4.076 (1.11), 4.143 (1.53), 4.192 (4.92), 4.464 (1.06), 4.506 (1.90), 4.586 (3.11), 4.627 (1.99), 4.721 (0.50), 5.682 (0.44), 5.760 (1.36), 5.787 (1.35), 6.104 (0.44), 6.148 (1.77), 6.190 (1.44), 6.843 (2.21), 6.855 (2.44), 6.917 (1.03), 6.944 (0.98), 6.958 (0.92), 6.984 (0.78), 7.053 (2.88), 7.056 (3.13), 7.075 (3.13), 7.078 (3.45), 7.101 (0.69), 7.213 (8.58), 7.219 (11.63), 7.242 (4.87), 7.248 (4.70), 7.665 (1.21), 7.688 (2.36), 7.710 (1.49), 8.167 (11.33), 8.179 (10.54), 11.431 (2.87).

### Example 93

### 1-[2-(3-fluoro-4-methoxyanilino)-3-(3-methyl-1H-pyrrolo[2,3-b]pyridin-4-yl)-6,7-dihydropyrazolo[1,5-a]pyrazin-5(4H)-yl]prop-2-en-1-one

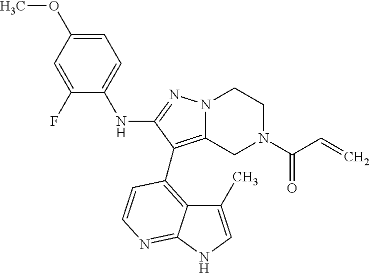 [SMILES: C=CC(=O)N1CCn2nc(Nc3ccc(OC)cc3F)c(-c3ccnc4[nH]cc(C)c34)c2C1]

Using an analogous method as described for Example 92, N-(3-fluoro-4-methoxyphenyl)-3-(3-methyl-1H-pyrrolo[2,3-b]pyridin-4-yl)-4,5,6,7-tetrahydropyrazolo[1,5-a]pyrazin-2-amine-hydrogen chloride (1/1) (49.8 mg, 116 μmol, Intermediate 158) was used as the starting material. 21.3 mg (95% purity, 39% yield) of the desired product were prepared after purification by preparative HPLC (basic conditions).

LC-MS (Method 1): Rₜ=0.8 min; MS (ESIpos): m/z=447 [M+H]⁺

¹H-NMR (400 MHz, DMSO-d6) δ [ppm]: 1.907 (4.61), 2.518 (1.07), 2.523 (0.67), 3.717 (16.00), 4.109 (0.48), 4.164 (1.48), 4.472 (0.78), 4.496 (0.91), 5.751 (0.43), 5.777 (0.43), 6.136 (0.49), 6.178 (0.45), 6.819 (0.69), 6.831 (0.76), 6.905 (1.03), 6.927 (1.65), 6.939 (0.44), 6.951 (1.39), 7.089 (0.98), 7.092 (0.98), 7.111 (0.75), 7.114 (0.76), 7.182 (1.45), 7.397 (0.60), 7.434 (0.58), 7.661 (0.88), 8.179 (2.59), 8.191 (2.45), 11.376 (0.84).

### Example 94

### 1-[3-(3-methyl-1H-pyrrolo[2,3-b]pyridin-4-yl)-2-[4-(trifluoromethyl) anilino]-6,7-dihydropyrazolo[1,5-a]pyrazin-5(4H)-yl]prop-2-en-1-one

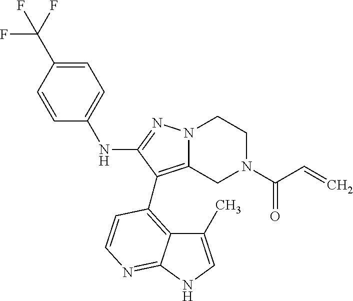 [SMILES: C=CC(=O)N1CCn2nc(Nc3ccc(C(F)(F)F)cc3)c(-c3ccnc4[nH]cc(C)c34)c2C1]

Using an analogous method as described for Example 92, 3-(3-methyl-1H-pyrrolo[2,3-b]pyridin-4-yl)-N-[4-(trifluoromethyl)phenyl]-4,5,6,7-tetrahydropyrazolo[1,5-a]pyrazin-2-amine-hydrogen chloride (1/1) (36.8 mg, 82.0 μmol, Intermediate 159) was used as the starting material. 17.6 mg (95% purity, 44% yield) of the desired product were prepared after purification by preparative HPLC (basic conditions).

LC-MS (Method 1): Rₜ=1.02 min; MS (ESIpos): m/z=467 [M+H]⁺

¹H-NMR (400 MHz, DMSO-d6) δ [ppm]: 0.940 (0.56), 0.956 (0.55), 1.232 (0.91), 1.894 (16.00), 2.518 (5.94), 2.523 (4.05), 4.061 (1.51), 4.080 (1.33), 4.097 (1.00), 4.143 (1.62), 4.199 (4.89), 4.449 (0.87), 4.491 (2.48), 4.528 (3.27), 4.570 (1.36), 4.693 (0.51), 4.733 (0.43), 5.674 (0.50), 5.758 (1.63), 5.784 (1.66), 6.145 (1.80), 6.185 (1.63), 6.834 (2.48), 6.846 (2.75), 6.917 (1.11), 6.943 (1.16), 6.958 (1.04), 6.984 (0.89), 7.186 (5.06), 7.414 (6.19), 7.436 (13.06), 7.472 (10.57), 7.494 (5.40), 8.183 (10.05), 8.195 (9.29), 8.236 (0.87), 8.301 (3.41), 11.394 (3.01).

### Example 95

### 1-[2-(4-chloro-2-fluoroanilino)-3-(pyridin-4-yl)-6,7-dihydropyrazolo[1,5-a]pyrazin-5(4H)-yl]prop-2-en-1-one

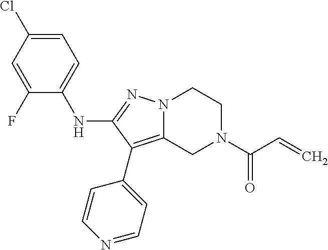 [SMILES: C=CC(=O)N1CCn2nc(Nc3ccc(Cl)cc3F)c(-c3ccncc3)c2C1]

Using an analogous method as described for Example 92, N-(4-chloro-2-fluorophenyl)-3-(pyridin-4-yl)-4,5,6,7-tetrahydropyrazolo[1,5-a]pyrazin-2-amine-hydrogen chloride (1/1) (60.1 mg, 158 μmol. Intermediate 160) was used as the starting material. 26.2 mg (98% purity, 41% yield) of the desired product were prepared after purification by preparative HPLC (basic conditions).

LC-MS (Method 1): Rₜ=0.71 min; MS (ESIpos): m/z=398 [M+H]⁺

¹H-NMR (400 MHz, DMSO-d6) δ [ppm]: 0.940 (0.48), 0.956 (0.45), 1.232 (0.44), 2.148 (0.46), 2.518 (4.95), 2.523 (3.34), 4.072 (2.43), 4.149 (16.00), 4.335 (0.42), 4.854 (0.57), 4.909 (8.16), 5.035 (2.59), 5.772 (2.63), 5.797 (2.24), 6.160 (4.90), 6.166 (4.75), 6.202 (5.38), 6.207 (5.30), 6.922 (2.12), 6.948 (2.21), 6.963 (1.97), 6.993 (5.70), 6.998 (5.50), 7.015 (6.15), 7.017 (6.24), 7.020 (6.91), 7.022 (6.01), 7.137 (2.85), 7.158 (3.74), 7.180 (1.64), 7.288 (9.39), 7.293 (8.89), 7.316 (9.16), 7.322 (8.89), 7.371 (6.69), 7.384 (8.09), 7.929 (5.45), 8.539 (10.55).

### Example 96

### 1-[2-(3-fluoro-4-methoxyanilino)-3-(pyridin-4-yl)-6,7-dihydropyrazolo[1,5-a]pyrazin-5(4H)-yl]prop-2-en-1-one

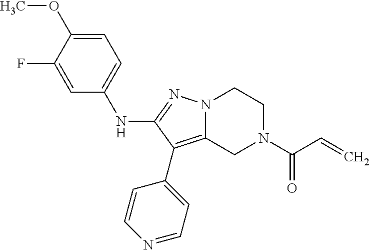 [SMILES: C=CC(=O)N1CCn2nc(Nc3ccc(OC)c(F)c3)c(-c3ccncc3)c2C1]

Using an analogous method as described for Example 92, N-(3-fluoro-4-methoxyphenyl)-3-(pyridin-4-yl)-4,5,6,7-tetrahydropyrazolo[1,5-a]pyrazin-2-amine-hydrogen chloride (1/1) (47.0 mg, 125 μmol, Intermediate 161) was used as the starting material. 32.6 mg (97% purity, 64% yield) of the desired product were prepared after purification by preparative HPLC (basic conditions).

LC-MS (Method 1): Rₜ=0.58 min; MS (ESIpos): m/z=394 [M+H]⁺

¹H-NMR (400 MHz, DMSO-d6) delta [ppm]: 1.752 (0.41), 2.518 (0.71), 2.523 (0.47), 3.729 (16.00), 3.737 (1.14), 4.075 (0.41), 4.129 (2.26), 4.857 (1.12), 4.978 (0.33), 5.751 (0.23), 5.766 (0.35), 5.782 (0.23), 5.793 (0.31), 6.152 (0.59), 6.156 (0.58), 6.193 (0.65), 6.198 (0.63), 6.918 (0.26), 6.938 (0.37), 6.946 (0.35), 6.960 (1.77), 6.974 (1.99), 7.173 (0.40), 7.209 (0.36), 7.355 (0.91), 7.367 (1.15), 8.027 (0.71), 8.558 (1.35).

### Example 97

### 1-[3-(pyridin-4-yl)-2-[4-(trifluoromethyl) anilino]-6,7-dihydropyrazolo[1,5-a]pyrazin-5(4H)-yl]prop-2-en-1-one

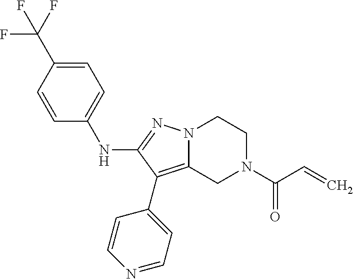 [SMILES: C=CC(=O)N1CCn2nc(Nc3ccc(C(F)(F)F)cc3)c(-c3ccncc3)c2C1]

Using an analogous method as described for Example 92, 3-(pyridin-4-yl)-N-[4-(trifluoromethyl)phenyl]-4,5,6,7-tetrahydropyrazolo[1,5-a]pyrazin-2-amine-hydrogen chloride (1/1) (62.1 mg, 157 μmol, Intermediate 162) was used as the starting material. 25.6 mg (94% purity, 37% yield) of the desired product were prepared after purification by preparative HPLC (basic conditions).

LC-MS (Method 1): Rₜ=0.75 min; MS (ESIpos): m/z=414 [M+H]⁺

¹H-NMR (400 MHz, DMSO-d6) δ [ppm]: 1.231 (0.45), 1.751 (3.70), 2.337 (0.46), 2.518 (4.43), 2.523 (2.96), 2.679 (0.48), 2.933 (0.46), 4.078 (2.04), 4.164 (9.24), 4.336 (0.63), 4.844 (0.83), 4.900 (7.30), 5.024 (2.27), 5.774 (2.31), 5.801 (2.01), 6.162 (4.36), 6.167 (4.21), 6.184 (0.43), 6.204 (4.72), 6.208 (4.66), 6.926 (1.88), 6.953 (1.87), 6.967 (1.54), 6.993 (1.13), 7.237 (7.14), 7.257 (7.53), 7.359 (6.19), 7.371 (7.59), 7.443 (16.00), 7.464 (13.03), 8.561 (9.12), 8.628 (5.44).

### Example 98

### (2E)-4-(dimethylamino)-1-[2-(4-fluorophenyl)-3-(1H-pyrrolo[2,3-b]pyridin-4-yl)-6,7-dihydropyrazolo[1,5-a]pyrazin-5(4H)-yl]but-2-en-1-one

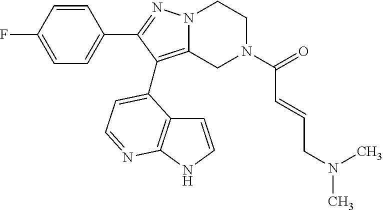 [SMILES: CN(C)C/C=C/C(=O)N1CCn2nc(-c3ccc(F)cc3)c(-c3ccnc4[nH]ccc34)c2C1]

2-(4-fluorophenyl)-3-(1H-pyrrolo[2,3-b]pyridin-4-yl)-4,5,6,7-tetrahydropyrazolo[1,5-a]pyrazine-hydrogen chloride (51.6 mg, 139 μmol, Intermediate 165) was dissolved in DMF (1 ml), then N,N-diisopropylethylamine (160 μl, 930 μmol; CAS-RN: [7087-68-5]), T3P (140 μl, 50% purity in DMF, 230 μmol; CAS-RN: [68957-94-8]) and (2E)-4-(dimethylamino)but-2-enoic acid (24.0 mg, 186 μmol) were added, the mixture was stirred at RT over night. The reaction was quenched with water (0.1 ml) and the mixture purified by reverse phase C18 HPLC yielding the title compound (6.00 mg, 90% purity, 8% yield).

LC-MS (Method 3): Rₜ=0.67 min; MS (ESIpos): m/z=443.2 [M+H]⁺

¹H-NMR (400 MHz, DMSO-d6) delta [ppm]: 0.899 (0.80), 0.905 (0.84), 0.917 (1.92), 0.932 (1.54), 0.936 (1.59), 0.953 (0.51), 1.475 (1.87), 2.016 (0.94), 2.097 (1.36), 2.202 (4.30), 2.337 (0.94), 2.518 (16.00), 2.523 (10.43), 2.679 (0.98), 3.118 (1.12), 3.425 (7.20), 3.472 (3.23), 3.485 (2.90), 3.497 (1.78), 3.500 (1.68), 3.601 (0.51), 4.114 (0.80), 4.168 (1.22), 4.319 (1.36), 4.664 (2.15), 4.764 (0.65), 5.964 (2.29), 5.968 (2.71), 5.972 (2.71), 5.977 (2.29), 6.660 (0.51), 6.758 (0.42), 6.884 (1.31), 7.041 (2.20), 7.064 (4.26), 7.085 (2.34), 7.200 (0.42), 7.222 (0.80), 7.244 (0.42), 7.321 (2.39), 7.379 (2.11), 8.089 (0.42), 8.212 (4.91), 8.224 (4.54), 11.721 (1.64).

### Example 99

### (2E)-4-(dimethylamino)-1-[2-(2-fluoro-4-methylphenyl)-3-(1H-pyrrolo[2,3-b]pyridin-4-yl)-6,7-dihydropyrazolo[1,5-a]pyrazin-5(4H)-yl]but-2-en-1-one

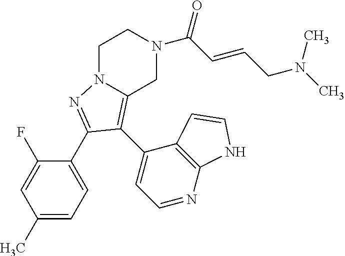 [SMILES: Cc1ccc(-c2nn3c(c2-c2ccnc4[nH]ccc24)CN(C(=O)/C=C/CN(C)C)CC3)c(F)c1]

2-(2-fluoro-4-methylphenyl)-3-(1H-pyrrolo[2,3-b]pyridin-4-yl)-4,5,6,7-tetrahydropyrazolo[1,5-a]pyrazine-hydrogen chloride (1/2) (125 mg, 297 μmol, Intermediate 166) was dissolved in DMF (850 μl), then N,N-diisopropylethylamine (310 μl, 1.8 mmol; CAS-RN: [7087-68-5]), (2E)-4-(dimethylamino)but-2-enoic acid-hydrogen chloride (1/1) (54.2 mg, 327 μmol) and T3P (260 μl, 50% purity in DMF, 450 μmol CAS-RN: [68957-94-8]) were added, stirred at RT for 30 minutes. Water was added and the pH adjusted to 10 using sodium hydroxide solution, and the aqueous layer was extracted with ethyl acetate (2×). Purification by basic reverse phase prep HPLC C18, yielded the title compound (39.0 mg, 95% purity, 27% yield)

LC-MS (Method 2): Rₜ=0.95 min; MS (ESIpos): m/z=459 [M+H]⁺

¹H-NMR (400 MHz, DMSO-d6) delta [ppm]: 2.045 (1.92), 2.075 (1.39), 2.157 (8.56), 2.267 (16.00), 2.318 (0.71), 2.518 (8.08), 2.523 (5.67), 2.660 (0.68), 2.962 (0.49), 3.053 (1.62), 3.066 (1.50), 4.124 (0.53), 4.185 (1.54), 4.326 (1.62), 4.741 (2.44), 4.842 (0.60), 6.000 (1.84), 6.630 (0.53), 6.668 (0.68), 6.732 (1.58), 6.777 (0.68), 6.851 (1.92), 6.879 (1.88), 6.965 (1.77), 6.985 (1.92), 7.276 (0.98), 7.295 (1.54), 7.316 (1.13), 7.335 (1.88), 8.121 (6.80), 8.133 (6.31), 11.638 (1.65).

Analogously to Example 99 the following examples were prepared from the states starting material, (2E)-4-(dimethylamino)but-2-enoic acid-hydrogen chloride (1/1), in combination with T3P and DIPEA in DMF.

| Example 100 | 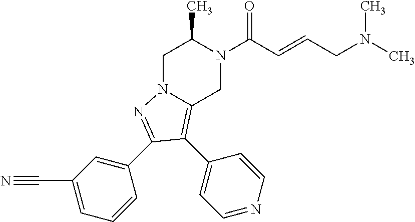 [SMILES: C[C@@H]1Cn2nc(-c3cccc(C#N)c3)c(-c3ccncc3)c2CN1C(=O)/C=C/CN(C)C] |
|---|---|
| | 3-[(6R)-5-[(2E)-4-(dimethylamino)but-2-enoyl]-6-methyl-3-(pyridin-4- |
| | yl)-4,5,6,7-tetrahydropyrazolo[1,5-a]pyrazin-2-yl]benzonitrile |
| | Produced from 3-[(6R)-6-methyl-3-(pyridin-4-yl)-4,5,6,7- |
| | tetrahydropyrazolo[1,5-a]pyrazin-2-yl]benzonitrile hydrochloride (1:1) |
| | (Intermediate 187) |
| | LC-MS (Method 2): Rₜ = 0.93 min; MS (ESlpos): m/z = 427 [M + H]⁺ |
| | 1H-NMR (400 MHz, DMSO-d6) delta [ppm]: 1.234 (3.59), 2.142 (16.00), |
| | 2.518 (7.14), 2.523 (4.85), 2.659 (0.65), 3.039 (5.33), 4.246 (1.97), 4.277 |
| | (3.43), 4.356 (0.74), 4.917 (0.52), 5.114 (1.71), 5.159 (1.39), 6.641 |
| | (1.45), 6.661 (1.97), 6.676 (2.33), 7.250 (8.79), 7.264 (8.89), 7.547 |
| | (3.01), 7.566 (7.56), 7.585 (5.56), 7.627 (3.39), 7.630 (5.53), 7.634 |
| | (4.01), 7.646 (2.20), 7.650 (3.30), 7.653 (2.17), 7.767 (5.75), 7.802 |
| | (4.43), 7.806 (5.88), 7.809 (3.46), 7.821 (3.75), 7.825 (5.17), 7.828 |
| | (3.01), 8.569 (10.63), 8.572 (7.08), 8.580 (6.76), 8.584 (10.02). |
| Example 101 | 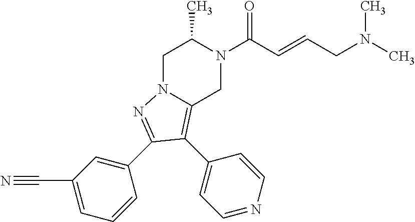 [SMILES: C[C@H]1Cn2nc(-c3cccc(C#N)c3)c(-c3ccncc3)c2CN1C(=O)/C=C/CN(C)C] |
| | 3-[(6S)-5-[(2E)-4-(dimethylamino)but-2-enoyl]-6-methyl-3-(pyridin-4- |
| | yl)-4,5,6,7-tetrahydropyrazolo[1,5-a]pyrazin-2-yl]benzonitrile |
| | Produced from 3-[(6S)-6-methyl-3-(pyridin-4-yl)-4,5,6,7- |
| | tetrahydropyrazolo[1,5-a]pyrazin-2-yl]benzonitrile hydrochloride (1:1) |
| | (Intermediate 188). |
| | LC-MS (Method 2): Rₜ = 0.93 min; MS (ESlpos): m/z = 427 [M + H]⁺ |
| | 1H-NMR (400 MHz, DMSO-d6) delta [ppm]: 1.233 (3.75), 1.352 (0.58), |
| | 1.905 (0.52), 2.058 (0.43), 2.145 (16.00), 2.337 (0.61), 2.518 (5.09), |
| | 2.523 (3.44), 2.679 (0.58), 3.044 (5.61), 4.247 (1.98), 4.278 (3.47), 4.356 |
| | (0.76), 4.924 (0.55), 5.116 (1.71), 5.158 (1.37), 6.642 (1.49), 6.661 |
| | (1.98), 6.677 (2.35), 6.691 (1.34), 7.249 (8.93), 7.264 (9.11), 7.547 |
| | (3.20), 7.566 (8.05), 7.585 (5.91), 7.627 (3.66), 7.630 (5.82), 7.634 |
| | (4.14), 7.646 (2.32), 7.651 (3.44), 7.654 (2.22), 7.767 (5.94), 7.802 |
| | (5.09), 7.806 (6.46), 7.810 (3.84), 7.821 (4.33), 7.825 (5.61), 7.829 |
| | (3.35), 8.187 (2.74), 8.568 (10.48), 8.572 (7.01), 8.580 (6.83), 8.583 |
| | (10.00). |

### Example 102

### 1-[2-(2-fluoro-4-methylphenyl)-3-(1H-pyrrolo[2,3-b]pyridin-4-yl)-6,7-dihydropyrazolo[1,5-a]pyrazin-5(4H)-yl]prop-2-en-1-one

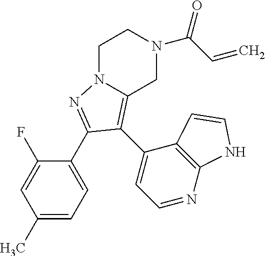 [SMILES: C=CC(=O)N1CCn2nc(-c3ccc(C)cc3F)c(-c3ccnc4[nH]ccc34)c2C1]

2-(2-fluoro-4-methylphenyl)-3-(1H-pyrrolo[2,3-b]pyridin-4-yl)-4,5,6,7-tetrahydropyrazolo[1,5-a]pyrazine-hydrogen chloride (1/2) (125 mg, 297 μmol, Intermediate 166) was dissolved in DMF (850 μl), then N, N-diisopropylethylamine (310 μl, 1.8 mmol; CAS-RN: [7087-68-5]), prop-2-enoic acid (22 μl, 330 μmol) and T3P (260 μl, 50% purity in DMF, 450 μmol CAS-RN: [68957-94-8]) were added, stirred at RT for 30 minutes Water was added and the pH adjusted to 10 using sodium hydroxide solution, and the aqueous layer was extracted with ethyl acetate (2×). Purification by basic reverse phase prep HPLC C18, yielded the title compound (35.0 mg, 95% purity, 28% yield)

LC-MS (Method 2): Rₜ=0.95 min; MS (ESIpos): m/z=402 [M+H]⁺

¹H-NMR (400 MHz, DMSO-d6) ¿ [ppm]: 2.075 (2.08), 2.268 (11.45), 2.318 (1.24), 2.518 (16.00), 2.523 (11.05), 4.133 (0.46), 4.205 (1.37), 4.333 (1.48), 4.756 (2.34), 4.856 (0.56), 5.761 (0.66), 5.787 (0.66), 6.008 (1.13), 6.143 (0.74), 6.185 (0.71), 6.723 (1.06), 6.735 (1.08), 6.852 (1.35), 6.880 (1.30), 6.921 (0.49), 6.967 (1.59), 6.988 (1.78), 7.280 (0.78), 7.299 (1.44), 7.319 (1.06), 7.339 (1.32), 8.124 (4.38), 8.137 (4.19), 11.644 (1.04).

### Example 103

### (2E)-1-[2-(4-chloro-2-fluorophenyl)-3-(1H-pyrrolo[2,3-b]pyridin-4-yl)-6,7-dihydropyrazolo[1,5-a]pyrazin-5(4H)-yl]-4-(dimethylamino)but-2-en-1-one

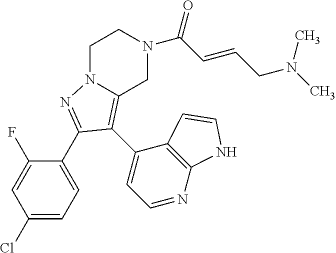 [SMILES: CN(C)C/C=C/C(=O)N1CCn2nc(-c3ccc(Cl)cc3F)c(-c3ccnc4[nH]ccc34)c2C1]

2-(4-chloro-2-fluorophenyl)-3-(1H-pyrrolo[2,3-b]pyridin-4-yl)-4,5,6,7-tetrahydropyrazolo[1,5-a]pyrazine-hydrogen chloride (1/2) (82.0 mg, 186 μmol, Intermediate 167) was dissolved in DMF (530 μl), then N,N-diisopropylethylamine (190 μl, 1.1 mmol; CAS-RN: [7087-68-5]), (2E)-4-(dimethylamino)but-2-enoic acid-hydrogen chloride (1/1) (33.9 mg, 205 μmol) and T3P (160 μl, 50% purity in DMF, 280 μmol, CAS-RN: [68957-94-8]) were added, and the mixture was stirred at RT for 30 minutes Water was added and the pH adjusted to 10 using sodium hydroxide solution, and the aqueous layer was extracted with ethyl acetate (2×). Purification by basic reverse phase prep HPLC C18, yielded the title compound (40.0 mg, 95% purity, 43% yield)

LC-MS (Method 2): Rₜ=1.00 min; MS (ESIpos): m/z=479 [M+H]⁺

¹H-NMR (400 MHz, DMSO-d6) δ [ppm]: 2.038 (3.55), 2.075 (1.53), 2.150 (16.00), 2.318 (1.27), 2.518 (14.26), 2.523 (10.27), 2.660 (1.21), 2.954 (0.95), 3.043 (3.08), 3.056 (2.86), 4.128 (0.94), 4.190 (2.74), 4.343 (2.91), 4.763 (4.28), 4.868 (1.08), 5.952 (3.41), 6.508 (0.42), 6.633 (0.90), 6.672 (1.23), 6.741 (1.71), 6.775 (2.79), 7.273 (3.46), 7.287 (6.07), 7.307 (3.62), 7.349 (3.34), 7.442 (1.74), 7.462 (2.79), 7.483 (1.38), 8.151 (11.75), 8.164 (10.78), 11.674 (2.85).

### Example 104

### 1-[2-(4-chloro-2-fluorophenyl)-3-(1H-pyrrolo[2,3-b]pyridin-4-yl)-6,7-dihydropyrazolo[1,5-a]pyrazin-5(4H)-yl]prop-2-en-1-one

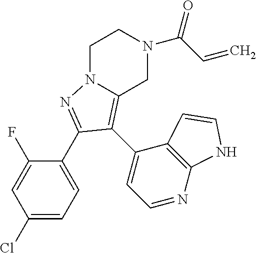 [SMILES: C=CC(=O)N1CCn2nc(-c3ccc(Cl)cc3F)c(-c3ccnc4[nH]ccc34)c2C1]

2-(4-chloro-2-fluorophenyl)-3-(1H-pyrrolo[2,3-b]pyridin-4-yl)-4,5,6,7-tetrahydropyrazolo[1,5-a]pyrazine-hydrogen chloride (1/2) (82.0 mg, 186 μmol, Intermediate 167) was dissolved in DMF (530 μl), then N,N-diisopropylethylamine (190 μl, 1.1 mmol; CAS-RN: [7087-68-5]), prop-2-enoic acid (14 μl, 200 μmol) and T3P (160 μl, 50% purity in DMF, 280 μmol, CAS-RN: [68957-94-8]) were added, and the mixture was stirred at RT for 30 minutes. Water was added and the pH adjusted to 10 using sodium hydroxide solution, and the aqueous layer was extracted with ethyl acetate (2×). Purification by basic reverse phase prep HPLC C18, yielded the title compound (27.0 mg, 95% purity, 33% yield)

LC-MS (Method 2): Rₜ=1.00 min; MS (ESIpos): m/z=422 [M+H]⁺

¹H-NMR (400 MHz, DMSO-d6) δ[ppm]: 1.237 (0.62), 1.352 (0.43), 2.074 (1.49), 2.318 (1.39), 2.518 (16.00), 2.523 (11.99), 2.660 (1.44), 3.301 (1.07), 3.314 (1.98), 3.365 (1.92), 3.377 (0.78), 4.132 (0.82), 4.209 (2.46), 4.350 (2.64), 4.779 (4.34), 4.885 (1.02), 5.765 (1.19), 5.793 (1.16), 5.959 (2.04), 6.143 (1.36), 6.187 (1.28), 6.768 (1.83), 6.779 (1.88), 6.843 (0.54), 6.922 (0.83), 6.948 (0.84), 6.963 (0.82), 6.990 (0.68), 7.274 (2.82), 7.289 (4.11), 7.309 (2.48), 7.353 (2.34), 7.445 (1.53), 7.467 (2.44), 7.487 (1.20), 8.155 (9.68), 8.167 (9.04), 11.680 (1.84).

The following examples were prepared analogously to Example 102 from the stated starting material and prop-2-enoic acid using T3P as the amide coupling reagent in DMF using DIPEA as the base.

| | Structure |
|---|---|
| | IUPAC-Name |
| | LC-MS (method): Retention time; Mass found |
| Example | ¹H-NMR |
| Example 105 | 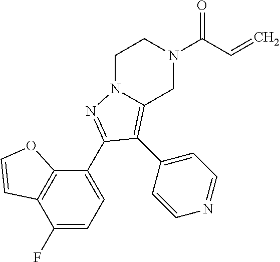 [SMILES: C=CC(=O)N1CCn2nc(-c3ccc(F)c4ccoc34)c(-c3ccncc3)c2C1] |
| | 1-[2-(4-fluoro-1-benzofuran-7-yl)-3-(pyridin-4-yl)-6,7- |
| | dihydropyrazolo[1,5-a]pyrazin-5(4H)-yl]prop-2-en-1-one |
| | Produced from 2-(4-fluoro-1-benzofuran-7-yl)-3-(pyridin-4-yl)-4,5,6,7- |
| | tetrahydropyrazolo[1,5-a]pyrazin-5-ium chloride (Intermediate 168) |
| | analogously. |
| | LC-MS (Method 24): Rₜ = 0.94 min; MS (ESlpos): m/z = 389 [M + H]⁺ |
| | 1H-NMR (400 MHz, DMSO-d6) delta [ppm]: 1.230 (0.64), 2.518 (2.00), |
| | 2.523 (1.32), 4.126 (1.36), 4.200 (2.82), 4.252 (0.68), 4.286 (1.45), 4.335 |
| | (3.09), 4.374 (0.64), 4.407 (0.41), 4.712 (0.64), 4.962 (4.55), 5.090 |
| | (1.45), 5.788 (1.59), 5.813 (1.36), 6.092 (0.41), 6.098 (0.45), 6.152 |
| | (0.45), 6.177 (2.55), 6.195 (0.59), 6.219 (2.59), 6.249 (0.59), 6.252 |
| | (0.45), 6.944 (1.36), 6.971 (1.36), 6.986 (1.23), 7.026 (5.00), 7.049 |
| | (2.86), 7.056 (16.00), 7.062 (14.91), 7.134 (4.18), 7.155 (6.41), 7.157 |
| | (5.50), 7.179 (5.32), 7.191 (0.50), 7.207 (0.59), 7.226 (0.91), 7.242 |
| | (1.00), 7.255 (1.00), 7.265 (1.05), 7.278 (1.14), 7.291 (2.77), 7.304 |
| | (2.86), 7.311 (2.36), 7.325 (1.77), 7.803 (0.64), 7.808 (0.86), 7.827 |
| | (8.32), 7.832 (8.59), 8.414 (4.55), 8.483 (0.41), 9.786 (1.41). |
| Example 106 | 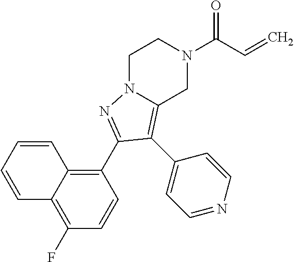 [SMILES: C=CC(=O)N1CCn2nc(-c3ccc(F)c4ccccc34)c(-c3ccncc3)c2C1] |
| | 1-[2-(4-fluoronaphthalen-1-yl)-3-(pyridin-4-yl)-6,7- |
| | dihydropyrazolo[1,5-a]pyrazin-5(4H)-yl]prop-2-en-1-one |
| | Produced from 2-(4-fluoro-1-naphthyl)-3-(pyridin-4-yl)-4,5,6,7- |
| | tetrahydropyrazolo[1,5-a]pyrazin-5-ium chloride (Intermediate 169) |
| | analogously. |
| | LC-MS (Method 24): Rₜ = 1.03 min; MS (ESlpos): m/z = 399 [M + H]⁺ |
| | 1H-NMR (400 MHz, METHANOL-d4) delta [ppm]: 2.660 (0.70), 4.281 |
| | (3.98), 4.294 (8.20), 4.308 (6.56), 4.421 (3.08), 4.624 (4.82), 5.127 |
| | (4.37), 5.174 (1.44), 5.868 (1.29), 5.894 (1.14), 6.317 (1.39), 6.359 |
| | (1.54), 6.922 (0.75), 6.947 (0.80), 6.962 (0.70), 6.989 (0.70), 7.041 |
| | (16.00), 7.045 (9.04), 7.052 (8.94), 7.057 (15.80), 7.230 (4.07), 7.250 |
| | (5.17), 7.256 (4.27), 7.276 (4.72), 7.402 (2.53), 7.405 (2.63), 7.419 |
| | (3.73), 7.423 (5.32), 7.427 (3.88), 7.432 (3.78), 7.441 (4.82), 7.445 |
| | (6.51), 7.452 (3.28), 7.465 (2.88), 7.533 (3.18), 7.535 (3.33), 7.551 |
| | (3.03), 7.554 (4.87), 7.556 (3.63), 7.571 (2.78), 7.574 (2.63), 7.749 |
| | (3.68), 7.751 (4.27), 7.773 (3.68), 8.115 (5.47), 8.136 (5.07), 8.261 |
| | (14.66), 8.265 (8.60), 8.272 (8.50), 8.276 (13.17). |
| Example 107 | 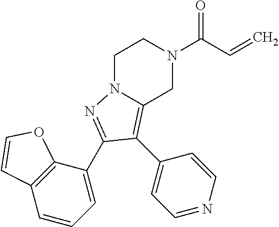 [SMILES: C=CC(=O)N1CCn2nc(-c3cccc4ccoc34)c(-c3ccncc3)c2C1] |
| | 1-[2-(1-benzofuran-7-yl)-3-(pyridin-4-yl)-6,7-dihydropyrazolo[1,5- |
| | alpyrazin-5(4H)-yl]prop-2-en-1-one |
| | Produced from 2-(1-benzofuran-7-yl)-3-(pyridin-4-yl)-4,5,6,7- |
| | tetrahydropyrazolo[1,5-a]pyrazin-5-ium chloride (Intermediate 170) |
| | analogously. |
| | LC-MS (Method 24): Rₜ = 0.90 min; MS (ESlpos): m/z = 371 [M + H]⁺ |
| | ¹H-NMR (400 MHz, METHANOL-d4) δ [ppm]: 0.088 (0.41), 1.398 (0.46), |
| | 2.660 (1.27), 4.250 (3.78), 4.263 (7.61), 4.277 (5.99), 4.400 (2.64), 4.626 |
| | (13.72), 4.917 (2.93), 4.944 (0.51), 5.062 (3.92), 5.112 (1.22), 5.855 |
| | (1.12), 5.880 (1.00), 6.301 (1.20), 6.342 (1.24), 6.818 (12.30), 6.823 |
| | (13.52), 6.903 (0.63), 6.931 (0.70), 6.970 (0.46), 7.127 (16.00), 7.131 |
| | (8.64), 7.138 (9.05), 7.143 (15.75), 7.278 (5.03), 7.297 (10.66), 7.316 |
| | (7.95), 7.367 (4.98), 7.370 (5.31), 7.386 (3.33), 7.388 (3.10), 7.480 |
| | (7.25), 7.485 (6.92), 7.662 (5.86), 7.665 (5.93), 7.681 (5.76), 7.684 |
| | (5.31), 8.335 (12.97), 8.339 (7.38), 8.347 (7.40), 8.351 (11.74). |
| Example 108 | 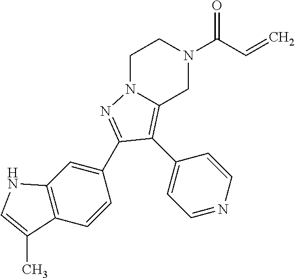 [SMILES: C=CC(=O)N1CCn2nc(-c3ccc4c(C)c[nH]c4c3)c(-c3ccncc3)c2C1] |
| | 1-[2-(3-methyl-1H-indol-6-yl)-3-(pyridin-4-yl)-6,7- |
| | dihydropyrazolo[1,5-a]pyrazin-5(4H)-yl]prop-2-en-1-one |
| | Produced from 2-(3-methyl-3H-indol-6-yl)-3-(pyridin-4-yl)-4,5,6,7- |
| | tetrahydropyrazolo[1,5-a]pyrazin-5-ium chloride (Intermediate 171) |
| | analogously. |
| | LC-MS (Method 24): Rₜ = 0.93 min; MS (ESlpos): m/z = 384 [M + H]⁺ |
| | ¹H-NMR (400 MHz, METHANOL-d4) δ [ppm]: 0.098 (0.45), 2.295 |
| | (16.00), 2.298 (15.74), 2.656 (0.74), 3.330 (0.56), 3.334 (0.44), 4.214 |
| | (1.45), 4.228 (3.27), 4.242 (2.70), 4.340 (1.22), 4.622 (0.48), 4.979 |
| | (1.80), 5.023 (0.57), 5.841 (0.50), 5.866 (0.45), 6.288 (0.56), 6.329 |
| | (0.55), 7.021 (5.72), 7.024 (5.63), 7.042 (1.02), 7.240 (6.36), 7.244 |
| | (3.92), 7.251 (3.81), 7.255 (6.86), 7.339 (3.67), 7.341 (3.63), 7.343 |
| | (3.13), 7.449 (2.60), 7.470 (2.35), 8.417 (6.84), 8.421 (3.53), 8.428 |
| | (3.71), 8.433 (6.07). |
| Example 109 | 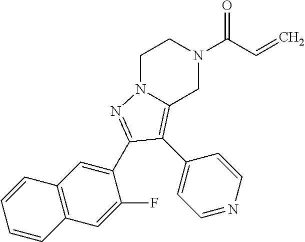 [SMILES: C=CC(=O)N1CCn2nc(-c3cc4ccccc4cc3F)c(-c3ccncc3)c2C1] |
| | 1-[2-(3-fluoronaphthalen-2-yl)-3-(pyridin-4-yl)-6,7- |
| | dihydropyrazolo[1,5-a]pyrazin-5(4H)-yl]prop-2-en-1-one |
| | Produced from 2-(3-fluoro-2-naphthyl)-3-(pyridin-4-yl)-4,5,6,7- |
| | tetrahydropyrazolo[1,5-a]pyrazin-5-ium chloride (Intermediate 172) |
| | analogously. |
| | LC-MS (Method 24): Rₜ = 1.01 min; MS (ESlpos): m/z = 399 [M + H]⁺ |
| | 1H-NMR (400 MHz, METHANOL-d4) delta [ppm]: 0.098 (0.92), 1.399 |
| | (0.81), 2.658 (1.73), 3.330 (1.45), 3.334 (1.17), 4.252 (3.51), 4.266 |
| | (7.14), 4.280 (5.46), 4.395 (2.48), 4.406 (2.51), 4.908 (0.92), 5.066 |
| | (3.62), 5.119 (1.14), 5.856 (1.06), 5.882 (0.95), 6.304 (1.17), 6.345 |
| | (1.20), 6.934 (0.64), 7.187 (15.97), 7.192 (8.72), 7.199 (9.03), 7.203 |
| | (16.00), 7.480 (1.62), 7.483 (1.70), 7.497 (3.29), 7.500 (4.15), 7.520 |
| | (7.83), 7.539 (3.15), 7.547 (5.02), 7.558 (3.87), 7.575 (1.78), 7.847 |
| | (4.32), 7.867 (3.87), 7.899 (4.29), 7.917 (3.82), 7.919 (3.79), 8.051 |
| | (5.55), 8.069 (5.46), 8.400 (13.80), 8.405 (7.89), 8.412 (7.53), 8.416 |
| | (13.44). |
| Example 110 | 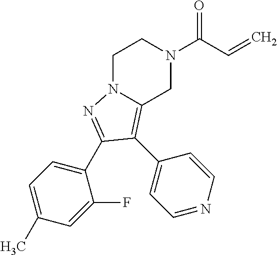 [SMILES: C=CC(=O)N1CCn2nc(-c3ccc(C)cc3F)c(-c3ccncc3)c2C1] |
| | 1-[2-(2-fluoro-4-methylphenyl)-3-(pyridin-4-yl)-6,7- |
| | dihydropyrazolo[1,5-a]pyrazin-5(4H)-yl]prop-2-en-1-one |
| | Produced from 2-(2-fluoro-4-methylphenyl)-3-(pyridin-4-yl)-4,5,6,7- |
| | tetrahydropyrazolo[1,5-a]pyrazin-5-ium chloride (Intermediate 173) |
| | analogously. |
| | LC-MS (Method 24): Rₜ = 0.94 min; MS (ESlpos): m/z = 363 [M + H]⁺ |
| | 1H-NMR (400 MHz, METHANOL-d4) delta [ppm]: 0.098 (0.80), 1.241 |
| | (0.80), 1.289 (0.80), 1.400 (1.01), 2.014 (1.33), 2.354 (3.33), 2.363 |
| | (2.35), 2.387 (16.00), 3.330 (1.57), 3.333 (1.12), 3.808 (0.64), 4.224 |
| | (1.71), 4.236 (3.17), 4.251 (2.53), 4.348 (1.31), 4.618 (1.63), 5.024 |
| | (1.52), 5.639 (2.48), 6.159 (1.52), 6.172 (0.67), 6.176 (0.77), 6.208 |
| | (2.45), 6.220 (1.07), 6.225 (1.09), 6.288 (0.72), 6.329 (0.77), 6.869 |
| | (0.72), 6.891 (0.83), 6.916 (1.84), 6.944 (1.57), 7.072 (1.92), 7.091 |
| | (2.13), 7.160 (1.55), 7.164 (0.91), 7.175 (7.15), 7.179 (3.65), 7.186 |
| | (3.39), 7.191 (6.05), 7.310 (0.93), 7.319 (0.88), 7.326 (1.07), 7.332 |
| | (0.99), 7.338 (2.03), 7.351 (1.71), 7.357 (3.09), 7.370 (0.88), 7.376 |
| | (1.49), 7.404 (2.61), 7.407 (1.60), 7.415 (1.63), 7.419 (2.53), 8.411 |
| | (1.92), 8.415 (4.08), 8.420 (6.61), 8.424 (3.87), 8.426 (3.63), 8.431 |
| | (5.55), 8.436 (4.93), 8.495 (0.85), 8.510 (0.75), 9.794 (1.65). |
| Example 111 | 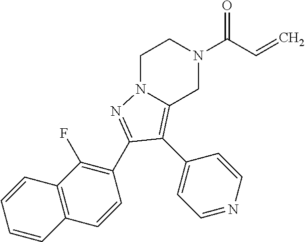 [SMILES: C=CC(=O)N1CCn2nc(-c3ccc4ccccc4c3F)c(-c3ccncc3)c2C1] |
| | 1-[2-(1-fluoronaphthalen-2-yl)-3-(pyridin-4-yl)-6,7- |
| | dihydropyrazolo[1,5-a]pyrazin-5(4H)-yl]prop-2-en-1-one |
| | Produced from 2-(1-fluoro-2-naphthyl)-3-(pyridin-4-yl)-4,5,6,7- |
| | tetrahydropyrazolo[1,5-a]pyrazin-5-ium chloride (Intermediate 174) |
| | analogously. |
| | LC-MS (Method 24): Rₜ = 1.03 min; MS (ESlpos): m/z = 399 [M + H]⁺ |
| | 1H-NMR (400 MHz, METHANOL-d4) delta [ppm]: 0.089 (0.73), 0.098 |
| | (1.19), 1.287 (0.81), 1.400 (1.33), 2.013 (0.76), 2.659 (2.54), 3.133 |
| | (0.65), 3.330 (2.00), 3.333 (1.46), 4.254 (3.95), 4.267 (7.82), 4.281 |
| | (6.12), 4.407 (2.82), 4.617 (0.79), 5.067 (3.84), 5.118 (1.25), 5.689 |
| | (1.54), 5.853 (1.22), 5.880 (1.06), 6.178 (0.95), 6.225 (1.62), 6.237 |
| | (0.79), 6.243 (0.76), 6.303 (1.35), 6.344 (1.38), 6.907 (0.73), 6.933 |
| | (0.79), 7.194 (1.49), 7.201 (16.00), 7.205 (9.31), 7.212 (9.20), 7.217 |
| | (15.95), 7.431 (0.84), 7.447 (0.87), 7.528 (4.30), 7.546 (5.93), 7.550 |
| | (6.71), 7.567 (8.23), 7.582 (6.69), 7.587 (6.90), 7.602 (3.76), 7.605 |
| | (4.95), 7.619 (2.06), 7.623 (1.76), 7.758 (7.42), 7.779 (5.96), 7.942 |
| | (7.72), 7.963 (7.36), 8.400 (15.21), 8.404 (8.37), 8.411 (9.53), 8.415 |
| | (13.56), 9.841 (1.03). |
| Example 112 | 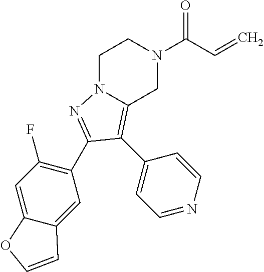 [SMILES: C=CC(=O)N1CCn2nc(-c3cc4ccoc4cc3F)c(-c3ccncc3)c2C1] |
| | 1-[2-(6-fluoro-1-benzofuran-5-yl)-3-(pyridin-4-yl)-6,7- |
| | dihydropyrazolo[1,5-a]pyrazin-5(4H)-yl]prop-2-en-1-one |
| | Produced from 2-(6-fluoro-1-benzofuran-5-yl)-3-(pyridin-4-yl)-4,5,6,7- |
| | tetrahydropyrazolo[1,5-a]pyrazin-5-ium chloride (Intermediate 175) |
| | analogously. |
| | LC-MS (Method 24): Rₜ = 0.92 min; MS (ESlpos): m/z = 389 [M + H]⁺ |
| | ¹H-NMR (400 MHz, METHANOL-d4) δ [ppm]: 0.088 (0.52), 0.098 (1.10), |
| | 1.287 (0.60), 1.399 (1.10), 2.013 (0.45), 2.660 (1.57), 3.133 (0.47), 3.330 |
| | (1.69), 3.334 (1.34), 3.482 (0.49), 4.243 (3.71), 4.256 (7.63), 4.270 |
| | (6.00), 4.382 (2.86), 4.621 (0.79), 5.052 (4.06), 5.102 (1.31), 5.849 |
| | (1.21), 5.876 (1.06), 6.297 (1.31), 6.339 (1.34), 6.897 (9.13), 6.899 |
| | (9.85), 6.903 (9.50), 6.905 (9.17), 6.925 (0.79), 6.965 (0.53), 7.169 |
| | (16.00), 7.173 (8.69), 7.180 (9.38), 7.185 (15.17), 7.305 (4.86), 7.330 |
| | (4.89), 7.714 (8.54), 7.732 (8.21), 7.823 (12.73), 7.829 (12.38), 8.401 |
| | (13.31), 8.406 (7.61), 8.413 (7.76), 8.417 (12.30). |
| Example 113 | 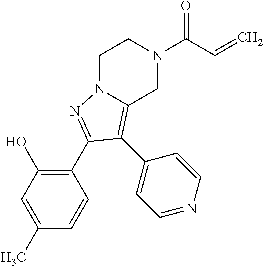 [SMILES: C=CC(=O)N1CCn2nc(-c3ccc(C)cc3O)c(-c3ccncc3)c2C1] |
| | 1-[2-(2-hydroxy-4-methylphenyl)-3-(pyridin-4-yl)-6,7- |
| | dihydropyrazolo[1,5-a]pyrazin-5(4H)-yl]prop-2-en-1-one |
| | Produced from 2-(2-hydroxy-4-methylphenyl)-3-(pyridin-4-yl)-4,5,6,7- |
| | tetrahydropyrazolo[1,5-a]pyrazin-5-ium chloride (Intermediate 176) |
| | analagously. |
| | LC-MS (Method 24): Rₜ = 0.93 min; MS (ESlpos): m/z = 361 [M + H]⁺ |
| | ¹H-NMR (400 MHz, METHANOL-d4) δ [ppm]: 0.098 (0.50), 1.223 (0.91), |
| | 1.240 (1.94), 1.258 (0.94), 1.400 (0.50), 2.014 (3.22), 2.285 (16.00), |
| | 2.402 (0.40), 3.330 (0.63), 3.334 (0.50), 3.788 (0.66), 4.091 (0.65), 4.109 |
| | (0.64), 4.209 (1.59), 4.222 (3.35), 4.236 (2.73), 4.327 (1.32), 4.912 |
| | (0.44), 4.974 (1.87), 5.021 (0.60), 5.835 (0.55), 5.860 (0.54), 6.282 |
| | (0.59), 6.322 (0.59), 6.625 (1.57), 6.627 (1.65), 6.644 (1.69), 6.646 |
| | (1.80), 6.687 (3.32), 7.027 (1.84), 7.046 (1.63), 7.253 (6.41), 7.257 |
| | (3.48), 7.264 (3.80), 7.269 (6.21), 8.434 (5.63), 8.439 (3.38), 8.446 |
| | (3.33), 8.450 (5.39). |
| Example 114 | 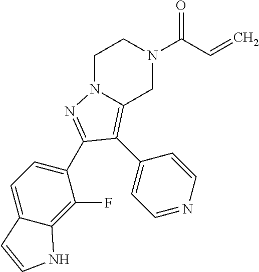 [SMILES: C=CC(=O)N1CCn2nc(-c3ccc4cc[nH]c4c3F)c(-c3ccncc3)c2C1] |
| | 1-[2-(7-fluoro-1H-indol-6-yl)-3-(pyridin-4-yl)-6,7-dihydropyrazolo[1,5- |
| | alpyrazin-5(4H)-yl]prop-2-en-1-one |
| | Produced from 2-(7-fluoro-1H-indol-6-yl)-3-(pyridin-4-yl)-4,5,6,7- |
| | tetrahydropyrazolo[1,5-a]pyrazin-5-ium chloride (Intermediate 177) |
| | analogously. |
| | LC-MS (Method 24): Rₜ = 0.87 min; MS (ESlpos): m/z = 388 [M + H]⁺ |
| | ¹H-NMR (400 MHz, METHANOL-d4) δ [ppm]: 0.089 (0.67), 0.098 (1.06), |
| | 1.222 (3.38), 1.240 (6.65), 1.258 (3.07), 1.288 (0.99), 1.400 (1.24), 2.013 |
| | (11.11), 3.129 (0.40), 3.133 (0.55), 3.137 (0.41), 3.330 (1.75), 3.334 |
| | (1.42), 3.482 (0.58), 3.788 (2.57), 4.073 (0.84), 4.091 (2.47), 4.109 |
| | (2.46), 4.126 (0.83), 4.240 (3.88), 4.253 (8.10), 4.268 (6.51), 4.375 |
| | (3.08), 4.621 (1.02), 5.055 (4.34), 5.104 (1.36), 5.847 (1.30), 5.874 |
| | (1.16), 6.297 (1.39), 6.339 (1.42), 6.527 (5.71), 6.535 (9.24), 6.544 |
| | (5.73), 6.898 (0.76), 6.925 (0.86), 6.965 (0.54), 7.031 (3.12), 7.046 |
| | (3.65), 7.051 (3.74), 7.066 (3.42), 7.189 (15.74), 7.193 (9.24), 7.200 |
| | (8.84), 7.205 (16.00), 7.299 (13.84), 7.307 (13.36), 7.399 (12.18), 7.419 |
| | (10.68), 8.380 (14.08), 8.384 (8.56), 8.392 (8.42), 8.395 (13.75). |
| Example 115 | 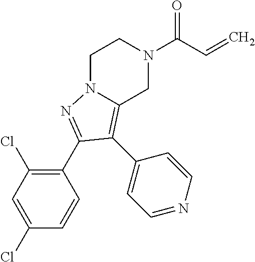 [SMILES: C=CC(=O)N1CCn2nc(-c3ccc(Cl)cc3Cl)c(-c3ccncc3)c2C1] |
| | 1-[2-(2,4-dichlorophenyl)-3-(pyridin-4-yl)-6,7-dihydropyrazolo[1,5- |
| | a]pyrazin-5(4H)-yl]prop-2-en-1-one |
| | Produced from 2-(2,4-dichlorophenyl)-3-(pyridin-4-yl)-4,5,6,7- |
| | tetrahydropyrazolo[1,5-a]pyrazin-5-ium chloride (Intermediate 178) |
| | analogously. |
| | LC-MS (Method 24): Rₜ = 1.03 min; MS (ESlpos): m/z = 399 [M + H]⁺ |
| | ¹H-NMR (400 MHz, METHANOL-d4) δ [ppm]: 0.089 (0.60), 0.099 (1.01), |
| | 1.288 (0.65), 1.362 (0.50), 1.379 (0.48), 1.400 (1.10), 2.660 (2.91), 3.133 |
| | (0.55), 3.330 (1.84), 3.333 (1.31), 3.482 (0.50), 4.239 (3.60), 4.252 |
| | (7.39), 4.266 (6.06), 4.369 (3.01), 4.617 (1.80), 5.066 (4.04), 5.113 |
| | (1.33), 5.846 (1.23), 5.873 (1.09), 6.293 (1.33), 6.335 (1.40), 6.893 |
| | (0.70), 6.921 (0.80), 6.962 (0.48), 7.126 (15.24), 7.130 (9.28), 7.138 |
| | (9.43), 7.141 (16.00), 7.432 (2.59), 7.437 (2.99), 7.453 (10.60), 7.457 |
| | (10.94), 7.468 (13.00), 7.488 (3.17), 7.525 (9.39), 7.529 (8.38), 8.420 |
| | (14.25), 8.425 (8.67), 8.433 (8.41), 8.436 (14.05). |
| Example 116 | 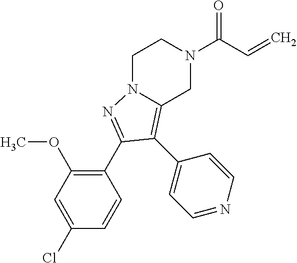 [SMILES: C=CC(=O)N1CCn2nc(-c3ccc(Cl)cc3OC)c(-c3ccncc3)c2C1] |
| | 1-[2-(4-chloro-2-methoxyphenyl)-3-(pyridin-4-yl)-6,7- |
| | dihydropyrazolo[1,5-a]pyrazin-5(4H)-yl]prop-2-en-1-one |
| | Produced from 2-(4-chloro-2-methoxyphenyl)-3-(pyridin-4-yl)-4,5,6,7- |
| | tetrahydropyrazolo[1,5-a]pyrazin-5-ium chloride (Intermediate 179) |
| | analogously. |
| | LC-MS (Method 24): Rₜ = 0.97 min; MS (ESlpos): m/z = 395 [M + H]⁺ |
| | ¹H-NMR (400 MHz, METHANOL-d4) δ [ppm]: 1.400 (0.44), 3.330 (1.24), |
| | 3.334 (1.28), 3.343 (16.00), 4.217 (1.25), 4.229 (2.77), 4.244 (2.32), |
| | 4.336 (1.12), 4.617 (0.70), 4.870 (0.89), 5.023 (1.57), 5.071 (0.52), 5.840 |
| | (0.48), 5.867 (0.42), 6.289 (0.49), 6.328 (0.51), 6.982 (3.08), 6.986 |
| | (3.29), 7.053 (2.31), 7.057 (2.04), 7.073 (2.50), 7.077 (2.31), 7.144 |
| | (5.45), 7.148 (3.18), 7.155 (3.32), 7.159 (5.40), 7.389 (3.54), 7.409 |
| | (3.13), 8.420 (5.25), 8.424 (2.97), 8.431 (3.01), 8.436 (4.98). |
| Example 117 | 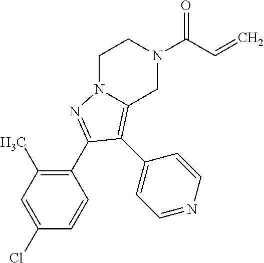 [SMILES: C=CC(=O)N1CCn2nc(-c3ccc(Cl)cc3C)c(-c3ccncc3)c2C1] |
| | 1-[2-(4-chloro-2-methylphenyl)-3-(pyridin-4-yl)-6,7- |
| | dihydropyrazolo[1,5-a]pyrazin-5(4H)-yl]prop-2-en-1-one |
| | Produced from 2-(4-chloro-2-methylphenyl)-3-(pyridin-4-yl)-4,5,6,7- |
| | tetrahydropyrazolo[1,5-a]pyrazin-5-ium chloride (Intermediate 180) |
| | analogously |
| | LC-MS (Method 24): Rₜ = 1.03 min; MS (ESlpos): m/z = 379 [M + H]⁺ |
| | ¹H-NMR (400 MHz, METHANOL-d4) δ [ppm]: 0.099 (0.70), 1.289 (0.41), |
| | 1.400 (0.73), 2.020 (0.78), 2.035 (16.00), 2.094 (0.54), 2.660 (0.57), |
| | 3.284 (0.69), 3.330 (0.64), 3.335 (0.50), 3.340 (0.96), 3.343 (0.76), 3.348 |
| | (0.41), 4.237 (1.44), 4.250 (3.13), 4.264 (2.63), 4.356 (1.30), 4.617 |
| | (1.46), 4.850 (0.51), 4.865 (1.54), 4.875 (5.34), 4.911 (0.45), 4.921 |
| | (3.19), 5.074 (1.78), 5.117 (0.67), 5.849 (0.54), 5.875 (0.47), 6.296 |
| | (0.57), 6.338 (0.63), 7.109 (6.13), 7.114 (3.68), 7.121 (3.71), 7.125 |
| | (6.29), 7.212 (0.99), 7.232 (4.07), 7.242 (2.92), 7.247 (2.78), 7.262 |
| | (0.60), 7.267 (0.81), 7.295 (3.30), 8.404 (5.84), 8.408 (3.52), 8.416 |
| | (3.45), 8.420 (5.69). |
| Example 118 | 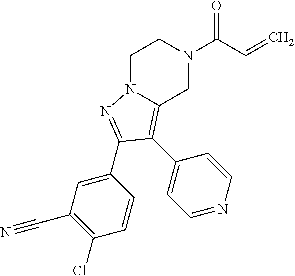 [SMILES: C=CC(=O)N1CCn2nc(-c3ccc(Cl)c(C#N)c3)c(-c3ccncc3)c2C1] |
| | 2-chloro-5-[5-(prop-2-enoyl)-3-(pyridin-4-yl)-4,5,6,7- |
| | tetrahydropyrazolo[1,5-a]pyrazin-2-yl]benzonitrile |
| | Produced from 2-(4-chloro-3-cyanophenyl)-3-(pyridin-4-yl)-4,5,6,7- |
| | tetrahydropyrazolo[1,5-a]pyrazin-5-ium chloride (Intermediate 181) |
| | analogously. |
| | LC-MS (Method 24): Rₜ = 0.98 min; MS (ESlpos): m/z = 390 [M + H]⁺ |
| | ¹H-NMR (400 MHz, DMSO-d6) δ [ppm]: 0.958 (1.66), 0.974 (1.55), 1.231 |
| | (0.94), 2.518 (3.27), 2.523 (2.21), 4.091 (1.83), 4.165 (4.29), 4.265 |
| | (2.09), 4.310 (4.62), 4.687 (0.46), 4.847 (7.13), 4.981 (2.21), 5.778 |
| | (2.23), 5.803 (2.02), 6.160 (3.41), 6.202 (3.81), 6.927 (1.60), 6.953 |
| | (1.46), 6.969 (1.30), 6.995 (1.03), 7.218 (8.94), 7.369 (0.90), 7.373 |
| | (0.64), 7.380 (0.73), 7.384 (1.07), 7.539 (0.40), 7.544 (0.41), 7.597 |
| | (3.53), 7.619 (4.48), 7.730 (16.00), 7.751 (11.65), 7.893 (5.48), 8.542 |
| | (0.40), 8.583 (10.12), 8.594 (8.45), 8.644 (0.74), 8.647 (0.49), 8.654 |
| | (0.41), 8.659 (0.74), 9.676 (1.34). |
| Example 119 | 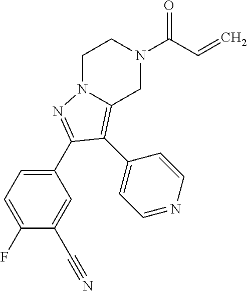 [SMILES: C=CC(=O)N1CCn2nc(-c3ccc(F)c(C#N)c3)c(-c3ccncc3)c2C1] |
| | 2-fluoro-5-[5-(prop-2-enoyl)-3-(pyridin-4-yl)-4,5,6,7- |
| | tetrahydropyrazolo[1,5-a]pyrazin-2-yl]benzonitrile |
| | Produced from 2-(3-cyano-4-fluorophenyl)-3-(pyridin-4-yl)-4,5,6,7- |
| | tetrahydropyrazolo[1,5-a]pyrazin-5-ium chloride (Intermediate 182) |
| | analogously. |
| | LC-MS (Method 24): Rₜ = 0.91 min; MS (ESlpos): m/z = 374 [M + H]⁺ |
| | ¹H-NMR (400 MHz, METHANOL-d4) δ [ppm]: 0.088 (0.49), 0.091 (0.43), |
| | 0.099 (1.10), 1.288 (0.52), 1.399 (1.17), 3.133 (0.44), 3.329 (1.74), 3.334 |
| | (1.32), 3.482 (0.46), 4.213 (3.49), 4.226 (6.99), 4.240 (5.52), 4.362 |
| | (2.92), 4.622 (4.67), 4.947 (3.88), 5.000 (1.20), 5.841 (1.18), 5.867 |
| | (1.09), 6.283 (1.24), 6.324 (1.36), 6.882 (0.62), 6.908 (0.67), 6.923 |
| | (0.65), 6.950 (0.46), 7.276 (15.30), 7.280 (9.20), 7.288 (8.74), 7.291 |
| | (16.00), 7.326 (4.18), 7.348 (8.59), 7.371 (4.63), 7.658 (2.13), 7.664 |
| | (2.35), 7.671 (2.23), 7.677 (2.54), 7.681 (2.28), 7.686 (2.16), 7.693 |
| | (1.88), 7.699 (1.97), 7.801 (2.24), 7.813 (2.13), 8.529 (13.60), 8.534 |
| | (8.05), 8.541 (7.94), 8.545 (12.63). |
| Example 120 | 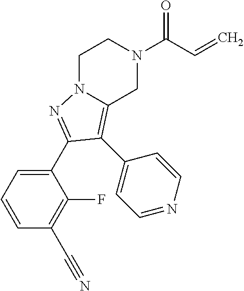 [SMILES: C=CC(=O)N1CCn2nc(-c3cccc(C#N)c3F)c(-c3ccncc3)c2C1] |
| | 2-fluoro-3-[5-(prop-2-enoyl)-3-(pyridin-4-yl)-4,5,6,7- |
| | tetrahydropyrazolo[1,5-a]pyrazin-2-yl]benzonitrile |
| | Produced from 2-(3-cyano-2-fluorophenyl)-3-(pyridin-4-yl)-4,5,6,7- |
| | tetrahydropyrazolo[1,5-a]pyrazin-5-ium chloride (Intermediate 183) |
| | analogously. |
| | LC-MS (Method 24): Rₜ = 0.86 min; MS (ESlpos): m/z = 374 [M + H]⁺ |
| | ¹H-NMR (400 MHz, METHANOL-d4) delta [ppm]: 0.088 (0.75), 0.091 |
| | (0.64), 0.099 (1.81), 1.288 (0.75), 1.358 (1.71), 1.375 (1.71), 1.400 |
| | (1.81), 1.918 (0.53), 3.130 (0.53), 3.133 (0.85), 3.137 (0.53), 3.330 |
| | (2.99), 3.334 (2.13), 3.478 (0.64), 3.482 (0.85), 3.487 (0.64), 4.235 |
| | (3.84), 4.248 (7.47), 4.262 (5.76), 4.393 (2.99), 4.452 (0.53), 4.619 |
| | (9.17), 4.847 (0.85), 4.930 (0.64), 5.030 (3.84), 5.084 (1.28), 5.645 |
| | (0.85), 5.845 (1.28), 5.873 (1.07), 6.208 (0.96), 6.293 (1.28), 6.333 |
| | (1.39), 6.891 (0.75), 7.184 (0.85), 7.197 (15.79), 7.201 (9.49), 7.208 |
| | (8.96), 7.212 (16.00), 7.426 (4.80), 7.445 (9.81), 7.464 (5.44), 7.802 |
| | (2.24), 7.806 (5.01), 7.813 (5.12), 7.817 (4.69), 7.822 (6.51), 7.826 |
| | (4.69), 7.830 (5.65), 7.836 (4.48), 7.841 (3.73), 7.849 (3.84), 7.854 |
| | (2.03), 8.483 (13.87), 8.487 (8.21), 8.495 (8.00), 8.499 (12.16), 8.542 |
| | (0.64). |
| Example 121 | 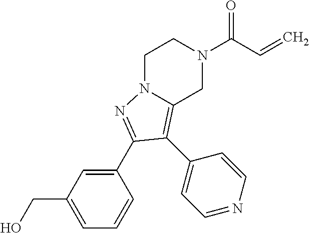 [SMILES: C=CC(=O)N1CCn2nc(-c3cccc(CO)c3)c(-c3ccncc3)c2C1] |
| | 1-{2-[3-(hydroxymethyl)phenyl]-3-(pyridin-4-yl)-6,7- |
| | dihydropyrazolo[1,5-a]pyrazin-5(4H)-yl}prop-2-en-1-one |
| | Produced from 2-[3-(hydroxymethyl)phenyl]-3-(pyridin-4-yl)-4,5,6,7- |
| | tetrahydropyrazolo[1,5-a]pyrazin-5-ium chloride (Intermediate 184) |
| | analogously. |
| | LC-MS (Method 3): Rₜ = 0.58 min; MS (ESlpos): m/z = 361 [M + H]⁺ |
| | ¹H-NMR (400 MHz, METHANOL-d4) δ [ppm]: 1.145 (3.15), 1.161 (5.37), |
| | 1.179 (5.73), 1.196 (3.10), 1.288 (0.72), 2.659 (0.42), 3.580 (0.61), 3.598 |
| | (1.75), 3.615 (1.76), 3.633 (0.60), 4.213 (2.66), 4.225 (5.70), 4.239 |
| | (4.62), 4.344 (2.12), 4.561 (16.00), 4.965 (3.12), 5.017 (0.98), 5.838 |
| | (0.93), 5.865 (0.84), 6.284 (1.00), 6.325 (1.03), 6.884 (0.53), 6.911 |
| | (0.55), 6.925 (0.50), 7.244 (12.14), 7.248 (7.19), 7.256 (8.50), 7.259 |
| | (13.59), 7.298 (2.22), 7.317 (4.97), 7.336 (3.21), 7.348 (4.37), 7.367 |
| | (1.65), 7.437 (4.21), 7.458 (0.40), 7.463 (0.59), 8.457 (8.03), 8.461 |
| | (5.13), 8.468 (5.08), 8.472 (7.65). NMR contains traces of ethanol. |
| Example 122 | 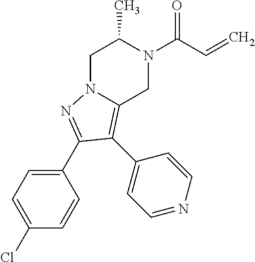 [SMILES: C=CC(=O)N1Cc2c(-c3ccncc3)c(-c3ccc(Cl)cc3)nn2C[C@@H]1C] |
| | 1-[(6S)-2-(4-chlorophenyl)-6-methyl-3-(pyridin-4-yl)-6,7- |
| | dihydropyrazolo[1,5-a]pyrazin-5(4H)-yl]prop-2-en-1-one |
| | Produced from (6S)-2-(4-chlorophenyl)-6-methyl-3-(pyridin-4-yl)-4,5,6,7- |
| | tetrahydropyrazolo[1,5-a]pyrazine hydrogen chloride (1/1) (Intermediate |
| | 432) analogously. |
| | LC-MS (Method 24): Rₜ = 1.02 min; MS (ESlpos): m/z = 379 [M + H]⁺ |
| | ¹H-NMR (400 MHz, METHANOL-d4) δ [ppm]: 0.098 (0.50), 1.251 (0.43), |
| | 1.255 (0.42), 1.267 (0.46), 1.272 (0.48), 1.286 (1.31), 1.304 (1.11), 1.327 |
| | (3.24), 1.342 (3.27), 1.399 (0.57), 3.329 (0.86), 3.334 (0.68), 4.269 |
| | (1.51), 4.301 (2.66), 4.386 (0.57), 4.624 (1.26), 4.935 (0.50), 5.817 |
| | (1.23), 5.844 (1.28), 6.269 (2.06), 6.273 (2.10), 6.311 (2.40), 6.315 |
| | (2.42), 7.272 (0.51), 7.278 (1.08), 7.290 (5.58), 7.293 (3.93), 7.301 |
| | (3.81), 7.305 (5.77), 7.313 (2.28), 7.348 (2.27), 7.350 (1.88), 7.355 |
| | (1.55), 7.364 (2.20), 7.371 (16.00), 7.380 (14.76), 7.386 (1.91), 7.395 |
| | (1.23), 7.400 (1.73), 7.402 (2.17), 8.499 (3.01), 8.511 (2.89), 9.684 |
| | (0.54). |
| Example 123 | 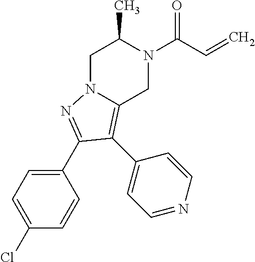 [SMILES: C=CC(=O)N1Cc2c(-c3ccncc3)c(-c3ccc(Cl)cc3)nn2C[C@H]1C] |
| | 1-[(6R)-2-(4-chlorophenyl)-6-methyl-3-(pyridin-4-yl)-6,7- |
| | dihydropyrazolo[1,5-a]pyrazin-5(4H)-yl]prop-2-en-1-one |
| | Produced from (6R)-2-(4-chlorophenyl)-6-methyl-3-(pyridin-4-yl)-4,5,6,7- |
| | tetrahydropyrazolo[1,5-a]pyrazine hydrogen chloride (1/1) (Intermediate |
| | Intermediate 436) anagously. |
| | LC-MS (Method 2): Rₜ = 1.10 min; MS (ESlpos): m/z = 379 [M + H]⁺ |
| | ¹H-NMR (400 MHz, METHANOL-d4) δ [ppm]: 1.161 (1.09), 1.179 (1.89), |
| | 1.196 (1.02), 1.287 (1.81), 1.326 (3.04), 1.342 (2.99), 1.937 (0.57), 2.271 |
| | (0.68), 2.660 (7.93), 3.597 (0.57), 3.615 (0.60), 4.268 (1.36), 4.299 |
| | (2.52), 4.386 (0.50), 4.621 (0.50), 4.912 (1.00), 4.923 (0.44), 5.815 |
| | (1.14), 5.843 (1.19), 6.269 (1.98), 6.273 (1.99), 6.311 (2.32), 6.315 |
| | (2.30), 7.290 (6.44), 7.293 (4.00), 7.301 (3.99), 7.305 (6.52), 7.313 |
| | (0.51), 7.349 (2.13), 7.351 (1.70), 7.355 (1.19), 7.365 (1.98), 7.371 |
| | (16.00), 7.380 (13.73), 7.386 (1.63), 7.395 (1.08), 7.400 (1.62), 7.402 |
| | (2.01), 8.496 (3.54), 8.511 (3.37). |
| Example 124 | 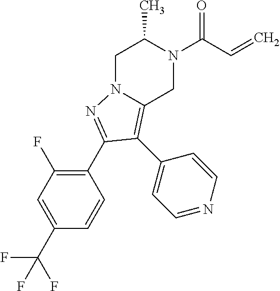 [SMILES: C=CC(=O)N1Cc2c(-c3ccncc3)c(-c3ccc(C(F)(F)F)cc3F)nn2C[C@@H]1C] |
| | 1-[(6S)-2-[2-fluoro-4-(trifluoromethyl)phenyl]-6-methyl-3-(pyridin-4- |
| | yl)-6,7-dihydropyrazolo[1,5-a]pyrazin-5(4H)-yl]prop-2-en-1-one |
| | Porduced from (6S)-2-[2-fluoro-4-(trifluoromethyl)phenyl]-6-methyl-3- |
| | (pyridin-4-yl)-4,5,6,7-tetrahydropyrazolo[1,5-a]pyrazin-5-ium chloride |
| | (Intermediate 186) analogously. |
| | LC-MS (Method 2): Rₜ = 1.13 min; MS (ESlpos): m/z = 431 [M + H]⁺ |
| | ¹H-NMR (400 MHz, METHANOL-d4) δ [ppm]: 1.337 (1.40), 1.353 (1.42); |
| | 2.660 (16.00), 4.317 (0.64), 4.349 (1.13), 4.375 (0.50), 5.824 (0.60), |
| | 5.850 (0.76), 6.279 (1.02), 6.283 (1.03), 6.321 (1.18), 6.325 (1.19), 7.226 |
| | (3.56), 7.230 (2.24), 7.238 (2.10), 7.242 (3.81), 7.456 (0.91), 7.458 |
| | (0.95), 7.480 (0.90), 7.483 (0.93), 7.592 (1.01), 7.613 (1.10), 7.615 |
| | (1.07), 7.742 (0.73), 7.762 (1.14), 7.780 (0.58), 8.463 (3.20), 8.467 |
| | (1.98), 8.475 (1.89), 8.478 (3.16). |
| Example 125 | 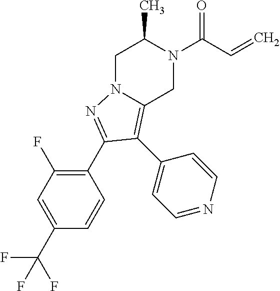 [SMILES: C=CC(=O)N1Cc2c(-c3ccncc3)c(-c3ccc(C(F)(F)F)cc3F)nn2C[C@H]1C] |
| | 1-[(6R)-2-[2-fluoro-4-(trifluoromethyl)phenyl]-6-methyl-3-(pyridin-4- |
| | yl)-6,7-dihydropyrazolo[1,5-a]pyrazin-5(4H)-yl]prop-2-en-1-one |
| | Produced from (6R)-2-[2-fluoro-4-(trifluoromethyl)phenyl]-6-methyl-3- |
| | (pyridin-4-yl)-4,5,6,7-tetrahydropyrazolo[1,5-a]pyrazin-5-ium chloride |
| | (Intermediate 185) analogously. |
| | LC-MS (Method 2): Rₜ = 1.13 min; MS (ESlpos): m/z = 431 [M + H]⁺ |
| Example 126 | 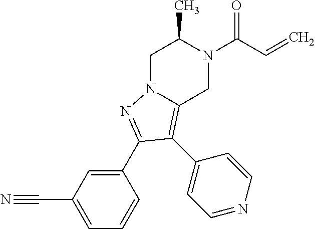 [SMILES: C=CC(=O)N1Cc2c(-c3ccncc3)c(-c3cccc(C#N)c3)nn2C[C@H]1C] |
| | 3-[(6R)-6-methyl-5-(prop-2-enoyl)-3-(pyridin-4-yl)-4,5,6,7- |
| | tetrahydropyrazolo[1,5-a]pyrazin-2-yl]benzonitrile |
| | Produced from 3-[(6R)-6-methyl-3-(pyridin-4-yl)-4,5,6,7- |
| | tetrahydropyrazolo[1,5-a]pyrazin-2-yl]benzonitrile hydrochloride (1:1) |
| | (Intermediate 187) analogously |
| | LC-MS (Method 2): Rₜ = 0.92 min; MS (ESlpos): m/z = 370 [M + H]⁺ |
| | 1H-NMR (400 MHz, DMSO-d6) delta [ppm]: 1.234 (4.45), 1.352 (0.66), |
| | 2.518 (16.00), 2.523 (10.36), 4.248 (2.26), 4.279 (4.32), 4.517 (0.53), |
| | 4.636 (0.66), 4.935 (0.60), 5.146 (5.38), 5.189 (4.45), 5.753 (2.26), 5.780 |
| | (2.66), 5.811 (0.40), 6.155 (4.78), 6.161 (4.71), 6.197 (5.18), 6.202 |
| | (5.31), 7.221 (10.62), 7.236 (10.69), 7.358 (4.58), 7.389 (11.22), 7.391 |
| | (11.42), 7.396 (6.04), 7.403 (8.30), 7.422 (1.26), 7.617 (0.73), 7.637 |
| | (0.60), 7.693 (0.53), 7.811 (3.39), 7.815 (3.78), 7.819 (3.52), 7.825 |
| | (5.38), 7.833 (3.25), 7.837 (3.45), 7.900 (0.93), 7.919 (0.53), 7.995 |
| | (11.42), 8.528 (9.03), 8.543 (8.70), 8.967 (0.60), 8.983 (0.53). |
| Example 127 | 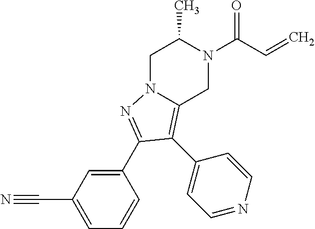 [SMILES: C=CC(=O)N1Cc2c(-c3ccncc3)c(-c3cccc(C#N)c3)nn2C[C@@H]1C] |
| | 3-[(6S)-6-methyl-5-(prop-2-enoyl)-3-(pyridin-4-yl)-4,5,6,7- |
| | tetrahydropyrazolo[1,5-a]pyrazin-2-yl]benzonitrile |
| | Produced from 3-[(6S)-6-methyl-3-(pyridin-4-yl)-4,5,6,7- |
| | tetrahydropyrazolo[1,5-a]pyrazin-2-yl]benzonitrile hydrochloride (1:1) |
| | (Intermediate 188) analogously |
| | LC-MS (Method 3): Rₜ = 0.71 min; MS (ESlpos): m/z = 370[M + H]⁺ |
| | 1H-NMR (400 MHz, DMSO-d6) delta [ppm]: 1.153 (1.14), 1.169 (1.52), |
| | 1.231 (5.86), 1.351 (0.54), 2.083 (0.60), 2.518 (12.69), 2.523 (8.35), |
| | 2.659 (1.03), 3.586 (0.43), 4.253 (3.15), 4.284 (6.07), 4.350 (1.25), 4.503 |
| | (0.76), 4.522 (0.71), 4.944 (0.76), 5.133 (7.59), 5.177 (6.13), 5.214 |
| | (0.49), 5.758 (5.80), 5.779 (3.20), 6.151 (7.11), 6.157 (7.11), 6.193 |
| | (7.97), 6.199 (8.08), 6.919 (1.19), 7.253 (15.73), 7.257 (10.90), 7.264 |
| | (10.74), 7.268 (16.00), 7.287 (0.49), 7.291 (0.60), 7.320 (0.60), 7.335 |
| | (0.60), 7.529 (0.60), 7.538 (0.54), 7.547 (4.94), 7.567 (11.88), 7.586 |
| | (8.62), 7.629 (5.80), 7.633 (9.17), 7.636 (6.67), 7.649 (3.74), 7.653 |
| | (5.53), 7.657 (3.69), 7.769 (10.41), 7.803 (6.73), 7.806 (9.38), 7.810 |
| | (5.75), 7.822 (5.75), 7.826 (7.86), 7.829 (4.88), 8.427 (0.65), 8.533 |
| | (0.65), 8.548 (0.71), 8.568 (13.56), 8.582 (13.18), 8.622 (0.54), 8.637 |
| | (0.54), 9.706 (0.81). |

### Example 128

### 1-{2-[4-(difluoromethoxy)phenyl]-3-(pyridin-4-yl)-6,7-dihydropyrazolo[1,5-a]pyrazin-5(4H)-yl}prop-2-en-1-one

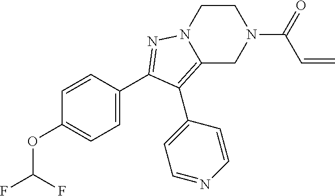 [SMILES: C=CC(=O)N1CCn2nc(-c3ccc(OC(F)F)cc3)c(-c3ccncc3)c2C1]

To a solution of 2-(4-(difluoromethoxy)phenyl)-3-(pyridin-4-yl)-4,5,6,7-tetrahydropyrazolo[1,5-a]pyrazine hydrochloride (1:1) (150 mg, 0.395 mmol, Intermediate 191) and acryloyl chloride (43.0 mg, 0.475 mmol) in dichloromethane (3.6 ml) was added triethylamine (80.1 mg, 0.761 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 27%-57%, 9 min) to give 1-(2-{[4-(difluoromethoxy)phenyl]-3-(pyridin-4-yl)-6,7-dihydropyrazolo[1,5-a]pyrazin-5(4H)-yl}prop-2-en-1-one (5.00 mg, 0.0122 mmol, 3% yield) as a white solid.

LC-MS (Method 20): Rₜ=0.878 min; MS (ESIpos): m/z=396.1 [M+H]⁺.

¹H NMR (400 MHz, DMSO-d6) δ [ppm]=8.55 (d, J=3.6 Hz, 2H), 7.44 (s, 1H), 7.39 (d, J=8.4 Hz, 2H), 7.26 (s, 1H), 7.21-7.12 (m, 4H), 7.08 (s, 1H), 7.02-6.83 (m, 1H), 6.17 (d, J=16.8 Hz, 1H), 5.84-5.68 (m, 1H), 5.03-4.74 (m, 2H), 4.36-4.19 (m, 2H), 4.18-4.05 (m, 2H).

### Example 129

### 1-[3-(pyridin-4-yl)-2-[4-(trifluoromethoxy)phenyl]-6,7-dihydropyrazolo[1,5-a]pyrazin-5(4H)-yl]prop-2-en-1-one

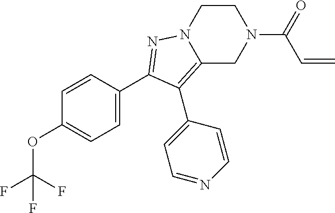 [SMILES: C=CC(=O)N1CCn2nc(-c3ccc(OC(F)(F)F)cc3)c(-c3ccncc3)c2C1]

To a solution of 3-(pyridin-4-yl)-2-[4-(trifluoromethoxy)phenyl]-4,5,6,7-tetrahydropyrazolo[1,5-a]pyrazine hydrochloride (1:1) (110 mg, 0.277 mmol, Intermediate 193) and acryloyl chloride (33.1 mg, 0.336 mmol) in dichloromethane (2.2 ml) was added triethylamine (61.7 mg, 0.610 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 35%-65%, 9 min) to give 1-[3-(pyridin-4-yl)-2-[4-(trifluoromethoxy)phenyl]-6,7-dihydropyrazolo[1,5-a]pyrazin-5(4H)-yl]prop-2-en-1-one (39.7 mg, 0.0937 mmol, 30% yield) as a gray solid.

LC-MS (Method 20): Rₜ=0.605 min; MS (ESIpos): m/z=414.1 [M+H]⁺.

¹H NMR (400 MHz, DMSO-d6) δ [ppm]=8.56 (d, J=4.0 Hz, 2H), 7.46 (d, J=8.4 Hz, 2H), 7.35 (d, J=8.4 Hz, 2H), 7.19 (d, J=3.6 Hz, 2H), 7.00-6.82 (m, 1H), 6.17 (d, J=16.4 Hz, 1H), 5.83-5.69 (m, 1H), 4.99-4.80 (m, 2H), 4.29 (br s, 2H), 4.19-4.04 (m, 2H).

### Example 130

### 1-[2-(4-ethoxyphenyl)-3-(pyridin-4-yl)-6,7-dihydropyrazolo[1,5-a]pyrazin-5(4H)-yl]prop-2-en-1-one

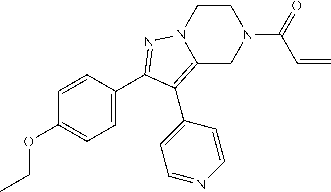 [SMILES: C=CC(=O)N1CCn2nc(-c3ccc(OCC)cc3)c(-c3ccncc3)c2C1]

To a solution of 2-(4-ethoxyphenyl)-3-(pyridin-4-yl)-4,5,6,7-tetrahydropyrazolo[1,5-a]pyrazine hydrochloride (1:1) (150 mg, 0.420 mmol, Intermediate 195) and acryloyl chloride (45.6 mg, 0.504 mmol) in dichloromethane (2.6 ml) was added triethylamine (85.0 mg, 0.840 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 28%-58%, 9 min) to give 1-[2-(4-ethoxyphenyl)-3-(pyridin-4-yl)-6,7-dihydropyrazolo[1,5-a]pyrazin-5(4H)-yl]prop-2-en-1-one (43.7 mg, 0.0115 mmol, 27% yield) as a white solid.

LC-MS (Method 20): Rₜ=0.525 min; MS (ESIpos): m/z=374.1 [M+H]⁺.

¹H NMR (400 MHz, DMSO-d6) δ [ppm]=8.53 (br s, 2H), 7.26 (d, J=8.4 Hz, 2H), 7.17 (br s, 2H), 7.01-6.84 (m, 3H), 6.18 (d, J=16.4 Hz, 1H), 5.84-5.68 (m, 1H), 5.00-4.73 (m, 2H), 4.31-4.20 (m, 2H), 4.19-4.07 (m, 2H), 4.02 (q, J=8.0 Hz, 2H), 1.36-1.28 (m, 3H).

### Example 131

### 1-{2-[4-(difluoromethyl)phenyl]-3-(pyridin-4-yl)-6,7-dihydropyrazolo[1,5-a]pyrazin-5(4H)-yl}prop-2-en-1-one

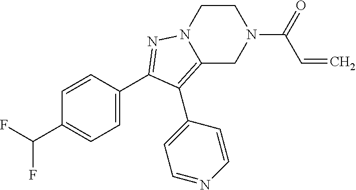 [SMILES: C=CC(=O)N1CCn2nc(-c3ccc(C(F)F)cc3)c(-c3ccncc3)c2C1]

To a solution of 2-[4-(difluoromethyl)phenyl]-3-(pyridin-4-yl)-4,5,6,7-tetrahydropyrazolo[1,5-a]pyrazine hydrochloride (1:1) (110 mg, 0.303 mmol, Intermediate 197) and prop-2-enoyl chloride (32.9 mg, 0.364 mmol) in dichloromethane (2 ml) were added trimethylamine (61.3 mg, 0.606 mmol) at 20° C. The mixture was stirred at 20° C. for 16 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 22%-52%, 9 min) to give 1-{2-[4-(difluoromethyl)phenyl]-3-(pyridin-4-yl)-6,7-dihydropyrazolo[1,5-a]pyrazin-5(4H)-yl}prop-2-en-1-one (12.7 mg, 0.0331 mmol, 10% yield) as a gray solid.

LC-MS (Method 21): Rₜ=0.915 min; MS (ESIpos): m/z=380.1 [M+H]⁺.

¹H NMR (400 MHz, DMSO-d6) δ [ppm]=8.56 (d, J=4.8 Hz, 2H), 7.60-7.53 (m, 3H), 7.52-7.46 (m, 2H), 7.18 (d, J=5.6 Hz, 2H), 7.18-7.17 (m, 1H), 7.04 (s, 1H), 7.01-6.91 (m, 1H), 6.90 (s, 1H), 6.18 (d, J=16.0 Hz, 1H), 5.85-5.69 (m, 1H), 5.02-4.79 (m, 2H), 4.37-4.23 (m, 2H), 4.22-4.06 (m, 2H).

### Example 132

### 1-[2-(5-chloro-2-fluorophenyl)-3-(pyridin-4-yl)-6,7-dihydropyrazolo[1,5-a]pyrazin-5(4H)-yl]prop-2-en-1-one

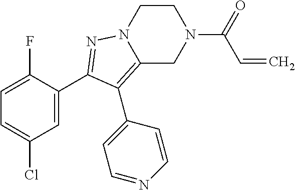 [SMILES: C=CC(=O)N1CCn2nc(-c3cc(Cl)ccc3F)c(-c3ccncc3)c2C1]

To a solution of 2-(5-chloro-2-fluorophenyl)-3-(pyridin-4-yl)-4,5,6,7-tetrahydropyrazolo[1,5-a]pyrazine hydrochloride (1:1) (150 mg, 0.411 mmol, Intermediate 199) and prop-2-enoyl chloride (44.6 mg, 0.493 mmol) in dichloromethane (3.6 ml) was added trimethylamine (83.1 mg, 0.821 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 27%-57%, 9 min) to give 1-[2-(5-chloro-2-fluorophenyl)-3-(pyridin-4-yl)-6,7-dihydropyrazolo[1,5-a]pyrazin-5(4H)-yl]prop-2-en-1-one (33.5 mg, 0.0869 mmol, 99% purity, 21% yield) as an orange solid.

LC-MS (Method 21): Rₜ=0.854 min; MS (ESIpos): m/z=382.1 [M+H]⁺.

¹H NMR (400 MHz, DMSO-d6) δ [ppm]=8.51 (d, J=4.4 Hz, 2H), 7.57-7.50 (m, 2H), 7.31-7.23 (m, 1H), 7.10 (br s, 2H), 7.03-6.87 (m, 1H), 6.19 (d, J=18.0 Hz, 1H), 5.88-5.68 (m, 1H), 5.11-4.87 (m, 2H), 4.32 (br s, 2H), 4.22-4.07 (m, 2H).

### Example 133

### 1-[2-(3-chlorophenyl)-3-(pyridin-4-yl)-6,7-dihydropyrazolo[1,5-a]pyrazin-5(4H)-yl]prop-2-en-1-one

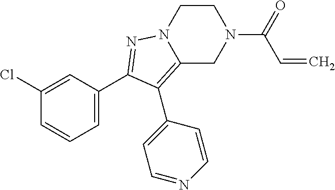 [SMILES: C=CC(=O)N1CCn2nc(-c3cccc(Cl)c3)c(-c3ccncc3)c2C1]

To a solution of 2-(3-chlorophenyl)-3-(pyridin-4-yl)-4,5,6,7-tetrahydropyrazolo[1,5-a]pyrazine hydrochloride (1:1) (100 mg, 0.288 mmol, Intermediate 201) and prop-2-enoyl chloride (31.3 mg, 0.346 mmol) in dichloromethane (2.0 ml) was added trimethylamine (58.2 mg, 0.575 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 26%-56%, 9 min) to give 1-[2-(3-chlorophenyl)-3-(pyridin-4-yl)-6,7-dihydropyrazolo[1,5-a]pyrazin-5(4H)-yl]prop-2-en-1-one (14.5 mg, 0.0368 mmol, 13% yield) as a white solid.

LC-MS (Method 21): Rₜ=0.922 min; MS (ESIpos): m/z=364.1 [M+H]⁺.

¹H NMR (400 MHz, DMSO-d6) δ [ppm]=8.70-8.63 (m, 2H), 7.47 (dd, J=4.8, 1.6 Hz, 2H), 7.43-7.33 (m, 3H), 7.24 (d, J=7.2 Hz, 1H), 6.98-6.79 (m, 1H), 6.15 (dd, J=16.4, 2.0 Hz, 1H), 5.75 (d, J=9.6 Hz, 1H), 5.07-4.82 (m, 2H), 4.29 (d, J=7.2 Hz, 2H), 4.16-4.04 (m, 2H).

### Example 134

### 1-{2-[3-fluoro-4-(trifluoromethyl)phenyl]-3-(pyridin-4-yl)-6,7-dihydropyrazolo[1,5-a]pyrazin-5(4H)-yl}prop-2-en-1-one

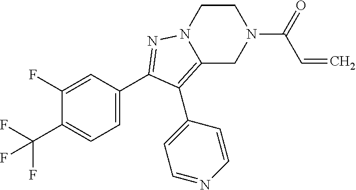 [SMILES: C=CC(=O)N1CCn2nc(-c3ccc(C(F)(F)F)c(F)c3)c(-c3ccncc3)c2C1]

To a solution of 2-[3-fluoro-4-(trifluoromethyl)phenyl]-3-(pyridin-4-yl)-4,5,6,7-tetrahydropyrazolo[1,5-a]pyrazine hydrochloride (1:1) (100 mg, 0.251 mmol, Intermediate 203) and prop-2-enoyl chloride (27.2 mg, 0.301 mmol) in dichloromethane (2.7 ml) was added triethylamine (50.7 mg, 0.501 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 33%-63%, 9 min) to give 1-{2-[3-fluoro-4-(trifluoromethyl)phenyl]-3-(pyridin-4-yl)-6,7-dihydropyrazolo[1,5-a]pyrazin-5(4H)-yl}prop-2-en-1-one (21.0 mg, 0.0499 mmol, 19% yield) as a yellow solid.

LC-MS (Method 21): Rₜ=0.989 min; MS (ESIpos): m/z=316.1 [M+H]⁺.

¹H NMR (400 MHz, DMSO-da) δ [ppm]=8.60 (d, J=3.6 Hz, 2H), 7.77 (t, J=7.6 Hz, 1H), 7.43 (d, J=12.4 Hz, 1H), 7.33 (d, J=7.6 Hz, 1H), 7.25 (br s, 2H), 7.02-6.82 (m, 1H), 6.18 (d, J=16.4 Hz, 1H), 5.79 (d, J=10.4 Hz, 1H), 5.01-4.78 (m, 2H), 4.40-4.24 (m, 2H), 4.22-4.06 (m, 2H).

### Example 135

### 1-{2-[2-fluoro-4-(trifluoromethyl)phenyl]-3-(pyridin-4-yl)-6,7-dihydropyrazolo[1,5-a]pyrazin-5(4H)-yl}prop-2-en-1-one

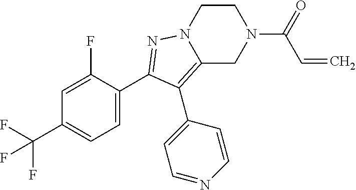 [SMILES: C=CC(=O)N1CCn2nc(-c3ccc(C(F)(F)F)cc3F)c(-c3ccncc3)c2C1]

To a solution of 2-[2-fluoro-4-(trifluoromethyl)phenyl]-3-(pyridin-4-yl)-4,5,6,7-tetrahydropyrazolo[1,5-a]pyrazine hydrochloride (1:1) (150 mg, 0.376 mmol, Intermediate 205) and prop-2-enoyl chloride (40.9 mg, 0.451 mmol) in dichloromethane (2.7 ml) was added triethylamine (76.1 mg, 0.750 mmol) at 20° C. The mixture was stirred at 20° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 32%-62%, 9 min) to give 1-{2-[2-fluoro-4-(trifluoromethyl)phenyl]-3-(pyridin-4-yl)-6,7-dihydropyrazolo[1,5-a]pyrazin-5(4H)-yl}prop-2-en-1-one (22.0 mg, 0.0516 mmol, 13% yield) as a gray solid.

LC-MS (Method 21): Rₜ=0.962 min; MS (ESIpos): m/z=316.1 [M+H]⁺.

¹H NMR (400 MHz, DMSO-d6) δ [ppm]=8.51 (d, J=4.0 Hz, 2H), 7.78-7.65 (m, 3H), 7.10 (br s, 2H), 6.96 (dd, J=15.6, 9.2 Hz, 1H), 6.18 (d, J=16.8 Hz, 1H), 5.83-5.70 (m, 1H), 5.09-4.89 (m, 2H), 4.36-4.25 (m, 2H), 4.23-4.07 (m, 2H).

### Example 136

### 1-[2-(2-fluorophenyl)-3-(pyridin-4-yl)-6,7-dihydropyrazolo[1,5-a]pyrazin-5(4H)-yl]prop-2-en-1-one

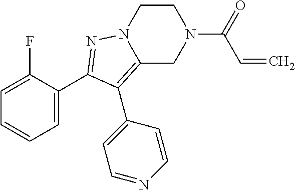 [SMILES: C=CC(=O)N1CCn2nc(-c3ccccc3F)c(-c3ccncc3)c2C1]

To a solution of 2-(2-fluorophenyl)-3-(pyridin-4-yl)-4,5,6,7-tetrahydropyrazolo[1,5-a]pyrazine hydrogen chloride (1:1) (100 mg, 302 μmol, Intermediate 207) and trimethylamine (130 μl, 910 μmol) in dichloromethane (5 ml) was added acryloyl chloride (27.4 mg, 302 μmol) at 20° C. The mixture was stirred at 20° C. for 1 hour. The reaction mixture was concentrated under vacuum to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 22-52% B; flow 25 ml/min; temperature: RT; Detector: UV 220/254 nm] to give 1-[2-(2-fluorophenyl)-3-(pyridin-4-yl)-6,7-dihydropyrazolo[1,5-a]pyrazin-5(4H)-yl]prop-2-en-1-one (29.9 mg, 84.1 μmol, 98% purity, 28% yield) as a grey solid.

LC-MS (Method 20): Rₜ=0.825 min; MS (ESIpos): m/z=349.0 [M+H]⁺

¹H NMR (400 MHz, DMSO-d6) δ [ppm]=8.48 (s, 2H), 7.57-7.40 (m, 2H), 7.32-7.25 (m, 1H), 7.23-7.16 (m, 1H), 7.06 (s, 2H), 7.01-6.90 (m, 1H), 6.19 (d, J=16.4 Hz, 1H), 5.89-5.58 (m, 1H), 5.20-4.82 (m, 2H), 4.43-4.00 (m, 4H).

### Example 137

### 1-[2-(2-chlorophenyl)-3-(pyridin-4-yl)-6,7-dihydropyrazolo[1,5-a]pyrazin-5(4H)-yl]prop-2-en-1-one

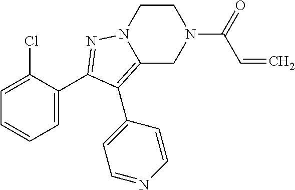 [SMILES: C=CC(=O)N1CCn2nc(-c3ccccc3Cl)c(-c3ccncc3)c2C1]

To a solution of 2-(2-chlorophenyl)-3-(pyridin-4-yl)-4,5,6,7-tetrahydropyrazolo[1,5-a]pyrazine hydrogen chloride (1:1) (100 mg, 288 μmol, Intermediate 209) in dichloromethane (5 ml) were added trimethylamine (120 μl, 860 μmol) and acryloyl chloride (26.1 mg, 288 μmol) at 25° C. The mixture was stirred at 25° C. for 1 hour. The reaction mixture was concentrated under vacuum to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 24-54% B; flow 25 ml/min; temperature: RT; Detector: UV 220/254 nm] to give 1-[2-(2-chlorophenyl)-3-(pyridin-4-yl)-6,7-dihydropyrazolo[1,5-a]pyrazin-5(4H)-yl]prop-2-en-1-one (14.9 mg, 40.4 μmol, 99% purity, 14% yield) as a white solid.

LC-MS (Method 20): Rₜ=0.857 min; MS (ESIpos): m/z=365.0 [M+H]⁺.

¹H NMR (400 MHz, DMSO-d6) δ [ppm]=8.44 (s, 2H), 7.58-7.32 (m, 4H), 7.07-6.85 (m, 3H), 6.29-6.07 (m, 1H), 5.85-5.64 (m, 1H), 5.17-4.84 (m, 2H), 4.39-4.05 (m, 4H).

### Example 138

### 1-[2-(4-methylphenyl)-3-(pyridin-4-yl)-6,7-dihydropyrazolo[1,5-a]pyrazin-5(4H)-yl]prop-2-en-1-one

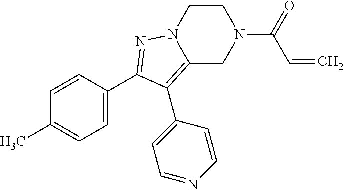 [SMILES: C=CC(=O)N1CCn2nc(-c3ccc(C)cc3)c(-c3ccncc3)c2C1]

To a solution of 2-(4-methylphenyl)-3-(pyridin-4-yl)-4,5,6,7-tetrahydropyrazolo[1,5-a]pyrazine hydrogen chloride (1:1) (100 mg, 306 μmol, Intermediate 213) in dichloromethane (5 ml) were added N,N-diisopropylethylamine (160 μl, 920 μmol) and acryloyl chloride (27.7 mg, 306 μmol) at 25° C. The mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 27-57% B; flow 25 ml/min; temperature: RT; Detector: UV 220/254 nm] to give 1-[2-(4-methylphenyl)-3-(pyridin-4-yl)-6,7-dihydropyrazolo[1,5-a]pyrazin-5(4H)-yl]prop-2-en-1-one (40.7 mg, 116 μmol, 98% purity, 38% yield) as a white solid.

LC-MS (Method 20): Rₜ=0.873 min; MS (ESIpos): m/z=345.1 [M+H]⁺.

¹H NMR (400 MHz, DMSO-d6) δ [ppm]=8.53 (s, 2H), 7.28-7.08 (m, 6H), 7.02-6.77 (m, 1H), 6.17 (d, J=16.4 Hz, 1H), 5.78 (d, J=9.6 Hz, 1H), 5.06-4.73 (m, 2H), 4.36-3.95 (m, 4H), 2.30 (s, 3H).

### Example 139

### 1-[2-(3-methylphenyl)-3-(pyridin-4-yl)-6,7-dihydropyrazolo[1,5-a]pyrazin-5(4H)-yl]prop-2-en-1-one

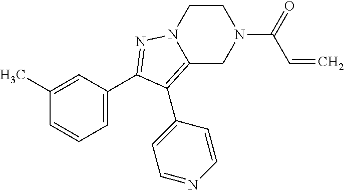 [SMILES: C=CC(=O)N1CCn2nc(-c3cccc(C)c3)c(-c3ccncc3)c2C1]

To a solution of 2-(3-methylphenyl)-3-(pyridin-4-yl)-4,5,6,7-tetrahydropyrazolo[1,5-a]pyrazine hydrogen chloride (1:1) (110 mg, 337 μmol, Intermediate 215) in dichloromethane (6 ml) were added N,N-diisopropylethylamine (180 μl, 1.0 mmol) and acryloyl chloride (30.5 mg, 337 μmol) at 25° C. The mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 27-57% B; flow 25 ml/min; temperature: RT; Detector: UV 220/254 nm] to give 1-[2-(3-methylphenyl)-3-(pyridin-4-yl)-6,7-dihydropyrazolo[1,5-a]pyrazin-5(4H)-yl]prop-2-en-1-one (38.2 mg, 110 μmol, 99% purity, 33% yield) as a grey solid.

LC-MS (Method 20): Rₜ=0.875 min; MS (ESIpos): m/z=345.1 [M+H]⁺

¹H NMR (400 MHz, DMSO-d6) δ[ppm]=8.53 (s, 2H), 7.29-7.11 (m, 5H), 7.05 (d, J=6.8 Hz, 1H), 7.00-6.80 (m, 1H), 6.18 (d, J=16.4 Hz, 1H), 5.78 (d, J=10.0 Hz, 1H), 5.02-4.76 (m, 2H), 4.33-4.01 (m, 4H), 2.26 (s, 3H).

### Example 140

### 1-[2-(4-ethylphenyl)-3-(pyridin-4-yl)-6,7-dihydropyrazolo[1,5-a]pyrazin-5(4H)-yl]prop-2-en-1-one

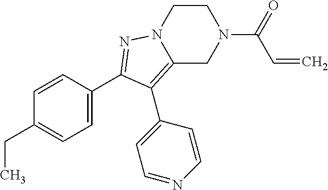 [SMILES: C=CC(=O)N1CCn2nc(-c3ccc(CC)cc3)c(-c3ccncc3)c2C1]

To a solution of 2-(4-ethylphenyl)-3-(pyridin-4-yl)-4,5,6,7-tetrahydropyrazolo[1,5-a]pyrazine hydrochloride (1:1) (200 mg, 587 mmol, Intermediate 217) in dichloromethane (5.0 ml) were added trimethylamine (178 mg, 1.76 mmol) and acryloyl chloride (53.1 mg, 587 mmol) at 25° C. The mixture was stirred at 25° C. for 1 hour. The reaction mixture was concentrated in vacuo to give a crude product. The crude product was purified by preparative HPLC ([Instrument: GX-A; Column: Waters Xbridge 150*25 mm*5 μm; eluent A: water (0.2% NH₃′H2O), eluent B: acetonitrile; gradient: 0-15 min 10-35% B; flow 25 ml/min; temperature: RT; Detector: UV 220/254 nm]) to give 1-[2-(4-ethylphenyl)-3-(pyridin-4-yl)-6,7-dihydropyrazolo[1,5-a]pyrazin-5(4H)-yl]prop-2-en-1-one (116 mg, 320 μmol, 99% purity, 55% yield) as a white solid.

LC-MS (Method 16): Rₜ=0.766 min; MS (ESIpos): m/z=359.2 [M+H]⁺

¹H NMR (400 MHz, DMSO-d6) δ [ppm]=8.54 (s, 2H), 7.26 (s, 2H), 7.19 (m, 4H), 7.04-6.83 (m, 1H), 6.18 (d, J=16.4 Hz, 1H), 5.79 (d, J=8.4 Hz, 1H), 5.01-4.78 (m, 2H), 4.27 (s, 2H), 4.19-4.01 (m, 2H), 2.66-2.56 (m, 2H), 1.24-1.15 (m, 3H).

### Example 141

### 3-[5-(prop-2-enoyl)-3-(pyridin-4-yl)-4,5,6,7-tetrahydropyrazolo[1,5-a]pyrazin-2-yl]benzonitrile

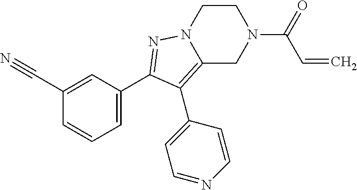 [SMILES: C=CC(=O)N1CCn2nc(-c3cccc(C#N)c3)c(-c3ccncc3)c2C1]

To a solution of 3-[3-(pyridin-4-yl)-4,5,6,7-tetrahydropyrazolo[1,5-a]pyrazin-2-yl]benzonitrile hydrogen chloride (1:1) (60.0 mg, 178 μmol, Intermediate 219) in dichloromethane (5 ml) were added N,N-diisopropylethylamine (93 μl, 530 μmol) and acryloyl chloride (16.1 mg, 178 μmol) at 25° C. The mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 26-56% B; flow 25 ml/min; temperature: RT; Detector: UV 220/254 nm] to give 3-[5-(prop-2-enoyl)-3-(pyridin-4-yl)-4,5,6,7-tetrahydropyrazolo[1,5-a]pyrazin-2-yl]benzonitrile (18.2 mg, 50.7 μmol, 99% purity, 29% yield) as a grey solid.

LC-MS (Method 20): Rₜ=0.829 min; MS (ESIpos): m/z=356.1 [M+H]⁺.

¹H NMR (400 MHz, DMSO-d6) δ [ppm]=8.58 (d, J=4.4 Hz, 2H), 7.85-7.78 (m, 1H), 7.76 (s, 1H), 7.67-7.60 (m, 1H), 7.60-7.52 (m, 1H), 7.19 (d, J=4.0 Hz, 2H), 7.04-6.77 (m, 1H), 6.18 (d, J=16.8 Hz, 1H), 5.85-5.61 (m, 1H), 5.04-4.74 (m, 2H), 4.40-4.00 (m, 4H).

### Example 142

### 1-[2-(2-fluoro-4-methoxyphenyl)-3-(pyridin-4-yl)-6,7-dihydropyrazolo[1,5-a]pyrazin-5(4H)-yl]prop-2-en-1-one

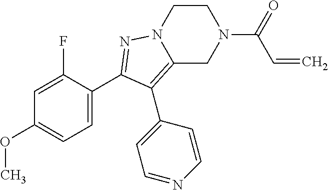 [SMILES: C=CC(=O)N1CCn2nc(-c3ccc(OC)cc3F)c(-c3ccncc3)c2C1]

To a solution of 2-(2-fluoro-4-methoxyphenyl)-3-(pyridin-4-yl)-4,5,6,7-tetrahydropyrazolo[1,5-a]pyrazine hydrogen chloride (1:1) (120 mg, 333 μmol, Intermediate 221) in dichloromethane (5 ml) were added N,N-diisopropylethylamine (170 μl, 1000 μmol) and acryloyl chloride (30.1 mg, 333 μmol) at 25° C. The mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 26-56% B; flow 25 ml/min; temperature: RT; Detector: UV 220/254 nm] to give 1-[2-(2-fluoro-4-methoxyphenyl)-3-(pyridin-4-yl)-6,7-dihydropyrazolo[1,5-a]pyrazin-5(4H)-yl]prop-2-en-1-one (47.9 mg, 124 μmol, 98% purity, 37% yield) as a grey solid.

LC-MS (Method 20): Rₜ=0.859 min; MS (ESIpos): m/z=379.1 [M+H]⁺.

¹H NMR (400 MHz, DMSO-d6) δ [ppm]=8.49 (d, J=4.4 Hz, 2H), 7.37 (t, J=8.8 Hz, 1H), 7.07 (s, 2H), 7.01-6.90 (m, 1H), 6.88-6.78 (m, 2H), 6.18 (d, J=16.8 Hz, 1H), 5.87-5.63 (m, 1H), 5.15-4.79 (m, 2H), 4.38-3.98 (m, 4H), 3.79 (s, 3H).

### Example 143

### 1-[3-(pyridin-4-yl)-2-{4-[(trifluoromethyl) sulfanyl]phenyl}-6,7-dihydropyrazolo[1,5-a]pyrazin-5(4H)-yl]prop-2-en-1-one

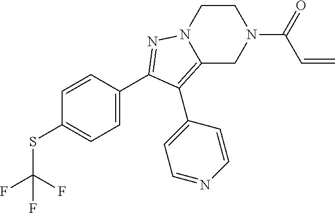 [SMILES: C=CC(=O)N1CCn2nc(-c3ccc(SC(F)(F)F)cc3)c(-c3ccncc3)c2C1]

To a solution of 3-(pyridin-4-yl)-2-(4-((trifluoromethyl)thio)phenyl)-4,5,6,7-tetrahydropyrazolo[1,5-a]pyrazine hydrochloride (1:1) (150 mg, 86% purity, 0.290 mmol Intermediate 223) and acryloyl chloride (30.0 mg, 0.320 mmol) in dichloromethane (2 ml) was added N,N-diisopropylethylamine (0.2 ml) at 25° C. The mixture was stirred at 25° C. for 2 hours. The reaction mixture was poured into water and extracted with ethyl acetate. The organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 1-(3-(pyridin-4-yl)-2-(4-((trifluoromethyl)thio)phenyl)-6,7-dihydropyrazolo[1,5-a]pyrazin-5(4H)-yl) prop-2-en-1-one (24.6 mg, 98% purity, 18% yield) as a colourless oil.

LC-MS (Method 22): Rₜ=0.957 min; MS (ESIpos): m/z=431.1 [M+H]⁺

¹H NMR (400 MHz, DMSO-d6) δ [ppm]=8.57 (s, 2H), 7.70 (d, J=8.0 Hz, 2H), 7.51 (d, J=8.0 Hz, 2H), 7.21 (s, 2H), 7.04-6.80 (m, 1H), 6.18 (d, J=16.0 Hz, 1H), 5.87-5.64 (m, 1H), 5.12-4.70 (m, 2H), 4.47-3.97 (m, 4H).

### Example 144

### (2E)-1-{2-[4-(difluoromethoxy)phenyl]-3-(pyridin-4-yl)-6,7-dihydropyrazolo[1,5-a]pyrazin-5(4H)-yl}-4-(dimethylamino)but-2-en-1-one

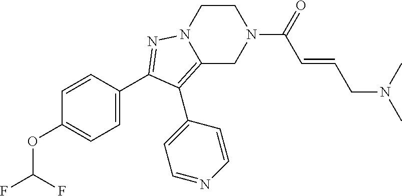 [SMILES: CN(C)C/C=C/C(=O)N1CCn2nc(-c3ccc(OC(F)F)cc3)c(-c3ccncc3)c2C1]

To a solution of 2-(4-(difluoromethoxy)phenyl)-3-(pyridin-4-yl)-4,5,6,7-tetrahydropyrazolo[1,5-a]pyrazine hydrochloride (1:1) (150 mg, 0.395 mmol, Intermediate 191) and (2E)-4-(dimethylamino)but-2-enoic acid hydrochloride (1:1) (98.3 mg, 0.593 mmol) in N,N-dimethylformamide (10 ml) were added 1-[bis(dimethylamino)methylene]-1H-1,2,3-triazolo[4,5-b]pyridinium-3-oxide hexafluorophosphate (225 mg, 0.593 mmol) and N,N-diisopropylethylamine (102 mg, 0.791 mmol) at 20° C. The mixture was stirred at 20° C. for 16 hours. The reaction mixture was poured into water and extracted withed ethyl acetate. The combined organic layers were washed with brine, dried over anhydrous sulfate, then filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 28%-58%, 9 min) to give (2E)-1-{2-[4-(difluoromethoxy)phenyl]-3-(pyridin-4-yl)-6,7-dihydropyrazolo[1,5-a]pyrazin-5(4H)-yl}-4-(dimethylamino)but-2-en-1-one (39.5 mg, 0.0857 mmol, 21% yield) as a white solid.

LC-MS (Method 20): Rₜ=0.869 min; MS (ESIpos): m/z=453.2 [M+H]⁺.

¹H NMR (400 MHz, DMSO-d6) δ [ppm]=8.55 (d, J=5.2 Hz, 2H), 7.44 (s, 1H), 7.39 (d, J=8.4 Hz, 2H), 7.26 (s, 1H), 7.20-7.13 (m, 4H), 7.07 (s, 1H), 6.79-6.58 (m, 2H), 4.97-4.77 (m, 2H), 4.27 (br s, 2H), 4.13 (br s, 2H), 3.05 (d, J=4.4 Hz, 2H), 2.22-2.03 (m, 6H).

### Example 145

### (2E)-4-(dimethylamino)-1-[3-(pyridin-4-yl)-2-[4-(trifluoromethoxy)phenyl]-6,7-dihydropyrazolo[1,5-a]pyrazin-5(4H)-yl]but-2-en-1-one

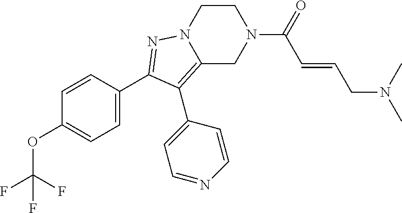 [SMILES: CN(C)C/C=C/C(=O)N1CCn2nc(-c3ccc(OC(F)(F)F)cc3)c(-c3ccncc3)c2C1]

To a solution of 3-(pyridin-4-yl)-2-[4-(trifluoromethoxy)phenyl]-4,5,6,7-tetrahydropyrazolo[1,5-a]pyrazine hydrochloride (1:1) (110 mg, 0.277 mmol, Intermediate 193) and (2E)-4-(dimethylamino)but-2-enoic acid hydrochloride (1:1) (68.8 mg, 0.415 mmol) in N,N-dimethylformamide (2.0 ml) were added 1-[bis(dimethylamino)methylene]-1H-1,2,3-triazolo[4,5-b]pyridinium-3-oxide hexafluorophosphate (158 mg, 0.415 mmol) and N,N-diisopropylethylamine (71.6 mg, 0.554 mmol) at 25° C. The mixture was stirred at 25° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 36%-66%, 9 min) to give (2E)-4-(dimethylamino)-1-[3-(pyridin-4-yl)-2-[4-(trifluoromethoxy)phenyl]-6,7-dihydropyrazolo[1,5-a]pyrazin-5(4H)-yl]but-2-en-1-one (25.8 mg, 0.0533 mmol, 19% yield) as a white solid.

LC-MS (Method 20): Rₜ=0.601 min; MS (ESIpos): m/z=471.1 [M+H]⁺.

1H NMR (400 MHz, DMSO-d6) δ [ppm]=8.56 (d, J=5.6 Hz, 2H), 7.46 (d, J=8.4 Hz, 2H), 7.35 (d, J=8.0 Hz, 2H), 7.18 (d, J=4.8 Hz, 2H), 6.82-6.58 (m, 2H), 5.01-4.75 (m, 2H), 4.28 (br s, 2H), 4.18-4.03 (m, 2H), 3.04 (br s, 2H), 2.20-2.05 (m, 6H).

### Example 146

### (2E)-4-(dimethylamino)-1-[2-(4-ethynylphenyl)-3-(pyridin-4-yl)-6,7-dihydropyrazolo[1,5-a]pyrazin-5(4H)-yl]but-2-en-1-one

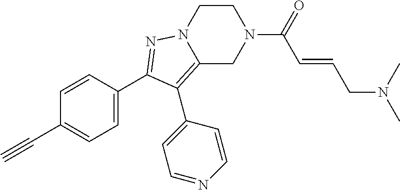 [SMILES: C#Cc1ccc(-c2nn3c(c2-c2ccncc2)CN(C(=O)/C=C/CN(C)C)CC3)cc1]

To a solution of 2-(4-ethynylphenyl)-3-(pyridin-4-yl)-4,5,6,7-tetrahydropyrazolo[1,5-a]pyrazine trifluoroacetate (1:1) (90.0 mg, 0.217 mmol, Intermediate 225) and (2E)-4-(dimethylamino)but-2-enoic acid hydrochloride (1:1) (53.9 mg, 0.325 mmol) in N,N-dimethylformamide (3 ml) were added 1-[bis(dimethylamino)methylene]-1H-1,2,3-triazolo[4,5-b]pyridinium-3-oxide hexafluorophosphate (123 mg, 0.325 mmol) and N,N-diisopropylethylamine (56.1 mg, 434 mmol) at 20° C. The mixture was stirred at 20° C. for 16 hours. The reaction mixture was poured into water and extracted with ethyl acetate. The combined organic layers were dried over anhydrous sodium sulfate, filtered and the filtrate was concentrated in vacuo to give a residue. The residue was purified by preparative HPLC (column: Phenomenex luna C18 150*25 mm*10 μm; mobile phase: water (FA)-ACN; B %: 0%-30%, 10 min) to give (2E)-4-(dimethylamino)-1-[2-(4-ethynylphenyl)-3-(pyridin-4-yl)-6,7-dihydropyrazolo[1,5-a]pyrazin-5(4H)-yl]but-2-en-1-one (3.00 mg, 0.00682 mmol, 3% yield) as a white solid.

LC-MS (Method 21): Rₜ=0.732 min; MS (ESIpos): m/z=411.2 [M+H]⁺.

¹H NMR (400 MHz, METHANOL-d4) δ [ppm]=8.51 (d, J=3.2 Hz, 2H), 7.51-7.44 (m, 1H), 7.40-7.40 (m, 1H), 7.43-7.37 (m, 3H), 7.28 (d, J=5.2 Hz, 2H), 7.05 (d, J=16.4 Hz, 1H), 6.95-6.74 (m, 2H), 6.53 (d, J=16.0 Hz, 1H), 5.06-4.99 (m, 1H), 4.41-4.29 (m, 2H), 4.23 (t, J=5.2 Hz, 2H), 3

### Example 147

### (2E)-4-(dimethylamino)-1-[2-(4-ethoxyphenyl)-3-(pyridin-4-yl)-6,7-dihydropyrazolo[1,5-a]pyrazin-5(4H)-yl]but-2-en-1-one

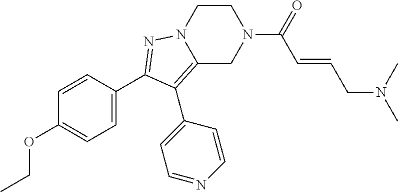 [SMILES: CCOc1ccc(-c2nn3c(c2-c2ccncc2)CN(C(=O)/C=C/CN(C)C)CC3)cc1]

To a solution of 2-(4-ethoxyphenyl)-3-(pyridin-4-yl)-4,5,6,7-tetrahydropyrazolo[1,5-a]pyrazine hydrochloride (1:1) (150 mg, 0.420 mmol, Intermediate 195) and (2E)-4-(dimethylamino)but-2-enoic acid hydrochloride (1:1) (104.4 mg, 0.630 mmol) in N,N-dimethylformamide (8.9 ml) were added 1-[bis(dimethylamino)methylene]-1H-1,2,3-triazolo[4,5-b]pyridinium-3-oxide hexafluorophosphate (239 mg, 0.630 mmol) and N,N-diisopropylethylamine (108 mg, 0.840 mmol) at 20° C. The mixture was stirred at 20° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 27%-57%, 9 min) to give (2E)-4-(dimethylamino)-1-[2-(4-ethoxyphenyl)-3-(pyridin-4-yl)-6,7-dihydropyrazolo[1,5-a]pyrazin-5(4H)-yl]but-2-en-1-one (81.1 mg, 0.181 mmol, 43% yield) as a brown oil.

LC-MS (Method 20): Rₜ=0.933 min; MS (ESIpos): m/z=431.2 [M+H]⁺.

1H NMR (400 MHz, DMSO-d6) δ [ppm]=8.52 (d, J=5.2 Hz, 2H), 7.25 (d, J=8.4 Hz, 2H), 7.15 (d, J=4.4 Hz, 2H), 6.90 (s, 1H), 6.88 (s, 1H), 6.82-6.58 (m, 2H), 4.97-4.76 (m, 3H), 4.25 (br s, 2H), 4.13 (br s, 2H), 4.01 (q, J=7.2 Hz, 2H), 3.05 (d, J=4.8 Hz, 3H), 2.20-2.05 (m, 6H), 1.31 (t, J=6.4 Hz, 3H).

### Example 148

### (2E)-1-{2-[4-(difluoromethyl)phenyl]-3-(pyridin-4-yl)-6,7-dihydropyrazolo[1,5-a]pyrazin-5(4H)-yl}-4-(dimethylamino)but-2-en-1-one

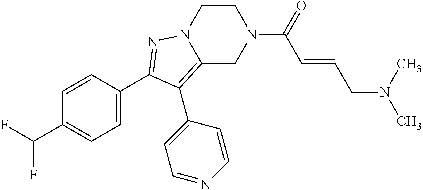 [SMILES: CN(C)C/C=C/C(=O)N1CCn2nc(-c3ccc(C(F)F)cc3)c(-c3ccncc3)c2C1]

To a solution of 2-[4-(difluoromethyl)phenyl]-3-(pyridin-4-yl)-4,5,6,7-tetrahydropyrazolo[1,5-a]pyrazine hydrochloride (1/1) (110 mg, 0.303 mmol, Intermediate 197) and (2E)-4-(dimethylamino)but-2-enoic acid hydrochloride (1/1) (75.3 mg, 0.455 mmol) in N,N-dimethylformamide (2.7 ml) were added 1-[bis(dimethylamino)methylene]-1H-1,2,3-triazolo[4,5-b]pyridinium-3-oxide hexafluorophosphate (173 mg, 0.455 mmol) and N,N-diisopropylethylamine (78.3 mg, 0.606 mmol) at 25° C. The mixture was stirred at 25° C. for 16 hours. The reaction mixture was poured into water and extracted with ethyl acetate. The combined organic layers were dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 25%-55%, 9 min) to give (2E)-1-{2-[4-(difluoromethyl)phenyl]-3-(pyridin-4-yl)-6,7-dihydropyrazolo[1,5-a]pyrazin-5(4H)-yl}-4-(dimethylamino)but-2-en-1-one (61.0 mg, 0.137 mmol, 45% yield) as a brown oil.

LC-MS (Method 21): Rₜ=0.933 min; MS (ESIpos): m/z=437.2 [M+H]⁺.

1H NMR (400 MHz, DMSO-d6) δ [ppm]=8.56 (d, J=5.6 Hz, 2H), 7.58-7.53 (m, 2H), 7.52-7.44 (m, 2H), 7.19 (br s, 2H), 7.18-7.17 (m, 1H), 7.04 (s, 1H), 6.90 (s, 1H), 6.83-6.57 (m, 2H), 4.99-4.79 (m, 2H), 4.30 (br s, 2H), 4.20-4.03 (m, 2H), 3.06 (d, J=5.2 Hz, 2H), 2.21-2.05 (m, 5H), 2.24-2.01 (m, 1H).

### Example 149

### (2E)-1-[2-(5-chloro-2-fluorophenyl)-3-(pyridin-4-yl)-6,7-dihydropyrazolo[1,5-a]pyrazin-5(4H)-yl]-4-(dimethylamino)but-2-en-1-one

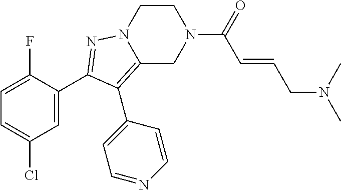 [SMILES: CN(C)C/C=C/C(=O)N1CCn2nc(-c3cc(Cl)ccc3F)c(-c3ccncc3)c2C1]

To a solution of 2-(5-chloro-2-fluorophenyl)-3-(pyridin-4-yl)-4,5,6,7-tetrahydropyrazolo[1,5-a]pyrazine hydrochloride (1:1) (150 mg, 0.410 mmol, Intermediate 199) and (2E)-4-(dimethylamino)but-2-enoic acid hydrochloride (1:1) (102 mg, 0.616 mmol) in N,N-dimethylformamide (9.0 ml) were added 1-[bis(dimethylamino)methylene]-1H-1,2,3-triazolo[4,5-b]pyridinium-3-oxide hexafluorophosphate (234 mg, 0.616 mmol) and N,N-diisopropylethylamine (106 mg, 0.821 mmol) at 25° C. The mixture was stirred at 25° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase water: (ammonia hydroxide v/v)-ACN; B %: 30%-60%, 9 min) to give (2E)-1-[2-(5-chloro-2-fluorophenyl)-3-(pyridin-4-yl)-6,7-dihydropyrazolo[1,5-a]pyrazin-5(4H)-yl]-4-(dimethylamino)but-2-en-1-one (38.0 mg, 0.0859 mmol, 21% yield) as an orange solid.

LC-MS (Method 20): Rₜ=0.545 min; MS (ESIpos): m/z=439.1 [M+H]⁺, 1H NMR (400 MHz, DMSO-d₆) δ [ppm]=8.51 (d, J=4.8 Hz, 2H), 7.53 (dd, J=5.6, 3.2 Hz, 2H), 7.27 (t, J=9.6 Hz, 1H), 7.10 (br s, 2H), 6.96-6.78 (m, 1H), 6.74-6.61 (m, 1H), 5.11-4.86 (m, 2H), 4.39-4.23 (m, 2H), 4.21-4.05 (m, 2H), 3.41 (br s, 2H), 2.44-2.29 (m, 6H).

### Example 150

### (2E)-1-[2-(3-chlorophenyl)-3-(pyridin-4-yl)-6,7-dihydropyrazolo[1,5-a]pyrazin-5(4H)-yl]-4-(dimethylamino)but-2-en-1-one

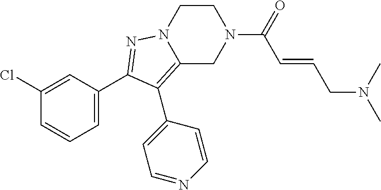 [SMILES: CN(C)C/C=C/C(=O)N1CCn2nc(-c3cccc(Cl)c3)c(-c3ccncc3)c2C1]

To a solution of 2-(3-chlorophenyl)-3-(pyridin-4-yl)-4,5,6,7-tetrahydropyrazolo[1,5-a]pyrazine hydrochloride (1:1) (100 mg, 0.287 mmol, Intermediate 201) and (2E)-4-(dimethylamino)but-2-enoic acid hydrochloride (1:1) (71.5 mg, 0.431 mmol) in N,N-dimethylformamide (2.0 ml) were added 1-[bis(dimethylamino)methylene]-1H-1,2,3-triazolo[4,5-b]pyridinium-3-oxide hexafluorophosphate (164 mg, 0.431 mmol) and N,N-diisopropylethylamine (74.4 mg, 0.575 mmol) at 25° C. The mixture was stirred at 25° C. for 16 hours. The reaction solution was poured into water and extracted with ethyl aceate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 30%-60%, 9 min) to give (2E)-1-[2-(3-chlorophenyl)-3-(pyridin-4-yl)-6,7-dihydropyrazolo[1,5-a]pyrazin-5(4H)-yl]-4-(dimethylamino)but-2-en-1-one (41.8 mg, 0.0964 mmol, 33% yield) as a white solid.

LC-MS (Method 20): Rₜ=0.933 min; MS (ESIpos): m/z=421.1 [M+H]⁺.

¹H NMR (400 MHz, DMSO-d₆) δ=8.57 (d, J=5.6 Hz, 2H), 7.43-7.35 (m, 3H), 7.26 (d, J=7.2 Hz, 1H), 7.20 (d, J=4.4 Hz, 2H), 6.82-6.58 (m, 2H), 4.99-4.78 (m, 2H), 4.29 (br s, 2H), 4.20-4.04 (m, 2H), 3.05 (br s, 2H), 2.24-1.97 (m, 6H).

### Example 151

### (2E)-4-(dimethylamino)-1-{2-[3-fluoro-4-(trifluoromethyl)phenyl]-3-(pyridin-4-yl)-6,7-dihydropyrazolo[1,5-a]pyrazin-5(4H)-yl}but-2-en-1-one

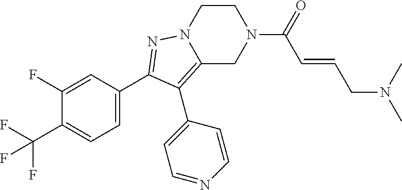 [SMILES: CN(C)C/C=C/C(=O)N1CCn2nc(-c3ccc(C(F)(F)F)c(F)c3)c(-c3ccncc3)c2C1]

To a solution of 2-[3-fluoro-4-(trifluoromethyl)phenyl]-3-(pyridin-4-yl)-4,5,6,7-tetrahydropyrazolo[1,5-a]pyrazine hydrochloride (1:1) (100 mg, 0.250 mmol, Intermediate 203) and (2E)-4-(dimethylamino)but-2-enoic acid hydrochloride (1:1) (62.2 mg, 0.376 mmol) in N,N-dimethylformamide (4.5 ml) were added 1-[bis(dimethylamino)methylene]-1H-1,2,3-triazolo[4,5-b]pyridinium-3-oxide hexafluorophosphate (143 mg, 0.376 mmol) and N,N-diisopropylethylamine (64.8 mg, 0.501 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 35%-65%, 9 min) to give (2E)-4-(dimethylamino)-1-{2-[3-fluoro-4-(trifluoromethyl)phenyl]-3-(pyridin-4-yl)-6,7-dihydropyrazolo[1,5-a]pyrazin-5(4H)-yl}but-2-en-1-one (62.0 mg, 97% purity, 127 μmol, 51% yield) as a yellow solid.

LC-MS (Method 20): Rₜ=0.998 min; MS (ESIpos): m/z=473.4 [M+H]⁺.

¹H NMR (400 MHz, DMSO-da) δ [ppm]=8.60 (d, J=5.2 Hz, 2H), 7.77 (t, J=8.0 Hz, 1H), 7.43 (d, J=11.6 Hz, 1H), 7.33 (d, J=8.0 Hz, 1H), 7.24 (d, J=4.4 Hz, 2H), 6.82-6.58 (m, 2H), 4.99-4.75 (m, 2H), 4.32 (br s, 2H), 4.20-4.06 (m, 2H), 3.04 (br s, 2H), 2.23-2.01 (m, 6H).

### Example 152

### (2E)-4-(dimethylamino)-1-{2-[2-fluoro-4-(trifluoromethyl)phenyl]-3-(pyridin-4-yl)-6,7-dihydropyrazolo[1,5-a]pyrazin-5(4H)-yl}but-2-en-1-one

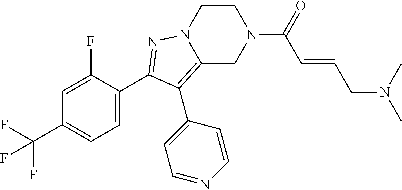 [SMILES: CN(C)C/C=C/C(=O)N1CCn2nc(-c3ccc(C(F)(F)F)cc3F)c(-c3ccncc3)c2C1]

To a solution of 2-[2-fluoro-4-(trifluoromethyl)phenyl]-3-(pyridin-4-yl)-4,5,6,7-tetrahydropyrazolo[1,5-a]pyrazine hydrochloride (1:1) (150 mg, 0.376 mmol, Intermediate 205) and (2E)-4-(dimethylamino)but-2-enoic acid hydrochloride (1:1) (93.4 mg, 0.564 mmol) in N,N-Dimethylformamide (2.7 ml) were added 1-[bis(dimethylamino)methylene]-1H-1,2,3-triazolo[4,5-b]pyridinium-3-oxide hexafluorophosphate (214 mg, 0.564 mmol) and N,N-diisopropylethylamine (97.2 mg, 0.752 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 32%-62%, 9 min) to give (2E)-4-(dimethylamino)-1-{2-[2-fluoro-4-(trifluoromethyl)phenyl]-3-(pyridin-4-yl)-6,7-dihydropyrazolo[1,5-a]pyrazin-5(4H)-yl}but-2-en-1-one (74.8 mg, 0.151 mmol, 40% yield) as a gray solid.

LC-MS (Method 20): Rₜ=0.974 min; MS (ESIpos): m/z=473.1 [M+H]⁺.

¹H NMR (400 MHz, DMSO-d₆) δ [ppm]=8.52 (d, J=5.6 Hz, 2H), 7.78-7.57 (m, 3H), 7.10 (d, J=3.6 Hz, 2H), 6.81-6.59 (m, 2H), 5.09-4.84 (m, 2H), 4.32 (br s, 2H), 4.21-4.06 (m, 2H), 3.05 (br s, 2H), 2.25-1.96 (m, 7H).

### Example 153

### (2E)-4-(dimethylamino)-1-[2-(2-fluorophenyl)-3-(pyridin-4-yl)-6,7-dihydropyrazolo[1,5-a]pyrazin-5(4H)-yl]but-2-en-1-one

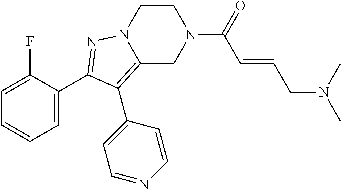 [SMILES: CN(C)C/C=C/C(=O)N1CCn2nc(-c3ccccc3F)c(-c3ccncc3)c2C1]

To a solution of (2E)-4-(dimethylamino)but-2-enoic acid hydrochloride (50.1 mg, 302 μmol) and N,N-diisopropylethylamine (156 mg, 1.21 mmol) in dichloromethane (6 ml) was added 1-[bis(dimethylamino)methylene]-1H-1,2,3-triazolo[4,5-b]pyridinium-3-oxide hexafluorophosphate (172 mg, 453 μmol) at 20° C. The mixture was stirred at 20° C. for 0.1 hour. Then to the solution above was added 2-(2-fluorophenyl)-3-(pyridin-4-yl)-4,5,6,7-tetrahydropyrazolo[1,5-a]pyrazine hydrochloride (100 mg, 302 μmol, Intermediate 207) and the resulting mixture was stirred at 20° C. for 2 hours. The reaction mixture was concentrated under vacuum to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 22-52% B; flow 25 ml/min; temperature: RT; Detector: UV 220/254 nm] to give (2E)-4-(dimethylamino)-1-[2-(2-fluorophenyl)-3-(pyridin-4-yl)-6,7-dihydropyrazolo[1,5-a]pyrazin-5(4H)-yl]but-2-en-1-one (28.8 mg, 68.9 μmol, 97% purity, 23% yield) as a grey solid.

LC-MS (Method 20): Rₜ=0.829 min; MS (ESIpos): m/z=406.1 [M+H]⁺.

¹H NMR (400 MHz, DMSO-d₆) δ [ppm]=8.48 (d, J=5.2 Hz, 2H), 7.53-7.40 (m, 2H), 7.34-7.25 (m, 1H), 7.19 (t, J=9.2 Hz, 1H), 7.06 (s, 2H), 6.83-6.60 (m, 2H), 5.14-4.82 (m, 2H), 4.45-3.99 (m, 4H), 3.05 (s, 2H), 2.24-1.99 (m, 6H).

### Example 154

### (2E)-1-[2-(2-chlorophenyl)-3-(pyridin-4-yl)-6,7-dihydropyrazolo[1,5-a]pyrazin-5(4H)-yl]-4-(dimethylamino)but-2-en-1-one

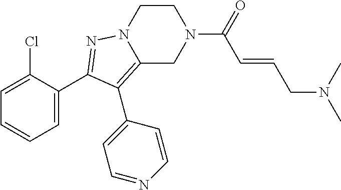 [SMILES: CN(C)C/C=C/C(=O)N1CCn2nc(-c3ccccc3Cl)c(-c3ccncc3)c2C1]

To a solution of (2E)-4-(dimethylamino)but-2-enoic acid hydrochloride (47.7 mg, 288 μmol) and N,N-diisopropylethylamine (149 mg, 1.15 mmol) in dichloromethane (5 ml) was added 1-[bis(dimethylamino)methylene]-1H-1,2,3-triazolo[4,5-b]pyridinium-3-oxide hexafluorophosphate (164 mg, 432 μmol) at 20° C. and the mixture was stirred at 20° C. for 0.1 hour. Then to the mixture above was added 2-(2-chlorophenyl)-3-(pyridin-4-yl)-4,5,6,7-tetrahydropyrazolo[1,5-a]pyrazine hydrochloride (100 mg, 288 μmol, Intermediate 209) and the resulting mixture was stirred at 20° C. for 2 hours. The reaction mixture was concentrated under vacuum to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 24-54% B; flow 25 ml/min; temperature: RT; Detector: UV 220/254 nm] to give (2E)-1-[2-(2-chlorophenyl)-3-(pyridin-4-yl)-6,7-dihydropyrazolo[1,5-a]pyrazin-5(4H)-yl]-4-(dimethylamino)but-2-en-1-one (49.8 mg, 116 μmol, 98% purity, 40% yield) as a grey solid.

LC-MS (Method 20): Rₜ=0.861 min; MS (ESIpos): m/z=422.0 [M+H]⁺

¹H NMR (400 MHz, DMSO-d6) δ [ppm]=8.45 (d, J=5.6 Hz, 2H), 7.56-7.27 (m, 4H), 6.98 (s, 2H), 6.85-6.51 (m, 2H), 5.24-4.79 (m, 2H), 4.44-3.97 (m, 4H), 3.05 (s, 2H), 2.26-1.89 (m, 6H).

### Example 155

### (2E)-4-(dimethylamino)-1-[2-(3-methoxyphenyl)-3-(pyridin-4-yl)-6,7-dihydropyrazolo[1,5-a]pyrazin-5(4H)-yl]but-2-en-1-one

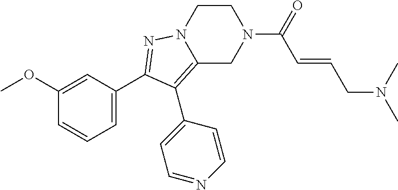

To a solution of (2E)-4-(dimethylamino)but-2-enoic acid hydrochloride (48.3 mg, 292 μmol) in dichloromethane (8 ml) were added 1-[bis(dimethylamino)methylene]-1H-1,2,3-triazolo[4,5-b]pyridinium-3-oxide hexafluorophosphate (166 mg, 438 μmol) and N,N-diisopropylethylamine (203 μl, 1.17 mmol) at 25° C. and the reaction mixture was stirred at 25° C. for 0.1 hour. Then to the mixture above was 2-(3-methoxyphenyl)-3-(pyridin-4-yl)-4,5,6,7-tetrahydropyrazolo[1,5-a]pyrazine hydrochloride (100 mg, 292 μmol, Intermediate 211 and the resulting mixture was stirred at 25° C. for 2 hours. The reaction mixture was concentrated under vacuum to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 24-54% B; flow 25 ml/min; temperature: RT; Detector: UV 220/254 nm] to give (2E)-4-(dimethylamino)-1-[2-(3-methoxyphenyl)-3-(pyridin-4-yl)-6,7-dihydropyrazolo[1,5-a]pyrazin-5(4H)-yl]but-2-en-1-one (26.2 mg, 61.5 μmol, 98% purity, 21% yield) as a grey solid.

LC-MS (Method 20): Rₜ=0.841 min; MS (ESIpos): m/z=418.1 [M+H]⁺.

¹H NMR (400 MHz, DMSO-d6) δ[ppm]=8.55 (d, J=5.6 Hz, 2H), 7.31-7.22 (m, 1H), 7.18 (d, J=4.4 Hz, 2H), 6.90 (d, J=6.0 Hz, 3H), 6.81-6.54 (m, 2H), 5.08-4.67 (m, 2H), 4.38-3.96 (m, 4H), 3.67 (s, 3H), 3.05 (d, J=4.0 Hz, 2H), 2.25-2.01 (m, 6H).

### Example 156

### (2E)-4-(dimethylamino)-1-[2-(4-methylphenyl)-3-(pyridin-4-yl)-6,7-dihydropyrazolo[1,5-a]pyrazin-5(4H)-yl]but-2-en-1-one

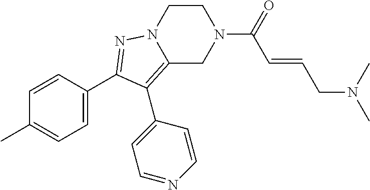

To a solution of (2E)-4-(dimethylamino)but-2-enoic acid hydrochloride (50.7 mg, 306 μmol) in dichloromethane (5 ml) were added 1-[bis(dimethylamino)methylene]-1H-1,2,3-triazolo[4,5-b]pyridinium-3-oxide hexafluorophosphate (175 mg, 459 μmol) and N,N-diisopropylethylamine (158 mg, 1.22 mmol) at 25° C. and the reaction mixture was stirred at 25° C. for 0.1 hour. Then to the mixture above was added 2-(4-methylphenyl)-3-(pyridin-4-yl)-4,5,6,7-tetrahydropyrazolo[1,5-a]pyrazine hydrochloride (100 mg, 306 μmol, Intermediate 213) and the resulting mixture was stirred at 25° C. for 2 hours. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 27-57% B; flow 25 ml/min; temperature: RT; Detector: UV 220/254 nm] to give (2E)-4-(dimethylamino)-1-[2-(4-methylphenyl)-3-(pyridin-4-yl)-6,7-dihydropyrazolo[1,5-a]pyrazin-5(4H)-yl]but-2-en-1-one (29.1 mg, 71.0 μmol, 98% purity, 23% yield) as a white solid.

LC-MS (Method 20): Rₜ=0.878 min; MS (ESIpos): m/z=402.2 [M+H]⁺.

¹H NMR (400 MHz, DMSO-d6) δ [ppm]=8.53 (d, J=5.6 Hz, 2H), 7.28-7.19 (m, 2H), 7.19-7.12 (m, 4H), 6.83-6.56 (m, 2H), 5.01-4.70 (m, 2H), 4.36-3.95 (m, 4H), 3.05 (d, J=4.8 Hz, 2H), 2.30 (s, 3H), 2.21-1.98 (m, 6H).

### Example 157

### (2E)-4-(dimethylamino)-1-[2-(3-methylphenyl)-3-(pyridin-4-yl)-6,7-dihydropyrazolo[1,5-a]pyrazin-5(4H)-yl]but-2-en-1-one

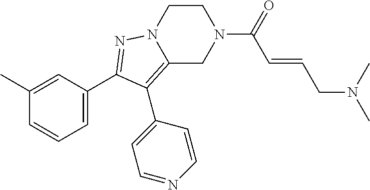

To a solution of (2E)-4-(dimethylamino)but-2-enoic acid hydrochloride (55.7 mg, 337 μmol) in dichloromethane (11 ml) were added 1-[bis(dimethylamino)methylene]-1H-1,2,3-triazolo[4,5-b]pyridinium-3-oxide hexafluorophosphate (192 mg, 505 μmol) and N,N-diisopropylethylamine (174 mg, 1.35 mmol) at 25° C. and the reaction mixture was stirred at 25° C. for 0.1 hour. Then to the mixture above was added 2-(3-methylphenyl)-3-(pyridin-4-yl)-4,5,6,7-tetrahydropyrazolo[1,5-a]pyrazine hydrochloride (110 mg, 337 μmol, Intermediate 215) and the resulting mixture was stirred at 25° C. for 2 hours. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 27-57% B; flow 25 ml/min; temperature: RT; Detector: UV 220/254 nm] to give (2E)-4-(dimethylamino)-1-[2-(3-methylphenyl)-3-(pyridin-4-yl)-6,7-dihydropyrazolo[1,5-a]pyrazin-5(4H)-yl]but-2-en-1-one (43.1 mg, 105 μmol, 98% purity, 31% yield) as a grey solid.

LC-MS (Method 20): Rₜ=0.885 min; MS (ESIpos): m/z=402.2 [M+H]⁺.

¹H NMR (400 MHz, DMSO-d6) δ [ppm]=8.53 (d, J=5.6 Hz, 2H), 7.29-7.11 (m, 5H), 7.05 (d, J=7.2 Hz, 1H), 6.81-6.56 (m, 2H), 5.02-4.74 (m, 2H), 4.34-4.00 (m, 4H), 3.04 (s, 2H), 2.26 (s, 3H), 2.20-2.04 (m, 6H).

### Example 158

### (2E)-4-(dimethylamino)-1-[2-(4-ethylphenyl)-3-(pyridin-4-yl)-6,7-dihydropyrazolo[1,5-a]pyrazin-5(4H)-yl]but-2-en-1-one

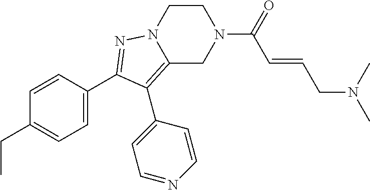 [SMILES: CCc1ccc(-c2nn3c(c2-c2ccncc2)CN(C(=O)/C=C/CN(C)C)CC3)cc1]

To a solution of 2-(4-ethylphenyl)-3-(pyridin-4-yl)-4,5,6,7-tetrahydropyrazolo[1,5-a]pyrazine hydrochloride (1:1) (100 mg, 0.293 mmol, Intermediate 217) and (E)-4-(dimethylamino)but-2-enoic acid hydrochloride (1:1) (72.8 mg, 0.440 mmol) in N,N-dimethylformamide (2.6 ml) were added 1-[bis(dimethylamino)methylene]-1H-1,2,3-triazolo[4,5-b]pyridinium-3-oxide hexafluorophosphate (167 mg, 0.440 mmol) and N,N-diisopropylethylamine (75.8 mg, 0.586 mmol) at 25° C. The mixture was stirred at 25° C. for 16 hours. The reaction mixture was poured into water and extracted with ethyl acetate. The combined organic layers were dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 30%-60%, 9 min) to give (2E)-4-(dimethylamino)-1-[2-(4-ethylphenyl)-3-(pyridin-4-yl)-6,7-dihydropyrazolo[1,5-a]pyrazin-5(4H)-yl]but-2-en-1-one (53.0 mg, 0.127 mmol, 43% yield) as brown oil.

LC-MS (Method 20): Rₜ=0.957 min; MS (ESIpos): m/z=415.2 [M+H]⁺.

¹H NMR (400 MHz, DMSO-d₆) δ [ppm]=8.54 (d, J=5.6 Hz, 2H), 7.27 (d, J=7.6 Hz, 2H), 7.19 (d, J=8.4 Hz, 4H), 6.81-6.60 (m, 2H), 5.07-4.71 (m, 2H), 4.27 (br s, 2H), 4.18-4.06 (m, 2H), 3.07 (d, J=3.6 Hz, 2H), 2.61 (q, J=7.6 Hz, 2H), 2.25-2.05 (m, 6H), 1.18 (t, J=7.6 Hz, 3H).

### Example 159

### 3-{5-[(2E)-4-(dimethylamino)but-2-enoyl]-3-(pyridin-4-yl)-4,5,6,7-tetrahydropyrazolo[1,5-a]pyrazin-2-yl}benzonitrile

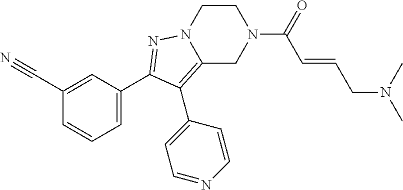 [SMILES: CN(C)C/C=C/C(=O)N1CCn2nc(-c3cccc(C#N)c3)c(-c3ccncc3)c2C1]

To a solution of (2E)-4-(dimethylamino)but-2-enoic acid hydrochloride (29.4 mg, 178 μmol) in dichloromethane (10 ml) were added 1-[bis(dimethylamino)methylene]-1H-1,2,3-triazolo[4,5-b]pyridinium-3-oxide hexafluorophosphate (101 mg, 266 μmol) and N,N-diisopropylethylamine (68.9 mg, 533 μmol) at 25° C. and the reaction mixture was stirred at 25° C. for 0.1 hour. Then to the mixture above was added 3-[3-(pyridin-4-yl)-4,5,6,7-tetrahydropyrazolo[1,5-a]pyrazin-2-yl]benzonitrile hydrochloride (60 mg, 178 μmol, Intermediate 219) and the resulting mixture was stirred at 25° C. for 2 hours. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 26-56% B; flow 25 ml/min; temperature: RT; Detector: UV 220/254 nm] to give 3-{5-[(2E)-4-(dimethylamino)but-2-enoyl]-3-(pyridin-4-yl)-4,5,6,7-tetrahydropyrazolo[1,5-a]pyrazin-2-yl}benzonitrile (17.3 mg, 40.7 μmol, 97% purity, 23% yield) as a grey solid.

LC-MS (Method 20): Rₜ=0.859 min; MS (ESIpos): m/z=413.2 [M+H]⁺.

¹H NMR (400 MHz, DMSO-d6) δ [ppm]=8.57 (d, J=5.6 Hz, 2H), 7.81 (dd, J=7.6, 1.2 Hz, 1H), 7.75 (s, 1H), 7.66-7.60 (m, 1H), 7.60-7.52 (m, 1H), 7.19 (d, J=4.4 Hz, 2H), 6.81-6.55 (m, 2H), 5.05-4.66 (m, 2H), 4.43-3.98 (m, 4H), 3.05 (s, 2H), 2.24-1.99 (m, 6H).

### Example 160

### (2E)-4-(dimethylamino)-1-[2-(2-fluoro-4-methoxyphenyl)-3-(pyridin-4-yl)-6,7-dihydropyrazolo[1,5-a]pyrazin-5(4H)-yl]but-2-en-1-one

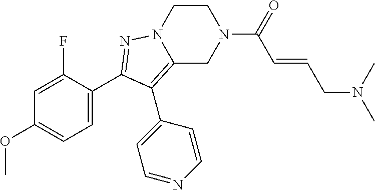 [SMILES: COc1ccc(-c2nn3c(c2-c2ccncc2)CN(C(=O)/C=C/CN(C)C)CC3)c(F)c1]

To a solution of (2E)-4-(dimethylamino)but-2-enoic acid hydrochloride (55.1 mg, 333 μmol) in dichlotomethane (10 ml) were added 1-[bis(dimethylamino)methylene]-1H-1,2,3-triazolo[4,5-b]pyridinium-3-oxide hexafluorophosphate (190 mg, 499 μmol) and N,N-diisopropylethylamine (172 mg, 1.33 mmol) at 25° C. and the reaction mixture was stirred at 25° C. for 0.1 hour. Then to the mixture above was added 2-(2-fluoro-4-methoxyphenyl)-3-(pyridin-4-yl)-4,5,6,7-tetrahydropyrazolo[1,5-a]pyrazine hydrochloride (120 mg, 333 μmol, Intermediate 221) and the resulting mixture was stirred at 25° C. for 2 hours. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 26-56% B; flow 25 ml/min; temperature: RT; Detector: UV 220/254 nm] to give (2E)-4-(dimethylamino)-1-[2-(2-fluoro-4-methoxyphenyl)-3-(pyridin-4-yl)-6,7-dihydropyrazolo[1,5-a]pyrazin-5(4H)-yl]but-2-en-1-one (47.6 mg, 106 μmol, 97% purity, 32% yield) as a grey solid.

LC-MS (Method 20): Rₜ=0.864 min; MS (ESIpos): m/z=436.2 [M+H]⁺.

¹H NMR (400 MHz, DMSO-d6) δ [ppm]=8.48 (d, J=5.6 Hz, 2H), 7.37 (t, J=8.8 Hz, 1H), 7.06 (d, J=3.6 Hz, 2H), 6.89-6.79 (m, 2H), 6.77-6.60 (m, 2H), 5.15-4.74 (m, 2H), 4.40-4.01 (m, 4H), 3.79 (s, 3H), 3.05 (s, 2H), 2.24-1.96 (m, 6H).

### Example 161

### (2E)-4-(dimethylamino)-1-[3-(pyridin-4-yl)-2-{4-[(trifluoromethyl) sulfanyl]phenyl}-6,7-dihydropyrazolo[1,5-a]pyrazin-5(4H)-yl]but-2-en-1-one

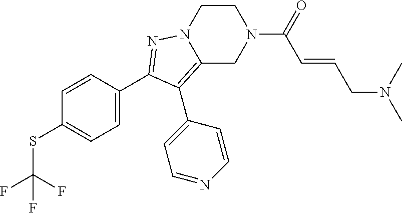 [SMILES: CN(C)C/C=C/C(=O)N1CCn2nc(-c3ccc(SC(F)(F)F)cc3)c(-c3ccncc3)c2C1]

To a solution of 3-(pyridin-4-yl)-2-(4-((trifluoromethyl)thio)phenyl)-4,5,6,7-tetrahydropyrazolo[1,5-a]pyrazine hydrochloride (130 mg, 86% purity, 0.290 mmol Intermediate 223) and (E)-4-(dimethylamino)but-2-enoic acid (36.0 mg, 0.280 mmol) in N,N-dimethylformamide (2 ml) were added O-(7-azabenzotriazol-1-yl)-N,N,N,N-tetramethyl uronium hexafluorophosphate (147 mg, 0.380 mmol) and N,N-diisopropylethylamine (0.2 ml, 10.3 mmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give (E)-4-(dimethylamino)-1-(3-(pyridin-4-yl)-2-(4-((trifluoromethyl)thio)phenyl)-6,7-dihydropyrazolo[1,5-a]pyrazin-5(4H)-yl) but-2-en-1-one (35.4 mg, 96% purity, 27% yield) as a colourless oil.

LC-MS (Method 22): Rₜ=0.955 min; MS (ESIpos): m/z=488.1 [M+H]⁺.

¹H NMR (400 MHz, DMSO-d6) δ [ppm]=8.57 (d, J=5.2 Hz, 2H), 7.7 (d, J=8.0 Hz, 2H), 7.5 (d, J=8.0 Hz, 2H), 7.2 (d, J=4.0 Hz, 2H), 6.46-6.88 (m, 2H), 5.02-4.70 (m, 2H), 4.40-3.97 (m, 4H), 3.05 (d, J=4.8 Hz, 2H), 2.23-2.02 (m, 6H).

### Example 162

### (2E)-4-(azetidin-1-yl)-1-[2-(4-chlorophenyl)-3-(pyridin-4-yl)-6,7-dihydropyrazolo[1,5-a]pyrazin-5(4H)-yl]but-2-en-1-one

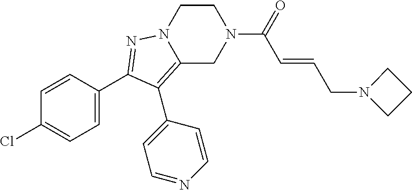 [SMILES: O=C(/C=C/CN1CCC1)N1CCn2nc(-c3ccc(Cl)cc3)c(-c3ccncc3)c2C1]

To a solution of 2-(4-chlorophenyl)-3-(pyridin-4-yl)-4,5,6,7-tetrahydropyrazolo[1,5-a]pyrazine (68.8 mg, 0.221 mmol, Intermediate 233) and (2E)-4-(azetidin-1-yl) but-2-enoic acid (250 mg, 0.885 mmol, Intermediate 227) in N,N-dimethylformamide (8 ml) were added 1-[bis(dimethylamino)methylene]-1H-1,2,3-triazolo[4,5-b]pyridinium 3-oxide hexafluorophosphate (84.2 mg, 0.221 mmol) and N,N-diisopropylethylamine (57.2 mg, 0.443 mmol) at 20° C. The mixture was stirred at 20° C. for 15 hours. The reaction mixture was poured into water and extracted with a mixed solvent of propan-2-ol and dichloromethane (1:3, v/v). The combined organic layers were washed with brine (20 ml), dried over anhydrous sodium sulfate, filtered and concentrated under reduced pressure to give a residue. The residue was purified by preparative HPLC [Instrument: ACSWH-GX-E; Column: Waters xbridge 150*25 mm 10 μm; eluent A: water (NH4HCO3)-ACN), eluent B: acetonitrile; gradient: 0-10 min 20-50% B; flow 29 ml/min; temperature: RT; Detector: UV 220/254 nm] to give (2E)-4-(azetidin-1-yl)-1-[2-(4-chlorophenyl)-3-(pyridin-4-yl)-6,7-dihydropyrazolo[1,5-a]pyrazin-5(4H)-yl]but-2-en-1-one (5.00 mg, 0.0111 mmol, 97% purity, 5% yield) as a white solid.

LC-MS (Method 20): Rₜ=0.995 min; MS (ESIpos): m/z=434.1 [M+H]⁺.

¹H NMR (400 MHz, DMSO-d₆), δ [ppm]=8.56 (d, J=6.0 Hz, 2H), 7.46-7.40 (m, 2H), 7.39-7.33 (m, 2H), 7.23-7.12 (m, 2H), 6.70-6.47 (m, 2H), 4.95-4.76 (m, 2H), 4.35-4.18 (m, 2H), 4.16-4.02 (m, 2H), 3.19-2.98 (m, 6H), 2.06-1.86 (m, 2H).

### Example 163

### (2E)-1-[2-(4-chlorophenyl)-3-(pyridin-4-yl)-6,7-dihydropyrazolo[1,5-a]pyrazin-5(4H)-yl]-4-(piperidin-1-yl) but-2-en-1-one

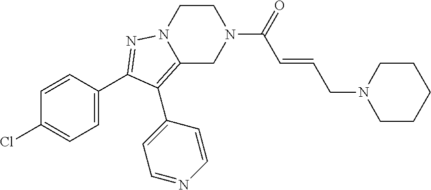 [SMILES: O=C(/C=C/CN1CCCCC1)N1CCn2nc(-c3ccc(Cl)cc3)c(-c3ccncc3)c2C1]

To a solution of 2-(4-chlorophenyl)-3-(pyridin-4-yl)-4,5,6,7-tetrahydropyrazolo[1,5-a]pyrazine (191.0 mg, 0.96 mmol, Intermediate 233) and (2E)-4-(piperidin-1-yl) but-2-enoic acid (131 mg, 0.772 mmol, Intermediate 229) in N,N-dimethylformamide (3 ml) were added N,N-diisopropylethylamine (125 mg, 0.965 mmol) and butylphosphonic anhydride (294 mg, 0.386 mmol) at 20° C. The mixture was stirred at 20° C. for 15 hours. The reaction mixture was poured into water (20 ml) and extracted with a mixed solvent of propan-2-ol and dichloromethane (1:3, v/v). The combined organic layers were washed with brine, dried over anhydrous sodium sulfate, filtered and concentrated under reduced pressure to give a residue. The residue was purified by preparative HPLC [Instrument: ACSWH-GX-E; Column: Waters xbridge 150*25 mm 10 μm; eluent A: water (NH4HCO3)-ACN), eluent B: acetonitrile; gradient: 0-10 min 20-50% B; flow 29 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-(piperidin-1-yl) but-2-en-1-one (35.3 mg, 0.0756 mmol, 99% purity, 39% yield) as a white solid.

LC-MS (Method 20): Rₜ=0.993 min; MS (ESIpos): m/z=462.1 [M+H]⁺.

¹H NMR (400 MHz, DMSO-da), δ [ppm]=8.60-8.52 (m, 2H), 7.46 (d, J=3.6 Hz, 2H), 7.38-7.32 (m, 2H), 7.23-7.13 (m, 2H), 6.80-6.58 (m, 2H), 4.99-4.74 (m, 2H), 4.33 (d, J=1.6 Hz, 2H), 4.18-4.03 (m, 2H), 3.14-2.98 (m, 2H), 2.36-2.24 (m, 4H), 1.59-1.31 (m, 6H).

### Example 164

### (2E)-1-[2-(4-chlorophenyl)-3-(pyridin-4-yl)-6,7-dihydropyrazolo[1,5-a]pyrazin-5(4H)-yl]-4-(4-methylpiperazin-1-yl) but-2-en-1-one

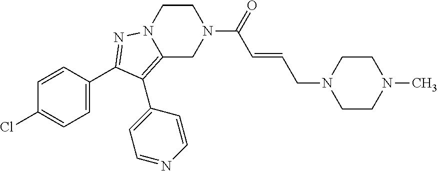 [SMILES: CN1CCN(C/C=C/C(=O)N2CCn3nc(-c4ccc(Cl)cc4)c(-c4ccncc4)c3C2)CC1]

To a solution of 2-(4-chlorophenyl)-3-(pyridin-4-yl)-4,5,6,7-tetrahydropyrazolo[1,5-a]pyrazine hydrochloride (1:1) (60.0 mg, 0.173 mmol, Intermediate 233) and (2E)-4-(4-methylpiperazin-1-yl) but-2-enoic acid (127 mg, 0.691 mmol, Intermediate 231) in N,N-dimethylformamide (2 ml) were added N,N-diisopropylethylamine (112 mg, 0.864 mmol) and butylphosphonic anhydride (263 mg, 0.346 mmol, 50% purity in ethyl acetate) at 20° C. The mixture was stirred at 20° C. for 15 hours. The reaction mixture was poured into water and extracted with a mixed solvent of dichloromethane and propan-2-ol (3:1, v/v). The combined organic layers were washed with brine, dried over anhydrous sodium sulfate, filtered and concentrated under reduced pressure to give a residue. The residue was purified by preparative HPLC [Instrument: ACSWH-GX-E; Column: Waters xbridge 150*25 mm 10 μm; eluent A: water (NH4HCO3)), eluent B: acetonitrile; gradient: 0-10 min 20-50% B; flow 29 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-(4-methylpiperazin-1-yl) but-2-en-1-one (12.6 mg, 0.0263 mmol, 99% purity, 15% yield) as a white solid.

LC-MS (Method 20): Rₜ=0.869 min; MS (ESIpos): m/z=477.2 [M+H]⁺.

¹H NMR (400 MHz, DMSO-d6), δ [ppm]=8.56 (d, J=4.4 Hz, 2H), 7.48-7.30 (m, 4H), 7.23-7.12 (m, 2H), 6.84-6.55 (m, 2H), 5.00-4.72 (m, 2H), 4.34-3.97 (m, 4H), 3.15-2.98 (m, 2H), 2.45-2.17 (m, 5H), 2.14 (s, 3H).

### Example 165

### (E)-1-(2-(4-chlorophenyl)-3-(pyridin-4-yl)-6,7-dihydropyrazolo[1,5-a]pyrazin-5(4H)-yl)-4-morpholinobut-2-en-1-one

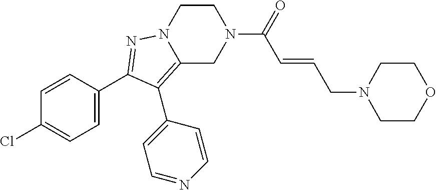 [SMILES: O=C(/C=C/CN1CCOCC1)N1CCn2nc(-c3ccc(Cl)cc3)c(-c3ccncc3)c2C1]

To a solution of (2E)-4-bromobut-2-enoic acid (107 mg, 0.648 mmol) and triethylamine (175 mg, 1.73 mmol) in tetrahydrofuran (4 ml) was added morpholine (56.5 mg, 0.648 mmol) at 20° C. and the mixture was stirred at 20° C. for 16 hours. To the mixture above were added N,N-dimethylformamide (4 ml), [dimethylamino (triazolo[4,5-b]pyridin-3-yloxy)methylidene]-dimethylazanium; hexafluorophosphate (197 mg, 0.518 mmol) and 2-(4-chlorophenyl)-3-(pyridin-4-yl)-4,5,6,7-tetrahydropyrazolo[1,5-a]pyrazine hydrochloride (1:1) (150 mg, 0.432 mmol, Intermediate 233) at 20° C. The resulting mixture was stirred at 20° C. for 4 hours. The reaction mixture was poured into water and extracted with a mixed solvent of dichloromethane and propan-2-ol (3:1, v/v). The combined organic layers were washed with brine, dried over sodium sulfate, filtered and the filtrate was concentrated under reduced pressure to give a residue. The residue was purified by preparative HPLC [Instrument: ACSWH-GX-D; Column: Waters Xbridge 150*25 mm*5 μm; eluent A: water (NH₄HCO₃), eluent B: acetonitrile; gradient: 0-10 min 24-54% B; flow 30 ml/min; temperature: RT; Detector: UV 220/254 nm] to give (E)-1-(2-(4-chlorophenyl)-3-(pyridin-4-yl)-6,7-dihydropyrazolo[1,5-a]pyrazin-5(4H)-yl)-4-morpholinobut-2-en-1-one (94.6 mg, 0.192 mmol, 94% purity, 45% yield) as an off-white solid.

LC-MS (Method 20): Rₜ=0.857 min; MS (ESIpos): m/z=464.3 [M+H]⁺.

¹H NMR (400 MHz, CDCl₃), δ [ppm]=8.64-8.54 (m, 2H), 7.37-7.33 (m, 2H), 7.32-7.28 (m, 2H), 7.10 (d, J=5.6 Hz, 2H), 7.00-6.89 (m, 1H), 6.40 (s, 1H), 4.98-4.80 (m, 2H), 4.45-4.30 (m, 2H), 4.24-4.05 (m, 2H), 3.57 (s, 4H), 3.36-3.16 (m, 2H), 2.77-2.33 (m, 4H).

### Example 166

### (2E)-1-[2-(4-chlorophenyl)-3-(pyridin-4-yl)-6,7-dihydropyrazolo[1,5-a]pyrazin-5(4H)-yl]-4-[isopropyl(methyl)amino]but-2-en-1-one

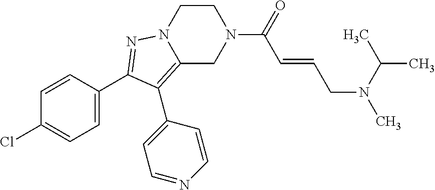 [SMILES: CC(C)N(C)C/C=C/C(=O)N1CCn2nc(-c3ccc(Cl)cc3)c(-c3ccncc3)c2C1]

To a solution of 2-(4-chlorophenyl)-3-(pyridin-4-yl)-4,5,6,7-tetrahydropyrazolo[1,5-a]pyrazine (80.0 mg, 0.257 mmol, Intermediate 233) and (2E)-4-[isopropyl(methyl)amino]but-2-enoic acid (80.9 mg, 0.515 mmol, Intermediate 235) in N,N-dimethylformamide (8 ml) were added 1-[bis(dimethylamino)methylene]-1H-1,2,3-triazolo[4,5-b]pyridinium 3-oxide hexafluorophosphate (117 mg, 0.309 mmol) and N,N-diisopropylethylamine (66.5 mg, 0.515 mmol) at 20° C. The mixture was stirred at 20° C. for 15 hours. The reaction mixture was poured into water and extracted with a mixed solvent of dichloromethane and propan-2-ol (3:1, v/v). The combined organic layers were washed with brine, dried over sodium sulfate, filtered and the filtrate was concentrated under reduced pressure to give a residue. The residue was purified by preparative HPLC [Instrument: Gilson-281; Column: Boston Green ODS 150*30*5 μm; eluent A: water (0.225% formic acid), eluent B: acetonitrile; gradient: 0-5 min 20-50% B; flow 25 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-[isopropyl(methyl)amino]but-2-en-1-one (45.1 mg, 0.0972 mmol, 97% purity, 38% yield) as a brown solid.

LC-MS (Method 20): Rₜ=0.990 min; MS (ESIpos): m/z=450.2 [M+H]⁺.

¹H NMR (400 MHz, DMSO-d6), δ [ppm]=8.56 (d, J=4.8 Hz, 2H), 7.45-7.40 (m, 2H), 7.38-7.33 (m, 2H), 7.17 (d, J=4.4 Hz, 2H), 6.81-6.58 (m, 2H), 4.97-4.74 (m, 2H), 4.35-4.19 (m, 2H), 4.17-4.02 (m, 2H), 3.19-3.06 (m, 2H), 2.82-2.70 (m, 1H), 2.13-1.99 (m, 3H), 1.02-0.79 (m, 6H).

### Example 167

### (2E)-4-(tert-butylamino)-1-[2-(4-chlorophenyl)-3-(pyridin-4-yl)-6,7-dihydropyrazolo[1,5-a]pyrazin-5(4H)-yl]but-2-en-1-one

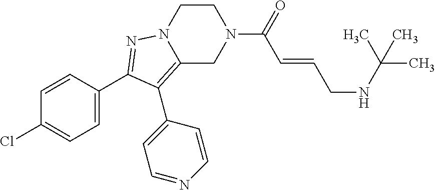 [SMILES: CC(C)(C)NC/C=C/C(=O)N1CCn2nc(-c3ccc(Cl)cc3)c(-c3ccncc3)c2C1]

To a solution of 2-(4-chlorophenyl)-3-(pyridin-4-yl)-4,5,6,7-tetrahydropyrazolo[1,5-a]pyrazine hydrochloride (1:1) (60.0 mg, 0.173 mmol, Intermediate 233) and (2E)-4-(tert-butylamino)but-2-enoic acid (54.3 mg, 0.346 mmol, Intermediate 237) in N,N-dimethylformamide (2 ml) were added N,N-diisopropylethylamine (112 mg, 0.864 mmol) and butylphosphonic anhydride (263 mg, 0.346 mmol, 50% purity in ethyl acetate) at 20° C. The mixture was stirred at 20° C. for 15 hours. The mixture was purified by preparative HPLC [Instrument: ACSWH-GX-E; Column: Waters Xbridge 150*25 mm*5 μm; eluent A: water (ammonia hydroxide v/v), eluent B: acetonitrile; gradient: 0-10 min 38-68% B; flow 25 ml/min; temperature: RT; Detector: UV 220/254 nm] to give (2E)-4-(tert-butylamino)-1-[2-(4-chlorophenyl)-3-(pyridin-4-yl)-6,7-dihydropyrazolo[1,5-a]pyrazin-5(4H)-yl]but-2-en-1-one (31.9 mg, 0.0674 mmol, 95% purity, 39% yield) as a white solid.

LC-MS (Method 20): Rₜ=0.996 min; MS (ESIpos): m/z=450.2 [M+H]⁺.

¹H NMR (400 MHz, DMSO-d6), δ [ppm]=8.56 (d, J=5.6 Hz, 2H), 7.46-7.39 (m, 2H), 7.39-7.32 (m, 2H), 7.22-7.12 (m, 2H), 6.84-6.53 (m, 2H), 5.03-4.65 (m, 2H), 4.34-4.19 (m, 2H), 4.17-4.00 (m, 2H), 3.30 (s, 2H), 1.04 (s, 9H).

### Example 168

### (2E)-1-[2-(4-chlorophenyl)-3-(pyridin-4-yl)-6,7-dihydropyrazolo[1,5-a]pyrazin-5(4H)-yl]-4-(4-methoxypiperidin-1-yl) but-2-en-1-one

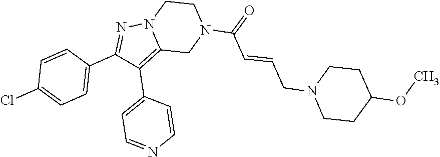 [SMILES: COC1CCN(C/C=C/C(=O)N2CCn3nc(-c4ccc(Cl)cc4)c(-c4ccncc4)c3C2)CC1]

To a solution of (2E)-4-bromobut-2-enoic acid (95.6 mg, 0.579 mmol) and 4-methoxypiperidine (66.7 mg, 0.579 mmol) in tetrahydrofuran (4 ml) were added triethylamine (117 mg, 1.16 mmol) at 20° C. After stirring at 20° C. for 3 hours, to the mixture above were added 1-[bis(dimethylamino)methylene]-1H-1,2,3-triazolo[4,5-b]pyridinium 3-oxide hexafluorophosphate (176 mg, 0.463 mmol) and 2-(4-chlorophenyl)-3-(pyridin-4-yl)-4,5,6,7-tetrahydropyrazolo[1,5-a]pyrazine (120 mg, 0.386 mmol, Intermediate 233) at 20° C. and the mixture was stirred at 20° C. for 15 hours. The reaction mixture was poured into water and extracted with a mixed solvent of dichloromethane and 2-propanol (3:1, v/v). The combined organic layers were washed with brine (30 ml), dried over anhydrous sodium sulfate, filtered and concentrated under reduced pressure to give a residue. The residue was purified by preparative HPLC [Instrument: Gilson-281; Column: Boston Green ODS 150*30*5 μm; eluent A: water (0.225% formic acid), eluent B: acetonitrile; gradient: 0-5 min 20-50% B; flow 25 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-(4-methoxypiperidin-1-yl) but-2-en-1-one (60.0 mg, 0.121 mmol, 99% purity, 31% yield) as a brown solid.

LC-MS (Method 20): Rₜ=0.925 min; MS (ESIpos): m/z=492.2 [M+H]⁺.

¹H NMR (400 MHz, DMSO-d₆), δ [ppm]=8.56 (d, J=5.2 Hz, 2H), 7.45-7.39 (m, 2H), 7.38-7.33 (m, 2H), 7.18 (br s, 2H), 6.79-6.60 (m, 2H), 5.02-4.67 (m, 2H), 4.33-4.17 (m, 2H), 4.16-4.02 (m, 2H), 3.21 (s, 3H), 3.18-3.02 (m, 3H), 2.71-2.55 (m, 2H), 2.16-1.98 (m, 2H), 1.89-1.66 (m, 2H), 1.50-1.29 (m, 2H).

### Example 169

### (2E)-1-[2-(4-chlorophenyl)-3-(pyridin-4-yl)-6,7-dihydropyrazolo[1,5-a]pyrazin-5(4H)-yl]-4-[(2-methoxyethyl)(methyl)amino]but-2-en-1-one

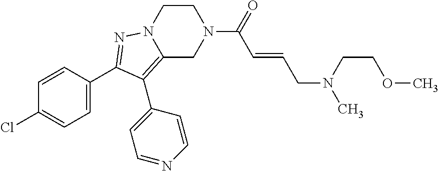 [SMILES: COCCN(C)C/C=C/C(=O)N1CCn2nc(-c3ccc(Cl)cc3)c(-c3ccncc3)c2C1]

To a solution of (2E)-4-bromobut-2-enoic acid (95.6 mg, 0.579 mmol) and 2-methoxy-N-methylethanamine (51.6 mg, 0.579 mmol) in tetrahydrofuran (4 ml) were added triethylamine (117 mg, 1.16 mmol) at 20° C. After stirring at 20° C. for 3 hours, to the mixture above were added 1-[bis(dimethylamino)methylene]-1H-1,2,3-triazolo[4,5-b]pyridinium 3-oxide hexafluorophosphate (176 mg, 0.463 mmol) and 2-(4-chlorophenyl)-3-(pyridin-4-yl)-4,5,6,7-tetrahydropyrazolo[1,5-a]pyrazine (120 mg, 0.386 mmol, Intermediate 233) at 20° C. and the mixture was stirred at 20° C. for 15 hours. The reaction mixture was poured into water (25 ml) and extracted with a mixed solvent of dichloromethane and 2-propanol (3:1, v/v). The combined organic layers were washed with brine (30 ml), dried over anhydrous sodium sulfate, filtered and concentrated under reduced pressure to give a residue. The residue was purified by preparative HPLC [Instrument: Gilson-281; Column: Boston Green ODS 150*30*5 μm; eluent A: water (0.225% formic acid), eluent B: acetonitrile; gradient: 0-5 min 20-50% B; flow 25 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-[(2-methoxyethyl)(methyl)amino]but-2-en-1-one (66.6 mg, 0.138 mmol, 97% purity, 36% yield) as a brown solid.

LC-MS (Method 20): Rₜ=0.907 min; MS (ESIpos): m/z=466.1 [M+H]⁺.

¹H NMR (400 MHz, DMSO-d6), δ [ppm]=8.56 (d, J=6.0 Hz, 2H), 7.46-7.40 (m, 2H), 7.38-7.32 (m, 2H), 7.17 (d, J=4.0 Hz, 2H), 6.83-6.60 (m, 2H), 5.00-4.76 (m, 2H), 4.36-4.21 (m, 2H), 4.14 (br s, 2H), 3.46-3.36 (m, 2H), 3.24 (br s, 2H), 3.16 (d, J=6.0 Hz, 3H), 2.47-2.38 (m, 2H), 2.23-2.07 (m, 3H).

### Example 170

### (2E)-1-[2-(4-chlorophenyl)-3-(pyridin-4-yl)-6,7-dihydropyrazolo[1,5-a]pyrazin-5(4H)-yl]-4-[(2-hydroxyethyl)(methyl)amino]but-2-en-1-one

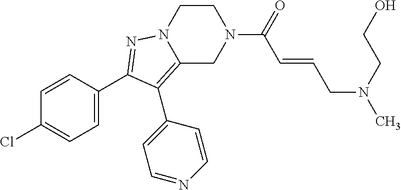 [SMILES: CN(C/C=C/C(=O)N1CCn2nc(-c3ccc(Cl)cc3)c(-c3ccncc3)c2C1)CCO]

To a solution of 2-(4-chlorophenyl)-3-(pyridin-4-yl)-4,5,6,7-tetrahydropyrazolo[1,5-a]pyrazine hydrochloride (1:1) (80.0 mg, 0.230 mmol, Intermediate 233) and (2E)-4-[(2-hydroxyethyl)(methyl)amino]but-2-enoic acid (110 mg, 0.691 mmol, Intermediate 240) in N,N-dimethylformamide (2 ml) were added N,N-diisopropylethylamine (149 mg, 1.15 mmol) and butylphosphonic anhydride (337 mg, 0.461 mmol, 50% purity in ethyl acetate) at 20° C. The mixture was stirred at 20° C. for 3.5 hours. The reaction mixture was concentrated under reduced pressure to give a residue. The residue was purified by preparative HPLC [Instrument: ACSWH-GX-H; Column: Phenomenex luna C18 150*40 mm*15 μm; eluent A: water (NH₄HCO₃)-ACN), eluent B: acetonitrile; gradient: 0-15 min 10-40% B; flow 60 ml/min; temperature: RT; Detector: UV 220/254 nm] to give a crude product. The crude product was further purified by preparative HPLC [Instrument: ACSWH-GX-E; Column: Waters Xbridge 150*25 mm*5 μm; eluent A: water (ammonia hydroxide v/v) eluent B: acetonitrile; gradient: 0-10 min 21-51% B; flow 25 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-[(2-hydroxyethyl)(methyl)amino]but-2-en-1-one (17.6 mg, 0.0390 mmol, 97% purity, 16% yield) as a white solid.

LC-MS (Method 16): Rₜ=0.886 min; MS (ESIpos): m/z=1452.2 [M+H]⁺.

¹H NMR (400 MHz, DMSO-d₆), δ [ppm]=8.56 (d, J=4.8 Hz, 2H), 7.47-7.39 (m, 2H), 7.36 (d, J=7.2 Hz, 2H), 7.18 (s, 2H), 7.06-6.80 (m, 1H), 6.77-6.54 (m, 1H), 5.04-4.91 (m, 1H), 4.84 (s, 1H), 4.37-4.21 (m, 2H), 4.19-4.01 (m, 2H), 3.85-3.52 (m, 4H), 3.01-2.74 (m, 2H), 2.57-2.53 (m, 3H).

### Example 171

### (2E)-1-[2-(4-chlorophenyl)-3-(pyridin-4-yl)-6,7-dihydropyrazolo[1,5-a]pyrazin-5(4H)-yl]-4-(pyrolidin-1-yl)but-2-en-1-one

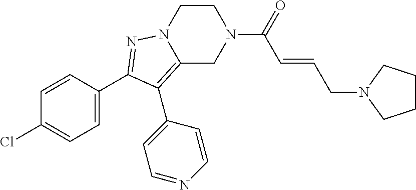 [SMILES: O=C(/C=C/CN1CCCC1)N1CCn2nc(-c3ccc(Cl)cc3)c(-c3ccncc3)c2C1]

To a solution of 2-(4-chlorophenyl)-3-(pyridin-4-yl)-4,5,6,7-tetrahydropyrazolo[1,5-a]pyrazine hydrochloride (1:1) (60.0 mg, 0.173 mmol, Intermediate 233) and (2E)-4-(pyrrolidin-1-yl) but-2-enoic acid (107 mg, 0.691 mmol, Intermediate 242) in N,N-dimethylformamide (2 ml) were added N,N-diisopropylethylamine (112 mg, 0.864 mml) and butylphosphonic anhydride (263 mg, 0.346 mmol, 50% purity in ethyl acetate) at 20° C. and the mixture was stirred at 20° C. for 15 hours. The reaction mixture was concentrated in vacuo to give a residue. The residue was purified by preparative HPLC [Instrument: ACSWH-GX-E; Column: Waters Xbridge 150*25 mm*5 μm; eluent A: water (ammonia hydroxide v/v) eluent B: acetonitrile; gradient: 0-10 min 29-50% B; flow 25 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-(pyrrolidin-1-yl) but-2-en-1-one (29.1 mg, 0.0630 mmol, 97% purity, 36% yield) as a white solid.

LC-MS (Method 20): Rₜ=0.942 min; MS (ESIpos): m/z=448.2 [M+H]⁺

¹H NMR (400 MHz, DMSO-d6), δ [ppm]=8.56 (d, J=4.4 Hz, 2H), 7.46-7.39 (m, 2H), 7.39-7.30 (m, 2H), 7.17 (d, J=3.2 Hz, 2H), 6.82-6.56 (m, 2H), 5.05-4.72 (m, 2H), 4.35-4.18 (m, 2H), 4.17-4.00 (m, 2H), 3.28-3.11 (m, 2H), 2.46-2.18 (m, 4H), 1.80-1.53 (m, 4H).

### Example 172

### (2E)-1-[2-(4-chlorophenyl)-3-(pyridin-4-yl)-6,7-dihydropyrazolo[1,5-a]pyrazin-5(4H)-yl]-4-(3-fluoroazetidin-1-yl) but-2-en-1-one

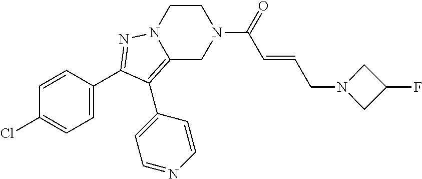 [SMILES: O=C(/C=C/CN1CC(F)C1)N1CCn2nc(-c3ccc(Cl)cc3)c(-c3ccncc3)c2C1]

To a solution of 2-(4-chlorophenyl)-3-(pyridin-4-yl)-4,5,6,7-tetrahydropyrazolo[1,5-a]pyrazine hydrochloride (1:1) (60.0 mg, 0.173 mmol, Intermediate 233) and (2E)-4-(3-fluoroazetidin-1-yl) but-2-enoic acid (165 mg, 1.04 mmol, Intermediate 244) in N,N-dimethylformamide (2 ml) were added N,N-diisopropylethylamine (156 mg, 12.1 mmol) and butylphosphonic anhydride (394 mg, 0.518 mmol, 50% purity in ethyl acetate) at 20° C. and the mixture was stirred at 20° C. for 15 hours. The reaction mixture was poured into water and extracted with a mixed solvent of propan-2-ol/dichloromethane (3:1, v/v). The combined organic layers were washed with brine, dried over anhydrous sodium sulfate, filtered and concentrated under reduced pressure to give a residue. The residue was was purified by preparative HPLC [Instrument: ACSWH-GX-E; Column: Waters Xbridge 150*25 mm*5 μm; eluent A: water (ammonia hydroxide v/v) eluent B: acetonitrile; gradient: 0-10 min 32-62% B; flow 25 ml/min; temperature: RT; Detector: UV 220/254 nm] to give (2E)-1-[2-(4-chlorophenyl)-3-(pyridin-4-yl)-6,7-dihydropyrazolo[1,5-a]pyrazin-5(4H)-yl]-4-(3-fluoroazetidin-1-yl) but-2-en-1-one (17.8 mg, 0.452 mmol, 97% purity, 22% yield) as a white solid.

LC-MS (Method 20): Rₜ=0.890 min; MS (ESIpos): m/z=452.2 [M+H]⁺.

¹H NMR (400 MHz, DMSO-d6), δ [ppm]=8.56 (d, J=6.0 Hz, 2H), 7.47-7.39 (m, 2H), 7.38-7.32 (m, 2H), 7.18 (s, 2H), 6.77-6.44 (m, 2H), 5.35-4.70 (m, 3H), 4.37-4.17 (m, 2H), 4.16-3.99 (m, 2H), 3.69-3.41 (m, 2H), 3.29-2.99 (m, 4H).

### Example 173

### 1-[2-(4-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one

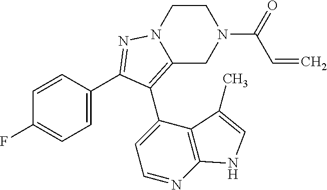 [SMILES: C=CC(=O)N1CCn2nc(-c3ccc(F)cc3)c(-c3ccnc4[nH]cc(C)c34)c2C1]

To a solution of 2-(4-fluorophenyl)-3-(3-methyl-1H-pyrrolo[2,3-b]pyridin-4-yl)-4,5,6,7-tetrahydropyrazolo[1,5-a]pyrazine hydrochloride (1:1) (45.0 mg, 0.117 mmol, Intermediate 248) and prop-2-enoyl chloride (10.7 mg, 0.117 mmol) in dichloromethane (8.0 ml) was added trimethylamine (0.0330 ml, 0.234 mmol) at 0° C. The mixture was stirred at 0° C. for 1 hour. The reaction mixture was concentrated in vacuo to give a residue. The residue was purified by preparative-HPLC (column: Phenomenex luna C18 150*25 mm*10 μm; mobile phase: water (TFA)-ACN; B %: 25%-40%, 10 min) to give 1-[2-(4-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one (10.4 mg, 0.0253 mmol, 97% purity, 22% yield) as a yellow solid.

LC-MS (Method 16): Rₜ=0.476 min; MS (ESIpos): m/z=402.1 [M+H]⁺.

¹H NMR (400 MHz, DMSO-d₆), δ [ppm]=11.45 (br s, 1H), 8.28-8.16 (m, 1H), 7.43-7.27 (m, 2H), 7.22-7.14 (m, 1H), 7.03 (t, J=8.4 Hz, 2H), 6.97-6.84 (m, 1H), 6.17 (d, J=17.2 Hz, 1H), 5.82-5.61 (m, 1H), 4.47 (br s, 2H), 4.38-4.27 (m, 2H), 4.20-4.06 (m, 2H), 1.87-1.53 (m, 3H).

### Example 174

### 1-(2-(2-fluoro-4-(trifluoromethyl)phenyl)-3-(3-methyl-1H-pyrrolo[2,3-b]pyridin-4-yl)-6,7-dihydropyrazolo[1,5-a]pyrazin-5(4H)-yl) prop-2-en-1-one

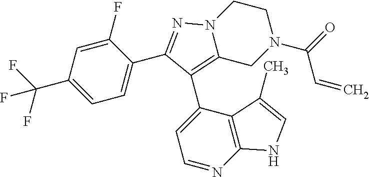 [SMILES: C=CC(=O)N1CCn2nc(-c3ccc(C(F)(F)F)cc3F)c(-c3ccnc4[nH]cc(C)c34)c2C1]

To a solution of 3-chloro-1-{2-[2-fluoro-4-(trifluoromethyl)phenyl]-3-(3-methyl-1H-pyrrolo[2,3-b]pyridin-4-yl)-6,7-dihydropyrazolo[1,5-a]pyrazin-5(4H)-yl}propan-1-one (45.0 mg, 0.0889 mmol, Intermediate 252) in acetonitrile (4.0 ml) was added trimethylamine (0.037 ml, 0.267 mmol) at 20° C. The mixture was stirred at 80° C. for 2 hours. The reaction mixture was concentrated in vacuo to give a residue. The residue was purified by preparative-HPLC (column: Phenomenex luna C18 150*25 mm*10 μm; mobile phase: water (TFA)-ACN; B %: 26%-56%, 10 min) to give 1-(2-(2-fluoro-4-(trifluoromethyl)phenyl)-3-(3-methyl-1H-pyrrolo[2,3-b]pyridin-4-yl)-6,7-dihydropyrazolo[1,5-a]pyrazin-5(4H)-yl) prop-2-en-1-one (18.5 mg, 0.0374 mmol, 95% purity, 42% yield) as a white solid.

LC-MS (Method 16): Rₜ=0.835 min; MS (ESIpos): m/z=470.2 [M+H]⁺

¹H NMR (400 MHz, DMSO-da), δ [ppm]=11.42 (br s, 1H), 8.13 (d, J=4.4 Hz, 1H), 7.65-7.43 (m, 3H), 7.17 (br s, 1H), 7.02-6.90 (m, 1H), 6.80-6.63 (m, 1H), 6.24-6.00 (m, 1H), 5.84-5.60 (m, 1H), 4.77-4.54 (m, 1H), 4.51-4.03 (m, 5H), 1.85-1.58 (m, 3H).

### Example 175

### 1-[2-(4-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one

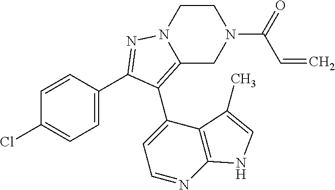 [SMILES: C=CC(=O)N1CCn2nc(-c3ccc(Cl)cc3)c(-c3ccnc4[nH]cc(C)c34)c2C1]

To a solution of 2-(4-chlorophenyl)-3-(3-methyl-1H-pyrrolo[2,3-b]pyridin-4-yl)-4,5,6,7-tetrahydropyrazolo[1,5-a]pyrazine trifluoroacetate (1:1) (80.0 mg, 0.167 mmol, Intermediate 257) and prop-2-enoic acid (18.1 mg, 0.251 mmol) in N,N-dimethylformamide (3.8 ml) was added 1-[bis(dimethylamino)methylene]-1H-1,2,3-triazolo[4,5-b]pyridinium-3-oxide hexafluorophosphate (95.5 mg, 0.251 mmol) and N,N-diisopropylethylamine (0.058 ml, 0.334 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 150*25 mm*10 μm; mobile phase: water (FA)-ACN; B %: 27%-57%, 10 min) to give 1-[2-(4-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one (9.00 mg, 0.0196 mmol, 12% yield, 91% purity) as a white solid.

LC-MS (Method 16): Rₜ=0.520 min; MS (ESIpos): m/z=418.2 [M+H]⁺.

¹H NMR (400 MHz, DMSO-d₆), δ [ppm]=11.60-11.36 (m, 1H), 8.27-8.12 (m, 1H), 7.38-7.13 (m, 5H), 7.07-6.77 (m, 2H), 6.25-6.03 (m, 1H), 5.85-5.60 (m, 1H), 4.59-4.38 (m, 2H), 4.37-4.24 (m, 2H), 4.21-4.03 (m, 2H), 1.70 (br s, 3H).

### Example 176

### 1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one

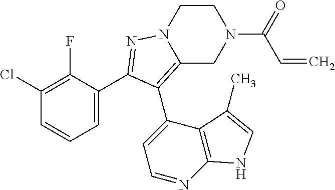 [SMILES: C=CC(=O)N1CCn2nc(-c3cccc(Cl)c3F)c(-c3ccnc4[nH]cc(C)c34)c2C1]

To a solution of 3-chloro-1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl]propan-1-one (150 mg, 0.318 mmol, Intermediate 261) in acetonitrile (8 ml) was added trimethylamine (96.4 mg, 0.953 mmol) at 20° C. The mixture was stirred at 80° C. for 12 hours. The reaction mixture was concentrated under reduced pressure to give a residue. The residue was purified by preparative HPLC [Instrument: Gilson-281; Column: Phenomenex luna C18 150*25 mm*10 μm; eluent A: water (0.225% formic acid), eluent B: acetonitrile; gradient: 0-25 min 22-52% B; flow 25 ml/min; temperature: RT; Detector: UV 220/254 nm] to give 1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one (30.0 mg, 0.662 mmol, 96% purity, 21% yield) as a white solid.

LC-MS (Method 16): Rₜ=0.511 min; MS (ESIpos): m/z=436.3 [M+H]⁺.

¹H NMR (400 MHz, DMSO-d6), δ [ppm]=11.43 (br s, 1H), 8.13 (d, J=4.8 Hz, 1H), 7.47 (t, J=7.2 Hz, 1H), 7.32-7.22 (m, 1H), 7.18 (br s, 1H), 7.14-7.07 (m, 1H), 7.04-6.76 (m, 1H), 6.74 (d, J=4.4 Hz, 1H), 6.17 (d, J=16.4 Hz, 1H), 5.78 (d, J=10.4 Hz, 1H), 4.75-4.52 (m, 1H), 4.51-4.41 (m, 1H), 4.40-4.15 (m, 3H), 4.14-4.05 (m, 1H), 1.80 (br s, 3H).

### Example 177

### 1-[2-(3-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one

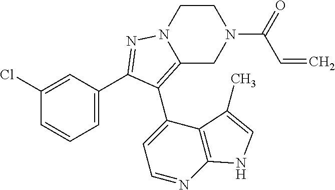 [SMILES: C=CC(=O)N1CCn2nc(-c3cccc(Cl)c3)c(-c3ccnc4[nH]cc(C)c34)c2C1]

To a solution of 2-(3-chlorophenyl)-3-(3-methyl-1H-pyrrolo[2,3-b]pyridin-4-yl)-4,5,6,7-tetrahydropyrazolo[1,5-a]pyrazine trifluoroacetate (1/1) (60.0 mg, 0.126 mmol, Intermediate 263) and prop-2-enoyl chloride (13.6 mg, 0.151 mmol) in dichloromethane (2.0 ml) was added trimethylamine (0.035 ml, 0.251 mmol) at −20° C. The mixture was stirred at −20° C. for 1 hour. The mixture was concentrated in vacuo to give a residue. The residue was purified by preparative-HPLC (column: Phenomenex luna C18 150*25 mm*10 μm; mobile phase: water (TFA)-ACN; B %: 26%-56%, 10 min) to give 1-[2-(3-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one (3.40 mg, 0.00779 mmol, 96% purity, 6% yield) as a white solid.

LC-MS (Method 16): Rₜ=0.819 min; MS (ESIpos): m/z=418.2 [M+H]⁺.

¹H NMR (400 MHz, DMSO-d6), δ [ppm]=11.59-11.41 (m, 1H), 8.24 (d, J=4.8 Hz, 1H), 7.45-7.33 (m, 1H), 7.32-7.15 (m, 4H), 7.06-6.77 (m, 2H), 6.27-6.03 (m, 1H), 5.77 (d, J=10.4 Hz, 1H), 4.61-4.43 (m, 2H), 4.41-4.25 (m, 2H), 4.24-4.04 (m, 2H), 1.71 (s, 3H).

### Example 178

### 1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) prop-2-en-1-one

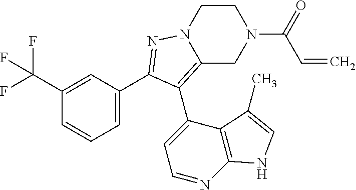 [SMILES: C=CC(=O)N1CCn2nc(-c3cccc(C(F)(F)F)c3)c(-c3ccnc4[nH]cc(C)c34)c2C1]

To a solution of 3-chloro-1-[3-(3-methyl-1H-pyrrolo[2,3-b]pyridin-4-yl)-2-[3-(trifluoromethyl)phenyl]-6,7-dihydropyrazolo[1,5-a]pyrazin-5(4H)-yl]propan-1-one (60.0 mg, 0.123 mmol, Intermediate 267) in acetonitrile (12 ml) was added trimethylamine (0.051 ml, 0.369 mmol) at 20° C. The mixture was stirred at 80° C. for 2 hours. The reaction mixture was concentrated in vacuo to give a residue. The residue was purified by preparative-HPLC (column: Phenomenex luna C18 150*25 mm*10 μm; mobile phase: water (TFA)-ACN;

B %: 31%-61%, 10 min) to give 1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) prop-2-en-1-one (33.5 mg, 0.0723 mmol, 97% purity, 59% yield) as a white solid.

LC-MS (Method 16): Rₜ=0.839 min; MS (ESIpos): m/z=452.2 [M+H]⁺

¹H NMR (400 MHz, DMSO-d6), δ [ppm]=11.48 (br s, 1H), 8.24 (d, J=4.8 Hz, 1H), 7.72-7.61 (m, 1H), 7.59-7.49 (m, 2H), 7.46-7.36 (m, 1H), 7.19 (br s, 1H), 7.06-6.88 (m, 2H), 6.25-6.09 (m, 1H), 5.84-5.65 (m, 1H), 4.63-4.46 (m, 2H), 4.44-4.28 (m, 2H), 4.27-4.06 (m, 2H), 2.07 (s, 1H), 1.69 (s, 3H).

### Example 179

### 1-[2-(2-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one

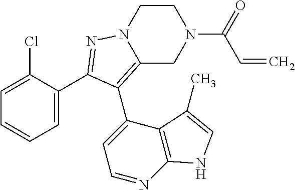 [SMILES: C=CC(=O)N1CCn2nc(-c3ccccc3Cl)c(-c3ccnc4[nH]cc(C)c34)c2C1]

To a solution of 1-[2-(2-chlorophenyl)-3-(3-methyl-1-{[2-(trimethylsilyl) ethoxy]methyl}-1H-pyrrolo[2,3-b]pyridin-4-yl)-6,7-dihydropyrazolo[1,5-a]pyrazin-5(4H)-yl]prop-2-en-1-one (180 mg, 0.328 mmol, Intermediate 270) in tetrahydrofuran (3.4 ml) was added tetra-n-butylammonium fluoride (1M in tetrahydrofuran, 3.0 ml) at 20° C. The mixture was stirred at 20° C. for 3 hours. The reaction mixture was concentrated in vacuo to give a white solid, then the white solid was poured into water and extracted with ethyl acetate. The combined organic layers were washed with brine, dried over anhydrous sodium sulfate, filtered and the filtrate was concentrated in vacuo to give a residue. The residue was dissolved in methanol and then potassium carbonate (90.3 mg, 0.656 mmol) was added. The resulting mixture was stirred at 20° C. for 2 hours. The mixture was concentrated in vacuo to give a residue. The residue was purified by preparative-HPLC (column: Phenomenex luna C18 150*25 mm*10 μm; mobile phase: water (TFA)-ACN; B %: 22%-46%, 8 min) to give 1-[2-(2-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one (18.4 mg, 0.0414 mmol, 94% purity, 13% yield) as a white solid.

LC-MS (Method 16): R₁=0.769 min; MS (ESIpos): m/z=418.2 [M+H]⁺.

¹H NMR (400 MHz, DMSO-d₆), δ [ppm]=11.54-11.24 (m, 1H), 8.05 (d, J=4.8 Hz, 1H), 7.40-7.15 (m, 5H), 6.96 (dd, J=15.2, 10.8 Hz, 1H), 6.75-6.59 (m, 1H), 6.18 (d, J=17.2 Hz, 1H), 5.83-5.63 (m, 1H), 4.71-4.57 (m, 1H), 4.41-4.06 (m, 5H), 1.93 (br s, 3H).

### Example 180

### 1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one

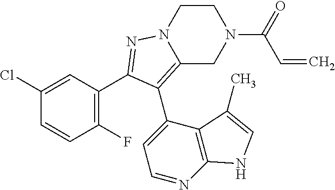 [SMILES: C=CC(=O)N1CCn2nc(-c3cc(Cl)ccc3F)c(-c3ccnc4[nH]cc(C)c34)c2C1]

To a solution of 3-chloro-1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]propan-1-one (90.0 mg, 0.191 mmol, Intermediate 274) in acetonitrile (8.8 ml) was added trimethylamine (0.11 ml, 0.762 mmol) at 20° C. The mixture was stirred at 80° C. for 2 hours. The reaction mixture was concentrated in vacuo to give a residue. The residue was purified by preparative-HPLC (column: Waters Xbridge 150*25 mm*5 μm; mobile phase: water (ammonia hydroxide v/v)-ACN; B %: 32%-62%, 9 min) to give 1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one (20.0 mg, 0.0445 mmol, 97% purity, 23% yield) as a white solid.

LC-MS (Method 20): Rₜ=0.877 min; MS (ESIpos): m/z=436.2 [M+H]⁺.

¹H NMR (400 MHz, DMSO-d₆), δ [ppm]=11.40 (br s, 1H), 8.13 (d, J=4.8 Hz, 1H), 7.35 (dd, J=7.2, 3.6 Hz, 2H), 7.21-7.08 (m, 2H), 6.95 (dd, J=16.4, 10.8 Hz, 1H), 6.82-6.60 (m, 1H), 6.23-6.07 (m, 1H), 5.84-5.62 (m, 1H), 4.79-4.50 (m, 2H), 4.50-4.32 (m, 2H), 4.26-4.05 (m, 2H), 1.80 (br s, 3H).

### Example 181

### 1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]prop-2-en-1-one

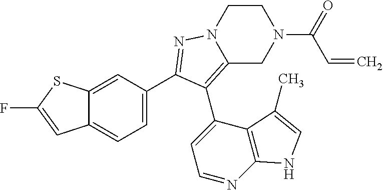 [SMILES: C=CC(=O)N1CCn2nc(-c3ccc4cc(F)sc4c3)c(-c3ccnc4[nH]cc(C)c34)c2C1]

To a solution of 3-chloro-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]propan-1-one (30.0 mg, 0.0607 mmol, Intermediate 280) in acetonitrile (3.0 ml) was added trimethylamine (0.017 ml, 0.121 mmol) at 20° C. The mixture was stirred at 80° C. for 2 hours. The reaction mixture was concentrated in vacuo to give a residue. The residue was purified by preparative-HPLC (column: Phenomenex luna C18 150*25 mm*10 μm; mobile phase: water (TFA)-ACN; B %: 29%-59%, 10 min) to give 1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]prop-2-en-1-one (17.2 mg, 0.0372 mmol, 99% purity, 61% yield) as a white solid.

LC-MS (Method 16): Rₜ=0.838 min; MS (ESIpos): m/z=458.2 [M+H]⁺

¹H NMR (400 MHz, DMSO-d6), δ [ppm]=11.54-11.29 (m, 1H), 8.31-8.17 (m, 1H), 7.91 (br s, 1H), 7.48 (d, J=8.4 Hz, 1H), 7.29-7.11 (m, 2H), 7.03-6.83 (m, 3H), 6.26-6.05 (m, 1H), 5.81-5.62 (m, 1H), 4.50 (br s, 2H), 4.39-4.26 (m, 2H), 4.25-4.06 (m, 2H), 1.71 (br s, 3H).

### Example 182

### 4-[3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile

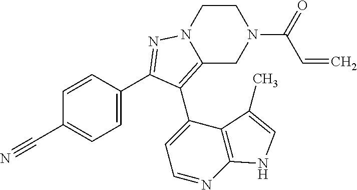 [SMILES: C=CC(=O)N1CCn2nc(-c3ccc(C#N)cc3)c(-c3ccnc4[nH]cc(C)c34)c2C1]

To a solution of 4-[5-(3-chloropropanoyl)-3-(3-methyl-1H-pyrrolo[2,3-b]pyridin-4-yl)-4,5,6,7-tetrahydropyrazolo[1,5-a]pyrazin-2-yl]benzonitrile (20.0 mg, 0.0450 mmol, Intermediate 283) in acetonitrile (10 ml) was added trimethylamine (0.031 ml, 0.225 mmol) at 20° C. The mixture was stirred at 80° C. for 2 hours. The reaction mixture was concentrated in vacuo to give a residue. The residue was purified by preparative HPLC (column: Waters Xbridge 150*25 mm*5 μm; mobile phase: water (ammonia hydroxide v/v)-ACN; B %: 26%-56%, 10 min) to give 4-[3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile (3.30 mg, 0.0100 mmol, 93% purity, 17% yield) as a white solid.

LC-MS (Method 20): Rₜ=0.867 min; MS (ESIpos): m/z=409.1 [M+H]⁺.

¹H NMR (400 MHz, DMSO-d₆), δ [ppm]=11.62-11.40 (m, 1H), 8.24 (d, J=4.8 Hz, 1H), 7.66 (d, J=8.4 Hz, 2H), 7.49 (d, J=8.0 Hz, 2H), 7.19 (br s, 1H), 7.03-6.84 (m, 2H), 6.16 (d, J=16.8 Hz, 1H), 5.84-5.70 (m, 1H), 4.49 (br s, 2H), 4.41-4.28 (m, 2H), 4.21-4.06 (m, 2H), 1.69 (br s, 3H).

### Example 183

### (2E)-1-[2-(4-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one

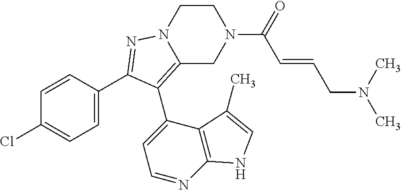 [SMILES: Cc1c[nH]c2nccc(-c3c(-c4ccc(Cl)cc4)nn4c3CN(C(=O)/C=C/CN(C)C)CC4)c12]

To a solution of 2-(4-chlorophenyl)-3-(3-methyl-1H-pyrrolo[2,3-b]pyridin-4-yl)-4,5,6,7-tetrahydropyrazolo[1,5-a]pyrazine trifluoroacetate (1/1) (80.0 mg, 0.167 mmol, Intermediate 257) and (2E)-4-(dimethylamino)but-2-enoic acid hydrogen chloride (1/1) (111 mg, 0.670 mmol) in N,N-dimethylformamide (9.0 ml) was added 1-[bis(dimethylamino)methylene]-1H-1,2,3-triazolo[4,5-b]pyridinium-3-oxide hexafluorophosphate (255 mg, 0.670 mmol) and N,N-diisopropylethylamine (0.15 ml, 0.840 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 250*50 mm*15 μm; mobile phase: water (FA)-ACN; B %: 10%-40%, 10 min) to give (2E)-1-[2-(4-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one (11.5 mg, 0.0219 mmol, 91% purity, 13% yield) as a white solid.

LC-MS (Method 16): Rₜ=0.769 min; MS (ESIpos): m/z=475.3 [M+H]⁺.

¹H NMR (400 MHz, DMSO-d₆), δ [ppm]=11.47 (br s, 1H), 8.25-8.14 (m, 1H), 7.40-7.13 (m, 5H), 6.87 (d, J=3.6 Hz, 1H), 6.79 (d, J=16.0 Hz, 1H), 6.70-6.54 (m, 1H), 4.47 (br s, 2H), 4.38-4.24 (m, 2H), 4.18-4.04 (m, 2H), 3.18-3.04 (m, 2H), 2.30-2.09 (m, 6H), 1.70 (br s, 3H).

### Example 184

### (2E)-1-[2-(3-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one

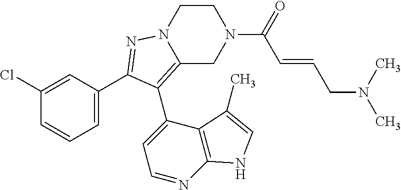 [SMILES: Cc1c[nH]c2nccc(-c3c(-c4cccc(Cl)c4)nn4c3CN(C(=O)/C=C/CN(C)C)CC4)c12]

To a solution of 2-(3-chlorophenyl)-3-(3-methyl-1H-pyrrolo[2,3-b]pyridin-4-yl)-4,5,6,7-tetrahydropyrazolo[1,5-a]pyrazine trifluoroacetate (1/1) (70.0 mg, 0.146 mmol, Intermediate 263) and (2E)-4-(dimethylamino)but-2-enoic acid hydrogen chloride (1/1) (48.5 mg, 0.293 mmol) in N,N-dimethylformamide (8.0 ml) was added 1-[bis(dimethylamino)methylene]-1H-1,2,3-triazolo[4,5-b]pyridinium-3-oxide hexafluorophosphate (111 mg, 0.293 mmol) and N,N-diisopropylethylamine (0.051 ml, 0.293 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Waters Xbridge 150*25 mm*5 μm; mobile phase: water (ammonia hydroxide v/v)-ACN; B %: 25%-55%, 9 min) to give (2E)-1-[2-(3-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one (7.30 mg, 0.0137 mmol, 90% purity, 9% yield) as a white solid.

LC-MS (Method 20): Rₜ=0.904 min; MS (ESIpos): m/z=520.4 [M+H]⁺.

¹H NMR (400 MHz, DMSO-d₆), δ [ppm]=11.49 (br s, 1H), 8.24 (d, J=4.8 Hz, 1H), 7.44-7.34 (m, 1H), 7.28-7.14 (m, 4H), 7.02-6.82 (m, 2H), 6.73-6.58 (m, 1H), 4.57-4.43 (m, 2H), 4.41-4.25 (m, 2H), 4.24-4.06 (m, 2H), 3.52 (br s, 2H), 2.50 (br s, 6H), 1.71 (br s, 3H).

### Example 185

### (E)-4-(dimethylamino)-1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) but-2-en-1-one

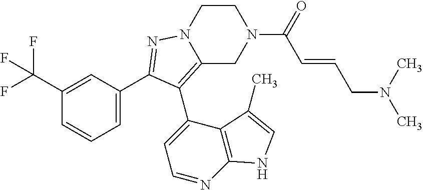 [SMILES: Cc1c[nH]c2nccc(-c3c(-c4cccc(C(F)(F)F)c4)nn4c3CN(C(=O)/C=C/CN(C)C)CC4)c12]

To a solution of 3-(3-methyl-1H-pyrrolo[2,3-b]pyridin-4-yl)-2-[3-(trifluoromethyl)phenyl]-4,5,6,7-tetrahydropyrazolo[1,5-a]pyrazine (60.0 mg, 0.151 mmol, Intermediate 266) and (2E)-4-(dimethylamino)but-2-enoic acid hydrochloride (1:1) (37.5 mg, 0.226 mmol) in N,N-dimethylformamide (3.0 ml) were added 1-[bis(dimethylamino)methylene]-1H-1,2,3-triazolo[4,5-b]pyridinium-3-oxide hexafluorophosphate (86.1 mg, 0.226 mmol) and N,N-diisopropylethylamine (0.053 ml, 0.302 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 150*25 mm*10 μm; mobile phase: water (TFA)-ACN; B %: 11%-41%, 10 min) to give (E)-4-(dimethylamino)-1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) but-2-en-1-one (24.5 mg, 0.0478 mmol, 99% purity, 32% yield) as a white solid.

LC-MS (Method 16): Rₜ=0.787 min; MS (ESIpos): m/z=509.3 [M+H]⁺.

¹H NMR (400 MHz, DMSO-d₆), δ [ppm]=11.57-11.41 (m, 1H), 8.24 (d, J=4.8 Hz, 1H), 7.73-7.60 (m, 1H), 7.57-7.50 (m, 2H), 7.46-7.37 (m, 1H), 7.23-7.16 (m, 1H), 6.96-6.87 (m, 1H), 6.78-6.59 (m, 1H), 4.61-4.42 (m, 2H), 4.41-4.27 (m, 2H), 4.23-4.03 (m, 2H), 3.08-2.95 (m, 2H), 2.19-2.04 (m, 6H), 1.69 (s, 3H).

### Example 186

### (2E)-1-[2-(1-benzofuran-7-yl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one formic acid (1:1)

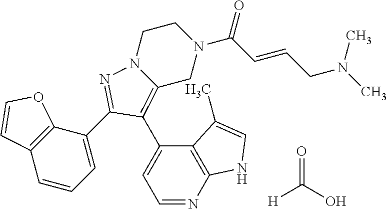

To a solution of 2-(1-benzofuran-7-yl)-3-(3-methyl-1H-pyrrolo[2,3-b]pyridin-4-yl)-4,5,6,7-tetrahydropyrazolo[1,5-a]pyrazine (70.0 mg, 0.189 mmol, Intermediate 286) and (2E)-4-(dimethylamino)but-2-enoic acid (36.7 mg, 0.284 mmol) in N,N-dimethylformamide (2 ml) were added N,N-diisopropylethylamine (73.5 mg, 0.568 mmol) and 1-[bis(dimethylamino)methylene]-1H-1,2,3-triazolo[4,5-b]pyridinium-3-oxide hexafluorophosphate (108 mg, 0.284 mmol) at 20° C. The mixture was stirred at 20° C. for 12 hours. The reaction mixture was poured into water and extracted with ethyl acetate. The combined organic layers were washed with brine, dried over anhydrous sodium sulfate, filtered and concentrated in vacuo to give a residue. The residue was purified by preparative HPLC [Instrument: Gilson-281; Column: Phenomenex luna C18 150*25 mm*10 μm; eluent A: water (0.225% formic acid), eluent B: acetonitrile; gradient: 0-10 min 8-48% B; flow 25 ml/min; temperature: RT; Detector: UV 220/254 nm] to give (2E)-1-[2-(1-benzofuran-7-yl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one formic acid (1:1) (15.6 mg, 0.0283 mmol, 96% purity, 15% yield) as a white solid.

LC-MS (Method 16): Rt=0.420 min; MS (ESIpos): m/z=481.3 [M+H]⁺.

¹H NMR (400 MHz, DMSO-d6), δ [ppm]=11.36 (br s, 1H), 8.15 (br s, 1H), 8.13 (s, 1H), 7.86 (d, J=2.0 Hz, 1H), 7.49 (d, J=7.6 Hz, 1H), 7.12 (br s, 1H), 7.05-6.80 (m, 5H), 6.72-6.59 (m, 1H), 4.84-4.52 (m, 2H), 4.47-4.30 (m, 2H), 4.27-4.06 (m, 2H), 3.81-3.55 (m, 2H), 2.70-2.55 (m, 6H), 1.75 (br s, 3H).

### Example 187

### (2E)-4-(dimethylamino)-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]but-2-en-1-one

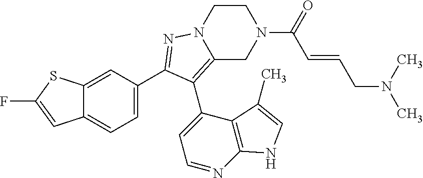 [SMILES: Cc1c[nH]c2nccc(-c3c(-c4ccc5cc(F)sc5c4)nn4c3CN(C(=O)/C=C/CN(C)C)CC4)c12]

To a solution of 2-(2-fluoro-1-benzothiophen-6-yl)-3-(3-methyl-1H-pyrrolo[2,3-b]pyridin-4-yl)-4,5,6,7-tetrahydropyrazolo[1,5-a]pyrazine (30.0 mg, 0.0743 mmol, Intermediate 279) and (2E)-4-(dimethylamino)but-2-enoic acid hydrochloride (1:1) (18.5 mg, 0.112 mmol) in N,N-dimethylformamide (1.8 ml) were added 1-[bis(dimethylamino)methylene]-1H-1,2,3-triazolo[4,5-b]pyridinium-3-oxide hexafluorophosphate (42.4 mg, 0.112 mmol) and N,N-diisopropylethylamine (0.026 ml, 0.149 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C18 150*25 mm*10 μm; mobile phase: water (TFA)-ACN; B %: 12%-42%, 10 min) to give (2E)-4-(dimethylamino)-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]but-2-en-1-one (28.0 mg, 0.0535 mmol, 98% purity, 72% yield) as a white solid.

LC-MS (Method 16): Rₜ=0.771 min; MS (ESIpos): m/z=515.3 [M+H]⁺.

¹H NMR (400 MHz, DMSO-d6), δ [ppm]=11.45 (br s, 1H), 8.23 (d, J=4.8 Hz, 1H), 7.94-7.86 (m, 1H), 7.48 (d, J=8.4 Hz, 1H), 7.24 (d, J=8.8 Hz, 1H), 7.16 (br s, 1H), 6.99 (d, J=2.8 Hz, 1H), 6.89 (d, J=3.6 Hz, 1H), 6.82-6.71 (m, 1H), 6.70-6.53 (m, 1H), 4.73-4.43 (m, 2H), 4.40-4.24 (m, 2H), 4.22-3.98 (m, 2H), 3.09-2.91 (m, 2H), 2.21-2.01 (m, 6H), 1.71 (br s, 3H)

### Example 188

### 1-[3-(pyridin-4-yl)-2-[6-(trifluoromethyl)pyridin-3-yl]-6,7-dihydropyrazolo[1,5-a]pyrazin-5(4H)-yl]prop-2-en-1-one

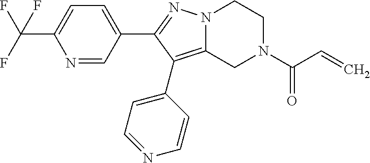 [SMILES: C=CC(=O)N1CCn2nc(-c3ccc(C(F)(F)F)nc3)c(-c3ccncc3)c2C1]

3-(pyridin-4-yl)-2-[6-(trifluoromethyl)pyridin-3-yl]-4,5,6,7-tetrahydropyrazolo[1,5-a]pyrazin-5-ium chloride (96.0 mg, 251 μmol, Intermediate 289) was dissolved in DMF (900 μl), following which prop-2-enoic acid (19.9 mg, 277 μmol), N,N-diisopropylethylamine (260 μl, 1.5 mmol; CAS-RN: [7087-68-5]) and T3P (110 μl, 188 μmol, 50% in DMF, CAS-RN: [68957-94-8]) were added and the mixture stirred for 3.5 h. The mixture was diluted with water and extracted into ethyl acetate (3×), dried over sodium sulfate and concentrated under reduced pressure. Purification by silica gel column chromatography, (Biotage Ultra, Silica 5 g column), 50%-100% ethyl acetate and Hexan yielded the title compound (2.00 mg, 80% purity, 2% yield)

LC-MS (Method 3): Rₜ=0.74 min; MS (ESIpos): m/z=400 [M+H]⁺

¹H-NMR (400 MHz, METHANOL-d4) delta [ppm]: 1.222 (4.60), 1.240 (9.55), 1.258 (4.92), 1.273 (1.22), 1.289 (2.55), 1.472 (1.79), 1.535 (3.36), 2.013 (16.00), 2.862 (0.83), 2.864 (0.88), 2.988 (1.01), 2.996 (1.04), 4.073 (1.27), 4.091 (3.64), 4.108 (3.46), 4.127 (1.20), 4.163 (1.06), 4.177 (1.22), 4.190 (0.78), 4.227 (2.29), 4.240 (4.26), 4.254 (3.28), 4.312 (0.70), 4.318 (0.67), 4.326 (0.80), 4.353 (0.89), 4.369 (1.49), 4.402 (2.36), 4.471 (1.22), 4.487 (0.63), 4.616 (0.96), 4.758 (0.68), 4.956 (2.86), 5.014 (0.73), 5.495 (0.91), 5.821 (0.81), 5.847 (1.04), 5.866 (0.93), 6.287 (0.76), 6.313 (0.78), 6.327 (0.84), 6.351 (0.67), 7.306 (1.28), 7.319 (7.73), 7.324 (5.15), 7.331 (4.81), 7.335 (7.31), 7.794 (4.58), 7.814 (5.43), 8.015 (2.79), 8.020 (2.81), 8.036 (2.29), 8.040 (2.31), 8.109 (0.65), 8.555 (4.91), 8.569 (4.29), 8.589 (0.67), 8.757 (2.81).

### Example 189

### 1-[2-phenyl-3-(pyridin-4-yl)-6,7-dihydropyrazolo[1,5-a]pyrazin-5(4H)-yl]prop-2-en-1-one

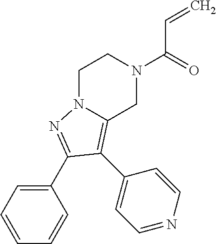 [SMILES: C=CC(=O)N1CCn2nc(-c3ccccc3)c(-c3ccncc3)c2C1]

Analogously to Example 188, the title compound 1-[2-phenyl-3-(pyridin-4-yl)-6,7-dihydropyrazolo[1,5-a]pyrazin-5(4H)-yl]prop-2-en-1-one was prepared from 2-phenyl-3-(pyridin-4-yl)-4,5,6,7-tetrahydropyrazolo[1,5-a]pyrazin-5-ium chloride (Intermediate 290) and prop-2-enoic acid.

LC-MS (Method 3): Rₜ=0.68 min; MS (ESIpos): m/z=331 [M+H]⁺

¹H-NMR (400 MHz, METHANOL-d4) delta [ppm]: 0.881 (0.53), 0.899 (0.97), 0.906 (0.57), 0.916 (0.59), 0.924 (0.84), 0.955 (0.65), 0.974 (1.31), 0.992 (0.63), 1.178 (0.69), 1.221 (1.57), 1.239 (3.19), 1.256 (1.74), 1.260 (0.79), 1.273 (0.85), 1.286 (4.90), 1.305 (1.13), 1.338 (0.66), 1.354 (0.52), 1.366 (0.53), 1.529 (0.56), 2.012 (4.75), 2.270 (1.13), 2.860 (0.53), 2.876 (0.81), 2.892 (0.49), 2.919 (0.56), 2.937 (0.50), 2.962 (0.74), 2.978 (1.40), 2.993 (0.85), 4.071 (0.57), 4.089 (1.24), 4.107 (1.24), 4.122 (0.79), 4.124 (0.79), 4.131 (0.87), 4.143 (1.38), 4.157 (1.24), 4.180 (0.71), 4.209 (2.24), 4.221 (4.56), 4.236 (3.81), 4.251 (0.85). 4.265 (1.03), 4.278 (1.05), 4.282 (1.00), 4.293 (1.02), 4.295 (1.12), 4.340 (2.59), 4.352 (2.55), 4.367 (1.41), 4.403 (0.69), 4.418 (1.16), 4.434 (0.60), 4.450 (0.81), 4.466 (1.56), 4.482 (0.74), 4.846 (0.69), 4.952 (2.80), 5.009 (0.77), 5.493 (1.24), 5.808 (0.75), 5.838 (0.87), 5.858 (1.05), 5.862 (1.13), 5.884 (0.71), 5.888 (0.71), 6.108 (0.50), 6.152 (0.65), 6.178 (0.54), 6.256 (0.56), 6.283 (0.74), 6.295 (0.71), 6.306 (0.54), 6.310 (0.57), 6.325 (0.77), 6.347 (0.81), 6.351 (0.81), 6.390 (0.50), 6.394 (0.49), 7.244 (4.06), 7.255 (3.74), 7.314 (1.00), 7.326 (2.47), 7.335 (4.86), 7.340 (5.90), 7.348 (11.13), 7.354 (16.00), 7.359 (6.59), 7.362 (5.95), 7.369 (6.74), 7.378 (4.24), 7.388 (2.49), 7.393 (1.62), 8.104 (2.40), 8.473 (1.53).

### Example 190

### 1-[2-(4-chloro-3-ethylphenyl)-3-(pyridin-4-yl)-6,7-dihydropyrazolo[1,5-a]pyrazin-5(4H)-yl]prop-2-en-1-one

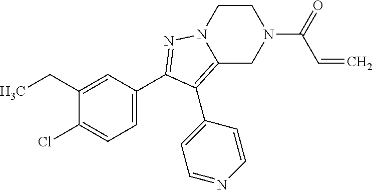 [SMILES: C=CC(=O)N1CCn2nc(-c3ccc(Cl)c(CC)c3)c(-c3ccncc3)c2C1]

To a slurry of 2-(4-chloro-3-ethylphenyl)-3-(pyridin-4-yl)-4,5,6,7-tetrahydropyrazolo[1,5-a]pyrazine (30.0 mg, 55.77 μmol, Intermediate 292) and triethylamine (39 μl) in dichloromethane (5 ml) was added dropwise a solution of acryloyl chloride (5.56 mg, 61.4 μmol) in dichloromethane (0.5 mL) at 25° C. The mixture was stirred at 25° C. for 1 hour. The reaction mixture was poured into water and extracted with ethyl acetate. The organic phase was washed with brine, dried over anhydrous sodium sulfate, filtered and concentrated in vacuo. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 38-68% B; flow 25 ml/min; temperature: room temperature; Detector: UV 220/254 nm) to give 1-[2-(4-chloro-3-ethylphenyl)-3-(pyridin-4-yl)-6,7-dihydropyrazolo[1,5-a]pyrazin-5(4H)-yl]prop-2-en-1-one (8.8 mg, 98% purity, 39% yield) as a white solid.

LC-MS (Method 20): Rₜ=0.951 min; MS (ESIpos): m/z=393.0 [M+H]⁺.

¹H NMR (400 MHz, DMSO-d6) δ [ppm]=8.56 (d. J=4.0 Hz, 2H), 7.38 (d, J=4.0 Hz, 2H), 7.31 (s, 1H), 7.25-7.13 (m, 3H), 7.05-6.32 (m, 1H), 6.17 (d, J=16.0 Hz, 1H), 5.85-5.15 (m, 1H), 5.05-4.75 (m, 2H), 4.35-4.01 (m, 4H), 2.61 (q, J=8.0 Hz, 2H), 1.06 (t, J=8.0 Hz, 3H).

### Example 191

### 1-{2-[4-chloro-2-(hydroxymethyl)phenyl]-3-(pyridin-4-yl)-6,7-dihydropyrazolo[1,5-a]pyrazin-5(4H)-yl}prop-2-en-1-one

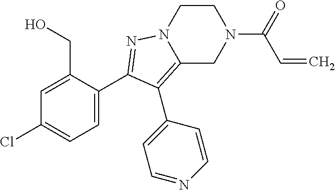 [SMILES: C=CC(=O)N1CCn2nc(-c3ccc(Cl)cc3CO)c(-c3ccncc3)c2C1]

To a slurry of {5-chloro-2-[3-(pyridin-4-yl)-4,5,6,7-tetrahydropyrazolo[1,5-a]pyrazin-2-yl]phenyl}methanol (90.0 mg, 235 μmol, Intermediate 294) in dichloromethane (5 ml) were added trimethylamine (164 μl) and a solution of acryloyl chloride (23.4 mg, 258 μmol) in dichloromethane (1 ml) at 25° C. The mixture was stirred at 25° C. for 1 hour. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 20-50% B; flow 25 ml/min; temperature: room temperature; Detector: UV 220/254 nm) to give 1-{2-[4-chloro-2-(hydroxymethyl)phenyl]-3-(pyridin-4-yl)-6,7-dihydropyrazolo[1,5-a]pyrazin-5(4H)-yl}prop-2-en-1-one (35 mg, 99% purity, 37% yield) as a white solid.

LC-MS (Method 20): Rₜ=0.824 min; MS (ESIpos): m/z=395.0 [M+H]⁺.

¹H NMR (400 MHz, DMSO-d₆) δ [ppm]=8.47 (s, 2H), 7.57 (d, J=2.4 Hz, 1H), 7.30 (dd, J=2.4, 2.0 Hz, 1H), 7.09 (d, J=8.4 Hz, 1H), 7.06-6.88 (m, 3H), 6.19 (dd, J=2.0, 2.0 Hz, 1H), 5.88-5.68 (m, 1H), 5.22 (t, J=5.6 Hz, 2H), 5.12-4.88 (m, 2H), 4.40-4.05 (m, 6H).

### Example 192

### 1-[2-(1-benzothiophen-5-yl)-3-(pyridin-4-yl)-6,7-dihydropyrazolo[1,5-a]pyrazin-5(4H)-yl]prop-2-en-1-one

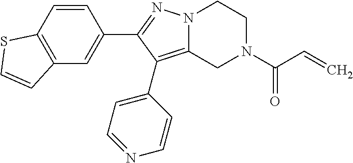 [SMILES: C=CC(=O)N1CCn2nc(-c3ccc4sccc4c3)c(-c3ccncc3)c2C1]

To a solution of 2-(1-benzothiophen-5-yl)-3-(pyridin-4-yl)-4,5,6,7-tetrahydropyrazolo[1,5-a]pyrazine hydrogen chloride (100 mg, 271 μmol, Intermediate 296) and trimethylamine (110 μl, 810 μmol) in dichloromethane (5 ml) was added acryloyl chloride (24.5 mg, 271 μmol) at 25° C. and the reaction mixture was stirred at 25° C. for 1 hour. The reaction mixture was concentrated under vacuum to give a crude product and the crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 27-57% B; flow 25 ml/min; temperature: RT; Detector: UV 220/254 nm] to give 1-[2-(1-benzothiophen-5-yl)-3-(pyridin-4-yl)-6,7-dihydropyrazolo[1,5-a]pyrazin-5(4H)-yl]prop-2-en-1-one (34.5 mg, 86.6 μmol, 97% purity, 32% yield) as a grey solid.

LC-MS (Method 20): Rₜ=0.885 min; MS (ESIpos): m/z=387.0 [M+H]⁺

¹H NMR (400 MHz, DMSO-d₆) δ [ppm]=8.53 (d, J=3.6 Hz, 2H), 7.97 (d, J=8.4 Hz, 1H), 7.90 (s, 1H), 7.78 (d, J=5.6 Hz, 1H), 7.43 (d, J=5.6 Hz, 1H), 7.29 (d, J=7.6 Hz, 1H), 7.18 (s, 2H), 7.06-6.81 (m, 1H), 6.19 (d, J=16.0 Hz, 1H), 5.89-5.64 (m, 1H), 5.10-4.70 (m, 2H), 4.38-3.99 (m, 4H).

### Example 193

### 1-[2-(1H-indol-5-yl)-3-(pyridin-4-yl)-6,7-dihydropyrazolo[1,5-a]pyrazin-5(4H)-yl]prop-2-en-1-one

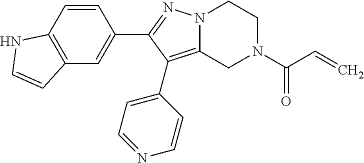 [SMILES: C=CC(=O)N1CCn2nc(-c3ccc4[nH]ccc4c3)c(-c3ccncc3)c2C1]

To a solution of 2-(1H-indol-5-yl)-3-(pyridin-4-yl)-4,5,6,7-tetrahydropyrazolo[1,5-a]pyrazine hydrochloride (1:1) (150 mg, 476 μmol, Intermediate 298) and acryloyl chloride (51.7 mg, 0.571 mmol) in dichloromethane (8.0 mL) were added Triethylamine (86.2 mg, 0.852 mmol) at 20° C. The reaction mixture was stirred at 20° C. for 2 hours. The reaction solution was concentrated in vacuo to give a residue. The residue was purified by pre-HPLC (column: Phenomenex luna C18 150*25 mm*10 μm; mobile phase: water (FA)-ACN; B %: 3%-36%, 12 min) to give 1-[2-(1H-indol-5-yl)-3-(pyridin-4-yl)-6,7-dihydropyrazolo[1,5-a]pyrazin-5(4H)-yl]prop-2-en-1-one (3.10 mg, 0.086 mmol, 1.9% yield) as a white solid.

LC-MS (Method 16): Rₜ=0.862 min; MS (ESIpos): m/z=369.1 [M+H]⁺.

¹H NMR (400 MHz, DMSO-d₆) δ [ppm]=11.26-11.07 (m, 1H), 8.53-8.45 (m, 3H), 7.52 (s, 1H), 7.41-7.31 (m, 3H), 7.15 (br s, 2H), 7.06 (d, J=8.0 Hz, 1H), 7.00-6.86 (m, 1H), 6.38 (d, J=3.2 Hz, 1H), 6.18 (d, J=16.8 Hz, 1H), 5.88-5.65 (m, 1H), 4.99-4.83 (m, 2H), 4.30-4.22 (m, 2H), 4.21-4.09 (m, 2H).

### Example 194

### 1-[2-(1-benzofuran-5-yl)-3-(pyridin-4-yl)-6,7-dihydropyrazolo[1,5-a]pyrazin-5(4H)-yl]prop-2-en-1-one

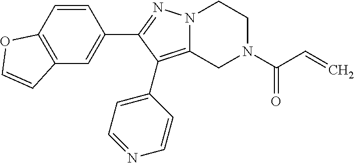 [SMILES: C=CC(=O)N1CCn2nc(-c3ccc4occc4c3)c(-c3ccncc3)c2C1]

To a solution of 2-(1-benzofuran-5-yl)-3-(pyridin-4-yl)-4,5,6,7-tetrahydropyrazolo[1,5-a]pyrazine hydrochloride (1:1) (130 mg, 0.411 mmol, Intermediate 300) and acryloyl chloride (44.6 mg, 0.493 mmol) in dichloromethane (5 ml) was added trimethylamine (74.5 mg, 0.736 mmol) at 20° C. The mixture was stirred at 20° C. for 2 hours. The reaction mixture was concentrated in vacuo to give a residue. The residue was purified by preparative HPLC (column Unisil 3-100 C18 Ultra 150*50 mm*3 μm; mobile phase: water (FA)-ACN; B %: 4%-34%, 7 min) to give 1-[2-(1-benzofuran-5-yl)-3-(pyridin-4-yl)-6,7-dihydropyrazolo[1,5-a]pyrazin-5(4H)-yl]prop-2-en-1-one (6.6 mg, 0.0177 mmol, 99% yield) as a white solid.

LC-MS (Method 16): Rₜ=0.444 min; MS (ESIpos): m/z=370.1 [M+H]⁺.

¹H NMR (400 MHz, DMSO-da) δ [ppm]=8.51 (br s, 2H), 8.00 (d, J=2.0 Hz, 1H), 7.63 (s, 1H), 7.57 (d, J=8.4 Hz, 1H), 7.27 (d, J=8.4 Hz, 1H), 7.16 (br s, 2H), 6.94 (d, J=1.6 Hz, 2H), 6.18 (d, J=16.4 Hz, 1H), 5.79 (d, J=10.4 Hz, 1H), 5.04-4.80 (m, 2H), 4.29 (br s, 2H), 4.21-4.07 (m, 2H).

### Example 195

### 1-[2-(1H-indol-6-yl)-3-(pyridin-4-yl)-6,7-dihydropyrazolo[1,5-a]pyrazin-5(4H)-yl]prop-2-en-1-one

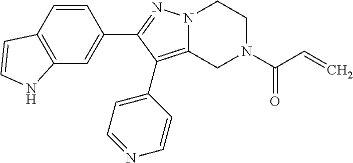 [SMILES: C=CC(=O)N1CCn2nc(-c3ccc4cc[nH]c4c3)c(-c3ccncc3)c2C1]

To a solution of 2-(1H-indol-6-yl)-3-(pyridin-4-yl)-4,5,6,7-tetrahydropyrazolo[1,5-a]pyrazine hydrochloride (1:1) (150 mg, 0.476 mmol, Intermediate 302) and acryloyl chloride (51.7 mg, 0.571 mmol) in dichloromethane (5 ml) was added trimethylamine (86.2 mg, 0.852 mmol) at 20° C. The mixture was stirred at 20° C. for 2 hours. The reaction solution was concentrated in vacuo to give a residue. The residue was purified by preparative HPLC (column: Phenomenex luna C18 150*40 mm*15 μm; mobile phase: water (TFA)-ACN; B %: 3%-33%, 9 min) to give 1-[2-(1H-indol-6-yl)-3-(pyridin-4-yl)-6,7-dihydropyrazolo[1,5-a]pyrazin-5(4H)-yl]prop-2-en-1-one (9.30 mg, 0.0244 mmol, 6% yield) as a white solid.

LC-MS (Method 20): Rₜ=0.881 min; MS (ESIpos): m/z=369.1 [M+H]⁺.

¹H NMR (400 MHz, DMSO-d₆) δ [ppm]=11.09 (br s, 1H), 8.51 (d, J=3.6 Hz, 2H), 7.48 (d, J=8.4 Hz, 1H), 7.39-7.32 (m, 2H), 7.18 (br s, 2H), 7.02 (d, J=8.8 Hz, 1H), 6.98-6.81 (m, 1H), 6.41 (br s, 1H), 6.18 (d, J=16.0 Hz, 1H), 5.92-5.66 (m, 1H), 4.98-4.82 (m, 2H), 4.32-4.26 (m, 2H), 4.18-4.11 (m, 2H).

### Example 196

### (2E)-1-[2-(1-benzothiophen-5-yl)-3-(pyridin-4-yl)-6,7-dihydropyrazolo[1,5-a]pyrazin-5(4H)-yl]-4-(dimethylamino)but-2-en-1-one

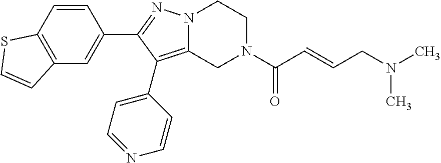 [SMILES: CN(C)C/C=C/C(=O)N1CCn2nc(-c3ccc4sccc4c3)c(-c3ccncc3)c2C1]

To a solution of (2E)-4-(dimethylamino)but-2-enoic acid hydrogen chloride (44.9 mg, 271 μmol) in dichloromethane (5 ml) were added N,N-diisopropylethylamine (190 μl, 1.1 mmol) and 1-[bis(dimethylamino)methylene]-1H-1,2,3-triazolo[4,5-b]pyridinium-3-oxide hexafluorophosphate (155 mg, 407 μmol) at 25° C. The mixture was stirred at 25° C. for 0.1 hour. To this solution was added 2-(1-benzothiophen-5-yl)-3-(pyridin-4-yl)-4,5,6,7-tetrahydropyrazolo[1,5-a]pyrazine hydrogen chloride (100 mg, 271 μmol, Intermediate 296) at 25° C. and the resulting mixture was stirred at 25° C. for 1 hour. The reaction mixture was concentrated under vacuum to give a crude product and the crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 30-60% B; flow 25 ml/min; temperature: RT; Detector: UV 220/254 nm] to give (2E)-1-[2-(1-benzothiophen-5-yl)-3-(pyridin-4-yl)-6,7-dihydropyrazolo[1,5-a]pyrazin-5(4H)-yl]-4-(dimethylamino)but-2-en-1-one (41.8 mg, 89.5 μmol, 95% purity, 33% yield) as a grey solid.

LC-MS (Method 20): Rₜ=0.889 min; MS (ESIpos): m/z=444.1 [M+H]⁺.

¹H NMR (400 MHz, DMSO-da) δ [ppm]=8.53 (d, J=5.6 Hz, 2H), 7.96 (d, J=8.4 Hz, 1H), 7.89 (s, 1H), 7.78 (d, J=5.6 Hz, 1H), 7.43 (d, J=5.6 Hz, 1H), 7.33-7.24 (m, 1H), 7.17 (d, J=4.0 Hz, 2H), 6.83-6.58 (m, 2H), 5.08-4.72 (m, 2H), 4.39-3.97 (m, 4H), 3.05 (s, 2H), 2.16 (s, 6H).

### Example 197

### (2E)-4-(dimethylamino)-1-[2-(1H-indol-5-yl)-3-(pyridin-4-yl)-6,7-dihydropyrazolo[1,5-a]pyrazin-5(4H)-yl]but-2-en-1-one

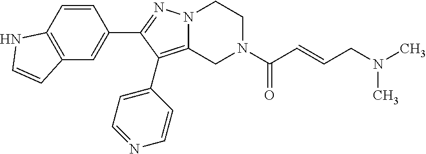 [SMILES: CN(C)C/C=C/C(=O)N1CCn2nc(-c3ccc4[nH]ccc4c3)c(-c3ccncc3)c2C1]

To a solution of 2-(1H-indol-5-yl)-3-(pyridin-4-yl)-4,5,6,7-tetrahydropyrazolo[1,5-a]pyrazine (150 mg, 0.476 mmol, Intermediate 298) and 2E)-4-(dimethylamino)but-2-enoic acid hydrochloride (1:1) (92.1 mg, 0.713 mmol) in N,N-dimethylformamide (8 ml) were added 1-[bis(dimethylamino)methylene]-1H-1,2,3-triazolo[4,5-b]pyridinium-3-oxide hexafluorophosphate ((271 mg, 0.713 mmol) and N,N-diisopropylethylamine (170 μl, 0.950 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 17%-47%, 9 min) to give (2E)-4-(dimethylamino)-1-[2-(1H-indol-5-yl)-3-(pyridin-4-yl)-6,7-dihydropyrazolo[1,5-a]pyrazin-5(4H)-yl]but-2-en-1-one (26.6 mg, 0.0583 mmol, 13% yield) as a white solid.

LC-MS (Method 20): Rₜ=0.495 min; MS (ESIpos): m/z=426.2 [M+H]⁺.

¹H NMR (400 MHz, DMSO-d₆) δ [ppm]=11.15 (br s, 1H), 8.49 (d, J=4.8 Hz, 2H), 7.52 (s, 1H), 7.37-7.32 (m, 2H), 7.15 (br s, 2H), 7.06 (d, J=8.4 Hz, 1H), 6.84-6.60 (m, 2H), 6.38 (br s, 1H), 4.99-4.83 (m, 2H), 4.30-4.23 (m, 2H), 4.15 (d, J=2.8 Hz, 2H), 3.10-2.98 (m, 4H), 2.18-2.09 (m, 6H).

### Example 198

### (2E)-1-[2-(1H-benzotriazol-6-yl)-3-(pyridin-4-yl)-6,7-dihydropyrazolo[1,5-a]pyrazin-5(4H)-yl]-4-(dimethylamino)but-2-en-1-one

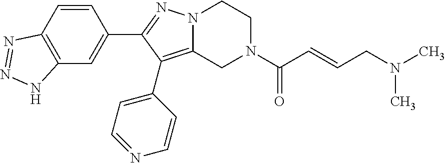 [SMILES: CN(C)C/C=C/C(=O)N1CCn2nc(-c3ccc4nn[nH]c4c3)c(-c3ccncc3)c2C1]

To a solution of (2E)-4-(dimethylamino)but-2-enoic acid hydrochloride (37.4 mg, 226 μmol) in dichloromethane (6 ml) were added 1-[bis(dimethylamino)methylene]-1H-1,2,3-triazolo[4,5-b]pyridinium-3-oxide hexafluorophosphate (129 mg, 339 μmol) and N,N-diisopropylethylamine (117 mg, 904 μmol) at 25° C. and the mixture was stirred at 25° C. for 0.1 hour. To this solution was added 6-[3-(pyridin-4-yl)-4,5,6,7-tetrahydropyrazolo[1,5-a]pyrazin-2-yl]-1H-benzotriazole hydrochloride (80.0 mg, 226 μmol, Intermediate 306) at 25° C. and the resulting mixture was stirred at 25° C. for 2 hours. The reaction mixture was concentrated under vacuum to give a residue. The residue was purified by preparative TLC (ethyl acetate:methanol=3:1) to give a crude product and the crude product was purified by preparative HPLC [Instrument: ACSWH-GX-A; Column: Waters Xbridge 150*25 mm*5 um, eluent A: water (0.2% ammonia hydroxide), eluent B: acetonitrile; gradient: 0-9 min 0-15% B; flow 25 ml/min; temperature: RT; Detector: UV 220/254 nm] to give (2E)-1-[2-(1H-benzotriazol-6-yl)-3-(pyridin-4-yl)-6,7-dihydropyrazolo[1,5-a]pyrazin-5(4H)-yl]-4-(dimethylamino)but-2-en-1-one (5.00 mg, 11.3 μmol, 97% purity, 5% yield) as a white solid.

LC-MS (Method 20): Rₜ=0.649 min; MS (ESIpos): m/z=429.2 [M+H]⁺

¹H NMR (400 MHz, DMSO-d₆) δ [ppm]=8.53 (d, J=5.6 Hz, 2H), 7.84 (d, J=8.8 Hz, 1H), 7.72 (s, 1H), 7.36 (d, J=6.0 Hz, 1H), 7.18 (d, J=4.4 Hz, 2H), 6.83-6.53 (m, 2H), 5.10-4.66 (m, 2H), 4.40-4.07 (m, 4H), 3.06 (d, J=5.2 Hz, 2H), 2.18-2.06 (m, 6H).

### Example 199

### (2E)-1-[2-(1-benzofuran-5-yl)-3-(pyridin-4-yl)-6,7-dihydropyrazolo[1,5-a]pyrazin-5(4H)-yl]-4-(dimethylamino)but-2-en-1-one

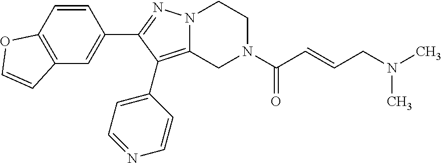 [SMILES: CN(C)C/C=C/C(=O)N1CCn2nc(-c3ccc4occc4c3)c(-c3ccncc3)c2C1]

To a solution of 2-(1-benzofuran-5-yl)-3-(pyridin-4-yl)-4,5,6,7-tetrahydropyrazolo[1,5-a]pyrazine (150 mg, 0.474 mmol, Intermediate 300) and (2E)-4-(dimethylamino)but-2-enoic acid hydrogen chloride (1:1) (118 mg, 0.711 mmol) in N,N-dimethylformamide (2.0 mL) were added 1-[bis(dimethylamino)methylene]-1H-1,2,3-triazolo[4,5-b]pyridinium-3-oxide hexafluorophosphate (270 mg, 0.711 mmol) and N,N-diisopropylethylamine (170 μl, 0.950 mmol) at 20° C. The mixture was stirred at 20° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 24%-54%, 9 min) to give (2E)-1-[2-(1-benzofuran-5-yl)-3-(pyridin-4-yl)-6,7-dihydropyrazolo[1,5-a]pyrazin-5(4H)-yl]-4-(dimethylamino)but-2-en-1-one (23.7 mg, 0.0525 mmol, 95% purity, 12% yield) as a white solid.

LC-MS (Method 16): Rₜ=0.418 min; MS (ESIpos): m/z=427.2 [M+H]⁺.

¹H NMR (400 MHz, DMSO-d6) δ [ppm]=8.52 (d, J=5.2 Hz, 2H), 8.00 (d, J=2.0 Hz, 1H), 7.62 (s, 1H), 7.57 (d, J=8.4 Hz, 1H), 7.27 (d, J=8.0 Hz, 1H), 7.15 (br s, 2H), 7.02-6.78 (m, 2H), 6.74-6.57 (m, 1H), 5.06-4.73 (m, 2H), 4.37-4.22 (m, 2H), 4.21-4.06 (m, 2H), 2.34 (br s, 6H).

### Example 200

### (2E)-4-(dimethylamino)-1-[2-(1H-indol-6-yl)-3-(pyridin-4-yl)-6,7-dihydropyrazolo[1,5-a]pyrazin-5(4H)-yl]but-2-en-1-one

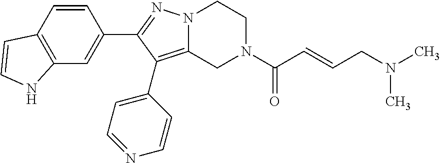 [SMILES: CN(C)C/C=C/C(=O)N1CCn2nc(-c3ccc4cc[nH]c4c3)c(-c3ccncc3)c2C1]

To a solution of 2-(1H-indol-6-yl)-3-(pyridin-4-yl)-4,5,6,7-tetrahydropyrazolo[1,5-a]pyrazine (150 mg, 0.476 mmol, Intermediate 302) and (2E)-4-(dimethylamino)but-2-enoic acid hydrogen chloride (1:1) (118 mg, 0.713 mmol) in N,N-dimethylformamide (8.0 ml) were added 1-[bis(dimethylamino)methylene]-1H-1,2,3-triazolo[4,5-b]pyridinium-3-oxide hexafluorophosphate (271 mg, 0.713 mmol) and N,N-diisopropylethylamine (170 μl, 0.950 mmol) at 20° C. The mixture was stirred at 20° C. for 16 hours. The reaction solution was poured into water and extracted with ethyl acetate. The combined organic layers were washed with brine, dried over anhydrous sodium sulfate, then filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 20%-50%, 9 min) to give (2E)-4-(dimethylamino)-1-[2-(1H-indol-6-yl)-3-(pyridin-4-yl)-6,7-dihydropyrazolo[1,5-a]pyrazin-5(4H)-yl]but-2-en-1-one (26.3 mg, 0.0592 mmol, 96% purity, 13% yield) as a white solid.

LC-MS (Method 20): Rₜ=0.850 min; MS (ESIpos): m/z=426.2 [M+H]⁺

¹H NMR (400 MHz, DMSO-d₆) δ [ppm]=11.09 (br s, 1H), 8.51 (d, J=5.6 Hz, 2H), 7.48 (d, J=8.4 Hz, 1H), 7.39-7.33 (m, 2H), 7.17 (d, J=4.4 Hz, 2H), 7.02 (d, J=7.6 Hz, 1H), 6.81-6.59 (m, 2H), 6.46-6.35 (m, 1H), 5.03-4.75 (m, 2H), 4.34-4.19 (m, 2H), 4.19-4.04 (m, 2H), 3.19-2.90 (m, 3H), 2.20-2.04 (m, 7H).

### Example 201

### rac-(R,E)-4-(dimethylamino)-1-(2-(4-fluorophenyl)-7-(hydroxymethyl)-3-(pyridin-4-yl)-6,7-dihydropyrazolo[1,5-a]pyrazin-5(4H)-yl) but-2-en-1-one

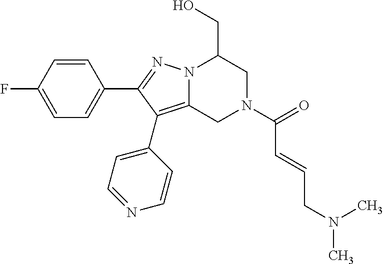 [SMILES: CN(C)C/C=C/C(=O)N1Cc2c(-c3ccncc3)c(-c3ccc(F)cc3)nn2C(CO)C1]

To a solution of [(7RS)-2-(4-fluorophenyl)-3-(pyridin-4-yl)-4,5,6,7-tetrahydropyrazolo[1,5-a]pyrazin-7-yl]methanol hydrochloride (1:1) (200 mg, 0.617 mmol, Intermediate 318) and (2E)-4-(dimethylamino)but-2-enoic acid hydrochloride (1:1) (138 mg, 0.831 mmol) in N,N-dimethylformamide (10 ml) were added 1-[bis(dimethylamino)methylene]-1H-1,2,3-triazolo[4,5-b]pyridinium-3-oxide hexafluorophosphate (316 mg, 0.925 mmol) and N,N-diisopropylethylamine (0.29 ml, 1.66 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Waters Xbridge 150*25 mm*5 μm; mobile phase: water (ammonia hydroxide v/v)-ACN; B %: 22%-52%, 9 min) to give rac-(R,E)-4-(dimethylamino)-1-(2-(4-fluorophenyl)-7-(hydroxymethyl)-3-(pyridin-4-yl)-6,7-dihydropyrazolo[1,5-a]pyrazin-5(4H)-yl) but-2-en-1-one (3.90 mg, 98% purity, 0.00882 mmol, 2% yield) as a white solid.

LC-MS (Method 20): Rₜ=0.841 min; MS (ESIpos): m/z=436.2 [M+H]⁺.

¹H NMR (400 MHz, DMSO-d₆) δ [ppm]=8.55 (d, J=4.4 Hz, 2H), 7.37 (d, J=4.8 Hz, 2H), 7.23-7.12 (m, 4H), 6.85-6.73 (m, 1H), 6.72-6.56 (m, 1H), 6.90-6.53 (m, 1H), 6.92-6.48 (m, 1H), 5.35-5.08 (m, 1H), 4.92 (d, J=16.8 Hz, 1H), 4.73 (d, J=16.8 Hz, 1H), 4.45-4.28 (m, 1H), 4.28-4.17 (m, 1H), 4.14-4.07 (m, 1H), 3.94 (d, J=8.4 Hz, 1H), 3.73 (d, J=7.2 Hz, 2H), 2.99-2.83 (m, 1H), 2.40-2.19 (m, 6H).

### Example 202

### (7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazine-7-carboxamide

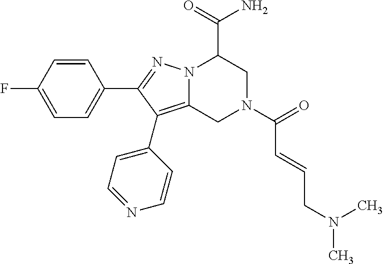 [SMILES: CN(C)C/C=C/C(=O)N1Cc2c(-c3ccncc3)c(-c3ccc(F)cc3)nn2C(C(N)=O)C1]

To a solution of (7RS)-2-(4-fluorophenyl)-3-(pyridin-4-yl)-4,5,6,7-tetrahydropyrazolo[1,5-a]pyrazine-7-carboxamide hydrogen chloride (1/1) (110 mg, 326 μmol, Intermediate 322) and (2E)-4-(dimethylamino)but-2-enoic acid hydrochloride (1:1) (64.8 mg, 391 μmol) in N,N-dimethylformamide were added 1-[bis(dimethylamino)methylene]-1H-1,2,3-triazolo[4,5-b]pyridinium-3-oxide hexafluorophosphate (248 mg, 652 μmol) and N,N-diisopropylethylamine (126 mg, 978 μmol) at 25° C. The mixture was stirred at 25° C. for 16 hours. The reaction mixture was poured into water and extracted with the mixed solvents of dichloromethane and methanol (10:1). The combined organic phase was concentrated in vacuo to give a crude product. The crude product was purified by preparative TLC (ethyl acetate:methanol:NH₃·H2O=10:1:0.1) and preparative HPLC [Instrument: GX-A; Column: Waters Xbridge 150*25 mm*5 μm; eluent A: water (0.2% NH₃·H₂O), eluent B: acetonitrile; gradient: 0-10 min 15-45% B; flow 25 ml/min; temperature: RT; Detector: UV 220/254 nm] to give (7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazine-7-carboxamide (0.500 mg, 1.06 μmol, 0.3% yield) as a white solid.

LC-MS (Method 20): Rₜ=0.796 min; MS (ESIpos): m/z=449.3 [M+H]⁺

1H NMR (400 MHz, DMSO-d6), δ [ppm]=8.56 (d, J=4.8 Hz, 2H), 7.78-7.51 (m, 1H), 7.38 (d, J=6.4 Hz, 2H), 7.19 (q, J=8.8 Hz, 4H), 6.63 (s, 1H), 5.17-4.92 (m, 2H), 4.87-4.65 (m, 1H), 4.60-4.40 (m, 1H), 4.22-3.74 (m, 1H), 3.12-3.01 (m, 2H), 2.20-2.03 (m, 6H).

### Example 203

### 1-[(7RS)-2-(4-fluorophenyl)-7-(hydroxymethyl)-3-(pyridin-4-yl)-6,7-dihydropyrazolo[1,5-a]pyrazin-5(4H)-yl]prop-2-en-1-one

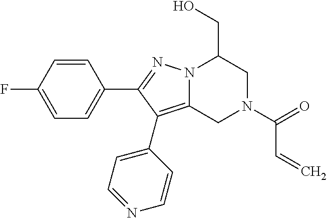 [SMILES: C=CC(=O)N1Cc2c(-c3ccncc3)c(-c3ccc(F)cc3)nn2C(CO)C1]

To a solution of [(7RS)-2-(4-fluorophenyl)-3-(pyridin-4-yl)-4,5,6,7-tetrahydropyrazolo[1,5-a]pyrazin-7-yl]methanol hydrochloride (1:1) (200 mg, 0.554 mmol, Intermediate 318) and prop-2-enoic acid (47.9 mg, 0.665 mmol) in N,N-dimethylformamide (6 ml) were added 1-[bis(dimethylamino)methylene]-1H-1,2,3-triazolo[4,5-b]pyridinium-3-oxide hexafluorophosphate (316 mg, 0.831 mmol) and N,N-diisopropylethylamine (0.3 ml, 1.66 mmol) at 20° C. The mixture was stirred at 20° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Waters Xbridge 150*25 mm*5 μm; mobile phase: water (NH4HCO3)-ACN;

B %: 23%-53%, 8 min) to give 1-[(7RS)-2-(4-fluorophenyl)-7-(hydroxymethyl)-3-(pyridin-4-yl)-6,7-dihydropyrazolo[1,5-a]pyrazin-5(4H)-yl]prop-2-en-1-one (4.10 mg, 98% purity, 0.0107 mmol, 2% yield) as a white solid.

LC-MS (Method 20): Rₜ=0.824 min; MS (ESIpos): m/z=379.1 [M+H]⁺.

¹H NMR (400 MHz, DMSO-d6), δ [ppm]=8.55 (d, J=4.8 Hz, 2H), 7.51-7.32 (m, 2H), 7.30-7.11 (m, 4H), 7.03-6.73 (m, 1H), 6.31-6.00 (m, 1H), 5.88-5.59 (m, 1H), 5.37-5.06 (m, 1H), 5.04-4.81 (m, 1H), 4.80-4.59 (m, 1H), 4.44-4.19 (m, 2H), 4.17-4.02 (m, 1H), 3.94 (td, J=10.8, 4.0 Hz, 1H), 3.71 (d, J=6.4 Hz, 1H).

### Example 204

### (7RS)-5-acryloyl-2-(4-fluorophenyl)-3-(pyridin-4-yl)-4,5,6,7-tetrahydropyrazolo[1,5-a]pyrazine-7-carboxamide

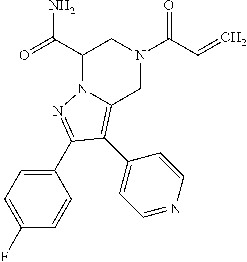 [SMILES: C=CC(=O)N1Cc2c(-c3ccncc3)c(-c3ccc(F)cc3)nn2C(C(N)=O)C1]

To a solution of (7RS)-2-(4-fluorophenyl)-3-(pyridin-4-yl)-4,5,6,7-tetrahydropyrazolo[1,5-a]pyrazine-7-carboxamide (120 mg, 356 μmol, Intermediate 322) in dichloromethane (5 ml) were added trimethylamine (72.0 mg, 711 μmol) and acryloyl chloride (32.2 mg, 356 μmol) at 0° C. The mixture was stirred at 0° C. for 1 hour. The reaction mixture was concentrated in vacuo to give a residue. The residue was purified by preparative HPLC [Instrument: GX-A; Column: Waters Xbridge 150*25 mm*5 μm; eluent A: water (0.2% NH₃′H2O), eluent B: acetonitrile; gradient: 0-10 min 10-35% B; flow 25 ml/min; temperature: RT; Detector: UV 220/254 nm] to give (7RS)-5-acryloyl-2-(4-fluorophenyl)-3-(pyridin-4-yl)-4,5,6,7-tetrahydropyrazolo[1,5-a]pyrazine-7-carboxamide (3.00 mg, 7.10 μmol, 2% yield) as a white solid.

LC-MS (Method 16): Rₜ=0.773 min; MS (ESIpos): m/z=392.2 [M+H]⁺.

¹H NMR (400 MHz, DMSO-d6), δ [ppm]=8.63-8.50 (m, 2H), 7.68-7.55 (m, 1H), 7.42-7.35 (m, 2H), 7.25-7.15 (m, 4H), 6.92-6.76 (m, 1H), 6.21-6.09 (m, 1H), 5.85-5.70 (m, 1H), 5.14 (d, J=17.2 Hz, 1H), 5.07-4.97 (m, 1H), 4.89-4.66 (m, 1H), 4.64-4.43 (m, 2H), 4.19-4.06 (m, 1H).

### Example 205

### 5-acryloyl-2-(4-fluorophenyl)-N-methyl-3-(pyridin-4-yl)-4,5,6,7-tetrahydropyrazolo[1,5-a]pyrazine-7-carboxamide (isomer 1)

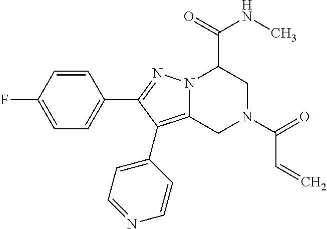 [SMILES: C=CC(=O)N1Cc2c(-c3ccncc3)c(-c3ccc(F)cc3)nn2C(C(=O)NC)C1]

To a solution of (7RS)-2-(4-fluorophenyl)-N-methyl-3-(pyridin-4-yl)-4,5,6,7-tetrahydropyrazolo[1,5-a]pyrazine-7-carboxamide hydrochloride (1:1) (50.0 mg, 129 μmol, Intermediate 324) in dichloromethane (5 ml) were added triethylamine (39.1 mg, 387 μmol) and acryloyl chloride (14.0 mg, 155 μmol) at 0° C. The mixture was stirred at 0° C. for 1 hour. The reaction mixture was concentrated in vacuo to give a residue. The residue was purified by preparative TLC (ethyl acetate:methanol:NH₃·H₂O=10:1:0.1) and preparative HPLC [Instrument: GX-A; Column: Waters Xbridge 150*25 mm*5 μm; eluent A: water (0.2% NH₃′H2O), eluent B: acetonitrile; gradient: 0-10 min 15-45% B; flow 25 ml/min; temperature: RT; Detector: UV 220/254 nm] to give a product. The product was then separated by chiral separation (Column: Chiralpak AD-3 50×4.6 mm I.D., 3 μm Mobile phase: Phase A for CO2, and Phase B for EtOH (0.05% DEA); Gradient elution: EtOH (0.05% DEA) in CO2 from 5% to 40%; Flow rate: 3 mL/min; Detector: PDA; Column Temp: 35 C; Back Pressure: 100 Bar) to give 5-acryloyl-2-(4-fluorophenyl)-N-methyl-3-(pyridin-4-yl)-4,5,6,7-tetrahydropyrazolo[1,5-a]pyrazine-7-carboxamide (isomer 1) (0.800 mg, 1.66 μmol, 84% purity, 1% yield) as a light yellow solid.

LC-MS (Method 16): Rₜ=0.802 min; MS (ESIpos): m/z=406.2 [M+H]⁺

¹H NMR (400 MHz, DMSO-d₆), δ [ppm]=8.56 (s, 2H), 7.37 (d, J=5.6 Hz, 2H), 7.23-7.18 (m, 4H), 6.96-6.65 (m, 1H), 6.13 (dd, J=16.8, 2.0 Hz, 1H), 5.81-5.70 (m, 1H), 5.18-4.97 (m, 2H), 4.69-4.58 (m, 1H), 4.55-4.39 (m, 1H), 4.22-4.08 (m, 1H), 2.63 (d, J=4.4 Hz, 4H).

### Example 206

### 5-acryloyl-2-(4-fluorophenyl)-N,N-dimethyl-3-(pyridin-4-yl)-4,5,6,7-tetrahydropyrazolo[1,5-a]pyrazine-7-carboxamide (isomer 1)

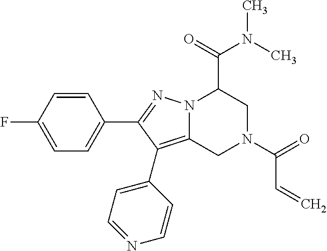 [SMILES: C=CC(=O)N1Cc2c(-c3ccncc3)c(-c3ccc(F)cc3)nn2C(C(=O)N(C)C)C1]

To a solution of (7RS)-2-(4-fluorophenyl)-N,N-dimethyl-3-(pyridin-4-yl)-4,5,6,7-tetrahydropyrazolo[1,5-a]pyrazine-7-carboxamide hydrochloride (1:1) (110 mg, 274 μmol, Intermediate 326) in dichloromethane (2 ml) were added triethylamine (111 mg, 1.09 mmol) and acryloyl chloride (29.7 mg, 328 μmol) at 0° C. The mixture was stirred at 0° C. for 1 hour. The reaction mixture was concentrated in vacuo to give a residue. The residue was purified by preparative TLC (ethyl acetate:methanol:NH₃·H₂O=10:1:0.1) and purified by preparative HPLC [Instrument: GX-A; Column: Waters Xbridge 150*25 mm*5 μm; eluent A: water (0.2% NH3′H2O), eluent B: acetonitrile; gradient: 0-10 min 15-45% B; flow 25 ml/min; temperature: RT; Detector: UV 220/254 nm] to give a racemate. The racemate was separated by SFC chiral separation to give 5-acryloyl-2-(4-fluorophenyl)-N,N-dimethyl-3-(pyridin-4-yl)-4,5,6,7-tetrahydropyrazolo[1,5-a]pyrazine-7-carboxamide (isomer 1) (3.10 mg, 5.21 μmol, 70% purity, 2% yield) as a light yellow solid.

LC-MS (Method 16): R₁=0.824 min; MS (ESIpos): m/z=420.2 [M+H]⁺.

¹H NMR (400 MHz, DMSO-d₆), δ [ppm]=8.56 (s, 2H), 7.39-7.33 (m, 2H), 7.27-7.11 (m, 4H), 6.91-6.73 (m, 1H), 6.20-6.03 (m, 1H), 5.73 (d, J=9.6 Hz, 2H), 5.21-5.07 (m, 1H), 4.68-4.59 (m, 1H), 4.43 (d, J=12.8 Hz, 1H), 4.23-4.13 (m, 1H), 3.27-3.23 (m, 3H), 2.83 (s, 3H).

### Example 207

### 1-[2-(4-fluorophenyl)-7,7-dimethyl-3-(pyridin-4-yl)-6,7-dihydropyrazolo[1,5-a]pyrazin-5(4H)-yl]prop-2-en-1-one

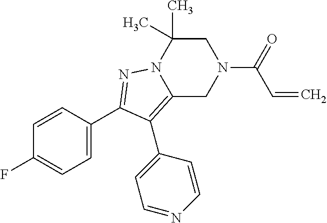 [SMILES: C=CC(=O)N1Cc2c(-c3ccncc3)c(-c3ccc(F)cc3)nn2C(C)(C)C1]

To a solution of 2-(4-fluorophenyl)-7,7-dimethyl-3-(pyridin-4-yl)-4,5,6,7-tetrahydropyrazolo[1,5-a]pyrazine (100 mg, 295 μmol, Intermediate 335) in dichloromethane (2 ml) were added prop-2-enoyl chloride (29.3 mg, 324 μmol) and triethylamine (120 μl, 880 μmol). The mixture was stirred at 25° C. for 30 minutes. The reaction mixture was concentrated in vacuo to give a residue. The residue was purified by preparative HPLC (Instrument: Gilson-281; Column: Waters Xbridge 150*25 mm*5 μm; eluent A: 0.1% ammonia hydroxide in water, eluent B: acetonitrile; gradient: 0-9 min 34-64% B; flow 25 ml/min; temperature: room temperature; Detector: UV 220/254 nm) to give 1-[2-(4-fluorophenyl)-7,7-dimethyl-3-(pyridin-4-yl)-6,7-dihydropyrazolo[1,5-a]pyrazin-5(4H)-yl]prop-2-en-1-one (35.0 mg, 99% purity, 31% yield) as a white solid.

LC-MS (Method 20): Rₜ=0.589 min; MS (ESIpos): m/z=377.4 [M+H]⁺.

¹H NMR (400 MHz, DMSO-d₆) δ [ppm]=8.58-8.50 (m, 2H), 7.42-7.33 (m, 2H), 7.24-7.12 (m, 4H), 7.05-6.82 (m, 1H), 6.30-6.10 (m, 1H), 5.86-5.68 (m, 1H), 5.05-4.74 (m, 2H), 4.10-3.92 (m, 2H), 7.05-6.82 (m, 1H), 1.52 (s, 6H).

### Example 208

### 1-[2-(4-chloro-3-methylphenyl)-7,7-dimethyl-3-(pyridin-4-yl)-6,7-dihydropyrazolo[1,5-a]pyrazin-5(4H)-yl]prop-2-en-1-one

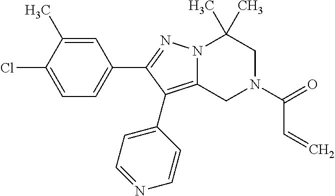 [SMILES: C=CC(=O)N1Cc2c(-c3ccncc3)c(-c3ccc(Cl)c(C)c3)nn2C(C)(C)C1]

To a solution of 2-(4-chloro-3-methylphenyl)-7,7-dimethyl-3-(pyridin-4-yl)-4,5,6,7-tetrahydropyrazolo[1,5-a]pyrazine (30.0 mg, 68.0 μmol, Intermediate 337) in dichloromethane (1.5 ml) were added prop-2-enoyl chloride (6.16 mg, 68.0 μmol) and N,N-diisopropylethylamine (36 μl, 200 μmol) at 25° C. The mixture was stirred at 25° C. for 1 hour. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 44-74% B; flow 25 ml/min; temperature: room temperature; Detector: UV 220/254 nm) to give 1-[2-(4-chloro-3-methylphenyl)-7,7-dimethyl-3-(pyridin-4-yl)-6,7-dihydropyrazolo[1,5-a]pyrazin-5(4H)-yl]prop-2-en-1-one (2.50 mg, 96% purity, 9% yield) as a white solid.

LC-MS (Method 20): Rₜ=0.669 min; MS (ESIpos): m/z=407.4 [M+H]⁺.

¹H NMR (400 MHz, DMSO-d₆) δ [ppm]=8.58-8.48 (m, 2H), 7.45-7.32 (m, 2H), 7.22-7.12 (m, 2H), 7.11-6.82 (m, 2H), 6.32-6.10 (m, 1H), 5.88-5.64 (m, 1H), 5.05-4.75 (m, 2H), 4.15-3.88 (m, 2H), 2.29 (s, 2H), 1.52 (s, 6H).

### Example 209

### 1-[2-(4-chlorophenyl)-7,7-dimethyl-3-(pyridin-4-yl)-6,7-dihydropyrazolo[1,5-a]pyrazin-5(4H)-yl]prop-2-en-1-one

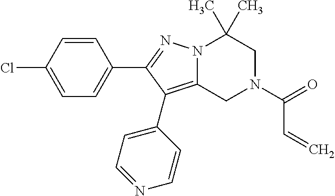 [SMILES: C=CC(=O)N1Cc2c(-c3ccncc3)c(-c3ccc(Cl)cc3)nn2C(C)(C)C1]

To a solution of 2-(4-chlorophenyl)-7,7-dimethyl-3-(pyridin-4-yl)-4,5,6,7-tetrahydropyrazolo[1,5-a]pyrazine (30.0 mg, 69.1 μmol, Intermediate 344) in dichloromethane (3 ml) were added N,N-diisopropylethylamine (36 μl, 210 μmol) and prop-2-enoyl chloride (6.25 mg, 69.1 μmol) at 0° C. The mixture was stirred at 25° C. for 1 hour. The reaction mixture was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 40-70% B; flow 25 ml/min; temperature: room temperature; Detector: UV 220/254 nm) to give 1-[2-(4-chlorophenyl)-7,7-dimethyl-3-(pyridin-4-yl)-6,7-dihydropyrazolo[1,5-a]pyrazin-5(4H)-yl]prop-2-en-1-one (12.0 mg, 99% purity, 44% yield) as a white solid.

LC-MS (Method 20): Rₜ=0.582 min; MS (ESIpos): m/z=439.3 [M+H]⁺.

¹H NMR (400 MHz, DMSO-d₆) δ [ppm]=8.60-8.49 (m, 2H), 7.45-7.40 (m, 2H), 7.39-7.32 (m, 2H), 7.22-7.14 (m, 2H), 7.07-6.80 (m, 1H), 6.31-6.11 (m, 1H), 5.86-5.67 (m, 1H), 5.01-4.80 (m, 2H), 4.15-3.89 (m, 2H), 1.51 (s, 6H).

### Example 210

### 1-[2-(4-fluorophenyl)-6,6-dimethyl-3-(pyridin-4-yl)-6,7-dihydropyrazolo[1,5-a]pyrazin-5(4H)-yl]prop-2-en-1-one

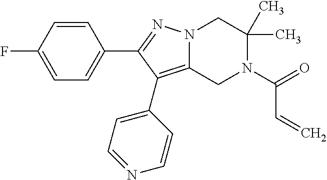 [SMILES: C=CC(=O)N1Cc2c(-c3ccncc3)c(-c3ccc(F)cc3)nn2CC1(C)C]

To a solution of 2-(4-fluorophenyl)-6,6-dimethyl-3-(pyridin-4-yl)-4,5,6,7-tetrahydropyrazolo[1,5-a]pyrazine hydrogen chloride (1:1) (110 mg, 307 μmol, Intermediate 354) and N,N-diisopropylethylamine (160 μl, 920 μmol) in dichloromethane (5 ml) was added prop-2-enoyl chloride (27.7 mg, 307 μmol) at 25° C. and the mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 28-58% B; flow 25 ml/min; temperature: RT; Detector: UV 220/254 nm] to give 1-[2-(4-fluorophenyl)-6,6-dimethyl-3-(pyridin-4-yl)-6,7-dihydropyrazolo[1,5-a]pyrazin-5(4H)-yl]prop-2-en-1-one (30.4 mg, 77.5 μmol, 96% purity, 25% yield) as a white solid.

LC-MS (Method 20): Rₜ=0.874 min; MS (ESIpos): m/z=377.2 [M+1]⁺

¹H NMR (400 MHz, DMSO-d₆) δ [ppm]=8.54 (d, J=6.0 Hz, 2H), 7.39 (dd, J=8.8, 5.6 Hz, 2H), 7.22-7.14 (m, 4H), 6.72 (dd, J=16.8, 10.4 Hz, 1H), 6.02 (dd, J=16.8, 2.0 Hz, 1H), 5.65 (dd, J=10.4, 2.0 Hz, 1H), 4.77 (s, 2H), 4.32 (s, 2H), 1.44 (s, 6H).

### Example 211

### 1-[2-(4-chlorophenyl)-6,6-dimethyl-3-(pyridin-4-yl)-6,7-dihydropyrazolo[1,5-a]pyrazin-5(4H)-yl]prop-2-en-1-one

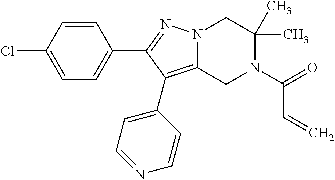 [SMILES: C=CC(=O)N1Cc2c(-c3ccncc3)c(-c3ccc(Cl)cc3)nn2CC1(C)C]

To a solution of 2-(4-chlorophenyl)-6,6-dimethyl-3-(pyridin-4-yl)-4,5,6,7-tetrahydropyrazolo[1,5-a]pyrazine hydrogen chloride (1:1) (100 mg, 266 μmol, Intermediate 356) and N,N-diisopropylethylamine (140 μl, 800 μmol) in dichloromethane (5 ml) was added prop-2-enoyl chloride (36.2 mg, 400 μmol) at 25° C. and the mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 38-68% B; flow 25 ml/min; temperature: RT; Detector: UV 220/254 nm] to give 1-[2-(4-chlorophenyl)-6,6-dimethyl-3-(pyridin-4-yl)-6,7-dihydropyrazolo[1,5-a]pyrazin-5(4H)-yl]prop-2-en-1-one (6.90 mg, 17.0 μmol, 97% purity, 6% yield) as a white solid.

LC-MS (Method 20): Rₜ=0.944 min; MS (ESIpos): m/z=393.1 [M+1]⁺

¹H NMR (400 MHz, DMSO-d6) δ [ppm]=8.60-8.48 (m, 2H), 7.44-7.35 (m, 4H), 7.19-7.14 (m, 2H), 6.71 (dd, J=16.8, 10.4 Hz, 1H), 6.02 (dd, J=16.8, 2.0 Hz, 1H), 5.69-5.61 (m, 1H), 4.77 (s, 2H), 4.33 (s, 2H), 1.44 (s, 6H).

### Example 212

### 1-[2-(4-chloro-3-methylphenyl)-6,6-dimethyl-3-(pyridin-4-yl)-6,7-dihydropyrazolo[1,5-a]pyrazin-5(4H)-yl]prop-2-en-1-one

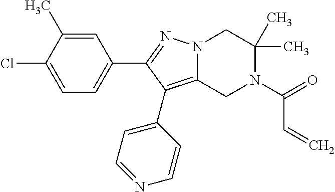 [SMILES: C=CC(=O)N1Cc2c(-c3ccncc3)c(-c3ccc(Cl)c(C)c3)nn2CC1(C)C]

To a solution of 2-(4-chloro-3-methylphenyl)-6,6-dimethyl-3-(pyridin-4-yl)-4,5,6,7-tetrahydropyrazolo[1,5-a]pyrazine hydrogen chloride (1:1) (100 mg, 257 μmol, Intermediate 358) and N,N-diisopropylethylamine (130 μl, 770 μmol) in dichloromethane (5 ml) was added prop-2-enoyl chloride (34.9 mg, 385 μmol) at 25° C. and then the reaction mixture was stirred at 25° C. for 1 hour. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 42-72% B; flow 25 ml/min; temperature: RT; Detector: UV 220/254 nm] to give 1-[2-(4-chloro-3-methylphenyl)-6,6-dimethyl-3-(pyridin-4-yl)-6,7-dihydropyrazolo[1,5-a]pyrazin-5(4H)-yl]prop-2-en-1-one (5.00 mg, 11.8 μmol, 96% purity, 5% yield) as a white solid.

LC-MS (Method 20): Rₜ=0.977 min; MS (ESIpos): m/z=407.1 [M+1]⁺

¹H NMR (400 MHz, DMSO-d₆) δ [ppm]=8.54 (d, J=5.6 Hz, 2H), 7.44 (s, 1H), 7.38 (s, 1H), 7.17 (d, J=5.6 Hz, 2H), 7.08 (d, J=8.4 Hz, 1H), 6.72 (dd, J=16.8, 10.8 Hz, 1H), 6.02 (dd, J=16.4, 1.6 Hz, 1H), 5.69-5.62 (m, 1H), 4.77 (s, 2H), 4.32 (s, 2H), 2.30 (s, 3H), 1.43 (s, 6H).

### Example 213

### (2E)-4-(dimethylamino)-1-[2-(4-fluorophenyl)-7,7-dimethyl-3-(pyridin-4-yl)-6,7-dihydropyrazolo[1,5-a]pyrazin-5(4H)-yl]but-2-en-1-one

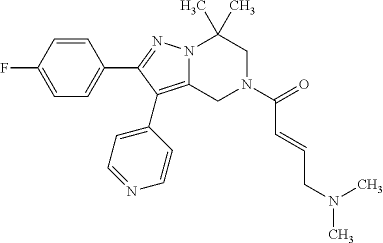 [SMILES: CN(C)C/C=C/C(=O)N1Cc2c(-c3ccncc3)c(-c3ccc(F)cc3)nn2C(C)(C)C1]

To a solution of 2-(4-fluorophenyl)-7,7-dimethyl-3-(pyridin-4-yl)-4,5,6,7-tetrahydropyrazolo[1,5-a]pyrazine (125 mg, 368 μmol, Intermediate 335) in N,N-dimethylformamide (2.0 ml) were added (2E)-4-(dimethylamino)but-2-enoic acid (52.3 mg, 405 μmol), O-(7-azabenzotriazol-1-yl)-N,N,N′,N′-tetramethyluronium hexafluorophosphate (210 mg, 553 μmol) and N,N-diisopropylethylamine (260 μl, 1.5 mmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 34-64% B; flow 25 ml/min; temperature: room temperature; Detector: UV 220/254 nm) to give 1-[2-(4-fluorophenyl)-7,7-dimethyl-3-(pyridin-4-yl)-6,7-dihydropyrazolo[1,5-a]pyrazin-5(4H)-yl]prop-2-en-1-one (5.00 mg, 99% purity, 3% yield) as a white solid.

LC-MS (Method 20): Rₜ=0.606 min; MS (ESIpos): m/z=434.4 [M+H]⁺.

¹H NMR (400 MHz, DMSO-d₆) δ [ppm]=8.60-8.47 (m, 2H), 7.43-7.33 (m, 2H), 7.25-7.11 (m, 4H), 6.86-6.59 (m, 2H), 4.99-4.81 (m, 2H), 4.08-3.91 (m, 2H), 3.09-2.98 (m, 2H), 2.19-2.05 (m, 6H), 1.51 (s, 6H).

### Example 214

### (2E)-1-[2-(4-chloro-3-methylphenyl)-7,7-dimethyl-3-(pyridin-4-yl)-6,7-dihydropyrazolo[1,5-a]pyrazin-5(4H)-yl]-4-(dimethylamino)but-2-en-1-one

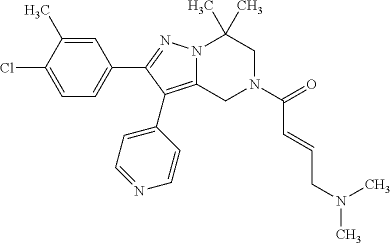 [SMILES: Cc1cc(-c2nn3c(c2-c2ccncc2)CN(C(=O)/C=C/CN(C)C)CC3(C)C)ccc1Cl]

To a solution of 2-(4-chloro-3-methylphenyl)-7,7-dimethyl-3-(pyridin-4-yl)-4,5,6,7-tetrahydropyrazolo[1,5-a]pyrazine (25.0 mg, 56.7 μmol, Intermediate 337) in N,N-dimethylformamide (2.5 ml) were added (2E)-4-(dimethylamino)but-2-enoic acid (8.78 mg, 68.0 μmol), O-(7-azabenzotriazol-1-yl)-N,N,N′,N′-tetramethyluronium hexafluorophosphate (32.3 mg, 85.0 μmol) and N, N-diisopropylethylamine (30 μl, 170 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 45-75% B; flow 25 ml/min; temperature: room temperature; Detector: UV 220/254 nm) to give (2E)-1-[2-(4-chloro-3-methylphenyl)-7,7-dimethyl-3-(pyridin-4-yl)-6,7-dihydropyrazolo[1,5-a]pyrazin-5(4H)-yl]-4-(dimethylamino)but-2-en-1-one (5.00 mg, 94% purity, 18% yield) as a white solid.

LC-MS (Method 20): Rₜ=0.667 min; MS (ESIpos): m/z=464.4 [M+H]⁺

¹H NMR (400 MHz, DMSO-d₆) δ [ppm]=8.60-8.47 (m, 2H), 7.46-7.29 (m, 2H), 7.21-7.12 (m, 4H), 7.11-7.02 (m, 2H), 6.90-6.55 (m, 2H), 5.10-4.71 (m, 2H), 4.25-3.87 (m, 2H), 3.13-2.95 (m, 2H), 2.20-2.04 (m, 6H), 1.52 (s, 6H).

### Example 215

### 1-[(2E)-4-(dimethylamino)but-2-enoyl]-3′-phenyl-2′-(pyridin-4-yl)-5′,6′-dihydrospiro[piperidine-4,7′-pyrrolo[3,2-c]pyridin]-4′ (1′H)-one

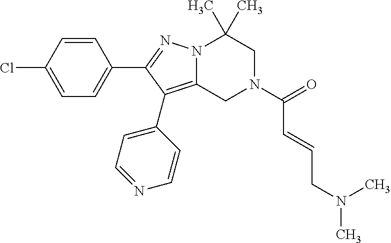 [SMILES: CN(C)C/C=C/C(=O)N1Cc2c(-c3ccncc3)c(-c3ccc(Cl)cc3)nn2C(C)(C)C1]

To a solution of 2-(4-chlorophenyl)-7,7-dimethyl-3-(pyridin-4-yl)-4,5,6,7-tetrahydropyrazolo[1,5-a]pyrazine (60.0 mg, 0.177 mmol, Intermediate 344) and (2E)-4-(dimethylamino)but-2-enoic acid hydrochloride (1:1) (44.0 mg, 0.266 mmol) in N,N-dimethylformamide (2.0 mL) were added 1-[bis(dimethylamino)methylene]-1H-1,2,3-triazolo[4,5-b]pyridinium-3-oxide hexafluorophosphate (101 mg, 0.266 mmol) and N,N-diisopropylethylamine (45.7 mg, 0.354 mmol) at 20° C. The mixture was stirred at 20° C. for 16 hours. The reaction mixture was poured into water and extracted with ethyl acetate. The combined organic layers were washed with brine, dried over anhydrous sodium sulfate, filtered and the filtrate was concentrated in vacuo to give a residue. The residue was purified by preparative HPLC (column: Waters Xbridge 150*25 mm*5 μm; mobile phase: water (ammonia hydroxide v/v)-ACN; B %: 42%-72%, 9 min) to give (2E)-1-[2-(4-chlorophenyl)-7,7-dimethyl-3-(pyridin-4-yl)-6,7-dihydropyrazolo[1,5-a]pyrazin-5(4H)-yl]-4-(dimethylamino)but-2-en-1-one (9.70 mg, 0.0207 mmol, 12% yield) as a white solid.

LC-MS (Method 20): Rₜ=1.013 min; MS (ESIpos): m/z=449.2 [M+H]⁺.

¹H NMR (400 MHz, DMSO-d₆) δ [ppm]=8.55 (d, J=4.8 Hz, 2H), 7.45-7.39 (m, 2H), 7.39-7.32 (m, 2H), 7.17 (d, J=4.0 Hz, 2H), 6.85-6.59 (m, 2H), 4.99-4.77 (m, 2H), 4.11-3.89 (m, 3H), 3.10-2.97 (m, 3H), 2.16-2.15 (m, 1H), 2.20-2.06 (m, 7H), 1.52 (s, 6H).

### Example 216

### (2E)-4-(dimethylamino)-1-[2-(4-fluorophenyl)-6,6-dimethyl-3-(pyridin-4-yl)-6,7-dihydropyrazolo[1,5-a]pyrazin-5(4H)-yl]but-2-en-1-one

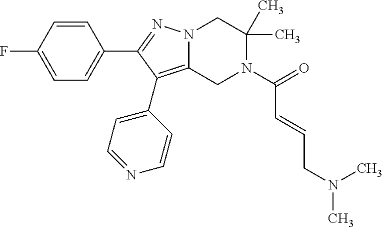 [SMILES: CN(C)C/C=C/C(=O)N1Cc2c(-c3ccncc3)c(-c3ccc(F)cc3)nn2CC1(C)C]

To a solution of (2E)-4-(dimethylamino)but-2-enoic acid hydrogen chloride (1:1) (48.5 mg, 293 μmol) and N,N-diisopropylethylamine (140 μl, 780 μmol) in N,N-dimethylformamide (4 ml) was added 1-[bis(dimethylamino)methylene]-1H-1,2,3-triazolo[4,5-b]pyridinium-3-oxide hexafluorophosphate (111 mg, 293 μmol) at 25° C. and the mixture was stirred at 25° C. for 10 minutes. To the mixture above was added 2-(4-fluorophenyl)-6,6-dimethyl-3-(pyridin-4-yl)-4,5,6,7-tetrahydropyrazolo[1,5-a]pyrazine hydrogen chloride (1:1) (70.0 mg, 195 μmol, Intermediate 354) and the resulting mixture was stirred at 80° C. for 2 hours. The reaction mixture was poured into water and extracted with ethyl acetate. The combined extracts were concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 20-50% B; flow 25 ml/min; temperature: RT; Detector: UV 220/254 nm] to give (2E)-4-(dimethylamino)-1-[2-(4-fluorophenyl)-6,6-dimethyl-3-(pyridin-4-yl)-6,7-dihydropyrazolo[1,5-a]pyrazin-5(4H)-yl]but-2-en-1-one (2.30 mg, 4.66 μmol, 88% purity, 2% yield) as a brown solid.

LC-MS (Method 20): Rₜ=0.900 min; MS (ESIpos): m/z=434.2 [M+1]⁺

¹H NMR (400 MHz, DMSO-d₆) δ [ppm]=8.56-8.45 (m, 2H), 7.43-7.34 (m, 2H), 7.19 (t, J=8.8 Hz, 2H), 7.16-7.12 (m, 2H), 6.50-6.42 (m, 2H), 4.75 (s, 2H), 4.31 (s, 2H), 2.96 (d, J=4.4 Hz, 2H), 2.08-2.05 (m, 6H), 1.42 (s, 6H).

### Example 217

### (2E)-1-[2-(4-chlorophenyl)-6,6-dimethyl-3-(pyridin-4-yl)-6,7-dihydropyrazolo[1,5-a]pyrazin-5(4H)-yl]-4-(dimethylamino)but-2-en-1-one

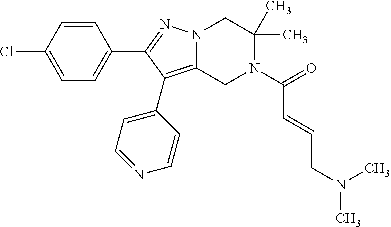 [SMILES: CN(C)C/C=C/C(=O)N1Cc2c(-c3ccncc3)c(-c3ccc(Cl)cc3)nn2CC1(C)C]

To a solution of (2E)-4-(dimethylamino)but-2-enoic acid hydrogen chloride (1:1) (97.1 mg, 586 μmol) and N,N-diisopropylethylamine (200 μl, 1.20 mmol) in N,N-dimethylformamide was (6 ml) was added 1-[bis(dimethylamino)methylene]-1H-1,2,3-triazolo[4,5-b]pyridinium-3-oxide hexafluorophosphateb (279 mg, 733 μmol) at 25° C. and the mixture was stirred at 25° C. for 10 minutes. To the mixture above was added 2-(4-chlorophenyl)-6,6-dimethyl-3-(pyridin-4-yl)-4,5,6,7-tetrahydropyrazolo[1,5-a]pyrazine hydrogen chloride (1:1) (110 mg, 293 μmol, Intermediate 356) and the resulting mixture was stirred at 80° C. for 16 hours. The reaction mixture was poured into water and extracted with ethyl acetate. The combined extracts were concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 20-50% B; flow 25 ml/min; temperature: RT; Detector: UV 220/254 nm] to give 2E)-1-[2-(4-chlorophenyl)-6,6-dimethyl-3-(pyridin-4-yl)-6,7-dihydropyrazolo[1,5-a]pyrazin-5(4H)-yl]-4-(dimethylamino)but-2-en-1-one (3.60 mg, 7.28 μmol, 91% purity, 2% yield) as a brown solid.

LC-MS (Method 20): Rₜ=0.980 min; MS (ESIpos): m/z=450.2 [M+1]⁺

¹H NMR (400 MHz, DMSO-d₆) δ [ppm]=8.53 (d, J=6.0 Hz, 2H), 7.48-7.33 (m, 4H), 7.17 (d, J=6.0 Hz, 2H), 6.50-6.40 (m, 2H), 4.76 (s, 2H), 4.32 (s, 2H), 2.97 (d, J=4.4 Hz, 2H), 2.08 (s, 6H), 1.43 (s, 6H).

### Example 218

### (2E)-1-[2-(4-chloro-3-methylphenyl)-6,6-dimethyl-3-(pyridin-4-yl)-6,7-dihydropyrazolo[1,5-a]pyrazin-5(4H)-yl]-4-(dimethylamino)but-2-en-1-one

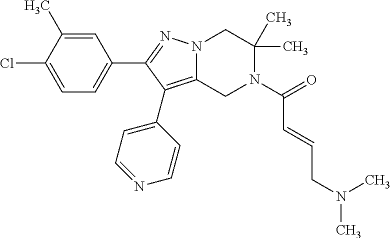 [SMILES: Cc1cc(-c2nn3c(c2-c2ccncc2)CN(C(=O)/C=C/CN(C)C)C(C)(C)C3)ccc1Cl]

To a solution of (2E)-4-(dimethylamino)but-2-enoic acid hydrogen chloride (1:1) (102 mg, 616 μmol) and N,N-diisopropylethylamine (210 μl, 1.2 mmol) in N,N-dimethylformamide (6 ml) was added 1-[bis(dimethylamino)methylene]-1H-1,2,3-triazolo[4,5-b]pyridinium-3-oxide hexafluorophosphate (293 mg, 771 μmol) at 25° C. and the mixture was stirred at 25° C. for 10 minutes. To the mixture above was added 2-(4-chloro-3-methylphenyl)-6,6-dimethyl-3-(pyridin-4-yl)-4,5,6,7-tetrahydropyrazolo[1,5-a]pyrazine hydrogen chloride (1:1) (120 mg, 308 μmol, Intermediate 358) and the resulting mixture was stirred at 80° C. for 2 hours. The reaction mixture was poured into water and extracted with ethyl acetate. The combined extracts were concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 20-50% B; flow 25 ml/min; temperature: RT; Detector: UV 220/254 nm] to give (2E)-1-[2-(4-chloro-3-methylphenyl)-6,6-dimethyl-3-(pyridin-4-yl)-6,7-dihydropyrazolo[1,5-a]pyrazin-5(4H)-yl]-4-(dimethylamino)but-2-en-1-one (3.20 mg, 6.69 μmol, 97% purity, 2% yield) as a brown solid.

LC-MS (Method 20): Rₜ=1.013 min; MS (ESIpos): m/z=464.2 [M+1]⁺

¹H NMR (400 MHz, DMSO-d₆) δ [ppm]=8.59-8.46 (m, 2H), 7.44 (d, J=1.6 Hz, 1H), 7.36 (d, J=8.4 Hz, 1H), 7.19-7.14 (m, 2H), 7.09 (dd, J=8.4, 2.0 Hz, 1H), 6.51-6.39 (m, 2H), 4.76 (s, 2H), 4.32 (s, 2H), 2.97 (d, J=4.4 Hz, 2H), 2.29 (s, 3H), 2.08 (s, 6H), 1.42 (s, 6H).

### Example 219

### rac-(R,E)-4-(dimethylamino)-1-(2-(4-fluorophenyl)-7-(2-hydroxyethyl)-3-(pyridin-4-yl)-6,7-dihydropyrazolo[1,5-a]pyrazin-5(4H)-yl) but-2-en-1-one

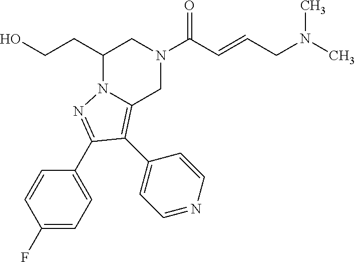 [SMILES: CN(C)C/C=C/C(=O)N1Cc2c(-c3ccncc3)c(-c3ccc(F)cc3)nn2C(CCO)C1]

To a solution of 2-[(7RS)-2-(4-fluorophenyl)-3-(pyridin-4-yl)-4,5,6,7-tetrahydropyrazolo[1,5-a]pyrazin-7-yl]ethan-1-ol hydrogen chloride (1/1) (328 mg, 90% purity, 872 μmol, Intermediate 369) and (2E)-4-(dimethylamino)but-2-enoic acid hydrogen chloride (1/1) (124 mg, 960 μmol) in N,N-dimethylformamide (18 ml) were added bis(2-oxo-3-oxazolidinyl)phosphinic chloride (333 mg, 1.31 mmol) and N,N-diisopropylethylamine (610 μl, 3.5 mmol) at 25° C. The mixture was stirred at 25° C. for 2 hours. The reaction was quenched with water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give rac-(R,E)-4-(dimethylamino)-1-(2-(4-fluorophenyl)-7-(2-hydroxyethyl)-3-(pyridin-4-yl)-6,7-dihydropyrazolo[1,5-a]pyrazin-5(4H)-yl) but-2-en-1-one (19.0 mg, 97% purity, 5% yield) as a colorless oil.

LC-MS (Method 20): Rₜ=0.885 min; MS (ESIpos): m/z=450.4 [M+H]⁺.

¹H NMR (400 MHz, DMSO-d₆) δ [ppm]=8.54 (d, J=4.8 Hz, 2H), 7.37 (d, J=1.6 Hz, 2H), 7.22-7.12 (m, 4H), 6.80-6.54 (m, 2H), 5.10-4.90 (m, 1H), 4.90-4.60 (m, 2H), 4.49 (dd, J=7.6, 4.0 Hz, 1H), 4.40-3.90 (m, 2H), 3.75-3.59 (m, 2H), 3.10-2.97 (m, 2H), 2.15 (s, 6H), 2.08-1.68 (m, 2H).

### Example 220

### 2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]acetamide

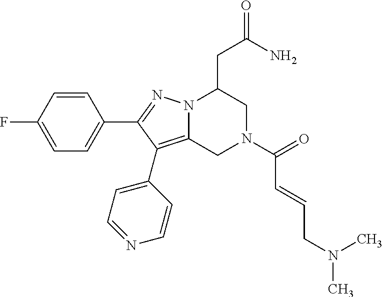 [SMILES: CN(C)C/C=C/C(=O)N1Cc2c(-c3ccncc3)c(-c3ccc(F)cc3)nn2C(CC(N)=O)C1]

To a solution of 2-[(7RS)-2-(4-fluorophenyl)-3-(pyridin-4-yl)-4,5,6,7-tetrahydropyrazolo[1,5-a]pyrazin-7-yl]acetamide hydrogen chloride (1/1) (35.0 mg, 53% purity, 47.8 μmol, Intermediate 373) and (2E)-4-(dimethylamino)but-2-enoic acid hydrogen chloride (1/1) (6.80 mg, 52.6 μmol) in N,N-dimethylformamide (1.0 ml) were added O-(7-azabenzotriazol-1-yl)-N,N,N′,N′-tetramethyluronium hexafluorophosphate (27.3 mg, 71.7 μmol) and N,N-diisopropylethylamine (33 μl, 190 μmol) at 25° C. The mixture was stirred at 25° C. for 2 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to 2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]acetamide (3.60 mg, 98% purity, 16% yield) as a colorless oil.

LC-MS (Method 20): Rₜ=0.824 min; MS (ESIpos): m/z=463.1 [M+H]⁺.

¹H NMR (400 MHz, DMSO-d6) δ [ppm]=8.58 (d, J=1.2 Hz, 2H), 7.37 (dd, J=8.4, 5.6 Hz, 4H), 7.08 (d, J=5.6 Hz, 2H), 7.02 (t, J=8.4 Hz, 4H), 5.51-5.39 (m, 1H), 4.89-4.67 (m, 2H), 3.21-3.14 (m, 2H), 2.77 (s, 1H), 2.36-2.28 (m, 6H), 2.03-2.00 (m, 2H).

### Example 221

### 2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N-methylacetamide

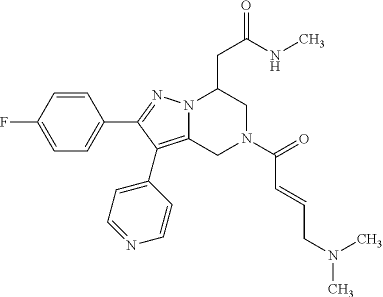 [SMILES: CNC(=O)CC1CN(C(=O)/C=C/CN(C)C)Cc2c(-c3ccncc3)c(-c3ccc(F)cc3)nn21]

To a solution of 2-[(7RS)-2-(4-fluorophenyl)-3-(pyridin-4-yl)-4,5,6,7-tetrahydropyrazolo[1,5-a]pyrazin-7-yl]-N-methylacetamide hydrogen chloride (1/1) (61.0 mg, 152 μmol Intermediate 375) and (2E)-4-(dimethylamino)but-2-enoic acid (21.6 mg, 167 μmol) in N,N-dimethylformamide (1.0 ml) were added O-(7-azabenzotriazol-1-yl)-N,N,N′,N′-tetramethyluronium hexafluorophosphate (86.6 mg, 228 μmol) and N,N-diisopropylethylamine (110 μl, 610 μmol) at 25° C. The mixture was stirred at 25° C. for 2 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N-methylacetamide (4.60 mg, 96% purity, 6% yield) as a colorless oil.

LC-MS (Method 20): Rₜ=0.847 min; MS (ESIpos): m/z=477.1 [M+H]⁺.

¹H NMR (400 MHz, DMSO-d₆) δ [ppm]=8.55 (d, J=5.2 Hz, 2H), 8.01-7.92 (m, 1H), 7.35 (d, J=5.6 Hz, 2H), 7.24-7.18 (m, 2H), 7.16 (d, J=5.2 Hz, 2H), 6.66 (s, 2H), 4.94 (d, J=16.0 Hz, 1H), 4.73-4.61 (m, 2H), 4.35 (t, J=5.2 Hz, 1H), 4.18 (d, J=9.6 Hz, 1H), 3.03 (s, 3H), 2.89 (dd, J=15.2, 4.4 Hz, 1H), 2.63 (d, J=4.4 Hz, 3H), 2.14 (s, 6H).

### Example 222

### 2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N,N-dimethylacetamide

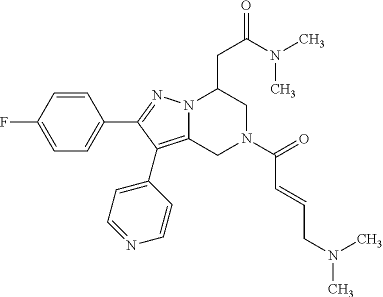 [SMILES: CN(C)C/C=C/C(=O)N1Cc2c(-c3ccncc3)c(-c3ccc(F)cc3)nn2C(CC(=O)N(C)C)C1]

To a solution of 2-[(7RS)-2-(4-fluorophenyl)-3-(pyridin-4-yl)-4,5,6,7-tetrahydropyrazolo[1,5-a]pyrazin-7-yl]-N,N-dimethylacetamide hydrogen chloride (1/1) (61.0 mg, 147 μmol, Intermediate 377) and (2E)-4-(dimethylamino)but-2-enoic acid (20.8 mg, 161 μmol) in N,N-dimethylformamide (2.0 ml) were added O-(7-azabenzotriazol-1-yl)-N,N,N′,N′-tetramethyluronium (83.7 μmol) hexafluorophosphate mg, 220 and N,N-diisopropylethylamine (100 μl, 590 μmol) at 25° C. The mixture was stirred at 25° C. for 2 hours. The reaction was quenched with water and the resulting mixture was extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N,N-dimethylacetamide (16.2 mg, 97% purity, 22% yield) as a colourless oil.

LC-MS (Method 22): Rₜ=0.915 min; MS (ESIpos): m/z=491.4 [M+H]⁺

¹H NMR (400 MHz, DMSO-d₆) δ [ppm]=8.55 (d, J=5.6 Hz, 2H), 7.38 (t, J=5.6 Hz, 2H), 7.22-7.15 (m, 4H), 6.71-6.50 (m, 2H), 4.99 (d, J=16.4, 1H), 4.90-4.66 (m, 2H), 4.25-3.92 (m, 2H), 3.11-3.01 (m, 1H), 3.04-2.98 (m, 2H), 2.92 (s, 3H), 2.88 (s, 3H), 2.78-2.71 (m, 1H), 2.14 (s, 6H).

### Example 223

### 1-(2-(4-fluorophenyl)-7-(2-hydroxyethyl)-3-(pyridin-4-yl)-6,7-dihydropyrazolo[1,5-a]pyrazin-5(4H)-yl) prop-2-en-1-one

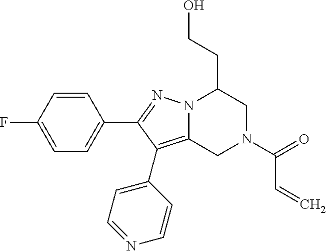 [SMILES: C=CC(=O)N1Cc2c(-c3ccncc3)c(-c3ccc(F)cc3)nn2C(CCO)C1]

To a solution of 2-[2-(4-fluorophenyl)-3-(pyridin-4-yl)-4,5,6,7-tetrahydropyrazolo[1,5-a]pyrazin-7-yl]ethan-1-ol hydrogen chloride (1/1) (100 mg, 267 μmol, Intermediate 369) and prop-2-enoyl chloride (26.6 mg, 293 μmol) in dichloromethane (4.0 ml) was added N,N-diisopropylethylamine (190 μl, 1.10 mmol) at 25° C. The mixture was stirred at 25° C. for 2 hours. The reaction was quenched with water and the resulting mixture was extracted with ethyl acetate. The combined organic phase was washed with brine, dried over anhydrous sodium sulfate, filtered and concentrated in vacuo to a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 1-(2-(4-fluorophenyl)-7-(2-hydroxyethyl)-3-(pyridin-4-yl)-6,7-dihydropyrazolo[1,5-a]pyrazin-5(4H)-yl) prop-2-en-1-one (10.9 mg 97% purity, 10% yield) as a colourless oil.

LC-MS (Method 20): Rₜ=0.803 min; MS (ESIpos): m/z=393.2 [M+H]⁺.

¹H NMR (400 MHz, DMSO-d₆) δ [ppm]=8.54 (s, 2H), 7.37 (s, 2H), 7.19 (s, 4H), 7.05-6.89 (m, 1H), 6.36-6.09 (m, 1H), 5.90-5.65 (m, 1H), 5.18-4.92 (m, 1H), 4.91-4.67 (m, 2H), 4.59-4.44 (m, 1H), 4.36-3.79 (m, 2H), 3.77-3.59 (m, 2H), 2.21-2.12 (m, 1H), 1.92-1.77 (m, 1H).

### Example 224

### 2-(5-acryloyl-2-(4-fluorophenyl)-3-(pyridin-4-yl)-4,5,6,7-tetrahydropyrazolo[1,5-a]pyrazin-7-yl) acetamide

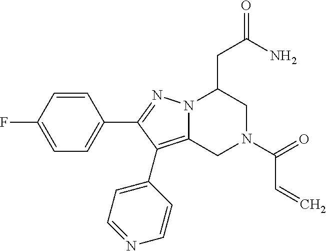 [SMILES: C=CC(=O)N1Cc2c(-c3ccncc3)c(-c3ccc(F)cc3)nn2C(CC(N)=O)C1]

To a solution of 2-(2-(4-fluorophenyl)-3-(pyridin-4-yl)-4,5,6,7-tetrahydropyrazolo[1,5-a]pyrazin-7-yl) acetamide hydrochloride (1:1) (80.0 mg, 41% purity, 84.6 μmol, Intermediate 373) and acryloyl chloride (8.42 mg, 93.0 μmol) in dichloromethane (2.0 ml) was added N,N-diisopropylethylamine (0.0590 ml, 338 μmol) at 0° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 2-(5-acryloyl-2-(4-fluorophenyl)-3-(pyridin-4-yl)-4,5,6,7-tetrahydropyrazolo[1,5-a]pyrazin-7-yl) acetamide (2.60 mg 96% purity, 7% yield) as a colourless oil.

LC-MS (Method 22): Rₜ=0.860 min; MS (ESIpos): m/z=406.2 [M+H]⁺.

¹H NMR (400 MHz, DMSO-d₆) δ [ppm]=8.59-8.52 (m, 2H), 7.45-7.30 (m, 3H), 7.29-7.14 (m, 5H), 7.13-6.83 (m, 1H), 6.31-6.12 (m, 1H), 5.87-5.73 (m, 1H), 5.01-4.86 (m, 1H), 4.82-4.62 (m, 2H), 4.25-4.05 (m, 2H), 3.01-2.84 (m, 2H).

### Example 225

### 2-(5-acryloyl-2-(4-fluorophenyl)-3-(pyridin-4-yl)-4,5,6,7-tetrahydropyrazolo[1,5-a]pyrazin-7-yl)-N-methylacetamide

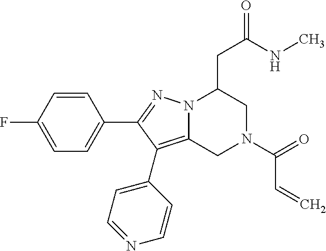 [SMILES: C=CC(=O)N1Cc2c(-c3ccncc3)c(-c3ccc(F)cc3)nn2C(CC(=O)NC)C1]

To a solution of 2-(2-(4-fluorophenyl)-3-(pyridin-4-yl)-4,5,6,7-tetrahydropyrazolo[1,5-a]pyrazin-7-yl)-N-methylacetamide hydrochloride (1:1) (65.0 mg, 63% purity, 102 μmol, Intermediate 375) and acryloyl chloride (10.1 mg, 112 μmol) in dichloromethane (1.3 ml) was added N,N-diisopropylethylamine (71 μl, 410 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 2-(5-acryloyl-2-(4-fluorophenyl)-3-(pyridin-4-yl)-4,5,6,7-tetrahydropyrazolo[1,5-a]pyrazin-7-yl)-N-methylacetamide (2.00 mg, 95% purity, 4% yield) as a colourless oil.

LC-MS (Method 21): Rₜ=0.838 min; MS (ESIpos): m/z=420.0 [M+H]⁺.

¹H NMR (400 MHz, DMSO-d₆), δ [ppm]=8.54 (s, 2H), 8.04 (s, 1H), 7.42-7.32 (m, 2H), 7.23-7.16 (m, 4H), 6.88 (dd, J=16.4, 10.4 Hz, 1H), 6.28-6.11 (m, 1H), 5.87-5.65 (m, 1H), 5.05-4.90 (m, 1H), 4.80-4.65 (m, 2H), 4.26-4.06 (m, 2H), 3.04-2.86 (m, 2H), 2.63 (d, J=4.0 Hz, 3H).

### Example 226

### 2-(5-acryloyl-2-(4-fluorophenyl)-3-(pyridin-4-yl)-4,5,6,7-tetrahydropyrazolo[1,5-a]pyrazin-7-yl)-N,N-dimethylacetamide

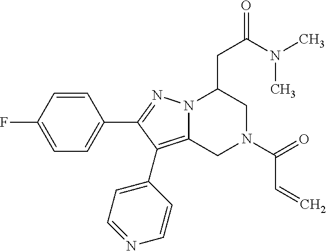 [SMILES: C=CC(=O)N1Cc2c(-c3ccncc3)c(-c3ccc(F)cc3)nn2C(CC(=O)N(C)C)C1]

To a solution of 2-(2-(4-fluorophenyl)-3-(pyridin-4-yl)-4,5,6,7-tetrahydropyrazolo[1,5-a]pyrazin-7-yl)-N,N-dimethylacetamide hydrochloride (1:1) (80.0 mg, 57% purity, 110 μmol, Intermediate 377) and acryloyl chloride (10.9 mg, 121 μmol) in dichloromethane (1.1 ml) was added N,N-diisopropylethylamine (76 μl, 440 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 2-(5-acryloyl-2-(4-fluorophenyl)-3-(pyridin-4-yl)-4,5,6,7-tetrahydropyrazolo[1,5-a]pyrazin-7-yl)-N,N-dimethylacetamide (4.20 mg, 91% purity, 8% yield) as a colourless oil.

LC-MS (Method 19): Rₜ=0.512 min; MS (ESIpos): m/z=434.4 [M+H]⁺.

¹H NMR (400 MHz, DMSO-d6), δ [ppm]=8.55 (s, 2H), 7.37 (d, J=5.6 Hz, 2H), 7.23-7.15 (m, 4H), 6.94-6.66 (m, 1H), 6.27-6.05 (m, 1H), 5.86-5.67 (m, 1H), 5.09-5.85 (m, 1H), 4.83-4.66 (m, 2H), 4.37-3.85 (m, 2H), 2.93 (s, 3H), 2.88 (s, 3H), 2.75 (dd, J=16.4, 10.4 Hz, 2H).

### Example 227

### 1-(2-(4-chloro-2-fluorophenyl)-6-methyl-3-(pyridin-4-yl)-6,7-dihydropyrazolo[1,5-a]pyrazin-5(4H)-yl) prop-2-en-1-one

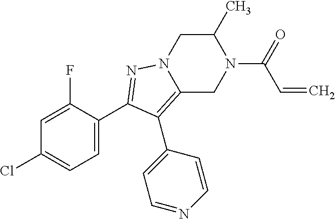 [SMILES: C=CC(=O)N1Cc2c(-c3ccncc3)c(-c3ccc(Cl)cc3F)nn2CC1C]

To a solution of 2-(4-chloro-2-fluorophenyl)-6-methyl-3-(pyridin-4-yl)-4,5,6,7-tetrahydropyrazolo[1,5-a]pyrazine hydrogen chloride (1/1) (80.0 mg, 89% purity, 188 μmol, Intermediate 385) and prop-2-enoyl chloride (18.7 mg, 207 μmol) in dichloromethane (2.0 ml) was added triethylamine (78 μl, 560 μmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give 1-(2-(4-chloro-2-fluorophenyl)-6-methyl-3-(pyridin-4-yl)-6,7-dihydropyrazolo[1,5-a]pyrazin-5(4H)-yl) prop-2-en-1-one (7.50 mg, 95% purity, 18.8 μmol, 10% yield) as a colourless oil.

LC-MS (Method 20): Rₜ=0.907 min; MS (ESIpos): m/z=397.1 [M+H]⁺.

¹H NMR (400 MHz, DMSO-d₆), δ [ppm]=8.5 (d, J=5.6 Hz, 2H), 7.56-7.50 (m, 1H), 7.46 (dd, J=9.6, 2.0 Hz, 1H), 7.38 (dd, J=8.8, 1.6 Hz, 1H), 7.14 (d, J=5.6 Hz, 2H), 7.00-6.84 (m, 1H), 6.18 (dd, J=16.4, 2.0 Hz, 1H), 5.77 (d, J=11.2 Hz, 1H), 5.23 (d, J=17.2 Hz, 1H) 5.10-4.80 (m, 1H), 4.77-4.44 (m, 1H), 4.37-4.23 (m, 2H)), 1.23 (s, 3H).

### Example 228

### 1-[(6RS)-2-(4-methoxyphenyl)-6-methyl-3-(pyridin-4-yl)-6,7-dihydropyrazolo[1,5-a]pyrazin-5(4H)-yl]prop-2-en-1-one

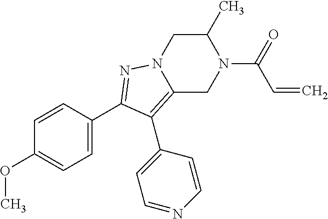 [SMILES: C=CC(=O)N1Cc2c(-c3ccncc3)c(-c3ccc(OC)cc3)nn2CC1C]

To a solution of 2-(4-methoxyphenyl)-6-methyl-3-(pyridin-4-yl)-4,5,6,7-tetrahydropyrazolo[1,5-a]pyrazine hydrogen chloride (1/1) (110 mg, 97% purity, 299 μmol, Intermediate 390) and prop-2-enoyl chloride (40.6 mg, 448 μmol) in dichloromethane (2.0 ml) was added triethylamine (130 μl, 900 μmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give 1-(2-(4-methoxyphenyl)-6-methyl-3-(pyridin-4-yl)-6,7-dihydropyrazolo[1,5-a]pyrazin-5(4H)-yl) prop-2-en-1-one (30.1 mg, 98% purity, 26% yield) as a colourless oil.

LC-MS (Method 20): Rₜ=0.855 min; MS (ESIpos): m/z=375.2 [M+H]⁺.

¹H NMR (400 MHz, DMSO-d₆), δ [ppm]=8.53 (d, J=6.0 Hz, 2H), 7.28 (d, J=8.8 Hz, 2H), 7.22 (d, J=6.0 Hz, 2H), 6.91 (d, J=8.8 Hz, 3H), 6.17 (dd, J=16.8, 2.0 Hz, 1H), 5.76 (d, J=9.6 Hz, 1H), 5.13 (d, J=16.8 Hz, 1H), 5.07-4.71 (m, 1H), 4.69-4.36 (m, 1H) 4.35-4.16 (m, 2H), 3.76 (s, 3H), 1.24 (s, 3H).

### Example 229

### 1-[(RS)-2-(2-fluoro-1-benzothiophen-6-yl)-6-methyl-3-(pyridin-4-yl)-6,7-dihydropyrazolo[1,5-a]pyrazin-5(4H)-yl]prop-2-en-1-one

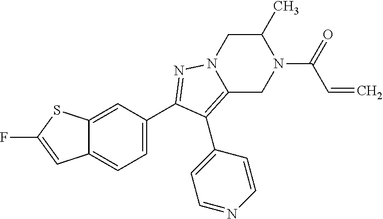 [SMILES: C=CC(=O)N1Cc2c(-c3ccncc3)c(-c3ccc4cc(F)sc4c3)nn2CC1C]

To a solution of (RS)-2-(2-fluoro-1-benzothiophen-6-yl)-6-methyl-3-(pyridin-4-yl)-4,5,6,7-tetrahydropyrazolo[1,5-a]pyrazine hydrogen chloride (1/1) (100 mg, 65% purity, 162 μmol, Intermediate 394) in dichloromethane (2.0 ml) were added prop-2-enoyl chloride (14.7 mg, 162 μmol) and triethylamine (68 μl, 490 μmol) at 25° C. The mixture was stirred at 25° C. for 30 minutes. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex C18 75*30 mm*3 μm; eluent A: 0.1% formic acid in water, eluent B: acetonitrile; gradient: 0-9 min 14-44% B; flow 25 ml/min; temperature: room temperature; Detector: UV 220/254 nm) and preparative TLC (ethyl acetate:methanol=30:1) to give 1-[(RS)-2-(2-fluoro-1-benzothiophen-6-yl)-6-methyl-3-(pyridin-4-yl)-6,7-dihydropyrazolo[1,5-a]pyrazin-5(4H)-yl]prop-2-en-1-one (8.60 mg, 98% purity, 12% yield) as a white solid.

LC-MS (Method 16): Rₜ=0.943 min; MS (ESIpos): m/z=419.2 [M+H]⁺.

¹H NMR (400 MHz, DMSO-d₆), δ [ppm]=8.58-8.45 (m, 2H), 7.96 (s, 1H), 7.72-7.61 (m, 1H), 7.34-7.27 (m, 1H), 7.26-7.18 (m, 2H), 7.10 (d, J=2.8 Hz, 1H), 7.01-6.80 (m, 1H), 6.24-6.11 (m, 1H), 5.82-5.70 (m, 1H), 5.22-5.12 (m, 1H), 5.08-4.73 (m, 1H), 4.72-4.38 (m, 1H), 4.37-4.19 (m, 2H), 1.29-1.18 (m, 3H).

### Example 230

### 1-[(6RS)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one

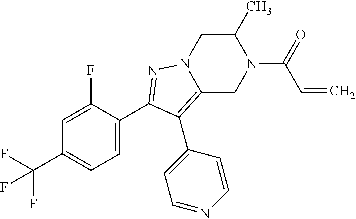 [SMILES: C=CC(=O)N1Cc2c(-c3ccncc3)c(-c3ccc(C(F)(F)F)cc3F)nn2CC1C]

To a solution of (RS)-2-[2-fluoro-4-(trifluoromethyl)phenyl]-6-methyl-3-(pyridin-4-yl)-4,5,6,7-tetrahydropyrazolo[1,5-a]pyrazine hydrogen chloride (1/1) (150 mg, 98% purity, 356 μmol, Intermediate 396) and prop-2-enoyl chloride (48.3 mg, 534 μmol) in dichloromethane (2.0 ml) was added triethylamine (150 μl, 1.10 mmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give (RS)-1-(2-(2-fluoro-4-(trifluoromethyl)phenyl)-6-methyl-3-(pyridin-4-yl)-6,7-dihydropyrazolo[1,5-a]pyrazin-5(4H)-yl) prop-2-en-1-one (49.7 mg, 97% purity, 31% yield) as a colourless oil.

LC-MS (Method 20): Rₜ=0.915 min; MS (ESIpos): m/z=431.1 [M+H]⁺.

¹H NMR (400 MHz, DMSO-d6), δ [ppm]=8.52 (d, J=6.0 Hz, 2H), 7.8-7.74 (m, 1H), 7.73-7.66 (m, 2H), 7.16 (d, J=6.0 Hz, 2H), 7.00-6.80 (m, 1H), 6.18 (dd, J=16.8, 2.4 Hz, 1H), 5.77 (dd, J=10.4, 1.6 Hz, 1H), 5.24 (d, J=17.2 Hz, 1H), 5.17-4.83 (m, 1H) 4.82-4.43 (m, 1H), 4.43-4.15 (m, 2H), 1.24 (s, 3H).

### Example 231

### 1-[(RS)-2-(2-fluoro-4-methoxyphenyl)-6-methyl-3-(pyridin-4-yl)-6,7-dihydropyrazolo[1,5-a]pyrazin-5(4H)-yl]prop-2-en-1-one

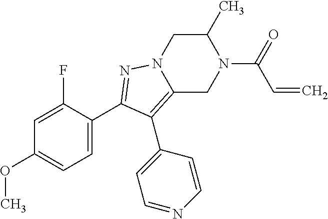 [SMILES: C=CC(=O)N1Cc2c(-c3ccncc3)c(-c3ccc(OC)cc3F)nn2CC1C]

To a solution of (RS)-2-(2-fluoro-4-methoxyphenyl)-6-methyl-3-(pyridin-4-yl)-4,5,6,7-tetrahydropyrazolo[1,5-a]pyrazine hydrogen chloride (1/1) (130 mg, 96% purity, 333 μmol, Intermediate 398) and prop-2-enoyl chloride (45.2 mg, 499 μmol) in dichloromethane (2.0 ml) was added triethylamine (140 μl, 1.00 mmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give (RS)-1-(2-(2-fluoro-4-methoxyphenyl)-6-methyl-3-(pyridin-4-yl)-6,7-dihydropyrazolo[1,5-a]pyrazin-5(4H)-yl) prop-2-en-1-one (46.7 mg, 98% purity, 35% yield) as a colourless oil.

LC-MS (Method 20): Rₜ=0.848 min; MS (ESIpos): m/z=393.1 [M+H]⁺.

¹H NMR (400 MHz, DMSO-d₆), δ [ppm]=8.48 (d, J=6.0 Hz, 2H), 7.43-7.35 (m, 1H), 7.13 (d, J=6.0 Hz, 2H), 6.91-6.88 (m, 1H), 6.87-6.81 (m, 2H), 6.18 (dd, J=16.8, 2.0 Hz, 1H), 5.77 (d, J=11.6 Hz, 1H), 5.22 (d, J=17.6 Hz, 1H), 5.15-4.76 (m, 1H) 4.75-4.36 (m, 1H), 4.35-4.15 (m, 2H), 3.79 (s, 3H), 1.24 (s, 3H).

### Example 232

### 1-[(RS)-6-methyl-2-(naphthalen-2-yl)-3-(pyridin-4-yl)-6,7-dihydropyrazolo[1,5-a]pyrazin-5(4H)-yl]prop-2-en-1-one

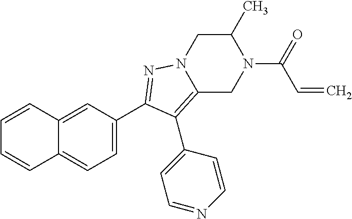 [SMILES: C=CC(=O)N1Cc2c(-c3ccncc3)c(-c3ccc4ccccc4c3)nn2CC1C]

To a solution of (RS)-6-methyl-2-(naphthalen-2-yl)-3-(pyridin-4-yl)-4,5,6,7-tetrahydropyrazolo[1,5-a]pyrazine hydrogen chloride (1/1) (80.0 mg, 92% purity, 195 μmol, Intermediate 400) and prop-2-enoyl chloride (26.5 mg, 293 μmol) in dichloromethane (2.0 ml) was added triethylamine (82 μl, 590 μmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give (RS)-1-(6-methyl-2-(naphthalen-2-yl)-3-(pyridin-4-yl)-6,7-dihydropyrazolo[1,5-a]pyrazin-5(4H)-yl) prop-2-en-1-one (21.5 mg, 98% purity, 27% yield) as a colourless oil.

LC-MS (Method 20): Rₜ=0.914 min; MS (ESIpos): m/z=395.1 [M+H]⁺.

¹H NMR (400 MHz, DMSO-d6), δ [ppm]=8.54 (d, J=6.0 Hz, 2H), 7.96 (s, 1H), 7.93-7.87 (m, 2H), 7.86-7.82 (m, 1H), 7.55-7.49 (m, 2H), 7.47 (dd, J=8.4, 1.2 Hz, 1H), 7.27 (d, J=6.0 Hz, 2H), 7.05-6.80 (m, 1H), 6.19 (dd, J=16.8, 2.0 Hz, 1H) 5.78 (d, J=11.6 Hz, 1H), 5.2 (d, J=17.6 Hz, 1H), 5.1-4.74 (m, 1H), 4.73-4.43 (m, 1H), 4.42-4.27 (m, 2H), 1.27 (S, 3H).

### Example 233

### 1-[(RS)-2-(2-chloro-1-benzothiophen-6-yl)-6-methyl-3-(pyridin-4-yl)-6,7-dihydropyrazolo[1,5-a]pyrazin-5(4H)-yl]prop-2-en-1-one

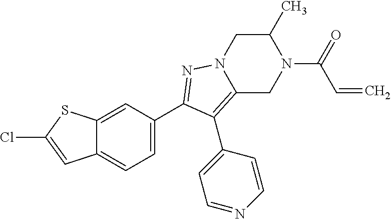 [SMILES: C=CC(=O)N1Cc2c(-c3ccncc3)c(-c3ccc4cc(Cl)sc4c3)nn2CC1C]

To a solution of (RS)-2-(2-chloro-1-benzothiophen-6-yl)-6-methyl-3-(pyridin-4-yl)-4,5,6,7-tetrahydropyrazolo[1,5-a]pyrazine hydrogen chloride (1/1) (110 mg, 264 μmol, Intermediate 404) in dichloromethane (5.0 ml) were added prop-2-enoyl chloride (26.2 mg, 290 μmol) and triethylamine (110 μl, 790 μmol) at 25° C. The mixture was stirred at 25° C. for 1 hour. The reaction mixture was concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-10 min 37-67% B; flow 25 ml/min; temperature: room temperature; Detector: UV 220/254 nm) to give 1-[(RS)-2-(2-chloro-1-benzothiophen-6-yl)-6-methyl-3-(pyridin-4-yl)-6,7-dihydropyrazolo[1,5-a]pyrazin-5(4H)-yl]prop-2-en-1-one (4.50 mg, 97% purity, 4% yield) as a white solid.

LC-MS (Method 14): Rₜ=0.798 minute; MS (ESIpos): m/z=435.1 [M+H]⁺.

¹H-NMR (400 MHz, CDCl₃), δ [ppm]=8.53 (d, J=6.0 Hz, 1H), 7.98 (s, 1H), 7.73 (d, J=8.0 Hz, 1H), 7.53 (s, 1H), 7.32 (dd, J=8.0, 1.2 Hz, 2H), 7.23 (d, J=6.0 Hz, 2H), 7.01-6.81 (m, 1H), 6.18 (dd, J=16.8, 2.8 Hz, 1H), 5.81-5.71 (m, 1H), 5.17 (d, J=17.6 Hz, 1H), 5.11-4.75 (m, 1H), 4.74-4.39 (m, 1H), 4.38-4.20 (m, 2H), 1.29-1.20 (m, 3H).

### Example 234

### 1-[(6RS)-2-(1-benzothiophen-6-yl)-6-methyl-3-(pyridin-4-yl)-6,7-dihydropyrazolo[1,5-a]pyrazin-5(4H)-yl]prop-2-en-1-one

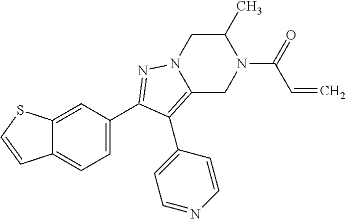 [SMILES: C=CC(=O)N1Cc2c(-c3ccncc3)c(-c3ccc4ccsc4c3)nn2CC1C]

To a solution of (RS)-2-(1-benzothiophen-6-yl)-6-methyl-3-(pyridin-4-yl)-4,5,6,7-tetrahydropyrazolo[1,5-a]pyrazine hydrogen chloride (1/1) (80.0 mg, 81% purity, 169 μmol, Intermediate 407) in dichloromethane (2.0 ml) were added prop-2-enoyl chloride (16.8 mg, 186 μmol) and triethylamine (71 μl, 510 μmol). The mixture was stirred at 25° C. for 30 minutes. The reaction mixture was concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give 1-[(6RS)-2-(1-benzothiophen-6-yl)-6-methyl-3-(pyridin-4-yl)-6,7-dihydropyrazolo[1,5-a]pyrazin-5(4H)-yl]prop-2-en-1-one (2.30 mg, 97% purity, 3% yield) as a white solid.

LC-MS (Method 16): Rₜ=1.098 min; MS (ESIpos): m/z=401.2 [M+H]⁺.

¹H NMR (400 MHz, DMSO-d6), δ [ppm]=8.53 (d, J=6.4 Hz, 2H), 8.03 (s, 1H), 7.84 (d, J=8.4 Hz, 1H), 7.79 (d, J=10.4 Hz, 1H), 7.49-7.41 (m, 1H), 7.36-7.29 (m, 1H), 7.27-7.20 (m, 2H), 7.04-6.80 (m, 1H), 6.25-6.05 (m, 1H), 5.84-5.70 (m, 1H), 5.25-5.11 (m, 1H), 5.10-4.69 (m, 1H), 4.68-4.38 (m, 1H), 4.37-4.14 (m, 2H), 1.36-1.12 (m, 3H).

### Example 235

### (2E)-1-[(RS)-2-(4-chloro-2-fluorophenyl)-6-methyl-3-(pyridin-4-yl)-6,7-dihydropyrazolo[1,5-a]pyrazin-5(4H)-yl]-4-(dimethylamino)but-2-en-1-one

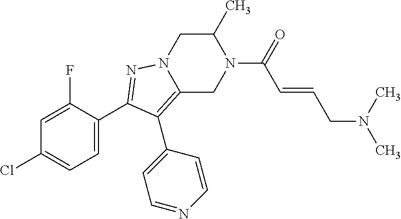 [SMILES: CC1Cn2nc(-c3ccc(Cl)cc3F)c(-c3ccncc3)c2CN1C(=O)/C=C/CN(C)C]

To a solution of (RS)-2-(4-chloro-2-fluorophenyl)-6-methyl-3-(pyridin-4-yl)-4,5,6,7-tetrahydropyrazolo[1,5-a]pyrazine hydrogen chloride (1/1) (130 mg, 70% purity, 240 μmol, Intermediate 385) and (2E)-4-(dimethylamino)but-2-enoic acid (34.1 mg, 264 μmol) in N,N-dimethylformamide added O-(7-azabenzotriazol-1-yl)-N,N,N′,N′-(2.2 ml) were tetramethyluronium hexafluorophosphate (137 mg, 360 μmol) and N,N-diisopropylethylamine (170 μl, 960 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give (2E)-1-[(RS)-2-(4-chloro-2-fluorophenyl)-6-methyl-3-(pyridin-4-yl)-6,7-dihydropyrazolo[1,5-a]pyrazin-5(4H)-yl]-4-(dimethylamino)but-2-en-1-one (36.1 mg, 95% purity, 31% yield) as a colourless oil.

LC-MS (Method 20): Rₜ=0.914 min; MS (ESIpos): m/z=395.1 [M+H]⁺

¹H NMR (400 MHz, DMSO-d6), δ [ppm]=8.51 (d, J=6.0 Hz, 2H), 7.56-7.50 (m, 1H), 7.46 (dd, J=10.0, 2.0 Hz, 1H), 7.38 (dd, J=8.4, 2.0 Hz, 1H), 7.14 (d, J=6.0 Hz, 2H), 6.67 (d, J=5.6 Hz, 2H), 5.21 (d, J=18.0 Hz, 1H), 5.04-4.76 (m, 1H), 4.71-4.40 (m, 1H), 4.39-4.17 (m, 2H), 3.04 (s, 2H), 2.14 (s, 6H), 1.23 (s, 3H).

### Example 236

### (2E)-1-[(RS)-2-(1-benzothiophen-6-yl)-6-methyl-3-(pyridin-4-yl)-6,7-dihydropyrazolo[1,5-a]pyrazin-5(4H)-yl]-4-(dimethylamino)but-2-en-1-one

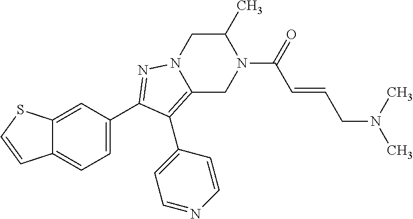 [SMILES: CC1Cn2nc(-c3ccc4ccsc4c3)c(-c3ccncc3)c2CN1C(=O)/C=C/CN(C)C]

To a solution of rac-(RS)-2-(1-benzothiophen-6-yl)-6-methyl-3-(pyridin-4-yl)-4,5,6,7-tetrahydropyrazolo[1,5-a]pyrazine hydrogen chloride (1/1) (100 mg, 97% purity, 253 μmol, Intermediate 407) in N,N-dimethylformamide (2.0 ml) were added (2E)-4-(dimethylamino)but-2-enoic acid (36.0 mg, 279 μmol), O-(7-azabenzotriazol-1-yl)-N,N,N′,N′-tetramethyluronium hexafluorophosphate (144 mg, 380 μmol) and N,N-diisopropylethylamine (130 μl, 760 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex C18 75*30 mm*3 μm; eluent A: 0.1% formic acid in water, eluent B: acetonitrile; gradient: 0-9 min 0-30% B; flow 25 ml/min; temperature: room temperature; Detector: UV 220/254 nm) to give (2E)-1-[(RS)-2-(1-benzothiophen-6-yl)-6-methyl-3-(pyridin-4-yl)-6,7-dihydropyrazolo[1,5-a]pyrazin-5(4H)-yl]-4-(dimethylamino)but-2-en-1-one (7.70 mg, 96% purity, 6% yield) as a white solid.

LC-MS (Method 16): Rₜ=0.582 min; MS (ESIpos): m/z=458.3 [M+H]⁺.

¹H NMR (400 MHz, DMSO-d6), δ [ppm]=8.53 (d, J=6.0 Hz, 2H), 8.03 (s, 1H), 7.83 (d, J=8.0 Hz, 1H), 7.78 (d, J=5.6 Hz, 1H), 7.47-7.46 (m, 1H), 7.36-7.29 (m, 1H), 7.27-7.20 (m, 2H), 6.83-6.52 (m, 2H), 5.27-5.08 (m, 1H), 5.07-4.70 (m, 1H), 4.69-4.38 (m, 1H), 4.37-4.18 (m, 2H), 3.25-3.17 (m, 2H), 2.27 (s, 6H), 1.30-1.22 (m, 3H).

### Example 237

### 1-[(RS)-2-(4-chloro-2-fluorophenyl)-6-methyl-3-(pyridin-4-yl)-6,7-dihydropyrazolo[1,5-a]pyrazin-5(4H)-yl]-2-fluoroprop-2-en-1-one

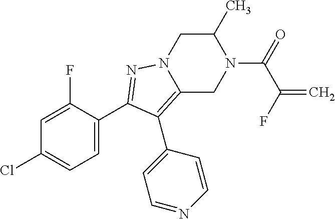 [SMILES: C=C(F)C(=O)N1Cc2c(-c3ccncc3)c(-c3ccc(Cl)cc3F)nn2CC1C]

To a solution of (RS)-2-(4-chloro-2-fluorophenyl)-6-methyl-3-(pyridin-4-yl)-4,5,6,7-tetrahydropyrazolo[1,5-a]pyrazine hydrogen chloride (1/1) (70.0 mg, 98% purity, 181 μmol, Intermediate 385) and 2-fluoroprop-2-enoic acid (17.9 mg, 199 μmol) in N,N-dimethylformamide (2.0 ml) were added O-(7-azabenzotriazol-1-yl)-N,N,N′,N′-tetramethyluronium hexafluorophosphate (103 mg, 271 μmol) and N,N-diisopropylethylamine (130 μl, 720 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Waters Xbridge 150*25 mm*5 μm; eluent A: 0.1% ammonia hydroxide in water, eluent B: acetonitrile; gradient: 0-9 min 30-60% B; flow 25 ml/min; temperature: room temperature; Detector: UV 220/254 nm) to give (RS)-1-(2-(4-chloro-2-fluorophenyl)-6-methyl-3-(pyridin-4-yl)-6,7-dihydropyrazolo[1,5-a]pyrazin-5(4H)-yl)-2-fluoroprop-2-en-1-one (33.5 mg, 99% purity, 44% yield) as a colourless oil.

LC-MS (Method 20): Rₜ=0.939 min; MS (ESIpos): m/z=415.1 [M+H]⁺.

¹H NMR (400 MHz, DMSO-d₆), δ [ppm]=8.51 (d, J=6.0 Hz, 2H), 7.56-7.49 (m, 1H), 7.46 (dd, J=10.0, 2.0 Hz, 1H), 7.39 (dd, J=8.0, 2.0 Hz, 1H), 7.14 (d, J=6.0 Hz, 2H), 5.4 (s, 1H), 5.38-5.26 (m, 1H), 5.12 (d, J=17.6 Hz, 1H), 4.96-4.76 (m, 2H), 4.43-4.34 (m, 1H), 4.31-4.25 (m, 1H), 1.32 (d, J=6.8 Hz, 3H).

### Example 238

### 1-[(RS)-2-(1-benzothiophen-6-yl)-6-methyl-3-(pyridin-4-yl)-6,7-dihydropyrazolo[1,5-a]pyrazin-5(4H)-yl]-2-fluoroprop-2-en-1-one

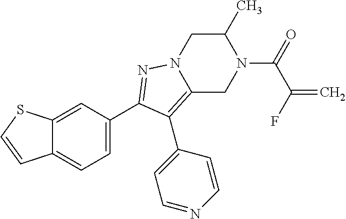 [SMILES: C=C(F)C(=O)N1Cc2c(-c3ccncc3)c(-c3ccc4ccsc4c3)nn2CC1C]

To a solution of (RS)-2-(1-benzothiophen-6-yl)-6-methyl-3-(pyridin-4-yl)-4,5,6,7-tetrahydropyrazolo[1,5-a]pyrazine (100 mg, 97% purity, 280 μmol, Intermediate 407) in N,N-dimethylformamide (2.0 ml) were added 2-fluoroprop-2-enoic acid (37.8 mg, 420 μmol), O-(7-azabenzotriazol-1-yl)-N,N,N′,N′-tetramethyluronium hexafluorophosphate (160 mg, 420 μmol) and N,N-diisopropylethylamine (150 μl, 840 μmol) at 25° C. The mixture was stirred at 25° C. for 16 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex C18 75*30 mm*3 μm; eluent A: 0.1% formic acid in water, eluent B: acetonitrile; gradient: 0-9 min 14-44% B; flow 25 ml/min; temperature: room temperature; Detector: UV 220/254 nm) to give 1-[(RS)-2-(1-benzothiophen-6-yl)-6-methyl-3-(pyridin-4-yl)-6,7-dihydropyrazolo[1,5-a]pyrazin-5(4H)-yl]-2-fluoroprop-2-en-1-one (51.4 mg, 95% purity, 42% yield) as a white solid.

LC-MS (Method 16): Rₜ=0.615 min; MS (ESIpos): m/z=419.3 [M+H]⁺.

¹H NMR (400 MHz, DMSO-d₆), δ [ppm]=8.67-8.45 (m, 2H), 8.03 (s, 1H), 7.85 (d, J=8.0 Hz, 1H), 7.80 (d, J=8.0 Hz, 1H), 7.49-7.44 (m, 1H), 7.41-7.36 (m, 1H), 7.36-7.31 (m, 1H), 5.41 (s, 1H), 5.39-5.26 (m, 1H), 5.17-5.02 (m, 1H), 4.87-4.69 (m, 1H), 4.45-4.35 (m, 1H), 4.33-4.25 (m, 1H), 1.34 (d, J=6.8 Hz, 1H).

### Example 239

### 1-[(RS)-2-(4-fluorophenyl)-4-methyl-3-(pyridin-4-yl)-6,7-dihydropyrazolo[1,5-a]pyrazin-5(4H)-yl]prop-2-en-1-one

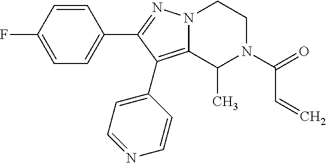 [SMILES: C=CC(=O)N1CCn2nc(-c3ccc(F)cc3)c(-c3ccncc3)c2C1C]

To a solution of (RS)-2-(4-fluorophenyl)-4-methyl-3-(pyridin-4-yl)-4,5,6,7-tetrahydropyrazolo[1,5-a]pyrazine hydrogen chloride (1/1) (118 mg, 0.342 mmol, Intermediate 418) in dichloromethane (5 ml) were added prop-2-enoyl chloride (34.1 mg, 0.376 mmol) and trimethylamine (0.14 ml, 1.00 mmol) at 25° C. The mixture was stirred at 25° C. for 2 hours. The reaction mixture was concentrated in vacuo to give a residue and the residue was purified by preparative HPLC (Instrument: Gilson-281; Column: Phenomenex Luna C18 150*25 mm*10 μm; eluent A: 0.225% formic acid in water, eluent B: acetonitrile; gradient: 0-10 min 1-41% B; flow 25 ml/min; temperature: room temperature; Detector: UV 220/254 nm) to give 1-[(RS)-2-(4-fluorophenyl)-4-methyl-3-(pyridin-4-yl)-6,7-dihydropyrazolo[1,5-a]pyrazin-5(4H)-yl]prop-2-en-1-one (47.2 mg, 95% purity, 36% yield) as a white solid.

LC-MS (Method 14): Rₜ=0.681 minute; MS (ESIpos): m/z=363.1 [M+H]⁺.

¹H-NMR (400 MHz, DMSO), δ [ppm]=8.59 (s, 2H), 7.37-7.20 (m, 4H), 7.15 (t, J=7.2 Hz, 2H), 7.05-6.87 (m, 1H), 6.30-6.14 (m, 1H), 5.91-5.57 (m, 2H), 4.87-4.37 (m, 1H), 4.35-4.27 (m, 1H), 4.26-4.01 (m, 1H), 3.87-3.51 (m, 1H), 1.18-1.03 (m, 3H).

### Example 240

### 1-[(RS)-2-(4-chloro-2-fluorophenyl)-4-methyl-3-(pyridin-4-yl)-6,7-dihydropyrazolo[1,5-a]pyrazin-5(4H)-yl]prop-2-en-1-one

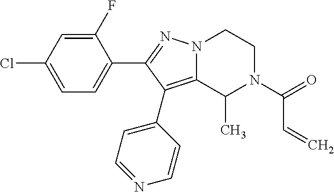 [SMILES: C=CC(=O)N1CCn2nc(-c3ccc(Cl)cc3F)c(-c3ccncc3)c2C1C]

To a solution of (RS)-2-(4-chloro-2-fluorophenyl)-4-methyl-3-(pyridin-4-yl)-4,5,6,7-tetrahydropyrazolo[1,5-a]pyrazine hydrogen chloride (1/1) (85.0 mg, 0.224 mmol, Intermediate 422) in dichloromethane (2 ml) were added prop-2-enoyl chloride (24.3 mg, 0.269 mmol) and trimethylamine (0.1 ml, 0.670 mmol) at 25° C. The reaction mixture was stirred at 25° C. for 2 hours. The reaction mixture was diluted with water and extracted with ethyl acetate. The combined organic phase was washed with brine, dried over anhydrous sodium sulfate, filtered and concentrated in vacuo to give a residue. The residue was purified by preparative HPLC (Instrument: Gilson-281; Column: Phenomenex Luna C18 150*25 mm*10 μm; eluent A: 0.1% ammonium hydroxide in water, eluent B: acetonitrile; gradient: 0-10 min 1-41% B; flow 25 ml/min; temperature: room temperature; Detector: UV 220/254 nm) to give 1-[(RS)-2-(4-chloro-2-fluorophenyl)-4-methyl-3-(pyridin-4-yl)-6,7-dihydropyrazolo[1,5-a]pyrazin-5(4H)-yl]prop-2-en-1-one (23.5 mg, 99% purity, 26% yield) as a white solid.

LC-MS (Method 14): Rₜ=0.880 minute; MS (ESIpos): m/z=397.2 [M+H]⁺.

¹H-NMR (400 MHz, DMSO), δ [ppm]=8.69-8.37 (m, 2H), 7.47 (t, J=8.4 Hz, 1H), 7.41 (dd, J=10.0, 1.6 Hz, 1H), 7.35 (dd, J=8.4, 1.6 Hz, 1H), 7.28-7.11 (m, 2H), 7.10-6.88 (m, 1H), 6.31-6.16 (m, 1H), 6.08-5.72 (m, 2H), 4.87-4.40 (m, 1H), 4.37-4.30 (m, 1H), 4.29-4.05 (m, 1H), 3.89-3.39 (m, 1H), 1.20-1.07 (m, 3H).

### Example 241

### 1-[(RS)-2-(4-methoxyphenyl)-4-methyl-3-(pyridin-4-yl)-6,7-dihydropyrazolo[1,5-a]pyrazin-5(4H)-yl]prop-2-en-1-one

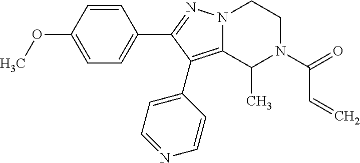 [SMILES: C=CC(=O)N1CCn2nc(-c3ccc(OC)cc3)c(-c3ccncc3)c2C1C]

To a solution of (RS)-2-(4-methoxyphenyl)-4-methyl-3-(pyridin-4-yl)-4,5,6,7-tetrahydropyrazolo[1,5-a]pyrazine hydrogen chloride (1/1) (178 mg, 0.499 mmol, Intermediate 424) in dichloromethane (10 ml) were added prop-2-enoyl chloride (49.7 mg, 0.549 mmol) and triethylamine (0.210 ml, 1.5 mmol). The mixture was stirred at 25° C. for 1 hour. The reaction mixture was concentrated in vacuo to give a residue and the residue was purified by preparative HPLC (Instrument: Gilson-281; Column: Phenomenex Luna C18 150*25 mm*10 μm; eluent A: 0.225% formic acid in water, eluent B: acetonitrile; gradient: 0-10 min 10-40% B; flow 25 ml/min; temperature: room temperature; Detector: UV 220/254 nm) to give 1-[(RS)-2-(4-methoxyphenyl)-4-methyl-3-(pyridin-4-yl)-6,7-dihydropyrazolo[1,5-a]pyrazin-5(4H)-yl]prop-2-en-1-one (80.2 mg, 94% purity, 40% yield) as a white solid.

LC-MS (Method 14): Rₜ=0.849 minute; MS (ESIpos): m/z=375.2 [M+H]⁺.

1H-NMR (400 MHz, DMSO), δ [ppm]=8.59 (s, 2H), 7.44-7.17 (m, 4H), 7.07-6.79 (m, 3H), 6.32-6.13 (m, 1H), 5.96-5.54 (m, 2H), 4.86-4.39 (m, 1H), 4.34-4.25 (m, 1H), 4.24-3.98 (m, 1H), 3.89-3.77 (m, 1H), 3.74 (s, 3H), 1.30-0.97 (m, 3H).

### Example 242

### 1-[(RS)-2-(1-benzothiophen-5-yl)-4-methyl-3-(pyridin-4-yl)-6,7-dihydropyrazolo[1,5-a]pyrazin-5(4H)-yl]prop-2-en-1-one

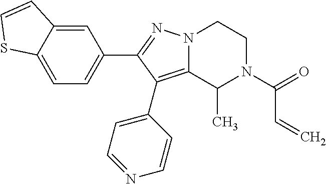 [SMILES: C=CC(=O)N1CCn2nc(-c3ccc4sccc4c3)c(-c3ccncc3)c2C1C]

To a solution of (RS)-2-(1-benzothiophen-5-yl)-4-methyl-3-(pyridin-4-yl)-4,5,6,7-tetrahydropyrazolo[1,5-a]pyrazine hydrogen chloride (1/1) (130 mg, 84% purity, 0.285 mmol, Intermediate 426) in dichloromethane (2 ml) were added prop-2-enoyl chloride (28.4 mg, 0.314 mmol) and trimethylamine (0.120 ml, 0.860 mmol) at 25° C. The reaction mixture was stirred at 25° C. for 30 minutes. The reaction mixture was poured into water and extracted with ethyl acetate. The combined organic phase was washed with brine, dried over anhydrous sodium sulfate, filtered and concentrated in vacuo to give a residue. The residue was purified by preparative HPLC (Instrument: Gilson-281; Column: Waters Xbridge 150*25 mm*5 μm; eluent A: 0.1% ammonia hydroxide in water, eluent B: acetonitrile; gradient: 0-9 min 30-58% B; flow 25 ml/min; temperature: room temperature; Detector: UV 220/254 nm) to give 1-[(RS)-2-(1-benzothiophen-5-yl)-4-methyl-3-(pyridin-4-yl)-6,7-dihydropyrazolo[1,5-a]pyrazin-5(4H)-yl]prop-2-en-1-one (11.0 mg, 95% purity, 9% yield) as a white solid.

LC-MS (Method 14): Rₜ=0.910 minute; MS (ESIpos): m/z=401.1 [M+H]⁺.

¹H-NMR (400 MHz, DMSO), δ [ppm]=8.67-8.50 (m, 2H), 7.92 (d, J=8.4 Hz, 1H), 7.84 (d, J=1.2 Hz, 1H), 7.76 (d, J=5.6 Hz, 1H), 7.39 (d, J=5.6 Hz, 2H), 7.33-7.20 (m, 2H), 7.07-6.90 (m, 1H), 6.31-6.16 (m, 1H), 6.00-5.64 (m, 2H), 4.92-4.39 (m, 1H), 4.38-4.30 (m, 1H), 4.29-4.09 (m, 1H), 3.92-3.75 (m, 1H), 1.20-1.08 (m, 4H)

### Example 243

### 1-[(RS)-2-(1H-indol-6-yl)-4-methyl-3-(pyridin-4-yl)-6,7-dihydropyrazolo[1,5-a]pyrazin-5(4H)-yl]prop-2-en-1-one

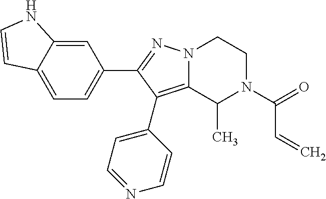 [SMILES: C=CC(=O)N1CCn2nc(-c3ccc4cc[nH]c4c3)c(-c3ccncc3)c2C1C]

To a solution of (RS)-2-(1H-indol-6-yl)-4-methyl-3-(pyridin-4-yl)-4,5,6,7-tetrahydropyrazolo[1,5-a]pyrazine hydrogen chloride (1/1) (160 mg, 0.437 mmol, Intermediate 428) in dichloromethane (10 ml) were added prop-2-enoyl chloride (47.5 mg, 0.525 mmol) and triethylamine (0.180 ml, 1.3 mmol) at 0° C. The mixture was stirred at 25° C. for 1 hour. The reaction mixture was diluted with water and extracted with ethyl acetate. The combined organic phase was washed with brine, dried over anhydrous sodium sulfate, filtered and concentrated in vacuo to give a residue. The residue was purified by preparative HPLC (Instrument: Gilson-281; Column: Waters Xbridge C18 150*25 mm*5 μm; eluent A: 0.1% ammonium hydroxide in water, eluent B: acetonitrile; gradient: 0-9 min 24-65% B; flow 25 ml/min; temperature: room temperature; Detector: UV 220/254 nm) to give 1-[(RS)-2-(1H-indol-6-yl)-4-methyl-3-(pyridin-4-yl)-6,7-dihydropyrazolo[1,5-a]pyrazin-5(4H)-yl]prop-2-en-1-one (34.4 mg, 95% purity, 19% yield) as a white solid.

LC-MS (Method 14): Rₜ=0.755 minute; MS (ESIpos): m/z=384.1 [M+H]⁺.

¹H-NMR (400 MHz, DMSO), δ [ppm]=11.06 (s, 1H), 8.57 (s, 2H), 7.44 (d, J=8.4 Hz, 1H), 7.31 (s, 4H), 7.08-6.89 (m, 2H), 6.38 (s, 1H), 6.29-6.15 (m, 1H), 5.94-5.62 (m, 2H), 4.87-4.39 (m, 1H), 4.37-4.28 (m, 1H), 4.27-4.01 (m, 1H), 3.91-3.38 (m, 1H), 1.18-1.07 (m, 3H).

### Example 244

### (2E)-1-[(RS)-2-(4-chloro-2-fluorophenyl)-4-methyl-3-(pyridin-4-yl)-6,7-dihydropyrazolo[1,5-a]pyrazin-5(4H)-yl]-4-(dimethylamino)but-2-en-1-one

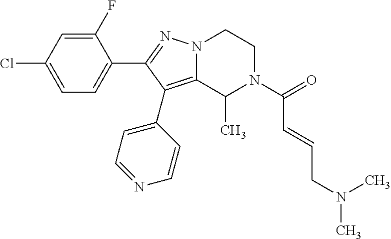 [SMILES: CC1c2c(-c3ccncc3)c(-c3ccc(Cl)cc3F)nn2CCN1C(=O)/C=C/CN(C)C]

To a solution of (RS)-2-(4-chloro-2-fluorophenyl)-4-methyl-3-(pyridin-4-yl)-4,5,6,7-tetrahydropyrazolo[1,5-a]pyrazine hydrogen chloride (1/1) (80.0 mg, 0.211 mmol, Intermediate 422), (2E)-4-(dimethylamino)but-2-enoic acid (35.4 mg, 0.274 mmol) in N,N-dimethylformamide (2 ml) were added (1-[bis(dimethylamino)methylene]-1H-1,2,3-triazolo[4,5-b]pyridinium 3-oxid hexafluorophosphate (120 mg, 0.316 mmol) and N,N-diisopropylethylamine (0.110 ml, 0.630 mmol) at 20° C. After stirring at 20° C. for 2 hours, the reaction mixture was diluted with water and extracted with ethyl acetate. The combined organic phase was washed with brine, dried over anhydrous sodium sulfate, filtered and concentrated in vacuo to give a residue. The residue was purified by preparative HPLC (Instrument: Gilson-281; Column: Waters Xbridge C18 150*25 mm*5 μm; eluent A: 0.1% ammonium hydroxide in water, eluent B: acetonitrile; gradient: 0-9 min 34-64% B; flow 25 ml/min; temperature: room temperature; Detector: UV 220/254 nm) to give (2E)-1-[(RS)-2-(4-chloro-2-fluorophenyl)-4-methyl-3-(pyridin-4-yl)-6,7-dihydropyrazolo[1,5-a]pyrazin-5(4H)-yl]-4-(dimethylamino)but-2-en-1-one (34.2 mg, 95% purity, 34% yield) as a white solid.

LC-MS (Method 16): Rₜ=0.909 minute; MS (ESIpos): m/z=454.3 [M+H]⁺.

¹H-NMR (400 MHz, DMSO), δ [ppm]=8.53 (s, 2H), 7.47 (t, J=8.4 Hz, 1H), 7.41 (dd, J=10.0, 1.6 Hz, 1H), 7.34 (dd, J=8.4, 2.0 Hz, 1H), 7.28-7.08 (m, 2H), 6.84-6.66 (m, 2H), 6.09-5.66 (m, 1H), 4.87-4.37 (m, 1H), 4.37-4.18 (m, 2H), 4.15-3.73 (m, 1H), 3.15-3.02 (m, 2H), 2.17 (s, 6H), 1.21-1.08 (m, 3H).

### Example 245

### (2E)-1-[(6S)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one

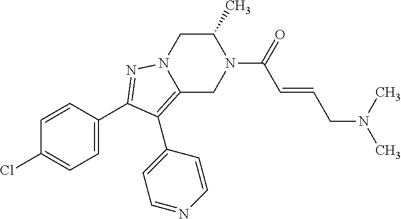 [SMILES: C[C@H]1Cn2nc(-c3ccc(Cl)cc3)c(-c3ccncc3)c2CN1C(=O)/C=C/CN(C)C]

To a solution of (6S)-2-(4-chiorophenyl)-6-methyl-3-(pyridin-4-yl)-4,5,6,7-tetrahydropyrazolo[1,5-a]pyrazine hydrogen chloride (1/1) (2.80 g, 7.75 mmol, Intermediate 432) and (2E)-4-(dimethylamino)but-2-enoic acid hydrogen chloride (1/1) (1.93 g, 11.6 mmol) in dichloromethane (40 ml) were added N,N-diisopropylethylamine (5.4 ml) and O-(7-azabenzotriazol-1-yl)-N,N,N,N-tetramethyl uronium hexafluorophosphate (4.42 g, 11.6 mmol) at 20° C. and the mixture was stirred at 20° C. for 16 hours. The reaction solution was washed with water and extracted with dichloromethane. The combined organic layers were washed with brine, dried over anhydrous sodium sulfate, filtered and concentrated in vacuo to give a residue. The residue was purified by preparative HPLC [Instrument: CASWH-PREP-HPLC-G; Column: Kromasil Eternity XT 250*80 mm*10 μm, eluent A: water (0.2% ammonia hydroxide), eluent B: acetonitrile; gradient: 0-20 min 31%-61% B; flow 140 ml/min; temperature: RT; Detector: UV 220/254 nm] to give (2E)-1-[(6S)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one (1.87 g, 4.17 mmol, 97% purity, 54% yield) as a brown solid.

Optical rotation: [α]_{D}=−92.919° (c=0.287 g/100 ml, ACN)

LC-MS (Method 16): Rₜ=0.601 min; MS (ESIpos): m/z=436.1 [M+H]⁺.

¹H NMR (400 MHz, DMSO-d6), δ [ppm]=8.56 (d, J=5.6 Hz, 2H), 7.45-7.34 (m, 4H), 7.24 (d, J=5.6 Hz, 2H), 6.80-6.56 (m, 2H), 5.19-5.03 (m, 1H), 5.00-4.82 (m, 1H), 4.50-4.27 (m, 1H), 4.26-4.18 (m, 1H), 3.30 (s, 1H), 3.04 (br s, 2H), 2.14 (br s, 6H), 1.23 (br s, 3H).

### Example 246

### (2E)-1-[(6R)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one

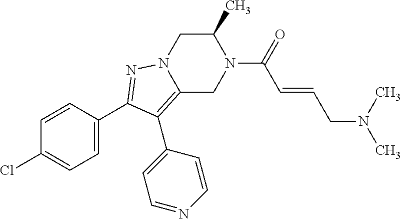 [SMILES: C[C@@H]1Cn2nc(-c3ccc(Cl)cc3)c(-c3ccncc3)c2CN1C(=O)/C=C/CN(C)C]

To a solution of (6R)-2-(4-chlorophenyl)-6-methyl-3-(pyridin-4-yl)-4,5,6,7-tetrahydropyrazolo[1,5-a]pyrazine hydrogen chloride (1/1) (3.50 g, 9.69 mmol, Intermediate 436) and (2E)-4-(dimethylamino)but-2-enoic acid hydrogen chloride (1/1) (2.41 g, 14.5 mmol) in dichloromethane (60 ml) were added N,N-diisopropylethylamine (7 ml, 39.0 mmol) and O-(7-azabenzotriazol-1-yl)-N,N,N,N-tetramethyl uronium hexafluorophosphate (5.53 g, 14.5 mmol) at 20° C. and the reaction mixture was stirred at 20° C. for 16 hours. The reaction solution was washed with water and extracted with dichloromethane. The combined organic layers were washed with brine, dried over anhydrous sodium sulfate, filtered and concentrated in vacuo to give a residue. The residue was purified by preparative HPLC [Instrument: CASWH-PREP-HPLC-D; Column: Phenomenex luna C18 (250*70 mm, 10 μm), eluent A: water (0.2% formic acid), eluent B: acetonitrile; gradient: 0-10 min 15%-45% B; flow 140 ml/min; temperature: RT; Detector: UV 220/254 nm] to give (2E)-1-[(6R)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one (1.75 g, 3.95 mmol, 99% purity, 41% yield) as a brown solid.

Optical rotation: [α]_{D}=+117.392° (c=0.38 g/100 ml, ACN) LC-MS (Method 16): Rₜ=0.559 min; MS (ESIpos): m/z=436.3 [M+H]⁺.

¹H NMR (400 MHz, DMSO-d6), δ [ppm]=8.56 (d, J=5.6 Hz, 2H), 7.46-7.33 (m, 4H), 7.24 (d, J=5.6 Hz, 2H), 6.81-6.56 (m, 2H), 5.19-5.02 (m, 1H), 5.00-4.74 (m, 1H), 4.59-4.39 (m, 1H), 4.37 (br s, 2H), 3.07 (br s, 2H), 2.16 (br s, 6H), 1.23 (br s, 3H).

### Example 247

### 1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one (isomer 2)

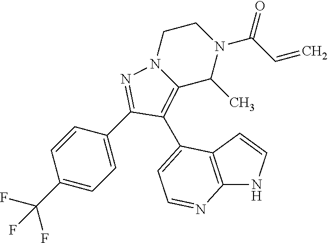 [SMILES: C=CC(=O)N1CCn2nc(-c3ccc(C(F)(F)F)cc3)c(-c3ccnc4[nH]ccc34)c2C1C]

To a solution of 4-methyl-3-(1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2) (75.0 mg, 0.173 mmol, Intermediate 448) in dichloromethane (2.0 ml) were added prop-2-enoyl chloride (15.6 mg, 0.173 mmol) and trimethylamine (0.048 ml, 0.350 mmol) at 0° C. The mixture was stirred at 0° C. for 1 hour. The reaction mixture was concentrated under reduced pressure to give a residue. The residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 45%-75% B; flow 25 ml/min; temperature: RT; Detector: UV 220/254 nm] to give 1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one (isomer 2) (10.8 mg, 0.0228 mmol, 95% purity, 13% yield) as a white solid.

LC-MS (Method 16): Rₜ=0.912 min; MS (ESIpos): m/z=452.1 [M+H]⁺.

¹H NMR (400 MHz, DMSO-d6), δ [ppm]=12.07-11.60 (m, 1H), 8.42-8.15 (m, 1H), 7.62-7.45 (m, 4H), 7.42-7.25 (m, 1H), 7.02-6.88 (m, 1H), 6.2-6.13 (m, 1H), 5.78 (d, J=10.4 Hz, 2H), 4.87-4.73 (m, 1H), 4.55-4.23 (m, 2H), 4.22-4.06 (m, 1H), 3.92-3.75 (m, 1H), 3.54-3.41 (m, 1H), 1.05-0.82 (m, 3H).

### Example 248

### 1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one

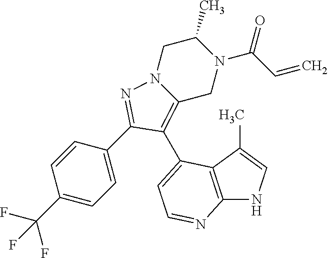 [SMILES: C=CC(=O)N1Cc2c(-c3ccnc4[nH]cc(C)c34)c(-c3ccc(C(F)(F)F)cc3)nn2C[C@@H]1C]

To a solution of (6S)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (160 mg, 0.357 mmol, Intermediate 452) in dichloromethane (5.0 ml) were added prop-2-enoyl chloride (32.3 mg, 0.357 mmol) and triethylamine (0.15 ml, 1.07 mmol) at 0° C. The mixture was stirred at 0° C. for 1 hour. The reaction mixture was concentrated under reduced pressure to give a residue. The residue was purified by preparative HPLC [Instrument: ACSWH-GX-P; Column: Phenomenex luna C18 150*25 mm*10 μm, eluent A: water (0.2% formic acid), eluent B: acetonitrile; gradient: 0-10 min 37%-64% B; flow 25 ml/min; temperature: RT; Detector: UV 220/254 nm] to give 1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one (25.1 mg, 0.0509 mmol, 94% purity, 14% yield) as a white solid.

LC-MS (Method 16): Rₜ=0.781 min; MS (ESIpos): m/z=466.1 [M+H]⁺.

¹H NMR (400 MHz, DMSO-d6), δ [ppm]=11.31-11.15 (m, 1H), 8.31-8.19 (m, 1H), 7.60-7.50 (m, 4H), 7.15 (s, 1H), 6.93 (t, J=4.8 Hz, 1H), 6.83-6.70 (m, 1H), 6.16-6.07 (m, 1H), 5.74-5.66 (m, 1H), 5.09-4.97 (m, 1H), 4.94-4.73 (m, 1H), 4.46-4.38 (m, 1H), 4.36-4.28 (m, 1H), 4.27-4.05 (m, 1H), 1.79-1.70 (m, 3H), 1.29-1.20 (m, 3H).

### Example 249

### 1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one (isomer 1)

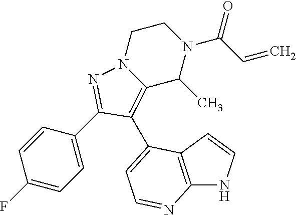 [SMILES: C=CC(=O)N1CCn2nc(-c3ccc(F)cc3)c(-c3ccnc4[nH]ccc34)c2C1C]

To a solution of 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 1) (80.0 mg, 0.208 mmol, Intermediate 457) and prop-2-enoyl chloride (22.6 mg, 0.250 mmol) in dichloromethane (3 ml) was added trimethylamine (0.058 ml, 0.416 mmol) at 0° C. The mixture was stirred at 0° C. for 1 hour. The reaction mixture was concentrated in vacuo to give a residue. The residue was purified by preparative-HPLC (columnPhenomenex luna C18 150*25 mm*10 μm; mobile phase: water (TFA)-ACN; B %: 24%-54%, 10 min) to give 1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one (isomer 1) (35.8 mg, 0.0838 mmol, 94% purity, 40% yield) as a white solid.

LC-MS (Method 16): Rₜ=0.697 min; MS (ESIpos): m/z=402.1 [M+H]⁺.

¹H NMR (400 MHz, DMSO-d6), δ [ppm]=11.50 (br s, 1H), 8.26 (d, J=4.8 Hz, 1H), 7.47-7.20 (m, 3H), 7.10-6.90 (m, 3H), 6.81 (dd, J=15.2, 10.4 Hz, 1H), 6.15 (dd, J=16.8, 2.4 Hz, 1H), 6.03 (br s, 1H), 5.74 (dd, J=10.8, 2.0 Hz, 1H), 5.68-5.55 (m, 1H), 4.64-4.45 (m, 1H), 4.36 (dd, J=12.8, 2.8 Hz, 1H), 4.21 (t, J=11.2, 4.0 Hz, 1H), 3.82-3.61 (m, 1H), 1.00 (d, J=6.8 Hz, 3H).

### Example 250

### 1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one (isomer 2)

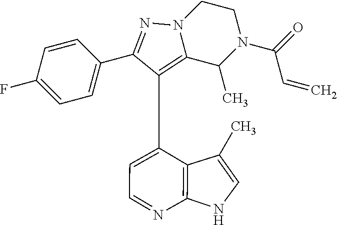 [SMILES: C=CC(=O)N1CCn2nc(-c3ccc(F)cc3)c(-c3ccnc4[nH]cc(C)c34)c2C1C]

To a solution of 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 2) (100 mg, 0.277 mmol, Intermediate 459) in dichloromethane (20 ml) were added prop-2-enoyl chloride (25.0 mg, 0.277 mmol) and trimethylamine (0.120 ml, 0.830 mmol) at −10° C. The mixture was stirred at −10° C. for 1 hour. The reaction mixture was concentrated under reduced pressure to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 14%-54% B; flow 25 ml/min; temperature: RT; Detector: UV 220/254 nm] to give the title compound (18.7 mg, 0.0420 mmol, 93% purity, 15% yield) as a white solid.

LC-MS (Method 16): Rₜ=0.812 min; MS (ESIpos): m/z=416.2 [M+H]⁺.

¹H NMR (400 MHz, DMSO-d6), 0 [ppm]=11.59-11.34 (d, J=3.6 Hz, 1H), 8.32-8.16 (m, 1H), 7.33 (d, J=5.2 Hz, 2H), 7.27-7.13 (m, 1H), 7.08-7.00 (m, 2H), 6.99-6.89 (m, 1H), 6.81 (d, J=4.4 Hz, 1H), 6.17 (d, J=16.4 Hz, 1H), 5.77 (d, J=10.8 Hz, 1H), 5.61-5.34 (m, 1H), 4.51-4.30 (m, 2H), 4.29-4.15 (m, 1H), 3.92-3.77 (m, 1H), 1.70 (d, J=11.6 Hz, 3H), 1.09-0.85 (m, 3H).

### Example 251

### 1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one

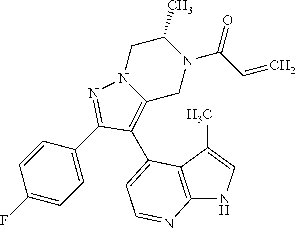 [SMILES: C=CC(=O)N1Cc2c(-c3ccnc4[nH]cc(C)c34)c(-c3ccc(F)cc3)nn2C[C@@H]1C]

To a solution of (6S)-2-(4-fluorophenyl)-6-methyl-3-(3-methyl-1H-pyrrolo[2,3-b]pyridin-4-yl)-4,5,6,7-tetrahydropyrazolo[1,5-a]pyrazine (75.0 mg, 0.208 mmol, Intermediate 463) in dichloromethane (15 ml) were added prop-2-enoyl chloride (18.8 mg, 208 μmol) and triethylamine (0.087 ml, 0.620 mmol) at −10° C. The mixture was stirred at −10° C. for 1 hour. The reaction mixture was concentrated under reduced pressure to give a residue. The residue was purified by preparative HPLC [Instrument: ACSWH-GX-E; Column: Phenomenex luna C18 150*25 mm*10 μm, eluent A: water (0.2% formic acid), eluent B: acetonitrile; gradient: 0-10 min 28%-58% B; flow 25 ml/min; temperature: RT; Detector: UV 220/254 nm] to give 1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one (10.0 mg, 0.0238 mmol, 99% purity, 11% yield) as a white solid.

LC-MS (Method 16): Rₜ=0.709 min; MS (ESIpos): m/z=416.1 [M+H]⁺.

¹H NMR (400 MHz, DMSO-d6), δ [ppm]=11.56-11.39 (m, 1H), 8.23 (dd, J=6.4, 4.8 Hz, 1H), 7.41-7.31 (m, 2H), 7.20-7.15 (m, 1H), 7.03 (d, J=8.8 Hz, 2H), 6.99-6.81 (m, 2H), 6.21-6.05 (m, 1H), 5.84-5.60 (m, 1H), 4.96-4.84 (m, 1H), 4.82-4.65 (m, 1H), 4.50-4.33 (m, 1H), 4.33-4.24 (m, 1H), 4.17-3.83 (m, 1H), 1.80-1.65 (m, 3H), 1.29-1.16 (m, 3H).

### Example 252

### 4-(dimethylamino)-1-[4-methyl-3-(1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one (isomer 2)

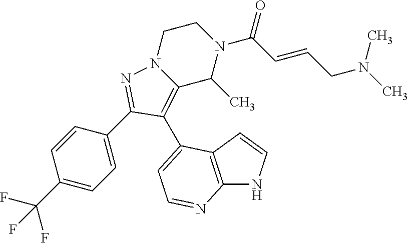 [SMILES: CC1c2c(-c3ccnc4[nH]ccc34)c(-c3ccc(C(F)(F)F)cc3)nn2CCN1C(=O)/C=C/CN(C)C]

To a solution of (2E)-4-(dimethylamino)but-2-enoic acid (44.7 mg, 0.346 mmol) in dichloromethane (2.0 ml) were added N,N-diisopropylethylamine (0.16 ml, 0.920 mmol) and O-(7-azabenzotriazol-1-yl)-N,N,N,N-tetramethyl uronium hexafluorophosphate (131 mg, 0.346 mmol) at 20° C. The mixture was stirred at 20° C. for 0.1 hour. To the mixture above was added 4-methyl-3-(1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2) (100 mg, 0.230 mmol, Intermediate 448) at 20° C. and the mixture was stirred at 20° C. for 2 hours. The reaction solution was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 14%-44% B; flow 25 ml/min; temperature: RT; Detector: UV 220/254 nm] to give (2E)-4-(dimethylamino)-1-[4-methyl-3-(1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one (isomer 2) (17.1 mg, 0.0325 mmol, 97% purity, 14% yield) as a white solid.

LC-MS (Method 16): Rₜ=0.662 min; MS (ESIpos): m/z=509.3 [M+H]⁺.

¹H NMR (400 MHz, DMSO-d6), δ [ppm]=11.61-11.44 (m, 1H), 8.27 (d, J=4.8 Hz, 1H), 7.52 (s, 4H), 7.38 (br s, 1H), 6.96 (br s, 1H), 6.66 (br s, 2H), 6.09-5.97 (m, 1H), 5.71-5.53 (m, 1H), 4.64-4.45 (m, 1H), 4.40 (dd, J=12.8, 2.8 Hz, 1H), 4.30-4.18 (m, 1H), 3.79-3.65 (m, 1H), 3.22 (br s, 2H), 2.30 (s, 6H), 1.01 (d, J=6.4 Hz, 3H).

### Example 253

### 4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one (isomer 2)

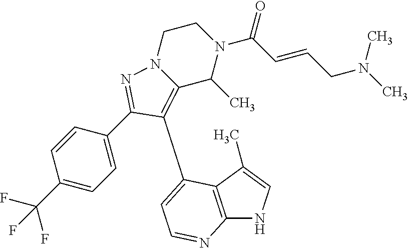 [SMILES: Cc1c[nH]c2nccc(-c3c(-c4ccc(C(F)(F)F)cc4)nn4c3C(C)N(C(=O)/C=C/CN(C)C)CC4)c12]

To a solution of (2E)-4-(dimethylamino)but-2-enoic acid hydrogen chloride (1/1) (44.4 mg, 0.268 mmol) in dichloromethane (3.0 ml) were added N,N-diisopropylethylamine (0.12 ml, 0.710 mmol) and O-(7-azabenzotriazol-1-yl)-N,N,N,N-tetramethyl uronium hexafluorophosphate (102 mg, 0.268 mmol) at 20° C. The mixture was stirred at 20° C. for 0.1 hour. To the mixture above was added 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 2) (80.0 mg, 0.179 mmol, Intermediate 465) at 20° C. The mixture was stirred at 20° C. for 2 hours. The reaction solution was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 13%-43% B; flow 25 ml/min; temperature: RT; Detector: UV 220/254 nm] to give 4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one (isomer 2) (8.00 mg, 0.0147 mmol, 96% purity, 8% yield) as a white solid.

LC-MS (Method 16): Rₜ=0.783 min; MS (ESIpos): m/z=523.2 [M+H]⁺.

¹H NMR (400 MHz, DMSO-d6), δ [ppm]=11.35-11.17 (m, 1H), 8.32-8.21 (m, 1H), 7.57-7.48 (m, 4H), 7.23-7.12 (m, 1H), 7.01-6.93 (m, 1H), 6.91-6.81 (m, 1H), 6.71-6.59 (m, 1H), 5.60-5.22 (m, 1H), 4.44 (d, J=12.4 Hz, 2H), 4.33-4.16 (m, 1H), 3.78-3.59 (m, 3H), 2.68-2.60 (m, 6H), 1.73 (d, J=6.8 Hz, 3H), 1.20-0.97 (m, 3H).

### Example 254

### (2E)-4-(dimethylamino)-1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one

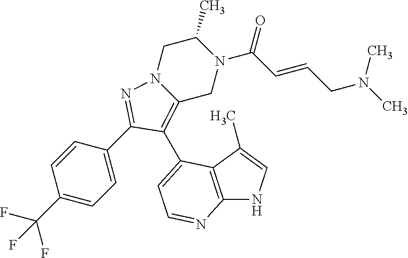 [SMILES: Cc1c[nH]c2nccc(-c3c(-c4ccc(C(F)(F)F)cc4)nn4c3CN(C(=O)/C=C/CN(C)C)[C@@H](C)C4)c12]

To a solution of (2E)-4-(dimethylamino)but-2-enoic acid hydrogen chloride (1/1) (55.5 mg, 0.335 mmol) in dichloromethane (6.0 ml) were added N,N-diisopropylethylamine (0.16 ml, 0.890 mmol) and O-(7-azabenzotriazol-1-yl)-N,N,N,N-tetramethyl uronium hexafluorophosphate (170 mg, 0.447 mmol) at 20° C. The mixture was stirred at 20° C. for 0.1 hour. To the mixture was added (6S)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (100 mg, 0.223 mmol, Intermediate 452) and the reaction mixture was stirred at 20° C. for 2 hours. The reaction solution was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-E; Column: Phenomenex luna C18 150*25 mm*10 μm, eluent A: water (0.2% formic acid), eluent B: acetonitrile; gradient: 0-10 min 19-49% B; flow 25 ml/min; temperature: RT; Detector: UV 220/254 nm] to give (2E)-4-(dimethylamino)-1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one (15.5 mg, 0.0279 mmol, 94% purity, 12% yield) as a white solid.

LC-MS (Method 16): Rₜ=0.466 min; MS (ESIpos): m/z=523.3 [M+H]⁺.

¹H NMR (400 MHz, DMSO-d6), δ [ppm]=11.58-11.44 (m, 1H), 8.30-8.20 (m, 1H), 7.60-7.51 (m, 4H), 7.23-7.16 (m, 1H), 7.00-6.91 (m, 1H), 6.79-6.55 (m, 2H), 4.99-4.82 (m, 1H), 4.57-4.24 (m, 3H), 4.17-3.85 (m, 1H), 3.09 (d, J=1.6 Hz, 2H), 2.23-2.10 (m, 6H), 1.76-1.66 (m, 3H), 1.29-1.16 (m, 3H).

### Example 255

### 4-(dimethylamino)-1-[2-(4-fluorophenyl)-4-methyl-3-(1H-pyrrolo[2,3-b]pyridin-4-yl)-6,7-dihydropyrazolo[1,5-a]pyrazin-5(4H)-yl]but-2-en-1-one (isomer 1)

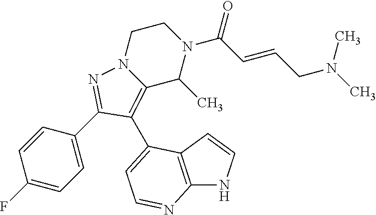 [SMILES: CC1c2c(-c3ccnc4[nH]ccc34)c(-c3ccc(F)cc3)nn2CCN1C(=O)/C=C/CN(C)C]

To a solution of 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 1) (80.0 mg, 0.208 mmol, Intermediate 457) and (2E)-4-(dimethylamino)but-2-enoic acid hydrogen chloride (1/1) (51.8 mg, 0.313 mmol) in N,N-dimethylformamide (16 ml) was added [bis(dimethylamino)methylene]-1H-1,2,3-triazolo[4,5-b]pyridinium-3-oxide hexafluorophosphate (119 mg, 0.313 mmol) and N,N-diisopropylethylamine (0.15 ml, 0.830 mmol) at 20° C. The mixture was stirred at 20° C. for 16 hours. The reaction solution was poured into water and extracted with ethyl acetate. The combined organic layers were washed with brine, dried over anhydrous sodium sulfate, filtered and the filtrate was concentrated in vacuo to give a residue. The residue was purified by preparative-HPLC (column: Phenomenex luna C 18 150*25 mm*10 μm; mobile phase: water (TFA)-ACN; B %: 14%-38%, 8 min) to give (2E)-4-(dimethylamino)-1-[2-(4-fluorophenyl)-4-methyl-3-(1H-pyrrolo[2,3-b]pyridin-4-yl)-6,7-dihydropyrazolo[1,5-a]pyrazin-5(4H)-yl]but-2-en-1-one (isomer 1) (51.7 mg, 0.110 mmol, 98% purity, 53% yield) as a white solid.

LC-MS (Method 16): Rₜ=0.605 min; MS (ESIpos): m/z=459.2 [M+H]⁺

¹H NMR (400 MHz, DMSO-d₆), δ [ppm]=11.69-11.37 (m, 1H), 8.25 (d, J=4.8 Hz, 1H), 8.14 (s, 1H), 7.42-7.26 (m, 3H), 7.05-6.87 (m, 3H), 6.74-6.51 (m, 2H), 6.03 (br s, 1H), 5.70-5.47 (m, 1H), 4.63-4.44 (m, 1H), 4.35 (dd, J=12.4, 2.8 Hz, 1H), 4.26-4.14 (m, 1H), 3.79-3.61 (m, 1H), 3.07 (d, J=5.2 Hz, 2H), 2.19 (s, 6H), 1.00 (d, J=6.4 Hz, 3H).

### Example 256

### (E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one (isomer 2)

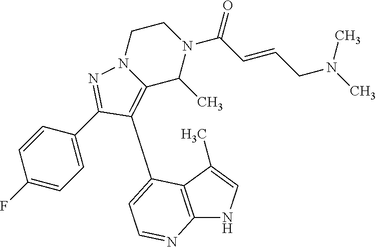 [SMILES: Cc1c[nH]c2nccc(-c3c(-c4ccc(F)cc4)nn4c3C(C)N(C(=O)/C=C/CN(C)C)CC4)c12]

To a solution of (2E)-4-(dimethylamino)but-2-enoic acid hydrogen chloride (1/1) (46.5 mg, 0.360 mmol) in dichloromethane (10.0 ml) were added N,N-diisopropylethylamine (0.14 ml, 0.830 mmol) and O-(7-azabenzotriazol-1-yl)-N,N,N,N-tetramethyl uronium hexafluorophosphate (158 mg, 0.415 mmol) at 20° C. The mixture was stirred at 20° C. for 0.1 hour. To the mixture above was added 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 2) (100 mg, 0.277 mmol, Intermediate 459) at 20° C. and the reaction mixture was stirred at 20° C. for 16 hours. The reaction solution was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 45%-75% B; flow 25 ml/min; temperature: RT; Detector: UV 220/254 nm] to give (E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one (isomer 2) (27.5 mg, 0.0550 mmol, 95% purity, 20% yield) as a white solid.

LC-MS (Method 16): Rₜ=0.749 min; MS (ESIpos): m/z=473.2 [M+H]⁺.

¹H NMR (400 MHz, DMSO-d6), δ [ppm]=11.56-11.37 (m, 1H), 8.31-8.17 (m, 1H), 7.39-7.27 (m, 2H), 7.26-7.11 (m, 1H), 7.09-6.99 (m, 2H), 6.99-6.90 (m, 1H), 6.85-6.71 (m, 1H), 6.70-6.59 (m, 1H), 5.61-5.34 (m, 1H), 4.48-4.30 (m, 2H), 4.28-4.16 (m, 1H), 3.93-3.72 (m, 1H), 3.09-2.89 (m, 2H), 2.20-2.00 (m, 6H), 1.78-1.64 (m, 3H), 1.16-0.78 (m, 3H).

### Example 257

### (2E)-4-(dimethylamino)-1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one

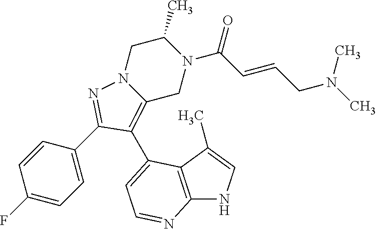 [SMILES: Cc1c[nH]c2nccc(-c3c(-c4ccc(F)cc4)nn4c3CN(C(=O)/C=C/CN(C)C)[C@@H](C)C4)c12]

To a solution of (2E)-4-(dimethylamino)but-2-enoic acid (19.5 mg, 151 μmol) in dichloromethane (2.0 ml) were added N,N-diisopropylethylamine (0.07 ml, 0.400 mmol) and O-(7-azabenzotriazol-1-yl)-N,N,N,N-tetramethyl uronium hexafluorophosphate (76.4 mg, 0.201 mmol) at 20° C. and the mixture was stirred at 20° C. for 0.1 hour. To the mixture above was added (6S)-2-(4-fluorophenyl)-6-methyl-3-(3-methyl-1H-pyrrolo[2,3-b]pyridin-4-yl)-4,5,6,7-tetrahydropyrazolo[1,5-a]pyrazine hydrogen chloride (1/1) (40.0 mg, 0.101 mmol, Intermediate 469) and the reaction mixture was stirred at 20° C. for 3 hours. The reaction solution was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 3%-29% B; flow 25 ml/min; temperature: RT; Detector: UV 220/254 nm] to give (2E)-4-(dimethylamino)-1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one (6.10 mg, 0.0126 mmol, 97% purity, 13% yield) as a white solid.

LC-MS (Method 16): Rₜ=0.612 min; MS (ESIpos): m/z=473.3 [M+H]⁺.

¹H NMR (400 MHz, DMSO-d6), δ [ppm]=11.28-11.15 (m, 1H), 8.22 (t, J=4.4 Hz, 1H), 7.39 (d, J=6.0 Hz, 2H), 7.14 (br s, 1H), 6.99 (t, J=8.8 Hz, 2H), 6.90 (dd, J=8.8, 4.8 Hz, 1H), 6.69-6.51 (m, 2H), 5.09-4.92 (m, 1H), 4.90-4.69 (m, 1H), 4.42-4.32 (m, 1H), 4.31-4.24 (m, 1H), 4.22-4.00 (m, 1H), 3.10-3.06 (m, 2H), 2.19 (d, J=10.4 Hz, 6H), 1.81-1.70 (m, 3H), 1.29-1.18 (m, 3H).

### Example 258

### 1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one (isomer 1)

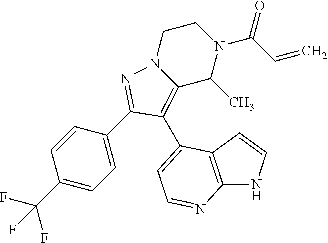 [SMILES: C=CC(=O)N1CCn2nc(-c3ccc(C(F)(F)F)cc3)c(-c3ccnc4[nH]ccc34)c2C1C]

To a solution of 4-methyl-3-(1H-pyrrolo[2,3-b]pyridin-4-yl)-2-(4-(trifluoromethyl)phenyl)-4,5,6,7-tetrahydropyrazolo[1,5-a]pyrazine hydrogen chloride (1/1) (isomer 1) (200 mg, 0.461 mmol, Intermediate 471) in dichloromethane (20 ml) were added prop-2-enoyl chloride (41.7 mg, 0.461 mmol) and triethylamine (0.19 ml, 1.40 mmol) at −10° C. The mixture was stirred at −10° C. for 1 hour. The reaction mixture was concentrated under reduced pressure to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 34%-64% B; flow 25 ml/min; temperature: RT; Detector: UV 220/254 nm] to give the title compound (40.5 mg, 0.0849 mmol, 95% purity, 18% yield) as a white solid.

LC-MS (Method 16): Rₜ=0.833 min; MS (ESIpos): m/z=452.2 [M+H]⁺.

¹H NMR (400 MHz, DMSO-d6), δ [ppm]=11.97-11.67 (m, 1H), 8.41-8.15 (m, 1H), 7.74-7.44 (m, 5H), 7.42-7.21 (m, 1H), 6.95 (dd, J=16.4, 10.4 Hz, 1H), 6.27-6.10 (m, 1H), 5.85-5.67 (m, 2H), 4.88-4.72 (m, 1H), 4.50-4.27 (m, 2H), 3.92-3.76 (m, 1H), 3.54-3.43 (m, 1H), 1.04-0.80 (m, 3H).

### Example 259

### 1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one (isomer 1)

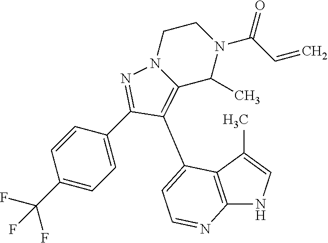 [SMILES: C=CC(=O)N1CCn2nc(-c3ccc(C(F)(F)F)cc3)c(-c3ccnc4[nH]cc(C)c34)c2C1C]

To a solution of 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 1) (45.0 mg, 0.101 mmol, Intermediate 474) in dichloromethane (5 ml) were added prop-2-enoyl chloride (9.09 mg, 0.101 mmol) and triethylamine (0.04 ml, 0.301 mmol) at −10° C. The mixture was stirred at −10° C. for 1 hour. The reaction was concentrated under reduced pressure to give a residue. The residue was purified by preparative HPLC [Instrument: ACSWH-GX-L; Column: Phenomenex luna C18 150*25 mm*10 μm, eluent A: water (0.2% formic acid), eluent B: acetonitrile; gradient: 0-10 min 24%-54% B; flow 25 ml/min; temperature: RT; Detector: UV 220/254 nm] to give 1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one (isomer 1) (7.80 mg, 0.0163 mmol, 98% purity, 16% yield) as a white solid.

LC-MS (Method 16): Rₜ=0.763 min; MS (ESIpos): m/z=466.2 [M+H]⁺.

¹H NMR (400 MHz, DMSO-d6), δ [ppm]=11.63-11.39 (m, 1H), 8.32-8.20 (m, 1H), 7.63-7.46 (m, 4H), 7.30-7.12 (m, 1H), 7.02-6.89 (m, 1H), 6.87-6.73 (m, 1H), 6.17 (d, J=16.4 Hz, 1H), 5.82-5.70 (m, 1H), 5.65-5.36 (m, 1H), 5.06-4.73 (m, 1H), 4.52-4.23 (m, 2H), 3.95-3.77 (m, 1H), 1.80-1.63 (m, 3H), 1.26-0.86 (m, 3H).

### Example 260

### 1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one

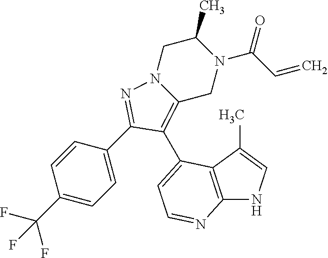 [SMILES: C=CC(=O)N1Cc2c(-c3ccnc4[nH]cc(C)c34)c(-c3ccc(C(F)(F)F)cc3)nn2C[C@H]1C]

To a solution of (6R)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine-hydrogen chloride (1/1) (100 mg, 0.223 mmol, Intermediate 478) and N,N-diisopropylethylamine (0.12 ml, 0.670 mmol) in dichloromethane was added prop-2-enoyl chloride (20.2 mg, 0.223 mmol) at 20° C. and the mixture was stirred at 20° C. for 1 hour. The reaction mixture was concentrated under vacuum to give a residue and the residue was purified by preparative NPLC [Instrument: CASWH-Prep-NPLC-E; Column: Welch Ultimate XB—SIOH 250*70*10 μm, eluent A: hexane, eluent B: ethanol; gradient: 0-15 min 10-50% B; flow 140 ml/min; temperature: RT; Detector: UV 220/254 nm] to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 36-66% B; flow 25 ml/min; temperature: RT; Detector: UV 220/254 nm] to give 1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one (14.6 mg, 0.0311 mmol, 99% purity, 14% yield) as a white solid.

LC-MS (Method 16): Rₜ=0.863 min; MS (ESIpos): m/z=466.2 [M+H]⁺.

¹H NMR (400 MHz, DMSO-d₆), δ [ppm]=11.49 (s, 1H), 8.25 (dd, J=7.2, 4.8 Hz, 1H), 7.64-7.47 (m, 4H), 7.19 (s, 1H), 7.02-6.65 (m, 2H), 6.14 (d, J=16.8 Hz, 1H), 5.74 (d, J=3.6 Hz, 1H), 5.29-4.69 (m, 2H), 4.54-3.87 (m, 3H), 1.80-1.64 (m, 3H), 1.34-1.14 (m, 3H).

### Example 261

### 1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one (isomer 2)

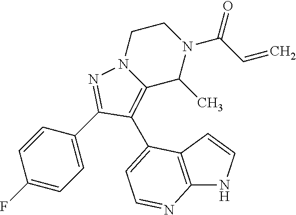 [SMILES: C=CC(=O)N1CCn2nc(-c3ccc(F)cc3)c(-c3ccnc4[nH]ccc34)c2C1C]

To a solution of 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 2) (120 mg, 0.313 mmol, Intermediate 481) in dichloromethane (2.0 ml) were added prop-2-enoyl chloride (28.3 mg, 0.313 mmol) and trimethylamine (0.13 ml, 0.940 mmol) at −10° C. The mixture was stirred at −10° C. for 1 hour. The reaction mixture was concentrated under reduced pressure to give a residue. The residue was purified by preparative HPLC [Instrument: ACSWH-GX-L; Column: Phenomenex luna C18 150*25 mm*10 μm, eluent A: water (0.2% formic acid), eluent B: acetonitrile; gradient: 0-10 min 24%-54% B; flow 25 ml/min; temperature: RT; Detector: UV 220/254 nm] to give 1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one (isomer 2) (42.8 mg, 0.103 mmol, 97% purity, 33% yield) as a white solid.

LC-MS (Method 16): Rₜ=0.670 min; MS (ESIpos): m/z=402.2 [M+H]⁺

¹H NMR (400 MHz, DMSO-d6), δ [ppm]=12.06-11.60 (m, 1H), 8.39-8.13 (m, 1H), 7.51 (br s, 1H), 7.29 (dd, J=8.8, 5.6 Hz, 3H), 7.08-7.01 (m, 2H), 6.95 (dd, J=16.8, 10.8 Hz, 1H), 6.23-6.14 (m, 1H), 5.82-5.70 (m, 2H), 4.52-4.39 (m, 1H), 4.39-4.20 (m, 2H), 4.18-4.05 (m, 1H), 3.91-3.75 (m, 1H), 1.01-0.87 (m, 3H).

### Example 262

### 1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one (isomer 1)

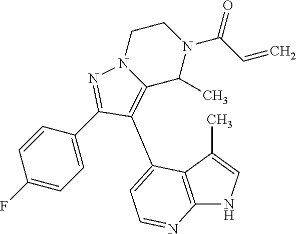 [SMILES: C=CC(=O)N1CCn2nc(-c3ccc(F)cc3)c(-c3ccnc4[nH]cc(C)c34)c2C1C]

To a solution of 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 1) (100 mg, 0.277 mmol, Intermediate 483) in dichloromethane (8.0 ml) were added trimethylamine (0.12 ml, 0.830 mmol) and a solution of prop-2-enoyl chloride (25.0 mg, 0.277 mmol) in dichloromethane (2.0 ml) at −10° C. The mixture was stirred at −10° C. for 1 hour. The reaction mixture was concentrated under reduced pressure to give a residue. The residue was purified by preparative HPLC [Instrument: ACSWH-PREP-NPLC-A; Column: Welch Ultimate XB—CN 250*50*10 μm, eluent A: Hexane, eluent B: ethanol; gradient: 0-15 min 5%-45% B; flow 100 ml/min; temperature: RT; Detector: UV 220/254 nm] to give 1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one (isomer 1) (9.00 mg, 0.0212 mmol, 98% purity, 8% yield) as a yellow solid.

LC-MS (Method 16): Rₜ=0.687 min; MS (ESIpos): m/z=416.3 [M+H]⁺

¹H NMR (400 MHz, DMSO-d6), δ [ppm]=11.32-11.13 (m, 1H), 8.31-8.15 (m, 1H), 7.41-7.31 (m, 2H), 7.22-7.09 (m, 1H), 7.02-6.94 (m, 2H), 6.86-6.68 (m, 2H), 6.14 (ddd, J=16.8, 9.6, 2.0 Hz, 1H), 5.76-5.67 (m, 1H), 5.56-5.20 (m, 1H), 4.62-4.43 (m, 1H), 4.37 (d, J=12.4 Hz, 1H), 4.26-4.10 (m, 1H), 3.88-3.63 (m, 1H), 1.73 (d, J=7.6 Hz, 3H), 1.17-0.94 (m, 3H).

### Example 263

### 1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one

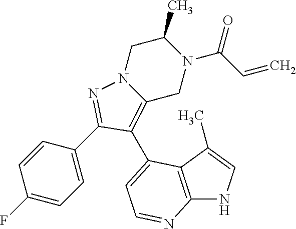 [SMILES: C=CC(=O)N1Cc2c(-c3ccnc4[nH]cc(C)c34)c(-c3ccc(F)cc3)nn2C[C@H]1C]

To a solution of (6R)-2-(4-fluorophenyl)-6-methyl-3-(3-methyl-1H-pyrrolo[2,3-b]pyridin-4-yl)-4,5,6,7-tetrahydropyrazolo[1,5-a]pyrazine (150 mg, 415 μmol, Intermediate 487) and N,N-diisopropylethylamine (0.07 ml, 420 μmol) in dichloromethane (10 ml) was added prop-2-enoyl chloride (37.6 mg, 415 μmol) at 20° C. and the reaction mixture was stirred at 20° C. for 1 hour. The reaction mixture was concentrated under vacuum to give a residue and the residue was purified by prep-TLC (ethyl acetate:methanol=20:1) to give a crude product. The crude product was purified by preparative HPLC [Instrument: ACSWH-GX-J; Column: Phenomenex luna C18 150*25 mm*10 μm, eluent A: water (0.2% formic acid), eluent B: acetonitrile; gradient: 0-10 min 22-52% B; flow 25 ml/min; temperature: RT; Detector: UV 220/254 nm] to give 1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one (11.9 mg, 27.1 μmol, 95% purity, 7% yield) as a white solid.

LC-MS (Method 20): Rₜ=0.898 min; MS (ESIpos): m/z=416.2 [M+H]⁺.

¹H NMR (400 MHz, DMSO-d₆), δ [ppm]=11.46 (s, 1H), 8.23 (dd, J=6.4, 4.8 Hz, 1H), 7.36 (t, J=6.4 Hz, 2H), 7.17 (s, 1H), 7.03 (t, J=8.8 Hz, 2H), 6.97-6.71 (m, 2H), 6.14 (d, J=16.4 Hz, 1H), 5.85-5.58 (m, 1H), 4.87 (d, J=17.6 Hz, 2H), 4.50-3.82 (m, 3H), 1.78-1.65 (m, 3H), 1.30-1.12 (m, 3H).

### Example 264

### (E)-4-(dimethylamino)-1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one (isomer 1)

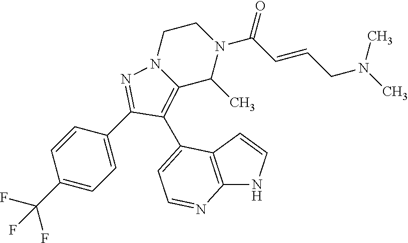 [SMILES: CC1c2c(-c3ccnc4[nH]ccc34)c(-c3ccc(C(F)(F)F)cc3)nn2CCN1C(=O)/C=C/CN(C)C]

To a solution of (2E)-4-(dimethylamino)but-2-enoic acid hydrogen chloride (1/1) (115 mg, 0.691 mmol) in dichloromethane (5.0 ml) were added N,N-diisopropylethylamine (0.32 ml, 1.84 mmol) and O-(7-azabenzotriazol-1-yl)-N,N,N,N-tetramethyl uronium hexafluorophosphate (263 mg, 0.691 mmol) at 20° C. and the mixture was stirred at 20° C. for 0.1 hour. To the mixture above was added 4-methyl-3-(1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 1) (200 mg, 0.461 mmol, Intermediate 471) at 20° C. The resulting mixture was stirred at 20° C. for 16 hours. The reaction mixture was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 13%-49% B; flow 25 ml/min; temperature: RT; Detector: UV 220/254 nm] to give the title compound (88.7 mg, 0.170 mmol, 97% purity, 37% yield) as a white solid.

LC-MS (Method 16): Rₜ=0.766 min; MS (ESIpos): m/z=509.2 [M+H]⁺

¹H NMR (400 MHz, DMSO-d6), δ [ppm]=12.05-11.67 (m, 1H), 8.43-8.17 (m, 1H), 7.68-7.43 (m, 5H), 7.41-7.16 (m, 1H), 6.99 (d, J=14.4 Hz, 1H), 6.73-6.55 (m, 1H), 5.87-5.68 (m, 1H), 4.88-4.69 (m, 1H), 4.41 (d, J=11.2 Hz, 2H), 4.21-4.06 (m, 1H), 3.95-3.83 (m, 1H), 3.82-3.65 (m, 2H), 2.70 (s, 6H), 1.08-0.84 (m, 3H).

### Example 265

### (E)-4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one (isomer 1)

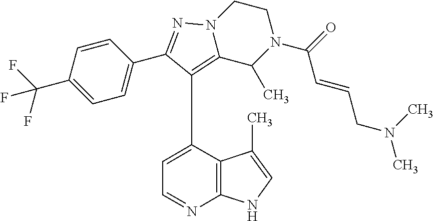 [SMILES: Cc1c[nH]c2nccc(-c3c(-c4ccc(C(F)(F)F)cc4)nn4c3C(C)N(C(=O)/C=C/CN(C)C)CC4)c12]

To a solution of (2E)-4-(dimethylamino)but-2-enoic acid hydrogen chloride (1/1) (22.2 mg, 0.134 mmol) in dichloromethane (5.0 ml) were added N,N-diisopropylethylamine (0.06 ml, 0.360 mmol) and O-(7-azabenzotriazol-1-yl)-N,N,N′,N′-tetramethyluronium Hexafluorophosphate (50.9 mg, 0.134 mmol) at 20° C. and the mixture was stirred at 20° C. for 0.1 hour. To the mixture above was added 4-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (isomer 1) (40.0 mg, 0.0893 mmol, Intermediate 474) at 20° C. The reaction mixture was stirred at 20° C. for 16 hours. The reaction solution was washed with water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-V; Column: Phenomenex luna C18 150*25 mm*10 μm, eluent A: water (0.2% formic acid), eluent B: acetonitrile; gradient: 0-10 min 12%-42% B; flow 30 ml/min; temperature: RT; Detector: UV 220/254 nm] to give the title compound (7.70 mg, 0.0147 mmol, 99% purity, 16% yield) as a white solid.

LC-MS (Method 16): Rₜ=0.675 min; MS (ESIpos): m/z=523.3 [M+H]⁺.

¹H NMR (400 MHz, DMSO-d6), δ [ppm]=11.36-11.15 (m, 1H), 8.31-8.19 (m, 1H), 7.57-7.46 (m, 4H), 7.22-7.10 (m, 1H), 7.01-6.81 (m, 1H), 6.71-6.53 (m, 2H), 5.60-5.17 (m, 1H), 4.65-4.45 (m, 1H), 4.41 (d, J=12.8 Hz, 1H), 4.29-4.13 (m, 1H), 3.79-3.65 (m, 1H), 2.26-2.17 (m, 6H), 1.75-1.69 (m, 3H), 1.16-0.95 (m, 3H).

### Example 266

### (2E)-4-(dimethylamino)-1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one

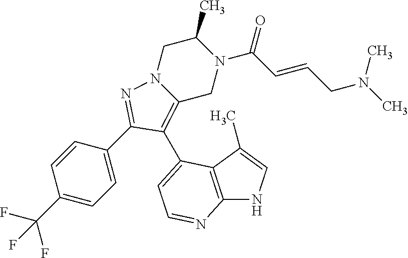 [SMILES: Cc1c[nH]c2nccc(-c3c(-c4ccc(C(F)(F)F)cc4)nn4c3CN(C(=O)/C=C/CN(C)C)[C@H](C)C4)c12]

To a solution of (2E)-4-(dimethylamino)but-2-enoic acid hydrogen chloride (1/1) (83.2 mg, 0.502 mmol) in dichloromethane (15 ml) were added and N,N-diisopropylethylamine (0.23 ml, 1.30 mmol) and 1-[bis(dimethylamino)methylene]-1H-1,2,3-triazolo[4,5-b]pyridinium-3-oxide hexafluorophosphate (255 mg, 0.670 mmol) at 20° C. and the mixture was stirred at 20° C. for 0.1 hour. To the mixture above was added (6R)-6-methyl-3-(3-methyl-1H-pyrrolo[2,3-b]pyridin-4-yl)-2-[4-(trifluoromethyl)phenyl]-4,5,6,7-tetrahydropyrazolo[1,5-a]pyrazine hydrogen chloride (1/1) (150 mg, 0.335 mmol, Intermediate 478) and the reaction mixture was stirred at 20° C. for 2 hours. The reaction mixture was concentrated under vacuum to give a residue and the residue was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 33-63% B; flow 25 ml/min; temperature: RT; Detector: UV 220/254 nm] to give (2E)-4-(dimethylamino)-1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one (29.5 mg, 0.0559 μmol, 99% purity, 17% yield) as a white solid.

LC-MS (Method 20): Rₜ=0.982 min; MS (ESIpos): m/z=523.2 [M+H]⁺1H NMR (400 MHz, DMSO-d₆), δ [ppm]=11.51 (s, 1H), 8.34-8.15 (m, 1H), 7.67-7.45 (m, 4H), 7.19 (s, 1H), 7.04-6.86 (m, 1H), 6.83-6.24 (m, 2H), 5.35-4.62 (m, 2H), 4.59-3.81 (m, 3H), 3.12-2.80 (m, 2H), 2.14 (s, 6H), 1.77-1.62 (m, 3H), 1.31-1.08 (m, 3H).

### Example 267

### (E)-4-(dimethylamino)-1-(2-(4-fluorophenyl)-4-methyl-3-(1H-pyrrolo[2,3-b]pyridin-4-yl)-6,7-dihydropyrazolo[1,5-a]pyrazin-5(4H)-yl) but-2-en-1-one (isomer 2)

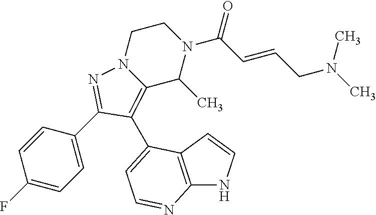 [SMILES: CC1c2c(-c3ccnc4[nH]ccc34)c(-c3ccc(F)cc3)nn2CCN1C(=O)/C=C/CN(C)C]

To a solution of (2E)-4-(dimethylamino)but-2-enoic acid hydrogen chloride (1/1) (64.7 mg, 0.391 μmol) in dichloromethane (3.0 ml) were added N,N-diisopropylethylamine (0.18 ml, 1.04 mmol) and O-(7-azabenzotriazol-1-yl)-N,N,N′,N′-tetramethyluronium hexafluorophosphate (149 mg, 0.391 mmol) at 20° C. and the mixture was stirred at 20° C. for 0.1 hour. To the mixture above was added 2-(4-fluorophenyl)-4-methyl-3-(1H-pyrrolo[2,3-b]pyridin-4-yl)-4,5,6,7-tetrahydropyrazolo[1,5-a]pyrazine hydrogen chloride (1/1) (isomer 2) (100 mg, 0.261 mmol, Intermediate 481) at 20° C. The reaction mixture was stirred at 20° C. for 16 hours. The reaction solution was poured into water and extracted with dichloromethane. The combined organic layers were washed with brine, dried over anhydrous sodium sulfate, filtered and concentrated in vacuo to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 7%-40% B; flow 25 ml/min; temperature: RT; Detector: UV 220/254 nm] to give (E)-4-(dimethylamino)-1-(2-(4-fluorophenyl)-4-methyl-3-(1H-pyrrolo[2,3-b]pyridin-4-yl)-6,7-dihydropyrazolo[1,5-a]pyrazin-5(4H)-yl) but-2-en-1-one (isomer 2) (65.4 mg, 0.140 mmol, 98% purity, 54% yield) as a white solid.

LC-MS (Method 16): Rₜ=0.716 min; MS (ESIpos): m/z=459.2 [M+H]⁺.

¹H NMR (400 MHz, DMSO-d6), δ [ppm]=12.03-11.60 (m, 1H), 8.41-8.19 (m, 1H), 7.54-7.38 (m, 1H), 7.29 (dd, J=8.4, 5.6 Hz, 3H), 7.08-6.94 (m, 3H), 6.72-6.60 (m, 1H), 5.85-5.67 (m, 1H), 4.83-4.70 (m, 1H), 4.46-4.34 (m, 2H), 4.33-4.21 (m, 1H), 3.92-3.80 (m, 1H), 3.78-3.65 (m, 2H), 2.66 (br s, 6H), 1.07-0.89 (m, 3H).

### Example 268

### (E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one (isomer 1)

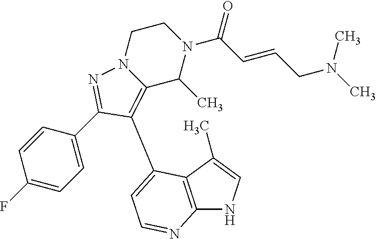 [SMILES: Cc1c[nH]c2nccc(-c3c(-c4ccc(F)cc4)nn4c3C(C)N(C(=O)/C=C/CN(C)C)CC4)c12]

To a solution of (2E)-4-(dimethylamino)but-2-enoic acid hydrogen chloride (1/1) (41.2 mg, 0.249 mmol) in dichloromethane (5.0 ml) were added N,N-diisopropylethylamine (0.09 ml, 0.500 mmol) and O-(7-azabenzotriazol-1-yl)-N,N,N′,N′-tetramethyluronium hexafluorophosphate (94.7 mg, 0.249 mmol) at 20° C. and the reaction mixture was stirred at 20° C. for 0.1 hour. To the mixture above was added 2-(4-fluorophenyl)-4-methyl-3-(3-methyl-1H-pyrrolo[2,3-b]pyridin-4-yl)-4,5,6,7-tetrahydropyrazolo[1,5-a]pyrazine (isomer 1) (60.0 mg, 166 μmol, Intermediate 483), the reaction mixture was stirred at 20° C. for 16 hours. The reaction solution was washed with water and extracted with dichloromethane.

The combined organic layers were washed with brine, dried over anhydrous sodium sulfate, filtered and concentrated in vacuo to give a residue. The residue was purified by preparative HPLC [Instrument: ACSWH-GX-Q; Column: Phenomenex luna C18 150*25 mm*10 μm, eluent A: water (0.2% formic acid), eluent B: acetonitrile; gradient: 0-10 min 7%-40% B; flow 25 ml/min; temperature: RT; Detector: UV 220/254 nm] to give (E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one (isomer 1) (9.80 mg, 0.0205 mmol, 99% purity, 12% yield) as a white solid.

LC-MS (Method 16): Rₜ=0.626 min; MS (ESIpos): m/z=473.2 [M+H]⁺.

¹H NMR (400 MHz, DMSO-d6), δ [ppm]=11.55-11.37 (m, 1H), 8.29-8.16 (m, 1H), 7.38-7.27 (m, 2H), 7.25-7.11 (m, 1H), 7.07-6.96 (m, 2H), 6.83-6.73 (m, 1H), 6.70-6.51 (m, 1H), 5.63-5.32 (m, 1H), 4.52-4.30 (m, 2H), 4.27-4.17 (m, 1H), 3.91-3.75 (m, 1H), 3.20-3.02 (m, 2H), 2.21 (br s, 6H), 1.77-1.64 (m, 3H), 1.14-0.80 (m, 3H).

### Example 269

### (2E)-4-(dimethylamino)-1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one

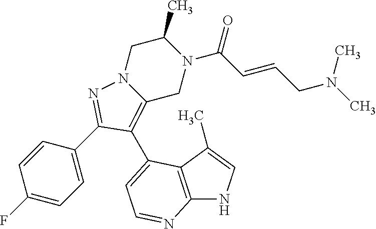 [SMILES: Cc1c[nH]c2nccc(-c3c(-c4ccc(F)cc4)nn4c3CN(C(=O)/C=C/CN(C)C)[C@H](C)C4)c12]

To a solution of (2E)-4-(dimethylamino)but-2-enoic acid-hydrogen chloride (1/1) (103 mg, 0.623 mmol) in dichloromethane (10 ml) were added and N,N-diisopropylethylamine (0.22 ml, 1.20 mmol) and 1-[bis(dimethylamino)methylene]-1H-1,2,3-triazolo[4,5-b]pyridinium-3-oxide hexafluorophosphate (316 mg, 0.830 mmol) at 20° C. and the mixture was stirred at 20° C. for 0.1 hour. To the mixture above was added (6R)-2-(4-fluorophenyl)-6-methyl-3-(3-methyl-1H-pyrrolo[2,3-b]pyridin-4-yl)-4,5,6,7-tetrahydropyrazolo[1,5-a]pyrazine (150 mg, 0.415 mmol, Intermediate 487) and the reaction mixture was stirred at 20° C. for 2 hours. The reaction mixture was concentrated under vacuum to give a residue and the residue was purified by preparative NPLC [Instrument: ACSWH-PREP-NPLC-A; Column: Welch Ultimate XB—SiOH 250*50*10 μm, eluent A: hexane, eluent B: ethanol (0.1% NH₃H₂O); gradient: 0-15 min 5-45% B; flow 100 ml/min; temperature: RT; Detector: UV 220/254 nm] to give a crude product. The crude product was purified by preparative HPLC [Instrument: ACSWH-GX-A; Column: Waters Xbridge 150*25 mm*5 μm, eluent A: water (0.2% ammonia hydroxide), eluent B: acetonitrile; gradient: 0-9 min 25-55% B; flow 25 ml/min; temperature: RT; Detector: UV 220/254 nm] to give (2E)-4-(dimethylamino)-1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one (37.4 mg, 0.0776 mmol, 98% purity, 19% yield) as a white solid.

LC-MS (Method 20): Rₜ=0.900 min; MS (ESIpos): m/z=473.2 [M+H]⁺.

¹H NMR (400 MHz, DMSO-d₆), δ [ppm]=11.47 (s, 1H), 8.22 (t, J=5.2 Hz, 1H), 7.36 (s, 2H), 7.18 (s, 1H), 7.03 (t, J=8.8 Hz, 2H), 6.97-6.87 (m, 1H), 6.83-6.29 (m, 2H), 5.32-4.56 (m, 2H), 4.48-3.66 (m, 3H), 3.03 (s, 2H), 2.15 (d, J=7.6 Hz, 6H), 1.84-1.57 (m, 3H), 1.35-1.05 (m, 3H).

### Example 270

### 1-[(6S)-2-(4-chloro-2-fluorophenyl)-6-methyl-3-(pyridin-4-yl)-6,7-dihydropyrazolo[1,5-a]pyrazin-5(4H)-yl]prop-2-en-1-one

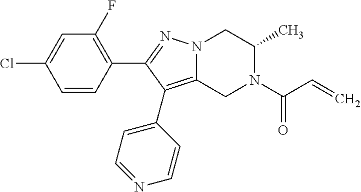 [SMILES: C=CC(=O)N1Cc2c(-c3ccncc3)c(-c3ccc(Cl)cc3F)nn2C[C@@H]1C]

To a solution of (6S)-2-(4-chloro-2-fluorophenyl)-6-methyl-3-(pyridin-4-yl)-4,5,6,7-tetrahydropyrazolo[1,5-a]pyrazine hydrogen chloride (1/1) (200 mg, 76% purity, 0.401 mmol, Intermediate 500) and prop-2-enoyl chloride (54.4 mg, 0.601 mmol) in dichloromethane (2.0 ml) was added triethylamine (0.2 ml, 1.30 mmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give 1-[(6S)-2-(4-chloro-2-fluorophenyl)-6-methyl-3-(pyridin-4-yl)-6,7-dihydropyrazolo[1,5-a]pyrazin-5(4H)-yl]prop-2-en-1-one (47.5 mg, 99% purity, 30% yield) as a colourless oil.

LC-MS (Method 20): Rₜ=0.906 min; MS (ESIpos): m/z=397.2 [M+H]⁺.

¹H NMR (400 MHz, DMSO-d₆), δ [ppm]=8.50 (d, J=5.6 Hz, 2H), 7.56-7.50 (m, 1H), 7.46 (dd, J=10.0, 2.0 Hz, 1H), 7.38 (dd, J=8.4, 2.0 Hz, 1H), 7.14 (d, J=6.0 Hz, 2H), 6.99-6.85 (m, 1H), 6.18 (dd, J=16.8, 2.0 Hz, 1H), 5.77 (d. J=12.8 Hz, 1H), 5.23 (d, J=17.6 Hz, 1H) 5.20-4.76 (m, 1H), 4.75-4.37 (m, 1H), 4.36-4.23 (m, 2H), 1.24 (s, 3H).

### Example 271

### 1-[(6R)-2-(4-chloro-2-fluorophenyl)-6-methyl-3-(pyridin-4-yl)-6,7-dihydropyrazolo[1,5-a]pyrazin-5(4H)-yl]prop-2-en-1-one

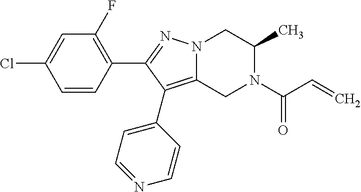 [SMILES: C=CC(=O)N1Cc2c(-c3ccncc3)c(-c3ccc(Cl)cc3F)nn2C[C@H]1C]

To a solution of (6R)-2-(4-chloro-2-fluorophenyl)-6-methyl-3-(pyridin-4-yl)-4,5,6,7-tetrahydropyrazolo[1,5-a]pyrazine hydrogen chloride (1/1) (200 mg, 81% purity, 0.427 mmol, Intermediate 503) and prop-2-enoyl chloride (58.0 mg, 0.641 mmol) in dichloromethane (2.0 ml) was added triethylamine (0.2 ml, 1.30 mmol) at 25° C. The mixture was stirred at 25° C. for 2 hours. The reaction mixture was poured into water and extracted with ethyl acetate. The combined organic phase was washed with brine, dried over anhydrous sodium sulfate, filtered and concentrated in vacuo to give a residue. The residue was purified by preparative-HPLC (Instrument: Gilson-281; Column: Phenomenex Luna C18 150*25 mm*10 μm; eluent A: 0.1% trifluoroacetic acid in water, eluent B: acetonitrile; gradient: 0-10 min 36-56% B; flow 25 ml/min; temperature: room temperature; Detector: UV 220/254 nm) to give 1-[(6R)-2-(4-chloro-2-fluorophenyl)-6-methyl-3-(pyridin-4-yl)-6,7-dihydropyrazolo[1,5-a]pyrazin-5(4H)-yl]prop-2-en-1-one (70.7 mg, 95% purity, 40% yield) as a colourless oil.

LC-MS (Method 20): Rₜ=0.898 min; MS (ESIpos): m/z=397.2 [M+H]⁺.

¹H NMR (400 MHz, DMSO-d6), δ [ppm]=8.50 (d, J=5.6 Hz, 2H), 7.56-7.50 (m, 1H), 7.46 (dd, J=10.0, 2.0 Hz, 1H), 7.38 (dd, J=8.4, 2.0 Hz, 1H), 7.15 (d, J=6.0 Hz, 2H), 6.99-6.85 (m, 1H), 6.18 (dd, J=16.8, 2.0 Hz, 1H), 5.76 (d, J=10.4 Hz, 1H), 5.23 (d, J=17.6 Hz, 1H) 5.17-4.82 (m, 1H), 4.79-4.41 (m, 1H), 4.39-4.24 (m, 2H), 1.24 (s, 3H).

### Example 272

### 1-[2-anilino-3-(pyridin-4-yl)-6,7-dihydropyrazolo[1,5-a]pyrazin-5(4H)-yl]prop-2-en-1-one

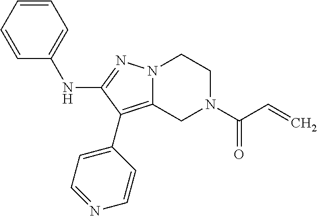 [SMILES: C=CC(=O)N1CCn2nc(Nc3ccccc3)c(-c3ccncc3)c2C1]

To a solution of N-phenyl-3-(pyridin-4-yl)-4,5,6,7-tetrahydropyrazolo[1,5-a]pyrazin-2-amine hydrogen chloride (1/2) (30.0 mg, 82.4 μmol, Intermediate 507) in THF (1 ml), prop-2-enoic acid (5.6 μl, 82 μmol) and N,N-diisopropylethylamine (43 μl, 250 μmol) were added at rt. T3P (57 μl, 50% purity in ethyl acetate, 96 μmol) was added and stirring at rt was continued for 18 h. Water (1 ml) was added, and the mixture was extracted with ethyl acetate twice. The combined organic layers were dried over sodium sulfate, filtered and concentrated under reduced pressure. The residue was purified by reverse phase preparative HPLC (Method 5) yielding 10.0 mg (91% purity, 32% yield) of the desired product.

LC-MS (Method 10): Rₜ=0.83 min; MS (ESIpos): m/z=346 [M+H]⁺

¹H-NMR (600 MHz, DMSO-d6) δ[ppm]: 8.62-8.47 (m, 2H), 8.08-7.94 (m, 1H), 7.46-7.34 (m, 2H), 7.20-7.08 (m, 4H), 7.00-6.87 (m, 1H), 6.74-6.65 (m, 1H), 6.22-6.15 (m, 1H), 5.83-5.72 (m, 1H), 5.05-4.83 (m, 2H), 4.19-4.05 (m, 4H)

### Example 273

### (2E)-1-[2-anilino-3-(pyridin-4-yl)-6,7-dihydropyrazolo[1,5-a]pyrazin-5(4H)-yl]-4-(dimethylamino)but-2-en-1-one

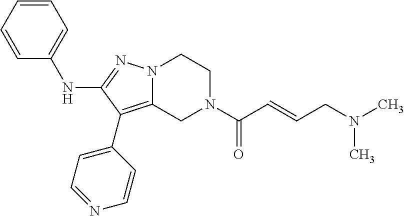 [SMILES: CN(C)C/C=C/C(=O)N1CCn2nc(Nc3ccccc3)c(-c3ccncc3)c2C1]

To a solution of N-phenyl-3-(pyridin-4-yl)-4,5,6,7-tetrahydropyrazolo[1,5-a]pyrazin-2-amine hydrogen chloride (1/2) (30.0 mg, 82.4 μmol, Intermediate 507) in THF (1 ml), (2E)-4-(dimethylamino)but-2-enoic acid hydrogen chloride (1/1) (13.6 mg, 82.4 μmol) and N,N-diisopropylethylamine (43 μl, 250 μl) were added at rt. T3P (74 μl, 50% purity in ethyl acetate, 130 μmol) was added and stirring at rt was continued for 16 h. Water (1 ml) was added, and the mixture was extracted with ethyl acetate twice. The combined organic layers were dried over sodium sulfate, filtered and concentrated under reduced pressure. The residue was purified by reverse phase preparative HPLC (Method 5), fractions obtained did not contain the desired product. Therefore, the aqueous phase was purified by reverse phase preparative HPLC (Method 5) yielding one fraction containing the desired compound (72 mg, 13%). This material was further purified by reverse phase preparative HPLC (Method 8) to yield 3.0 mg (99% purity, 9% yield) of the desired product.

LC-MS (Method 10): Rₜ=0.51 min; MS (ESIpos): m/z=403 [M+H]⁺

¹H-NMR (500 MHz, CHLOROFORM-d) δ [ppm]: 1.256 (0.45), 1.878 (0.97), 2.004 (1.07), 2.263 (5.10), 3.107 (2.25), 4.087 (0.74), 4.147 (0.50), 4.209 (2.91), 4.898 (1.13), 5.918 (3.20), 6.859 (0.66), 6.864 (1.14), 6.872 (1.47), 6.876 (1.82), 6.881 (1.58), 6.888 (1.25), 6.893 (0.88), 6.916 (0.63), 6.927 (1.20), 6.939 (0.74), 6.946 (0.66), 6.958 (1.07), 6.969 (0.56), 7.213 (1.25), 7.225 (16.00), 7.237 (6.52), 7.255 (4.43), 7.264 (11.52), 8.615 (2.82), 8.621 (2.83).

### Example 274

### 1-[2-(4-methoxyphenyl)-7-methyl-3-(pyridin-4-yl)-6,7-dihydropyrazolo[1,5-a]pyrazin-5(4H)-yl]prop-2-en-1-one (isomer 1)

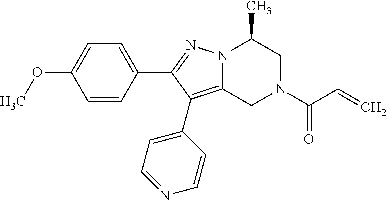 [SMILES: C=CC(=O)N1Cc2c(-c3ccncc3)c(-c3ccc(OC)cc3)nn2[C@@H](C)C1]

Under Argon, 2-(4-methoxyphenyl)-7-methyl-3-(pyridin-4-yl)-4,5,6,7-tetrahydropyrazolo[1,5-a]pyrazine-hydrogen chloride (1/1) (isomer 1) (72 mg, 202 μmol, Intermediate 519) was dissolved in DMF (2.5 ml), N,N-diisopropylethylamine (211 μl, 1212 μmol; CAS-RN: [7087-68-5]), prop-2-enoic acid (17.5 mg, 242 μmol; CAS-RN: [79-10-7]) and finally T3P (177 μl, 50% purity in DMF, 303 μmol; CAS-RN: [68957-94-8]) were added and stirred at RT for 30 min. Water was added and the mixture was concentrated in vacuo. The residue was redissolved in 1 ml DMSO and purified on a 12 g Biotage Sfär C18 (A30 μm) column-eluent: water with 0.05% ammonia/MeCN 95-50%, yielding the title compound (60.0 mg (98% purity, 77% yield).

LC-MS (Method 1): Rₜ=0.65 min; MS (ESIpos): m/z=375 [M+H]⁺

¹H-NMR (400 MHz, DMSO-d6) δ [ppm]: 1.478 (1.68), 1.493 (1.41), 2.518 (0.64), 2.523 (0.44), 3.753 (16.00), 3.859 (0.44), 3.874 (0.44), 3.894 (0.47), 3.910 (0.44), 4.249 (0.22), 4.275 (0.18), 4.447 (0.33), 4.836 (1.12), 5.781 (0.30), 5.808 (0.29), 6.184 (0.36), 6.226 (0.31), 6.903 (2.36), 6.907 (0.86), 6.919 (0.97), 6.924 (2.66), 6.931 (0.36), 6.947 (0.21), 6.974 (0.21), 6.989 (0.19), 7.145 (0.88), 7.158 (1.11), 7.257 (1.18), 7.278 (0.91), 8.526 (1.32), 8.539 (0.96).

### Example 275

### 1-[2-(4-methoxyphenyl)-7-methyl-3-(pyridin-4-yl)-6,7-dihydropyrazolo[1,5-a]pyrazin-5(4H)-yl]prop-2-en-1-one (isomer 2)

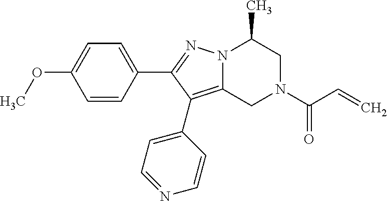 [SMILES: C=CC(=O)N1Cc2c(-c3ccncc3)c(-c3ccc(OC)cc3)nn2[C@@H](C)C1]

Under Argon, 2-(4-methoxyphenyl)-7-methyl-3-(pyridin-4-yl)-4,5,6,7-tetrahydropyrazolo[1,5-a]pyrazine-hydrogen chloride (1/1) (isomer 2) (49.2 mg, 138 μmol, Intermediate 518) was dissolved in DMF (1.7 ml), N,N-diisopropylethylamine (140 μl, 830 μmol; CAS-RN: [7087-68-5]), prop-2-enoic acid (11.9 mg, 166 μmol; CAS-RN: [79-10-7]) and finally T3P (120 μl, 50% purity, 210 μmol; CAS-RN: [68957-94-8]) were added and stirred at RT for 30 min. Water was added and the mixture was concentrated in vacuo. The residue was redissolved in 1 ml DMSO and purified on a 12 g Biotage Sfär C18 (A30 μm) column-eluent: water with 0.05% ammonia/MeCN 95-50%, yielding the title compound (39.0 mg (98% purity, 74% yield).

LC-MS (Method 1): Rₜ=0.65 min; MS (ESIpos): m/z=375 [M+H]⁺

¹H-NMR (400 MHz, DMSO-d6) δ [ppm]: 1.477 (1.71), 1.493 (1.44), 2.518 (0.64), 2.523 (0.41), 3.753 (16.00), 3.859 (0.44), 3.874 (0.45), 3.894 (0.47), 3.910 (0.44), 4.836 (1.14), 6.902 (2.40), 6.907 (0.88), 6.919 (0.99), 6.924 (2.69), 7.145 (0.90), 7.158 (1.13), 7.257 (1.20), 7.278 (0.92), 8.526 (1.33), 8.538 (0.97).

The following examples were produced analogously to the examples disclosed in this text. By sequential Suzuki coupling reactions with the given boronic acids, in combination with the stated starting material, followed by acid induced BOC deprotection (HCl), in a suitable solvent (Dioxane) and coupling with an acroyl chloride or acrylic acid in the presence of a base (DIPEA) and T3P. Purification by preparative HPLC was used to purify the products.

| | Structure |
|---|---|
| | IUPAC-Name |
| | LC-MS (method): Retention time; Mass found |
| Example | ¹H-NMr |
| Example 276 | 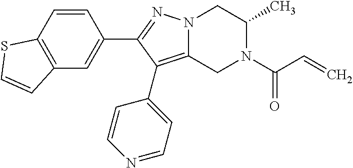 [SMILES: C=CC(=O)N1Cc2c(-c3ccncc3)c(-c3ccc4sccc4c3)nn2C[C@@H]1C] |
| | 1-[(6S)-2-(1-benzothiophen-5-yl)-6-methyl-3-(pyridin-4-yl)-6,7- |
| | dihydropyrazolo[1,5-a]pyrazin-5(4H)-yl]prop-2-en-1-one |
| | Produced from tert-butyl (6S)-2-bromo-3-iodo-6-methyl-6,7- |
| | dihydropyrazolo[1,5-a]pyrazine-5(4H)-carboxylate (Intermediate 466), |
| | pyridin-4-ylboronic acid and 2-(benzo[b]thiophen-5-yl)-4,4,5,5- |
| | tetramethyl-1,3,2-dioxaborolane |
| | LC-MS (Method 12): Rt = 0.910 min; MS (ESIpos): m/z = 401.2 [M + H]+. |
| | 1H NMR (400 MHz, DMSO-d6), δ [ppm] = 8.53 (d, J = 5.6 Hz, 2H), 7.96 |
| | (d, J = 8.4 Hz, 1H), 7.92 (s, 1H), 7.77 (d, J = 5.6 Hz, 1H), 7.43 (d, J = 5.6 |
| | Hz, 1H), 7.31 (d, J = 8.4 Hz, 1H), 7.24 (d, J = 5.6 Hz, 2H), 7.00-6.80 (m, |
| | 1H), 6.18 (dd, J = 16.8, 2.0 Hz, 1H) 5.77 (d, J = 11.6 Hz, 1H), 5.18 (d, J = |
| | 17.6 Hz, 1H), 5.10-4.63 (m, 1H), 4.62-4.38 (m, 1H), 4.37-4.20 (m, 2H), |
| | 1.26 (s, 3H). |
| Example 277 | 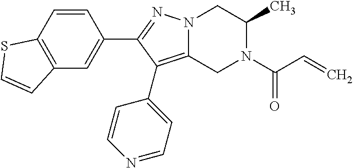 [SMILES: C=CC(=O)N1Cc2c(-c3ccncc3)c(-c3ccc4sccc4c3)nn2C[C@H]1C] |
| | 1-[(6R)-2-(1-benzothiophen-5-yl)-6-methyl-3-(pyridin-4-yl)-6,7- |
| | dihydropyrazolo[1,5-a]pyrazin-5(4H)-yl]prop-2-en-1-one |
| | Produced from tert-butyl (6R)-2-bromo-3-iodo-6-methyl-6,7- |
| | dihydropyrazolo[1,5-a]pyrazine-5(4H)-carboxylate (Intermediate 497), |
| | pyridin-4-ylboronic acid and and 2-(benzo[b]thiophen-5-yl)-4,4,5,5- |
| | tetramethyl-1,3,2-dioxaborolane |
| | LC-MS (Method 12): Rt = 0.915 min; MS (ESIpos): m/z = 401.2 [M + H]+. |
| | 1H NMR (400 MHz, DMSO-d6), δ [ppm] = 8.53 (d, J = 6.0 Hz, 2H), 7.96 |
| | (d, J = 8.4 Hz, 1H), 7.92 (d, J = 1.2 Hz, 1H), 7.77 (d, J = 5.2 Hz, 1H), |
| | 7.43 (d, J = 5.2 Hz, 1H), 7.30 (dd, J = 8.4, 1.2 Hz, 1H), 7.24 (d, J = 6.0 |
| | Hz, 2H), 7.02-6.77 (m, 1H), 6.18 (dd, J = 16.8, 2.0 Hz, 1H), 5.77 (d, J = |
| | 10.4 Hz, 1H), 5.18 (d, J = 17.6 Hz, 1H), 5.10-4.69 (m, 1H), 4.68-4.38 (m, |
| | 1H), 4.37-4.19 (m, 2H), 1.26 (s, 3H). |
| Example 278 | 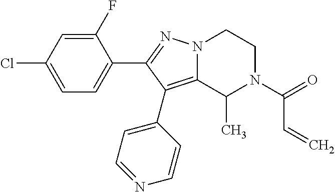 [SMILES: C=CC(=O)N1CCn2nc(-c3ccc(Cl)cc3F)c(-c3ccncc3)c2C1C] |
| | 1-[2-(4-chloro-2-fluorophenyl)-4-methyl-3-(pyridin-4-yl)-6,7- |
| | dihydropyrazolo[1,5-a]pyrazin-5(4H)-yl]prop-2-en-1-one (isomer 1) |
| | Produced from tert-butyl (4RS)-2-bromo-4-methyl-6,7- |
| | dihydropyrazolo[1,5-a]pyrazine-5(4H)-carboxylate (Intermediate 443) |
| | and (4-chloro-2-fluorophenyl)boronic acid followed by iodination (NIS) |
| | and a second Suzuki reaction using pyridin-4-ylboronic acid. Chiral |
| | preparative HPLC separation of the enantiomers was performed. |
| | LC-MS (Method 12): Rt = 0.903 min; MS (ESIpos): m/z = 397.1 [M + H]+. |
| | Optical rotation:[α]D = +219.4° (c = 0.143 g/100 ml, MeOH) |
| Example 279 | 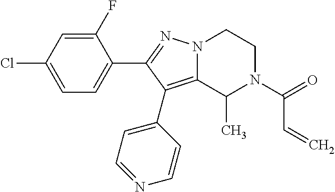 [SMILES: C=CC(=O)N1CCn2nc(-c3ccc(Cl)cc3F)c(-c3ccncc3)c2C1C] |
| | 1-[2-(4-chloro-2-fluorophenyl)-4-methyl-3-(pyridin-4-yl)-6,7- |
| | dihydropyrazolo[1,5-a]pyrazin-5(4H)-yl]prop-2-en-1-one (isomer 2) |
| | Produced from tert-butyl (4RS)-2-bromo-4-methyl-6,7- |
| | dihydropyrazolo[1,5-a]pyrazine-5(4H)-carboxylate (Intermediate 443) |
| | and (4-chloro-2-fluorophenyl)boronic acid followed by iodination (NIS) |
| | and a second Suzuki reaction using pyridin-4-ylboronic acid. Chiral |
| | preparative HPLC separation of the enantiomers was performed. |
| | LC-MS (Method 12): Rt = 0.912 min; MS (ESIpos): m/z = 397.1 [M + H]+. |
| | Optical rotation: [α]D = −223.0° (c = 0.159g/100 ml, MeOH) |
| Example 280 | 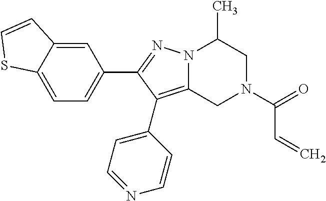 [SMILES: C=CC(=O)N1Cc2c(-c3ccncc3)c(-c3ccc4sccc4c3)nn2C(C)C1] |
| | 1-[(7RS)-2-(1-benzothiophen-5-yl)-7-methyl-3-(pyridin-4-yl)-6,7- |
| | dihydropyrazolo[1,5-a]pyrazin-5(4H)-yl]prop-2-en-1-one |
| | Produced from racemic tert-butyl-2-bromo-7-methyl-3-(pyridin-4-yl)-6,7- |
| | dihydropyrazolo[1,5-a]pyrazine-5(4H)-carboxylate (Intermediate 513) |
| | and 2-(1-benzothiophen-5-yl)-4,4,5,5-tetramethyl-1,3,2-dioxaborolane |
| | LC-MS (Method 12): Rt = 0.926 min; MS (ESIpos): m/z = 401.2 [M + H]+. |
| Example 281 | 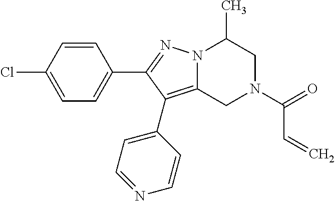 [SMILES: C=CC(=O)N1Cc2c(-c3ccncc3)c(-c3ccc(Cl)cc3)nn2C(C)C1] |
| | 1-[(7RS)-2-(4-chlorophenyl)-7-methyl-3-(pyridin-4-yl)-6,7- |
| | dihydropyrazolo[1,5-a]pyrazin-5(4H)-yl]prop-2-en-1-one |
| | Produced from racemic tert-butyl-2-bromo-7-methyl-3-(pyridin-4-yl)-6,7- |
| | dihydropyrazolo[1,5-a]pyrazine-5(4H)-carboxylate (Intermediate 513) |
| | and and (4-chlorophenyl)boronic acid |
| | LC-MS (Method 16): Rt = 0.439 min; MS (ESIpos): m/z = 379.0 [M + H]⁺. |
| Example 282 | 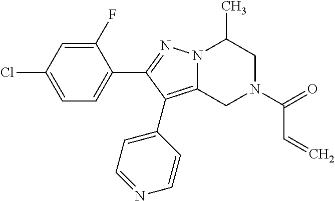 [SMILES: C=CC(=O)N1Cc2c(-c3ccncc3)c(-c3ccc(Cl)cc3F)nn2C(C)C1] |
| | 1-[2-(4-chloro-2-fluorophenyl)-7-methyl-3-(pyridin-4-yl)-6,7- |
| | dihydropyrazolo[1,5-a]pyrazin-5(4H)-yl]prop-2-en-1-one (isomer 1) |
| | Produced from racemic tert-butyl-2-bromo-7-methyl-3-(pyridin-4-yl)-6,7- |
| | dihydropyrazolo[1,5-a]pyrazine-5(4H)-carboxylate (Intermediate 513) |
| | and 2-(1-benzothiophen-5-yl)-4,4,5,5-tetramethyl-1,3,2-dioxaborolane |
| | and (4-chloro-2-fluorophenyl)boronic acid using chiral separation |
| | LC-MS (Method 12): Rt = 0.888 min; MS (ESIpos): m/z = 401.2 [M + H]+. |
| | Optical rotation:[α]D = −17.3º (c = 0.1464 g/100 ml, MeCN) |
| Example 283 | 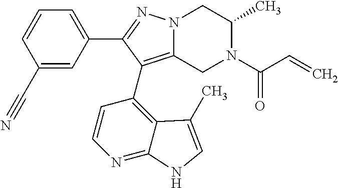 [SMILES: C=CC(=O)N1Cc2c(-c3ccnc4[nH]cc(C)c34)c(-c3cccc(C#N)c3)nn2C[C@@H]1C] |
| | 3-[(6S)-6-methyl-3-(3-methyl-1H-pyrrolo[2,3-b]pyridin-4-yl)-5-(prop- |
| | 2-enoyl)-4,5,6,7-tetrahydropyrazolo[1,5-a]pyrazin-2-yl]benzonitrile |
| | Produced from tert-butyl (6S)-2-bromo-3-iodo-6-methyl-6,7- |
| | dihydropyrazolo[1,5-a]pyrazine-5(4H)-carboxylate (Intermediate 492), 3- |
| | methyl-4-(4,4,5,5-tetramethyl-1,3,2-dioxaborolan-2-yl)-1H-pyrrolo[2,3- |
| | b]pyridine and (3-cyanophenyl)boronic acid |
| | LC-MS (Method 1): Rt = 0.86 min; MS (ESIpos): m/z = 423 [M + H]+. |
| | ¹H-NMR (400 MHz, DMSO-d6) δ [ppm]: 1.190 (3.26), 1.231 (1.93), 1.255 |
| | (2.94), 1.308 (0.52), 1.671 (13.72), 1.731 (16.00), 2.518 (6.04), 2.523 |
| | (3.94), 4.143 (0.44), 4.182 (0.59), 4.212 (0.99), 4.289 (1.83), 4.320 |
| | (3.66), 4.345 (2.51), 4.434 (1.00), 4.798 (0.52), 4.893 (3.82), 4.937 |
| | (4.25), 5.173 (1.39), 5.217 (1.39), 5.763 (2.14), 5.769 (2.02), 5.789 |
| | (1.46), 5.795 (1.48), 6.124 (1.56), 6.166 (1.73), 6.175 (1.93), 6.181 |
| | (1.44), 6.217 (0.98), 6.223 (0.93), 6.801 (0.79), 6.956 (1.86), 6.987 |
| | (5.98), 6.998 (5.72), 7.206 (5.77), 7.381 (3.63), 7.402 (7.82), 7.421 |
| | (4.92), 7.562 (4.20), 7.579 (3.24), 7.597 (1.06), 7.617 (2.12), 7.641 |
| | (6.00), 7.660 (5.35), 7.696 (3.12), 7.749 (0.95), 7.752 (1.33), 7.755 |
| | (0.93), 7.768 (0.75), 7.771 (1.01), 8.120 (0.90), 8.140 (0.82), 8.201 |
| | (1.39), 8.242 (6.10), 8.254 (6.13), 8.259 (7.74), 8.271 (6.88), 11.519 |
| | (3.62). |
| Example 284 | 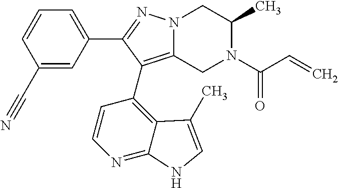 [SMILES: C=CC(=O)N1Cc2c(-c3ccnc4[nH]cc(C)c34)c(-c3cccc(C#N)c3)nn2C[C@H]1C] |
| | 3-[(6R)-6-methyl-3-(3-methyl-1H-pyrrolo[2,3-b]pyridin-4-yl)-5-(prop- |
| | 2-enoyl)-4,5,6,7-tetrahydropyrazolo[1,5-a]pyrazin-2-yl]benzonitrile |
| | Produced from tert-butyl (6R)-2-bromo-3-iodo-6-methyl-6,7- |
| | dihydropyrazolo[1,5-a]pyrazine-5(4H)-carboxylate (Intermediate 497) |
| | and 3-methyl-4-(4,4,5,5-tetramethyl-1,3,2-dioxaborolan-2-yl)-1H- |
| | pyrrolo[2,3-b]pyridine and (3-cyanophenyl)boronic acid |
| | LC-MS (Method 1): Rt = 0.86 min; MS (ESIpos): m/z = 423 [M + H]+. |
| | ¹H-NMR (400 MHz, DMSO-d6) δ [ppm]: 1.185 (3.32), 1.231 (1.78), 1.255 |
| | (2.70), 1.308 (0.56), 1.671 (12.13), 1.731 (16.00), 2.074 (0.64), 2.523 |
| | (3.21), 4.144 (0.42), 4.182 (0.74), 4.212 (1.29), 4.289 (1.72), 4.320 |
| | (3.52), 4.345 (2.60), 4.439 (1.02), 4.798 (0.51), 4.894 (3.73), 4.937 |
| | (4.13), 5.173 (1.80), 5.217 (1.78), 5.763 (2.32), 5.769 (2.14), 5.789 |
| | (1.68), 5.795 (1.72), 6.126 (1.54), 6.175 (2.15), 6.181 (1.63), 6.217 |
| | (1.23), 6.223 (1.16), 6.802 (1.05), 6.956 (1.92), 6.987 (4.87), 6.999 |
| | (4.52), 7.205 (5.64), 7.381 (3.27), 7.402 (7.15), 7.421 (4.63), 7.562 |
| | (3.97), 7.580 (3.16), 7.597 (1.30), 7.617 (2.58), 7.641 (5.63), 7.660 |
| | (4.95), 7.696 (3.07), 7.752 (1.69), 7.771 (1.32), 8.120 (1.19), 8.140 |
| | (1.07), 8.201 (1.83), 8.242 (4.81), 8.254 (5.01), 8.259 (6.90), 8.271 |
| | (6.32), 11.519 (3.52). |

The following examples were produced analogously to the examples disclosed in this text. By sequential Suzuki coupling reactions with the given boronic acids, in combination with the stated starting material, followed by acid induced BOC deprotection (HCl), in a suitable solvent (Dioxane) and coupling with (2E)-4-(dimethylamino)but-2-enoic acid in the presence of a base (DIPEA) and T3P or HATU. Purification by preparative HPLC was used to purify the products.

| | Structure |
|---|---|
| | IUPAC-Name |
| | LC-MS (method): Retention time: Mass found |
| Example | ¹H-NMR |
| Example 285 | 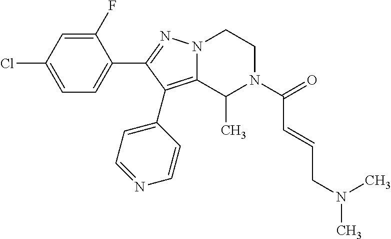 [SMILES: CC1c2c(-c3ccncc3)c(-c3ccc(Cl)cc3F)nn2CCN1C(=O)/C=C/CN(C)C] |
| | (2E)-1-[2-(4-chloro-2-fluorophenyl)-4-methyl-3-(pyridin-4-yl)-6,7- |
| | dihydropyrazolo[1,5-a]pyrazin-5(4H)-yl]-4-(dimethylamino)but-2-en- |
| | 1-one (isomer 1) |
| | Produced from (4RS)-2-bromo-4-methyl-6,7-dihydropyrazolo[1,5- |
| | alpyrazine-5(4H)-carboxylate (Intermediate 443), pyridin-4-ylboronic |
| | acid and (4-chloro-2-fluorophenyl)boronic acid with separation of the |
| | enantiomers by preparative chiral HPLC |
| | LC-MS (Method 12): Rt = 0.919 min; MS (ESIpos): m/z = 454.3 [M + H]+. |
| | Optical rotation: [α]D = +202.8° (c = 0.1758 g/100 ml, MeOH) |
| Example 286 | 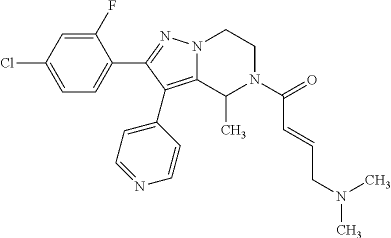 [SMILES: CC1c2c(-c3ccncc3)c(-c3ccc(Cl)cc3F)nn2CCN1C(=O)/C=C/CN(C)C] |
| | (2E)-1-[2-(4-chloro-2-fluorophenyl)-4-methyl-3-(pyridin-4-yl)-6,7- |
| | dihydropyrazolo[1,5-a]pyrazin-5(4H)-yl]-4-(dimethylamino)but-2-en- |
| | 1-one (isomer 2) |
| | Produced from (4RS)-2-bromo-4-methyl-6,7-dihydropyrazolo[1,5- |
| | alpyrazine-5(4H)-carboxylate (Intermediate 443), pyridin-4-ylboronic |
| | acid and (4-chloro-2-fluorophenyl)boronic acid with separation of the |
| | enantiomers by preparative chiral HPLC |
| | LC-MS (Method 12): Rt = 0.934 min; MS (ESIpos): m/z = 454.2 [M + H]+. |
| | Optical rotation:[α]D = - 241.3° (c = 0.0790 g/100 ml, MeOH) |
| Example 287 | 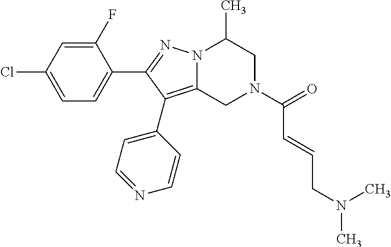 [SMILES: CC1CN(C(=O)/C=C/CN(C)C)Cc2c(-c3ccncc3)c(-c3ccc(Cl)cc3F)nn21] |
| | (2E)-1-[2-(4-chloro-2-fluorophenyl)-7-methyl-3-(pyridin-4-yl)-6,7- |
| | dihydropyrazolo[1,5-a]pyrazin-5(4H)-yl]-4-(dimethylamino)but-2-en- |
| | 1-one (isomer 1) |
| | Produced from tert-butyl-2-bromo-7-methyl-3-(pyridin-4-yl)-6,7- |
| | dihydropyrazolo[1,5-a]pyrazine-5(4H)-carboxylate (Intermediate 513) |
| | and (4-chloro-2-fluorophenyl)boronic acid using chiral separation |
| | LC-MS (Method 12): Rt = 0.932 min; MS (ESIpos): m/z = 454.3 [M + H]+. |
| | Optical rotation:[α]D = −12.84° (c = 0.2578 g/100 ml, MeOH) |
| Example 288 | 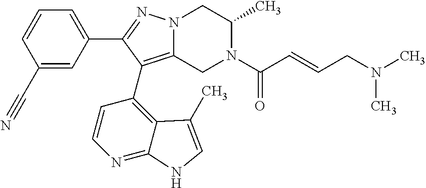 [SMILES: Cc1c[nH]c2nccc(-c3c(-c4cccc(C#N)c4)nn4c3CN(C(=O)/C=C/CN(C)C)[C@@H](C)C4)c12] |
| | 3-[(6S)-5-[(2E)-4-(dimethylamino)but-2-enoyl]-6-methyl-3-(3-methyl- |
| | 1H-pyrrolo[2,3-b]pyridin-4-yl)-4,5,6,7-tetrahydropyrazolo[1,5- |
| | a]pyrazin-2-yl]benzonitrile |
| | Produced from tert-butyl (6S)-2-bromo-3-iodo-6-methyl-6,7- |
| | dihydropyrazolo[1,5-a]pyrazine-5(4H)-carboxylate (Intermediate 492) 3- |
| | methyl-4-(4,4,5,5-tetramethyl-1,3,2-dioxaborolan-2-yl)-1H-pyrrolo[2,3- |
| | b]pyridine and (3-cyanophenyl)boronic acid |
| | LC-MS (Method 1): Rt = 0.64 min; MS (ESIpos): m/z = 480 [M + H]+. |
| | ¹H-NMR (400 MHz, DMSO-d6) δ [ppm]: 1.188 (2.00), 1.252 (1.59), 1.667 |
| | (6.70), 1.732 (12.70), 1.734 (13.24), 2.143 (6.11), 2.163 (16.00), 2.518 |
| | (4.02), 2.523 (2.42), 3.038 (1.72), 3.057 (2.21), 3.067 (1.71), 3.590 |
| | (0.53), 4.205 (0.55), 4.281 (1.00), 4.310 (2.23), 4.339 (1.57), 4.434 |
| | (0.64), 4.873 (2.92), 4.916 (2.85), 5.156 (0.80), 5.199 (0.82), 6.650 |
| | (1.06), 6.704 (1.01), 6.797 (0.56), 6.944 (1.04), 6.982 (2.76), 6.994 |
| | (2.63), 7.208 (3.19), 7.381 (2.25), 7.400 (4.87), 7.420 (3.10), 7.559 |
| | (2.23), 7.579 (1.74), 7.596 (0.61), 7.616 (1.24), 7.640 (3.83), 7.659 |
| | (3.48), 7.695 (1.76), 7.751 (0.82), 7.771 (0.63), 8.117 (0.56), 8.137 |
| | (0.49), 8.197 (0.92), 8.241 (3.55), 8.254 (5.58), 8.266 (4.03), 11.520 |
| | (2.60). |
| Example 289 | 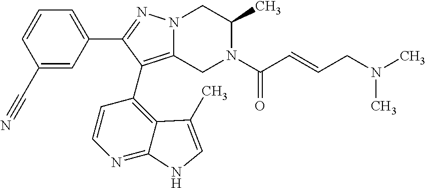 [SMILES: Cc1c[nH]c2nccc(-c3c(-c4cccc(C#N)c4)nn4c3CN(C(=O)/C=C/CN(C)C)[C@H](C)C4)c12] |
| | 3-[(6R)-5-[(2E)-4-(dimethylamino)but-2-enoyl]-6-methyl-3-(3-methyl- |
| | ¹H-pyrrolo[2,3-b]pyridin-4-yl)-4,5,6,7-tetrahydropyrazolo[1,5- |
| | a]pyrazin-2-yl]benzonitrile |
| | Produced from tert-butyl (6R)-2-bromo-3-iodo-6-methyl-6,7- |
| | dihydropyrazolo[1,5-a]pyrazine-5(4H)-carboxylate (Intermediate 497) 3- |
| | methyl-4-(4,4,5,5-tetramethyl-1,3,2-dioxaborolan-2-yl)-1H-pyrrolo[2,3- |
| | b]pyridine and (3-cyanophenyl)boronic acid |
| | LC-MS (Method 1): Rt = 0.64 min; MS (ESIpos): m/z = 480 [M + H]+. |
| | 1H-NMR (400 MHz, DMSO-d6) δ [ppm]: 1.187 (1.59), 1.250 (1.18), 1.667 |
| | (4.93), 1.734 (10.34), 2.142 (4.72), 2.163 (16.00), 2.518 (2.42), 2.523 |
| | (1.54), 3.036 (1.33), 3.057 (2.06), 3.067 (1.69), 3.590 (0.42), 4.205 |
| | (0.58), 4.281 (0.78), 4.310 (1.73), 4.337 (1.24), 4.439 (0.48), 4.873 |
| | (2.29), 4.916 (2.27), 5.156 (0.87), 5.199 (0.85), 6.651 (0.84), 6.701 |
| | (0.95), 6.797 (0.59), 6.944 (0.83), 6.982 (2.00), 6.994 (1.91), 7.208 |
| | (2.51), 7.381 (1.75), 7.400 (3.76), 7.420 (2.38), 7.559 (1.72), 7.579 |
| | (1.34), 7.596 (0.64), 7.616 (1.31), 7.636 (2.56), 7.640 (2.95), 7.659 |
| | (2.67), 7.694 (1.35), 7.751 (0.87), 7.754 (0.59), 7.767 (0.46), 7.770 |
| | (0.65), 8.118 (0.59), 8.137 (0.55), 8.197 (1.02), 8.241 (2.62), 8.254 |
| | (4.32), 8.266 (3.20), 11.520 (2.00). |

### Example 290

### 1-[(6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 1)

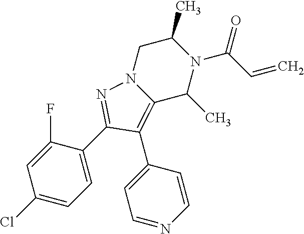 [SMILES: C=CC(=O)N1C(C)c2c(-c3ccncc3)c(-c3ccc(Cl)cc3F)nn2C[C@H]1C]

1-[(4RS,6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (210 mg, 511 μmol, Intermediate 531) was separated by chiral separation [Method details: “Column: Chiralpak IK-3 50×4.6 mm I.D., 3 μm Mobile phase: Phase A for CO2, and Phase B for EtOH (0.05% DEA); Gradient elution: 40% EtOH (0.05% DEA) in CO2; Flow rate: 3 ml/min; Detector: PDA; Column Temp: 35° C.; Back Pressure: 100 Bar”] to give 1-[(6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 1)

(9.80 mg, 22.4 μmol, 4% yield) as a white solid.

Optical rotation: [α]_{D}=+100.127° (c=0.2406 g/100 ml, methanol)

LC-MS (Method 11): Rₜ=0.457 min; MS (ESIpos): m/z=411.1 [M+H]⁺.

¹H NMR (400 MHz, DMSO-d6), δ [ppm]=8.54 (d, J=6.0 Hz, 2H), 7.54 (t, J=8.0 Hz, 1H), 7.45 (dd, J=10.0, 1.6 Hz, 1H), 7.38 (dd, J=8.4, 2.0 Hz, 1H), 7.13 (d, J=6.0 Hz, 2H), 6.89 (dd, J=16.4, 10.4 Hz, 1H), 6.23 (dd, J=16.4, 2.0 Hz, 1H), 5.77 (dd, J=10.4, 2.0 Hz, 1H), 5.38 (q, J=6.4 Hz, 1H), 4.86-4.77 (m, 1H), 4.75-4.67 (m, 1H), 4.41 (d, J=13.2 Hz, 1H), 1.48 (d, J=6.4 Hz, 3H), 0.90 (d, J=6.4 Hz, 3H).

### Example 291

### 1-[(6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 2)

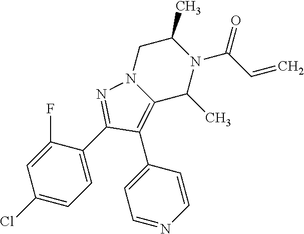 [SMILES: C=CC(=O)N1C(C)c2c(-c3ccncc3)c(-c3ccc(Cl)cc3F)nn2C[C@H]1C]

1-[(4RS,6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (210 mg, 511 μmol, Intermediate 531) was separated by chiral separation [Method details: “Column: Chiralpak IK-3 50×4.6 mm I.D., 3 μm Mobile phase: Phase A for CO2, and Phase B for EtOH (0.05% DEA); Gradient elution: 40% EtOH (0.05% DEA) in CO2; Flow rate: 3 ml/min; Detector: PDA; Column Temp: 35° C.; Back Pressure: 100 Bar”] to give 1-[(6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 2) (170 mg, 407 μmol, 80% yield) as a white solid.

Optical rotation: [α]_{D}=+170.077° (c=0.4364 g/100 ml, methanol)

LC-MS (Method 11): Rₜ=0.453 min; MS (ESIpos): m/z=411.1 [M+H]⁺.

¹H NMR (400 MHz, DMSO-d₆), δ [ppm]=8.53 (d, J=5.6 Hz, 2H), 7.50 (t, J=8.0 Hz, 1H), 7.42 (dd, J=10.0, 2.0 Hz, 1H), 7.35 (dd, J=8.4, 2.0 Hz, 1H), 7.30-7.15 (m, 2H), 7.06-6.86 (m, 1H), 6.31-6.13 (m, 1H), 5.94 (d, J=1.2 Hz, 1H), 5.79 (d, J=9.2 Hz, 1H), 5.40-4.81 (m, 1H), 4.45-4.22 (m, 2H), 1.34-1.14 (m, 6H).

### Example 292

### 1-[(6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 1)

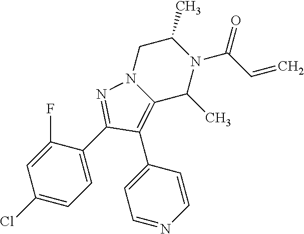 [SMILES: C=CC(=O)N1C(C)c2c(-c3ccncc3)c(-c3ccc(Cl)cc3F)nn2C[C@@H]1C]

1-[(4RS,6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (200 mg, 487 μmol, Intermediate 543) was separated by chiral separation [Method details: “Column: Chiralcel OJ-3 50×4.6 mm I.D., 3 μm Mobile phase: Phase A for CO2, and Phase B for EtOH (0.05% DEA); Gradient elution: EtOH (0.05% DEA) in CO2 from 5% to 40% Flow rate: 3 mL/min; Detector: PDA; Column Temp: 35 C; Back Pressure: 100 Bar”] to give 1-[(6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 1) (16.0 mg, 35.6 μmol, 7% yield) as a yellow solid.

Optical rotation: [α]_{D}=−83.766° (c=0.2168 g/100 ml, methanol)

LC-MS (Method 11): Rₜ=0.792 min; MS (ESIpos): m/z=411.1 [M+H]⁺.

¹H NMR (400 MHz, DMSO-d₆), δ [ppm]=8.53 (d, J=5.2 Hz, 2H), 7.53 (t, J=8.0 Hz, 1H), 7.44 (d, J=10.0 Hz, 1H), 7.37 (d, J=8.4 Hz, 1H), 7.12 (d, J=5.2 Hz, 2H), 6.88 (dd, J=16.4, 10.4 Hz, 1H), 6.23 (dd, J=16.4, 1.6 Hz, 1H), 5.77 (d, J=11.2 Hz, 1H), 5.38 (q, J=6.4 Hz, 1H), 4.80 (d, J=5.2 Hz, 1H), 4.70 (d, J=13.6 Hz, 1H), 4.40 (d, J=13.6 Hz, 1H), 1.48 (d, J=6.4 Hz, 3H), 0.89 (d, J=6.4 Hz, 3H).

### Example 293

### 1-[(6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 2)

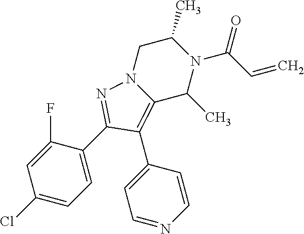 [SMILES: C=CC(=O)N1C(C)c2c(-c3ccncc3)c(-c3ccc(Cl)cc3F)nn2C[C@@H]1C]

1-[(4RS,6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (200 mg, 487 μmol, Intermediate 543) was separated by chiral separation [Method details: “Column: Chiralcel OJ-3 50×4.6 mm I.D., 3 μm Mobile phase: Phase A for CO2, and Phase B for EtOH (0.05% DEA); Gradient elution: EtOH (0.05% DEA) in CO2 from 5% to 40% Flow rate: 3 mL/min; Detector: PDA; Column Temp: 35 C; Back Pressure: 100 Bar”] to give 1-[(6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one (isomer 2) (124 mg, 297 μmol, 61% yield) as a white solid.

Optical rotation: [α]_{D}=−157.235° (c=0.3540 g/100 ml, methanol)

LC-MS (Method 11): Rₜ=0.793 min; MS (ESIpos): m/z=411.1 [M+H]⁺.

¹H NMR (400 MHz, DMSO-d₆), δ [ppm]=8.52 (d, J=5.6 Hz, 2H), 7.53-7.45 (m, 1H), 7.41 (dd, J=10.0, 2.0 Hz, 1H), 7.34 (dd, J=8.4, 2.0 Hz, 1H), 7.19 (br s, 2H), 7.05-6.84 (m, 1H), 6.31-6.11 (m, 1H), 6.00-5.72 (m, 2H), 5.00-4.79 (m, 1H), 4.43-4.20 (m, 2H), 1.30-1.15 (m, 6H)

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology”

(Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Experimental Section—Biological Assays

The pharmacological activity of the compounds according to the invention can be assessed using in vitro- and/or in vivo-assays, as known to the person skilled in the art. The following examples describe the biological activity of the compounds according to the invention, without the invention being limited to said examples.

Example compounds according to the invention were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of the compounds of the present invention can be demonstrated in the following assays:

### Expression and Purification of the EGFR Proteins Used in the Biochemical Kinase Assays

The different EGFR proteins used in the biochemical kinase activity inhibition assays were generated inhouse by expression in insect cells using Baculo Virus system and subsequent purification as described in the following paragraphs.

### Expression Constructs:

The cDNAs encoding the various protein sequences from human EGFR human (P00533) were optimized for expression in eukaryotic cells and synthesized by the GeneArt Technology at Life Technologies.

These DNA sequences encoded the following sequence:

Construct EGFR #1 amino acid R669 to A1210

Construct EGFR #2 amino acid R669 to A1210 and the insertion of the amino acids sequence ASV between V769 and D770

Construct EGFR #3 amino acid R669 to A1210 and the insertion of the amino acids sequence SVD between D770 and N771

Additionally all constructs EGFR #1 to #3 encoded: at the N-terminus a TEV (Tobacco etch virus) protease cleavage site (DYDIPTTENLYFQG), at the C-terminus two stop codons and additionally 5′ and 3′ att-DNA sequences for Gateway Cloning.

Each of the four EFGR constructs was subcloned using the Gateway Technology into the Destination vector pD-Ins1. The vector pD-Ins1 is a Baculovirus transfer vector (based on vector pVL1393, Pharmingen) which provides a N-terminal fusion of a GST-tag to the integrated gene construct. The respective transfer vectors were termed pD-Ins1_EGFR #1, pD-Ins1_EGFR #2, pD-Ins1_EGFR #3.

EGFR amino acid sequences:
GST-EGFR #1 (Wild Type)
MSPILGYWKIKGLVQPTRLLLEYLEEKYEEHLYERDEGDKWRNKKFELGLEFPNLPYYID
GDVKLTQSMAIIRYIADKHNMLGGCPKERAEISMLEGAVLDIRYGVSRIAYSKDFETLKVD
FLSKLPEMLKMFEDRLCHKTYLNGDHVTHPDFMLYDALDVVLYMDPMCLDAFPKLVCFK
KRIEAIPQIDKYLKSSKYIAWPLQGWQATFGGGDHPPKSDPITSLYKKAGSDYDIPTTTEN
LYFQGRRRHIVRKRTLRRLLQERELVEPLTPSGEAPNQALLRILKETEFKKIKVLGSGAFG
TVYKGLWIPEGEKVKIPVAIKELREATSPKANKEILDEAYVMASVDNPHVCRLLGICLTSTV
QLITQLMPFGCLLDYVREHKDNIGSQYLLNWCVQIAKGMNYLEDRRLVHRDLAARNVLV
KTPQHVKITDFGLAKLLGAEEKEYHAEGGKVPIKWMALESILHRIYTHQSDVWSYGVTVW
ELMTFGSKPYDGIPASEISSILEKGERLPQPPICTIDVYMIMVKCWMIDADSRPKFRELIIEF
SKMARDPQRYLVIQGDERMHLPSPTDSNFYRALMDEEDMDDVVDADEYLIPQQGFFSS
PSTSRTPLLSSLSATSNNSTVACIDRNGLQSCPIKEDSFLQRYSSDPTGALTEDSIDDTFL
PVPEYINQSVPKRPAGSVQNPVYHNQPLNPAPSRDPHYQDPHSTAVGNPEYLNTVQPT
CVNSTFDSPAHWAQKGSHQISLDNPDYQQDFFPKEAKPNGIFKGSTAENAEYLRVAPQS
SEFIGA
GST-EGFR #2 (ASV between V769 and D770)
MSPILGYWKIKGLVQPTRLLLEYLEEKYEEHLYERDEGDKWRNKKFELGLEFPNLPYYID
GDVKLTQSMAIIRYIADKHNMLGGCPKERAEISMLEGAVLDIRYGVSRIAYSKDFETLKVD
FLSKLPEMLKMFEDRLCHKTYLNGDHVTHPDFMLYDALDVVLYMDPMCLDAFPKLVCFK
KRIEAIPQIDKYLKSSKYIAWPLQGWQATFGGGDHPPKSDPITSLYKKAGSDYDIPTTTEN
LYFQGRRRHIVRKRTLRRLLQERELVEPLTPSGEAPNQALLRILKETEFKKIKVLGSGAFG
TVYKGLWIPEGEKVKIPVAIKELREATSPKANKEILDEAYVMASVDASVNPHVCRLLGICLT
STVQLITQLMPFGCLLDYVREHKDNIGSQYLLNWCVQIAKGMNYLEDRRLVHRDLAARN
VLVKTPQHVKITDFGLAKLLGAEEKEYHAEGGKVPIKWMALESILHRIYTHQSDVWSYGV
TVWELMTFGSKPYDGIPASEISSILEKGERLPQPPICTIDVYMIMVKCWMIDADSRPKFRE
LIIEFSKMARDPQRYLVIQGDERMHLPSPTDSNFYRALMDEEDMDDVVDADEYLIPQQGF
FSSPSTSRTPLLSSLSATSNNSTVACIDRNGLQSCPIKEDSFLQRYSSDPTGALTEDSIDD
TFLPVPEYINQSVPKRPAGSVQNPVYHNQPLNPAPSRDPHYQDPHSTAVGNPEYLNTVQ
PTCVNSTFDSPAHWAQKGSHQISLDNPDYQQDFFPKEAKPNGIFKGSTAENAEYLRVAP
QSSEFIGA
GST-EGFR #3 (SVD between D770 and N771)
MSPILGYWKIKGLVQPTRLLLEYLEEKYEEHLYERDEGDKWRNKKFELGLEFPNLPYYID
GDVKLTQSMAIIRYIADKHNMLGGCPKERAEISMLEGAVLDIRYGVSRIAYSKDFETLKVD
FLSKLPEMLKMFEDRLCHKTYLNGDHVTHPDFMLYDALDVVLYMDPMCLDAFPKLVCFK
KRIEAIPQIDKYLKSSKYIAWPLQGWQATFGGGDHPPKSDPITSLYKKAGSDYDIPTTTEN
LYFQGRRRHIVRKRTLRRLLQERELVEPLTPSGEAPNQALLRILKETEFKKIKVLGSGAFG
TVYKGLWIPEGEKVKIPVAIKELREATSPKANKEILDEAYVMASVDSVDNPHVCRLLGICLT
STVQLITQLMPFGCLLDYVREHKDNIGSQYLLNWCVQIAKGMNYLEDRRLVHRDLAARN
VLVKTPQHVKITDFGLAKLLGAEEKEYHAEGGKVPIKWMALESILHRIYTHQSDVWSYGV
TVWELMTFGSKPYDGIPASEISSILEKGERLPQPPICTIDVYMIMVKCWMIDADSRPKFRE
LIIEFSKMARDPQRYLVIQGDERMHLPSPTDSNFYRALMDEEDMDDVVDADEYLIPQQGF
FSSPSTSRTPLLSSLSATSNNSTVACIDRNGLQSCPIKEDSFLQRYSSDPTGALTEDSIDD
TFLPVPEYINQSVPKRPAGSVQNPVYHNQPLNPAPSRDPHYQDPHSTAVGNPEYLNTVQ
PTCVNSTFDSPAHWAQKGSHQISLDNPDYQQDFFPKEAKPNGIFKGSTAENAEYLRVAP
QSSEFIGA

### Generation of Recombinant Baculovirus:

In separate approaches each of the three transfer vectors was co-transfected in Sf9 cells with Baculovirus DNA (Flashbac Gold DNA, Oxford Expression Technologies) using Fugene HD (Roche). After 5 days the supernatant of the transfected cells containing the recombinant Baculovirus encoding the various EGFR proteins was used for further infection of Sf9 cells for virus amplification whereby the virus titer was monitored using qPCR.

### EGFR Expression in Sf9 Cells Using Bioreactor:

Sf9 cells cultured (Insect-xpress medium, Lonza, 27° C.) in a Wave-bioreactor with a disposable culture bag were infected at a cell density of 10⁶ cells/ml with one of the recombinant baculovirus stocks at a multiplicity of infection of 1 and incubated for 48 h. Subsequently the cells were harvested by centrifugation and the cell pellet frozen at ~80° C.

### Purification of the GST-EGFR Fusion Proteins:

Purification of the GST-EGFR fusion proteins was achieved by affinity chromatography using Glutathion Sepharose 4B matrix (GE Healthcare Life Sciences).

The pelleted cells (from 4 l cell culture) were resuspended in Lysis-Buffer (50 mM HEPES pH 7.4, 150 mM NaCl, 5% Glycerol, 1 mM MgCl2, 1 mM MnCl2, 0.5 mM Na3VO4) and lysed by a freeze-thaw cycle followed by an incubation on ice for 60 min. The supernatant was centrifuged at 4000×g for 30 min. at 4° C. The supernatant was than incubated with Glutathion Sepharose 4B matrix (in a glass bottle rotating for 16 h, at 4° C.) for binding of the GST EGFR fusion protein, rinsed with Wash-Buffer and finally the bound protein was eluted using Elusion-Buffer (Lysis Buffer plus 25 mM Glutathione) and shock frozen with liquid nitrogen.

### WT-EGFR Kinase Assay

Inhibitory activity of compounds of the present invention against wild-type Epidermal Growth Factor Receptor (EGFR) was quantified employing the TR-FRET based EGFR assay as described in the following paragraphs.

Recombinant fusion protein of N-terminal Glutathion-S-Transferase (GST) and a fragment of human EGFR (amino acids R669 to A1210), expressed in Sf9 insect cells and purified via affinity chromatography using Glutathion Sepharose as described above, was used as a kinase. As substrate for the kinase reaction the biotinylated peptide biotin-Ahx-AEEEEYFELVAKKK (C-terminus in amide form) was used, which can be purchased e.g. form the company Biosynthan GmbH (Berlin-Buch, Germany).

For the assay 50 nl of a 100 fold concentrated solution of the test compound in DMSO was pipetted into either a black low volume 384 well microtiter plate or a black 1536 well microtiter plate (both Greiner Bio-One, Frickenhausen, Germany), 2 μl of a solution of EGFR in aqueous assay buffer [50 mM Hepes pH 7.0, 10 mM MgCl2, 1 mM dithiothreitol, 0.5 mM EGTA, 0.3 mM activated sodium ortho-vanadate, 0.005% (w/v) bovine serum albumin, 0.005% (v/v) Tween-20] were added and the mixture was incubated for 15 min at 22° C. to allow pre binding of the test compounds to the enzyme before the start of the kinase reaction. Then the kinase reaction was started by the addition of 3 μL of a solution of adenosine tri phosphate (ATP, 3.33 mM=>final conc. in the 5 μL assay volume is 2 mM) and substrate (1.67 μM=>final conc. in the 5 μL assay volume is 1 μM) in assay buffer and the resulting mixture was incubated for a reaction time of 30 min at 22° C. The concentration of EGFR was adjusted depending of the activity of the enzyme lot and was chosen appropriate to have the assay in the linear range, typical concentration was 7.6 μg/μl. The reaction was stopped by the addition of 3 μl of a solution of HTRF detection reagents (83.3 nM streptavidine-XL665 [Cisbio Bioassays, Codolet, France] and 1.67 nM PT66-Tb-Cryptate, an terbium-cryptate labelled anti-phospho-tyrosine antibody from Cisbio Bioassays [instead of the PT66 Tb cryptate PT66 Eu Chelate from Perkin Elmer can also be used]) in an aqueous EDTA-solution (133.3 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Tb-Cryptate. Subsequently the amount of phosphorylated substrate was evaluated by measurement of the resonance energy transfer from the PT66-Tb-Cryptate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100-fold concentrated solutions in DMSO by serial dilutions, exact concentrations may vary depending pipettors used) in duplicate values for each concentration and IC₅₀ values were calculated using Genedata Screener™ software.

### Exon20-Mutant-EGFR (D770_N771insSVD) Kinase Assay

Inhibitory activity of compounds of the present invention against an Epidermal Growth Factor Receptor (EGFR) with an insertion of the amino acids sequence SVD between D770 and N771 was quantified employing the TR-FRET based kinase activity assay as described in the following paragraphs.

A recombinant fusion protein of N-terminal Glutathion-S-Transferase (GST) and a fragment of human EGFR variant (amino acids R669 to A1210 with insertion of the amino acids sequence SVD between D770 and N771 (“EGFR ins SVD”), expressed in Sf9 insect cells and purified via affinity chromatography using Glutathion Sepharose as described above, was used as a kinase. As substrate for the kinase reaction the biotinylated peptide biotin-Ahx-AEEEEYFELVAKKK (C-terminus in amide form) was used which can be purchased e.g. form the company Biosynthan GmbH (Berlin-Buch, Germany).

For the assay 50 nl of a 100-fold concentrated solution of the test compound in DMSO was pipetted into either a black low volume 384 well microtiter plate or a black 1536 well microtiter plate (both Greiner Bio-One, Frickenhausen, Germany), 2 μl of a solution of EGFR in aqueous assay buffer [50 mM Hepes pH 7.0, 10 mM MgCl2, 1 mM dithiothreitol, 0.5 mM EGTA, 0.3 mM activated sodium ortho-vanadate, 0.005% (w/v) bovine serum albumin, 0.005% (v/v) Tween-20] were added and the mixture was incubated for 15 min at 22° C. to allow pre binding of the test compounds to the enzyme before the start of the kinase reaction. Then the kinase reaction was started by the addition of 3 μL of a solution of adenosine tri phosphate (ATP, 3.33 mM=>final conc. in the 5 μL assay volume is 2 mM) and substrate (1.67 μM=>final conc. in the 5 μL assay volume is 1 μM) in assay buffer and the resulting mixture was incubated for a reaction time of 30 min at 22° C. The concentration of EGFR was adjusted depending of the activity of the enzyme lot and was chosen appropriate to have the assay in the linear range, typical concentration was 15 pg/μl. The reaction was stopped by the addition of 3 μl of a solution of HTRF detection reagents (83.3 nM streptavidine-XL665 [Cisbio Bioassays, Codolet, France] and 1.67 nM PT66-Tb-Cryptate, a terbium-cryptate labelled anti-phospho-tyrosine antibody from Cisbio Bioassays [instead of the PT66 Tb cryptate PT66 Eu Chelate from Perkin Elmer can also be used]) in an aqueous EDTA-solution (133.3 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Tb-Cryptate. Subsequently the amount of phosphorylated substrate was evaluated by measurement of the resonance energy transfer from the PT66-Tb-Cryptate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

### Exon20-Mutant-EGFR (V769_D770insASV) Kinase Assay

Inhibitory activity of compounds of the present invention against an Epidermal Growth Factor Receptor (EGFR) with an insertion of the amino acids sequence ASV between V769 and D770 was quantified employing the TR-FRET based kinase activity assay as described in the following paragraphs.

A recombinant fusion protein of N-terminal Glutathion-S-Transferase (GST) and a fragment of human EGFR variant (amino acids R669 to A1210 with insertion of the amino acids sequence ASV between V769 and D770; (“EGFR ins ASV”), expressed in Sf9 insect cells and purified via affinity chromatography using Glutathion Sepharose as described above, was used as kinase. As substrate for the kinase reaction the biotinylated peptide biotin-Ahx-AEEEEYFELVAKKK (C-terminus in amide form) was used which can be purchased e.g. form the company Biosynthan GmbH (Berlin-Buch, Germany).

For the assay 50 nl of a 100-fold concentrated solution of the test compound in DMSO was pipetted into either a black low volume 384 well microtiter plate or a black 1536 well microtiter plate (both Greiner Bio-One, Frickenhausen, Germany), 2 μl of a solution of EGFR in aqueous assay buffer [50 mM Hepes pH 7.0, 10 mM MgCl2, 1 mM dithiothreitol, 0.5 mM EGTA, 0.3 mM activated sodium ortho-vanadate, 0.005% (w/v) bovine serum albumin, 0.005% (v/v) Tween-20] were added and the mixture was incubated for 15 min at 22° C. to allow pre binding of the test compounds to the enzyme before the start of the kinase reaction. Then the kinase reaction was started by the addition of 3 μL of a solution of adenosine tri phosphate (ATP, 3.33 mM=>final conc. in the 5 μL assay volume is 2 mM) and substrate (1.67 μM=>final conc. in the 5 μL assay volume is 1 μM) in assay buffer and the resulting mixture was incubated for a reaction time of 30 min at 22° C. The concentration of EGFR was adjusted depending of the activity of the enzyme lot and was chosen appropriate to have the assay in the linear range, typical concentration was 2.5 pg/μl. The reaction was stopped by the addition of 3 μl of a solution of HTRF detection reagents (83.3 nM streptavidine-XL665 [Cisbio Bioassays, Codolet, France] and 1.67 nM PT66-Tb-Cryptate, an terbium-cryptate labelled anti-phospho-tyrosine antibody from Cisbio Bioassays [instead of the PT66 Tb cryptate PT66 Eu Chelate from Perkin Elmer can also be used]) in an aqueous EDTA-solution (133.3 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Tb-Cryptate. Subsequently the amount of phosphorylated substrate was evaluated by measurement of the resonance energy transfer from the PT66-Tb-Cryptate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100-fold concentrated solutions in DMSO by serial dilutions, exact concentrations may vary depending pipettors used) in duplicate values for each concentration and IC₅₀ values were calculated using Genedata Screener™ software. Table 2a and Table 2b show the results of the inhibition in mutant EGFR biochemical assay.

TABLE 2a

| | mutEGFR | mutEGFR |
|---|---|---|
| | (D770_N771insSVD) | (D770_N771insNPG, |
| | kinase assay | T790) kinase assay |
| Example | IC₅₀ | IC₅₀ |
| No. | [mol/l] | [mol/l] |
| 1 | 6.18E−10 | 9.53E−10 |
| 2 | <7.25E−11 | 1.46E−10 |
| | 1.05E−10 | |
| 3 | 1.39E−10 | 3.24E−10 |
| 4 | 1.45E−9 | 1.47E−9 |
| 5 | 2.19E−9 | 1.04E−9 |
| 6 | 3.26E−10 | 4.07E−10 |
| 7 | 2.09E−10 | 4.08E−10 |
| 8 | 3.93E−10 | 7.65E−10 |
| 9 | 6.45E−8 | 1.04E−7 |
| 10 | 8.49E−10 | 2.26E−9 |
| 11 | 2.96E−8 | 4.38E−8 |
| 12 | 3.71E−8 | 8.42E−8 |
| 13 | 8.07E−10 | 1.26E−9 |
| 14 | 4.06E−10 | 8.67E−10 |
| 15 | 7.29E−10 | 1.08E−9 |
| 16 | 5.52E−8 | 7.66E−8 |
| 17 | 5.57E−8 | 8.58E−8 |
| 18 | 4.51E−10 | 1.76E−9 |
| 19 | 2.59E−8 | 2.10E−7 |
| 20 | 3.54E−9 | 9.13E−9 |
| 21 | 1.17E−9 | 4.45E−9 |
| 22 | 5.12E−9 | 3.34E−9 |
| 23 | 2.08E−7 | 8.04E−7 |
| 24 | 1.15E−6 | 1.46E−5 |
| | | >2.00E−5 |
| 25 | 6.00E−10 | 2.12E−9 |
| 26 | 1.26E−9 | 3.30E−9 |
| 27 | 3.77E−9 | 3.02E−9 |
| 28 | 7.02E−8 | 3.52E−8 |
| 29 | 1.19E−9 | 1.54E−9 |
| 30 | 7.25E−9 | 2.37E−8 |
| 31 | 4.05E−9 | 1.88E−8 |
| 32 | 1.31E−9 | 3.89E−9 |
| 33 | 8.35E−9 | 6.70E−8 |
| 34 | 1.37E−9 | 4.32E−9 |
| 35 | 1.36E−9 | 2.94E−9 |
| 36 | 3.21E−10 | 1.13E−9 |
| 37 | 2.58E−10 | 6.58E−10 |
| 38 | 5.54E−10 | 4.89E−10 |
| 39 | 3.76E−8 | 1.83E−8 |
| 40 | 9.82E−11 | 1.48E−10 |
| 41 | 1.09E−9 | 3.22E−9 |
| 42 | 1.01E−9 | 6.31E−9 |
| 43 | 3.16E−9 | 1.85E−8 |
| 44 | 9.89E−10 | 2.80E−9 |
| 45 | 9.73E−7 | 1.58E−7 |
| 46 | 7.70E−7 | 5.55E−7 |
| 47 | 8.64E−7 | 6.66E−7 |
| 48 | 2.42E−7 | 1.27E−6 |
| 49 | 1.12E−7 | 1.21E−7 |
| 50 | 8.50E−9 | 1.35E−7 |
| 51 | 6.64E−9 | 2.57E−8 |
| 52 | 6.05E−9 | 2.27E−8 |
| 53 | 9.97E−10 | 7.05E−9 |
| 54 | 8.90E−8 | 3.47E−6 |
| 55 | 3.50E−7 | 2.54E−6 |
| 56 | 2.81E−7 | 6.70E−7 |
| 57 | 1.65E−7 | 2.56E−7 |
| 58 | 4.46E−7 | 1.30E−6 |
| 59 | 8.13E−7 | 9.56E−7 |
| 60 | 9.39E−7 | 1.41E−6 |
| 61 | 2.38E−6 | 3.98E−7 |
| 62 | 2.95E−8 | 8.76E−8 |
| 63 | 2.64E−9 | 9.00E−9 |
| 64 | 2.68E−7 | 6.98E−7 |
| 65 | 3.82E−10 | 9.52E−10 |
| 66 | 8.72E−8 | 2.22E−7 |
| 67 | 1.22E−9 | 3.30E−9 |
| 68 | 5.33E−9 | 1.19E−8 |
| 69 | 1.73E−9 | 4.78E−9 |
| 70 | 2.61E−8 | 9.02E−8 |
| 71 | 9.07E−10 | 3.09E−9 |
| 72 | 2.11E−10 | 3.00E−10 |
| 73 | 1.09E−9 | 5.10E−9 |
| 74 | 1.83E−10 | 8.07E−10 |
| 75 | 1.97E−10 | 9.95E−10 |
| 76 | 3.03E−8 | 7.73E−8 |
| 77 | 3.47E−10 | 1.60E−9 |
| 78 | 3.86E−9 | 1.54E−8 |
| 79 | 1.47E−9 | 2.51E−9 |
| 80 | 1.94E−8 | |
| 81 | 4.15E−9 | |
| 82 | 2.96E−8 | |
| 83 | 9.76E−10 | 3.53E−9 |
| 84 | 1.34E−8 | 5.06E−8 |
| 85 | 7.31E−10 | 2.43E−9 |
| 86 | 1.22E−7 | 2.37E−7 |
| 87 | 5.31E−8 | 8.25E−8 |
| 88 | 9.27E−9 | 1.58E−8 |
| 89 | 6.06E−9 | 8.70E−9 |
| 90 | 2.08E−9 | 5.34E−9 |
| 91 | 2.63E−8 | 5.28E−8 |
| 92 | 2.46E−8 | 5.36E−8 |
| 93 | 3.63E−6 | 8.79E−7 |
| 94 | 2.13E−7 | 4.03E−7 |
| 95 | 4.06E−8 | 9.95E−8 |
| 96 | 2.03E−6 | 7.96E−6 |
| 97 | 6.26E−7 | 3.80E−6 |
| 98 | 1.04E−8 | 1.16E−8 |
| 99 | 5.72E−9 | 5.71E−8 |
| 100 | 1.96E−8 | 3.57E−9 |
| 101 | 1.68E−8 | 3.50E−9 |
| 102 | 4.77E−9 | 2.28E−8 |
| 103 | 3.70E−9 | 2.52E−8 |
| 104 | 1.66E−9 | 9.09E−9 |
| 105 | 1.48E−9 | 1.57E−9 |
| 106 | 5.28E−10 | 5.46E−10 |
| 107 | 8.44E−10 | 5.64E−10 |
| 108 | 3.51E−10 | 4.25E−10 |
| 109 | 1.11E−10 | 1.49E−10 |
| 110 | 7.50E−10 | 1.93E−9 |
| 111 | 1.30E−10 | 2.59E−10 |
| 112 | 1.96E−10 | 4.48E−10 |
| 113 | 1.76E−9 | 3.91E−9 |
| 114 | 1.44E−10 | 2.18E−10 |
| 115 | 2.20E−10 | 9.49E−10 |
| 116 | 8.38E−9 | 1.15E−8 |
| 117 | 3.84E−10 | 1.86E−9 |
| 118 | 8.55E−9 | 4.90E−9 |
| 119 | 2.32E−8 | 7.03E−9 |
| 120 | 5.05E−8 | 5.03E−8 |
| 121 | 1.88E−9 | |
| 122 | 3.69E−10 | 8.03E−10 |
| 123 | 1.19E−9 | 1.95E−9 |
| 124 | 8.59E−10 | 2.08E−9 |
| 125 | 1.12E−9 | |
| 126 | 1.24E−8 | 4.52E−9 |
| 127 | 2.54E−8 | 5.65E−9 |
| 128 | 3.62E−9 | 1.38E−8 |
| 129 | 2.97E−9 | 1.60E−8 |
| 130 | 6.69E−9 | 4.55E−8 |
| 131 | 1.79E−9 | 8.01E−9 |
| 132 | 9.57E−10 | 1.31E−9 |
| 133 | 3.01E−9 | 3.83E−9 |
| 134 | 2.10E−9 | 9.18E−9 |
| 135 | 4.71E−10 | 1.80E−9 |
| 136 | 1.10E−9 | 4.74E−9 |
| 137 | 1.60E−9 | 1.08E−8 |
| 138 | 9.81E−10 | 2.93E−9 |
| 139 | 8.41E−10 | 1.38E−9 |
| 140 | 8.12E−10 | 3.08E−9 |
| 141 | 3.24E−8 | 1.97E−8 |
| 142 | 7.39E−10 | 2.83E−9 |
| 143 | 1.15E−8 | 7.19E−8 |
| 144 | 8.69E−9 | 3.10E−8 |
| 145 | 9.01E−9 | 6.04E−8 |
| 146 | 6.80E−7 | 1.17E−6 |
| 147 | 3.60E−8 | 2.01E−7 |
| 148 | 1.91E−9 | 9.10E−9 |
| 149 | 1.54E−9 | 2.59E−9 |
| 150 | 7.64E−10 | 1.05E−9 |
| 151 | 3.20E−9 | 1.21E−8 |
| 152 | 1.05E−9 | 4.55E−9 |
| 153 | 2.29E−9 | 9.91E−9 |
| 154 | 3.48E−9 | 2.75E−8 |
| 155 | 6.43E−9 | 5.24E−9 |
| 156 | 1.45E−9 | 4.64E−9 |
| 157 | 1.00E−9 | 1.77E−9 |
| 158 | 5.01E−9 | 1.64E−8 |
| 159 | 8.31E−8 | 3.87E−8 |
| 160 | 1.48E−9 | 6.58E−9 |
| 161 | 9.29E−8 | 6.29E−7 |
| 162 | 5.07E−10 | 2.32E−9 |
| 163 | 1.60E−10 | 6.19E−10 |
| 164 | 2.99E−9 | 9.37E−9 |
| 165 | 6.31E−10 | 2.43E−9 |
| 166 | 1.69E−10 | 3.38E−10 |
| 167 | 2.56E−10 | 1.02E−9 |
| 168 | 2.45E−10 | 6.92E−10 |
| 169 | 1.80E−10 | 3.85E−10 |
| 170 | 3.80E−10 | |
| 171 | 1.36E−10 | 4.06E−10 |
| 172 | 2.03E−10 | 1.21E−9 |
| 173 | 1.57E−7 | 2.55E−8 |
| 174 | 1.76E−7 | 2.13E−6 |
| 175 | 9.76E−8 | 8.04E−8 |
| 176 | 1.66E−7 | |
| 177 | 4.32E−8 | 1.78E−8 |
| 178 | 2.07E−7 | |
| 179 | 4.38E−7 | 3.60E−7 |
| 180 | 1.58E−7 | 6.15E−8 |
| 181 | 5.96E−8 | |
| 182 | 8.61E−7 | 1.08E−6 |
| 183 | 9.68E−8 | 2.17E−7 |
| 184 | 1.55E−7 | 5.28E−8 |
| 185 | 2.33E−7 | |
| 186 | 2.10E−7 | 1.29E−7 |
| 187 | 3.00E−8 | |
| 188 | 1.55E−7 | 1.12E−8 |
| 189 | 1.02E−9 | 1.74E−9 |
| 190 | 1.03E−9 | 3.03E−9 |
| 191 | 6.26E−10 | 1.26E−9 |
| 192 | <7.25E−11 | 9.16E−11 |
| 193 | 8.31E−10 | 2.85E−9 |
| 194 | 1.44E−10 | 3.99E−10 |
| 195 | 8.25E−11 | 2.19E−10 |
| 196 | 1.83E−10 | 4.21E−10 |
| 197 | 1.45E−9 | 8.01E−9 |
| 198 | 4.76E−7 | 1.07E−6 |
| 199 | 6.72E−10 | 2.05E−9 |
| 200 | 1.61E−10 | 3.17E−10 |
| 201 | 1.67E−9 | 3.42E−9 |
| 202 | 5.74E−8 | 2.25E−7 |
| 203 | 4.09E−10 | 1.42E−9 |
| 204 | 4.61E−9 | 1.72E−8 |
| 205 | 1.37E−7 | 1.44E−7 |
| 206 | 5.53E−8 | 8.29E−8 |
| 207 | 6.44E−10 | 2.22E−9 |
| 208 | 1.40E−10 | 3.65E−10 |
| 209 | 2.26E−10 | 6.94E−10 |
| 210 | 2.59E−8 | 6.04E−8 |
| 211 | 1.25E−8 | 4.05E−8 |
| 212 | 5.61E−9 | 1.01E−8 |
| 213 | 9.02E−10 | 3.27E−9 |
| 214 | 2.72E−10 | 6.54E−10 |
| 215 | 5.13E−10 | 1.20E−9 |
| 216 | 2.50E−8 | 1.64E−7 |
| 217 | 6.20E−9 | 5.98E−8 |
| 218 | 6.99E−9 | 1.64E−8 |
| 219 | 7.15E−10 | 1.21E−9 |
| 220 | 4.15E−9 | 9.66E−9 |
| 221 | 3.69E−9 | 1.53E−8 |
| 222 | 3.76E−9 | 1.30E−8 |
| 223 | 3.36E−10 | 1.25E−9 |
| 224 | 2.38E−9 | 7.59E−9 |
| 225 | 1.01E−8 | 3.66E−8 |
| 226 | 1.40E−8 | 2.33E−8 |
| 227 | 1.20E−10 | 3.94E−10 |
| 228 | 7.24E−10 | 2.20E−9 |
| 229 | 1.88E−10 | 3.51E−10 |
| 230 | 4.16E−10 | 1.04E−9 |
| 231 | 5.02E−10 | 1.10E−9 |
| 232 | 1.16E−10 | 1.64E−10 |
| 233 | 2.58E−10 | 4.67E−10 |
| 234 | 1.70E−10 | 3.31E−10 |
| 235 | 1.80E−10 | 5.92E−10 |
| 236 | 1.81E−10 | 2.89E−10 |
| 237 | 2.94E−9 | 9.96E−9 |
| 238 | 1.57E−9 | 2.21E−9 |
| 239 | 1.39E−9 | 4.74E−9 |
| 240 | 4.37E−10 | 1.50E−9 |
| 241 | 3.88E−9 | 1.23E−8 |
| 242 | 4.45E−10 | 8.39E−10 |
| 243 | 3.95E−10 | 6.56E−10 |
| 244 | 7.49E−10 | 1.99E−9 |
| 245 | 1.13E−10 | 2.62E−10 |
| 246 | 1.31E−10 | 2.03E−10 |
| 247 | 6.99E−9 | 9.33E−7 |
| 248 | 7.81E−8 | 6.04E−7 |
| 249 | 3.72E−7 | 5.75E−8 |
| 250 | 4.35E−7 | 2.16E−7 |
| 251 | 4.09E−8 | 7.23E−9 |
| 252 | 1.17E−8 | 2.84E−6 |
| 253 | 2.47E−7 | 3.95E−6 |
| 254 | 3.60E−8 | 4.37E−7 |
| 255 | 3.18E−6 | 9.61E−6 |
| 256 | 7.49E−6 | >1.63E−6 |
| 257 | 6.97E−8 | 2.08E−8 |
| 258 | 1.00E−7 | 1.07E−6 |
| 259 | 6.83E−7 | 1.33E−5 |
| 260 | 8.29E−8 | 1.28E−6 |
| 261 | 6.50E−9 | 7.30E−9 |
| 262 | 1.62E−6 | 3.96E−7 |
| 263 | 1.12E−7 | 2.46E−8 |
| 264 | 7.65E−7 | 1.13E−6 |
| 265 | 4.04E−6 | 6.30E−6 |
| 266 | 4.62E−7 | 3.58E−6 |
| 267 | 2.49E−8 | 2.17E−8 |
| 268 | 9.40E−7 | 4.46E−7 |
| 269 | 1.38E−7 | 3.82E−8 |
| 270 | 2.16E−10 | 1.03E−9 |
| 271 | 1.32E−10 | 3.51E−10 |
| 272 | 2.35E−9 | 1.04E−8 |
| 273 | 3.94E−9 | 2.16E−8 |
| 274 | 1.35E−9 | 3.11E−9 |
| 275 | 4.76E−10 | 1.22E−9 |
| 276 | 9.09E−11 | 1.57E−10 |
| 277 | <7.25E−11 | 1.53E−10 |
| | 9.55E−11 | |
| | 1.11E−10″ | |
| 278 | 2.04E−10 | 5.51E−10 |
| 279 | 5.06E−8 | 6.22E−8 |
| 280 | 8.13E−11 | 1.51E−10 |
| 281 | 1.70E−10 | 3.66E−10 |
| 282 | 1.17E−10 | 2.82E−10 |
| 283 | 2.49E−7 | 3.03E−8 |
| 284 | 1.38E−6 | 1.74E−7 |
| 285 | 2.32E−10 | 6.44E−10 |
| 286 | 7.10E−8 | 9.57E−8 |
| 287 | 2.20E−10 | 4.15E−10 |
| 288 | 3.44E−7 | 5.38E−8 |
| 289 | 6.74E−6 | 3.48E−7 |
| 290 | 1.18E−6 | |
| 291 | 1.11E−9 | |
| 293 | 5.03E−8 | 9.60E−8 |
| 292 | 1.29E−10 | 3.40E−10 |

TABLE 2b
(From US Priority Patent Application)

| | mutEGFR | |
|---|---|---|
| | (D770_N771insSVD) | mutEGFR (D770_N771insNPG, |
| | kinase assay | T790) kinase assay |
| Example | IC₅₀ | IC₅₀ |
| No. | [mol/l] | [mol/l] |
| 1 | 6.18E−10 | 9.53E−10 |
| 2 | <7.25E−11 | 1.46E−10 |
| | 1.05E−10 | |
| 3 | 1.39E−10 | 3.24E−10 |
| 4 | 1.45E−9 | 1.47E−9 |
| 5 | 2.19E−9 | 1.04E−9 |
| 6 | 3.26E−10 | 4.07E−10 |
| 7 | 2.09E−10 | 4.08E−10 |
| 8 | 3.93E−10 | 7.65E−10 |
| 9 | 6.45E−8 | 1.04E−7 |
| 10 | 8.49E−10 | 2.26E−9 |
| 11 | 2.96E−8 | 4.38E−8 |
| 12 | 3.71E−8 | 8.42E−8 |
| 13 | 8.07E−10 | 1.26E−9 |
| 14 | 4.06E−10 | 8.67E−10 |
| 15 | 7.29E−10 | 1.08E−9 |
| 16 | 5.52E−8 | 7.66E−8 |
| 17 | 5.57E−8 | 8.58E−8 |
| 18 | 4.51E−10 | 1.76E−9 |
| 19 | 2.59E−8 | |
| 20 | 3.54E−9 | |
| 21 | 1.17E−9 | |
| 22 | 5.12E−9 | |
| 23 | 2.08E−7 | |
| 24 | 1.15E−6 | |

Cellular Data Description (WT, insSVD, insSVD T790M) 293T cells from ATCC were transfected with pBABEpuro expression constructs for WT EGFR or EGFR-insSVD, or EGFR-insSVD T790M, and pCL-Eco packaging vector using Fugene-6 transfection reagent from Promega. Plates were incubated at at 37° C. for 48 h. Retrovirus was harvested by filtering the media supernatant through a 0.45 μm filter.

Ba/F3 cells purchased from DSMZ were grown in RPMI+10% FBS+10 ng/ml IL-3 and infected with filtered retroviral supernatant at a 1:2 dilution. Polybrene was added to a concentration of 8 μg/mL, plates were spun for 90 min, and incubated for 16 h at 37° C. 2 μg/mL puromycin was added to the infected cells 24 h after infection and cells were continually grown in the presence of puromycin and 10 ng/ml IL-3. Following stably expressing Ba/F3 cell lines were generated: Ba/F3-EGFR-WT, Ba/F3-EGFR-insSVD, Ba/F3-EGFR-insSVD T790M, (Ba/F3-vector-control).

For cell survival assays, Ba/F3 cells were grown to a density of 1-2 million cells per mL, spun down and resuspended in media without IL-3, and replated at a concentration of 200,000-500,000 cells per mL. The cells ectopically expressing WT EGFR, EGFR-insSVD, or EGFR-insSVD T790M were plated with 10 ng/ml Millipore Culture grade EGF. The cells ectopically expressing pBABEpuro empty vector were plated with 10 ng/mL IL-3.

2 days later, cells were plated in 50 UL in a 384 well plate at a concentration of 4000 cells per well for cells assayed in the absence of IL-3 and 2000 cells per well for cells assayed in the presence of IL-3. 100 nL of compound was added to each well using a 100 nL pin head, and plates were incubated at 37° C. for 48 h.

Cell viability was measured by adding 20 μL of Cell Titer-Glo Luminescent Cell Viability Reagent diluted 1:3 in PBS. Plates were sealed with Perkin Elmer Top-Seal, inverted several times to mix, and immediately centrifuged at 1000 rpm for 2 min. Plates were incubated in low light conditions for 8-10 min and luminescence was measured. The IC₅₀ values for the examples are shown in Table 3a and Table 3b.

TABLE 3a

| | | BA/F3 (EGFR D770_N771 |
|---|---|---|
| | BA/F3 (insSVD) | insSVD T790M) |
| Example No. | IC₅₀ [mol/l] | IC₅₀ [mol/l] |
| 1 | 3.46E−8 | 1.19E−7 |
| 2 | 2.06E−8 | |
| 3 | 1.32E−8 | 1.08E−7 |
| 4 | 5.05E−8 | |
| 5 | 8.05E−8 | 1.14E−7 |
| 6 | 2.23E−8 | 2.07E−7 |
| 7 | 1.24E−8 | 6.01E−8 |
| 8 | 2.04E−8 | |
| 9 | 2.04E−6 | |
| 10 | 4.65E−8 | 3.46E−7 |
| 11 | 1.56E−6 | |
| 12 | 3.59E−7 | |
| 13 | 9.09E−7 | |
| 14 | 7.60E−7 | 9.83E−7 |
| 15 | 8.69E−8 | |
| 16 | 7.74E−7 | |
| 17 | 1.15E−6 | |
| 18 | 1.43E−7 | 9.98E−6 |
| 19 | 8.23E−7 | |
| 20 | 6.17E−7 | |
| 21 | 1.01E−7 | |
| 22 | 6.20E−8 | |
| 23 | 3.85E−6 | |
| 24 | 9.98E−6 | |
| 25 | 6.23E−8 | |
| 26 | 7.03E−8 | |
| 27 | 6.79E−7 | |
| 28 | 3.98E−6 | |
| 29 | 4.22E−7 | |
| 30 | 1.08E−6 | |
| 31 | 8.74E−7 | |
| 32 | 1.07E−6 | |
| 33 | 1.84E−6 | |
| 34 | 1.98E−7 | |
| 35 | 8.02E−7 | |
| 36 | 1.79E−8 | 1.26E−7 |
| 37 | 7.14E−9 | 1.04E−7 |
| 38 | 3.21E−8 | |
| 39 | 6.22E−7 | |
| 40 | 1.49E−8 | 5.60E−8 |
| 41 | 3.72E−8 | |
| 42 | 5.36E−8 | |
| 43 | 1.31E−7 | |
| 44 | 3.90E−8 | |
| 45 | 2.18E−7 | |
| 46 | 1.70E−7 | |
| 47 | 2.31E−7 | |
| 48 | 9.05E−8 | |
| 49 | 7.17E−8 | |
| 50 | 1.56E−7 | |
| 51 | 5.47E−8 | |
| 52 | 3.55E−8 | |
| 53 | 4.42E−8 | |
| 54 | 4.98E−7 | |
| 55 | 7.44E−7 | |
| 56 | 1.24E−7 | |
| 57 | 8.57E−8 | |
| 58 | 1.38E−7 | |
| 59 | 4.88E−7 | |
| 60 | 6.47E−7 | |
| 61 | 9.22E−7 | |
| 62 | 3.90E−7 | |
| 63 | 2.21E−7 | |
| 64 | 5.05E−6 | |
| 65 | 1.53E−8 | 1.01E−6 |
| 66 | 1.75E−6 | |
| 67 | 8.34E−8 | |
| 68 | 6.02E−8 | |
| 69 | 1.94E−7 | |
| 70 | 8.62E−7 | |
| 71 | 1.20E−7 | |
| 72 | 1.79E−8 | |
| 73 | 6.10E−8 | |
| 74 | 3.40E−8 | |
| 75 | 1.35E−7 | |
| 76 | 6.66E−7 | |
| 77 | 2.59E−8 | |
| 78 | 1.15E−7 | |
| 79 | 6.29E−8 | |
| 80 | 1.37E−6 | |
| 81 | 1.34E−7 | |
| 82 | 7.39E−7 | |
| 83 | 1.32E−7 | |
| 84 | 1.23E−6 | |
| 85 | 3.46E−8 | |
| 86 | 9.71E−7 | |
| 87 | 1.06E−6 | |
| 88 | 3.79E−6 | |
| 89 | 5.06E−6 | |
| 90 | 1.66E−7 | |
| 91 | 1.41E−6 | |
| 92 | 1.25E−7 | |
| 93 | 2.95E−6 | |
| 94 | 2.76E−6 | |
| 95 | 1.21E−6 | |
| 96 | 7.93E−6 | |
| 97 | 4.36E−6 | |
| 98 | 2.09E−7 | |
| 99 | 3.37E−7 | |
| 100 | 4.20E−7 | |
| 101 | 4.46E−8 | |
| 102 | 4.98E−8 | |
| 103 | 1.22E−7 | |
| 104 | 3.20E−8 | |
| 105 | 2.33E−7 | |
| 106 | 2.14E−7 | |
| 107 | 4.14E−8 | |
| 108 | 3.33E−8 | |
| 109 | 4.20E−8 | |
| 110 | 3.32E−8 | |
| 111 | 2.75E−8 | |
| 112 | 2.03E−8 | |
| 113 | 5.55E−8 | |
| 114 | 1.37E−8 | |
| 115 | 4.22E−8 | |
| 116 | 6.35E−7 | |
| 117 | 5.45E−8 | |
| 118 | 1.83E−7 | |
| 119 | 1.53E−7 | |
| 120 | 9.33E−7 | |
| 121 | 2.13E−7 | |
| 122 | 1.38E−8 | |
| 123 | 2.37E−8 | |
| 124 | 3.04E−8 | |
| 125 | 4.55E−8 | |
| 126 | 8.65E−8 | |
| 127 | 3.90E−8 | |
| 128 | 1.39E−7 | |
| 129 | 1.06E−7 | |
| 130 | 9.80E−7 | |
| 131 | 6.13E−8 | |
| 132 | 4.42E−8 | |
| 133 | 1.72E−7 | |
| 134 | 6.51E−8 | |
| 135 | 2.77E−8 | |
| 136 | 6.83E−8 | |
| 137 | 1.44E−7 | |
| 138 | 2.32E−8 | |
| 139 | 2.59E−8 | |
| 140 | 5.14E−8 | |
| 141 | 1.77E−7 | |
| 142 | 5.30E−8 | |
| 143 | 6.73E−7 | |
| 144 | 1.49E−6 | |
| 145 | 6.07E−7 | |
| 146 | 4.68E−6 | |
| 147 | 2.94E−6 | |
| 148 | 3.87E−7 | |
| 149 | 2.00E−7 | |
| 150 | 5.43E−8 | |
| 151 | 4.05E−7 | |
| 152 | 2.57E−7 | |
| 153 | 5.57E−7 | |
| 154 | 2.85E−6 | |
| 155 | 1.05E−6 | |
| 156 | 9.82E−8 | |
| 157 | 1.17E−7 | |
| 158 | 4.82E−7 | |
| 159 | 7.64E−7 | |
| 160 | 9.73E−7 | |
| 161 | 1.99E−6 | |
| 162 | 4.38E−8 | |
| 163 | 4.74E−8 | |
| 164 | 1.55E−7 | |
| 165 | 1.07E−7 | |
| 166 | 7.81E−8 | |
| 167 | 3.52E−8 | |
| 168 | 6.37E−8 | |
| 169 | 6.69E−8 | |
| 170 | 1.90E−7 | |
| 171 | 3.37E−8 | |
| 172 | 4.32E−8 | |
| 173 | 6.28E−8 | |
| 174 | 1.60E−7 | |
| 175 | 6.35E−8 | |
| 176 | 6.27E−8 | |
| 177 | 5.87E−8 | |
| 178 | 5.86E−8 | |
| 179 | 2.65E−7 | |
| 180 | 1.12E−7 | |
| 181 | 6.41E−8 | |
| 182 | 5.31E−7 | |
| 183 | 6.56E−8 | |
| 184 | 5.66E−8 | |
| 185 | 6.88E−8 | |
| 186 | 5.57E−7 | |
| 187 | | |
| 188 | 1.49E−7 | |
| 189 | 2.23E−8 | |
| 190 | 1.72E−7 | |
| 191 | 3.29E−8 | |
| 192 | 1.48E−7 | 3.97E−8 |
| 193 | 4.56E−8 | |
| 194 | 1.53E−8 | |
| 195 | 8.90E−9 | 4.33E−8 |
| 196 | 4.04E−8 | |
| 197 | 9.95E−7 | |
| 198 | 6.39E−6 | |
| 199 | 1.32E−7 | |
| 200 | 6.90E−8 | |
| 201 | 4.23E−7 | |
| 202 | 9.98E−6 | |
| 203 | 1.74E−8 | 1.08E−7 |
| 204 | 1.34E−7 | |
| 205 | 8.24E−7 | |
| 206 | 6.00E−7 | |
| 207 | 2.46E−8 | |
| 208 | 1.58E−8 | |
| 209 | 8.15E−9 | |
| 210 | 5.51E−7 | |
| 211 | 3.78E−7 | |
| 212 | 2.96E−7 | |
| 213 | 1.12E−7 | |
| 214 | 6.90E−8 | |
| 215 | 4.17E−8 | |
| 216 | 4.18E−7 | |
| 217 | 2.77E−7 | |
| 218 | 3.67E−7 | |
| 219 | 3.08E−7 | |
| 220 | 5.88E−6 | |
| 221 | 2.51E−6 | |
| 222 | 6.58E−7 | |
| 223 | 1.80E−8 | |
| 224 | 3.38E−7 | |
| 225 | 3.86E−7 | |
| 226 | 1.34E−7 | |
| 227 | 5.94E−9 | |
| 228 | 4.46E−8 | |
| 229 | 1.71E−8 | |
| 230 | 1.56E−8 | |
| 231 | 6.59E−8 | |
| 232 | 1.55E−8 | |
| 233 | 4.87E−8 | |
| 234 | 1.22E−7 | |
| 235 | 2.41E−8 | |
| 236 | 2.56E−7 | |
| 237 | 1.26E−7 | |
| 238 | 6.87E−8 | |
| 239 | 3.87E−8 | |
| 240 | 5.40E−8 | |
| 241 | 7.71E−8 | |
| 242 | 4.82E−8 | |
| 243 | 9.78E−8 | |
| 244 | 1.68E−7 | |
| 245 | 1.19E−8 | |
| 246 | 3.37E−8 | |
| 247 | 1.34E−7 | |
| 248 | 5.94E−8 | |
| 249 | 9.87E−8 | |
| 250 | 7.51E−8 | |
| 251 | 2.43E−8 | |
| 252 | 1.80E−7 | |
| 253 | 4.90E−7 | |
| 254 | 3.99E−8 | |
| 255 | 2.96E−7 | |
| 256 | 1.58E−7 | |
| 257 | 3.07E−8 | |
| 258 | 8.26E−8 | |
| 259 | 2.96E−7 | |
| 260 | 1.01E−7 | |
| 261 | 1.11E−7 | |
| 262 | 4.52E−7 | |
| 263 | 8.10E−8 | |
| 264 | 1.28E−7 | |
| 265 | 1.72E−7 | |
| 266 | 1.21E−7 | |
| 267 | 2.57E−7 | |
| 268 | 2.79E−7 | |
| 269 | 1.28E−7 | |
| 270 | 1.15E−8 | |
| 271 | 2.25E−7 | |
| 272 | 8.78E−8 | |
| 273 | 5.31E−7 | |
| 274 | 3.41E−8 | |
| 275 | 1.68E−8 | |
| 276 | 6.22E−9 | |
| 277 | 2.77E−9 | |
| 278 | 9.07E−9 | |
| 279 | 4.29E−7 | |
| 280 | 6.82E−9 | |
| 281 | 9.77E−9 | |
| 282 | 6.15E−9 | |
| 283 | 3.75E−8 | |
| 284 | 5.30E−7 | |
| 285 | 4.20E−8 | |
| 286 | 8.43E−7 | |
| 287 | 3.73E−8 | |
| 288 | 1.81E−7 | |
| 289 | 9.10E−7 | |
| 290 | 3.43E−6 | |
| 291 | 5.30E−8 | |
| 293 | 3.80E−7 | 9.98E−6 |
| 292 | 4.08E−9 | |

TABLE 3b
(From US Priority Patent Application)

| | | BA/F3 (EGFR D770_N771 |
|---|---|---|
| | BA/F3 (insSVD) | insSVD T790M) |
| Example No. | IC₅₀ [mol/l] | IC₅₀ [mol/l] |
| 1 | 3.46E−8 | 1.19E−7 |
| 2 | 2.06E−8 | |
| 3 | 1.32E−8 | 1.08E−7 |
| 4 | 5.05E−8 | |
| 5 | 8.05E−8 | 1.14E−7 |
| 6 | 2.23E−8 | 2.07E−7 |
| 7 | 1.24E−8 | 6.01E−8 |
| 8 | 2.04E−8 | |
| 9 | 2.04E−6 | |
| 10 | 4.65E−8 | 3.46E−7 |
| 11 | 1.56E−6 | |
| 12 | 3.59E−7 | |
| 13 | 9.09E−7 | |
| 14 | 7.60E−7 | 9.83E−7 |
| 15 | 8.69E−8 | |
| 16 | 7.74E−7 | |
| 17 | 1.15E−6 | |
| 18 | 1.43E−7 | 2.17E−10 |
| 19 | 8.23E−7 | |
| 20 | 6.17E−7 | |
| 21 | 1.01E−7 | |
| 22 | 6.20E−8 | |
| 23 | | |
| 24 | | |

### Cellular Data Description (L858R, E746_A750del, L858R T790M, E746_A750del T790M)

293T cells from ATCC were transfected with pBABEpuro expression constructs for EGFR-L858R, EGFR-E746_A750del, EGFR-L858R T790M, or EGFR-E746_A750del T790M, and pCL-Eco packaging vector using Fugene-6 transfection reagent from Promega. Plates were incubated at at 37° C. for 48 h. Retrovirus was harvested by filtering the media supernatant through a 0.45 μm filter.

Ba/F3 cells purchased from DSMZ were grown in RPMI+10% FBS+10 ng/ml IL-3 and infected with filtered retroviral supernatant at a 1:2 dilution. Polybrene was added to a concentration of 8 μg/mL, plates were spun for 90 min, and incubated for 16 h at 37° C. 2 μg/mL puromycin was added to the infected cells 24 h after infection and cells were continually grown in the presence of puromycin and 10 ng/ml IL-3. Following stably expressing Ba/F3 cell lines were generated: Ba/F3-EGFR-L858R, Ba/F3-EGFR-E746_A750del, Ba/F3-EGFR-L858R T790M, or Ba/F3-EGFR-E746_A750del T790M.

For cell survival assays, Ba/F3 cells were grown to a density of 1-2 million cells per mL, spun down and resuspended in media without IL-3, and replated at a concentration 200,000-500,000 cells per mL. The cells ectopically expressing EGFR-L858R, EGFR-E746_A750del, EGFR-L858R T790M, or EGFR-E746_A750del T790M were plated with 10 ng/ml Millipore Culture grade EGF. The cells ectopically expressing pBABEpuro empty vector were plated with 10 ng/mL IL-3.

2 days later, cells were plated in 50 μL in a 384 well plate at a concentration of 4000 cells per well for cells assayed in the absence of IL-3 and 2000 cells per well for cells assayed in the presence of IL-3. 100 nL of compound was added to each well using a 100 nL pin head, and plates were incubated at 37° C. for 48 h.

Cell viability was measured by adding 20 μL of Cell Titer-Glo Luminescent Cell Viability Reagent diluted 1:3 in PBS. Plates were sealed with Perkin Elmer Top-Seal, inverted several times to mix, and immediately centrifuged at 1000 rpm for 2 min. Plates were incubated in low light conditions for 8-10 min and luminescence was measured.

In contrast to the claimed compounds of this invention the compounds claimed in WO2021190616 and WO2021190615 do not show the advantageous combined properties described above. This is shown in Table 4.

TABLE 4

| | mutEGFR | |
|---|---|---|
| | (D770_N771insSVD) | |
| | kinase assay | BA/F3 (insSVD) |
| WO2021190616 | IC₅₀ | IC₅₀ |
| Example No. | [mol/l] | [mol/l] |
| 2-27 | >2.0E−5 | >9.98E−6 |
| 2-14 | >2.0E−5 | >9.98E−6 |

### P-gp Permeation Assay

P-glycoprotein (P-gp, MDR1) substrate characteristics of test compounds were investigated by assessing their permeability across a monolayer of P-gp overexpressing LLC-PK1 cells (L-MDR1 cells). L-MDR1 cells (purchased from the Dutch Cancer Institute, Amsterdam, the Netherlands) were maintained in cell culture media (Medium 199 supplemented with 2 mM L-glutamine, 10% fetal calf serum, 10 mg/500 ml of streptomycin, and 10,000 IU/500 ml of penicillin) at 37° C. in a humidified incubator with 5% CO2.

For cultivation, L-MDR1 cells were seeded at a density of 2×10⁶ cells per flask (150 cm²) in cell culture medium and were grown for 7 days. The medium was replaced every two to three days. To ensure a constant expression level of transport protein, L-MDR1 cells were grown in the presence of 640 nM vincristine.

For transport studies, cells were seeded in cell culture medium (as described above but without vincristine) onto 96-well culture plates with microporous polycarbonate inserts (0.4 μm pore size) (Corning Costar plates; Corning LifeSciences) at a density of 2×10⁵ cells/well. The cell culture medium was changed 2 days after seeding and the transport study was performed 3 days post seeding.

Before the start of transport studies, the culture medium was removed, and the cells were washed with transport buffer (HBSS buffer supplemented with 10 mM HEPES) to remove residual amounts of medium. The different compounds tested were dissolved in DMSO and diluted with transport buffer to the final test concentration of 2 μM (final DMSO concentration was 1%). Cells were pre-incubated (37° C., 20 min) on both apical and basolateral surfaces with transport buffer. The experiment was initiated by placing the transport buffer containing the test compounds on the apical (100 μL) or basolateral sides (300 μL) (i.e. the donor compartment) and transport buffer without test compound on the opposite side (i.e. the receiver compartment). After 2 h of incubation at 37° C. on a shaker, samples were taken from both compartments. To demonstrate the tightness of the cell monolayer, the fluorescent dye lucifer yellow was applied on the apical side to measure the transfer to the basolateral compartment. Following the incubation for 2 h, a sample was taken from the basolateral and apical compartment and measured on a fluorescence reader. Before LC-MS/MS analysis, samples were mixed with a mixture of ammonium acetate buffer (pH 6.8) and acetonitrile (1:1, v/v) at equal amounts.

Permeability (Pₐₚₚ) was calculated in the apical to basolateral (A→B) and basolateral to apical (B→A) directions. The apparent permeability was calculated using following equation:

Pₐₚₚ=(Vᵣ/P₀)(1/S)(P₂/t)

Where Vᵣ is the volume of medium in the receiver chamber, P₀ is the measured peak area of the test drug in the donor chamber at t=0, S the surface area of the monolayer, Pa is the measured peak area of the test drug in the acceptor chamber after 2 h of incubation, and t is the incubation time. The efflux ratio was calculated by dividing the Pₐₚₚ B-A by the Pₐₚₚ A-B. An assay control reference P-gp substrate was analyzed in parallel with and without a known P-gp inhibitor.

### Caco-2 Permeability Assay

Caco-2 cells [purchased from the German Collection of Microorganisms and Cell Cultures (DSMZ), Braunschweig, Germany] were seeded at a density of 4.5×10⁴ cells/well on 24-well insert plates, 0.4 μm pore size (Costar) and grown for 13-15 days in DMEM supplemented with 10% FCS, 1% GlutaMAX (100×, Gibco), 100 U/mL penicillin, 100 μg/mL streptomycin (Gibco), and 1% nonessential amino acids (100×, Thermo Fischer Scientific).

Cells were maintained at 37° C. in a humidified 5% CO₂ atmosphere. Medium was changed every 2-3 days. Before the assay was run, the culture medium was replaced by FCS-free transport buffer. For the assessment of monolayer integrity, the transepithelial electrical resistance (TEER) was measured. Test compounds were pre-dissolved in DMSO and added either to the apical or basolateral compartment at a final concentration of 2 μM. The organic solvent in the incubations was limited to ≤1% dimethylsulfoxide (DMSO). Before and after incubation for 2 h at 37° C., samples were taken from both compartments and analyzed by LC-MS/MS after precipitation with MeOH. The apparent permeability coefficient (Pₐₚₚ) was calculated both for the apical to basolateral (A→B) and the basolateral to apical (B→A) direction using the following equation: Pₐₚₚ=(Vᵣ/P₀) (1/S) (P₂/t), where Vᵣ is the volume of medium in the receiver chamber, P₀ is the measured peak area of the test compound in the donor chamber at t=0, S is the surface area of the monolayer, P₂ is the measured peak area of the test compound in the acceptor chamber after incubation for 2 h, and t is the incubation time. The efflux ratio (ER) basolateral (B) to apical (A) was calculated by dividing Pₐₚₚ B-A by Pₐₚₚ A-B. In addition, the compound recovery was calculated. As assay control, reference compounds were analyzed in parallel.

### Method Description—Plasma/Whole Blood Stability (Method 1)

The stability of test compounds in plasma or whole blood is assessed using 0.6 ml EDTA plasma or fresh EDTA blood which is spiked with a distinct concentration (0.3 μM) of drug and mixed well. After 0, 30, 60, and 120 min incubation at 37° C. in a shaker, an aliquot of plasma/whole blood is taken and precipitated with acetonitrile containing internal standard (IS). Samples are stored at −20° C. until analysis and centrifuged for 15 min at 3000×g. The supernatant is analyzed via LC-MS/MS and the stability of compound is assessed (by peak area ratio of compound and IS) as percentage remaining compared to 0 min. Between 80% and 100% the compound is considered stable, between 50% and 79% stability is medium and between 0% and 49% poor.

### Method Description-Plasma/Whole Blood Stability (Method 2)

The plasma and blood stability are investigated in mouse, rat, dog, rabbit, minipig, monkey, and human using 1 mL K3-EDTA plasma or blood with a nominal test concentration of 1000 μg/L at 37° C. for 5 h. At time point 0 h, 0.083 h, 0.25 h, 0.5 h, 1 h, 2 h, 3 h, and 5 h an aliquot of 50 μL is precipitated with 150 μL acetonitrile, which is supplemented with two internal standards. After vortexing and centrifugation for 10 min. at 1300 g an aliquot of 50 μL supernatant is diluted 1/10 with 10 mM ammonium acetate buffer at pH 3 and measured by LC-MS/MS. A concentration-time profile is described in Excel and the respective half-life calculation using t_{1/2}=In2/k is done.

### Method Description—Plasma/Whole Blood Stability (Method 3)

60 μM working solutions of test compounds are prepared in DMSO, 1 mM working solution of control compound propantheline is prepared in acetonitrile. 4 μL of the working solutions is spiked to 796 μL of pre-incubated whole blood to reach a final concentration of 0.3 μM or 5 μM. The final concentration of solvent is 0.5%, 50 μL aliquots of the spiked whole blood are added into new tubes for different time points including 15, 30, 60 and 120 minutes and incubated at 37° C. water bath with shaking at 60 rpm. The assay is performed in duplicate. The reaction is stopped by adding 300 μL of room temperature quench solution (acetonitrile with 0.1% formic acid containing internal standards (IS, 100 nM Alprazolam, 500 nM Labetalol and 2 μM Ketoprofen)) to the spiked whole blood samples at the appointed time points. Time zero samples are prepared by adding 50 μL of the spiked whole blood to new tubes containing 300 μL of room temperature quench solution. Vortex for 5 minutes. Samples in plate are centrifuged at 3,220 g for 30 minutes at 4° C. to precipitate protein. And then 100 μL of the supernatant is transferred to a new 96-well plate with 100 μL water for LC-MS/MS analysis. Calculations are carried out using Microsoft Excel. The in vitro t_{1/2} was determined from the slope value where k is the rate constant (k=-slope value).

### REFERENCES

Arcila et al., 2012: Arcila et al., Clin Cancer Res. 2012 Sep. 15; 18 (18): 4910-8.
Chen et al., 2016: Chen et al., Onco Targets Ther. 2016 Jul. 8; 9:4181-6
Chiu et al., 2015: Chiu et al., J Thorac Oncol. 2015; 10:793-799
Friedlaender et al., 2022: Friedlaender et al., Nat Rev Clin Oncol. 2022 January; 19 (1): 51-69
Gridelli et al., 2015: Gridelli et al., Nat Rev Dis Primers. 2015 May 21; 1:15009.
Mok et al., 2009: Mok et al., N Engl J Med. 2009 Sep. 3; 361 (10): 947-57
Mok et al., 2017: Mok et al., N Engl J Med. 2017 Feb. 16; 376 (7): 629-640
Paez et al., 2004: Paez et al., Science. 2004 Jun. 4; 304 (5676): 1497-500
Pao et al., 2005: Pao et al., PLOS Med. 2005 March; 2 (3): e73
Pao et al., 2010: Pao and Chmielecki, Nat Rev Cancer. 2010 November; 10 (11): 760-74
Rangachari et al., 2015: Rangachari et al., Lung Cancer. 2015 April; 88 (1): 108-11
Reungwetwattana et al., 2018: Reungwetwattana et al., J Clin Oncol. 2018 Nov. 20; 36 (33): 3290-3297
Sequist et al., 2013: Sequist et al., J Clin Oncol. 2013 Sep. 20; 31 (27): 3327-34
Soria et al., 2018: Soria et al., N Engl J Med. 2018 Jan. 11; 378 (2): 113-125.
Yang et al., 2015: Yang et al., Lancet Oncol. 2015 July; 16 (7): 830-8
Yasuda, 2013: Yasuda, Sci Transl Med. 2013 Dec. 18; 5 (216): 216ra177.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A compound of formula (I) 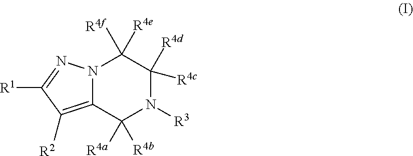
in which:
R¹ represents a group selected from the group: 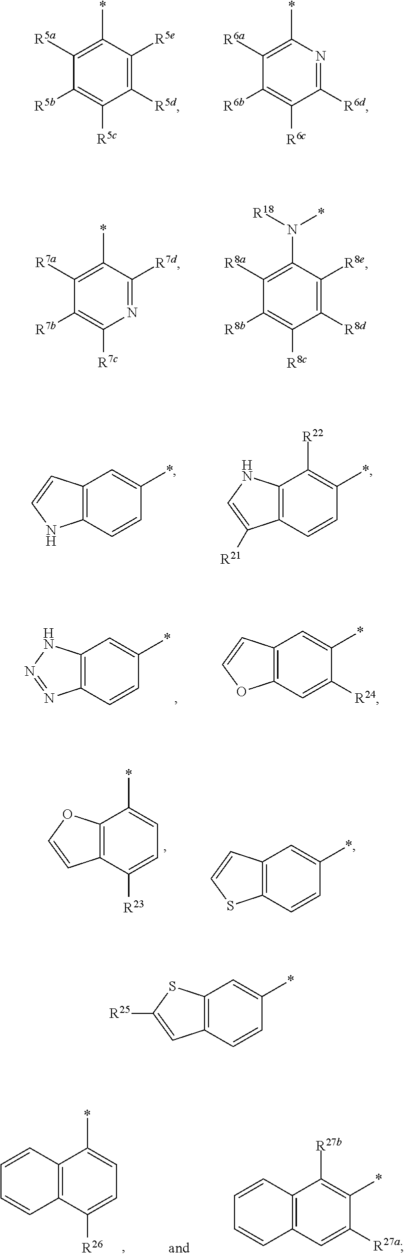
R² represents a group selected from the group: 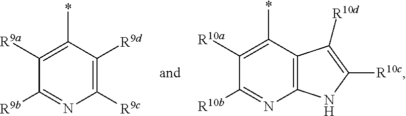
R³ represents —(CO)—R¹¹, —(CO)—C≡C—R¹², —(SO₂)—CH═CH₂, or oxirane-2-carbonyl;
R^{4a} represents a hydrogen atom, fluoro, or methyl;
R^{4b} represents a hydrogen atom, fluoro, or methyl;
R^{4c} represents a hydrogen atom, fluoro, or methyl;
R^{4d} represents a hydrogen atom, fluoro, or methyl;
R^{4e} represents a hydrogen atom, fluoro, or methyl;
R^{4f} represents a hydrogen atom, fluoro, methyl, C₁-C₂-hydroxyalkyl, —(CO)—NR¹⁹R²⁰,
or
CH₂—(CO)—NR¹⁹R²⁰,
R^{5a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, hydroxy, cyano, fluoro, chloro, or bromo;
R^{5b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, trifluoromethylsulfanyl, cyano, fluoro, chloro, or bromo;
R^{5d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{5e} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{6c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{6d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{7c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{7d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{8a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{8d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R⁸⁸ represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{9a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl,
C₃-C₆-cycloalkyl, cyano, fluoro, chloro, or bromo;
R^{9b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{9c} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, C₁-C₃-alkyl-amino, amino-C₁-C₃-alkyl, C₁-C₃-alkylamino-C₁-C₃-alkyl, (C₁-C₃-alkyl)₂amino-C₁-C₃-alkyl, cyano, anilino, pyridin-2-yl-amino, or C₃-C₆-cycloalkylformamido;
R^{9d} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{10a} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, cyano, fluoro, chloro, or bromo;
R^{10b} represents a hydrogen atom, amino, C₁-C₃-alkyl, C₂-C₃-alkenyl, C₂-C₃-alkinyl, C₁-C₃-haloalkyl, C₁-C₃-hydroxyalkyl, C₁-C₃-alkoxy, C₁-C₃-haloalkoxy, C₁-C₃-alkoxy-C₁-C₃-alkyl, amino-C₁-C₃-alkyl, C₁-C₃-alkylamino-C₁-C₃-alkyl, (C₁-C₃-alkyl)₂amino-C₁-C₃-alkyl, or cyano;
R^{10c} represents a hydrogen atom or methyl;
R^{10d} represents a hydrogen atom, fluoro, or methyl;
R¹¹ represents —CH₂—C═CH, —C(R¹⁴)—R¹⁵, or 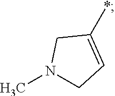
R¹² represents a hydrogen atom, methyl, —CH₂—NR¹⁹R²⁰, or a group selected from the group: 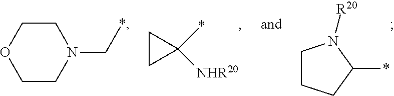
R¹⁴ represents ═CHR¹⁶;
R¹⁵ represents a hydrogen atom, C₁-C₃-alkyl, fluoro, dimethylamino-methyl, or morpholino-methyl;
R¹⁶ represents a hydrogen atom, methyl, chloro, C₁-C₃-haloalkyl, C₁-C₃-hydroxyalkyl, C₁-C₃-alkoxy-C₁-C₃-alkyl, —CH₂—NR^{17a}—CR^{17b}R^{17c}CR^{17d}, or a group selected from the group: 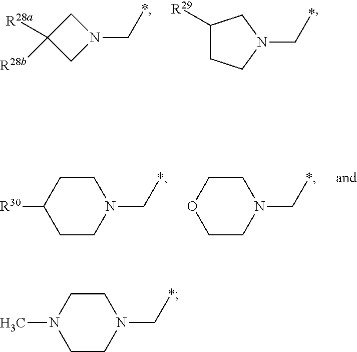
R^{17a} represents a hydrogen atom, methyl, or ethyl;
R^{17b} represents a hydrogen atom or methyl;
R^{17c} represents a hydrogen atom or methyl;
R^{17d} represents a hydrogen atom, methyl, hydroxymethyl, or methoxymethyl;
R¹⁸ represents a hydrogen atom or methyl;
R¹⁹ represents a hydrogen atom or methyl;
R²⁰ represents a hydrogen atom, C₁-C₄-alkyl, or tert-butoxycarbonyl;
R²¹ represents a hydrogen atom or methyl;
R²² represents a hydrogen atom or fluoro;
R²³ represents a hydrogen atom or fluoro;
R²⁴ represents a hydrogen atom or fluoro;
R²⁵ represents a hydrogen atom, fluoro, or chloro;
R²⁶ represents a hydrogen atom or fluoro;
R^{27a} represents a hydrogen atom or fluoro;
R^{27b} represents a hydrogen atom or fluoro;
R^{28a} represents a hydrogen atom or fluoro;
R^{28b} represents a hydrogen atom or fluoro;
R²⁹ represents a hydrogen atom or methoxy;
R³⁰ represents a hydrogen atom or methoxy;
wherein * indicates the point of attachment of said group with the rest of the molecule;
or an N-oxide, a salt, a tautomer, a rotamer, or a stereoisomer of said compound, or a salt of said N-oxide, tautomer, rotamer, or stereoisomer.

2. The compound of formula (I) according to claim 1, wherein:
R¹ represents a group selected from the group: 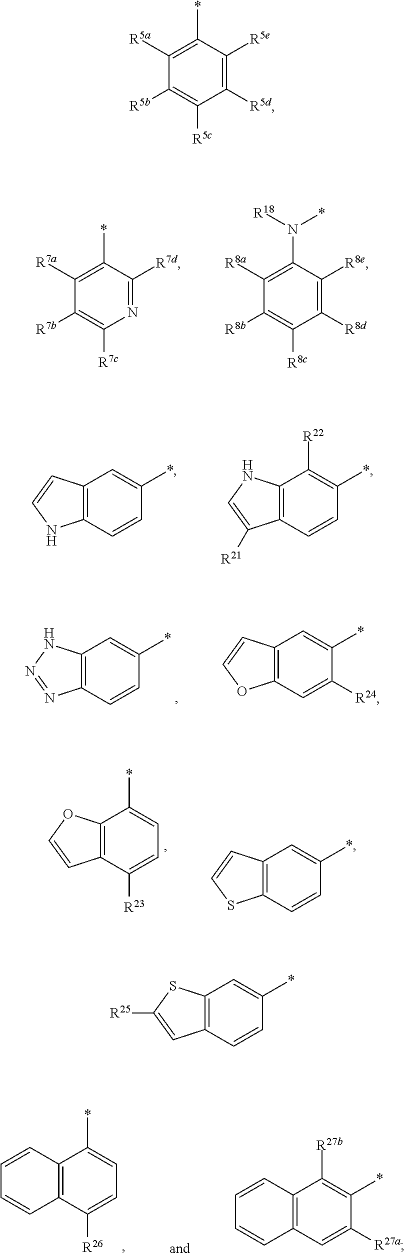
R² represents a group selected from the group: 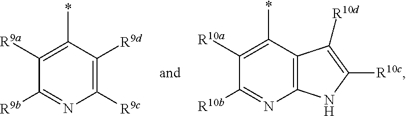
R³ represents —(CO)—R¹¹, —(CO)—C═C—R¹², —(SO₂)—CH═CH₂, or oxirane-2-carbonyl;
R^{4a} represents a hydrogen atom, or methyl;
R^{4b} represents a hydrogen atom;
R^{4c} represents a hydrogen atom, or methyl;
R^{4d} represents a hydrogen atom;
R^{4e} represents a hydrogen atom, or methyl;
R^{4f} represents a hydrogen atom, methyl, C₁-C₂-hydroxyalkyl, —(CO)—NR¹⁹R²⁰, or —CH₂—(CO)—NR¹⁹R²⁰,
R^{5a} represents a hydrogen atom, methyl, hydroxymethyl, methoxy, hydroxy, fluoro, or chloro;
R^{5b} represents a hydrogen atom, C₁-C₂-alkyl, trifluoromethyl, hydroxymethyl, methoxy, cyano, fluoro, or chloro;
R^{5c} represents a hydrogen atom, C₁-C₂-alkyl, ethinyl, C₁-haloalkyl, C₁-C₂-alkoxy, C₁-haloalkoxy, trifluoromethylsulfanyl, cyano, fluoro, or chloro;
R^{5d} represents a hydrogen atom, or
chloro;
R^{5e} represents a hydrogen atom;
R^{7a} represents a hydrogen atom;
R^{7b} represents a hydrogen atom;
R^{7c} represents trifluoromethyl;
R^{7d} represents a hydrogen atom;
R^{8a} represents a hydrogen atom, or fluoro;
R^{8b} represents a hydrogen atom, or fluoro;
R^{8c} represents a hydrogen atom, methoxy, trifluoromethyl, fluoro, or chloro;
R^{8d} represents a hydrogen atom;
R^{8e} represents a hydrogen
atom;
R^{9a} represents a hydrogen
atom;
R^{9b} represents a hydrogen atom;
R^{9c} represents a hydrogen atom, amino, methoxy, methyl-amino, anilino, or cyclopropylformamido;
R^{9d} represents a hydrogen atom, methyl, methoxy, or
cyclopropyl;
R^{10a} represents a hydrogen atom, or fluoro;
R^{10b} represents a hydrogen atom;
R^{10c} represents a hydrogen atom or methyl;
R^{10d} represents a hydrogen atom, fluoro, or methyl;
R¹¹ represents —C(R¹⁴)—R¹⁵, or 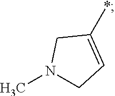
R¹² represents a hydrogen atom, methyl, —CH₂—NR¹⁹R²⁰, or a group selected from the group: 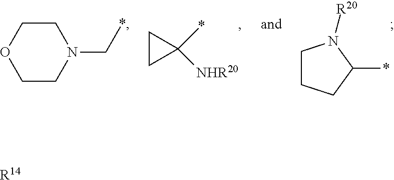
represents ═CHR¹⁶;
R¹⁵ represents a hydrogen atom, methyl, fluoro, dimethylamino-methyl, or morpholino-methyl;
R¹⁶ represents a hydrogen atom, methyl, chloro, C₁-haloalkyl, hydroxymethyl, methoxymethyl, —CH₂—NR^{17a}—CR^{17b}R^{17c}R^{17d}, or a group selected from the 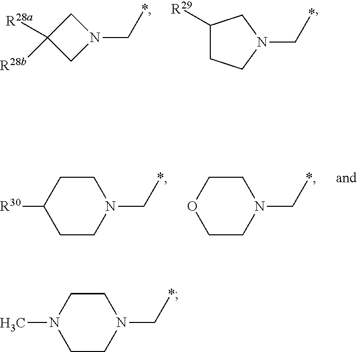
R^{17a} represents a hydrogen atom, methyl, or
ethyl;
R^{17b} represents a hydrogen atom or methyl;
R^{17c} represents a hydrogen atom or methyl;
R^{17d} represents a hydrogen atom, methyl, hydroxymethyl, or methoxymethyl;
R¹⁸ represents a hydrogen atom;
R¹⁹ represents a hydrogen atom or methyl;
R²⁰ represents a hydrogen atom, methyl, or tert-butoxycarbonyl;
R²¹ represents a hydrogen atom or methyl;
R²² represents a hydrogen atom or fluoro;
R²³ represents a hydrogen atom or fluoro;
R²⁴ represents a hydrogen atom or fluoro;
R²⁵ represents a hydrogen atom, fluoro, or chloro;
R²⁶ represents a hydrogen atom or fluoro;
R^{27a} represents a hydrogen atom or fluoro;
R^{27b} represents a hydrogen atom or fluoro;
R^{28a} represents a hydrogen atom or fluoro;
R^{28b} represents a hydrogen atom or fluoro;
R²⁹ represents a hydrogen atom or
methoxy;
R³⁰ represents a hydrogen atom or
methoxy;
wherein * indicates the point of attachment of said group with the rest of the molecule;
or an N-oxide, a salt, a tautomer, a rotamer, or a stereoisomer of said compound, or a salt of said N-oxide, tautomer, rotamer, or stereoisomer.

3. The compound of formula (1) according to claim 1, which is selected from the group consisting of:
1-[2-(4-fluorophenyl)-3-(pyridin-4-yl)-6,7-dihydropyrazolo[1,5-a]pyrazin-5(4H)-yl]prop-2-en-1-one,
1-[2-(4-chloro-3-methylphenyl)-3-(pyridin-4-yl)-6,7-dihydropyrazolo[1,5-a]pyrazin-5(4H)-yl]prop-2-en-1-one,
1-[2-(4-chlorophenyl)-3-(pyridin-4-yl)-6,7-dihydropyrazolo[1,5-a]pyrazin-5(4H)-yl]prop-2-en-1-one,
1-[2-(3-fluorophenyl)-3-(pyridin-4-yl)-6,7-dihydropyrazolo[1,5-a]pyrazin-5(4H)-yl]prop-2-en-1-one,
1-[2-(3-methoxyphenyl)-3-(pyridin-4-yl)-6,7-dihydropyrazolo[1,5-a]pyrazin-5(4H)-yl]prop-2-en-1-one,
N-{4-[2-(4-fluorophenyl)-5-{(2E)-4-[methyl(propan-2-yl)amino]but-2-enoyl}-4,5,6,7-tetrahydropyrazolo[1,5-a]pyrazin-3-yl]pyridin-2-yl}cyclopropanecarboxamide,
N-{4-[2-(4-fluorophenyl)-5-(prop-2-enoyl)-4,5,6,7-tetrahydropyrazolo[1,5-a]pyrazin-3-yl]pyridin-2-yl}cyclopropanecarboxamide,
N-(4-{5-[(2E)-4-(dimethylamino)but-2-enoyl]-2-(4-fluorophenyl)-4,5,6,7-tetrahydropyrazolo[1,5-a]pyrazin-3-yl}pyridin-2-yl)cyclopropanecarboxamide,
N-{4-[2-(4-fluorophenyl)-5-(oxirane-2-carbonyl)-4,5,6,7-tetrahydropyrazolo[1,5-a]pyrazin-3-yl]pyridin-2-yl}cyclopropanecarboxamide,
1-[3-(2-aminopyridin-4-yl)-2-(4-fluorophenyl)-6,7-dihydropyrazolo[1,5-a]pyrazin-5(4H)-yl]prop-2-en-1-one,
1-[2-(4-fluorophenyl)-3-(2-methoxypyridin-4-yl)-6,7-dihydropyrazolo[1,5-a]pyrazin-5(4H)-yl]prop-2-en-1-one,
1-[2-(4-fluorophenyl)-3-(3-methoxypyridin-4-yl)-6,7-dihydropyrazolo[1,5-a]pyrazin-5(4H)-yl]prop-2-en-1-one,
1-[2-(4-fluorophenyl)-3-(pyridin-4-yl)-6,7-dihydropyrazolo[1,5-a]pyrazin-5(4H)-yl]prop-2-yn-1-one,
5-(ethenesulfonyl)-2-(4-fluorophenyl)-3-(pyridin-4-yl)-4,5,6,7-tetrahydropyrazolo[1,5-a]pyrazine,
(2E)-4-(dimethylamino)-1-[2-(4-fluorophenyl)-3-(pyridin-4-yl)-6,7-dihydropyrazolo[1,5-a]pyrazin-5(4H)-yl]but-2-en-1-one,
1-[2-(4-fluorophenyl)-3-(pyridin-4-yl)-6,7-dihydropyrazolo[1,5-a]pyrazin-5(4H)-yl]but-2-yn-1-one,
2-fluoro-1-[2-(4-fluorophenyl)-3-(pyridin-4-yl)-6,7-dihydropyrazolo[1,5-a]pyrazin-5(4H)-yl]prop-2-en-1-one,
(2E)-1-[2-(4-fluorophenyl)-3-(pyridin-4-yl)-6,7-dihydropyrazolo[1,5-a]pyrazin-5(4H)-yl]-4-[methyl(propan-2-yl)amino]but-2-en-1-one,
1-[2-(4-fluoroanilino)-3-(pyridin-4-yl)-6,7-dihydropyrazolo[1,5-a]pyrazin-5(4H)-yl]prop-2-en-1-one,
1-[3-(2-anilinopyridin-4-yl)-2-(4-fluorophenyl)-6,7-dihydropyrazolo[1,5-a]pyrazin-5(4H)-yl]prop-2-en-1-one,
(2E)-1-[3-(2-anilinopyridin-4-yl)-2-(4-fluorophenyl)-6,7-dihydropyrazolo[1,5-a]pyrazin-5(4H)-yl]-4-(dimethylamino)but-2-en-1-one,
1-[2-(4-fluorophenyl)-3-(1H-pyrrolo[2,3-b]pyridin-4-yl)-6,7-dihydropyrazolo[1,5-a]pyrazin-5(4H)-yl]prop-2-en-1-one,
(2E)-1-[2-(4-fluorophenyl)-3-(pyridin-4-yl)-6,7-dihydropyrazolo[1,5-a]pyrazin-5(4H)-yl]but-2-en-1-one,
1-[2-(4-fluorophenyl)-3-(pyridin-4-yl)-6,7-dihydropyrazolo[1,5-a]pyrazin-5(4H)-yl]-2-methylprop-2-en-1-one,
(2E)-1-[2-(4-chloro-3-fluorophenyl)-3-(pyridin-4-yl)-6,7-dihydropyrazolo[1,5-a]pyrazin-5(4H)-yl]-4-(dimethylamino)but-2-en-1-one
(2E)-1-[2-(4-chloro-2-fluorophenyl)-3-(pyridin-4-yl)-6,7-dihydropyrazolo[1,5-a]pyrazin-5(4H)-yl]-4-(dimethylamino)but-2-en-1-one
(2E)-1-[2-(3-chloro-4-methoxyphenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(2-methoxyphenyl)-3-(pyridin-4-yl)-6,7-dihydropyrazolo[1,5-a]pyrazin-5(4H)-yl]but-2-en-1-one
(2E)-1-[2-(3,4-dichlorophenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(4-methoxyphenyl)-3-(pyridin-4-yl)-6,7-dihydropyrazolo[1,5-a]pyrazin-5(4H)-yl]but-2-en-1-one
(2E)-4-(dimethylamino)-1-[2-(3-fluoro-4-methoxyphenyl)-3-(pyridin-4-yl)-6,7-dihydropyrazolo[1,5-a]pyrazin-5(4H)-yl]but-2-en-1-one formic acid-(2E)-1-[2-(4-chloro-3-ethylphenyl)-3-(pyridin-4-yl)-6,7-dihydropyrazolo[1,5-a]pyrazin-5(4H)-yl]-4-(dimethylamino)but-2-en-1-one
4-{5-[(2E)-4-(dimethylamino)but-2-enoyl]-3-(pyridin-4-yl)-4,5,6,7-tetrahydropyrazolo[1,5-a]pyrazin-2-yl}benzonitrile
(2E)-4-(dimethylamino)-1-[3-(pyridin-4-yl)-2-[4-(trifluoromethyl)phenyl]-6,7-dihydropyrazolo[1,5-a]pyrazin-5(4H)-yl]but-2-en-1-one
(2E)-1-{2-[4-chloro-2-(hydroxymethyl)phenyl]-3-(pyridin-4-yl)-6,7-dihydropyrazolo[1,5-a]pyrazin-5(4H)-yl}-4-(dimethylamino)but-2-en-1-one
1-[2-(4-chloro-3-fluorophenyl)-3-(pyridin-4-yl)-6,7-dihydropyrazolo[1,5-a]pyrazin-5(4H)-yl]prop-2-en-1-one
1-[2-(4-chloro-2-fluorophenyl)-3-(pyridin-4-yl)-6,7-dihydropyrazolo[1,5-a]pyrazin-5(4H)-yl]prop-2-en-1-one
1-[2-(3-chloro-4-methoxyphenyl)-3-(pyridin-4-yl)-6,7-dihydropyrazolo[1,5-a]pyrazin-5(4H)-yl]prop-2-en-1-one
1-[2-(2-methoxyphenyl)-3-(pyridin-4-yl)-6,7-dihydropyrazolo[1,5-a]pyrazin-5(4H)-yl]prop-2-en-1-one
1-[2-(3,4-dichlorophenyl)-3-(pyridin-4-yl)-6,7-dihydropyrazolo[1,5-a]pyrazin-5(4H)-yl]prop-2-en-1-one
1-[2-(4-methoxyphenyl)-3-(pyridin-4-yl)-6,7-dihydropyrazolo[1,5-a]pyrazin-5(4H)-yl]prop-2-en-1-one
1-[2-(3-fluoro-4-methoxyphenyl)-3-(pyridin-4-yl)-6,7-dihydropyrazolo[1,5-a]pyrazin-5(4H)-yl]prop-2-en-1-one
4-[5-acryloyl-3-(pyridin-4-yl)-4,5,6,7-tetrahydropyrazolo[1,5-a]pyrazin-2-yl]benzonitrile
1-[3-(pyridin-4-yl)-2-[4-(trifluoromethyl)phenyl]-6,7-dihydropyrazolo[1,5-a]pyrazin-5(4H)-yl]prop-2-en-1-one
3-[3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
1-[2-(3-fluoro-4-methoxyphenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(4-methoxyphenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-[2-(4-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(4-chloro-2-fluorophenyl)-3-(3-methylpyridin-4-yl)-6,7-dihydropyrazolo[1,5-a]pyrazin-5(4H)-yl]prop-2-en-1-one
1-[2-(4-chloro-2-fluorophenyl)-3-(5-fluoro-1H-pyrrolo[2,3-b]pyridin-4-yl)-6,7-dihydropyrazolo[1,5-a]pyrazin-5(4H)-yl]prop-2-en-1-one
1-[2-(4-chloro-2-fluorophenyl)-3-(3-fluoro-1H-pyrrolo[2,3-b]pyridin-4-yl)-6,7-dihydropyrazolo[1,5-a]pyrazin-5(4H)-yl]prop-2-en-1-one
1-[2-(4-chloro-2-fluorophenyl)-3-[2-(methylamino)pyridin-4-yl]-6,7-dihydropyrazolo[1,5-a]pyrazin-5(4H)-yl]prop-2-en-1-one
1-[3-(2-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-[3-(3-cyclopropylpyridin-4-yl)-2-[4-(trifluoromethyl)phenyl]-6,7-dihydropyrazolo[1,5-a]pyrazin-5(4H)-yl]prop-2-en-1-one
1-[3-(2,3-dimethyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
(2E)-1-[2-(4-chloro-2-fluorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one
(2E)-4-(dimethylamino)-1-[3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
(2E)-4-(dimethylamino)-1-[2-(3-fluoro-4-methoxyphenyl)-3-(3-methyl-1H-pyrrolo[2,3-b]pyridin-4-yl)-6,7-dihydropyrazolo[1,5-a]pyrazin-5(4H)-yl]but-2-en-1-one
(2E)-4-(dimethylamino)-1-[2-(4-methoxyphenyl)-3-(3-methyl-1H-pyrrolo[2,3-b]pyridin-4-yl)-6,7-dihydropyrazolo[1,5-a]pyrazin-5(4H)-yl]but-2-en-1-one
3-{5-[(2E)-4-(dimethylamino)but-2-enoyl]-3-(3-methyl-1H-pyrrolo[2,3-b]pyridin-4-yl)-4,5,6,7-tetrahydropyrazolo[1,5-a]pyrazin-2-yl}benzonitrile
2-[(dimethylamino)methyl]-1-[2-(4-fluorophenyl)-3-(pyridin-4-yl)-6,7-dihydropyrazolo[1,5-a]pyrazin-5(4H)-yl]prop-2-en-1-one
(2E)-1-[2-(4-fluorophenyl)-3-(pyridin-4-yl)-6,7-dihydropyrazolo[1,5-a]pyrazin-5(4H)-yl]-4-(morpholin-4-yl) but-2-en-1-one
1-[2-(4-fluorophenyl)-3-(pyridin-4-yl)-6,7-dihydropyrazolo[1,5-a]pyrazin-5(4H)-yl]-2-[(morpholin-4-yl)methyl]prop-2-en-1-one
4-(dimethylamino)-1-[2-(4-fluorophenyl)-3-(pyridin-4-yl)-6,7-dihydropyrazolo[1,5-a]pyrazin-5(4H)-yl]but-2-yn-1-one
(2E)-1-[2-(4-fluorophenyl)-3-(pyridin-4-yl)-6,7-dihydropyrazolo[1,5-a]pyrazin-5(4H)-yl]-4-methoxybut-2-en-1-one
(2E)-4,4,4-trifluoro-1-[2-(4-fluorophenyl)-3-(pyridin-4-yl)-6,7-dihydropyrazolo[1,5-a]pyrazin-5(4H)-yl]but-2-en-1-one
1-[2-(4-fluorophenyl)-3-(pyridin-4-yl)-6,7-dihydropyrazolo[1,5-a]pyrazin-5(4H)-yl]-4-(morpholin-4-yl) but-2-yn-1-one
(2E)-4-(diethylamino)-1-[2-(4-fluorophenyl)-3-(pyridin-4-yl)-6,7-dihydropyrazolo[1,5-a]pyrazin-5(4H)-yl]but-2-en-1-one
(2E)-1-[2-(4-fluorophenyl)-3-(pyridin-4-yl)-6,7-dihydropyrazolo[1,5-a]pyrazin-5(4H)-yl]-4-hydroxybut-2-en-1-one
(2E)-4-[ethyl(methyl)amino]-1-[2-(4-fluorophenyl)-3-(pyridin-4-yl)-6,7-dihydropyrazolo[1,5-a]pyrazin-5(4H)-yl]but-2-en-1-one
1-[2-(4-chloro-2-fluorophenyl)-3-(pyridin-4-yl)-6,7-dihydropyrazolo[1,5-a]pyrazin-5(4H)-yl]-4-(dimethylamino)but-2-yn-1-one
(2E)-1-[2-(4-chloro-2-fluorophenyl)-3-(pyridin-4-yl)-6,7-dihydropyrazolo[1,5-a]pyrazin-5(4H)-yl]-4-fluorobut-2-en-1-one
(2E)-1-[2-(4-chloro-2-fluorophenyl)-3-(pyridin-4-yl)-6,7-dihydropyrazolo[1,5-a]pyrazin-5(4H)-yl]-4,4-difluorobut-2-en-1-one
(2E)-3-chloro-1-[2-(4-chloro-2-fluorophenyl)-3-(pyridin-4-yl)-6,7-dihydropyrazolo[1,5-a]pyrazin-5(4H)-yl]prop-2-en-1-one
[2-(4-chloro-2-fluorophenyl)-3-(pyridin-4-yl)-6,7-dihydropyrazolo[1,5-a]pyrazin-5(4H)-yl](1-methyl-2,5-dihydro-1H-pyrrol-3-yl) methanone
(2E)-4-(azetidin-1-yl)-1-[2-(4-chloro-2-fluorophenyl)-3-(pyridin-4-yl)-6,7-dihydropyrazolo[1,5-a]pyrazin-5(4H)-yl]but-2-en-1-one
(2E)-4-fluoro-1-[2-(4-fluorophenyl)-3-(pyridin-4-yl)-6,7-dihydropyrazolo[1,5-a]pyrazin-5(4H)-yl]but-2-en-1-one
(2E)-1-[2-(4-fluorophenyl)-3-(pyridin-4-yl)-6,7-dihydropyrazolo[1,5-a]pyrazin-5(4H)-yl]-4-(pyrrolidin-1-yl) but-2-en-1-one
(2E)-1-[2-(4-fluorophenyl)-3-(pyridin-4-yl)-6,7-dihydropyrazolo[1,5-a]pyrazin-5(4H)-yl]-4-(methylamino)but-2-en-1-one
(2E)-4-(ethylamino)-1-[2-(4-fluorophenyl)-3-(pyridin-4-yl)-6,7-dihydropyrazolo[1,5-a]pyrazin-5(4H)-yl]but-2-en-1-one
(2E)-1-[2-(4-fluorophenyl)-3-(pyridin-4-yl)-6,7-dihydropyrazolo[1,5-a]pyrazin-5(4H)-yl]-4-(4-methylpiperazin-1-yl) but-2-en-1-one
(2E)-1-[2-(4-fluorophenyl)-3-(pyridin-4-yl)-6,7-dihydropyrazolo[1,5-a]pyrazin-5(4H)-yl]-4-[(3S)-3-methoxypyrrolidin-1-yl]but-2-en-1-one
(2E)-4-(3,3-difluoroazetidin-1-yl)-1-[2-(4-fluorophenyl)-3-(pyridin-4-yl)-6,7-dihydropyrazolo[1,5-a]pyrazin-5(4H)-yl]but-2-en-1-one
1-[2-(4-chloro-2-fluorophenyl)-3-(pyridin-4-yl)-6,7-dihydropyrazolo[1,5-a]pyrazin-5(4H)-yl]-4-(morpholin-4-yl) but-2-yn-1-one
tert-butyl(2RS)-2-{3-[2-(4-fluorophenyl)-3-(pyridin-4-yl)-6,7-dihydropyrazolo[1,5-a]pyrazin-5(4H)-yl]-3-oxoprop-1-yn-1-yl}pyrrolidine-1-carboxylate
1-[2-(4-fluorophenyl)-3-(pyridin-4-yl)-6,7-dihydropyrazolo[1,5-a]pyrazin-5(4H)-yl]-3-[(2RS)-pyrrolidin-2-yl]prop-2-yn-1-one
3-(1-aminocyclopropyl)-1-[2-(4-fluorophenyl)-3-(pyridin-4-yl)-6,7-dihydropyrazolo[1,5-a]pyrazin-5(4H)-yl]prop-2-yn-1-one
tert-butyl {4-[2-(4-fluorophenyl)-3-(pyridin-4-yl)-6,7-dihydropyrazolo[1,5-a]pyrazin-5(4H)-yl]-4-oxobut-2-yn-1-yl}carbamate
4-amino-1-[2-(4-fluorophenyl)-3-(pyridin-4-yl)-6,7-dihydropyrazolo[1,5-a]pyrazin-5(4H)-yl]but-2-yn-1-one
1-[2-(4-chloro-2-fluoroanilino)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(3-fluoro-4-methoxyanilino)-3-(3-methyl-1H-pyrrolo[2,3-b]pyridin-4-yl)-6,7-dihydropyrazolo[1,5-a]pyrazin-5(4H)-yl]prop-2-en-1-one
1-[3-(3-methyl-1H-pyrrolo[2,3-b]pyridin-4-yl)-2-[4-(trifluoromethyl) anilino]-6,7-dihydropyrazolo[1,5-a]pyrazin-5(4H)-yl]prop-2-en-1-one
1-[2-(4-chloro-2-fluoroanilino)-3-(pyridin-4-yl)-6,7-dihydropyrazolo[1,5-a]pyrazin-5(4H)-yl]prop-2-en-1-one
1-[2-(3-fluoro-4-methoxyanilino)-3-(pyridin-4-yl)-6,7-dihydropyrazolo[1,5-a]pyrazin-5(4H)-yl]prop-2-en-1-one
1-[3-(pyridin-4-yl)-2-[4-(trifluoromethyl) anilino]-6,7-dihydropyrazolo[1,5-a]pyrazin-5(4H)-yl]prop-2-en-1-one
(2E)-4-(dimethylamino)-1-[2-(4-fluorophenyl)-3-(1H-pyrrolo[2,3-b]pyridin-4-yl)-6,7-dihydropyrazolo[1,5-a]pyrazin-5(4H)-yl]but-2-en-1-one
(2E)-4-(dimethylamino)-1-[2-(2-fluoro-4-methylphenyl)-3-(1H-pyrrolo[2,3-b]pyridin-4-yl)-6,7-dihydropyrazolo[1,5-a]pyrazin-5(4H)-yl]but-2-en-1-one
3-[(6R)-5-[(2E)-4-(dimethylamino)but-2-enoyl]-6-methyl-3-(pyridin-4-yl)-4,5,6,7-tetrahydropyrazolo[1,5-a]pyrazin-2-yl]benzonitrile
3-[(6S)-5-[(2E)-4-(dimethylamino)but-2-enoyl]-6-methyl-3-(pyridin-4-yl)-4,5,6,7-tetrahydropyrazolo[1,5-a]pyrazin-2-yl]benzonitrile
1-[2-(2-fluoro-4-methylphenyl)-3-(1H-pyrrolo[2,3-b]pyridin-4-yl)-6,7-dihydropyrazolo[1,5-a]pyrazin-5(4H)-yl]prop-2-en-1-one
(2E)-1-[2-(4-chloro-2-fluorophenyl)-3-(1H-pyrrolo[2,3-b]pyridin-4-yl)-6,7-dihydropyrazolo[1,5-a]pyrazin-5(4H)-yl]-4-(dimethylamino)but-2-en-1-one
1-[2-(4-chloro-2-fluorophenyl)-3-(1H-pyrrolo[2,3-b]pyridin-4-yl)-6,7-dihydropyrazolo[1,5-a]pyrazin-5(4H)-yl]prop-2-en-1-one
1-[2-(4-fluoro-1-benzofuran-7-yl)-3-(pyridin-4-yl)-6,7-dihydropyrazolo[1,5-a]pyrazin-5(4H)-yl]prop-2-en-1-one
1-[2-(4-fluoronaphthalen-1-yl)-3-(pyridin-4-yl)-6,7-dihydropyrazolo[1,5-a]pyrazin-5(4H)-yl]prop-2-en-1-one
1-[2-(1-benzofuran-7-yl)-3-(pyridin-4-yl)-6,7-dihydropyrazolo[1,5-a]pyrazin-5(4H)-yl]prop-2-en-1-one
1-[2-(3-methyl-1H-indol-6-yl)-3-(pyridin-4-yl)-6,7-dihydropyrazolo[1,5-a]pyrazin-5(4H)-yl]prop-2-en-1-one
1-[2-(3-fluoronaphthalen-2-yl)-3-(pyridin-4-yl)-6,7-dihydropyrazolo[1,5-a]pyrazin-5(4H)-yl]prop-2-en-1-one
1-[2-(6-fluoro-1-benzofuran-5-yl)-3-(pyridin-4-yl)-6,7-dihydropyrazolo[1,5-a]pyrazin-5(4H)-yl]prop-2-en-1-one
1-[2-(1-fluoronaphthalen-2-yl)-3-(pyridin-4-yl)-6,7-dihydropyrazolo[1,5-a]pyrazin-5(4H)-yl]prop-2-en-1-one
1-[2-(2-hydroxy-4-methylphenyl)-3-(pyridin-4-yl)-6,7-dihydropyrazolo[1,5-a]pyrazin-5(4H)-yl]prop-2-en-1-one
1-[2-(2-fluoro-4-methylphenyl)-3-(pyridin-4-yl)-6,7-dihydropyrazolo[1,5-a]pyrazin-5(4H)-yl]prop-2-en-1-one
1-[2-(7-fluoro-1H-indol-6-yl)-3-(pyridin-4-yl)-6,7-dihydropyrazolo[1,5-a]pyrazin-5(4H)-yl]prop-2-en-1-one
1-[2-(4-chloro-2-methylphenyl)-3-(pyridin-4-yl)-6,7-dihydropyrazolo[1,5-a]pyrazin-5(4H)-yl]prop-2-en-1-one
1-[2-(2,4-dichlorophenyl)-3-(pyridin-4-yl)-6,7-dihydropyrazolo[1,5-a]pyrazin-5(4H)-yl]prop-2-en-1-one
1-[2-(4-chloro-2-methoxyphenyl)-3-(pyridin-4-yl)-6,7-dihydropyrazolo[1,5-a]pyrazin-5(4H)-yl]prop-2-en-1-one
2-chloro-5-[5-(prop-2-enoyl)-3-(pyridin-4-yl)-4,5,6,7-tetrahydropyrazolo[1,5-a]pyrazin-2-yl]benzonitrile
2-fluoro-5-[5-(prop-2-enoyl)-3-(pyridin-4-yl)-4,5,6,7-tetrahydropyrazolo[1,5-a]pyrazin-2-yl]benzonitrile
1-[(6R)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one
2-fluoro-3-[5-(prop-2-enoyl)-3-(pyridin-4-yl)-4,5,6,7-tetrahydropyrazolo[1,5-a]pyrazin-2-yl]benzonitrile
1-{2-[3-(hydroxymethyl)phenyl]-3-(pyridin-4-yl)-6,7-dihydropyrazolo[1,5-a]pyrazin-5(4H)-yl}prop-2-en-1-one
1-[(6S)-2-(4-chlorophenyl)-6-methyl-3-(pyridin-4-yl)-6,7-dihydropyrazolo[1,5-a]pyrazin-5(4H)-yl]prop-2-en-1-one
1-[(6R)-2-(4-chlorophenyl)-6-methyl-3-(pyridin-4-yl)-6,7-dihydropyrazolo[1,5-a]pyrazin-5(4H)-yl]prop-2-en-1-one
1-[(6S)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one
3-[(6R)-6-methyl-5-(prop-2-enoyl)-3-(pyridin-4-yl)-4,5,6,7-tetrahydropyrazolo[1,5-a]pyrazin-2-yl]benzonitrile
3-[(6S)-6-methyl-5-(prop-2-enoyl)-3-(pyridin-4-yl)-4,5,6,7-tetrahydropyrazolo[1,5-a]pyrazin-2-yl]benzonitrile
1-{2-[4-(difluoromethoxy)phenyl]-3-(pyridin-4-yl)-6,7-dihydropyrazolo[1,5-a]pyrazin-5(4H)-yl}prop-2-en-1-one
1-[3-(pyridin-4-yl)-2-[4-(trifluoromethoxy)phenyl]-6,7-dihydropyrazolo[1,5-a]pyrazin-5(4H)-yl]prop-2-en-1-one
1-[2-(4-ethoxyphenyl)-3-(pyridin-4-yl)-6,7-dihydropyrazolo[1,5-a]pyrazin-5(4H)-yl]prop-2-en-1-one
1-{2-[4-(difluoromethyl)phenyl]-3-(pyridin-4-yl)-6,7-dihydropyrazolo[1,5-a]pyrazin-5(4H)-yl}prop-2-en-1-one
1-[2-(5-chloro-2-fluorophenyl)-3-(pyridin-4-yl)-6,7-dihydropyrazolo[1,5-a]pyrazin-5(4H)-yl]prop-2-en-1-one
1-[2-(3-chlorophenyl)-3-(pyridin-4-yl)-6,7-dihydropyrazolo[1,5-a]pyrazin-5(4H)-yl]prop-2-en-1-one
1-{2-[3-fluoro-4-(trifluoromethyl)phenyl]-3-(pyridin-4-yl)-6,7-dihydropyrazolo[1,5-a]pyrazin-5(4H)-yl}prop-2-en-1-one
1-{2-[2-fluoro-4-(trifluoromethyl)phenyl]-3-(pyridin-4-yl)-6,7-dihydropyrazolo[1,5-a]pyrazin-5(4H)-yl}prop-2-en-1-one
1-[2-(2-fluorophenyl)-3-(pyridin-4-yl)-6,7-dihydropyrazolo[1,5-a]pyrazin-5(4H)-yl]prop-2-en-1-one
1-[2-(2-chlorophenyl)-3-(pyridin-4-yl)-6,7-dihydropyrazolo[1,5-a]pyrazin-5(4H)-yl]prop-2-en-1-one
1-[2-(4-methylphenyl)-3-(pyridin-4-yl)-6,7-dihydropyrazolo[1,5-a]pyrazin-5(4H)-yl]prop-2-en-1-one
1-[2-(3-methylphenyl)-3-(pyridin-4-yl)-6,7-dihydropyrazolo[1,5-a]pyrazin-5(4H)-yl]prop-2-en-1-one
1-[2-(4-ethylphenyl)-3-(pyridin-4-yl)-6,7-dihydropyrazolo[1,5-a]pyrazin-5(4H)-yl]prop-2-en-1-one
3-[5-(prop-2-enoyl)-3-(pyridin-4-yl)-4,5,6,7-tetrahydropyrazolo[1,5-a]pyrazin-2-yl]benzonitrile
1-[2-(2-fluoro-4-methoxyphenyl)-3-(pyridin-4-yl)-6,7-dihydropyrazolo[1,5-a]pyrazin-5(4H)-yl]prop-2-en-1-one
1-[3-(pyridin-4-yl)-2-{4-[(trifluoromethyl) sulfanyl]phenyl}-6,7-dihydropyrazolo[1,5-a]pyrazin-5(4H)-yl]prop-2-en-1-one
(2E)-1-{2-[4-(difluoromethoxy)phenyl]-3-(pyridin-4-yl)-6,7-dihydropyrazolo[1,5-a]pyrazin-5(4H)-yl}-4-(dimethylamino)but-2-en-1-one
(2E)-4-(dimethylamino)-1-[3-(pyridin-4-yl)-2-[4-(trifluoromethoxy)phenyl]-6,7-dihydropyrazolo[1,5-a]pyrazin-5(4H)-yl]but-2-en-1-one
(2E)-4-(dimethylamino)-1-[2-(4-ethynylphenyl)-3-(pyridin-4-yl)-6,7-dihydropyrazolo[1,5-a]pyrazin-5(4H)-yl]but-2-en-1-one
(2E)-4-(dimethylamino)-1-[2-(4-ethoxyphenyl)-3-(pyridin-4-yl)-6,7-dihydropyrazolo[1,5-a]pyrazin-5(4H)-yl]but-2-en-1-one
(2E)-1-{2-[4-(difluoromethyl)phenyl]-3-(pyridin-4-yl)-6,7-dihydropyrazolo[1,5-a]pyrazin-5(4H)-yl}-4-(dimethylamino)but-2-en-1-one
(2E)-1-[2-(5-chloro-2-fluorophenyl)-3-(pyridin-4-yl)-6,7-dihydropyrazolo[1,5-a]pyrazin-5(4H)-yl]-4-(dimethylamino)but-2-en-1-one
(2E)-1-[2-(3-chlorophenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-{2-[3-fluoro-4-(trifluoromethyl)phenyl]-3-(pyridin-4-yl)-6,7-dihydropyrazolo[1,5-a]pyrazin-5(4H)-yl}but-2-en-1-one
(2E)-4-(dimethylamino)-1-{2-[2-fluoro-4-(trifluoromethyl)phenyl]-3-(pyridin-4-yl)-6,7-dihydropyrazolo[1,5-a]pyrazin-5(4H)-yl}but-2-en-1-one
(2E)-4-(dimethylamino)-1-[2-(2-fluorophenyl)-3-(pyridin-4-yl)-6,7-dihydropyrazolo[1,5-a]pyrazin-5(4H)-yl]but-2-en-1-one
(2E)-1-[2-(2-chlorophenyl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(3-methoxyphenyl)-3-(pyridin-4-yl)-6,7-dihydropyrazolo[1,5-a]pyrazin-5(4H)-yl]but-2-en-1-one
(2E)-4-(dimethylamino)-1-[2-(4-methylphenyl)-3-(pyridin-4-yl)-6,7-dihydropyrazolo[1,5-a]pyrazin-5(4H)-yl]but-2-en-1-one
(2E)-4-(dimethylamino)-1-[2-(3-methylphenyl)-3-(pyridin-4-yl)-6,7-dihydropyrazolo[1,5-a]pyrazin-5(4H)-yl]but-2-en-1-one
(2E)-4-(dimethylamino)-1-[2-(4-ethylphenyl)-3-(pyridin-4-yl)-6,7-dihydropyrazolo[1,5-a]pyrazin-5(4H)-yl]but-2-en-1-one
3-{5-[(2E)-4-(dimethylamino)but-2-enoyl]-3-(pyridin-4-yl)-4,5,6,7-tetrahydropyrazolo[1,5-a]pyrazin-2-yl}benzonitrile
(2E)-4-(dimethylamino)-1-[2-(2-fluoro-4-methoxyphenyl)-3-(pyridin-4-yl)-6,7-dihydropyrazolo[1,5-a]pyrazin-5(4H)-yl]but-2-en-1-one
(2E)-4-(dimethylamino)-1-[3-(pyridin-4-yl)-2-{4-[(trifluoromethyl) sulfanyl]phenyl}-6,7-dihydropyrazolo[1,5-a]pyrazin-5(4H)-yl]but-2-en-1-one
(2E)-4-(azetidin-1-yl)-1-[2-(4-chlorophenyl)-3-(pyridin-4-yl)-6,7-dihydropyrazolo[1,5-a]pyrazin-5(4H)-yl]but-2-en-1-one
(2E)-1-[2-(4-chlorophenyl)-3-(pyridin-4-yl)-6,7-dihydropyrazolo[1,5-a]pyrazin-5(4H)-yl]-4-(piperidin-1-yl) but-2-en-1-one
(2E)-1-[2-(4-chlorophenyl)-3-(pyridin-4-yl)-6,7-dihydropyrazolo[1,5-a]pyrazin-5(4H)-yl]-4-(4-methylpiperazin-1-yl) but-2-en-1-one
(E)-1-(2-(4-chlorophenyl)-3-(pyridin-4-yl)-6,7-dihydropyrazolo[1,5-a]pyrazin-5(4H)-yl)-4-morpholinobut-2-en-1-one
(2E)-1-[2-(4-chlorophenyl)-3-(pyridin-4-yl)-6,7-dihydropyrazolo[1,5-a]pyrazin-5(4H)-yl]-4-[isopropyl(methyl)amino]but-2-en-1-one
(2E)-4-(tert-butylamino)-1-[2-(4-chlorophenyl)-3-(pyridin-4-yl)-6,7-dihydropyrazolo[1,5-a]pyrazin-5(4H)-yl]but-2-en-1-one
(2E)-1-[2-(4-chlorophenyl)-3-(pyridin-4-yl)-6,7-dihydropyrazolo[1,5-a]pyrazin-5(4H)-yl]-4-(4-methoxypiperidin-1-yl) but-2-en-1-one
(2E)-1-[2-(4-chlorophenyl)-3-(pyridin-4-yl)-6,7-dihydropyrazolo[1,5-a]pyrazin-5(4H)-yl]-4-[(2-methoxyethyl)(methyl)amino]but-2-en-1-one
(2E)-1-[2-(4-chlorophenyl)-3-(pyridin-4-yl)-6,7-dihydropyrazolo[1,5-a]pyrazin-5(4H)-yl]-4-[(2-hydroxyethyl)(methyl)amino]but-2-en-1-one
(2E)-1-[2-(4-chlorophenyl)-3-(pyridin-4-yl)-6,7-dihydropyrazolo[1,5-a]pyrazin-5(4H)-yl]-4-(pyrrolidin-1-yl) but-2-en-1-one
(2E)-1-[2-(4-chlorophenyl)-3-(pyridin-4-yl)-6,7-dihydropyrazolo[1,5-a]pyrazin-5(4H)-yl]-4-(3-fluoroazetidin-1-yl) but-2-en-1-one
1-[2-(4-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-(2-(2-fluoro-4-(trifluoromethyl)phenyl)-3-(3-methyl-1H-pyrrolo[2,3-b]pyridin-4-yl)-6,7-dihydropyrazolo[1,5-a]pyrazin-5(4H)-yl) prop-2-en-1-one
1-[2-(4-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(3-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(3-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) prop-2-en-1-one
1-[2-(2-chlorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(5-chloro-2-fluorophenyl)-3-(3-methyl-1H-pyrrolo[2,3-b]pyridin-4-yl)-6,7-dihydropyrazolo[1,5-a]pyrazin-5(4H)-yl]prop-2-en-1-one
1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]prop-2-en-1-one
4-[3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
(2E)-1-[2-(4-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one
(2E)-1-[2-(3-chlorophenyl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one
(E)-4-(dimethylamino)-1-(3-(3-methyl-1H-pyrrolo[2,3-b]pyridin-4-yl)-2-(3-(trifluoromethyl)phenyl)-6,7-dihydropyrazolo[1,5-a]pyrazin-5(4H)-yl) but-2-en-1-one
(2E)-1-[2-(1-benzofuran-7-yl)-3-(3-methyl-1H-pyrrolo[2,3-b]pyridin-4-yl)-6,7-dihydropyrazolo[1,5-a]pyrazin-5(4H)-yl]-4-(dimethylamino)but-2-en-1-one formic acid
(2E)-4-(dimethylamino)-1-[2-(2-fluoro-1-benzothiophen-6-yl)-3-(3-methyl-1H-pyrrolo[2,3-b]pyridin-4-yl)-6,7-dihydropyrazolo[1,5-a]pyrazin-5(4H)-yl]but-2-en-1-one
1-[3-(pyridin-4-yl)-2-[6-(trifluoromethyl)pyridin-3-yl]-6,7-dihydropyrazolo[1,5-a]pyrazin-5(4H)-yl]prop-2-en-1-one
1-[2-phenyl-3-(pyridin-4-yl)-6,7-dihydropyrazolo[1,5-a]pyrazin-5(4H)-yl]prop-2-en-1-one
1-[2-(4-chloro-3-ethylphenyl)-3-(pyridin-4-yl)-6,7-dihydropyrazolo[1,5-a]pyrazin-5(4H)-yl]prop-2-en-1-one
1-{2-[4-chloro-2-(hydroxymethyl)phenyl]-3-(pyridin-4-yl)-6,7-dihydropyrazolo[1,5-a]pyrazin-5(4H)-yl}prop-2-en-1-one
1-[2-(1-benzothiophen-5-yl)-3-(pyridin-4-yl)-6,7-dihydropyrazolo[1,5-a]pyrazin-5(4H)-yl]prop-2-en-1-one
1-[2-(1H-indol-5-yl)-3-(pyridin-4-yl)-6,7-dihydropyrazolo[1,5-a]pyrazin-5(4H)-yl]prop-2-en-1-one
1-[2-(1-benzofuran-5-yl)-3-(pyridin-4-yl)-6,7-dihydropyrazolo[1,5-a]pyrazin-5(4H)-yl]prop-2-en-1-one
1-[2-(1H-indol-6-yl)-3-(pyridin-4-yl)-6,7-dihydropyrazolo[1,5-a]pyrazin-5(4H)-yl]prop-2-en-1-one
(2E)-1-[2-(1-benzothiophen-5-yl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(1H-indol-5-yl)-3-(pyridin-4-yl)-6,7-dihydropyrazolo[1,5-a]pyrazin-5(4H)-yl]but-2-en-1-one
(2E)-1-[2-(1H-benzotriazol-6-yl)-3-(pyridin-4-yl)-6,7-dihydropyrazolo[1,5-a]pyrazin-5(4H)-yl]-4-(dimethylamino)but-2-en-1-one
(2E)-1-[2-(1-benzofuran-5-yl)-3-(pyridin-4-yl)-6,7-dihydropyrazolo[1,5-a]pyrazin-5(4H)-yl]-4-(dimethylamino)but-2-en-1-one
(2E)-4-(dimethylamino)-1-[2-(1H-indol-6-yl)-3-(pyridin-4-yl)-6,7-dihydropyrazolo[1,5-a]pyrazin-5(4H)-yl]but-2-en-1-one
rac-(R,E)-4-(dimethylamino)-1-(2-(4-fluorophenyl)-7-(hydroxymethyl)-3-(pyridin-4-yl)-6,7-dihydropyrazolo[1,5-a]pyrazin-5(4H)-yl) but-2-en-1-one
(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazine-7-carboxamide
1-[(7RS)-2-(4-fluorophenyl)-7-(hydroxymethyl)-3-(pyridin-4-yl)-6,7-dihydropyrazolo[1,5-a]pyrazin-5(4H)-yl]prop-2-en-1-one
(7RS)-5-acryloyl-2-(4-fluorophenyl)-3-(pyridin-4-yl)-4,5,6,7-tetrahydropyrazolo[1,5-a]pyrazine-7-carboxamide
5-acryloyl-2-(4-fluorophenyl)-N-methyl-3-(pyridin-4-yl)-4,5,6,7-tetrahydropyrazolo[1,5-a]pyrazine-7-carboxamide
5-acryloyl-2-(4-fluorophenyl)-N,N-dimethyl-3-(pyridin-4-yl)-4,5,6,7-tetrahydropyrazolo[1,5-a]pyrazine-7-carboxamide
1-[2-(4-fluorophenyl)-7,7-dimethyl-3-(pyridin-4-yl)-6,7-dihydropyrazolo[1,5-a]pyrazin-5(4H)-yl]prop-2-en-1-one
1-[2-(4-chloro-3-methylphenyl)-7,7-dimethyl-3-(pyridin-4-yl)-6,7-dihydropyrazolo[1,5-a]pyrazin-5(4H)-yl]prop-2-en-1-one
1-[2-(4-chlorophenyl)-7,7-dimethyl-3-(pyridin-4-yl)-6,7-dihydropyrazolo[1,5-a]pyrazin-5(4H)-yl]prop-2-en-1-one
1-[2-(4-fluorophenyl)-6,6-dimethyl-3-(pyridin-4-yl)-6,7-dihydropyrazolo[1,5-a]pyrazin-5(4H)-yl]prop-2-en-1-one
1-[2-(4-chlorophenyl)-6,6-dimethyl-3-(pyridin-4-yl)-6,7-dihydropyrazolo[1,5-a]pyrazin-5(4H)-yl]prop-2-en-1-one
1-[2-(4-chloro-3-methylphenyl)-6,6-dimethyl-3-(pyridin-4-yl)-6,7-dihydropyrazolo[1,5-a]pyrazin-5(4H)-yl]prop-2-en-1-one
(2E)-4-(dimethylamino)-1-[2-(4-fluorophenyl)-7,7-dimethyl-3-(pyridin-4-yl)-6,7-dihydropyrazolo[1,5-a]pyrazin-5(4H)-yl]but-2-en-1-one
(2E)-1-[2-(4-chloro-3-methylphenyl)-7,7-dimethyl-3-(pyridin-4-yl)-6,7-dihydropyrazolo[1,5-a]pyrazin-5(4H)-yl]-4-(dimethylamino)but-2-en-1-one
1-[(2E)-4-(dimethylamino)but-2-enoyl]-3′-phenyl-2′-(pyridin-4-yl)-5′,6′-dihydrospiro[piperidine-4,7′-pyrrolo[3,2-c]pyridin]-4′ (1′H)-one
(2E)-4-(dimethylamino)-1-[2-(4-fluorophenyl)-6,6-dimethyl-3-(pyridin-4-yl)-6,7-dihydropyrazolo[1,5-a]pyrazin-5(4H)-yl]but-2-en-1-one
(2E)-1-[2-(4-chlorophenyl)-6,6-dimethyl-3-(pyridin-4-yl)-6,7-dihydropyrazolo[1,5-a]pyrazin-5(4H)-yl]-4-(dimethylamino)but-2-en-1-one
(2E)-1-[2-(4-chloro-3-methylphenyl)-6,6-dimethyl-3-(pyridin-4-yl)-6,7-dihydropyrazolo[1,5-a]pyrazin-5(4H)-yl]-4-(dimethylamino)but-2-en-1-one rac-(R,E)-4-(dimethylamino)-1-(2-(4-fluorophenyl)-7-(2-hydroxyethyl)-3-(pyridin-4-yl)-6,7-dihydropyrazolo[1,5-a]pyrazin-5(4H)-yl) but-2-en-1-one
2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]acetamide
2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N-methylacetamide
2-[(7RS)-5-[(2E)-4-(dimethylamino)but-2-enoyl]-2-(4-fluorophenyl)-3-(pyridin-4-yl)-4,5,6,7-tetrahydropyrazolo[1,5-a]pyrazin-7-yl]-N,N-dimethylacetamide
1-(2-(4-fluorophenyl)-7-(2-hydroxyethyl)-3-(pyridin-4-yl)-6,7-dihydropyrazolo[1,5-a]pyrazin-5(4H)-yl) prop-2-en-1-one
2-(5-acryloyl-2-(4-fluorophenyl)-3-(pyridin-4-yl)-4,5,6,7-tetrahydropyrazolo[1,5-a]pyrazin-7-yl) acetamide
2-(5-acryloyl-2-(4-fluorophenyl)-3-(pyridin-4-yl)-4,5,6,7-tetrahydropyrazolo[1,5-a]pyrazin-7-yl)-N-methylacetamide
2-(5-acryloyl-2-(4-fluorophenyl)-3-(pyridin-4-yl)-4,5,6,7-tetrahydropyrazolo[1,5-a]pyrazin-7-yl)-N,N-dimethylacetamide
1-(2-(4-chloro-2-fluorophenyl)-6-methyl-3-(pyridin-4-yl)-6,7-dihydropyrazolo[1,5-a]pyrazin-5(4H)-yl) prop-2-en-1-one
1-[(6RS)-2-(4-methoxyphenyl)-6-methyl-3-(pyridin-4-yl)-6,7-dihydropyrazolo[1,5-a]pyrazin-5(4H)-yl]prop-2-en-1-one
1-[(RS)-2-(2-fluoro-1-benzothiophen-6-yl)-6-methyl-3-(pyridin-4-yl)-6,7-dihydropyrazolo[1,5-a]pyrazin-5(4H)-yl]prop-2-en-1-one
1-[(6RS)-2-[2-fluoro-4-(trifluoromethyl)phenyl]-6-methyl-3-(pyridin-4-yl)-6,7-dihydropyrazolo[1,5-a]pyrazin-5(4H)-yl]prop-2-en-1-one
1-[(RS)-2-(2-fluoro-4-methoxyphenyl)-6-methyl-3-(pyridin-4-yl)-6,7-dihydropyrazolo[1,5-a]pyrazin-5(4H)-yl]prop-2-en-1-one
1-[(RS)-6-methyl-2-(naphthalen-2-yl)-3-(pyridin-4-yl)-6,7-dihydropyrazolo[1,5-a]pyrazin-5(4H)-yl]prop-2-en-1-one
1-[(RS)-2-(2-chloro-1-benzothiophen-6-yl)-6-methyl-3-(pyridin-4-yl)-6,7-dihydropyrazolo[1,5-a]pyrazin-5(4H)-yl]prop-2-en-1-one
1-[(6RS)-2-(1-benzothiophen-6-yl)-6-methyl-3-(pyridin-4-yl)-6,7-dihydropyrazolo[1,5-a]pyrazin-5(4H)-yl]prop-2-en-1-one
(2E)-1-[(RS)-2-(4-chloro-2-fluorophenyl)-6-methyl-3-(pyridin-4-yl)-6,7-dihydropyrazolo[1,5-a]pyrazin-5(4H)-yl]-4-(dimethylamino)but-2-en-1-one
(2E)-1-[(RS)-2-(1-benzothiophen-6-yl)-6-methyl-3-(pyridin-4-yl)-6,7-dihydropyrazolo[1,5-a]pyrazin-5(4H)-yl]-4-(dimethylamino)but-2-en-1-one
1-[(RS)-2-(4-chloro-2-fluorophenyl)-6-methyl-3-(pyridin-4-yl)-6,7-dihydropyrazolo[1,5-a]pyrazin-5(4H)-yl]-2-fluoroprop-2-en-1-one
1-[(RS)-2-(1-benzothiophen-6-yl)-6-methyl-3-(pyridin-4-yl)-6,7-dihydropyrazolo[1,5-a]pyrazin-5(4H)-yl]-2-fluoroprop-2-en-1-one
1-[(RS)-2-(4-fluorophenyl)-4-methyl-3-(pyridin-4-yl)-6,7-dihydropyrazolo[1,5-a]pyrazin-5(4H)-yl]prop-2-en-1-one
1-[(RS)-2-(4-chloro-2-fluorophenyl)-4-methyl-3-(pyridin-4-yl)-6,7-dihydropyrazolo[1,5-a]pyrazin-5(4H)-yl]prop-2-en-1-one
1-[(RS)-2-(4-methoxyphenyl)-4-methyl-3-(pyridin-4-yl)-6,7-dihydropyrazolo[1,5-a]pyrazin-5(4H)-yl]prop-2-en-1-one
1-[(RS)-2-(1-benzothiophen-5-yl)-4-methyl-3-(pyridin-4-yl)-6,7-dihydropyrazolo[1,5-a]pyrazin-5(4H)-yl]prop-2-en-1-one
1-[(RS)-2-(1H-indol-6-yl)-4-methyl-3-(pyridin-4-yl)-6,7-dihydropyrazolo[1,5-a]pyrazin-5(4H)-yl]prop-2-en-1-one
(2E)-1-[(RS)-2-(4-chloro-2-fluorophenyl)-4-methyl-3-(pyridin-4-yl)-6,7-dihydropyrazolo[1,5-a]pyrazin-5(4H)-yl]-4-(dimethylamino)but-2-en-1-one
(2E)-1-[(6S)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one
(2E)-1-[(6R)-2-(4-chlorophenyl)-6-methyl-3-(pyridin-4-yl)-6,7-dihydropyrazolo[1,5-a]pyrazin-5(4H)-yl]-4-(dimethylamino)but-2-en-1-one
1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one
1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one
1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one
1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one
4-(dimethylamino)-1-[4-methyl-3-(1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one
(2E)-4-(dimethylamino)-1-[(6S)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
4-(dimethylamino)-1-[2-(4-fluorophenyl)-4-methyl-3-(1H-pyrrolo[2,3-b]pyridin-4-yl)-6,7-dihydropyrazolo[1,5-a]pyrazin-5(4H)-yl]but-2-en-1-one
(E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one
(2E)-4-(dimethylamino)-1-[(6S)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one
1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one
1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) prop-2-en-1-one
1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]prop-2-en-1-one
1-(2-(4-fluorophenyl)-4-methyl-3-(1H-pyrrolo[2,3-b]pyridin-4-yl)-6,7-dihydropyrazolo[1,5-a]pyrazin-5(4H)-yl) prop-2-en-1-one
1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) prop-2-en-1-one
1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]prop-2-en-1-one
(E)-4-(dimethylamino)-1-(4-methyl-3-(1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one
(E)-4-(dimethylamino)-1-(4-methyl-3-(3-methyl-1H-pyrrolo[2,3-b]pyridin-4-yl)-2-(4-(trifluoromethyl)phenyl)-6,7-dihydropyrazolo[1,5-a]pyrazin-5(4H)-yl) but-2-en-1-one
(2E)-4-(dimethylamino)-1-[(6R)-6-methyl-3-(3-methyl-1H-pyrrolo[2,3-b]pyridin-4-yl)-2-[4-(trifluoromethyl)phenyl]-6,7-dihydropyrazolo[1,5-a]pyrazin-5(4H)-yl]but-2-en-1-one
(E)-4-(dimethylamino)-1-(2-(4-fluorophenyl)-4-methyl-3-(1H-pyrrolo[2,3-b]pyridin-4-yl)-6,7-dihydropyrazolo[1,5-a]pyrazin-5(4H)-yl) but-2-en-1-one
(E)-4-(dimethylamino)-1-(2-(4-fluorophenyl)-4-methyl-3-(3-methyl-1H-pyrrolo[2,3-b]pyridin-4-yl)-6,7-dihydropyrazolo[1,5-a]pyrazin-5(4H)-yl) but-2-en-1-one
(2E)-4-(dimethylamino)-1-[(6R)-2-(4-fluorophenyl)-6-methyl-3-(3-methyl-1H-pyrrolo[2,3-b]pyridin-4-yl)-6,7-dihydropyrazolo[1,5-a]pyrazin-5(4H)-yl]but-2-en-1-one
1-[(6S)-2-(4-chloro-2-fluorophenyl)-6-methyl-3-(pyridin-4-yl)-6,7-dihydropyrazolo[1,5-a]pyrazin-5(4H)-yl]prop-2-en-1-one
1-[(6R)-2-(4-chloro-2-fluorophenyl)-6-methyl-3-(pyridin-4-yl)-6,7-dihydropyrazolo[1,5-a]pyrazin-5(4H)-yl]prop-2-en-1-one
1-[2-anilino-3-(pyridin-4-yl)-6,7-dihydropyrazolo[1,5-a]pyrazin-5(4H)-yl]prop-2-en-1-one
(2E)-1-[2-anilino-3-(pyridin-4-yl)-6,7-dihydropyrazolo[1,5-a]pyrazin-5(4H)-yl]-4-(dimethylamino)but-2-en-1-one
1-[2-(4-methoxyphenyl)-7-methyl-3-(pyridin-4-yl)-6,7-dihydropyrazolo[1,5-a]pyrazin-5(4H)-yl]prop-2-en-1-one
1-[(6S)-2-(1-benzothiophen-5-yl)-6-methyl-3-(pyridin-4-yl)-6,7-dihydropyrazolo[1,5-a]pyrazin-5(4H)-yl]prop-2-en-1-one
1-[(6R)-2-(1-benzothiophen-5-yl)-6-methyl-3-(pyridin-4-yl)-6,7-dihydropyrazolo[1,5-a]pyrazin-5(4H)-yl]prop-2-en-1-one
1-[2-(4-chloro-2-fluorophenyl)-4-methyl-3-(pyridin-4-yl)-6,7-dihydropyrazolo[1,5-a]pyrazin-5(4H)-yl]prop-2-en-1-one
1-[(7RS)-2-(1-benzothiophen-5-yl)-7-methyl-3-(pyridin-4-yl)-6,7-dihydropyrazolo[1,5-a]pyrazin-5(4H)-yl]prop-2-en-1-one
1-[(7RS)-2-(4-chlorophenyl)-7-methyl-3-(pyridin-4-yl)-6,7-dihydropyrazolo[1,5-a]pyrazin-5(4H)-yl]prop-2-en-1-one
1-[2-(4-chloro-2-fluorophenyl)-7-methyl-3-(pyridin-4-yl)-6,7-dihydropyrazolo[1,5-a]pyrazin-5(4H)-yl]prop-2-en-1-one
3-[(6S)-6-methyl-3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
3-[(6R)-6-methyl-3-(3-methyl-1H-pyrrolo[2,3-b]pyridin-4-yl)-5-(prop-2-enoyl)-4,5,6,7-tetrahydropyrazolo[1,5-a]pyrazin-2-yl]benzonitrile
(2E)-1-[(2-(4-chloro-2-fluorophenyl)-4-methyl-3-(pyridin-4-yl)-6,7-dihydropyrazolo[1,5-a]pyrazin-5(4H)-yl]-4-(dimethylamino)but-2-en-1-one
(2E)-1-[2-(4-chloro-2-fluorophenyl)-7-methyl-3-(pyridin-4-yl)-6,7-dihydropyrazolo[1,5-a]pyrazin-5(4H)-yl]-4-(dimethylamino)but-2-en-1-one
3-[(6S)-5-[(2E)-4-(dimethylamino)but-2-enoyl]-6-methyl-3-(3-methyl-1H-pyrrolo[2,3-b]pyridin-4-yl)-4,5,6,7-tetrahydropyrazolo[1,5-a]pyrazin-2-yl]benzonitrile
3-[(6R)-5-[(2E)-4-(dimethylamino)but-2-enoyl]-6-methyl-3-(3-methyl-1H-pyrrolo[2,3-b]pyridin-4-yl)-4,5,6,7-tetrahydropyrazolo[1,5-a]pyrazin-2-yl]benzonitrile
1-[(6R)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one
1-[(6S)-2-(4-chloro-2-fluorophenyl)-4,6-dimethyl-3-(pyridin-4-yl)-6,7-dihydropyrazolo[1,5-a]pyrazin-5(4H)-yl]prop-2-en-1-one
or an N-oxide, a salt, a tautomer, a rotamer, or a stereoisomer of said compound, or a salt of said N-oxide, tautomer, rotamer, or stereoisomer.

4. A method for the treatment or prophylaxis of disease in a subject comprising administering the compound of formula (I) according to claim 1.

5. The method according to claim 4, wherein the diseases is a hyperproliferative disease and/or disorder responsive to induction of cell death.

6. The method according to claim 5, wherein the hyperproliferative disease and/or disorder responsive to induction of cell death are haematological tumours, solid tumours and/or metastases thereof.

7. The method according to claim 6, wherein the tumour harbors a mutant EGFR and/or metastases thereof.

8. The method according to claim 6, wherein the tumour is lung cancer.

9. The method according to claim 5, wherein the tumour is lung cancer harboring a mutant EGFR with in-frame deletion in exon 19, exon 20 insertion mutation, or point mutation in exon 21 and/or
a mutant ERBB2 with exon 20 insertion mutation, and/or metastases thereof.

10.-13. (canceled)

14. A pharmaceutical composition comprising at least one compound of formula (I) according to claim 1, together with at least one pharmaceutically acceptable auxiliary.

15. (canceled)

16. A combination comprising one or more first active ingredients selected from a compound of formula (I) according to claim 1, and one or more second active ingredients selected from chemotherapeutic anti-cancer agents and target-specific anti-cancer agents.

17.-18. (canceled)

19. A method of inhibiting EGF receptor kinase activity and/or reducing the survival of a cancer cell or inducing death in a cancer cell, the method comprising contacting a cancer cell comprising a mutation in an EGF-receptor with a compound of formula (I) according to claim 1.

20.-22. (canceled)

23. A method of treating cancer in a subject, the method comprising administering to the subject an effective amount of a compound of formula (I) according to claim 1.

24. The method according to claim 23, wherein the cancer is or has acquired resistance to an anti-EGF receptor therapy.

25. A method of enhancing the efficacy of an anti-EGF-receptor therapy for the treatment of cancer, the method comprising administering to the subject an anti-EGF receptor therapy in combination with a compound of formula (I) according to claim 1.

26. The method of claim 23, wherein the cancer is selected from the group consisting of leukemia, myelodysplastic syndrome, malignant lymphoma, head and neck tumours, tumours of the thorax, gastrointestinal tumours, endocrine tumours, mammary and other gynaecological tumours, urological tumours, skin tumours, and sarcomas.

27. The method of claim 26, wherein the cancer is selected from the group consisting of inverted sinonasal papilloma or inverted sinonasal papilloma associated sinanonasal squamous cell carcinoma.

28. The method of claim 26, wherein the tumour of the thorax is non-small cell lung cancer.

29. The method of claim 23, wherein the cancer comprises a EGF-receptor mutation.

30.-35. (canceled)

36. A method for treating a patient with cancer, the method comprising administering to the subject an anti-EGF receptor therapy in combination with a compound of formula (I) according to claim 1, wherein the subject is selected for therapy by detecting the presence of a mutation in exon 20 of the EGF-receptor in a biological sample of the subject.

37.-43. (canceled)
